SEQUENCE LISTING (1440 sequences)

SEQ ID NO: 1 - DNA, 48 bp, Artificial Sequence
  Affymetrix ID 3326487
ggaaagacgagaactatttatatgacaccaactatggtagcacagtag

SEQ ID NO: 2 - DNA, 34 bp, Artificial Sequence
  Affymetrix ID 2458376
tttttgacaacaggtcctatgattcattacacag

SEQ ID NO: 3 - DNA, 65 bp, Artificial Sequence
  Affymetrix ID 3414753
agatgattttgtaaaagtactattttaaatatcaaatcaaccaataaattcacatttgtgttagg

SEQ ID NO: 4 - DNA, 37 bp, Artificial Sequence
  Affymetrix ID 2332285
agtgcattgtacagcatattttcatgaataaaattgt

SEQ ID NO: 5 - DNA, 303 bp, Artificial Sequence
  Affymetrix ID 2478155
cctgtgatgtgtagtaaccattatattgtttgtatgaggtagtaactaaattattttggccatgtattaatactctaagtcaaaagaaatatgaaaaggatcataaaataaggccaacaaaagtaaaaattccaagagaaatttgaaccacttcactctatggaatgttacagttcttcagtgtgatcatatgaaatgtttagtgaggactctttaataatgctaattaattctttgtgcatactgtaattctgaccacaattgcagtattctatcatggtaccgctattctgtgattcaaaa

SEQ ID NO: 6 - DNA, 30 bp, Artificial Sequence
  Affymetrix ID 3139092
gtgagttccggtattactgaaatgaaacag

SEQ ID NO: 7 - DNA, 69 bp, Artificial Sequence
  Affymetrix ID 2789391
cagattggtactcaggtgatacataaacagcatccagatcctgattcttcagtgaagatacaaaaccct

SEQ ID NO: 8 - DNA, 141 bp, Artificial Sequence
  Affymetrix ID 3463598
cttaccagcgttataggccagtatcaacttcaagttcaaccactccatcctcttcactttctactatgagcagttcactgtatgcttcaagtcaactaaacaggccaaatagtcttgtaggcataacttctgcttactcca

SEQ ID NO: 9 - DNA, 93 bp, Artificial Sequence
  Affymetrix ID 2461837
ctttgaacaggcacttgaatttcacaaaagtagaactattcctgctaactggaagactgaattgaaagaagatagctctagcagtgaagcaga

SEQ ID NO: 10 - DNA, 46 bp, Artificial Sequence
  Affymetrix ID 2512364
attaaccttgcaagaactcttcaggcacatatggaagatctcgaaa

SEQ ID NO: 11 - DNA, 25 bp, Artificial Sequence
  Affymetrix ID 2364325
accagagctgacaatgacactctgg

SEQ ID NO: 12 - DNA, 139 bp, Artificial Sequence
  Affymetrix ID 3066770
tacaagcttattcacattttgcttcctaatctttttgttgtacagggattcaggtttcttattcttacaacatgattgtttatatgtgaagcacatcttgctgttgccttatttttgatgcttttattcatgacaagaa

SEQ ID NO: 13 - DNA, 41 bp, Artificial Sequence
  Affymetrix ID 2956442
ccatttgtcagtttagcattacattgtcttgagcaccagaa

SEQ ID NO: 14 - DNA, 34 bp, Artificial Sequence
  Affymetrix ID 3005357
gttaatattgtcatcgatacaaataaagtgaaat

SEQ ID NO: 15 - DNA, 81 bp, Artificial Sequence
  Affymetrix ID 2651515
atgatgatgctgttaactacattcaacaaaaatcctttaaaacagctgttttcaaccaactttcgctgtgaatgtactttt

SEQ ID NO: 16 - DNA, 64 bp, Artificial Sequence
  Affymetrix ID 2318654
tggctcctgcattaacccagaaatacctcgcttctatctgtgcacttagctgggaacttaccca

SEQ ID NO: 17 - DNA, 96 bp, Artificial Sequence
  Affymetrix ID 2318755
tcagaagtcgctgtccttactacttttgcggaagtatggaagtcacaactacacagagatttctcagcctacaaattgtgtctatacatttctaag

SEQ ID NO: 18 - DNA, 69 bp, Artificial Sequence
  Affymetrix ID 2320062
attgcgcagtctctttgtggagaggacttgatcattaaaggaatcagcgttcatatatccaatgccgaa

SEQ ID NO: 19 - DNA, 38 bp, Artificial Sequence
  Affymetrix ID 2321661
ttttgaaatgccatgcaatggagctttttgtaataaaa

SEQ ID NO: 20 - DNA, 45 bp, Artificial Sequence
  Affymetrix ID 2322383
tttcctgtgtgcaatcagtaccttgaaggcagaacattctgaata

SEQ ID NO: 21 - DNA, 173 bp, Artificial Sequence
  Affymetrix ID 2325809
caacagccctgcatgggtttgtttgtttttttactgctcactcccaaccttttgtaatgccattttctaaacttatttctgagtgtagtctcagcttaaagttgtgtaatactaaaatcacgagaacacctaaacaacaaccaaaaatctattgtggtatgcacttgattaac

SEQ ID NO: 22 - DNA, 43 bp, Artificial Sequence
  Affymetrix ID 2327494
tgtaatttacctaacaaattgcctaatctgctatgttaatgta

SEQ ID NO: 23 - DNA, 34 bp, Artificial Sequence
  Affymetrix ID 2327865
gggaggcataagctgatgtttgtaaagcgctttg

SEQ ID NO: 24 - DNA, 124 bp, Artificial Sequence
  Affymetrix ID 2328504
tcctaattggatttgtaccgtcctcccattttgttctcggaagattaaatgctacatgtgtaagtctgcctaaataggtagcttaaacttatgtcaaaatgtctgcagcagtttgtcaataaag

SEQ ID NO: 25 - DNA, 130 bp, Artificial Sequence
  Affymetrix ID 2328507
gtgacagcaacttgcataaacaattctttagctgttaattttaatgttaaaactttgcaaaacttgtttaataaaaatagctgtaaaaagaaaaaccatatgttgccgcataaattagccataactctta

SEQ ID NO: 26 - DNA, 44 bp, Artificial Sequence
  Affymetrix ID 2329019
gatttaacactggttttgagacagactttattcagctatccctc

SEQ ID NO: 27 - DNA, 31 bp, Artificial Sequence
  Affymetrix ID 2329798
tccaacagtaacagcgaaaatcatcggtgat

SEQ ID NO: 28 - DNA, 34 bp, Artificial Sequence
  Affymetrix ID 2330704
tacctcctgtaacacatcaataaagtacaatcat

SEQ ID NO: 29 - DNA, 101 bp, Artificial Sequence
  Affymetrix ID 2331419
cttggccagggattaattcagagtgcaggaaaagactgtgatgtacagggtttagaacatgacatggaagagatcaatgctcgatggaatacattgaataa

SEQ ID NO: 30 - DNA, 81 bp, Artificial Sequence
  Affymetrix ID 2331842
tcctaaggctaaagaggagcagtcctatgcttttattcagcatcctttatctgtgacttcatgctctgataactgcctttc

SEQ ID NO: 31 - DNA, 29 bp, Artificial Sequence
  Affymetrix ID 2332192
ccagactcgatgtgtactctaacttaaga

SEQ ID NO: 32 - DNA, 52 bp, Artificial Sequence
  Affymetrix ID 2332740
tggaagtgtcagagattcagaatccaagattgtctttaagttttcaactgta

SEQ ID NO: 33 - DNA, 53 bp, Artificial Sequence
  Affymetrix ID 2334302
tgtatacctttaccctcaagtaccactaacacagatgattgatcgtttccagg

SEQ ID NO: 34 - DNA, 359 bp, Artificial Sequence
  Affymetrix ID 2336960
tgtagtgtcattaaaggtcatttcctgctaaatttcaaattacagatttgtgggccattcctgagcagaagcatcatttctactaaatattcagcttgttaactaaggtaactgacagtatcatggcaggaggtgagaaggagcaatgatcagctcatagctaagaaaagggaaaaaaacaataaccaaagcaaaaaacaaaatagctacagatattagtaaaataataatacaatcccaaacatccacctcttaaattactacaaaaacaataccaataaagttatagcaaatacagtcttcacagatttgagtaactttatttgcattttatagtgatttcttaaggcctatatcca

SEQ ID NO: 35 - DNA, 170 bp, Artificial Sequence
  Affymetrix ID 2339310
caaagtgcattaactcttgttggaatattttatggctattccaaagtatagagtgcctaaattttgtgttgaaaatgtctttcatgacggtattaagttttctgaaattacacatggcttttaaaatttctaatgtatccaagatgtgcaatgttgttaggatctcattc

SEQ ID NO: 36 - DNA, 60 bp, Artificial Sequence
  Affymetrix ID 2342617
tatgactgttggtctctaggtagcctttggtctattgtacacaatctcatttcatatgtt

SEQ ID NO: 37 - DNA, 194 bp, Artificial Sequence
  Affymetrix ID 2345634
ttggatgatatcaaggatcgaattaagagagaaataaggaaagaactgaaaatcaaagaaggagctgaaaatctgaggaaagtcacaacagataaaaaaagtttggcttatgtagacaacattttgaaaaaatcaaataaaaaattagaagaactacatcacaagctgcaggaattaaatgcacatattgttgt

SEQ ID NO: 38 - DNA, 79 bp, Artificial Sequence
  Affymetrix ID 2345663
ttttttgctgcagcatacccaaagaatacaacacactctagtcaatacagtgattcccaaatagatacaggtcagaatc

SEQ ID NO: 39 - DNA, 39 bp, Artificial Sequence
  Affymetrix ID 2345666
tttaatccagttattgaaagaagaccaaaacttcaaaga

SEQ ID NO: 40 - DNA, 34 bp, Artificial Sequence
  Affymetrix ID 2346911
tcagaagaaggatcgggtagctcaaaagaaggca

SEQ ID NO: 41 - DNA, 43 bp, Artificial Sequence
  Affymetrix ID 2346912
tttctatgattttttcagatatagataataaacttatgaacag

SEQ ID NO: 42 - DNA, 161 bp, Artificial Sequence
  Affymetrix ID 2347108
ctagggatgtctgctattaagtttcatctattgtgtgctatacatgtaaaaactgtctctttgaactattgaaaatttaaggttcagtataatatcaattttgaatttttaatggtgtttatgaaattttagatagcagcgagtccttcgtttgatcaata

SEQ ID NO: 43 - DNA, 72 bp, Artificial Sequence
  Affymetrix ID 2348819
ttttattgtgtcaagcaatactaatttgtgtcataaaatttgttttgcatcattcaacaatttatgcccaga

SEQ ID NO: 44 - DNA, 150 bp, Artificial Sequence
  Affymetrix ID 2350305
acgaagtcgaagtggcagtcaaggaagaactgacagtgttgaaaaatcaaaaaaacgggaacatagtcccagcaaagaaaaatctagaaagcgtagtagaagcaaagaacgttcccacaaacgagatcacagtgatagtaaggaccagtc

SEQ ID NO: 45 - DNA, 49 bp, Artificial Sequence
  Affymetrix ID 2350371
gatgacctctgggttagaattcgacatcgacatattgcggttgtgttag

SEQ ID NO: 46 - DNA, 1210 bp, Artificial Sequence
  Affymetrix ID 2352268
tgttgaacctatagcgttgtcttgattcttttgtgttctctgccttgtaattttctgttactgctatatctacgtgtaaatctttttttctttttttttttttttttttggttaattctgccacatttaatgttggtgagagagtgatctatcctaatgacattttactgtttaaaaaagtttcctagccatgaagccctgctactgatttagacaaggtattatggtcattactttgtacccctatccttccaagcacttctggtacttcagtcgtttttactgatccaccaacacctaaagaggctatgctacagtctctagctaaatggaagacacattcatccttctccctctgactgctttgatcatcatttattgcatctcataactaattttctaaagtttggattgggacttttcaggtcctttttggagggcaaaggaagtgccagcttctctggggaacttgtttttaaatccaaagacttgaaccacattccctgcacatgaacatgtttgcttttatcccttctctcattgtctccttcccatcttagtaccattgtagttataaacatctgcattttttagaagcattttacccatttatttttttaaacattcaagaactgctgacgtactgtggatgtagagtataaaacttgaaaaatgcagatgttgaaggaataataggtatcttgtgctttaatactttatggcaggattgtactataagcaaatgaattaaacagctatgtaaatcataaagaaaaactaaaaatgaaccaaagtgaaaggataacttccaggcagtatctttctattgtaacctgttatttaaggaaatactagtgatttcttctaaataggatgtaaaacttctttcaaattactcttcctcagtcctgcctgccaagaactcaagtgtaactgtgataaaataacctttcccaggtatattggcaggtatgtgtgtaatctcagaatacacaggtgacatagatatgatatgacaactggtaatggtggattcatttacattgtttacacttctatgaccaggccttaagggaaggtcagttttttaaaaaaccaagtagtgtcttcctacctatctccagatacatgtcaaaaaaagaaaggtgtttgtgcttccgttttgtttctgctcagtaatatagtcaagcaagtttgttccaggtgaccc

SEQ ID NO: 47 - DNA, 28 bp, Artificial Sequence
  Affymetrix ID 2353492
gttggacgtgataagtatgagcctgcag

SEQ ID NO: 48 - DNA, 45 bp, Artificial Sequence
  Affymetrix ID 2358275
ggatctggcaaagatcagttccatccttagcagtttaacatcagt

SEQ ID NO: 49 - DNA, 71 bp, Artificial Sequence
  Affymetrix ID 2358923
attatccagaactgaagttgccctacttttaactttgaacttggctagttcaaagatagactcttcttttg

SEQ ID NO: 50 - DNA, 82 bp, Artificial Sequence
  Affymetrix ID 2359857
catggagatggacaaccatatgtcggataaggatgagagttgctatgacaatgcagaggcagccttcagtgacgatgaagag

SEQ ID NO: 51 - DNA, 554 bp, Artificial Sequence
  Affymetrix ID 2360078
ataaagtcagaccagtgcattgggcttgttaggggaaaccattccttcctactgtcacatagcctggcagattatatcaggtgccaaaacattcccttttattcaatttattaatgagagtgtggaattaaagtagatccttgcatgtcaggaaagctgcaacacatctggtttaagtggagcacaaaggtaagttattccacattcaccaacttccccacttgacctcccaccagttcctgtctgctttttccatcaatcaactctaagatgccctaaacccaaacagttcccattcaactgtgcacaaaggtatagatatggaagacatctcactccaactctcacctttggaattaaataaacctggaacagggaaggtgaactatggctgaaaccaagtgaagatttttaagaagctttgacagaaacattaagaataacacctgaatgcaaatccagaaaaaagcgctactgggggaaaatagtcttagccaatgttctaaaaatgctcataaggaagggttggggaattaccctttagacacaagctc

SEQ ID NO: 52 - DNA, 118 bp, Artificial Sequence
  Affymetrix ID 2360119
aggctctgcagtgaaatctgattcaccttccacttctagcatcccccctctcaatgaaacggtatctgcagcttccttactgacgacaaccaatcagcattcatcctccttgggtggc

SEQ ID NO: 53 - DNA, 28 bp, Artificial Sequence
  Affymetrix ID 2360120
gtgaggagattccaaatactaccaccac

SEQ ID NO: 54 - DNA, 236 bp, Artificial Sequence
  Affymetrix ID 2360340
ccatagcataggtaaggaggctctagtctggagcacagctgagtttccagcaatataaggaggctcgaaagtttcttttataagaatgcctgctagcaagggttccagcaaggtggttggttggtctgtaagtcagtcttgagtacttgaaacagttctgtgtttgttttttttccttagcgtttagaatagccatcattgtcctgcaataggcagagctatcacgtccaggaaaa

SEQ ID NO: 55 - DNA, 50 bp, Artificial Sequence
  Affymetrix ID 2360716
atcgtgccattgtgatatgaatatgccttatatgctgatatgaatatgcc

SEQ ID NO: 56 - DNA, 348 bp, Artificial Sequence
  Affymetrix ID 2360962
tggcaggtcgacatacatagttctccaagttctttggaggttttttagttctcttagcctggaggccaattttcaattttaaatttccctctgaaaagtttgtttctttcactgaaaactgttgctgtgcatcagtcaaaccatcatctttcccagcttcgatgtttctttctcgattccgtttgcgaaggtcctcttcctcctttgtgtttttttctagctctacttctctcttactgaccaatgtgccagtactgatggcagaaggactctttcttgaaaaaccttcggaatcagaacccaatcctaacatagcagtatttctagggtccatcacaagcgtatgtt

SEQ ID NO: 57 - DNA, 34 bp, Artificial Sequence
  Affymetrix ID 2361031
tattcactgatccttagtaacatgttaacattta

SEQ ID NO: 58 - DNA, 32 bp, Artificial Sequence
  Affymetrix ID 2363064
taaaattaggcctctaacagtgatagggacag

SEQ ID NO: 59 - DNA, 221 bp, Artificial Sequence
  Affymetrix ID 2363334
aggtccattgtaagctagagaagtaaattccaaggctggcaataactgactcatattcttcacaagtggcctagacaataaggaaccattcacctcaaattcacagagccatgaatcacctctgcttccccatgaccttttccatatccttcctactctgtcttccaaccatgacacagaactgaaacatactttaaaaatctcatccttggctaggcacg

SEQ ID NO: 60 - DNA, 33 bp, Artificial Sequence
  Affymetrix ID 2363956
gttaaatcatgaacttcgaggatgggttcatag

SEQ ID NO: 61 - DNA, 30 bp, Artificial Sequence
  Affymetrix ID 2365991
gtggtgtatgcggatatccgaaagaattaa

SEQ ID NO: 62 - DNA, 71 bp, Artificial Sequence
  Affymetrix ID 2367178
agatggaagtagattatacagagcaactgaatttcagtgatgatgatgaacaaggaagtaacagtcctaaa

SEQ ID NO: 63 - DNA, 34 bp, Artificial Sequence
  Affymetrix ID 2367214
tcccagccttctatatggacacaagtcatttatt

SEQ ID NO: 64 - DNA, 52 bp, Artificial Sequence
  Affymetrix ID 2367236
tgggatgagtatcttgttcatagctgaaaatgcagaagttctggctcattcc

SEQ ID NO: 65 - DNA, 313 bp, Artificial Sequence
  Affymetrix ID 2369830
ggctttgtcatttaccctttgagagttccacaagtggtagtagagtggtttaacgtctttcctctagtactaccagtattcataaatgtataccccttactgtaatttgttcctcttagaagtcagatcatctgatttatagaggattatgaaaaccagagtttttgaaaaggcttattttatacatacgtattatatagagaaacaaatgtttttattaaatgtttcattgacccaagtaatttaaaagtaatatgttacctatgtctttcaggaaaaataattatagcagctgatctgtaaatgactttaa

SEQ ID NO: 66 - DNA, 52 bp, Artificial Sequence
  Affymetrix ID 2370014
cattggtgtgttctttaacttgctacttggagacctagtgtccagtctggac

SEQ ID NO: 67 - DNA, 117 bp, Artificial Sequence
  Affymetrix ID 2371291
cagggctaaaatcagtgctatctacaagccgaaatttaagcaacaactgtgacacaggagagaagccagtggttaccttcaaagaaaacattaagacacgagaagtgaacagagacc

SEQ ID NO: 68 - DNA, 60 bp, Artificial Sequence
  Affymetrix ID 2371339
gttttagagctactcgatatttataactttttataagcaccggtcattttttgagaactg

SEQ ID NO: 69 - DNA, 145 bp, Artificial Sequence
  Affymetrix ID 2371796
aaaggtgcacttcgtcagggtatgaagacaggtggttgaaatgaaggcaggtgtgtcttaagctctaatggtgattccaaaactatcaggcttgaaaatgatgtaatcaagtccttaaaagctatagacacgaatttcttctgtg

SEQ ID NO: 70 - DNA, 34 bp, Artificial Sequence
  Affymetrix ID 2373002
atgcttaatgcaaaagaccttctacaggacctga

SEQ ID NO: 71 - DNA, 40 bp, Artificial Sequence
  Affymetrix ID 2375035
gaaatttgggcctttggatcgaatatggtcaagaggttgg

SEQ ID NO: 72 - DNA, 37 bp, Artificial Sequence
  Affymetrix ID 2375037
tggaaataccaatcagattgttggctgaagtgatgtg

SEQ ID NO: 73 - DNA, 74 bp, Artificial Sequence
  Affymetrix ID 2375675
ggttactaattgtatgatagtgtttttatatggaagaatgtacagcttatggacaaatgtacacctttttgtta

SEQ ID NO: 74 - DNA, 32 bp, Artificial Sequence
  Affymetrix ID 2375780
ccaatactgctgtgcactgagaataaaaactc

SEQ ID NO: 75 - DNA, 154 bp, Artificial Sequence
  Affymetrix ID 2375850
gagccagtcagaaacgtggagaattgcaaactaaactcaagacagaaggaccttcaaaaactgatgattctacttcaggagcaagaagctcctccactatccgtatcaaaaccttctctgaggtcctggctgaaaaaaaacatcggcagcagga

SEQ ID NO: 76 - DNA, 26 bp, Artificial Sequence
  Affymetrix ID 2375890
tcgtgttactacaagatggcaataaa

SEQ ID NO: 77 - DNA, 236 bp, Artificial Sequence
  Affymetrix ID 2377473
tgccagatgaatgacacgaaattcacataaaattctgctgttgtgattttttgtgcttttccagggttcttagcacgttatgtacattgcatgggtatatgcttttaatatttttatgtataaaaagtgaattacaacaactttttggaattgaaacatgggcatttttatctaagtaagtcaacaatggcataattcatataaatgaaatgagagcaataactcccaagtggttg

SEQ ID NO: 78 - DNA, 131 bp, Artificial Sequence
  Affymetrix ID 2377508
agagaccagttttctctggaagtttgtttaaatgacagaagcgtatatgaattcaagaaaatttaagctgcaaaaatgtatttgctataaaatgagaagtctcactgatagaggttctttattgctcattt

SEQ ID NO: 79 - DNA, 31 bp, Artificial Sequence
  Affymetrix ID 2378615
gttcagaatgctaagcaagtaaacagtgcac

SEQ ID NO: 80 - DNA, 30 bp, Artificial Sequence
  Affymetrix ID 2379787
tttctcatttgggatcagcgatatttcctg

SEQ ID NO: 81 - DNA, 248 bp, Artificial Sequence
  Affymetrix ID 2383763
aggcagtttctggtactcctatgcaatattactcagctttttttattgtaaaaagaaaaatcaactcactgttcagtgctgagaggggatgtaggcccatgggcacctggcctccaggagtcgctgtgttgggagagccggccacgcccttggctttagagctgtgttgaaatccattttggtggttggtttttaacccaaactcagtgcattttttaaaatagttaagaatccaagtcgagaacact

SEQ ID NO: 82 - DNA, 331 bp, Artificial Sequence
  Affymetrix ID 2389076
gccagacctaagagtatgatatgtatgtactatatggttccatttatacatgaatttctacagtgagctatttaaggtggtagaaatcattttatgtagacatagcattattatcccgttaaaaacgagagcaaatacttgtttcttataatagccaatgctatcagaagacttaaaaactaatggattttatcactttacaaattgttatgtagacatcctttttttcctaataaaaaaagaatgtgttgataggtattctgacaccaaaactgcccaaaaatagcactataaattagaacatatttttcagtctcagggtagtagtcat

SEQ ID NO: 83 - DNA, 174 bp, Artificial Sequence
  Affymetrix ID 2389079
gtctactgccaggatagcattcttacgtgttacatatagtggacttgtcatccttaaaatgtgaacagaatttattggcagtgtggcaaagaattataaaacatagtgtttaatgtacttggagtttccttgtagtagtaagtatagagtttgatgataagtaaacgtccctta

SEQ ID NO: 84 - DNA, 70 bp, Artificial Sequence
  Affymetrix ID 2389084
caagaaaatgtgactatccctaggtaccatcatttgcaagcatctctttagtctacgtacctctactcca

SEQ ID NO: 85 - DNA, 130 bp, Artificial Sequence
  Affymetrix ID 2389086
tgagagaggtttctattgttcactttagaaaaatgtaggtcaatgtaatgggtgatatctgttcttaattattctaagaattaaatcctgtatgttatcagtcacctattatatggcacaccctcagggg

SEQ ID NO: 86 - DNA, 105 bp, Artificial Sequence
  Affymetrix ID 2389817
ctgaagtcataacgtgcgtgaattaacagcttctctatttgatatttgaaattcttctgtaagcctgtctgagtgtatgtggaaacgattgtcaaatctaaaata

SEQ ID NO: 87 - DNA, 53 bp, Artificial Sequence
  Affymetrix ID 2394560
aactcttgtaatcgagttttgatcatattttctattaaagtggctaacacctg

SEQ ID NO: 88 - DNA, 861 bp, Artificial Sequence
  Affymetrix ID 2395192
tcgtctcccatgctagcaagtaggaagttcggacagtttcataacatggcccattcacaattccccattgaaatttagaggcaggtcaccttctatgaatacacaaagacactattgtggtcagaagtgagctggcttagtgaacacaattctttttatactaaaaaaatttttttcttaagaaagctaacaagtaggtgatggaaacaatgaataaaaaataactttttctagaacatataaataattttaagtgaccattgaaatgtaagtttagaattccaaggcaatttctgcagaggcgatagttacacaatcattctgttgaaagctaagatttagatggcattaggaggctgacacacagtaattactagtagtacttgttggcttgcagacaggtgggggttgggcctgcagaatcccctgcagcattttcctgtaatctggatgagcgatcttcaaacgtgtgctttcaaactacttaaataccatattcttcctatcttgcctttcatttttatattaccacagatactttcctcatagtcttgccagtgcttgtagaatgcttagaaaaagcttgataaaccactgggctaagtacacagagggagaggctagcagtatttttaaattggtttctaaattttttatagcttgatggtagataacacatttgcttcattgaagtaatctgaaaaaccaatcctcaaaagacctctcaattagaattcttaaatgacaatgttttctttatcatatatttgagagattgatttaaagaaaaataatgcttgactatctgaaataatattttaaccctatcataaaatctctgcctggtagaacagctgactg

SEQ ID NO: 89 - DNA, 664 bp, Artificial Sequence
  Affymetrix ID 2395195
gcatccgtggatcgttaaagactgagggcttcaaaatagttgtttgaagtggtaatagaatgtcaaattgatgccttaacccacccattttagaggatacataggccagatctacaattaataacttttaataggagaatttgagggagttgaagtgaaacaggaactttcataggctacaggttacccaagaaaaactattaccagtaaatccataaaaaaggatctgtgctacccttacaagttgagcagtatagtcagtcggatcctacttcgccagtttaaaaagccgtagggacaggaagaggcagtgtggtgtgttggtgaagggcctggacactgaggccgggctgtctagttgaagtcctggccccattgctcaccagttttgaccttgagcaagttctttaactctgtgcctcaagtaagtgagagtaataatagtagcccaccttatggcattgtagagaggattaaatgagttcatacacataaagcacttagaaccatgcttgcctggcagttgtatgtccagtgaatgttagttatcattaatggttttatattaattgtaacaatctagagttacccatcgggtaacttaaatgcttgttaaataaatttgaagttttaaagacttgcgtcccatcattagaccattgtg

SEQ ID NO: 90 - DNA, 36 bp, Artificial Sequence
  Affymetrix ID 2396421
aactatattccctggatcgagaacataattagtgat

SEQ ID NO: 91 - DNA, 41 bp, Artificial Sequence
  Affymetrix ID 2400180
ggagagaataagaacggcggtaacagttattggcaaaaagc

SEQ ID NO: 92 - DNA, 196 bp, Artificial Sequence
  Affymetrix ID 2400323
cccttgagtactgccagaggcaaaagatgtttgtttcttattcattccacttttgtctcctgggatcccttctgtagcctaaagtatggctgggaaatggacttgagaagattggcttgaattagatcataatcatgtgtgatcccatcatgaattcattggaatttgtgttgcatgtaaggcaatctttcctgtt

SEQ ID NO: 93 - DNA, 311 bp, Artificial Sequence
  Affymetrix ID 2401277
tgaaaaggactagttctgcaaaatctatgagaacaagagcttctgcaaatgcataatataaattctcttccttttttttttaaactgtatttttacaaacctagatctcttcctgctttgatttgtctttatcatatctttcagaacaaacctttgatggaattctaggaaaggttgagattaagaatatattaaaaatattccactttgtacattctgtttatattatagtaaactaagttatttagcaagataaaattaggaatttcacccaaggtatcaatcagatggaaccattagtccactgaaga

SEQ ID NO: 94 - DNA, 218 bp, Artificial Sequence
  Affymetrix ID 2401288
caagatacgattggctctagatctacattcttcaaaaaaaaaaattggcttaactgtttcatctttaagtagcattttgctgccatttgtattgggctgaagaaatcactattgtgtatatactcaagtctttttatttttcctcttttcataaatgctcttggacattattgggcttgcagagttcccttattctggggattacaatgcttttatcg

SEQ ID NO: 95 - DNA, 41 bp, Artificial Sequence
  Affymetrix ID 2401350
aaagaggaagcgctgaattatctgaactggagaagcaataa

SEQ ID NO: 96 - DNA, 29 bp, Artificial Sequence
  Affymetrix ID 2401368
tcctctccaaaaagaccatcggtggaaag

SEQ ID NO: 97 - DNA, 368 bp, Artificial Sequence
  Affymetrix ID 2402210
ttgtggttttggacctcagatatgacaaatacttacattgacaaatccataatttcttttgtaatttctttttatttttacaaattataccatgataaaatttgacaaaaattattcatgtgaaagtttcctctaacattttataagttaatcaagtgcataccacaatagatttttggttgttgtttaggtgttctcgtgattttagtattacacaactttaagctgagactacactcagaaataagtttagaaaatggcattacaaaaggttgggagtgagcagtaaaaaaacaaacaaacccatgcagggctgttgtgctgtgggaaatcagatgtgttcactgccataagtcttcagtgcggcc

SEQ ID NO: 98 - DNA, 34 bp, Artificial Sequence
  Affymetrix ID 2405379
aagagaagcttcctcaggctagcaaaaatatcat

SEQ ID NO: 99 - DNA, 44 bp, Artificial Sequence
  Affymetrix ID 2406094
tctgatgagacgacaggaagaattaagacgcatggaagaacttc

SEQ ID NO: 100 - DNA, 38 bp, Artificial Sequence
  Affymetrix ID 2406148
gtccattgtgggatgattaaacatggtatttaactgtg

SEQ ID NO: 101 - DNA, 78 bp, Artificial Sequence
  Affymetrix ID 2407134
ttattcatatcatgccttatggaaatttctttttctgtattttctctctttgctggtattcacctgattaaatattgc

SEQ ID NO: 102 - DNA, 128 bp, Artificial Sequence
  Affymetrix ID 2408880
gcaaccacagtacaaccttccagaaagagacaagcaactacttttctcagaatataattttgaagatcttagtgcctcatttcggagcctttataagtcagtttttgagcagtcacttagtcaacaag

SEQ ID NO: 103 - DNA, 37 bp, Artificial Sequence
  Affymetrix ID 2410112
cccattccctgagaatcctgttgttagtaaagtgctt

SEQ ID NO: 104 - DNA, 37 bp, Artificial Sequence
  Affymetrix ID 2410528
atttccaaagctggataagttggtcattgattaaaaa

SEQ ID NO: 105 - DNA, 41 bp, Artificial Sequence
  Affymetrix ID 2412669
gtcaagtcatcaactgtatttttgtactggcattaacaaaa

SEQ ID NO: 106 - DNA, 73 bp, Artificial Sequence
  Affymetrix ID 2413580
tctaaaattccactcctcatagagcttttaaaatggtttcattggatataggccttaagaaatcactataaaa

SEQ ID NO: 107 - DNA, 31 bp, Artificial Sequence
  Affymetrix ID 2414030
aattcattgctgattgctacacaagattaga

SEQ ID NO: 108 - DNA, 305 bp, Artificial Sequence
  Affymetrix ID 2414960
gacaacccgggatcgtttgcaagtaactgaatccattgcgacattgtgaaggcttaaatgagtttagatgggaaatagcgttgttatcgccttgggtttaaattatttgatgagttccacttgtatcatggcctacccgaggagaagaggagtttgttaactgggcctatgtagtagcctcatttaccatcgtttgtattactgaccacatatgcttgtcactgggaaagaagcctgtttcagctgcctgaacgcagtttggatgtctttgaggacagacattgcccggaaactcagtctattta

SEQ ID NO: 109 - DNA, 372 bp, Artificial Sequence
  Affymetrix ID 2417788
caccgtgtgcataagattactactgcagtaacacaaatttattggttttatacaacttgagatcaaataaaggtacattatcaagtagatttgtcttattttaaaagcgtatgaaggtcaatttaaaatgcactaccccttttttccaacacggagcttaaaacaaattaatgaaagagtggaaaattcaaaataagggcaagagataaggtttttttctttttttcctttaagatagactcaggataggtagatagctttcactgatgtagatgtggaataaattattacttcaggaaaaaaattcccaaacatcttatgaaaaagtatacaactctacttcaaaatatgctatttactcactgccaaaga

SEQ ID NO: 110 - DNA, 72 bp, Artificial Sequence
  Affymetrix ID 2418028
tgcagcaatgtgaattgggccagaagatcagagtgtaatatgtgtaatactccaaagtatgctaaattagaa

SEQ ID NO: 111 - DNA, 389 bp, Artificial Sequence
  Affymetrix ID 2419242
gcaaatgccatacgttgtgtgaatattatgttggaatacagtgctgatatcttggaaaaccataactgcctcttaatttaacatagaataatacatagttctgtattttttttaaagtgagcttaatgggtaagtattttttatatgctttagctatagctaaagaaaactgatacttaacaaagttgaatagtattattcactggtgctcctaaaatattgtttttcagtgtaaaatatgcatatcttctatatttaatatgaaagtcttgaaatgtatcagacagaaggggatttcagtttgcaaataatgagcaatgtagcaattttaacacatttcataaatatatattttgtcattggtggagagcaccatttgttgttttgaa

SEQ ID NO: 112 - DNA, 154 bp, Artificial Sequence
  Affymetrix ID 2419247
cagtatagtggtagattggaggatcttttccatttattgtattggctctttcagcacaagtaatcctgatatcttcattttttttccttctgtttgattaaaaactgcatgtgtgtacaatgatcttttggcatacttccattgcattaacagt

SEQ ID NO: 113 - DNA, 100 bp, Artificial Sequence
  Affymetrix ID 2419276
cccattccaagatttgctgttggcattgtaataggaagaaatggagagatgatcaaaaaaatacaaaatgatgctggtgttcgcattcagtttaagccag

SEQ ID NO: 114 - DNA, 30 bp, Artificial Sequence
  Affymetrix ID 2420740
ttgtaatgtcttaaagtttcaaatctgttt

SEQ ID NO: 115 - DNA, 69 bp, Artificial Sequence
  Affymetrix ID 2420839
agagaagttctgcactgatttttagtctcttgatgtctcaatcttacatgtataccaatcacaatggaa

SEQ ID NO: 116 - DNA, 556 bp, Artificial Sequence
  Affymetrix ID 2421273
gatcagtcttttacttgaggcattaaatatctaattaaatcgtgaaatggcagtatagtccatgatatctaaggagttggcaagcttaacaaaacccattttttataaatgtccatcctcctgcatttgttgataccactaacaaaatgctttgtaacagacttgcggttaattatgcaaatgatagtttgtgataattggtccagttttacgaacaacagatttctaaattagagaggttaacaagacagatgattactatgcctcatgtgctgtgtgctctttgaaaggaatgacagcagactacaaagcaaataagatatactgagcctcaacagattgcctgctcctcagagtctctcctatttttgtattacccagctttctttttaatacaaatgttatttatagtttacaatgaatgcactgcataaaaactttgtagcttcattattgtaaaacatattcaagatcctacagtaagagtgaaacattcacaaagatttgcgttaatgaagactacacagaaaacctttctagggatttgtgtggatca

SEQ ID NO: 117 - DNA, 42 bp, Artificial Sequence
  Affymetrix ID 2421274
agaactgagcattctcaaatggaacacagacagtgtagaaga

SEQ ID NO: 118 - DNA, 94 bp, Artificial Sequence
  Affymetrix ID 2421932
agcaaaatttgcctgtccagctccctctccccaagaaacaacatgaatgagcaacttcagagtgtcaaacaactgccattaaacttaactcaaa

SEQ ID NO: 119 - DNA, 64 bp, Artificial Sequence
  Affymetrix ID 2422519
tgtgattatatgtacgaaaatcgtgtaacactatgctcatgctaagaaccgacataacagaatt

SEQ ID NO: 120 - DNA, 40 bp, Artificial Sequence
  Affymetrix ID 2423200
tctggcccatgatgtgcttccgatgtacttctgcattaaa

SEQ ID NO: 121 - DNA, 39 bp, Artificial Sequence
  Affymetrix ID 2427009
tactggtaattttctgtatatttggaatatttgggttaa

SEQ ID NO: 122 - DNA, 84 bp, Artificial Sequence
  Affymetrix ID 2427501
ttaatgtgcattaggcacttttctgtttatttaagagtcaattgctttctaatgctctatggaccgactatcaagatattagta

SEQ ID NO: 123 - DNA, 41 bp, Artificial Sequence
  Affymetrix ID 2427978
ctgcttattcagaagtgatcacggaacaactcaaaggcaga

SEQ ID NO: 124 - DNA, 585 bp, Artificial Sequence
  Affymetrix ID 2428394
tgggtcacctggaacaaacttgcttgactatattactgagcagaaacaaaacggaagcacaaacacctttctttttttgacatgtatctggagataggtaggaagacactacttggttttttaaaaaactgaccttcccttaaggcctggtcatagaagtgtaaacaatgtaaatgaatccaccattaccagttgtcatatcatatctatgtcacctgtgtattctgagattacacacatacctgccaatatacctgggaaaggttattttatcacagttacacttgagttcttggcaggcaggactgaggaagagtaatttgaaagaagttttacatcctatttagaagaaatcactagtatttccttaaataacaggttacaatagaaagatactgcctggaagttatcctttcactttggttcatttttagtttttctttatgatttacatagctgtttaattcatttgcttatagtacaatcctgccataaagtattaaagcacaagatacctattattccttcaacatctgcatttttcaagttttatactctacatccacagtacgtcagcagttcttg

SEQ ID NO: 125 - DNA, 470 bp, Artificial Sequence
  Affymetrix ID 2429071
cccggtggggttagattactcatctgtccaccaagtgggacatttgcatggactgggggcttaaaggacttagaagagacctgtaagtaaatcctgaaaatgagccaatccccacttgaatggttactggagtaaacccacctttaccaccccaattacagcacccgaggccgataaaccaacttggctctggttcatttttcttttcttcatttgtgatgctcagattcaaaatgtgtgttctacactgttacaggcttctcttttgtttgattaaagattttagtcctacttttgtatggacacattagaatattcagagaccaaaatagaagaatttgctgttagatatttttcagaagtcagcagatttgtggcaaatcatttatttgcctttttaaaaattcatttaagcagttcagagagtagactactcagaaaattatttcacgtaattgtctaagaggtca

SEQ ID NO: 126 - DNA, 362 bp, Artificial Sequence
  Affymetrix ID 2429119
cattggaaactcactttagctgtggttttgtgtggctgttattacatcttaagaaagttgagcgtctactctgtatattgttttttaatgtgtgctgtttctagtcccccgaccctccatcttttaaatttatattgactgtttcatatagaaatcttagcaaatgattcattgtctggaataatttcatagatttttagttgtttaattaagccaaattgtgcattagcaacagacttttaggatgctaatgtgtccagtagttaggtcctgaggaatgtttgaaagataagagtagctcttttcacatggaagagaaatggattttccttttgttacagcgtagtatggacctaaagacc

SEQ ID NO: 127 - DNA, 30 bp, Artificial Sequence
  Affymetrix ID 2429168
tgagattgaacggactattgttcagctaga

SEQ ID NO: 128 - DNA, 123 bp, Artificial Sequence
  Affymetrix ID 2429282
ggtggcgccttatagtatcaattttaggagctttatttggtgcatttaacgcaactggtaattgcagaatccactttgcctgtgtaagtgaaaaatatagactgttatcttgttggccctatg

SEQ ID NO: 129 - DNA, 113 bp, Artificial Sequence
  Affymetrix ID 2429309
ctttggagacaaagatacgaaatccaaggtgaccctgctggaaggtgaccatgttaggtttaatatttcaacagaccgacgtgacaaattagagcgagcaaccaatatagaag

SEQ ID NO: 130 - DNA, 90 bp, Artificial Sequence
  Affymetrix ID 2429310
gcaacagatgtcagactattgcctcaaggaacagtcatttttgaagatatcagcattgaacattttgaaggaactgtaaccaaagttatc

SEQ ID NO: 131 - DNA, 32 bp, Artificial Sequence
  Affymetrix ID 2433376
ctgatttataaaagcaggatgtgttgatttta

SEQ ID NO: 132 - DNA, 75 bp, Artificial Sequence
  Affymetrix ID 2434721
taaagccagaattacggctagcacctagcatttcagcagagggaccattttagaccaaaatgtactgttaatggg

SEQ ID NO: 133 - DNA, 123 bp, Artificial Sequence
  Affymetrix ID 2435391
gcagaaatgagcagttcgctcctccctgataagagttgtcccaaagggtcgcttaaggaatctgccccacagcttcccccatagaaggatttcatgagcagatcaggacacttagcaaatgta

SEQ ID NO: 134 - DNA, 28 bp, Artificial Sequence
  Affymetrix ID 2437095
ccctgaatgttcaataaagttgattctt

SEQ ID NO: 135 - DNA, 28 bp, Artificial Sequence
  Affymetrix ID 2437537
tcccaccattttcgagaactcatgcagc

SEQ ID NO: 136 - DNA, 36 bp, Artificial Sequence
  Affymetrix ID 2437538
gttaacatgttactaaggatcagtgaatacagccct

SEQ ID NO: 137 - DNA, 50 bp, Artificial Sequence
  Affymetrix ID 2437680
tttaggactgaagcattttgaaggaactgagtttcctaatcctctaatca

SEQ ID NO: 138 - DNA, 56 bp, Artificial Sequence
  Affymetrix ID 2437775
tcatgctgaatactctagatttgtgaatcagattaatactgctgtacctttaccag

SEQ ID NO: 139 - DNA, 95 bp, Artificial Sequence
  Affymetrix ID 2439041
cttggatcttttacgacggatgggagatctgctaaaccttcttctcaagggtgttcttgatcgccttaatctgattggtgagatactgaagcttc

SEQ ID NO: 140 - DNA, 30 bp, Artificial Sequence
  Affymetrix ID 2440149
caaagatgtgattggtttaaggatcagtcc

SEQ ID NO: 141 - DNA, 33 bp, Artificial Sequence
  Affymetrix ID 2440513
tagtgttgtgcacacaaaagcatcaaataaaga

SEQ ID NO: 142 - DNA, 472 bp, Artificial Sequence
  Affymetrix ID 2442702
gatgaaactcagatggagcttcctactcacgttgatttatgtctcacaatcctgggtattgttaattcaacatagggtgaaactatttctgataaagaacttttgaaaaactttttatactctaaagtgatactcagaacaaaagaaagtcataaaactcctgaatttaatttccccacctaagtcgaaacagtattatcaaaacacatgtgcacacagattattttttggctccaaaactggattgcaaaagaaagaggagaagaatattttgtgtgttcctggtattcttttataagtaaagtttacccaggcatggaccagcttcagccagggacaaaatcccctcccaaaccactctccacagctttttaaaaatacttctactcttaacaattacctaaggcttcctcaactgccccaaatctcttaatagcttctagtgctgctacaatctaagtcaggtcaccag

SEQ ID NO: 143 - DNA, 265 bp, Artificial Sequence
  Affymetrix ID 2443917
attccttgattagctgtatactgtattaatctagctcattaagcaccctctattcagtaaaacttctgagaatatataaattctacaaataacaaacaattgtctattaacaaagcttctttgcataagtagctaatattgtatcaacaaattcatactgttattagtaaaagatatcacaacttacctgttattctctttaggactgttacttccttgttcatcatcatcactgaaattcagttgctctgtataatctacttcc

SEQ ID NO: 144 - DNA, 30 bp, Artificial Sequence
  Affymetrix ID 2444488
gactcagaattcatgattgaagaaatgcag

SEQ ID NO: 145 - DNA, 125 bp, Artificial Sequence
  Affymetrix ID 2444489
tctggcggtaacattctagtttatgccccgaaaaggggaatatagccattctataatgtttggagattttggattactcctaattgtatgcaagttgtcttactgtgtattgtcccttaatttca

SEQ ID NO: 146 - DNA, 130 bp, Artificial Sequence
  Affymetrix ID 2448289
atcttgtgagtcagctaagacagacagaagagcaggtgaatgacttaaaggagagactcaaaacaagtacgagcaatgtggaacaatatcaagcaatggttactagtttagaagaatccctgaacaagga

SEQ ID NO: 147 - DNA, 36 bp, Artificial Sequence
  Affymetrix ID 2448313
cttgaaattgctcaggatcgcaatattgccattcag

SEQ ID NO: 148 - DNA, 30 bp, Artificial Sequence
  Affymetrix ID 2450669
aaggaagccatcattaaattgttttatttc

SEQ ID NO: 149 - DNA, 274 bp, Artificial Sequence
  Affymetrix ID 2451314
ggactccaaccaagaggatttcttcaaatctcagcaaagctacaggactggtactcaagccagcctgtaaacggtgctatttctattccttatgggatcatttttccaggactctttgaagaaaagaaaaaacaactaaaaaaatttttgacactttttgtattttttccttaagagctatttgtggttgttgaggtttgaaaagctgactgttttttttgcaggggttcccaccaatttggaaggcattgaagcttgcaccttttcatgtaca

SEQ ID NO: 150 - DNA, 28 bp, Artificial Sequence
  Affymetrix ID 2452652
gatgtgaagaccaagaaggatgattctc

SEQ ID NO: 151 - DNA, 40 bp, Artificial Sequence
  Affymetrix ID 2452655
aggaaggttgttgattactcacagtttcaggaatctgatg

SEQ ID NO: 152 - DNA, 49 bp, Artificial Sequence
  Affymetrix ID 2457626
tgactgagaatgtaagtctgcgctctgattaacacaatggagaaacgga

SEQ ID NO: 153 - DNA, 98 bp, Artificial Sequence
  Affymetrix ID 2458075
ctgctatggtcacggttctaaggatgatttaagaagtctctggctcctcaactgtgcactgtagtgtaggctgacttcaaccacggcttctccagctt

SEQ ID NO: 154 - DNA, 40 bp, Artificial Sequence
  Affymetrix ID 2458381
gaagaattgctgaaaagggatcaacaatagaaacagaaca

SEQ ID NO: 155 - DNA, 25 bp, Artificial Sequence
  Affymetrix ID 2458503
agcttctgaacagcccacttagtta

SEQ ID NO: 156 - DNA, 42 bp, Artificial Sequence
  Affymetrix ID 2459655
attttcaagtacaacattctgctcaacatcatttacacttga

SEQ ID NO: 157 - DNA, 116 bp, Artificial Sequence
  Affymetrix ID 2461827
ttagaatctatacctacacattctgatcaggaaaaagaagttaacattaaaaaaccagaagacaatgaaaatctggatgacaaagatgatgacacaactagggtagatgaatccct

SEQ ID NO: 158 - DNA, 168 bp, Artificial Sequence
  Affymetrix ID 2461828
gagtactgtagatcagccaacattgaatttcagatggcattgccagagaaagttgttaacaagcaatgtaaggagtgtgaaaatgtaaaagaaataaaagttaaggaggaaaatgaaacagagatcaaagaaataaagatggaggaggagaggaatataataccaaga

SEQ ID NO: 159 - DNA, 106 bp, Artificial Sequence
  Affymetrix ID 2462178
aattcttaaactgtggaaaccactgaaggtgcttattaactgttctcccagatttgtacaagtattggatgattccttgagtttacagctgtacaaatagtgtgga

SEQ ID NO: 160 - DNA, 399 bp, Artificial Sequence
  Affymetrix ID 2464496
acctgggttgatgactactggactattttaagatacctacttccaaaacttattttattctagaggaccactaaaaatcagaattacagcaatcattttaatttttctactttccttgttaaggtcttctgcttacccaaagaatagtgtatatcttaacatgacttttaaaggggtatcttgatccccatcacagatttttatctatgaatagaaggattgaaaccctatttgatccctagctctttgctgaaaagtgcataaacaggaatcctgaatttattatatagacctcctcgcattatataatcgaagcaaagggaaaccagcgatggagtagaggtacgtagactaaagagatgcttgcaaatgatggtacctagggatagtcacattttc

SEQ ID NO: 161 - DNA, 30 bp, Artificial Sequence
  Affymetrix ID 2464505
tggctgggagaagttaacactgagtttatc

SEQ ID NO: 162 - DNA, 38 bp, Artificial Sequence
  Affymetrix ID 2464514
agccatggagtcagcattatcaccaaggatattattga

SEQ ID NO: 163 - DNA, 86 bp, Artificial Sequence
  Affymetrix ID 2464537
cagagaagatcccagtaaggcatttatatacaaaagatattgacatacatgaagttcgtattggctggtcactaactacaagtgga

SEQ ID NO: 164 - DNA, 113 bp, Artificial Sequence
  Affymetrix ID 2468935
gaatcacccagaactacatgacattccaatatactatgcatcatctttggccaagaagtgtatggcagtgtaccagacatatgtaaatgccatgaatgacaaaatccgcaaac

SEQ ID NO: 165 - DNA, 167 bp, Artificial Sequence
  Affymetrix ID 2469568
gctgctgtatttcatgtcttaacggctattttgaggttcattaacaacatagaaagccttgaactgtataaccagctagattccttaataattagtcactagagacagcccaaagacaaatattgggcaggaaatcagttctcactgagcccggtttccatgtaaaa

SEQ ID NO: 166 - DNA, 95 bp, Artificial Sequence
  Affymetrix ID 2473241
ttgatacaccaaaatcggcaagctatcttaaaccagtttgcagcaactgctcctgttggcatcaatatgagatcaggcatgcaacagcaaattac

SEQ ID NO: 167 - DNA, 63 bp, Artificial Sequence
  Affymetrix ID 2473624
ttttgtacaacagtggaattttctgtcatggataatgtgcttgagtccctataatctatagac

SEQ ID NO: 168 - DNA, 35 bp, Artificial Sequence
  Affymetrix ID 2474147
gtatgccctaccaggtggaataaaggacacagatt

SEQ ID NO: 169 - DNA, 38 bp, Artificial Sequence
  Affymetrix ID 2474700
attttatgcaagaatcgatggcacaatactggaagaga

SEQ ID NO: 170 - DNA, 45 bp, Artificial Sequence
  Affymetrix ID 2474770
gttcttcatattaggacagatatcattgcatcacatttatttatc

SEQ ID NO: 171 - DNA, 217 bp, Artificial Sequence
  Affymetrix ID 2475647
taagttcctcctatttgccactgggctgttggttagaagcataggtaactgattaagtaggtatgatactgcatttgaaataagtggacacaaactatcctttctccaccatggactcaatctgagaacaacagcattcatttccattcatttccatactggcttttgattatatgcagattcctagtagcatgccttacctacagcactatgtgca

SEQ ID NO: 172 - DNA, 90 bp, Artificial Sequence
  Affymetrix ID 2476316
tacaacccttacattggaggtctgggaattcctgtagcaaagccaccagcaaacacggagaagaacggatcacagacagttagcgtttca

SEQ ID NO: 173 - DNA, 31 bp, Artificial Sequence
  Affymetrix ID 2476408
ctggagacttgcacttgtatggatgaattta

SEQ ID NO: 174 - DNA, 38 bp, Artificial Sequence
  Affymetrix ID 2477341
tttattacaaatccactgagaagtctggaatgtatgga

SEQ ID NO: 175 - DNA, 192 bp, Artificial Sequence
  Affymetrix ID 2478839
acgcaggaagcgttccagctatttaatgctggcaactactgtttaatggtcagttaaatctgtgataatggttggaagtgggtggggttatgaaattgtagatgtttttagaaaaacttgtgaatgaaaatgaatccaagtgtttcatgtgaagatgttgagccattgctatcatgcattcctgtctcatgg

SEQ ID NO: 176 - DNA, 615 bp, Artificial Sequence
  Affymetrix ID 2479729
tcatgactacgcatctaacagcttcaaaatatagaaaatactcaaatgcaaaatcaacaaatctgcaattacagaaaaagacttcaacatacttttagaaagtagtaactcaagtagacaataatagctaacacttaccgggcacttcttatgtgagtggcactgaaacatgtgctttcatgcattacctaaggctagagaaaatgttaataacttagacaagtcatacatgtatcataaatatctttgtacctgacaaacagaataaacatctttttaaacaccattagataattatttaaagaccacatattgggaacctacccctaaagaacaataacaaccacaataacaactatcaccccagtaaacatggctggcaagaatacaattaaatgaacattatcactgatttggcaacagctcttatgaagaataatttggcattatgtatcaagtgcccatactctttgacaaagtaactgtctttctgataatctatcttaagtaatacaaaaatggggggaggggaataaaaatacaaaatattagaaacactatcgtcaaaatgaaagggcatggctgagtatggtacaaccgttcaacagaatattg

SEQ ID NO: 177 - DNA, 77 bp, Artificial Sequence
  Affymetrix ID 2480989
actgatttgtgattgaaagctgcctttctatttacttgagtcttgtacatacatacttttttatgagctatgaaata

SEQ ID NO: 178 - DNA, 74 bp, Artificial Sequence
  Affymetrix ID 2481178
ctgactacattggaagctttgagttgacttctgacaaaggtggtaaattcagacaacattatgatctaataaac

SEQ ID NO: 179 - DNA, 128 bp, Artificial Sequence
  Affymetrix ID 2482593
ggatgtatcatctgttgaactcttaatgaataatcatcaaggcatcaaagctgaaattgatgcacgtaatgacagtttcacaacctgcattgaacttgggaaatccctgttggcgagaaaacactatg

SEQ ID NO: 180 - DNA, 75 bp, Artificial Sequence
  Affymetrix ID 2482624
ctggtgctcagtcgtacgacctgtacctctcaacttttgccctatctgttaaatatatgctatgtcattaaatgc

SEQ ID NO: 181 - DNA, 28 bp, Artificial Sequence
  Affymetrix ID 2483047
taacaaaatggaagttcaaattgtctta

SEQ ID NO: 182 - DNA, 59 bp, Artificial Sequence
  Affymetrix ID 2484558
tgagaatattgcctgggaagattacattagacgatgtatatgcgaagacacttgatagc

SEQ ID NO: 183 - DNA, 31 bp, Artificial Sequence
  Affymetrix ID 2484807
ttgtgcccgcattggtattaaatcctcgcat

SEQ ID NO: 184 - DNA, 139 bp, Artificial Sequence
  Affymetrix ID 2485214
tctttgactcaagtaccaaataataataatgctatacttaaattacttgtgaaaaacaacacattttaaagattacgtgcttcttggtacaggtttgtgaatgacagtttatcgtcatgctgttagtgtgcattctaaa

SEQ ID NO: 185 - DNA, 84 bp, Artificial Sequence
  Affymetrix ID 2485276
ttaatgaactcttttaacacggatgaagataccaaaaaaatactacagaagtacaatcattgtcgtgtgaaaatctacactttc

SEQ ID NO: 186 - DNA, 32 bp, Artificial Sequence
  Affymetrix ID 2487216
tggaatagggaactgcaaatgtatgatgattt

SEQ ID NO: 187 - DNA, 241 bp, Artificial Sequence
  Affymetrix ID 2487645
gctgttctcccatgatccaactgtgtaatttctggtcagtgattccaggttttaaataatttgtaagtgttcagtttctacacaactttatcatccgctaagaatttaaaaatcacattctctgttcagctgttaatgctgggatccatatttagttttataagcttttccctgtttttagttttgttttgggttttttggctcatgaattttatttctgtttgtcgataagaaatgtaag

SEQ ID NO: 188 - DNA, 93 bp, Artificial Sequence
  Affymetrix ID 2488150
cgttctagaagatcaagctcaagtcacagattccgtcggtctcgaagcccaatgcattacatgtataggccgagaagtcgaagtccaagaatt

SEQ ID NO: 189 - DNA, 46 bp, Artificial Sequence
  Affymetrix ID 2489030
caaggatcccctcgagactactctgttaccagtcatgaaacattaa

SEQ ID NO: 190 - DNA, 40 bp, Artificial Sequence
  Affymetrix ID 2489435
ctgactcctgggctctgaagaatcacagaaacacttttta

SEQ ID NO: 191 - DNA, 30 bp, Artificial Sequence
  Affymetrix ID 2491645
atgtccttgtgatgtgcacgtaaagtactt

SEQ ID NO: 192 - DNA, 32 bp, Artificial Sequence
  Affymetrix ID 2492101
cactctatgatgttggagacattgttcattct

SEQ ID NO: 193 - DNA, 39 bp, Artificial Sequence
  Affymetrix ID 2492833
ctgctcacatgttgggaatccctggaataaaatgcttgt

SEQ ID NO: 194 - DNA, 41 bp, Artificial Sequence
  Affymetrix ID 2495140
ttcaggcaaccgtgttaaattattgtacatatctgagagag

SEQ ID NO: 195 - DNA, 50 bp, Artificial Sequence
  Affymetrix ID 2498988
ttgccaagaaacagatgatgctaatacagaaagctaaatcaaggtgtaca

SEQ ID NO: 196 - DNA, 39 bp, Artificial Sequence
  Affymetrix ID 2507241
ttaaaatttcacgttgatccagtcttaactttaaaatac

SEQ ID NO: 197 - DNA, 72 bp, Artificial Sequence
  Affymetrix ID 2507514
cacttcctctcaggagcctagtggatgctcagatcagagacctgcagaggacctcaacatccgagtggaaag

SEQ ID NO: 198 - DNA, 239 bp, Artificial Sequence
  Affymetrix ID 2507533
gaaataactctctgctaggtccttgcttatatggcaaccactgctagaaccctaaaagaaccaaaaatctgccacagcctgcctccatcagcttcttatttagtatttcatatgcccattagccctatgcttcagatgacacgttttgtttagagctactttgctccaagactcttaagcccaaagtaactggtatgtcactgagtaacttgactcggtgtcagagcattttaactagc

SEQ ID NO: 199 - DNA, 30 bp, Artificial Sequence
  Affymetrix ID 2512351
tgttcaagacagagttccttcatattcaca

SEQ ID NO: 200 - DNA, 50 bp, Artificial Sequence
  Affymetrix ID 2512363
atccatttgcaacatttaaactactgtatttctttctgcattgtttctag

SEQ ID NO: 201 - DNA, 143 bp, Artificial Sequence
  Affymetrix ID 2512959
gaagacatagttcacctaaaatggcatcctgctctgaatctagactttttagaaatggcatatgtttttgatgatatgtcaacattcaaaattgtcctaattaaattgttgtttaaatgtaatgtcaactctttataaactta

SEQ ID NO: 202 - DNA, 87 bp, Artificial Sequence
  Affymetrix ID 2515085
gtgtaccaacagtaccgttattgccaccacaagtaaaccagtccctcacttctgtgccaccaatgaatccagctactacattaccag

SEQ ID NO: 203 - DNA, 25 bp, Artificial Sequence
  Affymetrix ID 2517330
tcctaaataacttcaagtgtggtta

SEQ ID NO: 204 - DNA, 138 bp, Artificial Sequence
  Affymetrix ID 2518193
taagcgcagtgcaccaaaatgtaactaagacttagtagcacctaggactcagatcttggtttttcttacaacattcgcactgaaaggaaccctggctctttggaaaaatgtttgattccaaggatgggattccttatg

SEQ ID NO: 205 - DNA, 845 bp, Artificial Sequence
  Affymetrix ID 2518707
gttatggcaggtacacttgaatttcaaaaatttaaaacatatataaaaagtggttagggactaacatttgtatcaacagttgataaatgtctaagattgtttattatacagcagggtacaatggtagtgctaatgccaacagggcaccatggaagttagtctaaaaattatcgctaggctttatacaagcaacaacatatgctgctgctcttagaattttgggaaatgatctgcttctaatactaagcagttctttaacatttttaaatgttatacagtgttacagtatttagtttggcaaatgtttcaaaataaagtagaaatgtattcataacatcacagaaatgcacatccaatgttgttttcaataagaaccataggtacagatcagaactcttccctatatgaaataatttacacacagatgaatactataatatcactatctaaaataatcactaaatacagttctttttcagaaataaacaagcaaagatttttttcattatcaaatatcaaaaacatgttgataatgtatgtttttcaaataaatgatactttaaaattataggaataatctcagtgaattcagtgaatgtgcaacctaaatcaaccaagtatactgtagtacaaccatattaagaaaccaatgatcagaaaatacaaccgtatgaaagaaatttataatccacaaaactttttcaggtcttaaggtaaaatagttccacagtctacaggtaaaaccaaggcaacaaaaatgcgtgcttatcttgattaagtaggtaattttatgcagaagatgtaacactttgtttgtagacagcatggtatgcatttctcagcaa

SEQ ID NO: 206 - DNA, 60 bp, Artificial Sequence
  Affymetrix ID 2525095
gcatattttagttagtactaaattcttagtaaaatgctgatcagtaaaccaatcccttga

SEQ ID NO: 207 - DNA, 45 bp, Artificial Sequence
  Affymetrix ID 2527206
gtaaagtccgccatcagaagactgaaggagttgaaagaccagtag

SEQ ID NO: 208 - DNA, 128 bp, Artificial Sequence
  Affymetrix ID 2527635
gtgctaataagtcaccactggtttataaataccccatctcattgtgaataccacatgtacctgtgaattagagccttggagacacttaacagacacaagaaaatgccaacagttacgtatctgttctg

SEQ ID NO: 209 - DNA, 29 bp, Artificial Sequence
  Affymetrix ID 2532844
cattatgttggccaacagaacttgattgt

SEQ ID NO: 210 - DNA, 63 bp, Artificial Sequence
  Affymetrix ID 2534447
gtccaagtacagtagacactcaaaatgaacccttagatatgaaagagcccgatgaagaaaaga

SEQ ID NO: 211 - DNA, 366 bp, Artificial Sequence
  Affymetrix ID 2537112
gctaagcatttgatctggccatgtatatggtagctgtgttttaatttgagaatcttgagggtagagccacaaatttcaattcttacatttccatttgcaaagtgactagagaaaaagaaatcagcttaaatgaggtattaagtaatgtttagagtcgtaggtattaactagaatataaatccttagaaattgtctttataccttcaaaaattatactatgcatttatcatagaaatgtgattacaaagaagtctgactaccatgtctttaaacatatggcatctctcaacttttcttccttatggggctacatttgttcatttccagcagtagcataaacttacggtgacatggtagacttgtctc

SEQ ID NO: 212 - DNA, 137 bp, Artificial Sequence
  Affymetrix ID 2539775
atcctgcaatcaagtagaagtcaacttcatctgaaagttcagctgttttcaaactgcaatgctgaaatgttatgcaaataatgaagttatcccttgctctagattttctgaagaaaatggattgtgtaaaatgctga

SEQ ID NO: 213 - DNA, 32 bp, Artificial Sequence
  Affymetrix ID 2540221
tgcctaccgccattcatgattaaaccatccag

SEQ ID NO: 214 - DNA, 117 bp, Artificial Sequence
  Affymetrix ID 2544180
cacgaatgacaactaccacctttttttcctttttaattaatactaaatattgtgatttcttatttgaggttcaaaatgacctgcttgaaactttgatacatattggaatacattatg

SEQ ID NO: 215 - DNA, 35 bp, Artificial Sequence
  Affymetrix ID 2544185
tttgggaaatatggacctattcgtcaaatcagagt

SEQ ID NO: 216 - DNA, 70 bp, Artificial Sequence
  Affymetrix ID 2544295
gaacagctttataagatcaaacgtgacaagttgaaggaaattgaaaggaaaagattagaactaatgcaga

SEQ ID NO: 217 - DNA, 80 bp, Artificial Sequence
  Affymetrix ID 2544306
agaataagcttatctatctggtacctgagaagcaattattaaatgaaagaattaaaaacatgcagttcagtaacacacct

SEQ ID NO: 218 - DNA, 147 bp, Artificial Sequence
  Affymetrix ID 2546279
catcagcaatctgcatgagaaggtaaattacaatgagaaacttaaaaagcttacggtctaagtcaaccaatgtagaaggtagtggttgaagaaaacaagctttgacttttaaaatttcctaaccaggggatttatttagaaattctg

SEQ ID NO: 219 - DNA, 90 bp, Artificial Sequence
  Affymetrix ID 2549323
acaacatttgacacggtctttggcaatcgagctgttggataaagtaaataatccagatcattccacttaccatgatttcgatgatgatga

SEQ ID NO: 220 - DNA, 130 bp, Artificial Sequence
  Affymetrix ID 2550840
ccacatgctaaatggcgcagctttaagaggtgaaattgaaacagtaaaacagttgcatgaagccatcgtgactctagggttagcagaaccatccaccaacataagtttcccattggtcactgtacacttg

SEQ ID NO: 221 - DNA, 98 bp, Artificial Sequence
  Affymetrix ID 2550842
gcaaaatatgaatccgacatggttactggtggctatgcagctttaataaatttatgctgtcgacatgataaagtagaagatgccttgaacttgaaaga

SEQ ID NO: 222 - DNA, 461 bp, Artificial Sequence
  Affymetrix ID 2550964
aacggttgtaccatactcagccatgccctttcattttgacgatagtgtttctaatattttgtatttttattcccctccccccatttttgtattacttaagatagattatcagaaagacagttactttgtcaaagagtatgggcacttgatacataatgccaaattattcttcataagagctgttgccaaatcagtgataatgttcatttaattgtattcttgccagccatgtttactggggtgatagttgttattgtggttgttattgttctttaggggtaggttcccaatatgtggtctttaaataattatctaatggtgtttaaaaagatgtttattctgtttgtcaggtacaaagatatttatgatacatgtatgacttgtctaagttattaacattttctctagccttaggtaatgcatgaaagcacatgtttcagtgccactcacataagaagtgc

SEQ ID NO: 223 - DNA, 138 bp, Artificial Sequence
  Affymetrix ID 2552021
agtatgtatgtacaagactcaagtaaatagaaaggcagctttcaatcacaaatcagtttttcagattttactgtggaagcatatttaatgcacacatttgaatgttacacataaataattttaacgatggagtccaag

SEQ ID NO: 224 - DNA, 34 bp, Artificial Sequence
  Affymetrix ID 2553312
agtcatgtgttgaaatagcggaattacttcaaca

SEQ ID NO: 225 - DNA, 50 bp, Artificial Sequence
  Affymetrix ID 2553583
tgtggaatgcattgtgaactgtaaaagcaaagtatcaataaagcttatag

SEQ ID NO: 226 - DNA, 129 bp, Artificial Sequence
  Affymetrix ID 2553585
ggagcactaccatctgttttcaacatgaaatgccacacacatagaactccaacatcaatttcattgcacagactgactgtagttaattttgtcacagaatctatggactgaatctaatgcttccaaaaa

SEQ ID NO: 227 - DNA, 40 bp, Artificial Sequence
  Affymetrix ID 2553595
ttcactcttcagtgttcctgttatttatgaacggcatcag

SEQ ID NO: 228 - DNA, 31 bp, Artificial Sequence
  Affymetrix ID 2553907
ttctgctccatattttgtatctgtttagtcc

SEQ ID NO: 229 - DNA, 28 bp, Artificial Sequence
  Affymetrix ID 2554001
agtttttacagattacgagcatgacaaa

SEQ ID NO: 230 - DNA, 36 bp, Artificial Sequence
  Affymetrix ID 2555279
tttttattgacatgactgattgattgattgctattc

SEQ ID NO: 231 - DNA, 60 bp, Artificial Sequence
  Affymetrix ID 2555281
cctgtgtaggaccattgaatctacaatccatgtcgtcacaaggatatctggcaaaggaaa

SEQ ID NO: 232 - DNA, 108 bp, Artificial Sequence
  Affymetrix ID 2555288
tctgaagcaaactgtgccaatttgatcagcactcttattacaaacttgataagccagtatcagaacctacagtctgatttctccaaccgagttgaaatttccaaagca

SEQ ID NO: 233 - DNA, 233 bp, Artificial Sequence
  Affymetrix ID 2555330
ggccatgatatgacttatagggaaaatacgtgttagacacatttcattcatatgagttttaatactattggcagtgagttaaacgttaatgaatcaacaatatatactaaataaggtatctttaacagaaacactcataaaacaaagtaaatgtatcgattggttgacaaaaatattaccagaggctcacagaaacttaatcgtgtatttgccctagtagtaataatttggta

SEQ ID NO: 234 - DNA, 52 bp, Artificial Sequence
  Affymetrix ID 2555409
gatgaagacgatgatcatggtcataatcctcccaaaagcagttgtggtacag

SEQ ID NO: 235 - DNA, 108 bp, Artificial Sequence
  Affymetrix ID 2555411
agcagtgataatagcgatacacatcaaagtggaggtagtgacattgaaatggatgagcaacttattaatagaaccaaacatgtgcaacaacgactttcagacacagag

SEQ ID NO: 236 - DNA, 58 bp, Artificial Sequence
  Affymetrix ID 2555416
ctggcttattagcaacaatgtggtggagcatatatttggaccaaatttacatattgag

SEQ ID NO: 237 - DNA, 113 bp, Artificial Sequence
  Affymetrix ID 2555507
gatgcagttctcattgattatcagagaaatgtcccagctgctagagaaccagaagtgcttagtactatggccataattgtcaacaagttagggggacatataacagctgaaat

SEQ ID NO: 238 - DNA, 61 bp, Artificial Sequence
  Affymetrix ID 2555522
cacttgtacatgcaaccttggaaacattgctcagatttctgaactggattcccctgggata

SEQ ID NO: 239 - DNA, 37 bp, Artificial Sequence
  Affymetrix ID 2556229
tgacaaaaatgcacgaagctatatttgatctccttcc

SEQ ID NO: 240 - DNA, 42 bp, Artificial Sequence
  Affymetrix ID 2558208
gtgtcctacttctcatttaagctactcaagaaagagtgccca

SEQ ID NO: 241 - DNA, 341 bp, Artificial Sequence
  Affymetrix ID 2558313
cccagaatacctagtttgaagcttatttttttttccattcacttgcaagctggattaagacattaactataataatatggaatgttcagcatttatgtttggtaaccagatgtatatggtttgtataaccatactttagaatttagttcagataaattggttttggtatactttgactacagaaagtgatttcaacataagatgtgatgaaggaagtctcccatgaaaaacttagtaaggatcaactgatgctatggacaaaacctaaaaaaagagatttgtagatgctttcaaggctttgactatgaaatatagaaaactggatgggcattcttactgac

SEQ ID NO: 242 - DNA, 85 bp, Artificial Sequence
  Affymetrix ID 2558519
tctaaagccagtggcttgaggctacagggagtgtagtaaagccgttgtttacttaaagatttatcaaatcagtcagtgcaaatgt

SEQ ID NO: 243 - DNA, 101 bp, Artificial Sequence
  Affymetrix ID 2559293
agaaatcctgtaagtacttggaagaatttatcaccaacatcactaatgtgcttccagagacagttcacactaccaagctctatggcaccacaacttttaag

SEQ ID NO: 244 - DNA, 25 bp, Artificial Sequence
  Affymetrix ID 2562334
cgtgcacatcacaaggacattttca

SEQ ID NO: 245 - DNA, 88 bp, Artificial Sequence
  Affymetrix ID 2565264
tcactctggatccagtgacagcaggtgtcatgggtcaagcataaatcatatatagcattttcaggcatgttcctggtagttcttttga

SEQ ID NO: 246 - DNA, 29 bp, Artificial Sequence
  Affymetrix ID 2575142
ggactgaagaaaaacttctctggagaaaa

SEQ ID NO: 247 - DNA, 460 bp, Artificial Sequence
  Affymetrix ID 2577959
ctgctagtgcacaggctgtactttaggtacttaaaatatgcactagaataaatttgcaaggccctaaaatatcactgttatttttggagtaattcagtataggttcgtttaaaagagatttttataacttcagacatgcatcagtaggaaataacttgagaaattcatatggttatgttacaaattcatattctgttactacagtaaacgttaagagttttaaacagttaagattgtacaatttttcttcttttctatattacaagggccccagtgttaatgtcttagattttcagtatttgaacttatttttttaaattctgtcattgagataagaataattcaggtagcatctgaaattttaatgaatgtataattggcatatcatggaaaattaaccagaaagtatcagttcttaaaagttatgcctagaaattatgtaaagctaaactactggtta

SEQ ID NO: 248 - DNA, 39 bp, Artificial Sequence
  Affymetrix ID 2581588
gataccagaataccattgttgctggaagtcttattacaa

SEQ ID NO: 249 - DNA, 49 bp, Artificial Sequence
  Affymetrix ID 2583054
ggaagaagcggcaaatgccaaattattggaggccgagaaacgaataaag

SEQ ID NO: 250 - DNA, 32 bp, Artificial Sequence
  Affymetrix ID 2583056
atggaaaaagaacttcgagctcagcaaattct

SEQ ID NO: 251 - DNA, 186 bp, Artificial Sequence
  Affymetrix ID 2583249
gggcaaactgttcaatcacttttgagaaattacatggttcctatttaggtttgaaaaagtagtttgttgccataacatcagaccttaaggtgttttaaagtgtatcctaagggtactgacagaagaaaaatacagtgttatggaagtatacacaactcatgaccacacagtatagatccattcatg

SEQ ID NO: 252 - DNA, 46 bp, Artificial Sequence
  Affymetrix ID 2583609
gttaggagaatgtgccttttttgtacctttgcatttaggttttata

SEQ ID NO: 253 - DNA, 39 bp, Artificial Sequence
  Affymetrix ID 2584845
ttcagtttttttcaacgcagtaagaaaaagcgagaccaa

SEQ ID NO: 254 - DNA, 29 bp, Artificial Sequence
  Affymetrix ID 2588838
agctggttgagactaccatggttccaagt

SEQ ID NO: 255 - DNA, 565 bp, Artificial Sequence
  Affymetrix ID 2590750
cttggttttacctgtagactgtggaactattttaccttaagacctgaaaaagttttgtggattataaatttctttcatacggttgtattttctgatcattggtttcttaatatggttgtactacagtatacttggttgatttaggttgcacattcactgaattcactgagattattcctataattttaaagtatcatttatttgaaaaacatacattatcaacatgtttttgatatttgataatgaaaaaaatctttgcttgtttatttctgaaaaagaactgtatttagtgattattttagatagtgatattatagtattcatctgtgtgtaaattatttcatatagggaagagttctgatctgtacctatggttcttattgaaaacaacattggatgtgcatttctgtgatgttatgaatacatttctactttattttgaaacatttgccaaactaaatactgtaacactgtataacatttaaaaatgttaaagaactgcttagtattagaagcagatcatttcccaaaattctaagagcagcagcatatgttgttgcttgta

SEQ ID NO: 256 - DNA, 36 bp, Artificial Sequence
  Affymetrix ID 2590802
attgctgaatactattgacgtttgccaagtcttctt

SEQ ID NO: 257 - DNA, 115 bp, Artificial Sequence
  Affymetrix ID 2591635
tctgcagtttctagcggggtttttacgagaaccatcaggactaatgaggctttctatttgtccattaacagacttgagtgaagtcataatctcatcggtgttgattttgaaatcc

SEQ ID NO: 258 - DNA, 731 bp, Artificial Sequence
  Affymetrix ID 2591638
cctgtggcatgccaatgaatctttctgatgggagacatgtacagattttgtgcatttatgttctgaatgcaagtcaacaattctgatctagagtttaaaagtgaaagtacattagcaccataacatgcgtctttaaagccttcccaaatattagtaatcttgaccagcaatgacaagaaaaaagaggagcacctttacaagcagttgatatccaatattaaaataattgtggctttaaaaatatttctttaaattcttgcattacacttttctttttaaaccaatcttccaggagattaatcaatgaaatttataagttttatcaacgtataaaatttttttcatcttctgggactcatagaatacaatctgtgtttctgaccagttgaggtagttaaaatagggagggcttttctaatttcgtatttgactatttcagaaagaaaggttatcttttactggtgagcacagtcattgctctgcagatgggctaggattcaaagaatataacacagtgttgttatcataaagagtgttgaagtttatttattatagcaccattgagacattttgaaattggaattggtaaaaaaataaaacaaaaagcatttgaattgtatttggtggaacagcaaaaaaagagaagtatcatttttctttgtcaaattatactgtttccaaacattttggaaataaataactggaattttgtcggtcacttgcactggttg

SEQ ID NO: 259 - DNA, 115 bp, Artificial Sequence
  Affymetrix ID 2593447
acgactgtaaacaactattggattctaaatgctcgacacataaatttaaccacactttctgtagctggaggattccgggattaccaagttcgttcaggattgacttttctaccaa

SEQ ID NO: 260 - DNA, 35 bp, Artificial Sequence
  Affymetrix ID 2593692
gtattctttatgctttccaagaacagactacagag

SEQ ID NO: 261 - DNA, 54 bp, Artificial Sequence
  Affymetrix ID 2593714
gaatgctaggacttacatggatgtaatgcgagaacaacacttgactaaagaaga

SEQ ID NO: 262 - DNA, 30 bp, Artificial Sequence
  Affymetrix ID 2593758
tcatctctgatgcaatgaaaaaagttggaa

SEQ ID NO: 263 - DNA, 66 bp, Artificial Sequence
  Affymetrix ID 2594506
cacacgatagtaaggagcatttagcaatgatggaaaggattcttggacctctaccaaaacatatga

SEQ ID NO: 264 - DNA, 31 bp, Artificial Sequence
  Affymetrix ID 2596691
taacctgcttttgtccgagaacatcttccca

SEQ ID NO: 265 - DNA, 257 bp, Artificial Sequence
  Affymetrix ID 2597013
ctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactac

SEQ ID NO: 266 - DNA, 35 bp, Artificial Sequence
  Affymetrix ID 2598267
gttctgcttcgaagtattcaataccgctcagtatt

SEQ ID NO: 267 - DNA, 34 bp, Artificial Sequence
  Affymetrix ID 2598289
cttcatggaccagagatcttggatgttccttcca

SEQ ID NO: 268 - DNA, 35 bp, Artificial Sequence
  Affymetrix ID 2598781
ttctgatggcggactttaccgtgacagcggaagtg

SEQ ID NO: 269 - DNA, 112 bp, Artificial Sequence
  Affymetrix ID 2599342
ccaaccgtatttctttagatacaggagttttgaactcaaatacttaggagaaaacaagttatgactgcattatcctgcaactcattaccagtaatatattgcaaagcgaaac

SEQ ID NO: 270 - DNA, 60 bp, Artificial Sequence
  Affymetrix ID 2599903
ctggccactgctttgtatcagggtttttcacgcttttgtagaactgaggtttcaataaac

SEQ ID NO: 271 - DNA, 107 bp, Artificial Sequence
  Affymetrix ID 2606647
acacgtggaaatcccttgaatttaggacctggttaaccagaaagataagactgttcttaacgacctagatgattctgttcatctctgaacgggatcaggttttgtcc

SEQ ID NO: 272 - DNA, 64 bp, Artificial Sequence
  Affymetrix ID 2607152
agctattcaatttctgttccgatcttcaaacagtttcacaagaatatcattgggaaaggaggcg

SEQ ID NO: 273 - DNA, 96 bp, Artificial Sequence
  Affymetrix ID 2607374
ctgatggcactctgttgtattttcttactgaagttcagtgataactctgagcagtttcattgtgatcactgtaaatggtaatcagttggaattctc

SEQ ID NO: 274 - DNA, 44 bp, Artificial Sequence
  Affymetrix ID 2609635
acttgcacatgaagtgcatatgttcatatttacccgtttggaga

SEQ ID NO: 275 - DNA, 151 bp, Artificial Sequence
  Affymetrix ID 2609657
gactgaccagtatgaagaagctttcactaatcagtacagtgcagatgtacagaacgcgcttgaacaacacctacattctagcaaggaatttgtgggcaaacctactattttagacactattaataagactgaattggcctgtaataacaca

SEQ ID NO: 276 - DNA, 134 bp, Artificial Sequence
  Affymetrix ID 2609668
tggagcagagactagacgtagaaaagcacgacggaaagagctagagatggagcagcagaatgaggcttcagaggagaataatgaccagcaatcacaagaagttccagaaaaagtaactgtatccagtgatcatg

SEQ ID NO: 277 - DNA, 31 bp, Artificial Sequence
  Affymetrix ID 2610353
tttttcatttcgaatcttgtgaatgtattaa

SEQ ID NO: 278 - DNA, 139 bp, Artificial Sequence
  Affymetrix ID 2611083
ggaatgcctcaaaagactactcagctgatccaaacatagtaacttcccaaacacaagactcttgacgtaggcctcagaatttggggaaaaaaattcctgcattgtttaaaaatacagtgtccttgtaaagcagaatagg

SEQ ID NO: 279 - DNA, 97 bp, Artificial Sequence
  Affymetrix ID 2614100
tgctggtcccaaaggcgataacatctatgaatggagatcaaccattctagggcctccaggatccgtgtatgagggtggtgtattctttctcgatatc

SEQ ID NO: 280 - DNA, 35 bp, Artificial Sequence
  Affymetrix ID 2614135
tttcaagtgtgagcttagacgtcaaccctaaaata

SEQ ID NO: 281 - DNA, 129 bp, Artificial Sequence
  Affymetrix ID 2614137
gactcagttaattgatttccaggaagtactcatagcaagttcataaaagttcttgagacctaaatttcttcacaaaaaaagaaaagatcttaagtcatacattttaattgtgtagaggttgttcaactg

SEQ ID NO: 282 - DNA, 104 bp, Artificial Sequence
  Affymetrix ID 2615656
gagtgtaacgaagtctggatttctgataccttgtcatttgggggattttattttactttgttgctttaaaattcaatgcagagaagttgttgactgtaggggaa

SEQ ID NO: 283 - DNA, 30 bp, Artificial Sequence
  Affymetrix ID 2616071
ctggccattttagttgtatcacagcagaat

SEQ ID NO: 284 - DNA, 999 bp, Artificial Sequence
  Affymetrix ID 2616248
tttcactgggacaaactcacgttcaatgccactcagtataatttcaagtctgataagcatctaagtatttttaccccgcttctaaaacctgatgaggaattcaaaataagcacacagcattaaatgacatttattgttccataaatcttgagacccaaaaaggaatgctaaatagacaagcaaaacttttaaaacaaacgagataaactcacttctttccccagtgactggtacagaaaacatgtggtcacacgaaagcaaagggaaaaagtcagaaaggaaaactctctgcctataggatctataggagttacagatattttcaaatcgatgatgaaaatagatcgtgcttctttgtagcaaataattaaccccctttatgaataaaacataaaatgtcaaagcttttactcactgaagtagtttgtcttctgggagagatttcaaactcaaaattactcatttcctattttttgcctgaacacaatgaggatcaaatagtacattcaaatcaggacacctgggtatatggacaaagatatcctacaattaaaatggaatagaagttaaaaaaaaaattcagtcccctacaccacaggcctcaaacatgcttgctgatggattctgggaaggttttggatgaaggacggagatatgggaggaaagtgagaaaacagtggattccctttgaaaagtatgctagcagacaagcatttggttttatgaaagaggcactcttatagagaaagaagctagtatgtggtgtataaaaagcccctaaatcatcaccagaatgtctatccatgattgtactaaagctcaatatttgagaggcttaagggatttgtatggtacattttagtgtttgattgataacatctagtgtttccagaaaaaaattcatcttgttcataatgaagatcccttaacacctagtcagaagctatcaacaataatactttctccccctacttgggtcacctatttccaatgtctggg

SEQ ID NO: 285 - DNA, 57 bp, Artificial Sequence
  Affymetrix ID 2617578
agaaaagccgatatttgccattcttcctatatcctggaatatatcttgcatcctgag

SEQ ID NO: 286 - DNA, 30 bp, Artificial Sequence
  Affymetrix ID 2618656
ggaggcagattgatagtaggattataataa

SEQ ID NO: 287 - DNA, 95 bp, Artificial Sequence
  Affymetrix ID 2619365
tgttagaacatgagtctgcgcagccatagagtagggatctgcagaccctgcaagaacagttcctgtcactggccactgtcagtggtcattaataa

SEQ ID NO: 288 - DNA, 32 bp, Artificial Sequence
  Affymetrix ID 2625632
gaaactgctgcacgagtaaatcaatcagctct

SEQ ID NO: 289 - DNA, 51 bp, Artificial Sequence
  Affymetrix ID 2625643
atatgtgattctgttggactggcaaaacagatagctttgcatgctgaactg

SEQ ID NO: 290 - DNA, 30 bp, Artificial Sequence
  Affymetrix ID 2625893
cgtgacgtgcatgatttaataaaataactt

SEQ ID NO: 291 - DNA, 27 bp, Artificial Sequence
  Affymetrix ID 2627951
gttatcatcagactactgaaggacttg

SEQ ID NO: 292 - DNA, 35 bp, Artificial Sequence
  Affymetrix ID 2633534
gaaactgtccttttgacaactctcttatataataa

SEQ ID NO: 293 - DNA, 57 bp, Artificial Sequence
  Affymetrix ID 2633651
tgtgtcacagaataggacgatatgattctacaacataatcaactccctattaaattc

SEQ ID NO: 294 - DNA, 177 bp, Artificial Sequence
  Affymetrix ID 2638377
gaaagatggtccagtacatgacatgggtgagagtcatattttttccctacccatttaatgcaagatataaagaagctttcctagcttctatctcttttagaattttctgttaaaaattcttactctgtcttatgatgaacagattgatgaagattgtggcctaccattaagtgtaaa

SEQ ID NO: 295 - DNA, 34 bp, Artificial Sequence
  Affymetrix ID 2638451
aaaattggatcgaacatttcacctctcatattaa

SEQ ID NO: 296 - DNA, 41 bp, Artificial Sequence
  Affymetrix ID 2638452
tgatgactatatgtattcctgcctaaataaatcatctatta

SEQ ID NO: 297 - DNA, 70 bp, Artificial Sequence
  Affymetrix ID 2641575
acattctcaactcctcttgaatctatcccccaagaaaccatcttatccctgtaataaatcagcatgtatt

SEQ ID NO: 298 - DNA, 155 bp, Artificial Sequence
  Affymetrix ID 2643579
catctgaagtagcagctctttaaaaacatgaagagataaaattataaaaatgatacatctaaagcagtggtgaagaaagctgaaaaactgatacttttgataggcattttctctgcactggtttgtttaaaggacttcttccagcaataagttga

SEQ ID NO: 299 - DNA, 56 bp, Artificial Sequence
  Affymetrix ID 2645294
aatatttcataagtatggatgtgcaatattactggccatagtgcaaaaggtgaaag

SEQ ID NO: 300 - DNA, 113 bp, Artificial Sequence
  Affymetrix ID 2646066
tccgccttctatgatggaacatacgcttcccccacctccatccggactgccttttaatgcgcagcctagagagcggttaaaaaaccctaatgctcctatgttaccgccaccta

SEQ ID NO: 301 - DNA, 42 bp, Artificial Sequence
  Affymetrix ID 2646085
ctgttacaacttctaaatgggaattatttgaccagcatgaag

SEQ ID NO: 302 - DNA, 326 bp, Artificial Sequence
  Affymetrix ID 2647361
ttgatctgatcgatttggctgcatactttcggtacgtataacattctaaacttaaaatagaaatttttatattacaaaacgtagaagtaaaattttaaaaagttaaagtactagcacatatatgtgttaggaaaatggtctctgtcaattgcccattttcccaattaaattaacctacgatttcctttttttaacagcttatttttttcataaaagttgtactttgagaagttactttctaattacgtcatgagaacacaacttgtaattagcaacacttctgtcagtctagatcacttcttctgcagagagcttttcaaccaagt

SEQ ID NO: 303 - DNA, 48 bp, Artificial Sequence
  Affymetrix ID 2647678
tagggacagtagatactgataacgcaaaaataatccaaccctttctgc

SEQ ID NO: 304 - DNA, 34 bp, Artificial Sequence
  Affymetrix ID 2647775
accacaatcaggaaacgataagccattatcaaaa

SEQ ID NO: 305 - DNA, 29 bp, Artificial Sequence
  Affymetrix ID 2647779
actgaaagcaatcgaacaactgaaagaac

SEQ ID NO: 306 - DNA, 32 bp, Artificial Sequence
  Affymetrix ID 2648202
atacccataatatctgccgaacatctgactag

SEQ ID NO: 307 - DNA, 273 bp, Artificial Sequence
  Affymetrix ID 2651521
agggtgattaggaattaactggacaaagaagagggaaagtctttgcaagtagaggaaagaatctgcttggagctcagataactattatttgaaaacataatgacatctagttcaaacttgtgactgagttccacagtagaattcacagaaaaaaaattattaaatataatatttccatcagtctgtgtctaaaagattaaaaaagagcaaataacaatcttaataaactgatgatagattatagcctcatctcttccaacatccgattctgtg

SEQ ID NO: 308 - DNA, 85 bp, Artificial Sequence
  Affymetrix ID 2652021
acatgacatactgtgctctgagcttatacctcaattgtattttgtgctgttttccattttcatgccttgtaaataacttgtatag

SEQ ID NO: 309 - DNA, 122 bp, Artificial Sequence
  Affymetrix ID 2654335
caggttctgtgaggcattctacctcaccagcaagctcagaatactcttggaagtcagttgagaaatacaaaaaatacgctcactctggatcacgtgatttcagtagacatgagcaaagatac

SEQ ID NO: 310 - DNA, 112 bp, Artificial Sequence
  Affymetrix ID 2654874
acattgtatgacagcgtgtacctgactttatacaatatttgttttacttccctacctattctgatatatagtcttttggaacagcatgtagaccctcatgtgttacaaaata

SEQ ID NO: 311 - DNA, 28 bp, Artificial Sequence
  Affymetrix ID 2654878
cagtcatggttattacagtcacagtaaa

SEQ ID NO: 312 - DNA, 203 bp, Artificial Sequence
  Affymetrix ID 2655475
tctactttaattcattggagcatgggatttggagttaggtggttttgggtttgaattccagctctactatttttggttgtgtgatagagttatttaacctctctgagcctcagttccctcgtatgtaaaatgatgataataatacctacctcacagggttgttgtgaggatttaaattagatattgtacgaaaagtgcctagc

SEQ ID NO: 313 - DNA, 25 bp, Artificial Sequence
  Affymetrix ID 2655509
ctgctttgggatagtgtgagcttca

SEQ ID NO: 314 - DNA, 42 bp, Artificial Sequence
  Affymetrix ID 2655678
agcagctgctccacatttgtagcgaacactttgactccaaag

SEQ ID NO: 315 - DNA, 85 bp, Artificial Sequence
  Affymetrix ID 2656791
gttgctaagccccagcaagcaatcctaggtagggtttaatccccagtaaaattgccatattgcacatgtcttaatgaagtttgaa

SEQ ID NO: 316 - DNA, 107 bp, Artificial Sequence
  Affymetrix ID 2658354
gtttcacgaagcatttatcattcacatcatcctaccttaaagcttcaacgaccccaattaaggacatcctttcagcagttctcttctctgacaaaccttcctttacg

SEQ ID NO: 317 - DNA, 121 bp, Artificial Sequence
  Affymetrix ID 2658632
actttttgtagagagagctgtattaagtgactgaccatgcactatatttgtatatattttatatgttcatattggattgcgcctttgtattataaaagctcagatgacatttcgtttttta

SEQ ID NO: 318 - DNA, 61 bp, Artificial Sequence
  Affymetrix ID 2663553
ccttgcatattacttgagcttaaactgacaacctggatgtaaataggagcctttctactgg

SEQ ID NO: 319 - DNA, 31 bp, Artificial Sequence
  Affymetrix ID 2666503
tgattcaaatgttagtccagagaggttatga

SEQ ID NO: 320 - DNA, 93 bp, Artificial Sequence
  Affymetrix ID 2666519
gattgggtactagtacagctaaagaagcaaaggaatattttgctgatatggaaaggcatcgcatcttgtttagatatgctggtcctgaagatg

SEQ ID NO: 321 - DNA, 55 bp, Artificial Sequence
  Affymetrix ID 2666524
aaaagttacgatgatgcagaatctctgaaaaccttacgctatggaaagattatga

SEQ ID NO: 322 - DNA, 72 bp, Artificial Sequence
  Affymetrix ID 2669316
ccatcttcaagtttggactcatagacttgggttaaagattttactttttgctccatttcactattttgtttt

SEQ ID NO: 323 - DNA, 67 bp, Artificial Sequence
  Affymetrix ID 2669571
aagaatggcaaatatcggcttttctcagatatctttgaaattattttaatgcgtacaaaacatgtag

SEQ ID NO: 324 - DNA, 32 bp, Artificial Sequence
  Affymetrix ID 2670438
aaattatggtgacttgtgacacttcatcaatg

SEQ ID NO: 325 - DNA, 28 bp, Artificial Sequence
  Affymetrix ID 2672775
aatcatggctggaataaagtaagtttcc

SEQ ID NO: 326 - DNA, 31 bp, Artificial Sequence
  Affymetrix ID 2674243
ttccaactactaatagaataaaggcagtttt

SEQ ID NO: 327 - DNA, 41 bp, Artificial Sequence
  Affymetrix ID 2675269
ggaggcttgtcatactgtctagaggttgactcttagttctg

SEQ ID NO: 328 - DNA, 66 bp, Artificial Sequence
  Affymetrix ID 2675802
tagctgggggagtggcatggggcaggggcccagttctcagcagcagacactctgtacagttttttc

SEQ ID NO: 329 - DNA, 47 bp, Artificial Sequence
  Affymetrix ID 2676931
ttttaggttgcagaatggactcctcatctttatattcacgaaaaagc

SEQ ID NO: 330 - DNA, 45 bp, Artificial Sequence
  Affymetrix ID 2677655
tatttaaatcatgatggcctgtaacagttgaagcatctaaaaatt

SEQ ID NO: 331 - DNA, 43 bp, Artificial Sequence
  Affymetrix ID 2677695
gtaggttccaggatccgttgtaatgttgtgatctcatacctgt

SEQ ID NO: 332 - DNA, 29 bp, Artificial Sequence
  Affymetrix ID 2680511
ccagagtttcaaacaagactgtacaagaa

SEQ ID NO: 333 - DNA, 252 bp, Artificial Sequence
  Affymetrix ID 2682663
tatataccgcttcacaaaggcagtgaacacattacattctacaaaatctactttacagaaatttaaaaactttaaatatcaaaaggtacagctgaagaaacaggtataaatttggcagccagtaattttgacagggaagttacagcttgcatgactttaaatatgtaaatttgaaaatactgaatttcgagtaatcattgtgctttgtgttgatctgaaaaatataacactggctgtcgaagaagcatgttc

SEQ ID NO: 334 - DNA, 109 bp, Artificial Sequence
  Affymetrix ID 2685954
ctcatacatatcattatcgtggaacacgttgaatgtttgtgattctgtgtggcctttttggggctggaaaatgtatgaattcttcaactgtcttacaagagatctgcta

SEQ ID NO: 335 - DNA, 132 bp, Artificial Sequence
  Affymetrix ID 2691756
gtgagggaatagctccaattaaaatgaaagtatttcttgaagatacagggcaagattttcccttaatgccaaatgaagagagcagtcttccagcagttgaaaaagaacaggcgagcactgaacatcaaagta

SEQ ID NO: 336 - DNA, 220 bp, Artificial Sequence
  Affymetrix ID 2692111
gggacatggataatcacataatttgctattattactttaatctgacttgaagaaccgtttaaaaataaaatttagcatgtcatatattccttatagtaggcttatttcaccttttttctgtccagagagagtatcagtgagaaagattttaagggtgaaaacatgaattggtgggacttagtaaagagctgaaaaactactacaatgtgggctacggtta

SEQ ID NO: 337 - DNA, 36 bp, Artificial Sequence
  Affymetrix ID 2694407
atgaacaagtgatagattctctgactgtgaagatca

SEQ ID NO: 338 - DNA, 65 bp, Artificial Sequence
  Affymetrix ID 2694648
tgtttgtttcaaattttcattaatgtatatagtgcaaaataccatattaaagaggggaatgtgga

SEQ ID NO: 339 - DNA, 261 bp, Artificial Sequence
  Affymetrix ID 2694649
tcacagtgttgatgttggacacatagatggtgatggcaaaggtttagaacacattattttcaaagactaaatctaaaacccagagtaaacatcaatgctcagagttagcataatttggagctattcaggaattgcagagaaatgcattttcacagaaatcaagatgttatttttgtatactatatcacttagacaactgtgtttcatttgctgtaatcagtttttaaaagtcagatggaaagagcaactgaagtcctagaa

SEQ ID NO: 340 - DNA, 97 bp, Artificial Sequence
  Affymetrix ID 2695413
gctgaaattggtgttgggcatattagcaaaatattcttaatttgtggactcgattcttttttactacatatttcccaagttatcttaagatgtctgt

SEQ ID NO: 341 - DNA, 33 bp, Artificial Sequence
  Affymetrix ID 2700507
tatcataaccttgaatggcgactagatgtacag

SEQ ID NO: 342 - DNA, 64 bp, Artificial Sequence
  Affymetrix ID 2700829
catatgcttgctatttaaagctgtgtgttggttttgtttttctgccacattcactagtttttta

SEQ ID NO: 343 - DNA, 264 bp, Artificial Sequence
  Affymetrix ID 2701289
tggtactagccttccgttttcaacagattaaaatagccaatatttatgtattagcaaacttttttgtcaagtttcgtgctgttcagattaagctatttcctcaagtcaaaaatacaactaatcttttgatagagtaaatattgtttttaaaaaaatccatatgttgaccttcaaagttaaattcagtgtaagttttagaattaacagatatcacaattaaagggctaagatttcaaatttcctgataaggtactccagcaaagc

SEQ ID NO: 344 - DNA, 127 bp, Artificial Sequence
  Affymetrix ID 2702329
ttattgctataagaactggagcgtgaattctgtaaaaatgtatcttatttttatacagataaaattgcagacactgttctatttaagtggttatttgtttaaatgatggtgaatactttcttaacac

SEQ ID NO: 345 - DNA, 31 bp, Artificial Sequence
  Affymetrix ID 2703240
tgtgcaagtagtactcgatggactaagtaat

SEQ ID NO: 346 - DNA, 123 bp, Artificial Sequence
  Affymetrix ID 2704511
gaagtggtcagtgaagtaattgtggaattaatggccaaagaagagaaatggctgaatggagctgaccttgtgaagaagacaaggaaggaggaagctctggatgttctagcaaagtgtagctaa

SEQ ID NO: 347 - DNA, 216 bp, Artificial Sequence
  Affymetrix ID 2704898
agcaaactatttgaacacttacatgtgcagtgtttggtgaacatgacatgaggaactagtagtaagtaaaatcttccccccaaaattcattgtggcttaaataaatatgaacataatcattactacttaatatactgagagggaatcttaataaacttggaactgggagggaatatttgtatacattgggtaaagggttaggctagatgacatcta

SEQ ID NO: 348 - DNA, 70 bp, Artificial Sequence
  Affymetrix ID 2704935
caagtttgtccagtggaagaccatctacatctcccacaggaagtgtcacacagcagtcaagtatgtccca

SEQ ID NO: 349 - DNA, 180 bp, Artificial Sequence
  Affymetrix ID 2705001
agcagaaaggttccaagtgaattagaaatataaatgaaattataggtgtttgagtttgtcttccaaaaagctgagctgtgacacaaataattcaaaattaaaataaaactatatcataaatatctgatttttccttctccagatctaaatagattgtattgggagaatatgtcattttaa

SEQ ID NO: 350 - DNA, 490 bp, Artificial Sequence
  Affymetrix ID 2706322
tgtctccgtgtaggaaatcccataagttcttatatttgttccagttcccatttcctgccattgaccagataacatcattgactttcaaatgacttttagaagtgataactcttaatttcctaatagatactagattgtattgaattctgttttaattattctctaggtaagtatgttttaggattaaataccttttacagatactgaaagtgcctccttttgtggtgtaaaaaacaaattatggtgcaaaaagtaatcactagattgaaatacatgaaggttttttgctttttgacatacgaaaatgtcaagagaaaggccaaagatttgtactttttcacttacaaagcactcctttttcccttaaacttctttctgtcaaattagatttaatgagagagtactatttttaaggagctatctgtttatgtagaatgattttgttaagagtaatgtaaactattattgagtagaggcctaaagaggactg

SEQ ID NO: 351 - DNA, 93 bp, Artificial Sequence
  Affymetrix ID 2706324
cgtaggaaacctaatatgctgagtatctggcacttgaattcctgcttttattgctggaggtccacatctgtggttgacctctgttattgttta

SEQ ID NO: 352 - DNA, 29 bp, Artificial Sequence
  Affymetrix ID 2707780
gacttcagtacgatgattgcagatgacat

SEQ ID NO: 353 - DNA, 31 bp, Artificial Sequence
  Affymetrix ID 2708798
gcaccacgcaagaagattcatcgcagagtcc

SEQ ID NO: 354 - DNA, 60 bp, Artificial Sequence
  Affymetrix ID 2709489
catttgcaactcatcagcctttgtgcaactgtgatgcagcagttatctcagaattgttaa

SEQ ID NO: 355 - DNA, 212 bp, Artificial Sequence
  Affymetrix ID 2710214
cctattttgatatgttctgcagagcccatgatatagtggatttttttccttttccatgtctctcttgttacacagaattaccaaatatttatattcctgatacttttcaaaatcagtatatttttacaaactttctgttatagactctgatttttctactatttatgtatttaaaccacaacaattatgatattatatcagcagacagtatg

SEQ ID NO: 356 - DNA, 93 bp, Artificial Sequence
  Affymetrix ID 2710217
caggggtatggaacatgctgtcatatttcattcataacacacatgtactatagctctaggcaacagatggacaatcgcttgtttgaactacaa

SEQ ID NO: 357 - DNA, 159 bp, Artificial Sequence
  Affymetrix ID 2711689
gttccagtgacaaagactaatttgccaaatccttcagtggatgtgaaaggaataggagatgaattatataatccagaaacacataaacgacatactttgttttgtgggacaactgttattcagactcgtttctacactggagaactcgtcaaagccata

SEQ ID NO: 358 - DNA, 42 bp, Artificial Sequence
  Affymetrix ID 2712424
tcagtacctgcttcttccagagaaataaagttagtttctatt

SEQ ID NO: 359 - DNA, 62 bp, Artificial Sequence
  Affymetrix ID 2713075
cataactcagatttctacaggagacagtggttttattcggattgtcttctgtaataggtttc

SEQ ID NO: 360 - DNA, 105 bp, Artificial Sequence
  Affymetrix ID 2713262
aatgactgtatattacgagtaaatgaagtagatgttcgtgatgtaacacatagcaaagcagttgaagcgttgaaagaagcagggtctattgtacgcttgtatgta

SEQ ID NO: 361 - DNA, 32 bp, Artificial Sequence
  Affymetrix ID 2713269
cccagtactggtcaacacagatagcttggaaa

SEQ ID NO: 362 - DNA, 111 bp, Artificial Sequence
  Affymetrix ID 2714106
aataatacatgcttaaagtctgctgttattctaaaatgaaagatatgaattcaacaaagttgatggataactttctttgactgctctacctgaatttagactaagcagtaa

SEQ ID NO: 363 - DNA, 31 bp, Artificial Sequence
  Affymetrix ID 2715470
tctgctagtgttacagatggaaataaaacca

SEQ ID NO: 364 - DNA, 31 bp, Artificial Sequence
  Affymetrix ID 2715717
ttctccagtgtaacatgttttactcacaaat

SEQ ID NO: 365 - DNA, 28 bp, Artificial Sequence
  Affymetrix ID 2720693
attaatatcggttacactgctgatgttt

SEQ ID NO: 366 - DNA, 1391 bp, Artificial Sequence
  Affymetrix ID 2724050
ccgccctgtgaaccagctgtaaagaaatgtagttaagaaatgcaaagaaatgtgttatcttggcacttgggtttgattttcctttttttaaagcactgcaaaacttctttaggatactaaaacatcctaattgggctgtttttttgttttgttttgtttttcagctaaaggcaaagataatttttttttcagctgaagtttttcttttttgcaactatgacatgaattgagtgataaaatggctctttgaaaagtagatattgtctgtattgtaaatgcttagtcatattcatggcaaagtgttcatttcttgagatggttctttcacaacacacaagaatcaagatggaacgcaaggaaaatctttttgttttgaccgaggtaaccagtttatgtgtgttccaatccatagagaagtgggggaaagggcgagaaaggatcagattgatctgggatcagctttgagcctgagttcagtgaatagcctcatgggcatctcatggaattgatgcatttgctgtggcatatttaaattttgttcaaaagatttgaagcaagaccttgcaaagactcttaacattactgttcagcttgcttagattgtgttaacagttcatttaacaaagatccgttttaactctgaacaaaagcaacccagagcggccattctctattcccatgctggtcaatacgctttcattaccaattggcccttaccaaacttttcttatatatatatttgtattttactatgatagttgagaaatttagcctcttagtcatttttagctgttattgtggaagacctcaaatacaagattagatgcccttgaacatgttttaatgatgtgtgtcatgttactatcaatggtgatttcaatcgcaatattttaaattgatgagaatgatttgtaaacatgaagttactattacgtaaattctgtttgttatagagtttcttcagttgttacccaagtgtcatcctagagaagtcagaagaatcagaatccatcgtattttagagttatgtgaatctacatcataaatgggcattaacattctaaattgcttggtttggagaatgtgttagcagcatagctatattcaactagggagatgctaaatacacagaagtttcaagagccttggaacagaattacaggggaactatatatgtatatgtatattttattaaacacccatctgcactatcagtattgcactaatgtggaatttgaaagactattttgctgatactgtatatatgcgcatcattcctgattagattgttgtaaagacaatctgaaagatctaaggttttaaaataatttggtttgaaaatatacagttgtcttgaaggaattgctgtcatacatgaagttgctccgagc

SEQ ID NO: 367 - DNA, 31 bp, Artificial Sequence
  Affymetrix ID 2724565
ttcatggcagctattccacagactcagaacc

SEQ ID NO: 368 - DNA, 144 bp, Artificial Sequence
  Affymetrix ID 2726514
gacctgatcccaaccttgaagaaagtcctaaaagaaaaaatattacatatgaggaattaaggaataagaacagagagtcatatgaagtatctttaacacaaaagactgacccctcagtcaggcctatgcatgaaagagtgccaa

SEQ ID NO: 369 - DNA, 38 bp, Artificial Sequence
  Affymetrix ID 2727970
gatttttgaggattatgtgactagtaataaatgtgaaa

SEQ ID NO: 370 - DNA, 109 bp, Artificial Sequence
  Affymetrix ID 2730701
tgtggcaaaaactatactcaaacgcctctttagggtttatgctcacatttatcatcagcattttgaccctgtgatccagcttcaggaggaagcacatctaaatacatct

SEQ ID NO: 371 - DNA, 113 bp, Artificial Sequence
  Affymetrix ID 2731872
ctaaggctatttataagtccagtgaagaagataaaaaagaagaagaggtgaaaaaaacattagaacagcatgacaatattgtgactcactacaaaaatatgattcgagagcag

SEQ ID NO: 372 - DNA, 180 bp, Artificial Sequence
  Affymetrix ID 2732403
atttgtcttgcattggagtctgttcttttttgctggctgctagaatagttgaagaagactgcaatattccatccactcatgatgtgatccggattagtcagtgtaaatgtactgcttctgacataaaacggatggaaaaaataatttcagaaaaattgcactatgaattggaagctacta

SEQ ID NO: 373 - DNA, 133 bp, Artificial Sequence
  Affymetrix ID 2735017
tacacgtaaaccacaaaagagtagcattccattttcttgaagtgcacatgatattatgaacaatacaaatgcattatttttatcattaatagtttaatcattaattatctcataagtcaatgcagagagtgaa

SEQ ID NO: 374 - DNA, 123 bp, Artificial Sequence
  Affymetrix ID 2736295
aaataaccgtttgctgctcacaggcacacctgtacagaacaatctgttagaactcatgtcgctgttgaattttgttatgccacacatgtttagtagtagcaccagtgaaatacgaagaatgtt

SEQ ID NO: 375 - DNA, 123 bp, Artificial Sequence
  Affymetrix ID 2738963
aagagacaagtttatcacagaattttttttcctgctggcctattgctataccaatgatgttgagtggcattttctttttagtttttcattaaaatatattccatatctacaactataatatca

SEQ ID NO: 376 - DNA, 763 bp, Artificial Sequence
  Affymetrix ID 2743194
ttacagtatctgcagaaagagtcccttccaaggcctcacacattcagacacatccatactgacacccactccacagccccatgcacccacaccagtgagatgtggagggtcagactcctaaaattaggcagctgttggggataagagttgatttgtttttcaatttttttgaaaacagaaaagcatgggggaatgcatttggccattacaatgctaattgagtttgtgtatattacatatatggcagttaacactgtaatattccttttacattctatatacacagaatgatatcaaggttttatggtcaacagaatattccaacttcagtcttaatgctgcttgtagtgatttctgaattcattataggggctttccctaaaaataattcaagtctatgttaagtgaaataaggcacaattaatattgatttgatttagggaaagggaaggaaagagaagggataaaaactggtttcaaatgatctttctgttggggaactagctctgacttaaacccacctgaaattccttctcctaatttccaaagatttctttataaagatatttttcttttccctgacaaagccaaaaagaaaagtttgggaattcacattttaatgtttcagtagcctgaacaactcaaattgatgtgtacccccacctcccctgatcaggtcgcctccctcacccatgttaaaataaaaacacagtatgtgacacaggagtcccttgatgtttcttgaggagatctgtacacttga

SEQ ID NO: 377 - DNA, 92 bp, Artificial Sequence
  Affymetrix ID 2744632
cccttagatcatatgtggacttattaaaactagaagatgtacttcgacagcatccattttacttcaaggcagcaagaattgctatagagatc

SEQ ID NO: 378 - DNA, 80 bp, Artificial Sequence
  Affymetrix ID 2745687
gctctccaagatgggcgaaagttcacttagaaactttacaatggatacagagtcaagtgtttataacttcgaaggagaag

SEQ ID NO: 379 - DNA, 269 bp, Artificial Sequence
  Affymetrix ID 2754300
aaaatgtgccacaaggatgtaataacaactgctgataaacaagaaaaggaatcttaaaattataaatttgctgtagaaaagataaaaaacaattatattttatttagaattttaccttgaataaaatattcccctgtataaaaaataaaaaagcttgctctggttagaattagagtatttttgtcttcaaatctgggaatttgcataatatttccatgatactttttcctttgtaccgcgtggcattcaagcatagcagattagaagga

SEQ ID NO: 380 - DNA, 37 bp, Artificial Sequence
  Affymetrix ID 2757037
aatcatgtttgtgactgtaaccatttgtatgaattat

SEQ ID NO: 381 - DNA, 155 bp, Artificial Sequence
  Affymetrix ID 2763813
ctcatactggaaaagtcgatcaatgttttgcagtttattgaaagtagttctatatataacaatgttataagcatttctttagaaatggttgaaaatgcttctaaaatgtgattatcgaccatggtatgcatgatcgttgtaattgttgacattcc

SEQ ID NO: 382 - DNA, 79 bp, Artificial Sequence
  Affymetrix ID 2766634
gtcttttacacatcctacctcgttccactctgcagagacatatgagtccttgttacagtgcctaagaatggaggatgac

SEQ ID NO: 383 - DNA, 32 bp, Artificial Sequence
  Affymetrix ID 2770195
tattttaccattatacatcatgtggacttatt

SEQ ID NO: 384 - DNA, 78 bp, Artificial Sequence
  Affymetrix ID 2770473
tccagaatattattagtctgcatggttaaaagtagtcatggataactacattacctgttcttgcctaataagtttctt

SEQ ID NO: 385 - DNA, 70 bp, Artificial Sequence
  Affymetrix ID 2771759
tacagatcctgacttgatagagaaatccaacttaaatagacagttcctatttcgtcctcatcacatacag

SEQ ID NO: 386 - DNA, 35 bp, Artificial Sequence
  Affymetrix ID 2773129
gttgtccagatatttatcctgaactcagtgtttga

SEQ ID NO: 387 - DNA, 207 bp, Artificial Sequence
  Affymetrix ID 2774054
ctcttgaagaggcaattcattaaactctcaaattttctatagaatcaagatagaacctttagatactccaactcaccaaaatgtaaaaaaactaacaaaaatatttggtcttcaataatgctaaatatctacatttttagaatttatcaacatttaactagataattgggcatgtcttaattatgcatgtacttatccatactaata

SEQ ID NO: 388 - DNA, 69 bp, Artificial Sequence
  Affymetrix ID 2775592
atgaacgagtacagcaatatagaggaattcgcagagggatccaagatcaacgcgagcaagaatcagcag

SEQ ID NO: 389 - DNA, 28 bp, Artificial Sequence
  Affymetrix ID 2775758
tcagtgtcaatcagattaaagtgtgtaa

SEQ ID NO: 390 - DNA, 31 bp, Artificial Sequence
  Affymetrix ID 2776094
tctttgtcaggcattacggaccttttttcca

SEQ ID NO: 391 - DNA, 28 bp, Artificial Sequence
  Affymetrix ID 2777492
tttttaagctacaaataaatgagatgct

SEQ ID NO: 392 - DNA, 25 bp, Artificial Sequence
  Affymetrix ID 2779488
cactctggtggataagttcaataaa

SEQ ID NO: 393 - DNA, 35 bp, Artificial Sequence
  Affymetrix ID 2779639
aggcaactatggcatagaaataaacatttgacatt

SEQ ID NO: 394 - DNA, 132 bp, Artificial Sequence
  Affymetrix ID 2779995
tgaggctgtgttgaagtgacttatctatgtgggatattgagtatccattgaaatggatttgttcagccatttacattaatgagcatttaaatgcaacagatatcatttcaggtgacttaacatgaatgaata

SEQ ID NO: 395 - DNA, 67 bp, Artificial Sequence
  Affymetrix ID 2780545
gattcccatctctaaggactccaagtgctagagacaagggggtctatgagcatttactgacttcctg

SEQ ID NO: 396 - DNA, 61 bp, Artificial Sequence
  Affymetrix ID 2783475
cactgagtttaccaaatcaactgtgtgaactgtttctgcactgcttgctaatggtttcatc

SEQ ID NO: 397 - DNA, 58 bp, Artificial Sequence
  Affymetrix ID 2789554
tctcgtctggcaatcttccaacaaatgtggcacctttaatcgtctttgatatttctga

SEQ ID NO: 398 - DNA, 42 bp, Artificial Sequence
  Affymetrix ID 2790573
aggagaaattcacttgcttcaatcattgctgcttcatatttt

SEQ ID NO: 399 - DNA, 37 bp, Artificial Sequence
  Affymetrix ID 2797449
aactacgtcaaggaagctagaccgagaacagcaagat

SEQ ID NO: 400 - DNA, 29 bp, Artificial Sequence
  Affymetrix ID 2801338
tattgtattttacttcagctatgggtgca

SEQ ID NO: 401 - DNA, 67 bp, Artificial Sequence
  Affymetrix ID 2801556
tcacttgttcaaagctgtgtaatcgtgggggtaccatctcaactgcttttgtattcattgtattaaa

SEQ ID NO: 402 - DNA, 91 bp, Artificial Sequence
  Affymetrix ID 2801679
gtaccttgtgggtcaacgactcgtgaactacgaacggaaatctggcaaacaaggctcatctccaccacctccacagtcatcccaagaataa

SEQ ID NO: 403 - DNA, 36 bp, Artificial Sequence
  Affymetrix ID 2802506
cctgcaggtgactgtcaacgtgatcaaggaagggga

SEQ ID NO: 404 - DNA, 37 bp, Artificial Sequence
  Affymetrix ID 2805739
ctacaatgcatctaataatgagctggttcgtaccaag

SEQ ID NO: 405 - DNA, 394 bp, Artificial Sequence
  Affymetrix ID 2806859
tagcattgatcttcatcaggcaggaagagtggactctcaggcttctataactcaggattcagactccataaaaaagcctgaagaaatcaaacaatgtaatgatgcacctgtttctgttcttcaggaagatattgttggaagtcttaaatctacaccagaaaaccatcctgagacacctaaaaaaaagtctgatcctgagctttcaaagagtgaaatgaaacaaagtgaaagtagattagcagaatctaaaccaaatgaaaaccgattggtggagacaaaatcaagtgaaaataagttagaaactaaagttgagacccaaacagaagaacttaaacagaatgagagcagaacaactgaatgcaaacaaaacgagagcaccatagttgagcctaaa

SEQ ID NO: 406 - DNA, 58 bp, Artificial Sequence
  Affymetrix ID 2806912
ccagcacttcttagatcccttttcaccgttggagcactatgtcggcattttgattttg

SEQ ID NO: 407 - DNA, 307 bp, Artificial Sequence
  Affymetrix ID 2807658
agcccaaaacaaatgtgaccacttattaatagaattgatttgttccatattttatcaaaataataataatgttatatccaactaaagagttataaccaagtgtcatatgtaaactttaattggatcctagtttgaaataatcagctataaagaaatttggagttaacttcagaaatttaaatatggcttgattagatgatattggggaattattacttttacaatggtattacggatttttaggagaatgtcattatctgtaggagatacttgaagtattttcacggtgataagtatgtctgtaaca

SEQ ID NO: 408 - DNA, 45 bp, Artificial Sequence
  Affymetrix ID 2809146
ctacactagatattttgtgattggcaagacatgtatttaaacaat

SEQ ID NO: 409 - DNA, 29 bp, Artificial Sequence
  Affymetrix ID 2810487
ccgtctttaaatcctgagtatgagagaga

SEQ ID NO: 410 - DNA, 29 bp, Artificial Sequence
  Affymetrix ID 2812307
aagcttaggacttgctgaatatacagatc

SEQ ID NO: 411 - DNA, 38 bp, Artificial Sequence
  Affymetrix ID 2812500
cagttgaactcatcattgtacgagaagtttcctcataa

SEQ ID NO: 412 - DNA, 82 bp, Artificial Sequence
  Affymetrix ID 2812573
gcaatgaaagaaaaggcggtcgatctcgttcccatactcgctcaaaatccaggtctagctcaaaatcccattctagaaggaa

SEQ ID NO: 413 - DNA, 107 bp, Artificial Sequence
  Affymetrix ID 2817404
ccagatagtgcctttatcaggtgaacgaagatactcaatgccaccattgtttcatacacattatgtaccagatatagtcagatgtgttccaccttttcgagaaattg

SEQ ID NO: 414 - DNA, 32 bp, Artificial Sequence
  Affymetrix ID 2817457
caaatttttgtatgatgtcaaatgcaataaaa

SEQ ID NO: 415 - DNA, 131 bp, Artificial Sequence
  Affymetrix ID 2818271
gcacgattccttttgcatgagaagcacctccaaaagggttggcctttagggctgtgcccaaatgagctttcttatactgtttatcatgccacttctggtctcgtcggtgactacggagcttcctagcagta

SEQ ID NO: 416 - DNA, 74 bp, Artificial Sequence
  Affymetrix ID 2818964
agagcattttaagaggtgcagcctctggaagtggatcaaactagaactcatatgccatactagatatgtttgtc

SEQ ID NO: 417 - DNA, 31 bp, Artificial Sequence
  Affymetrix ID 2822274
ttttagatggttacactgttagaacactatt

SEQ ID NO: 418 - DNA, 31 bp, Artificial Sequence
  Affymetrix ID 2823066
gtgaattggcagcattcatttggtaaattcg

SEQ ID NO: 419 - DNA, 51 bp, Artificial Sequence
  Affymetrix ID 2823854
cttcttcctgaatcacgatggaaaaaccttcttaaccttgatgttattaag

SEQ ID NO: 420 - DNA, 179 bp, Artificial Sequence
  Affymetrix ID 2824899
ctgtgcccacctacggcatgcctctatgtattggctactacagtgttttaaaaagtgtttcagatatttctctaattatgtacaacctaaaatgttggtgttttgtatggatcacaagtgcagcattccttaattccttctgctatatgtcacacagttgttatttggagaaccaagta

SEQ ID NO: 421 - DNA, 33 bp, Artificial Sequence
  Affymetrix ID 2828112
cttaacatactggaaattgcctaacttaatcat

SEQ ID NO: 422 - DNA, 105 bp, Artificial Sequence
  Affymetrix ID 2828637
tgtcaatagagacatacagcgcctaaagaacgacatagaagaacaagaaacactcttgggtacaataatgcctgaagaagaaagtgccaaagtatgcctgacaga

SEQ ID NO: 423 - DNA, 78 bp, Artificial Sequence
  Affymetrix ID 2828642
taatagctcaactaagtgaatgcgagaaacacaaagaaaagataaatgaagatatgagactcatgagacaagatattg

SEQ ID NO: 424 - DNA, 197 bp, Artificial Sequence
  Affymetrix ID 2829255
gctccaatgattgtttgctgctatccttatctacagtcatgctgagtaaagctatagctaaatggaagttaaattgtattcacgaggtgttaatgtttacatcttattatctgcagtctctcagagaaagcaaaagtaactacaaatagcgctatgccagaaactggtttcttgaccaacaatgtcgcttcagcatg

SEQ ID NO: 425 - DNA, 84 bp, Artificial Sequence
  Affymetrix ID 2829555
ttttatgtccatgaacttgagctactcgtgtgcaattatgtgtatgggaatggagtagtgttcatgatttgtatctacatcatc

SEQ ID NO: 426 - DNA, 188 bp, Artificial Sequence
  Affymetrix ID 2829679
taggtctgttgcaaattcatgattcttctgaggggggaaaccaaaagaacattagagtaaaaagaacgccactggaggatgtacaataaagcaccacaacacacgcttacaaacggggcttcctggcttcggtgacaggtaaaagacgctgttctccccactgctgtgcgtcaatcagggtttcatta

SEQ ID NO: 427 - DNA, 342 bp, Artificial Sequence
  Affymetrix ID 2831231
ttagtcagcagatgagtgccagtccagccttttctggtatgttattgttagaaatattgagttctaatgttacatctgaggaagtatgtaatttgagaattgtaacttctaagggattcactgcatcatagctatgcctgtatggagtctaacatatgaccaataccaacccataatccagctgaacaaagatactgtaacatgatttgagtggtgcttttccttgctttgttaaccatcacgagagtctgcagcacaacttttaacaaagctagaacagttttggcttcttaaacttcatatttgggtaggttaagctgccatacgtgttcagtgtgaata

SEQ ID NO: 428 - DNA, 38 bp, Artificial Sequence
  Affymetrix ID 2831557
acagccgcctgtatgtttcaataaatttgtccaaagct

SEQ ID NO: 429 - DNA, 68 bp, Artificial Sequence
  Affymetrix ID 2836687
atcttaaaggaatgaagtgtaatgcttgatgcacaggtgatatctactacatttaagcccataaagac

SEQ ID NO: 430 - DNA, 25 bp, Artificial Sequence
  Affymetrix ID 2840638
ggacaagaatccttcaagaaacagg

SEQ ID NO: 431 - DNA, 65 bp, Artificial Sequence
  Affymetrix ID 2843280
gcagagtcttagttactgatttcattttcaataaatgtaggtttgttacatgagtttcccaataa

SEQ ID NO: 432 - DNA, 51 bp, Artificial Sequence
  Affymetrix ID 2844248
tgagctttaaatcattagagcttaaattgtcagatcgttcattgcctttcc

SEQ ID NO: 433 - DNA, 31 bp, Artificial Sequence
  Affymetrix ID 2844250
tcctcacttttcaatttgtcatgttactaaa

SEQ ID NO: 434 - DNA, 213 bp, Artificial Sequence
  Affymetrix ID 2848258
cccacgattacacagctttgaacaagtgaaactatggttaccaaacagctatttttattatagcatctacagtgtctggaaaaggatgtaacaataaataactgtagatgcatcacgagacatccatttacttaatcacagaagtggatcttgctacatagttttctcaatgtcttcattcttcagattctccaggcttacgaatgtcatcaa

SEQ ID NO: 435 - DNA, 72 bp, Artificial Sequence
  Affymetrix ID 2848472
gcatgcatctgtgagctttagatcattaaatccaaaatgtttgcctaaatgagtttatcagttgttaacttc

SEQ ID NO: 436 - DNA, 62 bp, Artificial Sequence
  Affymetrix ID 2851973
gtggagtatttacttgctcagtaactgtgactgttgtctattgagacctagcctagttttcc

SEQ ID NO: 437 - DNA, 30 bp, Artificial Sequence
  Affymetrix ID 2854808
gcacctaaaaattgtataccgcagattcag

SEQ ID NO: 438 - DNA, 44 bp, Artificial Sequence
  Affymetrix ID 2854809
gccaaggctaaaagacgaaataccaccggaactggtcgaatgag

SEQ ID NO: 439 - DNA, 83 bp, Artificial Sequence
  Affymetrix ID 2855289
cctgacctcctttatggttaatactattaagcaagaatgcagtacagaattggatacagtacggatttgtccaaataaattca

SEQ ID NO: 440 - DNA, 58 bp, Artificial Sequence
  Affymetrix ID 2855291
tcttttgtatatctaagactcatcctgatttttactatcacacatgaataaagccttt

SEQ ID NO: 441 - DNA, 212 bp, Artificial Sequence
  Affymetrix ID 2855292
gtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataa

SEQ ID NO: 442 - DNA, 50 bp, Artificial Sequence
  Affymetrix ID 2857172
atggaagtaaatatgaaagagtggattttacgatatgctgaacaacaaaa

SEQ ID NO: 443 - DNA, 104 bp, Artificial Sequence
  Affymetrix ID 2862162
actactatgcttgaagaactgtaaaaatttgggaatcatgatgttgagaggacgatctaaaacatcactgtctaaaatctcagcagaatcttcacaaatcttct

SEQ ID NO: 444 - DNA, 60 bp, Artificial Sequence
  Affymetrix ID 2862377
gattcccaggcttcttcggcttaattagctgcaaagaatgaattgtgtataagcaaaaac

SEQ ID NO: 445 - DNA, 27 bp, Artificial Sequence
  Affymetrix ID 2865063
atattaatggtgaaaacactgtagtaa

SEQ ID NO: 446 - DNA, 415 bp, Artificial Sequence
  Affymetrix ID 2868141
attgatgattggtacgaaaaatgtcaaaatgtgttcccctaatacacgacaaaatagagtgacttctggacataaatctgccatttattaaaccattcactacaacaaataaataggtataaaagtggaattggaatttttatacttatttgttgtagtgaatggtttaataaaaatagaaatcactggtaatttccaccccaaactaaactatttcccttcttttaaaaaaatacacaaccaagattttaatgtaaaatattttgctttaattgtattttatgccttgattaatgaaacatggaaatattgattttcagttttggtcacctgaggaacctatctttgtttgcttttggaaaagcccattttctaaacagatacaatattgccacaacaatgtgcagaaaccttt

SEQ ID NO: 447 - DNA, 86 bp, Artificial Sequence
  Affymetrix ID 2877518
agtcttctggtggattaagcaaagatgatattgaaaatatggttaaaaatgcagagaaatatgctgaagaagaccggcgaaagaag

SEQ ID NO: 448 - DNA, 52 bp, Artificial Sequence
  Affymetrix ID 2878261
tattcttcagtccaaggaagcaattcacagtcagctgttagagaagcagaaa

SEQ ID NO: 449 - DNA, 219 bp, Artificial Sequence
  Affymetrix ID 2882121
tggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtg

SEQ ID NO: 450 - DNA, 87 bp, Artificial Sequence
  Affymetrix ID 2888344
gctagttctgcttgaccaagtagtgcaatcattattttttttaatattgttgctgatttcagagggatattcactaataaatgtatg

SEQ ID NO: 451 - DNA, 76 bp, Artificial Sequence
  Affymetrix ID 2893822
tgcagatgttcatgctcggtacattgaactacttacaagatctggagactattacaggttcttaagtgagatgctg

SEQ ID NO: 452 - DNA, 125 bp, Artificial Sequence
  Affymetrix ID 2893942
cttggaatacaagactcgtgatgcaaagctgaagttgtgtgtacaagactcttgacagttgtgcttctctaggaggttgggtttttttaaaaaaagaattatctgtgaaccatacgtgattaata

SEQ ID NO: 453 - DNA, 50 bp, Artificial Sequence
  Affymetrix ID 2896978
tttgtatgccaccaatttgtattatttgtctcaataaatacttagtcatc

SEQ ID NO: 454 - DNA, 29 bp, Artificial Sequence
  Affymetrix ID 2898531
tgtgtgattaataaacgtggaattaacag

SEQ ID NO: 455 - DNA, 30 bp, Artificial Sequence
  Affymetrix ID 2902010
tctctagtgtagctgccacattgatttttc

SEQ ID NO: 456 - DNA, 54 bp, Artificial Sequence
  Affymetrix ID 2902011
ttttacactaatatatggacctagcttgaggcaattttaatcccctgcactagg

SEQ ID NO: 457 - DNA, 116 bp, Artificial Sequence
  Affymetrix ID 2905137
ttttccatagctgggatataggctgcagctatagttgaacaagcagtctttaaaaactgctgtgaaacacaggccatcagggaaaacgaaatgctgcactattaaattagaggttt

SEQ ID NO: 458 - DNA, 108 bp, Artificial Sequence
  Affymetrix ID 2906968
ttaagctaaagtattgcctctattctgtaaagttctgtacatagttcccaagcttctgcagggggtgatttttgctcttgtcctgagaaataacagtgctgttttaaa

SEQ ID NO: 459 - DNA, 78 bp, Artificial Sequence
  Affymetrix ID 2908048
ctgggctctgtgtgaggcctggcccccactcccaaccttggctctagactgttactcttaagctttgagaaattttca

SEQ ID NO: 460 - DNA, 57 bp, Artificial Sequence
  Affymetrix ID 2909471
actggaaaaactgcgaaaagatttggaagaagagaagacaatgagaagtaatctaga

SEQ ID NO: 461 - DNA, 73 bp, Artificial Sequence
  Affymetrix ID 2910489
agcaatttagaaaatcgaccttgcagagcagcaagaggctctctccagaaaacatcggcagataccaaaggaa

SEQ ID NO: 462 - DNA, 227 bp, Artificial Sequence
  Affymetrix ID 2914002
gttacatgacctttcagttgactgaaattcttaggctacaagagttaaataaataatttgtttattgcattataaacagttccttataactagtaacattttaagatttggcatatattttagatactgttcttcattttttaaaaatgtaatatctagactgctcgtcatttagaagacacgtcctgaaccttgctatatttttctgtttacatagatgggctatc

SEQ ID NO: 463 - DNA, 129 bp, Artificial Sequence
  Affymetrix ID 2914107
gaatccttaatatgtgaatccagagataagtttatacgggaattatttgaatcatccacaaataacaacaaagatactaaacaaaaagcaggaaaacttagcttcatcagcgtgggaaacaagtttaag

SEQ ID NO: 464 - DNA, 39 bp, Artificial Sequence
  Affymetrix ID 2916383
ttctataatgttaatggggaaattcagcaataaacttta

SEQ ID NO: 465 - DNA, 54 bp, Artificial Sequence
  Affymetrix ID 2916976
ttgcaagtagaatatggaaaatgtcaactacaaatgaaagagctgatgaaaaaa

SEQ ID NO: 466 - DNA, 308 bp, Artificial Sequence
  Affymetrix ID 2918543
ggtacactgtgttagctcgcctacataaaaaatattgcacattttattttgtctccagatgtgatcgatgtctaaactatatagaatttcaaactattttaaagttattctgtgaaatgtcatctttataaatatcagaactaaatgcactgtaaacagccattttggtaaacatgcatttattcacagttgatttatttcctttagataaatgtgtaactgctcaaaattatataataatctatgtagaggtacaagagcacactatacaaatctatttctatcttcatgttgaggaaacactgatc

SEQ ID NO: 467 - DNA, 296 bp, Artificial Sequence
  Affymetrix ID 2918547
gcgactggcacgattggttcgtctatcccttgagcgacttctacttctacgtctctctcgggaaggactccgatttcgtcgtctttctctttcaatgctatttctattagatctccttctatctctaatctttaccctagccctattgctctctattttaattctgcgagaatagcttctactcctgctacgtctagctttgattgttggagatctactcctactgtgtctctttttccttttaggagaagaagaccgagaagaagtacgactactacctgagctagaactgtatg

SEQ ID NO: 468 - DNA, 92 bp, Artificial Sequence
  Affymetrix ID 2922232
cttgcatgccatcaggagtaaattttattgtggttcttaatctgaagttttcaagctctgaaattcataatccgcagtgtcagattacgtag

SEQ ID NO: 469 - DNA, 78 bp, Artificial Sequence
  Affymetrix ID 2922235
tggcatttgctgtggttacaaaatacttcctctgggttataataatcatttgatccaattcctattgcttgtaaaata

SEQ ID NO: 470 - DNA, 30 bp, Artificial Sequence
  Affymetrix ID 2926222
gatgtcattgaagagtatttcaaatgcaag

SEQ ID NO: 471 - DNA, 28 bp, Artificial Sequence
  Affymetrix ID 2929946
atggacattcagtttcagcaggaattag

SEQ ID NO: 472 - DNA, 45 bp, Artificial Sequence
  Affymetrix ID 2930652
gttgatggttttgtgatttagctgggtaaactatctttgtaacag

SEQ ID NO: 473 - DNA, 85 bp, Artificial Sequence
  Affymetrix ID 2932399
tctcacgtttcagaatctgtgaacaattccatttttcatcagatagcagaacaactacaacagcaaaacctagaacatctcagac

SEQ ID NO: 474 - DNA, 28 bp, Artificial Sequence
  Affymetrix ID 2932428
tagatgccaataaaacttatttgtttga

SEQ ID NO: 475 - DNA, 38 bp, Artificial Sequence
  Affymetrix ID 2933660
acctaaagtgatccagacttctgcggctaatttttcac

SEQ ID NO: 476 - DNA, 52 bp, Artificial Sequence
  Affymetrix ID 2936963
tttttcacaaggtcaagatgtatccaataaagtgaaagcttctcgtaaatta

SEQ ID NO: 477 - DNA, 245 bp, Artificial Sequence
  Affymetrix ID 2940827
aggggatgtggatacttggcccaaagtaactggtggtaggaatcttagaaacaagaccacttatactgtctgtctgaggcagaagataacagcagcatctcgaccagcctctgccttaaaggaaatctttattaatcacgtatggttcacagataattctttttttaaaaaaacccaacctcctagagaagcacaactgtcaagagtcttgtacacacaacttcagctttgcatcacgagtcttg

SEQ ID NO: 478 - DNA, 82 bp, Artificial Sequence
  Affymetrix ID 2942512
gatgcattcagtctactagcatattcagatccctggaacagcccagttggaaatcagcttgacccgattcagagagaacctg

SEQ ID NO: 479 - DNA, 213 bp, Artificial Sequence
  Affymetrix ID 2944070
catgctgtttatgacacaatgacacatttgttgcaccaattaagtgttgaaaaatatctttgcatcatagaacagaaatatataaaaatatatgttgaatgttaacaggtattttcacaggtttgtttcttgatagttactcagacactagggaaaggtaaatacaagtgaacaaaataagcaactaaatgagacctaataattggccttcga

SEQ ID NO: 480 - DNA, 217 bp, Artificial Sequence
  Affymetrix ID 2944963
tcaacaatctgacaggcagtgaacttgacatgattagctggcatgattttttcttttttttcccccaaacattgtttttgtggccttgaattttaagacaaatattctacacggcatattgcacaggatggatggcaaaaaaaagtttaaaaacaaaaacccttaacggaactgccttaaaaaggcagacgtcctagtgcctgtcatgttatattaa

SEQ ID NO: 481 - DNA, 180 bp, Artificial Sequence
  Affymetrix ID 2946479
gtaaatgaagagctgtaccatattcatgtattggaagacaacattgtaaagatgacatggtttaccagattaatctataaattcaatacaaatccaatcaaaatttcaatgctcttgggtttgtttgatttataaattgttggtctaattctagaagtaatatggaggaacagttggcta

SEQ ID NO: 482 - DNA, 40 bp, Artificial Sequence
  Affymetrix ID 2947592
aaatgtctattcttgacggagagtctaaagcagttcacag

SEQ ID NO: 483 - DNA, 31 bp, Artificial Sequence
  Affymetrix ID 2948592
gtcaatgcaataggtctcatcagtattctct

SEQ ID NO: 484 - DNA, 33 bp, Artificial Sequence
  Affymetrix ID 2948596
tcaactttgctacccaaataaattgctgtaagt

SEQ ID NO: 485 - DNA, 392 bp, Artificial Sequence
  Affymetrix ID 2949053
acctttcctgtaaacctgtagatggtttaattcttagtcaagagaccagtctcatctaaaactatcctgttgtggtctgacggcaagtaactcatcttgagtaatttttgtttctccttaagtggcattttgactgtccattgcagcattctgatcttaaaagacatccactttgctaatgcacacgagattctcttagttgaagtaggagaatcaaatggagcagttgtcctccccccaccccatgttcttagaagcacctctgatggagttattctgaccttgagtcactgcctcccatcatttcccagatgtttggtccttgctctccctttgagaatcatctcccattttctttcctctcccacctctatttgaggtaatggcatctg

SEQ ID NO: 486 - DNA, 31 bp, Artificial Sequence
  Affymetrix ID 2949061
attttggatgaatgtgataagatgcttgaac

SEQ ID NO: 487 - DNA, 80 bp, Artificial Sequence
  Affymetrix ID 2951060
gggcttgggcacaaatccccaggcaggctttggagttgtttccatggtgatggggccagatgtatagtattcagtatata

SEQ ID NO: 488 - DNA, 52 bp, Artificial Sequence
  Affymetrix ID 2956077
agaatccaactcttctgcctctgttagaccaaaggtactgagtagcatccag

SEQ ID NO: 489 - DNA, 113 bp, Artificial Sequence
  Affymetrix ID 2958402
aaggtatctcggtggtagcacagcaatttcatgaaaccttagaaccactgaacgagtggcttacaaccatagaaaagaggctggtgaattgtgaacccataggaacccaagca

SEQ ID NO: 490 - DNA, 83 bp, Artificial Sequence
  Affymetrix ID 2961302
tcccagcaatcgcttggttcagtttcattcagctctctatggaccagtaatctgataaataaccgagctcttctttggggatc

SEQ ID NO: 491 - DNA, 79 bp, Artificial Sequence
  Affymetrix ID 2966293
ggcatacataatcttgctgctatgcttcgaagctgtagtctgaatcaacctaagttttaaacagaaggtgaacctctga

SEQ ID NO: 492 - DNA, 170 bp, Artificial Sequence
  Affymetrix ID 2966378
actgtactgtctgtggaacttagcaaattaatcattttcttcagacttgaaggagagtgataaataaaatttggagtcataggatattgatgcacaatttaaggattaaacatttttaatcaattgtggatgatggcttattaaatgttgacttcctagtataaaactgc

SEQ ID NO: 493 - DNA, 218 bp, Artificial Sequence
  Affymetrix ID 2967164
ttatctgtgagctctgacacatctgaaaaagtaatctgatgtgcaaattataatttaaatatgttaatttttttgcttcttaaatttgcttttcatcattaaaatgtcaagttcaagtgatatgtgcctaatatcacttggatgttggtgggtttttgaatttttgggtggttaatcagttttattttgaaaagacgtacttgaatagttacagcata

SEQ ID NO: 494 - DNA, 34 bp, Artificial Sequence
  Affymetrix ID 2967323
ctcatggagaagacttctttgcaacaataataaa

SEQ ID NO: 495 - DNA, 34 bp, Artificial Sequence
  Affymetrix ID 2968257
gggccgcttcaccacttacgaaatcagggtcaag

SEQ ID NO: 496 - DNA, 58 bp, Artificial Sequence
  Affymetrix ID 2968653
actttgtattctggaagcgtgaattgcttttgaagtctgtcagtattactggtatttt

SEQ ID NO: 497 - DNA, 54 bp, Artificial Sequence
  Affymetrix ID 2974195
ggggaattggcattgcgaaagacttgaaaactaatgagtaaagtctgctgaatg

SEQ ID NO: 498 - DNA, 36 bp, Artificial Sequence
  Affymetrix ID 2974332
tctactttgatatgactgtttttcggacagtttatt

SEQ ID NO: 499 - DNA, 365 bp, Artificial Sequence
  Affymetrix ID 2975686
tgcggcattgatctaacctttttcccccccatttcagttgcagtttttgtagaatggctttttctttttcctcttaagagttctattcttcaggtagataatttttcaaatgtgaattatcttttgtgtctatattgatagctcttaaaggagtgaaaatctaaaatagtaaatttcaatgttaagtgtctgctttatgggcatatataaaagtagacacatttcatttgttaatttagttgtgtgtgtgtgttaaaaggagctaatgcttattctgttaatgtaaacttttgaagatcttaagtgtattgctctttcatcttaaacactttcgaggatttgcagtgcgtctagcacctagatta

SEQ ID NO: 500 - DNA, 37 bp, Artificial Sequence
  Affymetrix ID 2975709
ctaaacgagcggttcacttcgtatcagaaagccactg

SEQ ID NO: 501 - DNA, 53 bp, Artificial Sequence
  Affymetrix ID 2976368
tatatatgatgcatcggataggtcattatgattttttaccatttcgacttaca

SEQ ID NO: 502 - DNA, 36 bp, Artificial Sequence
  Affymetrix ID 2977521
tccagtgcactttgccattaaagtctcttcacattg

SEQ ID NO: 503 - DNA, 123 bp, Artificial Sequence
  Affymetrix ID 2981918
ttcgaaatgagctcaaattgattttttaatttctatgaaggatccatctttgtatatttacatgcttagaggggtgaaaattattttggaaattgagtctgaagcactctcgcacacacagtg

SEQ ID NO: 504 - DNA, 72 bp, Artificial Sequence
  Affymetrix ID 2982323
tttgaatttctattgagcgcctttaaaaaagtaataccaaaccataaagttaaaatctatgtatattgagtc

SEQ ID NO: 505 - DNA, 31 bp, Artificial Sequence
  Affymetrix ID 2982385
atttgagcaatggtaaacctcgagacaacaa

SEQ ID NO: 506 - DNA, 157 bp, Artificial Sequence
  Affymetrix ID 2984576
agcacttgaatgttggtactctatgtttggtgaagtttgcttttccccataaaacactccaggaacaactgacgtgacagttgaagaccgttttgtactaagtctcattttgtatactggtaaaaactacatgcttgattaaaccattaaatgcttg

SEQ ID NO: 507 - DNA, 206 bp, Artificial Sequence
  Affymetrix ID 2985813
tgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctcta

SEQ ID NO: 508 - DNA, 34 bp, Artificial Sequence
  Affymetrix ID 2985965
gagagaatttcttttgggcagtgaaaataaagca

SEQ ID NO: 509 - DNA, 56 bp, Artificial Sequence
  Affymetrix ID 2988895
ggtgctctttcatgcctattatcagtaaggggacttgtattagagtcagagtcttt

SEQ ID NO: 510 - DNA, 32 bp, Artificial Sequence
  Affymetrix ID 2988896
aggccagttgtcaagtgtcaataaaagcatca

SEQ ID NO: 511 - DNA, 45 bp, Artificial Sequence
  Affymetrix ID 2989720
cctgctgtacctggaaatcaagtttgagcttcgagaataaaggtc

SEQ ID NO: 512 - DNA, 151 bp, Artificial Sequence
  Affymetrix ID 2991248
ggaaagatggatcaatacttccacctcagttggctttgtttgcgatagctactccacttcagccaccatccatacttgaaatccggaccaaaaattttatctttagaaccaaacacaaactagacttcacacctattggttgtgatgccaa

SEQ ID NO: 513 - DNA, 490 bp, Artificial Sequence
  Affymetrix ID 2991263
aaactgtcactgttggacgtcagcaagttcacatggaggcattgatgcatgctattcacaattattccaaaccaaattttaatttttgcttttagaaaagggagtttaaaaatggtatcaaaattacatatactacagtcaagatagaaagggtgctgccacggagtggtgaggtaccgtctacatttcacattattctgggcaccacaaaatatacaaaactttatcagggaaactaagattcttttaaattagaaaatattctctatttgaattatttctgtcacagtaaaaataaaatactttgagttttgagctactggattcttattagttccccaaatacaaagttagagaactaaactagtttttcctatcatgttaacctctgcttttatctcagatgttaaaataaatggtttggtgctttttataaaaagataatctcagtgctttcctccttcactgtttcatctaagtgcctcacatt

SEQ ID NO: 514 - DNA, 709 bp, Artificial Sequence
  Affymetrix ID 2991264
gttgctatgtgccttatgttgaaaaaatttaaaagtaaaatgtctttccaaattatttcttaattattataaaaatattaagacaatagcacttaaattcctcaacagtgttttcagaagaaataaatataccactctttacctttattgatatctccatgatgatagttgaatgttgcaatgtgaaaaatctgctgttaactgcaaccttgtttattaaattgcaagaagctttatttctagctttttaattaagcaaagcacccatttcaatgtgtataaattgtctttaaaaactgttttagacctataatccttgataatatattgtgttgactttataaatttcgcttcttagaacagtggaaactatgtgtttttctcatatttgaggagtgttaagattgcagatagcaaggtttggtgcaaagtattgtaatgagtgaattgaatggtgcattgtatagatataatgaacaaaattatttgtaagatatttgcagtttttcattttaaaaagtccataccttatatatgcacttaatttgttggggctttacatactttatcaatgtgtctttctaagaaatcaagtaatgaatccaactgcttaaagttggtattaataaaaagacaaccacatagttcgtttaccttcaaactttaggtttttttaatgatatactgatcttcattaccaataggcaaa

SEQ ID NO: 515 - DNA, 32 bp, Artificial Sequence
  Affymetrix ID 2993622
ttgaccacttcttcattagttacctgcagcaa

SEQ ID NO: 516 - DNA, 28 bp, Artificial Sequence
  Affymetrix ID 2993644
taatagctcttcaagtctgcaataaaaa

SEQ ID NO: 517 - DNA, 680 bp, Artificial Sequence
  Affymetrix ID 2993659
gttctcatgagcctaaggaccattctagatttattacgtgttttttgtgtgtgtgtgtgtgtgtgtgtgtgtgtgtatccataaaatgcatatgtaaatttttttttgtttttaagcattcacccaaacaaaaaaatcacaggtaaacccatgtttctgagatgccattattccaagcaaaataagagataatcccttcaagttaaattgaaaattttcctgaaaccatacatttcaagtgaaataagtaattctagataggacaatttaaattggataattttaaagtgtctataattgcagtggtttatttgcaaaattcctaaaaggaaaaattttatcactgccatcacagcaggtttcctcatccagatgaggaaactagacaaatgctagtgtgttttaactagctaaacaaaactaagttaaatgaacatttaaaagtttccctagcgggccattccttagcaaaatgttggaatccctgttgctacattgactaaaaggtcatgatgaatggaatatgtaagacttggctcatagaaacctaatcagatggttagaggtgttggcagtttaggacctgctgtcataaatgtgtgaacaaccttttgtaacctaacctattgacctgcatgttttttctttaccccaattcattacatggaggctcaatcttg

SEQ ID NO: 518 - DNA, 28 bp, Artificial Sequence
  Affymetrix ID 2994371
gtaatcatctataagccatgggaagcta

SEQ ID NO: 519 - DNA, 30 bp, Artificial Sequence
  Affymetrix ID 2995598
gcatatatgtctctttggagttggaatttc

SEQ ID NO: 520 - DNA, 25 bp, Artificial Sequence
  Affymetrix ID 2997143
tacagaagcagaaaacttgcagctg

SEQ ID NO: 521 - DNA, 121 bp, Artificial Sequence
  Affymetrix ID 3002667
gttgcacttggcctagcattccaacctcacctgcctcagcttgttcaacctgaaaacctaccaagtgaaagcaagagccacgtgaagacgccttagttatatgcacccacccagacacttg

SEQ ID NO: 522 - DNA, 29 bp, Artificial Sequence
  Affymetrix ID 3003180
tgtgaatgattttctcaaagaacatagaa

SEQ ID NO: 523 - DNA, 31 bp, Artificial Sequence
  Affymetrix ID 3004652
tttttggattatgcgtgaacttagtttttta

SEQ ID NO: 524 - DNA, 31 bp, Artificial Sequence
  Affymetrix ID 3011652
gtctgcccagaaagttcagaaggctaaataa

SEQ ID NO: 525 - DNA, 31 bp, Artificial Sequence
  Affymetrix ID 3011846
tttatcaccaacatttcaagtttgtatttgt

SEQ ID NO: 526 - DNA, 96 bp, Artificial Sequence
  Affymetrix ID 3012405
cagcactcatagtagcacagctgcagacttactacaagccaaacaacagatcctcactcatcaacagcagcttgaagaacaagaccacttattaga

SEQ ID NO: 527 - DNA, 122 bp, Artificial Sequence
  Affymetrix ID 3012428
ggatgcttctagacaactaatgttgaatgaagaacagttggaagatatgagacaggaacttgtacgacaataccaagaacatcaacaggcaacggaattgttaaggcaagcacatatgcggc

SEQ ID NO: 528 - DNA, 35 bp, Artificial Sequence
  Affymetrix ID 3012438
tccctcttattctggaagtgatatgccaagaaatg

SEQ ID NO: 529 - DNA, 57 bp, Artificial Sequence
  Affymetrix ID 3012452
aaacactttggagctgtagaagctaaaccagaattgtccctagaagtacaattgcag

SEQ ID NO: 530 - DNA, 32 bp, Artificial Sequence
  Affymetrix ID 3012453
gctgaacgagatgccatagacagaaaggaaaa

SEQ ID NO: 531 - DNA, 66 bp, Artificial Sequence
  Affymetrix ID 3013190
gaagcaaagaactgccttgtagataaacatattgcatttattggagattccagaattcgtcaattg

SEQ ID NO: 532 - DNA, 198 bp, Artificial Sequence
  Affymetrix ID 3013468
tgcagccaatgcttcaccatcaagaatgatgtttgctgtagttttgtgggaattttccttatctttctagtttgcttggaactttaggaatgagtattgaattttattaaatgctttttggtactcactgagatgatataggtcttttctcttttaaatcagttaataaattacctatcagtctatctaaaatggtga

SEQ ID NO: 533 - DNA, 224 bp, Artificial Sequence
  Affymetrix ID 3013685
atgcgcctctgaccattatgcaaggcaacatgaattaaatacttatgtcagaacatatttgtcttacattattcaagataaagtggattttaaaagcaagtgggtaccaatgatggggagatgaggagaatagggcaggcagcaacagggcaacatgcatttttcaagagtgtttattaaaataggcagtaatcagtacatgtacatcatatgagcagtttttc

SEQ ID NO: 534 - DNA, 45 bp, Artificial Sequence
  Affymetrix ID 3014422
ctggattttgtgaaacagatttacaaggagcttccaaaagtagtg

SEQ ID NO: 535 - DNA, 95 bp, Artificial Sequence
  Affymetrix ID 3015985
ccgttcctcagtcctgtatcatccatacttgtactaccttgtcctatgaagctctgagaattttttgtacgatcagccttactgctaataaaagc

SEQ ID NO: 536 - DNA, 30 bp, Artificial Sequence
  Affymetrix ID 3017629
aagtccactactattaaatgacagctgttc

SEQ ID NO: 537 - DNA, 32 bp, Artificial Sequence
  Affymetrix ID 3017826
ttgtggataatcaaacatagccaataaatttt

SEQ ID NO: 538 - DNA, 320 bp, Artificial Sequence
  Affymetrix ID 3021760
ctacatcggtgtagtgatgtctacagaactgtgcaatgtatcagcttcaacattcatatgtagcattgtattcaaaataaaggggctaaagaaactaaggtctgcatcacttaaaaaagaatgtagtgctatactagatttttttttaagaaaatttaggtacagaaaaagtagggtatgaaaatagtgttttccttctggcattataagtaaattacattaaggtttttgtcagtctcacatatagtatttaaactcccttgttgatggaatgaaaaatattcacaaatcaagtgagcatcctggtgtaaacttgcaca

SEQ ID NO: 539 - DNA, 46 bp, Artificial Sequence
  Affymetrix ID 3022682
ggccagcccattctgtatttaaagcttttgaggcccaataaaatag

SEQ ID NO: 540 - DNA, 46 bp, Artificial Sequence
  Affymetrix ID 3027555
ggtgagaatttcaaggattatcgacttcatattgcacattaaagtt

SEQ ID NO: 541 - DNA, 65 bp, Artificial Sequence
  Affymetrix ID 3027926
gacaacatggcacagaatatcagtattccggccaggcctcagagacgtggcatatcaatatgtga

SEQ ID NO: 542 - DNA, 57 bp, Artificial Sequence
  Affymetrix ID 3027936
gtacagtctcatttccaagtcatgtataatctttatggcttccaaggacaagaatta

SEQ ID NO: 543 - DNA, 75 bp, Artificial Sequence
  Affymetrix ID 3037391
tgcatcttgccttatctttttttattactgtgtacaaagatttttttacacaaagaaacttaatgctgtattaat

SEQ ID NO: 544 - DNA, 37 bp, Artificial Sequence
  Affymetrix ID 3037406
tgcactggtcttcacaactcgttacctggatcttttt

SEQ ID NO: 545 - DNA, 44 bp, Artificial Sequence
  Affymetrix ID 3037950
tgatggattttcatacgattgtaaatgagctatattaaagtcta

SEQ ID NO: 546 - DNA, 338 bp, Artificial Sequence
  Affymetrix ID 3039980
ggggaactaataagaatccagtagctcaaaactcaaagtattttatttttactgtgacagaaataattcaaatagagaatattttctaatttaaaagaatcttagtttccctgataaagttttgtatattttgtggtgcccagaataatgtgaaatgtagacggtacctcaccactccgtggcagcaccctttctatcttgactgtagtatatgtaattttgataccatttttaaactcccttttctaaaagcaaaaattaaaatttggtttggaataattgtgaatagcatgcatcaatgcctccatgtgaacttgctgacgtccaacagtgacagt

SEQ ID NO: 547 - DNA, 133 bp, Artificial Sequence
  Affymetrix ID 3039983
ggtagggtgattaatttgcctattggtaatgaagatcagtatatcattaaaaaaacctaaagtttgaaggtaaacgaactatgtggttgtctttttattaataccaactttaagcagttggattcattacttg

SEQ ID NO: 548 - DNA, 74 bp, Artificial Sequence
  Affymetrix ID 3040116
tggcaaccttattttactacacgcattgtagatgactttggcacacacttacgagtattcagaaaggctcaaca

SEQ ID NO: 549 - DNA, 35 bp, Artificial Sequence
  Affymetrix ID 3041262
aaggctcttcacagacgcatctgtagcagagtgga

SEQ ID NO: 550 - DNA, 200 bp, Artificial Sequence
  Affymetrix ID 3042004
cagatctataaggtcacatgcttttaaaatagtagcaagttaaacttcactcttgaattctttacaatctaagtcaaactaagttataatttaggattgtctttaaacagccattcagaaacaaaactgtagaactgtgtatttgattgggaatggtgcttttgccaacttaaaaggattaaagtaacggagatatacac

SEQ ID NO: 551 - DNA, 136 bp, Artificial Sequence
  Affymetrix ID 3042424
tttagttgaggttcttgatataaaacaattacaaaatgagtgttgtttgtaaaacagtaacatcaaattggctagagagataaatgtatcatgttttaaattaggttttgtgagtagacagattacaattctattt

SEQ ID NO: 552 - DNA, 28 bp, Artificial Sequence
  Affymetrix ID 3042426
gtcttgctgcaggtaactaatgaagaag

SEQ ID NO: 553 - DNA, 60 bp, Artificial Sequence
  Affymetrix ID 3042429
accatggacaagtatattctgctgccacaaagactgtaaagtgcttcatttcaacagctg

SEQ ID NO: 554 - DNA, 230 bp, Artificial Sequence
  Affymetrix ID 3042430
gataaactaatactcttaaatggtctttcagtatagtggttcttacgtagtttaacatagctataaattgagtttaacaatttataaactcaagagaataatttttataaaccctgttttccaatctgtcatttacttaaattattttggttgtttttccctttttttccttcttttcccaccccctccccctccatgtgaagatttgggtgcttaacatatcatttttt

SEQ ID NO: 555 - DNA, 163 bp, Artificial Sequence
  Affymetrix ID 3042437
gcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatg

SEQ ID NO: 556 - DNA, 71 bp, Artificial Sequence
  Affymetrix ID 3042453
tgcagaaataccataccatcaatggtcataatgcagaagtaagaaaggctttgtctagacaagaaatgcag

SEQ ID NO: 557 - DNA, 31 bp, Artificial Sequence
  Affymetrix ID 3048780
cagaattttccagtgaaataaaacatgttgt

SEQ ID NO: 558 - DNA, 356 bp, Artificial Sequence
  Affymetrix ID 3048870
cttcaaacgtctgcattcatgtataatagccattttatgcacagatgaggtgattcccacactttttttcgcagatgtttcattatctggatgagtgtattacataataaatttgaataatggtagtcttccaaatgacaaaaagcaagctagttgatttgtcaattttgagtttaaatgttttgactgcttatcagcaaaaaaaatcaaagaatagcatactaatattaatggaaatgcagagtatatttaattggcatgattttttcatggatgtgtgcttcatttgatctattgtatgtagctcgtgatcacattttctgttggttaacattgttcctagcttatatgatgga

SEQ ID NO: 559 - DNA, 30 bp, Artificial Sequence
  Affymetrix ID 3053384
gatacaggcatatgaataggtattaatcac

SEQ ID NO: 560 - DNA, 25 bp, Artificial Sequence
  Affymetrix ID 3056395
aggtcatctgaaagcatcgggtttg

SEQ ID NO: 561 - DNA, 479 bp, Artificial Sequence
  Affymetrix ID 3060052
tttgccttaacccttctgtagtacaggaatgattcttagatttgtttccttttgttatagaagcaaatattgtttttttaaaatagcctgagatgagaggttatattgtaccccaccagctaacacactaagtggatgacaaactattctctcggtaatttatatagcaaaacatctaataaatggtcatggtatcaaggataggtaacattacttccttcctcatttagttttacttcaaagtgctaactttgtaaactaatgacagtggtttctgagggggtgaaaactgcttagattctggtgggttttgttactatgcttatccatattccctttaaaagtaacaaaaaacctgtagtgaattttaacttttaatgctttcctgtttaccctgcctaaatcagctcccactgccagcaatgggttaaccaaatgtaccaccacaagaccacctcctaggtctctatacttgtatg

SEQ ID NO: 562 - DNA, 315 bp, Artificial Sequence
  Affymetrix ID 3061054
tactttcaacaaggcatatagtctcttgtaatatatatgtatactattacattgtacagaatctgtacttatgtttggtgtgtaatataccaattagttttatataatatcctcataagctattctaatttttgagagaaacacaaaaagttatagattgatagtctgtataatattagacaaaatagtaagctttataaaaagttatttttggtaaatggtaaggaatttggggtggaatgtgatgagattaaaatgtagctttggtataacttcatgtgtatttcaaaatatactgaacgtcaacatgatttg

SEQ ID NO: 563 - DNA, 77 bp, Artificial Sequence
  Affymetrix ID 3061144
ctcacttatttaactggcaactatccatttaggttaggcaaaggcacggtaacatgttgcgcaggatgttttactga

SEQ ID NO: 564 - DNA, 116 bp, Artificial Sequence
  Affymetrix ID 3061758
acagtgtaagtgaacctgctgttgccctcagcaacaagttcaacatcattagagccctgtagaatgacagcctttttcaggttgccagtctcctcatccatgtatgcaatgctgtt

SEQ ID NO: 565 - DNA, 483 bp, Artificial Sequence
  Affymetrix ID 3062023
caggcctgacctaaagctgtagtaatttaaaaagtgttatattggcacaggaatagacatgagacattaaaataacaaaacagagagtccctaaatagaccaatatgtaaatgtcagagatctagcacatgataaaggtgatattttgaatcagtgggagaaggatggactgtatgccaggcactgttccaggcactgggatctagcagtgaacaaatttccacagtcagtatgtagatgtcacttttctccaccctcagctctccctggaatcacctagtccaccaaccttctgattactctctgaagagaataaatccctgatcttcttttcagttattttcaggctttcagtattccacctccccgttattattttttctgttatcttttctctgttttttgagtttttgtggctttatgccactactttaaccacaatctctatttatccttcatgtacagttcccatgttagtccata

SEQ ID NO: 566 - DNA, 121 bp, Artificial Sequence
  Affymetrix ID 3064354
aaggctgatcgtacaaaaaattctcagagcttcataggacaaggtagtacaagtatggatgatacaggactgaggaacgggggacggctcaaaagaaatcaacatcgtctggggcatccag

SEQ ID NO: 567 - DNA, 35 bp, Artificial Sequence
  Affymetrix ID 3075645
tttgagacatcactagcctataataaatgggttaa

SEQ ID NO: 568 - DNA, 40 bp, Artificial Sequence
  Affymetrix ID 3076355
tctatgtacttgatgtgaatatgcgaatactgctataata

SEQ ID NO: 569 - DNA, 29 bp, Artificial Sequence
  Affymetrix ID 3076393
tcaccagcgttgtagtacagaagttccac

SEQ ID NO: 570 - DNA, 48 bp, Artificial Sequence
  Affymetrix ID 3076490
gccgtgtaatgtgaggtttacctgattctctaatgaaataaataccta

SEQ ID NO: 571 - DNA, 45 bp, Artificial Sequence
  Affymetrix ID 3081625
tgggatggtttcttggctttaagtccactgaataaaaactatgaa

SEQ ID NO: 572 - DNA, 165 bp, Artificial Sequence
  Affymetrix ID 3087828
tcattaactagggaggtttcccagagcaggaaaccatcagcttcagaacgtttacctgatgagaaagtcgaactttttagcaaaatgagagtgctacaggaaaagaaacaaaaaatggacaaattgcttggagaacttcatacacttcgagatcagcatcttaac

SEQ ID NO: 573 - DNA, 279 bp, Artificial Sequence
  Affymetrix ID 3087930
taccctgttgatacaatagcacaccacccataatgaaatacaagaaatcacacaggacagttcatattgttcagttctatttatacaaagttcacgaagaggcaaaactaatctatagtgacatcagaatagtgtttactggaggaggcagtattttctgcaaaggggcatgatggagcatcgtggagtgctgggctcttccgtatcttgatctgagtcatggctacatgcatgcatgcatttctgaagcttcactgagctgtatatctaagacctgtg

SEQ ID NO: 574 - DNA, 30 bp, Artificial Sequence
  Affymetrix ID 3088570
aatgtgacatctggatataaaatgaaaata

SEQ ID NO: 575 - DNA, 39 bp, Artificial Sequence
  Affymetrix ID 3092505
aaactatttcttgacgacctttgagagatttcaataaaa

SEQ ID NO: 576 - DNA, 28 bp, Artificial Sequence
  Affymetrix ID 3094309
ccatctgtgccaattacaaagcaattaa

SEQ ID NO: 577 - DNA, 325 bp, Artificial Sequence
  Affymetrix ID 3096162
ctgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacac

SEQ ID NO: 578 - DNA, 68 bp, Artificial Sequence
  Affymetrix ID 3096409
gctgcatgaggccaataatgaactacagaagaagagagccattattgaagatctcgagccaagattta

SEQ ID NO: 579 - DNA, 92 bp, Artificial Sequence
  Affymetrix ID 3096979
ccacgtgcagtttgcatcggatgcaagacagattatggagagactgatagatccaaggacaaaatcaacagagaccattttgcatacacctc

SEQ ID NO: 580 - DNA, 97 bp, Artificial Sequence
  Affymetrix ID 3099780
tactttgggatggtcttagaatatttttctgataacttgttccttttcctgactcctccttgcaaacaaaatgatagttgacactttatcctgattt

SEQ ID NO: 581 - DNA, 76 bp, Artificial Sequence
  Affymetrix ID 3100230
tttgatttctaggtcaattgatgtgattatttttgttaaatgttgtcttgtgcccttaactacgaactgaattgta

SEQ ID NO: 582 - DNA, 28 bp, Artificial Sequence
  Affymetrix ID 3107638
gtgcgattaaattatgcactggatggat

SEQ ID NO: 583 - DNA, 59 bp, Artificial Sequence
  Affymetrix ID 3108472
tttccttgaagaccataagcactagtgatccagccgaagtactcgtcaaaaatagccag

SEQ ID NO: 584 - DNA, 30 bp, Artificial Sequence
  Affymetrix ID 3108501
tttgccctgaacatgttggttgcaatcact

SEQ ID NO: 585 - DNA, 96 bp, Artificial Sequence
  Affymetrix ID 3108938
tatattcctactcgacatacaagtgttactctcctcaaatgtacctgcacaatttccatggctgaattcaacttgctggaccatttactacctgtc

SEQ ID NO: 586 - DNA, 173 bp, Artificial Sequence
  Affymetrix ID 3111090
ggccattaaggaagatcagattgcagataactttcaaggaatatcaggtcctaaagaagacagcacaagtataaaaggtaattcagaccaggattcttttcttcatgagaattcgttacaccaagaagagagtcaaaaagaaaatatgccttgtggggaaacagcagaattta

SEQ ID NO: 587 - DNA, 31 bp, Artificial Sequence
  Affymetrix ID 3111116
ttctactaaaataaggtcatagtggcatata

SEQ ID NO: 588 - DNA, 161 bp, Artificial Sequence
  Affymetrix ID 3112517
actgttatagccaaaatcggcaatgacactaaagaaatcctctgtgcttttcaatatgcaaatatatttcttccaagagttgccctggtgtgacttcaagagttcatgttaacttcttttctggaaacttccttttcttagttgttgtattcttgaagagc

SEQ ID NO: 589 - DNA, 39 bp, Artificial Sequence
  Affymetrix ID 3114370
tgagaaatgcgctgttgataagatcatcgattcaggccc

SEQ ID NO: 590 - DNA, 44 bp, Artificial Sequence
  Affymetrix ID 3114664
agagagacctcatctttcatgcttgcaagtgaaatatgttacag

SEQ ID NO: 591 - DNA, 277 bp, Artificial Sequence
  Affymetrix ID 3124536
tgcagcgacttcacctaaaccgtggcactgaatgctctgccatgagccacaagcagcacagtgatcatcacccacaaggacaggttgctgggatgaggcaccctttcctttcatgtttaggttcttctcacctgcatttcactttcctaaaggtcccagccacacagcgttcttttagggattaaagtagtaggaaaaaaataaagagaacacaacatccatccttaaagaaaaaaagtaaatccactttatggtggacttcagctatggacaaatt

SEQ ID NO: 592 - DNA, 98 bp, Artificial Sequence
  Affymetrix ID 3126094
cacgattaactgtgaaatgtacgtcatttgtgcaagaccgtttgtccacttcattttgtataatcacagttgtgttcctgacactcaataaacagtca

SEQ ID NO: 593 - DNA, 34 bp, Artificial Sequence
  Affymetrix ID 3128680
gaaaaggcaatgaaagtgctcaagaaacaaaaga

SEQ ID NO: 594 - DNA, 40 bp, Artificial Sequence
  Affymetrix ID 3129955
gtatgttcaaactcagacacgaaaaccagaactgcatcag

SEQ ID NO: 595 - DNA, 31 bp, Artificial Sequence
  Affymetrix ID 3131973
aatgggtatccatcatcaatcagtgtgtatg

SEQ ID NO: 596 - DNA, 30 bp, Artificial Sequence
  Affymetrix ID 3132336
ctgtatgagtattttcagtagggggaataa

SEQ ID NO: 597 - DNA, 32 bp, Artificial Sequence
  Affymetrix ID 3133528
ttctcatgcaaagcacgtcagtagaacagggg

SEQ ID NO: 598 - DNA, 141 bp, Artificial Sequence
  Affymetrix ID 3134184
tttctaatatggtggcgaaaaatgcagaaatgcataaaaataaactgcagtactttatggagcagttttatggaatcatcagaaatgtggattcgaacaacaaggagttatctattgctatccgtggatatggactttttg

SEQ ID NO: 599 - DNA, 56 bp, Artificial Sequence
  Affymetrix ID 3136352
gtgcctcatttgaccagttctggtggtatttcgtcttttagccttggtactccagt

SEQ ID NO: 600 - DNA, 486 bp, Artificial Sequence
  Affymetrix ID 3137586
ggggtagctcttgattttgttaacattaagataaggtgactttttccccctgcttttaggattaaaatcaaagatacttctatatttttatcactatagatcatagttattatacaatgtagtgagtcctgcatgggtactcgatgtgtaatgaaacctgaaataataagataataagaaaagcaataattttctaaagctgtgctgtcggtgatacagagatgatactcaaattataataaaactcttcattttgtgaattatagaagctactttttataaagccatatttttttagggaaactaaggagtgacatagaactgatgaatgagtaaaagtaagttttgctggatttttgtagaactctggacgttgaggattcattatgctgtggttaactttaaatatttttgaattccaaatatctgaattaatgagccttgtctttacaaatatgtgccattgtgcaacatcggtggattttc

SEQ ID NO: 601 - DNA, 104 bp, Artificial Sequence
  Affymetrix ID 3138886
agaaagcctttgaggtgaaggtgtatgaaagtcatcataacagatgttttccaaaaacttgtagaaggttgtgaaaaaactactaggatcacgcggcatgtatt

SEQ ID NO: 602 - DNA, 133 bp, Artificial Sequence
  Affymetrix ID 3138980
agtactttacctgaaagacagtatgagaaaaatattcaagtaacactttaaaaccagttacccaaaatctgattagaagtataaggtgctctgaagtgtcctaaatattaatatcctgtaataaagctcttta

SEQ ID NO: 603 - DNA, 76 bp, Artificial Sequence
  Affymetrix ID 3139053
tgtctgcagtgaagcactaagttacttcctcactctaacatcagaaagtcatcgagaagcctggactaacttactg

SEQ ID NO: 604 - DNA, 85 bp, Artificial Sequence
  Affymetrix ID 3139926
aattaacaggcgaatgcacttctccaaaacaaaacacagcaagtttaatgaatctggtcagcttagtgcgttctacctttttgcc

SEQ ID NO: 605 - DNA, 29 bp, Artificial Sequence
  Affymetrix ID 3141863
catgtgaaaagcttaataaattccatcta

SEQ ID NO: 606 - DNA, 83 bp, Artificial Sequence
  Affymetrix ID 3145023
gcagttatctggagacatctgagagaatatttttatctgaagaaagcagaatttgtttgatacctaacaagatttcaataaaa

SEQ ID NO: 607 - DNA, 49 bp, Artificial Sequence
  Affymetrix ID 3145956
gtgactctgacatcattagaagcatgccagaacagactggtgaaaagta

SEQ ID NO: 608 - DNA, 125 bp, Artificial Sequence
  Affymetrix ID 3145966
tcgctggagtcgatcaactctaggctccaactcgttatgaaaagtgggaagtacgtcctggggtacaagcagactctgaagatgatcagacaaggcaaagcgaaattggtcattctcgctaacaa

SEQ ID NO: 609 - DNA, 70 bp, Artificial Sequence
  Affymetrix ID 3146538
gactgcagatgattctaggttttgattaaaagtgtggtttggggaagcaactttaccctttattgtgtgt

SEQ ID NO: 610 - DNA, 205 bp, Artificial Sequence
  Affymetrix ID 3146566
cattatggtgctactgagcgttttcttttggtaaaaagaaaaatgccatgggctgcagtcttcttccatcacttttccctaccaggtccattaatatgcttataacactagtgccagttattttatttgataatgcttatggtatttgtatatttgtttgcattccaattttgtttaataatgagtgtgtaaactgcatacgtta

SEQ ID NO: 611 - DNA, 214 bp, Artificial Sequence
  Affymetrix ID 3146788
ccttatgtaccgagcaaatgccaggtctagcaaacataatgctagtcctagattacttattgatttaaaaacaaaaaaacacaaaaaaatagtaaaatataaaaacaaattaatgttttatagaccctgggaaaaagaattttcagcaaagtacaaaaatttaaagcattcctttctttaattttgtaattctttactgtggaatagctcagaa

SEQ ID NO: 612 - DNA, 25 bp, Artificial Sequence
  Affymetrix ID 3146799
aaccgtgctgcatactatcctccta

SEQ ID NO: 613 - DNA, 73 bp, Artificial Sequence
  Affymetrix ID 3147328
gaagcttctgcagttcaagcgttggttctggtcaatagtagagaagatgagcatgacagaacgacaagatctt

SEQ ID NO: 614 - DNA, 30 bp, Artificial Sequence
  Affymetrix ID 3148628
accccaaaggtcgcgataatattattgacc

SEQ ID NO: 615 - DNA, 135 bp, Artificial Sequence
  Affymetrix ID 3149768
agttaacatgaactcttgaagtcacaccagggcaactcttggaagaaatatatttgcatattgaaaagcacagaggatttctttagtgtcattgccgattttggctataacagtgtctttctagccataataaaa

SEQ ID NO: 616 - DNA, 102 bp, Artificial Sequence
  Affymetrix ID 3149773
tttggggaagaatctacagttgctgatggacagagtggatgaaatgagccaagatatagttaaatacaacacatacatgaggaatactagtaaacaacagca

SEQ ID NO: 617 - DNA, 31 bp, Artificial Sequence
  Affymetrix ID 3149864
gcagcctgcacatgacgatatggatgaggat

SEQ ID NO: 618 - DNA, 29 bp, Artificial Sequence
  Affymetrix ID 3151480
cttcagacaggatccattgctaatcatgg

SEQ ID NO: 619 - DNA, 166 bp, Artificial Sequence
  Affymetrix ID 3152560
ttggctataatacttgtgactcatgaagaattatgttgacaaacaggataaattccacatgcattttatttcccagtgagttgtataaactttatttttgttgaaggttgtatgttaaatcaatgttacattcttatatcacttcttgagaaggaagttccgattt

SEQ ID NO: 620 - DNA, 30 bp, Artificial Sequence
  Affymetrix ID 3153532
tttgacaaagcctggaaagattatgagaca

SEQ ID NO: 621 - DNA, 72 bp, Artificial Sequence
  Affymetrix ID 3157388
cgacgagccgtgttgaaacttcttttgtatgtgtgtgtgtttttttcactattaaagcagtactggggaatt

SEQ ID NO: 622 - DNA, 34 bp, Artificial Sequence
  Affymetrix ID 3157461
gtgttttatgttcagcaataaaggttctatccgt

SEQ ID NO: 623 - DNA, 79 bp, Artificial Sequence
  Affymetrix ID 3157723
cctgggccttcggagcctcctgcctgcaccctccacctcttctaaaccatgatgtggcacattttggtgttaataaaac

SEQ ID NO: 624 - DNA, 93 bp, Artificial Sequence
  Affymetrix ID 3157847
ggaaaccctcatgctaaactgtctgattatgcatggttctaaatggtttcagtggcaaatacataacattgtactactgattaaactgaactt

SEQ ID NO: 625 - DNA, 537 bp, Artificial Sequence
  Affymetrix ID 3164299
ttgcactgcacacggaagttttattcttgtatatttagatttgtatgcttgtgagtaaaaatgtgcatttgtaaatactgtgtttttaggttgaatcaattaagtcttaaaactgtaaatttattaagcttgttgccagtaggtttaagaaaatcatgatctcacatgcctcactttgacatttatcatgccttttattaaaaatatcccttaggaacagtaagtttgccttaactctctgcatggtttaaaaagccattggttttgtgtgtgtttacagtaattatgcagatgactaaatgagttgacacagtaaaattacttccatatggtacagtaacaacctaactcccttctacctgatgtaaccatttacaaattatagttattgtaccagtctttcaacatttcagggttaatctgacctatgcagaattctagtgtgaatttattgtgaaaatgctaaatgaaacaatttgtcagaaattatgtacaattttcattaccttcaaaatggatcttttgcactgtctgaga

SEQ ID NO: 626 - DNA, 35 bp, Artificial Sequence
  Affymetrix ID 3165138
tggccaaaaaaatcgcaactttgatattccaaaag

SEQ ID NO: 627 - DNA, 39 bp, Artificial Sequence
  Affymetrix ID 3165799
taaagatgataatcaggaaatagccagcatggaaagaca

SEQ ID NO: 628 - DNA, 31 bp, Artificial Sequence
  Affymetrix ID 3166029
attacccaagaatctcagttaccaatgaaag

SEQ ID NO: 629 - DNA, 96 bp, Artificial Sequence
  Affymetrix ID 3166735
gggccagtgaataacactcactgctggcatttaatgtgcagtagtgaatgagtgaaggactgtaatcataatatgctcactacttgctcttgtttt

SEQ ID NO: 630 - DNA, 39 bp, Artificial Sequence
  Affymetrix ID 3167990
gtgtttccgtcagtacacgaaggatatcggtttcattaa

SEQ ID NO: 631 - DNA, 28 bp, Artificial Sequence
  Affymetrix ID 3168127
ttccaatcagcagtatcatggattaaat

SEQ ID NO: 632 - DNA, 148 bp, Artificial Sequence
  Affymetrix ID 3169011
gcccttctagttggagccaagtatgggattagccaggttatcaatagcaaactcaacatcatgtccacattggcagagtttgcaaacatcagccgtgtagagctaacggaagagtctgagaaagtgagcgtcgtcaaagtgtatcttc

SEQ ID NO: 633 - DNA, 95 bp, Artificial Sequence
  Affymetrix ID 3174268
gtctacaatggccctcatatgctggaaccaaacacatggaatttaaaggctttccaaaggcggcggcgccgtattacattcactcaaccttctta

SEQ ID NO: 634 - DNA, 40 bp, Artificial Sequence
  Affymetrix ID 3174441
attggtcctcggtaagtgccttttgataaatgatctcaaa

SEQ ID NO: 635 - DNA, 52 bp, Artificial Sequence
  Affymetrix ID 3175632
tacaaccctggataacacaatggaagacctgttaacgctggaatatgtaaag

SEQ ID NO: 636 - DNA, 216 bp, Artificial Sequence
  Affymetrix ID 3175657
gccactggatcttcagctgacttcgtaaaggatctagcaccatttatgattttaaattcccttggacttactatttctgtttcgccaagtgattcttttagtgtactcaacattcctatggcaaaatcatatgtattgaaaaatggagaaagtttaagtatggattatatccgaaccaaggacaatgatcatttcaatgcaatgaccagcctaagc

SEQ ID NO: 637 - DNA, 62 bp, Artificial Sequence
  Affymetrix ID 3178635
tcctaaaaatgttagtataccatcttctgaagctttatcttcggatccttcctacaacaaag

SEQ ID NO: 638 - DNA, 33 bp, Artificial Sequence
  Affymetrix ID 3183178
ggtagataatctttcgtatgcaaacttttccct

SEQ ID NO: 639 - DNA, 30 bp, Artificial Sequence
  Affymetrix ID 3190449
taggccctcaataaaagccatttgcacttt

SEQ ID NO: 640 - DNA, 92 bp, Artificial Sequence
  Affymetrix ID 3192674
tatggattcgatttgggtatgacccccgaaaaaacccagatgccaagatttatcaagtcctcgatttccgaatccgttgtggaatgaaacac

SEQ ID NO: 641 - DNA, 38 bp, Artificial Sequence
  Affymetrix ID 3197644
taattgttgccttacaatgttactgaaataaactgttt

SEQ ID NO: 642 - DNA, 28 bp, Artificial Sequence
  Affymetrix ID 3199457
agaaatacagcagacgaacatatttatt

SEQ ID NO: 643 - DNA, 384 bp, Artificial Sequence
  Affymetrix ID 3199485
ttgcttcccgaacacttaattttgaaaaatttcaaacagaagcattggaagaattttgtttaacatcggaatacctatgacttagattcttacctaacattttaacaaatttcagcacatactcatctgtctttctgtccactatgttattaatatgggggattcatttcaaagtaaattgcagatgtcagtatgcttcctctctacataattcagtatacatattgctaactagatctctgtattggtttacagattcttaaaggtaaatttacgtaccatgacatgcaacaatcttaagtgtacatatgctgagttttgataaattcatctttgtaactcagactccattatcaatagaaacatgcttgttagcaccacaga

SEQ ID NO: 644 - DNA, 34 bp, Artificial Sequence
  Affymetrix ID 3200638
atcagttaagcaggtacgaaacttgagatctgaa

SEQ ID NO: 645 - DNA, 62 bp, Artificial Sequence
  Affymetrix ID 3200711
tgaggcatgtgagatcatgattttcttaaacctactggcaacaagcttaataaatttacagt

SEQ ID NO: 646 - DNA, 30 bp, Artificial Sequence
  Affymetrix ID 3200718
gaagaagcagcgtaccaagaaaaataaaga

SEQ ID NO: 647 - DNA, 37 bp, Artificial Sequence
  Affymetrix ID 3200725
cattgtggatgcaaatctgagcgttctcaacttggtt

SEQ ID NO: 648 - DNA, 67 bp, Artificial Sequence
  Affymetrix ID 3203822
caggatgaatgcatagtggccctacatgattgtaatggagatgtgaacaaagctatcaatatattgc

SEQ ID NO: 649 - DNA, 28 bp, Artificial Sequence
  Affymetrix ID 3205039
ggcagtacatatgcagcagaccgtaacc

SEQ ID NO: 650 - DNA, 30 bp, Artificial Sequence
  Affymetrix ID 3205424
gtgactgatggatttgcactgattaggaaa

SEQ ID NO: 651 - DNA, 386 bp, Artificial Sequence
  Affymetrix ID 3212146
cacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggc

SEQ ID NO: 652 - DNA, 147 bp, Artificial Sequence
  Affymetrix ID 3212163
tgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaatta

SEQ ID NO: 653 - DNA, 29 bp, Artificial Sequence
  Affymetrix ID 3214671
gtatgtttctgtgttaccaacaacagcag

SEQ ID NO: 654 - DNA, 32 bp, Artificial Sequence
  Affymetrix ID 3214699
tgacaagaaaacagagagtgcagtatctcaga

SEQ ID NO: 655 - DNA, 36 bp, Artificial Sequence
  Affymetrix ID 3219683
gctgtttcttagctaacttgctttgacaacctactg

SEQ ID NO: 656 - DNA, 83 bp, Artificial Sequence
  Affymetrix ID 3219711
tctaggagataaggtgaacaatactgctgtaattgaaaagcaagtactagaactttgggacagactgtatcactcttggtttg

SEQ ID NO: 657 - DNA, 66 bp, Artificial Sequence
  Affymetrix ID 3219891
tctgcctttgtgaataccgtgttaaatttttttaaatttgtgacaggattgtagcaaattattatt

SEQ ID NO: 658 - DNA, 28 bp, Artificial Sequence
  Affymetrix ID 3220159
tatgaagacataaacccagttgccatct

SEQ ID NO: 659 - DNA, 146 bp, Artificial Sequence
  Affymetrix ID 3222054
ttatcgatacaccaagtaatacatgtaacaagttcttgaattctatcatctagtaattttgattaagagaaactaaaagcagcccaaacaattccactagtattcactgttctaaccattagcaagaatggactactttaaggctg

SEQ ID NO: 660 - DNA, 43 bp, Artificial Sequence
  Affymetrix ID 3222984
gtacatcaaagattacatgaaatcaatcaaaggcaaacttgaa

SEQ ID NO: 661 - DNA, 355 bp, Artificial Sequence
  Affymetrix ID 3223876
ctcctgcatggtaaatctagactttttttcccccttgtcctcgtgataagtatgtcaccaatatatgatttaaaccgagcactgatgctggacttcatgatttttaccctccctttggcaaggctttgtctcactgtacggtttaatttggtgatatcttaagcctttcttcccatccttaactgttcaagtatgtctgttgtaaccaataagtttattgctgtgaaattacttctgatggtagagaaggggttctataactgcttttgttttgttttggataaatttcctgttgtgtgggtggcatttttcttaacgagatttgcttctgtcttagcctcacacagggaaaata

SEQ ID NO: 662 - DNA, 45 bp, Artificial Sequence
  Affymetrix ID 3225580
tacactaattattctgacatgaagggaaatgatctaacaagtgaa

SEQ ID NO: 663 - DNA, 29 bp, Artificial Sequence
  Affymetrix ID 3226470
aaccaagaaattacggatcagttggaaga

SEQ ID NO: 664 - DNA, 28 bp, Artificial Sequence
  Affymetrix ID 3227646
ctttggagggagaatcaataaataacaa

SEQ ID NO: 665 - DNA, 45 bp, Artificial Sequence
  Affymetrix ID 3233387
gatacacggtatctatttggagcaacggtttttgtaactaatgtg

SEQ ID NO: 666 - DNA, 85 bp, Artificial Sequence
  Affymetrix ID 3234175
attttgtttttagcttactgctgcctttgtccgaagaaactgttcctccattatttgaattactgaagacagcaagatatttgta

SEQ ID NO: 667 - DNA, 94 bp, Artificial Sequence
  Affymetrix ID 3235500
tatattcatgtacattcacctggggaaaaaaacggagggactttgctacttgtaaaaataacataataaatagatcttaaacataggaaaacca

SEQ ID NO: 668 - DNA, 28 bp, Artificial Sequence
  Affymetrix ID 3237000
cactcagtgcagcaatatattaccagca

SEQ ID NO: 669 - DNA, 38 bp, Artificial Sequence
  Affymetrix ID 3237001
gcagtttttcaggagcgcaagatagatttggaatagga

SEQ ID NO: 670 - DNA, 656 bp, Artificial Sequence
  Affymetrix ID 3239927
ttgcttgtttcacatggagaccttggagactcaattcacgttaagacacctaagtacgagtcctccaggtaaatattacacaaatgggaagcatcttgaatttttaagtatatttcaatacataaatttttatgcatgctttaaacaaacagtattttttttaaatgagagaatctaacaaaaaaagtctgaccagcaccagcatttaaattttctgattttaatattagtctgacatagcgttagtaaccatgctgcactgaaacatgtaatggtacaatctgaatcatgattcgttaaatattataccccacttcccccagaatatttaggctggtcataaagttaaaaatgtgtaagtaagtacataagcataatcagttatggacagcttcttgtataaattgctattcagcaatacataaactgcctcaaagatttatgcttacaggtagacattcaatttaccaataaaacagcatgttctgaaaatatgggcacattttaaaacatattaagacagttctgttaaccataatagtcccacagtatgactgagtaataagaatctacttcaaaagaaaaaaaaaaattaatcagtatagtgcatgattgattcaacatagttcccagggaacagaccagtcactcgattg

SEQ ID NO: 671 - DNA, 152 bp, Artificial Sequence
  Affymetrix ID 3239982
ctgtagatcctataacagcatgttcatattcacaaaatctgaatgaaaagtaaacacaacttggcatttctgacgcagggtaacacaaagttcacttcggaggggactgagatttttgtttaattttgcttttgaaaccaagcacagttata

SEQ ID NO: 672 - DNA, 30 bp, Artificial Sequence
  Affymetrix ID 3240135
ttgtacagactaataaatctttttcacagt

SEQ ID NO: 673 - DNA, 81 bp, Artificial Sequence
  Affymetrix ID 3240409
ctgcagtctataattcataagtttcttactgctggaccatctgctttcaacataacgtctctgatttctcaagctgctcag

SEQ ID NO: 674 - DNA, 31 bp, Artificial Sequence
  Affymetrix ID 3240425
ggaaacagagctagtcagatgtgaattgtat

SEQ ID NO: 675 - DNA, 288 bp, Artificial Sequence
  Affymetrix ID 3241499
ctggatatattttatgggcatgtagtgttgttaagtggtaaaatttaaagacatttaacctaaatgttccattttctcttaagaaaaaaaaaggaagacgttttaataggaaaagaaaaacaagaagcagtagcagctaggtaatttaaactgagattaaaaaataaacatacacaaaaaatacaaaaagtacagtcctataaggtacagttagcttggcacagtaaagactaaatttaagacacgatagacaaactgtgtaataaatagggccacagttgtaaactg

SEQ ID NO: 676 - DNA, 63 bp, Artificial Sequence
  Affymetrix ID 3241506
tcctcctgatccaacatttgcgtcttcagtttctctaccagttgactttgctggttaatttct

SEQ ID NO: 677 - DNA, 25 bp, Artificial Sequence
  Affymetrix ID 3243292
aagccagagctgacaatgacactct

SEQ ID NO: 678 - DNA, 32 bp, Artificial Sequence
  Affymetrix ID 3250201
tgtgcaaccatgatgtataataaagaatttga

SEQ ID NO: 679 - DNA, 72 bp, Artificial Sequence
  Affymetrix ID 3251409
cctccccgggaggagtgccatctgggtcttccatctagaactgtttacatgaagataagatactcactgttc

SEQ ID NO: 680 - DNA, 25 bp, Artificial Sequence
  Affymetrix ID 3252608
gttagctggtttctagttggttatc

SEQ ID NO: 681 - DNA, 88 bp, Artificial Sequence
  Affymetrix ID 3253445
tcaccccgggaaggcgacagtgcctaagacagaaattcgggaaaaactagccaaaatgtacaagaccacaccggatgtcatctttgta

SEQ ID NO: 682 - DNA, 106 bp, Artificial Sequence
  Affymetrix ID 3253863
taggtgttcatgtggttattttgtatttaaagatcaaattatttgactattgctagacatttctatactctgttgtaacactgaggtatctcatttgcccatgtta

SEQ ID NO: 683 - DNA, 31 bp, Artificial Sequence
  Affymetrix ID 3255269
catatttaaatgttccggtaatgtgatgcct

SEQ ID NO: 684 - DNA, 39 bp, Artificial Sequence
  Affymetrix ID 3257404
cattcttctactctgtatgcccatgaggtcaattgattt

SEQ ID NO: 685 - DNA, 46 bp, Artificial Sequence
  Affymetrix ID 3258021
tagctttgacaaccatactgtaacattaaacctagcattccacaag

SEQ ID NO: 686 - DNA, 53 bp, Artificial Sequence
  Affymetrix ID 3259678
ctgtctttgtcacgaaagccgtttttcctcacattttccagtaggcagataac

SEQ ID NO: 687 - DNA, 32 bp, Artificial Sequence
  Affymetrix ID 3261550
ctgctgcatagtttcggtcatctaaaggaggt

SEQ ID NO: 688 - DNA, 107 bp, Artificial Sequence
  Affymetrix ID 3262470
ttgggagctcgaagaacgacacttacaagaaaaacaccagctgctcaaacagcagcttaaagatcagtatttcatgcaaagacatcagctacttaagcgccacgaga

SEQ ID NO: 689 - DNA, 75 bp, Artificial Sequence
  Affymetrix ID 3263804
tttctgctaagcgagagactagtggagaaaaatccagacaattaagagatgctcagcaggatgcaagagataaaa

SEQ ID NO: 690 - DNA, 29 bp, Artificial Sequence
  Affymetrix ID 3264697
aaagcatcaggactccaaaaaggaagaag

SEQ ID NO: 691 - DNA, 34 bp, Artificial Sequence
  Affymetrix ID 3264726
ctgtagcatgccgttctggattaataaaagcaac

SEQ ID NO: 692 - DNA, 29 bp, Artificial Sequence
  Affymetrix ID 3275175
tcatatggatcgaaacccttactacggag

SEQ ID NO: 693 - DNA, 35 bp, Artificial Sequence
  Affymetrix ID 3276455
aaatgactttctagaacttctcaggagacgctttg

SEQ ID NO: 694 - DNA, 88 bp, Artificial Sequence
  Affymetrix ID 3277709
agccagcactggatctcatctcaagctcatagtagatgctttcctacagcagttacccaactgtgtcaaccgagatctgatagacaag

SEQ ID NO: 695 - DNA, 197 bp, Artificial Sequence
  Affymetrix ID 3279906
gtcggttgttaagaactagagcttattcctattccaaatctatcttgcgctcctgaaaaactgcagaaaggcacttgaaagctgtttctttaagatatggatttcttttttattcttgctggtaatatattgctgcactgagtgtgtgcaatttttattcaaggtcatcgtgatgctgagaagtttcgttgataacc

SEQ ID NO: 696 - DNA, 42 bp, Artificial Sequence
  Affymetrix ID 3280961
gtacagcgttcaaaatctgtggaaccttataagcactcatga

SEQ ID NO: 697 - DNA, 46 bp, Artificial Sequence
  Affymetrix ID 3282022
cagtgcagcatggttactaacgctatgtcagactaatattaaaatc

SEQ ID NO: 698 - DNA, 31 bp, Artificial Sequence
  Affymetrix ID 3282170
atgacttagaagtagcatcagaagaagagca

SEQ ID NO: 699 - DNA, 56 bp, Artificial Sequence
  Affymetrix ID 3282215
aacacattgtattggtattacaaccagattatattcttgacgttgacttcattaaa

SEQ ID NO: 700 - DNA, 78 bp, Artificial Sequence
  Affymetrix ID 3282220
tgtcacatttatccagtttgggttattctcattatgacacctattgcaaattagcatcccatggcaaatatattttga

SEQ ID NO: 701 - DNA, 29 bp, Artificial Sequence
  Affymetrix ID 3282254
ttcttcccattggcatacatcccatgtct

SEQ ID NO: 702 - DNA, 84 bp, Artificial Sequence
  Affymetrix ID 3284025
tgcacagagcgttttgtatgtagtccagatgaagttatggataccatagatgaaggaaaatccaacagacatgtagcagttaca

SEQ ID NO: 703 - DNA, 198 bp, Artificial Sequence
  Affymetrix ID 3284217
tgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaa

SEQ ID NO: 704 - DNA, 45 bp, Artificial Sequence
  Affymetrix ID 3284603
gcacgcaaataacggatgcttcatgtcgcgcaataaaagacattt

SEQ ID NO: 705 - DNA, 50 bp, Artificial Sequence
  Affymetrix ID 3284665
aagcgattcaaaacatgtcttgaacagcacatattgcacagttgttgttt

SEQ ID NO: 706 - DNA, 78 bp, Artificial Sequence
  Affymetrix ID 3294341
cattacataatttggctagagcataataatttgattttctggtttttgaaaatttgagttgcaataaaaggatatttc

SEQ ID NO: 707 - DNA, 39 bp, Artificial Sequence
  Affymetrix ID 3294501
tttataaacagtagcacacaataaattccatgcatgggc

SEQ ID NO: 708 - DNA, 118 bp, Artificial Sequence
  Affymetrix ID 3298980
tgactcaaacagtgaatgcctatagaaataataaatgaaaaaaactagtatttttatatcataaaacaatgtcatttatagcttatcattcatgtattgtccagcagacattaaaagc

SEQ ID NO: 709 - DNA, 33 bp, Artificial Sequence
  Affymetrix ID 3301222
gtctgacactgattttgtttttactcaataaat

SEQ ID NO: 710 - DNA, 145 bp, Artificial Sequence
  Affymetrix ID 3301703
gccatcaggcaagaaggtataaagaggtggttttggaaaaatctcattctgctgttttactgaatcggagatattcaacttagtgctctgttgctcagatagcatgttaggaataagaacagctttcacagaactcgttggtttc

SEQ ID NO: 711 - DNA, 35 bp, Artificial Sequence
  Affymetrix ID 3301858
cctagtgtgttaatttatctgaatttggattttta

SEQ ID NO: 712 - DNA, 241 bp, Artificial Sequence
  Affymetrix ID 3301863
gtcgacctattgtgggacatttttatatattgaatctgggttcttttttgactttttttttttcccaatccaacagcatccttttttttaaaagagagaattagaaaatattaaatcctgcatgtaatatatctgctgtcatcttagttggaccaacttcccatttatttatcttaaaactatacagttacatcttaattccatccaaagaagatacagtttgaagacagaagtgtactct

SEQ ID NO: 713 - DNA, 516 bp, Artificial Sequence
  Affymetrix ID 3302047
atacacaacattacagctgggaatgtttctccaaaatgtcatggtcaaataatacaatggaaccattaaatcttacacatgcacgaaagaacaagcgcttttgacatacaatgcaaaaaaaaaaaaaaggggggggaccacatggattaaaattttaagtactcatcacatacattaagacacagttttagtccagtcaaaaatcagaactgcattaaaaaatttaatgtagtgcaatcaaaccaaaaaacttaatttgtgatcataagtgctctactacataaacattgatcatagcaaggaacaaaaagttcaaatcacacagtacaaaaaaatctgtaaataaatataaactacagtacaagagaaatattaaattatacaattccatcaaactgtaaacagtatattgaaatgaggtccataagagtaaagggggttcctgttttgttgtatgacacttgaacttctagaaatctgaaagataccattttccatatctccaaaggctatggg

SEQ ID NO: 714 - DNA, 64 bp, Artificial Sequence
  Affymetrix ID 3304013
tattgattgtttggatcagagctcagttcctgtagaaaacgaactgtaaaagaccatgcaagag

SEQ ID NO: 715 - DNA, 85 bp, Artificial Sequence
  Affymetrix ID 3304626
tttgacctattctgtagtacaactgtaatagctattggtcttcaagtgggtttagatttggtgacatcagtttgatattctctta

SEQ ID NO: 716 - DNA, 91 bp, Artificial Sequence
  Affymetrix ID 3304652
ccagaaactagagaacagtatccaaataaatttatccagcgagatgatactgaaagattttacattctgaacacactattcaacctaccag

SEQ ID NO: 717 - DNA, 35 bp, Artificial Sequence
  Affymetrix ID 3307940
actgtcatactcacaacttcgtgaataaagttgtg

SEQ ID NO: 718 - DNA, 52 bp, Artificial Sequence
  Affymetrix ID 3308532
ctccatccctgatcagcagcttttgaaagctttagacgaaaatgcaaaactc

SEQ ID NO: 719 - DNA, 102 bp, Artificial Sequence
  Affymetrix ID 3309267
gaccttgaggaattggatccagattttatcatggctaaacaggttgaacaactggagaaagaaaagaaagaacttcaagaacgcctaaagaatcaagaaaag

SEQ ID NO: 720 - DNA, 42 bp, Artificial Sequence
  Affymetrix ID 3311304
ttctgggccgtcctagacaagcatagattgccagtttcagaa

SEQ ID NO: 721 - DNA, 50 bp, Artificial Sequence
  Affymetrix ID 3317557
tgtattcagttacaaatgtgttctaaggttaggccgagcactctccacag

SEQ ID NO: 722 - DNA, 38 bp, Artificial Sequence
  Affymetrix ID 3325714
ccgtaagaactaaaatggtctactgcaaaattgatcag

SEQ ID NO: 723 - DNA, 116 bp, Artificial Sequence
  Affymetrix ID 3325963
taaatgtgaccaagccttctgtactttgtcacaaaaagcgggttttccaggtgactattttggtgagtgtcaaaataagaatttatggtgtattactgtcgattcactttgaatta

SEQ ID NO: 724 - DNA, 322 bp, Artificial Sequence
  Affymetrix ID 3326246
ctggtgttcaacagctagcagcttatgtgattcaccccatgccacgttagtgtcacaaattttatggtttatctccagcaacatttctctagtacttgcacttattatcttttgtctaatttaaccttaactgaattctccgtttctcctggaggcatttatattcagtgataattccttcccttagatgcatagggagagtctctaaatttgatggaaatggacacttgagtagtgacttagccttatgtactctgttggaatttgtgctagcagtttgagcactagttctgtgtgcctaggaagttaatgctgcttattg

SEQ ID NO: 725 - DNA, 29 bp, Artificial Sequence
  Affymetrix ID 3326465
cttcatcaagacctctcaaggtagtgtgg

SEQ ID NO: 726 - DNA, 327 bp, Artificial Sequence
  Affymetrix ID 3329941
ctgccacatacttgaggggaaattctcaaaatcaaggatatgaacacctgctgctgcctgtatgccacctagtgtgcgaaacgaccgagctaccagttctagaacttatgagaactactctaaagagtgtggccatctatcagcacgaggtaaaccacttagacctcagcacttcaggggtggccgactagagaaaagaggccagaaaaagcttcagacaattccagtcaagcaaggtagaatgcagagctgccttctgaattgcacttgttctggaggagaagttaattcttcacttttgtcagggagtcctctacagaagtgtta

SEQ ID NO: 727 - DNA, 164 bp, Artificial Sequence
  Affymetrix ID 3331532
cattcttgcaaaagtacggattctagataattatatagccaactcatcagtagctgaaaaactagggtattctcttctctttttgaaaatgtgaatccgcctgaaaattatagatgctgtcctaattgtaacaagatgggctttagtgtgtaagctgctcatta

SEQ ID NO: 728 - DNA, 74 bp, Artificial Sequence
  Affymetrix ID 3331573
gctcagaaaaagaagttcgagcagcagcacttgtattacagacaatctggggatataaggaactgcggaagcca

SEQ ID NO: 729 - DNA, 117 bp, Artificial Sequence
  Affymetrix ID 3331613
tgtccctggtgatgttggaacattaatgatggaacatggccaaacttcagtcatgatcctgaagccatggtttcttccctgccagaaatgaaggttcagttatgaggcaaccctcta

SEQ ID NO: 730 - DNA, 31 bp, Artificial Sequence
  Affymetrix ID 3331614
gtaaggcattgtaaaagttactggatttggt

SEQ ID NO: 731 - DNA, 25 bp, Artificial Sequence
  Affymetrix ID 3333687
atcactctttatattaaaaagtaag

SEQ ID NO: 732 - DNA, 34 bp, Artificial Sequence
  Affymetrix ID 3334162
ctctgccaggcgctagacatgtacagaggttttt

SEQ ID NO: 733 - DNA, 36 bp, Artificial Sequence
  Affymetrix ID 3334515
tgctggggccctgcaattggaatgttggccagattt

SEQ ID NO: 734 - DNA, 232 bp, Artificial Sequence
  Affymetrix ID 3335173
tttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaa

SEQ ID NO: 735 - DNA, 183 bp, Artificial Sequence
  Affymetrix ID 3335176
ttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagg

SEQ ID NO: 736 - DNA, 376 bp, Artificial Sequence
  Affymetrix ID 3335178
gccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaagga

SEQ ID NO: 737 - DNA, 31 bp, Artificial Sequence
  Affymetrix ID 3335179
cttttagaaagctgtctccttatttaaataa

SEQ ID NO: 738 - DNA, 230 bp, Artificial Sequence
  Affymetrix ID 3335181
gcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgtttta

SEQ ID NO: 739 - DNA, 50 bp, Artificial Sequence
  Affymetrix ID 3335182
gaagccattcaggattttgaattgcatatgagtgcttggctcttccttct

SEQ ID NO: 740 - DNA, 197 bp, Artificial Sequence
  Affymetrix ID 3335187
attcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtgga

SEQ ID NO: 741 - DNA, 177 bp, Artificial Sequence
  Affymetrix ID 3335188
tgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcc

SEQ ID NO: 742 - DNA, 175 bp, Artificial Sequence
  Affymetrix ID 3335189
aagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttga

SEQ ID NO: 743 - DNA, 61 bp, Artificial Sequence
  Affymetrix ID 3335191
ctcacctcgatgcagccagtagcttggatccttgtgggcatgatccataatcggtttcaag

SEQ ID NO: 744 - DNA, 92 bp, Artificial Sequence
  Affymetrix ID 3335231
gtggtgaaatacatataatagaagttgactatctgaatcatttttaagtatacattcagtagtgttaagtatgtcgccattgttgtacaacc

SEQ ID NO: 745 - DNA, 1338 bp, Artificial Sequence
  Affymetrix ID 3337724
gggatcaggaccagcaacctttatattctagattctaagacattgtacagagaaattcagaagtgtaaaaatattgcacattgacaaataccaagaatttttgcgtatgtttatattgtattgttctaaataatgggtagcctgtgaaataagatcttgccacccatgtaataatagtagtaatactatagttaaaatggctgtaagaatagttttataaaagtgaatacacagatctattgtatttgaaacataactttgacaattattagtgtgaccaaagtattaggcggttttcatacatttttcaccttgtacaaaattatgaattcatttttcctccaggccgacaaggagttgtagaatgaaaatgccctctaagtgttattttggttgttctaacttacaaaagtgattttgaataagaaatatttggtgttctttttataaccagtttttgattggtaattgttttctgtattgtttaaaacggatcaaaaatgtaagtctattggtagagattaagtaaagtatttattgctacatcatagttgataaattgatgttatcgtaaagccatatgttctgttcaagtcttgtttgcttgaaatgattattcctacaagtgaaacactagactatttggagtgtatatggcttgtgttttgggattttttttttttttttttggcttttgtttttgtttgtttttttgtttcatttggtagttcatctgccttttaacccattcaccaaaatttaccttgttaacaagcatcaccaatgaacatttcagagcaatctgcatatttaacagacctaaaataaatcctattaggcaagtcagttgaaaatgctcgtgctgctaatggaattagagtgcgttcattttacaggctagtattttaaaagtagaaatcaaaatctggcaccgaagcatgctaattgtttactgtaccttgtgaggttttcactcataaatttaaaccagtgtatttttttagaactggtttgtgtatatatatagtgattatggatactaattcaatgtaatttataattttctatgtcaatacaaaaatacatcacagccttctcaaacagctcaagcaatatattgtatattgccatatcgtctggtgaaagggttaaattacttcacctcttgcacttttagatgcaaatcagtttttcatttctgtaatagaaaattattcacgtatttttacatcatttgtttttcctgaccagtatttaaaaccaaaaggatattctgaaaaatggccaacaatttttttagaagtagcatcccaagcagcgtgcctaaacattacattg

SEQ ID NO: 746 - DNA, 99 bp, Artificial Sequence
  Affymetrix ID 3338618
ttttctcatcatccttgctttaccacaatgagcaatgaggtcgggttttatatgcaacttattgtatctgaattcctgtagcacacctcataggtatga

SEQ ID NO: 747 - DNA, 34 bp, Artificial Sequence
  Affymetrix ID 3341466
tggagccttaagaggagaataaatcactaagtgc

SEQ ID NO: 748 - DNA, 120 bp, Artificial Sequence
  Affymetrix ID 3341504
gtcaacatacagattagcataagcttcaactgtcataagaaacatgttccatcaaattcagaaacagcaggtatcagtgaaactggagcaagcattttgaagacttcaacgtattggatg

SEQ ID NO: 749 - DNA, 31 bp, Artificial Sequence
  Affymetrix ID 3344853
attgtgtatatgtagcattagacaaaattat

SEQ ID NO: 750 - DNA, 40 bp, Artificial Sequence
  Affymetrix ID 3344889
ctttggatgttagctatgtcttgtgagtataaattccaat

SEQ ID NO: 751 - DNA, 29 bp, Artificial Sequence
  Affymetrix ID 3345453
gcaccttgcagatgctcaataaagtaatt

SEQ ID NO: 752 - DNA, 28 bp, Artificial Sequence
  Affymetrix ID 3347691
tagtgttagtgatgcaaacgaacctgga

SEQ ID NO: 753 - DNA, 71 bp, Artificial Sequence
  Affymetrix ID 3352533
tggaagtattttgaaccgagagtcaccaacagataagaagcagaaagttgagcgcattgcatcacatgatt

SEQ ID NO: 754 - DNA, 43 bp, Artificial Sequence
  Affymetrix ID 3352561
cacctgaaagtttctgttcctgatgaaatgtctgcagatctag

SEQ ID NO: 755 - DNA, 76 bp, Artificial Sequence
  Affymetrix ID 3352585
gcagtaaccaacctttcgccctggaaatgatcaaatctcgtcagaaaaaggattctcgatttcagacttttgtgca

SEQ ID NO: 756 - DNA, 34 bp, Artificial Sequence
  Affymetrix ID 3352925
aattattcaatggagagtttacaaagactgaata

SEQ ID NO: 757 - DNA, 41 bp, Artificial Sequence
  Affymetrix ID 3354966
cagaaattatgggttgcacagggtgccagatagattggaaa

SEQ ID NO: 758 - DNA, 257 bp, Artificial Sequence
  Affymetrix ID 3356173
gagagaccctcgcaaaatgatgtgaaaggaccagtttcttgaagtccagtgtttccacggctggatacctgtgtgtctccataaaagtcctgtcaccaaggacgttaaaggcattttattccagcgtcttctagagagcttagtgtatacagatgagggtgtccgctgctgctttccttcggaatccagtgcttccacagagattagcctgtagcttatatttgacattcttcactgtctgttgtttacctaccgta

SEQ ID NO: 759 - DNA, 32 bp, Artificial Sequence
  Affymetrix ID 3358364
tgttcatctacccatgaggaataaaaccatgc

SEQ ID NO: 760 - DNA, 87 bp, Artificial Sequence
  Affymetrix ID 3359382
tgcgtttttcacttttacactcatgaatgtaactttgccatgactcattacaagatatttgaaacaattaaagctgaggagggaaca

SEQ ID NO: 761 - DNA, 33 bp, Artificial Sequence
  Affymetrix ID 3362739
ctctcccaaacttcatgtagataaaggatttgt

SEQ ID NO: 762 - DNA, 38 bp, Artificial Sequence
  Affymetrix ID 3362742
agctaaattacaagatcgagaatggttaacagaacttt

SEQ ID NO: 763 - DNA, 204 bp, Artificial Sequence
  Affymetrix ID 3362745
gaaactgttgtgactgaatatctaaatagtggaaatgcaaatgaggctgtcaatggtgtaagagaaatgagggctcctaaacactttcttcctgagatgttaagcaaagtaatcatcctgtcactagatagaagcgatgaagataaagaaaaagcaagttctttgatcagtttactcaaacaggaagggatagccacaagtgac

SEQ ID NO: 764 - DNA, 224 bp, Artificial Sequence
  Affymetrix ID 3362763
ggaagttaggcgaatgtagtcagtcgtttggaggaaatgagtcaattattcatctaagagggaggttgactagttaaatacagatgtgcaacaaaatttactaaacagaataatttgaaatacgtggtgtgtttgacaaagtagagttagaactgatcaaccagactggtagaaagccctatactagattctgtaggaactgtgaatgagtcagtgcaaacata

SEQ ID NO: 765 - DNA, 174 bp, Artificial Sequence
  Affymetrix ID 3362773
tcctttgggtccgagttcagtcttcatttgtagcataatatacaagcactaaaatacaggcctggctccagtctgggcaaaatagcgagacccccatctctacaagcaaagcaaaacacaaaatagatgcttattctttttgagtgagacatagttgggatacagtaaacaatt

SEQ ID NO: 766 - DNA, 36 bp, Artificial Sequence
  Affymetrix ID 3362943
agtttgcatgtctcatgataaccaaatgtaagatga

SEQ ID NO: 767 - DNA, 107 bp, Artificial Sequence
  Affymetrix ID 3363519
gaagtgcagttgatatctaatttacacagtgaaactaatgatagaaaataactaatgaaaaaaaatcagagactggtttccaattgattgacacctagatctgtcag

SEQ ID NO: 768 - DNA, 94 bp, Artificial Sequence
  Affymetrix ID 3363981
acctcactgaaacctatataatggaatacatttttctttgaaagggtctgtataatcattttctagaaagtatgggtatctatactaatgtttt

SEQ ID NO: 769 - DNA, 28 bp, Artificial Sequence
  Affymetrix ID 3364729
gatgctaaattccgtctgattctaatag

SEQ ID NO: 770 - DNA, 93 bp, Artificial Sequence
  Affymetrix ID 3364770
atgtcaaaacatacctacttccagataaccacaaaacatccaaacgtaaaaccaaaatttcacgaaaaacgaggaatccgacattcaatgaaa

SEQ ID NO: 771 - DNA, 132 bp, Artificial Sequence
  Affymetrix ID 3364800
tatctgaataggtatgcacagctttgtttgccactcactgttccatggagaaaagcagcagccaatgactgtcgtttatataaagtactacattgttgacataataccacagtcttgtaagttcccttgatg

SEQ ID NO: 772 - DNA, 537 bp, Artificial Sequence
  Affymetrix ID 3368920
gcggccatccggattacatcccatatttgttgcatatttttcaaaaagtgccaatcaaagcctaattttgcatcttggctttgtcttatacagtatcagctgttaaaaagcattttacatgtgtttttaaccacagttgtttggccagatgaacagtgctgtgtcaaagctggtagccagccttatatgtgtgattaggacccatgcataatttggtatgcatctgaacgttcagtgctccaatttaactggaaaagctgtgaaatgcagttgttctgccaaaccacactccactcctttcatcttcctttctagggaaataatttagtttgtcagttatcattgaataactcgagggctgatttgtagattttatccaatgtcataaaagtcaatcttgcctttttcaagccacttcaaataactatggagattattatcaccctgtaaagtatattgttttactcctaagcaagggtgaggaatctgagtatcatgtgcaaggctcaagatgacgcttaggacagaacatgcaga

SEQ ID NO: 773 - DNA, 107 bp, Artificial Sequence
  Affymetrix ID 3371737
tttctccacacttcgatgacatgcacagtaatacagcatccacaatcaatttcattatctcccaagtagccagtggtgacatcaacacaagtatccaagctctgaca

SEQ ID NO: 774 - DNA, 42 bp, Artificial Sequence
  Affymetrix ID 3372461
tctgaccttgtaaatttgtatatatgcactaataaagctttt

SEQ ID NO: 775 - DNA, 28 bp, Artificial Sequence
  Affymetrix ID 3375309
atgaagctcgagtttcattaaatatgtt

SEQ ID NO: 776 - DNA, 108 bp, Artificial Sequence
  Affymetrix ID 3375752
ttcctaaggctgacattgagatttctggccccaaagtggacattgatgcccctgatgtcagtatcgaaggtccagatgcaaaactcaagggtccaaagttcaagatgc

SEQ ID NO: 777 - DNA, 37 bp, Artificial Sequence
  Affymetrix ID 3375863
cctcggattctgtaattggttcttatagtaacaaata

SEQ ID NO: 778 - DNA, 82 bp, Artificial Sequence
  Affymetrix ID 3376090
cgtaatttctatgatcgatacaggggagactatgatcgattttacgggcgagattatgagtacaacagatacagagactatt

SEQ ID NO: 779 - DNA, 30 bp, Artificial Sequence
  Affymetrix ID 3376775
catctgttccaggataaggcgcatggctgg

SEQ ID NO: 780 - DNA, 34 bp, Artificial Sequence
  Affymetrix ID 3377457
gttaccctggtcattaataaagctgtgactggtc

SEQ ID NO: 781 - DNA, 28 bp, Artificial Sequence
  Affymetrix ID 3377464
gtcttttgtaattctggctttctctaat

SEQ ID NO: 782 - DNA, 812 bp, Artificial Sequence
  Affymetrix ID 3377621
gctacaacatagacgcatctcgaggacactgtgctcagagaaataagccagtcacagacagacaaatacttcatgatttaactgatatgaagtgcttggaatagtcaaactcacagaaacagaaagtagaatggggttgctgaggctggagggaatggaatgtttaatgttatttaatgggtacagagtttgttttgcaagatgaaaaagttctggagattggttgtacaacaatggcgacatacttaacactactgaatgtatacttaaaaatgattcagatagtcaacttctattatatgtatttcaccacaaaaatttcttaaagtagcaagaaataaataaaattgtaaaaacctgatgttagtggctatgtagggaaatggatatcatatactcacaacaggaagtgtagctttctggagtcatttgagtctgtgaagagagttataaaaccataagtgctcactgccgaggcaatgccactccaggggatacatgtgtacactaaatttacagagatgctcacagctgcacgatttaaagtagtcaaaacagtttaacagctcaagagatgggctacatatcctttagtataagcacaatggcataaaatcatgcaatcattcattcatgatcttttttgagcacgaacaagcagatgaaagttatgctttccacagaaaaatacaatttcagagcatttatagacttctagaaacccatccatgggacttcctttgggaaggttaagaatcctgagaaaaagcctcttcaaatgtgtttgtgaactctgccggtacagggaaata

SEQ ID NO: 783 - DNA, 408 bp, Artificial Sequence
  Affymetrix ID 3377623
gctttttgcaccaacaataacaggaaaaatggaggataaggtttacaatcataagtggcaaatgtggccttcccaatgatactaagagttcatgatcattataaaggtcaatatgagaatttcacatggttagtggtcaaagaggaaaaatgcagagagaaactgtggtaaaatagaaaattataaaaaccattcgatccagcaatcccactactgggtatctacccaaaggagagtaaatcattatactaaaaagacacctgatcatagcacaattcagaattgcaaagatatggaaccaacctacatgcccatcaactgatgagtggataaagaaaggtgagtacagaaatatagactaagacaactaaaataccttaaaagtaccttataacgttggatttggca

SEQ ID NO: 784 - DNA, 675 bp, Artificial Sequence
  Affymetrix ID 3377625
accataggagttactgtctgtattgtcctagtttttgtattggataggagattcagaaatgcttaccacattattaaaaaacaattaaagaaagcaagtaaaagagagccatgttgtgtcctgaatcaaagattaagattgagatttacccagttctgtgtatttgagttccaacagagagaatattcttgccttacaggagtgttctagcctccatttaccagattttaaagcataatgaaaagtgatgatcattacattccattgcattttattaacaacaacaacaaaaactggagcctaaaatttccagaaatagatccagagagaaaatgttaagaaccatttgatctagcaatcccactcctgggtatctacccaaaggaaaagaagtcattataccagaaagacacttgatggcagtacaagtcacaattgcaaagatgtggaaccaacccatcaaccagtgagtggataaataaaatgtggtatctctacaccatggaatatgacttagcaataaaaaagaacaaaataatgtttgtggttttttttttttttgcagcaattttggatggagctggaggatattattctaagtgaagcaactcaggaatggaaaaccaaatactgtgtgttctcacttataagtgagagctaacttatgggtacaaa

SEQ ID NO: 785 - DNA, 143 bp, Artificial Sequence
  Affymetrix ID 3377632
taaaaccattaggaactggcacgtgaatagacagatcaatagttaatagctgtattagccagaaaatggtgtaaggacaacaggctaactaaccctgtcacttgttatgctaaaattaagtctagatagagtcctccaaaata

SEQ ID NO: 786 - DNA, 112 bp, Artificial Sequence
  Affymetrix ID 3377633
caaattaaatagacgtgagtggatggaaagacgtaccttattcttgcataagaagacaacggcatgaagacatcacagggaaggttaaggtaaacatgcagcctggaatagc

SEQ ID NO: 787 - DNA, 632 bp, Artificial Sequence
  Affymetrix ID 3377659
cctcattttgtccactggtgaattcaactggaagctccttctatagtctgaagaataccatctgaaagaactagtggttcccaatccccacatttaaaatggaatgtttggtttatttaaagtaaataggctattttttcttactgggtctggcttctctggcccttcgcatacgtgtgtctgctgagtgttcctgcatgtaagaattaagaccaagggaggggagagagaaacccacacataaacaatgcactaaagatcactgaactgtttaaacatttccacttgccagtttaatttcttgaagactgttgcttgtttggaatgtttcttgtcactgattttaaggttgcatctggaaaagactaaaggcttcagtcccctcccaccaccagaaatgaacaaaaagcattttacctaaaaatacaccagcaaaatgtactcagcttcaatcacaaatacgactgcttaaaactgcagaaatttcctcaacactcagcctttatcactcagctggattttttccttcaacaatcactactccaagcattggggaacacaacttttaatcatactccagtcgtttcacaatgcattctaatagcagcgggatcagaacagtactgcattta

SEQ ID NO: 788 - DNA, 760 bp, Artificial Sequence
  Affymetrix ID 3377660
ggccaagtctgttatgttcacctgaaaaagtcttagcagagaatttttgcattcccacccaaaagccctctcagccactcaaatgcctatcttctccagtctacaagttacatgttcccacccagcattacagttcttgaacatgttatttctccacttactggtttaagttggttttagtcactggataagtattataatagagctacttagctgtggttttaatacccttctgttaacaatttgcaggcaaattaatggccttttctaacatagttcaacccaccaaagacctcgacaccatcgttaccttgaaaccgattatggatcatgcccacaaggatccaagctactggctgcatcgaggtgaggggtgaagggtctgtgctagatcaaaaggcacggggtggcgatgtggcagagaagttgcttgtggggagaccttgcttttaacaggcttctggaaaagctagggaaaagtggttgcccgctttccccctttccctccccttcaagcaccgcttgagatttgggctttattattaagagctgctataaaaacagcttgcttaaatgttaagagaagcccaggggtacttcaagcattccttcggatgcttcactccagaaagagggagttgaggcaagtcctcattactaataaaaatcaggtgaggctgacacttctcttgaccttaggataatagcgctttgttgtctctcctgccacaggaaggctccatggttgtcctactttaag

SEQ ID NO: 789 - DNA, 150 bp, Artificial Sequence
  Affymetrix ID 3379122
aaccaccagaactggtcgaatgaggcacctaaaaattgtataacacagattcaggcatggattccgtgaaggaacaacacctaaacccaagagggcagctgttgcagcatccagttcatcttaagaatgtcaacaattagtcatgcaata

SEQ ID NO: 790 - DNA, 96 bp, Artificial Sequence
  Affymetrix ID 3379574
atacaggtattgtattgtagacatctgttagtctcataattcagtatggccaacacacagaaattaaaagtagaaacaaaatgagggcacacttgc

SEQ ID NO: 791 - DNA, 142 bp, Artificial Sequence
  Affymetrix ID 3382981
attttgtagaaactcggcgaaggcggtcaaggagaaatcagaaaagacaaattaactacaaagaagactcagaaagtgacggttcccagaagagtttgcgacgtggtaaagaaataaggcgagtacacaagcgaagactttc

SEQ ID NO: 792 - DNA, 152 bp, Artificial Sequence
  Affymetrix ID 3382984
agttggccgaggaaaagatatctccaccatcacaggtcatcgtgggaaagacatctctactattttggatgaagaaagaaaagaaaataaacgaccccagagggcagctgctgctcgaaggaagaaacgccggcgattaaatgatctggaca

SEQ ID NO: 793 - DNA, 32 bp, Artificial Sequence
  Affymetrix ID 3383148
cttatgctaatctgtatgttgacaccttgtaa

SEQ ID NO: 794 - DNA, 66 bp, Artificial Sequence
  Affymetrix ID 3383149
gctccagtttcactgatacctgctgtttctgaatttgatggaacatgtttcttatgacagttgaag

SEQ ID NO: 795 - DNA, 34 bp, Artificial Sequence
  Affymetrix ID 3384489
aaagcaacttatggaaacaactacagaatttaca

SEQ ID NO: 796 - DNA, 31 bp, Artificial Sequence
  Affymetrix ID 3384495
gctcatttggatgctatcaggaaagacatgg

SEQ ID NO: 797 - DNA, 130 bp, Artificial Sequence
  Affymetrix ID 3385045
ctactaaaccggtggaatctgatcttgaaaatctgagtaggtggacaaatatcctcactttctatctattgcacctaaggaatactacacagcatgtaaaagtcaatctgcatgtgcttctttgattaca

SEQ ID NO: 798 - DNA, 30 bp, Artificial Sequence
  Affymetrix ID 3385178
tgtagtcgtagccaataaattttatcagct

SEQ ID NO: 799 - DNA, 40 bp, Artificial Sequence
  Affymetrix ID 3386816
agtcagcatgtatttttggctcgaagtttctctagtgttt

SEQ ID NO: 800 - DNA, 39 bp, Artificial Sequence
  Affymetrix ID 3387176
gaatttcacaaaaagttcatggagaaatatattaaatag

SEQ ID NO: 801 - DNA, 74 bp, Artificial Sequence
  Affymetrix ID 3387436
tagccaaaagtttgctgatgttactgtacaaattggctgcctattgctttacactagaagtattgaccccctta

SEQ ID NO: 802 - DNA, 256 bp, Artificial Sequence
  Affymetrix ID 3388633
cacagcactcaggattttgggtatcttttgtagctcacataaagaacttcagtgcttttcagagctggatatatcttaattactaatgccacacagaaattatacaatcaaactagatctgaagcataatttaagaaaaacatcaacattttttgtgctttaaactgtagtagttggtctagaaacaaaatactccaagaaaaagaaaattttcaaataaaacccaaaataatagctttgcttagccctgttaggg

SEQ ID NO: 803 - DNA, 76 bp, Artificial Sequence
  Affymetrix ID 3393789
aacactgccaggcaagtttgacaagaagtaaccttcctggaatgtttcaaaatacagtcgcgttgatcaagtttta

SEQ ID NO: 804 - DNA, 25 bp, Artificial Sequence
  Affymetrix ID 3394076
ttgtttgacaaagctacctatgata

SEQ ID NO: 805 - DNA, 38 bp, Artificial Sequence
  Affymetrix ID 3394529
cctggtcattgaagcacatcggataaagactgccaccc

SEQ ID NO: 806 - DNA, 69 bp, Artificial Sequence
  Affymetrix ID 3395420
gccaaccaagtgtagatgtagcattgttccacacatttaaaacatttgaaggacctaaattcgtagcaa

SEQ ID NO: 807 - DNA, 60 bp, Artificial Sequence
  Affymetrix ID 3395427
cattgatgccaatggtatactcaatgtctctgctgtggacaagagtacgggaaaagagaa

SEQ ID NO: 808 - DNA, 171 bp, Artificial Sequence
  Affymetrix ID 3400152
ggtaagggtaaatagctagtgcctagatagagggaaaggcctaagcaaagactgtgcaagaatagattgatagaagccaagacagatgtaggattagactgtggaagggtagaaggaagctgtgtgcttgcggtggatggaatggttaacaggatggaatacccagacctc

SEQ ID NO: 809 - DNA, 109 bp, Artificial Sequence
  Affymetrix ID 3402624
aatgcctacacgctctttaaaatgcaaggctttctcttaaattagcctaactgaactgcgttgagctgcttcaactttggaatatatgtttgccaatctccttgttttc

SEQ ID NO: 810 - DNA, 66 bp, Artificial Sequence
  Affymetrix ID 3402667
ctttatttcatttagccgctcagtggctagagccgctgggttcctggtattaaaaagatgccaaca

SEQ ID NO: 811 - DNA, 31 bp, Artificial Sequence
  Affymetrix ID 3402720
ctatatataatatgacgcagaaataaatcta

SEQ ID NO: 812 - DNA, 230 bp, Artificial Sequence
  Affymetrix ID 3406062
accttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccag

SEQ ID NO: 813 - DNA, 84 bp, Artificial Sequence
  Affymetrix ID 3407177
cagacatttcaagctccactatgtgcagtaaaaacaaaagtaggctacagtctgtgccatgttgatgtacaatttcgaaattat

SEQ ID NO: 814 - DNA, 71 bp, Artificial Sequence
  Affymetrix ID 3407275
ttattgcctcatgttcttgtaagtcattcttaattgaccaatgattgtagaccttgcttgagtattttttc

SEQ ID NO: 815 - DNA, 47 bp, Artificial Sequence
  Affymetrix ID 3409292
gattttgctgcccgttcccccagtgccagcattacagatgaagactc

SEQ ID NO: 816 - DNA, 46 bp, Artificial Sequence
  Affymetrix ID 3412651
tcatatgcaattcccgatgtatcaaaacgcacaaagagcaagatcc

SEQ ID NO: 817 - DNA, 85 bp, Artificial Sequence
  Affymetrix ID 3414226
tcagctcaggctgcattctaactcatactgtgaagacaaaggtgtttttgattcagaaatatatgaaatctgcatagtcttaatt

SEQ ID NO: 818 - DNA, 42 bp, Artificial Sequence
  Affymetrix ID 3416068
gtttggatgcatctgctcagactacttctcatgaactcacca

SEQ ID NO: 819 - DNA, 325 bp, Artificial Sequence
  Affymetrix ID 3416501
gttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgta

SEQ ID NO: 820 - DNA, 28 bp, Artificial Sequence
  Affymetrix ID 3417160
tagctcattaaaaaaatgtgcctagttt

SEQ ID NO: 821 - DNA, 109 bp, Artificial Sequence
  Affymetrix ID 3417666
tatatcttcaatcaaggctcttgtgcagcctacacagaaaaatgaagctttttgggttaggggcaaggagagagacagtacagaggacaaagacccctcacgatgaatg

SEQ ID NO: 822 - DNA, 32 bp, Artificial Sequence
  Affymetrix ID 3418209
gaaagtcactttaatagatagggacagtaata

SEQ ID NO: 823 - DNA, 83 bp, Artificial Sequence
  Affymetrix ID 3419262
tccttattggctagtgggcatgacagaaatgactcggacgtttggcctcgaattacttgagtcagtcctcaatgattttccgc

SEQ ID NO: 824 - DNA, 27 bp, Artificial Sequence
  Affymetrix ID 3419622
gttttctccatttggtacaatcaccag

SEQ ID NO: 825 - DNA, 33 bp, Artificial Sequence
  Affymetrix ID 3421154
ttgtataacttagcatcagtagttcaataaatt

SEQ ID NO: 826 - DNA, 59 bp, Artificial Sequence
  Affymetrix ID 3421223
tgtacttttcagaatattatcgtgacactttcaacatgtagggatatcagcgtttctct

SEQ ID NO: 827 - DNA, 123 bp, Artificial Sequence
  Affymetrix ID 3421350
cacttgatatatggaggcagtgacagctatttttacaaaatttaaatctgcaaatggattcaacatgtttatgggttattaaaattgtctgatttcttaggttctttatagtacacgtgttga

SEQ ID NO: 828 - DNA, 138 bp, Artificial Sequence
  Affymetrix ID 3421492
ctcagggaaagtcttgtgttcagcaatatctaaagataatgttactatgacaacatttttactgtcctttaaagcattgcaatagcgtttttggatatgcctcaatctaatcttgcgttcagtgaattaaacatagta

SEQ ID NO: 829 - DNA, 60 bp, Artificial Sequence
  Affymetrix ID 3421652
ttcccgggtaagagttgactctacagcaaaggttgcagaaatagaacatgcggaaaagga

SEQ ID NO: 830 - DNA, 79 bp, Artificial Sequence
  Affymetrix ID 3421668
ggtgcccattatatccttaagtttggatatttagctgaccttcgctttaacataggtctaatttatttgccgtgtcatt

SEQ ID NO: 831 - DNA, 46 bp, Artificial Sequence
  Affymetrix ID 3427811
cttcctgattaaattgtagatgtagactttacaatataattcaata

SEQ ID NO: 832 - DNA, 32 bp, Artificial Sequence
  Affymetrix ID 3427812
tttgtcgttgcaataaatggttttcagatagc

SEQ ID NO: 833 - DNA, 67 bp, Artificial Sequence
  Affymetrix ID 3428711
agagacttccgaacacttgctatctcttgctgctgctgtttcatggaaggagatattaaacatttgt

SEQ ID NO: 834 - DNA, 32 bp, Artificial Sequence
  Affymetrix ID 3428712
tttaagtgttatacctatttcagcaaataaaa

SEQ ID NO: 835 - DNA, 128 bp, Artificial Sequence
  Affymetrix ID 3429794
gcgactttctgtgcatactcatttaaagctggatgaccgaaaccctttcaaagttggcatgaaagacctggctctttttttctctctgaatccaattcggtttttcaatcgtttcattgacattcggg

SEQ ID NO: 836 - DNA, 151 bp, Artificial Sequence
  Affymetrix ID 3430575
tgttccatgcgtggcagcaaccatgcagagtgactgaaacacaattcatttctgactgacattcctttctgcaactgcggtggcaccacaaatatccggtctttgtgcttgctcttcagatggatggtttgtaaggctcttgttgcatttc

SEQ ID NO: 837 - DNA, 37 bp, Artificial Sequence
  Affymetrix ID 3431352
atgtccagtaatggtgaaataaaatcctgcttcgaca

SEQ ID NO: 838 - DNA, 777 bp, Artificial Sequence
  Affymetrix ID 3431686
ccacacggtattgtacacggtcatctgttgaaacagatttcttttttaacagatcttagtttaaaaaagtcataggtactgtgagttctgtataaactggtggacagtaagttagttcctttgttttaacttataagcctcaacttcaccgcagaataaagaatgtaggccaaagaaagcataatcggtcactcgtatagaacagtattgtttctataatttgaagctttctgaatggacgggttcaggcctgatgcaactgtaaaaagattacttaatgaatagactatatggaaattgtataaaatgttattaccttttatcgttagtagcttaaacagcactatatcactaattgctattcaaaatcaaaaacctgtttttgaatccccaagaaggcagcatgtgtatacaaccataccaccttgtacttaggggtgtgtcaaaacataattcaacagaaactttggctttaggaaagagtcacaaatatttaaaagaatggctttgtcattttaagtatacggtcggggaaaagtgtgctttaattaaatatacatcataccagtgatgctggcaaggttgaaagttactcctaatgttgatagctataaaaactagtttgtacataacaagacaatgagaggaaaaagcaatccctttgaaacaaaattctcaaataaaaaatgttcacagtggtttgtatcaacagtatgcattttatgacaagaacatgtacagattcagagcaccctagggctctctttatgtcctaaa

SEQ ID NO: 839 - DNA, 73 bp, Artificial Sequence
  Affymetrix ID 3432171
cttgccagcagtagaaaaaggaccgactataccgacctttctgattagtaaacagttgaatcaaggactctgg

SEQ ID NO: 840 - DNA, 55 bp, Artificial Sequence
  Affymetrix ID 3432352
gtcctacacgatggtgatcccaatgagcctgtttcagattacatcaatgcaaata

SEQ ID NO: 841 - DNA, 50 bp, Artificial Sequence
  Affymetrix ID 3435860
tgagctagcagtggcgatgacagctgtaaagcacgaacaggaatacatgg

SEQ ID NO: 842 - DNA, 779 bp, Artificial Sequence
  Affymetrix ID 3435864
ccgtcctaatggcagagctctctgacttgggtgtatgctgccaggctgggtactttcatactttgttttcttgttttgctttaaaactacgactcagcatacattttcccacatacatttttacattgtaccttaggactcagtcatctccacttaaattgatgacacaagcagctaataaccatttctgggtttctgcctaaccccctaattgtctgttaaagccaattctctgggtgtcccagtgagtggtggctttttttctttccacattggcacattcacttctcccactcttggcatgtaagaaataagcatttacataattggaaaaatctggatttctgatgccaaagggttaaagcttcttggatttcatttcattgatatacagccactattttatttttgatcagtggcctttgggccactgttcagggtactgaccatcagtgtcagcattagggttttggtttttgtttcttttgggtctttcttttttggcacatgtgaatcttgttttgtgtaaaatgaaattactttctcttgttctctgatgatgggtttaaaattaaaagagcatccggttttggtatggggatgatccaggattatgttgtgactgatacatattagttacttgtgcttttttttttttttttggatctttgcaagggcaaaactacaagtaacgagttttatataattaatttaaatttgttacaggttttcatgttcaggataaaccatacttccaccttgggtgagaacacttgcaa

SEQ ID NO: 843 - DNA, 80 bp, Artificial Sequence
  Affymetrix ID 3436017
actgcgtccggtccatctgacttctcaaagactttagaccttgacttcagtgatttgttgtagtcttgtatgcttctcta

SEQ ID NO: 844 - DNA, 29 bp, Artificial Sequence
  Affymetrix ID 3436264
tacagtatgtccaaatcaatttaataaaa

SEQ ID NO: 845 - DNA, 39 bp, Artificial Sequence
  Affymetrix ID 3438044
tccttgagtttcatatataagactgctgcagtcacatca

SEQ ID NO: 846 - DNA, 59 bp, Artificial Sequence
  Affymetrix ID 3445545
ggagatgacggactagaagactgtaaataagataccaaaggcactattttagctatgtt

SEQ ID NO: 847 - DNA, 32 bp, Artificial Sequence
  Affymetrix ID 3445674
tactgcacataaaaaagggtatttcatccaga

SEQ ID NO: 848 - DNA, 46 bp, Artificial Sequence
  Affymetrix ID 3445750
tcccttcattaacaggagaaatgcaaataccttcatatcccctcag

SEQ ID NO: 849 - DNA, 31 bp, Artificial Sequence
  Affymetrix ID 3446488
tgcccttttggcttacacactgggtgtgaaa

SEQ ID NO: 850 - DNA, 64 bp, Artificial Sequence
  Affymetrix ID 3446869
caatatgtgaatttgggctcacagaatcaaagcctatgcttggtttaatgcttgcaatctgagc

SEQ ID NO: 851 - DNA, 37 bp, Artificial Sequence
  Affymetrix ID 3446885
tattcctcagatcgtcaagtacagtcctgattgcatc

SEQ ID NO: 852 - DNA, 55 bp, Artificial Sequence
  Affymetrix ID 3452256
aacatctcctttttcgccatgtttgttatgtacttcttgactgccatttttggct

SEQ ID NO: 853 - DNA, 61 bp, Artificial Sequence
  Affymetrix ID 3452262
ttcaacaataagtgctaattcaacaaatgctgacacgtgtacgccaaaatatgttaccttc

SEQ ID NO: 854 - DNA, 112 bp, Artificial Sequence
  Affymetrix ID 3452324
cctgggtgtattaactttgtgtatatagtgtaaatatccccactgtactgttagaggccaacaattctagtatggcttgttggcaaagagtgctacaccgtttcaatgaaac

SEQ ID NO: 855 - DNA, 208 bp, Artificial Sequence
  Affymetrix ID 3452349
tgtatgtgcaggtactccaaagtccaaaacacttcggtcccaagcattttgaataagggatactcaacatgtgccagaaaatgtttgggcatgctatagaaaccattttcttctagcaaagttactcatcccagtgaactcacaggtatttatctaaagtaaacatggggaaggataatttacttcccaccctatgtgcagctcatta

SEQ ID NO: 856 - DNA, 457 bp, Artificial Sequence
  Affymetrix ID 3454729
tccacaagttaagtgcaccacagtgtagagagagacatacaacgctgaacttccataacagtcaatggtacagtcaaacatcacatgtacagaacacacaatttagatgaactgaaattataagataaaataaaataaaatccaatttcagaaaacaaaaatcaaaacattaaggatccctgaaatattcttaaaccctaatgagatttcactggactcaagtcattttgtagtgagacattcacaatatgaccctattaacccagtctaggaattctggggagccgaatgagtggccgcatcagacactctgacaaaaaatggtaaccagtttttgatctgaaaactcctcttaatttagctctgaacacatcaatcaagcttttgaaatgcatcctcctccttgcccctcatattttaaaccaactgttgttcacagtacagtttgcacgacatc

SEQ ID NO: 857 - DNA, 36 bp, Artificial Sequence
  Affymetrix ID 3456599
acttggaactttttgattgacaaattaataaacctc

SEQ ID NO: 858 - DNA, 80 bp, Artificial Sequence
  Affymetrix ID 3457550
cggcttcgggtcctcccaccacactctggttttctatgctgttttggtgcaagtacaactgtcgtagtcatggctttggg

SEQ ID NO: 859 - DNA, 90 bp, Artificial Sequence
  Affymetrix ID 3458494
tcagttcaagaagttccctcagatgacctcatatcaccggatgctattacaccgggtagctgcctattttgggatggaccacaatgttga

SEQ ID NO: 860 - DNA, 39 bp, Artificial Sequence
  Affymetrix ID 3461302
ccaaatttattgaactactgatgctaagttatacaaaat

SEQ ID NO: 861 - DNA, 86 bp, Artificial Sequence
  Affymetrix ID 3461431
tgtaacttcaattaagtcagctgtgtagtgagacaggttttgtgaatactcaaagaaaaatcagagctaaagcaatgaaagctagc

SEQ ID NO: 862 - DNA, 31 bp, Artificial Sequence
  Affymetrix ID 3462710
aacaagcagaagccatcagtaagagacaaga

SEQ ID NO: 863 - DNA, 30 bp, Artificial Sequence
  Affymetrix ID 3462846
cctagtcttgttacataaaggggatgtggt

SEQ ID NO: 864 - DNA, 31 bp, Artificial Sequence
  Affymetrix ID 3462861
gtaaacttttccaacgttggctgcagtaact

SEQ ID NO: 865 - DNA, 29 bp, Artificial Sequence
  Affymetrix ID 3462867
gatggaccagaaattatgggttgtacagg

SEQ ID NO: 866 - DNA, 25 bp, Artificial Sequence
  Affymetrix ID 3462868
tgctgacaaagacatacaggatgag

SEQ ID NO: 867 - DNA, 62 bp, Artificial Sequence
  Affymetrix ID 3462884
tcaagatttggatgatgttgaagaagtagaagaagaggaaactggtgaagaaacaaaactca

SEQ ID NO: 868 - DNA, 58 bp, Artificial Sequence
  Affymetrix ID 3462988
ctgaatctaaactttataatggctcagagaaggacagttcaacttcaagcaaactcac

SEQ ID NO: 869 - DNA, 50 bp, Artificial Sequence
  Affymetrix ID 3463596
agaacgacgacgaccaagagagaaaagaagatctacaggagtttcatttt

SEQ ID NO: 870 - DNA, 90 bp, Artificial Sequence
  Affymetrix ID 3463603
tcatacctcactcctgttagggatgaagagtctgaatcccaaagaaaagcaagatctagacaagcaagacaatctagaagatcaacacag

SEQ ID NO: 871 - DNA, 25 bp, Artificial Sequence
  Affymetrix ID 3464009
tttgtcgacataaactggattgcta

SEQ ID NO: 872 - DNA, 252 bp, Artificial Sequence
  Affymetrix ID 3465251
accttttactggttgcatggaaatcagccaagttttataatccttaaatcttaatgttcctcaaagcttggattaaatacatatggatgttactctcttgcaccaaattatcttgatacattcaaatttgtctggttaaaaaataggtggtagatattgaggccaagaatattgcaaaatacatgaagcttcatgcacttaaagaagtatttttagaataagaatttgcatacttacctagtgaaacttttc

SEQ ID NO: 873 - DNA, 131 bp, Artificial Sequence
  Affymetrix ID 3465252
gtgtcagttttcatagtatccatattttattactgtttattacttccatgaattttaaaatctgagggaaatgttttgtaaacatttattttttttaaagaaaagatgaaaggcaggcctatttcatcaca

SEQ ID NO: 874 - DNA, 96 bp, Artificial Sequence
  Affymetrix ID 3465278
cagttagctcatttgagataaagtcaaatgccaaacactagctctgtattaatccccatcattactggtaaagcctcatttgaatgtgtgaattca

SEQ ID NO: 875 - DNA, 49 bp, Artificial Sequence
  Affymetrix ID 3466861
tgacagagaatttggtggcattaaaagagatccgattggaacatgaaga

SEQ ID NO: 876 - DNA, 31 bp, Artificial Sequence
  Affymetrix ID 3467642
attgtaaaaagtccaagcagatcaataaact

SEQ ID NO: 877 - DNA, 31 bp, Artificial Sequence
  Affymetrix ID 3468015
gttagttgaaacattaaaaagcagacagtaa

SEQ ID NO: 878 - DNA, 33 bp, Artificial Sequence
  Affymetrix ID 3469369
ctttaaaggatctatttggactcgctagcaatg

SEQ ID NO: 879 - DNA, 74 bp, Artificial Sequence
  Affymetrix ID 3471177
ctgtgagaattgactggtcaactcttagtgtggtagatttgatggaagttaaccttatatcagcaggaactttg

SEQ ID NO: 880 - DNA, 261 bp, Artificial Sequence
  Affymetrix ID 3471377
tgcatcaggcctgaacccgtccattcagaaagcttcaaattatagaaacaatactgttctatacgagtgaccgattatgctttctttggcctacattctttattctgcggtgaagttgaggcttataagttaaaacaaaggaactaacttactgtccaccagtttatacagaactcacagtacctatgacttttttaaactaagatctgttaaaaaagaaatctgtttcaacagatgaccgtgtacaataccgtgtggtga

SEQ ID NO: 881 - DNA, 502 bp, Artificial Sequence
  Affymetrix ID 3471379
actggtcagcttgctcagatagatctgtgtttgtgggggcccttccttccatttttgatttagtgaatggcatttgctggttataacagcaaatgaaagactcttcactccaaaaagaaaagtgttttgttttttaattctctgttccttttgcaaacaattttaatgatggtgttaaagctgtacaccccaggacagtttatcctgtctgaggagtaagtgtacaattgatcttttttaattcagtacaacccataatcatgtaaatgctcattttctttaggacataaagagagccctagggtgctctgaatctgtacatgttcttgtcataaaatgcatactgttgatacaaaccactgtgaacattttttatttgagaattttgtttcaaagggattgctttttcctctcattgtcttgttatgtacaaactagtttttatagctatcaacattaggagtaactttcaaccttgccagcatcactggtatgatgtata

SEQ ID NO: 882 - DNA, 33 bp, Artificial Sequence
  Affymetrix ID 3472092
attgccacttcaaccaaaatcgatatcagcaat

SEQ ID NO: 883 - DNA, 94 bp, Artificial Sequence
  Affymetrix ID 3473482
ctgttataaacgttgaactttcacaatgttaaaatgttaaatatttggatatagcaacttcttttctcttcaaatgaatgccaagatttttttg

SEQ ID NO: 884 - DNA, 25 bp, Artificial Sequence
  Affymetrix ID 3474494
catgtgcagagtaacggtccaaacc

SEQ ID NO: 885 - DNA, 33 bp, Artificial Sequence
  Affymetrix ID 3474937
tctccctataaatgatgtatttgtgacacccta

SEQ ID NO: 886 - DNA, 87 bp, Artificial Sequence
  Affymetrix ID 3475623
ttacagctaaaacttacaaaggctaatgaaaatgcaagttttctgcaaaaaagtattgaggacatgactgtcaaagctgaacagagc

SEQ ID NO: 887 - DNA, 86 bp, Artificial Sequence
  Affymetrix ID 3475737
aaatcctctaagaaacataagtctgaggaacataatgacaaagaacattcttctgataaaggaagagagcgactaaattcatctga

SEQ ID NO: 888 - DNA, 31 bp, Artificial Sequence
  Affymetrix ID 3476294
ttttatagagaagcatacaagactacaacaa

SEQ ID NO: 889 - DNA, 70 bp, Artificial Sequence
  Affymetrix ID 3480098
agcattttggataatctgcttaggccaatgcatcagtatttttcttctgattggtacataacactaaaaa

SEQ ID NO: 890 - DNA, 149 bp, Artificial Sequence
  Affymetrix ID 3480100
caggaagcttgcactaagtaaggtaaattaacctaacaaatattttccaaagtggcatggtaaacatgccactactggcacatggcactttttgtgcagaacatggatatttttcctttaatatttagtacttgggctatcatgaaaca

SEQ ID NO: 891 - DNA, 34 bp, Artificial Sequence
  Affymetrix ID 3480173
acaactcagaaaggacctgaaaacttacattatg

SEQ ID NO: 892 - DNA, 91 bp, Artificial Sequence
  Affymetrix ID 3480201
ctagttctacagatagccctgtctggtatacgtctacttcactggaccgaaacaccttggaaaatatgcttgtacgggttcttctagtaaa

SEQ ID NO: 893 - DNA, 117 bp, Artificial Sequence
  Affymetrix ID 3480203
ctgaagccttacaaggttatgtagagagataccatcttctgtaccaaaaatagacaagagaatgctgtcaatattggtgtactgtaatgtgaatctatgctggtgaaaacaattttt

SEQ ID NO: 894 - DNA, 114 bp, Artificial Sequence
  Affymetrix ID 3480685
cctgagtcgtcacccttggattttatggatcacggagctgaccatctttacctggtcctggaactgaaaaactgtagcttgtgtgaaaatgagcctttggaccagtctttatta

SEQ ID NO: 895 - DNA, 95 bp, Artificial Sequence
  Affymetrix ID 3480856
ctaaaaggtgtaaatgaggagtgacatttctacattgtttattctgtacttttccttggagttaatgcaacacctcctgtcgcttagattttaaa

SEQ ID NO: 896 - DNA, 111 bp, Artificial Sequence
  Affymetrix ID 3481480
cagaattacagaagcgaacaatgaaatcagaaacaagctctcacagctgtagcatttataacaaagtcattatctacatgaccttgatttaagaggtgtagagtgtttctt

SEQ ID NO: 897 - DNA, 26 bp, Artificial Sequence
  Affymetrix ID 3482124
aagatgcacagaaagctgtagatgag

SEQ ID NO: 898 - DNA, 31 bp, Artificial Sequence
  Affymetrix ID 3482915
gcactgctttgtttaaaggactgcagaccaa

SEQ ID NO: 899 - DNA, 39 bp, Artificial Sequence
  Affymetrix ID 3482916
ctctcagagcatgcttttctttattaaaattactgatgc

SEQ ID NO: 900 - DNA, 29 bp, Artificial Sequence
  Affymetrix ID 3483215
gtttgcccagaaatctcagaaggctaaat

SEQ ID NO: 901 - DNA, 207 bp, Artificial Sequence
  Affymetrix ID 3484743
atttacactttaacaacttgctgggaatttaaacaacttgctgggaacatcctttgtgaggcactgtagcctttttttatttgaaactacttaaactccaagacaggctcactaactctttttcaagtgcaacaacaaatgtgttagctgaaaatagctaatttaatgattatttgtgacacagacacgattatgtgagccaagaga

SEQ ID NO: 902 - DNA, 146 bp, Artificial Sequence
  Affymetrix ID 3484750
gcattttgaggacttcgtttggatcccaattcaaacaaaataactgtgaagagattttttcgaacaacagaggagattcaattacacactgggttacatgatctgaaggaactggcatttttttaaatgtgtgataacggcactga

SEQ ID NO: 903 - DNA, 330 bp, Artificial Sequence
  Affymetrix ID 3484756
acgaatggtacttgttggtactacatacaactttcattcttatatatctatttttcaaataaatatacatattatatctaatcacctctaccatctcccactacaatgtctcctttaaattctatcgaatttccactactttattcacctttaatttcaacatactccaatatgttgtctcaaatctaacttttcttccttaggtttcttctatacccactcttagtcaagtggtcctaactagcagactcttttatccagtttccaaatttcagttatttattgtctctgatggtatatcccaaatctagaagacagcccatgtttgtg

SEQ ID NO: 904 - DNA, 319 bp, Artificial Sequence
  Affymetrix ID 3484762
tgctatatatctcatcgtctgcggatactaatggaggacgtgcttcctgtaaaacctgtgaatcaatggattcaataacatctggtctattaccaggcatctcatcatgcaaaggtctatgcaaatctgtagttttgattttgttctcctgcaaataactatgtcctctgacatcaatgatggtcacagggacccaatcatttccagttttctcttggattctgtgaaaatcagcattactatgattgtgaaaaacatacgactctgtggttgacttcaattttttacagcgtggctcactcgttacttcttctctagg

SEQ ID NO: 905 - DNA, 32 bp, Artificial Sequence
  Affymetrix ID 3485891
taacactgtgcacaaacgttttatactaaata

SEQ ID NO: 906 - DNA, 80 bp, Artificial Sequence
  Affymetrix ID 3489010
cagtcaagaaaaacattattgaggaaaattaatatcacagcataaccccaccctttacattttgtgcagtgattattttt

SEQ ID NO: 907 - DNA, 30 bp, Artificial Sequence
  Affymetrix ID 3489011
agtagcaaacagggctttactatcttttca

SEQ ID NO: 908 - DNA, 34 bp, Artificial Sequence
  Affymetrix ID 3494193
ttgaaagtgacacagattcggaatttacatttaa

SEQ ID NO: 909 - DNA, 58 bp, Artificial Sequence
  Affymetrix ID 3497656
ccaagaatctgcattgagaataactaaacattgttactgtacatactatcctgtttcc

SEQ ID NO: 910 - DNA, 120 bp, Artificial Sequence
  Affymetrix ID 3497658
tctgtaacacttaaagaattccctcattcattaccttacagtgtaaacaggagtctaatttgtatcaatactatgttttggttgtaatattcagttcactcacccaatgtacaaccaatg

SEQ ID NO: 911 - DNA, 143 bp, Artificial Sequence
  Affymetrix ID 3497823
ctgattcagaaaggcaccaagttagttttggaacaagttgtgacatccattgcatcagttgccgatactgcagaagaaaaatttgtcccctactatgatttatttatgccatcactgaagcacatcgttgagaatgcggttca

SEQ ID NO: 912 - DNA, 30 bp, Artificial Sequence
  Affymetrix ID 3498048
tccagcatattgatgttttaaggcaaaaca

SEQ ID NO: 913 - DNA, 92 bp, Artificial Sequence
  Affymetrix ID 3498445
ggtggaccgccatgagcatgaaaagacccgaagcaagttgactcttgcaatgtgcaactgttatgttctgcaaaatgagcaacgatgtatca

SEQ ID NO: 914 - DNA, 31 bp, Artificial Sequence
  Affymetrix ID 3498523
taataagaaggagacctgtaagcttgtttgt

SEQ ID NO: 915 - DNA, 32 bp, Artificial Sequence
  Affymetrix ID 3498537
ggtcattgttctcttcttatctggaatggtag

SEQ ID NO: 916 - DNA, 54 bp, Artificial Sequence
  Affymetrix ID 3498977
gtacatatgatttgtacttctgctgtgagattccctagtgtcaaaattaaatca

SEQ ID NO: 917 - DNA, 81 bp, Artificial Sequence
  Affymetrix ID 3499545
ttttgacatttgcatataaacatgcattagtaaataaaatgtatgggagaggccttaaatttgcaactaaacttgtggaag

SEQ ID NO: 918 - DNA, 338 bp, Artificial Sequence
  Affymetrix ID 3500408
cactcagggctgtaaagtaatttgccttgagtgtttcagtgactttcaccaagtttttggatcctggagtaatcagtgcaaacaagccataaagcttcagtagcaaattactgtctcacagaaagacattttcaacttctgctccagctgctgataaaacaaatcatgtgtttagcttgactccagacaaggacaacctgttccttcataactctctagagaaaaaaaggagttgttagtagatactaaaaaaagtggatgaataatctggatatttttcctaaaaagattccttgaaacacattaggaaaatggagggccttatgatcagaatgcta

SEQ ID NO: 919 - DNA, 1156 bp, Artificial Sequence
  Affymetrix ID 3501216
tgcgtctgtggcaatactagcaacaaattaataaaagcttaatttttatacaaatagttttattacaaatttgaattcttaagaaatacacatttaaagaaattacataaaaggccttagtagtcttagaaatgttttggagacttttagtgaaatgtcatttcaggcctagtggtccgaatctgccctcctgcggtccatgcgatgccctgctgaggtctgtgaacacagctcatgagaaaccacggaaatggcccgaatgtgcttacgtgtgaaaatactgatactgtgattcaacagagctgtttttcaagccaggatgcagaatgaggaatactaatgaaatgacggcctttaaggttgttgcttttgaagtcaagtcattcagtttgtgattagtgtttaaaaccctgaaaatatttaatacagaataaaaacaataagctcaaagtacatgtttcactataatagacaccatattcatgaacctgggtttggttttggcaacacataatttttggtttagaagtgaacaatgaaaacggatgtttcacattcaatatcctagtctttaaaaacctatgttaaaggacagcacagtctttcaaaggaagaaaactatgtaagctttattttaacagtggaagttaaactaaaccttgatctgcctaattgctgacatctatataaattactaatatatatatatatttaatttttgactatttttggaccatctttattccacaggaaaattgtgatttctgcaaaagcagctgcaaagtaccacacatagcagaaagacagaaatttatactggggggttggaagatatggctactgagtctgtaattccatttggaggttcaaaaaaccatttttacattgctattatttgtacagaccaagggacctaaattttgaaacagctagacagtgatataaacaaacatttatctctgggggtagaaaattaattataatacaagaatgaaaatgggcaaacagtatggaaggcacccacacctcctagcaccctttggttttctgatggagttctcacttcacacatcagtgcattggattgcagaaaatattgatattttatttcatcaaaagtgccatttggtatgccactattgaaagcttatcgctg

SEQ ID NO: 920 - DNA, 66 bp, Artificial Sequence
  Affymetrix ID 3501217
cagggtgtgttagttacgcaaattcctataaggcactttacggttttcatattggacagtgaggta

SEQ ID NO: 921 - DNA, 138 bp, Artificial Sequence
  Affymetrix ID 3501555
tctccataacggttgtacaagtctacattcccactaacagtgtttaaacgttcctttgcctctgcattctcgtcagcatttgttactgtcttttggtaactgtcattctaacgggggtaagacaatctctcattgtgg

SEQ ID NO: 922 - DNA, 93 bp, Artificial Sequence
  Affymetrix ID 3503211
ccgccttaagagactggatcgcacacctttgcaacagatgtgttctgattctctgaacctacaaaatagttatacatagtggaataaagaagg

SEQ ID NO: 923 - DNA, 31 bp, Artificial Sequence
  Affymetrix ID 3505453
tctgatttcattgttcgcttctgtaattctg

SEQ ID NO: 924 - DNA, 49 bp, Artificial Sequence
  Affymetrix ID 3506941
attttcttcttgtcaggatcatgtacaacagcatgcctagtgagacttt

SEQ ID NO: 925 - DNA, 601 bp, Artificial Sequence
  Affymetrix ID 3508731
tctcttgttgtaatactcttggctgttatggaagcacctgagtaatagagtggtgggtaggagctaggatgtttttctacaatcgaattttaaactaatttatctattttatagacactattgaacagttttttaatagttcatatctaaatctaacttttcataaaactttacggtttttccttcactaccttaaatatgcaagaaatactgacttggtatagggtaccttagttttctctattcattagacaggtaaaattatatttcagctgattgatctgtgtgacaaaattatttcttagctataatcagcacatcacttagttcaaacaaaattccccagcaaatgttagatagtaggtatatcagtcacctggggagttttcttcataatatgcatattcatcttgtaatgcatacatagttatcatcctccttctcaacccatctccctaaccccacatgcttgccagttcttgaagggataaagtgattctaataatgttttacttctctctgttcaatttaatgtgatataattctagtataaaaatattttggacagttgcttaacatggtcataagaggatttgtacta

SEQ ID NO: 926 - DNA, 108 bp, Artificial Sequence
  Affymetrix ID 3508732
acttgaaaaagagttagtgagcctgtcttggagtttaagtagtttcaaataaaaaaaggctacagtgcctcacaaaggatgttcccagcaagttgtttaaattcccag

SEQ ID NO: 927 - DNA, 30 bp, Artificial Sequence
  Affymetrix ID 3508761
gtaaaggtcaatgcgtaggggtagagtaag

SEQ ID NO: 928 - DNA, 103 bp, Artificial Sequence
  Affymetrix ID 3509912
atatatggaagtgacctaagaaataatagaaacatctttcagaagaatgtagtttgatatttatttagtataaaacgtttgtgcacagtgttaacaaatacaa

SEQ ID NO: 929 - DNA, 47 bp, Artificial Sequence
  Affymetrix ID 3509913
cagtattttacattgctagatgtacaaactttatacagaagcacaac

SEQ ID NO: 930 - DNA, 36 bp, Artificial Sequence
  Affymetrix ID 3510070
tctagaagacgattaagggaaggtcgttctcagtga

SEQ ID NO: 931 - DNA, 45 bp, Artificial Sequence
  Affymetrix ID 3510382
agaatgaagacctttcgaatccgtcaaaacctatacagaatcaaa

SEQ ID NO: 932 - DNA, 54 bp, Artificial Sequence
  Affymetrix ID 3512558
agggcgaaggtaccgaaagcacagtaatcactggtgtcgatattgtcatgaacc

SEQ ID NO: 933 - DNA, 189 bp, Artificial Sequence
  Affymetrix ID 3513710
caataactgtgcaatctgggaaagactgcatttaatttgttcagaaatttaccaagtccacactatttcagaaaattatataatcagtgacaatgttgtctgtggtatttgaatgactaattctatgtatggtatctgtccacttcattgtatcttcttgtgtagctaggaccaaacgtttatgaatta

SEQ ID NO: 934 - DNA, 104 bp, Artificial Sequence
  Affymetrix ID 3517677
gctcttttaagaaaatcgcaaatagtaaccatctatatatttgacaatatttggagtatttggggggcctttaaagtatgccacctggtttcatcttaaaatgg

SEQ ID NO: 935 - DNA, 135 bp, Artificial Sequence
  Affymetrix ID 3517683
acagctgtttgctccattatttgcataggaaatgaccacaatacaaaaataagagggaaaaagaagcaaaacagcaaccgatttctgcttttcatgtaggtgtgtttccacgtataaacattttgaagcctctta

SEQ ID NO: 936 - DNA, 79 bp, Artificial Sequence
  Affymetrix ID 3518501
ttttaatgaagtgcaatactgagtgtgcctcattatcttgcagctgtaaacatattggaatgtacatgtcaataaaacc

SEQ ID NO: 937 - DNA, 49 bp, Artificial Sequence
  Affymetrix ID 3519136
ggtgaaattgaagattgtcagattgatgattcctcacttcatgcagtaa

SEQ ID NO: 938 - DNA, 176 bp, Artificial Sequence
  Affymetrix ID 3521962
attagactcctgtttacactgtaaggtaatgaatgagggaattctttaagtgttacagaaagatttagtagaaatgttaccagtggtatggctgaaagaatatttcggtgaagtgctgttatatcctgaaaaccaagagtgaaatgtagttcccatacaagtggagagttagtctc

SEQ ID NO: 939 - DNA, 63 bp, Artificial Sequence
  Affymetrix ID 3524627
aggaagaacgtgcaaaacgtgaggagctagagcgaatactggaagagaataaccgaaaaattg

SEQ ID NO: 940 - DNA, 999 bp, Artificial Sequence
  Affymetrix ID 3524630
atggcttgtttgcactgattactccaggatccaaaaacttggtgaaagtcactgaaacactcaaggcaaattactttacagccctgagtgtctgtcaccatagtttgcataatgaatatgaatcccattggtgtgtgatgtaggaaatcctgtagttgtattttcttgaactgaaatatttgactcaaaataattaagactcattgtcatttttcatcttggcattattgtggacaagttgacatattaaatctctttgctttctggtaagcttagcttttaaaatgcattttcccttgtcctgtctttaactagatatacatgcttatatttatagtgggtttcacagactataaaattgaatgtatgaaatttttatttatatcagtgcttttaataatgaagatatttttggagtaatggtgctgtcttgtagcgagttattaatcatagtaagatttttttctcttcatttgctttttttgtttcatattaacaattttttttttacacggacacaaccctctgacagtctttccaaatattaaaatcatttgaatatgtatgctgtgatctgaacactgctcaagccatcaagcagtcttcatacagtttgcattataaaatctcattaaattctccaagaaaaaataagttgaagaattttatttcctgaccatgcatcccctggatttctgagtttcagttcagattgtagatgacaatataagctgccttccgaaattgtcaacatctgaatgttaagtccattttccccatggaagaagcccgtagttccatgaagtatggattaccatttgtatttttcactaacagtaaatgtatttttcttattaattgtttgccttaggaatgatgaattacattttttgttccttcttaccataaacatctgcattcctcagctcagccttccttgtatgttgtttctttataaatggttgagctgctgatgcaggtattgccaagctaacagta

SEQ ID NO: 941 - DNA, 57 bp, Artificial Sequence
  Affymetrix ID 3524631
agagttatgaaggaacaggttgtccttgtctggagtcaagctaaacacatgatttgt

SEQ ID NO: 942 - DNA, 69 bp, Artificial Sequence
  Affymetrix ID 3525314
ttgttgctagtattgccacagacgcattaaaagaaacttactgcacaagctgctaataaatttgtaagc

SEQ ID NO: 943 - DNA, 41 bp, Artificial Sequence
  Affymetrix ID 3525317
agatgtcagcaattaggcagatcaaggtttagtttaacttc

SEQ ID NO: 944 - DNA, 45 bp, Artificial Sequence
  Affymetrix ID 3525320
ctgactcagctaatgtcacaacatggtgctacttcttcttctttt

SEQ ID NO: 945 - DNA, 32 bp, Artificial Sequence
  Affymetrix ID 3525547
tcggggctgctaggtcagcattaaagtaaggc

SEQ ID NO: 946 - DNA, 51 bp, Artificial Sequence
  Affymetrix ID 3525702
gaacttcaacatggagttacttttgatccctcagttttaattcagtgtcta

SEQ ID NO: 947 - DNA, 54 bp, Artificial Sequence
  Affymetrix ID 3525704
tgctgttaagtacttctcccgtttgtttatcaacctgcagctaacaggatgtct

SEQ ID NO: 948 - DNA, 341 bp, Artificial Sequence
  Affymetrix ID 3525710
catttacgtgctcaactcctactgaattttagcaattaaaacatgaaaactgaaatattggttaacaaatgaaaatagaatttaaagacctgaacttcttttatgatctttaatgattatttgttctaatctttgacttggaaacccaaattattttcagtctaacgtacttgaaatttaactattaaaaaagttggaggaactcaaactttgaagttagggctccctttctccagataaagcacattgcctggtttattgtaaggcacttagaacatgtaaccaaaaacactcaatttaggaataggaatgattgttttacagctaagtgcgctctcaga

SEQ ID NO: 949 - DNA, 29 bp, Artificial Sequence
  Affymetrix ID 3526383
ttctttgtgtaaaaagcgtattctgaatt

SEQ ID NO: 950 - DNA, 37 bp, Artificial Sequence
  Affymetrix ID 3527530
gattttatttaatgtcataatgttgtcagaataaaga

SEQ ID NO: 951 - DNA, 58 bp, Artificial Sequence
  Affymetrix ID 3528208
ttgattattccataaaagtcaaatcattttgaacagcttcttttgaataaagacctaa

SEQ ID NO: 952 - DNA, 79 bp, Artificial Sequence
  Affymetrix ID 3529094
gtttgcgtatatagagttctcagacaaagagtcagtgaggacttccttggccttagatgagtccctatttagaggaagg

SEQ ID NO: 953 - DNA, 31 bp, Artificial Sequence
  Affymetrix ID 3531094
acagtgtcctaacagtgaaaatcagagttat

SEQ ID NO: 954 - DNA, 34 bp, Artificial Sequence
  Affymetrix ID 3531491
tgagaagcatatctggttacctttatgaatgtag

SEQ ID NO: 955 - DNA, 187 bp, Artificial Sequence
  Affymetrix ID 3533452
gtaatcagcacaatgatgtagaaatagaggaagcaggagaggaagaggaaaaggaaatagcgattgttcatagtgatgcagagaaagaacaggaggaggaagaacaaaaacaggaaatggaggttaagatggaggaggaaactgaggtaagggaaagtgagaagcagcaggatagtcagcctgaaga

SEQ ID NO: 956 - DNA, 129 bp, Artificial Sequence
  Affymetrix ID 3534950
tgcctatgatcaccttgcatggacagcaatcctgtaaacatcacagagtggcatcatttgtataattatatgcattgttgtagtttgcacctgttggttttaatgtgcatgtgaatggcctagagaacc

SEQ ID NO: 957 - DNA, 32 bp, Artificial Sequence
  Affymetrix ID 3535917
tgcatgacagatgttggcttattgtaaaaata

SEQ ID NO: 958 - DNA, 171 bp, Artificial Sequence
  Affymetrix ID 3536669
ttcatgtccgggtttcatcactgtgtagtcccatactgtttatcactccatcacaatttgaaggctgtctgaaatatccatgaactccactggtaattcatggagttcaactctcttaaccaccctcctccctgacccatgatttttaccctaacagccatgagagtttcc

SEQ ID NO: 959 - DNA, 67 bp, Artificial Sequence
  Affymetrix ID 3536745
accttacatgtgtaaaggtttcatgttcactgtgagtgaaaatttttacattcatcaatatccctct

SEQ ID NO: 960 - DNA, 119 bp, Artificial Sequence
  Affymetrix ID 3536931
gaagatgatcaagttgcacctgttccattgaatgtcgttgaaacttcaagtagtgttagggaaagaaaaaagaaggaaaagaaacaaaagcctgtgcttgaagagcaggtcatcaaaga

SEQ ID NO: 961 - DNA, 135 bp, Artificial Sequence
  Affymetrix ID 3536934
atattcctttgatggataatgctgactcaagtcctgtggtagataagagagaggttattgatttgcttaaacctgaccaagtagaagggatccagaaatctgggactaaaaaactgaagaccgaaactgacaaag

SEQ ID NO: 962 - DNA, 109 bp, Artificial Sequence
  Affymetrix ID 3536938
agggagaattgactacgcttatacatcagcttcaagaaaaggacaagttactcgctgctgtgaaggaagatgctgctgctacaaaggatcggtgtaagcagttaaccca

SEQ ID NO: 963 - DNA, 38 bp, Artificial Sequence
  Affymetrix ID 3536943
ttcagagctacatcaggaagagaacagcggaacatgag

SEQ ID NO: 964 - DNA, 38 bp, Artificial Sequence
  Affymetrix ID 3536948
agctgaagtgcagaaattacaggccctggcaaatgagc

SEQ ID NO: 965 - DNA, 32 bp, Artificial Sequence
  Affymetrix ID 3536949
gctgctgcacatgaattggagaagatgcaaca

SEQ ID NO: 966 - DNA, 31 bp, Artificial Sequence
  Affymetrix ID 3536951
gaagttcagaagctacagactcttgtttctg

SEQ ID NO: 967 - DNA, 30 bp, Artificial Sequence
  Affymetrix ID 3536992
gcacacattgttacagctagagtgtgaaaa

SEQ ID NO: 968 - DNA, 41 bp, Artificial Sequence
  Affymetrix ID 3536994
aatggaaagttaaggtcgatgaatcacacaagactattaaa

SEQ ID NO: 969 - DNA, 559 bp, Artificial Sequence
  Affymetrix ID 3537787
gaatccgtgttctttatgaggtatcttttatagttgtatctttttttttttctgagatggagtctcgctctactgtagcccaggatggagtgcagtagtgtgatcttggctcactgcaacccctgcctcccgggttcaaggaattctcctgccttagcctcctgagtagctgagattacaggcgcccaccaccacacctggctgatttttgtttcttagtagagacagggtttcaccatgttggccaggctagtctcgaactgacctcaagtgatccatccgccttggtctcccaaagtgttgggattacaggtgtgagccactgtgcccagccaagttatatctctaaagcaatgtgcaaaaataaactgaacttgggttgattaggtatattcaacatttgtcgggagagtagatgtttcattttatttcagtccctgtgtaatttgtcttctctaatgttaaatactatgtagaatgtgtctgtgtaattttatagatacttttattatggatggacattctaatttgtactgactttgggtctgtgaactacttc

SEQ ID NO: 970 - DNA, 32 bp, Artificial Sequence
  Affymetrix ID 3537795
ttctactccagtccaatgtaactatttagccc

SEQ ID NO: 971 - DNA, 34 bp, Artificial Sequence
  Affymetrix ID 3537796
ggattatacatactgtccaattttcattaaattt

SEQ ID NO: 972 - DNA, 1093 bp, Artificial Sequence
  Affymetrix ID 3539104
cactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaaca

SEQ ID NO: 973 - DNA, 131 bp, Artificial Sequence
  Affymetrix ID 3539877
cctccttgaagactgccgttatcagtatcgggaaccagcttcttcacctgaaagaaactgatacagctacactgagagcttctttagcacagtttgaacaaaaatggacaatgctcataactcaacttcca

SEQ ID NO: 974 - DNA, 36 bp, Artificial Sequence
  Affymetrix ID 3543929
aaattacatgatgcagagattcaggttttcctttaa

SEQ ID NO: 975 - DNA, 225 bp, Artificial Sequence
  Affymetrix ID 3544473
ctgcggctgtaaaagggcagtgcaagggatccatgtgacagaactgttctgtctcttgtgatggtggtcacatgaatctacacatgtgataatattgcatagaattaaatacacatacacgaaaaaagttcaagcagttgagcacaaatattttaattgtctaaaatgacattttctttaagagttatctacagttcaaagctcacttttatgaggtgtcacatc

SEQ ID NO: 976 - DNA, 29 bp, Artificial Sequence
  Affymetrix ID 3544890
gtaagcagctccgaatttttacctggaat

SEQ ID NO: 977 - DNA, 37 bp, Artificial Sequence
  Affymetrix ID 3545534
aggccaaaacttccgcaaacatctgatgatgaaaaga

SEQ ID NO: 978 - DNA, 105 bp, Artificial Sequence
  Affymetrix ID 3549043
gcaaatcacatgaactgtctaaccttcgactggagaatcagctgctgaggaatgaagttcagtctttaaatcaagaaatggcctcgttactccaaagatccaaag

SEQ ID NO: 979 - DNA, 107 bp, Artificial Sequence
  Affymetrix ID 3550451
gggtacaagactagacatgactgaaatggatttgggttttttggtgacctcccttactgggctaatcagcacttgatcggaagtccaggttagtatgtgaagccagg

SEQ ID NO: 980 - DNA, 1091 bp, Artificial Sequence
  Affymetrix ID 3550453
gtatgcacaaggtaggacttacttcgtaagaaacaaaatgccagtattttcttaagccatgatgtgaaaccaatgaccctgtgaccacatggcacagaacactaaattttggtcccatggctgaaacttgagggtgactaaaagtaatgcctgtgaaacatgatatctatctgggatggccatttgatctctaaaaggaattttgtacactccacagaactcctatctatagtaaaattgattttcagttttaaatgtgggcaaaaaggcattttctccaagattttaaaactaattcttatttttaaatggtttaccaaaatttgtcagtacattttacgtgtagaagcattttaaaaatcatttctagcaagcacttgacatctagtcagctctctactcctttattttgttttatcaaaagattaagagctcctttctttgaataaaataatttctcataattaagcagtagaagatctatcttcacaaagtatgagggatgccagatgttgataaacttactctttctgaatctggacaaagtcgacttaacagatttttctgatgagcatgttttatgaatcctccattgtgctccattctatcacatgtgcatttttcatgttaaactgcaattacttaatctcttcccctatccttctaaattaattttctgaagttggagtgtagtcttttcccccttaggctatgcattaatcgaagctttcttttcaccatgactttataatgtctagtaaacaatatttctacttcccacatctttgctttacacagtcaccttgcccttccttccaccaccgaagaaaaaagatggtcatactaacaggtgaaatgtacaaggtgtctgtgtgttttgtgtagcttcagagttagattgaaattaccaggcacagatttagtcttgtcattttgtttacacattggggaaaacaattcagtttattaaacgtttcatgtaactgcacccaagttttgccaagctggaaacttggaccttttctgtgtagtgactttttaattatagttttcataacctggagatcagactgttgctttcgcatgatgta

SEQ ID NO: 981 - DNA, 44 bp, Artificial Sequence
  Affymetrix ID 3550455
tatctgtactccttgtatttttcaagagctattttgtaaacaga

SEQ ID NO: 982 - DNA, 60 bp, Artificial Sequence
  Affymetrix ID 3552937
ttccgtacagatattgccaggacggaatacctttccaatgctgatgagcgtcttcgctgg

SEQ ID NO: 983 - DNA, 52 bp, Artificial Sequence
  Affymetrix ID 3552938
cctgctgatgacctttgcacagaaaatgccatcatgctgaaacgattcaata

SEQ ID NO: 984 - DNA, 45 bp, Artificial Sequence
  Affymetrix ID 3556198
tggaaatttggtgaagtgtgcaatctggtgtatatcatttttgaa

SEQ ID NO: 985 - DNA, 85 bp, Artificial Sequence
  Affymetrix ID 3556224
tgaaattttcttaacgctggagctatatgtgatctttagtgattatgaactaggtagaagaggatgaaatacgaactgctaagcc

SEQ ID NO: 986 - DNA, 87 bp, Artificial Sequence
  Affymetrix ID 3556278
cagcattgaagtcaaaaatttttctgtgatttacagggcatcccacgtgatttgttgtgtcccttcccttgctgttaatatactgtg

SEQ ID NO: 987 - DNA, 74 bp, Artificial Sequence
  Affymetrix ID 3556420
agctaagaatccatagccatggctctgtaagctaaacctgaagagtgatatttgtacaatagctttcttcttta

SEQ ID NO: 988 - DNA, 31 bp, Artificial Sequence
  Affymetrix ID 3558292
tctcctttacgctgtgtagagaatgctctgc

SEQ ID NO: 989 - DNA, 43 bp, Artificial Sequence
  Affymetrix ID 3559519
gtcagcctctactagaatgactgatcatgaaggtgcaagagca

SEQ ID NO: 990 - DNA, 52 bp, Artificial Sequence
  Affymetrix ID 3559556
aaaaaccaaaccagacgatcgatgctgtatttatgaaacgactgaagagatt

SEQ ID NO: 991 - DNA, 104 bp, Artificial Sequence
  Affymetrix ID 3562436
tactacactttggaccttctaagtagtacatatctgacaccaattaaaaatattgctcaaacaattgaatgcagagttcagcagagagctttctataggagaca

SEQ ID NO: 992 - DNA, 739 bp, Artificial Sequence
  Affymetrix ID 3563349
ataggttctctaggccattcacatgcacattaaaaccaacaggtgcaaactacaacaatgcatataattatacaaatgatgccactctgtgatgtttacaggattgctgtccatgcaaggtgatcataggcattatttatgaagccttaagatccagaagtgttgttactaccaaacctctgattaacactgtgaagtaagtgttttggaaggcagttccatgagttggctaacatttctttaaagcaaatgactgcttctaagcttagcctgaaagtggacaaggaaagagaaaagtaatttcactttactgtttggaagtacattcacttgaaaaactacataaaacatccctctttctttcttcaaagattttcataggaactggttatagcaatgaagttttattgcccttaaataatccataaacctagtttttcgagagtgctaagtcttaggaaacacaattttaaataatctaagtgccaagtacaaagttacttaccgtacaagagattttggttgaactgaaaatattagtatttcattactgtgtgcctgaaaaataaataggaagaacacatttaaaagcaaatgctttgcaagtaagtttcagtaataatgtgaaggtagtatacttacagctctgtgatggacaagcaagttgttggcacatccaccaaaaacaattacttctccttcatcgctggcacaagctgtgtgccataacctgcattta

SEQ ID NO: 993 - DNA, 38 bp, Artificial Sequence
  Affymetrix ID 3563970
tcaacattccataattttgtcaaaatagcactaaccaa

SEQ ID NO: 994 - DNA, 73 bp, Artificial Sequence
  Affymetrix ID 3566596
tgggccataatttgcaacacctgattcacagcataattttgtcacaaactgaaagtgttcctcaattaaagtg

SEQ ID NO: 995 - DNA, 464 bp, Artificial Sequence
  Affymetrix ID 3566603
ggacagtatgtataatccagggctaaatagttacattggactggagtagaaatttaaaatacaatagatgctggagtaaatgttacatattatcatcatctgattcatcttcttccttcagagattcctgttaagagaaaacaggtgtttaaaattgatttttaaaattgttatatcccacacctgtgggcaagttaagctacaacaacctgaattaagtggttaaattcctctacttgtaactgaatgaacagtttcagaataagatacccaagcaaagttaattaagcaaatgttgctctaacatgcaatgactgtatctttgatagaatatatacttaagaggcctggtgcagtgttcatgcctgtaatcccagcactttacgggggccaaagtaggcagatcacgtgagttcaagagtttgaaactagcctggtgaaaacccgtctctgtattattggtg

SEQ ID NO: 996 - DNA, 276 bp, Artificial Sequence
  Affymetrix ID 3566648
gtgatacatcgtaggcatccaaatgtttgccttgaatttttactgctatcacttggttgctactgacacccaaaccaatcagacctactaaatatcatatattattatctttatacctgtaattttcctataccatttccttttctctaaattatcagaattttcttcggtgctatcttaagaataattcccttatattatattaatcttgaatcccattgacttttcctgaaaaaaaattaactcccatttccacatggatcttaatagttcaac

SEQ ID NO: 997 - DNA, 45 bp, Artificial Sequence
  Affymetrix ID 3566657
tttaaagcaacttatgtgttcgatcatgtatgagatagaaaaatt

SEQ ID NO: 998 - DNA, 45 bp, Artificial Sequence
  Affymetrix ID 3566993
aataatccaaacaaatcttgaaccaatctataaacagaaaacctg

SEQ ID NO: 999 - DNA, 202 bp, Artificial Sequence
  Affymetrix ID 3569346
tggttcacagatagctgctttttattgacaaacaagatcatagtatttcagtcatggtgtcaagcacattaatacttttctgttactcagcatttttttttatatcagttcaaaagctacttttgccaagaaggatgagacaaaaggccagacagaaattggactttggttttcagacatgggactctcatgttaacaaaga

SEQ ID NO: 1000 - DNA, 299 bp, Artificial Sequence
  Affymetrix ID 3569757
gccatgctttcttccctgggttttaaacttcatataactttcagaaattggagagcaaaaattttgcttgtcactgcacatcaatataaaaaagcttatttaacttatcaaaacgtatttattgccaaactatgcttttttttgttaattttgttcatatttatcgggatgacaaatccatagaatatattcttttatgttaaattatgatcttcatattaatcttaaaattttgtgacgtgtctttttcctttttttccacagttttaatatattattcttcaacgacattttttgta

SEQ ID NO: 1001 - DNA, 242 bp, Artificial Sequence
  Affymetrix ID 3569759
cacaggccctacattaacaaggttaagctcaacccctttcccccagcacctcagaatgtgccctccctctccccctcataaccccacctaacataaggacaagtcaatttgtcagtagcttcttctggcttgaaaccccctccctggattttatagcccacttaccatgcataacagacaagtcccatattttgtcagtagatgcctttttttttccggcttaagccttaagtgccaaatca

SEQ ID NO: 1002 - DNA, 54 bp, Artificial Sequence
  Affymetrix ID 3569819
agaggaaagattaactatttgcaccgaaatgtcttgttttgttgcgacatagga

SEQ ID NO: 1003 - DNA, 127 bp, Artificial Sequence
  Affymetrix ID 3570456
cttggatatgggtaatttgatgacaaataatcttcactaaaggtcatgtacaggtttttatacttcccagctattccatctgtggatgaaagtaacaatgttggccacgtatattttacacctcgaa

SEQ ID NO: 1004 - DNA, 37 bp, Artificial Sequence
  Affymetrix ID 3573155
acgaaggtgactcactttgctttttcagtccattaaa

SEQ ID NO: 1005 - DNA, 47 bp, Artificial Sequence
  Affymetrix ID 3576551
ttcttggggaaatgacggtacattaaatcaaaattgaggatggatga

SEQ ID NO: 1006 - DNA, 28 bp, Artificial Sequence
  Affymetrix ID 3576567
aaagcactggaagatgtagattatgtac

SEQ ID NO: 1007 - DNA, 84 bp, Artificial Sequence
  Affymetrix ID 3576712
gtactattggcatttagaagactttaccattatttcagtgctaggcattattgattaggtcttggctccactgtttacctcttg

SEQ ID NO: 1008 - DNA, 161 bp, Artificial Sequence
  Affymetrix ID 3576730
gcagcagtcccaaatagtcaaaatgctactatctctgtacctccattgacttctgtttctgtaaagcctcagcttggctgtactgaggattatttgctttccaaattaccatctgatggcaaagaagtaccatttgtggtgcccaagtttaagttatctta

SEQ ID NO: 1009 - DNA, 29 bp, Artificial Sequence
  Affymetrix ID 3576939
actggctagattatcgaatgttcacattt

SEQ ID NO: 1010 - DNA, 32 bp, Artificial Sequence
  Affymetrix ID 3576940
ggaattgcaacccagtgaagaagttacctgga

SEQ ID NO: 1011 - DNA, 46 bp, Artificial Sequence
  Affymetrix ID 3577874
ggcagaatttaaagtttggttgataacaggatagataacagaataa

SEQ ID NO: 1012 - DNA, 99 bp, Artificial Sequence
  Affymetrix ID 3577898
gtctgaagctcgcataggcattcccagtacaaagtatacaaaagaaacaccctttgtttttaaattagaagattaccaagatgccgttatcattccaag

SEQ ID NO: 1013 - DNA, 83 bp, Artificial Sequence
  Affymetrix ID 3577911
ccagagctggcttatatcagtagcaatttcataactggacatggcattgggaagaatcagcctcgcaacaaacagatggaagc

SEQ ID NO: 1014 - DNA, 385 bp, Artificial Sequence
  Affymetrix ID 3578431
catgcgaaagcaacagtctgatctccaggttatgaaaactataattaaaaagtcactacacagaaaaggtccaagtttccagcttggcaaaacttgggtgcagttacatgaaacgtttaataaactgaattgttttccccaatgtgtaaacaaaatgacaagactaaatctgtgcctggtaatttcaatctaactctgaagctacacaaaacacacagacaccttgtacatttcacctgttagtatgaccatcttttttcttcggtggtggaaggaagggcaaggtgactgtgtaaagcaaagatgtgggaagtagaaatattgtttactagacattataaagtcatggtgaaaagaaagcttcgattaatgcatagcctaaggg

SEQ ID NO: 1015 - DNA, 34 bp, Artificial Sequence
  Affymetrix ID 3580188
ctttgtggaacgtcttcggaaacatggcttagaa

SEQ ID NO: 1016 - DNA, 118 bp, Artificial Sequence
  Affymetrix ID 3580204
gcattggacaaaatccggtatgaaagcttgacagatcccagtaaattagactctgggaaagagctgcatattaaccttataccgaacaaacaagatcgaactctcactattgtggata

SEQ ID NO: 1017 - DNA, 33 bp, Artificial Sequence
  Affymetrix ID 3580587
gaaagaaatgcagcttcatcgagaagactttga

SEQ ID NO: 1018 - DNA, 29 bp, Artificial Sequence
  Affymetrix ID 3580953
tataagtaccgtttacttcatggcatgaa

SEQ ID NO: 1019 - DNA, 56 bp, Artificial Sequence
  Affymetrix ID 3589660
ggttaaacaaactctcatttgactgtgagaactcatcgaaactggggaaccatgat

SEQ ID NO: 1020 - DNA, 34 bp, Artificial Sequence
  Affymetrix ID 3590159
ccctggcacttagtgggtctaataaagtatgttg

SEQ ID NO: 1021 - DNA, 45 bp, Artificial Sequence
  Affymetrix ID 3590352
cttccgtggattcatgcgaactttggctcatttccgccccattga

SEQ ID NO: 1022 - DNA, 41 bp, Artificial Sequence
  Affymetrix ID 3591851
ctacagaacaacatatcgtatcagatggcagacatacatca

SEQ ID NO: 1023 - DNA, 84 bp, Artificial Sequence
  Affymetrix ID 3591858
ttttcagtgtggacagcagatgaaggaattgagagcacagcatgaagaaaatattaaaaagttagcagaccagtttttagagga

SEQ ID NO: 1024 - DNA, 31 bp, Artificial Sequence
  Affymetrix ID 3591985
gatactgcggctgaaaagcactagtttgata

SEQ ID NO: 1025 - DNA, 65 bp, Artificial Sequence
  Affymetrix ID 3592046
gatttgaactcttcaatctcttgcactcaaagcttgttaagatagttaagcgtgcataagttaac

SEQ ID NO: 1026 - DNA, 158 bp, Artificial Sequence
  Affymetrix ID 3592047
tacatactctgcttagaatttgggggaaaatttagaaatataattgacaggattattggaaatttgttataatgaatgaaacattttgtcatataagattcatatttacttcttatacatttgataaagtaaggcatggttgtggttaatctggttta

SEQ ID NO: 1027 - DNA, 31 bp, Artificial Sequence
  Affymetrix ID 3592395
aaaacttgtgcctgataagctaaggctcatt

SEQ ID NO: 1028 - DNA, 30 bp, Artificial Sequence
  Affymetrix ID 3593171
tgtttttccgcaattgaaggttgtatgtaa

SEQ ID NO: 1029 - DNA, 561 bp, Artificial Sequence
  Affymetrix ID 3594101
gtaccatttcaattctgcattatagtagttcattgtataactgaaagaaatgatttcttcataagtgacattaaatatgaacattcatccaattgaatttacaaaatctttccaaaattataagggagaaaaatcgggtttacttcaatctttaaaaatctggcacctcttagtaactttcagtatttctaaattgctctaaaattttataataaatagattagggttttgcaatagtcttaatttttaagccaaaggttttctgagaccttaggttttgtagtctaaccagcttatgtggttatttaaaagaatattctttgtgtttttaaattgctatttttaaaaaagctttttattacccattttataaaatgttatactcatatttgtctgaatttttccagtattcctacatgaaattgtatgtattaaaaactcaatattagtggcaaatattaatactattaaaatggattttggtgctatattcttgtagctaaagcctagtagaattctcaagaataggtgaaatacactttcaaagttgcctgtcattta

SEQ ID NO: 1030 - DNA, 73 bp, Artificial Sequence
  Affymetrix ID 3595811
atgaaactgatgttacggattccggagaagtctgtggtctggtaaattgtatcaatcgctttaacatgactgg

SEQ ID NO: 1031 - DNA, 43 bp, Artificial Sequence
  Affymetrix ID 3595872
ccatcaagtggaagacaatctgggaatagtgaacgtaaacgga

SEQ ID NO: 1032 - DNA, 26 bp, Artificial Sequence
  Affymetrix ID 3596235
gaagatgatggaaatcatgatccgag

SEQ ID NO: 1033 - DNA, 37 bp, Artificial Sequence
  Affymetrix ID 3597406
gctggatcagactttactggagttaaacaacatgtga

SEQ ID NO: 1034 - DNA, 34 bp, Artificial Sequence
  Affymetrix ID 3597468
ctcttccaaggacggatgtagtaaatctctagcc

SEQ ID NO: 1035 - DNA, 107 bp, Artificial Sequence
  Affymetrix ID 3597671
atgaaatcgaggcatttgtaagttctcagaagtttttcttgcctctcaatatgaataaaacaaagcttttggattgctttcccatgtactagcagcccccaagtata

SEQ ID NO: 1036 - DNA, 32 bp, Artificial Sequence
  Affymetrix ID 3598717
ctctgcgatggatcttcttgctataaaaaagc

SEQ ID NO: 1037 - DNA, 90 bp, Artificial Sequence
  Affymetrix ID 3599449
tttgagtttgacatgtagagtcatttttagtttcatggcaattgacagtcctaataactcagctaatttgaaactaacaatcttgctgtg

SEQ ID NO: 1038 - DNA, 70 bp, Artificial Sequence
  Affymetrix ID 3602509
agatcactatgagatgaaggaagaagagcctattagtgggaaaaagtcagaggatgaaggaattgaaaaa

SEQ ID NO: 1039 - DNA, 737 bp, Artificial Sequence
  Affymetrix ID 3605249
cttgtcccttttatggctgcatgcagtttcaattgttcagtacaacagatgaggcatttaaaaggtctccaacgtcaagaaacactaactcatctctggcatatcatattttttaaggcagaagtattttctgtaatggttactacagagtgtttactggttaatttttagttaaccagaaccacacatcccatagataattccatttaactgaggtttatatccgtaagagcattaccatagaaaaatttccctttagcaatttcaagagacctcagccaccaatatacctaccttctttacaatataaagtgaaatattactttagatgaaaattttttgtatcttacttagaaaaaattaagttgatatttaaaagaattttgatttttaatcaccttccacaacgatttgatataccttaaactccactttcattttttataagagaatcactttcaagggaaaaaaatggatgttactatattttaaaatctgctttataaaaaagtgtataaatatcaatctgccagatatacttcctatccccaacacagctgtaacactgactaatggggtcatgaccatgaagcaaattttacttcctaaatagaaatgtgtaggtggcagaaagcgtatttttcagcaggagtgattctgttggatctctttacaatgtcagagcagttgttagaaatgttagtattttattcggtttcttgctgtgaaggattatc

SEQ ID NO: 1040 - DNA, 137 bp, Artificial Sequence
  Affymetrix ID 3606117
ggtctcaaaatgtttgctcgctttggaatctgtgaattcttacactgctccgagtcaacgctaagatcatggttacaaattatcgaagccaattatcattcctccaatccctaccacaattctacacattctgctga

SEQ ID NO: 1041 - DNA, 32 bp, Artificial Sequence
  Affymetrix ID 3606409
gaacaaaagtcagtcgtacattcagctacatc

SEQ ID NO: 1042 - DNA, 973 bp, Artificial Sequence
  Affymetrix ID 3608201
tcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcatttaaaaataaagttcctttatatttctgtcccatcaggaaaactgaaggatatggggaatcattggttatcttccattgtgtttttctttatggacaggagctaatggaagtgacagtcatgttcaaaggaagcatttctagaaaaaaggagataatgtttttaaatttcattatcaaacttgggcaattctgtttgtgtaactccccgactagtggatgggagagtcccattgctaaaattcagctactcagataaattcagaatgggtcaaggcacctgcctgtttttgttggtgcacagagattgacttgattcagagagacaattcactccatccctatggcagaggaatgggttagccctaatgtagaatgtcattgtttttaaaactgttttatatcttaagagtgccttattaaagtatagatgtatgtcttaaaatgtgggtgataggaattttaaagatttatataatgcatcaaaagccttagaataagaaaagctttttttaaattgctttatctgtatatctgaactcttgaaacttatagctaaaacactaggatttatctgcagtgttcagggagataattctgcctttaattgtctaaaacaaaaacaaaaccagccaacctatgttacacgtgaga

SEQ ID NO: 1043 - DNA, 28 bp, Artificial Sequence
  Affymetrix ID 3609166
gtatgacctcttgaaatgttaattttta

SEQ ID NO: 1044 - DNA, 75 bp, Artificial Sequence
  Affymetrix ID 3615597
ctaaaacctggcaaaacaagtgtcttcccggagatccaaattatctcgttggagcaaactgtgtttctgtcctta

SEQ ID NO: 1045 - DNA, 288 bp, Artificial Sequence
  Affymetrix ID 3615604
agtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcc

SEQ ID NO: 1046 - DNA, 43 bp, Artificial Sequence
  Affymetrix ID 3615659
aatgaccgagttgcaatggttaacggagtttcaatggataatg

SEQ ID NO: 1047 - DNA, 135 bp, Artificial Sequence
  Affymetrix ID 3615989
tttgtactaacagagggtgaaagctgttcacatttgtcaacaaaatctgcttgctgcagtagtaacctcaagtggttaaaacttgatttcccgagaaaactaaaacctttgtgcctaaaattgatgacttgagtt

SEQ ID NO: 1048 - DNA, 25 bp, Artificial Sequence
  Affymetrix ID 3621014
aagatgatggaaatcacgacccgag

SEQ ID NO: 1049 - DNA, 76 bp, Artificial Sequence
  Affymetrix ID 3623329
agttataacatgaagggattttcatcttttgctgtatgaaggataattgttatatcacatttggggggtaataaca

SEQ ID NO: 1050 - DNA, 425 bp, Artificial Sequence
  Affymetrix ID 3624199
ggttctttaaatcagtgggcagtcacttttgctgataagtctgcctttaccactgttctaactgtatctcacaaatttagatattgcggtcatcgatttcacctcaatgacctggcatgtcattcagttttaccactgttattgacatcctctcatcataatgctctattgactcctgaattagattgtcagtgggactcagacaataaattaagtagattaatggaccctgtaaaacatataaaaggttcctcgaaacaacctcttagaaatgcagcaactcgtacatttcatgacccaaatgcaatctacagtgaacttattctgtggcgtgtagacccaataggacctttgtcatacactggaggagtatcagaattggctcgaattaactctttacatacctcagcgttctctaatgtggc

SEQ ID NO: 1051 - DNA, 44 bp, Artificial Sequence
  Affymetrix ID 3624533
ctagaagaattgtctaatcagttacatcgcagtcaagaggagga

SEQ ID NO: 1052 - DNA, 32 bp, Artificial Sequence
  Affymetrix ID 3624702
gaccctagaataaaaggcttcaatactctgca

SEQ ID NO: 1053 - DNA, 32 bp, Artificial Sequence
  Affymetrix ID 3625565
tcacaattccagaacgactacttggacaaagc

SEQ ID NO: 1054 - DNA, 38 bp, Artificial Sequence
  Affymetrix ID 3626570
ccaaagagaaacctattaagtcatcatctccaaactaa

SEQ ID NO: 1055 - DNA, 45 bp, Artificial Sequence
  Affymetrix ID 3626585
tgtaaagatgaatgctgcttcgatgcaaatcaaccagagggaaga

SEQ ID NO: 1056 - DNA, 28 bp, Artificial Sequence
  Affymetrix ID 3626730
agaagagaaaagatcgtctgagaaatct

SEQ ID NO: 1057 - DNA, 166 bp, Artificial Sequence
  Affymetrix ID 3627045
tatgacttttttacaccatcttctgttattcattgcttttgaagagaagcatagaagagactttttatttattctagaattgcagaaatgactacactgtgctataccagagaattccagtagaaagaaacttgtaactctgtagcctcttacatcacctttatta

SEQ ID NO: 1058 - DNA, 76 bp, Artificial Sequence
  Affymetrix ID 3628011
tcaatgaatgtcagcgttaaacttaagacatgcacccttgatgatctcagagaaggaattgagagagcaacatcga

SEQ ID NO: 1059 - DNA, 128 bp, Artificial Sequence
  Affymetrix ID 3628052
tatgttgcccatattatgaccctgaagttagtaagcacctctgttacgataagagaaaacaagaatattccagaaatactaaaaattcagataattggcctgggcactcaagtatctcagcgaccagg

SEQ ID NO: 1060 - DNA, 81 bp, Artificial Sequence
  Affymetrix ID 3628057
cttgaagttcatataagaaggtatacacagatgtggtcatggagtaacataaaaaagcacaggcagttactcaagagttat

SEQ ID NO: 1061 - DNA, 74 bp, Artificial Sequence
  Affymetrix ID 3628471
ttctaattaatttaccaagataatgtaattacatttggttttgtaaggtatacagcagtaatctcctattttgg

SEQ ID NO: 1062 - DNA, 293 bp, Artificial Sequence
  Affymetrix ID 3628924
ccactggcctgtaattgtttgatatatttgtttaaactctttgtataatgtcagagactcatgtttaatacataggtgatttgtacctcagagtattttttaaaggattctttccaagcgagatttaattataaggtagtacctaatttgttcaatgtataacattctcaggatttgtaacacttaaatgatcagacagaataatattttctagttattatgtgcaagatgagttgctatttttctgatgctcattctgatacaactatttttcgtgtcaaatatctactgtg

SEQ ID NO: 1063 - DNA, 39 bp, Artificial Sequence
  Affymetrix ID 3630191
ttagatgaattgtacggcacttggcgtaaagccgcttcc

SEQ ID NO: 1064 - DNA, 28 bp, Artificial Sequence
  Affymetrix ID 3631414
cttaaacaaggtcagatgtatatggcag

SEQ ID NO: 1065 - DNA, 33 bp, Artificial Sequence
  Affymetrix ID 3632170
ctggcgctcgcattcaataaagagtaatgtggc

SEQ ID NO: 1066 - DNA, 28 bp, Artificial Sequence
  Affymetrix ID 3633110
gtaaagagtatcattcattgtttctcta

SEQ ID NO: 1067 - DNA, 34 bp, Artificial Sequence
  Affymetrix ID 3633223
gacattgctacttgagtgtgaacagttacctgga

SEQ ID NO: 1068 - DNA, 41 bp, Artificial Sequence
  Affymetrix ID 3633226
taaatgattttgctagtacagttcgtatcctagaggttgtt

SEQ ID NO: 1069 - DNA, 100 bp, Artificial Sequence
  Affymetrix ID 3634074
tgaatgagcccattcatggtaagtcttaagcttgttggaaataatgtacccatgtagactagcaaaatagtatgtagatgtgatctcagttgtaaataga

SEQ ID NO: 1070 - DNA, 79 bp, Artificial Sequence
  Affymetrix ID 3643958
gtgagtcagaatctatagccggttagtgatggtttcagacagaatcgtgttcgtgtctgttttgctcgattcttttcct

SEQ ID NO: 1071 - DNA, 33 bp, Artificial Sequence
  Affymetrix ID 3645267
gtcagaatctgagtccaagaaacgtaagcatag

SEQ ID NO: 1072 - DNA, 33 bp, Artificial Sequence
  Affymetrix ID 3645314
caacttttcatgtttcttaaaggcattttggtt

SEQ ID NO: 1073 - DNA, 34 bp, Artificial Sequence
  Affymetrix ID 3645591
agaaagaaggctaggcattaaggaacagcaagtg

SEQ ID NO: 1074 - DNA, 53 bp, Artificial Sequence
  Affymetrix ID 3647535
gccatccagcggcatctttccttgtcgaatgatactgtaatgaccttccaaag

SEQ ID NO: 1075 - DNA, 175 bp, Artificial Sequence
  Affymetrix ID 3648373
ttagctttaaccagcaaggatcatccaacctttaaagttactgatacaaagagtaaactgagtaacttgtagaagtggcacttgcaactgatacggttttcatactagaggcatgcagctctatttataaatactaccagaggaatttttgccaggtctcaaacatgctttattt

SEQ ID NO: 1076 - DNA, 163 bp, Artificial Sequence
  Affymetrix ID 3651472
tgaaaaaacaagctcgatttctggccgtaccaaaagcaaaatacaagtaaaaataacatttgaaaaccaagtttaacctcagagaatcatgttttaaaatacaaagtttaacattctttcctctatggagctaaacaagttttctaaaccataagactgcagt

SEQ ID NO: 1077 - DNA, 75 bp, Artificial Sequence
  Affymetrix ID 3653349
gagcacacatgtccgacgtgtcatcaaaatgatgtttctcctgatgctttaattgccaataaatttttacgacag

SEQ ID NO: 1078 - DNA, 32 bp, Artificial Sequence
  Affymetrix ID 3653673
tgttcctgttgacatgtcgttgtttaaataaa

SEQ ID NO: 1079 - DNA, 30 bp, Artificial Sequence
  Affymetrix ID 3655101
tgcgctgagctgcttatttattgaaaataa

SEQ ID NO: 1080 - DNA, 30 bp, Artificial Sequence
  Affymetrix ID 3658610
gtgcgaatgcagcatcaaggacatagctga

SEQ ID NO: 1081 - DNA, 44 bp, Artificial Sequence
  Affymetrix ID 3659201
ggaaaatgacttggtagttcatgtggcacttatagcagaaagcc

SEQ ID NO: 1082 - DNA, 136 bp, Artificial Sequence
  Affymetrix ID 3659319
cgccctattaggagaattacacatatctcaggtactttagaagatgaagatgaagatgaagataatgatgacattgtcatgctagagaaaaaaatacgaacatctagtatgccagagcaggcccataaagtctgtg

SEQ ID NO: 1083 - DNA, 98 bp, Artificial Sequence
  Affymetrix ID 3660917
caaactcattattacattgggtaagaaacaaaaaagaaagaatgagtcttcagatgaaatatctgatgcagaacagatgccacagcatacattaaaag

SEQ ID NO: 1084 - DNA, 116 bp, Artificial Sequence
  Affymetrix ID 3660927
gaccgtcctccttctaatatttggaagaaaatagatcaatccagggactataaaaatggcaatcaactcagggaatatcaactggaaggactcaactggctcttgttcaattggta

SEQ ID NO: 1085 - DNA, 33 bp, Artificial Sequence
  Affymetrix ID 3665635
ctgctgtacagctaataaatcataacggattaa

SEQ ID NO: 1086 - DNA, 78 bp, Artificial Sequence
  Affymetrix ID 3666111
taaaagcctctatgatccagagttgctatgcaccaaattttgatgccttctaaataccttgatgcctgtagcactaga

SEQ ID NO: 1087 - DNA, 118 bp, Artificial Sequence
  Affymetrix ID 3666869
tctctgggggaatttcatttgcatctatgtttttagctatctgtgataacttgttaaatattaaaaagatattttgcttctattggaacatttgtatactcgcaactatatttctgta

SEQ ID NO: 1088 - DNA, 34 bp, Artificial Sequence
  Affymetrix ID 3668992
tctgcttttaccaacaatttacttgaacttgtct

SEQ ID NO: 1089 - DNA, 43 bp, Artificial Sequence
  Affymetrix ID 3671113
aagccctcaaagaagtcggaatgtcatagtcttgaaatgaaag

SEQ ID NO: 1090 - DNA, 146 bp, Artificial Sequence
  Affymetrix ID 3677863
tggggtctccagctagtggaattcaaaacacaattggttctgttggcacagggcaacagaatgccacttctttaagtaacccaaatcccatagaccccagctccatgcagcgagcctatgctgctctcggactcccctacatgaac

SEQ ID NO: 1091 - DNA, 97 bp, Artificial Sequence
  Affymetrix ID 3679601
aaggtgtgaaattcctaacattgccaccagtgttacatctacaactgatgagatttatgtatgaccctcagacggaccaaaatatcaagatcaatga

SEQ ID NO: 1092 - DNA, 28 bp, Artificial Sequence
  Affymetrix ID 3683054
atttgcattaacatggcaaagattgtat

SEQ ID NO: 1093 - DNA, 117 bp, Artificial Sequence
  Affymetrix ID 3685080
gggttccatcgttccttttgtgatacagacgacctggaaacagtccatgaaagcgactgcatttttgcctttgagactcccgaaatatttaggcctgaaggaattctcagtcaaaga

SEQ ID NO: 1094 - DNA, 28 bp, Artificial Sequence
  Affymetrix ID 3687262
ttgaacctggcatatagtagctgcttaa

SEQ ID NO: 1095 - DNA, 65 bp, Artificial Sequence
  Affymetrix ID 3687792
tgccatctttgacacttgcaagctaaatgacattactcaaattaatcgttctgcacttcagcttc

SEQ ID NO: 1096 - DNA, 30 bp, Artificial Sequence
  Affymetrix ID 3690089
cctgcataagctgctacaaatagaataaag

SEQ ID NO: 1097 - DNA, 37 bp, Artificial Sequence
  Affymetrix ID 3692857
taatttgttttgctgagagcatagctatttgttttta

SEQ ID NO: 1098 - DNA, 40 bp, Artificial Sequence
  Affymetrix ID 3693546
gtaatgcggcattgatgcttgccaataaaaccaaccaacc

SEQ ID NO: 1099 - DNA, 108 bp, Artificial Sequence
  Affymetrix ID 3693711
cttgaagtttgaaatcgaggttctctgcaagaaccttgcattagacatcaatgagctaaaacctggaaacctcctaaaggataaagatcgcctgaagaatttagatga

SEQ ID NO: 1100 - DNA, 92 bp, Artificial Sequence
  Affymetrix ID 3693737
gcttccctatcaaacactgtccagacatgctggtattggccttactacaaattaacacctcttggcataccttgcgccatgaacttatctcc

SEQ ID NO: 1101 - DNA, 58 bp, Artificial Sequence
  Affymetrix ID 3693746
gccaaattttatctcgacgctgtcctatgccattgataatccattgcactatcagaag

SEQ ID NO: 1102 - DNA, 67 bp, Artificial Sequence
  Affymetrix ID 3696670
attaataaatgaggctagaatggcataaaaataaatggggattaaagaagtaattaggccggatgtg

SEQ ID NO: 1103 - DNA, 279 bp, Artificial Sequence
  Affymetrix ID 3697925
tccagtttgcaatctcgaaggtttgggtatgggaagggagaaagaaatacatttatattctcatactaaatatatataaatattcaagatccttaaaaatgcccaaaaggaccacatgtggtcaggattccagatttttaaagatctagagccaaaggctattctttattgaatcaagtgcttcagtgtttacaagggaaaactctaacgagactttcttactaccaatgaccatcttatctacaaaggttgttctgcctggtaataattggtggttta

SEQ ID NO: 1104 - DNA, 89 bp, Artificial Sequence
  Affymetrix ID 3699559
agacaagttcaagtaaattgttggtaaaagcagaagagctagagaaacctaaagaaacagaaaaagttaaaatcaccaaggtgtttgat

SEQ ID NO: 1105 - DNA, 45 bp, Artificial Sequence
  Affymetrix ID 3701588
tgattacgggatggttgtcacaattcacaaaaggtatgatacaaa

SEQ ID NO: 1106 - DNA, 39 bp, Artificial Sequence
  Affymetrix ID 3705531
aatataattccatttcgagtgattaaaacctatttgttg

SEQ ID NO: 1107 - DNA, 28 bp, Artificial Sequence
  Affymetrix ID 3707211
atcctgggattctagtatgcaataagag

SEQ ID NO: 1108 - DNA, 61 bp, Artificial Sequence
  Affymetrix ID 3708006
aggatggcaatcatgtcttgatgtcctgatttgttctagtatcaataaactgtatacttgc

SEQ ID NO: 1109 - DNA, 86 bp, Artificial Sequence
  Affymetrix ID 3709275
ctagtaaaatggagggacttaccatatgaccagtccacgtgggaggaagatgaaatgaatatccctgaatacgaagaacataagca

SEQ ID NO: 1110 - DNA, 144 bp, Artificial Sequence
  Affymetrix ID 3710735
tacatcagactctagaaaaaagggctgagaggaagcaagacgtaaagaattttcatcccgtatcacagtgtttttattgctcgcccagacaccatgtgcaataagattggtgttcgtttccatcatgtctgtatactcctgtca

SEQ ID NO: 1111 - DNA, 31 bp, Artificial Sequence
  Affymetrix ID 3714102
aaaatatgcaaacactctgtgatcaaactta

SEQ ID NO: 1112 - DNA, 1062 bp, Artificial Sequence
  Affymetrix ID 3715142
taagctgctatatgttgaatggacccttttgcttttctgatttttagttctgacatgtatatattgcttcagtagagccacaatatgtatctttgctgtaaagtgcaaggaaattttaaattctgggacactgagttagatggtaaatactgacttacgaaagttgaattgggtgaggcgggcaaatcacctgaggtcagcagtttgagactagcctggcaaacatgatgaaaccctgtctctactaaaaatacaaaaaaaaaaaaaattagccaggcgtggtggtgcacacctgtagtcctagctacttgggaggctgaggcaggagaattgcttgaacccaggaggtggaggttgcagtaagccaagatcacaccactgcactccaacctggacaacagagcgagactccatctcaaaaaaaaaaaaaaattgtgttgcctcatacgaaatgtatttggttttgttggagagtgtcagactgatctggaagtgaaacacagtttatgtacagggaaaaggattttattatccttaggaatgtcatccaagacgtagagcttgaatgtgacgttatttaaaaacaacaacaaagaaggcagagccaggatataactagaaaaaggatgtctttttttttttttttactccccctctaaacactgctgctgccttaattttagaaagcagcttactagtttacccttgtggtataaagtattataaattgttgtgaatttgaagaatccgtctactgtattattgctaaatattttgtttatactaagggacaattattttaagaccatggatttaaaaaaaaaaaaaaaaactctgtttctgcaggggatgatattggtgagttgccaaagaagcaatacagcatatctgcttttgccttctgttgtttatcttacctgcagatattaagaatgtatgcattatgtaaaatgctcaattatatatttttgttgagttttttaattaaagacttgtaaaaaaaaaaaacagtatattgcaaatgtttcttaaataggttaggtggagtactttctctctgtc

SEQ ID NO: 1113 - DNA, 57 bp, Artificial Sequence
  Affymetrix ID 3715434
cactttgtacaatgtagatttgaagtacagtggtgaaaacattaaatgtgacatttg

SEQ ID NO: 1114 - DNA, 674 bp, Artificial Sequence
  Affymetrix ID 3716021
atctaaccttttcacggagcttcggcaaaattcgagtgtgcaaaatgcatttctaaaacgtaatgcaagcaaagctttcacatttacactggcaggaaatacagagagaaactaacaagtcacattagggaaaattcagatcttttttttttttaatgacaagaattgcacagtttattattttgagacaattgttgcagacataaatatttaaaattttctaagcaaggtgcttttaacaaaatttttaaagttggaaagagctgataacttggatcatagctcacacagaattccaaattaaagtggactccattatctccctatattttgcaaacaatgctttgtataacacttctttaaaacactaaaagagacagcaagctgaaacttttttcaaagcacacaagaaatgtttacttgaagaaggtgctgaggggagaagggagtgaagaatccttttactatttcccactacaggacagccactaacagactaagaacaaaaaagatacaacaaaaaaagtaaatcacttccccagcttataaaatagttccagtttcgcaagatctgtaactttaaacagagagcaccagttgggcaatagaatgagcatttcatactgcataaaaattagtgaggtggtaagaaccatcgactatctcaggcatta

SEQ ID NO: 1115 - DNA, 33 bp, Artificial Sequence
  Affymetrix ID 3716674
tatagatttctttgaactcggaatctcatagca

SEQ ID NO: 1116 - DNA, 1047 bp, Artificial Sequence
  Affymetrix ID 3716757
ctcctgtgaacaggcagattctataggtataaattaagatgtcatttccctagagaggctaagaaataaatacattcaacaaatatttgatgaattaaaggaatgagatgaagatatctggagaaaacgactcattcgatccaccattcattctgcacacattgtgtcaggcaagatgctcagtatcagacacagaatataggaaagtacaagtaaccagctgggtgtggtcactcatgcctataatcccagcactttggaaggccaaggctggggaggactgcttgaggccaggagtttgagaccagtctggacaacacagcaagaccctcattctctacaaaaaatatttttaaaaaagtttttaaaagaaagtacaagaaggtacacataccctttcacaagtcacagtgatatcttactaaaatgcctggagccacaagaagcctgttttcccttcaaatgaaaagtaggttaaaactggattcaccaaatctcacagaagaaaaaagtacacacactgaacttttttagttttttattttgattcctatggctggacaaggtatctacattctagaacatcttcttaatccctaaacaagtggaaatctactacttatatataaaaagacaagtagaaaaggtcatgattgtctaacagggaacatgaatccccaaataaattatcttacaaaaatatcatacttgcttttctacttttttactatactggtaaaaatgaaatgaaaaaaataattcacattaaaataaaatctgtagggtggcatagaagacaattatttttacatattaaagtccttcaaataaatattttacacacaaatttaagtaagtttttaaaaaaaataagtatttacgctatatttttttgctctgctaaaaagagaccacttattaaactgacattaaactggactgcagctcattctctgcaaagagtaaaatgcatgtcacaggcagattggatacacacgtgcatattacatacatatgaaatggctaacactgctgtgcatactactg

SEQ ID NO: 1117 - DNA, 37 bp, Artificial Sequence
  Affymetrix ID 3717647
agagaaaatacacccagtcattttgcggtgaaaacat

SEQ ID NO: 1118 - DNA, 1167 bp, Artificial Sequence
  Affymetrix ID 3717681
ggacttcagtttgcgcttcccaataactttaaatatatccaccttttccaagtgtttaagccctaaaacatctatctttatttttttacctggcatttttgttagaaaatagattacgttttaaagtaacattaaaaaaattcttcagtaagataattgcttttaataagtatatttttcttgctcttggctctatacatttttgcataatattttagtcttgaaaagcttttcattttcttaactctggaaaataatagtttggaactaaattagtttgagaaatctgaatcatctctctttaaatctgttcagttagtctcatgtattctcaggggaaaatgtctttattttactaagcggaagtagtagtcatattgaggcagaaatttgtagcccaaacaaggtaggtcttcgctgaaactaatttgaactattcctatttgaagcaaattgaacactattataaatctagactggctaattagatggaaactttgatggttaggttctttaagaggcatacaatccgttatgagaatgtttatgtttatgttttaatttttatcaaaataacctcagaggaggtacatgtgagtaaactgtcttaatggttgattacttggaacccacccattaattctaaatgatgtaatggtctataaggtagtaatctggcacagcttcagtaatgtgtgctgtgtttgtaaaaaggtgaagtactttattggcattttctaagccctctgacattgttagtgtctgggttggttggtttgcattttactattatggtggaatctgtgttccttcaccattggttaagtctgaggaactaacagtgaaattcattgatggccaacttggataaaaatgaagaatgatattttcccagtttactacacatggaccaatagtgattatttgggagagaacactgggtagtattgtttgacttaagaaccttaaatatattgaaaagtaatggcattcttgctcatgatttgcatttgttagtgaggtctcaccatagtattttaatgtagcttcttattaaaattttattcataaatagtttagcagtcacagaggtgaaaaaaaaaagatttgcaccatcatatccgagtctttactaataaaatgaggctgcttaagtacccaagtgga

SEQ ID NO: 1119 - DNA, 266 bp, Artificial Sequence
  Affymetrix ID 3717684
acctgagtatgtgatgtggcaatgcattcttctagataagcactaaacaaagtatggaccctcaatttatgtctttaagatttaaagtgaagtaaatttctaaggaactgtgtcctttcctagcaggaataaacagtgaaaatttggtaagtatttaacttgaagtgcatgtaatagtgatgagagtaagtagccaaatttccgtaatataaggtaatgtttaaaagtgagcaataattattgcatctcttttgcgacttcatgta

SEQ ID NO: 1120 - DNA, 33 bp, Artificial Sequence
  Affymetrix ID 3718837
cccaacccttggagctaaatgcgttgtaaaata

SEQ ID NO: 1121 - DNA, 28 bp, Artificial Sequence
  Affymetrix ID 3719123
cacttcatatggagttaaacttggtcag

SEQ ID NO: 1122 - DNA, 67 bp, Artificial Sequence
  Affymetrix ID 3720778
gttgtctgtgttgtaattcataacttttgataccatttctgatgtgtaaaattggttgtcttgtaaa

SEQ ID NO: 1123 - DNA, 58 bp, Artificial Sequence
  Affymetrix ID 3720990
ttgttttaggccttctagttccacaccatcttcttgagggcttccttcagtattttca

SEQ ID NO: 1124 - DNA, 99 bp, Artificial Sequence
  Affymetrix ID 3722990
atgcactcaaataaacagacagatgaagcagtatgctatttccaaaagcagtgcaagtggagtgaaagcatttccatacgaacctggctttaaaaactg

SEQ ID NO: 1125 - DNA, 30 bp, Artificial Sequence
  Affymetrix ID 3723305
ctcgtgattggcatataatggagttaacgg

SEQ ID NO: 1126 - DNA, 74 bp, Artificial Sequence
  Affymetrix ID 3726811
gcttgctcagtgtttaattgcaagttttcaatcttggactttgaaaacaggattaaacgttagtattcgtgtga

SEQ ID NO: 1127 - DNA, 116 bp, Artificial Sequence
  Affymetrix ID 3728316
cggttggggaaagatacttatgatgaaggatattttttaatttaacttttttttaaatattggtaataggtcggcaacagcaactatagaagtacaactcaatagatggcattaaa

SEQ ID NO: 1128 - DNA, 134 bp, Artificial Sequence
  Affymetrix ID 3729194
gtagatgtctttatggaatacaatctaattcagcagtgtactgcattcttgcttgatgctctgaagaataatcgcccatctgaaggtcctttacagacgcggttacttgagatgaaccttatgcatgcgcctca

SEQ ID NO: 1129 - DNA, 304 bp, Artificial Sequence
  Affymetrix ID 3729227
gcgctgatcctgtagtcacctattttcgtactgaaacatcgtctttacccacttctcagtttataatgggggaaaacaggcaacgtgttcttgtaacctttatttcatgaaggacttctttttgtttctaactataaacttggatcacctatgttaaaaccttatttcacattccacatcattttagaatttattttcgaaggggaatagtttcaatgttttattcacttgggctttttttcttccccctctttctttaaagaactgctcaatattcaatctgttgtgaagaacctgatttgca

SEQ ID NO: 1130 - DNA, 96 bp, Artificial Sequence
  Affymetrix ID 3732475
atagagcctgattctgaaaaagatgaggtaaaaggttcagatgctgcaaaaggagcagaccaaaatgaaatggatatctcaaagattactgagaag

SEQ ID NO: 1131 - DNA, 256 bp, Artificial Sequence
  Affymetrix ID 3732477
aaggagctcttagattctgacagtgataaaccctgcaaggaagaaccaatggaagtagacgatgacatgaaaacagagtcacatgtaaattgtcaggagagttctcaagtagatgtggtcaatgttagtgagggttttcatctaaggactagttacaaaaagaaaacaaaatcatccaaactagatggacttcttgaaaggagaattaaacagtttacactggaagaaaaacagcgactcgaaaaaatcaagttgg

SEQ ID NO: 1132 - DNA, 31 bp, Artificial Sequence
  Affymetrix ID 3732479
gtaaaaaaccaagtcagcagaagaaattaga

SEQ ID NO: 1133 - DNA, 48 bp, Artificial Sequence
  Affymetrix ID 3732480
gagagaccagttaataaatgtagtgatcaaataaagctaaaaaatacc

SEQ ID NO: 1134 - DNA, 39 bp, Artificial Sequence
  Affymetrix ID 3734754
gaactgcttcaatgtggtatctttgtatggcaatgtata

SEQ ID NO: 1135 - DNA, 28 bp, Artificial Sequence
  Affymetrix ID 3734863
ctgggagaagtttctactggtgtatatt

SEQ ID NO: 1136 - DNA, 122 bp, Artificial Sequence
  Affymetrix ID 3737304
tggaaataattcagtccaaacagtcttccaagggacccttgctgctacgaaaaggtggctccgagaagtttttacaaagaacatgctcacatcttcaggtgcctcattcacatacgtcaagg

SEQ ID NO: 1137 - DNA, 106 bp, Artificial Sequence
  Affymetrix ID 3737985
cttattccagtttcgtgaggctagcatgaggtgtgtgcatttgccaggggcaaatttctattctcaattaacccatgcagcaaatgctacgcatctgctgagtccg

SEQ ID NO: 1138 - DNA, 69 bp, Artificial Sequence
  Affymetrix ID 3738300
tgcgtttgttttgccatcactatgttgacacttttatccaataagtgaaaactcattaaactactcaaa

SEQ ID NO: 1139 - DNA, 59 bp, Artificial Sequence
  Affymetrix ID 3739125
cactgttgtagtaaagagacatatttcatgaatggcattgatgctaataaatcctttgc

SEQ ID NO: 1140 - DNA, 31 bp, Artificial Sequence
  Affymetrix ID 3739523
ccctgtggcacgttatgcttcagaattaaaa

SEQ ID NO: 1141 - DNA, 69 bp, Artificial Sequence
  Affymetrix ID 3740129
actggagccacagatacagaatctgtattgttcttactgaaacacagcatggaattaacattaaactta

SEQ ID NO: 1142 - DNA, 26 bp, Artificial Sequence
  Affymetrix ID 3740130
ttgaagctaatttgtactactggata

SEQ ID NO: 1143 - DNA, 34 bp, Artificial Sequence
  Affymetrix ID 3744423
atacaaggaagaaaccattgagaagatgcaggaa

SEQ ID NO: 1144 - DNA, 30 bp, Artificial Sequence
  Affymetrix ID 3746953
aactttggactaattgcatcatacttggag

SEQ ID NO: 1145 - DNA, 53 bp, Artificial Sequence
  Affymetrix ID 3746967
ccactgtataaccagccatcagataccaaggtgtaccatgagaacatcaagac

SEQ ID NO: 1146 - DNA, 31 bp, Artificial Sequence
  Affymetrix ID 3747223
gggcactgcgaatgccatgactgaagaatta

SEQ ID NO: 1147 - DNA, 67 bp, Artificial Sequence
  Affymetrix ID 3748953
ggcaatgacttttaagtttgatcacagagtgtttgcatattttcttactatttttggtatgatttaa

SEQ ID NO: 1148 - DNA, 30 bp, Artificial Sequence
  Affymetrix ID 3750786
tagagccaactgagataaatgctatttaaa

SEQ ID NO: 1149 - DNA, 29 bp, Artificial Sequence
  Affymetrix ID 3752711
gccatcaagagcaataaagcattaaatct

SEQ ID NO: 1150 - DNA, 124 bp, Artificial Sequence
  Affymetrix ID 3754530
ggaccactgcatggaatgttaatcaatactccatatgtgaccaaagacctgctgcaatcaaagaggttccaggcacaatccttagggacaacatacatatatgatatcccagagatgtttcggc

SEQ ID NO: 1151 - DNA, 116 bp, Artificial Sequence
  Affymetrix ID 3754741
ttttatgatcccagcatgaatgttattttcatggaatacttgaagtcttacagtcacctgtaccaaacatttgaaatcaactacaagtacatgggactggtgataaatgatcctaa

SEQ ID NO: 1152 - DNA, 127 bp, Artificial Sequence
  Affymetrix ID 3756204
aacaagatgaacaagtcggacttcctgggaaaggggggaaggccaaggggaaaaaaacacaaatggctgaagttttgccttctccgcgtggtcaaagagtcattccacgaataaccatagaaatgaa

SEQ ID NO: 1153 - DNA, 78 bp, Artificial Sequence
  Affymetrix ID 3759590
ctggcctggccgtaggtttgtaactgtttcatagaagagccctggagaagacagtagaatgagcctatctagtttaaa

SEQ ID NO: 1154 - DNA, 40 bp, Artificial Sequence
  Affymetrix ID 3760197
atgctagatttaattgtaagcagctggtttaagaaattat

SEQ ID NO: 1155 - DNA, 417 bp, Artificial Sequence
  Affymetrix ID 3761752
ccacatttgttcgaagtatccttttctagtggttagaactgtccctataatttgattgtccctcatatactaaccgaaagcaaaatgctctgatgagatgcttataaactatgaattttttaattcttgaaatttttttcagtttttatgattgttgaatagatatgtatgtttagttccattattatttcactggaagttttaaattttgagaagatgaagcagagatagaaatccattgcagatatatgtatatttaccccatcttgcaggtttcagttagtaaaacacagaatgataggtcggttttaatgttttaaaaaaaaactaaaaaactaaagttagagaaacctttctgtttgagctactgtatagaaaagacaacttttattgcattgctggtccacatgataaata

SEQ ID NO: 1156 - DNA, 75 bp, Artificial Sequence
  Affymetrix ID 3762575
tgaaggttacagatgcgccaaataaatcagagataagcaaacacattgaagtacaggtagcccaggaaactagaa

SEQ ID NO: 1157 - DNA, 173 bp, Artificial Sequence
  Affymetrix ID 3764121
cgctggagcaatctccttggaaggattcagcacgagtagatggtaaacatttaaaggggaaagggggggtttgtttaaaatagtaaatcagtaagtcacttctaaatttaaagaaaacaaaattggagttgaagaataagtaggtttccaattggctattgccgttttctttg

SEQ ID NO: 1158 - DNA, 69 bp, Artificial Sequence
  Affymetrix ID 3764125
acatatctgaagagatggattaagaatgctttggattaaggattgtggagcacatttcaatcattttag

SEQ ID NO: 1159 - DNA, 108 bp, Artificial Sequence
  Affymetrix ID 3764127
gatgattggtgacactttttgtagaacccatgttgtatacagttttcctttattcagtacaatcttttcattttttaattcaaactgttttgttcagaatgggctaaa

SEQ ID NO: 1160 - DNA, 90 bp, Artificial Sequence
  Affymetrix ID 3764884
acaacccctccatcacaagtgtttgaagcctgtcagattctaacaacaaaagctgaatttcttcacccaacttaaatgttcttgagatga

SEQ ID NO: 1161 - DNA, 282 bp, Artificial Sequence
  Affymetrix ID 3764921
aggatcatctataatgcttgaagtgtgtaacttacaaaacctaaagaattaaaaatgaaaagtaagcaaccccaactactctgaaatctccattactctcctttttacacccaactatcaagatttcactttgttggatgtaaaatcagtgttgaatcacatagtttagaatgggatgagtgtgaggttagctcttctactaggacctaagaataaactctttgattaaactcaactctgaagtcgccatgcagatactcatgagtattgtcaagacagtta

SEQ ID NO: 1162 - DNA, 131 bp, Artificial Sequence
  Affymetrix ID 3765738
tctgatgaattagtgcagcaatatcaaattaaaaaccagtgtctttcagcaatagcatctgatgcagaacaagaacctaaaattgatccatatgcatttgttgaaggagatgaggaattcctttttcctga

SEQ ID NO: 1163 - DNA, 77 bp, Artificial Sequence
  Affymetrix ID 3765761
tgtaccagtgtggaaattaaccaacatcaacctgtataccttctcagtgaagagcatatcacccttgctcaacagtc

SEQ ID NO: 1164 - DNA, 117 bp, Artificial Sequence
  Affymetrix ID 3765767
ggactttcctatgaatgccgtactctgcttttcaaagcagttcacaatctattggaacggtgtttaatgaacaggaattttgtacgtattggcaagtggtttgtaaagccttatgaa

SEQ ID NO: 1165 - DNA, 275 bp, Artificial Sequence
  Affymetrix ID 3766896
cagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatc

SEQ ID NO: 1166 - DNA, 56 bp, Artificial Sequence
  Affymetrix ID 3768039
gtgatacaaataattccggacctgaaattaataagattcgaacaccagagaaaaag

SEQ ID NO: 1167 - DNA, 101 bp, Artificial Sequence
  Affymetrix ID 3768121
aaagagcgagtggaatttattttggagcaaatgaggctctgcctagctgtgaaggattacattcgaacacaaatcatcagcaagaaaattaacaccaaatt

SEQ ID NO: 1168 - DNA, 429 bp, Artificial Sequence
  Affymetrix ID 3768258
tccttctaacgactttctgtaattcacattagtattgcctggcaatttaggaacaaacctataaacatgagttttactaatccagctccaaccaccatatcctgtcactctgtactcttcacctttttgtttccaaacctggtgtaaatgaaaacattttgattaaacaataagaataacaatgcttataaaccacatgtggttttatgcatttttaccaaacaagtttcaaagtcaggcaaatgaaatttactaatcatccccaggtttttccaagtggggacaaaaaaaatttactaatcaatttagttttcactgggaatcaaagattaatgaactaatactcctttttagcatgtaaaatgtaacacaattttacagttttataacaatttcaacaaattggccttgtaagcctccaatattcta

SEQ ID NO: 1169 - DNA, 51 bp, Artificial Sequence
  Affymetrix ID 3769780
tggacaagacctcataactgtgattaatatcaataaaaaggggatgttgtg

SEQ ID NO: 1170 - DNA, 25 bp, Artificial Sequence
  Affymetrix ID 3770244
gaccagggtgtaaaggtatctgctg

SEQ ID NO: 1171 - DNA, 27 bp, Artificial Sequence
  Affymetrix ID 3770564
gaagctctggcccttgtattacacatt

SEQ ID NO: 1172 - DNA, 98 bp, Artificial Sequence
  Affymetrix ID 3770565
cagatgaccattgaggacttgaatgaagctttcccagaaaccaaattagacaagaaaaagtatccctattggcctcaccaaccaattgagaatttata

SEQ ID NO: 1173 - DNA, 47 bp, Artificial Sequence
  Affymetrix ID 3770746
ctctgcggatagcatttggtaggtagtgattaactgtgaataataaa

SEQ ID NO: 1174 - DNA, 57 bp, Artificial Sequence
  Affymetrix ID 3771039
ccctggatgcgctaacattcactcttgtttgtccctggactggccatgaagtgagga

SEQ ID NO: 1175 - DNA, 62 bp, Artificial Sequence
  Affymetrix ID 3771094
ctgctttttgtaacttgaaaaactggacatctatgttatatgtactcatggataaatatttc

SEQ ID NO: 1176 - DNA, 30 bp, Artificial Sequence
  Affymetrix ID 3771337
agattcatagatgagctttcattgtaaaaa

SEQ ID NO: 1177 - DNA, 88 bp, Artificial Sequence
  Affymetrix ID 3776189
gctttactgctgtggttatcacaggggtacaaccagaacacatacagtacttgaaaaattatttccacctttggacacgacagttagc

SEQ ID NO: 1178 - DNA, 115 bp, Artificial Sequence
  Affymetrix ID 3776219
ccaaaccttccagtctatgttgtagattgcagtagttctggaaccagtattttaacaggatctgcaattcaagttcagaatattaaaaaagaccagacgcttaaagcaagaattg

SEQ ID NO: 1179 - DNA, 70 bp, Artificial Sequence
  Affymetrix ID 3776236
taaagtggggacatattgtatccagtttggttttatgatggataaaacaaatattctcaacagtgaacag

SEQ ID NO: 1180 - DNA, 35 bp, Artificial Sequence
  Affymetrix ID 3776446
caataactgtggtctatacagagtcaatatatttt

SEQ ID NO: 1181 - DNA, 44 bp, Artificial Sequence
  Affymetrix ID 3776461
cttccagttacattgtcttactctcttttacttctcagacactt

SEQ ID NO: 1182 - DNA, 77 bp, Artificial Sequence
  Affymetrix ID 3778266
ccacaccagttagcattctttttggttatccactctctgagcgaaaacagatggcacttcttatgcagatgacagca

SEQ ID NO: 1183 - DNA, 80 bp, Artificial Sequence
  Affymetrix ID 3778629
caaactgttcggctactggacaggttgtatattaccagatcatcactagcagatgtcagttgcacattgagtcctttatg

SEQ ID NO: 1184 - DNA, 188 bp, Artificial Sequence
  Affymetrix ID 3778630
tagattgctaatcccactcattcaggaaatgccaagaggtattccttggggaaatggtgcctcttacagtgtaaatttttcctcctttacctttgctaatatcatggcagaatttttcttatcccttgtgaggcagttgttgactgagtttttcatccttacaatcctgtcccatggtatttaacata

SEQ ID NO: 1185 - DNA, 102 bp, Artificial Sequence
  Affymetrix ID 3779850
cagctaagtgaaggatcaattacacttcaggttgaagcagtagagagtacttcacaagtggatgaaaatgatgtgacgttaacggctgataaaggcaaaaca

SEQ ID NO: 1186 - DNA, 46 bp, Artificial Sequence
  Affymetrix ID 3781686
ttgctgaaggaccatttattactggagaaaacattgatacttccag

SEQ ID NO: 1187 - DNA, 42 bp, Artificial Sequence
  Affymetrix ID 3781689
aggttgactggcaggatactcgtgatgatccctacagaccag

SEQ ID NO: 1188 - DNA, 74 bp, Artificial Sequence
  Affymetrix ID 3783364
ggtggaccgctctgtatgactctcatgcttcaaaactattttttattcaagtgacttacaatggccctaggaaa

SEQ ID NO: 1189 - DNA, 62 bp, Artificial Sequence
  Affymetrix ID 3787934
ataatcatattatctaaggcagaagtggtggttacttggcaagaatacgatttcttttaatg

SEQ ID NO: 1190 - DNA, 669 bp, Artificial Sequence
  Affymetrix ID 3789817
gaagccattcagtaagcctgctgtgagttaataaggtagcacagattagatctaaggacatggtgggggcaactcaaacagccactcacaattgttattaagattccagctacaatttcttttggaacttatttattcttaattacttaggaattaccaggaaagagggaaaatcaatctgcctctttaccttaaattaatgctaagagggatactgtgaatcactaaaaaactctccccaaaattatgttcttacttcaaatcctttaaggcaattctatttttacaaattcttcaatttctatgcctcaggattattagcagctaagcagagttagaaaagattaataactgatacaaaaggctattcatttgattctagtgtttactgctgaattatactgtgttctaatatgagcagatttttatacgatattgccaatttcttcccccaagatttttgttttaattcattgatgcaataaaataaatcaaagaattcaaggtacacttaaaaggacagaaagataagttactggagtatcttacaccataatctaaaattgttgaattatacatacatataacttgaataaaatgaataacttggataaagtgagtaaaatgctgaaatgtatccctaactacagtttcatgccaggtattttc

SEQ ID NO: 1191 - DNA, 43 bp, Artificial Sequence
  Affymetrix ID 3795442
atgctgtgattctttcggactggtagattatcatggagtatct

SEQ ID NO: 1192 - DNA, 28 bp, Artificial Sequence
  Affymetrix ID 3796554
acatagcaaacacattggatgttgcacg

SEQ ID NO: 1193 - DNA, 292 bp, Artificial Sequence
  Affymetrix ID 3798470
tttacactgtaagaggcaccatttccccaaggaatacctcttggcatttcctgaatgagtgggattagcaatctaaataaatcatatttcaagaggtaacagcaacagataaaatttaaagggattattaaaataacatttacaagactctgaacaattcttgaactcttattaaaaccacaaagaaagaacaattctttatttatgaatttcataaaggactcaatgtgcaactgacatctgctagtgatgatctggtaatatacaacctgtccagtagccgaacagtttg

SEQ ID NO: 1194 - DNA, 25 bp, Artificial Sequence
  Affymetrix ID 3800623
aatgtgtatcagataagaagcactg

SEQ ID NO: 1195 - DNA, 52 bp, Artificial Sequence
  Affymetrix ID 3804170
ttggacatgggcgtatcttctaacgatgtcgtcaccattagtatattcttcc

SEQ ID NO: 1196 - DNA, 183 bp, Artificial Sequence
  Affymetrix ID 3804202
agtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtatta

SEQ ID NO: 1197 - DNA, 53 bp, Artificial Sequence
  Affymetrix ID 3806255
gtcattgttctagtaaattagttccatttgtaaaagggttactctcatactcc

SEQ ID NO: 1198 - DNA, 53 bp, Artificial Sequence
  Affymetrix ID 3806256
ctcagaacaatcagatgcaaagctgaaagagattgtaacaaatttcttggctg

SEQ ID NO: 1199 - DNA, 80 bp, Artificial Sequence
  Affymetrix ID 3807476
gcatcttccttgtaattgtctctgacctttttatctgagaccggaattcaggataggagtctagatatttacctgatact

SEQ ID NO: 1200 - DNA, 92 bp, Artificial Sequence
  Affymetrix ID 3807570
ttaagtttgatcaagccatggtgacacaaaaatgcattgatcatgaataggagcccatgctagaagtacattctctcagatttgaaccagtg

SEQ ID NO: 1201 - DNA, 48 bp, Artificial Sequence
  Affymetrix ID 3807629
ttggagcagagatacgacaaccttcgggatgaaatgaccatcataaag

SEQ ID NO: 1202 - DNA, 35 bp, Artificial Sequence
  Affymetrix ID 3808602
atactttattgaaaggggacacctgtacattcttc

SEQ ID NO: 1203 - DNA, 39 bp, Artificial Sequence
  Affymetrix ID 3809343
gtgtttccattgtaatcttgacacatgcaattgtaaata

SEQ ID NO: 1204 - DNA, 129 bp, Artificial Sequence
  Affymetrix ID 3809673
accatgtccttagatctaatctgtgctaccttattaactcacagcaggcttactgaatggcttcatttcagatttagttgatttctccaccaaatgcatgtcatgtattctcaataggctgtattccca

SEQ ID NO: 1205 - DNA, 127 bp, Artificial Sequence
  Affymetrix ID 3809834
ggcttggatacttcttattggacttttgtgaatgctttttcaatttttggaagcattgcactttattttggcatcatgtttgactttcatagtgctggaatacatgttctctttccatctgcatttc

SEQ ID NO: 1206 - DNA, 189 bp, Artificial Sequence
  Affymetrix ID 3813298
ctcctcagaagtcagcgcaggaagagcctgctttggacacgggagaaaagaagccattgctaactacttcaactgacagaaaccttcacttgaaaacaatgattttaatatatctctttctttttcttccgacattagaaacaaaacaaaaagaactgtcctttctgcgctcaaatttttcgagtgtgc

SEQ ID NO: 1207 - DNA, 43 bp, Artificial Sequence
  Affymetrix ID 3813299
ggccgtcgccttgatgtatcgcctatacatggcagaggactga

SEQ ID NO: 1208 - DNA, 34 bp, Artificial Sequence
  Affymetrix ID 3815669
acgtacgcatactccatcgatgttgtatttgcag

SEQ ID NO: 1209 - DNA, 32 bp, Artificial Sequence
  Affymetrix ID 3816402
tgctggtttaagatgattcagattatccttgt

SEQ ID NO: 1210 - DNA, 32 bp, Artificial Sequence
  Affymetrix ID 3820658
ctgcattccaaccactaataaagtgcctattg

SEQ ID NO: 1211 - DNA, 155 bp, Artificial Sequence
  Affymetrix ID 3820705
tttaaaactttgattgcatgatgtattttttttttagaaagttattgtttgagaataatgtctttttataccaggaaaatagttatcctgaatgacgttgaaaactccccctcccctttatttttttttaatcaatacatgtgaaagtaacaagc

SEQ ID NO: 1212 - DNA, 29 bp, Artificial Sequence
  Affymetrix ID 3820721
catcccagaataaatggagacttcatgtg

SEQ ID NO: 1213 - DNA, 32 bp, Artificial Sequence
  Affymetrix ID 3820752
ctggaagatactgaaggattcctttttgaaag

SEQ ID NO: 1214 - DNA, 43 bp, Artificial Sequence
  Affymetrix ID 3822055
cttcaactacaagggcaagaacgtgctgatcaacaaggacatc

SEQ ID NO: 1215 - DNA, 35 bp, Artificial Sequence
  Affymetrix ID 3825428
taagttcttttgtcaccaaatattaataaacagtt

SEQ ID NO: 1216 - DNA, 29 bp, Artificial Sequence
  Affymetrix ID 3829039
aaagtaatggactctgatgaagatgacga

SEQ ID NO: 1217 - DNA, 32 bp, Artificial Sequence
  Affymetrix ID 3829810
actcaaataacgactatacttatggaccaaat

SEQ ID NO: 1218 - DNA, 39 bp, Artificial Sequence
  Affymetrix ID 3831128
tctgaggacttaggtattttaagcgaataaactgacagc

SEQ ID NO: 1219 - DNA, 55 bp, Artificial Sequence
  Affymetrix ID 3831280
ctaaaattttatcacggatgaatggatggacttgctctctatgtaatatgaaatt

SEQ ID NO: 1220 - DNA, 31 bp, Artificial Sequence
  Affymetrix ID 3835412
cattgtcatggactccttaataaacgtgaag

SEQ ID NO: 1221 - DNA, 30 bp, Artificial Sequence
  Affymetrix ID 3837493
gttcaaacgcaagtacaaggtgaagctggt

SEQ ID NO: 1222 - DNA, 73 bp, Artificial Sequence
  Affymetrix ID 3838612
gctattgagggcacctacattgacaagaaatgccccttcactggtaatgtgtccattcgagggcggatcctct

SEQ ID NO: 1223 - DNA, 35 bp, Artificial Sequence
  Affymetrix ID 3838790
gctggccatcaagccatactatcagaagaaggaca

SEQ ID NO: 1224 - DNA, 44 bp, Artificial Sequence
  Affymetrix ID 3840344
gtgaaattgctaatcaacttgagaagtctaccagtgatgcttcc

SEQ ID NO: 1225 - DNA, 83 bp, Artificial Sequence
  Affymetrix ID 3841271
gggaccaaatcaagacatgggtagcgtccaacgagatcaaggacaagaggcagcttatagacaaccgcaagctcattgagacg

SEQ ID NO: 1226 - DNA, 25 bp, Artificial Sequence
  Affymetrix ID 3843668
cgcactcggcttatccctagaaatc

SEQ ID NO: 1227 - DNA, 35 bp, Artificial Sequence
  Affymetrix ID 3845710
ttgtgtcaagatgatccaataaagttgtaaaacag

SEQ ID NO: 1228 - DNA, 181 bp, Artificial Sequence
  Affymetrix ID 3846115
tctttttccagtatactagcttgtcttttaagaaaggggatattaaaaaaaaaaaaagacaaaagtgtttttaaaaaaaagcaacacccacacctgtgtctgtatatagtcagcttatctcgtgttcaatcgtctgatctctacagagagaagtggaaaatgctgtatcaagggtgggctt

SEQ ID NO: 1229 - DNA, 224 bp, Artificial Sequence
  Affymetrix ID 3846291
atccgacgggaacatctccaacagaagcaaaacggaaagtgcctcccggacccccagagggccacccaacctcaccagtcaccagccccagaccacccacagcccctcccagacaccccgcctcatctggaaatagttccgtttgtttctctaaaaagacttgtaggtgggaaaaaaaatcttttgttctcatggaattggcctattggcaagatcgcatgttt

SEQ ID NO: 1230 - DNA, 68 bp, Artificial Sequence
  Affymetrix ID 3846561
gtgtttgatgcgatcatgaatttcaagaaagaggagacagcaaaactgatagagaaactggacatcaa

SEQ ID NO: 1231 - DNA, 38 bp, Artificial Sequence
  Affymetrix ID 3846784
ctgcacatagacctgcagttagtaaatcataagcccaa

SEQ ID NO: 1232 - DNA, 30 bp, Artificial Sequence
  Affymetrix ID 3847357
gagtgtggcatagaagctatttaatgatta

SEQ ID NO: 1233 - DNA, 39 bp, Artificial Sequence
  Affymetrix ID 3849774
ttaacaatactcttcggtacacacaaagtatgtgaaagg

SEQ ID NO: 1234 - DNA, 333 bp, Artificial Sequence
  Affymetrix ID 3849776
tgccctggcttttaggtcttctctatggaggatatggcaaagacatacaaaattatatatacgtatccacacattaataactctatttctatattaaaaacatgaccacaagccaacacttttaattccactgcagcactgctgggttatttcttgctttccaaattcccttgtctgtagctatctttcatcacttgcaaaaatagcacgtgctcaatcctagattaaaatataagctactctcagaattgataactcataccactctttttaaaaatcaactaaccagaattcagtttttattcacagttcttatctttagctcagggcgta

SEQ ID NO: 1235 - DNA, 259 bp, Artificial Sequence
  Affymetrix ID 3850502
gactgtttgccaatggttgtatttagtaagatttgtagactctgtttttcttttgacacagctgcaaggccaacagctgtgcaaagccacaagttatgctaagtcagcagttatgctatagattacatgacctgtgactgtatcattaactgcttttgttttgcttctgtaagtttgcctataaaaaccacactcagtctttgttcaatggtcagcttttcagatacaaatccactgagccggtgtacatctaaataaa

SEQ ID NO: 1236 - DNA, 113 bp, Artificial Sequence
  Affymetrix ID 3851604
gctgcggtcccaggaaggtcttaccccctcttctatttattaatttgcagacccagcccctcccctactttttggtcagctacgtctctagaataagatggtatctgaagtcc

SEQ ID NO: 1237 - DNA, 63 bp, Artificial Sequence
  Affymetrix ID 3851902
accttgcaacggatgtccttgttgatcagcacgttcttgcccttgtagttgaagatgacatga

SEQ ID NO: 1238 - DNA, 65 bp, Artificial Sequence
  Affymetrix ID 3854371
cttttggatgtgataagctttgtaattgtcttttaatgagctctcatcttggagagatacattct

SEQ ID NO: 1239 - DNA, 82 bp, Artificial Sequence
  Affymetrix ID 3855101
aacagcatctgtaaacatcgggtacaaaaatattccattaaactctttcccgaggtggctccgtttgctccatggaggcctt

SEQ ID NO: 1240 - DNA, 322 bp, Artificial Sequence
  Affymetrix ID 3856046
tatcaaatattacgggagctcaatagaaatcaacaatatgaatctttatttaccacaaacattattgatgcctatgcttgtttttcttaaaatctattagctttttatgacacatttatactttttcagttgtttattactgagaagtggctgtcctgccagaaaactgctattctcagctgtatccacaatgactaatagtgagtggaagtgcagtggataaaagcaaatgtgtcttctctgtattttttttcatccattggctaaaaaacaggaggatgcagaagatgaaagaaaatacaatccctgaaagatcatgaag

SEQ ID NO: 1241 - DNA, 114 bp, Artificial Sequence
  Affymetrix ID 3857120
gtgggaaatatttgaaagtcttctataaatttttaaattcaaacagacatacgataagacatactggaaagaaatgcttcaaatgtaaaaaatgtgtcaagtcattttgcatcc

SEQ ID NO: 1242 - DNA, 67 bp, Artificial Sequence
  Affymetrix ID 3857884
gtagaactcagactccttggaaagaaaaaaaagaggctccgggttgacaaatggtcgggtaacagaa

SEQ ID NO: 1243 - DNA, 67 bp, Artificial Sequence
  Affymetrix ID 3862168
agttcagcgctgtcaggattgcgagagatgtgtgttgatactgttgcacgtgtgtttttctattaaa

SEQ ID NO: 1244 - DNA, 65 bp, Artificial Sequence
  Affymetrix ID 3862473
tgagcatgtgtggttatcctgtagagcttttcccaagaagggtgtttgaacttagagtcttaata

SEQ ID NO: 1245 - DNA, 108 bp, Artificial Sequence
  Affymetrix ID 3867099
cgcacgctctatctcttcaactggatctggcgctaccatttcgagggcttcttcgacctcatcgccattgtggcaggcctggtccagacagtcctctactgcgatttc

SEQ ID NO: 1246 - DNA, 49 bp, Artificial Sequence
  Affymetrix ID 3869659
tgttcagtcgcaattcctaccttgtacaacatctgatcattcatactgg

SEQ ID NO: 1247 - DNA, 30 bp, Artificial Sequence
  Affymetrix ID 3869971
cccattacattcaggtctaaggctctttat

SEQ ID NO: 1248 - DNA, 184 bp, Artificial Sequence
  Affymetrix ID 3870593
gagacctcctgagacttaaaccctgtagaaaacagatgaatcaaacccaggccccattctcgggaagctcacagtgtgaaaataggtaactacaaaaacacagccaataagtgctttgaaacaaggaagaacataaaaaagatgtagtgttcgaaaacctggaatcaaaaactaaccgcggaga

SEQ ID NO: 1249 - DNA, 33 bp, Artificial Sequence
  Affymetrix ID 3872197
tgaatctatgctctcaggataatcaactcaaaa

SEQ ID NO: 1250 - DNA, 192 bp, Artificial Sequence
  Affymetrix ID 3872208
ctattcttcaggatacactgctaagataacaaaaagaaaccaaatacagataaatggtttgcaaaacatgtatctgacaaataactagcattcagaatattaaaagatacaatgaattcttaaaattcaatgttaagaagaaaaaacaatttaaagtgggacaaaagatttggacagatagggtctggtgca

SEQ ID NO: 1251 - DNA, 39 bp, Artificial Sequence
  Affymetrix ID 3874114
tcctctctgctagtgatcgtgtttattatcatagttttg

SEQ ID NO: 1252 - DNA, 75 bp, Artificial Sequence
  Affymetrix ID 3874280
cgccttccatggtttttaaatgcagtaaataacatttctggatgagacttgtttccaaaataaaccagctatatc

SEQ ID NO: 1253 - DNA, 47 bp, Artificial Sequence
  Affymetrix ID 3875278
tggagactctttcttttgttaagcagttaaaggaataaaagagctgg

SEQ ID NO: 1254 - DNA, 28 bp, Artificial Sequence
  Affymetrix ID 3878051
ttgatgtgaaattaaattcttattggcc

SEQ ID NO: 1255 - DNA, 101 bp, Artificial Sequence
  Affymetrix ID 3878053
tggggagaggtaccatgatgacactcacagggagccactgttcactgacacttggaagcggtcattgttaatatcacgggcgtaacactttgaacgatata

SEQ ID NO: 1256 - DNA, 32 bp, Artificial Sequence
  Affymetrix ID 3879490
gaaaatgcaattgaccgtttggttaacatata

SEQ ID NO: 1257 - DNA, 35 bp, Artificial Sequence
  Affymetrix ID 3881731
tttggtcggattgtaaataaatataccattgtcct

SEQ ID NO: 1258 - DNA, 52 bp, Artificial Sequence
  Affymetrix ID 3881914
gcgttctgtcacgatgactgtattggaccctcaaagctctgtgtattgtgcc

SEQ ID NO: 1259 - DNA, 75 bp, Artificial Sequence
  Affymetrix ID 3882710
gatagataactcattggaaacgtgcatacatttatattcagatgaaattatggtttgcactgtctattaaatatc

SEQ ID NO: 1260 - DNA, 85 bp, Artificial Sequence
  Affymetrix ID 3882867
ggaccaagtccttacgtagaggtcacagtagatggacagtcaaagaagacagaaaaatgcaacaacacaaacagtcccaagtgga

SEQ ID NO: 1261 - DNA, 30 bp, Artificial Sequence
  Affymetrix ID 3883424
tgtgcgccatcattgggggcatgttcacag

SEQ ID NO: 1262 - DNA, 206 bp, Artificial Sequence
  Affymetrix ID 3883533
tggcactgctcaacttgttctcatcctaagcagggttgatagaagaacatcatgaggacgaagtggtaacatttcaagttgtcaaagggtaaagggaacaggaataagaaaatacaaaacaattttaaaactaattatttacttatagtttaacatggaaggctataaaaaaatttagatgggtatgtgtttaaccactttgttgc

SEQ ID NO: 1263 - DNA, 269 bp, Artificial Sequence
  Affymetrix ID 3883537
acagactggtcatgctttgttattcttggacccctgctttttcccaaaccactacattcacctttttgctataccaaactggaggtggtaattcttagttgagtcattttacctaggggaccacggtacagcgataagtcactcaattactaccttatgattgacagttcacactggaatcaaaggtgaattcatgggagtagaggtgagatatcgttaggcaagtctacaaagagagatagatgggcatttattcatcacgctgaagt

SEQ ID NO: 1264 - DNA, 91 bp, Artificial Sequence
  Affymetrix ID 3883987
actatcaatggcatttcaagtcttctgaaacagcatggctgtatgtgcgtggtccatagcacagtacatgcagcatctaataagagtttcc

SEQ ID NO: 1265 - DNA, 140 bp, Artificial Sequence
  Affymetrix ID 3884147
aagtccagattgtagttatacttttgcttgtttttcagtttccccaacacacagcagatacctggtgagctcagatagtctctttctctgacactgtgtaagaagctgtgaatattcctaacttacccagatgttgcttt

SEQ ID NO: 1266 - DNA, 29 bp, Artificial Sequence
  Affymetrix ID 3884334
gtatgtcttgtaacgtgcagaaccattgt

SEQ ID NO: 1267 - DNA, 31 bp, Artificial Sequence
  Affymetrix ID 3884673
attttacatgcttttaattcaaggtttgcag

SEQ ID NO: 1268 - DNA, 434 bp, Artificial Sequence
  Affymetrix ID 3884709
gttcattctgagacagcctttgtggtttctcttgatttcaccattctggtcccgtacttgattctctcatctgttcctttttaagcctaggttttaattttgtccctgaaatatccactaaagttactgcagtaactacatgtttttttatctttaatcttgcaatgttcatcttaaacaagtctgaccatataaaaataggaacattagggagaataactcagtatatggataaatattctgagtgaggaacacagcacctactacaagaggagctttttgctttatcttttttcttaccttatcagcaaatgttactttatctgtgatcaggtcagaagtgtaggaagattgacttagaacaaaacataacaaagacagaagtgtcagcttgactaaaggtgttcctccattgtacaggattttaggttagc

SEQ ID NO: 1269 - DNA, 33 bp, Artificial Sequence
  Affymetrix ID 3885711
gggaggatcaaagggaataaagagaactcttgg

SEQ ID NO: 1270 - DNA, 180 bp, Artificial Sequence
  Affymetrix ID 3886656
ttcctctgaggaaactcgaatcctgaaatggaaattctttgtggcagataactggcttatgacaccttgaaaagttcaagtgctcatataacacaccacactgaaccccctttcctacagcaatatgttcactatgttaccaatttgcaacttgtgcttcaatagtggaatctactttca

SEQ ID NO: 1271 - DNA, 102 bp, Artificial Sequence
  Affymetrix ID 3886907
acaacgagacattagaggtgcacccacccccgaccactacatatcaggacgtcatcctaggcactcggaagacctatgccatctatgacttgcttgacaccg

SEQ ID NO: 1272 - DNA, 42 bp, Artificial Sequence
  Affymetrix ID 3887661
ctggccagtgattcacgaaaacgcaaattgccatgtgatact

SEQ ID NO: 1273 - DNA, 110 bp, Artificial Sequence
  Affymetrix ID 3888081
gactcacgagatgttcatcaatgcaatcaagcaatatctctgtgtggccttgtccaaaaacggcgtctcttcagtgcctgatgtctttgagctctctcttgccatttttc

SEQ ID NO: 1274 - DNA, 45 bp, Artificial Sequence
  Affymetrix ID 3888182
ggaagttctccttttgaacttgtcacgaattccatcttgtaaagg

SEQ ID NO: 1275 - DNA, 73 bp, Artificial Sequence
  Affymetrix ID 3888253
tgtggtggtatggtacgtagctattttcctaagcatgtctgtcaatctcccttcttgctgattagctttcata

SEQ ID NO: 1276 - DNA, 43 bp, Artificial Sequence
  Affymetrix ID 3888289
ggctgtgcccacttcaagaatgtcattgttagactttgaaatt

SEQ ID NO: 1277 - DNA, 59 bp, Artificial Sequence
  Affymetrix ID 3888494
gcagagtccgtgcttgctatctgtctcatgttacagagcttccattacatattaaacgt

SEQ ID NO: 1278 - DNA, 195 bp, Artificial Sequence
  Affymetrix ID 3888936
ttataggtctcaatacagcaaaatgaaaacgaaaattgagaacattgctcattaggccagcaactttaaaattatttaatttgaaatataaaataggtggtcttcataaaaagatgcatgaaatttaccttaccttatattttatactttaagagtacattttatacaaatcagtaaccaggcttctttcatgtt

SEQ ID NO: 1279 - DNA, 1208 bp, Artificial Sequence
  Affymetrix ID 3889140
gccacgctggcaaacaatgaaagtagagtcgctcagaaacacgaaagatcatatgtgtgtcatcacagcatcgagaatttaaatcatctggaagttcctgctaaattaaagcatactgtgccagagctcccctctaatcaaaaaacgctgtcctggtgaaaatttgcaatgaggattacagagagagagatcaaccaatgaggaaatcacagactcttacatgagtttacagttaaccccactgcaacaaaataataaattagccataatttgttttttttgcaaataccatgccccccacctgaccccacaaacacaacagtcactgacatggcccagctatattaacagactgcgcctcaaaccatgctgtggcggagaagacagattcacgggtaatgccgctttggcctgagaagatgcttcggagctccaggtggaggagctgctcacgctgcaggatgctttgcaggttcattcaaaaccaaatttttttccaagaagaactggacataattcatgggaatattttaagtatatataagtggcaaaaaagctgtacattaaaagaagggggagaaataaaatgtgttgtatttacaaagctctttgtatatttttttaaatgaaaacaaagctgggagtgatagaattttaatgcaatgaaaaccaaataattcttcaaaaaactttagatgacatcattctctcgctctttcacacacacacacaccccccacacacttcaaacatacagaaaaggttaaagataaaaatagctgataggtgtttaagtaggatggagcagaagaatgtacatgtcccatttgcaattttggcaaaggtttaaaatgctgccctaaaggcacgcacgcatgcccagcctaccccagtaaaaacaagacaaagtataaaaagaaaagtctggcggggggtgggggaggcattagcatatcaatttccaatgagcacaaaattcaaaatacattaaaaagtagcatggcttacattttcagcagagaagaaactgaggaaatgttcataaagagacaatgatttttaagatttactgtcacaaaaaaaaaagggtttcacttcaggggagtggaactgggatgggaattttacaatgctagttccagcacgtggtctaatggccccaggggctggaaaagggaggtctgctccaactttaagatgcgcccttg

SEQ ID NO: 1280 - DNA, 74 bp, Artificial Sequence
  Affymetrix ID 3889782
agaagacagtattcacctatgtattttgcataacgattatatcaagtctaggggcttcatgtcatgttattaaa

SEQ ID NO: 1281 - DNA, 33 bp, Artificial Sequence
  Affymetrix ID 3891221
atgaggatcctgcatgttaatgggtttaatgga

SEQ ID NO: 1282 - DNA, 124 bp, Artificial Sequence
  Affymetrix ID 3891257
tcacccaccatagggcatgattaacaaagcaacctttcccttcccccgagtgattttgcgaaacccccttttcccttcagcttgcttagatgttccaaatttagaaagcttaaggcggcctaca

SEQ ID NO: 1283 - DNA, 37 bp, Artificial Sequence
  Affymetrix ID 3892672
taaaacttatgctatctgcggggccattcgtaggatg

SEQ ID NO: 1284 - DNA, 128 bp, Artificial Sequence
  Affymetrix ID 3894074
ccaattcttgaatatgttcttttcaaaatcttaagaaaagaaccttttttctttattaacatcatgtgtttactttcagcaaatatttgtattactgcttgattctgtgacagtcacaatagatgtag

SEQ ID NO: 1285 - DNA, 284 bp, Artificial Sequence
  Affymetrix ID 3894508
catgccaatgggtgtgtggtttctttttgaggtggtgaaaatgttcaggaattagaaatggtggtggatgtgcaacactgtgaacatactaaatccactgaattgtatactttaaacaggtatgtaaattatgggtatgtaaattatatctcaataaagctgctatttaaaaaaggaggaacaggtcacttagtgctgatgtttattgttaaggctccaatcttttgggttctacaaaatatacacctgaaacactccaacttgctccttgatgattactcatg

SEQ ID NO: 1286 - DNA, 28 bp, Artificial Sequence
  Affymetrix ID 3894602
caccatttatgagtctcaagttttatta

SEQ ID NO: 1287 - DNA, 34 bp, Artificial Sequence
  Affymetrix ID 3895000
cctgtttcccgtaaggctgtacatagtcctttta

SEQ ID NO: 1288 - DNA, 91 bp, Artificial Sequence
  Affymetrix ID 3896202
gatctggtctagttaacctagaagtatttttgtctcttagaaatacttgtgatttttataatacaaaagggtcttgactctaaatgcagtt

SEQ ID NO: 1289 - DNA, 128 bp, Artificial Sequence
  Affymetrix ID 3898306
ttggctgcgctaaactgaaaagttagtgaaacttcaaagttgggggattccaactgatatttttaaatcagcaatgtctaaggtgaccattaaaaattgtttttgttcctaggataactcatcttctc

SEQ ID NO: 1290 - DNA, 42 bp, Artificial Sequence
  Affymetrix ID 3901666
tgtatattttcacaagtatgtctacacactggtcatgatttt

SEQ ID NO: 1291 - DNA, 51 bp, Artificial Sequence
  Affymetrix ID 3902984
gtcagtggaaaacgtgtctctttaaagctgctatgtgaacagcttttacag

SEQ ID NO: 1292 - DNA, 25 bp, Artificial Sequence
  Affymetrix ID 3903290
agtggttggtctgaagtccttgcaa

SEQ ID NO: 1293 - DNA, 29 bp, Artificial Sequence
  Affymetrix ID 3904028
cttgcatcaacattcgagacatgatctga

SEQ ID NO: 1294 - DNA, 92 bp, Artificial Sequence
  Affymetrix ID 3904029
ctctcgctgaccttggacactcacccagggtgccaacccttcaatgcctgctcctggaagtctttcttacccatgtgagctaccccagagtc

SEQ ID NO: 1295 - DNA, 45 bp, Artificial Sequence
  Affymetrix ID 3904255
cgacgttcccgaagcaaaagtccattcagaaaagacaagagccct

SEQ ID NO: 1296 - DNA, 47 bp, Artificial Sequence
  Affymetrix ID 3904257
cagtgccatccgaggaaagattgggttgcctcatagcatcaaattaa

SEQ ID NO: 1297 - DNA, 145 bp, Artificial Sequence
  Affymetrix ID 3904276
atatttaaccacttcaacattgtaaatctggataacttttaatatctaaactatataagaaagtaaaatttaacatgttaatatggaaaatttttttatttttgtatcttgatgacttaaatgtaagcaacaaagtggttaaaca

SEQ ID NO: 1298 - DNA, 107 bp, Artificial Sequence
  Affymetrix ID 3904281
tcagagatgcattacgtgtaacttacaaaagtaaatatatgctattgatgtaataatattgtgcagtagtttcacttcagcgtgatgaataaatgcccatctatctc

SEQ ID NO: 1299 - DNA, 59 bp, Artificial Sequence
  Affymetrix ID 3906196
gatgatcctaacttttggcagaaatgggctaaaatagctgaactagacactgaagcaaa

SEQ ID NO: 1300 - DNA, 31 bp, Artificial Sequence
  Affymetrix ID 3907788
tgctggcaggaaataaatgaatgtttactga

SEQ ID NO: 1301 - DNA, 156 bp, Artificial Sequence
  Affymetrix ID 3908789
atgccactactgctaggttggtcccctcccaacttgcccctatcctttttttatttttaagaaaaaaactttgtaatgcttgtgattccatctgggcaaaagtctgaagatctgaaataactttatatcaaaccattgatcaataaacagctacta

SEQ ID NO: 1302 - DNA, 343 bp, Artificial Sequence
  Affymetrix ID 3909376
tgtagggcttaatggcacatgtaaagcaaataaagtatgttcactacttcagatgcaaatcctaaaataggaagaataagggcttattggctataagaagaaatagggttttaccgtgaccacagtttttgactcaatgagtgatatagacaggtattaaatatgttgctgaggtaaaggcaggaaggaaagaaaatgcacaaatacaagaatgatgtttttttctttcaacattttattttttagtttctgaaatacaaatcagaatacattttgttacataatcattttaacaagcttgacattttttcaatgtaagctgactcgatataggtactttaaa

SEQ ID NO: 1303 - DNA, 104 bp, Artificial Sequence
  Affymetrix ID 3911474
tgctacatagtcaaacagaactgggttggtcttttatggcataaaattaattcacagtcaaactctcaactagtactagaaggattcgctgaagagtttctctc

SEQ ID NO: 1304 - DNA, 151 bp, Artificial Sequence
  Affymetrix ID 3911564
cactaatcctaaatggggtgacccatttaaaatggcctctgtgtaaattcacagttcattaaatagagtctggtttgaacaggtcattatgtagttagtatataagtaatggattgagcaaggatggctggggatacccatggggacacac

SEQ ID NO: 1305 - DNA, 34 bp, Artificial Sequence
  Affymetrix ID 3911706
tccattaaacccattaacatgcaggatcctcatc

SEQ ID NO: 1306 - DNA, 239 bp, Artificial Sequence
  Affymetrix ID 3911738
gttgctttgttaatcatgccctatggtgggtgattaactgcttgtacaattacgtttcacttttaattaattgtgcttaaggctttaattaaatttgggggttcccttcttagagcagctcgtactgacgaaggtgcatgcgctgaatgatgtcacggcagtcgttgaacacacggcggatgttctcagtgtccacagcgcaggtgaaatgagggtagcagtagtgacgcccatctcca

SEQ ID NO: 1307 - DNA, 80 bp, Artificial Sequence
  Affymetrix ID 3911801
aagcagtgagagatgcactgaagacagaattcaaagcaaatgctgagaagacttctggcagcaacgtaaaaattgtgaaa

SEQ ID NO: 1308 - DNA, 176 bp, Artificial Sequence
  Affymetrix ID 3911815
cttgttctgaaaaaggtgtaatgccctaatatttttaaatccttatcctttgattttttaaaaactgacctattgataaaactataaattttcattttccagtgcagttagttacctttttaaacataaatgcacaacaagcaaatgaatttaagagaactaaactggaataagtg

SEQ ID NO: 1309 - DNA, 47 bp, Artificial Sequence
  Affymetrix ID 3913564
gctctggtaagtattcgtattctcttcatcccagtctgattgcatag

SEQ ID NO: 1310 - DNA, 37 bp, Artificial Sequence
  Affymetrix ID 3915125
aagcatatccagaatccatcgaacaattgaattaatg

SEQ ID NO: 1311 - DNA, 47 bp, Artificial Sequence
  Affymetrix ID 3917896
atgacctgtattttgccagacttaaatcacagatgggtattaaactt

SEQ ID NO: 1312 - DNA, 71 bp, Artificial Sequence
  Affymetrix ID 3918586
agttaaagcagcactacttacgtcatggaaaattggtgtctatagtccagtacattgtataaagaccacag

SEQ ID NO: 1313 - DNA, 33 bp, Artificial Sequence
  Affymetrix ID 3918651
cagacagctaccacttgctttatttcattacag

SEQ ID NO: 1314 - DNA, 246 bp, Artificial Sequence
  Affymetrix ID 3918723
cgcaagcgtaccagtgaatctcgttctagggcaagaaagagatcatctaagtccaagtctcatcgctctcagacacgttcacggtcacgttcaagacgcaggaggagaagcagcagatcaagatcaaagtctagaggaagaagatctgtatcaaaagagaagcgcaaaagatctccaaagcacagatccaagtctagggaaagaaaaagaaaaagatcaagctccagggataaccgaaagacagtt

SEQ ID NO: 1315 - DNA, 111 bp, Artificial Sequence
  Affymetrix ID 3918734
aacagatcgcacagagtaaagaagatgatgatgtaatagtgaataaacctcatgtttcggatgaagaggaagaagaacctcctttttatcatcatccctttaaactcagtg

SEQ ID NO: 1316 - DNA, 31 bp, Artificial Sequence
  Affymetrix ID 3919024
tatggacaacttttgctttgagtaataaact

SEQ ID NO: 1317 - DNA, 29 bp, Artificial Sequence
  Affymetrix ID 3920401
aaaactcaaagttgtatggattgtataga

SEQ ID NO: 1318 - DNA, 39 bp, Artificial Sequence
  Affymetrix ID 3920456
ctcaaagacggaattagattggttccttcaagatttgga

SEQ ID NO: 1319 - DNA, 1114 bp, Artificial Sequence
  Affymetrix ID 3920487
ctatgaacgtctcgaagcagtattctagtgataagaattcttaacatagccaagcgccccacgtttgttccccacgtttgttccccttttctgtttgaaaaacctgttctggtagctccacaagagagatgatactgactttttaaattttttacaagagtctgtattcctgatatgcctatatttttcctcaaagattctgcattttaaggatgggcataagcaaactatattttaataatttatagttaatgttaaaatattggctgatttagaccaaaagattcaaatctcctctttgtgaaatcccatctgcatttgattttttattattttatgttcccccgttagattgttttaagtgtttgcttttcatcttttatagatgtaatctgattttcaaaaatcattaacactttttaattagtatcgactaagactttttccccctggaatcgaggctgtgtgtccgtcatcccagcccccggttggagcctgctctttgaactccgctgccttccttagcagcttctgtcctcttctgtgagtcagtcagcgagtgcttgggatccgcatccagccgtgctgagcacacaacaggctgtgtgtggaaatggccaccaccattctccttccccaccccaccacaaaaagagaagctgtgtctttagacaaccctgaggtatctgtgttacaatcgttctgtgtttgatatttgtgtaaagtatgcatgcagtcttgtactgtgacctaagaacaaaactgtaactgcattagaaaccatgaaaaaattagatattgttttgtgacttttagacagtggtaaatatagaaccatgaattctggtcacattccatttctctccaacatgaaggatcaaaaaatgtttttcaatgtgttctttgttccactggaaacttagagtcatgagtttatgagctgatttggtcaccttcctctgcctttgttcactgtgagttctgatgtcttagtgacttagttcttagaagctcacgccttagtttgaaacagattctccacggtggtccccaaaacactgtctgcatatccataagaattgagcgctatgggtgttaacgtgcatgaggatca

SEQ ID NO: 1320 - DNA, 31 bp, Artificial Sequence
  Affymetrix ID 3922023
aatgtgacagcaacacgatgttttgatatgt

SEQ ID NO: 1321 - DNA, 39 bp, Artificial Sequence
  Affymetrix ID 3922993
attgctgctcagacaaatttgacactactccaagtcaac

SEQ ID NO: 1322 - DNA, 35 bp, Artificial Sequence
  Affymetrix ID 3924758
ctctgggaattctgtatatcacactaaaattttta

SEQ ID NO: 1323 - DNA, 258 bp, Artificial Sequence
  Affymetrix ID 3925641
ttctggacatgacgtctgtggtttaagctttgtgaaagaatgtgctttgattcgaagggtcttaaagaatttttttaatcgtcaaccacttttaaacataaagaattcacacaactactttcatgaattttttaatcccattgcaaacattattccaagagtatcccagtattagcaatactggaatataggcacattaccattcatagtaagaattctggtgtttacacaaccaaatttgatgcgatctgctcagta

SEQ ID NO: 1324 - DNA, 149 bp, Artificial Sequence
  Affymetrix ID 3926090
ccttgactgatctaacggaaatgctttagtaattttttgttcaactacttgattaccagtagtaattcgggagcaaatatttgatctctggttcaactaaattatcagtgagaactacttgaaaacgtattggtactatttggctgaca

SEQ ID NO: 1325 - DNA, 32 bp, Artificial Sequence
  Affymetrix ID 3927180
tagaagtttcagaataaacatgcatttcataa

SEQ ID NO: 1326 - DNA, 67 bp, Artificial Sequence
  Affymetrix ID 3927228
attttctgcaggatgattgtacagaatcattgcttatgacatgatcgctttctacactgtattacat

SEQ ID NO: 1327 - DNA, 673 bp, Artificial Sequence
  Affymetrix ID 3927229
cccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcg

SEQ ID NO: 1328 - DNA, 114 bp, Artificial Sequence
  Affymetrix ID 3928670
cataaggtcatgacgtgtctatgtcaaaagttcttatatatttcttttataagctgaaagaaggtctatttttatgtttttaggtctatgaatggaacgttgtaaatgcttgtc

SEQ ID NO: 1329 - DNA, 84 bp, Artificial Sequence
  Affymetrix ID 3929943
tttgcttgctgaaaatggtcgattaagcaatacccaaggagtcgtttctgccttttctaccatgatgagtgtccatcgcggaga

SEQ ID NO: 1330 - DNA, 31 bp, Artificial Sequence
  Affymetrix ID 3934699
aaagaagaaaacccgtatgctagatttgaaa

SEQ ID NO: 1331 - DNA, 136 bp, Artificial Sequence
  Affymetrix ID 3935246
tccctgggactcctagccatggtcagactgtacaaaacggttttccagaaatgaaatgtaaatccatttttatactgaaaatgttactgaaagtcacttttatgagcatctgccttaataaacagacattgattcc

SEQ ID NO: 1332 - DNA, 32 bp, Artificial Sequence
  Affymetrix ID 3935301
atgcttgatgcactgttggaaatgtgattaat

SEQ ID NO: 1333 - DNA, 28 bp, Artificial Sequence
  Affymetrix ID 3936897
acagaatcaggcatgctgttaataaata

SEQ ID NO: 1334 - DNA, 28 bp, Artificial Sequence
  Affymetrix ID 3939407
tttaaataaaaggcaacaggtcatgttc

SEQ ID NO: 1335 - DNA, 71 bp, Artificial Sequence
  Affymetrix ID 3942668
ttctccagaattattatgactattcagctcactttaacagttgaacttcaagcgacaatctttgaacaccc

SEQ ID NO: 1336 - DNA, 33 bp, Artificial Sequence
  Affymetrix ID 3945331
ttgagtggtttcatttacgtgaaggaggaggag

SEQ ID NO: 1337 - DNA, 32 bp, Artificial Sequence
  Affymetrix ID 3945370
actttcctgtttgcactactacttttttatta

SEQ ID NO: 1338 - DNA, 47 bp, Artificial Sequence
  Affymetrix ID 3946374
agaagaggaggtgtatcccctgttaaaaccatatgaaagcgtgatga

SEQ ID NO: 1339 - DNA, 49 bp, Artificial Sequence
  Affymetrix ID 3946589
gactttccctgctgttacctaattacaaattggatggaactgtgttttt

SEQ ID NO: 1340 - DNA, 42 bp, Artificial Sequence
  Affymetrix ID 3946677
cttgacaaggctgtatcagagcgtattgtccatgactacaag

SEQ ID NO: 1341 - DNA, 85 bp, Artificial Sequence
  Affymetrix ID 3953804
tgctgatctctactacacttccttcagtgaccctctctacctgaccatgttcaagatgctgcgtgacactctgtactacatgaag

SEQ ID NO: 1342 - DNA, 59 bp, Artificial Sequence
  Affymetrix ID 3956591
tgctagtgtagcttctgaaaggtgctttctccatttatttaaaactacccatgcaatta

SEQ ID NO: 1343 - DNA, 78 bp, Artificial Sequence
  Affymetrix ID 3957262
ggtggcacttggctaatagaatatggctgatattgtcattaatcattttgtaccttgacatgggttgtctaataaaac

SEQ ID NO: 1344 - DNA, 127 bp, Artificial Sequence
  Affymetrix ID 3959259
ctcaagcacagtcatgattggttttgtattttcttctccaggctacataataattttttgacctggttttcttgatgccttataattctgaatgctggacttgtctctttattacctgccagttagc

SEQ ID NO: 1345 - DNA, 33 bp, Artificial Sequence
  Affymetrix ID 3959453
ttgcagctgtcaccactacagtaagctggttta

SEQ ID NO: 1346 - DNA, 162 bp, Artificial Sequence
  Affymetrix ID 3960634
gtcatggaggactaaagcctaagtccttttcacttttcctccatctgaaggtaggtgagttcatcctcttcatggtaatgctgttttaccaagactttatagcagatggacccagaaagaattttctgctattgtgttcactacaacaggatagggacatca

SEQ ID NO: 1347 - DNA, 93 bp, Artificial Sequence
  Affymetrix ID 3961482
agcaattaacaatgattttcgtttaattctcttccaactctacagacataattctgctttcaccttcatcacgctttcatatggttttaacag

SEQ ID NO: 1348 - DNA, 30 bp, Artificial Sequence
  Affymetrix ID 3962268
ttcagtggaaagatgcacgttgatactatt

SEQ ID NO: 1349 - DNA, 26 bp, Artificial Sequence
  Affymetrix ID 3962596
cgctacttaatggaagaagatgacga

SEQ ID NO: 1350 - DNA, 38 bp, Artificial Sequence
  Affymetrix ID 3969510
tttaacaggcttttctcatgaagaactagacgactctt

SEQ ID NO: 1351 - DNA, 66 bp, Artificial Sequence
  Affymetrix ID 3969511
cttacataccgtgagaagttacgtaacatttactcctttgtaaatgtttccctatcatcagacaaa

SEQ ID NO: 1352 - DNA, 138 bp, Artificial Sequence
  Affymetrix ID 3970894
ggggctaccagacagtgttctcaacttggttaaggaggaagaaaacccagtcaatgaaattcaatgaaattcttggaaacttccattaagtgtgtagattgagcaggtagtaattgcatgcagtttgtacattagtgc

SEQ ID NO: 1353 - DNA, 152 bp, Artificial Sequence
  Affymetrix ID 3974757
ctaccgaatgcagatatggaaactcgaaggggtgcctacctcaatgctcttaaaatagccaagcttttgctaactgccattggctatggtcatgttcgagctgtggcagaagcttgtcagccaggtgtagaaggtgtgaatcccatgacaca

SEQ ID NO: 1354 - DNA, 66 bp, Artificial Sequence
  Affymetrix ID 3974789
aggcaaattatactcagtagtgtcacagctgatccgctgttgcaatgtctcttcaagaatgcagtc

SEQ ID NO: 1355 - DNA, 127 bp, Artificial Sequence
  Affymetrix ID 3981154
tagcagtaacacagctcttaatatgcctgttattcctatgaatactattgcagaagcagttattgaaatgattaaccgaggacagattcaaataacaattaatggattcagtattagcaatggactg

SEQ ID NO: 1356 - DNA, 81 bp, Artificial Sequence
  Affymetrix ID 3981752
tttaatgtgtccaacagaagaatcgatgaagcagaagaaagggctatcaaacttgtagataggccattggaaatcatgtaa

SEQ ID NO: 1357 - DNA, 250 bp, Artificial Sequence
  Affymetrix ID 3981906
ctgagtgatgagaaccaataatgcctataaccagtaacaaacaaaaagtatctgtgacctcacaaagggttgcttagaaaattataatctccaaactatagatcagaatgtagatctgatacaacctaacaggcatataaaatagcatcaggatataaggagagtaaggttgcagagttaactgtgaaactgtcaagttccaaagatggttcaggtacttctggatgctacatgactgggttgcattcta

SEQ ID NO: 1358 - DNA, 41 bp, Artificial Sequence
  Affymetrix ID 3982421
cagctcataattgatgccaaattaaagcactgtgtacccat

SEQ ID NO: 1359 - DNA, 39 bp, Artificial Sequence
  Affymetrix ID 3985558
agtttctgtcagcagtagtttcacccatttgcatggaaa

SEQ ID NO: 1360 - DNA, 149 bp, Artificial Sequence
  Affymetrix ID 3985635
ggccatgtgctttaacgttacggtaatactttactttaggcatccctcctgttgctagcagccttttgacctatctgcaatgcagtgttctcagtaggaaatgttcatctgttacatggaaaaaatgttgatggtgcattgtaaaatta

SEQ ID NO: 1361 - DNA, 737 bp, Artificial Sequence
  Affymetrix ID 3986857
tttttcacggtgctactaatcctgctgtatcccgggtttttaatataaaggtgttaagcttattttgctttgtaagtaaagaatgtgtatattgtgaacagccttttagctcaaaatgttgagtcatttacatatgacatagcatgaatcactctttacagaaaatgtaggaaaccctagaatacagacagcaatattttatattcatgtttatcaaagtgagaggacttatattcctacatcaagttactactgagagtaaatttattttgagttttatcccgtaagttctgttttgattttttttaaaaaacaaacccttttagtcactttaatcagaattttaaatgttcatgttacataccaaattataatatctaatggagcaatttgtcttttgctatattctccaagattatctcttaagaccatatgccccctgttttaatgtttcttacatcttgtttttactcatttctgactggacaaagttcttccaaacaattctgagaaacaaaaacacacacgcagaattaacaattcttttccctgtgcttcttatgtaagaatcctcctgtggcctctgcttgtacagaactgggaaacaacacttggttagtctcttttaagttacaaaaagccaattgatgtttcttattctttttaaattttaaatattttgttataaatactcacaggataccttatttccctagctatcatctcctgacttaatg

SEQ ID NO: 1362 - DNA, 108 bp, Artificial Sequence
  Affymetrix ID 3987532
tctgctttagatttatcccatatgctattgtttaatactggatgtatgtaagtgttttactgcactgtattgaattggtgtcttttgcacagttagcagtaaataaaa

SEQ ID NO: 1363 - DNA, 52 bp, Artificial Sequence
  Affymetrix ID 3988214
gtaatcaaagatgatatttggccaaacccattacagtactacttggttccca

SEQ ID NO: 1364 - DNA, 92 bp, Artificial Sequence
  Affymetrix ID 3989205
tgcacttgggctaaatatggatattgtgctgtatctgtgtttgtgtctgtgtgtgacagcatgaagataatgcctgtggttatgctgaataa

SEQ ID NO: 1365 - DNA, 69 bp, Artificial Sequence
  Affymetrix ID 3989770
aggtgactaacttgttgcagttgcctcagtactttgatttggaaatatataccactggacgattagaaa

SEQ ID NO: 1366 - DNA, 144 bp, Artificial Sequence
  Affymetrix ID 3991722
gtaagaagcagtcaattttcacatcaaagacagcatctaagaagttttgttctgtcctggaattattttagtagtgtttcagtaatgttgactgtattttccaacttgttcaaattattaccagtgaatctttgtcagcagttc

SEQ ID NO: 1367 - DNA, 67 bp, Artificial Sequence
  Affymetrix ID 3993347
cttccagaaaaatctacgtcttccaccaaatccaggtcttccagtcaatccacatcttccggaaaaa

SEQ ID NO: 1368 - DNA, 49 bp, Artificial Sequence
  Affymetrix ID 3993349
gtacgtcttccaacaaaggtatgtcttccaacaaaggtacgtcttccag

SEQ ID NO: 1369 - DNA, 186 bp, Artificial Sequence
  Affymetrix ID 4004822
ccttagcacgttatctcagggcttatgaaatgtaattaaatttattaagaaaatagatgaaaaattagggtacacagctggccaccaaatgcgaagtcaatctgctacttaaccctgaaaacaaaatcagttttgcatattaccactaacactaatacatatagagagcggaaccataactcattg

SEQ ID NO: 1370 - DNA, 33 bp, Artificial Sequence
  Affymetrix ID 4007593
tgaacaaatgtcaggatatccaatgccaaataa

SEQ ID NO: 1371 - DNA, 36 bp, Artificial Sequence
  Affymetrix ID 4009318
gcacagctaatgattcaatgcgagacaagattggcg

SEQ ID NO: 1372 - DNA, 104 bp, Artificial Sequence
  Affymetrix ID 4009451
tgaagtagatttgtgccgaaaagaatgtccgtttgtgatcaagccaaagatccagagacccaatactacacaagaaggagaggaaatggaaactgatatggatg

SEQ ID NO: 1373 - DNA, 141 bp, Artificial Sequence
  Affymetrix ID 4011604
attttttcagagaattcggatagccatagattatacagagccatttctcagcactcttttccttccacttaagaagctggaatacaacatgatgaggatgatgccaaaacaagactgctggttcatatcatctagggtaca

SEQ ID NO: 1374 - DNA, 358 bp, Artificial Sequence
  Affymetrix ID 4011967
gtctgccctagatccaggcctcgagctcccttcagcgaggcaccttacgcatggaataggaggtttcacccacctgaaaccttctaaatacaatatcaatggtgaaaaaccaaaaacaagagagaaaataaaaacaagatcattagtaaaagttcaacaaagcaattgaaattctttggatgtctattacatatgggaatgaggacagtctcaagtctgtaatatgggaaacatgcagaagggagagggttaatcagggctattgcatacagcagggatttttgctggaagaaaggctcttcattttctacaaagacaacttaattttgcttccatctactaggaatatgctggcttg

SEQ ID NO: 1375 - DNA, 41 bp, Artificial Sequence
  Affymetrix ID 4012169
tgcagcggttcattaaaatcgatggcaaggtccgaactgat

SEQ ID NO: 1376 - DNA, 202 bp, Artificial Sequence
  Affymetrix ID 4012627
ctaacctagtaatgaaagtgcaacaggagaattataaccagaaacatctaactgttatattatacatacatgtactgtttaactgtaattgtgaaaatgcattttgaaagttggaaaaaaagaaaggttgcctctatggtttggcagcttaataccgaaaagtagacagtgtcttgcgggtcatagatcatgggactcaaca

SEQ ID NO: 1377 - DNA, 227 bp, Artificial Sequence
  Affymetrix ID 4012632
tctgaatatttcaggggccctctgaaccatccagaaagccaggcagcagtaatgacaatttccagattgccataaattattttgtaaaaataactcaaaaggcaaaagcctttcatcagcctttactatgatatgaaaattttgttcaaagcaaaatttttcccttgcattagtttattaatgttaattccagttcgtttccatgaacccttatagattatcccgtc

SEQ ID NO: 1378 - DNA, 34 bp, Artificial Sequence
  Affymetrix ID 4012759
aaatgttgacaattgggacgatgtaaatgtaaag

SEQ ID NO: 1379 - DNA, 98 bp, Artificial Sequence
  Affymetrix ID 4013274
ggcacagttgataaagacacgaccatcatggaattaagagatcgacttcctaagaagcagcaagcaagtgcttccactgatggtgtcgataagctttc

SEQ ID NO: 1380 - DNA, 41 bp, Artificial Sequence
  Affymetrix ID 4013282
tcaagaaaacaggtagatagtgagcacatgcatcagaatgt

SEQ ID NO: 1381 - DNA, 40 bp, Artificial Sequence
  Affymetrix ID 4015535
ggtggcattaaagaaaacactgaaaaacatcagctaagag

SEQ ID NO: 1382 - DNA, 121 bp, Artificial Sequence
  Affymetrix ID 4016549
ttccatgtaacagatgaacatttcctactgagaacactgcattgcagataggtcaaaaggctgctagcaacaggagggatgcctaaagtaaagtattaccgtaacgttaaagcacatggcc

SEQ ID NO: 1383 - DNA, 38 bp, Artificial Sequence
  Affymetrix ID 4016573
gaggcaatgcattattgtgtagccacggttttctggaa

SEQ ID NO: 1384 - DNA, 578 bp, Artificial Sequence
  Affymetrix ID 4019367
aaccacttgactagcgactgtactgtttcctcgtggttcaggggtgtgcatgaaggctcttaggagagcaaacacctgttcctattctgtatgtccctccctcatttcaaatgagagtaaccaattgagtaaaataaccaaataaccattgccccaccatgaacatggggcttgggaagacagtcctacaatcttcatcatatatttaggtttttaggccagccagctctttttttccaaagctttcttttgaatgttcagatcctattttaatcctaactatagactactgtgtttgtgagggtgtctgagtgtctatgtgagggcaaggacaacagtgcagtccagaaacacagaaaatatgcttttttgcagctgagctctgttttgagatttcattttgttactggacagcgcttaatccataccaaagtctttggaacactgcagatttgctttagaggtagataaaacagaaatcatgcagttaagtcaattgagaaaaaaaaagggatttgttgtctttacagaacatcatgactaaaagttgatcctttgctcttggtgcacatttaaga

SEQ ID NO: 1385 - DNA, 25 bp, Artificial Sequence
  Affymetrix ID 4019418
cttcaatgatacctatatccatgtg

SEQ ID NO: 1386 - DNA, 84 bp, Artificial Sequence
  Affymetrix ID 4019610
ttattaagcactacataacacattgcatgctacataatacactgtaaccttctagcaggggtagatggccataactgagttatt

SEQ ID NO: 1387 - DNA, 306 bp, Artificial Sequence
  Affymetrix ID 4020462
atgatttctttactggtggaccaaatgaaattttgaaacagagccagttgacattactaactgcttttagatgtatagatttataatgaaatgtctgtggatattgggcaaataaggcactaatttttaagtttatattagccagtgtacctgtgtcctaaaattgtagcattgttttttagaatatactatattaatcaaaagtctcagctatgctgggagaagttccatttagttttattttttttctgatttgaatggatgttttatttgaagtagatatcggggttattctgtaatacgcta

SEQ ID NO: 1388 - DNA, 31 bp, Artificial Sequence
  Affymetrix ID 4024375
tggtggaagaccagtaattgctggaagactg

SEQ ID NO: 1389 - DNA, 96 bp, Artificial Sequence
  Affymetrix ID 4024376
tttctggaagaactagatttactggaagacttggatttggtggaagacacagatttttctggaagacatggattagctggaagatctgtatttgat

SEQ ID NO: 1390 - DNA, 25 bp, Artificial Sequence
  Affymetrix ID 4024377
tctggaagacactgactgactggaa

SEQ ID NO: 1391 - DNA, 39 bp, Artificial Sequence
  Affymetrix ID 4024378
agacctggattttttccggaagatgtggattgactggaa

SEQ ID NO: 1392 - DNA, 33 bp, Artificial Sequence
  Affymetrix ID 4024379
atggattttctggaagacgtggatcttcaggaa

SEQ ID NO: 1393 - DNA, 876 bp, Artificial Sequence
  Affymetrix ID 4030986
acagtagcttcaaaacggttcgatctgtttgttctcatgtagttttgtaaagatggaaaaaaaggactttggtcatcaagactactgtggccatattagattactggaacatctaagcatcagtgtgtgaccatgcgaacaaaagacttcggggagtgtctatttttaaaaaggtttctgtgtttcgaggcagttgtaaaagatttactgcagaatcaagcccacttttaggcttaggaccaggttctaactatctaaaaatattgactgataacaaaaagtattccaaatgtggctattctgatccatagttgttttttaaagaaaaaaaagtatatacagaaagagtataaaagtttgtgaatttaatgcaaattagcttccagtcttcacttcccaaatacttgatttgtagttttgactcctttacatttttgccttcttaatttcaagatttgcaattttgccttcaaaacagatctttttttttaattgtaaaaagtattcaacatatgcagaaataaaaagcattttgatggctgggcgcgggggctcaagcctgtaatcccagcactttgggaggccgaggcaggtggatcacctgaggtcaggagttcgaaaccagcctgggcaacatggtgaaaccctgtctctactaaaattacaaaaattagccgggcatggtggcaggtgcctgtaatcccagctacttgggaggctgaggcaggagaatcgcttgagcccgggaggcaaatggtacactgatctgagatcacaccactgcactccagcctgggcgacaaagtgagactgtctaaaaaataataataataaaaagcattttgaaattagtcgcggtcaatgcaattctactct

SEQ ID NO: 1394 - DNA, 118 bp, Artificial Sequence
  Affymetrix ID 4034997
tgactttgagctcccttgaattatcaggagaatcacagctggtaatgtactgcaagttcttgaagctacacaatatagtctggctaagttacatgtggtttccatattagcttctttg

SEQ ID NO: 1395 - DNA, 71 bp, Artificial Sequence
  Affymetrix ID 4035834
tgtttgtgtatttacgctttgattcatagtaacttcttatggaattgatttgcattgaacacaaactgtaa

SEQ ID NO: 1396 - DNA, 130 bp, Artificial Sequence
  Affymetrix ID 4035835
cacacactgatgcttagatgttccagtaatctaatatggccacagtagtcttgatgaccaaagtcctttttttccatctttacaaaactacatgagaacaaacagatcgaaccgttttgaagctactgtg

SEQ ID NO: 1397 - DNA, 25 bp, Artificial Sequence
  Affymetrix ID 4035838
gtggcttgagaaatatggacactta

SEQ ID NO: 1398 - DNA, 107 bp, Artificial Sequence
  Affymetrix ID 4035839
gactcaggccaagaaacgtcttctaaacttccccatcttctaaacccaatccaaatggcgtctggaagtccaatgtggcaaggaaaaacaggtcttcatcgaatcta

SEQ ID NO: 1399 - DNA, 69 bp, Artificial Sequence; identical to SEQ ID NO: 1138
  Affymetrix ID 4040526

SEQ ID NO: 1400 - DNA, 91 bp, Artificial Sequence
  Affymetrix ID 4041367
gaggccatcgatgacataccatttgggatcacttccaacagtgacgtgttctccaaataccagctcgacaaagatggggttgtcctcttta

SEQ ID NO: 1401 - DNA, 28 bp, Artificial Sequence
  Affymetrix ID 4041822
taaccatctaaacgctgggactttgata

SEQ ID NO: 1402 - DNA, 28 bp, Artificial Sequence
  Affymetrix ID 4043125
gcgtccttacaactttgaaatgtgcaat

SEQ ID NO: 1403 - DNA, 123 bp, Artificial Sequence
  Affymetrix ID 4044199
aagagacagagtaatttgcagtttgtttgatttatacttttgtttatctacaacccaataacagacatgagggatggccctgtctctctgggacagagcctcacagatgatgtccatgttttg

SEQ ID NO: 1404 - DNA, 43 bp, Artificial Sequence
  Affymetrix ID 4044413
tcacattcatatcatgtacattaagtactacataaacttgtct

SEQ ID NO: 1405 - DNA, 31 bp, Artificial Sequence; identical to SEQ ID NO: 363
  Affymetrix ID 4047577

SEQ ID NO: 1406 - DNA, 126 bp, Artificial Sequence
  Affymetrix ID 4052399
tatttggcttaaagaagcttgtgttactgaagttaacgcagaagtctggcctccgtttgtgggattattaaatatatatcctacgggatcagagtcatctattagaaacagtgatagccctgggta

SEQ ID NO: 1407 - DNA, 95 bp, Artificial Sequence
  Affymetrix ID 4055295
tcaagaaaacaggtagatagtgagcacatgcatcagaatgttccaacagaggaacaaagaacaaataaaagtaccggtggtgaacataagaaatc

SEQ ID NO: 1408 - DNA, 403 bp, Artificial Sequence
  Affymetrix ID 4055302
ggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtc

SEQ ID NO: 1409 - DNA, 32 bp, Artificial Sequence
  Affymetrix ID 2720180
aactgaaaattactatgcacttgtcagaaaca

SEQ ID NO: 1410 - DNA, 53 bp, Artificial Sequence
  Affymetrix ID 2347642
tttatagtttgaattccttcaactatacccacagtccaaaagcagactcactg

SEQ ID NO: 1411 - DNA, 67 bp, Artificial Sequence
  Affymetrix ID 2395344
atgcagaagacgaccctgtggatttttaggtctgttcagttccagcgctacactaaaatacaccaag

SEQ ID NO: 1412 - DNA, 307 bp, Artificial Sequence
  Affymetrix ID 2397069
ccagctgatctgacatggagccgcataccttggggattaagtaaatatgtactgtagaaaaccaaacctgcatgtaattccaggttgcttttgttttgttaaatactgccctgagatttacaattcctccggggtggctctgactgggactgaactttggagcctgaactatcagccaaagccgatccgattaactcttccctagccgggctggccggagcctgtggtggccaagctagcaggggtctggctgtccaggactccctcttctccttgggtggtttggacagccgcttgaccttgcatt

SEQ ID NO: 1413 - DNA, 522 bp, Artificial Sequence
  Affymetrix ID 2430377
ggcgctttggcttctgacacactcagaccttgggcctgtttctcaactttttctgaacctcagtgtttttgtgtattcaacagggataatattaattgaataattaggaggaacaaatgcaataatgtacataaagcatgcatagtagtgtttataaatagcagccaccactgctttgattagaaataataacagctaacatttgaggatttactgtgtgccttgaacgctgctaagctctttatatatattacatattgagtggtagaatcaggattcaaatccagatctgactccaaagcgcatgcttttataaaacataatttaaatatagaatatcaatgtaaattgcttatttagaaagtttattttcttatattttctcaaaaatatttattgtcaaatcagtgattctgtagaggggtaaatagctaaatttccttttaaaaattgtatgatgtgctgatttcaaattgatgtgaaatacagatttcatttaaaggcagtcatgccagattgtgc

SEQ ID NO: 1414 - DNA, 221 bp, Artificial Sequence
  Affymetrix ID 2448270
gaagcacaagtggatccgttcgttctactagtcctaatgtccagccttctatctctcaacctattttaactgttcagcaacaaacacaggctacagcttttgtgcaacccactcaacagagtcatcctcagattgagcctgccaatcaagagttatcttcaaacatagtagaggttgttcagagttcaccagttgagcggccttctacttccacagcagta

SEQ ID NO: 1415 - DNA, 52 bp, Artificial Sequence
  Affymetrix ID 2553581
ccaagtcatattccgtcatacaaccaactccaaggaactctaactcacttta

SEQ ID NO: 1416 - DNA, 472 bp, Artificial Sequence
  Affymetrix ID 2600700
tcgtcttcgccctctgaaattaaagaaatgaaaaaaaaaaacaatatctgcagcgttgcttggtgcacagattgctgaaactgtggggcttacagaaatgactgccggtcatttgaatgagacctggaacaaatcgtttctcagaagtacttttctgttcatcaccagtctgtaaaatacatgtacctatagaaatagaacactgcctctgagttttgatgctgtatttgctgccagacactgagcttctgagacatccctgattctctctccatttggaattacaaccattgtattttgtttgtggcataaattacagtcatctgtctttcactggaatgaagaccatgcctaggaacattttttaaggactcagctgtggcttttagggcttggttcataccatgggggaaaaaaaagtcctaggagaaagcgacgtggctcattagtgttgcctcttcagtgctcaagc

SEQ ID NO: 1417 - DNA, 121 bp, Artificial Sequence
  Affymetrix ID 2624519
ggctactacagcagatacccaggcagaaacatcgactctgagaggccccgaggctaccatcatccccaaggattcttggaggacgatgactcgcccgtttgctatgattcacggagatctc

SEQ ID NO: 1418 - DNA, 40 bp, Artificial Sequence
  Affymetrix ID 2651846
aagaagattgctttaaatcacatctttagggagacaggga

SEQ ID NO: 1419 - DNA, 88 bp, Artificial Sequence
  Affymetrix ID 2682441
acattctgaaagatcctcctagtgaagcaaacagcatacagtctgcaaatgctacaacaaagaccagcgaaacaaatcacacctcaag

SEQ ID NO: 1420 - DNA, 124 bp, Artificial Sequence
  Affymetrix ID 2704512
tatttgggtctagataacatcgtggggttagaaatactctcagtaccacctaagaggaaacacacacgagattcagttaattttcatgctaaatccaagtgtcatatcacaccaccagaaattc

SEQ ID NO: 1421 - DNA, 46 bp, Artificial Sequence
  Affymetrix ID 2704702
tattcaatctctggcacaatgcagcctctgtagaaaagatattagg

SEQ ID NO: 1422 - DNA, 30 bp, Artificial Sequence
  Affymetrix ID 2902832
tggacaagctgtgccgaggttgtctcccaa

SEQ ID NO: 1423 - DNA, 166 bp, Artificial Sequence
  Affymetrix ID 2918558
cattgggcaattgtcgtgcaaaagaaatagcaaattaccgtagaaacaaaatagtaagagcacagctagatggataaagatacagatgtgaaaatatagatgccctttgcatgaaaattcagttttcctcagcactagagacccagatactcctgaattagcatta

SEQ ID NO: 1424 - DNA, 66 bp, Artificial Sequence
  Affymetrix ID 3031661
gttcgcccttctcaacctgactaagtgggcgttagcactgtccgcccactgcatgggcactacctg

SEQ ID NO: 1425 - DNA, 156 bp, Artificial Sequence
  Affymetrix ID 3212761
cacctattctgtgaagtggtcagcatcaagccccttgtctccaagcagtttacaattagttgttgcatttctcgttttcccatagcattttggagtttatggcttcattagcatttatctttggtattataattacgtgtttactcctttctgctc

SEQ ID NO: 1426 - DNA, 42 bp, Artificial Sequence
  Affymetrix ID 3268183
ggtaggcacgcctgttctggtggtggaagagacaaattctgc

SEQ ID NO: 1427 - DNA, 42 bp, Artificial Sequence
  Affymetrix ID 3472753
atctgcgctgttctgggacccactctttctttgtggggacag

SEQ ID NO: 1428 - DNA, 335 bp, Artificial Sequence
  Affymetrix ID 3501557
cagacgcacttttgaacttagcatccccagtttgatgcacctggatacatgggctgtgcctgacctaaaacgtgtgtctgttcgtttctgaaaataggactgtgttgttctacagctatagcctacctccttggtcctctcctacatccatttgaaaaatctttgaaaatgggaagagatttttaaaactcagacaacggtcaacttgaggttcaaactggttctagtttgaagttctaaaattaactttctttaaaagaaactcagcatgatcagatttttaggctagcaagctaaatgtgctgcaatgtactgtggaaactacgatgactgtg

SEQ ID NO: 1429 - DNA, 71 bp, Artificial Sequence
  Affymetrix ID 3543621
atacctactcctcacacagtggccgccggtcagcgcgctagaccggagcagctggacgtcccaagggacct

SEQ ID NO: 1430 - DNA, 807 bp, Artificial Sequence
  Affymetrix ID 3551833
cttaccgttttgatgcttttagccactgctcattagatgactccagccccaacttacttcctaaaaaccacctcaagtccaacatggatgttttattattaacagcatctcaaaccaaatactgactactctgagccccaaaccagaacactgaagcttttttttttttttttttttgagatggagtctcactctgtcgcccaggctggagtgcagtggcacgacctcggctcactgcagcctcttccacccgggttcaagtgatctcatgcctcagcctcccaagtagctgggattacaggcgcctgccaccgtacccggctaatttttgtatttttcatagagacaaggtttcaccatcttggccaggctggtcttgaactcctaacctcatgatccacccgcctcggcctcccaaagggctgggattacaggtgtgagccaccgtgcctggccttttgaaactttaaattacaattgtgattctcccgtgtccacatggctggggaggcctcagaatcatggcaggaggcaaaaggcatttcttacttggtggcggcaagagaaaaatgaggtagaagcaaaagtggaaacccctgataaacccatctcagatctcgtgagacttactatcatgagaataacatgggaaagattggtccccatgattcagttacctccccccgggtcccacctacaacatgtgggaatcctgggagatagaattcaagttgagatttgggtgaggacgcagcgaaaccatgtcagagggtaagaggtcccagtggcagagtaaaagcatcattc

SEQ ID NO: 1431 - DNA, 441 bp, Artificial Sequence
  Affymetrix ID 3562041
gctactggaactactgcgactgctactacttccactgctggaactacttccactgctgctactggttgaactgctactagaactgctactgctactgcttcgacttctactcttgcttgttttatttctagctcgacctctacttctgctcctagttttggttttgctcttgctctctggatgtactgtcaagactggctctactggtacctcagtgagttttacagactctacaggaaaatccttcctctcaggtagtaaatgcccttgctcctgagtaacttggggtgttggctgtaaaacagctaatgacaaatcctcaggctgagagggaggctgggactggagtactggttgggactgggattgaagctgaagctggggctgagactgaggctgaggttgagccacaggctcaggttggggctcagattctttttc

SEQ ID NO: 1432 - DNA, 47 bp, Artificial Sequence
  Affymetrix ID 3661099
ccagtagagactccagtccagttatgaggtcaagcagcaccttgcca

SEQ ID NO: 1433 - DNA, 88 bp, Artificial Sequence
  Affymetrix ID 3667907
aggcacagcagcagatcagagcgagatggagggtcagagcgtagcagcagaagaaatgaacccgagagcccacgacatcgacctaaag

SEQ ID NO: 1434 - DNA, 250 bp, Artificial Sequence
  Affymetrix ID 3701840
gatggatgccttaagcgatggcccaggggtgcttggtggaagtcagcagggcatctggagctcatcccaatggtgtctctatagttattaatactgtaacgtttacttgtaaagagattatcattttagaaacatgctgtttttgaaacagatgtgtgatggatgttgtacatcctttgcttcttggtattcattcagagtggatttttagcccctgatctacaaatgtacattgttacagggctgcttc

SEQ ID NO: 1435 - DNA, 48 bp, Artificial Sequence
  Affymetrix ID 3840609
atgagtgatgtcttacgagatgaaaatgtgtacagtgactttaaagaa

SEQ ID NO: 1436 - DNA, 236 bp, Artificial Sequence
  Affymetrix ID 3889144
gttaactcacacactatggatgctgaatcagatcaaggataacagattggacttaggacactctctctccctctcctctcatttactggttacgatcccaggaatccagtgtaacctggataagcagggctggctggagacgatactgagcatttccgaatggagattattgtgtggataactttttaccaaatgtcactgtcttttcctcccaaaccgacaccaagagctttcta

SEQ ID NO: 1437 - DNA, 150 bp, Artificial Sequence
  Affymetrix ID 3889625
gatggcagtcgaaatagcttcattgaagtgtcagcactcatccatcaatcaatcacccacaaggaaaaatagcaacagtacaacggggtggcttttatgggatttactcatgggcatagggaatagcggctcaaatgtagttctgacatg

SEQ ID NO: 1438 - DNA, 53 bp, Artificial Sequence
  Affymetrix ID 3897081
ggcttggcacctttacgacaagtattttagccactgaccctgaatgctgtgga

SEQ ID NO: 1439 - DNA, 38 bp, Artificial Sequence
  Affymetrix ID 3902441
ctccttcttctgcttttgactgttactctgctcctggc

SEQ ID NO: 1440 - DNA, 194 bp, Artificial Sequence
  Affymetrix ID 4026042
cttggtccgcaatacaggggtcatgccaacgtctcttgccctgtttaccccttattttaggaaacaaattgccttatggcttaaaaaaatccttttggaagtgtgacgtgccagtagaaaagcatagacatcatgagtgaacagctcagtggcttttcacaagtaaacaggcccgtgtaaccaacacctagatc

(19) United States
(12) Patent Application Publication
(10) Pub. No.: US 2016/0258026 A1
(43) Pub. Date: Sep. 8, 2016

(54) CANCER BIOMARKERS AND CLASSIFIERS AND USES THEREOF

(71) Applicants: THE UNIVERSITY OF BRITISH COLUMBIA, Vancover (CA); GENOMEDX BIOSCIENCES, INC., Vancouver (CA)

(72) Inventors: Elai Davicioni, La Jolla, CA (US); Mercedeh Ghadessi, New Westminster (CA); Ismael Alfonso Vergara Correa, West Vancouver (CA); Lucia Lam, Burnaby (CA); Peter Black, Vancouver (CA)

(21) Appl. No.: 15/032,159

(22) PCT Filed: Nov. 4, 2014
(86) PCT No.: PCT/CA2014/000787

§ 371 Date: Apr. 26, 2016

Related U.S. Application Data
(60) Provisional application No. 61/899,648, filed on Nov. 4, 2013.

(51) Int. Cl. C12Q 1/68; G06F 19/20

(52) U.S. Cl. CPC C12Q 1/6886; G06F 19/20; C12Q 2600/106; C12Q 2600/118; C12Q 2600/158

(57) ABSTRACT

The present invention relates to methods, systems and kits for the diagnosis, prognosis and the determination of cancer progression of cancer in a subject. The invention also provides methods, systems and kits for determining the treatment modality of a cancer in a subject. The methods, systems and kits comprise expression-based analysis of biomarkers. Further disclosed herein, certain instances, are probe sets for use in assessing a cancer status in a subject. Further disclosed herein are classifiers for analyzing a cancer, such as, for example, bladder cancer.

### RELATED APPLICATIONS

This application claims priority to U.S. Provisional Patent Application Ser. No. 61/899,648, filed on Nov. 4, 2013, which is hereby incorporated by reference herein in its entirety.

### FIELD OF THE INVENTION

The present invention relates to methods, systems and kits for the diagnosis, prognosis and the determination of cancer progression of cancer in a subject. The invention also provides methods, systems and kits for determining the treatment modality of a cancer in a subject. The methods, systems and kits comprise expression-based analysis of biomarkers. Further disclosed herein, in certain instances, are probe sets for use in assessing a cancer status in a subject. Further disclosed herein are classifiers for analyzing a cancer, such as, for example, bladder cancer.

### BACKGROUND OF THE INVENTION

Cancer is the uncontrolled growth of abnormal cells anywhere in a body. The abnormal cells are termed cancer cells, malignant cells, or tumor cells. Many cancers and

the abnormal cells that compose the cancer tissue are further identified by the name of the tissue that the abnormal cells originated from (for example, bladder cancer, breast cancer, lung cancer, colon cancer, prostate cancer, pancreatic cancer, thyroid cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if breast cancer cells spread to a bone (or anywhere else), it can mean that the individual has metastatic breast cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment. Thus, provided herein are methods, compositions and systems for the analysis of coding and non-coding targets for the diagnosis, prognosis, and monitoring of a cancer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to methods, systems and kits for the diagnosis, prognosis and determination of cancer progression of cancer in a subject. In some embodiments, the present invention provides a method of diagnosing, prognosing, determining the progression of cancer, predicting a therapeutic regimen or predicting benefit from therapy in a subject, comprising (a) assaying an expression level in a sample from the subject for a plurality of targets, wherein the plurality of targets comprises one or more targets selected from Table 2B, Table 16 or SEQ ID NOs:1-1440; and (b) diagnosing, prognosing, determining the progression of cancer, predicting a therapeutic regimen or predicting benefit from therapy in the subject based on the expression levels of the plurality of targets. In some embodiments, the cancer is selected from the group consisting of a carcinoma, sarcoma, leukemia, lymphoma, myeloma, and a CNS tumor. In some embodiments, cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a bladder cancer. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a thyroid cancer. In some embodiments, the plurality of targets comprises a coding target. In some embodiments, the coding target is an exonic sequence. In some embodiments, the plurality of targets comprises a non-coding target. In some embodiments, the non-coding target comprises an intronic sequence or partially overlaps an intronic sequence. In some embodiments, the non-coding target comprises a sequence within the UTR or partially overlaps with a UTR sequence. In some embodiments, the target comprises a nucleic acid sequence. In some embodiments, the nucleic acid sequence is a DNA sequence. In some embodiments, the nucleic acid sequence is an RNA sequence. In some embodiments, the plurality of targets comprises at least 5 targets selected from Table 2B, Table 16 or SEQ ID NOs: 1-1440. In some embodiments, the plurality of targets comprises at least 10 targets selected from Table 2B, Table 16 or SEQ ID NOs: 1-1440. In some embodiments, the plurality of targets comprises at least 15 targets selected from Table 2B, Table 2B, Table 16 or SEQ ID NOs: 1-1440 or SEQ ID NOs: 1-1440. In some embodiments, the plurality of targets comprises at least 20 targets selected from Table 2B, Table 16 or SEQ ID NOs: 1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the diagnosing, prognosing, determining progression of cancer, predicting a therapeutic regimen or predicting benefit from therapy includes determining the malignancy of the cancer. In some embodiments, the diagnosing, prognosing, determining progression of cancer, predicting a therapeutic regimen or predicting benefit from therapy includes determining the stage of the cancer. In some embodiments, the diagnosing, prognosing, determining progression of cancer, predicting a therapeutic regimen or predicting benefit from therapy includes assessing the risk of cancer recurrence. In some embodiments, determining the treatment for the cancer includes determining the efficacy of treatment. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise sequencing the plurality of targets.

Further disclosed herein in some embodiments is a probe set for assessing a cancer status of a subject comprising a plurality of probes, wherein the probes in the set are capable of detecting an expression level of one or more targets selected from Table 2B, Table 16 or SEQ ID NOs:1-1440, wherein the expression level determines the cancer status of the subject with at least 40% specificity. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a bladder cancer. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a thyroid cancer. In some embodiments, the probe set further comprises a probe capable of detecting an expression level of at least one coding target. In some embodiments, the coding target is an exonic sequence. In some embodiments, the probe set further comprises a probe capable of detecting an expression level of at least one non-coding target. In some embodiments, the non-coding target is an intronic sequence or partially overlaps with an intronic sequence. In some embodiments, the non-coding target is a UTR sequence or partially overlaps with a UTR sequence. In some embodiments, assessing the cancer status includes assessing cancer recurrence risk. In some embodiments, assessing the cancer status includes determining a treatment modality. In some embodiments, assessing the cancer status includes determining the efficacy of treatment. In some embodiments, the target is a nucleic acid sequence. In some embodiments, the nucleic acid sequence is a DNA sequence. In some embodiments, the nucleic acid sequence is an RNA sequence. In some embodiments, the probes are between about 15 nucleotides and about 500 nucleotides in length. In some embodiments, the probes are between about 15 nucleotides and about 450 nucleotides in length. In some embodiments, the probes are between about 15 nucleotides and about 400 nucleotides in length. In some embodiments, the probes are between about 15 nucleotides and about 350 nucleotides in length. In some embodiments, the probes are between about 15 nucleotides and about 300 nucleotides in length. In some embodiments, the probes are between about 15 nucleotides and about 250 nucleotides in length. In some embodiments, the probes are between about 15 nucleotides and about 200 nucleotides in length. In some embodiments, the probes are at least 15 nucleotides in length. In some embodiments, the probes are at least 25 nucleotides in length. In some embodiments, the expression level determines the cancer status of the subject with at least 50% specificity. In some embodiments, the expression level determines the cancer status of the subject with at least 60% specificity. In some embodiments, the expression level determines the cancer status of the subject with at least 65% specificity. In some embodiments, the expression level determines the cancer status of the subject with at least 70% specificity. In some embodiments, the expression level determines the cancer status of the subject with at least 75% specificity. In some embodiments, the expression level determines the cancer status of the subject with at least 80% specificity. In some embodiments, the expression level determines the cancer status of the subject with at least 85% specificity. In some embodiments, the non-coding target is a non-coding RNA transcript and the non-coding RNA transcript is non-polyadenylated.

Further disclosed herein in some embodiments is a system for analyzing a cancer, comprising: (a) a probe set comprising a plurality of target sequences, wherein (i) the plurality of target sequences hybridizes to one or more targets selected from Table 2B, Table 16 or SEQ ID NOs:1-1440; or (ii) the plurality of target sequences comprises one or more target sequences selected from Table 2B, Table 16 or SEQ ID NOs:1-1440; and (b) a computer model or algorithm for analyzing an expression level and/or expression profile of the target hybridized to the probe in a sample from a subject suffering from a cancer. In some embodiments, the system further comprises an electronic memory for capturing and storing an expression profile. In some embodiments, the system further comprises a computer-processing device, optionally connected to a computer network. In some embodiments, the system further comprises a software module executed by the computer-processing device to analyze an expression profile. In some embodiments, the system further comprises a software module executed by the computer-processing device to compare the expression profile to a standard or control. In some embodiments, the system further comprises a software module executed by the computer-processing device to determine the expression level of the target. In some embodiments, the system further comprises a machine to isolate the target or the probe from the sample. In some embodiments, the system further comprises a machine to sequence the target or the probe. In some embodiments, the system further comprises a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the cancer is a bladder cancer. In some embodiments, the bladder cancer is non-invasive bladder cancer, muscle-invasive bladder cancer, or advanced bladder cancer. In some embodiments, the system further comprises a sequence for sequencing the plurality of targets. In some embodiments, the system further comprises an instrument for amplifying the plurality of targets. In some embodiments, the system further comprises a label for labeling the plurality of targets.

Further disclosed herein in some embodiments is a method of analyzing a cancer in an individual in need thereof, comprising: (a) obtaining an expression profile from a sample obtained from the individual, wherein the expression profile comprises one or more targets selected from Table 2B, Table 16 or SEQ ID NOs:1-1440; and (b) comparing the expression profile from the sample to an expression profile of a control or standard. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the method further comprises providing diagnostic or prognostic information to the individual about the cardiovascular disorder based on the comparison. In some embodiments, the method further comprises diagnosing the individual with a cancer if the expression profile of the sample (a) deviates from the control or standard from a healthy individual or population of healthy individuals, or (b) matches the control or standard from an individual or population of individuals who have or have had the cancer. In some embodiments, the method further comprises predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises quantifying the expression level of the plurality of targets. In some embodiments, the method further comprises labeling the plurality of targets. In some embodiments, assaying the expression level of a plurality of targets may comprise the use of a probe set. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets.

Disclosed herein in some embodiments is a method of diagnosing cancer in an individual in need thereof, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) diagnosing a cancer in the individual if the expression profile of the sample (i) deviates from the control or standard from a healthy individual or population of healthy individuals, or (ii) matches the control or standard from an individual or population of individuals who have or have had the cancer. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises quantifying the expression level of the plurality of targets. In some embodiments, the method further comprises labeling the plurality of targets. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets.

Further disclosed herein in some embodiments is a method of predicting whether an individual is susceptible to developing a cancer, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

Further disclosed herein in some embodiments is a method of predicting an individual's response to a treatment regimen for a cancer, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises quantifying the target, the probe, or any combination thereof. In some embodiments, the method further comprises labeling the target, the probe, or any combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

Disclosed herein in some embodiments is a method of prescribing a treatment regimen for a cancer to an individual in need thereof, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the plurality of targets comprises at least 30 targets selected from Table 2B, Table 16 or SEQ ID NOs:1-1440. In some embodiments, the plurality of targets comprises at least 35 targets selected from Table 2B, Table 16 or SEQ ID NOs:1-1440. In some embodiments, the plurality of targets comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer. In some embodiments, the method further comprises a software module executed by a computer-processing device to compare the expression profiles. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the method further comprises quantifying the expression level of the plurality of targets. In some embodiments, the method further comprises labeling the plurality of targets. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof. In some embodiments, obtaining the expression level may comprise the use of a classifier. The classifier may comprise a probe selection region (PSR). In some embodiments, the classifier may comprise the use of an algorithm. The algorithm may comprise a machine learning algorithm. In some embodiments, obtaining the expression level may also comprise sequencing the plurality of targets. In some embodiments, obtaining the expression level may also comprise amplifying the plurality of targets. In some embodiments, obtaining the expression level may also comprise quantifying the plurality of targets.

In other embodiments, the present invention provides a method of determining the risk of bladder cancer recurrence in a subject having bladder cancer, comprising: measuring the expression level, in a bladder tissue or cell sample isolated from a subject, for at least one marker selected from Table 2B, Table 16 or SEQ ID NOs:1-1440; wherein an elevated or reduced expression level of the at least one marker, over a control expression level in a corresponding normal bladder cancer tissue or bladder cancer cell sample, is indicative of an increased risk of bladder cancer recurrence. In some embodiments, the bladder cancer is non-invasive bladder cancer, muscle-invasive bladder cancer, or advanced bladder cancer.

Further disclosed herein is a classifier for analyzing a cancer, wherein the classifier has an AUC value of at least about 0.60. The AUC of the classifier may be at least about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

Further disclosed herein is a classifier for analyzing a cancer, wherein the classifier has an AUC value of at least about 0.60. The AUC of the classifier may be at least about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

Further disclosed herein is a method for analyzing a cancer, comprising use of one or more classifiers, wherein the significance of the one or more classifiers is based on one or more metrics selected from the group comprising T-test, P-value, KS (Kolmogorov Smimov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the one or more classifiers may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The significance of the one or more classifiers may be based on three or more metrics selected from the group comprising AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval).

The one or more metrics may comprise AUC. The one or more metrics may comprise AUC and AUC P-value. The one or more metrics may comprise AUC P-value and Wilcoxon Test P-value. The one or more metrics may comprise Wilcoxon Test P-value. The one or more metrics may comprise AUC and Univariable Analysis Odds Ratio P-value (uvaORPval). The one or more metrics may comprise multivariable analysis Odds Ratio P-value (mvaORPval) and Multivariable Analysis Hazard Ratio P-value (mvaHRPval). The one or more metrics may comprise AUC and Multivariable Analysis Hazard Ratio P-value (mvaHRPval). The one or more metrics may comprise Wilcoxon Test P-value and Multivariable Analysis Hazard Ratio P-value (mvaHRPval).

The clinical significance of the classifier may be based on the AUC value. The AUC of the classifier may be at least about about 0.60, 0.61, 0.62, 0.63, 0.64, 0.65, 0.66, 0.67, 0.68, 0.69, 0.70 or more. The AUC of the classifier may be at least about 0.71, 0.72, 0.73, 0.74, 0.75, 0.76, 0.77, 0.78, 0.79, 0.80 or more. The AUC of the classifier may be at least about 0.81, 0.82, 0.83, 0.84, 0.85, 0.86, 0.87, 0.88, 0.89, 0.90 or more. The AUC of the classifier may be at least about 0.91, 0.92, 0.93, 0.94, 0.95, 0.96, 0.97, 0.98, 0.99 or more. The 95% CI of a classifier or biomarker may be between about 1.10 to 1.70. In some instances, the difference in the range of the 95% CI for a biomarker or classifier is between about 0.25 to about 0.50, between about 0.27 to about 0.47, or between about 0.30 to about 0.45.

The clinical significance of the classifier may be based on Univariable Analysis Odds Ratio P-value (uvaORPval). The Univariable Analysis Odds Ratio P-value (uvaORPval) of the classifier may be between about 0-0.4. The Univariable Analysis Odds Ratio P-value (uvaORPval) of the classifier may be between about 0-0.3. The Univariable Analysis Odds Ratio P-value (uvaORPval) of the classifier may be between about 0-0.2. The Univariable Analysis Odds Ratio P-value (uvaORPval) of the classifier may be less than or equal to 0.25, 0.22, 0.21, 0.20, 0.19, 0.18, 0.17, 0.16, 0.15, 0.14, 0.13, 0.12, 0.11. The Univariable Analysis Odds Ratio P-value (uvaORPval) of the classifier may be less than or equal to 0.10, 0.09, 0.08, 0.07, 0.06, 0.05, 0.04, 0.03, 0.02, 0.01. The Univariable Analysis Odds Ratio P-value (uvaORPval) of the classifier may be less than or equal to 0.009, 0.008, 0.007, 0.006, 0.005, 0.004, 0.003, 0.002, 0.001.

The clinical significance of the classifier may be based on multivariable analysis Odds Ratio P-value (mvaORPval). The multivariable analysis Odds Ratio P-value (mvaORPval) of the classifier may be between about 0-1. The multivariable analysis Odds Ratio P-value (mvaORPval) of the classifier may be between about 0-0.9. The multivariable analysis Odds Ratio P-value (mvaORPval) of the classifier may be between about 0-0.8. The multivariable analysis Odds Ratio P-value (mvaORPval) of the classifier may be less than or equal to 0.90, 0.88, 0.86, 0.84, 0.82, 0.80. The multivariable analysis Odds Ratio P-value (mvaORPval) of the classifier may be less than or equal to 0.78, 0.76, 0.74, 0.72, 0.70, 0.68, 0.66, 0.64, 0.62, 0.60, 0.58, 0.56, 0.54, 0.52, 0.50. The multivariable analysis Odds Ratio P-value (mvaORPval) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The multivariable analysis Odds Ratio P-value (mvaORPval) of the classifier may be less than or equal to 0.10, 0.09, 0.08, 0.07, 0.06, 0.05, 0.04, 0.03, 0.02, 0.01. The multivariable analysis Odds Ratio P-value (mvaORPval) of the classifier may be less than or equal to 0.009, 0.008, 0.007, 0.006, 0.005, 0.004, 0.003, 0.002, 0.001.

The clinical significance of the classifier may be based on the Kaplan Meier P-value (KM P-value). The Kaplan Meier P-value (KM P-value) of the classifier may be between about 0-0.8. The Kaplan Meier P-value (KM P-value) of the classifier may be between about 0-0.7. The Kaplan Meier P-value (KM P-value) of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Kaplan Meier P-value (KM P-value) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Kaplan Meier P-value (KM P-value) of the classifier may be less than or equal to 0.10, 0.09, 0.08, 0.07, 0.06, 0.05, 0.04, 0.03, 0.02, 0.01. The Kaplan Meier P-value (KM P-value) of the classifier may be less than or equal to 0.009, 0.008, 0.007, 0.006, 0.005, 0.004, 0.003, 0.002, 0.001.

The clinical significance of the classifier may be based on the survival AUC value (survAUC). The survival AUC value (survAUC) of the classifier may be between about 0-1. The survival AUC value (survAUC) of the classifier may be between about 0-0.9. The survival AUC value (survAUC) of the classifier may be less than or equal to 1, 0.98, 0.96, 0.94, 0.92, 0.90, 0.88, 0.86, 0.84, 0.82, 0.80. The survival AUC value (survAUC) of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The survival AUC value (survAUC) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The survival AUC value (survAUC) of the classifier may be less than or equal to 0.10, 0.09, 0.08, 0.07, 0.06, 0.05, 0.04, 0.03, 0.02, 0.01. The survival AUC value (survAUC) of the classifier may be less than or equal to 0.009, 0.008, 0.007, 0.006, 0.005, 0.004, 0.003, 0.002, 0.001.

The clinical significance of the classifier may be based on the Univariable Analysis Hazard Ratio P-value (uvaHRPval). The Univariable Analysis Hazard Ratio P-value (uvaHRPval) of the classifier may be between about 0-0.4. The Univariable Analysis Hazard Ratio P-value (uvaHRPval) of the classifier may be between about 0-0.3. The Univariable Analysis Hazard Ratio P-value (uvaHRPval) of the classifier may be less than or equal to 0.40, 0.38, 0.36, 0.34, 0.32. The Univariable Analysis Hazard Ratio P-value (uvaHRPval) of the classifier may be less than or equal to 0.30, 0.29, 0.28, 0.27, 0.26, 0.25, 0.24, 0.23, 0.22, 0.21, 0.20. The Univariable Analysis Hazard Ratio P-value (uvaHRPval) of the classifier may be less than or equal to 0.19, 0.18, 0.17, 0.16, 0.15, 0.14, 0.13, 0.12, 0.11. The Univariable Analysis Hazard Ratio P-value (uvaHRPval) of the classifier may be less than or equal to 0.10, 0.09, 0.08, 0.07, 0.06, 0.05, 0.04, 0.03, 0.02, 0.01. The Univariable Analysis Hazard Ratio P-value (uvaHRPval) of the classifier may be less than or equal to 0.009, 0.008, 0.007, 0.006, 0.005, 0.004, 0.003, 0.002, 0.001.

The clinical significance of the classifier may be based on the Multivariable Analysis Hazard Ratio P-value (mvaHRPval). The Multivariable Analysis Hazard Ratio P-value (mvaHRPval) of the classifier may be between about 0-1. The Multivariable Analysis Hazard Ratio P-value (mvaHRPval) of the classifier may be between about 0-0.9. The Multivariable Analysis Hazard Ratio P-value (mvaHRPval) of the classifier may be less than or equal to 1, 0.98, 0.96, 0.94, 0.92, 0.90, 0.88, 0.86, 0.84, 0.82, 0.80. The Multivariable Analysis Hazard Ratio P-value (mvaHRPval) of the classifier may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Multivariable Analysis Hazard Ratio P-value (mvaHRPval) of the classifier may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Multivariable Analysis Hazard Ratio P-value (mvaHRPval) of the classifier may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier may be less than or equal to 0.009, 0.008, 0.007, 0.006, 0.005, 0.004, 0.003, 0.002, 0.001.

The clinical significance of the classifier may be based on the Multivariable Analysis Hazard Ratio P-value (mvaHRPval). The Multivariable Analysis Hazard Ratio P-value (mvaHRPval) of the classifier may be between about 0 to about 0.60. significance of the classifier may be based on the Multivariable Analysis Hazard Ratio P-value (mvaHRPval). The Multivariable Analysis Hazard Ratio P-value (mvaHRPval) of the classifier may be between about 0 to about 0.50. significance of the classifier may be based on the Multivariable Analysis Hazard Ratio P-value (mvaHRPval). The Multivariable Analysis Hazard Ratio P-value (mvaHRPval) of the classifier may be less than or equal to 0.50, 0.47, 0.45, 0.43, 0.40, 0.38, 0.35, 0.33, 0.30, 0.28, 0.25, 0.22, 0.20, 0.18, 0.16, 0.15, 0.14, 0.13, 0.12, 0.11, 0.10. The Multivariable Analysis Hazard Ratio P-value (mvaHRPval) of the classifier may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier may be less than or equal to 0.01, 0.009, 0.008, 0.007, 0.006, 0.005, 0.004, 0.003, 0.002, 0.001.

The method may further comprise determining an expression profile based on the one or more classifiers. The method may further comprise providing a sample from a subject. The subject may be a healthy subject. The subject may be suffering from a cancer or suspected of suffering from a cancer. The method may further comprise diagnosing a cancer in a subject based on the expression profile or classifier. The method may further comprise treating a cancer in a subject in need thereof based on the expression profile or classifier. The method may further comprise determining a treatment regimen for a cancer in a subject in need thereof based on the expression profile or classifier. The method may further comprise prognosing a cancer in a subject based on the expression profile or classifier.

Further disclosed herein is a kit for analyzing a cancer, comprising (a) a probe set comprising a plurality of target sequences, wherein the plurality of target sequences comprises at least one target sequence listed in Table 2B, Table 16 or SEQ ID NOs:1-1440; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), frozen robust multichip average (fRMA), Single Channel Array Normalization (SCAN), ComBat (Combining Batches of gene expression), probe logarithmic intensity error estimation (PLIER), non-linear fit (NLFIT) quantile-based, nonlinear normalization, or a combination thereof. In some embodiments, the plurality of target sequences comprises at least 5 target sequences selected from Table 2B, Table 16 or SEQ ID NOs:1-1440. In some embodiments, the plurality of target sequences comprises at least 10 target sequences selected from Table 2B, Table 16 or SEQ ID NOs:1-1440. In some embodiments, the plurality of target sequences comprises at least 15 target sequences selected from Table 2B, Table 16 or SEQ ID NOs:1-1440. In some embodiments, the plurality of target sequences comprises at least 20 target sequences selected from Table 2B, Table 16 or SEQ ID NOs:1-1440. In some embodiments, the plurality of target sequences comprises at least 30 target sequences selected from Table 2B, Table 16 or SEQ ID NOs:1-1440. In some embodiments, the plurality of target sequences comprises at least 35 target sequences selected from Table 2B, Table 16 or SEQ ID NOs:1-1440. In some embodiments, the plurality of targets comprises at least 40 target sequences selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer.

Further disclosed herein is a kit for analyzing a cancer, comprising (a) a probe set comprising a plurality of target sequences, wherein the plurality of target sequences hybridizes to one or more targets selected from Table 2B, Table 16 or SEQ ID NOs:1-1440; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), frozen robust multichip average (fRMA), Single Channel Array Normalization (SCAN), ComBat (Combining Batches of gene expression), probe logarithmic intensity error estimation (PLIER), non-linear fit (NLFIT) quantile-based, nonlinear normalization, or a combination thereof. In some embodiments, the targets comprise at least 5 targets selected from Table 2B, Table 16 or SEQ ID NOs:1-1440. In some embodiments, the targets comprise at least 10 targets selected from Table 2B, Table 16 or SEQ ID NOs:1-1440. In some embodiments, the targets comprise at least 15 targets selected from Table 2B, Table 16 or SEQ ID NOs:1-1440. In some embodiments, the targets comprise at least 20 targets selected from Table 2B, Table 16 or SEQ ID NOs:1-1440. In some embodiments, the targets comprise at least 30 targets selected from Table 2B, Table 16 or SEQ ID NOs:1-1440. In some embodiments, the targets comprise at least 35 targets selected from Table 2B, Table 16 or SEQ ID NOs:1-1440. In some embodiments, the targets comprise comprises at least 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a breast cancer. In some embodiments, the cancer is a thyroid cancer. In some embodiments, the cancer is a lung cancer.

In other embodiments, the present invention provides methods for analyzing a sample from a subject comprising (a) assaying an expression level in a sample from the subject for a plurality of targets, wherein the plurality of targets comprises one or more targets selected from Table 2B, Table 16 or SEQ ID NOs:1-1440; and (b) comparing the expression level of the plurality of targets in the sample to a control level of the plurality of targets in a control sample. In other embodiments, the methods of the present invention further comprise a computer model or algorithm for analyzing the plurality of targets in the sample.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows AUC of receiver operating characteristics (ROC) of single clinical variables and classifiers in the discovery and validation set.

FIGS. 2A-C show survival ROC curves for various classifiers of the present invention. FIG. 2A shows survival ROC curves for GC and single clinical variables. FIG. 2B shows survival ROC curves for IBCNC compared to G-IBCNC. FIG. 2C shows survival ROC curves for CC compared to G-CC at 4 years in the validation set.

FIGS. 3A-B show decision curves of classifiers of the present invention. FIG. 3A shows IBCNC compared to G-IBCNC. FIG. 3B shows CC compared to G-CC in the validation set.

FIGS. 4A-E show discrimination plots in validation set. Classifier scores of cases and controls across IBCNC, CC, GC, G-IBCNC and G-CC models.

FIGS. 5A-B show survival AUC over time and Cumulative incidence plots of GC in LNI negative patients from the validation set.

FIG. 6 shows cumulative incidence plot comparing patients with high or low G-CC as determined by majority rule (cutoff=0.5) in the validation set.

FIGS. 7A-B show reclassification by G-IBCNC and G-CC compared to IBCNC.

FIG. 8 shows genomic classifier and tumor stage relation.

FIGS. 9A-B show GC scores discrimination in LNI negative or positive subsets compared to CC.

FIGS. 10A-B show cumulative incidence plots of (A) CC and (B) GC in LNI negative patients from the validation set.

FIG. 11 shows survival AUC performance of external signatures in comparison to GC at predicting recurrence at 4 years.

FIG. 12 shows GO terms associated with the 15 features in GC.

### DETAILED DESCRIPTION OF THE INVENTION

The present invention discloses systems and methods for diagnosing, predicting, and/or monitoring the status or outcome of a cancer in a subject using expression-based analysis of a plurality of targets. Generally, the method comprises (a) optionally providing a sample from a subject; (b) assaying the expression level for a plurality of targets in the sample; and (c) diagnosing, predicting and/or monitoring the status or outcome of a cancer based on the expression level of the plurality of targets.

Assaying the expression level for a plurality of targets in the sample may comprise applying the sample to a microarray. In some instances, assaying the expression level may comprise the use of an algorithm. The algorithm may be used to produce a classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

In some instances, the plurality of targets comprises one or more targets selected from Table 2B, Table 16 or SEQ ID NOs:1-1440. In some instances, the plurality of targets comprises at least about 2, at least about 3, at least about 4, at least about 5, at least about 6, at least about 7, at least about 8, at least about 9, or at least about 10 targets selected from Table 2B, Table 16 or SEQ ID NOs:1-1440. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some instances, the plurality of targets comprises a coding target, non-coding target, or any combination thereof. In some instances, the coding target comprises an exonic sequence. In other instances, the non-coding target comprises a non-exonic or exonic sequence. In some instances, the non-exonic sequence comprises an untranslated region (e.g., UTR), intronic region, intergenic region, antisense, or any combination thereof. Alternatively, the plurality of targets comprises an anti-sense sequence. In other instances, the plurality of targets comprises a non-coding RNA transcript.

Further disclosed herein, is a probe set for diagnosing, predicting, and/or monitoring a cancer in a subject. In some instances, the probe set comprises a plurality of probes capable of detecting an expression level of one or more targets selected from Table 2B, Table 16 or SEQ ID NOs:1-1440, wherein the expression level determines the cancer status of the subject with at least about 45% specificity. In some instances, detecting an expression level comprise detecting gene expression, protein expression, or any combination thereof. In some instances, the plurality of targets comprises one or more targets selected from Table 2B, Table 16 or SEQ ID NOs:1-1440. In some instances, the plurality of targets comprises at least about 2, at least about 3, at least about 4, at least about 5, at least about 6, at least about 7, at least about 8, at least about 9, or at least about 10 targets selected from Table 2B, Table 16 or SEQ ID NOs:1-1440. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:I-15. In some instances, the plurality of targets comprises a coding target, non-coding target, or any combination thereof. In some instances, the coding target comprises an exonic sequence. In other instances, the non-coding target comprises a non-exonic or exonic sequence. In some instances, the non-exonic sequence comprises an untranslated region (e.g., UTR), intronic region, intergenic region, antisense or any combination thereof. Alternatively, the plurality of targets comprises an anti-sense sequence. In other instances, the plurality of targets comprises a non-coding RNA transcript.

Further disclosed herein are methods for characterizing a patient population. Generally, the method comprises: (a) providing a sample from a subject; (b) assaying the expression level for a plurality of targets in the sample; and (c) characterizing the subject based on the expression level of the plurality of targets. In some instances, the plurality of targets comprises one or more targets selected from Table 2B, Table 16 or SEQ ID NOs:1-1440. In some instances, the plurality of targets comprises at least about 2, at least about 3, at least about 4, at least about 5, at least about 6, at least about 7, at least about 8, at least about 9, or at least about 10 targets selected from Table 2B, Table 16 or SEQ ID NOs:1-1440. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 2B, Table 16 or SEQ ID NOs:1-1440. In some embodiments, the plurality of targets comprises at least 50 targets selected from Table 2B, Table 16 or SEQ ID NOs:1-1440. In some embodiments, the plurality of targets comprises at least 60 targets selected from Table 2B, Table 16 or SEQ ID NOs:1-1440. In some embodiments, the plurality of targets comprises at least 100 targets selected from Table 2B, Table 16 or SEQ ID NOs:1-1440. In some embodiments, the plurality of targets comprises at least 125 targets selected from Table 2B, Table 16 or SEQ ID NOs:1-1440. In some embodiments, the plurality of targets comprises at least 150 targets selected from Table 2B, Table 16 or SEQ ID NOs:1-1440. In some embodiments, the plurality of targets comprises at least 175 targets selected from Table 2B, Table 16 or SEQ ID NOs:1-1440. In some embodiments, the plurality of targets comprises at least 200 targets selected from Table 2B, Table 16 or SEQ ID NOs:1-1440. In some embodiments, the plurality of targets comprises at least 225 targets selected from Table 2B, Table 16 or SEQ ID NOs:1-1440. In some embodiments, the plurality of targets comprises at least 250 targets selected from Table 2B, Table 16 or SEQ ID NOs:1-1440. In some embodiments, the plurality of targets comprises at least 275 targets selected from Table 2B, Table 16 or SEQ ID NOs:1-1440. In some embodiments, the plurality of targets comprises at least 300 targets selected from Table 2B, Table 16 or SEQ ID NOs:1-1440. In some embodiments, the plurality of targets comprises at least 350 targets selected from Table 2B, Table 16 or SEQ ID NOs:1-1440. In some embodiments, the plurality of targets comprises at least 400 targets selected from Table 2B, Table 16 or SEQ ID NOs:1-1440. In some embodiments, the plurality of targets comprises at least 450 targets selected from Table 2B, Table 16 or SEQ ID NOs:1-1440. In some embodiments, the plurality of targets comprises at least 500 targets selected from Table 2B, Table 16 or SEQ ID NOs:1-1440. In some embodiments, the plurality of targets comprises at least 550 targets selected from Table 2B, Table 16 or SEQ ID NOs:1-1440. In some embodiments, the plurality of targets comprises at least 600 targets selected from Table 2B, Table 16 or SEQ ID NOs:1-1440. In some embodiments, the plurality of targets comprises at least 650 targets selected from Table 2B, Table 16 or SEQ ID NOs:1-1440. In some embodiments, the plurality of targets comprises at least 700 targets selected from Table 2B, Table 16 or SEQ ID NOs:1-1440. In some embodiments, the plurality of targets comprises at least 750 targets selected from Table 2B, Table 16 or SEQ ID NOs:1-1440. In some embodiments, the plurality of targets comprises at least 800 targets selected from Table 2B, Table 16 or SEQ ID NOs:1-1440. In some embodiments, the plurality of targets comprises SEQ ID NOs:1-15. In some instances, the plurality of targets comprises a coding target, non-coding target, or any combination thereof. In some instances, the coding target comprises an exonic sequence. In other instances, the non-coding target comprises a non-exonic or exonic sequence. In some instances, the non-exonic sequence comprises an untranslated region (e.g., UTR), intronic region, antisense, intergenic region, or any combination thereof. Alternatively, the plurality of targets comprises an anti-sense sequence. In other instances, the plurality of targets comprises a non-coding RNA transcript.

In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### DEFINITIONS

Unless defined otherwise or the context clearly dictates otherwise, all technical and scientific terms used herein have the same meaning as commonly understood by one of ordinary skill in the art to which this invention belongs. In describing the present invention, the following terms may be employed, and are intended to be defined as indicated below.

The term “polynucleotide” as used herein refers to a polymer of greater than one nucleotide in length of ribonucleic acid (RNA), deoxyribonucleic acid (DNA), hybrid RNA/DNA, modified RNA or DNA, or RNA or DNA mimetics, including peptide nucleic acids (PNAs). The polynucleotides may be single- or double-stranded. The term includes polynucleotides composed of naturally-occurring nucleobases, sugars and covalent internucleoside (backbone) linkages as well as polynucleotides having non-naturally-occurring portions which function similarly. Such modified or substituted polynucleotides are well known in the art and for the purposes of the present invention, are referred to as “analogues.”

“Complementary” or “substantially complementary” refers to the ability to hybridize or base pair between nucleotides or nucleic acids, such as, for instance, between a sensor peptide nucleic acid or polynucleotide and a target polynucleotide. Complementary nucleotides are, generally, A and T (or A and U), or C and G. Two single-stranded polynucleotides or PNAs are said to be substantially complementary when the bases of one strand, optimally aligned and compared and with appropriate insertions or deletions, pair with at least about 80% of the bases of the other strand, usually at least about 90% to 95%, and more preferably from about 98 to 100%.

Alternatively, substantial complementarity exists when a polynucleotide may hybridize under selective hybridization conditions to its complement. Typically, selective hybridization may occur when there is at least about 65% complementarity over a stretch of at least 14 to 25 bases, for example at least about 75%, or at least about 90% complementarity.

“Preferential binding” or “preferential hybridization” refers to the increased propensity of one polynucleotide to bind to its complement in a sample as compared to a noncomplementary polymer in the sample.

Hybridization conditions may typically include salt concentrations of less than about 1 M, more usually less than about 500 mM, for example less than about 200 mM. In the case of hybridization between a peptide nucleic acid and a polynucleotide, the hybridization can be done in solutions containing little or no salt. Hybridization temperatures can be as low as 5° C., but are typically greater than 22° C., and more typically greater than about 30° C., for example in excess of about 37° C. Longer fragments may require higher hybridization temperatures for specific hybridization as is known in the art. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art.

“Multiplexing” herein refers to an assay or other analytical method in which multiple analytes are assayed. In some instances, the multiple analytes are from the same sample. In some instances, the multiple analytes are assayed simultaneously. Alternatively, the multiple analytes are assayed sequentially. In some instances, assaying the multiple analytes occurs in the same reaction volume. Alternatively, assaying the multiple analytes occurs in separate or multiple reaction volumes.

A “target sequence” as used herein (also occasionally referred to as a “PSR” or “probe selection region”) refers to a region of the genome against which one or more probes can be designed. A “target sequence” may be a coding target or a non-coding target. A “target sequence” may comprise exonic and/or non-exonic sequences. Alternatively, a “target sequence” may comprise an ultraconserved region. An ultraconserved region is generally a sequence that is at least 200 base pairs and is conserved across multiple species. An ultraconserved region may be exonic or non-exonic. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. The term “target sequence” is used interchangeably with the terms “target”, “marker”, and “biomarker” throughout the specification.

As used herein, a probe is any polynucleotide capable of selectively hybridizing to a target sequence or its complement, or to an RNA version of either. A probe may comprise ribonucleotides, deoxyribonucleotides, peptide nucleic acids, and combinations thereof. A probe may optionally comprise one or more labels. In some embodiments, a probe may be used to amplify one or both strands of a target sequence or an RNA form thereof, acting as a sole primer in an amplification reaction or as a member of a set of primers.

As used herein, a non-coding target may comprise a nucleotide sequence. The nucleotide sequence is a DNA or RNA sequence. A non-coding target may include a UTR sequence, an intronic sequence, antisense, or a non-coding RNA transcript. A non-coding target also includes sequences which partially overlap with a UTR sequence or an intronic sequence. A non-coding target also includes non-exonic or exonic transcripts.

As used herein, a coding target includes nucleotide sequences that encode for a protein and peptide sequences. The nucleotide sequence is a DNA or RNA sequence. The coding target includes protein-coding sequence. Protein-coding sequences include exon-coding sequences (e.g., exonic sequences).

As used herein, diagnosis of cancer may include the identification of cancer in a subject, determining the malignancy of the cancer, or determining the stage of the cancer.

As used herein, prognosis of cancer may include predicting the clinical outcome of the patient, assessing the risk of cancer recurrence, determining treatment modality, or determining treatment efficacy.

“Having” is an open-ended phrase like “comprising” and “including,” and includes circumstances where additional elements are included and circumstances where they are not.

“Optional” or “optionally” means that the subsequently described event or circumstance may or may not occur, and that the description includes instances where the event or circumstance occurs and instances in which it does not.

As used herein ‘NED’ describes a clinically distinct disease state in which patients show no evidence of disease (NED′) at least 5 years after surgery, ‘PSA’ describes a clinically distinct disease state in which patients show biochemical relapse only (two successive increases in prostate-specific antigen levels but no other symptoms of disease with at least 5 years follow up after surgery; ‘PSA’) and ‘SYS’ describes a clinically distinct disease state in which patients develop biochemical relapse and present with systemic cancer disease or metastases (‘SYS’) within five years after the initial treatment with radical prostatectomy.

The terms “METS”, “SYS”, “systemic event”, “Systemic progression”, “CR” or “Clinical Recurrence” may be used interchangeably and generally refer to patients that experience BCR (biochemical reccurrence) and that develop metastases (confirmed by bone or CT scan). The patients may experience BCR within 5 years of RP (radical prostatectomy). The patients may develop metastases within 5 years of BCR. In some cases, patients regarded as METS may experience BCR after 5 years of RP. In other cases, patients may exhibit cancer recurrence after radical cystectomy (e.g., bladder cancer recurrence). A further preferred surgery is cystectomy. in particular radical or partial cystectomy. The term “cystectomy” refers to removal of all (radical cystectomy) or part (partial cystectomy) of the urinary bladder. This kind of surgery is usually carried out in invasive bladder cancer. and in particular, muscle invasive bladder cancer.

As used herein, the term “about” refers to approximately a +/−10% variation from a given value. It is to be understood that such a variation is always included in any given value provided herein, whether or not it is specifically referred to.

Use of the singular forms “a,” “an,” and “the” include plural references unless the context clearly dictates otherwise. Thus, for example, reference to “a polynucleotide” includes a plurality of polynucleotides, reference to “a target” includes a plurality of such targets, reference to “a normalization method” includes a plurality of such methods, and the like. Additionally, use of specific plural references, such as “two,” “three,” etc., read on larger numbers of the same subject, unless the context clearly dictates otherwise.

Terms such as “connected,” “attached,” “linked” and “conjugated” are used interchangeably herein and encompass direct as well as indirect connection, attachment, linkage or conjugation unless the context clearly dictates otherwise.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values, which are about the same quantity or amount as the recited value, are also within the scope of the invention, as are ranges based thereon. Where a combination is disclosed, each sub-combination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

### Coding and Non-coding Targets

The methods disclosed herein often comprise assaying the expression level of a plurality of targets. The plurality of targets may comprise coding targets and/or non-coding targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets may be differentially expressed. In some instances, a plurality of probe selection regions (PSRs) is differentially expressed.

In some instances, the plurality of targets comprises one or more targets selected from Table 2B, Table 16 or SEQ ID NOs:1-1440. In some instances, the plurality of targets comprises at least about 2, at least about 3, at least about 4, at least about 5, at least about 6, at least about 7, at least about 8, at least about 9, or at least about 10 targets selected from Table 2B, Table 16 or SEQ ID NOs:1-1440. In other instances, the plurality of targets comprises at least about 12, at least about 15, at least about 17, at least about 20, at least about 22, at least about 25, at least about 27, at least about 30, at least about 32, at least about 35, at least about 37, or at least about 40 targets selected from Table 2B, Table 16 or SEQ ID NOs:1-1440. The plurality of targets may comprise about 40, 45, 50, 55, 60, 65, 70, 75, 80, 85, 90, 95, 100 or more targets selected from Table 2B, Table 16 or SEQ ID NOs:1-1440. The plurality of targets may comprise about 110, 120, 130, 140, 150, 160, 170, 180, 190, 200, 225, 250, 275, 300, 325, 350, 375, 400, 425, 450, 475, 500 or more targets selected from Table 2B, Table 16 or SEQ ID NOs:1-1440. The plurality of targets may comprise about 500, 525, 550, 575, 600, 625, 650, 675, 700, 725, 750, 775, 800, 810, 820, 830, 840, 850 or more targets selected from Table 2B, Table 16 or SEQ ID NOs:1-1440. In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

In some instances, the plurality of targets is at least about 70% identical to a sequence selected from SEQ ID NOs 1-1440. Alternatively, the plurality of targets is at least about 80% identical to a sequence selected from SEQ ID NOS 1-1440. In some instances, the plurality of targets is at least about 85% identical to a sequence selected from SEQ ID NOS 1-1440. In some instances, the plurality of targets is at least about 90% identical to a sequence selected from SEQ ID NOS 1-1440. Alternatively, the plurality of targets is at least about 95% identical to a sequence selected from SEQ ID NOS 1-1440.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15 or more targets selected from a Random Forest (RF) classifier. The RF classifier may be an RF2, and RF3, or an RF4 classifier. The RF classifier may be an RF15 classifier (e.g., a Random Forest classifier with 15 targets).

In some instances, the plurality of targets is at least about 70% identical to a sequence selected from a target selected from a RF classifier. Alternatively, the plurality of targets is at least about 80% identical to a sequence selected from a target selected from a RF classifier. In some instances, the plurality of targets is at least about 85% identical to a sequence selected from a target selected from a RF classifier. In some instances, the plurality of targets is at least about 90% identical to a sequence selected from a target selected from a RF classifier. Alternatively, the plurality of targets is at least about 95% identical to a sequence selected from a target selected from a RF classifier. The RF classifier may be an RF2 classifier. The RF classifier may be an RF15 classifier.

The RF15 classifier may comprise SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, or a combination thereof. In some instances, the RF2 classifier is selected from Table 11, Table 12, Table 13, Table 14, Table 15, or Table 20. A RF classifier of the present invention may comprise two or more targets comprising two or more targets selected from Table 2B, Table 16 or SEQ ID NOs:1-1440.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier. The SVM classifier may be an SVM2 classifier.

In some instances, the plurality of targets is at least about 70% identical to a sequence selected from a target selected from a SVM classifier. Alternatively, the plurality of targets is at least about 80% identical to a sequence selected from a target selected from a SVM classifier. In some instances, the plurality of targets is at least about 85% identical to a sequence selected from a target selected from a SVM classifier. In some instances, the plurality of targets is at least about 90% identical to a sequence selected from a target selected from a SVM classifier. Alternatively, the plurality of targets is at least about 95% identical to a sequence selected from a target selected from a SVM classifier. The SVM classifier may be an SVM2 classifier. In some instances, the SVM2 classifier is selected from Table 11, Table 12, Table 13, Table 14, Table 15, or Table 20. A SVM classifier of the present invention may comprise two or more targets comprising two or more targets selected from Table 2B, Table 16 or SEQ ID NOs:1-1440.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100 or more targets selected from a KNN classifier. The plurality of targets may comprise 125, 150, 175, 200, 225, 250, 275, 300, 325, 350, 375, 390 or more targets selected from a KNN classifier. The KNN classifier may be a KNN2 classifier.

In some instances, the plurality of targets is at least about 70% identical to a sequence selected from a target selected from a KNN classifier. Alternatively, the plurality of targets is at least about 80% identical to a sequence selected from a target selected from a KNN classifier. In some instances, the plurality of targets is at least about 85% identical to a sequence selected from a target selected from a KNN classifier. In some instances, the plurality of targets is at least about 90% identical to a sequence selected from a target selected from a KNN classifier. Alternatively, the plurality of targets is at least about 95% identical to a sequence selected from a target selected from a KNN classifier. The KNN classifier may be a KNN2 classifier. In some instances, the KNN2 classifier is selected from Table 11, Table 12, Table 13, Table 14, Table 15, or Table 20. A KNN classifier of the present invention may comprise two or more targets comprising two or more targets selected from Table 2B, Table 16 or SEQ ID NOs:1-1440.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100 or more targets selected from a NB classifier. The plurality of targets may comprise 125, 150, 175, 200, 225, 250, 275, 300, 325, 350, 375, 390 or more targets selected from a NB classifier. The NB classifier may be a NB2 classifier.

In some instances, the plurality of targets is at least about 70% identical to a sequence selected from a target selected from a NB classifier. Alternatively, the plurality of targets is at least about 80% identical to a sequence selected from a target selected from a NB classifier. In some instances, the plurality of targets is at least about 85% identical to a sequence selected from a target selected from a NB classifier. In some instances, the plurality of targets is at least about 90% identical to a sequence selected from a target selected from a NB classifier. Alternatively, the plurality of targets is at least about 95% identical to a sequence selected from a target selected from a NB classifier. The NB classifier may be a NB2 classifier. In some instances, the NB2 classifier is selected from Table 11, Table 12, Table 13, Table 15, or Table 20. An NB classifier of the present invention may comprise two or more targets comprising two or more targets selected from Table 2B, Table 16 or SEQ ID NOs:1-1440.

The plurality of targets may comprise one or more targets selected from a recursive Partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100 or more targets selected from an Rpart classifier. The plurality of targets may comprise 125, 150, 175, 200, 225, 250, 275, 300, 325, 350, 375, 390 or more targets selected from an Rpart classifier. The Rpart classifier may be an Rpart2 classifier.

In some instances, the plurality of targets is at least about 70% identical to a sequence selected from a target selected from an Rpart classifier. Alternatively, the plurality of targets is at least about 80% identical to a sequence selected from a target selected from an Rpart classifier. In some instances, the plurality of targets is at least about 85% identical to a sequence selected from a target selected from an Rpart classifier. In some instances, the plurality of targets is at least about 90% identical to a sequence selected from a target selected from an Rpart classifier. Alternatively, the plurality of targets is at least about 95% identical to a sequence selected from a target selected from an Rpart classifier. The Rpart classifier may be an Rpart2 classifier. In some instances, the Rpart2 classifier is selected from Table 11, Table 12, Table 13, Table 14, Table 15, or Table 20. An Rpart classifier of the present invention may comprise two or more targets comprising two or more targets selected from Table 2B, Table 16 or SEQ ID NOs:1-1440.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15 or more targets selected from a high dimensional discriminate analysis (HDDA) classifier.

In some instances, the plurality of targets is at least about 70% identical to a sequence selected from a target selected from a HDDA classifier. Alternatively, the plurality of targets is at least about 80% identical to a sequence selected from a target selected from a HDDA classifier. In some instances, the plurality of targets is at least about 85% identical to a sequence selected from a target selected from a HDDA classifier. In some instances, the plurality of targets is at least about 90% identical to a sequence selected from a target selected from a HDDA classifier. Alternatively, the plurality of targets is at least about 95% identical to a sequence selected from a target selected from a HDDA classifier.

Probes/Primers

The present invention provides for a probe set for diagnosing, monitoring and/or predicting a status or outcome of a cancer in a subject comprising a plurality of probes, wherein (i) the probes in the set are capable of detecting an expression level of at least one non-coding target; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from bladder cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

One skilled in the art understands that the nucleotide sequence of the polynucleotide probe need not be identical to its target sequence in order to specifically hybridize thereto. The polynucleotide probes of the present invention, therefore, comprise a nucleotide sequence that is at least about 65% identical to a region of the coding target or non-coding target selected from Table 2B, Table 16 or SEQ ID NOs:1-1440. In another embodiment, the nucleotide sequence of the polynucleotide probe is at least about 70% identical a region of the coding target or non-coding target from Table 2B, Table 16 or SEQ ID NOs:1-1440. In another embodiment, the nucleotide sequence of the polynucleotide probe is at least about 75% identical a region of the coding target or non-coding target from Table 2B, Table 16 or SEQ ID NOs:1-1440. In another embodiment, the nucleotide sequence of the polynucleotide probe is at least about 80% identical a region of the coding target or non-coding target from Table 2B, Table 16 or SEQ ID NOs:1-1440. In another embodiment, the nucleotide sequence of the polynucleotide probe is at least about 85% identical a region of the coding target or non-coding target from Table 2B, Table 16 or SEQ ID NOs:1-1440. In another embodiment, the nucleotide sequence of the polynucleotide probe is at least about 90% identical a region of the coding target or non-coding target from Table 2B, Table 16 or SEQ ID NOs:1-1440. In a further embodiment, the nucleotide sequence of the polynucleotide probe is at least about 95% identical to a region of the coding target or non-coding target from Table 2B, Table 16 or SEQ ID NOs:1-1440.

Methods of determining sequence identity are known in the art and can be determined, for example, by using the BLASTN program of the University of Wisconsin Computer Group (GCG) software or provided on the NCB′ website. The nucleotide sequence of the polynucleotide probes of the present invention may exhibit variability by differing (e.g. by nucleotide substitution, including transition or transversion) at one, two, three, four or more nucleotides from the sequence of the coding target or non-coding target.

Other criteria known in the art may be employed in the design of the polynucleotide probes of the present invention. For example, the probes can be designed to have <50% G content. The probes can be designed to have between about 25% and about 70% G+C content. Strategies to optimize probe hybridization to the target nucleic acid sequence can also be included in the process of probe selection.

Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets selected from Table 2B, Table 16 or SEQ ID NOs:1-1440. Alternatively, the probe set comprise a plurality of target sequences that hybridize to at least about 10 coding targets and/or non-coding targets selected from Table 2B, Table 16 or SEQ ID NOs:1-1440. In some embodiments, the probe set comprise a plurality of target sequences that hybridize to at least about 15 coding targets and/or non-coding targets selected from Table 2B, Table 16 or SEQ ID NOs:1-1440. In some embodiments, the probe set comprise a plurality of target sequences that hybridize to at least about 20 coding targets and/or non-coding targets selected from Table 2B, Table 16 or SEQ ID NOs:1-1440. In some embodiments, the probe set comprise a plurality of target sequences that hybridize to at least about 30 coding targets and/or non-coding targets selected from Table 2B, Table 16 or SEQ ID NOs:1-1440. The probe set can comprise a plurality of targets that hybridize to at least about 40, 50, 60, 70, 80, 90, 100 or more coding targets and/or non-coding targets selected from Table 2B, Table 16 or SEQ ID NOs:1-1440. The probe set can comprise a plurality of targets that hybridize to at least about 100, 125, 150, 175, 200, 225, 250, 275, 300 or more coding targets and/or non-coding targets selected from Table 2B, Table 16 or SEQ ID NOs:1-1440. The probe set can comprise a plurality of targets that hybridize to at least about 300, 325, 350, 375, 400, 425, 450, 475, 500, 525, 550, 575, 600 or more coding targets and/or non-coding targets selected from Table 2B, Table 16 or SEQ ID NOs:1-1440. The probe set can comprise a plurality of targets that hybridize to at least about 600, 625, 650, 675, 700, 725, 750, 775, 800, 825, 850 or more coding targets and/or non-coding targets selected from Table 2B, Table 16 or SEQ ID NOs:1-1440.

In some embodiments, the probe set comprises a plurality of target sequences that hybridize to a plurality of targets, wherein the at least about 20% of the plurality of targets are targets selected from Table 2B, Table 16 or SEQ ID NOs:1-1440. In some embodiments, the probe set comprises a plurality of target sequences that hybridize to a plurality of targets, wherein the at least about 25% of the plurality of targets are targets selected from Table 2B, Table 16 or SEQ ID NOs:1-1440. In some embodiments, the probe set comprise a plurality of target sequences that hybridize to a plurality of targets, wherein the at least about 30% of the plurality of targets are targets selected from Table 2B, Table 16 or SEQ ID NOs:1-1440. In some embodiments, the probe set comprise a plurality of target sequences that hybridize to a plurality of targets, wherein the at least about 35% of the plurality of targets are targets selected from Table 2B, Table 16 or SEQ ID NOs:1-1440. In some embodiments, the probe set comprise a plurality of target sequences that hybridize to a plurality of targets, wherein the at least about 40% of the plurality of targets are targets selected from Table 2B, Table 16 or SEQ ID NOs:1-1440. In some embodiments, the probe set comprise a plurality of target sequences that hybridize to a plurality of targets, wherein the at least about 45% of the plurality of targets are targets selected from Table 2B, Table 16 or SEQ ID NOs:1-1440. In some embodiments, the probe set comprise a plurality of target sequences that hybridize to a plurality of targets, wherein the at least about 50% of the plurality of targets are targets selected from Table 2B, Table 16 or SEQ ID NOs:1-1440. In some embodiments, the probe set comprise a plurality of target sequences that hybridize to a plurality of targets, wherein the at least about 60% of the plurality of targets are targets selected from Table 2B, Table 16 or SEQ ID NOs:1-1440. In some embodiments, the probe set comprise a plurality of target sequences that hybridize to a plurality of targets, wherein the at least about 70% of the plurality of targets are targets selected from Table 2B, Table 16 or SEQ ID NOs:1-1440.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target selected from Table 2B, Table 16 or SEQ ID NOs:1-1440 (or subgroups thereof as set forth herein), an RNA form thereof, or a complement to either thereof.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of polynucleotide probes or primers useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide probes or primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridization with an appropriate nucleic acid target compound. An example of such an oligonucleotide mimetic, which has been shown to have excellent hybridization properties, is a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates polynucleotide probes or primers comprising “locked nucleic acids” (LNAs), which may be novel conformationally restricted oligonucleotide analogues containing a methylene bridge that connects the 2′-O of ribose with the 4′-C. LNA and LNA analogues may display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described. Studies of mismatched sequences show that LNA obey the Watson-Crick base pairing rules with generally improved selectivity compared to the corresponding unmodified reference strands.

LNAs may form duplexes with complementary DNA or RNA or with complementary LNA, with high thermal affinities. The universality of LNA-mediated hybridization has been emphasized by the formation of exceedingly stable LNA:LNA duplexes. LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA and 2′-methylamino-LNA has been described and thermal stability of their duplexes with complementary RNA and DNA strands reported. Preparation of phosphorothioate-LNA and 2′-thio-LNA have also been described.

Modified polynucleotide probes or primers may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C2 to C₁₀ alkenyl and alkynyl. Examples of such groups are: O[(CH₂)ₙO]ₘCH₃, O(CH₂)ₙ OCH₃, O(CH₂)ₙ NH₂, O(CH₂)ₙ CH₃ ONH₂, and O(CH₂)ₙ, ON[((CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂ CH₂ OCH₃, also known as 2′-O-(2-methoxyethyl) or 2′-MOE), 2′-dimethylaminooxyethoxy (O(CH2)2 ON(CH₃)₂ group, also known as 2′-DMA0E), 2′-methoxy (2′-O-CH₃), 2′-aminopropoxy (2′-OCH₂ CH₂ CH₂ NH₂) and 2′-fluoro (2′-F).

Similar modifications may also be made at other positions on the polynucleotide probes or primers, particularly the 3′ position of the sugar on the 3′ terminal nucleotide or in 2¹-5′ linked oligonucleotides and the 5′ position of 5′ terminal nucleotide. Polynucleotide probes or primers may also have sugar mimetics such as cyclobutyl moieties in place of the pentofuranosyl sugar.

Polynucleotide probes or primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C.

One skilled in the art recognizes that it is not necessary for all positions in a given polynucleotide probe or primer to be uniformly modified. The present invention, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences which are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

The polynucleotide probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target sequence is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the invention. A variety of different coding schemes are known in the art, including fluorophores, including SCNCs, deposited metals, and RF tags.

Polynucleotides from the described target sequences may be employed as probes for detecting target sequences expression, for ligation amplification schemes, or may be used as primers for amplification schemes of all or a portion of a target sequences. When amplified, either strand produced by amplification may be provided in purified and/or isolated form.

In one embodiment, polynucleotides of the invention include (a) a nucleic acid depicted in Table 1; (b) an RNA form of any one of the nucleic acids depicted in Table 1; (c) a peptide nucleic acid form of any of the nucleic acids depicted in Table 1; (d) a nucleic acid comprising at least 20 consecutive bases of any of (a-c); (e) a nucleic acid comprising at least 25 bases having at least 90% sequenced identity to any of (a-c); and (f) a complement to any of (a-e).

Complements may take any polymeric form capable of base pairing to the species recited in (a)-(e), including nucleic acid such as RNA or DNA, or may be a neutral polymer such as a peptide nucleic acid. Polynucleotides of the invention can be selected from the subsets of the recited nucleic acids described herein, as well as their complements.

In some embodiments, polynucleotides of the invention comprise at least 20 consecutive bases of the nucleic acid sequence of a target selected from Table 2B, Table 16 or SEQ ID NOs: 1-1440 or a complement thereto. The polynucleotides may comprise at least 21, 22, 23, 24, 25, 27, 30, 32, 35 or more consecutive bases of the nucleic acids sequence of a target selected from Table 1, as applicable.

The polynucleotides may be provided in a variety of formats, including as solids, in solution, or in an array. The polynucleotides may optionally comprise one or more labels, which may be chemically and/or enzymatically incorporated into the polynucleotide.

In one embodiment, solutions comprising polynucleotide and a solvent are also provided. In some embodiments, the solvent may be water or may be predominantly aqueous. In some embodiments, the solution may comprise at least two, three, four, five, six, seven, eight, nine, ten, twelve, fifteen, seventeen, twenty or more different polynucleotides, including primers and primer pairs, of the invention. Additional substances may be included in the solution, alone or in combination, including one or more labels, additional solvents, buffers, biomolecules, polynucleotides, and one or more enzymes useful for performing methods described herein, including polymerases and ligases. The solution may further comprise a primer or primer pair capable of amplifying a polynucleotide of the invention present in the solution.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like, and include semiconductor nanocrystals.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

Silica aerogels can also be used as substrates, and can be prepared by methods known in the art. Aerogel substrates may be used as free standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. The surface can be optically transparent and can have surface Si—OH functionalities, such as those found on silica surfaces.

The substrate and/or its optional surface can be chosen to provide appropriate characteristics for the synthetic and/or detection methods used. The substrate and/or surface can be transparent to allow the exposure of the substrate by light applied from multiple directions. The substrate and/or surface may be provided with reflective “mirror” structures to increase the recovery of light.

The substrate and/or its surface is generally resistant to, or is treated to resist, the conditions to which it is to be exposed in use, and can be optionally treated to remove any resistant material after exposure to such conditions.

The substrate or a region thereof may be encoded so that the identity of the sensor located in the substrate or region being queried may be determined. Any suitable coding scheme can be used, for example optical codes, RFID tags, magnetic codes, physical codes, fluorescent codes, and combinations of codes.

### Preparation of Probes and Primers

The polynucleotide probes or primers of the present invention can be prepared by conventional techniques well-known to those skilled in the art. For example, the polynucleotide probes can be prepared using solid-phase synthesis using commercially available equipment. As is well-known in the art, modified oligonucleotides can also be readily prepared by similar methods. The polynucleotide probes can also be synthesized directly on a solid support according to methods standard in the art. This method of synthesizing polynucleotides is particularly useful when the polynucleotide probes are part of a nucleic acid array.

Polynucleotide probes or primers can be fabricated on or attached to the substrate by any suitable method, for example the methods described in U.S. Pat. No. 5,143,854, PCT Publ. No. WO 92/10092, U.S. patent application Ser. No. 07/624,120, filed Dec. 6, 1990 (now abandoned), Fodor et al., Science, 251: 767-777 (1991), and PCT Publ. No. WO 90/15070). Techniques for the synthesis of these arrays using mechanical synthesis strategies are described in, e.g., PCT Publication No. WO 93/09668 and U.S. Pat. No. 5,384,261. Still further techniques include bead based techniques such as those described in PCT Appl. No. PCT/US93/04145 and pin based methods such as those described in U.S. Pat. No. 5,288,514. Additional flow channel or spotting methods applicable to attachment of sensor polynucleotides to a substrate are described in U.S. patent application Ser. No. 07/980,523, filed Nov. 20, 1992, and U.S. Pat. No. 5,384,261.

Alternatively, the polynucleotide probes of the present invention can be prepared by enzymatic digestion of the naturally occurring target gene, or mRNA or cDNA derived therefrom, by methods known in the art.

### Diagnostic Samples

Diagnostic samples for use with the systems and in the methods of the present invention comprise nucleic acids suitable for providing RNAs expression information. In principle, the biological sample from which the expressed RNA is obtained and analyzed for target sequence expression can be any material suspected of comprising cancer tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100 or more primers or pairs of primers suitable for selectively amplifying one or more targets. The kit may comprise at least 100, 125, 150, 175, 200, 250, 300, 350, 400, 450, 500 or more primers or pairs of primers suitable for selectively amplifying one or more targets. The kit may comprise at least 500, 550, 600, 650, 700, 750, 800, 850 or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, at least a portion of a nucleic acid sequence depicted in one of SEQ ID NOs: 1-1440, a nucleic acid sequence corresponding to a target selected from Table 1, an RNA form thereof, or a complement to either thereof. The kit may include a plurality of such primers or primer pairs which can specifically amplify a corresponding plurality of different amplify a non-coding target, coding target, exonic, or non-exonic transcript described herein, nucleic acids depicted in one of SEQ ID NOs: 1-1440, a nucleic acid sequence corresponding to a target selected from Table 1, RNA forms thereof, or complements thereto. At least two, three, four or five primers or pairs of primers suitable for selectively amplifying the one or or targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Devices

Devices useful for performing methods of the invention are also provided. The devices can comprise means for characterizing the expression level of a target sequence of the invention, for example components for performing one or more methods of nucleic acid extraction, amplification, and/or detection. Such components may include one or more of an amplification chamber (for example a thermal cycler), a plate reader, a spectrophotometer, capillary electrophoresis apparatus, a chip reader, and or robotic sample handling components. These components ultimately can obtain data that reflects the expression level of the target sequences used in the assay being employed.

The devices may include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Exemplary excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices typically comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory that contains a sample designation and a record of expression levels obtained from the sample. Linkage of the results to the sample can also include a linkage to a particular sample receptacle in the device, which is also linked to the sample identity.

In some instances, the devices also comprise a means for correlating the expression levels of the target sequences being studied with a prognosis of disease outcome. In some instances, such means comprises one or more of a variety of correlative techniques, including lookup tables, algorithms, multivariate models, and linear or nonlinear combinations of expression models or algorithms. The expression levels may be converted to one or more likelihood scores, reflecting likelihood that the patient providing the sample may exhibit a particular disease outcome. The models and/or algorithms can be provided in machine readable format and can optionally further designate a treatment modality for a patient or class of patients.

The device also comprises output means for outputting the disease status, prognosis and/or a treatment modality. Such output means can take any form which transmits the results to a patient and/or a healthcare provider, and may include a monitor, a printed format, or both. The device may use a computer system for performing one or more of the steps provided.

In some embodiments, the method, systems, and kits disclosed herein further comprise the transmission of data/information. For example, data/information derived from the detection and/or quantification of the target may be transmitted to another device and/or instrument. In some instances, the information obtained from an algorithm is transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc).

In some instances, transmission of the data/information comprises digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. In some embodiments, the data is represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation′m FFPE Exon Module, WT-Ovation™ FFPE Exon Module RiboAmp and RiboAmp ^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert(R) Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the invention. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxam-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

### TaqMan® Gene Expression Analysis

TaqMan®RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 2B, Table 16 or SEQ ID NOs: 1-1440 or a product derived thereof can be used. Desirably, an array may be specific for 5, 10, 15, 20, 25, 30, 50, 75, 100, 150, 200 or more of transcripts of a target selected from Table 2B, Table 16 or SEQ ID NOs:1-1440. The array may be specific for 200, 225, 250, 275, 300, 325, 350, 375, 400 or more of the transcripts of a target selected from Table 2B, Table 16 or SEQ ID NOs:1-1440. The array may be specific for 400, 425, 450, 475, 500, 525, 550, 575, 600 or more of the transcripts of a target selected from Table 2B, Table 16 or SEQ ID NOs:1-1440. The array may be specific for 600, 625, 650, 675, 700, 725, 750, 775, 800, 825, 850 or more of the transcripts of a target selected from Table 2B, Table 16 or SEQ ID NOs:1-1440. Expression of these sequences may be detected alone or in combination with other transcripts. In some embodiments, an array is used which comprises a wide range of sensor probes for bladder-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognosticarray is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, frozen robust multichip average (fRMA), Single Channel Array Normalization (SCAN), ComBat (Combining Batches of gene expression), Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Additional Techniques and Tests

Factors known in the art for diagnosing and/or suggesting, selecting, designating, recommending or otherwise determining a course of treatment for a patient or class of patients suspected of having cancer can be employed in combination with measurements of the target sequence expression. The methods disclosed herein may include additional techniques such as cytology, histology, ultrasound analysis, MRI results, CT scan results, and measurements of PSA levels.

Certified tests for classifying disease status and/or designating treatment modalities may also be used in diagnosing, predicting, and/or monitoring the status or outcome of a cancer in a subject. A certified test may comprise a means for characterizing the expression levels of one or more of the target sequences of interest, and a certification from a government regulatory agency endorsing use of the test for classifying the disease status of a biological sample.

In some embodiments, the certified test may comprise reagents for amplification reactions used to detect and/or quantitate expression of the target sequences to be characterized in the test. An array of probe nucleic acids can be used, with or without prior target amplification, for use in measuring target sequence expression.

The test is submitted to an agency having authority to certify the test for use in distinguishing disease status and/or outcome. Results of detection of expression levels of the target sequences used in the test and correlation with disease status and/or outcome are submitted to the agency. A certification authorizing the diagnostic and/or prognostic use of the test is obtained.

Also provided are portfolios of expression levels comprising a plurality of normalized expression levels of the target selected from Table 2B, Table 16 or SEQ ID NOs:1-1440. Such portfolios may be provided by performing the methods described herein to obtain expression levels from an individual patient or from a group of patients. The expression levels can be normalized by any method known in the art; exemplary normalization methods that can be used in various embodiments include Robust Multichip Average (RMA), frozen robust multichip average (fRMA), Single Channel Array Normalization (SCAN), ComBat (Combining Batches of gene expression), probe logarithmic intensity error estimation (PLIER), non-linear fit (NLFIT) quantile-based and nonlinear normalization, and combinations thereof. Background correction can also be performed on the expression data; exemplary techniques useful for background correction include mode of intensities, normalized using median polish probe modeling and sketch-normalization.

In some embodiments, portfolios are established such that the combination of genes in the portfolio exhibit improved sensitivity and specificity relative to known methods. In considering a group of genes for inclusion in a portfolio, a small standard deviation in expression measurements correlates with greater specificity. Other measurements of variation such as correlation coefficients can also be used in this capacity. The invention also encompasses the above methods where the expression level determines the status or outcome of a cancer in the subject with at least about 45% specificity. In some embodiments, the expression level determines the status or outcome of a cancer in the subject with at least about 50% specificity. In some embodiments, the expression level determines the status or outcome of a cancer in the subject with at least about 55% specificity. In some embodiments, the expression level determines the status or outcome of a cancer in the subject with at least about 60% specificity. In some embodiments, the expression level determines the status or outcome of a cancer in the subject with at least about 65% specificity. In some embodiments, the expression level determines the status or outcome of a cancer in the subject with at least about 70% specificity. In some embodiments, the expression level determines the status or outcome of a cancer in the subject with at least about 75% specificity. In some embodiments, the expression level determines the status or outcome of a cancer in the subject with at least about 80% specificity. In some embodiments, t the expression level determines the status or outcome of a cancer in the subject with at least about 85% specificity. In some embodiments, the expression level determines the status or outcome of a cancer in the subject with at least about 90% specificity. In some embodiments, the expression level determines the status or outcome of a cancer in the subject with at least about 95% specificity.

The invention also encompasses the any of the methods disclosed herein where the accuracy of diagnosing, monitoring, and/or predicting a status or outcome of a cancer is at least about 45%. In some embodiments, the accuracy of diagnosing, monitoring, and/or predicting a status or outcome of a cancer is at least about 50%. In some embodiments, the accuracy of diagnosing, monitoring, and/or predicting a status or outcome of a cancer is at least about 55%. In some embodiments, the accuracy of diagnosing, monitoring, and/or predicting a status or outcome of a cancer is at least about 60%. In some embodiments, the accuracy of diagnosing, monitoring, and/or predicting a status or outcome of a cancer is at least about 65%. In some embodiments, the accuracy of diagnosing, monitoring, and/or predicting a status or outcome of a cancer is at least about 70%. In some embodiments, the accuracy of diagnosing, monitoring, and/or predicting a status or outcome of a cancer is at least about 75%. In some embodiments, the accuracy of diagnosing, monitoring, and/or predicting a status or outcome of a cancer is at least about 80%. In some embodiments, the accuracy of diagnosing, monitoring, and/or predicting a status or outcome of a cancer is at least about 85%. In some embodiments, the accuracy of diagnosing, monitoring, and/or predicting a status or outcome of a cancer is at least about 90%. In some embodiments, the accuracy of diagnosing, monitoring, and/or predicting a status or outcome of a cancer is at least about 95%.

The accuracy of a classifier or biomarker may be determined by the 95% confidence interval (CI). Generally, a classifier or biomarker is considered to have good accuracy if the 95% CI does not overlap 1. In some instances, the 95% CI of a classifier or biomarker is at least about 1.08, 1.10, 1.12, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, or 1.35 or more. The 95% CI of a classifier or biomarker may be at least about 1.14, 1.15, 1.16, 1.20, 1.21, 1.26, or 1.28. The 95% CI of a classifier or biomarker may be less than about 1.75, 1.74, 1.73, 1.72, 1.71, 1.70, 1.69, 1.68, 1.67, 1.66, 1.65, 1.64, 1.63, 1.62, 1.61, 1.60, 1.59, 1.58, 1.57, 1.56, 1.55, 1.54, 1.53, 1.52, 1.51, 1.50 or less. The 95% CI of a classifier or biomarker may be less than about 1.61, 1.60, 1.59, 1.58, 1.56, 1.55, or 1.53. The 95% CI of a classifier or biomarker may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some instances, the accuracy of a biomarker or classifier is dependent on the difference in range of the 95% CI (e.g., difference in the high value and low value of the 95% CI interval). Generally, biomarkers or classifiers with large differences in the range of the 95% CI interval have greater variability and are considered less accurate than biomarkers or classifiers with small differences in the range of the 95% CI intervals. In some instances, a biomarker or classifier is considered more accurate if the difference in the range of the 95% CI is less than about 0.60, 0.55, 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25 or less. The difference in the range of the 95% CI of a biomarker or classifier may be less than about 0.48, 0.45, 0.44, 0.42, 0.40, 0.37, 0.35, 0.33, or 0.32. In some instances, the difference in the range of the 95% CI for a biomarker or classifier is between about 0.25 to about 0.50, between about 0.27 to about 0.47, or between about 0.30 to about 0.45.

The invention also encompasses the any of the methods disclosed herein where the sensitivity is at least about 45%. In some embodiments, the sensitivity is at least about 50%. In some embodiments, the sensitivity is at least about 55%. In some embodiments, the sensitivity is at least about 60%. In some embodiments, the sensitivity is at least about 65%. In some embodiments, the sensitivity is at least about 70%. In some embodiments, the sensitivity is at least about 75%. In some embodiments, the sensitivity is at least about 80%. In some embodiments, the sensitivity is at least about 85%. In some embodiments, the sensitivity is at least about 90%. In some embodiments, the sensitivity is at least about 95%.

In some instances, the classifiers or biomarkers disclosed herein are clinically significant. In some instances, the clinical significance of the classifiers or biomarkers is determined by the AUC value. In order to be clinically significant, the AUC value is at least about 0.5, 0.55, 0.6, 0.65, 0.7, 0.75, 0.8, 0.85, 0.9, or 0.95. The clinical significance of the classifiers or biomarkers can be determined by the percent accuracy. For example, a classifier or biomarker is determined to be clinically significant if the accuracy of the classifier or biomarker is at least about 50%, 55%, 60%, 65%, 70%, 72%, 75%, 77%, 80%, 82%, 84%, 86%, 88%, 90%, 92%, 94%, 96%, or 98%. In other instances, the clinical significance of the classifiers or biomarkers is determined by the median fold difference (MDF) value. In order to be clinically significant, the MDF value is at least about 0.8, 0.9, 1.0, 1.1, 1.2, 1.3, 1.4, 1.5, 1.6, 1.7, 1.9, or 2.0. In some instances, the MDF value is greater than or equal to 1.1. In other instances, the MDF value is greater than or equal to 1.2. Alternatively, or additionally, the clinical significance of the classifiers or biomarkers is determined by the t-test P-value. In some instances, in order to be clinically significant, the t-test P-value is less than about 0.070, 0.065, 0.060, 0.055, 0.050, 0.045, 0.040, 0.035, 0.030, 0.025, 0.020, 0.015, 0.010, 0.005, 0.004, or 0.003. The t-test P-value can be less than about 0.050. Alternatively, the t-test P-value is less than about 0.010. In some instances, the clinical significance of the classifiers or biomarkers is determined by the clinical outcome. For example, different clinical outcomes can have different minimum or maximum thresholds for AUC values, MDF values, t-test P-values, and accuracy values that would determine whether the classifier or biomarker is clinically significant. In another example, a classifier or biomarker is considered clinically significant if the P-value of the t-test was less than about 0.08, 0.07, 0.06, 0.05, 0.04, 0.03, 0.02, 0.01, 0.005, 0.004, 0.003, 0.002, or 0.001. In some instances, the P-value may be based on any of the following comparisons: BCR vs non-BCR, CP vs non-CP, PCSM vs non-PCSM. For example, a classifier or biomarker is determined to be clinically significant if the P-values of the differences between the KM curves for BCR vs non-BCR, CP vs non-CP, PCSM vs non-PCSM is lower than about 0.08, 0.07, 0.06, 0.05, 0.04, 0.03, 0.02, 0.01, 0.005, 0.004, 0.003, 0.002, or 0.001.

In some instances, the performance of the classifier or biomarker is based on the odds ratio. A classifier or biomarker may be considered to have good performance if the odds ratio is at least about 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.52, 1.55, 1.57, 1.60, 1.62, 1.65, 1.67, 1.70 or more. In some instances, the odds ratio of a classifier or biomarker is at least about 1.33.

The clinical significance of the classifiers and/or biomarkers may be based on Univariable Analysis Odds Ratio P-value (uvaORPval). The Univariable Analysis Odds Ratio P-value (uvaORPval) of the classifier and/or biomarker may be between about 0-0.4. The Univariable Analysis Odds Ratio P-value (uvaORPval) of the classifier and/or biomarker may be between about 0-0.3. The Univariable Analysis Odds Ratio P-value (uvaORPval) of the classifier and/or biomarker may be between about 0-0.2. The Univariable Analysis Odds Ratio P-value (uvaORPval) of the classifier and/or biomarker may be less than or equal to 0.25, 0.22, 0.21, 0.20, 0.19, 0.18, 0.17, 0.16, 0.15, 0.14, 0.13, 0.12, 0.11. The Univariable Analysis Odds Ratio P-value (uvaORPval) of the classifier and/or biomarker may be less than or equal to 0.10, 0.09, 0.08, 0.07, 0.06, 0.05, 0.04, 0.03, 0.02, 0.01. The Univariable Analysis Odds Ratio P-value (uvaORPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on multivariable analysis Odds Ratio P-value (mvaORPval). The multivariable analysis Odds Ratio P-value (mvaORPval) of the classifier and/or biomarker may be between about 0-1. The multivariable analysis Odds Ratio P-value (mvaORPval) of the classifier and/or biomarker may be between about 0-0.9. The multivariable analysis Odds Ratio P-value (mvaORPval) of the classifier and/or biomarker may be between about 0-0.8. The multivariable analysis Odds Ratio P-value (mvaORPval) of the classifier and/or biomarker may be less than or equal to 0.90, 0.88, 0.86, 0.84, 0.82, 0.80. The multivariable analysis Odds Ratio P-value (mvaORPval) of the classifier and/or biomarker may be less than or equal to 0.78, 0.76, 0.74, 0.72, 0.70, 0.68, 0.66, 0.64, 0.62, 0.60, 0.58, 0.56, 0.54, 0.52, 0.50. The multivariable analysis Odds Ratio P-value (mvaORPval) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The multivariable analysis Odds Ratio P-value (mvaORPval) of the classifier and/or biomarker may be less than or equal to 0.10, 0.09, 0.08, 0.07, 0.06, 0.05, 0.04, 0.03, 0.02, 0.01. The multivariable analysis Odds Ratio P-value (mvaORPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Kaplan Meier P-value (KM P-value). The Kaplan Meier P-value (KM P-value) of the classifier and/or biomarker may be between about 0-0.8. The Kaplan Meier P-value (KM P-value) of the classifier and/or biomarker may be between about 0-0.7. The Kaplan Meier P-value (KM P-value) of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Kaplan Meier P-value (KM P-value) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Kaplan Meier P-value (KM P-value) of the classifier and/or biomarker may be less than or equal to 0.10, 0.09, 0.08, 0.07, 0.06, 0.05, 0.04, 0.03, 0.02, 0.01. The Kaplan Meier P-value (KM P-value) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the survival AUC value (survAUC). The survival AUC value (survAUC) of the classifier and/or biomarker may be between about 0-1. The survival AUC value (survAUC) of the classifier and/or biomarker may be between about 0-0.9. The survival AUC value (survAUC) of the classifier and/or biomarker may be less than or equal to 1, 0.98, 0.96, 0.94, 0.92, 0.90, 0.88, 0.86, 0.84, 0.82, 0.80. The survival AUC value (survAUC) of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The survival AUC value (survAUC) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The survival AUC value (survAUC) of the classifier and/or biomarker may be less than or equal to 0.10, 0.09, 0.08, 0.07, 0.06, 0.05, 0.04, 0.03, 0.02, 0.01. The survival AUC value (survAUC) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Univariable Analysis Hazard Ratio P-value (uvaHRPval). The Univariable Analysis Hazard Ratio P-value (uvaHRPval) of the classifier and/or biomarker may be between about 0-0.4. The Univariable Analysis Hazard Ratio P-value (uvaHRPval) of the classifier and/or biomarker may be between about 0-0.3. The Univariable Analysis Hazard Ratio P-value (uvaHRPval) of the classifier and/or biomarker may be less than or equal to 0.40, 0.38, 0.36, 0.34, 0.32. The Univariable Analysis Hazard Ratio P-value (uvaHRPval) of the classifier and/or biomarker may be less than or equal to 0.30, 0.29, 0.28, 0.27, 0.26, 0.25, 0.24, 0.23, 0.22, 0.21, 0.20. The Univariable Analysis Hazard Ratio P-value (uvaHRPval) of the classifier and/or biomarker may be less than or equal to 0.19, 0.18, 0.17, 0.16, 0.15, 0.14, 0.13, 0.12, 0.11. The Univariable Analysis Hazard Ratio P-value (uvaHRPval) of the classifier and/or biomarker may be less than or equal to 0.10, 0.09, 0.08, 0.07, 0.06, 0.05, 0.04, 0.03, 0.02, 0.01. The Univariable Analysis Hazard Ratio P-value (uvaHRPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Multivariable Analysis Hazard Ratio P-value (mvaHRPval). The Multivariable Analysis Hazard Ratio P-value (mvaHRPval) of the classifier and/or biomarker may be between about 0-1. The Multivariable Analysis Hazard Ratio P-value (mvaHRPval) of the classifier and/or biomarker may be between about 0-0.9. The Multivariable Analysis Hazard Ratio P-value (mvaHRPval) of the classifier and/or biomarker may be less than or equal to 1, 0.98, 0.96, 0.94, 0.92, 0.90, 0.88, 0.86, 0.84, 0.82, 0.80. The Multivariable Analysis Hazard Ratio P-value (mvaHRPval) of the classifier and/or biomarker may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Multivariable Analysis Hazard Ratio P-value (mvaHRPval) of the classifier and/or biomarker may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Multivariable Analysis Hazard Ratio P-value (mvaHRPval) of the classifier and/or biomarker may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier and/or biomarker may be less than or equal to 0.009, 0.008, 0.007, 0.006, 0.005, 0.004, 0.003, 0.002, 0.001.

The clinical significance of the classifiers and/or biomarkers may be based on the Multivariable Analysis Hazard Ratio P-value (mvaHRPval). The Multivariable Analysis Hazard Ratio P-value (mvaHRPval) of the classifier and/or biomarker may be between about 0 to about 0.60. significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be between about 0 to about 0.50. significance of the classifier and/or biomarker may be based on the Multivariable Analysis Hazard Ratio P-value (mvaHRPval). The Multivariable Analysis Hazard Ratio P-value (mvaHRPval) of the classifier and/or biomarker may be less than or equal to 0.50, 0.47, 0.45, 0.43, 0.40, 0.38, 0.35, 0.33, 0.30, 0.28, 0.25, 0.22, 0.20, 0.18, 0.16, 0.15, 0.14, 0.13, 0.12, 0.11, 0.10. The Multivariable Analysis Hazard Ratio P-value (mvaHRPval) of the classifier and/or biomarker may be less than or equal to 0.10, 0.09, 0.08, 0.07, 0.06, 0.05, 0.04, 0.03, 0.02, 0.01. The Multivariable Analysis Hazard Ratio P-value (mvaHRPval) of the classifier and/or biomarker may be less than or equal to 0.01, 0.009, 0.008, 0.007, 0.006, 0.005, 0.004, 0.003, 0.002, 0.001.

The classifiers and/or biomarkers disclosed herein may outperform current classifiers or clinical variables in providing clinically relevant analysis of a sample from a subject. In some instances, the classifiers or biomarkers may more accurately predict a clinical outcome or status as compared to current classifiers or clinical variables. For example, a classifier or biomarker may more accurately predict metastatic disease. Alternatively, a classifier or biomarker may more accurately predict no evidence of disease. In some instances, the classifier or biomarker may more accurately predict death from a disease. The performance of a classifier or biomarker disclosed herein may be based on the AUC value, odds ratio, 95% CI, difference in range of the 95% CI, p-value or any combination thereof.

The performance of the classifiers and/or biomarkers disclosed herein may be determined by AUC values and an improvement in performance may be determined by the difference in the AUC value of the classifier or biomarker disclosed herein and the AUC value of current classifiers or clinical variables. In some instances, a classifier and/or biomarker disclosed herein outperforms current classifiers or clinical variables when the AUC value of the classifier and/or or biomarker disclosed herein is greater than the AUC value of the current classifiers or clinical variables by at least about 0.05, 0.06, 0.07, 0.08, 0.09, 0.10, 0.11, 0.12, 0.13, 0.14, 0.15, 0.16, 0.17, 0.18, 0.19, 0.20, 0.022, 0.25, 0.27, 0.30, 0.32, 0.35, 0.37, 0.40, 0.42, 0.45, 0.47, 0.50 or more. In some instances, the AUC value of the classifier and/or or biomarker disclosed herein is greater than the AUC value of the current classifiers or clinical variables by at least about 0.10. In some instances, the AUC value of the classifier and/or or biomarker disclosed herein is greater than the AUC value of the current classifiers or clinical variables by at least about 0.13. In some instances, the AUC value of the classifier and/or or biomarker disclosed herein is greater than the AUC value of the current classifiers or clinical variables by at least about 0.18.

The performance of the classifiers and/or biomarkers disclosed herein may be determined by the odds ratios and an improvement in performance may be determined by comparing the odds ratio of the classifier or biomarker disclosed herein and the odds ratio of current classifiers or clinical variables. Comparison of the performance of two or more classifiers, biomarkers, and/or clinical variables can be generally be based on the comparison of the absolute value of (1-odds ratio) of a first classifier, biomarker or clinical variable to the absolute value of (1-odds ratio) of a second classifier, biomarker or clinical variable. Generally, the classifier, biomarker or clinical variable with the greater absolute value of (1-odds ratio) can be considered to have better performance as compared to the classifier, biomarker or clinical variable with a smaller absolute value of (1-odds ratio).

In some instances, the performance of a classifier, biomarker or clinical variable is based on the comparison of the odds ratio and the 95% confidence interval (CI). For example, a first classifier, biomarker or clinical variable may have a greater absolute value of (1-odds ratio) than a second classifier, biomarker or clinical variable, however, the 95% CI of the first classifier, biomarker or clinical variable may overlap 1 (e.g., poor accuracy), whereas the 95% CI of the second classifier, biomarker or clinical variable does not overlap 1. In this instance, the second classifier, biomarker or clinical variable is considered to outperform the first classifier, biomarker or clinical variable because the accuracy of the first classifier, biomarker or clinical variable is less than the accuracy of the second classifier, biomarker or clinical variable. In another example, a first classifier, biomarker or clinical variable may outperform a second classifier, biomarker or clinical variable based on a comparison of the odds ratio; however, the difference in the 95% CI of the first classifier, biomarker or clinical variable is at least about 2 times greater than the 95% CI of the second classifier, biomarker or clinical variable. In this instance, the second classifier, biomarker or clinical variable is considered to outperform the first classifier.

In some instances, a classifier or biomarker disclosed herein more accurate than a current classifier or clinical variable. The classifier or biomarker disclosed herein is more accurate than a current classifier or clinical variable if the range of 95% CI of the classifier or biomarker disclosed herein does not span or overlap 1 and the range of the 95% CI of the current classifier or clinical variable spans or overlaps 1.

In some instances, a classifier or biomarker disclosed herein more accurate than a current classifier or clinical variable. The classifier or biomarker disclosed herein is more accurate than a current classifier or clinical variable when difference in range of the 95% CI of the classifier or biomarker disclosed herein is about 0.70, 0.60, 0.50, 0.40, 0.30, 0.20, 0.15, 0.14, 0.13, 0.12, 0.10, 0.09, 0.08, 0.07, 0.06, 0.05, 0.04, 0.03, 0.02 times less than the difference in range of the 95% CI of the current classifier or clinical variable. The classifier or biomarker disclosed herein is more accurate than a current classifier or clinical variable when difference in range of the 95% CI of the classifier or biomarker disclosed herein between about 0.20 to about 0.04 times less than the difference in range of the 95% CI of the current classifier or clinical variable.

In some instances, the methods disclosed herein may comprise the use of a genomic classifier (GC) model. A general method for developing a GC model may comprise (a) providing a sample from a subject suffering from a cancer; (b) assaying the expression level for a plurality of targets; (c) generating a model by using a machine learning algorithm. In some instances, the machine learning algorithm comprises Random Forests. In another example, a GC model may developed by using a machine learning algorithm to analyze and rank genomic features. Analyzing the genomic features may comprise classifying one or more genomic features. The method may further comprise validating the classifier and/or refining the classifier by using a machine learning algorithm.

The methods disclosed herein may comprise generating one or more clinical classifiers (CC). The clinical classifier can be developed using one or more clinicopathologic variables. The clinicopathologic variables may be selected from the group comprising Lymph node invasion status (LNI); Surgical Margin Status (SMS); Seminal Vesicle Invasion (SVI); Extra Capsular Extension (ECE); Pathological Gleason Score; and the pre-operative PSA. The clinicopathologic variables may be selected from the group comprising Tumor Stage (e.g., CIS, Ta, T1, T2, T2a, T2b, T3, T3a, T4a, T4b), Nodal Status (e.g., NO, NI, N2, or N3), Lymphovascular invasion, age, or gender. The method may comprise using one or more of the clinicopathologic variables as binary variables. Alternatively, or additionally, the one or more clinicopathologic variables may be converted to a logarithmic value (e.g., log 10). The method may further comprise assembling the variables in a logistic regression. In some instances, the CC is combined with the GC to produce a genomic clinical classifier (GCC).

In some instances, the methods disclosed herein may comprise the use of a genomic-clinical classifier (GCC) model. A general method for developing a GCC model may comprise (a) providing a sample from a subject suffering from a cancer; (b) assaying the expression level for a plurality of targets; (c) generating a model by using a machine learning algorithm. In some instances, the machine learning algorithm comprises Random Forests.

### Cancer

The systems, compositions and methods disclosed herein may be used to diagnosis, monitor and/or predict the status or outcome of a cancer. Generally, a cancer is characterized by the uncontrolled growth of abnormal cells anywhere in a body. The abnormal cells may be termed cancer cells, malignant cells, or tumor cells. Many cancers and the abnormal cells that compose the cancer tissue are further identified by the name of the tissue that the abnormal cells originated from (for example, bladder cancer, lung cancer, colon cancer, prostate cancer, pancreatic cancer, thyroid cancer). Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a bladder cancer. Alternatively, the cancer may be a thyroid cancer, prostate cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavitity (pleura) or lining of the abdomen (peritoneum). The mesothelioma may be due to asbestos exposure. The cancer may be a brain cancer, such as a glioblastoma.

In some instances, the cancer is a bladder cancer. Bladder cancer is the fourth most common type of cancer in men and the ninth most common cancer in women. Invasive bladder cancer has a high propensity for recurrence. In some instances, the bladder cancer can be non-invasive bladder cancer, muscle-invasive bladder cancer, or advanced bladder cancer.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Stemberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1M0 or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

Alternatively, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage I often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than ⅓ of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

TNM classification may be used as the staging system for bladder tumors. In this aspect, the staging system takes into account how deep the tumor has grown into the bladder, whether there is cancer in the lymph nodes and whether the cancer has spread to any other part of the body. According to the TNM staging system, bladder cancer can be staged as follows: In bladder cancer stage 0, cancer cells are confined to the mucosa. In bladder cancer stage I the tumor invades the subepithelial connective tissue/lamina propria. In bladder cancer stage II cancer cells have invaded the muscularis propria but the tumor is still organ-confined. In bladder cancer stage III cancer cells have extended through the bladder wall to the perivesical tissue or to the Prostatic stroma, uterus or vagina. In bladder cancer stage IV cancer cells have proliferated to the lymph nodes, pelvic or abdominal wall, and/or other organs. The “bladder cancer” in the context of the present invention, may encompass any of the aforementioned stages.

### Markers for Bladder Cancer

Several biomarkers for bladder cancer diagnosis have been described. The BTA-Stat test detects complement factor H (CFH) and related proteins (CFH-rp). The nuclear matrix protein 22 (NMP22) test (Matritech, Newton, Mass.) is a double monoclonal antibody test designed to measure quantitatively the nuclear mitotic apparatus (MUMA) protein. The ImmunoCyt test (Diagno-Cure Inc., Sainte-Foy, Quebec Canada) detects bladder cancer markers present on exfoliated cells using a cocktail of fluorescent antibodies (19A211, M344 and LDQ10). The UroVysion test (Vysis Chicago, Ill.) employs centromere probes specific to chromosomes 3, 7, 17 and 9 to detect aneuploidy associated with bladder cancer. Other markers for bladder cancer include survivin, cytokeratins, telomerase, BLCA-4, microsatellite detection, hyaluronic acid and hyaluronidase, urine fibronectin, and chorionic gonadotropin. Use of these markers in combination with the markers and genomic classifiers of the present invention are specifically contemplated herein.

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

As used herein the term “tumor margin” refers to the tissue surrounding a discernible tumor. In the case of surgical removal of a solid tumor, the tumor margin is the tissue cut away with the discernible tumor that usually appears to be normal to the naked eye. More particularly, as used herein, “margin” refers to the edge, border or boundary of a tumor. The margin generally extends from about 1 mm to about 4 mm from the primary tumor but can be greater depending upon the size of the primary solid tumor. As used herein, the terms “surgical margin”, “tumor free margin”, “free margin”, “normal skin margin”, and “normal tissue margin” refer to the visible normal tissue or skin margin that is removed with the surgical excision of a tumor, growth, or malignancy. Surgical margin as read in a pathology report define the histological measurement of normal or unaffected tissue surrounding the visible tumor under a microscope on a glass mounted histology section. A “narrow” surgical margin implies that the tumor exists very close to the surgical margin, and a “wide” surgical margin implies the tumor exists far from the cut edge or the surgical margin.

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agens may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

Chemotherapeutic agents may be used alone or in combination. Examples of therapeutic combinations used to treat bladder cancer include: Gemcitabine and cisplatin; Methotrexate, vinblastine, doxorubicin (Adriamycin), and cisplatin (called M-VAC); Carboplatin and either paclitaxel or docetaxel (for patients with poor kidney function).

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therepy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a bladder tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permament brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (1311) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine 1131 tositumomab (Bexxar®) may be used for the treatment of certain types of cancer, such as NHL. In this regimen, nonradioactive tositumomab antibodies may be given to patients first, followed by treatment with tositumomab antibodies that have 1311 attached. Tositumomab may recognize and bind to the same protein on B lymphocytes as ibritumomab. The nonradioactive form of the antibody may help protect normal B lymphocytes from being damaged by radiation from 1311.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

In certain cases, radiation therapy is combined with a chemotherapeutic agent. Examples of chemotherapeutic agents which are used in combination with radiation to treat bladder cancer include Cisplatin; Cisplatin plus fluorouracil (5-FU); and Mitomycin with 5-FU.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and bladder cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, bladder, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as bladder, colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, bladder, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, bladder, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty, fifty or more of the target sequences corresponding to a target selected from Table 2B, Table 16 or SEQ ID NOs:1-1440, the subsets described herein, or a combination thereof. A patient report is also provided comprising a representation of measured expression levels of a plurality of target sequences in a biological sample from the patient, wherein the representation comprises expression levels of target sequences corresponding to 40, 50, 60, 70, 80, 90, 100 or more of the target sequences corresponding to a target selected from Table 2B, Table 16 or SEQ ID NOs:1-1440, the subsets described herein, or a combination thereof. or more coding targets and/or non-coding targets selected from Table 2B, Table 16 or SEQ ID NOs:1-1440. A patient report is also provided comprising a representation of measured expression levels of a plurality of target sequences in a biological sample from the patient, wherein the representation comprises expression levels of target sequences corresponding to 100, 125, 150, 175, 200, 225, 250, 275, 300 or more of the target sequences corresponding to a target selected from Table 2B, Table 16 or SEQ ID NOs:1-1440, the subsets described herein, or a combination thereof. A patient report is also provided comprising a representation of measured expression levels of a plurality of target sequences in a biological sample from the patient, wherein the representation comprises expression levels of target sequences corresponding to 300, 325, 350, 375, 400, 425, 450, 475, 500, 525, 550, 575, 600 or more of the target sequences corresponding to a target selected from Table 2B, Table 16 or SEQ ID NOs:1-1440, the subsets described herein, or a combination thereof. A patient report is also provided comprising a representation of measured expression levels of a plurality of target sequences in a biological sample from the patient, wherein the representation comprises expression levels of target sequences corresponding to 600, 625, 650, 675, 700, 725, 750, 775, 800, 825, 850 or more of the target sequences corresponding to a target selected from Table 2B, Table 16 or SEQ ID NOs:1-1440, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### EXEMPLARY EMBODIMENTS

Disclosed herein, in some embodiments, is a method for diagnosing, predicting, and/or monitoring a status or outcome of a cancer a subject, comprising: (a) assaying an expression level of a plurality of targets in a sample from the subject, wherein at least one target of the plurality of targets is selected from the group consisting of targets identified in Table 1; and (b) for diagnosing, predicting, and/or monitoring a status or outcome of a cancer based on the expression levels of the plurality of targets. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a thyroid cancer. In some embodiments, the cancer is a bladder cancer. In some embodiments, the cancer is a lung cancer. In some embodiments, the method further comprises assaying an expression level of a coding target. In some instances, the coding target is selected from the group consisting of targets identified in Table 2B, Table 16 or SEQ ID NOs:1-1440. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 2B, Table 16 or SEQ ID NOs:1-1440. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 2B, Table 16 or SEQ ID NOs:1-1440. In some instances, the plurality of targets comprises 5 or more targets selected from the group of targets identified in Table 2B, Table 16 or SEQ ID NOs:1-1440. In some instances, the plurality of targets comprises 10 or more targets selected from the group of targets identified in Table 2B, Table 16 or SEQ ID NOs:1-1440. In some instances, the plurality of targets comprises 15 or more targets selected from the group of targets identified in Table 2B, Table 16 or SEQ ID NOs:1-1440. In some instances, the plurality of targets comprises 20 or more targets selected from the group of targets identified in Table 2B, Table 16 or SEQ ID NOs:1-1440. In some instances, the plurality of targets comprises 25 or more targets selected from the group of targets identified in Table 2B, Table 16 or SEQ ID NOs:1-1440. In some instances, the plurality of targets comprises 30 or more targets selected from the group of targets identified in Table 2B, Table 16 or SEQ ID NOs:1-1440. In some instances, the plurality of targets comprises 35 or more targets selected from the group of targets identified in Table 2B, Table 16 or SEQ ID NOs:1-1440. In some instances, the plurality of targets comprises 40 or more targets selected from the group of targets identified in Table 2B, Table 16 or SEQ ID NOs:1-1440. In some embodiments, assaying the expression level comprises detecting and/or quantifying a nucleotide sequence of the plurality of targets. Alternatively, assaying the expression level comprises detecting and/or quantifying a polypeptide sequence of the plurality of targets. In some embodiments, assaying the expression level comprises detecting and/or quantifying the DNA levels of the plurality of targets. In some embodiments, assaying the expression level comprises detecting and/or quantifying the RNA or mRNA levels of the plurality of targets. In some embodiments, assaying the expression level comprises detecting and/or quantifying the protein level of the plurality of targets. In some embodiments, the diagnosing, predicting, and/or monitoring the status or outcome of a cancer comprises determining the malignancy of the cancer. In some embodiments, the diagnosing, predicting, and/or monitoring the status or outcome of a cancer includes determining the stage of the cancer. In some embodiments, the diagnosing, predicting, and/or monitoring the status or outcome of a cancer includes assessing the risk of cancer recurrence. In some embodiments, diagnosing, predicting, and/or monitoring the status or outcome of a cancer may comprise determining the efficacy of treatment. In some embodiments, diagnosing, predicting, and/or monitoring the status or outcome of a cancer may comprise determining a therapeutic regimen. Determining a therapeutic regimen may comprise administering an anti-cancer therapeutic. Alternatively, determining the treatment for the cancer may comprise modifying a therapeutic regimen. Modifying a therapeutic regimen may comprise increasing, decreasing, or terminating a therapeutic regimen.

Further disclosed, in some embodiments, is method for determining a treatment for a cancer in a subject, comprising: a) assaying an expression level of a plurality of targets in a sample from the subject, wherein at least one target of the plurality of targets is selected from the group consisting of targets identified in Table 1; and b) determining the treatment for a cancer based on the expression levels of the plurality of targets. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some embodiments, the coding target is selected from a sequence listed in Table 2B, Table 16 or SEQ ID NOs:1-1440. In some embodiments, the method further comprises assaying an expression level of a coding target. In some instances, the coding target is selected from the group consisting of targets identified in Table 2B, Table 16 or SEQ ID NOs:1-1440. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 2B, Table 16 or SEQ ID NOs:1-1440. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 2B, Table 16 or SEQ ID NOs:1-1440. In some instances, the plurality of targets comprises 5 or more targets selected from the group of targets identified in Table 2B, Table 16 or SEQ ID NOs:1-1440. In some instances, the plurality of targets comprises 10 or more targets selected from the group of targets identified in Table 2B, Table 16 or SEQ ID NOs:1-1440. In some instances, the plurality of targets comprises 15 or more targets selected from the group of targets identified in Table 2B, Table 16 or SEQ ID NOs:1-1440. In some instances, the plurality of targets comprises 20 or more targets selected from the group of targets identified in Table 2B, Table 16 or SEQ ID NOs:1-1440. In some instances, the plurality of targets comprises 25 or more targets selected from the group of targets identified in Table 2B, Table 16 or SEQ ID NOs:1-1440. In some instances, the plurality of targets comprises 30 or more targets selected from the group of targets identified in Table 2B, Table 16 or SEQ ID NOs:1-1440. In some instances, the plurality of targets comprises 35 or more targets selected from the group of targets identified in Table 2B, Table 16 or SEQ ID NOs:1-1440. In some instances, the plurality of targets comprises 40 or more targets selected from the group of targets identified in Table 2B, Table 16 or SEQ ID NOs:1-1440. In some embodiments, assaying the expression level comprises detecting and/or quantifying a nucleotide sequence of the plurality of targets. In some embodiments, determining the treatment for the cancer includes determining the efficacy of treatment. Determining the treatment for the cancer may comprise administering an anti-cancer therapeutic. Alternatively, determining the treatment for the cancer may comprise modifying a therapeutic regimen. Modifying a therapeutic regimen may comprise increasing, decreasing, or terminating a therapeutic regimen.

The methods use the probe sets, probes and primers described herein to provide expression signatures or profiles from a test sample derived from a subject having or suspected of having cancer. In some embodiments, such methods involve contacting a test sample with a probe set comprising a plurality of probes under conditions that permit hybridization of the probe(s) to any target nucleic acid(s) present in the test sample and then detecting any probe:target duplexes formed as an indication of the presence of the target nucleic acid in the sample. Expression patterns thus determined are then compared to one or more reference profiles or signatures. Optionally, the expression pattern can be normalized. The methods use the probe sets, probes and primers described herein to provide expression signatures or profiles from a test sample derived from a subject to classify the cancer as recurrent or non-recurrent.

In some embodiments, such methods involve the specific amplification of target sequences nucleic acid(s) present in the test sample using methods known in the art to generate an expression profile or signature which is then compared to a reference profile or signature.

In some embodiments, the invention further provides for prognosing patient outcome, predicting likelihood of recurrence after cystectomy and/or for designating treatment modalities.

In one embodiment, the methods generate expression profiles or signatures detailing the expression of the target sequences having altered relative expression with different cancer outcomes. In some embodiments, the methods detect combinations of expression levels of sequences exhibiting positive and negative correlation with a disease status. In one embodiment, the methods detect a minimal expression signature.

The gene expression profiles of each of the target sequences comprising the portfolio can fixed in a medium such as a computer readable medium. This can take a number of forms. For example, a table can be established into which the range of signals (e.g., intensity measurements) indicative of disease or outcome is input. Actual patient data can then be compared to the values in the table to determine the patient samples diagnosis or prognosis. In a more sophisticated embodiment, patterns of the expression signals (e.g., fluorescent intensity) are recorded digitally or graphically.

The expression profiles of the samples can be compared to a control portfolio. The expression profiles can be used to diagnose, predict, or monitor a status or outcome of a cancer. For example, diagnosing, predicting, or monitoring a status or outcome of a cancer may comprise diagnosing or detecting a cancer, cancer metastasis, or stage of a cancer. In other instances, diagnosing, predicting, or monitoring a status or outcome of a cancer may comprise predicting the risk of cancer recurrence. Alternatively, diagnosing, predicting, or monitoring a status or outcome of a cancer may comprise predicting mortality or morbidity.

Further disclosed herein are methods for characterizing a patient population. Generally, the method comprises: (a) providing a sample from a subject; (b) assaying the expression level for a plurality of targets in the sample; and (c) characterizing the subject based on the expression level of the plurality of targets. In some embodiments, the method further comprises assaying an expression level of a coding target. In some instances, the coding target is selected from the group consisting of targets identified in Table 2B, Table 16 or SEQ ID NOs:1-1440. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 2B, Table 16 or SEQ ID NOs:1-1440. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 2B, Table 16 or SEQ ID NOs:1-1440. In some instances, the plurality of targets comprises 5 or more targets selected from the group of targets identified in Table 2B, Table 16 or SEQ ID NOs:1-1440. In some instances, the plurality of targets comprises 10 or more targets selected from the group of targets identified in Table 2B, Table 16 or SEQ ID NOs:1-1440. In some instances, the plurality of targets comprises 15 or more targets selected from the group of targets identified in Table 2B, Table 16 or SEQ ID NOs:1-1440. In some instances, the plurality of targets comprises 20 or more targets selected from the group of targets identified in Table 2B, Table 16 or SEQ ID NOs:1-1440. In some instances, the plurality of targets comprises 25 or more targets selected from the group of targets identified in Table 2B, Table 16 or SEQ ID NOs:1-1440. In some instances, the plurality of targets comprises 30 or more targets selected from the group of targets identified in Table 2B, Table 16 or SEQ ID NOs:1-1440. In some instances, the plurality of targets comprises 35 or more targets selected from the group of targets identified in Table 2B, Table 16 or SEQ ID NOs:1-1440. In some instances, the plurality of targets comprises 40 or more targets selected from the group of targets identified in Table 2B, Table 16 or SEQ ID NOs:1-1440. In some embodiments, assaying the expression level comprises detecting and/or quantifying a nucleotide sequence of the plurality of targets. In some instances, the method may further comprise diagnosing a cancer in the subject. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

Further disclosed herein are methods for selecting a subject suffering from a cancer for enrollment into a clinical trial. Generally, the method comprises: (a) providing a sample from a subject; (b) assaying the expression level for a plurality of targets in the sample; and (c) characterizing the subject based on the expression level of the plurality of targets. In some embodiments, the method further comprises assaying an expression level of a coding target. In some instances, the coding target is selected from the group consisting of targets identified in Table 2B, Table 16 or SEQ ID NOs:1-1440. In some embodiments, the coding target is an exon-coding transcript. In some embodiments, the exon-coding transcript is an exonic sequence. In some embodiments, the method further comprises assaying an expression level of a non-coding target. In some instances, the non-coding target is selected from the group consisting of targets identified in Table 2B, Table 16 or SEQ ID NOs:1-1440. In some instances, the non-coding target is a non-coding transcript. In other instances, the non-coding target is an intronic sequence. In other instances, the non-coding target is an intergenic sequence. In some instances, the non-coding target is a UTR sequence. In other instances, the non-coding target is a non-coding RNA transcript. In some embodiments, the target comprises a nucleic acid sequence. In some embodiments, the nucleic acid sequence is a DNA sequence. In some embodiments, the nucleic acid sequence is an RNA sequence. In other instances, the target comprises a polypeptide sequence. In some instances, the plurality of targets comprises 2 or more targets selected from the group of targets identified in Table 2B, Table 16 or SEQ ID NOs:1-1440. In some instances, the plurality of targets comprises 5 or more targets selected from the group of targets identified in Table 2B, Table 16 or SEQ ID NOs:1-1440. In some instances, the plurality of targets comprises 10 or more targets selected from the group of targets identified in Table 2B, Table 16 or SEQ ID NOs:1-1440. In some instances, the plurality of targets comprises 15 or more targets selected from the group of targets identified in Table 2B, Table 16 or SEQ ID NOs:1-1440. In some instances, the plurality of targets comprises 20 or more targets selected from the group of targets identified in Table 2B, Table 16 or SEQ ID NOs:1-1440. In some instances, the plurality of targets comprises 25 or more targets selected from the group of targets identified in Table 2B, Table 16 or SEQ ID NOs:1-1440. In some instances, the plurality of targets comprises 30 or more targets selected from the group of targets identified in Table 2B, Table 16 or SEQ ID NOs:1-1440. In some instances, the plurality of targets comprises 35 or more targets selected from the group of targets identified in Table 2B, Table 16 or SEQ ID NOs:1-1440. In some instances, the plurality of targets comprises 40 or more targets selected from the group of targets identified in Table 2B, Table 16 or SEQ ID NOs:1-1440. In some embodiments, assaying the expression level comprises detecting and/or quantifying a nucleotide sequence of the plurality of targets. In some instances, the method may further comprise diagnosing a cancer in the subject. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the bladder cancer is non-invasive bladder cancer, muscle-invasive bladder cancer, or advanced bladder cancer. In some embodiments, the cancer is a prostate cancer. In some embodiments, the cancer is a pancreatic cancer. In some embodiments, the cancer is a bladder cancer. In some embodiments, the cancer is a thyroid cancer. In some embodiments, the cancer is a lung cancer. In some instances, characterizing the subject comprises determining whether the subject would respond to an anti-cancer therapy. Alternatively, characterizing the subject comprises identifying the subject as a non-responder to an anti-cancer therapy. Optionally, characterizing the subject comprises identifying the subject as a responder to an anti-cancer therapy.

(Further disclosed herein is a method of analyzing a cancer in an individual in need thereof, comprising (a) obtaining an expression profile from a sample obtained from the individual, wherein the expression profile comprises one or more targets selected from Table 2B, Table 16 or SEQ ID NOs:1-1440; and (b) comparing the expression profile from the sample to an expression profile of a control or standard. In some embodiments, the plurality of targets comprises at least 5 targets selected from Table 2B, Table 16 or SEQ ID NOs:1-1440. In some embodiments, wherein the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the method further comprises providing diagnostic or prognostic information to the individual about the cardiovascular disorder based on the comparison. In some embodiments, the method further comprises diagnosing the individual with a cancer if the expression profile of the sample (a) deviates from the control or standard from a healthy individual or population of healthy individuals, or (b) matches the control or standard from an individual or population of individuals who have or have had the cancer. In some embodiments, the method further comprises predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the method further comprises predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer. In some embodiments, the deviation is the expression level of one or more targets from the sample is greater than the expression level of one or more targets from a control or standard derived from a healthy individual or population of healthy individuals. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs: 1-1440 or a combination thereof. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

Also disclosed herein is a method of diagnosing cancer in an individual in need thereof, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) diagnosing a cancer in the individual if the expression profile of the sample (i) deviates from the control or standard from a healthy individual or population of healthy individuals, or (ii) matches the control or standard from an individual or population of individuals who have or have had the cancer. In some embodiments, the plurality of targets comprises at least 5 targets selected from Table 2B, Table 16 or SEQ ID NOs:1-1440. In some embodiments, wherein the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

In some embodiments is a method of predicting whether an individual is susceptible to developing a cancer, comprising (a) obtaining an expression profile from a sample obtained from the individual, wherein the expression profile comprises one or more targets selected from Table 1; (b) comparing the expression profile from the sample to an expression profile of a control or standard; and (c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, wherein the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

In some embodiments is a method of predicting an individual's response to a treatment regimen for a cancer, comprising: (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) predicting the individual's response to a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, wherein the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

A method of prescribing a treatment regimen for a cancer to an individual in need thereof, comprising (a) obtaining an expression profile from a sample obtained from the individual, wherein the expression profile comprises one or more targets selected from Table 2B, Table 16 or SEQ ID NOs:1-1440; (b) comparing the expression profile from the sample to an expression profile of a control or standard; and (c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas. In some embodiments, the method further comprises a software module executed by a computer-processing device to compare the expression profiles. In some embodiments, the deviation is the expression level of one or more targets from the sample is at least about 30% greater than the expression level of one or more targets from a control or standard derived from a healthy individual or population of healthy individuals. In some embodiments, the deviation is the expression level of one or more targets from the sample is less than the expression level of one or more targets from a control or standard derived from a healthy individual or population of healthy individuals. In some embodiments, the deviation is the expression level of one or more targets from the sample is at least about 30% less than the expression level of one or more targets from a control or standard derived from a healthy individual or population of healthy individuals. In some embodiments, the method further comprises using a machine to isolate the target or the probe from the sample. In some embodiments, the method further comprises contacting the sample with a label that specifically binds to the target, the probe, or a combination thereof. In some embodiments, the method further comprises contacting the sample with a label that specifically binds to a target selected from Table 2B, Table 16 or SEQ ID NOs:1-1440. In some embodiments, the method further comprises amplifying the target, the probe, or any combination thereof. In some embodiments, the method further comprises sequencing the target, the probe, or any combination thereof. In some embodiments, the method further comprises converting the expression levels of the target sequences into a likelihood score that indicates the probability that a biological sample is from a patient who will exhibit no evidence of disease, who will exhibit systemic cancer, or who will exhibit biochemical recurrence. In some embodiments, the target sequences are differentially expressed the cancer. In some embodiments, the differential expression is dependent on aggressiveness. In some embodiments, the expression profile is determined by a method selected from the group consisting of RT-PCR, Northern blotting, ligase chain reaction, array hybridization, and a combination thereof.

Further disclosed herein is a kit for analyzing a cancer, comprising (a) a probe set comprising a plurality of target sequences, wherein the plurality of target sequences comprises at least one target sequence listed in Table 2B, Table 16 or SEQ ID NOs:1-1440; and (b) a computer model or algorithm for analyzing an expression level and/or expression profile of the target sequences in a sample. In some embodiments, the kit further comprises a computer model or algorithm for correlating the expression level or expression profile with disease state or outcome. In some embodiments, the kit further comprises a computer model or algorithm for designating a treatment modality for the individual. In some embodiments, the kit further comprises a computer model or algorithm for normalizing expression level or expression profile of the target sequences. In some embodiments, the kit further comprises a computer model or algorithm comprising a robust multichip average (RMA), frozen robust multichip average (fRMA), Single Channel Array Normalization (SCAN), ComBat (Combining Batches of gene expression), probe logarithmic intensity error estimation (PLIER), non-linear fit (NLFIT) quantile-based, nonlinear normalization, or a combination thereof. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

Further disclosed herein is a system for analyzing a cancer, comprising (a) one or more probe sets comprising a plurality of target sequences, wherein (i) the plurality of target sequences hybridizes to one or more targets selected from Table 2B, Table 16 or SEQ ID NOs:1-1440; or (ii) the plurality of target sequences comprises one or more target sequences selected from Table 2B, Table 16 or SEQ ID NOs:1-1440; and (b) a computer model or algorithm for analyzing an expression level and/or expression profile of the target hybridized to the probe in a sample from a subject suffering from a cancer. In some embodiments, the system further comprises electronic memory for capturing and storing an expression profile. In some embodiments, the system further comprises a computer-processing device, optionally connected to a computer network. In some embodiments, the system further comprises a software module executed by the computer-processing device to analyze an expression profile. In some embodiments, the system further comprises a software module executed by the computer-processing device to compare the expression profile to a standard or control. In some embodiments, the system further comprises a software module executed by the computer-processing device to determine the expression level of the target. In some embodiments, the system further comprises a machine to isolate the target or the probe from the sample. In some embodiments, the system further comprises a machine to sequence the target or the probe. In some embodiments, the system further comprises a machine to amplify the target or the probe. In some embodiments, the system further comprises a label that specifically binds to the target, the probe, or a combination thereof. In some embodiments, the system further comprises a software module executed by the computer-processing device to transmit an analysis of the expression profile to the individual or a medical professional treating the individual. In some embodiments, the system further comprises a software module executed by the computer-processing device to transmit a diagnosis or prognosis to the individual or a medical professional treating the individual. In some embodiments, the plurality of targets comprises at least 5 targets selected from Table 2B, Table 16 or SEQ ID NOs:1-1440. In some embodiments, the plurality of targets comprises at least 10 targets selected from Table 2B, Table 16 or SEQ ID NOs:1-1440. In some embodiments, the plurality of targets comprises at least 15 targets selected from Table 2B, Table 16 or SEQ ID NOs:1-1440. In some embodiments, the plurality of targets comprises at least 20 targets selected from Table 2B, Table 16 or SEQ ID NOs:1-1440. In some embodiments, the cancer is selected from the group consisting of a carcinoma, sarcoma, leukemia, lymphoma, myeloma, and a CNS tumor. In some embodiments, the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

### EXAMPLES

### Example 1

### A 15 Marker Genomic Classifier Predicts Risk for Recurrence After Cystectomy in

Muscle Invasive Bladder Cancer Patients.

A 15-marker genomic classifier containing biologically relevant RNA sequences using FFPE tumor tissue specimens obtained from a large cohort of patients who underwent radical cystectomy for muscle invasive bladder cancer was developed as follows. Bladder cancer patients who underwent radical cystectomy with curative intent formed the primary source of study material. From this population, a representative set of 225 patients with organ-confined, muscle-invasive (pT2N0M0), extravesical (pT3-4aN0M0), and node-positive (pTanyN1-3M0) UCB was identified. Each patient had a minimum post-cystectomy follow-up of two years unless they died of UCB prior to that date. Two-thirds of the cohort of 225 patients were assigned to a discovery set and the balance one-third to a validation set. The clinical end-point used to select discover biomarkers was recurrence-free survival (RFS) duration.

### Specimen Processing and Initial Microarray Analysis.

Archival formalin-fixed paraffin-embedded (FFPE) primary tumor specimens of the 225 study patients were obtained for histopathological re-review. Hematoxylin/eosin-stained sections were used to guide tumor specimen selection using a 1 mm diameter core tissue microarray punch tool (Beecher Instruments, Sun Prairie, Wis.). Total RNA was extracted and purified using RNeasy FFPE kit (Qiagen, Valencia, Calif.). RNA was amplified and labeled using the Ovation WTA FFPE system (NuGen, San Carlos, Calif.) and hybridized to Human Exon 1.0 ST microarrays (Affymetrix, Santa Clara, Calif.) according to the manufacturer's recommendations. Human Exon GeneChips profile coding and non-coding regions of the entire human transcriptome using probe selection regions (PSRs), hereinafter referred to as features.

### Patient Cohorts, Classifier Development and Application

Samples from 199 (88.4%) patient specimens passed microarray quality control. To identify features most clinically relevant to RFS, area under the receiver-operating characteristic (ROC) curve, t-test statistics and median fold difference (MFD) were calculated in the discovery cohort. Based on these metrics, features were filtered by area under ROC curve (AUC) >0.6, unadjusted t-test P<0.050 and MFD>1.5. Selected features were combined to produce a genomic classifier (GC) score using a random forest algorithm with 50,000 trees based on the discovery set. Each tree generated by the random forest algorithm was based on a bootstrapped subset of the discovery set. In each bootstrapping iteration, an “out-of-bag” subset of patients was excluded from the creation of the decision tree. The out-of-bag error rate was estimated based on the misclassification rate of this group. The selection of random forest mtry and nodesize parameters was based on minimizing the out-of-bag error rate. The GC outputs a continuous score between 0 and 1, with higher scores indicating higher probability of recurrence.

The prognostic ability of GC was benchmarked against two clinical nomograms: the International Bladder Cancer Nomogram Consortium (IBCNC) (Bochner et al. 2006) and an in-house “clinical-only” classifier (CC). The latter was developed on the discovery set and incorporated age, gender, pathological stage, and lymphovascular invasion status modeled using logistic regression. Additionally, to evaluate the joint prognostic value of genomic information and clinicopathologic variables, GC was combined with IBCNC and CC by logistic regression in the discovery set into integrated G-IBCNC and G-CC, respectively. Analogous to GC, patients were scored using the above classifiers between 0 and 1. For CC and G-CC, interactions between different clinicopathologic factors were explored in the discovery cohort and applied to the model when significant. The locked genomic, clinical and genomic-clinical models were then applied to patients in the validation cohort in a blinded fashion.

Statistical Analyses

Univariable prognostic abilities of classifiers were compared using discrimination boxplots, Wilcoxon rank-sum test and logistic regression. Cumulative incidence curves for RFS were constructed using Fine-Gray competing risks analysis. (Fine and Gray 1999) Deaths were considered a competing risk. Concordance summary index, an extension of AUC for censored data, was also used to compare performance of genomic classifiers, external signatures and clinical nomograms with respect to RFS. For the analyses where GC was categorized, majority rule criterion with classifier scores of >0.5 and ≦0.5 grouped as high-risk and low-risk, respectively was used. Analyses were performed using R v2.15.2. All tests were two-sided with type I error probability of 5%.

Time-dependent survival ROC curves were evaluated for prediction of recurrence within four years post-cystectomy. (Heagerty et al. 2000) For survival ROC, the nearest-neighbor estimator with λ=0.002 was used to approximate survival function density. The 95% confidence intervals (Cis) for survival-ROC AUCs were approximated through bootstrapping. Decision curve analyses were used to assess the net clinical benefit of genomic versus clinical models across different threshold probabilities. (Vickers and Elkin 2006). Reassignment of patients to risk strata based on addition of genomic information was assessed using reclassification plots. (Pepe 2011).

Importance of genomic-based classifiers relative to individual and combined clinical information, and their independent prognostic abilities were evaluated by multivariable Cox regression models. Proportional hazards assumptions of the Cox model were confirmed by evaluating the scaled Schoenfeld residuals. (Grambsch and Therneau 1994). For univariable and multivariable analyses, classifier scores were assessed as continuous variables (step size=0.1) unless dichotomization was required. For the analyses where GC was categorized, majority rule criterion with classifier scores of >0.5 and ≦0.5 grouped as high-risk and low-risk, respectively was used. Estimates of censoring distribution were used to calculate follow-up duration. (Korn 1986).

### Analysis of Biological Interactions Between Prognostic Markers

To understand the biological interactions of constituent markers within GC, first-degree partners of their respective genes were extracted using the Human Signaling Network v5, a curated database with nearly 63,000 directed and undirected interactions between approximately 6,300 proteins. (Cui et al. 2007) Database for Annotation, Visualization and Integrated Discovery (DAVID) v6.7 was used to assess biological processes, molecular functions and Kyoto Encyclopedia of Genes and Genomes (KEGG) pathways of the gene network. (Kanehisa et al. 2004; Huang et al. 2009b; Huang et al. 2009a) Enriched processes, molecular functions or KEGG pathways with Benjamini-Hochberg-corrected P<0.05 were selected. (Benjamini and Hochberg 1995) Enrichment Map, a network-based gene set enrichment visualization method implemented as a Cytoscape plugin, was used to visualize the highly enriched biological concepts and group similar terms together. (Merico et al. 2010) Nodes in the Enrichment Map represent individual Gene Ontology (GO) terms or KEGG pathways and lines represent the amount of overlap between the terms.

### Discovery and Initial Performance of Genomic-Based Classifiers

To test the hypothesis that risk prediction models incorporating genomic expression signatures which may better characterize the biology that determines the propensity of tumor recurrence post-cystectomy than clinical variables alone, a genomic classifier (GC) was developed and tested on the discovery set. Clinical characteristics of the cohort of patients analyzed are listed in Table 1. Median patient age was 68.5 (IQR: 63.6-75.6) years. Median follow-up was 9.2 years; 68 (51.1%) patients recurred and 77 (57.9%) patients were dead at last follow-up.

Expressions of nearly 1.4 million RNA features in tumors of patients who recurred were compared to those who remained recurrence-free at last follow-up. Following normalization and feature selection, 15 markers were identified corresponding to RNAs from protein-coding and non-coding regions of the genome that were differentially expressed based on recurrence (Table 2A, Table 2B). A random forest machine-learning algorithm assembled these markers into a GC that assigned a score to each patient. GC performance was compared to that of individual clinicopathologic variables, the IBCNC postoperative nomogram, (Bochner et al. 2006) and a clinical-only classifier (CC) that represented an optimized clinicopathologic prognostic model developed on the discovery set.

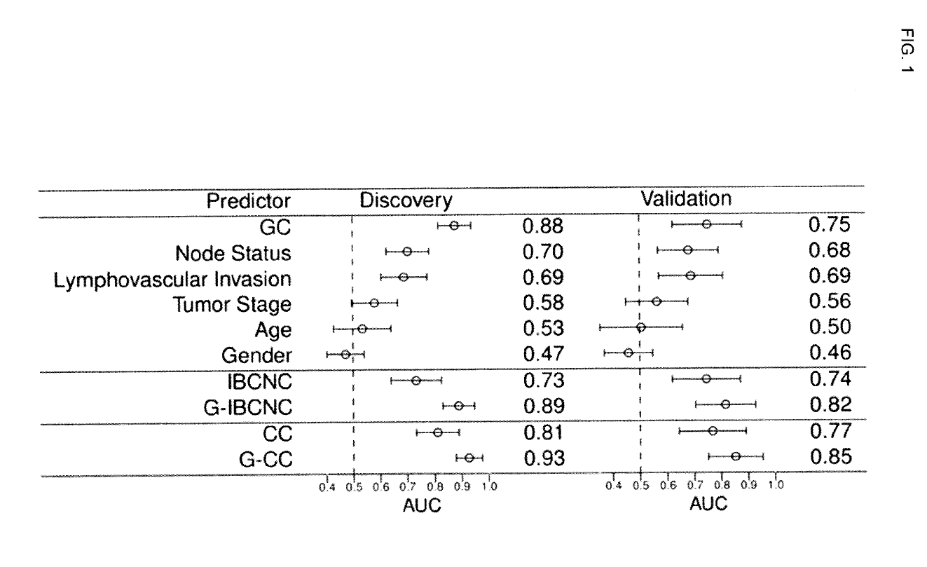

GC had an AUC of 0.88 (95% CI: 0.81-0.93) in the discovery set, with ENAH being the most prognostic individual marker within the signature (AUC=0.66). The performance of GC was higher than any individual clinicopathologic variable such as tumor and nodal stages, and lymphovascular invasion status (FIG. 1). The IBCNC and CC clinical risk prediction models had AUCs of 0.73 and 0.81, respectively. To examine whether the combination of clinical and genomic risk prediction models could boost model performance for prediction of recurrence GC was combined with IBCNC (G-IBCNC) and CC (G-CC). AUCs of these combined models in the discovery cohort were 0.89 (95% CI: 0.84-0.95) and 0.93 (95% CI: 0.88-0.97), respectively.

### Independent Validation of Genomic-Based Classifiers

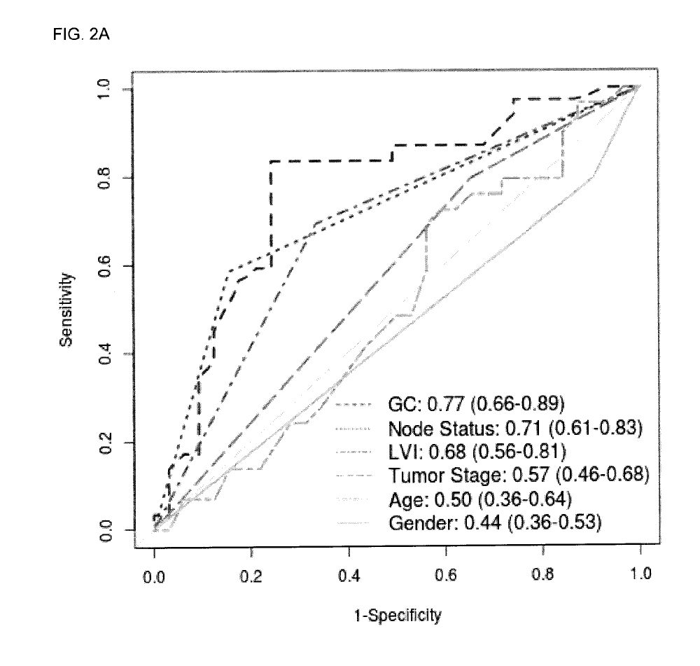

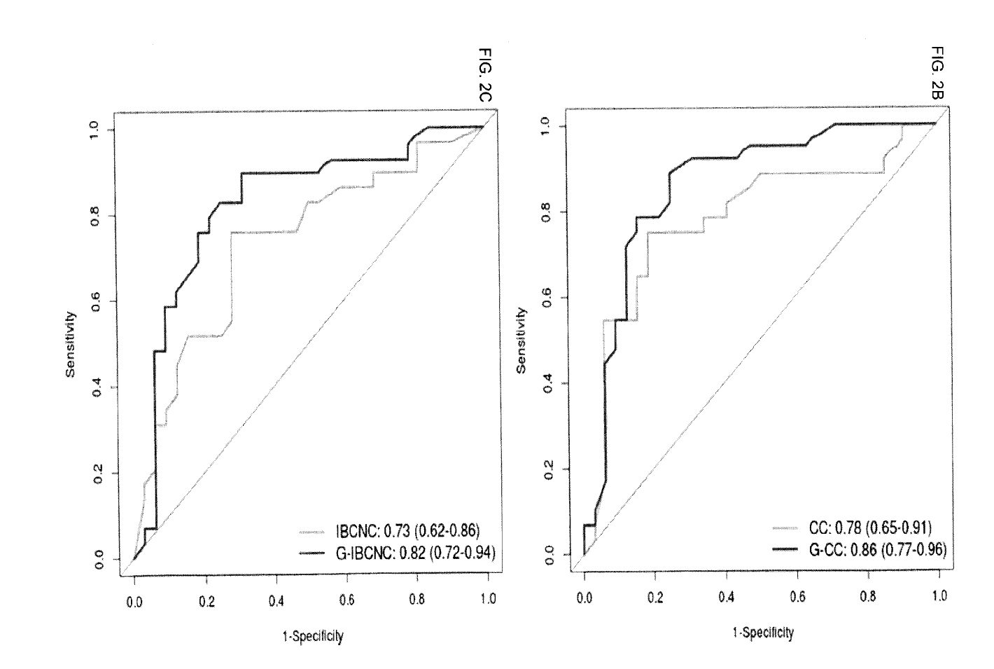

The prognostic performance of the locked classifiers was then assessed (with bioinformaticians blinded to clinical variables) on an independent validation set of 66 UCB patients. Clinical characteristics of patients in the validation and discovery sets were comparable (Table 1). Median patient age was 69.8 (IQR: 63.1-74.3) years. Median follow-up was 10.8 years; 33 (50%) patients recurred and 45 (68.2%) patients were dead by end of follow-up. By univariate analysis, GC (P=0.003), nodal status (P=0.006) and LVI (P=0.005) were prognostic for RFS, with GC having the highest hazard ratio (1.23 per every 10% increase in GC score; Table 3). GC performance as measured by standard ROC analysis was superior to single clinicopathologic variables (FIG. 1). This was confirmed by survival ROC analysis where AUC for GC (0.77; 95% CI: 0.65-0.90) was higher than any individual clinicopathologic variable (FIG. 2A).

TABLE 1

| | Discovery Cohort | | | | | | | Validation Cohort | | | | | | | |
|---|---|---|---|---|---|---|---|---|---|---|---|---|---|---|---|
| | | | | | | | Not | | | | | | | Not | |
| | | | Not | | | | recurred | | | Not | | | | recurred | Discovery vs. |
| | Total n | | recurred n | | Recurred n | | vs. Recurred | Total n | | recurred n | | Recurred n | | vs. Recurred | Validation |
| | (cohort %) | | (column %)* | | (column %)* | | P | (cohort %) | | (column %)* | | (column %)* | | P | P |
| Cohort | 133 | (100) | 65 | (48.9) | 68 | (51.1) | | 66 | (100) | 33 | (50.0) | 33 | (50.0) | | |
| (low %) | | | | | | | | | | | | | | | |
| Age | | | | | | | 0.49 | | | | | | | 1.00 | 0.55 |
| <70 years | 73 | (54.9) | 38 | (58.5) | 35 | (51.5) | | 33 | (50.0) | 17 | (51.5) | 16 | (48.5) | | |
| ≧70 years | 60 | (45.1) | 27 | (41.5) | 33 | (48.5) | | 33 | (50.0) | 18 | (48.5) | 17 | (51.5) | | |
| Gender | | | | | | | 0.82 | | | | | | | 0.73 | 1.00 |
| Male | 111 | (83.5) | 55 | (84.6) | 50 | (82.4) | | 56 | (84.8) | 29 | (87.9) | 27 | (81.8) | | |
| Female | 22 | (16.5) | 10 | (15.4) | 12 | (17.6) | | 10 | (15.2) | 4 | (12.1) | 6 | (18.2) | | |
| Race | | | | | | | 0.32 | | | | | | | 0.43 | 0.65 |
| Caucasian | 115 | (86.5) | 54 | (83.1) | 61 | (89.7) | | 59 | (89.4) | 31 | (93.9) | 28 | (84.8) | | |
| Not | 18 | (13.5) | 11 | (16.9) | 7 | (10.3) | | 7 | (10.6) | 2 | (6.1) | 5 | (15.2) | | |
| Caucasian | | | | | | | | | | | | | | | |
| Tumor Stage | | | | | | | 0.033 | | | | | | | 0.013 | 0.76 |
| pT1 | 3 | (2.3) | 1 | (1.5) | 2 | (0.0) | | 3 | (4.5) | 2 | (6.1) | 1 | (3.0) | | |
| pT2 | 44 | (33.1) | 28 | (43.1) | 16 | (23.5) | | 20 | (30.3) | 13 | (39.4) | 7 | (21.2) | | |
| pT3 | 58 | (43.6) | 28 | (43.1) | 30 | (44.1) | | 31 | (47.0) | 17 | (51.5) | 14 | (42.4) | | |
| pT4a | 28 | (21.0) | 8 | (12.3) | 20 | (29.4) | | 12 | (18.2) | 1 | (3.0) | 11 | (33.3) | | |
| Nodal Status | | | | | | | <0.001 | | | | | | | 0.011 | 0.64 |
| N0 | 88 | (66.2) | 56 | (86.2) | 32 | (47.1) | | 41 | (62.1) | 26 | (78.8) | 15 | (45.5) | | |
| N1 3 | 45 | (33.8) | 9 | (13.8) | 36 | (52.9) | | 25 | (37.9) | 7 | (21.2) | 18 | (54.5) | | |
| Lympho- | | | | | | | <0.001 | | | | | | | 0.005 | 1.00 |
| vascular | | | | | | | | | | | | | | | |
| Invasion | | | | | | | | | | | | | | | |
| Absent | 58 | (43.6) | 39 | (69.6) | 19 | (31.7) | | 30 | (45.5) | 20 | (69.0) | 10 | (31.3) | | |
| Present | 58 | (43.6) | 17 | (30.4) | 41 | (68.3) | | 51 | (47.0) | 9 | (51.0) | 22 | (68.7) | | |
| Adjuvant | | | | | | | 0.022 | | | | | | | 0.22 | 0.17 |
| Chemotherapy | | | | | | | | | | | | | | | |
| Not | 78 | (58.8) | 45 | (69.2) | 33 | (49.3) | | 32 | (48.5) | 19 | (57.6) | 13 | (30.4) | | |
| administered | | | | | | | | | | | | | | | |
| Administered | 54 | (40.6) | 20 | (30.8) | 34 | (50.7) | | 34 | (51.9) | 14 | (42.4) | 20 | (80.5) | | |
| Status at | | | | | | | <0.001 | | | | | | | <0.001 | 0.17 |
| Last Follow-up | | | | | | | | | | | | | | | |
| Alive | 56 | (42.1) | 50 | (76.9) | 6 | (8.8) | | 21 | (31.8) | 21 | (63.6) | 0 | (0.0) | | |
| Dead | 77 | (57.9) | 15 | (23.1) | 62 | (91.2) | | 45 | (68.2) | 12 | (36.4) | 33 | (100) | | |

P values based on Fisher's exact test
*Except where noted

### Clinicopathologic Characteristics of Patients in the Discovery and Validation Cohorts.

TABLE 2A

| | | | | | | Important Biological Process(es) | | | | |
|---|---|---|---|---|---|---|---|---|---|---|
| | | | Recurrence-specific | | | Cell | | | | |
| | | | Feature Metrics in | | | proliferation/ | | | Signaling | |
| | | | Discovery Cohort | | | Cell cycle | | | pathways/ | |
| Feature | Associated | Chromosomal | | t-test | | regulation/ | Cell differ- | Regulation of | Signal | |
| and Type | Gene | Location | AUC | P | MFD | Apoptosis | entiation | transcription | transduction | Other |
| 1^{a} | FOXO6 | 1p34.2 | 0.6251 | 0.0065 | 1.6514 | ✓ | | ✓ | ✓ | Angiogenesis |
| | | | | | | | | | | regulation |
| 2^{a} | HSD17B7 | 1q23 | 0.6234 | 0.0138 | 1.5401 | | | | | Cholesterol |
| | | | | | | | | | | metabolism |
| 3^{a} | ARID4B | 1q42.1-q43 | 0.6223 | 0.0310 | 1.5427 | | | ✓ | | Histone |
| | | | | | | | | | | methylation |
| 4^{a} | ENAH | 1q42.12 | 0.6617 | 0.0026 | 1.6644 | ✓ | | | ✓ | |
| 5^{b} | MAP4K3 | 2p22.1 | 0.6067 | 0.0327 | 1.5895 | ✓ | ✓ | | ✓ | |
| 6^{a} | MARCH7 | 2q24.2 | 0.6020 | 0.0275 | 1.5425 | | | | | Protein |
| | | | | | | | | | | ubiquitination |
| 7^{c} | MECOM | 3q26.2 | 0.6434 | 0.0045 | 1.5113 | ✓ | ✓ | ✓ | ✓ | |
| 8^{a} | LRBA | 4q31.3 | 0.6261 | 0.0081 | 1.5432 | ✓ | ✓ | | ✓ | |
| 9^{a} | MUT | 6p12.3 | 0.6206 | 0.0232 | 1.5150 | | | | | lipid metabolism |
| 10^{a} | CRCP | 7q11.21 | 0.6135 | 0.0259 | 1.5144 | ✓ | ✓ | ✓ | ✓ | |
| 11^{b} | SYPL1 | 7q22.3 | 0.6064 | 0.0485 | 1.5328 | | | | | |
| 12^{a} | ARFGEF1 | 8q13 | 0.6322 | 0.0354 | 1.5580 | | | | ✓ | |
| 13^{a} | EHF | 11p12 | 0.6437 | 0.0022 | 1.7470 | ✓ | | ✓ | | |
| 14^{a} | METTL7A | 12q13.12 | 0.6128 | 0.0201 | 1.6586 | | | | | Methyltransferase |
| | | | | | | | | | | activity |
| 15^{a} | PPP1R12A | 12q15.a21 | 0.6104 | 0.0406 | 1.5429 | ✓ | | ✓ | ✓ | Cell adhesion |

^{a}Exonic feature
^{b}Exonic antisense feature
^{c}Intronic antisense feature
Biological processes summarized using Gene Ontology Annotation database (Dimmer E C, Huntley R P, Alam-Fareque V, et al: The UniProt-GO Annotation database in 2011. Nucleic Acids Res 40: D565-D570. 2012).
Abbreviations: AUC, area under receiver operating characteristic curve; MFD, median told difference.

Summary description of markers comprising the genomic classifier for predicting post-cystectomy bladder cancer recurrence.

TABLE 2B

| SEQ ID NO | Affymetrix Probeset ID | Gene | Category | Chromosome | Start | End | Strand |
|---|---|---|---|---|---|---|---|
| SEQ ID NO. 1 | 3326487 | EHF | Coding | chr11 | 34673110 | 34673157 | + |
| SEQ ID NO. 2 | 2458376 | ENAH | Coding | chr1 | 225692693 | 225692726 | − |
| SEQ ID NO. 3 | 3414753 | METTL7A | Non coding (3′ UTR) | chr12 | 51326219 | 51326283 | + |
| SEQ ID NO. 4 | 2332285 | FOXO6 | Non coding (3′ UTR) | chr1 | 41849216 | 41849252 | + |
| SEQ ID NO. 5 | 2478155 | MAP4K3 | Non coding (3′ UTR Antisense) | chr2 | 39476662 | 39476964 | + |
| SEQ ID NO. 6 | 3139092 | ARFGEF1 | Coding | chr8 | 68151048 | 68151077 | − |
| SEQ ID NO. 7 | 2789391 | LRBA | Coding | chr4 | 151791678 | 151791746 | − |
| SEQ ID NO. 8 | 3463598 | PPP1R12A | Coding | chr12 | 80190589 | 80190729 | − |
| SEQ ID NO. 9 | 2461837 | ARID4B | Coding | chr1 | 235392607 | 235392699 | − |
| SEQ ID NO. 10 | 2512364 | MARCH7 | Coding | chr2 | 160621140 | 160621185 | + |
| SEQ ID NO. 11 | 2364325 | HSD17B7 | Non coding (3′ UTR) | chr1 | 162782497 | 162782521 | + |
| SEQ ID NO. 12 | 3066770 | SYPL1 | Non coding (3′ UTR) | chr7 | 105731011 | 105731149 | − |
| SEQ ID NO. 13 | 2956442 | MUT | Non coding (3′ UTR) | chr6 | 49399033 | 49399073 | − |
| SEQ ID NO. 14 | 3005357 | CRCP | Non coding (3′ UTR) | chr7 | 65619506 | 65619539 | + |
| SEQ ID NO. 15 | 2651515 | MECOM | Non coding (Intronic Antisense) | chr3 | 169003654 | 169003734 | + |

Genomic characteristics of markers comprising the genomic classifier for predicting post-cystectomy bladder cancer recurrence.

TABLE 3

| | Relative Risk of Recurrence | |
|---|---|---|
| | Hazard ratio (95% CI) | P |
| GC | 1.23 (1.07-1.42) | 0.003 |
| Age <70 years | 1.00 (0.99-1.01) | 0.72 |
| Male Gender | 0.97 (0.89-1.06) | 0.46 |
| Caucasian Race | 0.95 (0.85-1.05) | 0.29 |
| Tumor Stage [reference: pT2] | | |
| pT1 | 1.02 (0.83-1.26) | 0.87 |
| pT3 | 1.23 (0.50-3.05) | 0.66 |
| pT4a | 3.37 (1.27-8.93) | 0.015 |
| Nodal Status N1-3 | 1.10 (1.03-1.19) | 0.006 |
| Lymphovascular Invasion present | 1.12 (1.03-1.20) | 0.005 |
| Adjuvant Chemotherapy administered | 1.03 (0.96-1.11) | 0.35 |

Abbreviations:
CI, confidence interval;
GC, genomic classifier.
Univariable associations of genomic and clinicopathologic features with risk of post-cystectomy bladder cancer recurrence in the validation cohort.

Prognostic performance of IBCNC and CC were comparable (standard-ROC AUC of 0.74 and 0.77, respectively) with the genomic model in the validation set. Again, the combined genomic-clinical models measured the highest performance of models tested (FIG. 1). Furthermore in survival ROC analysis (which factors in time to recurrence) the boost in performance with the combined models was more marked in terms of AUC (IBCNC versus G-IBCNC, 0.73 versus 0.82; CC versus G-CC, 0.78 versus 0.86). FIGS. 2B and C show that the combined genomic-clinical classifiers had the highest specificity and sensitivity across the widest range of cut-offs.

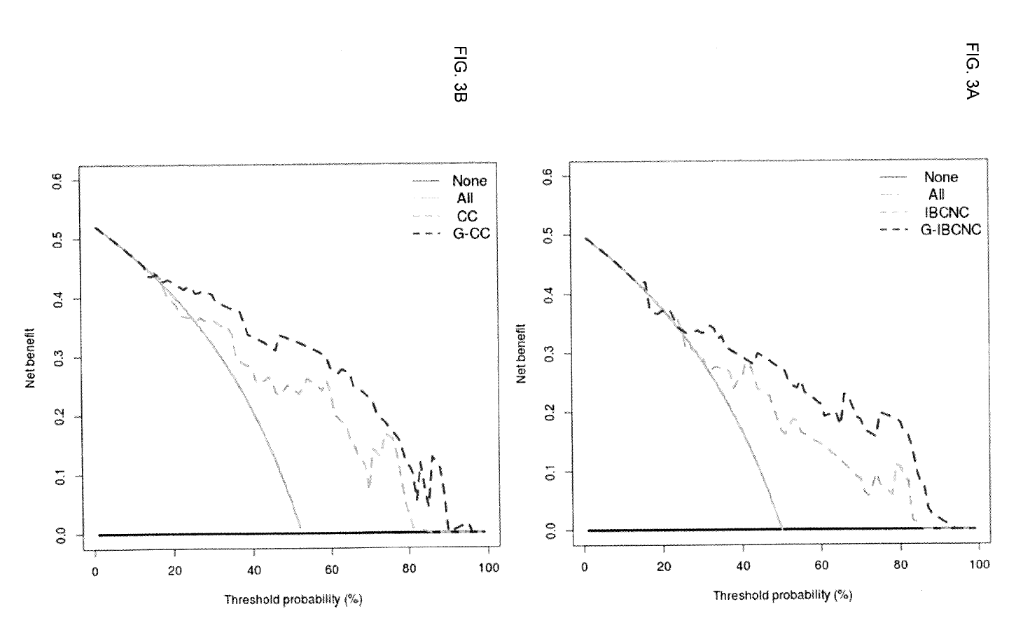

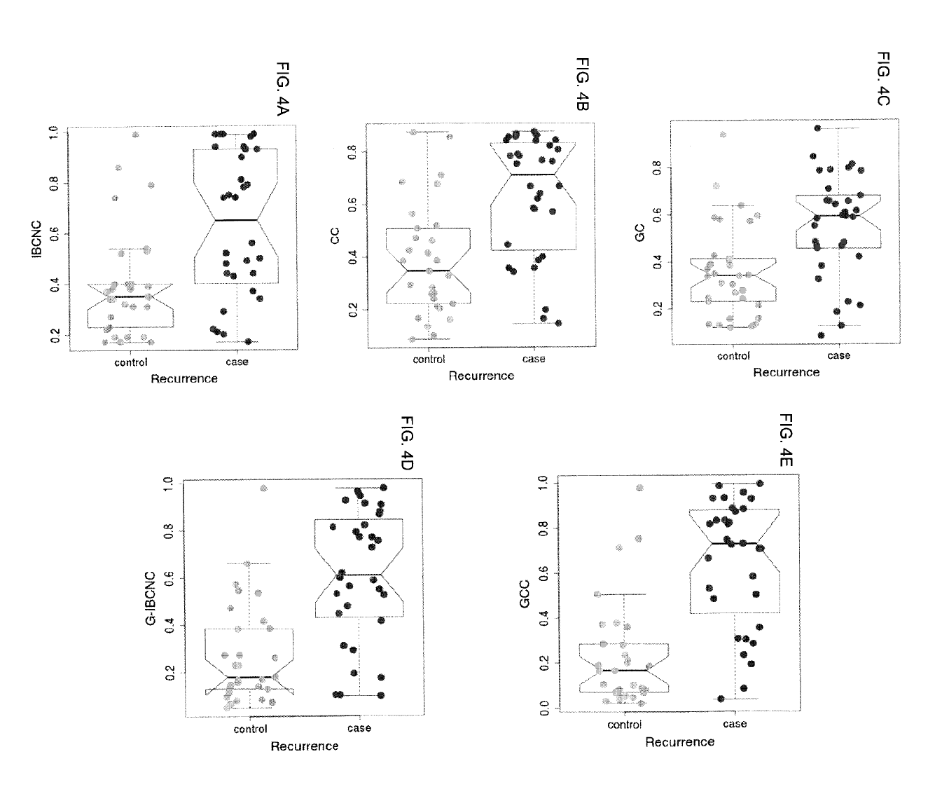

Multivariable analyses (Based on Cox proportional hazards analysis with ridge regression) showed that the independent prognostic significance of the combined genomic-clinical classifiers for predicting recurrence compared to clinical models alone (Table 4). Decision curve analysis showed a higher overall net benefit for genomic-based classifiers compared to clinical-only risk models (FIG. 3). Discrimination plots confirmed that while GC identified patients who recurred better than clinical models alone, the combined G-IBCNC and G-CC models were superior at this discrimination with G-CC performing the best (P=5.55×10⁻⁷; FIG. 4). G-CC was therefore considered the benchmark genomic-clinicopathologic classifier for further evaluation.

TABLE 4

Relative Risk of Recurrence

| | Hazard ratio (95% CI) | P |
|---|---|---|
| Model 1 | | |
| G-IBCNC | 1.18 (1.03-1.36) | 0.016 |
| IBCNC | 1.04 (0.90-1.20) | 0.62 |
| Model 2 | | |
| G-CC | 1.18 (1.04-1.33) | 0.008 |
| CC | 1.10 (0.92-1.31) | 0.30 |

Multivariable analysis comparing genomic-clinicopathologic versus clinical-only models in the validation cohort. Based on Cox proportional hazards analysis with ridge regression.

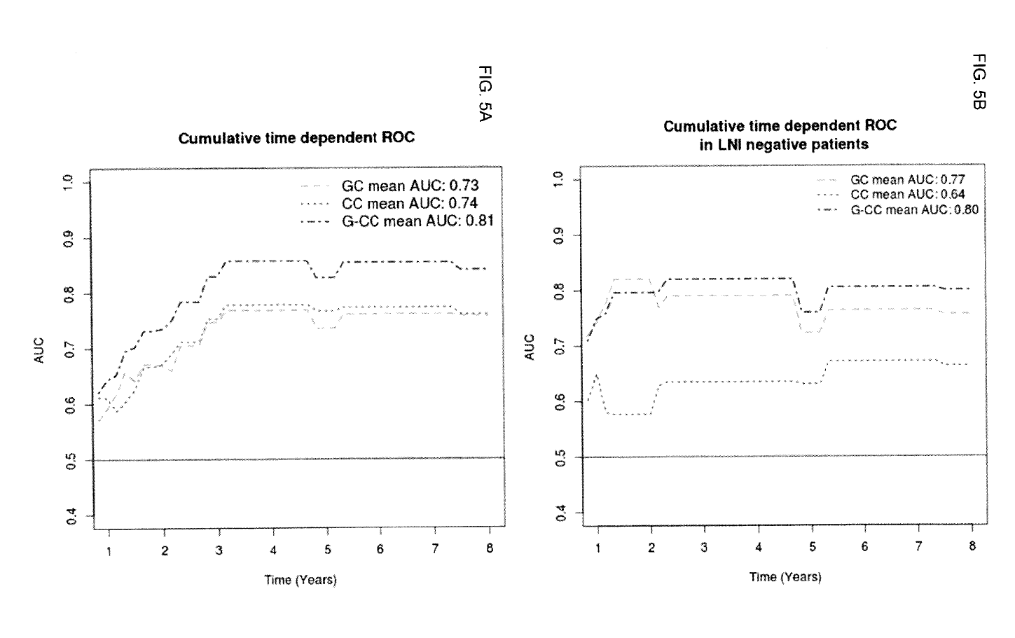

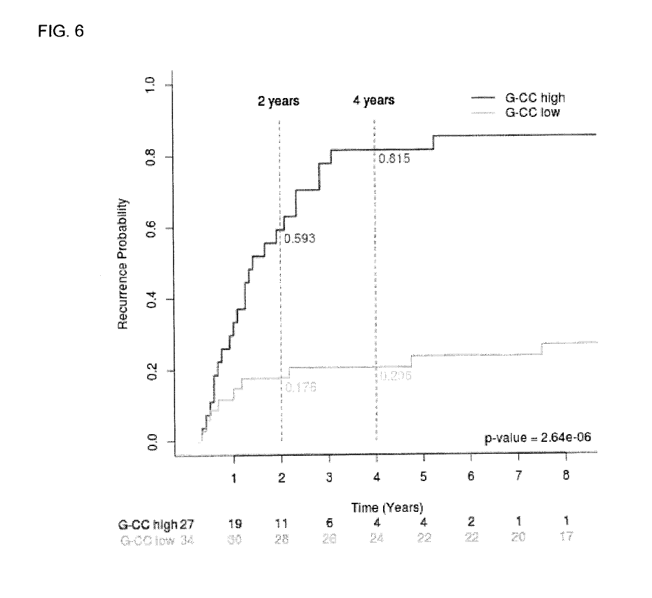

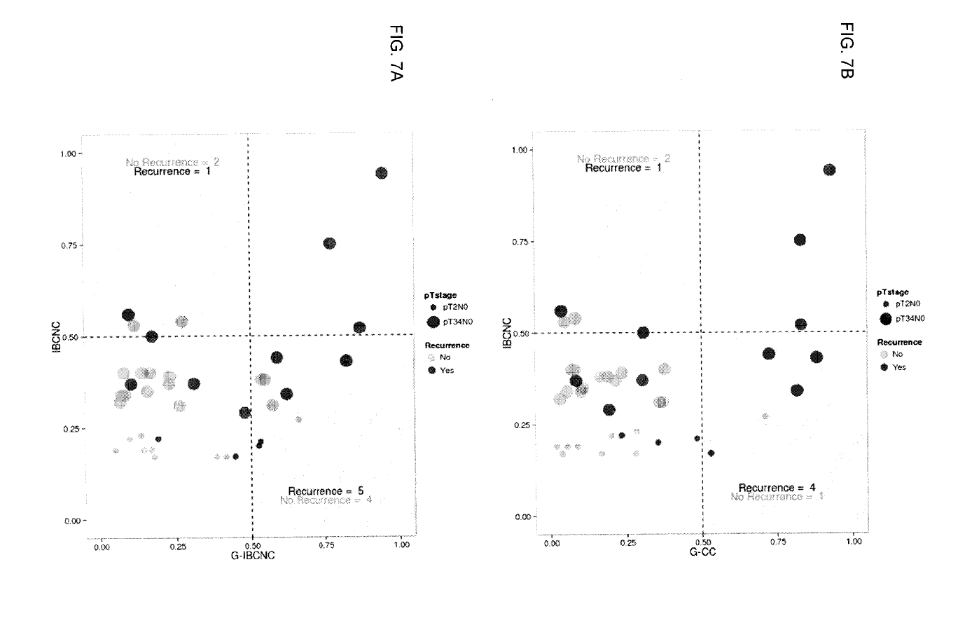

When survival-ROC AUCs were measured across a range of post-cystectomy intervals, G-CC showed consistently superior performance compared to CC and GC alone, indicating that the combined genomic-clinicopathologic classifier was the most prognostic indicator for RFS at any point in time after cystectomy (FIG. 5A). Validation set patients were then categorized by G-CC score and grouped as high-risk (G-CC score>0.5) or low-risk (G-CC score≦0.5). By competing risk analysis, high-risk patients had significantly elevated recurrence probabilities compared to low-risk patients (4-year probability: 81.5% versus 20.6%, respectively; P<0.001; FIG. 6). Patients were similarly risk-stratified based on IBCNC and G-IBCNC scores, and categorization by the “clinical-only” IBCNC nomogram was compared with genomic-clinical classifiers (FIG. 7). In FIG. 7, qQuadrants in grey represent situations where the combined genomic-clinical classifier reclassifies patients compared to the clinical classifier. Patients are colored by recurrence event and size of dots represent pathological tumor stage. Patients with no recurrence in the top left quadrant are reclassified correctly while patients with recurrence in the bottom right quadrant are reclassified correctly by the genomic-clinical classifier. Of the patients that are reclassified, the majority result in correct classification. Addition of genomic features to the IBCNC nomogram (as G-IBCNC) reclassified a total of 18 validation set patients into lower and higher predicted risk categories than those classified by IBCNC, of which 12 (66.7%) were reclassified correctly by outcome. Similarly, G-CC reclassified 12 patients that were originally risk stratified by IBCNC, of which 10 (83.3%) were reclassified correctly.

TABLE 5

| | Relative Risk of Recurrence | |
|---|---|---|
| | Hazard ratio (95% CI) | P |
| GC | 1.42 (1.11-1.81) | 0.005 |
| Age <70 years | 0.90 (0.78-1.03) | 0.12 |
| Male Gender | 0.00 (0.00-3.03) | 0.084 |
| Caucasian Race | 0.36 (0.11-1.20) | 0.097 |
| Tumor Stage [reference: pT2] | | |
| pT1 | 2.06 (0.15-27.73) | 0.59 |
| pT3 | 1.82 (0.61-5.42) | 0.29 |
| pT4a | 1.83 (0.50-6.69) | 0.36 |
| Nodal Status N1-3 | 9.80 (1.50-64.04) | 0.017 |
| Lymphovascular Invasion present | 2.74 (1.02-7.33) | 0.045 |
| Adjuvant Chemotherapy administered | 0.94 (0.41-2.13) | 0.88 |
| GC × Nodal Status* | 0.03 (0.00-0.72) | 0.030 |
| Gender × Age* | 1.12 (0.98-1.29) | 0.098 |

*Interaction term
Abbreviations:
CI, confidence interval;
GC, genomic classifier.
Multivariable associations of genomic and clinicopathologic features with risk of post-cystectomy bladder cancer recurrence in the validation cohort

### Nodal Status and Performance of Genomic-Based Classifier

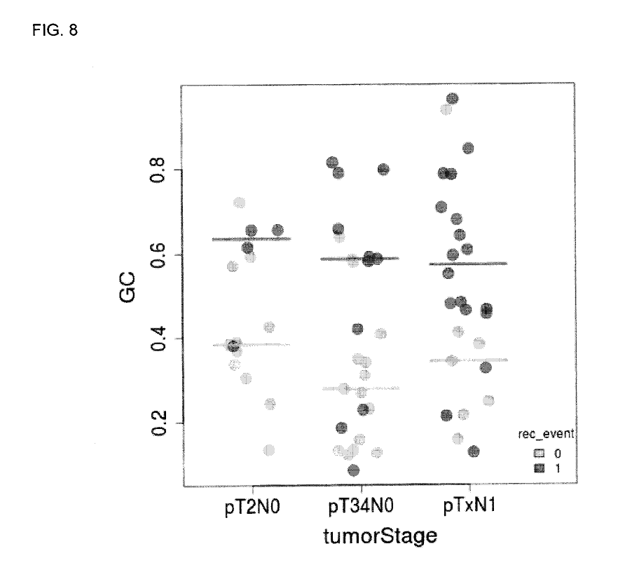

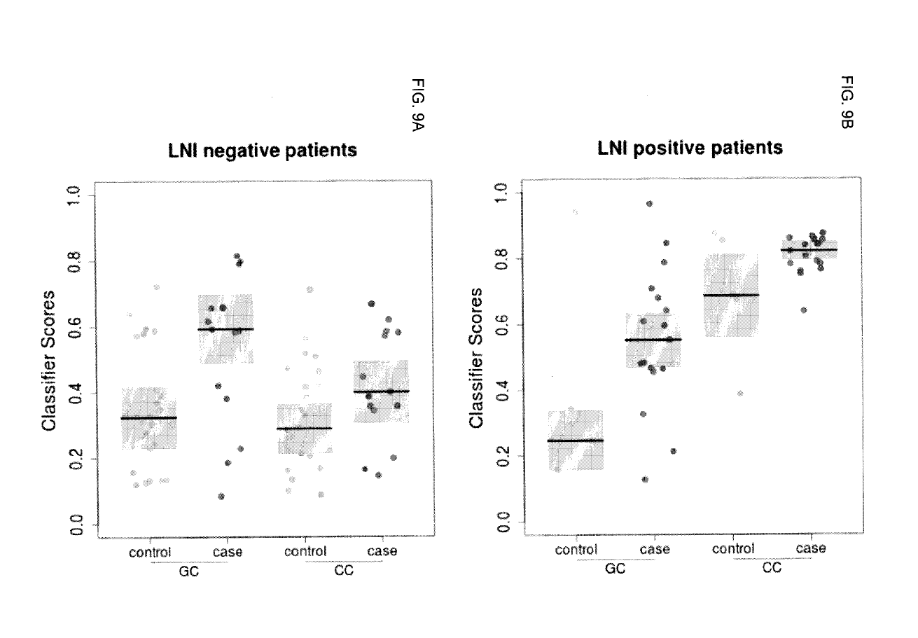

When validation cohort patients were categorized based on pathological stage, median GC scores were consistently higher in patients who recurred compared to those who did not (FIG. 8). By multivariable analysis, after adjusting for demographic, clinicopathologic and treatment covariates, GC remained prognostic for recurrence (P=0.005; Table 5). In this analysis, a significant interaction was noted between GC and nodal status (P=0.030) that prompted further exploration of GC score distribution based on nodal stage. When categorized by nodal status, GC scores were able to better discriminate validation cohort patients compared to CC (FIG. 9). As shown in FIG. 9, GC separates cases from controls in both LNI positive and LNI negative patients, as indicated by the non-overlapping 95% CI. In comparison, CC does not separate cases from controls as well. 95% CI calculated based on McGill et al. GC Wilcoxon rank-sum p-value for comparing case with control is 0.0078 compared CC with a p-value of 0.069 in the LNI negative patients. While in LNI positive patients, GC and CC has p-values of 0.16 and 0.41 respectively.

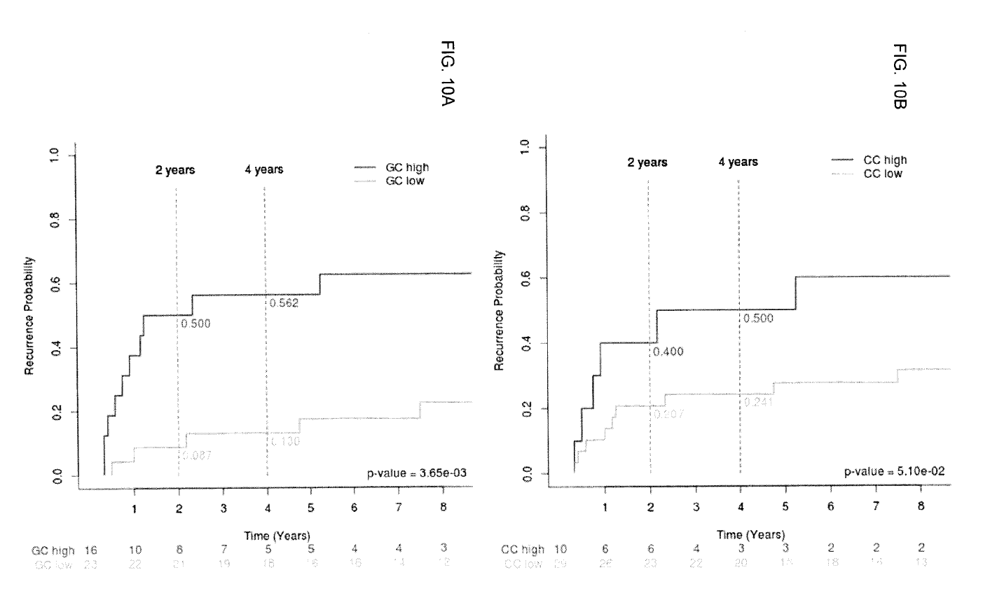

A subset analysis was then conducted on node-negative patients. These patients represent subjects who do not routinely receive adjuvant therapy. Assuming that higher risk patients have more to gain from therapeutic intensification in comparison to low risk, better identification of the subset of men most at risk by a more accurate risk prediction model could be beneficial. CC showed a trend towards significance in separating node-negative patients into risk groups (P=0.051). However, GC significantly stratified these patients based on recurrence (4-year probabilities: high 56.2% versus low 13%; P=0.004; FIG. 10). When measured across time, AUCs for genomic-based classifiers were consistently higher compared to CC alone in node-negative patients (FIG. 5B).

### Analysis of Prior Signatures and Biological Processes

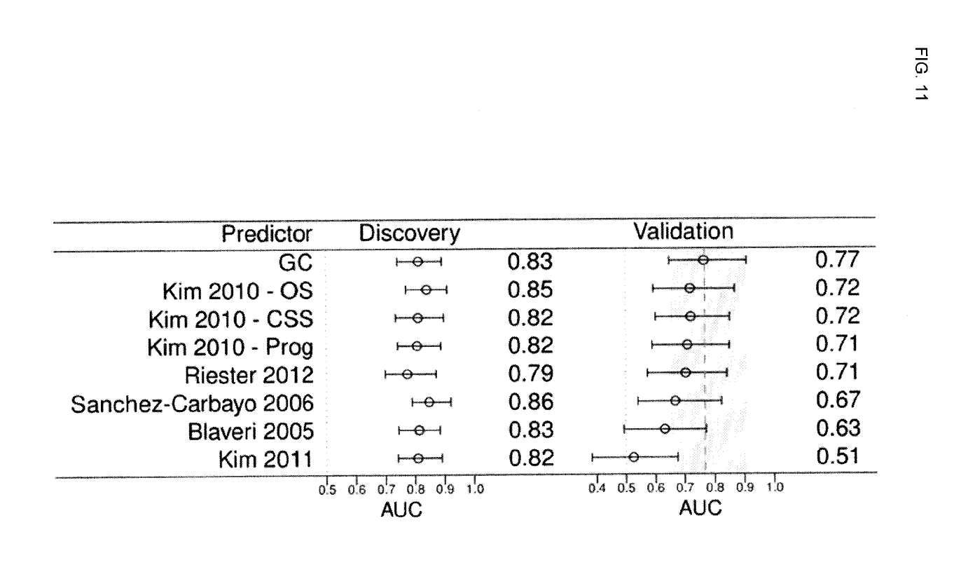

As the microarrays used to profile the study cohorts offer transcriptome-wide coverage including ability to interrogate entire lengths of protein-coding genes and non-coding RNAs, it was feasible to evaluate the performance of previously reported prognostic signatures for invasive UCB derived using early-generation microarrays. The performances of seven published post-cystectomy prognostic genomic signatures for muscle-invasive UCB were compared with the GC developed in this study (Table 6).(Blaveri et al. 2005; Sanchez-Carbayo et al. 2006; Kim et al. 2010; Kim et al. 2011; Riester et al. 2012) Genomic markers that comprised the individual signatures were mapped back to the respective Human Exon GeneChip features, and the performance of each signature was optimized in the discovery set as done originally for GC. Therefore, as expected, nearly all published signatures demonstrated high survival-ROC AUCs in the discovery set as that observed with GC. However, when these locked signatures were applied to the validation set (again, with bioinformaticians blinded to clinical variables) the AUC for each model decreased leaving the GC having the best validated performance (FIG. 11). Of the AUCs of published signatures that were modeled, lower bounds of the 95% CIs of 5/7 signatures were >0.50, suggesting a statistically significant ability to predict recurrence greater than random chance. The AUC of GC was 0.05 points higher than the best performing published signature, a 61-feature signature for post-cystectomy survival in patients with muscle-invasive and node-positive UCB reported by Kim et al, (Kim et al. 2010) which had an AUC of 0.72 (95% CI: 0.60-0.87).

TABLE 6

| | | | Signature | | | |
|---|---|---|---|---|---|---|
| | | | Size | | Survival-ROC | |
| | Discovery | | (no. of | | AUC (95% CI) | |
| | Cohort | | mappable | Prognostic | Discovery | Validation |
| Predictor Label* | n† | Discovery Profiling Array Platform | transcripts) | Endpoint | cohort | cohort |
| GC¹ | 133^{a} | Human Exon 1.0 ST GeneChip | 15 | Recurrence | 0.83 (0.75-0.91) | 0.77 (0.65-0.91) |
| | | (Affymetrix) | | free survival | | |
| Kim 2010 - OS² | 62^{a} | Human-6 v2.0 Expression Beadchip | 61 | Overall survival | 0.85 (0.78-0.92) | 0.72 (0.60-0.87) |
| | | (Illumina) | | | | |
| Kim 2010 - C5S² | 62^{b} | Human-6 v2.0 Expression Beadchip | 49 | Cancer specific | 0.82 (0.75-0.91) | 0.72 (0.60-0.85) |
| | | (Illumina) | | survival | | |
| Kim 2010 - Prog² | 62^{b} | Human-6 v2.0 Expression Beadchip | 44 | Progression | 0.82 (0.75-0.90) | 0.71 (0.59-0.85) |
| | | (Illumina) | | | | |
| Riester 2012³ | 78^{b} | Human Genome U133 Plus 2.0 | 20 | Overall survival | 0.79 (0.71-0.89) | 0.71 (0.58-0.83) |
| | | GeneChip (Affymetrix) | | | | |
| Sanchez-Carbayo 2006⁴ | 74^{c} | Human Genome U133A GeneChip | 85 | Overall survival | 0.85 (0.80-0.93) | 0.67 (0.54-0.83) |
| | | (Affymetrix) | | | | |
| Blaveri 2005⁵ | 40^{b} | Custom cDNA array | 24 | Overall survival | 0.83 (0.76-0.90) | 0.63 (0.50-0.77) |
| Kim 2011⁶ | 38^{b} | Human-6 v2.0 Expression Beadchip | 4 | Progression | 0.82 (0.75-0.90) | 0.51 (0.37-0.66) |
| | | (Illumina) | | | | |

Summary and performance metrics of prior prognostic signatures for high-risk bladder cancer in comparison with the genomic classifier.
†Patients with muscle-invasive or node-positive bladder cancer
^{a}Paraffin-embedded primary tumor specimens
^{b}Frozen primary tumor specimens

TABLE 7
Associations of markers comprising the genomic classifier with major Gene Ontology clusters
Biological interactions based on Human Signaling Network v5

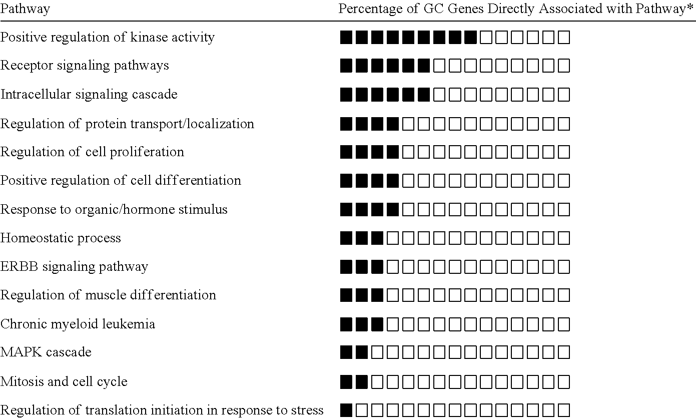

*Nine genes within GC directly associated with one or more pathways

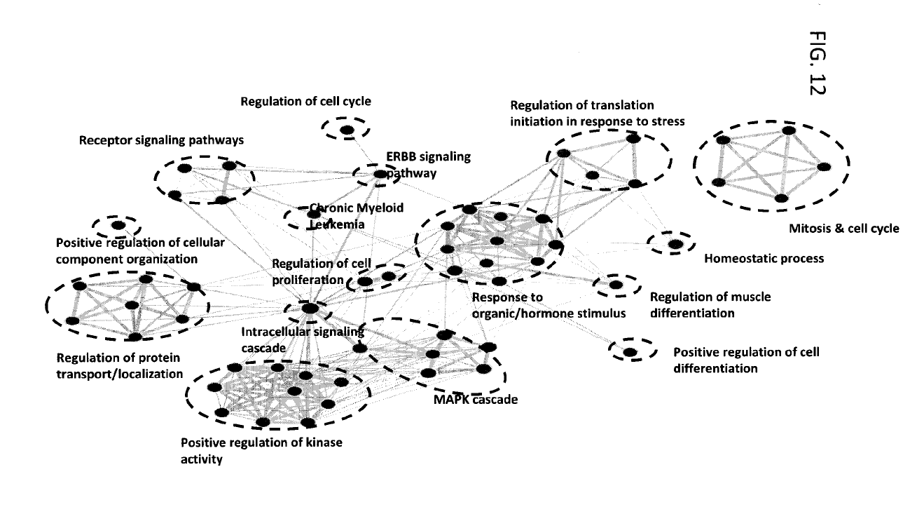

To analyze the biological relevance of markers included in GC, an interactome network-based gene set enrichment analysis of their 257 first-degree partners was performed (FIG. 12). Of these, 248 unique genes were identified to biologically interact with at least one of the 15 markers within GC. Biological processes and KEGG pathways were analyzed using the DAVID functional annotation tool, which indicated enrichment of GO terms associated with regulation of kinase activity and protein transport/localization. Additionally, GC signature genes were also well represented within GO terms associated with receptor and intracellular signaling pathways, and regulation of cell proliferation and differentiation (Table 7).

These results showed that whole transcriptome expression profiling could be utilized to stratify risk of disease recurrence in patients who have undergone radical cystectomy for muscle invasive bladder cancer. These results further showed that a 15-marker genomic classifier (GC) of the present invention surpasses the predictive power of previously reported clinical and genomic prognostic markers in bladder cancer. The results demonstrated that methods and markers of the present invention are useful for predicting risk for cancer recurrence. These results suggested that the methods and markers of the present invention would be useful for diagnosing, prognosing, determining the progression of cancer, or predicting benefit from therapy in a subject having cancer.

### Example 2

### Prognostic Value of Univariable and Combinations of Markers From a 15 Biomarker Panel for Muscle Invasive Bladder Cancer

The 15 markers identified in the genomic classifier in Example 1 (Table 2A, Table 2B) were assessed for their performance across a range of different metrics for the prediction of cancer recurrence within different subsets of clinical relevancy.

As shown in Table 8, these 15 markers, as univariable classifiers, were statistically significant in a pooled discovery and validation set (n=199; see Example 1) based on the Wilcoxon test (p-value <=0.05) for the Area under the ROC curve (AUC). The probeset ID number of the corresponding markers (Table 2B) as provided by Affymetrix (http://www.affymetrix.com/analysis/index.affx) was used to represent each marker.

Significance of the selected features was evidenced by multiple additional metrics (either in their raw values or as their associated P-value for assessment of statistical significance) including:
Sensitivity: proportion of the actual number of patients with the event that are correctly identified as such. Higher values indicate better performance.
Specificity: proportion of the actual number of patients without the event that are correctly identified as such. Higher values indicate better performance.
Area under the ROC curve (AUC): Corresponds to the area under the receiver operating characteristic curve, which plots the performance of a feature or classifier for all thresholds of sensitivity and specificity. Higher values indicate better performance. For survival AUC, performance was calculated for 4 years.
Accuracy: Proportion of patients correctly predicted. Higher values indicate better performance.
Positive Predictive Value (PPV): proportion of predicted events that are true events. Higher values indicate better performance.
Negative Predictive Value (NPV): proportion of predicted non-events that are true non-events. Higher values indicate better performance.
Median Fold Difference (MFD): the ratio of the median expression value for each group. Values away from 1 indicate better performance.
Univariable Analysis (UVA) odds ratio (OR): measures the effect size of the feature or classifier when partitioning the scores into low and high risk groups. For this metric, these groups are obtained by partitioning the set of samples into low and high risk expression values using the PAM clustering method. Values away from 1 indicate better performance.
Multivariable Analysis (MVA) odds ratio (OR): measures the independent prognostic ability of the feature or classifier when partitioning the values into low and high risk groups. For this metric, these groups are obtained by partitioning the set of samples into low and high risk expression values using the PAM clustering method. Values away from 1 indicate better performance.
UVA hazard ratio (HR): measures the ratio of the hazard rates when partitioning the expression values into low and high risk groups and incorporates the time to event through Cox proportionate hazard modeling. For this metric, these groups are obtained by partitioning the expression values into low and high risk using the PAM clustering method. Values away from 1 indicate better performance.
MVA hazard ratio (HR): measures the independent prognostic ability relative to other variables when partitioning the values into low and high risk groups and incorporates the time to event through Cox proportionate hazard modeling. For this metric, these groups are obtained by partitioning the expression values into low and high risk using the PAM clustering method. Values away from 1 indicate better performance.

Multivariable analyses included the following clinical data: age, gender, tumor stage, lymphovascular invasion, node status, race and adjuvant chemotherapy status (see Table 1).

The associated p-value provided for the metrics gives a measure of the statistical significance of the corresponding metric. The threshold of P-value <=0.05 is used as a way of defining those features that are statistically significant for the given metric and endpoint. The cut-off used for each marker is indicated. When dichotomization is necessary for a performance metric, the marker expression values are dichotomized using unsupervised clustering in the pamr package.

The superior performance provided by the 15 markers of the present invention is observed not only within the entire cohort used, but also when excluding the group of patients with pT2 (organ-confined) disease (Table 9) and within patients with negative lymph node involvement (Table 10). The former group represents patients that are prime candidates for therapy. The assessment of their likelihood of recurrence based on these 15 markers of the present invention can provide additional insight into the need for treatment. In contrast, patients with Lymph Node negative status represent a group that would be a candidate for observation; indications of aggressive disease based on these 15 markers of the present invention may be used to suggest treatment for these patients.

The 15 markers of the present invention are useful as univariable predictors; additionally, the combination of subsets of these 15 markers through a machine learning algorithm results in enhanced performance. As shown in Table 11, pairwise classifiers can result in an improved performance for the recurrence endpoint compared to their univariable counterparts, with all the classifiers listed presenting statistical significance based on, at least, Wilcox P-value. In Table 11, each classifier is described by the machine learning algorithm that combines the markers (column ‘classifier’) as well as the probeset ID number of the corresponding markers as provided by Affymetrix (http://www.affymetrix.com/analysis/index.affx).

These classifiers were trained and tuned in the discovery cohort (n=133) and the performance shown in Table 11 corresponds to that obtained for different metrics in the validation set (n=66). When dichotomization is necessary for a performance metric, the classifier scores are dichotomized using unsupervised clustering in the pamr package. The machine learning algorithms used are Naïve Bayes (NB), recursive Partitioning (Rpart), Support Vector Machines (SVMs), Random Forest (RF) and K Nearest Neighbors (KNN). These machine learning algorithms were executed with default parameters using packages rpart 4.1-0, HDclassif 1.2.2, randomForest 4.6-7, caret 5.15-61, cluster 1.14.3, e1071 1.6-1, class 7.3-5 in R. Each classifier generates a score between 0 and 1, except for SVM which generates a score from −∞ to +∞; in all cases, higher score values indicate higher probability of recurrence.

The improved performance of the classifiers built from combinations of subsets of the 15 markers is also observed within patients with negative lymph node involvement (Table 12), pT2 (organ-confined disease) patients (Table 13), patients with positive lymph node involvement (Table 14) as well as within the subset of patients that excludes the group with pT2 (organ-confined) disease (see Table 15). Patients with clinical low risk such as the first two groups (LNI- and pT2) can be candidates for treatment if aggressive disease based on the likelihood of recurrence obtained from the 15 markers is detected. In contrast, patients at a higher risk such as the second two groups may be spared unnecessary treatment if a low likelihood of recurrence is observed from the 15 markers of the present invention.

These results showed that the methods and markers of the present invention are useful for prognosing muscle invasive bladder cancer recurrence. These results further showed that the methods and markers of the present invention are useful for diagnosing, prognosing, determining the progression of cancer, or predicting benefit from therapy in a subject having cancer.

TABLE 8

| Affymetrix | | WILCOX | KS P- | | T-TEST | ACCU- | ACCURACY | SENSI- | SPECI- | | |
|---|---|---|---|---|---|---|---|---|---|---|---|
| Probeset ID | AUC | P-VALUE | VALUE | MFD | P-VALUE | RACY | P-VALUE | TIVITY | FICITY | PPV | NPV |
| 3326487 | 0.64 | 0.0008 | 0.0016 | 1.41 | 0.0004 | 0.59 | 0.0138 | 0.48 | 0.70 | 0.62 | 0.57 |
| 2458376 | 0.68 | 0.0000 | 0.0001 | 1.65 | 0.0000 | 0.63 | 0.0004 | 0.49 | 0.78 | 0.69 | 0.59 |
| 3414753 | 0.65 | 0.0003 | 0.0002 | 1.68 | 0.0003 | 0.64 | 0.0001 | 0.57 | 0.70 | 0.67 | 0.62 |
| 2332285 | 0.67 | 0.0000 | 0.0000 | 1.80 | 0.0000 | 0.63 | 0.0004 | 0.56 | 0.69 | 0.66 | 0.61 |
| 2478155 | 0.65 | 0.0002 | 0.0001 | 1.58 | 0.0003 | 0.60 | 0.0042 | 0.53 | 0.67 | 0.63 | 0.58 |
| 3139092 | 0.65 | 0.0003 | 0.0002 | 1.58 | 0.0013 | 0.59 | 0.0138 | 0.47 | 0.71 | 0.63 | 0.56 |
| 2789391 | 0.63 | 0.0012 | 0.0004 | 1.44 | 0.0007 | 0.63 | 0.0004 | 0.58 | 0.67 | 0.65 | 0.61 |
| 3463598 | 0.63 | 0.0016 | 0.0000 | 1.46 | 0.0016 | 0.64 | 0.0001 | 0.50 | 0.79 | 0.71 | 0.61 |
| 2461837 | 0.64 | 0.0007 | 0.0000 | 1.44 | 0.0017 | 0.59 | 0.0138 | 0.54 | 0.63 | 0.60 | 0.57 |
| 2512364 | 0.65 | 0.0004 | 0.0000 | 1.56 | 0.0003 | 0.63 | 0.0002 | 0.62 | 0.64 | 0.64 | 0.62 |
| 2364325 | 0.66 | 0.0001 | 0.0001 | 1.39 | 0.0002 | 0.60 | 0.0042 | 0.50 | 0.70 | 0.64 | 0.58 |
| 3066770 | 0.63 | 0.0016 | 0.0003 | 1.49 | 0.0024 | 0.62 | 0.0007 | 0.51 | 0.73 | 0.67 | 0.60 |
| 2956442 | 0.62 | 0.0039 | 0.0021 | 1.42 | 0.0049 | 0.62 | 0.0007 | 0.60 | 0.64 | 0.64 | 0.61 |
| 3005357 | 0.65 | 0.0002 | 0.0001 | 1.73 | 0.0004 | 0.61 | 0.0028 | 0.58 | 0.63 | 0.62 | 0.60 |
| 2651515 | 0.64 | 0.0006 | 0.0000 | 1.43 | 0.0004 | 0.65 | 0.0000 | 0.51 | 0.79 | 0.71 | 0.61 |
| Affymetrix | | KM P- | SURV- | UVA | UVA HR | UVA | UVA OR | MVA | MVA HR | MVA | MVA OR |
| Probeset ID | CUTOFF | VALUE | AUC | HR | P-VALUE | OR | P-VALUE | HR | P-VALUE | OR | P-VALUE |
| 3326487 | 4.78 | 0.0136 | 0.64 | 1.16 | 0.0004 | 1.27 | 0.0006 | 1.10 | 0.05 | 1.21 | 0.024 |
| 2458376 | 4.09 | 0.0001 | 0.67 | 1.21 | 0.0001 | 1.40 | 0.0001 | 1.11 | 0.08 | 1.29 | 0.012 |
| 3414753 | 4.44 | 0.0002 | 0.66 | 1.14 | 0.0011 | 1.26 | 0.0004 | 1.12 | 0.02 | 1.33 | 0.001 |
| 2332285 | 4.81 | 0.0004 | 0.66 | 1.17 | 0.0001 | 1.30 | 0.0001 | 1.11 | 0.03 | 1.28 | 0.003 |
| 2478155 | 4.75 | 0.0035 | 0.64 | 1.17 | 0.0007 | 1.30 | 0.0004 | 1.13 | 0.03 | 1.33 | 0.003 |
| 3139092 | 4.35 | 0.0077 | 0.67 | 1.15 | 0.0027 | 1.26 | 0.0018 | 1.12 | 0.03 | 1.29 | 0.006 |
| 2789391 | 5.54 | 0.0002 | 0.64 | 1.15 | 0.0008 | 1.23 | 0.0010 | 1.13 | 0.01 | 1.24 | 0.006 |
| 3463598 | 4.89 | 0.0000 | 0.64 | 1.16 | 0.0032 | 1.28 | 0.0021 | 1.08 | 0.23 | 1.22 | 0.047 |
| 2461837 | 4.27 | 0.0214 | 0.64 | 1.16 | 0.0031 | 1.27 | 0.0022 | 1.12 | 0.06 | 1.26 | 0.019 |
| 2512364 | 5.60 | 0.0001 | 0.65 | 1.16 | 0.0004 | 1.26 | 0.0004 | 1.14 | 0.01 | 1.31 | 0.001 |
| 2364325 | 5.87 | 0.0022 | 0.67 | 1.15 | 0.0005 | 1.27 | 0.0003 | 1.12 | 0.02 | 1.28 | 0.002 |
| 3066770 | 5.02 | 0.0012 | 0.64 | 1.13 | 0.0048 | 1.23 | 0.0030 | 1.08 | 0.15 | 1.20 | 0.032 |
| 2956442 | 4.75 | 0.0003 | 0.62 | 1.13 | 0.0071 | 1.21 | 0.0057 | 1.12 | 0.04 | 1.21 | 0.025 |
| 3005357 | 4.34 | 0.0018 | 0.66 | 1.15 | 0.0009 | 1.26 | 0.0006 | 1.13 | 0.01 | 1.30 | 0.003 |
| 2651515 | 4.21 | 0.0000 | 0.63 | 1.19 | 0.0006 | 1.35 | 0.0007 | 1.14 | 0.03 | 1.42 | 0.002 |

Performance of the 15 markers across different metrics for the Recurrence endpoint

TABLE 9

| Affymetrix | | WILCOX | KS P- | | T-TEST | | ACCURACY | | |
|---|---|---|---|---|---|---|---|---|---|
| Probeset ID | AUC | P-VALUE | VALUE | MFD | P-VALUE | ACCURACY | P-VALUE | SENSITIVITY | SPECIFICITY |
| 3326487 | 0.62 | 0.0166 | 0.0284 | 1.47 | 0.0074 | 0.58 | 0.5365 | 0.50 | 0.68 |
| 2458376 | 0.67 | 0.0006 | 0.0002 | 1.71 | 0.0021 | 0.61 | 0.2720 | 0.53 | 0.72 |
| 3414753 | 0.67 | 0.0010 | 0.0006 | 1.74 | 0.0004 | 0.64 | 0.0684 | 0.59 | 0.72 |
| 2332285 | 0.69 | 0.0002 | 0.0010 | 1.89 | 0.0001 | 0.64 | 0.0950 | 0.59 | 0.70 |
| 2478155 | 0.64 | 0.0058 | 0.0057 | 1.40 | 0.0061 | 0.61 | 0.2720 | 0.56 | 0.67 |
| 3139092 | 0.65 | 0.0030 | 0.0066 | 1.57 | 0.0075 | 0.57 | 0.6047 | 0.49 | 0.68 |
| 2789391 | 0.63 | 0.0089 | 0.0044 | 1.53 | 0.0039 | 0.63 | 0.1284 | 0.56 | 0.72 |
| 3463598 | 0.60 | 0.0467 | 0.0019 | 1.50 | 0.0307 | 0.63 | 0.1284 | 0.55 | 0.74 |
| 2461837 | 0.62 | 0.0207 | 0.0039 | 1.46 | 0.0440 | 0.56 | 0.6697 | 0.49 | 0.67 |
| 2512364 | 0.64 | 0.0075 | 0.0005 | 1.53 | 0.0038 | 0.61 | 0.2170 | 0.59 | 0.65 |
| 2364325 | 0.66 | 0.0014 | 0.0016 | 1.41 | 0.0015 | 0.60 | 0.3330 | 0.53 | 0.70 |
| 3066770 | 0.61 | 0.0330 | 0.0077 | 1.59 | 0.0320 | 0.61 | 0.2720 | 0.53 | 0.72 |
| 2956442 | 0.62 | 0.0149 | 0.0185 | 1.44 | 0.0172 | 0.63 | 0.1284 | 0.63 | 0.63 |
| 3005357 | 0.65 | 0.0025 | 0.0029 | 1.73 | 0.0032 | 0.59 | 0.3986 | 0.59 | 0.60 |
| 2651515 | 0.64 | 0.0073 | 0.0003 | 1.43 | 0.0069 | 0.64 | 0.0684 | 0.55 | 0.77 |
| Affymetrix | | | | KM P- | SURV- | | UVA HR | | UVA OR |
| Probeset ID | PPV | NPV | CUTOFF | VALUE | AUC | UVA HR | P-VALUE | UVA OR | P-VALUE |
| 3326487 | 0.68 | 0.50 | 4.84 | 0.0639 | 0.63 | 1.13 | 0.0128 | 1.24 | 0.0088 |
| 2458376 | 0.72 | 0.53 | 4.10 | 0.0087 | 0.66 | 1.14 | 0.0110 | 1.33 | 0.0030 |
| 3414753 | 0.74 | 0.56 | 4.51 | 0.0011 | 0.67 | 1.15 | 0.0030 | 1.33 | 0.0008 |
| 2332285 | 0.73 | 0.56 | 4.95 | 0.0040 | 0.67 | 1.15 | 0.0013 | 1.33 | 0.0003 |
| 2478155 | 0.70 | 0.53 | 4.88 | 0.0116 | 0.62 | 1.14 | 0.0213 | 1.29 | 0.0074 |
| 3139092 | 0.68 | 0.49 | 4.40 | 0.0574 | 0.67 | 1.13 | 0.0159 | 1.27 | 0.0090 |
| 2789391 | 0.73 | 0.55 | 5.83 | 0.0018 | 0.62 | 1.14 | 0.0056 | 1.24 | 0.0049 |
| 3463598 | 0.74 | 0.55 | 4.89 | 0.0027 | 0.61 | 1.11 | 0.0672 | 1.22 | 0.0327 |
| 2461837 | 0.67 | 0.49 | 4.75 | 0.0934 | 0.61 | 1.11 | 0.0647 | 1.20 | 0.0458 |
| 2512364 | 0.70 | 0.54 | 5.93 | 0.0098 | 0.64 | 1.14 | 0.0066 | 1.24 | 0.0047 |
| 2364325 | 0.71 | 0.52 | 5.87 | 0.0090 | 0.67 | 1.13 | 0.0067 | 1.27 | 0.0022 |
| 3066770 | 0.72 | 0.53 | 5.03 | 0.0147 | 0.62 | 1.09 | 0.0760 | 1.19 | 0.0340 |
| 2956442 | 0.70 | 0.55 | 4.89 | 0.0020 | 0.62 | 1.12 | 0.0342 | 1.22 | 0.0189 |
| 3005357 | 0.67 | 0.52 | 4.50 | 0.0327 | 0.65 | 1.13 | 0.0099 | 1.27 | 0.0041 |
| 2651515 | 0.77 | 0.56 | 4.36 | 0.0002 | 0.65 | 1.15 | 0.0151 | 1.31 | 0.0085 |

Performance of the 15 markers across different metrics for the Recurrence endpoint in the subset of patients excluding pT2 (organ-confined) disease.

TABLE 10

| Affymetrix | | WILCOX | KS P- | | T-TEST | | ACCURACY | | |
|---|---|---|---|---|---|---|---|---|---|
| Probeset ID | AUC | P-VALUE | VALUE | MFD | P-VALUE | ACCURACY | P-VALUE | SENSITIVITY | SPECIFICITY |
| 3326487 | 0.62 | 0.022 | 0.005 | 1.40 | 0.0027 | 0.67 | 0.26 | 0.49 | 0.77 |
| 2458376 | 0.68 | 0.001 | 0.006 | 1.47 | 0.0007 | 0.66 | 0.33 | 0.43 | 0.79 |
| 3414753 | 0.64 | 0.009 | 0.001 | 1.78 | 0.0072 | 0.67 | 0.26 | 0.57 | 0.72 |
| 2332285 | 0.67 | 0.001 | 0.001 | 1.63 | 0.0007 | 0.64 | 0.54 | 0.49 | 0.72 |
| 2478155 | 0.71 | 0.000 | 0.000 | 1.77 | 0.0001 | 0.65 | 0.39 | 0.60 | 0.68 |
| 3139092 | 0.67 | 0.001 | 0.002 | 1.59 | 0.0054 | 0.62 | 0.68 | 0.40 | 0.74 |
| 2789391 | 0.66 | 0.003 | 0.000 | 1.51 | 0.0020 | 0.67 | 0.21 | 0.60 | 0.72 |
| 3463598 | 0.62 | 0.020 | 0.001 | 1.50 | 0.0191 | 0.70 | 0.08 | 0.51 | 0.80 |
| 2461837 | 0.65 | 0.006 | 0.002 | 1.46 | 0.0119 | 0.60 | 0.80 | 0.60 | 0.61 |
| 2512364 | 0.69 | 0.000 | 0.000 | 1.65 | 0.0003 | 0.66 | 0.33 | 0.68 | 0.65 |
| 2364325 | 0.68 | 0.001 | 0.001 | 1.41 | 0.0009 | 0.65 | 0.39 | 0.51 | 0.73 |
| 3066770 | 0.64 | 0.010 | 0.002 | 1.52 | 0.0124 | 0.69 | 0.12 | 0.57 | 0.76 |
| 2956442 | 0.60 | 0.052 | 0.044 | 1.41 | 0.0442 | 0.62 | 0.68 | 0.57 | 0.65 |
| 3005357 | 0.66 | 0.003 | 0.006 | 1.82 | 0.0026 | 0.63 | 0.61 | 0.62 | 0.63 |
| 2651515 | 0.67 | 0.001 | 0.001 | 1.43 | 0.0019 | 0.61 | 0.74 | 0.55 | 0.65 |
| Affymetrix | | | | KM P- | SURV- | | UVA HR | | UVA OR |
| Probeset ID | PPV | NPV | CUTOFF | VALUE | AUC | UVA HR | P-VALUE | UVA OR | P-VALUE |
| 3326487 | 0.55 | 0.72 | 4.72 | 0.002 | 0.60 | 1.13 | 0.0027 | 1.28 | 0.004 |
| 2458376 | 0.54 | 0.71 | 4.06 | 0.006 | 0.67 | 1.13 | 0.0009 | 1.40 | 0.001 |
| 3414753 | 0.54 | 0.75 | 4.40 | 0.001 | 0.61 | 1.12 | 0.0119 | 1.24 | 0.009 |
| 2332285 | 0.50 | 0.71 | 4.82 | 0.021 | 0.63 | 1.13 | 0.0014 | 1.31 | 0.001 |
| 2478155 | 0.52 | 0.75 | 4.71 | 0.001 | 0.68 | 1.14 | 0.0002 | 1.40 | 0.000 |
| 3139092 | 0.48 | 0.69 | 4.40 | 0.056 | 0.69 | 1.13 | 0.0059 | 1.28 | 0.007 |
| 2789391 | 0.55 | 0.76 | 5.79 | 0.000 | 0.65 | 1.13 | 0.0026 | 1.28 | 0.003 |
| 3463598 | 0.60 | 0.74 | 4.90 | 0.000 | 0.61 | 1.12 | 0.0232 | 1.25 | 0.022 |
| 2461837 | 0.47 | 0.72 | 4.02 | 0.030 | 0.65 | 1.13 | 0.0120 | 1.27 | 0.014 |
| 2512364 | 0.52 | 0.78 | 5.61 | 0.000 | 0.69 | 1.13 | 0.0005 | 1.33 | 0.001 |
| 2364325 | 0.52 | 0.72 | 5.89 | 0.004 | 0.68 | 1.13 | 0.0016 | 1.30 | 0.001 |
| 3066770 | 0.57 | 0.76 | 4.83 | 0.000 | 0.64 | 1.12 | 0.0107 | 1.23 | 0.015 |
| 2956442 | 0.48 | 0.73 | 4.74 | 0.010 | 0.61 | 1.12 | 0.0518 | 1.18 | 0.046 |
| 3005357 | 0.49 | 0.74 | 4.37 | 0.005 | 0.66 | 1.13 | 0.0030 | 1.28 | 0.003 |
| 2651515 | 0.47 | 0.72 | 3.82 | 0.012 | 0.67 | 1.14 | 0.0010 | 1.39 | 0.003 |

Performance of the 15 markers across different metrics for the Recurrence endpoint in the subset of patients with negative lymph node involvement.

TABLE 11

| | | WILCOX | KS P- | T-TEST | ACCU- | ACCURACY | SENSI- | SPECI- | | | | KM P- | SURV- | UVA HR | UVA OR | MVA HR | MVA OR |
|---|---|---|---|---|---|---|---|---|---|---|---|---|---|---|---|---|---|
| CLASSIFIER | AUC | P-VALUE | VALUE | P-VALUE | RACY | P-VALUE | TIVITY | FICITY | PPV | NPV | CUTOFF | VALUE | AUC | P-VALUE | P-VALUE | P-VALUE | P-VALUE |
| nb:2478155 | 0.77 | 0.0001 | 0.0007 | 0.0001 | 0.73 | 0.0001 | 0.70 | 0.76 | 0.74 | 0.71 | 0.47 | 0.0002 | 0.77 | 0.0002 | 0.0007 | 0.001 | 0.002 |
| 2364325 | | | | | | | | | | | | | | | | | |
| knn:2458376 | 0.78 | 0.0001 | 0.0053 | 0.0001 | 0.70 | 0.0009 | 0.64 | 0.76 | 0.72 | 0.68 | 0.50 | 0.0019 | 0.75 | 0.0003 | 0.0004 | 0.010 | 0.003 |
| 2364325 | | | | | | | | | | | | | | | | | |
| nb:2332285 | 0.77 | 0.0001 | 0.0007 | 0.0006 | 0.70 | 0.0009 | 0.61 | 0.79 | 0.74 | 0.67 | 0.49 | 0.0015 | 0.78 | 0.0008 | 0.0018 | 0.006 | 0.003 |
| 3005357 | | | | | | | | | | | | | | | | | |
| rf:2478155 | 0.77 | 0.0001 | 0.0000 | 0.0000 | 0.76 | 0.0000 | 0.70 | 0.82 | 0.79 | 0.73 | 0.62 | 0.0000 | 0.77 | 0.0004 | 0.0004 | 0.008 | 0.001 |
| 2364325 | | | | | | | | | | | | | | | | | |
| nb:2458376 | 0.77 | 0.0001 | 0.0019 | 0.0002 | 0.68 | 0.0021 | 0.48 | 0.88 | 0.80 | 0.63 | 0.50 | 0.0018 | 0.77 | 0.0005 | 0.0011 | 0.021 | 0.004 |
| 3005357 | | | | | | | | | | | | | | | | | |
| svm:2478155 | 0.77 | 0.0001 | 0.0019 | 0.0001 | 0.71 | 0.0004 | 0.73 | 0.70 | 0.71 | 0.72 | −0.07 | 0.0006 | 0.79 | 0.0003 | 0.0004 | 0.001 | 0.001 |
| 3005357 | | | | | | | | | | | | | | | | | |
| rf:2458376 | 0.77 | 0.0001 | 0.0001 | 0.0001 | 0.68 | 0.0021 | 0.55 | 0.82 | 0.75 | 0.64 | 0.65 | 0.0027 | 0.77 | 0.0005 | 0.0005 | 0.007 | 0.001 |
| 2364325 | | | | | | | | | | | | | | | | | |
| svm:2332285 | 0.77 | 0.0001 | 0.0001 | 0.0004 | 0.67 | 0.0046 | 0.58 | 0.76 | 0.70 | 0.64 | −0.08 | 0.0075 | 0.76 | 0.0011 | 0.0013 | 0.023 | 0.008 |
| 3066770 | | | | | | | | | | | | | | | | | |
| knn:3414753 | 0.77 | 0.0001 | 0.0022 | 0.0004 | 0.67 | 0.0046 | 0.64 | 0.70 | 0.68 | 0.66 | 0.50 | 0.0072 | 0.74 | 0.0008 | 0.0011 | 0.007 | 0.002 |
| 3005357 | | | | | | | | | | | | | | | | | |
| svm:2332285 | 0.76 | 0.0002 | 0.0019 | 0.0002 | 0.73 | 0.0001 | 0.64 | 0.82 | 0.78 | 0.69 | 0.00 | 0.0002 | 0.77 | 0.0005 | 0.0007 | 0.007 | 0.003 |
| 2364325 | | | | | | | | | | | | | | | | | |
| nb:2332285 | 0.76 | 0.0002 | 0.0001 | 0.0012 | 0.68 | 0.0021 | 0.61 | 0.76 | 0.71 | 0.66 | 0.49 | 0.0033 | 0.76 | 0.0020 | 0.0029 | 0.010 | 0.005 |
| 3139092 | | | | | | | | | | | | | | | | | |
| nb:2458376 | 0.76 | 0.0002 | 0.0007 | 0.0002 | 0.65 | 0.0093 | 0.42 | 0.88 | 0.78 | 0.60 | 0.53 | 0.0122 | 0.76 | 0.0006 | 0.0013 | 0.084 | 0.011 |
| 3414753 | | | | | | | | | | | | | | | | | |
| knn:2458376 | 0.77 | 0.0002 | 0.0053 | 0.0001 | 0.68 | 0.0021 | 0.70 | 0.67 | 0.68 | 0.69 | 0.48 | 0.0039 | 0.75 | 0.0009 | 0.0005 | 0.011 | 0.001 |
| 3005357 | | | | | | | | | | | | | | | | | |
| nb:3414753 | 0.76 | 0.0002 | 0.0007 | 0.0013 | 0.64 | 0.0178 | 0.48 | 0.79 | 0.70 | 0.60 | 0.52 | 0.0157 | 0.74 | 0.0020 | 0.0032 | 0.048 | 0.011 |
| 2332285 | | | | | | | | | | | | | | | | | |
| nb:2478155 | 0.76 | 0.0002 | 0.0019 | 0.0003 | 0.68 | 0.0021 | 0.67 | 0.70 | 0.69 | 0.68 | 0.47 | 0.0037 | 0.73 | 0.0003 | 0.0013 | 0.001 | 0.002 |
| 3139092 | | | | | | | | | | | | | | | | | |
| svm:3414753 | 0.76 | 0.0002 | 0.0002 | 0.0002 | 0.65 | 0.0093 | 0.64 | 0.67 | 0.66 | 0.65 | −0.18 | 0.0201 | 0.76 | 0.0003 | 0.0009 | 0.011 | 0.002 |
| 2478155 | | | | | | | | | | | | | | | | | |
| knn:2478155 | 0.76 | 0.0003 | 0.0119 | 0.0003 | 0.67 | 0.0046 | 0.82 | 0.52 | 0.63 | 0.74 | 0.41 | 0.0059 | 0.76 | 0.0007 | 0.0011 | 0.002 | 0.001 |
| 3005357 | | | | | | | | | | | | | | | | | |
| nb:2478155 | 0.75 | 0.0003 | 0.0002 | 0.0002 | 0.70 | 0.0009 | 0.73 | 0.67 | 0.69 | 0.71 | 0.47 | 0.0014 | 0.73 | 0.0003 | 0.0007 | 0.004 | 0.002 |
| 2512364 | | | | | | | | | | | | | | | | | |
| nb:3414753 | 0.75 | 0.0003 | 0.0007 | 0.0004 | 0.67 | 0.0046 | 0.64 | 0.70 | 0.68 | 0.66 | 0.47 | 0.0072 | 0.77 | 0.0004 | 0.0013 | 0.004 | 0.001 |
| 3005357 | | | | | | | | | | | | | | | | | |
| svm:2332285 | 0.75 | 0.0003 | 0.0002 | 0.0002 | 0.71 | 0.0004 | 0.76 | 0.67 | 0.69 | 0.73 | 0.01 | 0.0006 | 0.77 | 0.0011 | 0.0008 | 0.003 | 0.001 |
| 3139092 | | | | | | | | | | | | | | | | | |
| nb:2458376 | 0.75 | 0.0003 | 0.0007 | 0.0002 | 0.68 | 0.0021 | 0.48 | 0.88 | 0.80 | 0.63 | 0.51 | 0.0018 | 0.72 | 0.0008 | 0.0011 | 0.063 | 0.007 |
| 2512364 | | | | | | | | | | | | | | | | | |
| rf:2332285 | 0.76 | 0.0004 | 0.0003 | 0.0002 | 0.70 | 0.0009 | 0.79 | 0.61 | 0.67 | 0.74 | 0.39 | 0.0015 | 0.78 | 0.0005 | 0.0008 | 0.001 | 0.001 |
| 3005357 | | | | | | | | | | | | | | | | | |
| nb:3414753 | 0.75 | 0.0004 | 0.0007 | 0.0004 | 0.73 | 0.0001 | 0.76 | 0.70 | 0.71 | 0.74 | 0.45 | 0.0002 | 0.77 | 0.0005 | 0.0014 | 0.011 | 0.004 |
| 2364325 | | | | | | | | | | | | | | | | | |
| nb:2332285 | 0.75 | 0.0004 | 0.0007 | 0.0007 | 0.70 | 0.0009 | 0.55 | 0.85 | 0.78 | 0.65 | 0.53 | 0.0007 | 0.76 | 0.0011 | 0.0020 | 0.015 | 0.006 |
| 2364325 | | | | | | | | | | | | | | | | | |
| svm:2332285 | 0.75 | 0.0004 | 0.0019 | 0.0012 | 0.70 | 0.0009 | 0.61 | 0.79 | 0.74 | 0.67 | −0.08 | 0.0017 | 0.75 | 0.0026 | 0.0027 | 0.024 | 0.008 |
| 2956442 | | | | | | | | | | | | | | | | | |
| svm:2458376 | 0.75 | 0.0004 | 0.0048 | 0.0002 | 0.70 | 0.0009 | 0.52 | 0.88 | 0.81 | 0.64 | 0.11 | 0.0010 | 0.74 | 0.0015 | 0.0009 | 0.048 | 0.003 |
| 3005357 | | | | | | | | | | | | | | | | | |
| rf:2332285 | 0.75 | 0.0004 | 0.0022 | 0.0003 | 0.67 | 0.0046 | 0.85 | 0.48 | 0.62 | 0.76 | 0.42 | 0.0093 | 0.74 | 0.0020 | 0.0011 | 0.089 | 0.005 |
| 2478155 | | | | | | | | | | | | | | | | | |
| rf:2458376 | 0.75 | 0.0004 | 0.0053 | 0.0002 | 0.67 | 0.0046 | 0.64 | 0.70 | 0.68 | 0.66 | 0.57 | 0.0078 | 0.77 | 0.0013 | 0.0008 | 0.002 | 0.000 |
| 3005357 | | | | | | | | | | | | | | | | | |
| nb:2332285 | 0.75 | 0.0004 | 0.0019 | 0.0010 | 0.70 | 0.0009 | 0.55 | 0.85 | 0.78 | 0.65 | 0.47 | 0.0007 | 0.73 | 0.0017 | 0.0030 | 0.083 | 0.011 |
| 2651515 | | | | | | | | | | | | | | | | | |
| nb:2458376 | 0.75 | 0.0004 | 0.0048 | 0.0004 | 0.68 | 0.0021 | 0.48 | 0.88 | 0.80 | 0.63 | 0.50 | 0.0039 | 0.75 | 0.0013 | 0.0020 | 0.031 | 0.009 |
| 3139092 | | | | | | | | | | | | | | | | | |
| nb:2478155 | 0.75 | 0.0004 | 0.0048 | 0.0004 | 0.68 | 0.0021 | 0.55 | 0.82 | 0.75 | 0.64 | 0.49 | 0.0008 | 0.71 | 0.0003 | 0.0015 | 0.004 | 0.004 |
| 3066770 | | | | | | | | | | | | | | | | | |
| nb:3005357 | 0.75 | 0.0004 | 0.0019 | 0.0004 | 0.67 | 0.0046 | 0.76 | 0.58 | 0.64 | 0.70 | 0.39 | 0.0063 | 0.77 | 0.0003 | 0.0017 | 0.013 | 0.002 |
| 2651515 | | | | | | | | | | | | | | | | | |
| nb:2364325 | 0.75 | 0.0004 | 0.0007 | 0.0004 | 0.74 | 0.0001 | 0.73 | 0.76 | 0.75 | 0.74 | 0.46 | 0.0001 | 0.76 | 0.0005 | 0.0012 | 0.001 | 0.001 |
| 3005357 | | | | | | | | | | | | | | | | | |
| nb:2512364 | 0.75 | 0.0005 | 0.0001 | 0.0003 | 0.73 | 0.0001 | 0.73 | 0.73 | 0.73 | 0.73 | 0.48 | 0.0002 | 0.73 | 0.0005 | 0.0010 | 0.005 | 0.002 |
| 2364325 | | | | | | | | | | | | | | | | | |
| nb:3139092 | 0.74 | 0.0005 | 0.0048 | 0.0008 | 0.68 | 0.0021 | 0.70 | 0.67 | 0.68 | 0.69 | 0.45 | 0.0031 | 0.76 | 0.0011 | 0.0022 | 0.002 | 0.004 |
| 2364325 | | | | | | | | | | | | | | | | | |
| nb:3139092 | 0.74 | 0.0005 | 0.0002 | 0.0007 | 0.67 | 0.0046 | 0.58 | 0.76 | 0.70 | 0.64 | 0.49 | 0.0013 | 0.77 | 0.0010 | 0.0020 | 0.001 | 0.001 |
| 3005357 | | | | | | | | | | | | | | | | | |
| rf:2478155 | 0.75 | 0.0005 | 0.0022 | 0.0003 | 0.70 | 0.0009 | 0.55 | 0.85 | 0.78 | 0.65 | 0.64 | 0.0006 | 0.72 | 0.0022 | 0.0012 | 0.085 | 0.003 |
| 3463598 | | | | | | | | | | | | | | | | | |
| knn:3414753 | 0.75 | 0.0006 | 0.0022 | 0.0003 | 0.71 | 0.0004 | 0.61 | 0.82 | 0.77 | 0.68 | 0.55 | 0.0005 | 0.75 | 0.0008 | 0.0009 | 0.026 | 0.003 |
| 2512364 | | | | | | | | | | | | | | | | | |
| nb:3414753 | 0.74 | 0.0006 | 0.0048 | 0.0011 | 0.64 | 0.0178 | 0.48 | 0.79 | 0.70 | 0.60 | 0.51 | 0.0177 | 0.76 | 0.0012 | 0.0029 | 0.008 | 0.004 |
| 3139092 | | | | | | | | | | | | | | | | | |
| svm:3139092 | 0.74 | 0.0006 | 0.0048 | 0.0005 | 0.70 | 0.0009 | 0.76 | 0.64 | 0.68 | 0.72 | −0.15 | 0.0013 | 0.76 | 0.0012 | 0.0014 | 0.001 | 0.003 |
| 2364325 | | | | | | | | | | | | | | | | | |
| knn:2458376 | 0.74 | 0.0006 | 0.0003 | 0.0005 | 0.71 | 0.0004 | 0.58 | 0.85 | 0.79 | 0.67 | 0.50 | 0.0001 | 0.73 | 0.0007 | 0.0019 | 0.025 | 0.005 |
| 2651515 | | | | | | | | | | | | | | | | | |
| rf:2458376 | 0.75 | 0.0006 | 0.0009 | 0.0006 | 0.70 | 0.0009 | 0.79 | 0.61 | 0.67 | 0.74 | 0.42 | 0.0036 | 0.75 | 0.0028 | 0.0017 | 0.044 | 0.005 |
| 2332285 | | | | | | | | | | | | | | | | | |
| nb:3414753 | 0.74 | 0.0006 | 0.0007 | 0.0004 | 0.73 | 0.0001 | 0.73 | 0.73 | 0.73 | 0.73 | 0.48 | 0.0002 | 0.74 | 0.0006 | 0.0012 | 0.018 | 0.003 |
| 2512364 | | | | | | | | | | | | | | | | | |
| svm:3414753 | 0.74 | 0.0006 | 0.0007 | 0.0003 | 0.73 | 0.0001 | 0.70 | 0.76 | 0.74 | 0.71 | −0.05 | 0.0002 | 0.74 | 0.0004 | 0.0011 | 0.008 | 0.003 |
| 2512364 | | | | | | | | | | | | | | | | | |
| rpart:2478155 | 0.70 | 0.0007 | 0.0119 | 0.0004 | 0.70 | 0.0009 | 0.88 | 0.52 | 0.64 | 0.81 | 0.47 | 0.0019 | 0.67 | 0.0047 | 0.0014 | 0.058 | 0.007 |
| 2956442 | | | | | | | | | | | | | | | | | |
| rpart:2478155 | 0.70 | 0.0007 | 0.0119 | 0.0004 | 0.70 | 0.0009 | 0.88 | 0.52 | 0.64 | 0.81 | 0.47 | 0.0019 | 0.67 | 0.0047 | 0.0014 | 0.058 | 0.007 |
| 3066770 | | | | | | | | | | | | | | | | | |
| nb:3066770 | 0.74 | 0.0007 | 0.0048 | 0.0008 | 0.65 | 0.0093 | 0.52 | 0.79 | 0.71 | 0.62 | 0.48 | 0.0054 | 0.76 | 0.0007 | 0.0021 | 0.003 | 0.003 |
| 3005357 | | | | | | | | | | | | | | | | | |
| rf:2332285 | 0.74 | 0.0007 | 0.0053 | 0.0005 | 0.70 | 0.0009 | 0.58 | 0.82 | 0.76 | 0.66 | 0.38 | 0.0005 | 0.72 | 0.0008 | 0.0018 | 0.064 | 0.014 |
| 2651515 | | | | | | | | | | | | | | | | | |
| knn:3463598 | 0.73 | 0.0008 | 0.0053 | 0.0004 | 0.71 | 0.0004 | 0.55 | 0.88 | 0.82 | 0.66 | 0.54 | 0.0001 | 0.74 | 0.0010 | 0.0016 | 0.029 | 0.007 |
| 3066770 | | | | | | | | | | | | | | | | | |
| svm:2364325 | 0.74 | 0.0008 | 0.0007 | 0.0003 | 0.73 | 0.0001 | 0.73 | 0.73 | 0.73 | 0.73 | 0.03 | 0.0002 | 0.78 | 0.0007 | 0.0009 | 0.001 | 0.001 |
| 3005357 | | | | | | | | | | | | | | | | | |
| rf:3414753 | 0.74 | 0.0008 | 0.0022 | 0.0008 | 0.65 | 0.0093 | 0.64 | 0.67 | 0.66 | 0.65 | 0.53 | 0.0201 | 0.75 | 0.0033 | 0.0017 | 0.035 | 0.003 |
| 2478155 | | | | | | | | | | | | | | | | | |
| knn:3066770 | 0.74 | 0.0008 | 0.0053 | 0.0014 | 0.65 | 0.0093 | 0.52 | 0.79 | 0.71 | 0.62 | 0.50 | 0.0054 | 0.74 | 0.0016 | 0.0028 | 0.006 | 0.003 |
| 3005357 | | | | | | | | | | | | | | | | | |
| nb:2512364 | 0.74 | 0.0008 | 0.0048 | 0.0004 | 0.70 | 0.0009 | 0.67 | 0.73 | 0.71 | 0.69 | 0.50 | 0.0007 | 0.74 | 0.0006 | 0.0011 | 0.002 | 0.001 |
| 3005357 | | | | | | | | | | | | | | | | | |
| nb:2512364 | 0.74 | 0.0008 | 0.0048 | 0.0008 | 0.70 | 0.0009 | 0.64 | 0.76 | 0.72 | 0.68 | 0.49 | 0.0007 | 0.72 | 0.0011 | 0.0020 | 0.016 | 0.005 |
| 3066770 | | | | | | | | | | | | | | | | | |
| svm:2458376 | 0.74 | 0.0008 | 0.0048 | 0.0014 | 0.65 | 0.0093 | 0.52 | 0.79 | 0.71 | 0.62 | 0.31 | 0.0219 | 0.70 | 0.0075 | 0.0030 | 0.124 | 0.007 |
| 3066770 | | | | | | | | | | | | | | | | | |
| knn:2478155 | 0.74 | 0.0008 | 0.0022 | 0.0003 | 0.71 | 0.0004 | 0.73 | 0.70 | 0.71 | 0.72 | 0.50 | 0.0005 | 0.74 | 0.0011 | 0.0009 | 0.022 | 0.002 |
| 2512364 | | | | | | | | | | | | | | | | | |
| nb:2478155 | 0.73 | 0.0009 | 0.0113 | 0.0005 | 0.67 | 0.0046 | 0.45 | 0.88 | 0.79 | 0.62 | 0.48 | 0.0023 | 0.72 | 0.0004 | 0.0022 | 0.029 | 0.002 |
| 2651515 | | | | | | | | | | | | | | | | | |
| nb:2512364 | 0.73 | 0.0009 | 0.0019 | 0.0008 | 0.64 | 0.0178 | 0.30 | 0.97 | 0.91 | 0.58 | 0.56 | 0.0013 | 0.73 | 0.0008 | 0.0027 | 0.058 | 0.004 |
| 2651515 | | | | | | | | | | | | | | | | | |
| nb:3326487 | 0.73 | 0.0009 | 0.0007 | 0.0066 | 0.64 | 0.0178 | 0.45 | 0.82 | 0.71 | 0.60 | 0.48 | 0.0180 | 0.71 | 0.0047 | 0.0116 | 0.079 | 0.059 |
| 2332285 | | | | | | | | | | | | | | | | | |
| rpart:2332285 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:2458376 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:2478155 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:2789391 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:3139092 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:3414753 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3463598 | | | | | | | | | | | | | | | | | |
| rpart:3463598 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 2364325 | | | | | | | | | | | | | | | | | |
| rpart:3463598 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 2512364 | | | | | | | | | | | | | | | | | |
| rpart:3463598 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3005357 | | | | | | | | | | | | | | | | | |
| rpart:3463598 | 0.70 | 0.0009 | 0.0119 | 0.0006 | 0.70 | 0.0009 | 0.55 | 0.85 | 0.78 | 0.65 | 0.53 | 0.0008 | 0.70 | 0.0014 | 0.0015 | 0.045 | 0.007 |
| 3066770 | | | | | | | | | | | | | | | | | |
| rf:2332285 | 0.74 | 0.0009 | 0.0009 | 0.0002 | 0.74 | 0.0001 | 0.67 | 0.82 | 0.79 | 0.71 | 0.47 | 0.0001 | 0.76 | 0.0007 | 0.0008 | 0.033 | 0.005 |
| 3463598 | | | | | | | | | | | | | | | | | |
| nb:2332285 | 0.73 | 0.0009 | 0.0007 | 0.0006 | 0.73 | 0.0001 | 0.64 | 0.82 | 0.78 | 0.69 | 0.51 | 0.0002 | 0.72 | 0.0012 | 0.0018 | 0.025 | 0.005 |
| 2512364 | | | | | | | | | | | | | | | | | |
| svm:2512364 | 0.73 | 0.0009 | 0.0001 | 0.0002 | 0.73 | 0.0001 | 0.73 | 0.73 | 0.73 | 0.73 | −0.20 | 0.0002 | 0.73 | 0.0006 | 0.0008 | 0.014 | 0.002 |
| 2364325 | | | | | | | | | | | | | | | | | |
| knn:2364325 | 0.74 | 0.0009 | 0.0009 | 0.0004 | 0.74 | 0.0001 | 0.76 | 0.73 | 0.74 | 0.75 | 0.50 | 0.0001 | 0.74 | 0.0010 | 0.0012 | 0.001 | 0.001 |
| 3005357 | | | | | | | | | | | | | | | | | |
| nb:2458376 | 0.73 | 0.0009 | 0.0048 | 0.0006 | 0.71 | 0.0004 | 0.52 | 0.91 | 0.85 | 0.65 | 0.47 | 0.0002 | 0.71 | 0.0011 | 0.0027 | 0.108 | 0.027 |
| 3066770 | | | | | | | | | | | | | | | | | |
| nb:2461837 | 0.73 | 0.0009 | 0.0019 | 0.0018 | 0.67 | 0.0046 | 0.58 | 0.76 | 0.70 | 0.64 | 0.49 | 0.0051 | 0.73 | 0.0021 | 0.0035 | 0.007 | 0.005 |
| 2364325 | | | | | | | | | | | | | | | | | |
| nb:3414753 | 0.73 | 0.0009 | 0.0113 | 0.0010 | 0.64 | 0.0178 | 0.39 | 0.88 | 0.76 | 0.59 | 0.50 | 0.0043 | 0.75 | 0.0010 | 0.0039 | 0.102 | 0.010 |
| 2651515 | | | | | | | | | | | | | | | | | |
| nb:3463598 | 0.73 | 0.0010 | 0.0048 | 0.0010 | 0.67 | 0.0046 | 0.55 | 0.79 | 0.72 | 0.63 | 0.50 | 0.0077 | 0.74 | 0.0012 | 0.0024 | 0.005 | 0.003 |
| 3005357 | | | | | | | | | | | | | | | | | |
| nb:2332285 | 0.73 | 0.0010 | 0.0007 | 0.0015 | 0.68 | 0.0021 | 0.61 | 0.76 | 0.71 | 0.66 | 0.46 | 0.0059 | 0.74 | 0.0020 | 0.0036 | 0.033 | 0.018 |
| 3066770 | | | | | | | | | | | | | | | | | |
| nb:2478155 | 0.73 | 0.0010 | 0.0007 | 0.0010 | 0.70 | 0.0009 | 0.70 | 0.70 | 0.70 | 0.70 | 0.46 | 0.0031 | 0.70 | 0.0009 | 0.0024 | 0.006 | 0.005 |
| 2461837 | | | | | | | | | | | | | | | | | |
| nb:2364325 | 0.73 | 0.0011 | 0.0019 | 0.0008 | 0.70 | 0.0009 | 0.52 | 0.88 | 0.81 | 0.64 | 0.50 | 0.0001 | 0.75 | 0.0008 | 0.0023 | 0.009 | 0.007 |
| 3066770 | | | | | | | | | | | | | | | | | |
| svm:2332285 | 0.73 | 0.0011 | 0.0048 | 0.0008 | 0.65 | 0.0093 | 0.70 | 0.61 | 0.64 | 0.67 | −0.20 | 0.0239 | 0.72 | 0.0038 | 0.0019 | 0.254 | 0.029 |
| 2461837 | | | | | | | | | | | | | | | | | |
| rpart:2332285 | 0.72 | 0.0011 | 0.0119 | 0.0009 | 0.64 | 0.0178 | 0.70 | 0.58 | 0.62 | 0.66 | 0.55 | 0.0376 | 0.71 | 0.0026 | 0.0020 | 0.073 | 0.016 |
| 2478155 | | | | | | | | | | | | | | | | | |
| knn:2512364 | 0.73 | 0.0011 | 0.0255 | 0.0006 | 0.67 | 0.0046 | 0.55 | 0.79 | 0.72 | 0.63 | 0.56 | 0.0057 | 0.71 | 0.0014 | 0.0016 | 0.039 | 0.004 |
| 3066770 | | | | | | | | | | | | | | | | | |
| knn:2458376 | 0.73 | 0.0012 | 0.0009 | 0.0001 | 0.73 | 0.0001 | 0.55 | 0.91 | 0.86 | 0.67 | 0.53 | 0.0002 | 0.73 | 0.0004 | 0.0005 | 0.127 | 0.007 |
| 3463598 | | | | | | | | | | | | | | | | | |
| rf:3463598 | 0.73 | 0.0012 | 0.0053 | 0.0006 | 0.65 | 0.0093 | 0.55 | 0.76 | 0.69 | 0.63 | 0.57 | 0.0172 | 0.74 | 0.0018 | 0.0015 | 0.004 | 0.001 |
| 3005357 | | | | | | | | | | | | | | | | | |
| svm:3066770 | 0.73 | 0.0012 | 0.0019 | 0.0007 | 0.67 | 0.0046 | 0.58 | 0.76 | 0.70 | 0.64 | 0.12 | 0.0058 | 0.77 | 0.0014 | 0.0016 | 0.003 | 0.002 |
| 3005357 | | | | | | | | | | | | | | | | | |
| nb:3139092 | 0.73 | 0.0013 | 0.0019 | 0.0007 | 0.68 | 0.0021 | 0.64 | 0.73 | 0.70 | 0.67 | 0.48 | 0.0021 | 0.75 | 0.0012 | 0.0019 | 0.004 | 0.002 |
| 2512364 | | | | | | | | | | | | | | | | | |
| knn:2364325 | 0.73 | 0.0013 | 0.0022 | 0.0004 | 0.70 | 0.0009 | 0.48 | 0.91 | 0.84 | 0.64 | 0.55 | 0.0001 | 0.73 | 0.0003 | 0.0016 | 0.004 | 0.002 |
| 3066770 | | | | | | | | | | | | | | | | | |
| rf:2478155 | 0.73 | 0.0013 | 0.0053 | 0.0009 | 0.67 | 0.0046 | 0.67 | 0.67 | 0.67 | 0.67 | 0.44 | 0.0106 | 0.71 | 0.0026 | 0.0021 | 0.015 | 0.002 |
| 2789391 | | | | | | | | | | | | | | | | | |
| svm:3414753 | 0.73 | 0.0013 | 0.0048 | 0.0010 | 0.71 | 0.0004 | 0.64 | 0.79 | 0.75 | 0.68 | −0.01 | 0.0004 | 0.73 | 0.0028 | 0.0020 | 0.030 | 0.006 |
| 2332285 | | | | | | | | | | | | | | | | | |
| nb:2458376 | 0.73 | 0.0014 | 0.0019 | 0.0013 | 0.68 | 0.0021 | 0.58 | 0.79 | 0.73 | 0.65 | 0.40 | 0.0042 | 0.73 | 0.0030 | 0.0053 | 0.195 | 0.024 |
| 2651515 | | | | | | | | | | | | | | | | | |
| rpart:2512364 | 0.70 | 0.0014 | 0.0119 | 0.0010 | 0.70 | 0.0009 | 0.76 | 0.64 | 0.68 | 0.72 | 0.48 | 0.0025 | 0.69 | 0.0041 | 0.0018 | 0.057 | 0.006 |
| 2364325 | | | | | | | | | | | | | | | | | |
| svm:2512364 | 0.72 | 0.0015 | 0.0048 | 0.0020 | 0.71 | 0.0004 | 0.67 | 0.76 | 0.73 | 0.69 | 0.18 | 0.0004 | 0.73 | 0.0039 | 0.0037 | 0.034 | 0.004 |
| 3066770 | | | | | | | | | | | | | | | | | |
| rf:2332285 | 0.73 | 0.0015 | 0.0022 | 0.0006 | 0.71 | 0.0004 | 0.64 | 0.79 | 0.75 | 0.68 | 0.50 | 0.0003 | 0.74 | 0.0012 | 00015 | 0.016 | 0.004 |
| 2364325 | | | | | | | | | | | | | | | | | |
| knn:2789391 | 0.73 | 0.0015 | 0.0255 | 0.0043 | 0.65 | 0.0093 | 0.52 | 0.79 | 0.71 | 0.62 | 0.50 | 0.0081 | 0.68 | 0.0057 | 0.0067 | 0.007 | 0.003 |
| 2364325 | | | | | | | | | | | | | | | | | |
| rpart:2332285 | 0.70 | 0.0015 | 0.0119 | 0.0010 | 0.70 | 0.0009 | 0.73 | 0.67 | 0.69 | 0.71 | 0.51 | 0.0010 | 0.72 | 0.0019 | 0.0019 | 0.002 | 0.002 |
| 2364325 | | | | | | | | | | | | | | | | | |
| rpart:2364325 | 0.70 | 0.0015 | 0.0119 | 0.0010 | 0.70 | 0.0009 | 0.73 | 0.67 | 0.69 | 0.71 | 0.51 | 0.0010 | 0.72 | 0.0019 | 0.0019 | 0.002 | 0.002 |
| 2956442 | | | | | | | | | | | | | | | | | |
| rpart:2364325 | 0.70 | 0.0015 | 0.0119 | 0.0010 | 0.70 | 0.0009 | 0.73 | 0.67 | 0.69 | 0.71 | 0.51 | 0.0010 | 0.72 | 0.0019 | 0.0019 | 0.002 | 0.002 |
| 3066770 | | | | | | | | | | | | | | | | | |
| nb:2478155 | 0.72 | 0.0015 | 0.0048 | 0.0006 | 0.67 | 0.0046 | 0.55 | 0.79 | 0.72 | 0.63 | 0.51 | 0.0065 | 0.71 | 0.0007 | 0.0018 | 0.009 | 0.006 |
| 3463598 | | | | | | | | | | | | | | | | | |
| svm:3139092 | 0.72 | 0.0015 | 0.0019 | 0.0073 | 0.64 | 0.0178 | 0.73 | 0.55 | 0.62 | 0.67 | −0.03 | 0.0238 | 0.77 | 0.0078 | 0.0106 | 0.001 | 0.001 |
| 2956442 | | | | | | | | | | | | | | | | | |
| rf:2458376 | 0.73 | 0.0015 | 0.0255 | 0.0012 | 0.67 | 0.0046 | 0.55 | 0.79 | 0.72 | 0.63 | 0.58 | 0.0092 | 0.75 | 0.0044 | 0.0025 | 0.051 | 0.004 |
| 3414753 | | | | | | | | | | | | | | | | | |
| knn:2332285 | 0.72 | 0.0016 | 0.0119 | 0.0034 | 0.67 | 0.0046 | 0.42 | 0.91 | 0.82 | 0.61 | 0.64 | 0.0014 | 0.75 | 0.0047 | 0.0066 | 0.074 | 0.010 |
| 3463598 | | | | | | | | | | | | | | | | | |
| knn:3463598 | 0.72 | 0.0016 | 0.0053 | 0.0014 | 0.65 | 0.0093 | 0.42 | 0.88 | 0.78 | 0.60 | 0.59 | 0.0201 | 0.73 | 0.0041 | 0.0035 | 0.174 | 0.011 |
| 2512364 | | | | | | | | | | | | | | | | | |
| knn:3463598 | 0.73 | 0.0016 | 0.0022 | 0.0006 | 0.68 | 0.0021 | 0.55 | 0.82 | 0.75 | 0.64 | 0.53 | 0.0027 | 0.76 | 0.0017 | 0.0016 | 0.012 | 0.002 |
| 2364325 | | | | | | | | | | | | | | | | | |
| nb:3326487 | 0.72 | 0.0016 | 0.0048 | 0.0056 | 0.56 | 0.1945 | 0.30 | 0.82 | 0.63 | 0.54 | 0.50 | 0.2816 | 0.74 | 0.0023 | 0.0105 | 0.018 | 0.019 |
| 3005357 | | | | | | | | | | | | | | | | | |
| svm:2458376 | 0.72 | 0.0016 | 0.0002 | 0.0025 | 0.71 | 0.0004 | 0.61 | 0.82 | 0.77 | 0.68 | 0.10 | 0.0007 | 0.70 | 0.0036 | 0.0051 | 0.127 | 0.004 |
| 2512364 | | | | | | | | | | | | | | | | | |
| svm:2458376 | 0.72 | 0.0016 | 0.0048 | 0.0009 | 0.70 | 0.0009 | 0.64 | 0.76 | 0.72 | 0.68 | −0.18 | 0.0043 | 0.73 | 0.0028 | 0.0022 | 0.095 | 0.009 |
| 2651515 | | | | | | | | | | | | | | | | | |
| rf:2458376 | 0.73 | 0.0017 | 0.0009 | 0.0003 | 0.74 | 0.0001 | 0.55 | 0.94 | 0.90 | 0.67 | 0.63 | 0.0000 | 0.74 | 0.0011 | 0.0011 | 0.052 | 0.005 |
| 3463598 | | | | | | | | | | | | | | | | | |
| nb:2332285 | 0.72 | 0.0017 | 0.0007 | 0.0016 | 0.74 | 0.0001 | 0.67 | 0.82 | 0.79 | 0.71 | 0.47 | 0.0001 | 0.73 | 0.0028 | 0.0038 | 0.056 | 0.014 |
| 3463598 | | | | | | | | | | | | | | | | | |
| nb:2364325 | 0.72 | 0.0018 | 0.0113 | 0.0012 | 0.64 | 0.0178 | 0.45 | 0.82 | 0.71 | 0.60 | 0.48 | 0.0152 | 0.75 | 0.0012 | 0.0037 | 0.036 | 0.006 |
| 2651515 | | | | | | | | | | | | | | | | | |
| nb:3463598 | 0.72 | 0.0018 | 0.0002 | 0.0011 | 0.73 | 0.0001 | 0.64 | 0.82 | 0.78 | 0.69 | 0.52 | 0.0002 | 0.72 | 0.0018 | 0.0026 | 0.031 | 0.006 |
| 2512364 | | | | | | | | | | | | | | | | | |
| svm:3139092 | 0.72 | 0.0018 | 0.0048 | 0.0009 | 0.70 | 0.0009 | 0.73 | 0.67 | 0.69 | 0.71 | 0.19 | 0.0008 | 0.77 | 0.0015 | 0.0023 | 0.000 | 0.001 |
| 3005357 | | | | | | | | | | | | | | | | | |
| knn:2458376 | 0.72 | 0.0018 | 0.0053 | 0.0003 | 0.68 | 0.0021 | 0.48 | 0.88 | 0.80 | 0.63 | 0.55 | 0.0018 | 0.72 | 0.0017 | 0.0011 | 0.125 | 0.006 |
| 2512364 | | | | | | | | | | | | | | | | | |
| rf:2478155 | 0.72 | 0.0019 | 0.0022 | 0.0001 | 0.71 | 0.0004 | 0.70 | 0.73 | 0.72 | 0.71 | 0.66 | 0.0003 | 0.76 | 0.0005 | 0.0004 | 0.001 | 0.001 |
| 3005357 | | | | | | | | | | | | | | | | | |
| knn:2478155 | 0.72 | 0.0019 | 0.0255 | 0.0012 | 0.65 | 0.0093 | 0.70 | 0.61 | 0.64 | 0.67 | 0.50 | 0.0176 | 0.71 | 0.0043 | 0.0030 | 0.003 | 0.001 |
| 3139092 | | | | | | | | | | | | | | | | | |
| knn:3414753 | 0.72 | 0.0019 | 0.0119 | 0.0008 | 0.70 | 0.0009 | 0.58 | 0.82 | 0.76 | 0.66 | 0.50 | 0.0007 | 0.72 | 0.0011 | 0.0020 | 0.015 | 0.005 |
| 2789391 | | | | | | | | | | | | | | | | | |
| nb:2461837 | 0.72 | 0.0019 | 0.0019 | 0.0037 | 0.65 | 0.0093 | 0.58 | 0.73 | 0.68 | 0.63 | 0.46 | 0.0088 | 0.72 | 0.0042 | 0.0057 | 0.008 | 0.005 |
| 3005357 | | | | | | | | | | | | | | | | | |
| nb:3139092 | 0.72 | 0.0019 | 0.0019 | 0.0032 | 0.64 | 0.0178 | 0.48 | 0.79 | 0.70 | 0.60 | 0.49 | 0.0117 | 0.74 | 0.0030 | 0.0059 | 0.008 | 0.008 |
| 3066770 | | | | | | | | | | | | | | | | | |
| svm:3414753 | 0.72 | 0.0019 | 0.0113 | 0.0016 | 0.68 | 0.0021 | 0.76 | 0.61 | 0.66 | 0.71 | −0.01 | 0.0045 | 0.78 | 0.0043 | 0.0029 | 0.006 | 0.002 |
| 3139092 | | | | | | | | | | | | | | | | | |
| knn:2461837 | 0.72 | 0.0020 | 0.0022 | 0.0005 | 0.73 | 0.0001 | 0.76 | 0.70 | 0.71 | 0.74 | 0.44 | 0.0005 | 0.69 | 0.0014 | 0.0012 | 0.002 | 0.001 |
| 3005357 | | | | | | | | | | | | | | | | | |
| rf:3463598 | 0.72 | 0.0020 | 0.0022 | 0.0005 | 0.70 | 0.0009 | 0.58 | 0.82 | 0.76 | 0.66 | 0.57 | 0.0013 | 0.75 | 0.0015 | 0.0014 | 0.032 | 0.003 |
| 2364325 | | | | | | | | | | | | | | | | | |
| svm:2364325 | 0.72 | 0.0021 | 0.0019 | 0.0008 | 0.70 | 0.0009 | 0.52 | 0.88 | 0.81 | 0.64 | 0.17 | 0.0001 | 0.73 | 0.0013 | 0.0019 | 0.015 | 0.005 |
| 3066770 | | | | | | | | | | | | | | | | | |
| rpart:2458376 | 0.71 | 0.0021 | 0.0511 | 0.0014 | 0.67 | 0.0046 | 0.73 | 0.61 | 0.65 | 0.69 | 0.39 | 0.0144 | 0.72 | 0.0018 | 0.0029 | 0.048 | 0.007 |
| 2651515 | | | | | | | | | | | | | | | | | |
| knn:2458376 | 0.72 | 0.0022 | 0.0255 | 0.0013 | 0.68 | 0.0021 | 0.64 | 0.73 | 0.70 | 0.67 | 0.50 | 0.0035 | 0.70 | 0.0047 | 0.0033 | 0.302 | 0.019 |
| 3066770 | | | | | | | | | | | | | | | | | |
| nb:2478155 | 0.72 | 0.0022 | 0.0113 | 0.0023 | 0.64 | 0.0178 | 0.45 | 0.82 | 0.71 | 0.60 | 0.49 | 0.0097 | 0.69 | 0.0015 | 0.0047 | 0.008 | 0.004 |
| 2789391 | | | | | | | | | | | | | | | | | |
| rpart:2458376 | 0.71 | 0.0023 | 0.0119 | 0.0093 | 0.64 | 0.0178 | 0.55 | 0.73 | 0.67 | 0.62 | 0.57 | 0.0222 | 0.70 | 0.0115 | 0.0118 | 0.017 | 0.003 |
| 2956442 | | | | | | | | | | | | | | | | | |
| rf:3066770 | 0.72 | 0.0023 | 0.0022 | 0.0010 | 0.67 | 0.0046 | 0.64 | 0.70 | 0.68 | 0.66 | 0.53 | 0.0051 | 0.75 | 0.0013 | 0.0021 | 0.002 | 0.002 |
| 3005357 | | | | | | | | | | | | | | | | | |
| knn:2956442 | 0.72 | 0.0023 | 0.0255 | 0.0073 | 0.62 | 0.0320 | 0.52 | 0.73 | 0.65 | 0.60 | 0.47 | 0.0407 | 0.68 | 0.0101 | 0.0108 | 0.029 | 0.005 |
| 2651515 | | | | | | | | | | | | | | | | | |
| knn:2478155 | 0.72 | 0.0023 | 0.0053 | 0.0004 | 0.71 | 0.0004 | 0.55 | 0.88 | 0.82 | 0.66 | 0.52 | 0.0001 | 0.71 | 0.0007 | 0.0014 | 0.033 | 0.002 |
| 2651515 | | | | | | | | | | | | | | | | | |
| nb:3326487 | 0.71 | 0.0024 | 0.0505 | 0.0030 | 0.59 | 0.0876 | 0.36 | 0.82 | 0.67 | 0.56 | 0.48 | 0.1071 | 0.70 | 0.0009 | 0.0076 | 0.024 | 0.026 |
| 2478155 | | | | | | | | | | | | | | | | | |
| svm:2332285 | 0.71 | 0.0024 | 0.0002 | 0.0002 | 0.74 | 0.0001 | 0.67 | 0.82 | 0.79 | 0.71 | −0.19 | 0.0001 | 0.71 | 0.0009 | 0.0006 | 0.022 | 0.003 |
| 3463598 | | | | | | | | | | | | | | | | | |
| rf:2789391 | 0.72 | 0.0025 | 0.0511 | 0.0038 | 0.65 | 0.0093 | 0.39 | 0.91 | 0.81 | 0.60 | 0.56 | 0.0017 | 0.70 | 0.0033 | 0.0074 | 0.094 | 0.026 |
| 2651515 | | | | | | | | | | | | | | | | | |
| knn:3414753 | 0.71 | 0.0026 | 0.0255 | 0.0032 | 0.65 | 0.0093 | 0.48 | 0.82 | 0.73 | 0.61 | 0.55 | 0.0048 | 0.71 | 0.0032 | 0.0055 | 0.018 | 0.010 |
| 3066770 | | | | | | | | | | | | | | | | | |
| knn:2789391 | 0.71 | 0.0026 | 0.0053 | 0.0018 | 0.67 | 0.0046 | 0.48 | 0.85 | 0.76 | 0.62 | 0.51 | 0.0020 | 0.72 | 0.0012 | 0.0041 | 0.005 | 0.004 |
| 3066770 | | | | | | | | | | | | | | | | | |
| rpart:2478155 | 0.68 | 0.0028 | 0.0255 | 0.0021 | 0.68 | 0.0021 | 0.79 | 0.58 | 0.65 | 0.73 | 0.44 | 0.0020 | 0.71 | 0.0037 | 0.0034 | 0.000 | 0.001 |
| 3005357 | | | | | | | | | | | | | | | | | |
| rpart:2956442 | 0.68 | 0.0028 | 0.0255 | 0.0021 | 0.68 | 0.0021 | 0.79 | 0.58 | 0.65 | 0.73 | 0.44 | 0.0020 | 0.71 | 0.0037 | 0.0034 | 0.000 | 0.001 |
| 3005357 | | | | | | | | | | | | | | | | | |
| rpart:3139092 | 0.68 | 0.0028 | 0.0255 | 0.0021 | 0.68 | 0.0021 | 0.79 | 0.58 | 0.65 | 0.73 | 0.44 | 0.0020 | 0.71 | 0.0037 | 0.0034 | 0.000 | 0.001 |
| 3005357 | | | | | | | | | | | | | | | | | |
| nb:3414753 | 0.71 | 0.0028 | 0.0048 | 0.0012 | 0.67 | 0.0046 | 0.55 | 0.79 | 0.72 | 0.63 | 0.47 | 0.0054 | 0.74 | 0.0009 | 0.0031 | 0.034 | 0.013 |
| 3066770 | | | | | | | | | | | | | | | | | |
| nb:2789391 | 0.71 | 0.0029 | 0.0048 | 0.0031 | 0.67 | 0.0046 | 0.64 | 0.70 | 0.68 | 0.66 | 0.45 | 0.0055 | 0.73 | 0.0022 | 0.0054 | 0.004 | 0.003 |
| 3005357 | | | | | | | | | | | | | | | | | |
| knn:3139092 | 0.71 | 0.0030 | 0.0119 | 0.0029 | 0.67 | 0.0046 | 0.73 | 0.61 | 0.65 | 0.69 | 0.47 | 0.0090 | 0.73 | 0.0059 | 0.0048 | 0.012 | 0.004 |
| 3463598 | | | | | | | | | | | | | | | | | |
| nb:2332285 | 0.71 | 0.0032 | 0.0019 | 0.0035 | 0.68 | 0.0021 | 0.58 | 0.79 | 0.73 | 0.65 | 0.48 | 0.0036 | 0.71 | 0.0038 | 0.0064 | 0.034 | 0.014 |
| 2789391 | | | | | | | | | | | | | | | | | |
| nb:3326487 | 0.71 | 0.0032 | 0.0048 | 0.0076 | 0.58 | 0.1339 | 0.42 | 0.73 | 0.61 | 0.56 | 0.44 | 0.2432 | 0.71 | 0.0032 | 0.0146 | 0.094 | 0.068 |
| 3414753 | | | | | | | | | | | | | | | | | |
| svm:3139092 | 0.71 | 0.0032 | 0.0048 | 0.0033 | 0.67 | 0.0046 | 0.67 | 0.67 | 0.67 | 0.67 | −0.02 | 0.0076 | 0.72 | 0.0064 | 0.0056 | 0.005 | 0.003 |
| 3066770 | | | | | | | | | | | | | | | | | |
| knn:2332285 | 0.71 | 0.0032 | 0.0053 | 0.0021 | 0.65 | 0.0093 | 0.52 | 0.79 | 0.71 | 0.62 | 0.53 | 0.0115 | 0.70 | 0.0031 | 0.0038 | 0.021 | 0.006 |
| 2789391 | | | | | | | | | | | | | | | | | |
| rf:2789391 | 0.71 | 0.0032 | 0.0053 | 0.0062 | 0.61 | 0.0544 | 0.55 | 0.67 | 0.62 | 0.59 | 0.45 | 0.0582 | 0.71 | 0.0107 | 0.0088 | 0.018 | 0.004 |
| 2364325 | | | | | | | | | | | | | | | | | |
| nb:2461837 | 0.71 | 0.0033 | 0.0113 | 0.0027 | 0.65 | 0.0093 | 0.67 | 0.64 | 0.65 | 0.66 | 0.41 | 0.0120 | 0.72 | 0.0028 | 0.0059 | 0.080 | 0.013 |
| 2651515 | | | | | | | | | | | | | | | | | |
| nb:3463598 | 0.71 | 0.0033 | 0.0048 | 0.0013 | 0.70 | 0.0009 | 0.61 | 0.79 | 0.74 | 0.67 | 0.50 | 0.0011 | 0.72 | 0.0018 | 0.0029 | 0.011 | 0.006 |
| 2364325 | | | | | | | | | | | | | | | | | |
| rf:2461837 | 0.71 | 0.0035 | 0.0119 | 0.0018 | 0.68 | 0.0021 | 0.61 | 0.76 | 0.71 | 0.66 | 0.54 | 0.0031 | 0.70 | 0.0039 | 0.0031 | 0.008 | 0.002 |
| 2364325 | | | | | | | | | | | | | | | | | |
| rf:2789391 | 0.71 | 0.0035 | 0.0053 | 0.0024 | 0.70 | 0.0009 | 0.82 | 0.58 | 0.66 | 0.76 | 0.34 | 0.0012 | 0.73 | 0.0040 | 0.0043 | 0.002 | 0.001 |
| 3005357 | | | | | | | | | | | | | | | | | |
| nb:3326487 | 0.71 | 0.0036 | 0.0247 | 0.0036 | 0.59 | 0.0876 | 0.27 | 0.91 | 0.75 | 0.56 | 0.55 | 0.0518 | 0.72 | 0.0015 | 0.0081 | 0.023 | 0.029 |
| 2364325 | | | | | | | | | | | | | | | | | |
| rf:2458376 | 0.71 | 0.0037 | 0.0119 | 0.0027 | 0.64 | 0.0178 | 0.79 | 0.48 | 0.60 | 0.70 | 0.48 | 0.0533 | 0.70 | 0.0113 | 0.0051 | 0.091 | 0.010 |
| 2478155 | | | | | | | | | | | | | | | | | |
| rf:3463598 | 0.71 | 0.0037 | 0.0053 | 0.0034 | 0.70 | 0.0009 | 0.76 | 0.64 | 0.68 | 0.72 | 0.47 | 0.0027 | 0.69 | 0.0095 | 0.0054 | 0.125 | 0.015 |
| 2461837 | | | | | | | | | | | | | | | | | |
| nb:2458376 | 0.71 | 0.0038 | 0.0007 | 0.0013 | 0.67 | 0.0046 | 0.48 | 0.85 | 0.76 | 0.62 | 0.49 | 0.0038 | 0.71 | 0.0026 | 0.0038 | 0.081 | 0.017 |
| 2789391 | | | | | | | | | | | | | | | | | |
| rf:2458376 | 0.71 | 0.0038 | 0.0053 | 0.0010 | 0.70 | 0.0009 | 0.55 | 0.85 | 0.78 | 0.65 | 0.53 | 0.0013 | 0.70 | 0.0028 | 0.0024 | 0.024 | 0.003 |
| 2789391 | | | | | | | | | | | | | | | | | |
| rf:3463598 | 0.71 | 0.0038 | 0.0053 | 0.0022 | 0.65 | 0.0093 | 0.70 | 0.61 | 0.64 | 0.67 | 0.43 | 0.0229 | 0.71 | 0.0071 | 0.0038 | 0.119 | 0.014 |
| 2956442 | | | | | | | | | | | | | | | | | |
| knn:3139092 | 0.71 | 0.0038 | 0.0053 | 0.0031 | 0.67 | 0.0046 | 0.64 | 0.70 | 0.68 | 0.66 | 0.54 | 0.0056 | 0.72 | 0.0060 | 0.0048 | 0.002 | 0.002 |
| 2364325 | | | | | | | | | | | | | | | | | |
| knn:3005357 | 0.70 | 0.0039 | 0.0119 | 0.0047 | 0.58 | 0.1339 | 0.42 | 0.73 | 0.61 | 0.56 | 0.50 | 0.1856 | 0.74 | 0.0055 | 0.0078 | 0.023 | 0.006 |
| 2651515 | | | | | | | | | | | | | | | | | |
| rf:2478155 | 0.71 | 0.0039 | 0.0053 | 0.0013 | 0.67 | 0.0046 | 0.79 | 0.55 | 0.63 | 0.72 | 0.55 | 0.0091 | 0.70 | 0.0044 | 0.0030 | 0.012 | 0.002 |
| 3139092 | | | | | | | | | | | | | | | | | |
| nb:2332285 | 0.70 | 0.0039 | 0.0007 | 0.0019 | 0.70 | 0.0009 | 0.67 | 0.73 | 0.71 | 0.69 | 0.46 | 0.0035 | 0.73 | 0.0022 | 0.0039 | 0.023 | 0.012 |
| 2461837 | | | | | | | | | | | | | | | | | |
| rf:2332285 | 0.71 | 0.0040 | 0.0022 | 0.0095 | 0.61 | 0.0544 | 0.70 | 0.52 | 0.59 | 0.63 | 0.37 | 0.1214 | 0.67 | 0.0182 | 0.0122 | 0.126 | 0.027 |
| 2461837 | | | | | | | | | | | | | | | | | |
| rf:2332285 | 0.71 | 0.0040 | 0.0022 | 0.0032 | 0.70 | 0.0009 | 0.82 | 0.58 | 0.66 | 0.76 | 0.39 | 0.0019 | 0.73 | 0.0060 | 0.0054 | 0.011 | 0.003 |
| 3139092 | | | | | | | | | | | | | | | | | |
| rpart:2332285 | 0.70 | 0.0042 | 0.0255 | 0.0129 | 0.64 | 0.0178 | 0.58 | 0.70 | 0.66 | 0.62 | 0.44 | 0.0060 | 0.72 | 0.0043 | 0.0154 | 0.002 | 0.003 |
| 3005357 | | | | | | | | | | | | | | | | | |
| knn:2478155 | 0.70 | 0.0042 | 0.0119 | 0.0009 | 0.68 | 0.0021 | 0.91 | 0.45 | 0.63 | 0.83 | 0.45 | 0.0041 | 0.70 | 0.0055 | 0.0028 | 0.039 | 0.006 |
| 2461837 | | | | | | | | | | | | | | | | | |
| nb:3066770 | 0.70 | 0.0043 | 0.0113 | 0.0023 | 0.64 | 0.0178 | 0.30 | 0.97 | 0.91 | 0.58 | 0.54 | 0.0004 | 0.70 | 0.0018 | 0.0073 | 0.106 | 0.018 |
| 2651515 | | | | | | | | | | | | | | | | | |
| svm:3005357 | 0.70 | 0.0043 | 0.0505 | 0.0095 | 0.65 | 0.0093 | 0.55 | 0.76 | 0.69 | 0.63 | −0.36 | 0.0078 | 0.72 | 0.0056 | 0.0147 | 0.045 | 0.008 |
| 2651515 | | | | | | | | | | | | | | | | | |
| svm:3326487 | 0.70 | 0.0043 | 0.0113 | 0.0094 | 0.56 | 0.1945 | 0.39 | 0.73 | 0.59 | 0.55 | −0.21 | 0.3468 | 0.70 | 0.0053 | 0.0149 | 0.075 | 0.110 |
| 2332285 | | | | | | | | | | | | | | | | | |
| rf:3463598 | 0.70 | 0.0043 | 0.0255 | 0.0010 | 0.68 | 0.0021 | 0.45 | 0.91 | 0.83 | 0.63 | 0.61 | 0.0014 | 0.71 | 0.0028 | 0.0030 | 0.252 | 0.015 |
| 2512364 | | | | | | | | | | | | | | | | | |
| rpart:3414753 | 0.69 | 0.0045 | 0.0255 | 0.0018 | 0.68 | 0.0021 | 0.55 | 0.82 | 0.75 | 0.64 | 0.57 | 0.0019 | 0.67 | 0.0040 | 0.0032 | 0.055 | 0.015 |
| 2461837 | | | | | | | | | | | | | | | | | |
| nb:2461837 | 0.70 | 0.0045 | 0.0048 | 0.0022 | 0.71 | 0.0004 | 0.70 | 0.73 | 0.72 | 0.71 | 0.46 | 0.0009 | 0.67 | 0.0033 | 0.0038 | 0.021 | 0.005 |
| 2512364 | | | | | | | | | | | | | | | | | |
| nb:3326487 | 0.70 | 0.0045 | 0.0247 | 0.0043 | 0.56 | 0.1945 | 0.33 | 0.79 | 0.61 | 0.54 | 0.50 | 0.3390 | 0.69 | 0.0024 | 0.0081 | 0.047 | 0.028 |
| 2512364 | | | | | | | | | | | | | | | | | |
| nb:3414753 | 0.70 | 0.0045 | 0.0048 | 0.0020 | 0.70 | 0.0009 | 0.67 | 0.73 | 0.71 | 0.69 | 0.45 | 0.0022 | 0.72 | 0.0018 | 0.0040 | 0.030 | 0.010 |
| 2461837 | | | | | | | | | | | | | | | | | |
| knn:2478155 | 0.70 | 0.0045 | 0.0255 | 0.0028 | 0.61 | 0.0544 | 0.61 | 0.61 | 0.61 | 0.61 | 0.50 | 0.0687 | 0.69 | 0.0083 | 0.0055 | 0.016 | 0.002 |
| 3066770 | | | | | | | | | | | | | | | | | |
| knn:3139092 | 0.70 | 0.0045 | 0.0119 | 0.0039 | 0.65 | 0.0093 | 0.58 | 0.73 | 0.68 | 0.63 | 0.50 | 0.0137 | 0.72 | 0.0066 | 0.0060 | 0.004 | 0.004 |
| 3066770 | | | | | | | | | | | | | | | | | |
| knn:3414753 | 0.70 | 0.0046 | 0.0119 | 0.0015 | 0.68 | 0.0021 | 0.61 | 0.76 | 0.71 | 0.66 | 0.48 | 0.0022 | 0.76 | 0.0024 | 0.0030 | 0.010 | 0.003 |
| 2364325 | | | | | | | | | | | | | | | | | |
| nb:2789391 | 0.70 | 0.0047 | 0.0247 | 0.0033 | 0.67 | 0.0046 | 0.64 | 0.70 | 0.68 | 0.66 | 0.44 | 0.0045 | 0.73 | 0.0032 | 0.0059 | 0.009 | 0.006 |
| 2364325 | | | | | | | | | | | | | | | | | |
| nb:3414753 | 0.70 | 0.0047 | 0.0048 | 0.0013 | 0.70 | 0.0009 | 0.61 | 0.79 | 0.74 | 0.67 | 0.49 | 0.0026 | 0.72 | 0.0014 | 0.0032 | 0.050 | 0.014 |
| 3463598 | | | | | | | | | | | | | | | | | |
| knn:2478155 | 0.70 | 0.0048 | 0.0966 | 0.0046 | 0.64 | 0.0178 | 0.52 | 0.76 | 0.68 | 0.61 | 0.52 | 0.0248 | 0.70 | 0.0102 | 0.0068 | 0.034 | 0.005 |
| 2956442 | | | | | | | | | | | | | | | | | |
| knn:3139092 | 0.70 | 0.0048 | 0.0022 | 0.0013 | 0.71 | 0.0004 | 0.88 | 0.55 | 0.66 | 0.82 | 0.41 | 0.0005 | 0.73 | 0.0015 | 0.0030 | 0.000 | 0.002 |
| 3005357 | | | | | | | | | | | | | | | | | |
| svm:3414753 | 0.70 | 0.0049 | 0.0048 | 0.0018 | 0.71 | 0.0004 | 0.67 | 0.76 | 0.73 | 0.69 | −0.07 | 0.0010 | 0.72 | 0.0048 | 0.0031 | 0.061 | 0.006 |
| 3463598 | | | | | | | | | | | | | | | | | |
| knn:2461837 | 0.70 | 0.0049 | 0.0255 | 0.0043 | 0.68 | 0.0021 | 0.55 | 0.82 | 0.75 | 0.64 | 0.59 | 0.0006 | 0.69 | 0.0051 | 0.0068 | 0.005 | 0.004 |
| 2364325 | | | | | | | | | | | | | | | | | |
| rpart:3326487 | 0.68 | 0.0051 | 0.0966 | 0.0038 | 0.65 | 0.0093 | 0.76 | 0.55 | 0.63 | 0.69 | 0.41 | 0.0259 | 0.69 | 0.0044 | 0.0063 | 0.054 | 0.009 |
| 2458376 | | | | | | | | | | | | | | | | | |
| nb:2458376 | 0.70 | 0.0051 | 0.0002 | 0.0013 | 0.70 | 0.0009 | 0.61 | 0.79 | 0.74 | 0.67 | 0.45 | 0.0043 | 0.69 | 0.0023 | 0.0033 | 0.093 | 0.021 |
| 2461837 | | | | | | | | | | | | | | | | | |
| rf:3414753 | 0.70 | 0.0052 | 0.0255 | 0.0034 | 0.65 | 0.0093 | 0.55 | 0.76 | 0.69 | 0.63 | 0.53 | 0.0146 | 0.75 | 0.0055 | 0.0054 | 0.009 | 0.003 |
| 3005357 | | | | | | | | | | | | | | | | | |
| knn:2512364 | 0.70 | 0.0052 | 0.0511 | 0.0070 | 0.64 | 0.0178 | 0.42 | 0.85 | 0.74 | 0.60 | 0.70 | 0.0117 | 0.69 | 0.0149 | 0.0101 | 0.111 | 0.016 |
| 2364325 | | | | | | | | | | | | | | | | | |
| knn:2458376 | 0.70 | 0.0053 | 0.0119 | 0.0020 | 0.65 | 0.0093 | 0.55 | 0.76 | 0.69 | 0.63 | 0.53 | 0.0210 | 0.69 | 0.0058 | 0.0038 | 0.046 | 0.008 |
| 2956442 | | | | | | | | | | | | | | | | | |
| rf:2789391 | 0.70 | 0.0054 | 0.0119 | 0.0024 | 0.65 | 0.0093 | 0.52 | 0.79 | 0.71 | 0.62 | 0.50 | 0.0081 | 0.69 | 0.0039 | 0.0041 | 0.024 | 0.007 |
| 3463598 | | | | | | | | | | | | | | | | | |
| nb:2458376 | 0.70 | 0.0055 | 0.0019 | 0.0008 | 0.70 | 0.0009 | 0.42 | 0.97 | 0.93 | 0.63 | 0.54 | 0.0003 | 0.71 | 0.0018 | 0.0031 | 0.138 | 0.026 |
| 3463598 | | | | | | | | | | | | | | | | | |
| nb:2789391 | 0.70 | 0.0055 | 0.0019 | 0.0030 | 0.70 | 0.0009 | 0.70 | 0.70 | 0.70 | 0.70 | 0.45 | 0.0020 | 0.69 | 0.0034 | 0.0052 | 0.017 | 0.004 |
| 2512364 | | | | | | | | | | | | | | | | | |
| svm:2364325 | 0.70 | 0.0055 | 0.0048 | 0.0055 | 0.64 | 0.0178 | 0.48 | 0.79 | 0.70 | 0.60 | 0.04 | 0.0239 | 0.72 | 0.0090 | 0.0080 | 0.018 | 0.005 |
| 2956442 | | | | | | | | | | | | | | | | | |
| svm:2789391 | 0.70 | 0.0055 | 0.0048 | 0.0097 | 0.67 | 0.0046 | 0.58 | 0.76 | 0.70 | 0.64 | −0.31 | 0.0072 | 0.66 | 0.0165 | 0.0138 | 0.150 | 0.034 |
| 2512364 | | | | | | | | | | | | | | | | | |
| knn:2512364 | 0.69 | 0.0056 | 0.0119 | 0.0039 | 0.68 | 0.0021 | 0.61 | 0.76 | 0.71 | 0.66 | 0.50 | 0.0034 | 0.73 | 0.0041 | 0.0063 | 0.122 | 0.027 |
| 2651515 | | | | | | | | | | | | | | | | | |
| svm:3139092 | 0.70 | 0.0058 | 0.0247 | 0.0338 | 0.67 | 0.0046 | 0.82 | 0.52 | 0.63 | 0.74 | 0.01 | 0.0075 | 0.72 | 0.0377 | 0.0403 | 0.127 | 0.024 |
| 3463598 | | | | | | | | | | | | | | | | | |
| rf:2478155 | 0.70 | 0.0058 | 0.0255 | 0.0026 | 0.65 | 0.0093 | 0.79 | 0.52 | 0.62 | 0.71 | 0.38 | 0.0102 | 0.69 | 0.0073 | 0.0045 | 0.115 | 0.009 |
| 3066770 | | | | | | | | | | | | | | | | | |
| rf:2364325 | 0.70 | 0.0059 | 0.0053 | 0.0046 | 0.68 | 0.0021 | 0.58 | 0.79 | 0.73 | 0.65 | 0.54 | 0.0017 | 0.73 | 0.0056 | 0.0069 | 0.013 | 0.005 |
| 2956442 | | | | | | | | | | | | | | | | | |
| nb:3139092 | 0.70 | 0.0060 | 0.0007 | 0.0055 | 0.67 | 0.0046 | 0.61 | 0.73 | 0.69 | 0.65 | 0.46 | 0.0060 | 0.71 | 0.0069 | 0.0082 | 0.010 | 0.007 |
| 2461837 | | | | | | | | | | | | | | | | | |
| svm:2332285 | 0.70 | 0.0060 | 0.0247 | 0.0023 | 0.67 | 0.0046 | 0.58 | 0.76 | 0.70 | 0.64 | −0.30 | 0.0025 | 0.71 | 0.0018 | 0.0043 | 0.045 | 0.014 |
| 2651515 | | | | | | | | | | | | | | | | | |
| svm:2789391 | 0.70 | 0.0060 | 0.0247 | 0.0046 | 0.65 | 0.0093 | 0.64 | 0.67 | 0.66 | 0.65 | −0.23 | 0.0104 | 0.70 | 0.0039 | 0.0077 | 0.012 | 0.007 |
| 2364325 | | | | | | | | | | | | | | | | | |
| knn:2789391 | 0.70 | 0.0062 | 0.0966 | 0.0124 | 0.64 | 0.0178 | 0.45 | 0.82 | 0.71 | 0.60 | 0.51 | 0.0131 | 0.64 | 0.0165 | 0.0163 | 0.069 | 0.010 |
| 2651515 | | | | | | | | | | | | | | | | | |
| rf:2478155 | 0.70 | 0.0062 | 0.0119 | 0.0029 | 0.68 | 0.0021 | 0.79 | 0.58 | 0.65 | 0.73 | 0.44 | 0.0048 | 0.71 | 0.0078 | 0.0051 | 0.057 | 0.005 |
| 2956442 | | | | | | | | | | | | | | | | | |
| rf:2332285 | 0.70 | 0.0063 | 0.0053 | 0.0039 | 0.65 | 0.0093 | 0.48 | 0.82 | 0.73 | 0.61 | 0.54 | 0.0054 | 0.68 | 0.0062 | 0.0063 | 0.035 | 0.007 |
| 2789391 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 2332285 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 2364325 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 2478155 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 2789391 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 3005357 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 3066770 | | | | | | | | | | | | | | | | | |
| rpart:2458376 | 0.67 | 0.0064 | 0.0511 | 0.0054 | 0.67 | 0.0046 | 0.76 | 0.58 | 0.64 | 0.70 | 0.48 | 0.0151 | 0.66 | 0.0193 | 0.0072 | 0.042 | 0.004 |
| 3139092 | | | | | | | | | | | | | | | | | |
| nb:2332285 | 0.69 | 0.0065 | 0.0247 | 0.0094 | 0.67 | 0.0046 | 0.55 | 0.79 | 0.72 | 0.63 | 0.49 | 0.0107 | 0.72 | 0.0121 | 0.0130 | 0.054 | 0.019 |
| 2956442 | | | | | | | | | | | | | | | | | |
| rf:3139092 | 0.69 | 0.0067 | 0.0119 | 0.0081 | 0.65 | 0.0093 | 0.73 | 0.58 | 0.63 | 0.68 | 0.54 | 0.0173 | 0.72 | 0.0154 | 0.0113 | 0.049 | 0.007 |
| 3463598 | | | | | | | | | | | | | | | | | |
| knn:2458376 | 0.69 | 0.0068 | 0.0255 | 0.0066 | 0.62 | 0.0320 | 0.70 | 0.55 | 0.61 | 0.64 | 0.45 | 0.0693 | 0.68 | 0.0151 | 0.0090 | 0.075 | 0.014 |
| 2461837 | | | | | | | | | | | | | | | | | |
| nb:3139092 | 0.69 | 0.0068 | 0.0247 | 0.0066 | 0.68 | 0.0021 | 0.55 | 0.82 | 0.75 | 0.64 | 0.49 | 0.0022 | 0.69 | 0.0054 | 0.0104 | 0.008 | 0.005 |
| 2789391 | | | | | | | | | | | | | | | | | |
| nb:3326487 | 0.69 | 0.0068 | 0.0113 | 0.0155 | 0.56 | 0.1945 | 0.30 | 0.82 | 0.63 | 0.54 | 0.47 | 0.2595 | 0.69 | 0.0067 | 0.0239 | 0.107 | 0.144 |
| 3066770 | | | | | | | | | | | | | | | | | |
| svm:2478155 | 0.69 | 0.0068 | 0.0247 | 0.0045 | 0.65 | 0.0093 | 0.73 | 0.58 | 0.63 | 0.68 | 0.12 | 0.0253 | 0.63 | 0.0132 | 0.0069 | 0.077 | 0.013 |
| 3463598 | | | | | | | | | | | | | | | | | |
| svm:3326487 | 0.69 | 0.0068 | 0.0048 | 0.0145 | 0.55 | 0.2693 | 0.36 | 0.73 | 0.57 | 0.53 | 0.02 | 0.4287 | 0.71 | 0.0162 | 0.0187 | 0.067 | 0.063 |
| 3005357 | | | | | | | | | | | | | | | | | |
| knn:2461837 | 0.69 | 0.0069 | 0.0022 | 0.0052 | 0.73 | 0.0001 | 0.73 | 0.73 | 0.73 | 0.73 | 0.50 | 0.0002 | 0.67 | 0.0098 | 0.0075 | 0.055 | 0.005 |
| 2512364 | | | | | | | | | | | | | | | | | |
| knn:2512364 | 0.69 | 0.0070 | 0.0966 | 0.0065 | 0.62 | 0.0320 | 0.61 | 0.64 | 0.63 | 0.62 | 0.50 | 0.0440 | 0.70 | 0.0052 | 0.0092 | 0.009 | 0.004 |
| 3005357 | | | | | | | | | | | | | | | | | |
| nb:3414753 | 0.69 | 0.0071 | 0.0048 | 0.0034 | 0.67 | 0.0046 | 0.64 | 0.70 | 0.68 | 0.66 | 0.44 | 0.0083 | 0.70 | 0.0026 | 0.0065 | 0.032 | 0.009 |
| 2789391 | | | | | | | | | | | | | | | | | |
| nb:3139092 | 0.69 | 0.0073 | 0.0048 | 0.0039 | 0.68 | 0.0021 | 0.64 | 0.73 | 0.70 | 0.67 | 0.47 | 0.0049 | 0.72 | 0.0039 | 0.0066 | 0.007 | 0.008 |
| 3463598 | | | | | | | | | | | | | | | | | |
| rf:2364325 | 0.69 | 0.0073 | 0.0119 | 0.0014 | 0.68 | 0.0021 | 0.76 | 0.61 | 0.66 | 0.71 | 0.47 | 0.0025 | 0.73 | 0.0025 | 0.0027 | 0.000 | 0.001 |
| 3005357 | | | | | | | | | | | | | | | | | |
| rpart:2458376 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| rpart:2461837 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2364325 | | | | | | | | | | | | | | | | | |
| rpart:2461837 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2956442 | | | | | | | | | | | | | | | | | |
| rpart:2461837 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 3005357 | | | | | | | | | | | | | | | | | |
| rpart:2478155 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| rpart:2789391 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| rpart:3139092 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| rpart:3326487 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| rpart:3463598 | 0.67 | 0.0074 | 0.0511 | 0.0062 | 0.67 | 0.0046 | 0.67 | 0.67 | 0.67 | 0.67 | 0.47 | 0.0097 | 0.65 | 0.0125 | 0.0079 | 0.038 | 0.012 |
| 2461837 | | | | | | | | | | | | | | | | | |
| knn:2332285 | 0.69 | 0.0074 | 0.0255 | 0.0093 | 0.68 | 0.0021 | 0.64 | 0.73 | 0.70 | 0.67 | 0.50 | 0.0034 | 0.69 | 0.0131 | 0.0131 | 0.067 | 0.007 |
| 3005357 | | | | | | | | | | | | | | | | | |
| rf:3005357 | 0.69 | 0.0075 | 0.0022 | 0.0075 | 0.65 | 0.0093 | 0.61 | 0.70 | 0.67 | 0.64 | 0.45 | 0.0103 | 0.73 | 0.0088 | 0.0101 | 0.064 | 0.045 |
| 2651515 | | | | | | | | | | | | | | | | | |
| svm:2458376 | 0.69 | 0.0076 | 0.0247 | 0.0066 | 0.62 | 0.0320 | 0.58 | 0.67 | 0.63 | 0.61 | 0.06 | 0.0842 | 0.67 | 0.0136 | 0.0096 | 0.037 | 0.008 |
| 2478155 | | | | | | | | | | | | | | | | | |
| knn:2478155 | 0.69 | 0.0077 | 0.0255 | 0.0070 | 0.65 | 0.0093 | 0.79 | 0.52 | 0.62 | 0.71 | 0.44 | 0.0207 | 0.66 | 0.0218 | 0.0105 | 0.371 | 0.031 |
| 3463598 | | | | | | | | | | | | | | | | | |
| knn:2458376 | 0.69 | 0.0077 | 0.0511 | 0.0081 | 0.67 | 0.0046 | 0.64 | 0.70 | 0.68 | 0.66 | 0.50 | 0.0174 | 0.70 | 0.0149 | 0.0112 | 0.209 | 0.047 |
| 2332285 | | | | | | | | | | | | | | | | | |
| rf:2458376 | 0.69 | 0.0078 | 0.0053 | 0.0044 | 0.65 | 0.0093 | 0.39 | 0.91 | 0.81 | 0.60 | 0.55 | 0.0017 | 0.70 | 0.0039 | 0.0083 | 0.127 | 0.031 |
| 2651515 | | | | | | | | | | | | | | | | | |
| rf:2789391 | 0.69 | 0.0079 | 0.0053 | 0.0031 | 0.70 | 0.0009 | 0.67 | 0.73 | 0.71 | 0.69 | 0.45 | 0.0019 | 0.68 | 0.0056 | 0.0049 | 0.027 | 0.004 |
| 2512364 | | | | | | | | | | | | | | | | | |
| rf:3326487 | 0.69 | 0.0079 | 0.0255 | 0.0042 | 0.58 | 0.1339 | 0.27 | 0.88 | 0.69 | 0.55 | 0.60 | 0.1299 | 0.73 | 0.0028 | 0.0079 | 0.030 | 0.021 |
| 2364325 | | | | | | | | | | | | | | | | | |
| svm:3414753 | 0.69 | 0.0080 | 0.0247 | 0.0078 | 0.65 | 0.0093 | 0.55 | 0.76 | 0.69 | 0.63 | −0.17 | 0.0108 | 0.72 | 0.0085 | 0.0115 | 0.047 | 0.009 |
| 2956442 | | | | | | | | | | | | | | | | | |
| knn:2332285 | 0.69 | 0.0080 | 0.0053 | 0.0027 | 0.70 | 0.0009 | 0.67 | 0.73 | 0.71 | 0.69 | 0.50 | 0.0021 | 0.72 | 0.0033 | 0.0045 | 0.027 | 0.007 |
| 2364325 | | | | | | | | | | | | | | | | | |
| knn:2332285 | 0.69 | 0.0082 | 0.0003 | 0.0021 | 0.73 | 0.0001 | 0.64 | 0.82 | 0.78 | 0.69 | 0.55 | 0.0002 | 0.72 | 0.0051 | 0.0039 | 0.135 | 0.015 |
| 2512364 | | | | | | | | | | | | | | | | | |
| rf:2956442 | 0.69 | 0.0082 | 0.0053 | 0.0098 | 0.64 | 0.0178 | 0.64 | 0.64 | 0.64 | 0.64 | 0.46 | 0.0233 | 0.71 | 0.0135 | 0.0124 | 0.004 | 0.002 |
| 3005357 | | | | | | | | | | | | | | | | | |
| svm:3326487 | 0.69 | 0.0083 | 0.0505 | 0.0184 | 0.58 | 0.1339 | 0.39 | 0.76 | 0.62 | 0.56 | −0.18 | 0.2183 | 0.70 | 0.0141 | 0.0241 | 0.121 | 0.127 |
| 2364325 | | | | | | | | | | | | | | | | | |
| knn:3414753 | 0.69 | 0.0084 | 0.0511 | 0.0092 | 0.61 | 0.0544 | 0.67 | 0.55 | 0.59 | 0.62 | 0.45 | 0.1123 | 0.70 | 0.0156 | 0.0124 | 0.198 | 0.020 |
| 2478155 | | | | | | | | | | | | | | | | | |
| rpart:3066770 | 0.67 | 0.0084 | 0.0255 | 0.0067 | 0.64 | 0.0178 | 0.67 | 0.61 | 0.63 | 0.65 | 0.56 | 0.0238 | 0.69 | 0.0093 | 0.0087 | 0.001 | 0.002 |
| 3005357 | | | | | | | | | | | | | | | | | |
| rf:2458376 | 0.69 | 0.0086 | 0.0511 | 0.0054 | 0.67 | 0.0046 | 0.64 | 0.70 | 0.68 | 0.66 | 0.50 | 0.0122 | 0.69 | 0.0154 | 0.0077 | 0.045 | 0.005 |
| 2956442 | | | | | | | | | | | | | | | | | |
| nb:2364325 | 0.69 | 0.0086 | 0.0048 | 0.0084 | 0.67 | 0.0046 | 0.55 | 0.79 | 0.72 | 0.63 | 0.50 | 0.0051 | 0.71 | 0.0106 | 0.0115 | 0.020 | 0.007 |
| 2956442 | | | | | | | | | | | | | | | | | |
| svm:3414753 | 0.69 | 0.0086 | 0.0113 | 0.0062 | 0.68 | 0.0021 | 0.70 | 0.67 | 0.68 | 0.69 | −0.26 | 0.0048 | 0.69 | 0.0044 | 0.0101 | 0.034 | 0.011 |
| 2789391 | | | | | | | | | | | | | | | | | |
| rf:2461837 | 0.69 | 0.0089 | 0.0053 | 0.0040 | 0.65 | 0.0093 | 0.64 | 0.67 | 0.66 | 0.65 | 0.46 | 0.0114 | 0.71 | 0.0076 | 0.0058 | 0.006 | 0.002 |
| 3005357 | | | | | | | | | | | | | | | | | |
| rpart:2332285 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:2364325 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:2478155 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:2512364 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:2789391 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:2956442 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:3005357 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:3066770 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:3326487 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:3414753 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rpart:3463598 | 0.64 | 0.0089 | 0.1717 | 0.0077 | 0.64 | 0.0178 | 0.36 | 0.91 | 0.80 | 0.59 | 0.56 | 0.0018 | 0.67 | 0.0029 | 0.0135 | 0.118 | 0.048 |
| 2651515 | | | | | | | | | | | | | | | | | |
| rf:2478155 | 0.69 | 0.0091 | 0.0053 | 0.0159 | 0.59 | 0.0876 | 0.70 | 0.48 | 0.58 | 0.62 | 0.43 | 0.2117 | 0.66 | 0.0253 | 0.0193 | 0.379 | 0.044 |
| 2512364 | | | | | | | | | | | | | | | | | |
| knn:3414753 | 0.69 | 0.0091 | 0.0053 | 0.0013 | 0.70 | 0.0009 | 0.61 | 0.79 | 0.74 | 0.67 | 0.50 | 0.0026 | 0.73 | 0.0056 | 0.0027 | 0.128 | 0.009 |
| 3463598 | | | | | | | | | | | | | | | | | |
| knn:2332285 | 0.69 | 0.0091 | 0.0009 | 0.0013 | 0.71 | 0.0004 | 0.67 | 0.76 | 0.73 | 0.69 | 0.50 | 0.0005 | 0.75 | 0.0030 | 0.0024 | 0.007 | 0.003 |
| 3139092 | | | | | | | | | | | | | | | | | |
| rf:2956442 | 0.69 | 0.0092 | 0.0255 | 0.0047 | 0.62 | 0.0320 | 0.30 | 0.94 | 0.83 | 0.57 | 0.58 | 0.0085 | 0.70 | 0.0036 | 0.0091 | 0.127 | 0.040 |
| 2651515 | | | | | | | | | | | | | | | | | |
| rf:2478155 | 0.69 | 0.0092 | 0.0255 | 0.0038 | 0.67 | 0.0046 | 0.61 | 0.73 | 0.69 | 0.65 | 0.64 | 0.0098 | 0.64 | 0.0114 | 0.0062 | 0.075 | 0.009 |
| 2461837 | | | | | | | | | | | | | | | | | |
| rf:2458376 | 0.69 | 0.0092 | 0.0255 | 0.0125 | 0.61 | 0.0544 | 0.48 | 0.73 | 0.64 | 0.59 | 0.55 | 0.0825 | 0.67 | 0.0291 | 0.0166 | 0.297 | 0.021 |
| 3066770 | | | | | | | | | | | | | | | | | |
| svm:3463598 | 0.69 | 0.0093 | 0.0048 | 0.0025 | 0.71 | 0.0004 | 0.58 | 0.85 | 0.79 | 0.67 | −0.29 | 0.0004 | 0.69 | 0.0013 | 0.0057 | 0.027 | 0.026 |
| 3066770 | | | | | | | | | | | | | | | | | |
| knn:2364325 | 0.68 | 0.0095 | 0.1717 | 0.0136 | 0.64 | 0.0178 | 0.33 | 0.94 | 0.85 | 0.58 | 0.61 | 0.0004 | 0.70 | 0.0064 | 0.0203 | 0.023 | 0.035 |
| 2651515 | | | | | | | | | | | | | | | | | |
| nb:2461837 | 0.68 | 0.0097 | 0.0048 | 0.0067 | 0.64 | 0.0178 | 0.52 | 0.76 | 0.68 | 0.61 | 0.48 | 0.0092 | 0.68 | 0.0057 | 0.0097 | 0.045 | 0.026 |
| 3066770 | | | | | | | | | | | | | | | | | |
| rf:3414753 | 0.69 | 0.0098 | 0.0022 | 0.0018 | 0.68 | 0.0021 | 0.64 | 0.73 | 0.70 | 0.67 | 0.52 | 0.0043 | 0.75 | 0.0037 | 0.0032 | 0.018 | 0.004 |
| 2364325 | | | | | | | | | | | | | | | | | |
| knn:2364325 | 0.68 | 0.0100 | 0.0255 | 0.0072 | 0.65 | 0.0093 | 0.55 | 0.76 | 0.69 | 0.63 | 0.54 | 0.0128 | 0.73 | 0.0124 | 0.0098 | 0.010 | 0.004 |
| 2956442 | | | | | | | | | | | | | | | | | |
| knn:3139092 | 0.68 | 0.0102 | 0.0511 | 0.0126 | 0.64 | 0.0178 | 0.79 | 0.48 | 0.60 | 0.70 | 0.44 | 0.0243 | 0.73 | 0.0142 | 0.0169 | 0.002 | 0.002 |
| 2956442 | | | | | | | | | | | | | | | | | |
| knn:3139092 | 0.68 | 0.0104 | 0.0255 | 0.0045 | 0.68 | 0.0021 | 0.61 | 0.76 | 0.71 | 0.66 | 0.48 | 0.0020 | 0.71 | 0.0057 | 0.0066 | 0.009 | 0.004 |
| 2789391 | | | | | | | | | | | | | | | | | |
| knn:2789391 | 0.68 | 0.0104 | 0.0511 | 0.0081 | 0.65 | 0.0093 | 0.55 | 0.76 | 0.69 | 0.63 | 0.50 | 0.0097 | 0.69 | 0.0091 | 0.0109 | 0.037 | 0.009 |
| 2461837 | | | | | | | | | | | | | | | | | |
| nb:3326487 | 0.68 | 0.0109 | 0.0247 | 0.0042 | 0.68 | 0.0021 | 0.42 | 0.94 | 0.88 | 0.62 | 0.49 | 0.0009 | 0.65 | 0.0033 | 0.0099 | 0.169 | 0.098 |
| 2458376 | | | | | | | | | | | | | | | | | |
| nb:2789391 | 0.68 | 0.0113 | 0.0113 | 0.0062 | 0.64 | 0.0178 | 0.52 | 0.76 | 0.68 | 0.61 | 0.44 | 0.0136 | 0.69 | 0.0050 | 0.0118 | 0.084 | 0.012 |
| 2651515 | | | | | | | | | | | | | | | | | |
| nb:3326487 | 0.68 | 0.0113 | 0.0247 | 0.0137 | 0.59 | 0.0876 | 0.45 | 0.73 | 0.63 | 0.57 | 0.43 | 0.1452 | 0.71 | 0.0060 | 0.0202 | 0.030 | 0.041 |
| 3139092 | | | | | | | | | | | | | | | | | |
| svm:3326487 | 0.68 | 0.0113 | 0.0247 | 0.0061 | 0.61 | 0.0544 | 0.48 | 0.73 | 0.64 | 0.59 | −0.08 | 0.1575 | 0.66 | 0.0134 | 0.0092 | 0.224 | 0.081 |
| 2458376 | | | | | | | | | | | | | | | | | |
| rpart:3326487 | 0.66 | 0.0115 | 0.1717 | 0.0083 | 0.64 | 0.0178 | 0.70 | 0.58 | 0.62 | 0.66 | 0.41 | 0.0263 | 0.69 | 0.0038 | 0.0121 | 0.021 | 0.007 |
| 3139092 | | | | | | | | | | | | | | | | | |
| rf:2789391 | 0.68 | 0.0115 | 0.0119 | 0.0098 | 0.65 | 0.0093 | 0.58 | 0.73 | 0.68 | 0.63 | 0.51 | 0.0116 | 0.65 | 0.0105 | 0.0128 | 0.048 | 0.019 |
| 3066770 | | | | | | | | | | | | | | | | | |
| nb:3414753 | 0.68 | 0.0117 | 0.0247 | 0.0100 | 0.65 | 0.0093 | 0.64 | 0.67 | 0.66 | 0.65 | 0.45 | 0.0240 | 0.72 | 0.0114 | 0.0135 | 0.057 | 0.012 |
| 2956442 | | | | | | | | | | | | | | | | | |
| svm:2789391 | 0.68 | 0.0117 | 0.0113 | 0.0076 | 0.68 | 0.0021 | 0.58 | 0.79 | 0.73 | 0.65 | −0.07 | 0.0025 | 0.68 | 0.0089 | 0.0108 | 0.045 | 0.013 |
| 3463598 | | | | | | | | | | | | | | | | | |
| rf:2789391 | 0.68 | 0.0117 | 0.0119 | 0.0078 | 0.65 | 0.0093 | 0.64 | 0.67 | 0.66 | 0.65 | 0.47 | 0.0240 | 0.65 | 0.0151 | 0.0102 | 0.032 | 0.008 |
| 2461837 | | | | | | | | | | | | | | | | | |
| svm:2461837 | 0.68 | 0.0122 | 0.0019 | 0.0078 | 0.71 | 0.0004 | 0.70 | 0.73 | 0.72 | 0.71 | 0.06 | 0.0007 | 0.67 | 0.0136 | 0.0104 | 0.017 | 0.005 |
| 3005357 | | | | | | | | | | | | | | | | | |
| nb:3463598 | 0.68 | 0.0126 | 0.0505 | 0.0109 | 0.65 | 0.0093 | 0.55 | 0.76 | 0.69 | 0.63 | 0.50 | 0.0103 | 0.67 | 0.0120 | 0.0140 | 0.074 | 0.030 |
| 2461837 | | | | | | | | | | | | | | | | | |
| svm:3463598 | 0.68 | 0.0126 | 0.0048 | 0.0022 | 0.70 | 0.0009 | 0.64 | 0.76 | 0.72 | 0.68 | 0.12 | 0.0018 | 0.65 | 0.0115 | 0.0049 | 0.553 | 0.051 |
| 2461837 | | | | | | | | | | | | | | | | | |
| knn:3463598 | 0.68 | 0.0128 | 0.0511 | 0.0033 | 0.67 | 0.0046 | 0.55 | 0.79 | 0.72 | 0.63 | 0.51 | 0.0079 | 0.70 | 0.0063 | 0.0053 | 0.024 | 0.005 |
| 2956442 | | | | | | | | | | | | | | | | | |
| rf:3414753 | 0.68 | 0.0133 | 0.0053 | 0.0029 | 0.67 | 0.0046 | 0.67 | 0.67 | 0.67 | 0.67 | 0.42 | 0.0109 | 0.72 | 0.0057 | 0.0046 | 0.082 | 0.012 |
| 3463598 | | | | | | | | | | | | | | | | | |
| rf:3139092 | 0.68 | 0.0133 | 0.0053 | 0.0139 | 0.64 | 0.0178 | 0.55 | 0.73 | 0.67 | 0.62 | 0.54 | 0.0260 | 0.67 | 0.0182 | 0.0173 | 0.017 | 0.003 |
| 2789391 | | | | | | | | | | | | | | | | | |
| nb:3463598 | 0.68 | 0.0136 | 0.0048 | 0.0041 | 0.70 | 0.0009 | 0.55 | 0.85 | 0.78 | 0.65 | 0.50 | 0.0006 | 0.68 | 0.0035 | 0.0070 | 0.053 | 0.028 |
| 3066770 | | | | | | | | | | | | | | | | | |
| nb:2478155 | 0.67 | 0.0141 | 0.0965 | 0.0068 | 0.64 | 0.0178 | 0.48 | 0.79 | 0.70 | 0.60 | 0.51 | 0.0223 | 0.69 | 0.0065 | 0.0099 | 0.013 | 0.006 |
| 2956442 | | | | | | | | | | | | | | | | | |
| nb:2512364 | 0.67 | 0.0141 | 0.0247 | 0.0085 | 0.67 | 0.0046 | 0.58 | 0.76 | 0.70 | 0.64 | 0.51 | 0.0076 | 0.70 | 0.0113 | 0.0113 | 0.033 | 0.007 |
| 2956442 | | | | | | | | | | | | | | | | | |
| rpart:3463598 | 0.67 | 0.0143 | 0.0255 | 0.0220 | 0.59 | 0.0876 | 0.67 | 0.52 | 0.58 | 0.61 | 0.49 | 0.2796 | 0.67 | 0.0556 | 0.0251 | 0.452 | 0.164 |
| 2956442 | | | | | | | | | | | | | | | | | |
| rpart:2332285 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 3139092 | | | | | | | | | | | | | | | | | |
| rpart:2478155 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 3139092 | | | | | | | | | | | | | | | | | |
| rpart:3139092 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 2364325 | | | | | | | | | | | | | | | | | |
| rpart:3139092 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 2789391 | | | | | | | | | | | | | | | | | |
| rpart:3139092 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 2956442 | | | | | | | | | | | | | | | | | |
| rpart:3139092 | 0.65 | 0.0145 | 0.0966 | 0.0130 | 0.65 | 0.0093 | 0.70 | 0.61 | 0.64 | 0.67 | 0.49 | 0.0134 | 0.67 | 0.0165 | 0.0151 | 0.003 | 0.002 |
| 3066770 | | | | | | | | | | | | | | | | | |
| nb:2789391 | 0.67 | 0.0147 | 0.0247 | 0.0089 | 0.67 | 0.0046 | 0.55 | 0.79 | 0.72 | 0.63 | 0.48 | 0.0035 | 0.67 | 0.0074 | 0.0129 | 0.038 | 0.015 |
| 3463598 | | | | | | | | | | | | | | | | | |
| nb:2956442 | 0.67 | 0.0147 | 0.0048 | 0.0150 | 0.62 | 0.0320 | 0.55 | 0.70 | 0.64 | 0.61 | 0.44 | 0.0460 | 0.70 | 0.0165 | 0.0212 | 0.140 | 0.016 |
| 2651515 | | | | | | | | | | | | | | | | | |
| nb:2956442 | 0.67 | 0.0147 | 0.0505 | 0.0108 | 0.65 | 0.0093 | 0.58 | 0.73 | 0.68 | 0.63 | 0.48 | 0.0139 | 0.73 | 0.0115 | 0.0139 | 0.010 | 0.005 |
| 3005357 | | | | | | | | | | | | | | | | | |
| nb:3139092 | 0.67 | 0.0147 | 0.0505 | 0.0203 | 0.64 | 0.0178 | 0.64 | 0.64 | 0.64 | 0.64 | 0.46 | 0.0385 | 0.71 | 0.0242 | 0.0242 | 0.014 | 0.006 |
| 2956442 | | | | | | | | | | | | | | | | | |
| rf:3139092 | 0.67 | 0.0150 | 0.0119 | 0.0019 | 0.67 | 0.0046 | 0.67 | 0.67 | 0.67 | 0.67 | 0.62 | 0.0043 | 0.73 | 0.0035 | 0.0036 | 0.001 | 0.001 |
| 3005357 | | | | | | | | | | | | | | | | | |
| nb:3139092 | 0.67 | 0.0152 | 0.0113 | 0.0027 | 0.67 | 0.0046 | 0.39 | 0.94 | 0.87 | 0.61 | 0.52 | 0.0001 | 0.70 | 0.0014 | 0.0063 | 0.029 | 0.006 |
| 2651515 | | | | | | | | | | | | | | | | | |
| svm:2789391 | 0.67 | 0.0152 | 0.0113 | 0.0093 | 0.67 | 0.0046 | 0.58 | 0.76 | 0.70 | 0.64 | −0.32 | 0.0045 | 0.66 | 0.0079 | 0.0146 | 0.069 | 0.012 |
| 2651515 | | | | | | | | | | | | | | | | | |
| svm:2789391 | 0.67 | 0.0152 | 0.0048 | 0.0074 | 0.70 | 0.0009 | 0.61 | 0.79 | 0.74 | 0.67 | −0.15 | 0.0006 | 0.67 | 0.0076 | 0.0103 | 0.038 | 0.015 |
| 3066770 | | | | | | | | | | | | | | | | | |
| knn:3326487 | 0.67 | 0.0153 | 0.0255 | 0.0227 | 0.58 | 0.1339 | 0.33 | 0.82 | 0.65 | 0.55 | 0.54 | 0.1316 | 0.66 | 0.0201 | 0.0301 | 0.208 | 0.055 |
| 2512364 | | | | | | | | | | | | | | | | | |
| knn:3414753 | 0.67 | 0.0157 | 0.0966 | 0.0229 | 0.64 | 0.0178 | 0.73 | 0.55 | 0.62 | 0.67 | 0.46 | 0.0336 | 0.70 | 0.0352 | 0.0270 | 0.027 | 0.011 |
| 3139092 | | | | | | | | | | | | | | | | | |
| knn:3463598 | 0.67 | 0.0163 | 0.4478 | 0.0090 | 0.61 | 0.0544 | 0.33 | 0.88 | 0.73 | 0.57 | 0.70 | 0.0478 | 0.69 | 0.0193 | 0.0131 | 0.066 | 0.017 |
| 2651515 | | | | | | | | | | | | | | | | | |
| svm:3139092 | 0.67 | 0.0164 | 0.0247 | 0.0193 | 0.65 | 0.0093 | 0.73 | 0.58 | 0.63 | 0.68 | −0.25 | 0.0220 | 0.67 | 0.0344 | 0.0228 | 0.034 | 0.011 |
| 2461837 | | | | | | | | | | | | | | | | | |
| svm:3463598 | 0.67 | 0.0170 | 0.0019 | 0.0028 | 0.71 | 0.0004 | 0.58 | 0.85 | 0.79 | 0.67 | 0.01 | 0.0004 | 0.68 | 0.0058 | 0.0049 | 0.086 | 0.009 |
| 2364325 | | | | | | | | | | | | | | | | | |
| knn:2478155 | 0.67 | 0.0173 | 0.0966 | 0.0301 | 0.64 | 0.0178 | 0.45 | 0.82 | 0.71 | 0.60 | 0.54 | 0.0097 | 0.65 | 0.0293 | 0.0344 | 0.053 | 0.010 |
| 2789391 | | | | | | | | | | | | | | | | | |
| rf:3414753 | 0.67 | 0.0173 | 0.0119 | 0.0101 | 0.68 | 0.0021 | 0.64 | 0.73 | 0.70 | 0.67 | 0.50 | 0.0043 | 0.70 | 0.0172 | 0.0128 | 0.045 | 0.011 |
| 2789391 | | | | | | | | | | | | | | | | | |
| svm:2332285 | 0.67 | 0.0176 | 0.0965 | 0.0183 | 0.64 | 0.0178 | 0.45 | 0.82 | 0.71 | 0.60 | −0.01 | 0.0145 | 0.66 | 0.0141 | 0.0226 | 0.045 | 0.017 |
| 2789391 | | | | | | | | | | | | | | | | | |
| knn:3463598 | 0.67 | 0.0184 | 0.0966 | 0.0144 | 0.61 | 0.0544 | 0.39 | 0.82 | 0.68 | 0.57 | 0.58 | 0.0632 | 0.67 | 0.0233 | 0.0185 | 0.189 | 0.012 |
| 3005357 | | | | | | | | | | | | | | | | | |
| rpart:2332285 | 0.66 | 0.0186 | 0.0511 | 0.0368 | 0.67 | 0.0046 | 0.61 | 0.73 | 0.69 | 0.65 | 0.59 | 0.0108 | 0.62 | 0.0559 | 0.0394 | 0.094 | 0.031 |
| 2461837 | | | | | | | | | | | | | | | | | |
| nb:2458376 | 0.67 | 0.0189 | 0.0048 | 0.0052 | 0.67 | 0.0046 | 0.55 | 0.79 | 0.72 | 0.63 | 0.46 | 0.0096 | 0.68 | 0.0101 | 0.0086 | 0.130 | 0.024 |
| 2956442 | | | | | | | | | | | | | | | | | |
| nb:2789391 | 0.67 | 0.0189 | 0.0048 | 0.0090 | 0.67 | 0.0046 | 0.61 | 0.73 | 0.69 | 0.65 | 0.44 | 0.0053 | 0.68 | 0.0061 | 0.0131 | 0.035 | 0.021 |
| 3066770 | | | | | | | | | | | | | | | | | |
| svm:2478155 | 0.67 | 0.0189 | 0.0505 | 0.0079 | 0.61 | 0.0544 | 0.55 | 0.67 | 0.62 | 0.59 | −0.13 | 0.0894 | 0.68 | 0.0048 | 0.0120 | 0.017 | 0.009 |
| 2789391 | | | | | | | | | | | | | | | | | |
| rf:2364325 | 0.67 | 0.0192 | 0.0966 | 0.0067 | 0.64 | 0.0178 | 0.36 | 0.91 | 0.80 | 0.59 | 0.73 | 0.0018 | 0.68 | 0.0036 | 0.0113 | 0.163 | 0.064 |
| 2651515 | | | | | | | | | | | | | | | | | |
| svm:3326487 | 0.67 | 0.0196 | 0.0113 | 0.0285 | 0.56 | 0.1945 | 0.36 | 0.76 | 0.60 | 0.54 | −0.35 | 0.3833 | 0.66 | 0.0144 | 0.0364 | 0.140 | 0.252 |
| 3066770 | | | | | | | | | | | | | | | | | |
| svm:3463598 | 0.67 | 0.0196 | 0.0019 | 0.0040 | 0.71 | 0.0004 | 0.61 | 0.82 | 0.77 | 0.68 | 0.02 | 0.0009 | 0.66 | 0.0088 | 0.0064 | 0.193 | 0.021 |
| 2512364 | | | | | | | | | | | | | | | | | |
| rf:3414753 | 0.67 | 0.0196 | 0.0255 | 0.0047 | 0.64 | 0.0178 | 0.64 | 0.64 | 0.64 | 0.64 | 0.35 | 0.0288 | 0.71 | 0.0042 | 0.0075 | 0.134 | 0.035 |
| 2651515 | | | | | | | | | | | | | | | | | |
| knn:2332285 | 0.66 | 0.0208 | 0.0053 | 0.0166 | 0.65 | 0.0093 | 0.52 | 0.79 | 0.71 | 0.62 | 0.53 | 0.0164 | 0.68 | 0.0250 | 0.0197 | 0.072 | 0.016 |
| 2956442 | | | | | | | | | | | | | | | | | |
| rf:3414753 | 0.67 | 0.0210 | 0.0511 | 0.0122 | 0.65 | 0.0093 | 0.52 | 0.79 | 0.71 | 0.62 | 0.58 | 0.0112 | 0.70 | 0.0175 | 0.0150 | 0.140 | 0.028 |
| 2332285 | | | | | | | | | | | | | | | | | |
| svm:2512364 | 0.66 | 0.0210 | 0.0505 | 0.0122 | 0.67 | 0.0046 | 0.58 | 0.76 | 0.70 | 0.64 | 0.04 | 0.0076 | 0.67 | 0.0155 | 0.0153 | 0.036 | 0.008 |
| 2956442 | | | | | | | | | | | | | | | | | |
| knn:3326487 | 0.66 | 0.0211 | 0.0255 | 0.0347 | 0.58 | 0.1339 | 0.24 | 0.91 | 0.73 | 0.55 | 0.61 | 0.0682 | 0.67 | 0.0278 | 0.0406 | 0.195 | 0.111 |
| 2364325 | | | | | | | | | | | | | | | | | |
| rf:3414753 | 0.67 | 0.0213 | 0.0255 | 0.0161 | 0.64 | 0.0178 | 0.55 | 0.73 | 0.67 | 0.62 | 0.50 | 0.0296 | 0.71 | 0.0224 | 0.0193 | 0.065 | 0.015 |
| 2956442 | | | | | | | | | | | | | | | | | |
| rf:3326487 | 0.66 | 0.0217 | 0.0966 | 0.0086 | 0.64 | 0.0178 | 0.33 | 0.94 | 0.85 | 0.58 | 0.60 | 0.0033 | 0.67 | 0.0091 | 0.0134 | 0.136 | 0.051 |
| 2458376 | | | | | | | | | | | | | | | | | |
| knn:2461837 | 0.66 | 0.0224 | 0.1717 | 0.0361 | 0.55 | 0.2693 | 0.30 | 0.79 | 0.59 | 0.53 | 0.64 | 0.4552 | 0.67 | 0.0563 | 0.0414 | 0.273 | 0.108 |
| 3066770 | | | | | | | | | | | | | | | | | |
| svm:3463598 | 0.66 | 0.0225 | 0.0247 | 0.0099 | 0.64 | 0.0178 | 0.48 | 0.79 | 0.70 | 0.60 | 0.02 | 0.0216 | 0.68 | 0.0162 | 0.0131 | 0.060 | 0.011 |
| 2956442 | | | | | | | | | | | | | | | | | |
| rpart:3139092 | 0.66 | 0.0226 | 0.1717 | 0.0219 | 0.62 | 0.0320 | 0.64 | 0.61 | 0.62 | 0.63 | 0.45 | 0.0407 | 0.70 | 0.0147 | 0.0255 | 0.161 | 0.032 |
| 2651515 | | | | | | | | | | | | | | | | | |
| rf:2789391 | 0.66 | 0.0228 | 0.0511 | 0.0179 | 0.61 | 0.0544 | 0.52 | 0.70 | 0.63 | 0.59 | 0.48 | 0.0666 | 0.65 | 0.0252 | 0.0213 | 0.040 | 0.011 |
| 2956442 | | | | | | | | | | | | | | | | | |
| knn:3066770 | 0.66 | 0.0233 | 0.0255 | 0.0143 | 0.59 | 0.0876 | 0.48 | 0.70 | 0.62 | 0.58 | 0.49 | 0.1126 | 0.69 | 0.0163 | 0.0178 | 0.028 | 0.008 |
| 2956442 | | | | | | | | | | | | | | | | | |
| knn:2332285 | 0.66 | 0.0234 | 0.0119 | 0.0041 | 0.67 | 0.0046 | 0.55 | 0.79 | 0.72 | 0.63 | 0.52 | 0.0047 | 0.71 | 0.0048 | 0.0063 | 0.044 | 0.014 |
| 3066770 | | | | | | | | | | | | | | | | | |
| rpart:2458376 | 0.66 | 0.0236 | 0.0255 | 0.0083 | 0.62 | 0.0320 | 0.61 | 0.64 | 0.63 | 0.62 | 0.50 | 0.0760 | 0.65 | 0.0227 | 0.0109 | 0.441 | 0.026 |
| 2512364 | | | | | | | | | | | | | | | | | |
| nb:3326487 | 0.66 | 0.0241 | 0.0505 | 0.0173 | 0.58 | 0.1339 | 0.24 | 0.91 | 0.73 | 0.55 | 0.57 | 0.0682 | 0.69 | 0.0078 | 0.0254 | 0.123 | 0.139 |
| 3463598 | | | | | | | | | | | | | | | | | |
| svm:2478155 | 0.66 | 0.0241 | 0.0505 | 0.0126 | 0.59 | 0.0876 | 0.42 | 0.76 | 0.64 | 0.57 | −0.18 | 0.1125 | 0.68 | 0.0107 | 0.0177 | 0.142 | 0.017 |
| 2651515 | | | | | | | | | | | | | | | | | |
| rf:2461837 | 0.66 | 0.0244 | 0.0966 | 0.0244 | 0.65 | 0.0093 | 0.55 | 0.76 | 0.69 | 0.63 | 0.53 | 0.0099 | 0.63 | 0.0335 | 0.0282 | 0.273 | 0.069 |
| 2651515 | | | | | | | | | | | | | | | | | |
| rf:2512364 | 0.66 | 0.0244 | 0.0119 | 0.0152 | 0.62 | 0.0320 | 0.61 | 0.64 | 0.63 | 0.62 | 0.53 | 0.0618 | 0.67 | 0.0164 | 0.0185 | 0.276 | 0.053 |
| 2364325 | | | | | | | | | | | | | | | | | |
| svm:2332285 | 0.66 | 0.0250 | 0.0247 | 0.0927 | 0.62 | 0.0320 | 0.55 | 0.70 | 0.64 | 0.61 | −0.14 | 0.0552 | 0.65 | 0.1287 | 0.1055 | 0.453 | 0.253 |
| 2478155 | | | | | | | | | | | | | | | | | |
| svm:2458376 | 0.66 | 0.0250 | 0.0048 | 0.0046 | 0.67 | 0.0046 | 0.48 | 0.85 | 0.76 | 0.62 | 0.15 | 0.0068 | 0.64 | 0.0159 | 0.0076 | 0.375 | 0.036 |
| 3463598 | | | | | | | | | | | | | | | | | |
| svm:2461837 | 0.66 | 0.0250 | 0.1729 | 0.0091 | 0.61 | 0.0544 | 0.64 | 0.58 | 0.60 | 0.61 | −0.50 | 0.0681 | 0.68 | 0.0067 | 0.0146 | 0.117 | 0.021 |
| 2651515 | | | | | | | | | | | | | | | | | |
| knn:3326487 | 0.66 | 0.0254 | 0.0255 | 0.0187 | 0.65 | 0.0093 | 0.55 | 0.76 | 0.69 | 0.63 | 0.48 | 0.0115 | 0.68 | 0.0171 | 0.0225 | 0.024 | 0.010 |
| 3139092 | | | | | | | | | | | | | | | | | |
| knn:3414753 | 0.66 | 0.0258 | 0.0255 | 0.0131 | 0.64 | 0.0178 | 0.55 | 0.73 | 0.67 | 0.62 | 0.50 | 0.0245 | 0.68 | 0.0176 | 0.0160 | 0.041 | 0.010 |
| 2956442 | | | | | | | | | | | | | | | | | |
| rpart:2332285 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 2789391 | | | | | | | | | | | | | | | | | |
| rpart:2478155 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 2789391 | | | | | | | | | | | | | | | | | |
| rpart:2789391 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 2364325 | | | | | | | | | | | | | | | | | |
| rpart:2789391 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 2512364 | | | | | | | | | | | | | | | | | |
| rpart:2789391 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 2956442 | | | | | | | | | | | | | | | | | |
| rpart:2789391 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 3005357 | | | | | | | | | | | | | | | | | |
| rpart:2789391 | 0.64 | 0.0258 | 0.1717 | 0.0242 | 0.64 | 0.0178 | 0.55 | 0.73 | 0.67 | 0.62 | 0.50 | 0.0278 | 0.61 | 0.0311 | 0.0266 | 0.050 | 0.018 |
| 3066770 | | | | | | | | | | | | | | | | | |
| rf:2458376 | 0.66 | 0.0261 | 0.0119 | 0.0128 | 0.68 | 0.0021 | 0.55 | 0.82 | 0.75 | 0.64 | 0.55 | 0.0058 | 0.64 | 0.0292 | 0.0167 | 0.412 | 0.019 |
| 2512364 | | | | | | | | | | | | | | | | | |
| rf:3463598 | 0.66 | 0.0264 | 0.0053 | 0.0011 | 0.68 | 0.0021 | 0.61 | 0.76 | 0.71 | 0.66 | 0.31 | 0.0013 | 0.71 | 0.0015 | 0.0028 | 0.100 | 0.019 |
| 2651515 | | | | | | | | | | | | | | | | | |
| nb:3463598 | 0.66 | 0.0267 | 0.0113 | 0.0049 | 0.67 | 0.0046 | 0.45 | 0.88 | 0.79 | 0.62 | 0.49 | 0.0010 | 0.69 | 0.0041 | 0.0094 | 0.156 | 0.024 |
| 2651515 | | | | | | | | | | | | | | | | | |
| svm:2478155 | 0.66 | 0.0267 | 0.0965 | 0.0258 | 0.61 | 0.0544 | 0.55 | 0.67 | 0.62 | 0.59 | 0.06 | 0.1274 | 0.68 | 0.0345 | 0.0291 | 0.045 | 0.010 |
| 2956441 | | | | | | | | | | | | | | | | | |
| svm:2789391 | 0.66 | 0.0267 | 0.0247 | 0.1214 | 0.65 | 0.0093 | 0.55 | 0.76 | 0.69 | 0.63 | −0.28 | 0.0123 | 0.64 | 0.1315 | 0.1387 | 0.441 | 0.099 |
| 3005357 | | | | | | | | | | | | | | | | | |
| rf:2461837 | 0.66 | 0.0274 | 0.0119 | 0.0104 | 0.65 | 0.0093 | 0.55 | 0.76 | 0.69 | 0.63 | 0.57 | 0.0149 | 0.64 | 0.0147 | 0.0130 | 0.104 | 0.023 |
| 3066770 | | | | | | | | | | | | | | | | | |
| svm:3326487 | 0.66 | 0.0276 | 0.0247 | 0.0294 | 0.55 | 0.2693 | 0.36 | 0.73 | 0.57 | 0.53 | −0.18 | 0.5564 | 0.68 | 0.0207 | 0.0353 | 0.147 | 0.214 |
| 341475 | | | | | | | | | | | | | | | | | |
| rf:3414753 | 0.66 | 0.0278 | 0.0255 | 0.0159 | 0.61 | 0.0544 | 0.45 | 0.76 | 0.65 | 0.58 | 0.57 | 0.1146 | 0.72 | 0.0250 | 0.0192 | 0.290 | 0.038 |
| 2512364 | | | | | | | | | | | | | | | | | |
| rf:3414753 | 0.66 | 0.0283 | 0.0255 | 0.0185 | 0.65 | 0.0093 | 0.73 | 0.58 | 0.63 | 0.68 | 0.52 | 0.0200 | 0.68 | 0.0303 | 0.0221 | 0.030 | 0.007 |
| 3139092 | | | | | | | | | | | | | | | | | |
| svm:2956442 | 0.66 | 0.0286 | 0.0113 | 0.0467 | 0.68 | 0.0021 | 0.76 | 0.61 | 0.66 | 0.71 | −0.28 | 0.0043 | 0.70 | 0.0538 | 0.0548 | 0.194 | 0.014 |
| 3005357 | | | | | | | | | | | | | | | | | |
| knn:3463598 | 0.66 | 0.0286 | 0.0511 | 0.0243 | 0.62 | 0.0320 | 0.45 | 0.79 | 0.68 | 0.59 | 0.54 | 0.0390 | 0.67 | 0.0255 | 0.0276 | 0.073 | 0.047 |
| 2461837 | | | | | | | | | | | | | | | | | |
| rf:2478155 | 0.66 | 0.0288 | 0.1717 | 0.0186 | 0.61 | 0.0544 | 0.36 | 0.85 | 0.71 | 0.57 | 0.58 | 0.0258 | 0.65 | 0.0135 | 0.0241 | 0.303 | 0.140 |
| 2651515 | | | | | | | | | | | | | | | | | |
| svm:2364325 | 0.66 | 0.0295 | 0.0965 | 0.0157 | 0.64 | 0.0178 | 0.36 | 0.91 | 0.80 | 0.59 | 0.25 | 0.0100 | 0.69 | 0.0169 | 0.0210 | 0.120 | 0.016 |
| 2651515 | | | | | | | | | | | | | | | | | |
| svm:2461837 | 0.66 | 0.0295 | 0.0019 | 0.0309 | 0.70 | 0.0009 | 0.64 | 0.76 | 0.72 | 0.68 | 0.07 | 0.0007 | 0.64 | 0.0336 | 0.0353 | 0.038 | 0.034 |
| 2364325 | | | | | | | | | | | | | | | | | |
| nb:3326487 | 0.65 | 0.0305 | 0.0965 | 0.0233 | 0.58 | 0.1339 | 0.33 | 0.82 | 0.65 | 0.55 | 0.47 | 0.1435 | 0.66 | 0.0106 | 0.0315 | 0.083 | 0.077 |
| 2789391 | | | | | | | | | | | | | | | | | |
| knn:2512364 | 0.65 | 0.0312 | 0.0119 | 0.0098 | 0.67 | 0.0046 | 0.55 | 0.79 | 0.72 | 0.63 | 0.54 | 0.0068 | 0.67 | 0.0152 | 0.0125 | 0.048 | 0.009 |
| 2956442 | | | | | | | | | | | | | | | | | |
| knn:2789391 | 0.65 | 0.0314 | 0.0511 | 0.0192 | 0.67 | 0.0046 | 0.52 | 0.82 | 0.74 | 0.63 | 0.54 | 0.0028 | 0.67 | 0.0230 | 0.0232 | 0.087 | 0.013 |
| 3463598 | | | | | | | | | | | | | | | | | |
| nb:2789391 | 0.65 | 0.0326 | 0.0048 | 0.0143 | 0.70 | 0.0009 | 0.70 | 0.70 | 0.70 | 0.70 | 0.43 | 0.0023 | 0.66 | 0.0127 | 0.0179 | 0.032 | 0.017 |
| 2461837 | | | | | | | | | | | | | | | | | |
| svm:2461837 | 0.65 | 0.0326 | 0.0505 | 0.0573 | 0.61 | 0.0544 | 0.48 | 0.73 | 0.64 | 0.59 | 0.05 | 0.0837 | 0.66 | 0.0578 | 0.0659 | 0.187 | 0.062 |
| 3066770 | | | | | | | | | | | | | | | | | |
| knn:2332285 | 0.65 | 0.0327 | 0.0053 | 0.0026 | 0.68 | 0.0021 | 0.52 | 0.85 | 0.77 | 0.64 | 0.52 | 0.0026 | 0.72 | 0.0035 | 0.0046 | 0.065 | 0.016 |
| 2651515 | | | | | | | | | | | | | | | | | |
| knn:2461837 | 0.65 | 0.0328 | 0.0255 | 0.0321 | 0.59 | 0.0876 | 0.48 | 0.70 | 0.62 | 0.58 | 0.53 | 0.1057 | 0.66 | 0.0366 | 0.0352 | 0.063 | 0.020 |
| 2956442 | | | | | | | | | | | | | | | | | |
| rpart:2364325 | 0.65 | 0.0335 | 0.0255 | 0.0254 | 0.59 | 0.0876 | 0.48 | 0.70 | 0.62 | 0.58 | 0.56 | 0.0866 | 0.66 | 0.0221 | 0.0282 | 0.004 | 0.004 |
| 3005357 | | | | | | | | | | | | | | | | | |
| nb:3066770 | 0.65 | 0.0337 | 0.0505 | 0.0257 | 0.62 | 0.0320 | 0.55 | 0.70 | 0.64 | 0.61 | 0.47 | 0.0365 | 0.68 | 0.0247 | 0.0298 | 0.076 | 0.033 |
| 2956442 | | | | | | | | | | | | | | | | | |
| svm:2458376 | 0.65 | 0.0337 | 0.0113 | 0.0092 | 0.67 | 0.0046 | 0.85 | 0.48 | 0.62 | 0.76 | 0.14 | 0.0095 | 0.65 | 0.0251 | 0.0137 | 0.026 | 0.003 |
| 3139092 | | | | | | | | | | | | | | | | | |
| svm:3066770 | 0.65 | 0.0337 | 0.0247 | 0.0078 | 0.64 | 0.0178 | 0.39 | 0.88 | 0.76 | 0.59 | −0.10 | 0.0043 | 0.64 | 0.0083 | 0.0129 | 0.383 | 0.039 |
| 2651515 | | | | | | | | | | | | | | | | | |
| svm:3414753 | 0.65 | 0.0348 | 0.0505 | 0.0204 | 0.61 | 0.0544 | 0.52 | 0.70 | 0.63 | 0.59 | 0.03 | 0.1041 | 0.68 | 0.0333 | 0.0234 | 0.113 | 0.020 |
| 2364325 | | | | | | | | | | | | | | | | | |
| rf:2458376 | 0.65 | 0.0360 | 0.0966 | 0.0439 | 0.59 | 0.0876 | 0.82 | 0.36 | 0.56 | 0.67 | 0.41 | 0.1245 | 0.62 | 0.0788 | 0.0484 | 0.117 | 0.019 |
| 2461837 | | | | | | | | | | | | | | | | | |
| svm:3463598 | 0.65 | 0.0372 | 0.0965 | 0.0380 | 0.62 | 0.0320 | 0.45 | 0.79 | 0.68 | 0.59 | 0.16 | 0.0386 | 0.68 | 0.0364 | 0.0441 | 0.324 | 0.023 |
| 2651515 | | | | | | | | | | | | | | | | | |
| rf:3414753 | 0.65 | 0.0383 | 0.0966 | 0.0215 | 0.64 | 0.0178 | 0.55 | 0.73 | 0.67 | 0.62 | 0.55 | 0.0310 | 0.68 | 0.0286 | 0.0246 | 0.150 | 0.040 |
| 3066770 | | | | | | | | | | | | | | | | | |
| knn:2458376 | 0.64 | 0.0388 | 0.6465 | 0.0344 | 0.59 | 0.0876 | 0.61 | 0.58 | 0.59 | 0.59 | 0.50 | 0.2430 | 0.59 | 0.0574 | 0.0381 | 0.160 | 0.056 |
| 2478155 | | | | | | | | | | | | | | | | | |
| rpart:3066770 | 0.62 | 0.0389 | 0.2868 | 0.0371 | 0.62 | 0.0320 | 0.45 | 0.79 | 0.68 | 0.59 | 0.50 | 0.0296 | 0.65 | 0.0334 | 0.0403 | 0.042 | 0.023 |
| 2956442 | | | | | | | | | | | | | | | | | |
| knn:2332285 | 0.65 | 0.0395 | 0.0119 | 0.0114 | 0.70 | 0.0009 | 0.64 | 0.76 | 0.72 | 0.68 | 0.50 | 0.0021 | 0.69 | 0.0133 | 0.0142 | 0.071 | 0.021 |
| 2461837 | | | | | | | | | | | | | | | | | |
| nb:3326487 | 0.65 | 0.0396 | 0.0505 | 0.0202 | 0.65 | 0.0093 | 0.61 | 0.70 | 0.67 | 0.64 | 0.39 | 0.0251 | 0.66 | 0.0110 | 0.0263 | 0.090 | 0.101 |
| 2461837 | | | | | | | | | | | | | | | | | |
| svm:2458376 | 0.65 | 0.0396 | 0.0965 | 0.0247 | 0.61 | 0.0544 | 0.42 | 0.79 | 0.67 | 0.58 | 0.15 | 0.1476 | 0.68 | 0.0605 | 0.0287 | 0.141 | 0.017 |
| 2956442 | | | | | | | | | | | | | | | | | |
| svm:2461837 | 0.65 | 0.0409 | 0.0505 | 0.0500 | 0.65 | 0.0093 | 0.55 | 0.76 | 0.69 | 0.63 | −0.07 | 0.0217 | 0.61 | 0.0816 | 0.0539 | 0.261 | 0.079 |
| 2512364 | | | | | | | | | | | | | | | | | |
| svm:3326487 | 0.65 | 0.0409 | 0.0505 | 0.0354 | 0.55 | 0.2693 | 0.36 | 0.73 | 0.57 | 0.53 | −0.32 | 0.5564 | 0.66 | 0.0176 | 0.0431 | 0.163 | 0.291 |
| 3463598 | | | | | | | | | | | | | | | | | |
| svm:3066770 | 0.65 | 0.0422 | 0.0965 | 0.0312 | 0.61 | 0.0544 | 0.55 | 0.67 | 0.62 | 0.59 | −0.18 | 0.0700 | 0.67 | 0.0292 | 0.0355 | 0.068 | 0.029 |
| 2956442 | | | | | | | | | | | | | | | | | |
| rpart:2512364 | 0.64 | 0.0435 | 0.4478 | 0.0595 | 0.58 | 0.1339 | 0.52 | 0.64 | 0.59 | 0.57 | 0.53 | 0.2051 | 0.65 | 0.0581 | 0.0616 | 0.046 | 0.017 |
| 3005357 | | | | | | | | | | | | | | | | | |
| rf:2461837 | 0.64 | 0.0441 | 0.0511 | 0.0243 | 0.65 | 0.0093 | 0.64 | 0.67 | 0.66 | 0.65 | 0.43 | 0.0270 | 0.64 | 0.0412 | 0.0272 | 0.103 | 0.025 |
| 2956442 | | | | | | | | | | | | | | | | | |
| nb:3463598 | 0.64 | 0.0449 | 0.0505 | 0.0297 | 0.67 | 0.0046 | 0.58 | 0.76 | 0.70 | 0.64 | 0.50 | 0.0093 | 0.69 | 0.0354 | 0.0337 | 0.086 | 0.026 |
| 2956442 | | | | | | | | | | | | | | | | | |
| rf:3326487 | 0.64 | 0.0454 | 0.0255 | 0.0086 | 0.67 | 0.0046 | 0.61 | 0.73 | 0.69 | 0.65 | 0.32 | 0.0113 | 0.69 | 0.0078 | 0.0127 | 0.147 | 0.066 |
| 3463598 | | | | | | | | | | | | | | | | | |
| rf:3139092 | 0.64 | 0.0454 | 0.0511 | 0.0445 | 0.62 | 0.0320 | 0.76 | 0.48 | 0.60 | 0.67 | 0.46 | 0.0497 | 0.66 | 0.0595 | 0.0482 | 0.033 | 0.006 |
| 2364325 | | | | | | | | | | | | | | | | | |
| rpart:2458376 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 3414753 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2332285 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2364325 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2478155 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2512364 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2789391 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 2956442 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 3005357 | | | | | | | | | | | | | | | | | |
| rpart:3414753 | 0.62 | 0.0463 | 0.286g | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 3066770 | | | | | | | | | | | | | | | | | |
| rpart:3414753 | 0.62 | 0.0463 | 0.2868 | 0.0446 | 0.62 | 0.0320 | 0.52 | 0.73 | 0.65 | 0.60 | 0.52 | 0.0514 | 0.66 | 0.0545 | 0.0467 | 0.203 | 0.046 |
| 3139092 | | | | | | | | | | | | | | | | | |
| rf:2461837 | 0.64 | 0.0475 | 0.0966 | 0.0350 | 0.64 | 0.0178 | 0.61 | 0.67 | 0.65 | 0.63 | 0.49 | 0.0522 | 0.63 | 0.0517 | 0.0383 | 0.255 | 0.043 |
| 2512364 | | | | | | | | | | | | | | | | | |
| svm:2461837 | 0.64 | 0.0478 | 0.1729 | 0.0447 | 0.55 | 0.2693 | 0.91 | 0.18 | 0.53 | 0.67 | −14.31 | 0.3448 | 0.59 | 0.1062 | 0.0572 | 0.255 | 0.065 |
| 2956442 | | | | | | | | | | | | | | | | | |
| rpart:2478155 | 0.64 | 0.0485 | 0.0966 | 0.1060 | 0.61 | 0.0544 | 0.58 | 0.64 | 0.61 | 0.60 | 0.56 | 0.1025 | 0.55 | 0.1274 | 0.1054 | 0.902 | 0.497 |
| 2512364 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2332285 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2364325 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2478155 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2512364 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2789391 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 2956442 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 3005357 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 3066770 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 3414753 | | | | | | | | | | | | | | | | | |
| rpart:3326487 | 0.58 | 0.0487 | 0.8432 | 0.0464 | 0.58 | 0.1339 | 0.18 | 0.97 | 0.86 | 0.54 | 0.57 | 0.0072 | 0.59 | 0.0101 | 0.0775 | 0.318 | 0.319 |
| 3463598 | | | | | | | | | | | | | | | | | |
| svm:2478155 | 0.64 | 0.0493 | 0.0247 | 0.0176 | 0.59 | 0.0876 | 0.64 | 0.55 | 0.58 | 0.60 | 0.32 | 0.1798 | 0.66 | 0.0330 | 0.0232 | 0.020 | 0.004 |
| 3139092 | | | | | | | | | | | | | | | | | |
| rf:3463598 | 0.64 | 0.0497 | 0.0119 | 0.0102 | 0.65 | 0.0093 | 0.45 | 0.85 | 0.75 | 0.61 | 0.60 | 0.0141 | 0.68 | 0.0159 | 0.0137 | 0.332 | 0.035 |
| 3066770 | | | | | | | | | | | | | | | | | |
| knn:2332285 | 0.64 | 0.0498 | 0.0053 | 0.0076 | 0.70 | 0.0009 | 0.61 | 0.79 | 0.74 | 0.67 | 0.51 | 0.0011 | 0.68 | 0.0117 | 0.0100 | 0.093 | 0.012 |
| 2478155 | | | | | | | | | | | | | | | | | |
| nb:2458376 | 0.80 | 0.0000 | 0.0001 | 0.0001 | 0.67 | 0.0046 | 0.45 | 0.88 | 0.79 | 0.62 | 0.50 | 0.0025 | 0.78 | 0.0003 | 0.0008 | 0.030 | 0.005 |
| 2478155 | | | | | | | | | | | | | | | | | |
| knn:2478155 | 0.79 | 0.0000 | 0.0009 | 0.0000 | 0.70 | 0.0009 | 0.61 | 0.79 | 0.74 | 0.67 | 0.53 | 0.0008 | 0.77 | 0.0001 | 0.0002 | 0.001 | 0.001 |
| 2364325 | | | | | | | | | | | | | | | | | |
| svm:2332285 | 0.79 | 0.0000 | 0.0001 | 0.0002 | 0.71 | 0.0004 | 0.64 | 0.79 | 0.75 | 0.68 | −0.14 | 0.0007 | 0.77 | 0.0006 | 0.0009 | 0.009 | 0.003 |
| 3005357 | | | | | | | | | | | | | | | | | |
| svm:2458376 | 0.78 | 0.0001 | 0.0007 | 0.0001 | 0.73 | 0.0001 | 0.67 | 0.79 | 0.76 | 0.70 | −0.24 | 0.0003 | 0.77 | 0.0004 | 0.0005 | 0.033 | 0.007 |
| 2332285 | | | | | | | | | | | | | | | | | |
| svm:2478155 | 0.78 | 0.0001 | 0.0000 | 0.0000 | 0.71 | 0.0004 | 0.70 | 0.73 | 0.72 | 0.71 | 0.19 | 0.0008 | 0.75 | 0.0003 | 0.0002 | 0.006 | 0.002 |
| 2364325 | | | | | | | | | | | | | | | | | |
| nb:2332285 | 0.78 | 0.0001 | 0.0001 | 0.0006 | 0.67 | 0.0046 | 0.55 | 0.79 | 0.72 | 0.63 | 0.50 | 0.0058 | 0.75 | 0.0009 | 0.0019 | 0.018 | 0.005 |
| 2478155 | | | | | | | | | | | | | | | | | |
| svm:2332285 | 0.78 | 0.0001 | 0.0007 | 0.0002 | 0.71 | 0.0004 | 0.64 | 0.79 | 0.75 | 0.68 | −0.12 | 0.0007 | 0.77 | 0.0005 | 0.0008 | 0.010 | 0.004 |
| 2512364 | | | | | | | | | | | | | | | | | |
| svm:2458376 | 0.78 | 0.0001 | 0.0002 | 0.0001 | 0.71 | 0.0004 | 0.61 | 0.82 | 0.77 | 0.68 | −0.10 | 0.0005 | 0.78 | 0.0005 | 0.0006 | 0.027 | 0.004 |
| 2364325 | | | | | | | | | | | | | | | | | |
| nb:3414753 | 0.78 | 0.0001 | 0.0002 | 0.0002 | 0.68 | 0.0021 | 0.61 | 0.76 | 0.71 | 0.66 | 0.49 | 0.0029 | 0.76 | 0.0002 | 0.0009 | 0.008 | 0.002 |
| 2478155 | | | | | | | | | | | | | | | | | |
| nb:2458376 | 0.77 | 0.0001 | 0.0002 | 0.0002 | 0.70 | 0.0009 | 0.61 | 0.79 | 0.74 | 0.67 | 0.45 | 0.0017 | 0.77 | 0.0005 | 0.0010 | 0.031 | 0.008 |
| 2364325 | | | | | | | | | | | | | | | | | |
| nb:2478155 | 0.77 | 0.0001 | 0.0007 | 0.0001 | 0.71 | 0.0004 | 0.73 | 0.70 | 0.71 | 0.72 | 0.44 | 0.0006 | 0.77 | 0.0001 | 0.0006 | 0.000 | 0.001 |
| 3005351 | | | | | | | | | | | | | | | | | |
| nb:2458376 | 0.77 | 0.0001 | 0.0007 | 0.0002 | 0.67 | 0.0046 | 0.52 | 0.82 | 0.74 | 0.63 | 0.50 | 0.0098 | 0.74 | 0.0007 | 0.0010 | 0.059 | 0.012 |
| 2332285 | | | | | | | | | | | | | | | | | |

Performance of pairwise combinations of the 15 markers across different metrics for the Recurrence endpoint. All classifiers have an Wilcox P-value <=0.05.

TABLE 12

| | | | | | | | | | UVA | UVA |
|---|---|---|---|---|---|---|---|---|---|---|
| | | WILCOX | KS | T-TEST | | | | KM | HR | OR |
| | | P- | P- | P- | | | | P- | P- | P- |
| CLASSIFIER | AUC | VALUE | VALUE | VALUE | PPV | NPV | CUTOFF | VALUE | VALUE | VALUE |
| rpart: 3326487 2458376 | 0.70 | 0.0172 | 0.2259 | 0.0114 | 0.50 | 0.82 | 0.41 | 0.0264 | 0.0061 | 0.0200 |
| nb: 3326487 3414753 | 0.70 | 0.0373 | 0.0330 | 0.0630 | 0.42 | 0.66 | 0.41 | 0.5225 | 0.0124 | 0.0872 |
| nb: 3326487 2332285 | 0.70 | 0.0373 | 0.0451 | 0.0412 | 0.54 | 0.71 | 0.37 | 0.0771 | 0.0088 | 0.0617 |
| nb: 3326487 2478155 | 0.73 | 0.0166 | 0.0192 | 0.0146 | 0.53 | 0.73 | 0.40 | 0.0456 | 0.0015 | 0.0343 |
| knn: 3326487 2478155 | 0.68 | 0.0287 | 0.1722 | 0.0256 | 0.56 | 0.78 | 0.50 | 0.0130 | 0.0197 | 0.0300 |
| knn: 3326487 3139092 | 0.69 | 0.0452 | 0.0453 | 0.0354 | 0.67 | 0.76 | 0.48 | 0.0051 | 0.0180 | 0.0448 |
| rpart: 3326487 3139092 | 0.67 | 0.0483 | 0.4356 | 0.0282 | 0.50 | 0.76 | 0.41 | 0.0643 | 0.0099 | 0.0380 |
| rf: 3326487 2789391 | 0.69 | 0.0466 | 0.0637 | 0.0266 | 0.56 | 0.76 | 0.33 | 0.0236 | 0.0071 | 0.0389 |
| knn: 3326487 2789391 | 0.71 | 0.0251 | 0.1494 | 0.0526 | 0.60 | 0.67 | 0.54 | 0.1140 | 0.0144 | 0.0736 |
| svm: 3326487 2789391 | 0.70 | 0.0373 | 0.0861 | 0.0460 | 0.50 | 0.72 | −0.44 | 0.1119 | 0.0173 | 0.0582 |
| rpart: 3326487 2461837 | 0.68 | 0.0287 | 0.1722 | 0.0256 | 0.56 | 0.78 | 0.47 | 0.0308 | 0.0405 | 0.0300 |
| nb: 3326487 2512364 | 0.70 | 0.0324 | 0.0546 | 0.0306 | 0.52 | 0.80 | 0.38 | 0.0339 | 0.0106 | 0.0444 |
| knn: 3326487 2512364 | 0.71 | 0.0233 | 0.0991 | 0.0391 | 0.45 | 0.67 | 0.50 | 0.4034 | 0.0216 | 0.0509 |
| nb: 3326487 2364325 | 0.69 | 0.0428 | 0.1781 | 0.0361 | 0.50 | 0.74 | 0.37 | 0.0894 | 0.0060 | 0.0595 |
| nb: 3326487 3066770 | 0.69 | 0.0458 | 0.0273 | 0.0729 | 0.56 | 0.69 | 0.41 | 0.1145 | 0.0184 | 0.0979 |
| rpart: 3326487 2651515 | 0.63 | 0.0399 | 0.5592 | 0.0362 | 0.71 | 0.71 | 0.56 | 0.0007 | 0.0025 | 0.0508 |
| rf: 2458376 3414753 | 0.75 | 0.0090 | 0.0721 | 0.0058 | 0.67 | 0.76 | 0.58 | 0.0038 | 0.0048 | 0.0114 |
| nb: 2458376 3414753 | 0.76 | 0.0055 | 0.0139 | 0.0027 | 0.75 | 0.73 | 0.48 | 0.0084 | 0.0006 | 0.0129 |
| svm: 2458376 3414753 | 0.72 | 0.0225 | 0.0652 | 0.0229 | 0.44 | 0.68 | 0.08 | 0.3676 | 0.0152 | 0.0305 |
| rf: 2458376 2332285 | 0.78 | 0.0023 | 0.0139 | 0.0025 | 0.46 | 0.85 | 0.30 | 0.0487 | 0.0025 | 0.0081 |
| nb: 2458376 2332285 | 0.77 | 0.0035 | 0.0497 | 0.0024 | 0.67 | 0.76 | 0.44 | 0.0039 | 0.0005 | 0.0111 |
| knn: 2458376 2332285 | 0.72 | 0.0212 | 0.0721 | 0.0170 | 0.56 | 0.76 | 0.50 | 0.0331 | 0.0097 | 0.0246 |
| rpart: 2458376 2332285 | 0.69 | 0.0216 | 0.1340 | 0.0187 | 0.52 | 0.83 | 0.48 | 0.0167 | 0.0273 | 0.0252 |
| svm: 2458376 2332285 | 0.79 | 0.0019 | 0.0243 | 0.0009 | 0.64 | 0.78 | −0.40 | 0.0074 | 0.0003 | 0.0058 |
| rf: 2458376 2478155 | 0.79 | 0.0024 | 0.0538 | 0.0010 | 0.50 | 0.87 | 0.49 | 0.0222 | 0.0030 | 0.0055 |
| nb: 2458376 2478155 | 0.86 | 0.0001 | 0.0002 | 0.0003 | 0.71 | 0.71 | 0.50 | 0.0101 | 0.0001 | 0.0068 |
| knn: 2458376 2478155 | 0.76 | 0.0050 | 0.0380 | 0.0024 | 0.80 | 0.77 | 0.83 | 0.0004 | 0.0051 | 0.0063 |
| rpart: 2458376 2478155 | 0.69 | 0.0216 | 0.1340 | 0.0187 | 0.52 | 0.83 | 0.48 | 0.0167 | 0.0273 | 0.0252 |
| svm: 2458376 2478155 | 0.80 | 0.0012 | 0.0069 | 0.0011 | 0.63 | 0.80 | 0.30 | 0.0042 | 0.0016 | 0.0043 |
| nb: 2458376 3139092 | 0.73 | 0.0166 | 0.0273 | 0.0038 | 0.86 | 0.74 | 0.49 | 0.0021 | 0.0012 | 0.0160 |
| rpart: 2458376 3139092 | 0.69 | 0.0216 | 0.1340 | 0.0187 | 0.52 | 0.83 | 0.48 | 0.0167 | 0.0273 | 0.0252 |
| rf: 2458376 2789391 | 0.76 | 0.0063 | 0.0062 | 0.0004 | 0.77 | 0.82 | 0.54 | 0.0001 | 0.0006 | 0.0028 |
| nb: 2458376 2789391 | 0.71 | 0.0243 | 0.0032 | 0.0029 | 0.70 | 0.74 | 0.49 | 0.0026 | 0.0006 | 0.0112 |
| rpart: 2458376 2789391 | 0.69 | 0.0216 | 0.1340 | 0.0187 | 0.52 | 0.83 | 0.48 | 0.0167 | 0.0273 | 0.0252 |
| rf: 2458376 3463598 | 0.73 | 0.0160 | 0.0317 | 0.0017 | 1.00 | 0.76 | 0.64 | 0.0000 | 0.0011 | 0.0072 |
| knn: 2458376 3463598 | 0.76 | 0.0062 | 0.0380 | 0.0004 | 0.86 | 0.74 | 0.53 | 0.0006 | 0.0004 | 0.0058 |
| rpart: 2458376 3463598 | 0.71 | 0.0043 | 0.0721 | 0.0028 | 0.73 | 0.77 | 0.53 | 0.0012 | 0.0032 | 0.0069 |
| rf: 2458376 2461837 | 0.70 | 0.0397 | 0.1112 | 0.0359 | 0.45 | 0.83 | 0.42 | 0.0728 | 0.0493 | 0.0454 |
| nb: 2458376 2461837 | 0.69 | 0.0489 | 0.0110 | 0.0101 | 0.45 | 0.74 | 0.37 | 0.2061 | 0.0049 | 0.0206 |
| knn: 2458376 2461837 | 0.69 | 0.0475 | 0.3486 | 0.0609 | 0.45 | 0.74 | 0.45 | 0.2061 | 0.0650 | 0.0662 |
| rpart: 2458376 2461837 | 0.68 | 0.0287 | 0.1722 | 0.0256 | 0.56 | 0.78 | 0.47 | 0.0308 | 0.0405 | 0.0300 |
| nb: 2458376 2512364 | 0.78 | 0.0021 | 0.0032 | 0.0014 | 0.75 | 0.73 | 0.52 | 0.0040 | 0.0008 | 0.0077 |
| knn: 2458376 2512364 | 0.74 | 0.0109 | 0.1154 | 0.0035 | 0.48 | 0.81 | 0.46 | 0.0656 | 0.0044 | 0.0100 |
| svm: 2458376 2512364 | 0.77 | 0.0031 | 0.0024 | 0.0008 | 0.69 | 0.79 | 0.19 | 0.0011 | 0.0007 | 0.0041 |
| rf: 2458376 2364325 | 0.80 | 0.0015 | 0.0076 | 0.0010 | 0.69 | 0.79 | 0.53 | 0.0008 | 0.0013 | 0.0038 |
| nb: 2458376 2364325 | 0.78 | 0.0026 | 0.0101 | 0.0020 | 0.62 | 0.75 | 0.45 | 0.0148 | 0.0006 | 0.0087 |
| knn: 2458376 2364325 | 0.74 | 0.0130 | 0.0991 | 0.0079 | 0.57 | 0.74 | 0.50 | 0.0377 | 0.0062 | 0.0140 |
| rpart: 2458376 2364325 | 0.69 | 0.0216 | 0.1340 | 0.0187 | 0.52 | 0.83 | 0.48 | 0.0167 | 0.0273 | 0.0252 |
| svm: 2458376 2364325 | 0.79 | 0.0017 | 0.0055 | 0.0016 | 0.62 | 0.75 | −0.21 | 0.0148 | 0.0012 | 0.0066 |
| rf: 2458376 3066770 | 0.73 | 0.0130 | 0.0451 | 0.0131 | 0.60 | 0.77 | 0.53 | 0.0097 | 0.0129 | 0.0210 |
| nb: 2458376 3066770 | 0.76 | 0.0046 | 0.0299 | 0.0020 | 0.70 | 0.74 | 0.45 | 0.0066 | 0.0003 | 0.0096 |
| knn: 2458376 3066770 | 0.73 | 0.0153 | 0.1494 | 0.0059 | 0.60 | 0.77 | 0.50 | 0.0082 | 0.0065 | 0.0128 |
| rpart: 2458376 3066770 | 0.69 | 0.0216 | 0.1340 | 0.0187 | 0.52 | 0.83 | 0.48 | 0.0167 | 0.0273 | 0.0252 |
| svm: 2458376 3066770 | 0.76 | 0.0046 | 0.0299 | 0.0029 | 0.64 | 0.78 | 0.13 | 0.0070 | 0.0056 | 0.0076 |
| rf: 2458376 2956442 | 0.70 | 0.0336 | 0.0637 | 0.0179 | 0.59 | 0.79 | 0.51 | 0.0096 | 0.0176 | 0.0245 |
| knn: 2458376 2956442 | 0.70 | 0.0326 | 0.1910 | 0.0173 | 0.54 | 0.71 | 0.52 | 0.0974 | 0.0127 | 0.0258 |
| rpart: 2458376 2956442 | 0.76 | 0.0051 | 0.0473 | 0.0131 | 0.60 | 0.77 | 0.50 | 0.0103 | 0.0124 | 0.0185 |
| rf: 2458376 3005357 | 0.74 | 0.0127 | 0.0453 | 0.0099 | 0.53 | 0.77 | 0.50 | 0.0300 | 0.0104 | 0.0158 |
| nb: 2458376 3005357 | 0.74 | 0.0093 | 0.0714 | 0.0039 | 0.75 | 0.73 | 0.50 | 0.0040 | 0.0015 | 0.0130 |
| knn: 2458376 3005357 | 0.74 | 0.0117 | 0.0751 | 0.0073 | 0.56 | 0.78 | 0.47 | 0.0192 | 0.0098 | 0.0125 |
| rpart: 2458376 3005357 | 0.69 | 0.0216 | 0.1340 | 0.0187 | 0.52 | 0.83 | 0.48 | 0.0167 | 0.0273 | 0.0252 |
| svm: 2458376 3005357 | 0.75 | 0.0072 | 0.0101 | 0.0053 | 0.67 | 0.72 | 0.11 | 0.0202 | 0.0063 | 0.0123 |
| nb: 2458376 2651515 | 0.70 | 0.0348 | 0.0497 | 0.0080 | 0.67 | 0.76 | 0.42 | 0.0030 | 0.0007 | 0.0211 |
| knn: 2458376 2651515 | 0.75 | 0.0073 | 0.0076 | 0.0036 | 0.41 | 0.75 | 0.37 | 0.2759 | 0.0013 | 0.0101 |
| rpart: 2458376 2651515 | 0.71 | 0.0161 | 0.1976 | 0.0117 | 0.52 | 0.80 | 0.39 | 0.0291 | 0.0032 | 0.0185 |
| svm: 2458376 2651515 | 0.70 | 0.0348 | 0.0714 | 0.0137 | 0.59 | 0.79 | −0.29 | 0.0101 | 0.0077 | 0.0207 |
| nb: 3414753 2332285 | 0.79 | 0.0017 | 0.0036 | 0.0038 | 0.67 | 0.76 | 0.45 | 0.0039 | 0.0010 | 0.0125 |
| svm: 3414753 2332285 | 0.76 | 0.0060 | 0.0299 | 0.0076 | 0.69 | 0.79 | −0.01 | 0.0007 | 0.0060 | 0.0128 |
| rf: 3414753 2478155 | 0.81 | 0.0013 | 0.0028 | 0.0015 | 0.56 | 1.00 | 0.28 | 0.0008 | 0.0027 | 0.0049 |
| nb: 3414753 2478155 | 0.83 | 0.0003 | 0.0005 | 0.0003 | 0.59 | 0.79 | 0.46 | 0.0069 | 0.0001 | 0.0031 |
| knn: 3414753 2478155 | 0.72 | 0.0213 | 0.1494 | 0.0200 | 0.50 | 0.76 | 0.45 | 0.0581 | 0.0107 | 0.0276 |
| svm: 3414753 2478155 | 0.81 | 0.0006 | 0.0014 | 0.0005 | 0.58 | 0.82 | −0.34 | 0.0062 | 0.0002 | 0.0035 |
| nb: 3414753 3139092 | 0.73 | 0.0130 | 0.0652 | 0.0055 | 0.56 | 0.78 | 0.43 | 0.0163 | 0.0017 | 0.0133 |
| svm: 3414753 3139092 | 0.72 | 0.0194 | 0.0177 | 0.0276 | 0.50 | 0.76 | −0.22 | 0.0666 | 0.0211 | 0.0332 |
| rf: 3414753 2789391 | 0.71 | 0.0255 | 0.0277 | 0.0161 | 0.63 | 0.80 | 0.50 | 0.0020 | 0.0143 | 0.0217 |
| nb: 3414753 2789391 | 0.73 | 0.0166 | 0.0177 | 0.0042 | 0.61 | 0.83 | 0.43 | 0.0039 | 0.0008 | 0.0114 |
| knn: 3414753 2789391 | 0.77 | 0.0034 | 0.0277 | 0.0010 | 0.67 | 0.81 | 0.46 | 0.0007 | 0.0006 | 0.0040 |
| svm: 3414753 2789391 | 0.72 | 0.0194 | 0.0177 | 0.0063 | 0.55 | 0.81 | −0.31 | 0.0177 | 0.0013 | 0.0143 |
| nb: 3414753 3463598 | 0.69 | 0.0428 | 0.0405 | 0.0086 | 0.64 | 0.78 | 0.46 | 0.0035 | 0.0022 | 0.0189 |
| knn: 3414753 3463598 | 0.71 | 0.0281 | 0.0453 | 0.0055 | 0.67 | 0.76 | 0.50 | 0.0064 | 0.0067 | 0.0118 |
| rpart: 3414753 3463598 | 0.71 | 0.0043 | 0.0721 | 0.0028 | 0.73 | 0.77 | 0.53 | 0.0012 | 0.0032 | 0.0069 |
| svm: 3414753 3463598 | 0.73 | 0.0130 | 0.0139 | 0.0051 | 0.63 | 0.80 | −0.14 | 0.0026 | 0.0047 | 0.0096 |
| nb: 3414753 2461837 | 0.71 | 0.0302 | 0.0221 | 0.0127 | 0.59 | 0.79 | 0.45 | 0.0141 | 0.0085 | 0.0199 |
| rpart: 3414753 2461837 | 0.72 | 0.0119 | 0.0721 | 0.0041 | 0.73 | 0.77 | 0.57 | 0.0039 | 0.0107 | 0.0083 |
| nb: 3414753 2512364 | 0.77 | 0.0031 | 0.0043 | 0.0013 | 0.57 | 0.85 | 0.44 | 0.0045 | 0.0010 | 0.0052 |
| knn: 3414753 2512364 | 0.77 | 0.0045 | 0.0332 | 0.0034 | 0.64 | 0.78 | 0.51 | 0.0030 | 0.0037 | 0.0079 |
| svm: 3414753 2512364 | 0.78 | 0.0026 | 0.0043 | 0.0007 | 0.60 | 0.86 | −0.19 | 0.0018 | 0.0003 | 0.0044 |
| rf: 3414753 2364325 | 0.71 | 0.0264 | 0.0164 | 0.0142 | 0.63 | 0.80 | 0.49 | 0.0026 | 0.0123 | 0.0199 |
| nb: 3414753 2364325 | 0.78 | 0.0028 | 0.0127 | 0.0024 | 0.58 | 0.82 | 0.45 | 0.0042 | 0.0010 | 0.0083 |
| knn: 3414753 2364325 | 0.71 | 0.0258 | 0.0847 | 0.0125 | 0.60 | 0.77 | 0.48 | 0.0056 | 0.0063 | 0.0190 |
| nb: 3414753 3066770 | 0.74 | 0.0093 | 0.0101 | 0.0025 | 0.64 | 0.73 | 0.47 | 0.0135 | 0.0005 | 0.0088 |
| knn: 3414753 3066770 | 0.74 | 0.0105 | 0.1910 | 0.0054 | 0.70 | 0.74 | 0.54 | 0.0030 | 0.0031 | 0.0126 |
| knn: 3414753 2956442 | 0.70 | 0.0310 | 0.0637 | 0.0313 | 0.54 | 0.71 | 0.50 | 0.0667 | 0.0161 | 0.0380 |
| svm: 3414753 2956442 | 0.70 | 0.0373 | 0.0451 | 0.0223 | 0.53 | 0.75 | −0.30 | 0.0546 | 0.0096 | 0.0304 |
| nb: 3414753 3005357 | 0.75 | 0.0086 | 0.0055 | 0.0045 | 0.61 | 0.83 | 0.43 | 0.0026 | 0.0022 | 0.0106 |
| knn: 3414753 3005357 | 0.78 | 0.0029 | 0.0115 | 0.0072 | 0.53 | 0.73 | 0.51 | 0.0503 | 0.0054 | 0.0127 |
| nb: 3414753 2651515 | 0.72 | 0.0179 | 0.0299 | 0.0038 | 0.56 | 0.76 | 0.41 | 0.0220 | 0.0001 | 0.0144 |
| rpart: 3414753 2651515 | 0.63 | 0.0399 | 0.5592 | 0.0362 | 0.71 | 0.71 | 0.56 | 0.0007 | 0.0025 | 0.0508 |
| rf: 2332285 2478155 | 0.81 | 0.0011 | 0.0199 | 0.0004 | 0.48 | 0.86 | 0.37 | 0.0343 | 0.0007 | 0.0040 |
| nb: 2332285 2478155 | 0.85 | 0.0001 | 0.0001 | 0.0008 | 0.67 | 0.76 | 0.47 | 0.0026 | 0.0002 | 0.0050 |
| rpart: 2332285 2478155 | 0.78 | 0.0017 | 0.0141 | 0.0013 | 0.53 | 0.77 | 0.55 | 0.0434 | 0.0029 | 0.0045 |
| rf: 2332285 3139092 | 0.73 | 0.0172 | 0.0062 | 0.0158 | 0.48 | 0.81 | 0.33 | 0.0656 | 0.0114 | 0.0237 |
| nb: 2332285 3139092 | 0.76 | 0.0046 | 0.0017 | 0.0034 | 0.67 | 0.76 | 0.48 | 0.0039 | 0.0011 | 0.0109 |
| knn: 2332285 3139092 | 0.70 | 0.0315 | 0.0076 | 0.0062 | 0.64 | 0.78 | 0.48 | 0.0025 | 0.0041 | 0.0111 |
| svm: 2332285 3139092 | 0.76 | 0.0050 | 0.0006 | 0.0059 | 0.52 | 0.80 | −0.26 | 0.0204 | 0.0049 | 0.0110 |
| rf: 2332285 2789391 | 0.76 | 0.0065 | 0.0034 | 0.0027 | 0.65 | 0.83 | 0.44 | 0.0010 | 0.0023 | 0.0068 |
| nb: 2332285 2789391 | 0.73 | 0.0153 | 0.0043 | 0.0056 | 0.67 | 0.76 | 0.47 | 0.0039 | 0.0014 | 0.0134 |
| knn: 2332285 2789391 | 0.78 | 0.0023 | 0.0052 | 0.0082 | 0.60 | 0.71 | 0.55 | 0.0299 | 0.0039 | 0.0147 |
| rpart: 2332285 2789391 | 0.73 | 0.0046 | 0.0332 | 0.0031 | 0.61 | 0.83 | 0.50 | 0.0031 | 0.0073 | 0.0061 |
| svm: 2332285 2789391 | 0.75 | 0.0072 | 0.0451 | 0.0056 | 0.67 | 0.76 | −0.01 | 0.0039 | 0.0022 | 0.0115 |
| rf: 2332285 3463598 | 0.75 | 0.0077 | 0.0134 | 0.0011 | 0.71 | 0.81 | 0.41 | 0.0003 | 0.0009 | 0.0045 |
| nb: 2332285 3463598 | 0.73 | 0.0166 | 0.0139 | 0.0051 | 0.75 | 0.79 | 0.47 | 0.0003 | 0.0015 | 0.0141 |
| knn: 2332285 3463598 | 0.75 | 0.0068 | 0.0637 | 0.0094 | 0.58 | 0.82 | 0.50 | 0.0087 | 0.0106 | 0.0196 |
| rpart: 2332285 3463598 | 0.71 | 0.0043 | 0.0721 | 0.0028 | 0.73 | 0.77 | 0.53 | 0.0012 | 0.0032 | 0.0069 |
| svm: 2332285 3463598 | 0.71 | 0.0243 | 0.0060 | 0.0014 | 0.75 | 0.79 | −0.14 | 0.0003 | 0.0018 | 0.0048 |
| rf: 2332285 2461837 | 0.73 | 0.0154 | 0.0380 | 0.0448 | 0.42 | 0.71 | 0.26 | 0.4266 | 0.0550 | 0.0511 |
| nb: 2332285 2461837 | 0.71 | 0.0261 | 0.0273 | 0.0104 | 0.60 | 0.77 | 0.46 | 0.0163 | 0.0049 | 0.0181 |
| rpart: 2332285 2461837 | 0.69 | 0.0409 | 0.1494 | 0.0578 | 0.45 | 0.71 | 0.38 | 0.3591 | 0.1003 | 0.0627 |
| svm: 2332285 2461837 | 0.77 | 0.0042 | 0.0243 | 0.0058 | 0.53 | 0.77 | −0.28 | 0.0447 | 0.0136 | 0.0105 |
| nb: 2332285 2512364 | 0.79 | 0.0015 | 0.0028 | 0.0021 | 0.67 | 0.76 | 0.51 | 0.0039 | 0.0011 | 0.0070 |
| svm: 2332285 2512364 | 0.80 | 0.0010 | 0.0008 | 0.0009 | 0.67 | 0.76 | −0.10 | 0.0039 | 0.0005 | 0.0046 |
| rf: 2332285 2364325 | 0.75 | 0.0083 | 0.0538 | 0.0042 | 0.64 | 0.78 | 0.49 | 0.0037 | 0.0031 | 0.0095 |
| nb: 2332285 2364325 | 0.77 | 0.0031 | 0.0299 | 0.0036 | 0.69 | 0.79 | 0.47 | 0.0005 | 0.0013 | 0.0105 |
| rpart: 2332285 2364325 | 0.69 | 0.0191 | 0.1154 | 0.0163 | 0.55 | 0.81 | 0.51 | 0.0131 | 0.0210 | 0.0212 |
| svm: 2332285 2364325 | 0.79 | 0.0017 | 0.0192 | 0.0023 | 0.67 | 0.76 | −0.02 | 0.0039 | 0.0014 | 0.0068 |
| nb: 2332285 3066770 | 0.75 | 0.0079 | 0.0101 | 0.0034 | 0.62 | 0.75 | 0.44 | 0.0135 | 0.0008 | 0.0117 |
| svm: 2332285 3066770 | 0.79 | 0.0019 | 0.0022 | 0.0020 | 0.67 | 0.76 | −0.25 | 0.0039 | 0.0011 | 0.0070 |
| svm: 2332285 2956442 | 0.76 | 0.0060 | 0.0157 | 0.0060 | 0.67 | 0.76 | −0.17 | 0.0039 | 0.0028 | 0.0129 |
| rf: 2332285 3005357 | 0.75 | 0.0090 | 0.0290 | 0.0104 | 0.48 | 0.78 | 0.30 | 0.0663 | 0.0056 | 0.0171 |
| nb: 2332285 3005357 | 0.76 | 0.0046 | 0.0028 | 0.0051 | 0.64 | 0.73 | 0.49 | 0.0123 | 0.0017 | 0.0131 |
| knn: 2332285 3005357 | 0.68 | 0.0493 | 0.1976 | 0.1346 | 0.52 | 0.80 | 0.39 | 0.0184 | 0.0971 | 0.1374 |
| rpart: 2332285 3005357 | 0.69 | 0.0333 | 0.3906 | 0.0381 | 0.53 | 0.75 | 0.50 | 0.0343 | 0.0206 | 0.0427 |
| svm: 2332285 3005357 | 0.81 | 0.0006 | 0.0022 | 0.0018 | 0.67 | 0.76 | −0.22 | 0.0039 | 0.0010 | 0.0068 |
| rf: 2332285 2651515 | 0.73 | 0.0143 | 0.0397 | 0.0052 | 0.73 | 0.77 | 0.44 | 0.0002 | 0.0008 | 0.0120 |
| nb: 2332285 2651515 | 0.76 | 0.0050 | 0.0060 | 0.0028 | 0.82 | 0.80 | 0.44 | 0.0000 | 0.0002 | 0.0105 |
| rpart: 2332285 2651515 | 0.63 | 0.0399 | 0.5592 | 0.0362 | 0.71 | 0.71 | 0.56 | 0.0007 | 0.0025 | 0.0508 |
| rf: 2478155 3139092 | 0.78 | 0.0030 | 0.0277 | 0.0033 | 0.63 | 0.80 | 0.66 | 0.0011 | 0.0047 | 0.0076 |
| nb: 2478155 3139092 | 0.82 | 0.0005 | 0.0017 | 0.0002 | 0.61 | 0.83 | 0.45 | 0.0013 | 0.0001 | 0.0025 |
| knn: 2478155 3139092 | 0.78 | 0.0033 | 0.0991 | 0.0025 | 0.55 | 0.81 | 0.48 | 0.0094 | 0.0052 | 0.0069 |
| svm: 2478155 3139092 | 0.71 | 0.0261 | 0.0393 | 0.0193 | 0.50 | 0.92 | −0.08 | 0.0100 | 0.0245 | 0.0290 |
| rf: 2478155 2789391 | 0.83 | 0.0005 | 0.0015 | 0.0003 | 0.55 | 0.81 | 0.47 | 0.0177 | 0.0008 | 0.0022 |
| nb: 2478155 2789391 | 0.79 | 0.0019 | 0.0079 | 0.0009 | 0.63 | 0.80 | 0.47 | 0.0020 | 0.0002 | 0.0045 |
| knn: 2478155 2789391 | 0.74 | 0.0100 | 0.0453 | 0.0163 | 0.69 | 0.79 | 0.54 | 0.0004 | 0.0067 | 0.0239 |
| rpart: 2478155 2789391 | 0.73 | 0.0046 | 0.0332 | 0.0031 | 0.61 | 0.83 | 0.50 | 0.0031 | 0.0073 | 0.0061 |
| svm: 2478155 2789391 | 0.74 | 0.0120 | 0.0060 | 0.0035 | 0.55 | 0.81 | −0.23 | 0.0177 | 0.0006 | 0.0097 |
| rf: 2478155 3463598 | 0.83 | 0.0006 | 0.0015 | 0.0005 | 0.50 | 1.00 | 0.27 | 0.0050 | 0.0014 | 0.0037 |
| nb: 2478155 3463598 | 0.76 | 0.0046 | 0.0367 | 0.0014 | 0.62 | 0.75 | 0.48 | 0.0072 | 0.0004 | 0.0065 |
| knn: 2478155 3463598 | 0.73 | 0.0130 | 0.0397 | 0.0103 | 0.52 | 0.83 | 0.44 | 0.0214 | 0.0138 | 0.0176 |
| rpart: 2478155 3463598 | 0.71 | 0.0043 | 0.0721 | 0.0028 | 0.73 | 0.77 | 0.53 | 0.0012 | 0.0032 | 0.0069 |
| svm: 2478155 3463598 | 0.79 | 0.0017 | 0.0101 | 0.0010 | 0.57 | 0.85 | 0.13 | 0.0088 | 0.0071 | 0.0045 |
| rf: 2478155 2461837 | 0.75 | 0.0094 | 0.0562 | 0.0033 | 0.60 | 0.77 | 0.64 | 0.0244 | 0.0127 | 0.0088 |
| nb: 2478155 2461837 | 0.77 | 0.0031 | 0.0079 | 0.0019 | 0.58 | 0.82 | 0.44 | 0.0080 | 0.0012 | 0.0060 |
| knn: 2478155 2461837 | 0.73 | 0.0159 | 0.0141 | 0.0010 | 0.54 | 1.00 | 0.42 | 0.0017 | 0.0068 | 0.0098 |
| rpart: 2478155 2461837 | 0.68 | 0.0287 | 0.1722 | 0.0256 | 0.56 | 0.78 | 0.47 | 0.0308 | 0.0405 | 0.0300 |
| svm: 2478155 2461837 | 0.71 | 0.0281 | 0.0221 | 0.0151 | 0.52 | 0.83 | −0.02 | 0.0242 | 0.0338 | 0.0221 |
| rf: 2478155 2512364 | 0.70 | 0.0359 | 0.0453 | 0.0374 | 0.46 | 0.80 | 0.43 | 0.1188 | 0.0465 | 0.0459 |
| nb: 2478155 2512364 | 0.83 | 0.0002 | 0.0014 | 0.0002 | 0.55 | 0.81 | 0.47 | 0.0094 | 0.0002 | 0.0023 |
| knn: 2478155 2512364 | 0.75 | 0.0074 | 0.0164 | 0.0037 | 0.58 | 0.82 | 0.50 | 0.0038 | 0.0026 | 0.0080 |
| rf: 2478155 2364325 | 0.86 | 0.0002 | 0.0003 | 0.0001 | 0.58 | 1.00 | 0.43 | 0.0004 | 0.0004 | 0.0018 |
| nb: 2478155 2364325 | 0.82 | 0.0004 | 0.0017 | 0.0002 | 0.58 | 0.82 | 0.46 | 0.0042 | 0.0001 | 0.0027 |
| knn: 2478155 2364325 | 0.83 | 0.0005 | 0.0052 | 0.0001 | 0.67 | 0.81 | 0.53 | 0.0003 | 0.0002 | 0.0012 |
| rpart: 2478155 2364325 | 0.68 | 0.0491 | 0.1199 | 0.0331 | 0.44 | 0.75 | 0.53 | 0.2860 | 0.0645 | 0.0425 |
| svm: 2478155 2364325 | 0.84 | 0.0002 | 0.0002 | 0.0002 | 0.65 | 0.90 | −0.01 | 0.0001 | 0.0006 | 0.0020 |
| rf: 2478155 3066770 | 0.79 | 0.0020 | 0.0080 | 0.0008 | 0.54 | 0.88 | 0.38 | 0.0035 | 0.0024 | 0.0040 |
| nb: 2478155 3066770 | 0.83 | 0.0003 | 0.0043 | 0.0002 | 0.65 | 0.83 | 0.44 | 0.0010 | 0.0000 | 0.0021 |
| knn: 2478155 3066770 | 0.79 | 0.0022 | 0.0172 | 0.0010 | 0.54 | 1.00 | 0.45 | 0.0017 | 0.0019 | 0.0059 |
| rpart: 2478155 3066770 | 0.76 | 0.0015 | 0.0141 | 0.0008 | 0.56 | 0.94 | 0.47 | 0.0013 | 0.0127 | 0.0078 |
| svm: 2478155 3066770 | 0.75 | 0.0079 | 0.0069 | 0.0097 | 0.57 | 0.89 | −0.03 | 0.0039 | 0.0131 | 0.0145 |
| rf: 2478155 2956442 | 0.77 | 0.0043 | 0.0172 | 0.0015 | 0.55 | 0.84 | 0.45 | 0.0077 | 0.0022 | 0.0058 |
| nb: 2478155 2956442 | 0.72 | 0.0194 | 0.0791 | 0.0097 | 0.50 | 0.76 | 0.43 | 0.0755 | 0.0032 | 0.0168 |
| knn: 2478155 2956442 | 0.73 | 0.0139 | 0.1783 | 0.0157 | 0.54 | 0.71 | 0.52 | 0.0697 | 0.0100 | 0.0218 |
| rpart: 2478155 2956442 | 0.76 | 0.0015 | 0.0141 | 0.0008 | 0.56 | 0.94 | 0.47 | 0.0013 | 0.0127 | 0.0078 |
| svm: 2478155 2956442 | 0.69 | 0.0428 | 0.1100 | 0.0449 | 0.47 | 0.73 | −0.18 | 0.1612 | 0.0257 | 0.0508 |
| rf: 2478155 3005357 | 0.77 | 0.0051 | 0.0277 | 0.0018 | 0.59 | 0.79 | 0.66 | 0.0031 | 0.0032 | 0.0057 |
| nb: 2478155 3005357 | 0.81 | 0.0008 | 0.0101 | 0.0006 | 0.58 | 0.82 | 0.44 | 0.0036 | 0.0002 | 0.0036 |
| knn: 2478155 3005357 | 0.79 | 0.0020 | 0.0380 | 0.0025 | 0.52 | 0.88 | 0.41 | 0.0065 | 0.0020 | 0.0081 |
| svm: 2478155 3005357 | 0.81 | 0.0009 | 0.0079 | 0.0011 | 0.58 | 0.82 | −0.07 | 0.0036 | 0.0013 | 0.0039 |
| nb: 2478155 2651515 | 0.80 | 0.0012 | 0.0032 | 0.0005 | 0.77 | 0.82 | 0.45 | 0.0000 | 0.0000 | 0.0048 |
| knn: 2478155 2651515 | 0.78 | 0.0035 | 0.0011 | 0.0002 | 0.63 | 0.80 | 0.46 | 0.0008 | 0.0001 | 0.0016 |
| rpart: 2478155 2651515 | 0.63 | 0.0399 | 0.5592 | 0.0362 | 0.71 | 0.71 | 0.56 | 0.0007 | 0.0025 | 0.0508 |
| svm: 2478155 2651515 | 0.69 | 0.0400 | 0.1175 | 0.0257 | 0.47 | 0.69 | −0.29 | 0.2121 | 0.0045 | 0.0352 |
| rf: 3139092 2789391 | 0.71 | 0.0246 | 0.0094 | 0.0141 | 0.59 | 0.79 | 0.53 | 0.0077 | 0.0128 | 0.0205 |
| nb: 3139092 2789391 | 0.73 | 0.0166 | 0.0139 | 0.0061 | 0.59 | 0.79 | 0.44 | 0.0077 | 0.0017 | 0.0135 |
| knn: 3139092 2789391 | 0.70 | 0.0363 | 0.0277 | 0.0068 | 0.63 | 0.80 | 0.48 | 0.0020 | 0.0045 | 0.0116 |
| rpart: 3139092 3463598 | 0.71 | 0.0043 | 0.0721 | 0.0028 | 0.73 | 0.77 | 0.53 | 0.0012 | 0.0032 | 0.0069 |
| nb: 3139092 2461837 | 0.69 | 0.0400 | 0.0221 | 0.0238 | 0.61 | 0.83 | 0.43 | 0.0042 | 0.0200 | 0.0311 |
| rpart: 3139092 2461837 | 0.68 | 0.0287 | 0.1722 | 0.0256 | 0.56 | 0.78 | 0.47 | 0.0308 | 0.0405 | 0.0300 |
| svm: 3139092 2461837 | 0.71 | 0.0243 | 0.0595 | 0.0230 | 0.53 | 0.77 | 0.02 | 0.0746 | 0.0377 | 0.0296 |
| nb: 3139092 2512364 | 0.75 | 0.0079 | 0.0017 | 0.0034 | 0.58 | 0.82 | 0.47 | 0.0050 | 0.0025 | 0.0085 |
| nb: 3139092 2364325 | 0.75 | 0.0072 | 0.0079 | 0.0051 | 0.58 | 0.82 | 0.44 | 0.0042 | 0.0024 | 0.0114 |
| knn: 3139092 2364325 | 0.72 | 0.0211 | 0.0637 | 0.0124 | 0.59 | 0.79 | 0.54 | 0.0052 | 0.0126 | 0.0174 |
| svm: 3139092 2364325 | 0.75 | 0.0079 | 0.0079 | 0.0070 | 0.55 | 0.81 | −0.15 | 0.0103 | 0.0051 | 0.0128 |
| nb: 3139092 3066770 | 0.74 | 0.0102 | 0.0101 | 0.0053 | 0.64 | 0.78 | 0.46 | 0.0041 | 0.0019 | 0.0124 |
| knn: 3139092 3066770 | 0.72 | 0.0215 | 0.0847 | 0.0105 | 0.64 | 0.78 | 0.51 | 0.0041 | 0.0103 | 0.0104 |
| svm: 3139092 3066770 | 0.72 | 0.0209 | 0.0177 | 0.0192 | 0.58 | 0.82 | −0.21 | 0.0056 | 0.0161 | 0.0260 |
| nb: 3139092 2956442 | 0.70 | 0.0348 | 0.0273 | 0.0432 | 0.53 | 0.77 | 0.43 | 0.0324 | 0.0241 | 0.0506 |
| knn: 3139092 2956442 | 0.70 | 0.0348 | 0.0332 | 0.0715 | 0.43 | 0.77 | 0.38 | 0.1932 | 0.0504 | 0.0784 |
| svm: 3139092 2956442 | 0.75 | 0.0066 | 0.0017 | 0.0351 | 0.50 | 0.79 | −0.04 | 0.0399 | 0.0237 | 0.0435 |
| nb: 3139092 3005357 | 0.72 | 0.0225 | 0.0022 | 0.0078 | 0.63 | 0.80 | 0.47 | 0.0022 | 0.0047 | 0.0142 |
| knn: 3139092 3005357 | 0.70 | 0.0320 | 0.0751 | 0.0244 | 0.52 | 0.83 | 0.41 | 0.0157 | 0.0155 | 0.0321 |
| svm: 3139092 3005357 | 0.72 | 0.0225 | 0.0101 | 0.0151 | 0.58 | 0.82 | 0.12 | 0.0050 | 0.0149 | 0.0220 |
| rf: 2789391 3463598 | 0.74 | 0.0104 | 0.0277 | 0.0034 | 0.67 | 0.81 | 0.43 | 0.0008 | 0.0029 | 0.0076 |
| nb: 2789391 3463598 | 0.71 | 0.0302 | 0.0194 | 0.0130 | 0.50 | 0.74 | 0.44 | 0.0782 | 0.0032 | 0.0218 |
| knn: 2789391 3463598 | 0.74 | 0.0117 | 0.0453 | 0.0062 | 0.69 | 0.79 | 0.54 | 0.0007 | 0.0056 | 0.0124 |
| rpart: 2789391 3463598 | 0.71 | 0.0043 | 0.0721 | 0.0028 | 0.73 | 0.77 | 0.53 | 0.0012 | 0.0032 | 0.0069 |
| svm: 2789391 3463598 | 0.71 | 0.0302 | 0.0139 | 0.0130 | 0.50 | 0.74 | −0.32 | 0.0782 | 0.0055 | 0.0200 |
| rf: 2789391 2461837 | 0.73 | 0.0143 | 0.0637 | 0.0105 | 0.56 | 0.78 | 0.47 | 0.0308 | 0.0166 | 0.0154 |
| nb: 2789391 2461837 | 0.69 | 0.0458 | 0.0221 | 0.0202 | 0.55 | 0.81 | 0.40 | 0.0177 | 0.0116 | 0.0273 |
| knn: 2789391 2461837 | 0.72 | 0.0192 | 0.0991 | 0.0147 | 0.60 | 0.77 | 0.49 | 0.0106 | 0.0108 | 0.0209 |
| rpart: 2789391 2461837 | 0.68 | 0.0287 | 0.1722 | 0.0256 | 0.56 | 0.78 | 0.47 | 0.0308 | 0.0405 | 0.0300 |
| svm: 2789391 2461837 | 0.69 | 0.0400 | 0.0177 | 0.0371 | 0.58 | 0.82 | −0.07 | 0.0103 | 0.0377 | 0.0437 |
| rf: 2789391 2512364 | 0.74 | 0.0113 | 0.0164 | 0.0030 | 0.61 | 0.83 | 0.48 | 0.0031 | 0.0044 | 0.0067 |
| nb: 2789391 2512364 | 0.76 | 0.0050 | 0.0101 | 0.0024 | 0.65 | 0.83 | 0.48 | 0.0013 | 0.0015 | 0.0068 |
| rpart: 2789391 2512364 | 0.73 | 0.0046 | 0.0332 | 0.0031 | 0.61 | 0.83 | 0.50 | 0.0031 | 0.0073 | 0.0061 |
| svm: 2789391 2512364 | 0.74 | 0.0093 | 0.0221 | 0.0086 | 0.59 | 0.79 | −0.33 | 0.0138 | 0.0104 | 0.0143 |
| rf: 2789391 2364325 | 0.77 | 0.0047 | 0.0290 | 0.0038 | 0.53 | 0.77 | 0.45 | 0.0338 | 0.0058 | 0.0081 |
| nb: 2789391 2364325 | 0.74 | 0.0102 | 0.0861 | 0.0045 | 0.56 | 0.78 | 0.47 | 0.0187 | 0.0017 | 0.0108 |
| knn: 2789391 2364325 | 0.78 | 0.0033 | 0.0847 | 0.0044 | 0.64 | 0.78 | 0.50 | 0.0030 | 0.0035 | 0.0090 |
| rpart: 2789391 2364325 | 0.73 | 0.0046 | 0.0332 | 0.0031 | 0.61 | 0.83 | 0.50 | 0.0031 | 0.0073 | 0.0061 |
| svm: 2789391 2364325 | 0.74 | 0.0111 | 0.0595 | 0.0052 | 0.53 | 0.77 | −0.26 | 0.0391 | 0.0016 | 0.0123 |
| rf: 2789391 3066770 | 0.75 | 0.0089 | 0.0062 | 0.0044 | 0.53 | 0.77 | 0.35 | 0.0338 | 0.0030 | 0.0088 |
| nb: 2789391 3066770 | 0.73 | 0.0153 | 0.0079 | 0.0053 | 0.56 | 0.78 | 0.43 | 0.0157 | 0.0010 | 0.0125 |
| knn: 2789391 3066770 | 0.78 | 0.0028 | 0.0229 | 0.0007 | 0.75 | 0.79 | 0.47 | 0.0002 | 0.0004 | 0.0039 |
| rpart: 2789391 3066770 | 0.73 | 0.0046 | 0.0332 | 0.0031 | 0.61 | 0.83 | 0.50 | 0.0031 | 0.0073 | 0.0061 |
| svm: 2789391 3066770 | 0.73 | 0.0130 | 0.0079 | 0.0048 | 0.56 | 0.78 | −0.30 | 0.0157 | 0.0026 | 0.0099 |
| rf: 2789391 2956442 | 0.75 | 0.0097 | 0.0473 | 0.0087 | 0.58 | 0.82 | 0.44 | 0.0080 | 0.0087 | 0.0138 |
| knn: 2789391 2956442 | 0.74 | 0.0122 | 0.1154 | 0.0109 | 0.53 | 0.73 | 0.50 | 0.0596 | 0.0055 | 0.0172 |
| rpart: 2789391 2956442 | 0.73 | 0.0046 | 0.0332 | 0.0031 | 0.61 | 0.83 | 0.50 | 0.0031 | 0.0073 | 0.0061 |
| svm: 2789391 2956442 | 0.70 | 0.0324 | 0.0079 | 0.0151 | 0.71 | 0.81 | 0.08 | 0.0003 | 0.0109 | 0.0218 |
| rf: 2789391 3005357 | 0.76 | 0.0074 | 0.0229 | 0.0072 | 0.52 | 0.80 | 0.41 | 0.0200 | 0.0066 | 0.0125 |
| nb: 2789391 3005357 | 0.74 | 0.0102 | 0.0330 | 0.0071 | 0.53 | 0.75 | 0.45 | 0.0411 | 0.0027 | 0.0132 |
| rpart: 2789391 3005357 | 0.73 | 0.0046 | 0.0332 | 0.0031 | 0.61 | 0.83 | 0.50 | 0.0031 | 0.0073 | 0.0061 |
| svm: 2789391 3005357 | 0.79 | 0.0017 | 0.0001 | 0.0644 | 0.68 | 0.91 | −0.28 | 0.0000 | 0.0473 | 0.1003 |
| rf: 2789391 2651515 | 0.75 | 0.0097 | 0.0751 | 0.0045 | 0.75 | 0.73 | 0.57 | 0.0005 | 0.0008 | 0.0118 |
| nb: 2789391 2651515 | 0.75 | 0.0079 | 0.0101 | 0.0035 | 0.63 | 0.80 | 0.43 | 0.0013 | 0.0001 | 0.0119 |
| knn: 2789391 2651515 | 0.76 | 0.0062 | 0.0453 | 0.0032 | 0.60 | 0.77 | 0.45 | 0.0077 | 0.0017 | 0.0076 |
| rpart: 2789391 2651515 | 0.63 | 0.0399 | 0.5592 | 0.0362 | 0.71 | 0.71 | 0.56 | 0.0007 | 0.0025 | 0.0508 |
| svm: 2789391 2651515 | 0.74 | 0.0111 | 0.0055 | 0.0072 | 0.59 | 0.79 | −0.38 | 0.0044 | 0.0008 | 0.0172 |
| rf: 3463598 2461837 | 0.74 | 0.0114 | 0.0277 | 0.0181 | 0.55 | 0.81 | 0.47 | 0.0140 | 0.0275 | 0.0247 |
| rpart: 3463598 2461837 | 0.68 | 0.0287 | 0.1722 | 0.0256 | 0.56 | 0.78 | 0.47 | 0.0308 | 0.0405 | 0.0300 |
| rf: 3463598 2512364 | 0.72 | 0.0230 | 0.0637 | 0.0100 | 0.75 | 0.73 | 0.62 | 0.0111 | 0.0124 | 0.0203 |
| nb: 3463598 2512364 | 0.75 | 0.0079 | 0.0060 | 0.0056 | 0.82 | 0.80 | 0.53 | 0.0000 | 0.0032 | 0.0122 |
| knn: 3463598 2512364 | 0.76 | 0.0061 | 0.0538 | 0.0048 | 0.67 | 0.69 | 0.61 | 0.1293 | 0.0067 | 0.0155 |
| rpart: 3463598 2512364 | 0.71 | 0.0043 | 0.0721 | 0.0028 | 0.73 | 0.77 | 0.53 | 0.0012 | 0.0032 | 0.0069 |
| rf: 3463598 2364325 | 0.72 | 0.0179 | 0.0110 | 0.0036 | 0.67 | 0.76 | 0.58 | 0.0056 | 0.0039 | 0.0086 |
| nb: 3463598 2364325 | 0.70 | 0.0348 | 0.0299 | 0.0129 | 0.60 | 0.77 | 0.48 | 0.0088 | 0.0059 | 0.0207 |
| knn: 3463598 2364325 | 0.73 | 0.0139 | 0.0847 | 0.0052 | 0.67 | 0.76 | 0.53 | 0.0060 | 0.0043 | 0.0103 |
| rpart: 3463598 2364325 | 0.71 | 0.0043 | 0.0721 | 0.0028 | 0.73 | 0.77 | 0.53 | 0.0012 | 0.0032 | 0.0069 |
| knn: 3463598 3066770 | 0.75 | 0.0075 | 0.0380 | 0.0014 | 0.73 | 0.77 | 0.48 | 0.0018 | 0.0016 | 0.0063 |
| rpart: 3463598 3066770 | 0.71 | 0.0043 | 0.0721 | 0.0028 | 0.73 | 0.77 | 0.53 | 0.0012 | 0.0032 | 0.0069 |
| svm: 3463598 3066770 | 0.69 | 0.0458 | 0.0139 | 0.0077 | 0.73 | 0.77 | −0.24 | 0.0018 | 0.0009 | 0.0188 |
| rf: 3463598 2956442 | 0.76 | 0.0065 | 0.0164 | 0.0053 | 0.55 | 0.81 | 0.42 | 0.0195 | 0.0109 | 0.0098 |
| knn: 3463598 2956442 | 0.70 | 0.0303 | 0.1291 | 0.0129 | 0.62 | 0.75 | 0.51 | 0.0162 | 0.0097 | 0.0195 |
| rpart: 3463598 2956442 | 0.69 | 0.0436 | 0.0991 | 0.0517 | 0.48 | 0.75 | 0.49 | 0.1720 | 0.0707 | 0.0565 |
| svm: 3463598 2956442 | 0.69 | 0.0400 | 0.1436 | 0.0239 | 0.58 | 0.72 | −0.04 | 0.0495 | 0.0170 | 0.0313 |
| rf: 3463598 3005357 | 0.70 | 0.0383 | 0.1722 | 0.0213 | 0.48 | 0.78 | 0.41 | 0.0663 | 0.0185 | 0.0278 |
| nb: 3463598 3005357 | 0.72 | 0.0209 | 0.0861 | 0.0148 | 0.53 | 0.77 | 0.44 | 0.0277 | 0.0075 | 0.0228 |
| rpart: 3463598 3005357 | 0.71 | 0.0043 | 0.0721 | 0.0028 | 0.73 | 0.77 | 0.53 | 0.0012 | 0.0032 | 0.0069 |
| rpart: 3463598 2651515 | 0.63 | 0.0399 | 0.5592 | 0.0362 | 0.71 | 0.71 | 0.56 | 0.0007 | 0.0025 | 0.0508 |
| nb: 2461837 2512364 | 0.74 | 0.0102 | 0.0221 | 0.0071 | 0.61 | 0.83 | 0.47 | 0.0042 | 0.0082 | 0.0125 |
| knn: 2461837 2512364 | 0.73 | 0.0139 | 0.0094 | 0.0155 | 0.48 | 0.81 | 0.48 | 0.0589 | 0.0220 | 0.0224 |
| rf: 2461837 2364325 | 0.73 | 0.0178 | 0.0751 | 0.0134 | 0.57 | 0.74 | 0.55 | 0.0511 | 0.0225 | 0.0188 |
| nb: 2461837 2364325 | 0.75 | 0.0079 | 0.0127 | 0.0105 | 0.56 | 0.78 | 0.43 | 0.0157 | 0.0082 | 0.0178 |
| knn: 2461837 2364325 | 0.74 | 0.0100 | 0.0453 | 0.0078 | 0.58 | 0.82 | 0.50 | 0.0042 | 0.0129 | 0.0138 |
| rpart: 2461837 2364325 | 0.68 | 0.0287 | 0.1722 | 0.0256 | 0.56 | 0.78 | 0.47 | 0.0308 | 0.0405 | 0.0300 |
| svm: 2461837 2364325 | 0.73 | 0.0130 | 0.0055 | 0.0264 | 0.63 | 0.80 | 0.07 | 0.0038 | 0.0351 | 0.0341 |
| nb: 2461837 3066770 | 0.70 | 0.0373 | 0.0127 | 0.0179 | 0.56 | 0.76 | 0.44 | 0.0264 | 0.0112 | 0.0254 |
| svm: 2461837 3066770 | 0.71 | 0.0243 | 0.0367 | 0.0274 | 0.54 | 0.71 | 0.15 | 0.1021 | 0.0236 | 0.0333 |
| rf: 2461837 2956442 | 0.70 | 0.0347 | 0.0991 | 0.0439 | 0.56 | 0.76 | 0.47 | 0.0465 | 0.0525 | 0.0494 |
| rpart: 2461837 2956442 | 0.68 | 0.0287 | 0.1722 | 0.0256 | 0.56 | 0.78 | 0.47 | 0.0308 | 0.0405 | 0.0300 |
| rf: 2461837 3005357 | 0.69 | 0.0423 | 0.0473 | 0.0446 | 0.50 | 0.74 | 0.46 | 0.0958 | 0.0522 | 0.0495 |
| nb: 2461837 3005357 | 0.74 | 0.0102 | 0.0055 | 0.0271 | 0.53 | 0.73 | 0.46 | 0.0651 | 0.0240 | 0.0333 |
| knn: 2461837 3005357 | 0.76 | 0.0063 | 0.0094 | 0.0025 | 0.60 | 0.86 | 0.44 | 0.0035 | 0.0068 | 0.0063 |
| rpart: 2461837 3005357 | 0.68 | 0.0287 | 0.1722 | 0.0256 | 0.56 | 0.78 | 0.47 | 0.0308 | 0.0405 | 0.0300 |
| svm: 2461837 3005357 | 0.71 | 0.0281 | 0.0127 | 0.0407 | 0.61 | 0.83 | 0.06 | 0.0042 | 0.0476 | 0.0467 |
| nb: 2461837 2651515 | 0.72 | 0.0194 | 0.0139 | 0.0117 | 0.53 | 0.77 | 0.41 | 0.0335 | 0.0026 | 0.0219 |
| nb: 2512364 2364325 | 0.79 | 0.0019 | 0.0017 | 0.0011 | 0.61 | 0.83 | 0.48 | 0.0015 | 0.0007 | 0.0042 |
| knn: 2512364 2364325 | 0.71 | 0.0265 | 0.2825 | 0.0277 | 0.60 | 0.71 | 0.70 | 0.0500 | 0.0377 | 0.0357 |
| rpart: 2512364 2364325 | 0.71 | 0.0117 | 0.0751 | 0.0093 | 0.55 | 0.84 | 0.48 | 0.0100 | 0.0188 | 0.0147 |
| svm: 2512364 2364325 | 0.76 | 0.0046 | 0.0017 | 0.0035 | 0.58 | 0.82 | −0.30 | 0.0042 | 0.0032 | 0.0082 |
| nb: 2512364 3066770 | 0.79 | 0.0017 | 0.0055 | 0.0015 | 0.65 | 0.83 | 0.47 | 0.0006 | 0.0013 | 0.0053 |
| knn: 2512364 3066770 | 0.80 | 0.0015 | 0.0241 | 0.0008 | 0.64 | 0.78 | 0.53 | 0.0054 | 0.0015 | 0.0043 |
| svm: 2512364 3066770 | 0.77 | 0.0035 | 0.0043 | 0.0041 | 0.60 | 0.86 | −0.01 | 0.0016 | 0.0062 | 0.0094 |
| nb: 2512364 2956442 | 0.71 | 0.0281 | 0.1100 | 0.0220 | 0.53 | 0.77 | 0.44 | 0.0324 | 0.0138 | 0.0286 |
| svm: 2512364 2956442 | 0.69 | 0.0400 | 0.1209 | 0.0289 | 0.57 | 0.74 | 0.05 | 0.0316 | 0.0172 | 0.0356 |
| nb: 2512364 3005357 | 0.76 | 0.0046 | 0.0177 | 0.0030 | 0.59 | 0.79 | 0.48 | 0.0049 | 0.0024 | 0.0073 |
| knn: 2512364 3005357 | 0.71 | 0.0293 | 0.0380 | 0.0479 | 0.42 | 0.68 | 0.50 | 0.4168 | 0.0312 | 0.0537 |
| nb: 2512364 2651515 | 0.77 | 0.0042 | 0.0079 | 0.0018 | 0.67 | 0.81 | 0.45 | 0.0007 | 0.0001 | 0.0092 |
| rpart: 2512364 2651515 | 0.63 | 0.0399 | 0.5592 | 0.0362 | 0.71 | 0.71 | 0.56 | 0.0007 | 0.0025 | 0.0508 |
| nb: 2364325 3066770 | 0.78 | 0.0023 | 0.0032 | 0.0019 | 0.50 | 0.82 | 0.40 | 0.0334 | 0.0004 | 0.0079 |
| knn: 2364325 3066770 | 0.76 | 0.0064 | 0.0110 | 0.0009 | 0.86 | 0.74 | 0.57 | 0.0001 | 0.0004 | 0.0069 |
| rpart: 2364325 3066770 | 0.69 | 0.0191 | 0.1154 | 0.0163 | 0.55 | 0.81 | 0.51 | 0.0131 | 0.0210 | 0.0212 |
| svm: 2364325 3066770 | 0.77 | 0.0031 | 0.0017 | 0.0015 | 0.52 | 0.83 | −0.44 | 0.0176 | 0.0011 | 0.0057 |
| rf: 2364325 2956442 | 0.72 | 0.0222 | 0.0277 | 0.0214 | 0.63 | 0.80 | 0.48 | 0.0032 | 0.0129 | 0.0279 |
| nb: 2364325 2956442 | 0.70 | 0.0324 | 0.0101 | 0.0322 | 0.64 | 0.78 | 0.48 | 0.0031 | 0.0175 | 0.0398 |
| knn: 2364325 2956442 | 0.71 | 0.0266 | 0.1154 | 0.0284 | 0.57 | 0.74 | 0.54 | 0.0249 | 0.0235 | 0.0347 |
| rpart: 2364325 2956442 | 0.69 | 0.0191 | 0.1154 | 0.0163 | 0.55 | 0.81 | 0.51 | 0.0131 | 0.0210 | 0.0212 |
| svm: 2364325 2956442 | 0.71 | 0.0302 | 0.0101 | 0.0245 | 0.62 | 0.75 | −0.09 | 0.0162 | 0.0172 | 0.0308 |
| nb: 2364325 3005357 | 0.75 | 0.0079 | 0.0101 | 0.0056 | 0.65 | 0.83 | 0.46 | 0.0007 | 0.0033 | 0.0112 |
| knn: 2364325 3005357 | 0.72 | 0.0179 | 0.0277 | 0.0100 | 0.61 | 0.83 | 0.50 | 0.0014 | 0.0076 | 0.0159 |
| svm: 2364325 3005357 | 0.74 | 0.0093 | 0.0101 | 0.0068 | 0.61 | 0.83 | 0.03 | 0.0016 | 0.0063 | 0.0124 |
| nb: 2364325 2651515 | 0.72 | 0.0225 | 0.0946 | 0.0069 | 0.53 | 0.75 | 0.43 | 0.0550 | 0.0006 | 0.0170 |
| knn: 2364325 2651515 | 0.71 | 0.0219 | 0.1783 | 0.0136 | 0.83 | 0.71 | 0.61 | 0.0000 | 0.0023 | 0.0272 |
| rpart: 2364325 2651515 | 0.63 | 0.0399 | 0.5592 | 0.0362 | 0.71 | 0.71 | 0.56 | 0.0007 | 0.0025 | 0.0508 |
| knn: 3066770 2956442 | 0.72 | 0.0188 | 0.1663 | 0.0214 | 0.50 | 0.70 | 0.49 | 0.1243 | 0.0129 | 0.0300 |
| rf: 3066770 3005357 | 0.72 | 0.0238 | 0.0751 | 0.0161 | 0.50 | 0.82 | 0.33 | 0.0265 | 0.0107 | 0.0227 |
| nb: 3066770 3005357 | 0.75 | 0.0072 | 0.0127 | 0.0042 | 0.58 | 0.72 | 0.48 | 0.0264 | 0.0019 | 0.0100 |
| knn: 3066770 3005357 | 0.75 | 0.0072 | 0.0164 | 0.0101 | 0.58 | 0.72 | 0.52 | 0.0264 | 0.0069 | 0.0156 |
| svm: 3066770 3005357 | 0.75 | 0.0079 | 0.0055 | 0.0069 | 0.61 | 0.83 | −0.07 | 0.0026 | 0.0066 | 0.0122 |
| nb: 3066770 2651515 | 0.74 | 0.0102 | 0.0079 | 0.0030 | 0.50 | 0.76 | 0.38 | 0.0414 | 0.0001 | 0.0132 |
| rpart: 3066770 2651515 | 0.63 | 0.0399 | 0.5592 | 0.0362 | 0.71 | 0.71 | 0.56 | 0.0007 | 0.0025 | 0.0508 |
| svm: 3066770 2651515 | 0.69 | 0.0458 | 0.1436 | 0.0118 | 0.53 | 0.75 | −0.63 | 0.0409 | 0.0018 | 0.0225 |
| rf: 2956442 2651515 | 0.72 | 0.0185 | 0.0453 | 0.0084 | 0.52 | 0.80 | 0.29 | 0.0163 | 0.0007 | 0.0200 |
| nb: 2956442 2651515 | 0.71 | 0.0261 | 0.0055 | 0.0275 | 0.50 | 0.82 | 0.37 | 0.0223 | 0.0040 | 0.0400 |
| knn: 2956442 2651515 | 0.77 | 0.0036 | 0.0241 | 0.0136 | 0.57 | 0.74 | 0.47 | 0.0253 | 0.0060 | 0.0216 |
| rpart: 2956442 2651515 | 0.63 | 0.0399 | 0.5592 | 0.0362 | 0.71 | 0.71 | 0.56 | 0.0007 | 0.0025 | 0.0508 |
| nb: 3005357 2651515 | 0.73 | 0.0141 | 0.0221 | 0.0049 | 0.52 | 0.83 | 0.38 | 0.0139 | 0.0003 | 0.0134 |
| rpart: 3005357 2651515 | 0.63 | 0.0399 | 0.5592 | 0.0362 | 0.71 | 0.71 | 0.56 | 0.0007 | 0.0025 | 0.0508 |
| svm: 3005357 2651515 | 0.69 | 0.0400 | 0.1367 | 0.0381 | 0.47 | 0.73 | −0.67 | 0.1165 | 0.0036 | 0.0531 |

Performance of pairwise combinations of the 15 markers across different metrics for the Recurrence endpoint for
LNI negative patients. All classifiers have a Wilcox P-value <= 0.05

Performance of pairwise combinations of the 15 markers across different metrics for the Recurrence endpoint for LNI negative patients. All classifiers have a Wilcox P-value <=0.05

TABLE 13

| | | | | | | | | | UVA | UVA |
|---|---|---|---|---|---|---|---|---|---|---|
| | | WILCOX | KS | T-TEST | | | | KM | HR | OR |
| | | P- | P- | P- | | | | P- | P- | P- |
| CLASSIFIER | AUC | VALUE | VALUE | VALUE | PPV | NPV | CUTOFF | VALUE | VALUE | VALUE |
| knn: 3326487 2478155 | 0.81 | 0.010 | 0.056 | 0.005 | 0.67 | 0.91 | 0.50 | 0.01 | 0.03 | 0.02 |
| rf: 3326487 3139092 | 0.79 | 0.043 | 0.128 | 0.051 | 0.10 | 0.40 | 0.49 | 0.02 | 0.05 | 0.07 |
| rf: 2458376 2512364 | 0.84 | 0.014 | 0.025 | 0.007 | 0.43 | 0.83 | 0.45 | 0.31 | 0.02 | 0.03 |
| nb: 2458376 2512364 | 0.78 | 0.046 | 0.036 | 0.021 | 0.50 | 0.88 | 0.41 | 0.10 | 0.04 | 0.05 |
| svm: 2458376 2512364 | 0.78 | 0.046 | 0.025 | 0.039 | 0.56 | 0.82 | −0.06 | 0.08 | 0.03 | 0.05 |
| nb: 2458376 2364325 | 0.79 | 0.037 | 0.137 | 0.047 | 0.50 | 0.80 | 0.42 | 0.16 | 0.07 | 0.06 |
| svm: 2458376 2364325 | 0.81 | 0.024 | 0.073 | 0.042 | 0.50 | 0.80 | −0.27 | 0.16 | 0.06 | 0.06 |
| nb: 2458376 3066770 | 0.79 | 0.037 | 0.203 | 0.033 | 0.44 | 0.73 | 0.39 | 0.44 | 0.05 | 0.05 |
| nb: 3414753 2512364 | 0.78 | 0.046 | 0.137 | 0.069 | 0.50 | 0.80 | 0.48 | 0.11 | 0.08 | 0.08 |
| rf: 2478155 2461837 | 0.79 | 0.043 | 0.115 | 0.074 | 0.43 | 0.83 | 0.36 | 0.30 | 0.13 | 0.09 |
| rf: 2478155 2512364 | 0.93 | 0.001 | 0.001 | 0.003 | 0.55 | 0.89 | 0.45 | 0.05 | 0.02 | 0.04 |
| rpart: 2478155 2512364 | 0.76 | 0.049 | 0.439 | 0.055 | 0.75 | 0.75 | 0.56 | 0.04 | 0.05 | 0.08 |
| rpart: 3139092 2512364 | 0.79 | 0.037 | 0.050 | 0.053 | 0.46 | 0.86 | 0.49 | 0.22 | 0.09 | 0.08 |
| rf: 2461837 2512364 | 0.82 | 0.019 | 0.025 | 0.008 | 0.56 | 0.82 | 0.51 | 0.15 | 0.04 | 0.02 |
| rpart: 2461837 2512364 | 0.76 | 0.045 | 0.115 | 0.015 | 0.71 | 0.85 | 0.63 | 0.03 | 0.06 | 0.03 |
| svm: 2461837 2512364 | 0.81 | 0.024 | 0.054 | 0.003 | 1.00 | 0.76 | 0.78 | 0.03 | 0.03 | 0.06 |
| rf: 2512364 2364325 | 0.78 | 0.047 | 0.115 | 0.044 | 0.55 | 0.89 | 0.44 | 0.04 | 0.04 | 0.06 |
| knn: 2512364 2364325 | 0.87 | 0.007 | 0.026 | 0.005 | 0.50 | 1.00 | 0.30 | 0.05 | 0.01 | 0.03 |
| svm: 2512364 3066770 | 0.80 | 0.030 | 0.046 | 0.049 | 0.57 | 0.77 | 0.37 | 0.08 | 0.06 | 0.06 |
| rf: 2512364 2956442 | 0.79 | 0.037 | 0.046 | 0.031 | 0.63 | 0.83 | 0.56 | 0.02 | 0.04 | 0.06 |
| rf: 2512364 3005357 | 0.84 | 0.014 | 0.025 | 0.015 | 0.60 | 0.90 | 0.51 | 0.01 | 0.02 | 0.04 |
| knn: 2512364 3005357 | 0.80 | 0.034 | 0.216 | 0.031 | 0.50 | 0.88 | 0.39 | 0.07 | 0.03 | 0.05 |
| rf: 2512364 2651515 | 0.85 | 0.014 | 0.026 | 0.014 | 0.57 | 0.77 | 0.65 | 0.09 | 0.02 | 0.03 |
| knn: 2512364 2651515 | 0.77 | 0.050 | 0.050 | 0.029 | 0.83 | 0.86 | 0.70 | 0.00 | 0.01 | 0.05 |
| svm: 2512364 2651515 | 0.79 | 0.037 | 0.073 | 0.046 | 0.71 | 0.85 | 0.25 | 0.00 | 0.04 | 0.06 |
| rpart: 3326487 2458376 | 0.77 | 0.044 | 0.238 | 0.036 | 0.17 | 0.38 | 0.51 | 0.04 | 0.05 | 0.05 |
| 3139092 | | | | | | | | | | |
| knn: 3326487 2512364 | 0.78 | 0.027 | 0.287 | 0.017 | 0.50 | 1.00 | 0.17 | 0.04 | 0.00 | 0.07 |
| 2651515 | | | | | | | | | | |
| rpart: 3326487 2364325 | 0.21 | 0.027 | 0.373 | 0.081 | 0.14 | 0.54 | 0.40 | 0.20 | 0.15 | 0.11 |
| 3066770 | | | | | | | | | | |
| nb: 2458376 3414753 | 0.78 | 0.046 | 0.137 | 0.032 | 0.55 | 0.89 | 0.41 | 0.05 | 0.05 | 0.05 |
| 2512364 | | | | | | | | | | |
| knn: 2458376 3414753 | 0.79 | 0.037 | 0.262 | 0.031 | 0.55 | 0.89 | 0.49 | 0.05 | 0.05 | 0.05 |
| 2512364 | | | | | | | | | | |
| svm: 2458376 3414753 | 0.79 | 0.037 | 0.054 | 0.030 | 0.50 | 0.80 | 0.25 | 0.20 | 0.05 | 0.05 |
| 2512364 | | | | | | | | | | |
| hdda: 2458376 3414753 | 0.78 | 0.046 | 0.094 | 0.035 | 0.75 | 0.75 | 0.53 | 0.06 | 0.06 | 0.06 |
| 2364325 | | | | | | | | | | |
| nb: 2458376 3414753 | 0.78 | 0.046 | 0.073 | 0.027 | 0.71 | 0.85 | 0.37 | 0.01 | 0.03 | 0.04 |
| 2651515 | | | | | | | | | | |
| rf: 2458376 2332285 | 0.85 | 0.011 | 0.012 | 0.017 | 0.46 | 0.86 | 0.41 | 0.18 | 0.03 | 0.04 |
| 2512364 | | | | | | | | | | |
| hdda: 2458376 2332285 | 0.80 | 0.030 | 0.036 | 0.052 | 0.67 | 0.79 | 0.47 | 0.04 | 0.05 | 0.07 |
| 2651515 | | | | | | | | | | |
| rf: 2458376 2478155 | 0.78 | 0.047 | 0.143 | 0.033 | 0.60 | 0.73 | 0.59 | 0.15 | 0.04 | 0.06 |
| 2789391 | | | | | | | | | | |
| svm: 2458376 2478155 | 0.82 | 0.019 | 0.094 | 0.016 | 0.56 | 0.82 | −0.04 | 0.10 | 0.03 | 0.04 |
| 2789391 | | | | | | | | | | |
| rf: 2458376 2478155 | 0.81 | 0.029 | 0.064 | 0.015 | 0.58 | 1.00 | 0.52 | 0.01 | 0.03 | 0.04 |
| 2512364 | | | | | | | | | | |
| knn: 2458376 2478155 | 0.78 | 0.045 | 0.115 | 0.039 | 0.55 | 0.89 | 0.43 | 0.05 | 0.07 | 0.06 |
| 3066770 | | | | | | | | | | |
| nb: 2458376 2478155 | 0.78 | 0.046 | 0.012 | 0.058 | 0.67 | 0.79 | 0.39 | 0.02 | 0.04 | 0.08 |
| 2651515 | | | | | | | | | | |
| knn: 2458376 2478155 | 0.80 | 0.030 | 0.238 | 0.019 | 0.63 | 0.83 | 0.50 | 0.05 | 0.03 | 0.04 |
| 2651515 | | | | | | | | | | |
| rf: 2458376 2789391 | 0.79 | 0.039 | 0.115 | 0.028 | 0.71 | 0.85 | 0.59 | 0.01 | 0.04 | 0.05 |
| 2512364 | | | | | | | | | | |
| rf: 2458376 3463598 | 0.80 | 0.030 | 0.012 | 0.025 | 0.43 | 0.83 | 0.39 | 0.33 | 0.03 | 0.08 |
| 2512364 | | | | | | | | | | |
| svm: 2458376 3463598 | 0.80 | 0.030 | 0.054 | 0.011 | 0.63 | 0.83 | −0.26 | 0.02 | 0.02 | 0.04 |
| 2512364 | | | | | | | | | | |
| rf: 2458376 2461837 | 0.79 | 0.039 | 0.115 | 0.019 | 0.71 | 0.85 | 0.59 | 0.02 | 0.05 | 0.05 |
| 2512364 | | | | | | | | | | |
| knn: 2458376 2461837 | 0.78 | 0.047 | 0.128 | 0.034 | 0.56 | 0.82 | 0.51 | 0.15 | 0.07 | 0.05 |
| 2512364 | | | | | | | | | | |
| rpart: 2458376 2461837 | 0.84 | 0.015 | 0.128 | 0.026 | 0.44 | 1.00 | 0.33 | 0.14 | 0.07 | 0.07 |
| 2364325 | | | | | | | | | | |
| rf: 2458376 2512364 | 0.84 | 0.016 | 0.022 | 0.010 | 0.80 | 0.80 | 0.68 | 0.01 | 0.01 | 0.03 |
| 2364325 | | | | | | | | | | |
| nb: 2458376 2512364 | 0.80 | 0.030 | 0.137 | 0.024 | 0.63 | 0.83 | 0.49 | 0.03 | 0.04 | 0.04 |
| 2364325 | | | | | | | | | | |
| svm: 2458376 2512364 | 0.88 | 0.005 | 0.017 | 0.009 | 0.67 | 0.79 | 0.39 | 0.04 | 0.02 | 0.03 |
| 2364325 | | | | | | | | | | |
| rf: 2458376 2512364 | 0.89 | 0.003 | 0.005 | 0.009 | 0.71 | 0.85 | 0.56 | 0.01 | 0.02 | 0.04 |
| 3066770 | | | | | | | | | | |
| nb: 2458376 2512364 | 0.79 | 0.037 | 0.137 | 0.029 | 0.50 | 0.80 | 0.39 | 0.13 | 0.05 | 0.05 |
| 3066770 | | | | | | | | | | |
| knn: 2458376 2512364 | 0.79 | 0.038 | 0.262 | 0.029 | 0.56 | 0.82 | 0.48 | 0.06 | 0.04 | 0.05 |
| 3066770 | | | | | | | | | | |
| svm: 2458376 2512364 | 0.82 | 0.019 | 0.094 | 0.011 | 0.57 | 0.77 | 0.28 | 0.20 | 0.03 | 0.03 |
| 3066770 | | | | | | | | | | |
| rf: 2458376 2512364 | 0.79 | 0.037 | 0.137 | 0.021 | 0.67 | 0.79 | 0.62 | 0.01 | 0.01 | 0.04 |
| 3005357 | | | | | | | | | | |
| nb: 2458376 2512364 | 0.78 | 0.046 | 0.169 | 0.028 | 0.55 | 0.89 | 0.38 | 0.06 | 0.05 | 0.05 |
| 3005357 | | | | | | | | | | |
| rpart: 2458376 2512364 | 0.77 | 0.044 | 0.439 | 0.039 | 0.56 | 0.82 | 0.50 | 0.05 | 0.05 | 0.05 |
| 3005357 | | | | | | | | | | |
| svm: 2458376 2512364 | 0.78 | 0.046 | 0.137 | 0.029 | 0.46 | 0.86 | −0.27 | 0.18 | 0.04 | 0.05 |
| 3005357 | | | | | | | | | | |
| rf: 2458376 2512364 | 0.82 | 0.022 | 0.022 | 0.019 | 0.80 | 0.80 | 0.66 | 0.00 | 0.02 | 0.04 |
| 2651515 | | | | | | | | | | |
| knn: 2458376 2512364 | 0.89 | 0.003 | 0.003 | 0.001 | 0.67 | 0.79 | 0.83 | 0.01 | 0.01 | 0.01 |
| 2651515 | | | | | | | | | | |
| nb: 2458376 2364325 | 0.78 | 0.046 | 0.137 | 0.061 | 0.50 | 0.80 | 0.35 | 0.16 | 0.03 | 0.08 |
| 2651515 | | | | | | | | | | |
| knn: 2458376 2364325 | 0.81 | 0.022 | 0.216 | 0.038 | 0.50 | 0.88 | 0.50 | 0.10 | 0.02 | 0.08 |
| 2651515 | | | | | | | | | | |
| nb: 2458376 3066770 | 0.78 | 0.046 | 0.111 | 0.048 | 0.57 | 0.77 | 0.42 | 0.14 | 0.07 | 0.06 |
| 3005357 | | | | | | | | | | |
| knn: 2458376 3066770 | 0.80 | 0.035 | 0.216 | 0.023 | 0.67 | 0.79 | 0.53 | 0.07 | 0.04 | 0.04 |
| 3005357 | | | | | | | | | | |
| nb: 2458376 3066770 | 0.80 | 0.030 | 0.073 | 0.021 | 0.67 | 0.79 | 0.37 | 0.04 | 0.02 | 0.04 |
| 2651515 | | | | | | | | | | |
| svm: 2458376 3066770 | 0.82 | 0.019 | 0.012 | 0.013 | 0.67 | 0.79 | 1.14 | 0.07 | 0.05 | 0.03 |
| 2651515 | | | | | | | | | | |
| hdda: 3414753 2478155 | 0.79 | 0.037 | 0.046 | 0.106 | 0.44 | 0.73 | 0.49 | 0.39 | 0.11 | 0.12 |
| 2512364 | | | | | | | | | | |
| svm: 3414753 2512364 | 0.78 | 0.046 | 0.073 | 0.065 | 0.50 | 0.80 | 0.27 | 0.13 | 0.05 | 0.08 |
| 2364325 | | | | | | | | | | |
| hdda: 3414753 2512364 | 0.78 | 0.046 | 0.111 | 0.100 | 0.44 | 0.73 | 0.49 | 0.39 | 0.11 | 0.11 |
| 3066770 | | | | | | | | | | |
| svm: 3414753 2512364 | 0.79 | 0.037 | 0.054 | 0.040 | 0.50 | 0.80 | −0.22 | 0.11 | 0.04 | 0.06 |
| 3066770 | | | | | | | | | | |
| rf: 3414753 2512364 | 0.79 | 0.039 | 0.143 | 0.065 | 0.57 | 0.77 | 0.66 | 0.12 | 0.06 | 0.08 |
| 2956442 | | | | | | | | | | |
| knn: 3414753 2512364 | 0.79 | 0.038 | 0.287 | 0.039 | 0.57 | 0.77 | 0.55 | 0.14 | 0.06 | 0.05 |
| 3005357 | | | | | | | | | | |
| rpart: 2332285 2478155 | 0.82 | 0.019 | 0.050 | 0.007 | 0.71 | 0.85 | 0.52 | 0.01 | 0.01 | 0.02 |
| 3463598 | | | | | | | | | | |
| rf: 2332285 2478155 | 0.84 | 0.016 | 0.022 | 0.013 | 0.55 | 0.89 | 0.46 | 0.06 | 0.03 | 0.04 |
| 2512364 | | | | | | | | | | |
| rf: 2332285 2512364 | 0.79 | 0.037 | 0.073 | 0.040 | 0.56 | 0.82 | 0.48 | 0.06 | 0.04 | 0.06 |
| 2364325 | | | | | | | | | | |
| rpart: 2332285 2512364 | 0.79 | 0.034 | 0.128 | 0.064 | 0.44 | 1.00 | 0.53 | 0.12 | 0.10 | 0.15 |
| 2364325 | | | | | | | | | | |
| rpart: 2332285 2512364 | 0.81 | 0.016 | 0.128 | 0.011 | 0.60 | 0.90 | 0.58 | 0.02 | 0.04 | 0.03 |
| 3066770 | | | | | | | | | | |
| rf: 2332285 2512364 | 0.79 | 0.039 | 0.128 | 0.058 | 0.56 | 0.82 | 0.52 | 0.06 | 0.06 | 0.07 |
| 3005357 | | | | | | | | | | |
| rf: 2478155 3463598 | 0.81 | 0.024 | 0.054 | 0.008 | 0.55 | 0.89 | 0.34 | 0.05 | 0.01 | 0.04 |
| 2512364 | | | | | | | | | | |
| rpart: 2478155 3463598 | 0.82 | 0.019 | 0.050 | 0.002 | 0.83 | 0.86 | 0.62 | 0.00 | 0.01 | 0.01 |
| 2512364 | | | | | | | | | | |
| rf: 2478155 2461837 | 0.86 | 0.008 | 0.012 | 0.012 | 0.46 | 0.86 | 0.36 | 0.18 | 0.04 | 0.04 |
| 2512364 | | | | | | | | | | |
| rf: 2478155 2512364 | 0.87 | 0.006 | 0.012 | 0.006 | 0.67 | 0.91 | 0.53 | 0.01 | 0.01 | 0.03 |
| 2364325 | | | | | | | | | | |
| svm: 2478155 2512364 | 0.78 | 0.046 | 0.111 | 0.034 | 0.57 | 0.77 | 0.26 | 0.07 | 0.02 | 0.05 |
| 2364325 | | | | | | | | | | |
| rf: 2478155 2512364 | 0.86 | 0.010 | 0.050 | 0.005 | 0.55 | 0.89 | 0.42 | 0.05 | 0.01 | 0.03 |
| 3066770 | | | | | | | | | | |
| rf: 2478155 2512364 | 0.80 | 0.030 | 0.094 | 0.022 | 0.60 | 0.90 | 0.42 | 0.02 | 0.03 | 0.04 |
| 2956442 | | | | | | | | | | |
| rf: 2478155 2512364 | 0.89 | 0.006 | 0.050 | 0.008 | 0.50 | 1.00 | 0.40 | 0.04 | 0.04 | 0.04 |
| 3005357 | | | | | | | | | | |
| rf: 2478155 2512364 | 0.84 | 0.017 | 0.050 | 0.023 | 0.56 | 0.82 | 0.50 | 0.08 | 0.03 | 0.04 |
| 2651515 | | | | | | | | | | |
| svm: 2478155 2364325 | 0.79 | 0.037 | 0.036 | 0.044 | 0.57 | 0.77 | 0.14 | 0.12 | 0.05 | 0.06 |
| 3066770 | | | | | | | | | | |
| svm: 2478155 2364325 | 0.78 | 0.046 | 0.046 | 0.080 | 0.63 | 0.83 | −0.01 | 0.02 | 0.06 | 0.09 |
| 3005357 | | | | | | | | | | |
| knn: 2478155 3066770 | 0.78 | 0.045 | 0.287 | 0.072 | 0.56 | 0.82 | 0.47 | 0.06 | 0.08 | 0.08 |
| 3005357 | | | | | | | | | | |
| knn: 3139092 2461837 | 0.85 | 0.012 | 0.019 | 0.013 | 0.43 | 0.83 | 0.36 | 0.33 | 0.02 | 0.04 |
| 2651515 | | | | | | | | | | |
| rpart: 3139092 2512364 | 0.78 | 0.044 | 0.050 | 0.037 | 0.63 | 0.83 | 0.56 | 0.03 | 0.05 | 0.05 |
| 3005357 | | | | | | | | | | |
| rf: 2789391 2461837 | 0.81 | 0.024 | 0.046 | 0.033 | 0.50 | 0.80 | 0.43 | 0.24 | 0.08 | 0.05 |
| 2512364 | | | | | | | | | | |
| rf: 2789391 2512364 | 0.80 | 0.030 | 0.017 | 0.054 | 0.50 | 0.71 | 0.66 | 0.28 | 0.06 | 0.07 |
| 2364325 | | | | | | | | | | |
| rf: 2789391 2512364 | 0.79 | 0.037 | 0.036 | 0.068 | 0.50 | 0.71 | 0.64 | 0.28 | 0.08 | 0.08 |
| 2956442 | | | | | | | | | | |
| svm: 2789391 3066770 | 0.78 | 0.046 | 0.036 | 0.106 | 0.60 | 0.73 | 0.13 | 0.07 | 0.06 | 0.12 |
| 2651515 | | | | | | | | | | |
| rf: 3463598 2461837 | 0.80 | 0.030 | 0.054 | 0.008 | 0.80 | 0.80 | 0.59 | 0.04 | 0.03 | 0.03 |
| 2512364 | | | | | | | | | | |
| rpart: 3463598 2461837 | 0.79 | 0.036 | 0.238 | 0.042 | 0.67 | 0.79 | 0.55 | 0.05 | 0.07 | 0.06 |
| 2956442 | | | | | | | | | | |
| rf: 3463598 2512364 | 0.79 | 0.037 | 0.025 | 0.023 | 0.46 | 0.86 | 0.33 | 0.13 | 0.02 | 0.06 |
| 3005357 | | | | | | | | | | |
| rf: 2461837 2512364 | 0.79 | 0.039 | 0.143 | 0.027 | 0.55 | 0.89 | 0.42 | 0.06 | 0.05 | 0.05 |
| 2364325 | | | | | | | | | | |
| svm: 2461837 2512364 | 0.80 | 0.030 | 0.094 | 0.036 | 0.56 | 0.82 | −0.12 | 0.12 | 0.06 | 0.05 |
| 3005357 | | | | | | | | | | |
| rf: 2461837 2512364 | 0.78 | 0.046 | 0.012 | 0.044 | 0.60 | 0.90 | 0.45 | 0.02 | 0.05 | 0.06 |
| 2651515 | | | | | | | | | | |
| rf: 2512364 2364325 | 0.80 | 0.030 | 0.036 | 0.036 | 0.60 | 0.73 | 0.67 | 0.09 | 0.03 | 0.05 |
| 3066770 | | | | | | | | | | |
| rf: 2512364 2364325 | 0.85 | 0.014 | 0.026 | 0.012 | 0.71 | 0.85 | 0.58 | 0.01 | 0.02 | 0.03 |
| 2956442 | | | | | | | | | | |
| rf: 2512364 2364325 | 0.79 | 0.037 | 0.073 | 0.037 | 0.55 | 0.89 | 0.48 | 0.03 | 0.03 | 0.05 |
| 3005357 | | | | | | | | | | |
| rf: 2512364 3066770 | 0.79 | 0.037 | 0.046 | 0.055 | 0.57 | 0.77 | 0.58 | 0.12 | 0.06 | 0.07 |
| 2956442 | | | | | | | | | | |
| knn: 2512364 3066770 | 0.79 | 0.040 | 0.128 | 0.051 | 0.63 | 0.83 | 0.51 | 0.03 | 0.06 | 0.06 |
| 3005357 | | | | | | | | | | |
| svm: 2512364 3066770 | 0.80 | 0.030 | 0.111 | 0.018 | 0.56 | 0.82 | 0.04 | 0.06 | 0.03 | 0.04 |
| 3005357 | | | | | | | | | | |
| rf: 2512364 2956442 | 0.79 | 0.043 | 0.128 | 0.112 | 0.60 | 0.90 | 0.35 | 0.02 | 0.06 | 0.13 |
| 2651515 | | | | | | | | | | |
| rpart: 2512364 2956442 | 0.89 | 0.005 | 0.022 | 0.002 | 0.50 | 1.00 | 0.33 | 0.05 | 0.03 | 0.03 |
| 2651515 | | | | | | | | | | |
| svm: 2512364 3005357 | 0.80 | 0.030 | 0.025 | 0.090 | 0.41 | 1.00 | −2.69 | 0.22 | 0.09 | 0.13 |
| 2651515 | | | | | | | | | | |
| knn: 3326487 2458376 | 0.79 | 0.038 | 0.128 | 0.094 | 0.57 | 0.77 | 0.47 | 0.14 | 0.11 | 0.11 |
| 2332285 3005357 | | | | | | | | | | |
| rpart: 3326487 3414753 | 0.21 | 0.027 | 0.373 | 0.081 | 0.14 | 0.54 | 0.40 | 0.20 | 0.15 | 0.11 |
| 2478155 3066770 | | | | | | | | | | |
| rf: 3326487 2478155 | 0.78 | 0.047 | 0.102 | 0.065 | 0.63 | 0.83 | 0.41 | 0.02 | 0.05 | 0.08 |
| 3463598 2512364 | | | | | | | | | | |
| svm: 3326487 2478155 | 0.78 | 0.046 | 0.036 | 0.209 | 0.40 | 0.80 | −0.13 | 0.51 | 0.27 | 0.23 |
| 3066770 2651515 | | | | | | | | | | |
| hdda: 3326487 3463598 | 0.82 | 0.019 | 0.046 | 0.010 | 0.23 | 0.43 | 0.46 | 0.10 | 0.01 | 0.04 |
| 2512364 3066770 | | | | | | | | | | |
| svm: 3326487 2512364 | 0.82 | 0.019 | 0.012 | 0.052 | 0.67 | 0.91 | −0.14 | 0.01 | 0.07 | 0.07 |
| 3066770 2651515 | | | | | | | | | | |
| hdda: 2458376 3414753 | 0.78 | 0.046 | 0.094 | 0.051 | 0.57 | 0.77 | 0.48 | 0.14 | 0.08 | 0.07 |
| 2332285 2364325 | | | | | | | | | | |
| svm: 2458376 3414753 | 0.79 | 0.037 | 0.094 | 0.065 | 0.57 | 0.77 | 0.08 | 0.14 | 0.09 | 0.08 |
| 2332285 2364325 | | | | | | | | | | |
| svm: 2458376 3414753 | 0.78 | 0.046 | 0.203 | 0.042 | 0.50 | 0.80 | 0.05 | 0.20 | 0.06 | 0.06 |
| 2478155 2512364 | | | | | | | | | | |
| nb: 2458376 3414753 | 0.78 | 0.046 | 0.046 | 0.079 | 0.57 | 0.77 | 0.40 | 0.12 | 0.08 | 0.09 |
| 2478155 3066770 | | | | | | | | | | |
| rpart: 2458376 3414753 | 0.78 | 0.043 | 0.216 | 0.069 | 0.55 | 0.89 | 0.59 | 0.06 | 0.11 | 0.09 |
| 3139092 2512364 | | | | | | | | | | |
| hdda: 2458376 3414753 | 0.78 | 0.046 | 0.094 | 0.031 | 0.75 | 0.75 | 0.53 | 0.06 | 0.05 | 0.06 |
| 3463598 2364325 | | | | | | | | | | |
| rpart: 2458376 3414753 | 0.84 | 0.013 | 0.056 | 0.004 | 0.67 | 0.91 | 0.55 | 0.01 | 0.04 | 0.02 |
| 2461837 2512364 | | | | | | | | | | |
| hdda: 2458376 3414753 | 0.80 | 0.030 | 0.094 | 0.036 | 0.75 | 0.75 | 0.53 | 0.06 | 0.06 | 0.06 |
| 2512364 2364325 | | | | | | | | | | |
| nb: 2458376 3414753 | 0.80 | 0.030 | 0.111 | 0.038 | 0.63 | 0.83 | 0.46 | 0.03 | 0.05 | 0.05 |
| 2512364 2364325 | | | | | | | | | | |
| svm: 2458376 3414753 | 0.80 | 0.030 | 0.073 | 0.028 | 0.56 | 0.82 | 0.38 | 0.06 | 0.03 | 0.04 |
| 2512364 2364325 | | | | | | | | | | |
| svm: 2458376 3414753 | 0.84 | 0.014 | 0.054 | 0.004 | 0.54 | 1.00 | −0.29 | 0.02 | 0.01 | 0.03 |
| 2512364 3066770 | | | | | | | | | | |
| rpart: 2458376 3414753 | 0.77 | 0.044 | 0.439 | 0.039 | 0.56 | 0.82 | 0.50 | 0.05 | 0.05 | 0.05 |
| 2512364 3005357 | | | | | | | | | | |
| rf: 2458376 3414753 | 0.83 | 0.019 | 0.115 | 0.024 | 0.56 | 0.82 | 0.54 | 0.04 | 0.03 | 0.05 |
| 2512364 2651515 | | | | | | | | | | |
| nb: 2458376 3414753 | 0.80 | 0.030 | 0.073 | 0.020 | 0.63 | 0.83 | 0.38 | 0.03 | 0.03 | 0.04 |
| 2512364 2651515 | | | | | | | | | | |
| nb: 2458376 3414753 | 0.78 | 0.046 | 0.094 | 0.073 | 0.57 | 0.77 | 0.45 | 0.14 | 0.08 | 0.08 |
| 2364325 3066770 | | | | | | | | | | |
| nb: 2458376 3414753 | 0.81 | 0.024 | 0.046 | 0.028 | 0.63 | 0.83 | 0.34 | 0.03 | 0.04 | 0.04 |
| 3066770 2651515 | | | | | | | | | | |
| rf: 2458376 2332285 | 0.85 | 0.014 | 0.008 | 0.014 | 0.55 | 0.89 | 0.49 | 0.05 | 0.03 | 0.04 |
| 2478155 2512364 | | | | | | | | | | |
| svm: 2458376 2332285 | 0.78 | 0.046 | 0.094 | 0.063 | 0.57 | 0.77 | 0.01 | 0.14 | 0.09 | 0.07 |
| 2478155 2364325 | | | | | | | | | | |
| rpart: 2458376 2332285 | 0.84 | 0.015 | 0.128 | 0.025 | 0.41 | 1.00 | 0.23 | 0.20 | 0.08 | 0.05 |
| 3139092 2461837 | | | | | | | | | | |
| rf: 2458376 2332285 | 0.78 | 0.046 | 0.094 | 0.041 | 0.57 | 0.77 | 0.59 | 0.14 | 0.07 | 0.06 |
| 2789391 2512364 | | | | | | | | | | |
| rf: 2458376 2332285 | 0.78 | 0.046 | 0.054 | 0.028 | 0.46 | 0.86 | 0.38 | 0.20 | 0.03 | 0.06 |
| 3463598 2512364 | | | | | | | | | | |
| hdda: 2458376 2332285 | 0.78 | 0.046 | 0.094 | 0.057 | 0.50 | 0.71 | 0.47 | 0.31 | 0.05 | 0.08 |
| 3463598 2651515 | | | | | | | | | | |
| hdda: 2458376 2332285 | 0.78 | 0.046 | 0.036 | 0.066 | 0.50 | 0.88 | 0.42 | 0.13 | 0.06 | 0.08 |
| 2461837 2651515 | | | | | | | | | | |
| rf: 2458376 2332285 | 0.81 | 0.024 | 0.036 | 0.022 | 0.63 | 0.83 | 0.59 | 0.03 | 0.04 | 0.04 |
| 2512364 2364325 | | | | | | | | | | |
| svm: 2458376 2332285 | 0.79 | 0.037 | 0.111 | 0.036 | 0.56 | 0.82 | 0.18 | 0.06 | 0.05 | 0.05 |
| 2512364 2364325 | | | | | | | | | | |
| nb: 2458376 2332285 | 0.78 | 0.046 | 0.094 | 0.095 | 0.50 | 0.75 | 0.41 | 0.25 | 0.12 | 0.10 |
| 2512364 3066770 | | | | | | | | | | |
| rf: 2458376 2332285 | 0.82 | 0.019 | 0.025 | 0.015 | 0.56 | 0.82 | 0.56 | 0.06 | 0.03 | 0.04 |
| 2512364 3005357 | | | | | | | | | | |
| rf: 2458376 2332285 | 0.86 | 0.011 | 0.022 | 0.019 | 0.75 | 0.92 | 0.55 | 0.00 | 0.03 | 0.04 |
| 2512364 2651515 | | | | | | | | | | |
| hdda: 2458376 2332285 | 0.78 | 0.046 | 0.169 | 0.058 | 0.50 | 0.88 | 0.42 | 0.13 | 0.05 | 0.08 |
| 2364325 2651515 | | | | | | | | | | |
| svm: 2458376 2332285 | 0.78 | 0.046 | 0.046 | 0.090 | 0.50 | 0.75 | −0.29 | 0.25 | 0.11 | 0.10 |
| 3066770 3005357 | | | | | | | | | | |
| hdda: 2458376 2332285 | 0.78 | 0.046 | 0.073 | 0.041 | 0.75 | 0.75 | 0.50 | 0.02 | 0.04 | 0.06 |
| 2956442 2651515 | | | | | | | | | | |
| rf: 2458376 2478155 | 0.84 | 0.014 | 0.036 | 0.010 | 0.47 | 1.00 | 0.40 | 0.07 | 0.02 | 0.05 |
| 2789391 2512364 | | | | | | | | | | |
| knn: 2458376 2478155 | 0.81 | 0.025 | 0.022 | 0.040 | 0.75 | 0.92 | 0.50 | 0.00 | 0.07 | 0.06 |
| 2461837 2651515 | | | | | | | | | | |
| rf: 2458376 2478155 | 0.85 | 0.011 | 0.012 | 0.012 | 0.50 | 1.00 | 0.43 | 0.04 | 0.02 | 0.04 |
| 2512364 2364325 | | | | | | | | | | |
| nb: 2458376 2478155 | 0.79 | 0.037 | 0.073 | 0.039 | 0.56 | 0.82 | 0.38 | 0.06 | 0.04 | 0.05 |
| 2512364 2364325 | | | | | | | | | | |
| svm: 2458376 2478155 | 0.80 | 0.030 | 0.111 | 0.036 | 0.57 | 0.77 | 0.24 | 0.16 | 0.06 | 0.06 |
| 2512364 2364325 | | | | | | | | | | |
| rf: 2458376 2478155 | 0.85 | 0.012 | 0.056 | 0.006 | 0.47 | 1.00 | 0.36 | 0.07 | 0.01 | 0.03 |
| 2512364 3066770 | | | | | | | | | | |
| nb: 2458376 2478155 | 0.79 | 0.037 | 0.137 | 0.047 | 0.50 | 0.75 | 0.39 | 0.25 | 0.05 | 0.06 |
| 2512364 3066770 | | | | | | | | | | |
| knn: 2458376 2478155 | 0.80 | 0.029 | 0.056 | 0.016 | 0.67 | 0.91 | 0.50 | 0.01 | 0.05 | 0.03 |
| 2512364 3066770 | | | | | | | | | | |
| svm: 2458376 2478155 | 0.89 | 0.003 | 0.017 | 0.003 | 0.63 | 0.83 | 0.16 | 0.05 | 0.02 | 0.02 |
| 2512364 3066770 | | | | | | | | | | |
| rf: 2458376 2478155 | 0.78 | 0.046 | 0.137 | 0.029 | 0.46 | 0.86 | 0.43 | 0.18 | 0.04 | 0.05 |
| 2512364 2956442 | | | | | | | | | | |
| rf: 2458376 2478155 | 0.82 | 0.022 | 0.056 | 0.019 | 0.47 | 1.00 | 0.33 | 0.07 | 0.02 | 0.05 |
| 2512364 3005357 | | | | | | | | | | |
| rf: 2458376 2478155 | 0.82 | 0.019 | 0.036 | 0.016 | 0.55 | 0.89 | 0.50 | 0.04 | 0.03 | 0.04 |
| 2512364 2651515 | | | | | | | | | | |
| knn: 2458376 2478155 | 0.78 | 0.046 | 0.262 | 0.049 | 0.67 | 0.79 | 0.55 | 0.02 | 0.04 | 0.06 |
| 2512364 2651515 | | | | | | | | | | |
| knn: 2458376 2478155 | 0.79 | 0.042 | 0.238 | 0.046 | 0.63 | 0.83 | 0.48 | 0.03 | 0.03 | 0.06 |
| 2364325 2651515 | | | | | | | | | | |
| svm: 2458376 2478155 | 0.78 | 0.046 | 0.073 | 0.045 | 0.67 | 0.79 | −0.16 | 0.04 | 0.04 | 0.06 |
| 2364325 2651515 | | | | | | | | | | |
| nb: 2458376 2478155 | 0.79 | 0.037 | 0.111 | 0.062 | 0.50 | 0.75 | 0.35 | 0.25 | 0.07 | 0.07 |
| 3066770 3005357 | | | | | | | | | | |
| knn: 2458376 2478155 | 0.77 | 0.049 | 0.262 | 0.076 | 0.50 | 0.75 | 0.46 | 0.25 | 0.09 | 0.08 |
| 3066770 3005357 | | | | | | | | | | |
| knn: 2458376 2478155 | 0.81 | 0.026 | 0.102 | 0.009 | 0.67 | 0.79 | 0.50 | 0.04 | 0.02 | 0.03 |
| 3066770 2651515 | | | | | | | | | | |
| rpart: 2458376 3139092 | 0.77 | 0.047 | 0.050 | 0.122 | 0.45 | 0.78 | 0.52 | 0.31 | 0.15 | 0.13 |
| 2512364 2364325 | | | | | | | | | | |
| nb: 2458376 3139092 | 0.78 | 0.046 | 0.137 | 0.058 | 0.50 | 0.80 | 0.37 | 0.13 | 0.07 | 0.07 |
| 2512364 3066770 | | | | | | | | | | |
| nb: 2458376 3139092 | 0.78 | 0.046 | 0.073 | 0.028 | 0.56 | 0.82 | 0.39 | 0.06 | 0.03 | 0.05 |
| 2512364 2651515 | | | | | | | | | | |
| rf: 2458376 2789391 | 0.84 | 0.014 | 0.017 | 0.022 | 0.67 | 0.91 | 0.53 | 0.01 | 0.03 | 0.05 |
| 2512364 2364325 | | | | | | | | | | |
| svm: 2458376 2789391 | 0.79 | 0.037 | 0.036 | 0.026 | 0.67 | 0.91 | 0.45 | 0.01 | 0.01 | 0.07 |
| 2512364 2364325 | | | | | | | | | | |
| rf: 2458376 2789391 | 0.81 | 0.024 | 0.005 | 0.022 | 0.67 | 0.91 | 0.52 | 0.01 | 0.04 | 0.05 |
| 2512364 3066770 | | | | | | | | | | |
| knn: 2458376 2789391 | 0.77 | 0.050 | 0.262 | 0.067 | 0.50 | 0.75 | 0.49 | 0.25 | 0.09 | 0.08 |
| 2512364 3005357 | | | | | | | | | | |
| rf: 2458376 2789391 | 0.84 | 0.017 | 0.022 | 0.031 | 0.60 | 0.90 | 0.52 | 0.02 | 0.03 | 0.06 |
| 2512364 2651515 | | | | | | | | | | |
| knn: 2458376 2789391 | 0.81 | 0.010 | 0.056 | 0.005 | 0.67 | 0.91 | 0.50 | 0.01 | 0.03 | 0.02 |
| 3066770 2651515 | | | | | | | | | | |
| rf: 2458376 3463598 | 0.81 | 0.024 | 0.007 | 0.032 | 0.46 | 0.86 | 0.43 | 0.18 | 0.05 | 0.07 |
| 2461837 2512364 | | | | | | | | | | |
| rpart: 2458376 3463598 | 0.79 | 0.037 | 0.216 | 0.044 | 0.56 | 0.82 | 0.62 | 0.09 | 0.08 | 0.06 |
| 2461837 2512364 | | | | | | | | | | |
| svm: 2458376 3463598 | 0.80 | 0.030 | 0.111 | 0.030 | 0.44 | 0.73 | −0.20 | 0.44 | 0.05 | 0.05 |
| 2461837 3005357 | | | | | | | | | | |
| hdda: 2458376 3463598 | 0.78 | 0.046 | 0.137 | 0.034 | 0.75 | 0.75 | 0.53 | 0.06 | 0.05 | 0.06 |
| 2512364 2364325 | | | | | | | | | | |
| rf: 2458376 3463598 | 0.80 | 0.030 | 0.094 | 0.018 | 0.60 | 0.90 | 0.52 | 0.01 | 0.02 | 0.06 |
| 2512364 2364325 | | | | | | | | | | |
| knn: 2458376 3463598 | 0.77 | 0.043 | 0.475 | 0.035 | 0.50 | 0.88 | 0.50 | 0.16 | 0.05 | 0.06 |
| 2512364 2364325 | | | | | | | | | | |
| svm: 2458376 3463598 | 0.82 | 0.019 | 0.012 | 0.024 | 0.60 | 0.73 | 0.06 | 0.21 | 0.04 | 0.05 |
| 2512364 2364325 | | | | | | | | | | |
| rf: 2458376 3463598 | 0.81 | 0.026 | 0.050 | 0.020 | 0.45 | 0.78 | 0.44 | 0.22 | 0.02 | 0.08 |
| 2512364 3066770 | | | | | | | | | | |
| svm: 2458376 3463598 | 0.88 | 0.005 | 0.005 | 0.003 | 0.64 | 1.00 | −0.45 | 0.00 | 0.01 | 0.03 |
| 2512364 3066770 | | | | | | | | | | |
| knn: 2458376 3463598 | 0.85 | 0.013 | 0.026 | 0.011 | 0.56 | 0.82 | 0.50 | 0.07 | 0.03 | 0.03 |
| 2512364 3005357 | | | | | | | | | | |
| rf: 2458376 3463598 | 0.78 | 0.047 | 0.056 | 0.061 | 0.67 | 0.91 | 0.43 | 0.01 | 0.04 | 0.08 |
| 2512364 2651515 | | | | | | | | | | |
| rf: 2458376 2461837 | 0.78 | 0.047 | 0.216 | 0.040 | 0.55 | 0.89 | 0.50 | 0.06 | 0.07 | 0.06 |
| 2512364 2364325 | | | | | | | | | | |
| knn: 2458376 2461837 | 0.80 | 0.033 | 0.287 | 0.109 | 0.50 | 0.75 | 0.52 | 0.25 | 0.12 | 0.11 |
| 2512364 2956442 | | | | | | | | | | |
| knn: 2458376 2461837 | 0.79 | 0.039 | 0.102 | 0.039 | 0.55 | 0.89 | 0.49 | 0.06 | 0.07 | 0.06 |
| 2512364 3005357 | | | | | | | | | | |
| rf: 2458376 2461837 | 0.78 | 0.047 | 0.115 | 0.031 | 0.71 | 0.85 | 0.60 | 0.02 | 0.07 | 0.05 |
| 2512364 2651515 | | | | | | | | | | |
| nb: 2458376 2461837 | 0.78 | 0.046 | 0.073 | 0.022 | 0.55 | 0.89 | 0.34 | 0.06 | 0.04 | 0.05 |
| 2512364 2651515 | | | | | | | | | | |
| knn: 2458376 2461837 | 0.79 | 0.035 | 0.128 | 0.096 | 0.50 | 0.88 | 0.45 | 0.11 | 0.10 | 0.12 |
| 2512364 2651515 | | | | | | | | | | |
| rf: 2458376 2512364 | 0.85 | 0.013 | 0.022 | 0.007 | 0.67 | 0.91 | 0.52 | 0.01 | 0.01 | 0.03 |
| 2364325 3066770 | | | | | | | | | | |
| nb: 2458376 2512364 | 0.82 | 0.019 | 0.046 | 0.041 | 0.63 | 0.83 | 0.45 | 0.03 | 0.05 | 0.05 |
| 2364325 3066770 | | | | | | | | | | |
| svm: 2458376 2512364 | 0.84 | 0.014 | 0.025 | 0.004 | 0.83 | 0.86 | 0.75 | 0.00 | 0.01 | 0.02 |
| 2364325 3066770 | | | | | | | | | | |
| rf: 2458376 2512364 | 0.84 | 0.014 | 0.073 | 0.024 | 0.50 | 0.88 | 0.49 | 0.07 | 0.02 | 0.05 |
| 2364325 3005357 | | | | | | | | | | |
| nb: 2458376 2512364 | 0.80 | 0.030 | 0.169 | 0.042 | 0.56 | 0.82 | 0.43 | 0.07 | 0.06 | 0.06 |
| 2364325 3005357 | | | | | | | | | | |
| knn: 2458376 2512364 | 0.79 | 0.035 | 0.511 | 0.015 | 0.57 | 0.77 | 0.64 | 0.08 | 0.01 | 0.06 |
| 2364325 3005357 | | | | | | | | | | |
| svm: 2458376 2512364 | 0.79 | 0.037 | 0.073 | 0.037 | 0.56 | 0.82 | 0.39 | 0.07 | 0.06 | 0.06 |
| 2364325 3005357 | | | | | | | | | | |
| nb: 2458376 2512364 | 0.78 | 0.046 | 0.169 | 0.029 | 0.63 | 0.83 | 0.40 | 0.03 | 0.03 | 0.04 |
| 2364325 2651515 | | | | | | | | | | |
| nb: 2458376 2512364 | 0.79 | 0.037 | 0.137 | 0.039 | 0.56 | 0.82 | 0.36 | 0.06 | 0.06 | 0.05 |
| 3066770 3005357 | | | | | | | | | | |
| svm: 2458376 2512364 | 0.79 | 0.037 | 0.137 | 0.028 | 0.56 | 0.82 | −0.22 | 0.06 | 0.04 | 0.05 |
| 3066770 3005357 | | | | | | | | | | |
| rf: 2458376 2512364 | 0.89 | 0.003 | 0.003 | 0.010 | 0.75 | 0.75 | 0.66 | 0.03 | 0.02 | 0.03 |
| 3066770 2651515 | | | | | | | | | | |
| nb: 2458376 2512364 | 0.82 | 0.019 | 0.073 | 0.019 | 0.56 | 0.82 | 0.34 | 0.06 | 0.02 | 0.04 |
| 3066770 2651515 | | | | | | | | | | |
| rf: 2458376 2512364 | 0.78 | 0.046 | 0.137 | 0.030 | 0.55 | 0.89 | 0.47 | 0.03 | 0.04 | 0.05 |
| 2956442 3005357 | | | | | | | | | | |
| rf: 2458376 2512364 | 0.84 | 0.014 | 0.017 | 0.060 | 0.57 | 0.77 | 0.64 | 0.07 | 0.04 | 0.07 |
| 3005357 2651515 | | | | | | | | | | |
| nb: 2458376 2512364 | 0.80 | 0.030 | 0.054 | 0.023 | 0.63 | 0.83 | 0.38 | 0.03 | 0.03 | 0.04 |
| 3005357 2651515 | | | | | | | | | | |
| nb: 2458376 2364325 | 0.78 | 0.046 | 0.094 | 0.052 | 0.57 | 0.77 | 0.41 | 0.08 | 0.04 | 0.06 |
| 3066770 2651515 | | | | | | | | | | |
| hdda: 3414753 2478155 | 0.78 | 0.046 | 0.111 | 0.122 | 0.44 | 0.73 | 0.48 | 0.39 | 0.13 | 0.13 |
| 3463598 2512364 | | | | | | | | | | |
| rpart: 3414753 2478155 | 0.80 | 0.030 | 0.050 | 0.003 | 0.83 | 0.86 | 0.61 | 0.00 | 0.01 | 0.01 |
| 3463598 2512364 | | | | | | | | | | |
| svm: 3414753 2478155 | 0.78 | 0.046 | 0.111 | 0.079 | 0.56 | 0.82 | −0.07 | 0.06 | 0.07 | 0.09 |
| 2512364 2364325 | | | | | | | | | | |
| rf: 3414753 2478155 | 0.80 | 0.036 | 0.143 | 0.056 | 0.50 | 0.80 | 0.44 | 0.11 | 0.06 | 0.07 |
| 2512364 3066770 | | | | | | | | | | |
| knn: 3414753 2478155 | 0.78 | 0.046 | 0.064 | 0.102 | 0.50 | 0.75 | 0.54 | 0.25 | 0.10 | 0.11 |
| 2512364 3066770 | | | | | | | | | | |
| svm: 3414753 2478155 | 0.81 | 0.024 | 0.111 | 0.022 | 0.57 | 0.77 | 0.78 | 0.09 | 0.02 | 0.04 |
| 2512364 3066770 | | | | | | | | | | |
| svm: 3414753 2478155 | 0.78 | 0.046 | 0.073 | 0.052 | 0.63 | 0.83 | −0.09 | 0.03 | 0.06 | 0.07 |
| 3066770 2651515 | | | | | | | | | | |
| rpart: 3414753 2478155 | 0.79 | 0.032 | 0.262 | 0.021 | 0.50 | 1.00 | 0.46 | 0.04 | 0.04 | 0.07 |
| 2956442 3005357 | | | | | | | | | | |
| rpart: 3414753 3139092 | 0.78 | 0.043 | 0.216 | 0.069 | 0.55 | 0.89 | 0.59 | 0.06 | 0.11 | 0.09 |
| 3463598 2512364 | | | | | | | | | | |
| rpart: 3414753 3139092 | 0.78 | 0.043 | 0.216 | 0.069 | 0.55 | 0.89 | 0.59 | 0.06 | 0.11 | 0.09 |
| 2512364 3066770 | | | | | | | | | | |
| rpart: 3414753 3139092 | 0.78 | 0.043 | 0.216 | 0.069 | 0.55 | 0.89 | 0.59 | 0.06 | 0.11 | 0.09 |
| 2512364 3005357 | | | | | | | | | | |
| svm: 3414753 2789391 | 0.86 | 0.008 | 0.025 | 0.002 | 0.71 | 0.85 | 0.63 | 0.01 | 0.01 | 0.02 |
| 2461837 2512364 | | | | | | | | | | |
| knn: 3414753 3463598 | 0.77 | 0.049 | 0.262 | 0.041 | 0.55 | 0.89 | 0.50 | 0.05 | 0.08 | 0.06 |
| 2461837 2651515 | | | | | | | | | | |
| rpart: 3414753 2461837 | 0.79 | 0.029 | 0.115 | 0.013 | 0.71 | 0.85 | 0.63 | 0.03 | 0.05 | 0.02 |
| 2512364 3066770 | | | | | | | | | | |
| rf: 3414753 2461837 | 0.83 | 0.019 | 0.064 | 0.058 | 0.50 | 0.75 | 0.54 | 0.25 | 0.07 | 0.07 |
| 2512364 3005357 | | | | | | | | | | |
| rpart: 3414753 2461837 | 0.79 | 0.029 | 0.115 | 0.013 | 0.71 | 0.85 | 0.63 | 0.03 | 0.05 | 0.02 |
| 2512364 3005357 | | | | | | | | | | |
| svm: 3414753 2512364 | 0.78 | 0.046 | 0.094 | 0.082 | 0.56 | 0.82 | −0.17 | 0.06 | 0.08 | 0.09 |
| 2364325 3066770 | | | | | | | | | | |
| svm: 3414753 2512364 | 0.84 | 0.014 | 0.046 | 0.073 | 0.56 | 0.82 | 0.00 | 0.06 | 0.08 | 0.08 |
| 3066770 3005357 | | | | | | | | | | |
| rf: 3414753 2512364 | 0.79 | 0.037 | 0.169 | 0.044 | 0.63 | 0.83 | 0.55 | 0.02 | 0.05 | 0.06 |
| 2956442 3005357 | | | | | | | | | | |
| rpart: 3414753 2512364 | 0.78 | 0.045 | 0.287 | 0.039 | 0.56 | 0.82 | 0.61 | 0.10 | 0.06 | 0.06 |
| 2956442 2651515 | | | | | | | | | | |
| svm: 3414753 2512364 | 0.78 | 0.046 | 0.073 | 0.019 | 0.71 | 0.85 | 0.09 | 0.01 | 0.02 | 0.04 |
| 3005357 2651515 | | | | | | | | | | |
| rpart: 2332285 2478155 | 0.81 | 0.027 | 0.216 | 0.061 | 0.57 | 0.77 | 0.43 | 0.15 | 0.08 | 0.07 |
| 2789391 3463598 | | | | | | | | | | |
| rf: 2332285 2478155 | 0.84 | 0.017 | 0.050 | 0.016 | 0.80 | 0.80 | 0.52 | 0.02 | 0.02 | 0.05 |
| 3463598 2512364 | | | | | | | | | | |
| rf: 2332285 2478155 | 0.82 | 0.019 | 0.036 | 0.015 | 0.63 | 0.83 | 0.51 | 0.02 | 0.01 | 0.03 |
| 2512364 2364325 | | | | | | | | | | |
| rf: 2332285 2478155 | 0.81 | 0.026 | 0.064 | 0.030 | 0.56 | 0.82 | 0.46 | 0.06 | 0.03 | 0.06 |
| 2512364 3066770 | | | | | | | | | | |
| rf: 2332285 2478155 | 0.79 | 0.043 | 0.056 | 0.028 | 0.60 | 0.90 | 0.46 | 0.02 | 0.05 | 0.05 |
| 2512364 2956442 | | | | | | | | | | |
| rf: 2332285 2478155 | 0.79 | 0.043 | 0.128 | 0.030 | 0.60 | 0.90 | 0.49 | 0.01 | 0.04 | 0.05 |
| 2512364 3005357 | | | | | | | | | | |
| rf: 2332285 3463598 | 0.79 | 0.037 | 0.017 | 0.074 | 0.50 | 0.75 | 0.49 | 0.37 | 0.10 | 0.09 |
| 2956442 3005357 | | | | | | | | | | |
| knn: 2332285 3463598 | 0.82 | 0.016 | 0.238 | 0.009 | 0.63 | 0.83 | 0.50 | 0.01 | 0.01 | 0.03 |
| 3005357 2651515 | | | | | | | | | | |
| rpart: 2332285 2512364 | 0.80 | 0.025 | 0.128 | 0.013 | 0.60 | 0.90 | 0.59 | 0.02 | 0.04 | 0.03 |
| 2364325 3066770 | | | | | | | | | | |
| svm: 2332285 2512364 | 0.82 | 0.019 | 0.073 | 0.023 | 0.56 | 0.82 | 0.24 | 0.06 | 0.03 | 0.04 |
| 2364325 3066770 | | | | | | | | | | |
| rf: 2332285 2512364 | 0.80 | 0.030 | 0.046 | 0.030 | 0.55 | 0.89 | 0.46 | 0.06 | 0.04 | 0.05 |
| 2364325 2956442 | | | | | | | | | | |
| rf: 2332285 2512364 | 0.80 | 0.036 | 0.115 | 0.050 | 0.50 | 0.80 | 0.51 | 0.13 | 0.05 | 0.07 |
| 2364325 3005357 | | | | | | | | | | |
| knn: 2332285 2512364 | 0.77 | 0.048 | 0.439 | 0.117 | 0.50 | 0.75 | 0.51 | 0.25 | 0.13 | 0.12 |
| 2956442 3005357 | | | | | | | | | | |
| rpart: 2478155 3139092 | 0.84 | 0.014 | 0.050 | 0.029 | 0.55 | 0.89 | 0.54 | 0.07 | 0.08 | 0.06 |
| 3463598 2512364 | | | | | | | | | | |
| svm: 2478155 3139092 | 0.84 | 0.014 | 0.007 | 0.053 | 0.35 | 0.67 | −0.76 | 0.98 | 0.04 | 0.08 |
| 2461837 2651515 | | | | | | | | | | |
| rpart: 2478155 3139092 | 0.84 | 0.014 | 0.050 | 0.046 | 0.55 | 0.89 | 0.62 | 0.06 | 0.10 | 0.07 |
| 2512364 3066770 | | | | | | | | | | |
| rf: 2478155 3139092 | 0.78 | 0.047 | 0.022 | 0.102 | 0.40 | 0.80 | 0.33 | 0.46 | 0.12 | 0.12 |
| 2512364 2956442 | | | | | | | | | | |
| rpart: 2478155 3139092 | 0.77 | 0.049 | 0.216 | 0.063 | 0.50 | 0.88 | 0.46 | 0.11 | 0.09 | 0.08 |
| 2512364 2956442 | | | | | | | | | | |
| rpart: 2478155 2789391 | 0.79 | 0.040 | 0.216 | 0.035 | 0.67 | 0.79 | 0.48 | 0.06 | 0.05 | 0.05 |
| 3463598 2512364 | | | | | | | | | | |
| rf: 2478155 2789391 | 0.78 | 0.046 | 0.073 | 0.048 | 0.55 | 0.89 | 0.43 | 0.06 | 0.09 | 0.06 |
| 2461837 2512364 | | | | | | | | | | |
| svm: 2478155 2789391 | 0.78 | 0.046 | 0.073 | 0.028 | 0.71 | 0.85 | 0.47 | 0.01 | 0.01 | 0.08 |
| 2461837 2512364 | | | | | | | | | | |
| rf: 2478155 2789391 | 0.82 | 0.019 | 0.017 | 0.018 | 0.67 | 0.91 | 0.49 | 0.01 | 0.03 | 0.04 |
| 2512364 2364325 | | | | | | | | | | |
| rf: 2478155 2789391 | 0.79 | 0.037 | 0.036 | 0.030 | 0.67 | 0.91 | 0.42 | 0.01 | 0.05 | 0.05 |
| 2512364 3066770 | | | | | | | | | | |
| rf: 2478155 3463598 | 0.82 | 0.024 | 0.056 | 0.028 | 0.46 | 0.86 | 0.42 | 0.18 | 0.06 | 0.05 |
| 2461837 2512364 | | | | | | | | | | |
| rpart: 2478155 3463598 | 0.79 | 0.036 | 0.115 | 0.016 | 0.71 | 0.85 | 0.54 | 0.01 | 0.05 | 0.03 |
| 2461837 2512364 | | | | | | | | | | |
| rf: 2478155 3463598 | 0.84 | 0.014 | 0.046 | 0.010 | 0.50 | 0.71 | 0.55 | 0.25 | 0.01 | 0.04 |
| 2512364 2364325 | | | | | | | | | | |
| rpart: 2478155 3463598 | 0.82 | 0.019 | 0.050 | 0.002 | 0.83 | 0.86 | 0.62 | 0.00 | 0.01 | 0.01 |
| 2512364 2364325 | | | | | | | | | | |
| rf: 2478155 3463598 | 0.80 | 0.030 | 0.137 | 0.022 | 0.56 | 0.82 | 0.41 | 0.09 | 0.02 | 0.07 |
| 2512364 3066770 | | | | | | | | | | |
| rf: 2478155 3463598 | 0.81 | 0.026 | 0.022 | 0.012 | 0.60 | 0.90 | 0.44 | 0.03 | 0.03 | 0.03 |
| 2512364 2956442 | | | | | | | | | | |
| rf: 2478155 3463598 | 0.79 | 0.043 | 0.143 | 0.012 | 0.67 | 0.79 | 0.49 | 0.06 | 0.01 | 0.05 |
| 2512364 3005357 | | | | | | | | | | |
| rf: 2478155 2461837 | 0.78 | 0.046 | 0.137 | 0.037 | 0.43 | 0.83 | 0.33 | 0.30 | 0.07 | 0.06 |
| 2512364 2364325 | | | | | | | | | | |
| rf: 2478155 2461837 | 0.81 | 0.026 | 0.056 | 0.021 | 0.55 | 0.89 | 0.43 | 0.05 | 0.04 | 0.04 |
| 2512364 2956442 | | | | | | | | | | |
| rf: 2478155 2512364 | 0.85 | 0.014 | 0.022 | 0.006 | 0.67 | 0.91 | 0.46 | 0.01 | 0.01 | 0.03 |
| 2364325 3066770 | | | | | | | | | | |
| svm: 2478155 2512364 | 0.78 | 0.046 | 0.111 | 0.039 | 0.44 | 0.73 | 0.05 | 0.47 | 0.05 | 0.06 |
| 2364325 3066770 | | | | | | | | | | |
| rf: 2478155 2512364 | 0.84 | 0.014 | 0.012 | 0.006 | 0.67 | 0.91 | 0.49 | 0.01 | 0.01 | 0.02 |
| 2364325 2956442 | | | | | | | | | | |
| rf: 2478155 2512364 | 0.81 | 0.024 | 0.046 | 0.040 | 0.60 | 0.90 | 0.44 | 0.02 | 0.02 | 0.06 |
| 2364325 2651515 | | | | | | | | | | |
| rf: 2478155 2512364 | 0.85 | 0.011 | 0.036 | 0.013 | 0.50 | 0.88 | 0.38 | 0.10 | 0.03 | 0.04 |
| 3066770 2956442 | | | | | | | | | | |
| rf: 2478155 2512364 | 0.81 | 0.024 | 0.036 | 0.014 | 0.63 | 0.83 | 0.54 | 0.02 | 0.02 | 0.03 |
| 3066770 3005357 | | | | | | | | | | |
| svm: 2478155 2512364 | 0.78 | 0.046 | 0.111 | 0.037 | 0.56 | 0.82 | 0.09 | 0.06 | 0.04 | 0.06 |
| 3066770 3005357 | | | | | | | | | | |
| rf: 2478155 2512364 | 0.78 | 0.047 | 0.115 | 0.088 | 0.60 | 0.90 | 0.33 | 0.02 | 0.04 | 0.11 |
| 3066770 2651515 | | | | | | | | | | |
| svm: 2478155 2512364 | 0.80 | 0.030 | 0.017 | 0.043 | 0.60 | 0.73 | −0.01 | 0.07 | 0.02 | 0.06 |
| 3066770 2651515 | | | | | | | | | | |
| rf: 2478155 2512364 | 0.82 | 0.024 | 0.056 | 0.008 | 0.58 | 1.00 | 0.43 | 0.01 | 0.02 | 0.03 |
| 2956442 3005357 | | | | | | | | | | |
| rpart: 2478155 2512364 | 0.77 | 0.036 | 0.262 | 0.028 | 0.50 | 1.00 | 0.44 | 0.04 | 0.16 | 0.17 |
| 2956442 3005357 | | | | | | | | | | |
| rf: 2478155 2512364 | 0.80 | 0.032 | 0.128 | 0.030 | 0.55 | 0.89 | 0.45 | 0.05 | 0.04 | 0.05 |
| 2956442 2651515 | | | | | | | | | | |
| rpart: 2478155 2512364 | 0.79 | 0.034 | 0.128 | 0.015 | 0.60 | 0.90 | 0.62 | 0.02 | 0.05 | 0.04 |
| 2956442 2651515 | | | | | | | | | | |
| svm: 2478155 2512364 | 0.80 | 0.030 | 0.094 | 0.029 | 0.56 | 0.82 | −0.08 | 0.06 | 0.03 | 0.04 |
| 3005357 2651515 | | | | | | | | | | |
| rpart: 3139092 2789391 | 0.78 | 0.044 | 0.050 | 0.184 | 0.46 | 0.86 | 0.53 | 0.22 | 0.24 | 0.19 |
| 3463598 2512364 | | | | | | | | | | |
| knn: 3139092 2789391 | 0.81 | 0.021 | 0.050 | 0.009 | 0.83 | 0.86 | 0.50 | 0.00 | 0.01 | 0.03 |
| 2461837 2651515 | | | | | | | | | | |
| svm: 3139092 3463598 | 0.79 | 0.037 | 0.036 | 0.116 | 0.40 | 0.80 | −0.38 | 0.46 | 0.14 | 0.14 |
| 2461837 3005357 | | | | | | | | | | |
| knn: 3139092 3463598 | 0.77 | 0.025 | 0.128 | 0.018 | 0.60 | 0.90 | 0.50 | 0.03 | 0.06 | 0.04 |
| 2461837 2651515 | | | | | | | | | | |
| svm: 3139092 3463598 | 0.84 | 0.014 | 0.012 | 0.011 | 0.55 | 0.89 | −0.08 | 0.06 | 0.01 | 0.05 |
| 2461837 2651515 | | | | | | | | | | |
| rpart: 3139092 3463598 | 0.84 | 0.014 | 0.022 | 0.008 | 0.60 | 0.90 | 0.52 | 0.04 | 0.04 | 0.03 |
| 2512364 3066770 | | | | | | | | | | |
| rpart: 3139092 3463598 | 0.83 | 0.016 | 0.022 | 0.005 | 0.75 | 0.92 | 0.54 | 0.00 | 0.03 | 0.02 |
| 2512364 3005357 | | | | | | | | | | |
| knn: 3139092 3463598 | 0.74 | 0.045 | 0.238 | 0.036 | 0.63 | 0.83 | 0.50 | 0.05 | 0.08 | 0.05 |
| 3066770 2651515 | | | | | | | | | | |
| knn: 3139092 2512364 | 0.80 | 0.034 | 0.216 | 0.029 | 0.67 | 0.79 | 0.59 | 0.05 | 0.04 | 0.05 |
| 3066770 3005357 | | | | | | | | | | |
| rpart: 3139092 2512364 | 0.78 | 0.044 | 0.050 | 0.037 | 0.63 | 0.83 | 0.56 | 0.03 | 0.05 | 0.05 |
| 2956442 3005357 | | | | | | | | | | |
| svm: 3139092 2512364 | 0.78 | 0.046 | 0.257 | 0.056 | 0.14 | 0.54 | −0.18 | 0.18 | 0.07 | 0.08 |
| 2956442 2651515 | | | | | | | | | | |
| rf: 2789391 2461837 | 0.79 | 0.037 | 0.046 | 0.030 | 0.50 | 0.80 | 0.50 | 0.24 | 0.06 | 0.05 |
| 2512364 2364325 | | | | | | | | | | |
| rf: 2789391 2461837 | 0.79 | 0.043 | 0.128 | 0.032 | 0.55 | 0.89 | 0.44 | 0.06 | 0.08 | 0.05 |
| 2512364 2651515 | | | | | | | | | | |
| rf: 2789391 2512364 | 0.81 | 0.029 | 0.026 | 0.093 | 0.50 | 0.71 | 0.60 | 0.28 | 0.09 | 0.10 |
| 2364325 3066770 | | | | | | | | | | |
| rf: 2789391 2512364 | 0.82 | 0.022 | 0.026 | 0.042 | 0.50 | 0.71 | 0.61 | 0.28 | 0.05 | 0.06 |
| 2364325 2956442 | | | | | | | | | | |
| rf: 3463598 2461837 | 0.81 | 0.024 | 0.046 | 0.020 | 0.56 | 0.82 | 0.48 | 0.15 | 0.03 | 0.05 |
| 2512364 2364325 | | | | | | | | | | |
| rf: 3463598 2461837 | 0.79 | 0.037 | 0.073 | 0.015 | 0.63 | 0.83 | 0.49 | 0.07 | 0.04 | 0.03 |
| 2512364 2956442 | | | | | | | | | | |
| rf: 3463598 2512364 | 0.87 | 0.009 | 0.026 | 0.013 | 0.67 | 0.71 | 0.75 | 0.20 | 0.02 | 0.05 |
| 2364325 2956442 | | | | | | | | | | |
| rf: 3463598 2512364 | 0.78 | 0.047 | 0.056 | 0.030 | 0.50 | 0.88 | 0.34 | 0.07 | 0.02 | 0.06 |
| 3066770 3005357 | | | | | | | | | | |
| rf: 3463598 2512364 | 0.85 | 0.011 | 0.017 | 0.006 | 0.58 | 1.00 | 0.41 | 0.01 | 0.02 | 0.03 |
| 2956442 3005357 | | | | | | | | | | |
| knn: 3463598 2956442 | 0.78 | 0.046 | 0.238 | 0.086 | 0.50 | 0.71 | 0.53 | 0.33 | 0.08 | 0.09 |
| 3005357 2651515 | | | | | | | | | | |
| rf: 2461837 2512364 | 0.78 | 0.046 | 0.203 | 0.046 | 0.67 | 0.79 | 0.60 | 0.05 | 0.06 | 0.06 |
| 2364325 3066770 | | | | | | | | | | |
| rf: 2461837 2512364 | 0.78 | 0.047 | 0.128 | 0.043 | 0.50 | 0.75 | 0.55 | 0.25 | 0.06 | 0.06 |
| 2364325 2956442 | | | | | | | | | | |
| rf: 2461837 2512364 | 0.78 | 0.046 | 0.025 | 0.037 | 0.55 | 0.89 | 0.42 | 0.06 | 0.05 | 0.06 |
| 2364325 2651515 | | | | | | | | | | |
| rf: 2461837 2512364 | 0.79 | 0.037 | 0.094 | 0.038 | 0.50 | 0.75 | 0.55 | 0.25 | 0.06 | 0.05 |
| 2956442 3005357 | | | | | | | | | | |
| rf: 2461837 2512364 | 0.80 | 0.030 | 0.005 | 0.079 | 0.67 | 0.91 | 0.42 | 0.01 | 0.10 | 0.09 |
| 3005357 2651515 | | | | | | | | | | |
| svm: 2512364 2364325 | 0.78 | 0.046 | 0.036 | 0.074 | 0.71 | 0.85 | 0.89 | 0.01 | 0.09 | 0.09 |
| 3066770 2956442 | | | | | | | | | | |
| svm: 2512364 2364325 | 0.81 | 0.024 | 0.036 | 0.022 | 0.71 | 0.85 | 0.65 | 0.01 | 0.02 | 0.04 |
| 3066770 3005357 | | | | | | | | | | |
| rf: 2512364 3066770 | 0.80 | 0.030 | 0.036 | 0.026 | 0.63 | 0.83 | 0.59 | 0.02 | 0.03 | 0.04 |
| 2956442 3005357 | | | | | | | | | | |
| rf: 2512364 2956442 | 0.82 | 0.024 | 0.026 | 0.030 | 0.58 | 1.00 | 0.36 | 0.01 | 0.03 | 0.05 |
| 3005357 2651515 | | | | | | | | | | |
| knn: 2512364 2956442 | 0.77 | 0.043 | 0.439 | 0.030 | 0.56 | 0.82 | 0.50 | 0.07 | 0.05 | 0.06 |
| 3005357 2651515 | | | | | | | | | | |
| rpart: 2512364 2956442 | 0.80 | 0.031 | 0.143 | 0.019 | 0.63 | 0.83 | 0.56 | 0.03 | 0.03 | 0.03 |
| 3005357 2651515 | | | | | | | | | | |
| svm: 2512364 2956442 | 0.82 | 0.019 | 0.046 | 0.032 | 0.71 | 0.85 | 0.13 | 0.01 | 0.05 | 0.05 |
| 3005357 2651515 | | | | | | | | | | |

Performance of pairwise, threewise and fourwise combinations of the 15 markers across different metrics for the Recurrence endpoint for pT2 patients (organ-confined disease). All classifiers have a Wilcox P-value <=0.05

TABLE 14

| | | | | | | | | | UVA | UVA |
|---|---|---|---|---|---|---|---|---|---|---|
| | | WILCOX | KS | T-TEST | | | | KM | HR | OR |
| | | P- | P- | P- | | | | P- | P- | P- |
| CLASSIFIER | AUC | VALUE | VALUE | VALUE | PPV | NPV | CUTOFF | VALUE | VALUE | VALUE |
| knn: 2458376 | 0.80 | 0.022 | 0.137 | 0.030 | 0.83 | 0.38 | 0.53 | 0.32 | 0.11 | 0.05 |
| 2364325 | | | | | | | | | | |
| rf: 2458376 | 0.77 | 0.046 | 0.051 | 0.026 | 0.86 | 0.45 | 0.52 | 0.21 | 0.15 | 0.04 |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 3005357 | | | | | | | | | | |
| rpart: 3139092 | 0.77 | 0.011 | 0.097 | 0.007 | 0.88 | 0.63 | 0.44 | 0.02 | 0.03 | 0.02 |
| 3005357 | | | | | | | | | | |
| knn: 2512364 | 0.77 | 0.040 | 0.097 | 0.066 | 0.92 | 0.50 | 0.50 | 0.05 | 0.11 | 0.08 |
| 3005357 | | | | | | | | | | |
| rpart: 2512364 | 0.79 | 0.026 | 0.068 | 0.012 | 0.91 | 0.43 | 0.62 | 0.11 | 0.03 | 0.03 |
| 3005357 | | | | | | | | | | |
| rpart: 2364325 | 0.77 | 0.038 | 0.097 | 0.032 | 0.88 | 0.63 | 0.37 | 0.02 | 0.07 | 0.05 |
| 3005357 | | | | | | | | | | |
| rpart: 3066770 | 0.81 | 0.010 | 0.097 | 0.006 | 0.92 | 0.50 | 0.56 | 0.03 | 0.02 | 0.02 |
| 3005357 | | | | | | | | | | |
| rpart: 2956442 | 0.77 | 0.011 | 0.097 | 0.007 | 0.88 | 0.63 | 0.44 | 0.02 | 0.03 | 0.02 |
| 3005357 | | | | | | | | | | |
| rf: 3005357 | 0.80 | 0.023 | 0.016 | 0.015 | 0.92 | 0.50 | 0.45 | 0.05 | 0.09 | 0.03 |
| 2651515 | | | | | | | | | | |
| knn: 3005357 | 0.76 | 0.045 | 0.137 | 0.062 | 0.89 | 0.38 | 0.50 | 0.53 | 0.37 | 0.08 |
| 2651515 | | | | | | | | | | |
| knn: 3326487 | 0.75 | 0.046 | 0.292 | 0.029 | 0.87 | 0.50 | 0.50 | 0.20 | 0.13 | 0.05 |
| 2332285 2512364 | | | | | | | | | | |
| rpart: 3326487 | 0.75 | 0.049 | 0.097 | 0.093 | 0.88 | 0.35 | 0.38 | 0.08 | 0.03 | 0.12 |
| 2364325 3005357 | | | | | | | | | | |
| rpart: 3326487 | 0.77 | 0.011 | 0.097 | 0.007 | 0.88 | 0.63 | 0.44 | 0.02 | 0.03 | 0.02 |
| 3066770 3005357 | | | | | | | | | | |
| rpart: 3326487 | 0.85 | 0.007 | 0.025 | 0.002 | 0.93 | 0.60 | 0.48 | 0.02 | 0.04 | 0.01 |
| 3005357 2651515 | | | | | | | | | | |
| svm: 2458376 | 0.78 | 0.034 | 0.043 | 0.103 | 0.84 | 0.67 | −0.28 | 0.09 | 0.19 | 0.11 |
| 2332285 3139092 | | | | | | | | | | |
| knn: 2458376 | 0.79 | 0.028 | 0.126 | 0.035 | 0.81 | 0.44 | 0.50 | 0.45 | 0.20 | 0.05 |
| 2332285 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 2332285 3005357 | | | | | | | | | | |
| svm: 2458376 | 0.76 | 0.047 | 0.046 | 0.021 | 0.50 | 0.13 | −0.59 | 0.05 | 0.13 | 0.07 |
| 2478155 2461837 | | | | | | | | | | |
| rpart: 2458376 | 0.77 | 0.011 | 0.097 | 0.007 | 0.88 | 0.63 | 0.44 | 0.02 | 0.03 | 0.02 |
| 2478155 3005357 | | | | | | | | | | |
| knn: 2458376 | 0.77 | 0.035 | 0.089 | 0.056 | 0.77 | 0.67 | 0.32 | 0.27 | 0.15 | 0.08 |
| 3139092 3463598 | | | | | | | | | | |
| knn: 2458376 | 0.78 | 0.036 | 0.219 | 0.029 | 0.78 | 0.43 | 0.48 | 0.52 | 0.10 | 0.05 |
| 3139092 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3139092 3005357 | | | | | | | | | | |
| knn: 2458376 | 0.81 | 0.017 | 0.126 | 0.013 | 0.92 | 0.50 | 0.50 | 0.09 | 0.08 | 0.03 |
| 2512364 2956442 | | | | | | | | | | |
| rf: 2458376 | 0.80 | 0.023 | 0.016 | 0.032 | 0.83 | 0.32 | 0.70 | 0.72 | 0.14 | 0.05 |
| 2512364 3005357 | | | | | | | | | | |
| knn: 2458376 | 0.80 | 0.023 | 0.236 | 0.016 | 0.90 | 0.40 | 0.61 | 0.63 | 0.17 | 0.04 |
| 2512364 3005357 | | | | | | | | | | |
| svm: 2458376 | 0.77 | 0.041 | 0.043 | 0.028 | 0.87 | 0.50 | −0.05 | 0.19 | 0.17 | 0.04 |
| 2512364 3005357 | | | | | | | | | | |
| rf: 2458376 | 0.76 | 0.047 | 0.043 | 0.043 | 0.92 | 0.46 | 0.56 | 0.18 | 0.27 | 0.06 |
| 2512364 2651515 | | | | | | | | | | |
| knn: 2458376 | 0.81 | 0.021 | 0.097 | 0.010 | 0.86 | 0.45 | 0.51 | 0.16 | 0.08 | 0.02 |
| 2364325 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 2364325 3005357 | | | | | | | | | | |
| knn: 2458376 | 0.80 | 0.023 | 0.034 | 0.026 | 0.93 | 0.60 | 0.50 | 0.03 | 0.19 | 0.04 |
| 2364325 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 3066770 3005357 | | | | | | | | | | |
| svm: 2458376 | 0.80 | 0.021 | 0.043 | 0.019 | 0.92 | 0.46 | 1.16 | 0.37 | 0.15 | 0.04 |
| 3066770 2651515 | | | | | | | | | | |
| rf: 2458376 | 0.80 | 0.023 | 0.051 | 0.028 | 0.83 | 0.32 | 0.72 | 0.94 | 0.29 | 0.05 |
| 3005357 2651515 | | | | | | | | | | |
| rpart: 2458376 | 0.80 | 0.024 | 0.068 | 0.038 | 0.92 | 0.46 | 0.49 | 0.09 | 0.13 | 0.06 |
| 3005357 2651515 | | | | | | | | | | |
| svm: 3414753 | 0.78 | 0.034 | 0.008 | 0.105 | 0.70 | 0.20 | −0.16 | 0.57 | 0.09 | 0.12 |
| 2478155 2651515 | | | | | | | | | | |
| rf: 3414753 | 0.76 | 0.049 | 0.097 | 0.043 | 0.89 | 0.38 | 0.62 | 0.37 | 0.16 | 0.06 |
| 2512364 3005357 | | | | | | | | | | |
| rpart: 3414753 | 0.80 | 0.019 | 0.068 | 0.017 | 0.92 | 0.46 | 0.44 | 0.06 | 0.05 | 0.04 |
| 2512364 3005357 | | | | | | | | | | |
| knn: 3414753 | 0.77 | 0.035 | 0.074 | 0.023 | 0.85 | 0.42 | 0.50 | 0.42 | 0.16 | 0.04 |
| 2512364 2651515 | | | | | | | | | | |
| rpart: 3414753 | 0.77 | 0.011 | 0.097 | 0.007 | 0.88 | 0.63 | 0.44 | 0.02 | 0.03 | 0.02 |
| 2364325 3005357 | | | | | | | | | | |
| rpart: 3414753 | 0.77 | 0.011 | 0.097 | 0.007 | 0.88 | 0.63 | 0.44 | 0.02 | 0.03 | 0.02 |
| 2956442 3005357 | | | | | | | | | | |
| rf: 3414753 | 0.79 | 0.032 | 0.025 | 0.023 | 0.80 | 0.30 | 0.70 | 0.70 | 0.24 | 0.04 |
| 3005357 2651515 | | | | | | | | | | |
| svm: 2332285 | 0.76 | 0.047 | 0.020 | 0.039 | 0.93 | 0.55 | 0.16 | 0.05 | 0.06 | 0.06 |
| 3139092 2461837 | | | | | | | | | | |
| knn: 2332285 | 0.78 | 0.029 | 0.313 | 0.012 | 0.89 | 0.38 | 0.70 | 0.23 | 0.06 | 0.03 |
| 3139092 2512364 | | | | | | | | | | |
| rpart: 2332285 | 0.77 | 0.011 | 0.097 | 0.007 | 0.88 | 0.63 | 0.44 | 0.02 | 0.03 | 0.02 |
| 2789391 3005357 | | | | | | | | | | |
| knn: 2332285 | 0.76 | 0.048 | 0.292 | 0.040 | 0.87 | 0.50 | 0.55 | 0.19 | 0.26 | 0.05 |
| 2512364 2651515 | | | | | | | | | | |
| rpart: 2332285 | 0.77 | 0.011 | 0.097 | 0.007 | 0.88 | 0.63 | 0.44 | 0.02 | 0.03 | 0.02 |
| 3066770 3005357 | | | | | | | | | | |
| rpart: 2332285 | 0.85 | 0.007 | 0.025 | 0.002 | 0.93 | 0.60 | 0.48 | 0.02 | 0.04 | 0.01 |
| 3005357 2651515 | | | | | | | | | | |
| svm: 2478155 | 0.79 | 0.029 | 0.014 | 0.048 | 0.70 | 0.20 | −0.15 | 0.48 | 0.02 | 0.07 |
| 3139092 3005357 | | | | | | | | | | |
| svm: 2478155 | 0.76 | 0.047 | 0.068 | 0.040 | 0.91 | 0.43 | 0.45 | 0.17 | 0.10 | 0.06 |
| 2512364 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 2956442 3005357 | | | | | | | | | | |
| knn: 2478155 | 0.81 | 0.021 | 0.016 | 0.034 | 0.86 | 0.33 | 0.56 | 0.91 | 0.28 | 0.06 |
| 3005357 2651515 | | | | | | | | | | |
| rpart: 2478155 | 0.76 | 0.046 | 0.034 | 0.016 | 0.91 | 0.43 | 0.60 | 0.17 | 0.07 | 0.04 |
| 3005357 2651515 | | | | | | | | | | |
| knn: 3139092 | 0.82 | 0.013 | 0.089 | 0.010 | 0.83 | 0.57 | 0.50 | 0.10 | 0.01 | 0.02 |
| 3463598 2956442 | | | | | | | | | | |
| rpart: 3139092 | 0.77 | 0.011 | 0.097 | 0.007 | 0.88 | 0.63 | 0.44 | 0.02 | 0.03 | 0.02 |
| 2364325 3005357 | | | | | | | | | | |
| rpart: 3139092 | 0.77 | 0.011 | 0.097 | 0.007 | 0.88 | 0.63 | 0.44 | 0.02 | 0.03 | 0.02 |
| 3066770 3005357 | | | | | | | | | | |
| rpart: 3139092 | 0.77 | 0.011 | 0.097 | 0.007 | 0.88 | 0.63 | 0.44 | 0.02 | 0.03 | 0.02 |
| 2956442 3005357 | | | | | | | | | | |
| rpart: 2789391 | 0.77 | 0.011 | 0.097 | 0.007 | 0.88 | 0.63 | 0.44 | 0.02 | 0.03 | 0.02 |
| 3463598 3005357 | | | | | | | | | | |
| rpart: 2789391 | 0.76 | 0.027 | 0.068 | 0.008 | 0.93 | 0.55 | 0.48 | 0.01 | 0.01 | 0.02 |
| 2512364 3005357 | | | | | | | | | | |
| svm: 2789391 | 0.76 | 0.047 | 0.005 | 0.251 | 0.83 | 0.38 | −0.27 | 0.32 | 0.44 | 0.25 |
| 2512364 3005357 | | | | | | | | | | |
| rpart: 2789391 | 0.77 | 0.011 | 0.097 | 0.007 | 0.88 | 0.63 | 0.44 | 0.02 | 0.03 | 0.02 |
| 2956442 3005357 | | | | | | | | | | |
| rpart: 2789391 | 0.85 | 0.007 | 0.025 | 0.002 | 0.93 | 0.60 | 0.48 | 0.02 | 0.04 | 0.01 |
| 3005357 2651515 | | | | | | | | | | |
| rf: 3463598 | 0.77 | 0.046 | 0.051 | 0.037 | 0.89 | 0.38 | 0.67 | 0.22 | 0.12 | 0.06 |
| 2512364 3005357 | | | | | | | | | | |
| svm: 3463598 | 0.77 | 0.041 | 0.026 | 0.185 | 0.92 | 0.46 | −0.27 | 0.15 | 0.58 | 0.20 |
| 2956442 3005357 | | | | | | | | | | |
| rf: 3463598 | 0.80 | 0.021 | 0.014 | 0.044 | 0.89 | 0.38 | 0.61 | 0.50 | 0.28 | 0.07 |
| 3005357 2651515 | | | | | | | | | | |
| knn: 3463598 | 0.76 | 0.044 | 0.381 | 0.026 | 0.89 | 0.38 | 0.70 | 0.46 | 0.21 | 0.05 |
| 3005357 2651515 | | | | | | | | | | |
| svm: 2461837 | 0.77 | 0.041 | 0.130 | 0.030 | 0.80 | 0.60 | −3.54 | 0.16 | 0.13 | 0.05 |
| 2512364 3066770 | | | | | | | | | | |
| rf: 2461837 | 0.76 | 0.047 | 0.020 | 0.052 | 0.88 | 0.63 | 0.35 | 0.05 | 0.28 | 0.06 |
| 2512364 2651515 | | | | | | | | | | |
| svm: 2461837 | 0.78 | 0.034 | 0.111 | 0.296 | 0.76 | 0.50 | −4.24 | 0.31 | 0.21 | 0.31 |
| 3066770 3005357 | | | | | | | | | | |
| rf: 2461837 | 0.79 | 0.032 | 0.074 | 0.050 | 0.86 | 0.45 | 0.45 | 0.18 | 0.22 | 0.07 |
| 3005357 2651515 | | | | | | | | | | |
| rpart: 2512364 | 0.77 | 0.011 | 0.097 | 0.007 | 0.88 | 0.63 | 0.44 | 0.02 | 0.03 | 0.02 |
| 2364325 3005357 | | | | | | | | | | |
| rpart: 2512364 | 0.77 | 0.011 | 0.097 | 0.007 | 0.88 | 0.63 | 0.44 | 0.02 | 0.03 | 0.02 |
| 2956442 3005357 | | | | | | | | | | |
| rf: 2512364 | 0.81 | 0.018 | 0.003 | 0.017 | 0.93 | 0.60 | 0.37 | 0.02 | 0.18 | 0.04 |
| 3005357 2651515 | | | | | | | | | | |
| rpart: 2512364 | 0.85 | 0.007 | 0.025 | 0.002 | 0.93 | 0.60 | 0.48 | 0.02 | 0.04 | 0.01 |
| 3005357 2651515 | | | | | | | | | | |
| knn: 2364325 | 0.77 | 0.044 | 0.236 | 0.059 | 0.86 | 0.33 | 0.61 | 0.91 | 0.45 | 0.08 |
| 3005357 2651515 | | | | | | | | | | |
| rpart: 3066770 | 0.77 | 0.011 | 0.097 | 0.007 | 0.88 | 0.63 | 0.44 | 0.02 | 0.03 | 0.02 |
| 2956442 3005357 | | | | | | | | | | |
| rf: 3066770 | 0.79 | 0.032 | 0.025 | 0.075 | 0.88 | 0.35 | 0.59 | 0.73 | 0.38 | 0.11 |
| 3005357 2651515 | | | | | | | | | | |
| rf: 2956442 | 0.79 | 0.029 | 0.020 | 0.035 | 0.93 | 0.60 | 0.39 | 0.01 | 0.16 | 0.05 |
| 3005357 2651515 | | | | | | | | | | |
| rpart: 3326487 | 0.77 | 0.011 | 0.097 | 0.007 | 0.88 | 0.63 | 0.44 | 0.02 | 0.03 | 0.02 |
| 2458376 2364325 | | | | | | | | | | |
| 3005357 | | | | | | | | | | |
| rpart: 3326487 | 0.77 | 0.011 | 0.097 | 0.007 | 0.88 | 0.63 | 0.44 | 0.02 | 0.03 | 0.02 |
| 2458376 3066770 | | | | | | | | | | |
| 3005357 | | | | | | | | | | |
| rf: 3326487 | 0.76 | 0.049 | 0.074 | 0.067 | 0.88 | 0.35 | 0.64 | 0.50 | 0.31 | 0.09 |
| 2458376 3005357 | | | | | | | | | | |
| 2651515 | | | | | | | | | | |
| rpart: 3326487 | 0.77 | 0.011 | 0.097 | 0.007 | 0.88 | 0.63 | 0.44 | 0.02 | 0.03 | 0.02 |
| 3414753 3139092 | | | | | | | | | | |
| 3005357 | | | | | | | | | | |
| rpart: 3326487 | 0.82 | 0.007 | 0.068 | 0.004 | 0.93 | 0.55 | 0.55 | 0.01 | 0.01 | 0.02 |
| 3414753 2512364 | | | | | | | | | | |
| 3005357 | | | | | | | | | | |
| rpart: 3326487 | 0.77 | 0.011 | 0.097 | 0.007 | 0.88 | 0.63 | 0.44 | 0.02 | 0.03 | 0.02 |
| 3414753 3066770 | | | | | | | | | | |
| 3005357 | | | | | | | | | | |
| rpart: 3326487 | 0.77 | 0.011 | 0.097 | 0.007 | 0.88 | 0.63 | 0.44 | 0.02 | 0.03 | 0.02 |
| 3414753 2956442 | | | | | | | | | | |
| 3005357 | | | | | | | | | | |
| svm: 3326487 | 0.80 | 0.021 | 0.020 | 0.050 | 0.90 | 0.40 | 0.53 | 0.23 | 0.02 | 0.06 |
| 2332285 3139092 | | | | | | | | | | |
| 2512364 | | | | | | | | | | |
| rf: 3326487 | 0.79 | 0.029 | 0.025 | 0.117 | 0.88 | 0.35 | 0.59 | 0.73 | 0.45 | 0.14 |
| 2478155 3005357 | | | | | | | | | | |
| 2651515 | | | | | | | | | | |
| rpart: 3326487 | 0.77 | 0.011 | 0.097 | 0.007 | 0.88 | 0.63 | 0.44 | 0.02 | 0.03 | 0.02 |
| 3139092 3463598 | | | | | | | | | | |
| 3005357 | | | | | | | | | | |
| rpart: 3326487 | 0.82 | 0.007 | 0.068 | 0.004 | 0.93 | 0.55 | 0.55 | 0.01 | 0.01 | 0.02 |
| 2789391 2512364 | | | | | | | | | | |
| 3005357 | | | | | | | | | | |
| rpart: 3326487 | 0.77 | 0.011 | 0.097 | 0.007 | 0.88 | 0.63 | 0.44 | 0.02 | 0.03 | 0.02 |
| 2789391 3066770 | | | | | | | | | | |
| 3005357 | | | | | | | | | | |
| rf: 3326487 | 0.78 | 0.034 | 0.014 | 0.112 | 0.86 | 0.33 | 0.65 | 0.91 | 0.42 | 0.13 |
| 2789391 3005357 | | | | | | | | | | |
| 2651515 | | | | | | | | | | |
| rf: 3326487 | 0.76 | 0.049 | 0.074 | 0.129 | 0.86 | 0.33 | 0.62 | 0.91 | 0.47 | 0.15 |
| 3463598 3005357 | | | | | | | | | | |
| 2651515 | | | | | | | | | | |
| knn: 3326487 | 0.73 | 0.043 | 0.219 | 0.036 | 0.92 | 0.46 | 0.50 | 0.07 | 0.07 | 0.06 |
| 2461837 3066770 | | | | | | | | | | |
| 2651515 | | | | | | | | | | |
| rpart: 3326487 | 0.77 | 0.011 | 0.097 | 0.007 | 0.88 | 0.63 | 0.44 | 0.02 | 0.03 | 0.02 |
| 2512364 2364325 | | | | | | | | | | |
| 3005357 | | | | | | | | | | |
| rf: 3326487 | 0.84 | 0.008 | 0.001 | 0.018 | 0.88 | 0.35 | 0.55 | 0.44 | 0.14 | 0.04 |
| 2512364 3005357 | | | | | | | | | | |
| 2651515 | | | | | | | | | | |
| rf: 3326487 | 0.78 | 0.037 | 0.025 | 0.106 | 0.88 | 0.35 | 0.50 | 0.73 | 0.43 | 0.13 |
| 2364325 3005357 | | | | | | | | | | |
| 2651515 | | | | | | | | | | |
| rpart: 3326487 | 0.85 | 0.007 | 0.025 | 0.002 | 0.93 | 0.60 | 0.48 | 0.02 | 0.04 | 0.01 |
| 2364325 3005357 | | | | | | | | | | |
| 2651515 | | | | | | | | | | |
| rf: 3326487 | 0.77 | 0.046 | 0.051 | 0.101 | 0.89 | 0.38 | 0.54 | 0.54 | 0.37 | 0.12 |
| 3066770 3005357 | | | | | | | | | | |
| 2651515 | | | | | | | | | | |
| rpart: 3326487 | 0.85 | 0.007 | 0.025 | 0.002 | 0.93 | 0.60 | 0.48 | 0.02 | 0.04 | 0.01 |
| 3066770 3005357 | | | | | | | | | | |
| 2651515 | | | | | | | | | | |
| rf: 3326487 | 0.78 | 0.037 | 0.051 | 0.176 | 0.80 | 0.33 | 0.46 | 0.99 | 0.51 | 0.18 |
| 2956442 3005357 | | | | | | | | | | |
| 2651515 | | | | | | | | | | |
| rpart: 3326487 | 0.85 | 0.007 | 0.025 | 0.002 | 0.93 | 0.60 | 0.48 | 0.02 | 0.04 | 0.01 |
| 2956442 3005357 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 3414753 2332285 | | | | | | | | | | |
| 3005357 | | | | | | | | | | |
| knn: 2458376 | 0.75 | 0.048 | 0.457 | 0.037 | 0.55 | 0.14 | 0.50 | 0.10 | 0.04 | 0.06 |
| 3414753 2478155 | | | | | | | | | | |
| 2789391 | | | | | | | | | | |
| rpart: 2458376 | 0.77 | 0.011 | 0.097 | 0.007 | 0.88 | 0.63 | 0.44 | 0.02 | 0.03 | 0.02 |
| 3414753 3139092 | | | | | | | | | | |
| 3005357 | | | | | | | | | | |
| rf: 2458376 | 0.76 | 0.047 | 0.068 | 0.041 | 0.92 | 0.46 | 0.54 | 0.13 | 0.23 | 0.06 |
| 3414753 2512364 | | | | | | | | | | |
| 3005357 | | | | | | | | | | |
| knn: 2458376 | 0.76 | 0.048 | 0.097 | 0.031 | 0.86 | 0.45 | 0.53 | 0.39 | 0.18 | 0.05 |
| 3414753 2364325 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3414753 2364325 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3414753 3066770 | | | | | | | | | | |
| 3005357 | | | | | | | | | | |
| rf: 2458376 | 0.77 | 0.040 | 0.074 | 0.050 | 0.86 | 0.33 | 0.69 | 0.91 | 0.38 | 0.07 |
| 3414753 3005357 | | | | | | | | | | |
| 2651515 | | | | | | | | | | |
| knn: 2458376 | 0.77 | 0.041 | 0.074 | 0.017 | 0.82 | 0.50 | 0.43 | 0.36 | 0.21 | 0.03 |
| 3414753 3005357 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 2332285 2478155 | | | | | | | | | | |
| 3005357 | | | | | | | | | | |
| knn: 2458376 | 0.84 | 0.008 | 0.051 | 0.007 | 0.91 | 0.43 | 0.64 | 0.32 | 0.11 | 0.03 |
| 2332285 3139092 | | | | | | | | | | |
| 2512364 | | | | | | | | | | |
| svm: 2458376 | 0.77 | 0.041 | 0.033 | 0.024 | 0.87 | 0.50 | 0.04 | 0.11 | 0.09 | 0.04 |
| 2332285 3139092 | | | | | | | | | | |
| 2512364 | | | | | | | | | | |
| svm: 2458376 | 0.76 | 0.047 | 0.056 | 0.048 | 0.92 | 0.50 | −0.09 | 0.10 | 0.24 | 0.06 |
| 2332285 2512364 | | | | | | | | | | |
| 2956442 | | | | | | | | | | |
| rf: 2458376 | 0.76 | 0.047 | 0.043 | 0.058 | 0.88 | 0.56 | 0.47 | 0.08 | 0.31 | 0.07 |
| 2332285 2512364 | | | | | | | | | | |
| 2651515 | | | | | | | | | | |
| knn: 2458376 | 0.81 | 0.017 | 0.174 | 0.019 | 0.87 | 0.50 | 0.50 | 0.19 | 0.15 | 0.03 |
| 2332285 2364325 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 2332285 3066770 | | | | | | | | | | |
| 3005357 | | | | | | | | | | |
| rf: 2458376 | 0.85 | 0.008 | 0.007 | 0.005 | 0.93 | 0.60 | 0.45 | 0.02 | 0.10 | 0.03 |
| 2332285 3005357 | | | | | | | | | | |
| 2651515 | | | | | | | | | | |
| rpart: 2458376 | 0.79 | 0.025 | 0.097 | 0.044 | 0.91 | 0.43 | 0.56 | 0.16 | 0.12 | 0.06 |
| 2332285 3005357 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 2478155 2789391 | | | | | | | | | | |
| 3005357 | | | | | | | | | | |
| rf: 2458376 | 0.76 | 0.049 | 0.051 | 0.090 | 0.93 | 0.55 | 0.55 | 0.03 | 0.28 | 0.10 |
| 2478155 2512364 | | | | | | | | | | |
| 2651515 | | | | | | | | | | |
| knn: 2458376 | 0.79 | 0.031 | 0.051 | 0.022 | 0.87 | 0.50 | 0.51 | 0.19 | 0.11 | 0.04 |
| 2478155 2364325 | | | | | | | | | | |
| 3005357 | | | | | | | | | | |
| knn: 2458376 | 0.78 | 0.034 | 0.051 | 0.051 | 0.90 | 0.40 | 0.61 | 0.75 | 0.47 | 0.07 |
| 2478155 3066770 | | | | | | | | | | |
| 2651515 | | | | | | | | | | |
| rf: 2458376 | 0.76 | 0.047 | 0.095 | 0.047 | 0.86 | 0.33 | 0.63 | 0.91 | 0.37 | 0.07 |
| 2478155 3005357 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 3139092 2789391 | | | | | | | | | | |
| 3005357 | | | | | | | | | | |
| knn: 2458376 | 0.86 | 0.005 | 0.046 | 0.004 | 0.88 | 0.63 | 0.50 | 0.06 | 0.01 | 0.02 |
| 3139092 3463598 | | | | | | | | | | |
| 2956442 | | | | | | | | | | |
| svm: 2458376 | 0.81 | 0.017 | 0.020 | 0.026 | 0.93 | 0.55 | 0.04 | 0.04 | 0.08 | 0.04 |
| 3139092 3463598 | | | | | | | | | | |
| 2956442 | | | | | | | | | | |
| rf: 2458376 | 0.77 | 0.043 | 0.016 | 0.058 | 0.90 | 0.40 | 0.56 | 0.17 | 0.23 | 0.08 |
| 3139092 3463598 | | | | | | | | | | |
| 2651515 | | | | | | | | | | |
| knn: 2458376 | 0.78 | 0.035 | 0.137 | 0.012 | 0.91 | 0.43 | 0.61 | 0.15 | 0.07 | 0.03 |
| 3139092 2512364 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3139092 2364325 | | | | | | | | | | |
| 3005357 | | | | | | | | | | |
| knn: 2458376 | 0.80 | 0.024 | 0.097 | 0.024 | 0.80 | 0.60 | 0.35 | 0.25 | 0.22 | 0.04 |
| 3139092 2364325 | | | | | | | | | | |
| 2651515 | | | | | | | | | | |
| svm: 2458376 | 0.76 | 0.047 | 0.068 | 0.066 | 0.85 | 0.42 | −0.13 | 0.75 | 0.48 | 0.08 |
| 3139092 2364325 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 3139092 2956442 | | | | | | | | | | |
| 3005357 | | | | | | | | | | |
| rf: 2458376 | 0.76 | 0.047 | 0.014 | 0.066 | 0.85 | 0.42 | 0.44 | 0.58 | 0.33 | 0.08 |
| 2789391 3005357 | | | | | | | | | | |
| 2651515 | | | | | | | | | | |
| rpart: 2458376 | 0.77 | 0.011 | 0.097 | 0.007 | 0.88 | 0.63 | 0.44 | 0.02 | 0.03 | 0.02 |
| 3463598 2512364 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3463598 2364325 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3463598 3066770 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 3463598 2956442 | | | | | | | | | | |
| 3005357 | | | | | | | | | | |
| rf: 2458376 | 0.81 | 0.017 | 0.014 | 0.019 | 0.92 | 0.50 | 0.51 | 0.16 | 0.22 | 0.04 |
| 3463598 3005357 | | | | | | | | | | |
| 2651515 | | | | | | | | | | |
| rf: 2458376 | 0.77 | 0.043 | 0.025 | 0.033 | 0.86 | 0.45 | 0.50 | 0.18 | 0.18 | 0.05 |
| 2461837 3005357 | | | | | | | | | | |
| 2651515 | | | | | | | | | | |
| rf: 2458376 | 0.80 | 0.023 | 0.016 | 0.030 | 0.92 | 0.50 | 0.49 | 0.12 | 0.31 | 0.05 |
| 2512364 2364325 | | | | | | | | | | |
| 2651515 | | | | | | | | | | |
| knn: 2458376 | 0.79 | 0.027 | 0.126 | 0.019 | 0.89 | 0.38 | 0.64 | 0.25 | 0.09 | 0.04 |
| 2512364 3066770 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 2512364 3066770 | | | | | | | | | | |
| 3005357 | | | | | | | | | | |
| knn: 2458376 | 0.78 | 0.033 | 0.025 | 0.067 | 0.85 | 0.42 | 0.54 | 0.34 | 0.22 | 0.08 |
| 2512364 2956442 | | | | | | | | | | |
| 3005357 | | | | | | | | | | |
| rf: 2458376 | 0.84 | 0.008 | 0.001 | 0.008 | 0.93 | 0.55 | 0.43 | 0.04 | 0.09 | 0.03 |
| 2512364 3005357 | | | | | | | | | | |
| 2651515 | | | | | | | | | | |
| knn: 2458376 | 0.78 | 0.033 | 0.051 | 0.033 | 0.86 | 0.45 | 0.50 | 0.32 | 0.26 | 0.05 |
| 2512364 3005357 | | | | | | | | | | |
| 2651515 | | | | | | | | | | |
| knn: 2458376 | 0.77 | 0.035 | 0.126 | 0.052 | 0.92 | 0.50 | 0.50 | 0.08 | 0.34 | 0.07 |
| 2364325 3066770 | | | | | | | | | | |
| 2651515 | | | | | | | | | | |
| knn: 2458376 | 0.81 | 0.021 | 0.174 | 0.027 | 0.85 | 0.42 | 0.51 | 0.34 | 0.11 | 0.04 |
| 2364325 2956442 | | | | | | | | | | |
| 3005357 | | | | | | | | | | |
| rpart: 2458376 | 0.77 | 0.011 | 0.097 | 0.007 | 0.88 | 0.63 | 0.44 | 0.02 | 0.03 | 0.02 |
| 2364325 2956442 | | | | | | | | | | |
| 3005357 | | | | | | | | | | |
| rf: 2458376 | 0.83 | 0.012 | 0.008 | 0.025 | 0.86 | 0.33 | 0.62 | 0.91 | 0.30 | 0.05 |
| 2364325 3005357 | | | | | | | | | | |
| 2651515 | | | | | | | | | | |
| knn: 2458376 | 0.77 | 0.045 | 0.051 | 0.026 | 0.88 | 0.56 | 0.45 | 0.13 | 0.18 | 0.04 |
| 2364325 3005357 | | | | | | | | | | |
| 2651515 | | | | | | | | | | |
| svm: 2458376 | 0.81 | 0.017 | 0.046 | 0.013 | 0.87 | 0.50 | −0.04 | 0.22 | 0.14 | 0.03 |
| 2364325 3005357 | | | | | | | | | | |
| 2651515 | | | | | | | | | | |
| rf: 2458376 | 0.80 | 0.021 | 0.046 | 0.039 | 0.86 | 0.33 | 0.68 | 0.91 | 0.33 | 0.06 |
| 3066770 3005357 | | | | | | | | | | |
| 2651515 | | | | | | | | | | |
| rf: 2458376 | 0.79 | 0.029 | 0.008 | 0.038 | 0.92 | 0.50 | 0.45 | 0.09 | 0.25 | 0.06 |
| 2956442 3005357 | | | | | | | | | | |
| 2651515 | | | | | | | | | | |
| svm: 3414753 | 0.78 | 0.034 | 0.085 | 0.053 | 0.83 | 0.57 | −0.12 | 0.09 | 0.03 | 0.05 |
| 2332285 3139092 | | | | | | | | | | |
| 2512364 | | | | | | | | | | |
| knn: 3414753 | 0.76 | 0.046 | 0.219 | 0.033 | 0.92 | 0.46 | 0.59 | 0.18 | 0.17 | 0.05 |
| 2332285 2512364 | | | | | | | | | | |
| 3005357 | | | | | | | | | | |
| knn: 3414753 | 0.76 | 0.040 | 0.219 | 0.041 | 0.81 | 0.44 | 0.50 | 0.28 | 0.10 | 0.05 |
| 2332285 2512364 | | | | | | | | | | |
| 2651515 | | | | | | | | | | |
| rpart: 3414753 | 0.77 | 0.011 | 0.097 | 0.007 | 0.88 | 0.63 | 0.44 | 0.02 | 0.03 | 0.02 |
| 2478155 3463598 | | | | | | | | | | |
| 3005357 | | | | | | | | | | |
| rf: 3414753 | 0.77 | 0.040 | 0.097 | 0.072 | 0.88 | 0.35 | 0.57 | 0.73 | 0.41 | 0.10 |
| 2478155 3005357 | | | | | | | | | | |
| 2651515 | | | | | | | | | | |
| rpart: 3414753 | 0.77 | 0.011 | 0.097 | 0.007 | 0.88 | 0.63 | 0.44 | 0.02 | 0.03 | 0.02 |
| 3139092 2789391 | | | | | | | | | | |
| 3005357 | | | | | | | | | | |
| knn: 3414753 | 0.78 | 0.032 | 0.219 | 0.056 | 0.92 | 0.46 | 0.61 | 0.07 | 0.06 | 0.08 |
| 3139092 2461837 | | | | | | | | | | |
| 3005357 | | | | | | | | | | |
| knn: 3414753 | 0.76 | 0.046 | 0.570 | 0.019 | 0.83 | 0.38 | 0.59 | 0.43 | 0.09 | 0.04 |
| 3139092 2364325 | | | | | | | | | | |
| 3005357 | | | | | | | | | | |
| rf: 3414753 | 0.79 | 0.027 | 0.016 | 0.015 | 0.88 | 0.63 | 0.43 | 0.06 | 0.13 | 0.03 |
| 3139092 3005357 | | | | | | | | | | |
| 2651515 | | | | | | | | | | |
| rf: 3414753 | 0.76 | 0.047 | 0.085 | 0.064 | 0.83 | 0.57 | 0.36 | 0.10 | 0.31 | 0.08 |
| 2789391 2512364 | | | | | | | | | | |
| 2651515 | | | | | | | | | | |
| rf: 3414753 | 0.79 | 0.025 | 0.020 | 0.040 | 0.75 | 0.29 | 0.82 | 0.59 | 0.29 | 0.06 |
| 2512364 3005357 | | | | | | | | | | |
| 2651515 | | | | | | | | | | |
| rpart: 3414753 | 0.77 | 0.011 | 0.097 | 0.007 | 0.88 | 0.63 | 0.44 | 0.02 | 0.03 | 0.02 |
| 2364325 3066770 | | | | | | | | | | |
| 3005357 | | | | | | | | | | |
| rf: 3414753 | 0.77 | 0.042 | 0.025 | 0.108 | 0.88 | 0.35 | 0.49 | 0.73 | 0.48 | 0.14 |
| 2364325 3005357 | | | | | | | | | | |
| 2651515 | | | | | | | | | | |
| rf: 3414753 | 0.78 | 0.034 | 0.033 | 0.050 | 0.86 | 0.33 | 0.69 | 0.91 | 0.32 | 0.07 |
| 3066770 3005357 | | | | | | | | | | |
| 2651515 | | | | | | | | | | |
| knn: 3414753 | 0.75 | 0.027 | 0.174 | 0.021 | 0.88 | 0.56 | 0.50 | 0.09 | 0.10 | 0.03 |
| 3066770 3005357 | | | | | | | | | | |
| 2651515 | | | | | | | | | | |
| rpart: 3414753 | 0.85 | 0.007 | 0.025 | 0.002 | 0.93 | 0.60 | 0.48 | 0.02 | 0.04 | 0.01 |
| 3066770 3005357 | | | | | | | | | | |
| 2651515 | | | | | | | | | | |
| rpart: 2332285 | 0.82 | 0.007 | 0.068 | 0.004 | 0.93 | 0.55 | 0.55 | 0.01 | 0.01 | 0.02 |
| 2478155 2512364 | | | | | | | | | | |
| 3005357 | | | | | | | | | | |
| knn: 2332285 | 0.77 | 0.044 | 0.292 | 0.035 | 0.87 | 0.50 | 0.50 | 0.20 | 0.15 | 0.05 |
| 3139092 2789391 | | | | | | | | | | |
| 2512364 | | | | | | | | | | |
| rpart: 2332285 | 0.77 | 0.011 | 0.097 | 0.007 | 0.88 | 0.63 | 0.44 | 0.02 | 0.03 | 0.02 |
| 3139092 2789391 | | | | | | | | | | |
| 3005357 | | | | | | | | | | |
| svm: 2332285 | 0.77 | 0.041 | 0.043 | 0.058 | 0.89 | 0.38 | 0.78 | 0.22 | 0.24 | 0.07 |
| 3139092 3463598 | | | | | | | | | | |
| 2461837 | | | | | | | | | | |
| svm: 2332285 | 0.76 | 0.047 | 0.095 | 0.029 | 0.82 | 0.50 | 0.10 | 0.22 | 0.06 | 0.04 |
| 3139092 3463598 | | | | | | | | | | |
| 2512364 | | | | | | | | | | |
| knn: 2332285 | 0.75 | 0.050 | 0.174 | 0.065 | 0.90 | 0.40 | 0.70 | 0.13 | 0.20 | 0.08 |
| 3139092 3463598 | | | | | | | | | | |
| 2956442 | | | | | | | | | | |
| knn: 2332285 | 0.78 | 0.035 | 0.292 | 0.027 | 0.82 | 0.50 | 0.50 | 0.24 | 0.18 | 0.04 |
| 3139092 3463598 | | | | | | | | | | |
| 3005357 | | | | | | | | | | |
| rpart: 2332285 | 0.77 | 0.011 | 0.097 | 0.007 | 0.88 | 0.63 | 0.44 | 0.02 | 0.03 | 0.02 |
| 3139092 3463598 | | | | | | | | | | |
| 3005357 | | | | | | | | | | |
| svm: 2332285 | 0.78 | 0.034 | 0.043 | 0.021 | 0.92 | 0.50 | 0.20 | 0.01 | 0.02 | 0.05 |
| 3139092 2461837 | | | | | | | | | | |
| 2512364 | | | | | | | | | | |
| svm: 2332285 | 0.79 | 0.029 | 0.043 | 0.043 | 0.77 | 0.67 | −0.61 | 0.27 | 0.14 | 0.07 |
| 3139092 2461837 | | | | | | | | | | |
| 2651515 | | | | | | | | | | |
| knn: 2332285 | 0.77 | 0.044 | 0.097 | 0.033 | 0.87 | 0.50 | 0.52 | 0.19 | 0.21 | 0.05 |
| 3139092 2512364 | | | | | | | | | | |
| 2364325 | | | | | | | | | | |
| svm: 2332285 | 0.77 | 0.041 | 0.130 | 0.026 | 0.80 | 0.60 | −0.78 | 0.18 | 0.09 | 0.05 |
| 3139092 2512364 | | | | | | | | | | |
| 2364325 | | | | | | | | | | |
| svm: 2332285 | 0.78 | 0.034 | 0.068 | 0.043 | 0.83 | 0.57 | 0.03 | 0.09 | 0.03 | 0.05 |
| 3139092 2512364 | | | | | | | | | | |
| 3005357 | | | | | | | | | | |
| svm: 2332285 | 0.78 | 0.034 | 0.056 | 0.039 | 0.86 | 0.45 | −0.14 | 0.30 | 0.06 | 0.05 |
| 3139092 2512364 | | | | | | | | | | |
| 2651515 | | | | | | | | | | |
| knn: 2332285 | 0.77 | 0.038 | 0.126 | 0.041 | 0.78 | 0.43 | 0.44 | 0.65 | 0.29 | 0.06 |
| 3139092 2364325 | | | | | | | | | | |
| 2651515 | | | | | | | | | | |
| knn: 2332285 | 0.77 | 0.040 | 0.457 | 0.029 | 0.86 | 0.45 | 0.50 | 0.35 | 0.19 | 0.05 |
| 3139092 2956442 | | | | | | | | | | |
| 2651515 | | | | | | | | | | |
| rpart: 2332285 | 0.77 | 0.011 | 0.097 | 0.007 | 0.88 | 0.63 | 0.44 | 0.02 | 0.03 | 0.02 |
| 2789391 3463598 | | | | | | | | | | |
| 3005357 | | | | | | | | | | |
| svm: 2332285 | 0.83 | 0.012 | 0.020 | 0.013 | 0.92 | 0.46 | −0.08 | 0.15 | 0.07 | 0.04 |
| 2789391 2512364 | | | | | | | | | | |
| 2364325 | | | | | | | | | | |
| rpart: 2332285 | 0.77 | 0.011 | 0.097 | 0.007 | 0.88 | 0.63 | 0.44 | 0.02 | 0.03 | 0.02 |
| 3463598 2956442 | | | | | | | | | | |
| 3005357 | | | | | | | | | | |
| rf: 2332285 | 0.78 | 0.034 | 0.051 | 0.031 | 0.87 | 0.50 | 0.44 | 0.19 | 0.21 | 0.05 |
| 3463598 3005357 | | | | | | | | | | |
| 2651515 | | | | | | | | | | |
| rpart: 2332285 | 0.77 | 0.011 | 0.097 | 0.007 | 0.88 | 0.63 | 0.44 | 0.02 | 0.03 | 0.02 |
| 2512364 3066770 | | | | | | | | | | |
| 3005357 | | | | | | | | | | |
| rpart: 2332285 | 0.77 | 0.011 | 0.097 | 0.007 | 0.88 | 0.63 | 0.44 | 0.02 | 0.03 | 0.02 |
| 2512364 2956442 | | | | | | | | | | |
| 3005357 | | | | | | | | | | |
| rf: 2332285 | 0.80 | 0.023 | 0.016 | 0.017 | 0.93 | 0.60 | 0.41 | 0.02 | 0.17 | 0.03 |
| 2512364 3005357 | | | | | | | | | | |
| 2651515 | | | | | | | | | | |
| knn: 2332285 | 0.82 | 0.008 | 0.016 | 0.017 | 0.94 | 0.67 | 0.50 | 0.00 | 0.10 | 0.03 |
| 2512364 3005357 | | | | | | | | | | |
| 2651515 | | | | | | | | | | |
| rpart: 2332285 | 0.77 | 0.011 | 0.097 | 0.007 | 0.88 | 0.63 | 0.44 | 0.02 | 0.03 | 0.02 |
| 2364325 3066770 | | | | | | | | | | |
| 3005357 | | | | | | | | | | |
| rpart: 2332285 | 0.85 | 0.007 | 0.025 | 0.002 | 0.93 | 0.60 | 0.48 | 0.02 | 0.04 | 0.01 |
| 2364325 3005357 | | | | | | | | | | |
| 2651515 | | | | | | | | | | |
| rpart: 2332285 | 0.85 | 0.007 | 0.025 | 0.002 | 0.93 | 0.60 | 0.48 | 0.02 | 0.04 | 0.01 |
| 2956442 3005357 | | | | | | | | | | |
| 2651515 | | | | | | | | | | |
| rpart: 2478155 | 0.77 | 0.011 | 0.097 | 0.007 | 0.88 | 0.63 | 0.44 | 0.02 | 0.03 | 0.02 |
| 3139092 2789391 | | | | | | | | | | |
| 3005357 | | | | | | | | | | |
| svm: 2478155 | 0.78 | 0.034 | 0.043 | 0.168 | 1.00 | 0.29 | 5.96 | 0.00 | 0.01 | 0.11 |
| 3139092 3463598 | | | | | | | | | | |
| 2956442 | | | | | | | | | | |
| rpart: 2478155 | 0.77 | 0.011 | 0.097 | 0.007 | 0.88 | 0.63 | 0.44 | 0.02 | 0.03 | 0.02 |
| 3139092 3463598 | | | | | | | | | | |
| 3005357 | | | | | | | | | | |
| knn: 2478155 | 0.80 | 0.024 | 0.236 | 0.009 | 0.80 | 0.60 | 0.44 | 0.25 | 0.12 | 0.03 |
| 3139092 2364325 | | | | | | | | | | |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 2789391 3066770 | | | | | | | | | | |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 2789391 2956442 | | | | | | | | | | |
| 3005357 | | | | | | | | | | |
| rf: 2478155 | 0.78 | 0.034 | 0.014 | 0.094 | 0.88 | 0.35 | 0.48 | 0.73 | 0.44 | 0.12 |
| 2789391 3005357 | | | | | | | | | | |
| 2651515 | | | | | | | | | | |
| rpart: 2478155 | 0.77 | 0.011 | 0.097 | 0.007 | 0.88 | 0.63 | 0.44 | 0.02 | 0.03 | 0.02 |
| 3463598 2512364 | | | | | | | | | | |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 3463598 3066770 | | | | | | | | | | |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 3463598 2956442 | | | | | | | | | | |
| 3005357 | | | | | | | | | | |
| rf: 2478155 | 0.80 | 0.023 | 0.034 | 0.048 | 0.89 | 0.38 | 0.60 | 0.32 | 0.32 | 0.07 |
| 3463598 3005357 | | | | | | | | | | |
| 2651515 | | | | | | | | | | |
| svm: 2478155 | 0.76 | 0.047 | 0.130 | 0.029 | 0.89 | 0.38 | 0.30 | 0.26 | 0.09 | 0.05 |
| 2461837 2512364 | | | | | | | | | | |
| 3066770 | | | | | | | | | | |
| rpart: 2478155 | 0.77 | 0.036 | 0.068 | 0.072 | 0.79 | 0.50 | 0.43 | 0.30 | 0.12 | 0.08 |
| 2512364 2364325 | | | | | | | | | | |
| 3005357 | | | | | | | | | | |
| svm: 2478155 | 0.78 | 0.034 | 0.043 | 0.056 | 0.83 | 0.32 | 2.30 | 0.71 | 0.29 | 0.08 |
| 2512364 2364325 | | | | | | | | | | |
| 2651515 | | | | | | | | | | |
| rpart: 2478155 | 0.76 | 0.045 | 0.068 | 0.056 | 0.88 | 0.35 | 0.62 | 0.48 | 0.21 | 0.08 |
| 2512364 2956442 | | | | | | | | | | |
| 2651515 | | | | | | | | | | |
| rf: 2478155 | 0.83 | 0.009 | 0.001 | 0.013 | 0.93 | 0.55 | 0.46 | 0.03 | 0.14 | 0.03 |
| 2512364 3005357 | | | | | | | | | | |
| 2651515 | | | | | | | | | | |
| knn: 2478155 | 0.76 | 0.042 | 0.068 | 0.045 | 0.93 | 0.55 | 0.50 | 0.04 | 0.08 | 0.06 |
| 2512364 3005357 | | | | | | | | | | |
| 2651515 | | | | | | | | | | |
| rpart: 2478155 | 0.81 | 0.018 | 0.007 | 0.007 | 0.93 | 0.55 | 0.53 | 0.06 | 0.05 | 0.02 |
| 2512364 3005357 | | | | | | | | | | |
| 2651515 | | | | | | | | | | |
| svm: 2478155 | 0.80 | 0.021 | 0.008 | 0.027 | 0.92 | 0.50 | 0.18 | 0.10 | 0.19 | 0.04 |
| 2512364 3005357 | | | | | | | | | | |
| 2651515 | | | | | | | | | | |
| rpart: 2478155 | 0.81 | 0.010 | 0.097 | 0.006 | 0.92 | 0.50 | 0.56 | 0.03 | 0.02 | 0.02 |
| 2364325 3066770 | | | | | | | | | | |
| 3005357 | | | | | | | | | | |
| rpart: 2478155 | 0.77 | 0.011 | 0.097 | 0.007 | 0.88 | 0.63 | 0.44 | 0.02 | 0.03 | 0.02 |
| 2364325 2956442 | | | | | | | | | | |
| 3005357 | | | | | | | | | | |
| rf: 2478155 | 0.83 | 0.012 | 0.014 | 0.076 | 0.88 | 0.35 | 0.55 | 0.73 | 0.41 | 0.11 |
| 2364325 3005357 | | | | | | | | | | |
| 2651515 | | | | | | | | | | |
| svm: 2478155 | 0.77 | 0.041 | 0.008 | 0.073 | 0.91 | 0.43 | 0.16 | 0.21 | 0.56 | 0.09 |
| 2364325 3005357 | | | | | | | | | | |
| 2651515 | | | | | | | | | | |
| rpart: 2478155 | 0.81 | 0.010 | 0.097 | 0.006 | 0.92 | 0.50 | 0.56 | 0.03 | 0.02 | 0.02 |
| 3066770 2956442 | | | | | | | | | | |
| 3005357 | | | | | | | | | | |
| rf: 2478155 | 0.81 | 0.021 | 0.034 | 0.045 | 0.86 | 0.33 | 0.66 | 0.91 | 0.32 | 0.07 |
| 3066770 3005357 | | | | | | | | | | |
| 2651515 | | | | | | | | | | |
| knn: 2478155 | 0.77 | 0.038 | 0.097 | 0.075 | 0.88 | 0.35 | 0.58 | 0.73 | 0.40 | 0.09 |
| 3066770 3005357 | | | | | | | | | | |
| 2651515 | | | | | | | | | | |
| rpart: 2478155 | 0.77 | 0.040 | 0.034 | 0.011 | 0.92 | 0.46 | 0.61 | 0.09 | 0.03 | 0.03 |
| 2956442 3005357 | | | | | | | | | | |
| 2651515 | | | | | | | | | | |
| knn: 3139092 | 0.77 | 0.042 | 0.097 | 0.038 | 0.86 | 0.45 | 0.50 | 0.16 | 0.15 | 0.05 |
| 2789391 2512364 | | | | | | | | | | |
| 2364325 | | | | | | | | | | |
| knn: 3139092 | 0.78 | 0.034 | 0.137 | 0.028 | 0.82 | 0.36 | 0.54 | 0.45 | 0.10 | 0.05 |
| 2789391 2364325 | | | | | | | | | | |
| 3005357 | | | | | | | | | | |
| knn: 3139092 | 0.75 | 0.027 | 0.174 | 0.021 | 0.88 | 0.56 | 0.50 | 0.05 | 0.06 | 0.03 |
| 3463598 2461837 | | | | | | | | | | |
| 3066770 | | | | | | | | | | |
| svm: 3139092 | 0.76 | 0.047 | 0.130 | 0.043 | 0.92 | 0.46 | 0.21 | 0.09 | 0.22 | 0.06 |
| 3463598 2461837 | | | | | | | | | | |
| 3066770 | | | | | | | | | | |
| rpart: 3139092 | 0.77 | 0.011 | 0.097 | 0.007 | 0.88 | 0.63 | 0.44 | 0.02 | 0.03 | 0.02 |
| 3463598 2364325 | | | | | | | | | | |
| 3005357 | | | | | | | | | | |
| svm: 3139092 | 0.79 | 0.029 | 0.056 | 0.033 | 0.92 | 0.46 | 0.06 | 0.14 | 0.17 | 0.05 |
| 3463598 2956442 | | | | | | | | | | |
| 2651515 | | | | | | | | | | |
| svm: 3139092 | 0.76 | 0.047 | 0.174 | 0.065 | 0.82 | 0.36 | 0.16 | 0.81 | 0.29 | 0.08 |
| 2461837 2364325 | | | | | | | | | | |
| 2651515 | | | | | | | | | | |
| svm: 3139092 | 0.79 | 0.029 | 0.008 | 0.103 | 0.83 | 0.32 | 0.63 | 0.93 | 0.50 | 0.12 |
| 2461837 2956442 | | | | | | | | | | |
| 2651515 | | | | | | | | | | |
| knn: 3139092 | 0.77 | 0.041 | 0.292 | 0.028 | 0.87 | 0.50 | 0.51 | 0.19 | 0.11 | 0.04 |
| 2512364 2364325 | | | | | | | | | | |
| 3005357 | | | | | | | | | | |
| svm: 3139092 | 0.79 | 0.025 | 0.012 | 0.018 | 1.00 | 0.50 | 0.17 | 0.01 | 0.04 | 0.05 |
| 2364325 3005357 | | | | | | | | | | |
| 2651515 | | | | | | | | | | |
| rpart: 3139092 | 0.77 | 0.011 | 0.097 | 0.007 | 0.88 | 0.63 | 0.44 | 0.02 | 0.03 | 0.02 |
| 3066770 2956442 | | | | | | | | | | |
| 3005357 | | | | | | | | | | |
| rf: 2789391 | 0.77 | 0.041 | 0.020 | 0.072 | 0.91 | 0.43 | 0.46 | 0.29 | 0.18 | 0.09 |
| 3463598 2512364 | | | | | | | | | | |
| 3005357 | | | | | | | | | | |
| rpart: 2789391 | 0.77 | 0.011 | 0.097 | 0.007 | 0.88 | 0.63 | 0.44 | 0.02 | 0.03 | 0.02 |
| 3463598 3066770 | | | | | | | | | | |
| 3005357 | | | | | | | | | | |
| rf: 2789391 | 0.79 | 0.029 | 0.014 | 0.036 | 0.83 | 0.38 | 0.50 | 0.48 | 0.25 | 0.06 |
| 3463598 3005357 | | | | | | | | | | |
| 2651515 | | | | | | | | | | |
| knn: 2789391 | 0.79 | 0.030 | 0.097 | 0.026 | 0.87 | 0.50 | 0.41 | 0.09 | 0.16 | 0.04 |
| 3463598 3005357 | | | | | | | | | | |
| 2651515 | | | | | | | | | | |
| rf: 2789391 | 0.81 | 0.020 | 0.003 | 0.012 | 0.94 | 0.86 | 0.26 | 0.00 | 0.14 | 0.03 |
| 2512364 3005357 | | | | | | | | | | |
| 2651515 | | | | | | | | | | |
| svm: 2789391 | 0.77 | 0.041 | 0.033 | 0.023 | 0.85 | 0.42 | −0.10 | 0.30 | 0.18 | 0.04 |
| 2364325 3066770 | | | | | | | | | | |
| 2651515 | | | | | | | | | | |
| rpart: 2789391 | 0.77 | 0.011 | 0.097 | 0.007 | 0.88 | 0.63 | 0.44 | 0.02 | 0.03 | 0.02 |
| 2364325 2956442 | | | | | | | | | | |
| 3005357 | | | | | | | | | | |
| rpart: 2789391 | 0.85 | 0.007 | 0.025 | 0.002 | 0.93 | 0.60 | 0.48 | 0.02 | 0.04 | 0.01 |
| 2364325 3005357 | | | | | | | | | | |
| 2651515 | | | | | | | | | | |
| svm: 2789391 | 0.79 | 0.029 | 0.005 | 0.167 | 0.83 | 0.32 | 0.07 | 0.99 | 0.65 | 0.19 |
| 2364325 3005357 | | | | | | | | | | |
| 2651515 | | | | | | | | | | |
| rpart: 2789391 | 0.77 | 0.011 | 0.097 | 0.007 | 0.88 | 0.63 | 0.44 | 0.02 | 0.03 | 0.02 |
| 3066770 2956442 | | | | | | | | | | |
| 3005357 | | | | | | | | | | |
| rpart: 2789391 | 0.85 | 0.007 | 0.025 | 0.002 | 0.93 | 0.60 | 0.48 | 0.02 | 0.04 | 0.01 |
| 3066770 3005357 | | | | | | | | | | |
| 2651515 | | | | | | | | | | |
| rf: 2789391 | 0.76 | 0.047 | 0.014 | 0.051 | 0.86 | 0.33 | 0.53 | 0.91 | 0.32 | 0.07 |
| 2956442 3005357 | | | | | | | | | | |
| 2651515 | | | | | | | | | | |
| rpart: 2789391 | 0.85 | 0.007 | 0.025 | 0.002 | 0.93 | 0.60 | 0.48 | 0.02 | 0.04 | 0.01 |
| 2956442 3005357 | | | | | | | | | | |
| 2651515 | | | | | | | | | | |
| svm: 3463598 | 0.82 | 0.014 | 0.026 | 0.049 | 1.00 | 0.44 | 0.41 | 0.15 | 0.36 | 0.07 |
| 2461837 2512364 | | | | | | | | | | |
| 3005357 | | | | | | | | | | |
| rpart: 3463598 | 0.76 | 0.044 | 0.126 | 0.050 | 0.86 | 0.45 | 0.46 | 0.12 | 0.08 | 0.06 |
| 2512364 2364325 | | | | | | | | | | |
| 3005357 | | | | | | | | | | |
| rf: 3463598 | 0.79 | 0.029 | 0.008 | 0.049 | 0.90 | 0.40 | 0.58 | 0.23 | 0.25 | 0.07 |
| 2512364 2364325 | | | | | | | | | | |
| 2651515 | | | | | | | | | | |
| rpart: 3463598 | 0.77 | 0.042 | 0.068 | 0.029 | 0.87 | 0.50 | 0.41 | 0.05 | 0.03 | 0.04 |
| 2512364 3066770 | | | | | | | | | | |
| 3005357 | | | | | | | | | | |
| rpart: 3463598 | 0.77 | 0.011 | 0.097 | 0.007 | 0.88 | 0.63 | 0.44 | 0.02 | 0.03 | 0.02 |
| 2512364 2956442 | | | | | | | | | | |
| 3005357 | | | | | | | | | | |
| rf: 3463598 | 0.83 | 0.012 | 0.003 | 0.016 | 0.94 | 0.75 | 0.41 | 0.00 | 0.17 | 0.03 |
| 2512364 3005357 | | | | | | | | | | |
| 2651515 | | | | | | | | | | |
| rpart: 3463598 | 0.81 | 0.018 | 0.007 | 0.007 | 0.93 | 0.55 | 0.53 | 0.06 | 0.05 | 0.02 |
| 2512364 3005357 | | | | | | | | | | |
| 2651515 | | | | | | | | | | |
| rpart: 3463598 | 0.77 | 0.011 | 0.097 | 0.007 | 0.88 | 0.63 | 0.44 | 0.02 | 0.03 | 0.02 |
| 2364325 2956442 | | | | | | | | | | |
| 3005357 | | | | | | | | | | |
| knn: 3463598 | 0.78 | 0.035 | 0.137 | 0.028 | 0.79 | 0.36 | 0.50 | 0.83 | 0.25 | 0.04 |
| 2364325 2956442 | | | | | | | | | | |
| 2651515 | | | | | | | | | | |
| rf: 3463598 | 0.78 | 0.034 | 0.012 | 0.054 | 0.85 | 0.42 | 0.44 | 0.37 | 0.30 | 0.07 |
| 2364325 3005357 | | | | | | | | | | |
| 2651515 | | | | | | | | | | |
| rpart: 3463598 | 0.85 | 0.007 | 0.025 | 0.002 | 0.93 | 0.60 | 0.48 | 0.02 | 0.04 | 0.01 |
| 2364325 3005357 | | | | | | | | | | |
| 2651515 | | | | | | | | | | |
| rpart: 3463598 | 0.77 | 0.011 | 0.097 | 0.007 | 0.88 | 0.63 | 0.44 | 0.02 | 0.03 | 0.02 |
| 3066770 2956442 | | | | | | | | | | |
| 3005357 | | | | | | | | | | |
| rf: 3463598 | 0.78 | 0.034 | 0.097 | 0.062 | 0.85 | 0.42 | 0.49 | 0.34 | 0.35 | 0.08 |
| 3066770 3005357 | | | | | | | | | | |
| 2651515 | | | | | | | | | | |
| svm: 2461837 | 0.77 | 0.041 | 0.111 | 0.027 | 0.80 | 0.60 | −2.92 | 0.16 | 0.16 | 0.04 |
| 2512364 3066770 | | | | | | | | | | |
| 2956442 | | | | | | | | | | |
| svm: 2461837 | 0.76 | 0.047 | 0.174 | 0.052 | 0.80 | 0.60 | −3.14 | 0.11 | 0.13 | 0.07 |
| 2512364 3066770 | | | | | | | | | | |
| 3005357 | | | | | | | | | | |
| svm: 2461837 | 0.81 | 0.017 | 0.043 | 0.017 | 0.93 | 0.55 | −0.16 | 0.02 | 0.06 | 0.04 |
| 2364325 3066770 | | | | | | | | | | |
| 2651515 | | | | | | | | | | |
| knn: 2461837 | 0.79 | 0.021 | 0.236 | 0.013 | 0.87 | 0.50 | 0.50 | 0.11 | 0.05 | 0.03 |
| 2364325 3005357 | | | | | | | | | | |
| 2651515 | | | | | | | | | | |
| svm: 2461837 | 0.79 | 0.025 | 0.068 | 0.013 | 0.85 | 0.42 | 0.04 | 0.08 | 0.04 | 0.03 |
| 2364325 3005357 | | | | | | | | | | |
| 2651515 | | | | | | | | | | |
| rf: 2512364 | 0.77 | 0.041 | 0.043 | 0.033 | 0.92 | 0.46 | 0.57 | 0.13 | 0.07 | 0.05 |
| 2364325 3066770 | | | | | | | | | | |
| 3005357 | | | | | | | | | | |
| rpart: 2512364 | 0.77 | 0.042 | 0.068 | 0.029 | 0.87 | 0.50 | 0.41 | 0.05 | 0.03 | 0.04 |
| 2364325 3066770 | | | | | | | | | | |
| 3005357 | | | | | | | | | | |
| rf: 2512364 | 0.76 | 0.049 | 0.034 | 0.070 | 0.88 | 0.35 | 0.62 | 0.73 | 0.38 | 0.09 |
| 2364325 3066770 | | | | | | | | | | |
| 2651515 | | | | | | | | | | |
| rf: 2512364 | 0.83 | 0.012 | 0.001 | 0.007 | 0.94 | 0.75 | 0.36 | 0.00 | 0.10 | 0.02 |
| 2364325 3005357 | | | | | | | | | | |
| 2651515 | | | | | | | | | | |
| svm: 2512364 | 0.79 | 0.025 | 0.043 | 0.023 | 0.88 | 0.63 | −0.25 | 0.03 | 0.13 | 0.04 |
| 2364325 3005357 | | | | | | | | | | |
| 2651515 | | | | | | | | | | |
| rf: 2512364 | 0.83 | 0.012 | 0.003 | 0.015 | 0.93 | 0.60 | 0.38 | 0.01 | 0.13 | 0.03 |
| 2956442 3005357 | | | | | | | | | | |
| 2651515 | | | | | | | | | | |
| knn: 2512364 | 0.76 | 0.043 | 0.357 | 0.045 | 0.83 | 0.32 | 0.83 | 0.73 | 0.20 | 0.07 |
| 2956442 3005357 | | | | | | | | | | |
| 2651515 | | | | | | | | | | |
| rpart: 2512364 | 0.76 | 0.045 | 0.016 | 0.023 | 0.91 | 0.43 | 0.62 | 0.29 | 0.19 | 0.04 |
| 2956442 3005357 | | | | | | | | | | |
| 2651515 | | | | | | | | | | |
| svm: 2512364 | 0.79 | 0.025 | 0.033 | 0.086 | 0.92 | 0.46 | 0.15 | 0.06 | 0.36 | 0.07 |
| 2956442 3005357 | | | | | | | | | | |
| 2651515 | | | | | | | | | | |
| rf: 2364325 | 0.81 | 0.021 | 0.025 | 0.026 | 0.88 | 0.35 | 0.61 | 0.73 | 0.22 | 0.05 |
| 3066770 3005357 | | | | | | | | | | |
| 2651515 | | | | | | | | | | |
| knn: 2364325 | 0.76 | 0.043 | 0.137 | 0.034 | 0.86 | 0.33 | 0.70 | 0.66 | 0.33 | 0.06 |
| 3066770 3005357 | | | | | | | | | | |
| 2651515 | | | | | | | | | | |
| svm: 2364325 | 0.79 | 0.029 | 0.068 | 0.020 | 0.83 | 0.38 | 0.27 | 0.71 | 0.15 | 0.04 |
| 3066770 3005357 | | | | | | | | | | |
| 2651515 | | | | | | | | | | |
| rf: 2364325 | 0.79 | 0.027 | 0.025 | 0.075 | 0.88 | 0.35 | 0.54 | 0.73 | 0.36 | 0.10 |
| 2956442 3005357 | | | | | | | | | | |
| 2651515 | | | | | | | | | | |
| rf: 3066770 | 0.77 | 0.043 | 0.025 | 0.073 | 0.86 | 0.33 | 0.64 | 0.91 | 0.36 | 0.10 |
| 2956442 3005357 | | | | | | | | | | |
| 2651515 | | | | | | | | | | |

Performance of pairwise, threewise and fourwise combinations of the 15 markers across different metrics for the Recurrence endpoint for LNI positive patients. All classifiers have a Wilcox P-value <=0.05

TABLE 15

| | | WILCOX | KS | T-TEST | | | | KM P- | UVA HR | UVA OR |
|---|---|---|---|---|---|---|---|---|---|---|
| CLASSIFIER | AUC | P-VALUE | P-VALUE | P-VALUE | PPV | NPV | CUTOFF | VALUE | P-VALUE | P-VALUE |
| rf:3326487 | 0.70 | 0.020 | 0.018 | 0.013 | 0.83 | 0.53 | 0.55 | 0.04 | 0.02 | 0.02 |
| 2458376 | | | | | | | | | | |
| nb:3326487 | 0.72 | 0.012 | 0.078 | 0.019 | 0.86 | 0.56 | 0.49 | 0.02 | 0.02 | 0.03 |
| 2458376 | | | | | | | | | | |
| knn:3326487 | 0.67 | 0.047 | 0.104 | 0.018 | 0.85 | 0.55 | 0.51 | 0.04 | 0.03 | 0.03 |
| 2458376 | | | | | | | | | | |
| rpart:3326487 | 0.75 | 0.002 | 0.014 | 0.001 | 0.77 | 0.70 | 0.41 | 0.00 | 0.00 | 0.00 |
| 2458376 | | | | | | | | | | |
| svm:3326487 | 0.71 | 0.014 | 0.078 | 0.016 | 0.80 | 0.55 | 0.18 | 0.06 | 0.04 | 0.02 |
| 2458376 | | | | | | | | | | |
| nb:3326487 | 0.73 | 0.008 | 0.018 | 0.019 | 0.80 | 0.50 | 0.54 | 0.08 | 0.01 | 0.03 |
| 3414753 | | | | | | | | | | |
| svm:3326487 | 0.67 | 0.047 | 0.148 | 0.056 | 0.67 | 0.48 | −0.18 | 0.45 | 0.04 | 0.06 |
| 3414753 | | | | | | | | | | |
| nb:3326487 | 0.76 | 0.002 | 0.004 | 0.010 | 0.80 | 0.55 | 0.48 | 0.03 | 0.01 | 0.02 |
| 2332285 | | | | | | | | | | |
| knn:3326487 | 0.68 | 0.039 | 0.081 | 0.012 | 0.91 | 0.54 | 0.53 | 0.01 | 0.01 | 0.02 |
| 2332285 | | | | | | | | | | |
| svm:3326487 | 0.72 | 0.011 | 0.063 | 0.019 | 0.69 | 0.50 | −0.21 | 0.25 | 0.01 | 0.03 |
| 2332285 | | | | | | | | | | |
| nb:3326487 | 0.74 | 0.005 | 0.036 | 0.008 | 0.80 | 0.50 | 0.54 | 0.07 | 0.00 | 0.02 |
| 2478155 | | | | | | | | | | |
| nb:3326487 | 0.70 | 0.021 | 0.014 | 0.028 | 0.71 | 0.50 | 0.49 | 0.15 | 0.01 | 0.04 |
| 3139092 | | | | | | | | | | |
| knn:3326487 | 0.70 | 0.022 | 0.014 | 0.028 | 0.69 | 0.50 | 0.52 | 0.20 | 0.03 | 0.03 |
| 3139092 | | | | | | | | | | |
| rpart:3326487 | 0.71 | 0.008 | 0.075 | 0.008 | 0.73 | 0.65 | 0.41 | 0.01 | 0.00 | 0.01 |
| 3139092 | | | | | | | | | | |
| nb:3326487 | 0.67 | 0.049 | 0.208 | 0.030 | 0.80 | 0.50 | 0.52 | 0.09 | 0.01 | 0.04 |
| 2789391 | | | | | | | | | | |
| rf:3326487 | 0.68 | 0.038 | 0.019 | 0.015 | 0.81 | 0.64 | 0.39 | 0.01 | 0.02 | 0.02 |
| 3463598 | | | | | | | | | | |
| nb:3326487 | 0.68 | 0.037 | 0.036 | 0.026 | 0.80 | 0.50 | 0.56 | 0.07 | 0.01 | 0.04 |
| 3463598 | | | | | | | | | | |
| nb:3326487 | 0.68 | 0.044 | 0.089 | 0.033 | 0.69 | 0.48 | 0.49 | 0.30 | 0.01 | 0.04 |
| 2461837 | | | | | | | | | | |
| rpart:3326487 | 0.68 | 0.019 | 0.118 | 0.017 | 0.74 | 0.61 | 0.47 | 0.01 | 0.02 | 0.02 |
| 2461837 | | | | | | | | | | |
| nb:3326487 | 0.72 | 0.012 | 0.044 | 0.013 | 0.69 | 0.48 | 0.50 | 0.34 | 0.01 | 0.02 |
| 2512364 | | | | | | | | | | |
| knn:3326487 | 0.69 | 0.032 | 0.098 | 0.031 | 0.80 | 0.50 | 0.58 | 0.07 | 0.04 | 0.04 |
| 2512364 | | | | | | | | | | |
| rf:3326487 | 0.73 | 0.008 | 0.038 | 0.006 | 0.83 | 0.53 | 0.56 | 0.02 | 0.01 | 0.01 |
| 2364325 | | | | | | | | | | |
| nb:3326487 | 0.74 | 0.006 | 0.020 | 0.007 | 0.90 | 0.53 | 0.54 | 0.01 | 0.00 | 0.02 |
| 2364325 | | | | | | | | | | |
| knn:3326487 | 0.69 | 0.030 | 0.092 | 0.032 | 0.89 | 0.51 | 0.61 | 0.01 | 0.04 | 0.04 |
| 2364325 | | | | | | | | | | |
| svm:3326487 | 0.70 | 0.023 | 0.101 | 0.032 | 0.75 | 0.50 | 0.03 | 0.16 | 0.03 | 0.04 |
| 2364325 | | | | | | | | | | |
| nb:3326487 | 0.69 | 0.030 | 0.101 | 0.039 | 0.80 | 0.50 | 0.55 | 0.07 | 0.02 | 0.05 |
| 3066770 | | | | | | | | | | |
| nb:3326487 | 0.75 | 0.003 | 0.003 | 0.015 | 0.67 | 0.47 | 0.50 | 0.40 | 0.01 | 0.03 |
| 3005357 | | | | | | | | | | |
| svm:3326487 | 0.70 | 0.018 | 0.018 | 0.036 | 0.67 | 0.48 | 0.07 | 0.30 | 0.04 | 0.04 |
| 3005357 | | | | | | | | | | |
| rpart:3326487 | 0.66 | 0.018 | 0.189 | 0.015 | 0.85 | 0.55 | 0.56 | 0.01 | 0.02 | 0.03 |
| 2651515 | | | | | | | | | | |
| rf:2458376 | 0.75 | 0.004 | 0.010 | 0.002 | 0.79 | 0.59 | 0.52 | 0.02 | 0.01 | 0.00 |
| 3414753 | | | | | | | | | | |
| nb:2458376 | 0.78 | 0.001 | 0.003 | 0.002 | 0.85 | 0.55 | 0.56 | 0.03 | 0.01 | 0.01 |
| 3414753 | | | | | | | | | | |
| rpart:2458376 | 0.65 | 0.040 | 0.261 | 0.037 | 0.76 | 0.55 | 0.52 | 0.04 | 0.04 | 0.04 |
| 3414753 | | | | | | | | | | |
| rf:2458376 | 0.76 | 0.002 | 0.001 | 0.001 | 0.80 | 0.71 | 0.42 | 0.00 | 0.01 | 0.00 |
| 2332285 | | | | | | | | | | |
| nb:2458376 | 0.80 | 0.000 | 0.001 | 0.001 | 0.83 | 0.61 | 0.48 | 0.01 | 0.00 | 0.00 |
| 2332285 | | | | | | | | | | |
| knn:2458376 | 0.70 | 0.017 | 0.098 | 0.020 | 0.76 | 0.60 | 0.50 | 0.02 | 0.02 | 0.03 |
| 2332285 | | | | | | | | | | |
| rpart:2458376 | 0.73 | 0.002 | 0.014 | 0.001 | 0.77 | 0.70 | 0.48 | 0.00 | 0.01 | 0.00 |
| 2332285 | | | | | | | | | | |
| svm:2458376 | 0.81 | 0.000 | 0.001 | 0.000 | 0.85 | 0.65 | −0.15 | 0.00 | 0.00 | 0.00 |
| 2332285 | | | | | | | | | | |
| rf:2458376 | 0.69 | 0.027 | 0.032 | 0.021 | 0.71 | 0.59 | 0.60 | 0.06 | 0.07 | 0.03 |
| 2478155 | | | | | | | | | | |
| nb:2458376 | 0.83 | 0.000 | 0.000 | 0.001 | 0.85 | 0.55 | 0.53 | 0.03 | 0.00 | 0.01 |
| 2478155 | | | | | | | | | | |
| rpart:2458376 | 0.73 | 0.002 | 0.014 | 0.001 | 0.77 | 0.70 | 0.48 | 0.00 | 0.01 | 0.00 |
| 2478155 | | | | | | | | | | |
| svm:2458376 | 0.73 | 0.008 | 0.047 | 0.005 | 0.71 | 0.56 | 0.09 | 0.12 | 0.02 | 0.01 |
| 2478155 | | | | | | | | | | |
| nb:2458376 | 0.78 | 0.001 | 0.004 | 0.003 | 0.81 | 0.57 | 0.49 | 0.03 | 0.01 | 0.01 |
| 3139092 | | | | | | | | | | |
| rpart:2458376 | 0.73 | 0.002 | 0.014 | 0.001 | 0.77 | 0.70 | 0.48 | 0.00 | 0.01 | 0.00 |
| 3139092 | | | | | | | | | | |
| svm:2458376 | 0.68 | 0.037 | 0.004 | 0.003 | 0.76 | 0.76 | 0.25 | 0.00 | 0.01 | 0.01 |
| 3139092 | | | | | | | | | | |
| rf:2458376 | 0.75 | 0.004 | 0.002 | 0.001 | 0.87 | 0.58 | 0.57 | 0.00 | 0.00 | 0.00 |
| 2789391 | | | | | | | | | | |
| nb:2458376 | 0.72 | 0.010 | 0.001 | 0.006 | 0.87 | 0.58 | 0.49 | 0.00 | 0.01 | 0.01 |
| 2789391 | | | | | | | | | | |
| rpart:2458376 | 0.73 | 0.002 | 0.014 | 0.001 | 0.77 | 0.70 | 0.48 | 0.00 | 0.01 | 0.00 |
| 2789391 | | | | | | | | | | |
| rf:2458376 | 0.75 | 0.005 | 0.001 | 0.001 | 0.85 | 0.65 | 0.54 | 0.00 | 0.00 | 0.00 |
| 3463598 | | | | | | | | | | |
| nb:2458376 | 0.70 | 0.021 | 0.001 | 0.005 | 0.93 | 0.61 | 0.51 | 0.00 | 0.01 | 0.01 |
| 3463598 | | | | | | | | | | |
| knn:2458376 | 0.73 | 0.007 | 0.001 | 0.000 | 0.94 | 0.66 | 0.54 | 0.00 | 0.00 | 0.00 |
| 3463598 | | | | | | | | | | |
| rpart:2458376 | 0.71 | 0.006 | 0.040 | 0.004 | 0.80 | 0.62 | 0.53 | 0.01 | 0.01 | 0.01 |
| 3463598 | | | | | | | | | | |
| svm:2458376 | 0.67 | 0.049 | 0.005 | 0.009 | 0.88 | 0.62 | 0.12 | 0.00 | 0.03 | 0.01 |
| 3463598 | | | | | | | | | | |
| nb:2458376 | 0.71 | 0.014 | 0.007 | 0.007 | 0.88 | 0.62 | 0.48 | 0.00 | 0.01 | 0.01 |
| 2461837 | | | | | | | | | | |
| knn:2458376 | 0.74 | 0.006 | 0.081 | 0.006 | 0.69 | 0.60 | 0.45 | 0.05 | 0.01 | 0.01 |
| 2461837 | | | | | | | | | | |
| rpart:2458376 | 0.68 | 0.019 | 0.118 | 0.017 | 0.74 | 0.61 | 0.47 | 0.01 | 0.02 | 0.02 |
| 2461837 | | | | | | | | | | |
| svm:2458376 | 0.69 | 0.028 | 0.123 | 0.037 | 0.67 | 0.55 | 0.11 | 0.13 | 0.07 | 0.04 |
| 2461837 | | | | | | | | | | |
| nb:2458376 | 0.76 | 0.002 | 0.007 | 0.003 | 0.87 | 0.58 | 0.52 | 0.01 | 0.01 | 0.01 |
| 2512364 | | | | | | | | | | |
| knn:2458376 | 0.73 | 0.008 | 0.024 | 0.004 | 0.87 | 0.58 | 0.58 | 0.01 | 0.01 | 0.01 |
| 2512364 | | | | | | | | | | |
| svm:2458376 | 0.71 | 0.015 | 0.005 | 0.024 | 0.88 | 0.62 | 0.12 | 0.00 | 0.03 | 0.03 |
| 2512364 | | | | | | | | | | |
| rf:2458376 | 0.80 | 0.001 | 0.000 | 0.000 | 0.88 | 0.62 | 0.65 | 0.00 | 0.00 | 0.00 |
| 2364325 | | | | | | | | | | |
| nb:2458376 | 0.79 | 0.001 | 0.000 | 0.001 | 0.86 | 0.56 | 0.53 | 0.01 | 0.01 | 0.01 |
| 2364325 | | | | | | | | | | |
| knn:2458376 | 0.82 | 0.000 | 0.006 | 0.000 | 0.88 | 0.60 | 0.53 | 0.00 | 0.00 | 0.00 |
| 2364325 | | | | | | | | | | |
| rpart:2458376 | 0.73 | 0.002 | 0.014 | 0.001 | 0.77 | 0.70 | 0.48 | 0.00 | 0.01 | 0.00 |
| 2364325 | | | | | | | | | | |
| svm:2458376 | 0.79 | 0.001 | 0.000 | 0.001 | 0.88 | 0.60 | 0.03 | 0.00 | 0.00 | 0.00 |
| 2364325 | | | | | | | | | | |
| rf:2458376 | 0.71 | 0.016 | 0.038 | 0.023 | 0.71 | 0.59 | 0.46 | 0.07 | 0.06 | 0.03 |
| 3066770 | | | | | | | | | | |
| nb:2458376 | 0.72 | 0.010 | 0.007 | 0.008 | 0.86 | 0.56 | 0.52 | 0.02 | 0.02 | 0.02 |
| 3066770 | | | | | | | | | | |
| knn:2458376 | 0.72 | 0.013 | 0.066 | 0.009 | 0.76 | 0.60 | 0.50 | 0.02 | 0.04 | 0.02 |
| 3066770 | | | | | | | | | | |
| rpart:2458376 | 0.73 | 0.002 | 0.014 | 0.001 | 0.77 | 0.70 | 0.48 | 0.00 | 0.01 | 0.00 |
| 3066770 | | | | | | | | | | |
| svm:2458376 | 0.74 | 0.005 | 0.013 | 0.007 | 0.79 | 0.59 | 0.20 | 0.02 | 0.03 | 0.01 |
| 3066770 | | | | | | | | | | |
| rf:2458376 | 0.73 | 0.009 | 0.011 | 0.004 | 0.81 | 0.64 | 0.48 | 0.01 | 0.02 | 0.01 |
| 2956442 | | | | | | | | | | |
| nb:2458376 | 0.68 | 0.042 | 0.007 | 0.014 | 0.83 | 0.61 | 0.46 | 0.00 | 0.03 | 0.02 |
| 2956442 | | | | | | | | | | |
| knn:2458376 | 0.75 | 0.004 | 0.019 | 0.002 | 0.83 | 0.61 | 0.55 | 0.00 | 0.01 | 0.01 |
| 2956442 | | | | | | | | | | |
| rpart:2458376 | 0.79 | 0.001 | 0.002 | 0.003 | 0.79 | 0.59 | 0.57 | 0.01 | 0.01 | 0.01 |
| 2956442 | | | | | | | | | | |
| svm:2458376 | 0.68 | 0.037 | 0.059 | 0.039 | 0.75 | 0.53 | −0.04 | 0.19 | 0.11 | 0.04 |
| 2956442 | | | | | | | | | | |
| rf:2458376 | 0.78 | 0.002 | 0.006 | 0.001 | 0.78 | 0.65 | 0.50 | 0.00 | 0.00 | 0.00 |
| 3005357 | | | | | | | | | | |
| nb:2458376 | 0.79 | 0.001 | 0.003 | 0.002 | 0.87 | 0.58 | 0.50 | 0.01 | 0.01 | 0.01 |
| 3005357 | | | | | | | | | | |
| knn:2458376 | 0.78 | 0.001 | 0.006 | 0.001 | 0.75 | 0.64 | 0.48 | 0.01 | 0.00 | 0.00 |
| 3005357 | | | | | | | | | | |
| rpart:2458376 | 0.73 | 0.002 | 0.014 | 0.001 | 0.77 | 0.70 | 0.48 | 0.00 | 0.01 | 0.00 |
| 3005357 | | | | | | | | | | |
| svm:2458376 | 0.77 | 0.002 | 0.001 | 0.001 | 0.82 | 0.59 | 0.06 | 0.01 | 0.01 | 0.00 |
| 3005357 | | | | | | | | | | |
| rf:2458376 | 0.72 | 0.013 | 0.030 | 0.007 | 0.86 | 0.56 | 0.55 | 0.01 | 0.02 | 0.01 |
| 2651515 | | | | | | | | | | |
| nb:2458376 | 0.73 | 0.006 | 0.017 | 0.010 | 0.89 | 0.51 | 0.58 | 0.09 | 0.03 | 0.02 |
| 2651515 | | | | | | | | | | |
| knn:2458376 | 0.77 | 0.002 | 0.001 | 0.001 | 0.86 | 0.71 | 0.46 | 0.00 | 0.00 | 0.00 |
| 2651515 | | | | | | | | | | |
| rpart:2458376 | 0.74 | 0.003 | 0.030 | 0.002 | 0.76 | 0.67 | 0.39 | 0.01 | 0.00 | 0.00 |
| 2651515 | | | | | | | | | | |
| svm:2458376 | 0.73 | 0.006 | 0.016 | 0.006 | 0.77 | 0.63 | −0.22 | 0.01 | 0.02 | 0.01 |
| 2651515 | | | | | | | | | | |
| rf:3414753 | 0.69 | 0.028 | 0.040 | 0.014 | 0.81 | 0.57 | 0.57 | 0.01 | 0.02 | 0.02 |
| 2332285 | | | | | | | | | | |
| nb:3414753 | 0.79 | 0.001 | 0.001 | 0.003 | 0.79 | 0.53 | 0.55 | 0.06 | 0.00 | 0.01 |
| 2332285 | | | | | | | | | | |
| rpart:3414753 | 0.65 | 0.040 | 0.261 | 0.037 | 0.76 | 0.55 | 0.52 | 0.04 | 0.04 | 0.04 |
| 2332285 | | | | | | | | | | |
| svm:3414753 | 0.75 | 0.003 | 0.002 | 0.001 | 0.85 | 0.65 | −0.01 | 0.00 | 0.00 | 0.00 |
| 2332285 | | | | | | | | | | |
| rf:3414753 | 0.75 | 0.004 | 0.006 | 0.003 | 0.78 | 0.57 | 0.67 | 0.02 | 0.01 | 0.01 |
| 2478155 | | | | | | | | | | |
| nb:3414753 | 0.80 | 0.000 | 0.001 | 0.001 | 0.81 | 0.64 | 0.49 | 0.00 | 0.00 | 0.00 |
| 2478155 | | | | | | | | | | |
| knn:3414753 | 0.71 | 0.015 | 0.118 | 0.017 | 0.63 | 0.57 | 0.40 | 0.30 | 0.03 | 0.02 |
| 2478155 | | | | | | | | | | |
| rpart:3414753 | 0.65 | 0.040 | 0.261 | 0.037 | 0.76 | 0.55 | 0.52 | 0.04 | 0.04 | 0.04 |
| 2478155 | | | | | | | | | | |
| svm:3414753 | 0.77 | 0.002 | 0.001 | 0.001 | 0.75 | 0.64 | −0.23 | 0.02 | 0.00 | 0.00 |
| 2478155 | | | | | | | | | | |
| rf:3414753 | 0.70 | 0.021 | 0.006 | 0.009 | 0.76 | 0.67 | 0.52 | 0.01 | 0.02 | 0.01 |
| 3139092 | | | | | | | | | | |
| nb:3414753 | 0.78 | 0.001 | 0.000 | 0.002 | 0.74 | 0.56 | 0.49 | 0.03 | 0.00 | 0.01 |
| 3139092 | | | | | | | | | | |
| knn:3414753 | 0.71 | 0.016 | 0.058 | 0.025 | 0.73 | 0.65 | 0.43 | 0.02 | 0.04 | 0.03 |
| 3139092 | | | | | | | | | | |
| rpart:3414753 | 0.65 | 0.040 | 0.261 | 0.037 | 0.76 | 0.55 | 0.52 | 0.04 | 0.04 | 0.04 |
| 3139092 | | | | | | | | | | |
| svm:3414753 | 0.76 | 0.002 | 0.001 | 0.001 | 0.76 | 0.67 | 0.14 | 0.01 | 0.01 | 0.00 |
| 3139092 | | | | | | | | | | |
| rf:3414753 | 0.71 | 0.017 | 0.019 | 0.009 | 0.77 | 0.63 | 0.47 | 0.01 | 0.01 | 0.01 |
| 2789391 | | | | | | | | | | |
| nb:3414753 | 0.72 | 0.012 | 0.010 | 0.006 | 0.90 | 0.53 | 0.56 | 0.00 | 0.00 | 0.01 |
| 2789391 | | | | | | | | | | |
| knn:3414753 | 0.75 | 0.004 | 0.009 | 0.001 | 0.88 | 0.62 | 0.50 | 0.00 | 0.00 | 0.00 |
| 2789391 | | | | | | | | | | |
| rpart:3414753 | 0.65 | 0.040 | 0.261 | 0.037 | 0.76 | 0.55 | 0.52 | 0.04 | 0.04 | 0.04 |
| 2789391 | | | | | | | | | | |
| svm:3414753 | 0.72 | 0.011 | 0.010 | 0.010 | 0.70 | 0.63 | −0.42 | 0.03 | 0.01 | 0.02 |
| 2789391 | | | | | | | | | | |
| rf:3414753 | 0.71 | 0.016 | 0.019 | 0.004 | 0.78 | 0.65 | 0.52 | 0.01 | 0.01 | 0.01 |
| 3463598 | | | | | | | | | | |
| nb:3414753 | 0.73 | 0.007 | 0.005 | 0.003 | 0.81 | 0.64 | 0.49 | 0.00 | 0.00 | 0.01 |
| 3463598 | | | | | | | | | | |
| knn:3414753 | 0.70 | 0.020 | 0.006 | 0.004 | 0.81 | 0.64 | 0.50 | 0.00 | 0.02 | 0.01 |
| 3463598 | | | | | | | | | | |
| rpart:3414753 | 0.71 | 0.006 | 0.040 | 0.004 | 0.80 | 0.62 | 0.53 | 0.01 | 0.01 | 0.01 |
| 3463598 | | | | | | | | | | |
| svm:3414753 | 0.73 | 0.009 | 0.005 | 0.004 | 0.82 | 0.67 | 0.04 | 0.00 | 0.01 | 0.01 |
| 3463598 | | | | | | | | | | |
| nb:3414753 | 0.74 | 0.005 | 0.007 | 0.004 | 0.77 | 0.63 | 0.45 | 0.00 | 0.00 | 0.01 |
| 2461837 | | | | | | | | | | |
| rpart:3414753 | 0.69 | 0.021 | 0.118 | 0.014 | 0.78 | 0.57 | 0.57 | 0.01 | 0.01 | 0.02 |
| 2461837 | | | | | | | | | | |
| nb:3414753 | 0.75 | 0.003 | 0.000 | 0.002 | 0.83 | 0.70 | 0.48 | 0.00 | 0.00 | 0.01 |
| 2512364 | | | | | | | | | | |
| knn:3414753 | 0.77 | 0.002 | 0.001 | 0.001 | 0.89 | 0.64 | 0.55 | 0.00 | 0.00 | 0.00 |
| 2512364 | | | | | | | | | | |
| rpart:3414753 | 0.65 | 0.040 | 0.261 | 0.037 | 0.76 | 0.55 | 0.52 | 0.04 | 0.04 | 0.04 |
| 2512364 | | | | | | | | | | |
| svm:3414753 | 0.76 | 0.003 | 0.000 | 0.001 | 0.86 | 0.68 | 0.00 | 0.00 | 0.00 | 0.00 |
| 2512364 | | | | | | | | | | |
| rf:3414753 | 0.73 | 0.010 | 0.011 | 0.002 | 0.78 | 0.65 | 0.50 | 0.01 | 0.01 | 0.00 |
| 2364325 | | | | | | | | | | |
| nb:3414753 | 0.78 | 0.001 | 0.001 | 0.001 | 0.83 | 0.70 | 0.45 | 0.00 | 0.00 | 0.00 |
| 2364325 | | | | | | | | | | |
| knn:3414753 | 0.75 | 0.003 | 0.012 | 0.001 | 0.83 | 0.61 | 0.48 | 0.00 | 0.00 | 0.00 |
| 2364325 | | | | | | | | | | |
| rpart:3414753 | 0.65 | 0.040 | 0.261 | 0.037 | 0.76 | 0.55 | 0.52 | 0.04 | 0.04 | 0.04 |
| 2364325 | | | | | | | | | | |
| svm:3414753 | 0.67 | 0.049 | 0.026 | 0.041 | 0.68 | 0.52 | −0.05 | 0.20 | 0.06 | 0.05 |
| 2364325 | | | | | | | | | | |
| rf:3414753 | 0.67 | 0.046 | 0.118 | 0.026 | 0.74 | 0.56 | 0.53 | 0.06 | 0.03 | 0.03 |
| 3066770 | | | | | | | | | | |
| nb:3414753 | 0.71 | 0.013 | 0.020 | 0.006 | 0.82 | 0.59 | 0.50 | 0.00 | 0.00 | 0.01 |
| 3066770 | | | | | | | | | | |
| knn:3414753 | 0.73 | 0.007 | 0.066 | 0.010 | 0.81 | 0.57 | 0.57 | 0.01 | 0.01 | 0.02 |
| 3066770 | | | | | | | | | | |
| rpart:3414753 | 0.65 | 0.040 | 0.261 | 0.037 | 0.76 | 0.55 | 0.52 | 0.04 | 0.04 | 0.04 |
| 3066770 | | | | | | | | | | |
| rf:3414753 | 0.70 | 0.019 | 0.019 | 0.013 | 0.80 | 0.62 | 0.42 | 0.00 | 0.02 | 0.02 |
| 2956442 | | | | | | | | | | |
| nb:3414753 | 0.72 | 0.012 | 0.016 | 0.012 | 0.77 | 0.63 | 0.45 | 0.01 | 0.02 | 0.02 |
| 2956442 | | | | | | | | | | |
| knn:3414753 | 0.73 | 0.009 | 0.011 | 0.012 | 0.78 | 0.57 | 0.50 | 0.01 | 0.01 | 0.02 |
| 2956442 | | | | | | | | | | |
| rpart:3414753 | 0.65 | 0.040 | 0.261 | 0.037 | 0.76 | 0.55 | 0.52 | 0.04 | 0.04 | 0.04 |
| 2956442 | | | | | | | | | | |
| svm:3414753 | 0.73 | 0.008 | 0.016 | 0.010 | 0.79 | 0.59 | −0.23 | 0.01 | 0.01 | 0.02 |
| 2956442 | | | | | | | | | | |
| rf:3414753 | 0.74 | 0.006 | 0.022 | 0.004 | 0.79 | 0.59 | 0.50 | 0.01 | 0.01 | 0.01 |
| 3005357 | | | | | | | | | | |
| nb:3414753 | 0.77 | 0.002 | 0.001 | 0.002 | 0.78 | 0.65 | 0.46 | 0.00 | 0.00 | 0.00 |
| 3005357 | | | | | | | | | | |
| knn:3414753 | 0.79 | 0.001 | 0.005 | 0.001 | 0.77 | 0.63 | 0.50 | 0.01 | 0.00 | 0.00 |
| 3005357 | | | | | | | | | | |
| rpart:3414753 | 0.65 | 0.040 | 0.261 | 0.037 | 0.76 | 0.55 | 0.52 | 0.04 | 0.04 | 0.04 |
| 3005357 | | | | | | | | | | |
| rf:3414753 | 0.68 | 0.038 | 0.054 | 0.010 | 0.86 | 0.56 | 0.58 | 0.01 | 0.02 | 0.02 |
| 2651515 | | | | | | | | | | |
| nb:3414753 | 0.74 | 0.005 | 0.051 | 0.006 | 0.85 | 0.55 | 0.50 | 0.02 | 0.01 | 0.02 |
| 2651515 | | | | | | | | | | |
| rpart:3414753 | 0.66 | 0.018 | 0.189 | 0.015 | 0.85 | 0.55 | 0.56 | 0.01 | 0.02 | 0.03 |
| 2651515 | | | | | | | | | | |
| rf:2332285 | 0.75 | 0.004 | 0.013 | 0.003 | 0.76 | 0.67 | 0.57 | 0.01 | 0.02 | 0.01 |
| 2478155 | | | | | | | | | | |
| nb:2332285 | 0.82 | 0.000 | 0.000 | 0.001 | 0.85 | 0.65 | 0.46 | 0.00 | 0.00 | 0.00 |
| 2478155 | | | | | | | | | | |
| knn:2332285 | 0.70 | 0.019 | 0.003 | 0.002 | 0.85 | 0.65 | 0.51 | 0.00 | 0.01 | 0.01 |
| 2478155 | | | | | | | | | | |
| rpart:2332285 | 0.71 | 0.013 | 0.086 | 0.011 | 0.64 | 0.56 | 0.55 | 0.25 | 0.03 | 0.02 |
| 2478155 | | | | | | | | | | |
| svm:2332285 | 0.69 | 0.030 | 0.033 | 0.011 | 0.71 | 0.56 | −0.14 | 0.10 | 0.01 | 0.02 |
| 2478155 | | | | | | | | | | |
| rf:2332285 | 0.75 | 0.005 | 0.006 | 0.002 | 0.78 | 0.74 | 0.43 | 0.00 | 0.00 | 0.00 |
| 3139092 | | | | | | | | | | |
| nb:2332285 | 0.80 | 0.000 | 0.000 | 0.002 | 0.80 | 0.62 | 0.49 | 0.01 | 0.00 | 0.01 |
| 3139092 | | | | | | | | | | |
| knn:2332285 | 0.72 | 0.013 | 0.001 | 0.002 | 0.81 | 0.64 | 0.50 | 0.00 | 0.01 | 0.00 |
| 3139092 | | | | | | | | | | |
| rpart:2332285 | 0.72 | 0.004 | 0.030 | 0.003 | 0.76 | 0.67 | 0.49 | 0.00 | 0.01 | 0.01 |
| 3139092 | | | | | | | | | | |
| svm:2332285 | 0.78 | 0.001 | 0.001 | 0.000 | 0.78 | 0.74 | −0.11 | 0.00 | 0.00 | 0.00 |
| 3139092 | | | | | | | | | | |
| rf:2332285 | 0.72 | 0.011 | 0.012 | 0.005 | 0.87 | 0.58 | 0.54 | 0.00 | 0.01 | 0.01 |
| 2789391 | | | | | | | | | | |
| nb:2332285 | 0.75 | 0.004 | 0.001 | 0.004 | 0.84 | 0.63 | 0.45 | 0.00 | 0.00 | 0.01 |
| 2789391 | | | | | | | | | | |
| knn:2332285 | 0.74 | 0.005 | 0.018 | 0.001 | 0.82 | 0.59 | 0.51 | 0.01 | 0.00 | 0.00 |
| 2789391 | | | | | | | | | | |
| svm:2332285 | 0.68 | 0.040 | 0.047 | 0.010 | 0.86 | 0.56 | −0.01 | 0.01 | 0.01 | 0.02 |
| 2789391 | | | | | | | | | | |
| rf:2332285 | 0.76 | 0.003 | 0.006 | 0.001 | 0.78 | 0.65 | 0.42 | 0.01 | 0.00 | 0.00 |
| 3463598 | | | | | | | | | | |
| nb:2332285 | 0.75 | 0.003 | 0.001 | 0.002 | 0.86 | 0.68 | 0.47 | 0.00 | 0.00 | 0.01 |
| 3463598 | | | | | | | | | | |
| knn:2332285 | 0.71 | 0.013 | 0.104 | 0.015 | 0.86 | 0.56 | 0.64 | 0.01 | 0.03 | 0.02 |
| 3463598 | | | | | | | | | | |
| rpart:2332285 | 0.71 | 0.006 | 0.040 | 0.004 | 0.80 | 0.62 | 0.53 | 0.01 | 0.01 | 0.01 |
| 3463598 | | | | | | | | | | |
| svm:2332285 | 0.76 | 0.003 | 0.000 | 0.000 | 0.85 | 0.65 | 0.04 | 0.00 | 0.00 | 0.00 |
| 3463598 | | | | | | | | | | |
| rf:2332285 | 0.72 | 0.011 | 0.019 | 0.019 | 0.67 | 0.58 | 0.39 | 0.10 | 0.02 | 0.02 |
| 2461837 | | | | | | | | | | |
| nb:2332285 | 0.75 | 0.003 | 0.001 | 0.002 | 0.85 | 0.65 | 0.47 | 0.00 | 0.00 | 0.01 |
| 2461837 | | | | | | | | | | |
| knn:2332285 | 0.70 | 0.020 | 0.005 | 0.005 | 0.81 | 0.64 | 0.49 | 0.00 | 0.00 | 0.01 |
| 2461837 | | | | | | | | | | |
| rpart:2332285 | 0.68 | 0.027 | 0.081 | 0.053 | 0.79 | 0.59 | 0.59 | 0.01 | 0.04 | 0.06 |
| 2461837 | | | | | | | | | | |
| svm:2332285 | 0.74 | 0.005 | 0.013 | 0.003 | 0.72 | 0.62 | −0.16 | 0.02 | 0.01 | 0.01 |
| 2461837 | | | | | | | | | | |
| nb:2332285 | 0.76 | 0.002 | 0.001 | 0.002 | 0.89 | 0.67 | 0.51 | 0.00 | 0.00 | 0.00 |
| 2512364 | | | | | | | | | | |
| knn:2332285 | 0.72 | 0.012 | 0.001 | 0.003 | 0.89 | 0.67 | 0.56 | 0.00 | 0.01 | 0.01 |
| 2512364 | | | | | | | | | | |
| svm:2332285 | 0.82 | 0.000 | 0.001 | 0.000 | 0.85 | 0.65 | −0.12 | 0.00 | 0.00 | 0.00 |
| 2512364 | | | | | | | | | | |
| rf:2332285 | 0.76 | 0.003 | 0.002 | 0.001 | 0.85 | 0.65 | 0.49 | 0.00 | 0.00 | 0.00 |
| 2364325 | | | | | | | | | | |
| nb:2332285 | 0.78 | 0.001 | 0.001 | 0.001 | 0.89 | 0.67 | 0.50 | 0.00 | 0.00 | 0.00 |
| 2364325 | | | | | | | | | | |
| knn:2332285 | 0.72 | 0.013 | 0.006 | 0.003 | 0.82 | 0.67 | 0.48 | 0.00 | 0.00 | 0.01 |
| 2364325 | | | | | | | | | | |
| rpart:2332285 | 0.74 | 0.001 | 0.011 | 0.001 | 0.79 | 0.68 | 0.51 | 0.00 | 0.00 | 0.00 |
| 2364325 | | | | | | | | | | |
| svm:2332285 | 0.81 | 0.000 | 0.001 | 0.000 | 0.89 | 0.67 | 0.02 | 0.00 | 0.00 | 0.00 |
| 2364325 | | | | | | | | | | |
| nb:2332285 | 0.75 | 0.004 | 0.003 | 0.003 | 0.84 | 0.63 | 0.49 | 0.00 | 0.00 | 0.01 |
| 3066770 | | | | | | | | | | |
| knn:2332285 | 0.72 | 0.012 | 0.012 | 0.003 | 0.83 | 0.61 | 0.52 | 0.00 | 0.00 | 0.01 |
| 3066770 | | | | | | | | | | |
| svm:2332285 | 0.81 | 0.000 | 0.001 | 0.001 | 0.81 | 0.64 | −0.18 | 0.00 | 0.00 | 0.00 |
| 3066770 | | | | | | | | | | |
| nb:2332285 | 0.74 | 0.006 | 0.009 | 0.008 | 0.82 | 0.59 | 0.50 | 0.01 | 0.01 | 0.01 |
| 2956442 | | | | | | | | | | |
| knn:2332285 | 0.71 | 0.012 | 0.008 | 0.011 | 0.82 | 0.59 | 0.53 | 0.01 | 0.02 | 0.02 |
| 2956442 | | | | | | | | | | |
| svm:2332285 | 0.78 | 0.001 | 0.001 | 0.001 | 0.80 | 0.62 | −0.16 | 0.01 | 0.00 | 0.00 |
| 2956442 | | | | | | | | | | |
| rf:2332285 | 0.80 | 0.001 | 0.001 | 0.000 | 0.74 | 0.68 | 0.39 | 0.01 | 0.00 | 0.00 |
| 3005357 | | | | | | | | | | |
| nb:2332285 | 0.80 | 0.000 | 0.001 | 0.001 | 0.84 | 0.63 | 0.48 | 0.00 | 0.00 | 0.00 |
| 3005357 | | | | | | | | | | |
| knn:2332285 | 0.71 | 0.013 | 0.050 | 0.010 | 0.77 | 0.63 | 0.50 | 0.01 | 0.02 | 0.02 |
| 3005357 | | | | | | | | | | |
| rpart:2332285 | 0.74 | 0.003 | 0.038 | 0.006 | 0.79 | 0.59 | 0.44 | 0.00 | 0.00 | 0.01 |
| 3005357 | | | | | | | | | | |
| svm:2332285 | 0.83 | 0.000 | 0.000 | 0.000 | 0.85 | 0.65 | −0.12 | 0.00 | 0.00 | 0.00 |
| 3005357 | | | | | | | | | | |
| rf:2332285 | 0.75 | 0.003 | 0.008 | 0.002 | 0.88 | 0.62 | 0.49 | 0.00 | 0.00 | 0.01 |
| 2651515 | | | | | | | | | | |
| nb:2332285 | 0.78 | 0.001 | 0.001 | 0.003 | 0.87 | 0.58 | 0.51 | 0.01 | 0.01 | 0.01 |
| 2651515 | | | | | | | | | | |
| knn:2332285 | 0.70 | 0.021 | 0.008 | 0.002 | 0.88 | 0.60 | 0.53 | 0.00 | 0.01 | 0.01 |
| 2651515 | | | | | | | | | | |
| rpart:2332285 | 0.66 | 0.018 | 0.189 | 0.015 | 0.85 | 0.55 | 0.56 | 0.01 | 0.02 | 0.03 |
| 2651515 | | | | | | | | | | |
| svm:2332285 | 0.72 | 0.010 | 0.016 | 0.004 | 0.76 | 0.55 | −0.11 | 0.05 | 0.01 | 0.01 |
| 2651515 | | | | | | | | | | |
| rf:2478155 | 0.71 | 0.014 | 0.011 | 0.002 | 0.69 | 0.65 | 0.55 | 0.04 | 0.01 | 0.01 |
| 3139092 | | | | | | | | | | |
| nb:2478155 | 0.78 | 0.001 | 0.007 | 0.001 | 0.81 | 0.64 | 0.49 | 0.00 | 0.00 | 0.00 |
| 3139092 | | | | | | | | | | |
| knn:2478155 | 0.77 | 0.002 | 0.014 | 0.001 | 0.75 | 0.64 | 0.50 | 0.01 | 0.01 | 0.00 |
| 3139092 | | | | | | | | | | |
| rpart:2478155 | 0.72 | 0.004 | 0.030 | 0.003 | 0.76 | 0.67 | 0.49 | 0.00 | 0.01 | 0.01 |
| 3139092 | | | | | | | | | | |
| svm:2478155 | 0.71 | 0.017 | 0.010 | 0.006 | 0.65 | 0.50 | 0.54 | 0.50 | 0.02 | 0.01 |
| 3139092 | | | | | | | | | | |
| rf:2478155 | 0.76 | 0.003 | 0.035 | 0.003 | 0.74 | 0.56 | 0.59 | 0.04 | 0.01 | 0.01 |
| 2789391 | | | | | | | | | | |
| nb:2478155 | 0.74 | 0.005 | 0.030 | 0.004 | 0.80 | 0.55 | 0.50 | 0.02 | 0.00 | 0.01 |
| 2789391 | | | | | | | | | | |
| knn:2478155 | 0.69 | 0.024 | 0.125 | 0.019 | 0.80 | 0.55 | 0.54 | 0.02 | 0.02 | 0.03 |
| 2789391 | | | | | | | | | | |
| svm:2478155 | 0.69 | 0.025 | 0.030 | 0.011 | 0.74 | 0.56 | −0.06 | 0.04 | 0.01 | 0.02 |
| 2789391 | | | | | | | | | | |
| rf:2478155 | 0.74 | 0.007 | 0.014 | 0.003 | 0.80 | 0.62 | 0.67 | 0.01 | 0.02 | 0.01 |
| 3463598 | | | | | | | | | | |
| nb:2478155 | 0.75 | 0.003 | 0.005 | 0.001 | 0.78 | 0.57 | 0.52 | 0.04 | 0.00 | 0.00 |
| 3463598 | | | | | | | | | | |
| knn:2478155 | 0.71 | 0.014 | 0.047 | 0.014 | 0.66 | 0.73 | 0.44 | 0.09 | 0.07 | 0.02 |
| 3463598 | | | | | | | | | | |
| rpart:2478155 | 0.71 | 0.006 | 0.040 | 0.004 | 0.80 | 0.62 | 0.53 | 0.01 | 0.01 | 0.01 |
| 3463598 | | | | | | | | | | |
| nb:2478155 | 0.76 | 0.002 | 0.000 | 0.002 | 0.80 | 0.62 | 0.49 | 0.00 | 0.00 | 0.01 |
| 2461837 | | | | | | | | | | |
| knn:2478155 | 0.68 | 0.040 | 0.071 | 0.008 | 0.69 | 0.82 | 0.42 | 0.02 | 0.03 | 0.01 |
| 2461837 | | | | | | | | | | |
| rpart:2478155 | 0.68 | 0.019 | 0.118 | 0.017 | 0.74 | 0.61 | 0.47 | 0.01 | 0.02 | 0.02 |
| 2461837 | | | | | | | | | | |
| nb:2478155 | 0.77 | 0.001 | 0.001 | 0.001 | 0.78 | 0.65 | 0.50 | 0.01 | 0.00 | 0.00 |
| 2512364 | | | | | | | | | | |
| knn:2478155 | 0.77 | 0.002 | 0.005 | 0.001 | 0.79 | 0.68 | 0.50 | 0.00 | 0.01 | 0.00 |
| 2512364 | | | | | | | | | | |
| rf:2478155 | 0.80 | 0.001 | 0.000 | 0.000 | 0.70 | 0.77 | 0.49 | 0.02 | 0.00 | 0.00 |
| 2364325 | | | | | | | | | | |
| nb:2478155 | 0.81 | 0.000 | 0.000 | 0.000 | 0.85 | 0.65 | 0.49 | 0.00 | 0.00 | 0.00 |
| 2364325 | | | | | | | | | | |
| knn:2478155 | 0.85 | 0.000 | 0.002 | 0.000 | 0.84 | 0.63 | 0.55 | 0.00 | 0.00 | 0.00 |
| 2364325 | | | | | | | | | | |
| svm:2478155 | 0.81 | 0.000 | 0.000 | 0.000 | 0.79 | 0.68 | 0.27 | 0.00 | 0.00 | 0.00 |
| 2364325 | | | | | | | | | | |
| rf:2478155 | 0.69 | 0.030 | 0.086 | 0.015 | 0.67 | 0.69 | 0.37 | 0.05 | 0.05 | 0.02 |
| 3066770 | | | | | | | | | | |
| nb:2478155 | 0.76 | 0.002 | 0.002 | 0.002 | 0.84 | 0.63 | 0.49 | 0.00 | 0.00 | 0.01 |
| 3066770 | | | | | | | | | | |
| knn:2478155 | 0.70 | 0.020 | 0.223 | 0.010 | 0.67 | 0.55 | 0.50 | 0.17 | 0.04 | 0.02 |
| 3066770 | | | | | | | | | | |
| rpart:2478155 | 0.69 | 0.004 | 0.086 | 0.003 | 0.69 | 0.82 | 0.47 | 0.02 | 0.03 | 0.01 |
| 3066770 | | | | | | | | | | |
| rf:2478155 | 0.70 | 0.023 | 0.058 | 0.015 | 0.69 | 0.65 | 0.47 | 0.04 | 0.05 | 0.02 |
| 2956442 | | | | | | | | | | |
| nb:2478155 | 0.71 | 0.017 | 0.059 | 0.008 | 0.81 | 0.57 | 0.52 | 0.02 | 0.01 | 0.01 |
| 2956442 | | | | | | | | | | |
| knn:2478155 | 0.75 | 0.004 | 0.075 | 0.003 | 0.78 | 0.57 | 0.52 | 0.03 | 0.01 | 0.01 |
| 2956442 | | | | | | | | | | |
| rpart:2478155 | 0.69 | 0.004 | 0.086 | 0.003 | 0.69 | 0.82 | 0.47 | 0.02 | 0.03 | 0.01 |
| 2956442 | | | | | | | | | | |
| svm:2478155 | 0.68 | 0.040 | 0.047 | 0.029 | 0.74 | 0.56 | 0.09 | 0.08 | 0.05 | 0.03 |
| 2956442 | | | | | | | | | | |
| rf:2478155 | 0.75 | 0.004 | 0.011 | 0.001 | 0.76 | 0.67 | 0.66 | 0.01 | 0.01 | 0.00 |
| 3005357 | | | | | | | | | | |
| nb:2478155 | 0.80 | 0.000 | 0.002 | 0.000 | 0.79 | 0.68 | 0.44 | 0.00 | 0.00 | 0.00 |
| 3005357 | | | | | | | | | | |
| knn:2478155 | 0.80 | 0.001 | 0.019 | 0.000 | 0.69 | 0.71 | 0.41 | 0.02 | 0.00 | 0.00 |
| 3005357 | | | | | | | | | | |
| rpart:2478155 | 0.71 | 0.005 | 0.038 | 0.003 | 0.74 | 0.68 | 0.44 | 0.01 | 0.01 | 0.01 |
| 3005357 | | | | | | | | | | |
| svm:2478155 | 0.79 | 0.001 | 0.002 | 0.000 | 0.79 | 0.68 | −0.07 | 0.00 | 0.00 | 0.00 |
| 3005357 | | | | | | | | | | |
| rf:2478155 | 0.68 | 0.035 | 0.189 | 0.029 | 0.83 | 0.53 | 0.66 | 0.04 | 0.05 | 0.04 |
| 2651515 | | | | | | | | | | |
| nb:2478155 | 0.75 | 0.004 | 0.022 | 0.003 | 0.92 | 0.58 | 0.51 | 0.01 | 0.01 | 0.01 |
| 2651515 | | | | | | | | | | |
| knn:2478155 | 0.71 | 0.013 | 0.004 | 0.003 | 0.88 | 0.62 | 0.52 | 0.00 | 0.01 | 0.01 |
| 2651515 | | | | | | | | | | |
| rpart:2478155 | 0.66 | 0.018 | 0.189 | 0.015 | 0.85 | 0.55 | 0.56 | 0.01 | 0.02 | 0.03 |
| 2651515 | | | | | | | | | | |
| svm:2478155 | 0.68 | 0.033 | 0.030 | 0.033 | 0.63 | 0.48 | −0.29 | 0.63 | 0.07 | 0.04 |
| 2651515 | | | | | | | | | | |
| rf:3139092 | 0.73 | 0.010 | 0.014 | 0.007 | 0.78 | 0.57 | 0.57 | 0.02 | 0.01 | 0.01 |
| 2789391 | | | | | | | | | | |
| nb:3139092 | 0.73 | 0.008 | 0.013 | 0.007 | 0.76 | 0.60 | 0.45 | 0.01 | 0.00 | 0.01 |
| 2789391 | | | | | | | | | | |
| knn:3139092 | 0.72 | 0.010 | 0.032 | 0.004 | 0.71 | 0.59 | 0.44 | 0.04 | 0.01 | 0.01 |
| 2789391 | | | | | | | | | | |
| rpart:3139092 | 0.72 | 0.004 | 0.030 | 0.003 | 0.76 | 0.67 | 0.49 | 0.00 | 0.01 | 0.01 |
| 2789391 | | | | | | | | | | |
| rf:3139092 | 0.72 | 0.010 | 0.014 | 0.005 | 0.70 | 0.69 | 0.43 | 0.02 | 0.02 | 0.01 |
| 3463598 | | | | | | | | | | |
| nb:3139092 | 0.73 | 0.006 | 0.007 | 0.005 | 0.77 | 0.63 | 0.48 | 0.01 | 0.01 | 0.01 |
| 3463598 | | | | | | | | | | |
| knn:3139092 | 0.75 | 0.004 | 0.014 | 0.002 | 0.77 | 0.70 | 0.47 | 0.00 | 0.01 | 0.00 |
| 3463598 | | | | | | | | | | |
| rpart:3139092 | 0.71 | 0.006 | 0.040 | 0.004 | 0.80 | 0.62 | 0.53 | 0.01 | 0.01 | 0.01 |
| 3463598 | | | | | | | | | | |
| svm:3139092 | 0.72 | 0.012 | 0.014 | 0.036 | 0.67 | 0.69 | −0.14 | 0.04 | 0.05 | 0.04 |
| 3463598 | | | | | | | | | | |
| nb:3139092 | 0.73 | 0.008 | 0.003 | 0.006 | 0.76 | 0.60 | 0.46 | 0.01 | 0.01 | 0.01 |
| 2461837 | | | | | | | | | | |
| rpart:3139092 | 0.68 | 0.019 | 0.118 | 0.017 | 0.74 | 0.61 | 0.47 | 0.01 | 0.02 | 0.02 |
| 2461837 | | | | | | | | | | |
| svm:3139092 | 0.68 | 0.035 | 0.022 | 0.028 | 0.76 | 0.60 | 0.03 | 0.02 | 0.04 | 0.03 |
| 2461837 | | | | | | | | | | |
| nb:3139092 | 0.76 | 0.002 | 0.003 | 0.002 | 0.76 | 0.60 | 0.48 | 0.01 | 0.00 | 0.00 |
| 2512364 | | | | | | | | | | |
| rf:3139092 | 0.72 | 0.012 | 0.014 | 0.007 | 0.74 | 0.61 | 0.54 | 0.04 | 0.02 | 0.01 |
| 2364325 | | | | | | | | | | |
| nb:3139092 | 0.80 | 0.000 | 0.001 | 0.001 | 0.83 | 0.70 | 0.45 | 0.00 | 0.00 | 0.00 |
| 2364325 | | | | | | | | | | |
| knn:3139092 | 0.76 | 0.002 | 0.001 | 0.001 | 0.81 | 0.64 | 0.52 | 0.00 | 0.00 | 0.00 |
| 2364325 | | | | | | | | | | |
| rpart:3139092 | 0.72 | 0.004 | 0.030 | 0.003 | 0.76 | 0.67 | 0.49 | 0.00 | 0.01 | 0.01 |
| 2364325 | | | | | | | | | | |
| svm:3139092 | 0.80 | 0.000 | 0.001 | 0.000 | 0.80 | 0.71 | −0.15 | 0.00 | 0.00 | 0.00 |
| 2364325 | | | | | | | | | | |
| nb:3139092 | 0.73 | 0.009 | 0.009 | 0.008 | 0.73 | 0.58 | 0.46 | 0.03 | 0.01 | 0.01 |
| 3066770 | | | | | | | | | | |
| knn:3139092 | 0.74 | 0.006 | 0.006 | 0.004 | 0.73 | 0.58 | 0.51 | 0.03 | 0.01 | 0.01 |
| 3066770 | | | | | | | | | | |
| rpart:3139092 | 0.72 | 0.004 | 0.030 | 0.003 | 0.76 | 0.67 | 0.49 | 0.00 | 0.01 | 0.01 |
| 3066770 | | | | | | | | | | |
| svm:3139092 | 0.73 | 0.008 | 0.003 | 0.007 | 0.75 | 0.64 | 0.01 | 0.01 | 0.01 | 0.01 |
| 3066770 | | | | | | | | | | |
| rf:3139092 | 0.67 | 0.049 | 0.030 | 0.032 | 0.76 | 0.67 | 0.50 | 0.00 | 0.06 | 0.04 |
| 2956442 | | | | | | | | | | |
| nb:3139092 | 0.71 | 0.016 | 0.020 | 0.018 | 0.74 | 0.61 | 0.45 | 0.03 | 0.02 | 0.02 |
| 2956442 | | | | | | | | | | |
| knn:3139092 | 0.74 | 0.005 | 0.005 | 0.003 | 0.75 | 0.72 | 0.44 | 0.00 | 0.01 | 0.01 |
| 2956442 | | | | | | | | | | |
| rpart:3139092 | 0.72 | 0.004 | 0.030 | 0.003 | 0.76 | 0.67 | 0.49 | 0.00 | 0.01 | 0.01 |
| 2956442 | | | | | | | | | | |
| svm:3139092 | 0.75 | 0.004 | 0.003 | 0.006 | 0.76 | 0.67 | −0.02 | 0.00 | 0.01 | 0.01 |
| 2956442 | | | | | | | | | | |
| rf:3139092 | 0.71 | 0.015 | 0.013 | 0.001 | 0.74 | 0.61 | 0.56 | 0.01 | 0.00 | 0.00 |
| 3005357 | | | | | | | | | | |
| nb:3139092 | 0.77 | 0.002 | 0.000 | 0.001 | 0.78 | 0.74 | 0.44 | 0.00 | 0.00 | 0.00 |
| 3005357 | | | | | | | | | | |
| knn:3139092 | 0.73 | 0.007 | 0.001 | 0.001 | 0.77 | 0.87 | 0.43 | 0.00 | 0.00 | 0.00 |
| 3005357 | | | | | | | | | | |
| rpart:3139092 | 0.71 | 0.005 | 0.038 | 0.003 | 0.74 | 0.68 | 0.44 | 0.01 | 0.01 | 0.01 |
| 3005357 | | | | | | | | | | |
| svm:3139092 | 0.75 | 0.004 | 0.000 | 0.002 | 0.76 | 0.67 | 0.14 | 0.00 | 0.00 | 0.00 |
| 3005357 | | | | | | | | | | |
| nb:3139092 | 0.69 | 0.028 | 0.022 | 0.009 | 0.86 | 0.56 | 0.50 | 0.01 | 0.01 | 0.02 |
| 2651515 | | | | | | | | | | |
| rpart:3139092 | 0.68 | 0.031 | 0.189 | 0.032 | 0.68 | 0.57 | 0.45 | 0.06 | 0.03 | 0.04 |
| 2651515 | | | | | | | | | | |
| rf:2789391 | 0.73 | 0.008 | 0.019 | 0.005 | 0.79 | 0.59 | 0.50 | 0.01 | 0.01 | 0.01 |
| 3463598 | | | | | | | | | | |
| nb:2789391 | 0.71 | 0.013 | 0.005 | 0.010 | 0.79 | 0.59 | 0.49 | 0.01 | 0.01 | 0.02 |
| 3463598 | | | | | | | | | | |
| knn:2789391 | 0.71 | 0.014 | 0.032 | 0.009 | 0.83 | 0.61 | 0.54 | 0.00 | 0.02 | 0.01 |
| 3463598 | | | | | | | | | | |
| rpart:2789391 | 0.71 | 0.006 | 0.040 | 0.004 | 0.80 | 0.62 | 0.53 | 0.01 | 0.01 | 0.01 |
| 3463598 | | | | | | | | | | |
| svm:2789391 | 0.72 | 0.012 | 0.005 | 0.008 | 0.81 | 0.64 | −0.07 | 0.00 | 0.01 | 0.01 |
| 3463598 | | | | | | | | | | |
| rf:2789391 | 0.72 | 0.012 | 0.030 | 0.012 | 0.73 | 0.58 | 0.47 | 0.03 | 0.01 | 0.02 |
| 2461837 | | | | | | | | | | |
| nb:2789391 | 0.69 | 0.028 | 0.007 | 0.015 | 0.78 | 0.65 | 0.42 | 0.00 | 0.01 | 0.02 |
| 2461837 | | | | | | | | | | |
| knn:2789391 | 0.74 | 0.005 | 0.081 | 0.004 | 0.79 | 0.59 | 0.50 | 0.01 | 0.00 | 0.01 |
| 2461837 | | | | | | | | | | |
| rpart:2789391 | 0.68 | 0.019 | 0.118 | 0.017 | 0.74 | 0.61 | 0.47 | 0.01 | 0.02 | 0.02 |
| 2461837 | | | | | | | | | | |
| rf:2789391 | 0.71 | 0.015 | 0.050 | 0.010 | 0.77 | 0.63 | 0.45 | 0.00 | 0.01 | 0.01 |
| 2512364 | | | | | | | | | | |
| nb:2789391 | 0.72 | 0.010 | 0.005 | 0.007 | 0.75 | 0.64 | 0.44 | 0.01 | 0.01 | 0.01 |
| 2512364 | | | | | | | | | | |
| svm:2789391 | 0.72 | 0.012 | 0.036 | 0.031 | 0.74 | 0.56 | −0.31 | 0.03 | 0.04 | 0.04 |
| 2512364 | | | | | | | | | | |
| rf:2789391 | 0.74 | 0.005 | 0.047 | 0.006 | 0.71 | 0.56 | 0.45 | 0.04 | 0.01 | 0.01 |
| 2364325 | | | | | | | | | | |
| nb:2789391 | 0.74 | 0.004 | 0.005 | 0.003 | 0.78 | 0.65 | 0.43 | 0.00 | 0.00 | 0.01 |
| 2364325 | | | | | | | | | | |
| knn:2789391 | 0.79 | 0.001 | 0.019 | 0.001 | 0.88 | 0.60 | 0.49 | 0.00 | 0.00 | 0.00 |
| 2364325 | | | | | | | | | | |
| svm:2789391 | 0.73 | 0.007 | 0.005 | 0.005 | 0.78 | 0.65 | −0.26 | 0.00 | 0.00 | 0.01 |
| 2364325 | | | | | | | | | | |
| rf:2789391 | 0.70 | 0.021 | 0.012 | 0.014 | 0.76 | 0.60 | 0.51 | 0.02 | 0.01 | 0.02 |
| 3066770 | | | | | | | | | | |
| nb:2789391 | 0.69 | 0.030 | 0.004 | 0.017 | 0.87 | 0.58 | 0.50 | 0.00 | 0.01 | 0.02 |
| 3066770 | | | | | | | | | | |
| knn:2789391 | 0.78 | 0.001 | 0.005 | 0.001 | 0.88 | 0.62 | 0.46 | 0.00 | 0.00 | 0.00 |
| 3066770 | | | | | | | | | | |
| svm:2789391 | 0.70 | 0.022 | 0.003 | 0.012 | 0.89 | 0.64 | −0.01 | 0.00 | 0.01 | 0.02 |
| 3066770 | | | | | | | | | | |
| rf:2789391 | 0.69 | 0.027 | 0.098 | 0.021 | 0.74 | 0.56 | 0.48 | 0.04 | 0.03 | 0.03 |
| 2956442 | | | | | | | | | | |
| nb:2789391 | 0.68 | 0.040 | 0.047 | 0.032 | 0.78 | 0.57 | 0.49 | 0.02 | 0.03 | 0.04 |
| 2956442 | | | | | | | | | | |
| knn:2789391 | 0.67 | 0.050 | 0.118 | 0.027 | 0.80 | 0.55 | 0.54 | 0.02 | 0.02 | 0.04 |
| 2956442 | | | | | | | | | | |
| rf:2789391 | 0.75 | 0.004 | 0.008 | 0.002 | 0.75 | 0.72 | 0.34 | 0.00 | 0.00 | 0.00 |
| 3005357 | | | | | | | | | | |
| nb:2789391 | 0.74 | 0.006 | 0.011 | 0.005 | 0.77 | 0.63 | 0.44 | 0.00 | 0.00 | 0.01 |
| 3005357 | | | | | | | | | | |
| svm:2789391 | 0.70 | 0.023 | 0.016 | 0.032 | 0.78 | 0.57 | −0.24 | 0.02 | 0.06 | 0.05 |
| 3005357 | | | | | | | | | | |
| rf:2789391 | 0.75 | 0.005 | 0.081 | 0.005 | 0.86 | 0.56 | 0.56 | 0.01 | 0.01 | 0.01 |
| 2651515 | | | | | | | | | | |
| nb:2789391 | 0.70 | 0.021 | 0.013 | 0.015 | 0.85 | 0.55 | 0.49 | 0.03 | 0.02 | 0.03 |
| 2651515 | | | | | | | | | | |
| knn:2789391 | 0.73 | 0.007 | 0.026 | 0.007 | 0.88 | 0.60 | 0.48 | 0.00 | 0.02 | 0.01 |
| 2651515 | | | | | | | | | | |
| rpart:2789391 | 0.66 | 0.018 | 0.189 | 0.015 | 0.85 | 0.55 | 0.56 | 0.01 | 0.02 | 0.03 |
| 2651515 | | | | | | | | | | |
| svm:2789391 | 0.69 | 0.026 | 0.013 | 0.019 | 0.79 | 0.59 | −0.32 | 0.02 | 0.02 | 0.03 |
| 2651515 | | | | | | | | | | |
| rf:3463598 | 0.68 | 0.034 | 0.092 | 0.029 | 0.71 | 0.67 | 0.47 | 0.02 | 0.05 | 0.03 |
| 2461837 | | | | | | | | | | |
| nb:3463598 | 0.71 | 0.014 | 0.020 | 0.014 | 0.76 | 0.60 | 0.47 | 0.01 | 0.01 | 0.02 |
| 2461837 | | | | | | | | | | |
| rpart:3463598 | 0.68 | 0.019 | 0.118 | 0.017 | 0.74 | 0.61 | 0.47 | 0.01 | 0.02 | 0.02 |
| 2461837 | | | | | | | | | | |
| svm:3463598 | 0.69 | 0.030 | 0.059 | 0.015 | 0.74 | 0.61 | 0.12 | 0.02 | 0.07 | 0.02 |
| 2461837 | | | | | | | | | | |
| rf:3463598 | 0.68 | 0.034 | 0.104 | 0.017 | 0.81 | 0.57 | 0.61 | 0.03 | 0.04 | 0.02 |
| 2512364 | | | | | | | | | | |
| nb:3463598 | 0.74 | 0.005 | 0.001 | 0.004 | 0.81 | 0.64 | 0.51 | 0.00 | 0.01 | 0.01 |
| 2512364 | | | | | | | | | | |
| knn:3463598 | 0.72 | 0.010 | 0.011 | 0.009 | 0.85 | 0.55 | 0.61 | 0.05 | 0.03 | 0.02 |
| 2512364 | | | | | | | | | | |
| rpart:3463598 | 0.71 | 0.006 | 0.040 | 0.004 | 0.80 | 0.62 | 0.53 | 0.01 | 0.01 | 0.01 |
| 2512364 | | | | | | | | | | |
| svm:3463598 | 0.69 | 0.032 | 0.004 | 0.007 | 0.85 | 0.65 | 0.07 | 0.00 | 0.02 | 0.01 |
| 2512364 | | | | | | | | | | |
| rf:3463598 | 0.75 | 0.004 | 0.005 | 0.001 | 0.85 | 0.65 | 0.57 | 0.00 | 0.01 | 0.00 |
| 2364325 | | | | | | | | | | |
| nb:3463598 | 0.75 | 0.004 | 0.001 | 0.001 | 0.89 | 0.67 | 0.51 | 0.00 | 0.00 | 0.00 |
| 2364325 | | | | | | | | | | |
| knn:3463598 | 0.79 | 0.001 | 0.001 | 0.000 | 0.88 | 0.62 | 0.53 | 0.00 | 0.00 | 0.00 |
| 2364325 | | | | | | | | | | |
| rpart:3463598 | 0.71 | 0.006 | 0.040 | 0.004 | 0.80 | 0.62 | 0.53 | 0.01 | 0.01 | 0.01 |
| 2364325 | | | | | | | | | | |
| svm:3463598 | 0.70 | 0.020 | 0.001 | 0.003 | 0.89 | 0.67 | 0.04 | 0.00 | 0.01 | 0.01 |
| 2364325 | | | | | | | | | | |
| rf:3463598 | 0.68 | 0.040 | 0.011 | 0.019 | 0.79 | 0.59 | 0.55 | 0.02 | 0.04 | 0.02 |
| 3066770 | | | | | | | | | | |
| nb:3463598 | 0.71 | 0.016 | 0.003 | 0.010 | 0.84 | 0.63 | 0.50 | 0.00 | 0.01 | 0.02 |
| 3066770 | | | | | | | | | | |
| knn:3463598 | 0.76 | 0.002 | 0.005 | 0.001 | 0.89 | 0.64 | 0.54 | 0.00 | 0.00 | 0.00 |
| 3066770 | | | | | | | | | | |
| rpart:3463598 | 0.71 | 0.006 | 0.040 | 0.004 | 0.80 | 0.62 | 0.53 | 0.01 | 0.01 | 0.01 |
| 3066770 | | | | | | | | | | |
| svm:3463598 | 0.72 | 0.012 | 0.003 | 0.006 | 0.85 | 0.65 | −0.29 | 0.00 | 0.00 | 0.01 |
| 3066770 | | | | | | | | | | |
| rf:3463598 | 0.70 | 0.019 | 0.024 | 0.011 | 0.70 | 0.63 | 0.44 | 0.03 | 0.03 | 0.02 |
| 2956442 | | | | | | | | | | |
| nb:3463598 | 0.69 | 0.030 | 0.028 | 0.022 | 0.76 | 0.60 | 0.49 | 0.02 | 0.03 | 0.03 |
| 2956442 | | | | | | | | | | |
| knn:3463598 | 0.71 | 0.016 | 0.054 | 0.004 | 0.82 | 0.59 | 0.53 | 0.01 | 0.01 | 0.01 |
| 2956442 | | | | | | | | | | |
| svm:3463598 | 0.70 | 0.023 | 0.036 | 0.010 | 0.79 | 0.59 | −0.11 | 0.01 | 0.02 | 0.02 |
| 2956442 | | | | | | | | | | |
| rf:3463598 | 0.74 | 0.006 | 0.008 | 0.002 | 0.75 | 0.64 | 0.48 | 0.01 | 0.01 | 0.01 |
| 3005357 | | | | | | | | | | |
| nb:3463598 | 0.76 | 0.002 | 0.009 | 0.002 | 0.79 | 0.59 | 0.50 | 0.01 | 0.00 | 0.01 |
| 3005357 | | | | | | | | | | |
| rpart:3463598 | 0.71 | 0.006 | 0.040 | 0.004 | 0.80 | 0.62 | 0.53 | 0.01 | 0.01 | 0.01 |
| 3005357 | | | | | | | | | | |
| rf:3463598 | 0.69 | 0.027 | 0.019 | 0.004 | 0.80 | 0.55 | 0.54 | 0.04 | 0.01 | 0.01 |
| 2651515 | | | | | | | | | | |
| nb:3463598 | 0.69 | 0.028 | 0.010 | 0.013 | 0.80 | 0.55 | 0.50 | 0.05 | 0.03 | 0.02 |
| 2651515 | | | | | | | | | | |
| knn:3463598 | 0.69 | 0.023 | 0.304 | 0.025 | 0.69 | 0.48 | 0.70 | 0.35 | 0.07 | 0.03 |
| 2651515 | | | | | | | | | | |
| rpart:3463598 | 0.66 | 0.018 | 0.189 | 0.015 | 0.85 | 0.55 | 0.56 | 0.01 | 0.02 | 0.03 |
| 2651515 | | | | | | | | | | |
| nb:2461837 | 0.73 | 0.008 | 0.016 | 0.005 | 0.78 | 0.65 | 0.46 | 0.00 | 0.01 | 0.01 |
| 2512364 | | | | | | | | | | |
| knn:2461837 | 0.72 | 0.010 | 0.008 | 0.009 | 0.78 | 0.65 | 0.50 | 0.00 | 0.01 | 0.01 |
| 2512364 | | | | | | | | | | |
| rf:2461837 | 0.74 | 0.005 | 0.015 | 0.002 | 0.84 | 0.63 | 0.54 | 0.00 | 0.00 | 0.00 |
| 2364325 | | | | | | | | | | |
| nb:2461837 | 0.77 | 0.001 | 0.001 | 0.002 | 0.84 | 0.63 | 0.49 | 0.00 | 0.00 | 0.00 |
| 2364325 | | | | | | | | | | |
| knn:2461837 | 0.73 | 0.007 | 0.024 | 0.006 | 0.83 | 0.61 | 0.59 | 0.00 | 0.01 | 0.01 |
| 2364325 | | | | | | | | | | |
| rpart:2461837 | 0.68 | 0.019 | 0.118 | 0.017 | 0.74 | 0.61 | 0.47 | 0.01 | 0.02 | 0.02 |
| 2364325 | | | | | | | | | | |
| svm:2461837 | 0.71 | 0.015 | 0.002 | 0.009 | 0.83 | 0.70 | −0.20 | 0.00 | 0.01 | 0.01 |
| 2364325 | | | | | | | | | | |
| nb:2461837 | 0.70 | 0.023 | 0.026 | 0.015 | 0.78 | 0.57 | 0.49 | 0.01 | 0.01 | 0.02 |
| 3066770 | | | | | | | | | | |
| svm:2461837 | 0.70 | 0.023 | 0.018 | 0.062 | 0.67 | 0.50 | 0.15 | 0.25 | 0.06 | 0.08 |
| 3066770 | | | | | | | | | | |
| nb:2461837 | 0.67 | 0.047 | 0.036 | 0.031 | 0.77 | 0.63 | 0.43 | 0.01 | 0.03 | 0.04 |
| 2956442 | | | | | | | | | | |
| knn:2461837 | 0.70 | 0.020 | 0.024 | 0.019 | 0.72 | 0.54 | 0.53 | 0.07 | 0.02 | 0.02 |
| 2956442 | | | | | | | | | | |
| rpart:2461837 | 0.68 | 0.019 | 0.118 | 0.017 | 0.74 | 0.61 | 0.47 | 0.01 | 0.02 | 0.02 |
| 2956442 | | | | | | | | | | |
| rf:2461837 | 0.70 | 0.024 | 0.022 | 0.008 | 0.74 | 0.61 | 0.45 | 0.01 | 0.01 | 0.01 |
| 3005357 | | | | | | | | | | |
| nb:2461837 | 0.74 | 0.005 | 0.003 | 0.007 | 0.75 | 0.58 | 0.46 | 0.01 | 0.01 | 0.01 |
| 3005357 | | | | | | | | | | |
| knn:2461837 | 0.75 | 0.003 | 0.005 | 0.001 | 0.80 | 0.71 | 0.44 | 0.00 | 0.00 | 0.00 |
| 3005357 | | | | | | | | | | |
| rpart:2461837 | 0.68 | 0.019 | 0.118 | 0.017 | 0.74 | 0.61 | 0.47 | 0.01 | 0.02 | 0.02 |
| 3005357 | | | | | | | | | | |
| svm:2461837 | 0.71 | 0.015 | 0.003 | 0.011 | 0.80 | 0.71 | −0.07 | 0.00 | 0.01 | 0.02 |
| 3005357 | | | | | | | | | | |
| rf:2461837 | 0.67 | 0.046 | 0.125 | 0.042 | 0.75 | 0.58 | 0.53 | 0.02 | 0.05 | 0.05 |
| 2651515 | | | | | | | | | | |
| nb:2461837 | 0.72 | 0.010 | 0.017 | 0.010 | 0.87 | 0.58 | 0.48 | 0.00 | 0.01 | 0.02 |
| 2651515 | | | | | | | | | | |
| svm:2461837 | 0.68 | 0.040 | 0.176 | 0.027 | 0.68 | 0.52 | −0.30 | 0.27 | 0.04 | 0.04 |
| 2651515 | | | | | | | | | | |
| nb:2512364 | 0.76 | 0.002 | 0.000 | 0.001 | 0.79 | 0.68 | 0.44 | 0.00 | 0.00 | 0.00 |
| 2364325 | | | | | | | | | | |
| rpart:2512364 | 0.69 | 0.011 | 0.075 | 0.009 | 0.73 | 0.65 | 0.48 | 0.02 | 0.02 | 0.01 |
| 2364325 | | | | | | | | | | |
| svm:2512364 | 0.74 | 0.005 | 0.000 | 0.001 | 0.83 | 0.70 | −0.20 | 0.00 | 0.00 | 0.00 |
| 2364325 | | | | | | | | | | |
| nb:2512364 | 0.74 | 0.004 | 0.003 | 0.004 | 0.77 | 0.63 | 0.47 | 0.01 | 0.01 | 0.01 |
| 3066770 | | | | | | | | | | |
| knn:2512364 | 0.75 | 0.004 | 0.026 | 0.002 | 0.80 | 0.62 | 0.53 | 0.01 | 0.00 | 0.01 |
| 3066770 | | | | | | | | | | |
| svm:2512364 | 0.72 | 0.012 | 0.013 | 0.014 | 0.75 | 0.64 | −0.01 | 0.02 | 0.02 | 0.02 |
| 3066770 | | | | | | | | | | |
| nb:2512364 | 0.71 | 0.016 | 0.047 | 0.012 | 0.79 | 0.59 | 0.50 | 0.01 | 0.02 | 0.02 |
| 2956442 | | | | | | | | | | |
| svm:2512364 | 0.70 | 0.021 | 0.047 | 0.015 | 0.79 | 0.59 | 0.04 | 0.01 | 0.02 | 0.02 |
| 2956442 | | | | | | | | | | |
| nb:2512364 | 0.76 | 0.003 | 0.005 | 0.002 | 0.81 | 0.64 | 0.50 | 0.00 | 0.00 | 0.00 |
| 3005357 | | | | | | | | | | |
| knn:2512364 | 0.67 | 0.050 | 0.335 | 0.056 | 0.67 | 0.52 | 0.50 | 0.24 | 0.07 | 0.06 |
| 3005357 | | | | | | | | | | |
| nb:2512364 | 0.75 | 0.003 | 0.005 | 0.005 | 0.69 | 0.65 | 0.39 | 0.02 | 0.01 | 0.01 |
| 2651515 | | | | | | | | | | |
| knn:2512364 | 0.68 | 0.039 | 0.178 | 0.037 | 0.75 | 0.58 | 0.50 | 0.06 | 0.06 | 0.04 |
| 2651515 | | | | | | | | | | |
| rpart:2512364 | 0.66 | 0.018 | 0.189 | 0.015 | 0.85 | 0.55 | 0.56 | 0.01 | 0.02 | 0.03 |
| 2651515 | | | | | | | | | | |
| nb:2364325 | 0.76 | 0.003 | 0.002 | 0.002 | 0.88 | 0.62 | 0.50 | 0.00 | 0.00 | 0.01 |
| 3066770 | | | | | | | | | | |
| knn:2364325 | 0.78 | 0.001 | 0.009 | 0.001 | 0.88 | 0.62 | 0.52 | 0.00 | 0.00 | 0.00 |
| 3066770 | | | | | | | | | | |
| rpart:2364325 | 0.74 | 0.001 | 0.011 | 0.001 | 0.79 | 0.68 | 0.51 | 0.00 | 0.00 | 0.00 |
| 3066770 | | | | | | | | | | |
| svm:2364325 | 0.75 | 0.004 | 0.002 | 0.001 | 0.84 | 0.63 | 0.01 | 0.00 | 0.00 | 0.00 |
| 3066770 | | | | | | | | | | |
| rf:2364325 | 0.75 | 0.004 | 0.006 | 0.003 | 0.83 | 0.61 | 0.55 | 0.00 | 0.01 | 0.01 |
| 2956442 | | | | | | | | | | |
| nb:2364325 | 0.72 | 0.010 | 0.004 | 0.006 | 0.83 | 0.61 | 0.49 | 0.00 | 0.01 | 0.01 |
| 2956442 | | | | | | | | | | |
| knn:2364325 | 0.74 | 0.006 | 0.019 | 0.004 | 0.82 | 0.59 | 0.54 | 0.01 | 0.01 | 0.01 |
| 2956442 | | | | | | | | | | |
| rpart:2364325 | 0.74 | 0.001 | 0.011 | 0.001 | 0.79 | 0.68 | 0.51 | 0.00 | 0.00 | 0.00 |
| 2956442 | | | | | | | | | | |
| svm:2364325 | 0.74 | 0.004 | 0.004 | 0.004 | 0.81 | 0.57 | 0.04 | 0.02 | 0.01 | 0.01 |
| 2956442 | | | | | | | | | | |
| rf:2364325 | 0.77 | 0.002 | 0.008 | 0.000 | 0.76 | 0.67 | 0.47 | 0.01 | 0.00 | 0.00 |
| 3005357 | | | | | | | | | | |
| nb:2364325 | 0.79 | 0.001 | 0.000 | 0.000 | 0.86 | 0.68 | 0.48 | 0.00 | 0.00 | 0.00 |
| 3005357 | | | | | | | | | | |
| knn:2364325 | 0.79 | 0.001 | 0.001 | 0.000 | 0.89 | 0.67 | 0.54 | 0.00 | 0.00 | 0.00 |
| 3005357 | | | | | | | | | | |
| rpart:2364325 | 0.70 | 0.015 | 0.038 | 0.010 | 0.74 | 0.68 | 0.37 | 0.01 | 0.02 | 0.01 |
| 3005357 | | | | | | | | | | |
| svm:2364325 | 0.78 | 0.001 | 0.000 | 0.000 | 0.86 | 0.71 | 0.10 | 0.00 | 0.00 | 0.00 |
| 3005357 | | | | | | | | | | |
| rf:2364325 | 0.74 | 0.006 | 0.050 | 0.008 | 0.86 | 0.56 | 0.58 | 0.01 | 0.01 | 0.02 |
| 2651515 | | | | | | | | | | |
| nb:2364325 | 0.77 | 0.001 | 0.001 | 0.002 | 0.89 | 0.51 | 0.56 | 0.03 | 0.01 | 0.01 |
| 2651515 | | | | | | | | | | |
| knn:2364325 | 0.71 | 0.013 | 0.275 | 0.020 | 0.90 | 0.53 | 0.61 | 0.01 | 0.03 | 0.03 |
| 2651515 | | | | | | | | | | |
| rpart:2364325 | 0.66 | 0.018 | 0.189 | 0.015 | 0.85 | 0.55 | 0.56 | 0.01 | 0.02 | 0.03 |
| 2651515 | | | | | | | | | | |
| svm:2364325 | 0.71 | 0.014 | 0.020 | 0.014 | 0.85 | 0.55 | 0.24 | 0.06 | 0.04 | 0.02 |
| 2651515 | | | | | | | | | | |
| nb:3066770 | 0.67 | 0.047 | 0.063 | 0.036 | 0.71 | 0.56 | 0.47 | 0.05 | 0.04 | 0.04 |
| 2956442 | | | | | | | | | | |
| knn:3066770 | 0.70 | 0.020 | 0.043 | 0.015 | 0.72 | 0.54 | 0.49 | 0.07 | 0.02 | 0.02 |
| 2956442 | | | | | | | | | | |
| rpart:3066770 | 0.66 | 0.028 | 0.223 | 0.025 | 0.80 | 0.55 | 0.50 | 0.02 | 0.02 | 0.03 |
| 2956442 | | | | | | | | | | |
| svm:3066770 | 0.67 | 0.047 | 0.123 | 0.036 | 0.71 | 0.52 | −0.08 | 0.13 | 0.04 | 0.04 |
| 2956442 | | | | | | | | | | |
| rf:3066770 | 0.74 | 0.006 | 0.018 | 0.002 | 0.76 | 0.60 | 0.59 | 0.01 | 0.00 | 0.01 |
| 3005357 | | | | | | | | | | |
| nb:3066770 | 0.76 | 0.003 | 0.002 | 0.004 | 0.76 | 0.55 | 0.50 | 0.01 | 0.00 | 0.01 |
| 3005357 | | | | | | | | | | |
| knn:3066770 | 0.74 | 0.005 | 0.030 | 0.006 | 0.78 | 0.57 | 0.50 | 0.01 | 0.01 | 0.01 |
| 3005357 | | | | | | | | | | |
| rpart:3066770 | 0.70 | 0.011 | 0.038 | 0.009 | 0.73 | 0.58 | 0.56 | 0.04 | 0.02 | 0.01 |
| 3005357 | | | | | | | | | | |
| svm:3066770 | 0.75 | 0.003 | 0.005 | 0.003 | 0.79 | 0.59 | 0.12 | 0.01 | 0.01 | 0.01 |
| 3005357 | | | | | | | | | | |
| nb:3066770 | 0.70 | 0.018 | 0.013 | 0.014 | 0.90 | 0.53 | 0.54 | 0.01 | 0.02 | 0.03 |
| 2651515 | | | | | | | | | | |
| rpart:3066770 | 0.66 | 0.018 | 0.189 | 0.015 | 0.85 | 0.55 | 0.56 | 0.01 | 0.02 | 0.03 |
| 2651515 | | | | | | | | | | |
| rf:2956442 | 0.72 | 0.012 | 0.018 | 0.015 | 0.71 | 0.56 | 0.46 | 0.07 | 0.03 | 0.02 |
| 3005357 | | | | | | | | | | |
| nb:2956442 | 0.71 | 0.015 | 0.028 | 0.014 | 0.79 | 0.59 | 0.48 | 0.01 | 0.02 | 0.02 |
| 3005357 | | | | | | | | | | |
| rpart:2956442 | 0.71 | 0.005 | 0.038 | 0.003 | 0.74 | 0.68 | 0.44 | 0.01 | 0.01 | 0.01 |
| 3005357 | | | | | | | | | | |
| svm:2956442 | 0.67 | 0.047 | 0.033 | 0.117 | 0.73 | 0.65 | −0.28 | 0.03 | 0.20 | 0.13 |
| 3005357 | | | | | | | | | | |
| rf:2956442 | 0.72 | 0.012 | 0.015 | 0.006 | 0.90 | 0.53 | 0.58 | 0.04 | 0.01 | 0.01 |
| 2651515 | | | | | | | | | | |
| nb:2956442 | 0.69 | 0.028 | 0.020 | 0.025 | 0.86 | 0.49 | 0.61 | 0.17 | 0.05 | 0.03 |
| 2651515 | | | | | | | | | | |
| knn:2956442 | 0.76 | 0.003 | 0.047 | 0.008 | 0.76 | 0.55 | 0.48 | 0.05 | 0.02 | 0.02 |
| 2651515 | | | | | | | | | | |
| rpart:2956442 | 0.66 | 0.018 | 0.189 | 0.015 | 0.85 | 0.55 | 0.56 | 0.01 | 0.02 | 0.03 |
| 2651515 | | | | | | | | | | |
| rf:3005357 | 0.73 | 0.009 | 0.008 | 0.006 | 0.74 | 0.61 | 0.45 | 0.03 | 0.01 | 0.01 |
| 2651515 | | | | | | | | | | |
| nb:3005357 | 0.77 | 0.002 | 0.007 | 0.002 | 0.89 | 0.51 | 0.56 | 0.03 | 0.00 | 0.01 |
| 2651515 | | | | | | | | | | |
| knn:3005357 | 0.70 | 0.019 | 0.133 | 0.022 | 0.67 | 0.50 | 0.50 | 0.35 | 0.05 | 0.03 |
| 2651515 | | | | | | | | | | |
| rpart:3005357 | 0.66 | 0.018 | 0.189 | 0.015 | 0.85 | 0.55 | 0.56 | 0.01 | 0.02 | 0.03 |
| 2651515 | | | | | | | | | | |
| svm:3005357 | 0.71 | 0.013 | 0.036 | 0.026 | 0.74 | 0.61 | −0.45 | 0.03 | 0.06 | 0.03 |
| 2651515 | | | | | | | | | | |

Performance of pairwise, threewise and fourwise combinations of the 15 markers across different metrics for the Recurrence endpoint for the subset of patients that excludes pT2 (organ confined disease) cases. All classifiers have a Wilcox P-value <=0.05

### Example 3

### A 1,425 Biomarker Library is Prognostic for Muscle Invasive Bladder Cancer Recurrence

In order to define, from the entire set of 1.4 million features available in the array, a comprehensive library of prognostic markers for the recurrence of muscle invasive bladder cancer, features were selected based on the pooled dataset (n=199) presented in Example 1 as follows. Features having an AUC greater or equal than 0.6, a Wilcox p-value less or equal than 0.05 and a MFD greater or equal than 1.25 (or MFD less or equal 0.8) were kept. Features with one or more probes deemed unreliable, unmappable or features having less than 4 probes were removed. After applying these steps, 1,440 markers remained, of which 15 corresponded to the markers already presented in Example 1 (Table 2B). The remaining markers are presented in Table 16. The markers listed in Table 16 were statistically significant for the differential expression between patients with and without bladder cancer recurrence post-cystectomy. Accordingly, Table 16 provides a biomarker library for muscle invasive bladder cancer recurrence. Gene Ontology enrichment assessment of these markers showed that they are highly enriched for multiple biological processes including translation, splicing and cell cycle progression among others processes (Table 17).

Table 18 shows the performance of 1,425 biomarkers of the present invention across various metrics for the recurrence endpoint on the pooled discovery and validation cohort (n=199). Additionally, the good performance of these markers is demonstrated within a clinically relevant set of patients, such as those with negative lymph node involvement (Table 19). Based on the multiple metrics shown in Table 18, these results demonstrate that the library of 1,440 markers of the present invention are useful for prognosing, diagnosing, and predicting the recurrence of muscle invasive bladder cancer.

In addition to the good performance of these markers as univariable predictors, pairwise combinations of the markers (pairwise classifiers) through a machine learning algorithm results in enhanced performance. As shown in Table 20, pairwise classifiers show statistically significant performance when predicting recurrence within the subset of patients with positive lymph node involvement based on Wilcox P-value and a number of other metrics. Each classifier in Table 20 is described by the machine learning algorithm that combines the markers (column ‘classifier’) as well as the probeset ID number of the corresponding markers as provided by Affymetrix (http://www.affymetrix.com/analysis/index.affx; Table 16). These classifiers were trained and tuned in the discover cohort (n=133) and the performance on the positive lymph node involvement patients from the validation set is indicated. When dichotomization is necessary for a performance metric, the classifier scores are dichotomized using unsupervised clustering in the pamr package. The machine learning algorithms used are Naïve Bayes (NB), recursive Partitioning (Rpart), Support Vector Machines (SVMs), Random Forest (RF) and K Nearest Neighbors (KNN). These machine learning algorithms were executed with default parameters using packages rpart 4.1-0, HDclassif 1.2.2, randomForest 4.6-7, caret 5.15-61, cluster 1.14.3, e1071 1.6-1, class 7.3-5 in R. Each classifier generates a score between 0 and 1, except for SVM which generates a score from -09 to +co; in all cases, higher score values indicate higher probability of recurrence.

These results showed that the methods and markers of the present invention are useful for prognosing muscle invasive bladder cancer recurrence. These results further showed that the methods and markers of the present invention are useful for diagnosing, prognosing, determining the progression of cancer, or predicting benefit from therapy in a subject having cancer.

TABLE 16

| | AFFYMETRIX | | | | | | |
|---|---|---|---|---|---|---|---|
| | PROBESET | | | CHRO- | | | |
| SEQ ID NO | ID | GENES | CATEGORY | MOSOME | START | END | STRAND |
| SEQ ID NO. 16 | 2318654 | VAMP3 | Non-coding (UTR) | chr1 | 7841394 | 7841457 | + |
| SEQ ID NO. 17 | 2318755 | PARK7 | Non-coding (UTR) | chr1 | 8045210 | 8045305 | + |
| SEQ ID NO. 18 | 2320062 | TARDBP | Coding | chr1 | 11082181 | 11082249 | + |
| SEQ ID NO. 19 | 2321661 | TMEM51 | Non-coding (UTR) | chr1 | 15546921 | 15546958 | + |
| SEQ ID NO. 20 | 2322383 | C1orf144 | Non-coding (UTR) | chr1 | 16722804 | 16722848 | + |
| SEQ ID NO. 21 | 2325809 | TMEM50A | Non-coding (UTR) | chr1 | 25687219 | 25687391 | + |
| SEQ ID NO. 22 | 2327494 | SNHG3 | Non- | chr1 | 28836076 | 28836118 | + |
| | | | coding (ncTranscript) | | | | |
| SEQ ID NO. 23 | 2327865 | PTPRU | Non-coding (UTR) | chr1 | 29653253 | 29653286 | + |
| SEQ ID NO. 24 | 2328504 | KHDRBS1 | Non-coding (UTR) | chr1 | 32509334 | 32509457 | + |
| SEQ ID NO. 25 | 2328507 | KHDRBS1 | Non- | chr1 | 32510473 | 32510602 | + |
| | | | coding (Intronic) | | | | |
| SEQ ID NO. 26 | 2329019 | RBBP4 | Coding | chr1 | 33145365 | 33145408 | + |
| SEQ ID NO. 27 | 2329798 | ZMYM4 | Coding | chr1 | 35858255 | 35858285 | + |
| SEQ ID NO. 28 | 2330704 | ZC3H12A | Non-coding (UTR) | chr1 | 37949930 | 37949963 | + |
| SEQ ID NO. 29 | 2331419 | MACF1 | Coding | chr1 | 39888103 | 39888203 | + |
| SEQ ID NO. 30 | 2331842 | ZMPSTE24 | Non-coding (UTR) | chr1 | 40759698 | 40759778 | + |
| SEQ ID NO. 31 | 2332192 | CTPS | Non-coding (UTR) | chr1 | 41478182 | 41478210 | + |
| SEQ ID NO. 32 | 2332740 | PPIH | Non-coding (UTR) | chr1 | 43142355 | 43142406 | + |
| SEQ ID NO. 33 | 2334302 | UROD | Non-coding (UTR) | chr1 | 45481172 | 45481224 | + |
| SEQ ID NO. 34 | 2336960 | TMEM59 | Non-coding (Exonic | chr1 | 54497388 | 54497746 | + |
| | | | Antisense) | | | | |
| SEQ ID NO. 35 | 2339310 | INADL | Non-coding (UTR) | chr1 | 62628807 | 62628976 | + |
| SEQ ID NO. 36 | 2342617 | ACADM | Non-coding (UTR) | chr1 | 76229076 | 76229135 | + |
| SEQ ID NO. 37 | 2345634 | PKN2 | Coding | chr1 | 89206755 | 89206948 | + |
| SEQ ID NO. 38 | 2345663 | PKN2 | Non- | chr1 | 89262619 | 89262697 | + |
| | | | coding (Intronic) | | | | |
| SEQ ID NO. 39 | 2345666 | PKN2 | Coding | chr1 | 89270538 | 89270576 | + |
| SEQ ID NO. 40 | 2346911 | RPL5 | Coding | chr1 | 93307350 | 93307383 | + |
| SEQ ID NO. 41 | 2346912 | RPL5 | Non-coding (UTR) | chr1 | 93307433 | 93307475 | + |
| SEQ ID NO. 42 | 2347108 | DR1 | Non-coding (UTR) | chr1 | 93826315 | 93826475 | + |
| SEQ ID NO. 43 | 2348819 | CCDC76 | Non-coding (UTR) | chr1 | 100615958 | 100616029 | + |
| SEQ ID NO. 44 | 2350305 | PRPF38B | Coding | chr1 | 109242408 | 109242557 | + |
| SEQ ID NO. 45 | 2350371 | STXBP3 | Coding | chr1 | 109325090 | 109325138 | + |
| SEQ ID NO. 46 | 2352268 | CAPZA1 | Non-coding (UTR) | chr1 | 113212895 | 113214104 | + |
| SEQ ID NO. 47 | 2353492 | ATP1A1 | Coding | chr1 | 116926636 | 116926663 | + |
| SEQ ID NO. 48 | 2358275 | RPRD2 | Coding | chr1 | 150432735 | 150432779 | + |
| SEQ ID NO. 49 | 2358923 | PSMB4 | Non-coding (UTR) | chr1 | 151374317 | 151374387 | + |
| SEQ ID NO. 50 | 2359857 | INTS3 | Coding | chr1 | 153735226 | 153735307 | + |
| SEQ ID NO. 51 | 2360078 | C1orf43 | Non-coding (Exonic | chr1 | 154179232 | 154179785 | + |
| | | | Antisense) | | | | |
| SEQ ID NO. 52 | 2360119 | UBAP2L | Coding | chr1 | 154227635 | 154227752 | + |
| SEQ ID NO. 53 | 2360120 | UBAP2L | Coding | chr1 | 154227763 | 154227790 | + |
| SEQ ID NO. 54 | 2360340 | UBE2Q1 | Non-coding (Exonic | chr1 | 154522576 | 154522811 | + |
| | | | Antisense) | | | | |
| SEQ ID NO. 55 | 2360716 | SLC50A1 | Non-coding (UTR) | chr1 | 155111252 | 155111301 | + |
| SEQ ID NO. 56 | 2360962 | ASH1L | Non-coding (Exonic | chr1 | 155490977 | 155491324 | + |
| | | | Antisense) | | | | |
| SEQ ID NO. 57 | 2361031 | MSTO1 | Non-coding (UTR) | chr1 | 155584682 | 155584715 | + |
| SEQ ID NO. 58 | 2363064 | DCAF8 | Non-coding (Exonic | chr1 | 160186563 | 160186594 | + |
| | | | Antisense) | | | | |
| SEQ ID NO. 59 | 2363334 | F11R | Non-coding (Exonic | chr1 | 160966066 | 160966286 | + |
| | | | Antisense) | | | | |
| SEQ ID NO. 60 | 2363956 | ATF6 | Coding | chr1 | 161823014 | 161823046 | + |
| SEQ ID NO. 61 | 2365991 | MPZL1 | Coding | chr1 | 167757129 | 167757158 | + |
| SEQ ID NO. 62 | 2367178 | PRRC2C | Coding | chr1 | 171492580 | 171492650 | + |
| SEQ ID NO. 63 | 2367214 | PRRC2C | Coding | chr1 | 171548499 | 171548532 | + |
| SEQ ID NO. 64 | 2367236 | PRRC2C | Non- | chr1 | 171457106 | 171457157 | + |
| | | | coding (Intronic) | | | | |
| SEQ ID NO. 65 | 2369830 | chr1+: 179888850-179889162 | Non- | chr1 | 179888850 | 179889162 | + |
| | | | coding (Intergenic) | | | | |
| SEQ ID NO. 66 | 2370014 | QSOX1 | Non-coding (UTR) | chr1 | 180167171 | 180167222 | + |
| SEQ ID NO. 67 | 2371291 | SMG7 | Coding | chr1 | 183511357 | 183511473 | + |
| SEQ ID NO. 68 | 2371339 | ARPC5 | Non-coding (Exonic | chr1 | 183595532 | 183595591 | + |
| | | | Antisense) | | | | |
| SEQ ID NO. 69 | 2371796 | IVNS1ABP | Non-coding (Exonic | chr1 | 185280292 | 185280436 | + |
| | | | Antisense) | | | | |
| SEQ ID NO. 70 | 2373002 | CDC73 | Coding | chr1 | 193181573 | 193181606 | + |
| SEQ ID NO. 71 | 2375035 | ELF3 | Non-coding (UTR) | chr1 | 201985159 | 201985198 | + |
| SEQ ID NO. 72 | 2375037 | ELF3 | Non-coding (UTR) | chr1 | 201986251 | 201986287 | + |
| SEQ ID NO. 73 | 2375675 | BTG2 | Non-coding (UTR) | chr1 | 203278631 | 203278704 | + |
| SEQ ID NO. 74 | 2375780 | ATP2B4 | Non-coding (UTR) | chr1 | 203713164 | 203713195 | + |
| SEQ ID NO. 75 | 2375850 | ZC3H11A | Coding | chr1 | 203816372 | 203816525 | + |
| SEQ ID NO. 76 | 2375890 | SNRPE | Non-coding (UTR) | chr1 | 203839160 | 203839185 | + |
| SEQ ID NO. 77 | 2377473 | CD46 | Non- | chr1 | 207940689 | 207940924 | + |
| | | | coding (ncTranscript) | | | | |
| SEQ ID NO. 78 | 2377508 | CD46 | Non-coding (UTR) | chr1 | 207968669 | 207968799 | + |
| SEQ ID NO. 79 | 2378615 | RCOR3 | Coding | chr1 | 211469018 | 211469048 | + |
| SEQ ID NO. 80 | 2379787 | SMYD2 | Coding | chr1 | 214498021 | 214498050 | + |
| SEQ ID NO. 81 | 2383763 | ARF1 | Non-coding (UTR) | chr1 | 228285909 | 228286156 | + |
| SEQ ID NO. 82 | 2389076 | FAM36A | Non- | chr1 | 245005918 | 245006248 | + |
| | | | coding (Intronic) | | | | |
| SEQ ID NO. 83 | 2389079 | FAM36A | Non-coding (UTR) | chr1 | 245006641 | 245006814 | + |
| SEQ ID NO. 84 | 2389084 | NCRNA00201 | Non-coding (Intronic | chr1 | 245010244 | 245010313 | + |
| | | | Antisense) | | | | |
| SEQ ID NO. 85 | 2389086 | NCRNA00201 | Non-coding (Intronic | chr1 | 245010957 | 245011086 | + |
| | | | Antisense) | | | | |
| SEQ ID NO. 86 | 2389817 | SCCPDH | Non-coding (UTR) | chr1 | 246930619 | 246930723 | + |
| SEQ ID NO. 87 | 2394560 | RPL22 | Non-coding (UTR) | chr1 | 6245095 | 6245147 | − |
| SEQ ID NO. 88 | 2395192 | ERRF11 | Non- | chr1 | 8077370 | 8078230 | − |
| | | | coding (Intronic) | | | | |
| SEQ ID NO. 89 | 2395195 | ERRF11 | Non- | chr1 | 8081604 | 8082267 | − |
| | | | coding (Intronic) | | | | |
| SEQ ID NO. 90 | 2396421 | MASP2 | Coding | chr1 | 11086948 | 11086983 | − |
| SEQ ID NO. 91 | 2400180 | CAMK2N1 | Non-coding (UTR) | chr1 | 20810077 | 20810117 | − |
| SEQ ID NO. 92 | 2400323 | HP1BP3 | Non-coding (UTR) | chr1 | 21069239 | 21069434 | − |
| SEQ ID NO. 93 | 2401277 | chr1−: 23614583-23614893 | Non- | chr1 | 23614583 | 23614893 | − |
| | | | coding (Intergenic) | | | | |
| SEQ ID NO. 94 | 2401288 | HNRNPR | Non-coding (UTR) | chr1 | 23636701 | 23636918 | − |
| SEQ ID NO. 95 | 2401350 | TCEA3 | Non-coding (UTR) | chr1 | 23707883 | 23707923 | − |
| SEQ ID NO. 96 | 2401368 | TCEA3 | Coding | chr1 | 23735157 | 23735185 | − |
| SEQ ID NO. 97 | 2402210 | TMEM50A | Non-coding (Exonic | chr1 | 25687170 | 25687537 | − |
| | | | Antisense) | | | | |
| SEQ ID NO. 98 | 2405379 | AK2 | Coding | chr1 | 33478681 | 33478714 | − |
| SEQ ID NO. 99 | 2406094 | SFPQ | Coding | chr1 | 35654644 | 35654687 | − |
| SEQ ID NO. 100 | 2406148 | KIAA0319L | Non-coding (UTR) | chr1 | 35899093 | 35899130 | − |
| SEQ ID NO. 101 | 2407134 | MEAF6 | Non-coding (UTR) | chr1 | 37958790 | 37958867 | − |
| SEQ ID NO. 102 | 2408880 | FOXJ3 | Coding | chr1 | 42660561 | 42660688 | − |
| SEQ ID NO. 103 | 2410112 | MUTYH | Non-coding (UTR) | chr1 | 45794927 | 45794963 | − |
| SEQ ID NO. 104 | 2410528 | POMGNT1 | Coding | chr1 | 46654372 | 46654408 | − |
| SEQ ID NO. 105 | 2412669 | TXNDC12 | Non-coding (UTR) | chr1 | 52485854 | 52485894 | − |
| SEQ ID NO. 106 | 2413580 | TMEM59 | Non-coding (UTR) | chr1 | 54497716 | 54497788 | − |
| SEQ ID NO. 107 | 2414030 | USP24 | Coding | chr1 | 55608416 | 55608446 | − |
| SEQ ID NO. 108 | 2414960 | TACSTD2 | Non-coding (UTR) | chr1 | 59041241 | 59041545 | − |
| SEQ ID NO. 109 | 2417788 | SRSF11 | Non-coding (Exonic | chr1 | 70717148 | 70717519 | − |
| | | | Antisense) | | | | |
| SEQ ID NO. 110 | 2418028 | ZRANB2 | Coding | chr1 | 71542488 | 71542559 | − |
| SEQ ID NO. 111 | 2419242 | FUBP1 | Non- | chr1 | 78412683 | 78413071 | − |
| | | | coding (ncTranscript) | | | | |
| SEQ ID NO. 112 | 2419247 | FUBP1 | Non- | chr1 | 78413793 | 78413946 | − |
| | | | coding (ncTranscript) | | | | |
| SEQ ID NO. 113 | 2419276 | FUBP1 | Coding | chr1 | 78429939 | 78430038 | − |
| SEQ ID NO. 114 | 2420740 | SYDE2 | Non-coding (UTR) | chr1 | 85624387 | 85624416 | − |
| SEQ ID NO. 115 | 2420839 | DDAH1 | Non-coding (UTR) | chr1 | 85784191 | 85784259 | − |
| SEQ ID NO. 116 | 2421273 | RP4-604K5.1 | Non-coding (UTR) | chr1 | 87328509 | 87329064 | − |
| SEQ ID NO. 117 | 2421274 | RP4-604K5.1 | Coding | chr1 | 87329188 | 87329229 | − |
| SEQ ID NO. 118 | 2421932 | GBP2 | Non-coding (UTR) | chr1 | 89573754 | 89573847 | − |
| SEQ ID NO. 119 | 2422519 | ZNF644 | Non-coding (UTR) | chr1 | 91381226 | 91381289 | − |
| SEQ ID NO. 120 | 2423200 | RPL5 | Non-coding (Exonic | chr1 | 93303053 | 93303092 | − |
| | | | Antisense) | | | | |
| SEQ ID NO. 121 | 2427009 | SORT1 | Non-coding (UTR) | chr1 | 109852227 | 109852265 | − |
| SEQ ID NO. 122 | 2427501 | HBXIP | Non-coding (UTR) | chr1 | 110943961 | 110944044 | − |
| SEQ ID NO. 123 | 2427978 | ATP5F1 | Non-coding (Exonic | chr1 | 112003809 | 112003849 | − |
| | | | Antisense) | | | | |
| SEQ ID NO. 124 | 2428394 | CAPZA1 | Non-coding (Exonic | chr1 | 113213521 | 113214105 | − |
| | | | Antisense) | | | | |
| SEQ ID NO. 125 | 2429071 | TRIM33 | Non-coding (UTR) | chr1 | 114938592 | 114939061 | − |
| SEQ ID NO. 126 | 2429119 | TRIM33 | Non- | chr1 | 114969301 | 114969662 | − |
| | | | coding (Intronic) | | | | |
| SEQ ID NO. 127 | 2429168 | BCAS2 | Coding | chr1 | 115110821 | 115110850 | − |
| SEQ ID NO. 128 | 2429282 | CSDE1 | Non-coding (UTR) | chr1 | 115260444 | 115260566 | − |
| SEQ ID NO. 129 | 2429309 | CSDE1 | Coding | chr1 | 115275269 | 115275381 | − |
| SEQ ID NO. 130 | 2429310 | CSDE1 | Coding | chr1 | 115276377 | 115276466 | − |
| SEQ ID NO. 131 | 2433376 | ACP6 | Non-coding (UTR) | chr1 | 147119193 | 147119224 | − |
| SEQ ID NO. 132 | 2434721 | LASS2 | Non-coding (UTR) | chr1 | 150937780 | 150937854 | − |
| SEQ ID NO. 133 | 2435391 | S100A10 | Non-coding (UTR) | chr1 | 151955516 | 151955638 | − |
| SEQ ID NO. 134 | 2437095 | KRTCAP2 | Non-coding (UTR) | chr1 | 155141888 | 155141915 | − |
| SEQ ID NO. 135 | 2437537 | RP11-29H23.5 | Non- | chr1 | 155584631 | 155584658 | − |
| | | | coding (ncTranscript) | | | | |
| SEQ ID NO. 136 | 2437538 | RP11-29H23.5 | Non- | chr1 | 155584675 | 155584710 | − |
| | | | coding (Intronic) | | | | |
| SEQ ID NO. 137 | 2437680 | GON4L | Coding | chr1 | 155740950 | 155740999 | − |
| SEQ ID NO. 138 | 2437775 | KIAA0907 | Coding | chr1 | 155891652 | 155891707 | − |
| SEQ ID NO. 139 | 2439041 | RP11-367J7.4 | Non-coding (Exonic | chr1 | 157678937 | 157679031 | − |
| | | | Antisense) | | | | |
| SEQ ID NO. 140 | 2440149 | COPA | Coding | chr1 | 160259963 | 160259992 | − |
| SEQ ID NO. 141 | 2440513 | TSTD1 | Non-coding (UTR) | chr1 | 161007434 | 161007466 | − |
| SEQ ID NO. 142 | 2442702 | CREG1 | Non-coding (UTR) | chr1 | 167510554 | 167511025 | − |
| SEQ ID NO. 143 | 2443917 | PRRC2C | Non-coding (Exonic | chr1 | 171492584 | 171492848 | − |
| | | | Antisense) | | | | |
| SEQ ID NO. 144 | 2444488 | GAS5 | Non- | chr1 | 173833813 | 173833842 | − |
| | | | coding (ncTranscript) | | | | |
| SEQ ID NO. 145 | 2444489 | GAS5, SNORD80 | Non- | chr1 | 173833844 | 173833968 | − |
| | | | coding (ncTranscript) | | | | |
| SEQ ID NO. 146 | 2448289 | TPR | Coding | chr1 | 186316431 | 186316560 | − |
| SEQ ID NO. 147 | 2448313 | TPR | Coding | chr1 | 186340102 | 186340137 | − |
| SEQ ID NO. 148 | 2450669 | TMEM9 | Non-coding (UTR) | chr1 | 201103905 | 201103934 | − |
| SEQ ID NO. 149 | 2451314 | KDM5B | Non-coding (UTR) | chr1 | 202697851 | 202698124 | − |
| SEQ ID NO. 150 | 2452652 | NUCKS1 | Coding | chr1 | 205693063 | 205693090 | − |
| SEQ ID NO. 151 | 2452655 | NUCKS1 | Coding | chr1 | 205698706 | 205698745 | − |
| SEQ ID NO. 152 | 2457626 | BROX | Non-coding (Exonic | chr1 | 222906823 | 222906871 | − |
| | | | Antisense) | | | | |
| SEQ ID NO. 153 | 2458075 | PARP1 | Non- | chr1 | 224563524 | 224563621 | − |
| | | | coding (Intronic) | | | | |
| SEQ ID NO. 154 | 2458381 | ENAH | Coding | chr1 | 225700375 | 225700414 | − |
| SEQ ID NO. 155 | 2458503 | PARP1 | Non- | chr1 | 225977149 | 225977173 | − |
| | | | coding (Intronic) | | | | |
| SEQ ID NO. 156 | 2459655 | RHOU | Non-coding (Exonic | chr1 | 228882329 | 228882370 | − |
| | | | Antisense) | | | | |
| SEQ ID NO. 157 | 2461827 | ARID4B | Coding | chr1 | 235383150 | 235383265 | − |
| SEQ ID NO. 158 | 2461828 | ARID4B | Coding | chr1 | 235383674 | 235383841 | − |
| SEQ ID NO. 159 | 2462178 | NID1 | Non-coding (UTR) | chr1 | 236139156 | 236139261 | − |
| SEQ ID NO. 160 | 2464496 | NCRNA00201 | Non- | chr1 | 245010247 | 245010645 | − |
| | | | coding (Intronic) | | | | |
| SEQ ID NO. 161 | 2464505 | HNRNPU | Non-coding (UTR) | chr1 | 245016420 | 245016449 | − |
| SEQ ID NO. 162 | 2464514 | HNRNPU, NCRNA00201 | Coding | chr1 | 245017752 | 245017789 | − |
| SEQ ID NO. 163 | 2464537 | HNRNPU | Coding | chr1 | 245022587 | 245022672 | − |
| SEQ ID NO. 164 | 2468935 | CPSF3 | Coding | chr2 | 9580633 | 9580745 | + |
| SEQ ID NO. 165 | 2469568 | ATP6V1C2 | Non-coding (UTR) | chr2 | 10923651 | 10923817 | + |
| SEQ ID NO. 166 | 2473241 | NCOA1 | Coding | chr2 | 24962301 | 24962395 | + |
| SEQ ID NO. 167 | 2473624 | RAB10 | Non-coding (UTR) | chr2 | 26358253 | 26358315 | + |
| SEQ ID NO. 168 | 2474147 | TMEM214 | Non-coding (UTR) | chr2 | 27264519 | 27264553 | + |
| SEQ ID NO. 169 | 2474700 | GPN1 | Coding | chr2 | 27873018 | 27873055 | + |
| SEQ ID NO. 170 | 2474770 | MRPL33 | Non-coding (UTR) | chr2 | 28002513 | 28002557 | + |
| SEQ ID NO. 171 | 2475647 | YPEL5 | Non-coding (UTR) | chr2 | 30383149 | 30383365 | + |
| SEQ ID NO. 172 | 2476316 | BIRC6 | Coding | chr2 | 32707551 | 32707640 | + |
| SEQ ID NO. 173 | 2476408 | BIRC6 | Non-coding (UTR) | chr2 | 32843548 | 32843578 | + |
| SEQ ID NO. 174 | 2477341 | AC007390.5 | Coding | chr2 | 37428785 | 37428822 | + |
| SEQ ID NO. 175 | 2478839 | EML4 | Non-coding (UTR) | chr2 | 42559443 | 42559634 | + |
| SEQ ID NO. 176 | 2479729 | SLC3A1 | Non- | chr2 | 44546007 | 44546621 | + |
| | | | coding (Intronic) | | | | |
| SEQ ID NO. 177 | 2480989 | EPCAM | Non-coding (UTR) | chr2 | 47614066 | 47614142 | + |
| SEQ ID NO. 178 | 2481178 | MSH6 | Non-coding (UTR) | chr2 | 48034001 | 48034074 | + |
| SEQ ID NO. 179 | 2482593 | SPTBN1 | Coding | chr2 | 54882207 | 54882334 | + |
| SEQ ID NO. 180 | 2482624 | SPTBN1 | Non-coding (UTR) | chr2 | 54896727 | 54896801 | + |
| SEQ ID NO. 181 | 2483047 | CCDC104 | Non-coding (UTR) | chr2 | 55772160 | 55772187 | + |
| SEQ ID NO. 182 | 2484558 | AHSA2 | Non-coding (UTR) | chr2 | 61405713 | 61405771 | + |
| SEQ ID NO. 183 | 2484807 | COMMD1 | Non-coding (UTR) | chr2 | 62363157 | 62363187 | + |
| SEQ ID NO. 184 | 2485214 | MDH1 | Non-coding (UTR) | chr2 | 63834172 | 63834310 | + |
| SEQ ID NO. 185 | 2485276 | UGP2 | Coding | chr2 | 64111133 | 64111216 | + |
| SEQ ID NO. 186 | 2487216 | ANTXR1 | Non-coding (UTR) | chr2 | 69475036 | 69475067 | + |
| SEQ ID NO. 187 | 2487645 | PCBP1 | Non-coding (UTR) | chr2 | 70315979 | 70316219 | + |
| SEQ ID NO. 188 | 2488150 | ZNF638 | Coding | chr2 | 71591148 | 71591240 | + |
| SEQ ID NO. 189 | 2489030 | ACTG2 | Non-coding (UTR) | chr2 | 74146720 | 74146765 | + |
| SEQ ID NO. 190 | 2489435 | HTRA2 | Non-coding (UTR) | chr2 | 74760238 | 74760277 | + |
| SEQ ID NO. 191 | 2491645 | MAT2A | Non-coding (UTR) | chr2 | 85771254 | 85771283 | + |
| SEQ ID NO. 192 | 2492101 | KDM3A | Coding | chr2 | 86705731 | 86705762 | + |
| SEQ ID NO. 193 | 2492833 | THNSL2 | Non-coding (UTR) | chr2 | 88486099 | 88486137 | + |
| SEQ ID NO. 194 | 2495140 | ACTR1B | Non-coding (Exonic | chr2 | 98272473 | 98272513 | + |
| | | | Antisense) | | | | |
| SEQ ID NO. 195 | 2498988 | GCC2 | Coding | chr2 | 109067518 | 109067567 | + |
| SEQ ID NO. 196 | 2507241 | CCNT2 | Non- | chr2 | 135704085 | 135704123 | + |
| | | | coding (Intronic) | | | | |
| SEQ ID NO. 197 | 2507514 | UBXN4 | Coding | chr2 | 136519410 | 136519481 | + |
| SEQ ID NO. 198 | 2507533 | UBXN4 | Non-coding (UTR) | chr2 | 136540632 | 136540870 | + |
| SEQ ID NO. 199 | 2512351 | MARCH7 | Coding | chr2 | 160604317 | 160604346 | + |
| SEQ ID NO. 200 | 2512363 | MARCH7 | Non- | chr2 | 160621087 | 160621136 | + |
| | | | coding (Intronic) | | | | |
| SEQ ID NO. 201 | 2512959 | GCA | Non-coding (UTR) | chr2 | 163217462 | 163217604 | + |
| SEQ ID NO. 202 | 2515085 | GORASP2 | Coding | chr2 | 171818173 | 171818259 | + |
| SEQ ID NO. 203 | 2517330 | HNRNPA3 | Non-coding (UTR) | chr2 | 178085947 | 178085971 | + |
| SEQ ID NO. 204 | 2518193 | UBE2E3 | Non- | chr2 | 181920652 | 181920789 | + |
| | | | coding (Intronic) | | | | |
| SEQ ID NO. 205 | 2518707 | NCKAP1 | Non-coding (Exonic | chr2 | 183789642 | 183790486 | + |
| | | | Antisense) | | | | |
| SEQ ID NO. 206 | 2525095 | CREB1 | Non-coding (UTR) | chr2 | 208462572 | 208462631 | + |
| SEQ ID NO. 207 | 2527206 | RPL37A | Coding | chr2 | 217366083 | 217366127 | + |
| SEQ ID NO. 208 | 2527635 | ARPC2 | Non- | chr2 | 219108586 | 219108713 | + |
| | | | coding (Intronic) | | | | |
| SEQ ID NO. 209 | 2532844 | ATG16L1 | Non-coding (UTR) | chr2 | 234204250 | 234204278 | + |
| SEQ ID NO. 210 | 2534447 | LRRFIP1 | Coding | chr2 | 238671957 | 238672019 | + |
| SEQ ID NO. 211 | 2537112 | SH3YL1 | Non-coding (UTR) | chr2 | 218176 | 218541 | − |
| SEQ ID NO. 212 | 2539775 | ITGB1BP1 | Non-coding (UTR) | chr2 | 9546826 | 9546962 | − |
| SEQ ID NO. 213 | 2540221 | NOL10 | Non-coding (UTR) | chr2 | 10711789 | 10711820 | − |
| SEQ ID NO. 214 | 2544180 | AC008073.5 | Non-coding (UTR) | chr2 | 24290483 | 24290599 | − |
| SEQ ID NO. 215 | 2544185 | AC008073.5 | Coding | chr2 | 24296946 | 24296980 | − |
| SEQ ID NO. 216 | 2544295 | ITSN2 | Coding | chr2 | 24498610 | 24498679 | − |
| SEQ ID NO. 217 | 2544306 | ITSN2 | Coding | chr2 | 24516558 | 24516637 | − |
| SEQ ID NO. 218 | 2546279 | SPDYA, PPP1CB | Non-coding (Exonic | chr2 | 29025630 | 29025776 | − |
| | | | Antisense) | | | | |
| SEQ ID NO. 219 | 2549323 | MAP4K3 | Coding | chr2 | 39552666 | 39552755 | − |
| SEQ ID NO. 220 | 2550840 | LRPPRC | Coding | chr2 | 44170831 | 44170960 | − |
| SEQ ID NO. 221 | 2550842 | LRPPRC | Coding | chr2 | 44173258 | 44173355 | − |
| SEQ ID NO. 222 | 2550964 | PREPL | Non-coding (UTR) | chr2 | 44546148 | 44546608 | − |
| SEQ ID NO. 223 | 2552021 | EPCAM | Non-coding (Exonic | chr2 | 47613985 | 47614122 | − |
| | | | Antisense) | | | | |
| SEQ ID NO. 224 | 2553312 | PSME4 | Coding | chr2 | 54127114 | 54127147 | − |
| SEQ ID NO. 225 | 2553583 | RTN4 | Non-coding (UTR) | chr2 | 55199333 | 55199382 | − |
| SEQ ID NO. 226 | 2553585 | RTN4 | Non-coding (UTR) | chr2 | 55199483 | 55199611 | − |
| SEQ ID NO. 227 | 2553595 | RTN4 | Coding | chr2 | 55200957 | 55200996 | − |
| SEQ ID NO. 228 | 2553907 | CCDC104 | Non-coding (Exonic | chr2 | 55771450 | 55771480 | − |
| | | | Antisense) | | | | |
| SEQ ID NO. 229 | 2554001 | PNPT1 | Coding | chr2 | 55899130 | 55899157 | − |
| SEQ ID NO. 230 | 2555279 | USP34 | Non-coding (UTR) | chr2 | 61414634 | 61414669 | − |
| SEQ ID NO. 231 | 2555281 | USP34 | Coding | chr2 | 61415253 | 61415312 | − |
| SEQ ID NO. 232 | 2555288 | USP34 | Coding | chr2 | 61417424 | 61417531 | − |
| SEQ ID NO. 233 | 2555330 | USP34 | Non- | chr2 | 61452805 | 61453037 | − |
| | | | coding (Intronic) | | | | |
| SEQ ID NO. 234 | 2555409 | USP34 | Coding | chr2 | 61575369 | 61575420 | − |
| SEQ ID NO. 235 | 2555411 | USP34 | Coding | chr2 | 61575891 | 61575998 | − |
| SEQ ID NO. 236 | 2555416 | USP34 | Coding | chr2 | 61597633 | 61597690 | − |
| SEQ ID NO. 237 | 2555507 | XPO1 | Coding | chr2 | 61712955 | 61713067 | − |
| SEQ ID NO. 238 | 2555522 | XPO1 | Coding | chr2 | 61725848 | 61725908 | − |
| SEQ ID NO. 239 | 2556229 | VPS54 | Coding | chr2 | 64126624 | 64126660 | − |
| SEQ ID NO. 240 | 2558208 | AAK1 | Coding | chr2 | 69754363 | 69754404 | − |
| SEQ ID NO. 241 | 2558313 | ANXA4 | Non-coding (Exonic | chr2 | 70053082 | 70053422 | − |
| | | | Antisense) | | | | |
| SEQ ID NO. 242 | 2558519 | TIA1 | Non-coding (UTR) | chr2 | 70439751 | 70439835 | − |
| SEQ ID NO. 243 | 2559293 | EXOC6B | Coding | chr2 | 72707745 | 72707845 | − |
| SEQ ID NO. 244 | 2562334 | MAT2A | Non-coding (Exonic | chr2 | 85771249 | 85771273 | − |
| | | | Antisense) | | | | |
| SEQ ID NO. 245 | 2565264 | SNRNP200 | Non-coding (UTR) | chr2 | 96940504 | 96940591 | − |
| SEQ ID NO. 246 | 2575142 | POLR2D | Non-coding (UTR) | chr2 | 128604260 | 128604288 | − |
| SEQ ID NO. 247 | 2577959 | DARS | Non-coding (UTR) | chr2 | 136664325 | 136664784 | − |
| SEQ ID NO. 248 | 2581588 | PRPF40A | Coding | chr2 | 153535585 | 153535623 | − |
| SEQ ID NO. 249 | 2583054 | BAZ2B | Coding | chr2 | 160255340 | 160255388 | − |
| SEQ ID NO. 250 | 2583056 | BAZ2B | Coding | chr2 | 160257114 | 160257145 | − |
| SEQ ID NO. 251 | 2583249 | MARCH7 | Non-coding (Exonic | chr2 | 160624839 | 160625024 | − |
| | | | Antisense) | | | | |
| SEQ ID NO. 252 | 2583609 | RBMS1 | Non-coding (UTR) | chr2 | 161129828 | 161129873 | − |
| SEQ ID NO. 253 | 2584845 | COBLL1 | Coding | chr2 | 165578935 | 165578973 | − |
| SEQ ID NO. 254 | 2588838 | NFE2L2 | Coding | chr2 | 178096686 | 178096714 | − |
| SEQ ID NO. 255 | 2590750 | NCKAP1 | Non-coding (UTR) | chr2 | 183789825 | 183790389 | − |
| SEQ ID NO. 256 | 2590802 | NCKAP1 | Coding | chr2 | 183867709 | 183867744 | − |
| SEQ ID NO. 257 | 2591635 | COL3A1 | Non-coding (Exonic | chr2 | 189873799 | 189873913 | − |
| | | | Antisense) | | | | |
| SEQ ID NO. 258 | 2591638 | COL3A1 | Non-coding (Exonic | chr2 | 189876571 | 189877301 | − |
| | | | Antisense) | | | | |
| SEQ ID NO. 259 | 2593447 | PGAP1 | Coding | chr2 | 197767344 | 197767458 | − |
| SEQ ID NO. 260 | 2593692 | SF3B1 | Coding | chr2 | 198265439 | 198265473 | − |
| SEQ ID NO. 261 | 2593714 | SF3B1 | Coding | chr2 | 198283234 | 198283287 | − |
| SEQ ID NO. 262 | 2593758 | HSPD1 | Coding | chr2 | 198359404 | 198359433 | − |
| SEQ ID NO. 263 | 2594506 | CLK1 | Coding | chr2 | 201719352 | 201719417 | − |
| SEQ ID NO. 264 | 2596691 | FAM119A | Non-coding (UTR) | chr2 | 208446110 | 208446140 | − |
| SEQ ID NO. 265 | 2597013 | IDH1 | Non-coding (UTR) | chr2 | 209101126 | 209101382 | − |
| SEQ ID NO. 266 | 2598267 | FN1 | Non-coding (UTR) | chr2 | 216226027 | 216226061 | − |
| SEQ ID NO. 267 | 2598289 | FN1 | Coding | chr2 | 216237027 | 216237060 | − |
| SEQ ID NO. 268 | 2598781 | RPL37A, AC073321.5 | Non-coding (Exonic | chr2 | 217366067 | 217366101 | − |
| | | | Antisense) | | | | |
| SEQ ID NO. 269 | 2599342 | ARPC2 | Non-coding (Exonic | chr2 | 219118929 | 219119040 | − |
| | | | Antisense) | | | | |
| SEQ ID NO. 270 | 2599903 | C2orf24 | Non-coding (UTR) | chr2 | 220036632 | 220036691 | − |
| SEQ ID NO. 271 | 2606647 | MYEOV2 | Non-coding (UTR) | chr2 | 241070273 | 241070379 | − |
| SEQ ID NO. 272 | 2607152 | HDLBP | Coding | chr2 | 242186485 | 242186548 | − |
| SEQ ID NO. 273 | 2607374 | DTYMK | Non-coding (UTR) | chr2 | 242615202 | 242615297 | − |
| SEQ ID NO. 274 | 2609635 | SETD5 | Non- | chr3 | 9473392 | 9473435 | + |
| | | | coding (ncTranscript) | | | | |
| SEQ ID NO. 275 | 2609657 | SETD5 | Coding | chr3 | 9482211 | 9482361 | + |
| SEQ ID NO. 276 | 2609668 | SETD5 | Coding | chr3 | 9486849 | 9486982 | + |
| SEQ ID NO. 277 | 2610353 | chr3+: 10195215-10195245 | Non- | chr3 | 10195215 | 10195245 | + |
| | | | coding (Intergenic) | | | | |
| SEQ ID NO. 278 | 2611083 | PPARG | Non- | chr3 | 12400965 | 12401103 | + |
| | | | coding (Intronic) | | | | |
| SEQ ID NO. 279 | 2614100 | UBE2E1 | Coding | chr3 | 23929058 | 23929154 | + |
| SEQ ID NO. 280 | 2614135 | RPL15 | Non-coding (UTR) | chr3 | 23961672 | 23961706 | + |
| SEQ ID NO. 281 | 2614137 | RPL15 | Non-coding (UTR) | chr3 | 23962179 | 23962307 | + |
| SEQ ID NO. 282 | 2615656 | STT3B | Non-coding (UTR) | chr3 | 31678984 | 31679087 | + |
| SEQ ID NO. 283 | 2616071 | CNOT10 | Non-coding (UTR) | chr3 | 32815151 | 32815180 | + |
| SEQ ID NO. 284 | 2616248 | FBXL2 | Non- | chr3 | 33429931 | 33430929 | + |
| | | | coding (Intronic) | | | | |
| SEQ ID NO. 285 | 2617578 | MYD88 | Non-coding (UTR) | chr3 | 38184424 | 38184480 | + |
| SEQ ID NO. 286 | 2618656 | RPL14 | Non-coding (UTR) | chr3 | 40503816 | 40503845 | + |
| SEQ ID NO. 287 | 2619365 | NKTR | Non- | chr3 | 42666725 | 42666819 | + |
| | | | coding (Intronic) | | | | |
| SEQ ID NO. 288 | 2625632 | APPL1 | Coding | chr3 | 57290985 | 57291016 | + |
| SEQ ID NO. 289 | 2625643 | APPL1 | Coding | chr3 | 57301770 | 57301820 | + |
| SEQ ID NO. 290 | 2625893 | SLMAP | Non-coding (UTR) | chr3 | 57914855 | 57914884 | + |
| SEQ ID NO. 291 | 2627951 | RP11-714G12.1 | Non- | chr3 | 65673641 | 65673667 | + |
| | | | coding (ncTranscript) | | | | |
| SEQ ID NO. 292 | 2633534 | C3orf26 | Non-coding (UTR) | chr3 | 99897391 | 99897425 | + |
| SEQ ID NO. 293 | 2633651 | NIT2 | Non-coding (UTR) | chr3 | 100074128 | 100074184 | + |
| SEQ ID NO. 294 | 2638377 | LRRC58 | Non-coding (Exonic | chr3 | 120048985 | 120049161 | + |
| | | | Antisense) | | | | |
| SEQ ID NO. 295 | 2638451 | NDUFB4 | Coding | chr3 | 120321084 | 120321117 | + |
| SEQ ID NO. 296 | 2638452 | NDUFB4 | Non-coding (UTR) | chr3 | 120321127 | 120321167 | + |
| SEQ ID NO. 297 | 2641575 | COPG | Non-coding (UTR) | chr3 | 128996540 | 128996609 | + |
| SEQ ID NO. 298 | 2643579 | CEP63 | Non-coding (UTR) | chr3 | 134280433 | 134280587 | + |
| SEQ ID NO. 299 | 2645294 | SLC25A36 | Non-coding (UTR) | chr3 | 140685221 | 140685276 | + |
| SEQ ID NO. 300 | 2646066 | U2SURP | Coding | chr3 | 142741771 | 142741883 | + |
| SEQ ID NO. 301 | 2646085 | U2SURP | Coding | chr3 | 142757736 | 142757777 | + |
| SEQ ID NO. 302 | 2647361 | WWTR1 | Non-coding (Exonic | chr3 | 149235100 | 149235425 | + |
| | | | Antisense) | | | | |
| SEQ ID NO. 303 | 2647678 | TSC22D2 | Non- | chr3 | 150143909 | 150143956 | + |
| | | | coding (Intronic) | | | | |
| SEQ ID NO. 304 | 2647775 | EIF2A | Coding | chr3 | 150293465 | 150293498 | + |
| SEQ ID NO. 305 | 2647779 | EIF2A | Coding | chr3 | 15030098 | 150301017 | + |
| SEQ ID NO. 306 | 2648202 | MBNL1 | Coding | chr3 | 152177060 | 152177091 | + |
| SEQ ID NO. 307 | 2651521 | MECOM | Non-coding (Intronic | chr3 | 169048129 | 169048401 | + |
| | | | Antisense) | | | | |
| SEQ ID NO. 308 | 2652021 | chr3+: 170114495-170114579 | Non- | chr3 | 170114495 | 170114579 | + |
| | | | coding (Intergenic) | | | | |
| SEQ ID NO. 309 | 2654335 | TTC14 | Coding | chr3 | 180328005 | 180328126 | + |
| SEQ ID NO. 310 | 2654874 | ATP11B | Coding | chr3 | 182605374 | 182605485 | + |
| SEQ ID NO. 311 | 2654878 | ATP11B | Coding | chr3 | 182614525 | 182614552 | + |
| SEQ ID NO. 312 | 2655475 | DVL3 | Non-coding (UTR) | chr3 | 183891141 | 183891343 | + |
| SEQ ID NO. 313 | 2655509 | AP2M1 | Non-coding (UTR) | chr3 | 183901799 | 183901823 | + |
| SEQ ID NO. 314 | 2655678 | PSMD2 | Coding | chr3 | 184024162 | 184024203 | + |
| SEQ ID NO. 315 | 2656791 | EIF4A2 | Non-coding (UTR) | chr3 | 186507573 | 186507657 | + |
| SEQ ID NO. 316 | 2658354 | OPA1 | Coding | chr3 | 193332585 | 193332691 | + |
| SEQ ID NO. 317 | 2658632 | HES1 | Non-coding (UTR) | chr3 | 193856166 | 193856286 | + |
| SEQ ID NO. 318 | 2663553 | NUP210 | Non-coding (UTR) | chr3 | 13357767 | 13357827 | − |
| SEQ ID NO. 319 | 2666503 | TOP2B | Coding | chr3 | 25659949 | 25659979 | − |
| SEQ ID NO. 320 | 2666519 | TOP2B | Coding | chr3 | 25668695 | 25668787 | − |
| SEQ ID NO. 321 | 2666524 | TOP2B | Coding | chr3 | 25671534 | 25671588 | − |
| SEQ ID NO. 322 | 2669316 | GOLGA4 | Non-coding (Exonic | chr3 | 37365990 | 37366061 | − |
| | | | Antisense) | | | | |
| SEQ ID NO. 323 | 2669571 | MYD88 | Non-coding (Exonic | chr3 | 38184382 | 38184448 | − |
| | | | Antisense) | | | | |
| SEQ ID NO. 324 | 2670438 | chr3−: 41197321-41197352 | Non- | chr3 | 41197321 | 41197352 | − |
| | | | coding (Intergenic) | | | | |
| SEQ ID NO. 325 | 2672775 | C3orf75 | Non-coding (UTR) | chr3 | 47537138 | 47537165 | − |
| SEQ ID NO. 326 | 2674243 | RHOA | Non-coding (UTR) | chr3 | 49396583 | 49396613 | − |
| SEQ ID NO. 327 | 2675269 | NPRL2 | Non-coding (UTR) | chr3 | 50384946 | 50384986 | − |
| SEQ ID NO. 328 | 2675802 | PCBP4 | Non-coding (UTR) | chr3 | 51991509 | 51991574 | − |
| SEQ ID NO. 329 | 2676931 | RP11-884K10.5 | Non-coding (UTR) | chr3 | 53919264 | 53919310 | − |
| SEQ ID NO. 330 | 2677655 | C3orf63 | Non-coding (UTR) | chr3 | 56656912 | 56656956 | − |
| SEQ ID NO. 331 | 2677695 | C3orf63 | Non- | chr3 | 56702382 | 56702424 | − |
| | | | coding (Intronic) | | | | |
| SEQ ID NO. 332 | 2680511 | MAGI1, RP11- | Non- | chr3 | 65887126 | 65887154 | − |
| | | 175F9.3 | coding (Intronic) | | | | |
| SEQ ID NO. 333 | 2682663 | PPP4R2 | Non-coding (Exonic | chr3 | 73116009 | 73116260 | − |
| | | | Antisense) | | | | |
| SEQ ID NO. 334 | 2685954 | CPOX | Non-coding (UTR) | chr3 | 98298317 | 98298425 | − |
| SEQ ID NO. 335 | 2691756 | GOLGB1 | Coding | chr3 | 121417416 | 121417547 | − |
| SEQ ID NO. 336 | 2692111 | DTX3L | Non-coding (Exonic | chr3 | 122293046 | 122293265 | − |
| | | | Antisense) | | | | |
| SEQ ID NO. 337 | 2694407 | RPN1 | Coding | chr3 | 128345595 | 128345630 | − |
| SEQ ID NO. 338 | 2694648 | CNBP | Non-coding (UTR) | chr3 | 128888336 | 128888400 | − |
| SEQ ID NO. 339 | 2694649 | CNBP | Non-coding (UTR) | chr3 | 128888471 | 128888731 | − |
| SEQ ID NO. 340 | 2695413 | MRPL3 | Non-coding (UTR) | chr3 | 131181258 | 131181354 | − |
| SEQ ID NO. 341 | 2700507 | COMMD2 | Coding | chr3 | 149468465 | 149468497 | − |
| SEQ ID NO. 342 | 2700829 | SIAH2 | Non-coding (UTR) | chr3 | 150458934 | 150458997 | − |
| SEQ ID NO. 343 | 2701289 | MBNL1 | Non-coding (Intronic | chr3 | 152046480 | 152046743 | − |
| | | | Antisense) | | | | |
| SEQ ID NO. 344 | 2702329 | CCNL1 | Non-coding (UTR) | chr3 | 156865649 | 156865775 | − |
| SEQ ID NO. 345 | 2703240 | KPNA4 | Coding | chr3 | 160225956 | 160225986 | − |
| SEQ ID NO. 346 | 2704511 | MECOM | Non- | chr3 | 169031880 | 169032002 | − |
| | | | coding (Intronic) | | | | |
| SEQ ID NO. 347 | 2704898 | PHC3 | Non- | chr3 | 169808000 | 169808215 | − |
| | | | coding (Intronic) | | | | |
| SEQ ID NO. 348 | 2704935 | PHC3 | Coding | chr3 | 169889168 | 169889237 | − |
| SEQ ID NO. 349 | 2705001 | chr3−: 170112099-170112278 | Non- | chr3 | 170112099 | 170112278 | − |
| | | | coding (Intergenic) | | | | |
| SEQ ID NO. 350 | 2706322 | TBL1XR1 | Non-coding (UTR) | chr3 | 176739787 | 176740276 | − |
| SEQ ID NO. 351 | 2706324 | TBL1XR1 | Non-coding (UTR) | chr3 | 176741134 | 176741226 | − |
| SEQ ID NO. 352 | 2707780 | DCUN1D1 | Coding | chr3 | 182665046 | 182665074 | − |
| SEQ ID NO. 353 | 2708798 | RP11-110C15.4 | Non- | chr3 | 185135627 | 185135657 | − |
| | | | coding (ncTranscript) | | | | |
| SEQ ID NO. 354 | 2709489 | RFC4 | Coding | chr3 | 186507758 | 186507817 | − |
| SEQ ID NO. 355 | 2710214 | LPP | Non-coding (Exonic | chr3 | 188595052 | 188595263 | − |
| | | | Antisense) | | | | |
| SEQ ID NO. 356 | 2710217 | LPP | Non-coding (Exonic | chr3 | 188596212 | 188596304 | − |
| | | | Antisense) | | | | |
| SEQ ID NO. 357 | 2711689 | ATP13A3 | Coding | chr3 | 194169199 | 194169357 | − |
| SEQ ID NO. 358 | 2712424 | TNK2 | Non-coding (UTR) | chr3 | 195590243 | 195590284 | − |
| SEQ ID NO. 359 | 2713075 | NCBP2 | Non-coding (UTR) | chr3 | 196662298 | 196662359 | − |
| SEQ ID NO. 360 | 2713262 | DLG1 | Coding | chr3 | 196865154 | 196865258 | − |
| SEQ ID NO. 361 | 2713269 | DLG1 | Coding | chr3 | 196876625 | 196876656 | − |
| SEQ ID NO. 362 | 2714106 | PIGG | Non-coding (UTR) | chr4 | 533179 | 533289 | + |
| SEQ ID NO. 363 | 2715470 | RNF4 | Non-coding (UTR) | chr4 | 2517539 | 2517569 | + |
| SEQ ID NO. 364 | 2715717 | ADD1 | Non-coding (UTR) | chr4 | 2931743 | 2931773 | + |
| SEQ ID NO. 365 | 2720693 | SLIT2 | Non-coding (UTR) | chr4 | 20622128 | 20622155 | + |
| SEQ ID NO. 366 | 2724050 | KLF3 | Non-coding (UTR) | chr4 | 38700710 | 38702100 | + |
| SEQ ID NO. 367 | 2724565 | PABPC1P1 | Non- | chr4 | 39975424 | 39975454 | + |
| | | | coding (ncTranscript) | | | | |
| SEQ ID NO. 368 | 2726514 | OCIAD1 | Coding | chr4 | 48859230 | 48859373 | + |
| SEQ ID NO. 369 | 2727970 | EXOC1 | Non-coding (UTR) | chr4 | 56771127 | 56771164 | + |
| SEQ ID NO. 370 | 2730701 | MOBKL1A | Coding | chr4 | 71844873 | 71844981 | + |
| SEQ ID NO. 371 | 2731872 | USO1 | Coding | chr4 | 76722241 | 76722353 | + |
| SEQ ID NO. 372 | 2732403 | CCNG2 | Coding | chr4 | 78081887 | 78082066 | + |
| SEQ ID NO. 373 | 2735017 | SPARCL1 | Non-coding (Exonic | chr4 | 88394536 | 88394668 | + |
| | | | Antisense) | | | | |
| SEQ ID NO. 374 | 2736295 | SMARCAD1 | Coding | chr4 | 95198175 | 95198297 | + |
| SEQ ID NO. 375 | 2738963 | OSTC | Non-coding (UTR) | chr4 | 109588601 | 109588723 | + |
| SEQ ID NO. 376 | 2743194 | PGRMC2 | Non-coding (Exonic | chr4 | 129191337 | 129192099 | + |
| | | | Antisense) | | | | |
| SEQ ID NO. 377 | 2744632 | NAA15 | Coding | chr4 | 140282939 | 140283030 | + |
| SEQ ID NO. 378 | 2745687 | SMARCA5 | Coding | chr4 | 144465026 | 144465105 | + |
| SEQ ID NO. 379 | 2754300 | IRF2 | Non-coding (Exonic | chr4 | 185308983 | 185309251 | + |
| | | | Antisense) | | | | |
| SEQ ID NO. 380 | 2757037 | CTBP1 | Non-coding (UTR) | chr4 | 1205264 | 1205300 | − |
| SEQ ID NO. 381 | 2763813 | DHX15 | Non-coding (UTR) | chr4 | 24529196 | 24529350 | − |
| SEQ ID NO. 382 | 2766634 | PDS5A | Coding | chr4 | 39878690 | 39878768 | − |
| SEQ ID NO. 383 | 2770195 | AASDH | Non-coding (UTR) | chr4 | 57204490 | 57204521 | − |
| SEQ ID NO. 384 | 2770473 | IGFBP7, RP11- | Non-coding (UTR) | chr4 | 57897402 | 57897479 | − |
| | | 738E22.2 | | | | | |
| SEQ ID NO. 385 | 2771759 | UBA6 | Coding | chr4 | 68506869 | 68506938 | − |
| SEQ ID NO. 386 | 2773129 | ANKRD17 | Non- | chr4 | 74079054 | 74079088 | − |
| | | | coding (Intronic) | | | | |
| SEQ ID NO. 387 | 2774054 | SCARB2 | Non-coding (UTR) | chr4 | 77081059 | 77081265 | − |
| SEQ ID NO. 388 | 2775592 | HNRPDL | Coding | chr4 | 83350409 | 83350477 | − |
| SEQ ID NO. 389 | 2775758 | SEC31A | Non-coding (UTR) | chr4 | 83739819 | 83739846 | − |
| SEQ ID NO. 390 | 2776094 | FAM175A | Coding | chr4 | 84383985 | 84384015 | − |
| SEQ ID NO. 391 | 2777492 | FAM13A | Non-coding (UTR) | chr4 | 89647632 | 89647659 | − |
| SEQ ID NO. 392 | 2779488 | H2AFZ | Non-coding (UTR) | chr4 | 100869259 | 100869283 | − |
| SEQ ID NO. 393 | 2779639 | PPP3CA | Non-coding (UTR) | chr4 | 101944595 | 101944629 | − |
| SEQ ID NO. 394 | 2779995 | UBE2D3 | Non-coding (UTR) | chr4 | 103717151 | 103717282 | − |
| SEQ ID NO. 395 | 2780545 | PPA2 | Non-coding (UTR) | chr4 | 106290788 | 106290854 | − |
| SEQ ID NO. 396 | 2783475 | C4orf3 | Non-coding (UTR) | chr4 | 120218228 | 120218288 | − |
| SEQ ID NO. 397 | 2789554 | SH3D19 | Non-coding (UTR) | chr4 | 152096727 | 152096784 | − |
| SEQ ID NO. 398 | 2790573 | PLRG1 | Non-coding (UTR) | chr4 | 155457687 | 155457728 | − |
| SEQ ID NO. 399 | 2797449 | FAT1 | Coding | chr4 | 187560891 | 187560927 | − |
| SEQ ID NO. 400 | 2801338 | CTD-2001E22.2 | Non- | chr5 | 9550353 | 9550381 | + |
| | | | coding (ncTranscript) | | | | |
| SEQ ID NO. 401 | 2801556 | CCT5 | Non-coding (UTR) | chr5 | 10265042 | 10265108 | + |
| SEQ ID NO. 402 | 2801679 | MARCH6 | Coding | chr5 | 10433706 | 10433796 | + |
| SEQ ID NO. 403 | 2802506 | TRIO | Coding | chr5 | 14358400 | 14358435 | + |
| SEQ ID NO. 404 | 2805739 | RP11-36C20.1 | Non- | chr5 | 33162531 | 33162567 | + |
| | | | coding (ncTranscript) | | | | |
| SEQ ID NO. 405 | 2806859 | NIPBL | Coding | chr5 | 36984905 | 36985298 | + |
| SEQ ID NO. 406 | 2806912 | NIPBL | Coding | chr5 | 37045613 | 37045670 | + |
| SEQ ID NO. 407 | 2807658 | AC008810.1 | Non-coding (Exonic | chr5 | 40761189 | 40761495 | + |
| | | | Antisense) | | | | |
| SEQ ID NO. 408 | 2809146 | PELO | Non-coding (UTR) | chr5 | 52097805 | 52097849 | + |
| SEQ ID NO. 409 | 2810487 | GPBP1 | Coding | chr5 | 56531793 | 56531821 | + |
| SEQ ID NO. 410 | 2812307 | PPWD1 | Non-coding (UTR) | chr5 | 64883290 | 64883318 | + |
| SEQ ID NO. 411 | 2812500 | ERBB2IP | Coding | chr5 | 65374321 | 65374358 | + |
| SEQ ID NO. 412 | 2812573 | SREK1 | Coding | chr5 | 65460623 | 65460704 | + |
| SEQ ID NO. 413 | 2817404 | PAPD4 | Coding | chr5 | 78919159 | 78919265 | + |
| SEQ ID NO. 414 | 2817457 | PAPD4 | Non-coding (UTR) | chr5 | 78982421 | 78982452 | + |
| SEQ ID NO. 415 | 2818271 | RPS23 | Non-coding (Exonic | chr5 | 81573522 | 81573652 | + |
| | | | Antisense) | | | | |
| SEQ ID NO. 416 | 2818964 | COX7C | Non-coding (UTR) | chr5 | 85916491 | 85916564 | + |
| SEQ ID NO. 417 | 2822274 | PAM | Non-coding (UTR) | chr5 | 102365342 | 102365372 | + |
| SEQ ID NO. 418 | 2823066 | EFNA5 | Non-coding (Intronic | chr5 | 106974955 | 106974985 | + |
| | | | Antisense) | | | | |
| SEQ ID NO. 419 | 2823854 | WDR36 | Coding | chr5 | 110456789 | 110456839 | + |
| SEQ ID NO. 420 | 2824899 | AP3S1 | Non-coding (UTR) | chr5 | 115249432 | 115249610 | + |
| SEQ ID NO. 421 | 2828112 | HINT1 | Non-coding (Exonic | chr5 | 130495072 | 130495104 | + |
| | | | Antisense) | | | | |
| SEQ ID NO. 422 | 2828637 | RAD50 | Coding | chr5 | 131939052 | 131939156 | + |
| SEQ ID NO. 423 | 2828642 | RAD50 | Coding | chr5 | 131945000 | 131945077 | + |
| SEQ ID NO. 424 | 2829255 | SKP1, PPP2CA | Non-coding (Exonic | chr5 | 133533062 | 133533258 | + |
| | | | Antisense) | | | | |
| SEQ ID NO. 425 | 2829555 | C5orf24 | Non-coding (UTR) | chr5 | 134195299 | 134195382 | + |
| SEQ ID NO. 426 | 2829679 | CTC-203F4.1 | Non- | chr5 | 134670206 | 134670393 | + |
| | | | coding (Intronic) | | | | |
| SEQ ID NO. 427 | 2831231 | PAIP2 | Non-coding (UTR) | chr5 | 138704988 | 138705329 | + |
| SEQ ID NO. 428 | 2831557 | C5orf32 | Non-coding (UTR) | chr5 | 139623334 | 139623371 | + |
| SEQ ID NO. 429 | 2836687 | SAP30L | Non-coding (UTR) | chr5 | 153835600 | 153835667 | + |
| SEQ ID NO. 430 | 2840638 | NPM1 | Coding | chr5 | 170832306 | 170832330 | + |
| SEQ ID NO. 431 | 2843280 | TMED9 | Non-coding (UTR) | chr5 | 177023035 | 177023099 | + |
| SEQ ID NO. 432 | 2844248 | CANX | Non-coding (UTR) | chr5 | 179157573 | 179157623 | + |
| SEQ ID NO. 433 | 2844250 | CANX | Non-coding (UTR) | chr5 | 179157864 | 179157894 | + |
| SEQ ID NO. 434 | 2848258 | CCT5 | Non-coding (Exonic | chr5 | 10264858 | 10265070 | − |
| | | | Antisense) | | | | |
| SEQ ID NO. 435 | 2848472 | DAP | Non-coding (UTR) | chr5 | 10679387 | 10679458 | − |
| SEQ ID NO. 436 | 2851973 | DROSHA | Coding | chr5 | 31401461 | 31401522 | − |
| SEQ ID NO. 437 | 2854808 | RPL37 | Coding | chr5 | 40834283 | 40834312 | − |
| SEQ ID NO. 438 | 2854809 | RPL37 | Coding | chr5 | 40834313 | 40834356 | − |
| SEQ ID NO. 439 | 2855289 | SEPP1 | Non-coding (UTR) | chr5 | 42800001 | 42800083 | − |
| SEQ ID NO. 440 | 2855291 | SEPP1 | Non-coding (UTR) | chr5 | 42800241 | 42800298 | − |
| SEQ ID NO. 441 | 2855292 | SEPP1 | Non-coding (UTR) | chr5 | 42800506 | 42800717 | − |
| SEQ ID NO. 442 | 2857172 | DHX29 | Coding | chr5 | 54589903 | 54589952 | − |
| SEQ ID NO. 443 | 2862162 | TNPO1 | Non-coding (Exonic | chr5 | 72161442 | 72161545 | − |
| | | | Antisense) | | | | |
| SEQ ID NO. 444 | 2862377 | BTF3 | Non-coding (Exonic | chr5 | 72801222 | 72801281 | − |
| | | | Antisense) | | | | |
| SEQ ID NO. 445 | 2865063 | RPS23 | Non-coding (UTR) | chr5 | 81571901 | 81571927 | − |
| SEQ ID NO. 446 | 2868141 | ERAP1 | Non-coding (UTR) | chr5 | 96110219 | 96110633 | − |
| SEQ ID NO. 447 | 2877518 | HSPA9 | Coding | chr5 | 137893091 | 137893176 | − |
| SEQ ID NO. 448 | 2878261 | PFDN1 | Coding | chr5 | 139661064 | 139661115 | − |
| SEQ ID NO. 449 | 2882121 | SPARC | Non-coding (UTR) | chr5 | 151042046 | 151042264 | − |
| SEQ ID NO. 450 | 2888344 | RNF44 | Non-coding (UTR) | chr5 | 175953724 | 175953810 | − |
| SEQ ID NO. 451 | 2893822 | DSP | Coding | chr6 | 7578699 | 7578774 | + |
| SEQ ID NO. 452 | 2893942 | TXNDC5, MUTED | Non-coding (Exonic | chr6 | 7881819 | 7881943 | + |
| | | | Antisense) | | | | |
| SEQ ID NO. 453 | 2896978 | FAM8A1 | Non-coding (UTR) | chr6 | 17611895 | 17611944 | + |
| SEQ ID NO. 454 | 2898531 | ALDH5A1 | Non-coding (UTR) | chr6 | 24537390 | 24537418 | + |
| SEQ ID NO. 455 | 2902010 | DDR1 | Non-coding (UTR) | chr6 | 30867773 | 30867802 | + |
| SEQ ID NO. 456 | 2902011 | DDR1 | Coding | chr6 | 30867849 | 30867902 | + |
| SEQ ID NO. 457 | 2905137 | SRSF3 | Non-coding (UTR) | chr6 | 36570141 | 36570256 | + |
| SEQ ID NO. 458 | 2906968 | PRICKLE4, TOMM6 | Non-coding (UTR) | chr6 | 41757493 | 41757600 | + |
| SEQ ID NO. 459 | 2908048 | TJAP1 | Non-coding (UTR) | chr6 | 43474171 | 43474248 | + |
| SEQ ID NO. 460 | 2909471 | CD2AP | Coding | chr6 | 47580215 | 47580271 | + |
| SEQ ID NO. 461 | 2910489 | FBXO9 | Coding | chr6 | 52938339 | 52938411 | + |
| SEQ ID NO. 462 | 2914002 | SENP6 | Non- | chr6 | 76345055 | 76345281 | + |
| | | | coding (Intronic) | | | | |
| SEQ ID NO. 463 | 2914107 | MYO6 | Coding | chr6 | 76576694 | 76576822 | + |
| SEQ ID NO. 464 | 2916383 | SLC35A1 | Non-coding (UTR) | chr6 | 88222005 | 88222043 | + |
| SEQ ID NO. 465 | 2916976 | CASP8AP2 | Coding | chr6 | 90565189 | 90565242 | + |
| SEQ ID NO. 466 | 2918543 | SFRS18 | Non-coding (Exonic | chr6 | 99846655 | 99846962 | + |
| | | | Antisense) | | | | |
| SEQ ID NO. 467 | 2918547 | SFRS18 | Non-coding (Exonic | chr6 | 99848935 | 99849230 | + |
| | | | Antisense) | | | | |
| SEQ ID NO. 468 | 2922232 | MARCKS | Non-coding (UTR) | chr6 | 114183054 | 114183145 | + |
| SEQ ID NO. 469 | 2922235 | MARCKS | Non-coding (UTR) | chr6 | 114184550 | 114184627 | + |
| SEQ ID NO. 470 | 2926222 | RPS12 | Coding | chr6 | 133138646 | 133138675 | + |
| SEQ ID NO. 471 | 2929946 | STXBP5 | Non- | chr6 | 147669278 | 147669305 | + |
| | | | coding (Intronic) | | | | |
| SEQ ID NO. 472 | 2930652 | TAB2 | Non-coding (UTR) | chr6 | 149732661 | 149732705 | + |
| SEQ ID NO. 473 | 2932399 | SCAF8 | Coding | chr6 | 155126504 | 155126588 | + |
| SEQ ID NO. 474 | 2932428 | SCAF8 | Non-coding (UTR) | chr6 | 155155164 | 155155191 | + |
| SEQ ID NO. 475 | 2933660 | TMEM181 | Coding | chr6 | 159001973 | 159002010 | + |
| SEQ ID NO. 476 | 2936963 | MLLT4 | Coding | chr6 | 168370531 | 168370582 | + |
| SEQ ID NO. 477 | 2940827 | TXNDC5, MUTED | Non-coding (UTR) | chr6 | 7881828 | 7882072 | − |
| SEQ ID NO. 478 | 2942512 | RANBP9 | Coding | chr6 | 13625915 | 13625996 | − |
| SEQ ID NO. 479 | 2944070 | DEK | Non-coding (UTR) | chr6 | 18224463 | 18224675 | − |
| SEQ ID NO. 480 | 2944963 | SOX4 | Non-coding (Exonic | chr6 | 21598569 | 21598785 | − |
| | | | Antisense) | | | | |
| SEQ ID NO. 481 | 2946479 | HMGN4 | Non-coding (Exonic | chr6 | 26546937 | 26547116 | − |
| | | | Antisense) | | | | |
| SEQ ID NO. 482 | 2947592 | TRIM27 | Coding | chr6 | 28876750 | 28876789 | − |
| SEQ ID NO. 483 | 2948592 | TUBB | Non-coding (Exonic | chr6 | 30691418 | 30691448 | − |
| | | | Antisense) | | | | |
| SEQ ID NO. 484 | 2948596 | FLOT1 | Non-coding (UTR) | chr6 | 30695520 | 30695552 | − |
| SEQ ID NO. 485 | 2949053 | DDX39B | Non- | chr6 | 31500977 | 31501368 | − |
| | | | coding (ncTranscript) | | | | |
| SEQ ID NO. 486 | 2949061 | XXbac- | Coding | chr6 | 31504283 | 31504313 | − |
| | | BPG296P20.16.DDX39B | | | | | |
| SEQ ID NO. 487 | 2951060 | C6orf1 | Non-coding (UTR) | chr6 | 34214199 | 34214278 | − |
| SEQ ID NO. 488 | 2956077 | TNFRSF21 | Coding | chr6 | 47252104 | 47252155 | − |
| SEQ ID NO. 489 | 2958402 | DST | Coding | chr6 | 56394805 | 56394917 | − |
| SEQ ID NO. 490 | 2961302 | COX7A2 | Coding | chr6 | 75947554 | 75947636 | − |
| SEQ ID NO. 491 | 2966293 | SFRS18 | Non-coding (UTR) | chr6 | 99864226 | 99864304 | − |
| SEQ ID NO. 492 | 2966378 | CCNC | Non-coding (UTR) | chr6 | 99990291 | 99990460 | − |
| SEQ ID NO. 493 | 2967164 | HACE1 | Non-coding (UTR) | chr6 | 105176560 | 105176777 | − |
| SEQ ID NO. 494 | 2967323 | PREP | Non-coding (UTR) | chr6 | 105725610 | 105725643 | − |
| SEQ ID NO. 495 | 2968257 | SNX3 | Coding | chr6 | 108581964 | 108581997 | − |
| SEQ ID NO. 496 | 2968653 | SESN1 | Non-coding (UTR) | chr6 | 109307670 | 109307727 | − |
| SEQ ID NO. 497 | 2974195 | MED23 | Coding | chr6 | 131907897 | 131907950 | − |
| SEQ ID NO. 498 | 2974332 | CTGF | Non-coding (UTR) | chr6 | 132269390 | 132269425 | − |
| SEQ ID NO. 499 | 2975686 | BCLAF1 | Non-coding (UTR) | chr6 | 136580814 | 136581178 | − |
| SEQ ID NO. 500 | 2975709 | BCLAF1 | Coding | chr6 | 136594260 | 136594296 | − |
| SEQ ID NO. 501 | 2976368 | PERP | Non-coding (UTR) | chr6 | 138412032 | 138412084 | − |
| SEQ ID NO. 502 | 2977521 | FUCA2 | Non-coding (UTR) | chr6 | 143816625 | 143816660 | − |
| SEQ ID NO. 503 | 2981918 | EZR | Non-coding (UTR) | chr6 | 159187437 | 159187559 | − |
| SEQ ID NO. 504 | 2982323 | SOD2 | Non-coding (UTR) | chr6 | 160100449 | 160100520 | − |
| SEQ ID NO. 505 | 2982385 | TCP1 | Coding | chr6 | 160200252 | 160200282 | − |
| SEQ ID NO. 506 | 2984576 | SFT2D1 | Non-coding (UTR) | chr6 | 166733526 | 166733682 | − |
| SEQ ID NO. 507 | 2985813 | THBS2 | Non-coding (UTR) | chr6 | 169617449 | 169617654 | − |
| SEQ ID NO. 508 | 2985965 | WDR27 | Non-coding (UTR) | chr6 | 169857317 | 169857350 | − |
| SEQ ID NO. 509 | 2988895 | AIMP2 | Non-coding (UTR) | chr7 | 6063363 | 6063418 | + |
| SEQ ID NO. 510 | 2988896 | AIMP2 | Non-coding (UTR) | chr7 | 6063425 | 6063456 | + |
| SEQ ID NO. 511 | 2989720 | AC007128.1, AC007009.1 | Non- | chr7 | 8302393 | 8302437 | + |
| | | | coding (ncTranscript) | | | | |
| SEQ ID NO. 512 | 2991248 | AHR | Coding | chr7 | 17373588 | 17373738 | + |
| SEQ ID NO. 513 | 2991263 | AHR | Non-coding (UTR) | chr7 | 17382754 | 17383243 | + |
| SEQ ID NO. 514 | 2991264 | AHR | Non-coding (UTR) | chr7 | 17383478 | 17384186 | + |
| SEQ ID NO. 515 | 2993622 | HNRNPA2B1 | Non-coding (Exonic | chr7 | 26230040 | 26230071 | + |
| | | | Antisense) | | | | |
| SEQ ID NO. 516 | 2993644 | CBX3 | Non-coding (UTR) | chr7 | 26242591 | 26242618 | + |
| SEQ ID NO. 517 | 2993659 | CBX3 | Non-coding (UTR) | chr7 | 26252158 | 26252837 | + |
| SEQ ID NO. 518 | 2994371 | TAX1BP1 | Non- | chr7 | 27841295 | 27841322 | + |
| | | | coding (Intronic) | | | | |
| SEQ ID NO. 519 | 2995598 | AQP1 | Non-coding (UTR) | chr7 | 30965061 | 30965090 | + |
| SEQ ID NO. 520 | 2997143 | SEPT7 | Coding | chr7 | 35930330 | 35930354 | + |
| SEQ ID NO. 521 | 3002667 | EGFR | Non- | chr7 | 55111601 | 55111721 | + |
| | | | coding (ncTranscript) | | | | |
| SEQ ID NO. 522 | 3003180 | GBAS | Non-coding (UTR) | chr7 | 56067815 | 56067843 | + |
| SEQ ID NO. 523 | 3004652 | chr7+: 64171405-64171435 | Non- | chr7 | 64171405 | 64171435 | + |
| | | | coding (Intergenic) | | | | |
| SEQ ID NO. 524 | 3011652 | EEF1A1P28 | Non- | chr7 | 88269706 | 88269736 | + |
| | | | coding (ncTranscript) | | | | |
| SEQ ID NO. 525 | 3011846 | STEAP1 | Non-coding (UTR) | chr7 | 89794089 | 89794119 | + |
| SEQ ID NO. 526 | 3012405 | AKAP9 | Coding | chr7 | 91624943 | 91625038 | + |
| SEQ ID NO. 527 | 3012428 | AKAP9 | Coding | chr7 | 91670017 | 91670138 | + |
| SEQ ID NO. 528 | 3012438 | AKAP9 | Coding | chr7 | 91682056 | 91682090 | + |
| SEQ ID NO. 529 | 3012452 | AKAP9 | Coding | chr7 | 91700231 | 91700287 | + |
| SEQ ID NO. 530 | 3012453 | AKAP9 | Coding | chr7 | 91700288 | 91700319 | + |
| SEQ ID NO. 531 | 3013190 | CASD1 | Coding | chr7 | 94147516 | 94147581 | + |
| SEQ ID NO. 532 | 3013468 | PON2 | Non-coding (Intronic | chr7 | 95042749 | 95042946 | + |
| | | | Antisense) | | | | |
| SEQ ID NO. 533 | 3013685 | SLC25A13 | Non-coding (Exonic | chr7 | 95749570 | 95749793 | + |
| | | | Antisense) | | | | |
| SEQ ID NO. 534 | 3014422 | TRRAP | Coding | chr7 | 98493399 | 98493443 | + |
| SEQ ID NO. 535 | 3015985 | SRRT | Coding | chr7 | 100486179 | 100486273 | + |
| SEQ ID NO. 536 | 3017629 | MLL5 | Coding | chr7 | 104746413 | 104746442 | + |
| SEQ ID NO. 537 | 3017826 | RINT1 | Non-coding (UTR) | chr7 | 105208082 | 105208113 | + |
| SEQ ID NO. 538 | 3021760 | WASL | Non-coding (Exonic | chr7 | 123324045 | 123324364 | + |
| | | | Antisense) | | | | |
| SEQ ID NO. 539 | 3022682 | SND1 | Non-coding (UTR) | chr7 | 127732585 | 127732630 | + |
| SEQ ID NO. 540 | 3027555 | NDUFB2 | Non-coding (UTR) | chr7 | 140406369 | 140406414 | + |
| SEQ ID NO. 541 | 3027926 | SSBP1 | Coding | chr7 | 141443718 | 141443782 | + |
| SEQ ID NO. 542 | 3027936 | SSBP1 | Non-coding (UTR) | chr7 | 141450185 | 141450241 | + |
| SEQ ID NO. 543 | 3037391 | KDELR2 | Non-coding (UTR) | chr7 | 6502522 | 6502596 | − |
| SEQ ID NO. 544 | 3037406 | KDELR2 | Coding | chr7 | 6513851 | 6513887 | − |
| SEQ ID NO. 545 | 3037950 | RPA3 | Non-coding (UTR) | chr7 | 7676584 | 7676627 | − |
| SEQ ID NO. 546 | 3039980 | AHR | Non-coding (Exonic | chr7 | 17382756 | 17383093 | − |
| | | | Antisense) | | | | |
| SEQ ID NO. 547 | 3039983 | AHR | Non-coding (Exonic | chr7 | 17384068 | 17384200 | − |
| | | | Antisense) | | | | |
| SEQ ID NO. 548 | 3040116 | SNX13 | Coding | chr7 | 17915324 | 17915397 | − |
| SEQ ID NO. 549 | 3041262 | TOMM7 | Non-coding (UTR) | chr7 | 22852644 | 22852678 | − |
| SEQ ID NO. 550 | 3042004 | CYCS | Non-coding (UTR) | chr7 | 25162509 | 25162708 | − |
| SEQ ID NO. 551 | 3042424 | HNRNPA2B1 | Non-coding (UTR) | chr7 | 26229786 | 26229921 | − |
| SEQ ID NO. 552 | 3042426 | HNRNPA2B1 | Non-coding (UTR) | chr7 | 26230047 | 26230074 | − |
| SEQ ID NO. 553 | 3042429 | HNRNPA2B1 | Non-coding (UTR) | chr7 | 26230678 | 26230737 | − |
| SEQ ID NO. 554 | 3042430 | HNRNPA2B1 | Non-coding (UTR) | chr7 | 26230767 | 26230996 | − |
| SEQ ID NO. 555 | 3042437 | HNRNPA2B1 | Non-coding (UTR) | chr7 | 26231796 | 26231958 | − |
| SEQ ID NO. 556 | 3042453 | HNRNPA2B1 | Coding | chr7 | 26236208 | 26236278 | − |
| SEQ ID NO. 557 | 3048780 | TMED4 | Non-coding (UTR) | chr7 | 44617913 | 44617943 | − |
| SEQ ID NO. 558 | 3048870 | H2AFV | Non-coding (UTR) | chr7 | 44866413 | 44866768 | − |
| SEQ ID NO. 559 | 3053384 | chr7−: 64435274-64435303 | Non- | chr7 | 64435274 | 64435303 | − |
| | | | coding (Intergenic) | | | | |
| SEQ ID NO. 560 | 3056395 | EIF4H | Non-coding (Exonic | chr7 | 73611210 | 73611234 | − |
| | | | Antisense) | | | | |
| SEQ ID NO. 561 | 3060052 | DMTF1 | Non-coding (Exonic | chr7 | 86824937 | 86825415 | − |
| | | | Antisense) | | | | |
| SEQ ID NO. 562 | 3061054 | AC000120.7, KRIT1 | Non-coding (UTR) | chr7 | 91829356 | 91829670 | − |
| SEQ ID NO. 563 | 3061144 | ANKIB1 | Non-coding (Exonic | chr7 | 92029299 | 92029375 | − |
| | | | Antisense) | | | | |
| SEQ ID NO. 564 | 3061758 | COL1A2 | Non-coding (Exonic | chr7 | 94058608 | 94058723 | − |
| | | | Antisense) | | | | |
| SEQ ID NO. 565 | 3062023 | PON2 | Non- | chr7 | 95041837 | 95042319 | − |
| | | | coding (ncTranscript) | | | | |
| SEQ ID NO. 566 | 3064354 | SRRT | Non-coding (Exonic | chr7 | 100486138 | 100486258 | − |
| | | | Antisense) | | | | |
| SEQ ID NO. 567 | 3075645 | chr7−: 138912234-138912268 | Non- | chr7 | 138912234 | 138912268 | − |
| | | | coding (Intergenic) | | | | |
| SEQ ID NO. 568 | 3076355 | NDUFB2 | Non-coding (Intronic | chr7 | 140419154 | 140419193 | − |
| | | | Antisense) | | | | |
| SEQ ID NO. 569 | 3076393 | BRAF | Coding | chr7 | 140501240 | 140501268 | − |
| SEQ ID NO. 570 | 3076490 | MRPS33 | Non-coding (UTR) | chr7 | 140705974 | 140706021 | − |
| SEQ ID NO. 571 | 3081625 | LMBR1 | Non-coding (UTR) | chr7 | 156475639 | 156475683 | − |
| SEQ ID NO. 572 | 3087828 | PCM1 | Coding | chr8 | 17810521 | 17810685 | + |
| SEQ ID NO. 573 | 3087930 | ASAH1 | Non-coding (Exonic | chr8 | 17924332 | 17924610 | + |
| | | | Antisense) | | | | |
| SEQ ID NO. 574 | 3088570 | ATP6V1B2 | Non-coding (UTR) | chr8 | 20079154 | 20079183 | + |
| SEQ ID NO. 575 | 3092505 | RBPMS | Non-coding (UTR) | chr8 | 30404975 | 30405013 | + |
| SEQ ID NO. 576 | 3094309 | PROSC | Non-coding (UTR) | chr8 | 37637241 | 37637268 | + |
| SEQ ID NO. 577 | 3096162 | IKBKB | Non-coding (UTR) | chr8 | 42189466 | 42189790 | + |
| SEQ ID NO. 578 | 3096409 | HOOK3 | Coding | chr8 | 42862996 | 42863063 | + |
| SEQ ID NO. 579 | 3096979 | KIAA0146 | Coding | chr8 | 48309034 | 48309125 | + |
| SEQ ID NO. 580 | 3099780 | SDCBP | Non-coding (UTR) | chr8 | 59495253 | 59495349 | + |
| SEQ ID NO. 581 | 3100230 | RAB2A | Non-coding (UTR) | chr8 | 61533537 | 61533612 | + |
| SEQ ID NO. 582 | 3107638 | DPY19L4 | Coding | chr8 | 95795948 | 95795975 | + |
| SEQ ID NO. 583 | 3108472 | MTDH | Coding | chr8 | 98731359 | 98731417 | + |
| SEQ ID NO. 584 | 3108501 | LAPTM4B | Coding | chr8 | 98828334 | 98828363 | + |
| SEQ ID NO. 585 | 3108938 | VPS13B | Coding | chr8 | 100160128 | 100160223 | + |
| SEQ ID NO. 586 | 3111090 | AC090579.1 | Coding | chr8 | 107719006 | 107719178 | + |
| SEQ ID NO. 587 | 3111116 | AC090579.1 | Non-coding (UTR) | chr8 | 107764863 | 107764893 | + |
| SEQ ID NO. 588 | 3112517 | EIF3H | Non-coding (Exonic | chr8 | 117657108 | 117657268 | + |
| | | | Antisense) | | | | |
| SEQ ID NO. 589 | 3114370 | FAM91A1 | Coding | chr8 | 124792239 | 124792277 | + |
| SEQ ID NO. 590 | 3114664 | NDUFB9 | Non-coding (UTR) | chr8 | 125562138 | 125562181 | + |
| SEQ ID NO. 591 | 3124536 | FDFT1 | Non-coding (Exonic | chr8 | 11696124 | 11696400 | − |
| | | | Antisense) | | | | |
| SEQ ID NO. 592 | 3126094 | ASAH1 | Non-coding (UTR) | chr8 | 17913971 | 17914068 | − |
| SEQ ID NO. 593 | 3128680 | SDADIP1 | Non- | chr8 | 26238141 | 26238174 | − |
| | | | coding (ncTranscript) | | | | |
| SEQ ID NO. 594 | 3129955 | TMEM66 | Coding | chr8 | 29923504 | 29923543 | − |
| SEQ ID NO. 595 | 3131973 | WHSC1L1 | Coding | chr8 | 38205449 | 38205479 | − |
| SEQ ID NO. 596 | 3132336 | TM2D2 | Non-coding (UTR) | chr8 | 38848290 | 38848319 | − |
| SEQ ID NO. 597 | 3133528 | RP11- | Non-coding (Exonic | chr8 | 42882619 | 42882650 | − |
| | | 598P20.5, HOOK3 | Antisense) | | | | |
| SEQ ID NO. 598 | 3134184 | PRKDC | Coding | chr8 | 48852116 | 48852256 | − |
| SEQ ID NO. 599 | 3136352 | RP11-17A4.1 | Non-coding (Exonic | chr8 | 57501108 | 57501163 | − |
| | | | Antisense) | | | | |
| SEQ ID NO. 600 | 3137586 | ASPH | Non-coding (UTR) | chr8 | 62537157 | 62537642 | − |
| SEQ ID NO. 601 | 3138886 | SNHG6 | Non- | chr8 | 67834852 | 67834955 | − |
| | | | coding (ncTranscript) | | | | |
| SEQ ID NO. 602 | 3138980 | COPS5 | Non-coding (UTR) | chr8 | 67955322 | 67955454 | − |
| SEQ ID NO. 603 | 3139053 | ARFGEF1 | Coding | chr8 | 68112673 | 68112748 | − |
| SEQ ID NO. 604 | 3139926 | TRAM1 | Coding | chr8 | 71510160 | 71510244 | − |
| SEQ ID NO. 605 | 3141863 | TPD52 | Non-coding (UTR) | chr8 | 80948831 | 80948859 | − |
| SEQ ID NO. 606 | 3145023 | KIAA1429 | Non-coding (UTR) | chr8 | 95500766 | 95500848 | − |
| SEQ ID NO. 607 | 3145956 | RPL30 | Coding | chr8 | 99054030 | 99054078 | − |
| SEQ ID NO. 608 | 3145966 | RPL30 | Coding | chr8 | 99057186 | 99057310 | − |
| SEQ ID NO. 609 | 3146538 | POLR2K | Non-coding (Exonic | chr8 | 101166096 | 101166165 | − |
| | | | Antisense) | | | | |
| SEQ ID NO. 610 | 3146566 | RNF19A | Non-coding (UTR) | chr8 | 101269317 | 101269521 | − |
| SEQ ID NO. 611 | 3146788 | PABPC1 | Non-coding (UTR) | chr8 | 101715241 | 101715454 | − |
| SEQ ID NO. 612 | 3146799 | PABPC1 | Coding | chr8 | 101721421 | 101721445 | − |
| SEQ ID NO. 613 | 3147328 | UBR5 | Coding | chr8 | 103269860 | 103269932 | − |
| SEQ ID NO. 614 | 3148628 | EIF3E | Coding | chr8 | 109240515 | 109240544 | − |
| SEQ ID NO. 615 | 3149768 | EIF3H | Non-coding (UTR) | chr8 | 117657087 | 117657221 | − |
| SEQ ID NO. 616 | 3149773 | EIF3H | Coding | chr8 | 117661058 | 117661159 | − |
| SEQ ID NO. 617 | 3149864 | RAD21 | Coding | chr8 | 117868898 | 117868928 | − |
| SEQ ID NO. 618 | 3151480 | C8orf76 | Non-coding (UTR) | chr8 | 124232257 | 124232285 | − |
| SEQ ID NO. 619 | 3152560 | FAM84B | Non-coding (UTR) | chr8 | 127564856 | 127565021 | − |
| SEQ ID NO. 620 | 3153532 | ASAP1 | Coding | chr8 | 131199487 | 131199516 | − |
| SEQ ID NO. 621 | 3157388 | TOP1MT | Non-coding (UTR) | chr8 | 144391539 | 144391610 | − |
| SEQ ID NO. 622 | 3157461 | ZC3H3 | Non-coding (UTR) | chr8 | 144519831 | 144519864 | − |
| SEQ ID NO. 623 | 3157723 | FAM83H | Non-coding (UTR) | chr8 | 144806118 | 144806196 | − |
| SEQ ID NO. 624 | 3157847 | NRBP2 | Non-coding (UTR) | chr8 | 144915923 | 144916015 | − |
| SEQ ID NO. 625 | 3164299 | DENND4C | Non-coding (UTR) | chr9 | 19373469 | 19374005 | + |
| SEQ ID NO. 626 | 3165138 | RP11-370B11.1 | Non- | chr9 | 22748070 | 22748104 | + |
| | | | coding (ncTranscript) | | | | |
| SEQ ID NO. 627 | 3165799 | IFT74 | Coding | chr9 | 27018647 | 27018685 | + |
| SEQ ID NO. 628 | 3166029 | chr9+: 27943648-27943678 | Non- | chr9 | 27943648 | 27943678 | + |
| | | | coding (Intergenic) | | | | |
| SEQ ID NO. 629 | 3166735 | DNAJA1 | Coding | chr9 | 33038903 | 33038998 | + |
| SEQ ID NO. 630 | 3167990 | RP11-156G14.6 | Non- | chr9 | 35592905 | 35592943 | + |
| | | | coding (ncTranscript) | | | | |
| SEQ ID NO. 631 | 3168127 | GBA2 | Non-coding (Exonic | chr9 | 35738843 | 35738870 | + |
| | | | Antisense) | | | | |
| SEQ ID NO. 632 | 3169011 | FRMPD1 | Coding | chr9 | 37735573 | 37735720 | + |
| SEQ ID NO. 633 | 3174268 | SMC5 | Coding | chr9 | 72967152 | 72967246 | + |
| SEQ ID NO. 634 | 3174441 | C9orf85 | Non-coding (UTR) | chr9 | 74588315 | 74588354 | + |
| SEQ ID NO. 635 | 3175632 | VPS13A | Coding | chr9 | 79895103 | 79895154 | + |
| SEQ ID NO. 636 | 3175657 | VPS13A | Coding | chr9 | 79936422 | 79936637 | + |
| SEQ ID NO. 637 | 3178635 | SECISBP2 | Coding | chr9 | 91949555 | 91949616 | + |
| SEQ ID NO. 638 | 3183178 | SLC44A1 | Non-coding (UTR) | chr9 | 108152027 | 108152059 | + |
| SEQ ID NO. 639 | 3190449 | CERCAM, AL359091.1 | Coding | chr9 | 131199594 | 131199623 | + |
| SEQ ID NO. 640 | 3192674 | GTF3C5 | Coding | chr9 | 135929237 | 135929328 | + |
| SEQ ID NO. 641 | 3197644 | ERMP1 | Non-coding (UTR) | chr9 | 5784578 | 5784615 | − |
| SEQ ID NO. 642 | 3199457 | ZDHHC21 | Non-coding (UTR) | chr9 | 14617270 | 14617297 | − |
| SEQ ID NO. 643 | 3199485 | ZDHHC21, AL390732.1 | Non- | chr9 | 14679025 | 14679408 | − |
| | | | coding (Intronic) | | | | |
| SEQ ID NO. 644 | 3200638 | HAUS6 | Coding | chr9 | 19087118 | 19087151 | − |
| SEQ ID NO. 645 | 3200711 | DENND4C | Non-coding (Exonic | chr9 | 19373580 | 19373641 | − |
| | | | Antisense) | | | | |
| SEQ ID NO. 646 | 3200718 | RPS6 | Coding | chr9 | 19376535 | 19376564 | − |
| SEQ ID NO. 647 | 3200725 | RPS6 | Coding | chr9 | 19378719 | 19378755 | − |
| SEQ ID NO. 648 | 3203822 | UBAP2 | Coding | chr9 | 33996241 | 33996307 | − |
| SEQ ID NO. 649 | 3205039 | RP11-327L3.5 | Non- | chr9 | 35971885 | 35971912 | − |
| | | | coding (ncTranscript) | | | | |
| SEQ ID NO. 650 | 3205424 | ZCCHC7 | Non-coding (Intronic | chr9 | 37193836 | 37193865 | − |
| | | | Antisense) | | | | |
| SEQ ID NO. 651 | 3212146 | UBQLN1 | Non-coding (UTR) | chr9 | 86275826 | 86276211 | − |
| SEQ ID NO. 652 | 3212163 | UBQLN1 | Coding | chr9 | 86294773 | 86294919 | − |
| SEQ ID NO. 653 | 3214671 | IARS | Coding | chr9 | 94973097 | 94973125 | − |
| SEQ ID NO. 654 | 3214699 | IARS | Coding | chr9 | 95012528 | 95012559 | − |
| SEQ ID NO. 655 | 3219683 | C9orf5 | Non-coding (UTR) | chr9 | 111777496 | 111777531 | − |
| SEQ ID NO. 656 | 3219711 | C9orf5 | Coding | chr9 | 111822639 | 111822721 | − |
| SEQ ID NO. 657 | 3219891 | PTPN3 | Non-coding (UTR) | chr9 | 112140545 | 112140610 | − |
| SEQ ID NO. 658 | 3220159 | TXN | Non-coding (UTR) | chr9 | 113006334 | 113006361 | − |
| SEQ ID NO. 659 | 3222054 | ATP6V1G1 | Non-coding (Exonic | chr9 | 117360479 | 117360624 | − |
| | | | Antisense) | | | | |
| SEQ ID NO. 660 | 3222984 | RP11-442A13.1 | Non- | chr9 | 120845349 | 120845391 | − |
| | | | coding (ncTranscript) | | | | |
| SEQ ID NO. 661 | 3223876 | RAB14 | Non-coding (UTR) | chr9 | 123943110 | 123943464 | − |
| SEQ ID NO. 662 | 3225580 | PBX3 | Non-coding (Intronic | chr9 | 128526259 | 128526303 | − |
| | | | Antisense) | | | | |
| SEQ ID NO. 663 | 3226470 | GOLGA2 | Coding | chr9 | 131028567 | 131028595 | − |
| SEQ ID NO. 664 | 3227646 | UCK1 | Non-coding (UTR) | chr9 | 134399202 | 134399229 | − |
| SEQ ID NO. 665 | 3233387 | C10orf18 | Non-coding (UTR) | chr10 | 5805626 | 5805670 | + |
| SEQ ID NO. 666 | 3234175 | ATP5C1 | Non-coding (UTR) | chr10 | 7849648 | 7849732 | + |
| SEQ ID NO. 667 | 3235500 | CDC123 | Coding | chr10 | 12292460 | 12292553 | + |
| SEQ ID NO. 668 | 3237000 | VIM | Non-coding (UTR) | chr10 | 17279281 | 17279308 | + |
| SEQ ID NO. 669 | 3237001 | VIM | Non-coding (UTR) | chr10 | 17279361 | 17279398 | + |
| SEQ ID NO. 670 | 3239927 | ABI1 | Non-coding (Exonic | chr10 | 27036907 | 27037562 | + |
| | | | Antisense) | | | | |
| SEQ ID NO. 671 | 3239982 | YME1L1 | Non-coding (Exonic | chr10 | 27399495 | 27399646 | + |
| | | | Antisense) | | | | |
| SEQ ID NO. 672 | 3240135 | RAB18 | Non-coding (UTR) | chr10 | 27828060 | 27828089 | + |
| SEQ ID NO. 673 | 3240409 | WAC | Coding | chr10 | 28899657 | 28899737 | + |
| SEQ ID NO. 674 | 3240425 | WAC | Non-coding (UTR) | chr10 | 28909409 | 28909439 | + |
| SEQ ID NO. 675 | 3241499 | KIF5B | Non-coding (Exonic | chr10 | 32299937 | 32300224 | + |
| | | | Antisense) | | | | |
| SEQ ID NO. 676 | 3241506 | KIF5B | Non-coding (Exonic | chr10 | 32321635 | 32321697 | + |
| | | | Antisense) | | | | |
| SEQ ID NO. 677 | 3243292 | HSD17B7P2 | Non- | chr10 | 38667320 | 38667344 | + |
| | | | coding (ncTranscript) | | | | |
| SEQ ID NO. 678 | 3250201 | VPS26A | Non-coding (UTR) | chr10 | 70932578 | 70932609 | + |
| SEQ ID NO. 679 | 3251409 | DDIT4 | Non-coding (UTR) | chr10 | 74035646 | 74035717 | + |
| SEQ ID NO. 680 | 3252608 | VDAC2 | Non-coding (UTR) | chr10 | 76990904 | 76990928 | + |
| SEQ ID NO. 681 | 3253445 | RPS24 | Coding | chr10 | 79795283 | 79795370 | + |
| SEQ ID NO. 682 | 3253863 | ZMIZ1 | Non-coding (UTR) | chr10 | 81076140 | 81076245 | + |
| SEQ ID NO. 683 | 3255269 | GHITM | Non-coding (UTR) | chr10 | 85912224 | 85912254 | + |
| SEQ ID NO. 684 | 3257404 | KIF20B | Non-coding (UTR) | chr10 | 91534618 | 91534656 | + |
| SEQ ID NO. 685 | 3258021 | BTAF1 | Non-coding (UTR) | chr10 | 93790008 | 93790053 | + |
| SEQ ID NO. 686 | 3259678 | LCOR | Non-coding (UTR) | chr10 | 98719067 | 98719119 | + |
| SEQ ID NO. 687 | 3261550 | GBF1 | Coding | chr10 | 104019825 | 104019856 | + |
| SEQ ID NO. 688 | 3262470 | SLK | Coding | chr10 | 105778558 | 105778664 | + |
| SEQ ID NO. 689 | 3263804 | SMC3 | Coding | chr10 | 112342321 | 112342395 | + |
| SEQ ID NO. 690 | 3264697 | TCF7L2 | Coding | chr10 | 114911484 | 114911512 | + |
| SEQ ID NO. 691 | 3264726 | TCF7L2 | Non-coding (UTR) | chr10 | 114927386 | 114927419 | + |
| SEQ ID NO. 692 | 3275175 | GDI2 | Coding | chr10 | 5842593 | 5842621 | − |
| SEQ ID NO. 693 | 3276455 | KIN | Coding | chr10 | 7822227 | 7822261 | − |
| SEQ ID NO. 694 | 3277709 | UPF2 | Coding | chr10 | 12041905 | 12041992 | − |
| SEQ ID NO. 695 | 3279906 | VIM | Non-coding (Exonic | chr10 | 17279228 | 17279424 | − |
| | | | Antisense) | | | | |
| SEQ ID NO. 696 | 3280961 | DNAJC1 | Non- | chr10 | 22268411 | 22268452 | − |
| | | | coding (Intronic) | | | | |
| SEQ ID NO. 697 | 3282022 | AB11 | Non-coding (UTR) | chr10 | 27037123 | 27037168 | − |
| SEQ ID NO. 698 | 3282170 | ANKRD26 | Coding | chr10 | 27342283 | 27342313 | − |
| SEQ ID NO. 699 | 3282215 | YME1L1 | Non-coding (UTR) | chr10 | 27399405 | 27399460 | − |
| SEQ ID NO. 700 | 3282220 | YME1L1 | Non-coding (UTR) | chr10 | 27400692 | 27400769 | − |
| SEQ ID NO. 701 | 3282254 | YME1L1 | Coding | chr10 | 27434387 | 27434415 | − |
| SEQ ID NO. 702 | 3284025 | KIF5B | Coding | chr10 | 32326449 | 32326532 | − |
| SEQ ID NO. 703 | 3284217 | ITGB1 | Coding | chr10 | 33218756 | 33218953 | − |
| SEQ ID NO. 704 | 3284603 | PARD3 | Non-coding (UTR) | chr10 | 34400052 | 34400096 | − |
| SEQ ID NO. 705 | 3284665 | PARD3 | Non-coding (UTR) | chr10 | 34601224 | 34601273 | − |
| SEQ ID NO. 706 | 3294341 | DNAJC9, MRPS16 | Non-coding (UTR) | chr10 | 75008616 | 75008693 | − |
| SEQ ID NO. 707 | 3294501 | PPP3CB | Non-coding (UTR) | chr10 | 75196574 | 75196612 | − |
| SEQ ID NO. 708 | 3298980 | GLUD1 | Non-coding (UTR) | chr10 | 88810786 | 88810903 | − |
| SEQ ID NO. 709 | 3301222 | PDLIM1 | Non-coding (UTR) | chr10 | 96997343 | 96997375 | − |
| SEQ ID NO. 710 | 3301703 | ZNF518A | Non-coding (Exonic | chr10 | 97919238 | 97919382 | − |
| | | | Antisense) | | | | |
| SEQ ID NO. 711 | 3301858 | TM9SF3 | Non-coding (UTR) | chr10 | 98277891 | 98277925 | − |
| SEQ ID NO. 712 | 3301863 | TM9SF3 | Non-coding (UTR) | chr10 | 98281306 | 98281546 | − |
| SEQ ID NO. 713 | 3302047 | LCOR | Non-coding (Exonic | chr10 | 98723611 | 98724126 | − |
| | | | Antisense) | | | | |
| SEQ ID NO. 714 | 3304013 | MGEA5 | Non-coding (UTR) | chr10 | 103544229 | 103544292 | − |
| SEQ ID NO. 715 | 3304626 | NT5C2 | Non-coding (UTR) | chr10 | 104847820 | 104847904 | − |
| SEQ ID NO. 716 | 3304652 | NT5C2 | Coding | chr10 | 104860992 | 104861082 | − |
| SEQ ID NO. 717 | 3307940 | ABLIM1 | Non-coding (UTR) | chr10 | 116190884 | 116190918 | − |
| SEQ ID NO. 718 | 3308532 | KIAA1598 | Coding | chr10 | 118700019 | 118700070 | − |
| SEQ ID NO. 719 | 3309267 | EIF3A | Coding | chr10 | 120816451 | 120816552 | − |
| SEQ ID NO. 720 | 3311304 | FAM53B | Non- | chr10 | 126370080 | 126370121 | − |
| | | | coding (Intronic) | | | | |
| SEQ ID NO. 721 | 3317557 | AC131971.3 | Non- | chr11 | 2965724 | 2965773 | + |
| | | | coding (ncTranscript) | | | | |
| SEQ ID NO. 722 | 3325714 | EIF3M | Coding | chr11 | 32623308 | 32623345 | + |
| SEQ ID NO. 723 | 3325963 | HIPK3 | Non-coding (UTR) | chr11 | 33378433 | 33378548 | + |
| SEQ ID NO. 724 | 3326246 | CAPRIN1 | Non-coding (UTR) | chr11 | 34122068 | 34122389 | + |
| SEQ ID NO. 725 | 3326465 | EHF | Non- | chr11 | 34644499 | 34644527 | + |
| | | | coding (Intronic) | | | | |
| SEQ ID NO. 726 | 3329941 | FNBP4 | Non-coding (Exonic | chr11 | 47738349 | 47738675 | + |
| | | | Antisense) | | | | |
| SEQ ID NO. 727 | 3331532 | CTNND1, RP11- | Non- | chr11 | 57530665 | 57530828 | + |
| | | 691N7.7, RP11- | coding (Intronic) | | | | |
| | | 691N7.6 | | | | | |
| SEQ ID NO. 728 | 3331573 | CTNND1 | Coding | chr11 | 57577586 | 57577659 | + |
| SEQ ID NO. 729 | 3331613 | C11orf31, RP11- | Non-coding (UTR) | chr11 | 57510340 | 57510456 | + |
| | | 691N7.6 | | | | | |
| SEQ ID NO. 730 | 3331614 | C11orf31 | Non-coding (UTR) | chr11 | 57510457 | 57510487 | + |
| SEQ ID NO. 731 | 3333687 | NXF1 | Non-coding (Exonic | chr11 | 62559649 | 62559673 | + |
| | | | Antisense) | | | | |
| SEQ ID NO. 732 | 3334162 | OTUB1 | Non-coding (UTR) | chr11 | 63765054 | 63765087 | + |
| SEQ ID NO. 733 | 3334515 | PRDX5 | Non-coding (UTR) | chr11 | 64089219 | 64089254 | + |
| SEQ ID NO. 734 | 3335173 | MALAT1 | Non- | chr11 | 65268541 | 65268772 | + |
| | | | coding (ncTranscript) | | | | |
| SEQ ID NO. 735 | 3335176 | MALAT1 | Non- | chr11 | 65269878 | 65270060 | + |
| | | | coding (ncTranscript) | | | | |
| SEQ ID NO. 736 | 3335178 | MALAT1 | Non- | chr11 | 65270566 | 65270941 | + |
| | | | coding (ncTranscript) | | | | |
| SEQ ID NO. 737 | 3335179 | MALAT1 | Non- | chr11 | 65271104 | 65271134 | + |
| | | | coding (ncTranscript) | | | | |
| SEQ ID NO. 738 | 3335181 | MALAT1 | Non- | chr11 | 65271308 | 65271537 | + |
| | | | coding (ncTranscript) | | | | |
| SEQ ID NO. 739 | 3335182 | MALAT1 | Non- | chr11 | 65271622 | 65271671 | + |
| | | | coding (ncTranscript) | | | | |
| SEQ ID NO. 740 | 3335187 | MALAT1 | Non- | chr11 | 65272277 | 65272473 | + |
| | | | coding (ncTranscript) | | | | |
| SEQ ID NO. 741 | 3335188 | MALAT1 | Non- | chr11 | 65272569 | 65272745 | + |
| | | | coding (ncTranscript) | | | | |
| SEQ ID NO. 742 | 3335189 | MALAT1 | Non- | chr11 | 65272758 | 65272932 | + |
| | | | coding (ncTranscript) | | | | |
| SEQ ID NO. 743 | 3335191 | MALAT1 | Non- | chr11 | 65273073 | 65273133 | + |
| | | | coding (ncTranscript) | | | | |
| SEQ ID NO. 744 | 3335231 | NEAT1 | Non- | chr11 | 65198362 | 65198453 | + |
| | | | coding (ncTranscript) | | | | |
| SEQ ID NO. 745 | 3337724 | PPP6R3 | Non-coding (UTR) | chr11 | 68380696 | 68382033 | + |
| SEQ ID NO. 746 | 3338618 | CTTN | Non-coding (UTR) | chr11 | 70282540 | 70282638 | + |
| SEQ ID NO. 747 | 3341466 | C11orf67 | Non-coding (UTR) | chr11 | 77583362 | 77583395 | + |
| SEQ ID NO. 748 | 3341504 | NDUFC2 | Non-coding (Exonic | chr11 | 77780928 | 77781047 | + |
| | | | Antisense) | | | | |
| SEQ ID NO. 749 | 3344853 | KIAA1731 | Non-coding (UTR) | chr11 | 93463465 | 93463495 | + |
| SEQ ID NO. 750 | 3344889 | C11orf54 | Non-coding (UTR) | chr11 | 93496161 | 93496200 | + |
| SEQ ID NO. 751 | 3345453 | ENDOD1 | Non-coding (UTR) | chr11 | 94865773 | 94865801 | + |
| SEQ ID NO. 752 | 3347691 | ATM | Coding | chr11 | 108138023 | 108138050 | + |
| SEQ ID NO. 753 | 3352533 | ARHGEF12 | Coding | chr11 | 120278450 | 120278520 | + |
| SEQ ID NO. 754 | 3352561 | ARHGEF12 | Coding | chr11 | 120316131 | 120316173 | + |
| SEQ ID NO. 755 | 3352585 | ARHGEF12 | Coding | chr11 | 120335995 | 120336070 | + |
| SEQ ID NO. 756 | 3352925 | SC5DL | Coding | chr11 | 121178187 | 121178220 | + |
| SEQ ID NO. 757 | 3354966 | NAPIL1P1 | Non- | chr11 | 125938163 | 125938203 | + |
| | | | coding (ncTranscript) | | | | |
| SEQ ID NO. 758 | 3356173 | APLP2 | Non-coding (UTR) | chr11 | 130013877 | 130014133 | + |
| SEQ ID NO. 759 | 3358364 | PDDC1 | Coding | chr11 | 767235 | 767266 | − |
| SEQ ID NO. 760 | 3359382 | KCNQ1 | Non-coding (Intronic | chr11 | 2717173 | 2717259 | − |
| | | | Antisense) | | | | |
| SEQ ID NO. 761 | 3362739 | EIF4G2 | Coding | chr11 | 10821117 | 10821149 | − |
| SEQ ID NO. 762 | 3362742 | EIF4G2 | Coding | chr11 | 10821676 | 10821713 | − |
| SEQ ID NO. 763 | 3362745 | EIF4G2 | Coding | chr11 | 10821991 | 10822194 | − |
| SEQ ID NO. 764 | 3362763 | EIF4G2 | Non- | chr11 | 10825209 | 10825432 | − |
| | | | coding (ncTranscript) | | | | |
| SEQ ID NO. 765 | 3362773 | EIF4G2 | Non- | chr11 | 10828449 | 10828622 | − |
| | | | coding (ncTranscript) | | | | |
| SEQ ID NO. 766 | 3362943 | ZBED5 | Non-coding (UTR) | chr11 | 10874366 | 10874401 | − |
| SEQ ID NO. 767 | 3363519 | TEAD1 | Non-coding (Exonic | chr11 | 12965256 | 12965362 | − |
| | | | Antisense) | | | | |
| SEQ ID NO. 768 | 3363981 | PSMA1 | Non-coding (UTR) | chr11 | 14526493 | 14526586 | − |
| SEQ ID NO. 769 | 3364729 | RPS13 | Coding | chr11 | 17096711 | 17096738 | − |
| SEQ ID NO. 770 | 3364770 | PIK3C2A | Coding | chr11 | 17112883 | 17112975 | − |
| SEQ ID NO. 771 | 3364800 | PIK3C2A | Non- | chr11 | 17150520 | 17150651 | − |
| | | | coding (Intronic) | | | | |
| SEQ ID NO. 772 | 3368920 | CAPRIN1 | Non-coding (Exonic | chr11 | 34121109 | 34121645 | − |
| | | | Antisense) | | | | |
| SEQ ID NO. 773 | 3371737 | CKAP5 | Coding | chr11 | 46780476 | 46780582 | − |
| SEQ ID NO. 774 | 3372461 | FNBP4 | Non-coding (UTR) | chr11 | 47738082 | 47738123 | − |
| SEQ ID NO. 775 | 3375309 | CYBASC3 | Non-coding (UTR) | chr11 | 61116287 | 61116314 | − |
| SEQ ID NO. 776 | 3375752 | AHNAK | Coding | chr11 | 62288763 | 62288870 | − |
| SEQ ID NO. 777 | 3375863 | MTA2 | Non-coding (UTR) | chr11 | 62360703 | 62360739 | − |
| SEQ ID NO. 778 | 3376090 | RP11- | Coding | chr11 | 62482992 | 62483073 | − |
| | | 831H9.16, HNRNP | | | | | |
| | | UL2 | | | | | |
| SEQ ID NO. 779 | 3376775 | STIP1 | Non-coding (Exonic | chr11 | 63971039 | 63971068 | − |
| | | | Antisense) | | | | |
| SEQ ID NO. 780 | 3377457 | ZNHIT2 | Non-coding (UTR) | chr11 | 64883876 | 64883909 | − |
| SEQ ID NO. 781 | 3377464 | FAU | Non-coding (UTR) | chr11 | 64888125 | 64888152 | − |
| SEQ ID NO. 782 | 3377621 | NEAT1 | Non-coding (Exonic | chr11 | 65197863 | 65198674 | − |
| | | | Antisense) | | | | |
| SEQ ID NO. 783 | 3377623 | NEAT1 | Non-coding (Exonic | chr11 | 65199459 | 65199866 | − |
| | | | Antisense) | | | | |
| SEQ ID NO. 784 | 3377625 | NEAT1 | Non-coding (Exonic | chr11 | 65200367 | 65201041 | − |
| | | | Antisense) | | | | |
| SEQ ID NO. 785 | 3377632 | NEAT1 | Non-coding (Exonic | chr11 | 65204836 | 65204978 | − |
| | | | Antisense) | | | | |
| SEQ ID NO. 786 | 3377633 | NEAT1 | Non-coding (Exonic | chr11 | 65205053 | 65205164 | − |
| | | | Antisense) | | | | |
| SEQ ID NO. 787 | 3377659 | MALAT1 | Non-coding (Exonic | chr11 | 65271852 | 65272483 | − |
| | | | Antisense) | | | | |
| SEQ ID NO. 788 | 3377660 | MALAT1 | Non-coding (Exonic | chr11 | 65272684 | 65273443 | − |
| | | | Antisense) | | | | |
| SEQ ID NO. 789 | 3379122 | RPL37P2 | Non- | chr11 | 67450210 | 67450359 | − |
| | | | coding (ncTranscript) | | | | |
| SEQ ID NO. 790 | 3379574 | PPP6R3 | Non-coding (Exonic | chr11 | 68382665 | 68382760 | − |
| | | | Antisense) | | | | |
| SEQ ID NO. 791 | 3382981 | RSF1 | Coding | chr11 | 77383104 | 77383245 | − |
| SEQ ID NO. 792 | 3382984 | RSF1 | Coding | chr11 | 77387892 | 77388043 | − |
| SEQ ID NO. 793 | 3383148 | NDUFC2 | Coding | chr11 | 77780919 | 77780950 | − |
| SEQ ID NO. 794 | 3383149 | NDUFC2 | Coding | chr11 | 77780951 | 77781016 | − |
| SEQ ID NO. 795 | 3384489 | CCDC90B | Coding | chr11 | 82984696 | 82984729 | − |
| SEQ ID NO. 796 | 3384495 | CCDC90B | Coding | chr11 | 82985729 | 82985759 | − |
| SEQ ID NO. 797 | 3385045 | SYTL2 | Non-coding (UTR) | chr11 | 85406077 | 85406206 | − |
| SEQ ID NO. 798 | 3385178 | chr11−: 85668255-85668284 | Non- | chr11 | 85668255 | 85668284 | − |
| | | | coding (Intergenic) | | | | |
| SEQ ID NO. 799 | 3386816 | TAF1D | Non-coding (UTR) | chr11 | 93463398 | 93463437 | − |
| SEQ ID NO. 800 | 3387176 | CWC15 | Non- | chr11 | 94696550 | 94696588 | − |
| | | | coding (ncTranscript) | | | | |
| SEQ ID NO. 801 | 3387436 | FAM76B | Non- | chr11 | 95512328 | 95512401 | − |
| | | | coding (ncTranscript) | | | | |
| SEQ ID NO. 802 | 3388633 | TMEM123 | Non-coding (UTR) | chr11 | 102268975 | 102269230 | − |
| SEQ ID NO. 803 | 3393789 | RP11-770J1.5 | Non- | chr11 | 118269809 | 118269884 | − |
| | | | coding (Intronic) | | | | |
| SEQ ID NO. 804 | 3394076 | RPS25 | Coding | chr11 | 118888186 | 118888210 | − |
| SEQ ID NO. 805 | 3394529 | PVRL1 | Non- | chr11 | 119561120 | 119561157 | − |
| | | | coding (Intronic) | | | | |
| SEQ ID NO. 806 | 3395420 | HSPA8 | Non-coding (UTR) | chr11 | 122928373 | 122928441 | − |
| SEQ ID NO. 807 | 3395427 | HSPA8 | Coding | chr11 | 122929366 | 122929425 | − |
| SEQ ID NO. 808 | 3400152 | WNK1 | Non- | chr12 | 997620 | 997790 | + |
| | | | coding (Intronic) | | | | |
| SEQ ID NO. 809 | 3402624 | NCAPD2 | Non-coding (UTR) | chr12 | 6640984 | 6641092 | + |
| SEQ ID NO. 810 | 3402667 | CHD4 | Non-coding (Exonic | chr12 | 6679340 | 6679405 | + |
| | | | Antisense) | | | | |
| SEQ ID NO. 811 | 3402720 | COPS7A | Non-coding (UTR) | chr12 | 6840982 | 6841012 | + |
| SEQ ID NO. 812 | 3406062 | ATF7IP | Coding | chr12 | 14578178 | 14578407 | + |
| SEQ ID NO. 813 | 3407177 | PLEKHA5 | Non- | chr12 | 19462808 | 19462891 | + |
| | | | coding (Intronic) | | | | |
| SEQ ID NO. 814 | 3407275 | AEBP2 | Non-coding (UTR) | chr12 | 19673066 | 19673136 | + |
| SEQ ID NO. 815 | 3409292 | PPFIBP1 | Coding | chr12 | 27845759 | 27845805 | + |
| SEQ ID NO. 816 | 3412651 | ANO6 | Coding | chr12 | 45822918 | 45822963 | + |
| SEQ ID NO. 817 | 3414226 | TMBIM6 | Non-coding (UTR) | chr12 | 50158602 | 50158686 | + |
| SEQ ID NO. 818 | 3416068 | PCBP2 | Coding | chr12 | 53862564 | 53862605 | + |
| SEQ ID NO. 819 | 3416501 | HNRNPA1 | Non-coding (UTR) | chr12 | 54678679 | 54679003 | + |
| SEQ ID NO. 820 | 3417160 | CDK2 | Non-coding (UTR) | chr12 | 56366537 | 56366564 | + |
| SEQ ID NO. 821 | 3417666 | ATP5B | Non-coding (Exonic | chr12 | 57032010 | 57032118 | + |
| | | | Antisense) | | | | |
| SEQ ID NO. 822 | 3418209 | MARS | Non-coding (UTR) | chr12 | 57910381 | 57910412 | + |
| SEQ ID NO. 823 | 3419262 | MON2 | Coding | chr12 | 62895382 | 62895464 | + |
| SEQ ID NO. 824 | 3419622 | RP11-415112.6 | Non- | chr12 | 64216729 | 64216755 | + |
| | | | coding (ncTranscript) | | | | |
| SEQ ID NO. 825 | 3421154 | RAP1B | Non-coding (UTR) | chr12 | 69054240 | 69054272 | + |
| SEQ ID NO. 826 | 3421223 | NUP107 | Non-coding (UTR) | chr12 | 69136358 | 69136416 | + |
| SEQ ID NO. 827 | 3421350 | MDM2 | Non-coding (UTR) | chr12 | 69237819 | 69237941 | + |
| SEQ ID NO. 828 | 3421492 | CPSF6 | Non-coding (UTR) | chr12 | 69663759 | 69663896 | + |
| SEQ ID NO. 829 | 3421652 | CCT2 | Coding | chr12 | 69986764 | 69986823 | + |
| SEQ ID NO. 830 | 3421668 | CCT2 | Non-coding (UTR) | chr12 | 69995199 | 69995277 | + |
| SEQ ID NO. 831 | 3427811 | TMPO | Non-coding (UTR) | chr12 | 98944024 | 98944069 | + |
| SEQ ID NO. 832 | 3427812 | TMPO | Non-coding (UTR) | chr12 | 98944093 | 98944124 | + |
| SEQ ID NO. 833 | 3428711 | CHPT1 | Non-coding (UTR) | chr12 | 102122724 | 102122790 | + |
| SEQ ID NO. 834 | 3428712 | CHPT1 | Non-coding (UTR) | chr12 | 102122801 | 102122832 | + |
| SEQ ID NO. 835 | 3429794 | KIAA1033 | Coding | chr12 | 105538106 | 105538233 | + |
| SEQ ID NO. 836 | 3430575 | PWP1 | Non-coding (UTR) | chr12 | 108106058 | 108106208 | + |
| SEQ ID NO. 837 | 3431352 | TCHP | Non-coding (UTR) | chr12 | 110355835 | 110355871 | + |
| SEQ ID NO. 838 | 3431686 | PPP1CC | Non-coding (Exonic | chr12 | 111157751 | 111158527 | + |
| | | | Antisense) | | | | |
| SEQ ID NO. 839 | 3432171 | MAPKAPK5 | Non-coding (UTR) | chr12 | 112331155 | 112331227 | + |
| SEQ ID NO. 840 | 3432352 | PTPN11 | Coding | chr12 | 112915472 | 112915526 | + |
| SEQ ID NO. 841 | 3435860 | TMED2 | Coding | chr12 | 124074920 | 124074969 | + |
| SEQ ID NO. 842 | 3435864 | TMED2 | Non-coding (UTR) | chr12 | 124081749 | 124082527 | + |
| SEQ ID NO. 843 | 3436017 | TCTN2 | Non-coding (UTR) | chr12 | 124192827 | 124192906 | + |
| SEQ ID NO. 844 | 3436264 | ZNF664 | Non-coding (UTR) | chr12 | 124499930 | 124499958 | + |
| SEQ ID NO. 845 | 3438044 | RAN | Non-coding (UTR) | chr12 | 131360699 | 131360737 | + |
| SEQ ID NO. 846 | 3445545 | PLBD1 | Non-coding (UTR) | chr12 | 14656642 | 14656700 | − |
| SEQ ID NO. 847 | 3445674 | WBPI1 | Non-coding (UTR) | chr12 | 14939813 | 14939844 | − |
| SEQ ID NO. 848 | 3445750 | MGP | Coding | chr12 | 15035935 | 15035980 | − |
| SEQ ID NO. 849 | 3446488 | RP11-841C19.6 | Non- | chr12 | 19610131 | 19610161 | − |
| | | | coding (ncTranscript) | | | | |
| SEQ ID NO. 850 | 3446869 | LDHB | Non-coding (UTR) | chr12 | 21788309 | 21788372 | − |
| SEQ ID NO. 851 | 3446885 | LDHB | Coding | chr12 | 21796891 | 21796927 | − |
| SEQ ID NO. 852 | 3452256 | SLC38A1 | Coding | chr12 | 46594896 | 46594950 | − |
| SEQ ID NO. 853 | 3452262 | SLC38A1 | Coding | chr12 | 46598093 | 46598153 | − |
| SEQ ID NO. 854 | 3452324 | SLC38A2 | Non-coding (UTR) | chr12 | 46752021 | 46752132 | − |
| SEQ ID NO. 855 | 3452349 | SLC38A2 | Non- | chr12 | 46759827 | 46760034 | − |
| | | | coding (Intronic) | | | | |
| SEQ ID NO. 856 | 3454729 | DAZAP2 | Non-coding (Exonic | chr12 | 51637043 | 51637499 | − |
| | | | Antisense) | | | | |
| SEQ ID NO. 857 | 3456599 | SMUG1 | Non-coding (UTR) | chr12 | 54575266 | 54575301 | − |
| SEQ ID NO. 858 | 3457550 | ANKRD52 | Non-coding (UTR) | chr12 | 56631625 | 56631704 | − |
| SEQ ID NO. 859 | 3458494 | R3HDM2 | Coding | chr12 | 57690242 | 57690331 | − |
| SEQ ID NO. 860 | 3461302 | RAP1B | Non-coding (Exonic | chr12 | 69054237 | 69054275 | − |
| | | | Antisense) | | | | |
| SEQ ID NO. 861 | 3461431 | CPSF6 | Non-coding (Intronic | chr12 | 69660063 | 69660148 | − |
| | | | Antisense) | | | | |
| SEQ ID NO. 862 | 3462710 | KRR1 | Coding | chr12 | 75895562 | 75895592 | − |
| SEQ ID NO. 863 | 3462846 | NAP1L1 | Non-coding (UTR) | chr12 | 76440105 | 76440134 | − |
| SEQ ID NO. 864 | 3462861 | NAP1L1 | Coding | chr12 | 76443241 | 76443271 | − |
| SEQ ID NO. 865 | 3462867 | NAP1L1 | Coding | chr12 | 76447550 | 76447578 | − |
| SEQ ID NO. 866 | 3462868 | NAP1L1 | Coding | chr12 | 76447613 | 76447637 | − |
| SEQ ID NO. 867 | 3462884 | NAP1L1 | Coding | chr12 | 76462692 | 76462753 | − |
| SEQ ID NO. 868 | 3462988 | OSBPL8 | Coding | chr12 | 76796488 | 76796545 | − |
| SEQ ID NO. 869 | 3463596 | PPP1R12A | Coding | chr12 | 80189429 | 80189478 | − |
| SEQ ID NO. 870 | 3463603 | PPP1R12A | Coding | chr12 | 80192274 | 80192363 | − |
| SEQ ID NO. 871 | 3464009 | CCDC59 | Non-coding (UTR) | chr12 | 82746826 | 82746850 | − |
| SEQ ID NO. 872 | 3465251 | LUM | Non-coding (UTR) | chr12 | 91497423 | 91497674 | − |
| SEQ ID NO. 873 | 3465252 | LUM | Non-coding (UTR) | chr12 | 91497768 | 91497898 | − |
| SEQ ID NO. 874 | 3465278 | DCN | Non-coding (UTR) | chr12 | 91539375 | 91539470 | − |
| SEQ ID NO. 875 | 3466861 | CDK17 | Coding | chr12 | 96692676 | 96692724 | − |
| SEQ ID NO. 876 | 3467642 | UHRF1BP1L | Non-coding (UTR) | chr12 | 100430873 | 100430903 | − |
| SEQ ID NO. 877 | 3468015 | ARL1 | Coding | chr12 | 101789418 | 101789448 | − |
| SEQ ID NO. 878 | 3469369 | APPL2 | Coding | chr12 | 105601936 | 105601968 | − |
| SEQ ID NO. 879 | 3471177 | ATP2A2 | Non-coding (Exonic | chr12 | 110760804 | 110760877 | − |
| | | | Antisense) | | | | |
| SEQ ID NO. 880 | 3471377 | PPP1CC | Coding | chr12 | 111157748 | 111158008 | − |
| SEQ ID NO. 881 | 3471379 | PPP1CC | Non-coding (UTR) | chr12 | 111158304 | 111158805 | − |
| SEQ ID NO. 882 | 3472092 | RPL6 | Coding | chr12 | 112843756 | 112843788 | − |
| SEQ ID NO. 883 | 3473482 | FBXO21 | Non-coding (UTR) | chr12 | 117581623 | 117581716 | − |
| SEQ ID NO. 884 | 3474494 | COX6A1 | Non-coding (Exonic | chr12 | 120878384 | 120878408 | − |
| | | | Antisense) | | | | |
| SEQ ID NO. 885 | 3474937 | ANAPC5 | Non-coding (UTR) | chr12 | 121746187 | 121746219 | − |
| SEQ ID NO. 886 | 3475623 | CLIP1 | Coding | chr12 | 122812874 | 122812960 | − |
| SEQ ID NO. 887 | 3475737 | RSRC2 | Coding | chr12 | 123003467 | 123003552 | − |
| SEQ ID NO. 888 | 3476294 | RP11-338K17.8 | Non- | chr12 | 124192881 | 124192911 | − |
| | | | coding (ncTranscript) | | | | |
| SEQ ID NO. 889 | 3480098 | PSPC1 | Non-coding (Intronic | chr13 | 20288513 | 20288582 | + |
| | | | Antisense) | | | | |
| SEQ ID NO. 890 | 3480100 | PSPC1 | Non-coding (Intronic | chr13 | 20289439 | 20289587 | + |
| | | | Antisense) | | | | |
| SEQ ID NO. 891 | 3480173 | ZMYM2 | Coding | chr13 | 20625705 | 20625738 | + |
| SEQ ID NO. 892 | 3480201 | ZMYM2 | Coding | chr13 | 20660015 | 20660105 | + |
| SEQ ID NO. 893 | 3480203 | ZMYM2 | Non-coding (UTR) | chr13 | 20660684 | 20660800 | + |
| SEQ ID NO. 894 | 3480685 | MRP63 | Non-coding (UTR) | chr13 | 21751366 | 21751479 | + |
| SEQ ID NO. 895 | 3480856 | RP11-149B7.3 | Non- | chr13 | 21949514 | 21949608 | + |
| | | | coding (Intronic) | | | | |
| SEQ ID NO. 896 | 3481480 | MIPEP | Non-coding (Exonic | chr13 | 24304376 | 24304486 | + |
| | | | Antisense) | | | | |
| SEQ ID NO. 897 | 3482124 | PABPC3 | Coding | chr13 | 25671061 | 25671086 | + |
| SEQ ID NO. 898 | 3482915 | GTF3A | Non-coding (UTR) | chr13 | 28009740 | 28009770 | + |
| SEQ ID NO. 899 | 3482916 | GTF3A | Non-coding (UTR) | chr13 | 28009783 | 28009821 | + |
| SEQ ID NO. 900 | 3483215 | EEF1A1P3 | Non- | chr13 | 28790004 | 28790032 | + |
| | | | coding (ncTranscript) | | | | |
| SEQ ID NO. 901 | 3484743 | N4BP2L2 | Non-coding (Exonic | chr13 | 33091777 | 33091983 | + |
| | | | Antisense) | | | | |
| SEQ ID NO. 902 | 3484750 | N4BP2L2 | Non-coding (Intronic | chr13 | 33100404 | 33100549 | + |
| | | | Antisense) | | | | |
| SEQ ID NO. 903 | 3484756 | N4BP2L2 | Non-coding (Intronic | chr13 | 33108310 | 33108639 | + |
| | | | Antisense) | | | | |
| SEQ ID NO. 904 | 3484762 | N4BP2L2 | Non-coding (Exonic | chr13 | 33110810 | 33111128 | + |
| | | | Antisense) | | | | |
| SEQ ID NO. 905 | 3485891 | EXOSC8 | Non-coding (UTR) | chr13 | 37583482 | 37583513 | + |
| SEQ ID NO. 906 | 3489010 | ITM2B | Non-coding (UTR) | chr13 | 48835362 | 48835441 | + |
| SEQ ID NO. 907 | 3489011 | ITM2B | Non-coding (UTR) | chr13 | 48835461 | 48835490 | + |
| SEQ ID NO. 908 | 3494193 | LMO7 | Coding | chr13 | 76378428 | 76378461 | + |
| SEQ ID NO. 909 | 3497656 | MBNL2 | Coding | chr13 | 98043613 | 98043670 | + |
| SEQ ID NO. 910 | 3497658 | MBNL2 | Non-coding (UTR) | chr13 | 98046231 | 98046350 | + |
| SEQ ID NO. 911 | 3497823 | IPO5 | Coding | chr13 | 98658384 | 98658526 | + |
| SEQ ID NO. 912 | 3498048 | FARP1 | Non-coding (UTR) | chr13 | 99101993 | 99102022 | + |
| SEQ ID NO. 913 | 3498445 | UBAC2 | Non-coding (UTR) | chr13 | 100038537 | 100038628 | + |
| SEQ ID NO. 914 | 3498523 | TM9SF2 | Coding | chr13 | 100181729 | 100181759 | + |
| SEQ ID NO. 915 | 3498537 | TM9SF2 | Coding | chr13 | 100193828 | 100193859 | + |
| SEQ ID NO. 916 | 3498977 | PCCA | Non-coding (UTR) | chr13 | 101182613 | 101182666 | + |
| SEQ ID NO. 917 | 3499545 | TPP2 | Coding | chr13 | 103328647 | 103328727 | + |
| SEQ ID NO. 918 | 3500408 | ARGLU1 | Non-coding (Exonic | chr13 | 107210889 | 107211226 | + |
| | | | Antisense) | | | | |
| SEQ ID NO. 919 | 3501216 | COL4A1 | Non-coding (Exonic | chr13 | 110801366 | 110802521 | + |
| | | | Antisense) | | | | |
| SEQ ID NO. 920 | 3501217 | COL4A1 | Non-coding (Exonic | chr13 | 110802557 | 110802622 | + |
| | | | Antisense) | | | | |
| SEQ ID NO. 921 | 3501555 | ANKRD10, RP11- | Non-coding (Intronic | chr13 | 111550126 | 111550263 | + |
| | | 120J20.2 | Antisense) | | | | |
| SEQ ID NO. 922 | 3503211 | CDC16 | Non-coding (UTR) | chr13 | 115038049 | 115038141 | + |
| SEQ ID NO. 923 | 3505453 | MIPEP | Non-coding (UTR) | chr13 | 24304376 | 24304406 | − |
| SEQ ID NO. 924 | 3506941 | GTF3A | Non-coding (Exonic | chr13 | 28009271 | 28009319 | − |
| | | | Antisense) | | | | |
| SEQ ID NO. 925 | 3508731 | N4BP2L2 | Non-coding (UTR) | chr13 | 33091139 | 33091739 | − |
| SEQ ID NO. 926 | 3508732 | N4BP2L2 | Non-coding (UTR) | chr13 | 33091797 | 33091904 | − |
| SEQ ID NO. 927 | 3508761 | N4BP2L2 | Non-coding (UTR) | chr13 | 33112755 | 33112784 | − |
| SEQ ID NO. 928 | 3509912 | FAM48A | Non-coding (UTR) | chr13 | 37583472 | 37583574 | − |
| SEQ ID NO. 929 | 3509913 | FAM48A | Non-coding (UTR) | chr13 | 37583672 | 37583718 | − |
| SEQ ID NO. 930 | 3510070 | POSTN | Coding | chr13 | 38137470 | 38137505 | − |
| SEQ ID NO. 931 | 3510382 | C13orf23 | Coding | chr13 | 39591637 | 39591681 | − |
| SEQ ID NO. 932 | 3512558 | TPT1 | Coding | chr13 | 45914196 | 45914249 | − |
| SEQ ID NO. 933 | 3513710 | FNDC3A | Non-coding (Intronic | chr13 | 49743307 | 49743495 | − |
| | | | Antisense) | | | | |
| SEQ ID NO. 934 | 3517677 | KLF5 | Non-coding (Intronic | chr13 | 73639022 | 73639125 | − |
| | | | Antisense) | | | | |
| SEQ ID NO. 935 | 3517683 | KLF5 | Non-coding (Exonic | chr13 | 73651458 | 73651592 | − |
| | | | Antisense) | | | | |
| SEQ ID NO. 936 | 3518501 | MYCBP2 | Non-coding (UTR) | chr13 | 77618823 | 77618901 | − |
| SEQ ID NO. 937 | 3519136 | RBM26 | Coding | chr13 | 79908511 | 79908559 | − |
| SEQ ID NO. 938 | 3521962 | MBNL2 | Non-coding (Exonic | chr13 | 98046114 | 98046289 | − |
| | | | Antisense) | | | | |
| SEQ ID NO. 939 | 3524627 | ARGLU1 | Coding | chr13 | 107209416 | 107209478 | − |
| SEQ ID NO. 940 | 3524630 | ARGLU1 | Non- | chr13 | 107209980 | 107210978 | − |
| | | | coding (ncTranscript) | | | | |
| SEQ ID NO. 941 | 3524631 | ARGLU1 | Non- | chr13 | 107211047 | 107211103 | − |
| | | | coding (Intronic) | | | | |
| SEQ ID NO. 942 | 3525314 | COL4A1 | Non-coding (UTR) | chr13 | 110801323 | 110801391 | − |
| SEQ ID NO. 943 | 3525317 | COL4A1 | Non-coding (UTR) | chr13 | 110802009 | 110802049 | − |
| SEQ ID NO. 944 | 3525320 | COL4A1 | Non-coding (UTR) | chr13 | 110802659 | 110802703 | − |
| SEQ ID NO. 945 | 3525547 | CARS2 | Non-coding (UTR) | chr13 | 111293770 | 111293801 | − |
| SEQ ID NO. 946 | 3525702 | ANKRD10 | Coding | chr13 | 111546459 | 111546509 | − |
| SEQ ID NO. 947 | 3525704 | ANKRD10 | Non- | chr13 | 111546562 | 111546615 | − |
| | | | coding (Intronic) | | | | |
| SEQ ID NO. 948 | 3525710 | ANKRD10, RP11- | Non- | chr13 | 111550458 | 111550798 | − |
| | | 120J20.2 | coding (Intronic) | | | | |
| SEQ ID NO. 949 | 3526383 | PCID2 | Non-coding (UTR) | chr13 | 113832145 | 113832173 | − |
| SEQ ID NO. 950 | 3527530 | PNP | Non-coding (UTR) | chr14 | 20945198 | 20945234 | + |
| SEQ ID NO. 951 | 3528208 | chr14+: 22219841-22219898 | Non- | chr14 | 22219841 | 22219898 | + |
| | | | coding (Intergenic) | | | | |
| SEQ ID NO. 952 | 3529094 | PABPN1 | Coding | chr14 | 23793174 | 23793252 | + |
| SEQ ID NO. 953 | 3531094 | SCFD1 | Non-coding (UTR) | chr14 | 31204908 | 31204938 | + |
| SEQ ID NO. 954 | 3531491 | ARHGAP5 | Non-coding (UTR) | chr14 | 32559824 | 32559857 | + |
| SEQ ID NO. 955 | 3533452 | PNN | Coding | chr14 | 39649881 | 39650067 | + |
| SEQ ID NO. 956 | 3534950 | KLHDC2 | Non-coding (UTR) | chr14 | 50249686 | 50249814 | + |
| SEQ ID NO. 957 | 3535917 | PSMC6 | Non-coding (UTR) | chr14 | 53194359 | 53194390 | + |
| SEQ ID NO. 958 | 3536669 | MAPK11P1L | Non- | chr14 | 55521391 | 55521561 | + |
| | | | coding (Intronic) | | | | |
| SEQ ID NO. 959 | 3536745 | LGALS3 | Non-coding (UTR) | chr14 | 55612028 | 55612094 | + |
| SEQ ID NO. 960 | 3536931 | KTN1 | Coding | chr14 | 56079043 | 56079161 | + |
| SEQ ID NO. 961 | 3536934 | KTN1 | Coding | chr14 | 56084718 | 56084852 | + |
| SEQ ID NO. 962 | 3536938 | KTN1 | Coding | chr14 | 56094637 | 56094745 | + |
| SEQ ID NO. 963 | 3536943 | KTN1 | Coding | chr14 | 56103204 | 56103241 | + |
| SEQ ID NO. 964 | 3536948 | KTN1 | Coding | chr14 | 56107121 | 56107158 | + |
| SEQ ID NO. 965 | 3536949 | KTN1 | Coding | chr14 | 56107654 | 56107685 | + |
| SEQ ID NO. 966 | 3536951 | KTN1 | Coding | chr14 | 56108443 | 56108473 | + |
| SEQ ID NO. 967 | 3536992 | KTN1 | Coding | chr14 | 56138319 | 56138348 | + |
| SEQ ID NO. 968 | 3536994 | KTN1 | Coding | chr14 | 56138552 | 56138592 | + |
| SEQ ID NO. 969 | 3537787 | PSMA3 | Non- | chr14 | 58736530 | 58737088 | + |
| | | | coding (Intronic) | | | | |
| SEQ ID NO. 970 | 3537795 | PSMA3 | Non-coding (UTR) | chr14 | 58738645 | 58738676 | + |
| SEQ ID NO. 971 | 3537796 | PSMA3 | Non-coding (UTR) | chr14 | 58738678 | 58738711 | + |
| SEQ ID NO. 972 | 3539104 | HIF1A | Non-coding (UTR) | chr14 | 62213835 | 62214927 | + |
| SEQ ID NO. 973 | 3539877 | SYNE2 | Coding | chr14 | 64608087 | 64608217 | + |
| SEQ ID NO. 974 | 3543929 | ZNF410 | Non-coding (UTR) | chr14 | 74398925 | 74398960 | + |
| SEQ ID NO. 975 | 3544473 | TMED10 | Non-coding (Exonic | chr14 | 75600819 | 75601043 | + |
| | | | Antisense) | | | | |
| SEQ ID NO. 976 | 3544890 | C14orf179 | Non-coding (UTR) | chr14 | 76549980 | 76550008 | + |
| SEQ ID NO. 977 | 3545534 | C14orf156 | Coding | chr14 | 78183857 | 78183893 | + |
| SEQ ID NO. 978 | 3549043 | GOLGA5 | Coding | chr14 | 93273192 | 93273296 | + |
| SEQ ID NO. 979 | 3550451 | PAPOLA | Non-coding (UTR) | chr14 | 97031464 | 97031570 | + |
| SEQ ID NO. 980 | 3550453 | PAPOLA | Non-coding (UTR) | chr14 | 97032230 | 97033320 | + |
| SEQ ID NO. 981 | 3550455 | PAPOLA | Non-coding (UTR) | chr14 | 97033359 | 97033402 | + |
| SEQ ID NO. 982 | 3552937 | DYNC1H1 | Coding | chr14 | 102500662 | 102500721 | + |
| SEQ ID NO. 983 | 3552938 | DYNC1H1 | Coding | chr14 | 102500737 | 102500788 | + |
| SEQ ID NO. 984 | 3556198 | HNRNPC | Non- | chr14 | 21711637 | 21711681 | − |
| | | | coding (Intronic) | | | | |
| SEQ ID NO. 985 | 3556224 | HNRNPC | Non- | chr14 | 21715371 | 21715455 | − |
| | | | coding (Intronic) | | | | |
| SEQ ID NO. 986 | 3556278 | HNRNPC | Non- | chr14 | 21724188 | 21724274 | − |
| | | | coding (Intronic) | | | | |
| SEQ ID NO. 987 | 3556420 | METTL3 | Non-coding (UTR) | chr14 | 21966312 | 21966385 | − |
| SEQ ID NO. 988 | 3558292 | SDR39U1 | Non-coding (UTR) | chr14 | 24909005 | 24909035 | − |
| SEQ ID NO. 989 | 3559519 | STRN3 | Coding | chr14 | 31388179 | 31388221 | − |
| SEQ ID NO. 990 | 3559556 | STRN3 | Non- | chr14 | 31494313 | 31494364 | − |
| | | | coding (Intronic) | | | | |
| SEQ ID NO. 991 | 3562436 | chr14−: 43624685-43624788 | Non- | chr14 | 43624685 | 43624788 | − |
| | | | coding (Intergenic) | | | | |
| SEQ ID NO. 992 | 3563349 | KLHDC2 | Non-coding (Exonic | chr14 | 50249079 | 50249817 | − |
| | | | Antisense) | | | | |
| SEQ ID NO. 993 | 3563970 | MAP4K5 | Coding | chr14 | 50930810 | 50930847 | − |
| SEQ ID NO. 994 | 3566596 | C14orf37 | Non- | chr14 | 58732098 | 58732170 | − |
| | | | coding (Intronic) | | | | |
| SEQ ID NO. 995 | 3566603 | C14orf37 | Non- | chr14 | 58738232 | 58738695 | − |
| | | | coding (Intronic) | | | | |
| SEQ ID NO. 996 | 3566648 | C14orf37 | Non- | chr14 | 58745642 | 58745917 | − |
| | | | coding (Intronic) | | | | |
| SEQ ID NO. 997 | 3566657 | TIMM9 | Non-coding (UTR) | chr14 | 58875404 | 58875448 | − |
| SEQ ID NO. 998 | 3566993 | JKAMP | Non-coding (Exonic | chr14 | 59971339 | 59971383 | − |
| | | | Antisense) | | | | |
| SEQ ID NO. 999 | 3569346 | PIGH | Non-coding (UTR) | chr14 | 68056048 | 68056249 | − |
| SEQ ID NO. 1000 | 3569757 | ZFP36L1 | Non-coding (UTR) | chr14 | 69254887 | 69255185 | − |
| SEQ ID NO. 1001 | 3569759 | ZFP36L1 | Non-coding (UTR) | chr14 | 69255920 | 69256161 | − |
| SEQ ID NO. 1002 | 3569819 | ACTN1 | Non-coding (UTR) | chr14 | 69341254 | 69341307 | − |
| SEQ ID NO. 1003 | 3570456 | COX16 | Non-coding (UTR) | chr14 | 70792813 | 70792939 | − |
| SEQ ID NO. 1004 | 3573155 | SPTLC2 | Non-coding (UTR) | chr14 | 77978379 | 77978415 | − |
| SEQ ID NO. 1005 | 3576551 | SMEK1 | Non-coding (UTR) | chr14 | 91924009 | 91924055 | − |
| SEQ ID NO. 1006 | 3576567 | SMEK1 | Coding | chr14 | 91929149 | 91929176 | − |
| SEQ ID NO. 1007 | 3576712 | TC2N | Non-coding (UTR) | chr14 | 92248983 | 92249066 | − |
| SEQ ID NO. 1008 | 3576730 | TC2N | Coding | chr14 | 92278706 | 92278866 | − |
| SEQ ID NO. 1009 | 3576939 | NDUFB1 | Non-coding (UTR) | chr14 | 92582506 | 92582534 | − |
| SEQ ID NO. 1010 | 3576940 | NDUFB1 | Coding | chr14 | 92582542 | 92582573 | − |
| SEQ ID NO. 1011 | 3577874 | DICER1 | Non-coding (UTR) | chr14 | 95556680 | 95556725 | − |
| SEQ ID NO. 1012 | 3577898 | DICER1 | Coding | chr14 | 95573945 | 95574043 | − |
| SEQ ID NO. 1013 | 3577911 | DICER1 | Coding | chr14 | 95583981 | 95584063 | − |
| SEQ ID NO. 1014 | 3578431 | PAPOLA | Non-coding (Exonic | chr14 | 97032931 | 97033315 | − |
| | | | Antisense) | | | | |
| SEQ ID NO. 1015 | 3580188 | HSP90AA1 | Coding | chr14 | 102549575 | 102549608 | − |
| SEQ ID NO. 1016 | 3580204 | HSP90AA1 | Coding | chr14 | 102552344 | 102552461 | − |
| SEQ ID NO. 1017 | 3580587 | CDC42BPB | Coding | chr14 | 103478471 | 103478503 | − |
| SEQ ID NO. 1018 | 3580953 | C14orf2 | Non-coding (UTR) | chr14 | 104378874 | 104378902 | − |
| SEQ ID NO. 1019 | 3589660 | SRP14 | Non-coding (Exonic | chr15 | 40327993 | 40328048 | + |
| | | | Antisense) | | | | |
| SEQ ID NO. 1020 | 3590159 | ZFYVE19 | Non-coding (UTR) | chr15 | 41106724 | 41106757 | + |
| SEQ ID NO. 1021 | 3590352 | AC012652.1 | Coding | chr15 | 41554969 | 41555013 | + |
| SEQ ID NO. 1022 | 3591851 | CASC4 | Coding | chr15 | 44615169 | 44615209 | + |
| SEQ ID NO. 1023 | 3591858 | CASC4 | Coding | chr15 | 44624186 | 44624269 | + |
| SEQ ID NO. 1024 | 3591985 | EIF3J | Non-coding (UTR) | chr15 | 44853865 | 44853895 | + |
| SEQ ID NO. 1025 | 3592046 | B2M | Non-coding (UTR) | chr15 | 45010076 | 45010140 | + |
| SEQ ID NO. 1026 | 3592047 | B2M | Non-coding (UTR) | chr15 | 45010149 | 45010306 | + |
| SEQ ID NO. 1027 | 3592395 | SPATA5L1 | Non-coding (UTR) | chr15 | 45713540 | 45713570 | + |
| SEQ ID NO. 1028 | 3593171 | DUT | Non-coding (UTR) | chr15 | 48634495 | 48634524 | + |
| SEQ ID NO. 1029 | 3594101 | TMOD3 | Non-coding (UTR) | chr15 | 52203518 | 52204078 | + |
| SEQ ID NO. 1030 | 3595811 | ADAM10 | Non-coding (Exonic | chr15 | 58936101 | 58936173 | + |
| | | | Antisense) | | | | |
| SEQ ID NO. 1031 | 3595872 | FAM63B | Coding | chr15 | 59146705 | 59146747 | + |
| SEQ ID NO. 1032 | 3596235 | RPS3AP6 | Non- | chr15 | 60061048 | 60061073 | + |
| | | | coding (ncTranscript) | | | | |
| SEQ ID NO. 1033 | 3597406 | TPM1 | Coding | chr15 | 63363335 | 63363371 | + |
| SEQ ID NO. 1034 | 3597468 | RPS27L | Non-coding (Exonic | chr15 | 63448685 | 63448718 | + |
| | | | Antisense) | | | | |
| SEQ ID NO. 1035 | 3597671 | USP3 | Non- | chr15 | 63863224 | 63863330 | + |
| | | | coding (Intronic) | | | | |
| SEQ ID NO. 1036 | 3598717 | RPL4 | Non-coding (Exonic | chr15 | 66792495 | 66792526 | + |
| | | | Antisense) | | | | |
| SEQ ID NO. 1037 | 3599449 | FEM1B | Non-coding (UTR) | chr15 | 68588065 | 68588154 | + |
| SEQ ID NO. 1038 | 3602509 | UBE2Q2 | Coding | chr15 | 76165783 | 76165852 | + |
| SEQ ID NO. 1039 | 3605249 | BTBD1 | Non-coding (Exonic | chr15 | 83685253 | 83685989 | + |
| | | | Antisense) | | | | |
| SEQ ID NO. 1040 | 3606117 | PDE8A | Coding | chr15 | 85660888 | 85661024 | + |
| SEQ ID NO. 1041 | 3606409 | AKAP13 | Coding | chr15 | 86228040 | 86228071 | + |
| SEQ ID NO. 1042 | 3608201 | IQGAP1 | Non-coding (UTR) | chr15 | 91043485 | 91044457 | + |
| SEQ ID NO. 1043 | 3609166 | chr15+: 93441269-93441296 | Non- | chr15 | 93441269 | 93441296 | + |
| | | | coding (Intergenic) | | | | |
| SEQ ID NO. 1044 | 3615597 | TJP1 | Coding | chr15 | 29993813 | 29993887 | − |
| SEQ ID NO. 1045 | 3615604 | TJP1 | Coding | chr15 | 30000769 | 30001056 | − |
| SEQ ID NO. 1046 | 3615659 | TJP1 | Coding | chr15 | 30064323 | 30064365 | − |
| SEQ ID NO. 1047 | 3615989 | MTMR10 | Non-coding (UTR) | chr15 | 31231241 | 31231375 | − |
| SEQ ID NO. 1048 | 3621014 | RP11-473C18.1 | Non- | chr15 | 43408169 | 43408193 | − |
| | | | coding (ncTranscript) | | | | |
| SEQ ID NO. 1049 | 3623329 | SECISBP2L | Non-coding (UTR) | chr15 | 49280903 | 49280978 | − |
| SEQ ID NO. 1050 | 3624199 | DMXL2 | Coding | chr15 | 51828398 | 51828822 | − |
| SEQ ID NO. 1051 | 3624533 | MYO5C | Coding | chr15 | 52511971 | 52512014 | − |
| SEQ ID NO. 1052 | 3624702 | ARPP19 | Non-coding (UTR) | chr15 | 52842989 | 52843020 | − |
| SEQ ID NO. 1053 | 3625565 | NEDD4 | Coding | chr15 | 56144633 | 56144664 | − |
| SEQ ID NO. 1054 | 3626570 | ADAM10 | Coding | chr15 | 58889656 | 58889693 | − |
| SEQ ID NO. 1055 | 3626585 | ADAM10 | Coding | chr15 | 58913702 | 58913746 | − |
| SEQ ID NO. 1056 | 3626730 | SLTM | Coding | chr15 | 59186138 | 59186165 | − |
| SEQ ID NO. 1057 | 3627045 | GTF2A2 | Non-coding (UTR) | chr15 | 59931155 | 59931320 | − |
| SEQ ID NO. 1058 | 3628011 | VPS13C | Coding | chr15 | 62223290 | 62223365 | − |
| SEQ ID NO. 1059 | 3628052 | VPS13C | Coding | chr15 | 62277071 | 62277198 | − |
| SEQ ID NO. 1060 | 3628057 | VPS13C | Coding | chr15 | 62299573 | 62299653 | − |
| SEQ ID NO. 1061 | 3628471 | RPS27L | Non-coding (UTR) | chr15 | 63446088 | 63446161 | − |
| SEQ ID NO. 1062 | 3628924 | FAM96A | Non-coding (UTR) | chr15 | 64364822 | 64365114 | − |
| SEQ ID NO. 1063 | 3630191 | RPL4 | Coding | chr15 | 66793304 | 66793342 | − |
| SEQ ID NO. 1064 | 3631414 | UACA | Coding | chr15 | 70963386 | 70963413 | − |
| SEQ ID NO. 1065 | 3632170 | HEXA | Non-coding (UTR) | chr15 | 72636242 | 72636274 | − |
| SEQ ID NO. 1066 | 3633110 | ULK3 | Non-coding (UTR) | chr15 | 75128476 | 75128503 | − |
| SEQ ID NO. 1067 | 3633223 | COX5A | Non-coding (UTR) | chr15 | 75212727 | 75212760 | − |
| SEQ ID NO. 1068 | 3633226 | COX5A | Coding | chr15 | 75219110 | 75219150 | − |
| SEQ ID NO. 1069 | 3634074 | TSPAN3 | Non-coding (UTR) | chr15 | 77338406 | 77338505 | − |
| SEQ ID NO. 1070 | 3643958 | IFT140 | Non-coding (Intronic | chr16 | 1605465 | 1605543 | + |
| | | | Antisense) | | | | |
| SEQ ID NO. 1071 | 3645267 | SRRM2 | Coding | chr16 | 2809141 | 2809173 | + |
| SEQ ID NO. 1072 | 3645314 | SRRM2 | Coding | chr16 | 2821320 | 2821352 | + |
| SEQ ID NO. 1073 | 3645591 | AC108134.1 | Non-coding (UTR) | chr16 | 3082688 | 3082721 | + |
| SEQ ID NO. 1074 | 3647535 | PMM2 | Non-coding (UTR) | chr16 | 8943097 | 8943149 | + |
| SEQ ID NO. 1075 | 3648373 | GSPT1 | Non-coding (Exonic | chr16 | 11965914 | 11966088 | + |
| | | | Antisense) | | | | |
| SEQ ID NO. 1076 | 3651472 | THUMPD1 | Non-coding (Exonic | chr16 | 20745035 | 20745197 | + |
| | | | Antisense) | | | | |
| SEQ ID NO. 1077 | 3653349 | RBBP6 | Coding | chr16 | 24572962 | 24573036 | + |
| SEQ ID NO. 1078 | 3653673 | LCMT1 | Non-coding (UTR) | chr16 | 25189503 | 25189534 | + |
| SEQ ID NO. 1079 | 3655101 | ATP2A1 | Coding | chr16 | 28915742 | 28915771 | + |
| SEQ ID NO. 1080 | 3658610 | chr16+: 34329397-34329426 | Non- | chr16 | 34329397 | 34329426 | + |
| | | | coding (Intergenic) | | | | |
| SEQ ID NO. 1081 | 3659201 | PHKB | Coding | chr16 | 47674954 | 47674997 | + |
| SEQ ID NO. 1082 | 3659319 | LONP2 | Coding | chr16 | 48295347 | 48295482 | + |
| SEQ ID NO. 1083 | 3660917 | CHD9 | Coding | chr16 | 53256556 | 53256653 | + |
| SEQ ID NO. 1084 | 3660927 | CHD9 | Coding | chr16 | 53269097 | 53269212 | + |
| SEQ ID NO. 1085 | 3665635 | CTCF | Non-coding (UTR) | chr16 | 67673041 | 67673073 | + |
| SEQ ID NO. 1086 | 3666111 | NFATC3 | Non-coding (UTR) | chr16 | 68262372 | 68262449 | + |
| SEQ ID NO. 1087 | 3666869 | NFAT5 | Non-coding (UTR) | chr16 | 69738402 | 69738519 | + |
| SEQ ID NO. 1088 | 3668992 | CFDP1 | Non-coding (Exonic | chr16 | 75445798 | 75445831 | + |
| | | | Antisense) | | | | |
| SEQ ID NO. 1089 | 3671113 | HSD17B2 | Non-coding (UTR) | chr16 | 82132049 | 82132091 | + |
| SEQ ID NO. 1090 | 3677863 | CREBBP | Coding | chr16 | 3832776 | 3832921 | − |
| SEQ ID NO. 1091 | 3679601 | USP7 | Coding | chr16 | 9002201 | 9002297 | − |
| SEQ ID NO. 1092 | 3683054 | SMG1 | Non-coding (UTR) | chr16 | 18816225 | 18816252 | − |
| SEQ ID NO. 1093 | 3685080 | USP31 | Coding | chr16 | 23113641 | 23113757 | − |
| SEQ ID NO. 1094 | 3687262 | CDIPT | Non-coding (UTR) | chr16 | 29869699 | 29869726 | − |
| SEQ ID NO. 1095 | 3687792 | DCTPP1 | Non-coding (UTR) | chr16 | 30435055 | 30435119 | − |
| SEQ ID NO. 1096 | 3690089 | DNAJA2 | Non-coding (UTR) | chr16 | 46990519 | 46990548 | − |
| SEQ ID NO. 1097 | 3692857 | AMFR | Non-coding (UTR) | chr16 | 56395400 | 56395436 | − |
| SEQ ID NO. 1098 | 3693546 | CSNK2A2 | Non- | chr16 | 58197918 | 58197957 | − |
| | | | coding (Intronic) | | | | |
| SEQ ID NO. 1099 | 3693711 | CNOT1 | Coding | chr16 | 58580290 | 58580397 | − |
| SEQ ID NO. 1100 | 3693737 | CNOT1 | Coding | chr16 | 58612675 | 58612766 | − |
| SEQ ID NO. 1101 | 3693746 | CNOT1 | Coding | chr16 | 58621684 | 58621741 | − |
| SEQ ID NO. 1102 | 3696670 | NFAT5 | Non-coding (Exonic | chr16 | 69735142 | 69735208 | − |
| | | | Antisense) | | | | |
| SEQ ID NO. 1103 | 3697925 | KIAA0174 | Non-coding (Intronic | chr16 | 71956876 | 71957154 | − |
| | | | Antisense) | | | | |
| SEQ ID NO. 1104 | 3699559 | CFDP1 | Coding | chr16 | 75445743 | 75445831 | − |
| SEQ ID NO. 1105 | 3701588 | chr16−: 81422942-81422986 | Non- | chr16 | 81422942 | 81422986 | − |
| | | | coding (Intergenic) | | | | |
| SEQ ID NO. 1106 | 3705531 | GLOD4 | Non-coding (Exonic | chr17 | 662604 | 662642 | + |
| | | | Antisense) | | | | |
| SEQ ID NO. 1107 | 3707211 | PSMB6 | Non-coding (UTR) | chr17 | 4701718 | 4701745 | + |
| SEQ ID NO. 1108 | 3708006 | TXNDC17 | Non-coding (UTR) | chr17 | 6546346 | 6546406 | + |
| SEQ ID NO. 1109 | 3709275 | CHD3 | Coding | chr17 | 7801317 | 7801402 | + |
| SEQ ID NO. 1110 | 3710735 | MAP2K4 | Non-coding (UTR) | chr17 | 12044587 | 12044730 | + |
| SEQ ID NO. 1111 | 3714102 | ALDH3A2 | Non-coding (UTR) | chr17 | 19578985 | 19579015 | + |
| SEQ ID NO. 1112 | 3715142 | WSB1 | Non-coding (UTR) | chr17 | 25639650 | 25640711 | + |
| SEQ ID NO. 1113 | 3715434 | NLK | Non-coding (UTR) | chr17 | 26523346 | 26523402 | + |
| SEQ ID NO. 1114 | 3716021 | NUFIP2 | Non-coding (Exonic | chr17 | 27584395 | 27585068 | + |
| | | | Antisense) | | | | |
| SEQ ID NO. 1115 | 3716674 | SUZ12P | Non- | chr17 | 29061929 | 29061961 | + |
| | | | coding (ncTranscript) | | | | |
| SEQ ID NO. 1116 | 3716757 | CRLF3 | Non-coding (Exonic | chr17 | 29109181 | 29110227 | + |
| | | | Antisense) | | | | |
| SEQ ID NO. 1117 | 3717647 | ZNF207 | Coding | chr17 | 30688498 | 30688534 | + |
| SEQ ID NO. 1118 | 3717681 | ZNF207 | Non-coding (UTR) | chr17 | 30703222 | 30704388 | + |
| SEQ ID NO. 1119 | 3717684 | ZNF207 | Non-coding (UTR) | chr17 | 30707067 | 30707332 | + |
| SEQ ID NO. 1120 | 3718837 | TAF15 | Non-coding (UTR) | chr17 | 34174149 | 34174181 | + |
| SEQ ID NO. 1121 | 3719123 | ZNHIT3 | Non-coding (UTR) | chr17 | 34851541 | 34851568 | + |
| SEQ ID NO. 1122 | 3720778 | MSL1 | Non-coding (UTR) | chr17 | 38292930 | 38292996 | + |
| SEQ ID NO. 1123 | 3720990 | TOP2A | Non-coding (Exonic | chr17 | 38551732 | 38551789 | + |
| | | | Antisense) | | | | |
| SEQ ID NO. 1124 | 3722990 | GPATCH8 | Non-coding (Exonic | chr17 | 42544382 | 42544480 | + |
| | | | Antisense) | | | | |
| SEQ ID NO. 1125 | 3723305 | NMT1 | Non-coding (UTR) | chr17 | 43183284 | 43183313 | + |
| SEQ ID NO. 1126 | 3726811 | LUC7L3 | Non-coding (UTR) | chr17 | 48829837 | 48829910 | + |
| SEQ ID NO. 1127 | 3728316 | chr17+: 55758561-55758676 | Non- | chr17 | 55758561 | 55758676 | + |
| | | | coding (Intergenic) | | | | |
| SEQ ID NO. 1128 | 3729194 | CLTC | Coding | chr17 | 57743467 | 57743600 | + |
| SEQ ID NO. 1129 | 3729227 | CLTC | Non-coding (UTR) | chr17 | 57771220 | 57771523 | + |
| SEQ ID NO. 1130 | 3732475 | BPTF | Coding | chr17 | 65905772 | 65905867 | + |
| SEQ ID NO. 1131 | 3732477 | BPTF | Coding | chr17 | 65906998 | 65907253 | + |
| SEQ ID NO. 1132 | 3732479 | BPTF | Coding | chr17 | 65908073 | 65908103 | + |
| SEQ ID NO. 1133 | 3732480 | BPTF | Coding | chr17 | 65908105 | 65908152 | + |
| SEQ ID NO. 1134 | 3734754 | NUP85 | Non-coding (UTR) | chr17 | 73231775 | 73231813 | + |
| SEQ ID NO. 1135 | 3734863 | KIAA0195 | Non-coding (UTR) | chr17 | 73496070 | 73496097 | + |
| SEQ ID NO. 1136 | 3737304 | RNF213 | Coding | chr17 | 78286838 | 78286959 | + |
| SEQ ID NO. 1137 | 3737985 | ACTG1 | Non-coding (Exonic | chr17 | 79477609 | 79477714 | + |
| | | | Antisense) | | | | |
| SEQ ID NO. 1138 | 3738300 | ANAPC11 | Non-coding (UTR) | chr17 | 79858058 | 79858126 | + |
| SEQ ID NO. 1139 | 3739125 | FN3KRP | Non-coding (UTR) | chr17 | 80685795 | 80685853 | + |
| SEQ ID NO. 1140 | 3739523 | RPH3AL | Non-coding (UTR) | chr17 | 62308 | 62338 | − |
| SEQ ID NO. 1141 | 3740129 | YWHAE | Non-coding (UTR) | chr17 | 1247864 | 1247932 | − |
| SEQ ID NO. 1142 | 3740130 | YWHAE | Non-coding (UTR) | chr17 | 1247937 | 1247962 | − |
| SEQ ID NO. 1143 | 3744423 | RPL26 | Coding | chr17 | 8280885 | 8280918 | − |
| SEQ ID NO. 1144 | 3746953 | NCOR1 | Coding | chr17 | 16041478 | 16041507 | − |
| SEQ ID NO. 1145 | 3746967 | NCOR1 | Coding | chr17 | 16055260 | 16055312 | − |
| SEQ ID NO. 1146 | 3747223 | UBB, AC093484.6 | Non-coding (Exonic | chr17 | 16285909 | 16285939 | − |
| | | | Antisense) | | | | |
| SEQ ID NO. 1147 | 3748953 | ALDH3A2 | Non-coding (Exonic | chr17 | 19578961 | 19579027 | − |
| | | | Antisense) | | | | |
| SEQ ID NO. 1148 | 3750786 | SPAG5 | Non-coding (UTR) | chr17 | 26904610 | 26904639 | − |
| SEQ ID NO. 1149 | 3752711 | MYO1D | Non-coding (UTR) | chr17 | 30819629 | 30819657 | − |
| SEQ ID NO. 1150 | 3754530 | ACACA | Coding | chr17 | 35538144 | 35538267 | − |
| SEQ ID NO. 1151 | 3754741 | DDX52 | Non-coding (UTR) | chr17 | 35974190 | 35974305 | − |
| SEQ ID NO. 1152 | 3756204 | TOP2A | Coding | chr17 | 38552572 | 38552698 | − |
| SEQ ID NO. 1153 | 3759590 | PLCD3 | Non-coding (UTR) | chr17 | 43189041 | 43189118 | − |
| SEQ ID NO. 1154 | 3760197 | KIAA1267 | Non- | chr17 | 44229099 | 44229138 | − |
| | | | coding (Intronic) | | | | |
| SEQ ID NO. 1155 | 3761752 | ZNF652 | Non- | chr17 | 47371242 | 47371658 | − |
| | | | coding (Intronic) | | | | |
| SEQ ID NO. 1156 | 3762575 | SPAG9 | Coding | chr17 | 49098531 | 49098605 | − |
| SEQ ID NO. 1157 | 3764121 | SRSF1 | Non-coding (UTR) | chr17 | 56080881 | 56081053 | − |
| SEQ ID NO. 1158 | 3764125 | SRSF1 | Non-coding (UTR) | chr17 | 56082334 | 56082402 | − |
| SEQ ID NO. 1159 | 3764127 | SRSF1 | Non-coding (UTR) | chr17 | 56082659 | 56082766 | − |
| SEQ ID NO. 1160 | 3764884 | PTRH2 | Non-coding (UTR) | chr17 | 57774692 | 57774781 | − |
| SEQ ID NO. 1161 | 3764921 | TMEM49 | Non-coding (Intronic | chr17 | 57916786 | 57917067 | − |
| | | | Antisense) | | | | |
| SEQ ID NO. 1162 | 3765738 | MED13 | Coding | chr17 | 60072562 | 60072692 | − |
| SEQ ID NO. 1163 | 3765761 | MED13 | Coding | chr17 | 60112841 | 60112917 | − |
| SEQ ID NO. 1164 | 3765767 | MED13 | Coding | chr17 | 60129924 | 60130040 | − |
| SEQ ID NO. 1165 | 3766896 | DDX5 | Coding | chr17 | 62496067 | 62496341 | − |
| SEQ ID NO. 1166 | 3768039 | HELZ | Coding | chr17 | 65110458 | 65110513 | − |
| SEQ ID NO. 1167 | 3768121 | PSMD12 | Coding | chr17 | 65343474 | 65343574 | − |
| SEQ ID NO. 1168 | 3768258 | BPTF | Non-coding (Exonic | chr17 | 65900527 | 65900955 | − |
| | | | Antisense) | | | | |
| SEQ ID NO. 1169 | 3769780 | SLC39A11 | Non-coding (UTR) | chr17 | 70642090 | 70642140 | − |
| SEQ ID NO. 1170 | 3770244 | RPL38 | Non-coding (Exonic | chr17 | 72205375 | 72205399 | − |
| | | | Antisense) | | | | |
| SEQ ID NO. 1171 | 3770564 | ATP5H | Non-coding (UTR) | chr17 | 73034985 | 73035011 | − |
| SEQ ID NO. 1172 | 3770565 | ATP5H | Coding | chr17 | 73035028 | 73035125 | − |
| SEQ ID NO. 1173 | 3770746 | AC011933.1, GRB2 | Non-coding (UTR) | chr17 | 73314175 | 73314221 | − |
| SEQ ID NO. 1174 | 3771039 | WBP2 | Non-coding (UTR) | chr17 | 73841911 | 73841967 | − |
| SEQ ID NO. 1175 | 3771094 | TRIM65 | Non-coding (UTR) | chr17 | 73885066 | 73885127 | − |
| SEQ ID NO. 1176 | 3771337 | EXOC7 | Non-coding (UTR) | chr17 | 74077120 | 74077149 | − |
| SEQ ID NO. 1177 | 3776189 | SMCHD1 | Coding | chr18 | 2694604 | 2694691 | + |
| SEQ ID NO. 1178 | 3776219 | SMCHD1 | Coding | chr18 | 2729285 | 2729399 | + |
| SEQ ID NO. 1179 | 3776236 | SMCHD1 | Coding | chr18 | 2760668 | 2760737 | + |
| SEQ ID NO. 1180 | 3776446 | MYL12A | Non-coding (UTR) | chr18 | 3256140 | 3256174 | + |
| SEQ ID NO. 1181 | 3776461 | MYL12B | Non-coding (UTR) | chr18 | 3277954 | 3277997 | + |
| SEQ ID NO. 1182 | 3778266 | ANKRD12 | Coding | chr18 | 9208709 | 9208785 | + |
| SEQ ID NO. 1183 | 3778629 | VAPA | Non-coding (UTR) | chr18 | 9954382 | 9954461 | + |
| SEQ ID NO. 1184 | 3778630 | VAPA | Non-coding (UTR) | chr18 | 9954599 | 9954786 | + |
| SEQ ID NO. 1185 | 3779850 | CEP192 | Coding | chr18 | 13042229 | 13042330 | + |
| SEQ ID NO. 1186 | 3781686 | RIOK3 | Coding | chr18 | 21043933 | 21043978 | + |
| SEQ ID NO. 1187 | 3781689 | RIOK3 | Coding | chr18 | 21044236 | 21044277 | + |
| SEQ ID NO. 1188 | 3783364 | DSC2 | Non-coding (Exonic | chr18 | 28645981 | 28646054 | + |
| | | | Antisense) | | | | |
| SEQ ID NO. 1189 | 3787934 | ACAA2 | Non-coding (Exonic | chr18 | 47309906 | 47309967 | + |
| | | | Antisense) | | | | |
| SEQ ID NO. 1190 | 3789817 | NARS | Non-coding (Exonic | chr18 | 55267984 | 55268652 | + |
| | | | Antisense) | | | | |
| SEQ ID NO. 1191 | 3795442 | HSBP1L1 | Non- | chr18 | 77726702 | 77726744 | + |
| | | | coding (Intronic) | | | | |
| SEQ ID NO. 1192 | 3796554 | MYL12A | Non-coding (Exonic | chr18 | 3253291 | 3253318 | − |
| | | | Antisense) | | | | |
| SEQ ID NO. 1193 | 3798470 | VAPA | Non-coding (Exonic | chr18 | 9954382 | 9954673 | − |
| | | | Antisense) | | | | |
| SEQ ID NO. 1194 | 3800623 | ROCK1 | Non-coding (UTR) | chr18 | 18529985 | 18530009 | − |
| SEQ ID NO. 1195 | 3804170 | RPRD1A | Coding | chr18 | 33605587 | 33605638 | − |
| SEQ ID NO. 1196 | 3804202 | SLC39A6 | Non-coding (UTR) | chr18 | 33689334 | 33689516 | − |
| SEQ ID NO. 1197 | 3806255 | ATP5A1 | Non-coding (UTR) | chr18 | 43664158 | 43664210 | − |
| SEQ ID NO. 1198 | 3806256 | ATP5A1 | Coding | chr18 | 43664262 | 43664314 | − |
| SEQ ID NO. 1199 | 3807476 | C18orf32 | Non-coding (UTR) | chr18 | 47008582 | 47008661 | − |
| SEQ ID NO. 1200 | 3807570 | ACAA2 | Non-coding (UTR) | chr18 | 47310009 | 47310100 | − |
| SEQ ID NO. 1201 | 3807629 | MYO5B | Coding | chr18 | 47405315 | 47405362 | − |
| SEQ ID NO. 1202 | 3808602 | MBD2 | Non-coding (UTR) | chr18 | 51681249 | 51681283 | − |
| SEQ ID NO. 1203 | 3809343 | TXNL1 | Non-coding (UTR) | chr18 | 54270079 | 54270117 | − |
| SEQ ID NO. 1204 | 3809673 | NARS | Non-coding (UTR) | chr18 | 55267919 | 55268047 | − |
| SEQ ID NO. 1205 | 3809834 | ATP8B1 | Coding | chr18 | 55319275 | 55319401 | − |
| SEQ ID NO. 1206 | 3813298 | CYB5A | Non-coding (UTR) | chr18 | 71920626 | 71920814 | − |
| SEQ ID NO. 1207 | 3813299 | CYB5A | Coding | chr18 | 71920819 | 71920861 | − |
| SEQ ID NO. 1208 | 3815669 | CIRBP | Non-coding (UTR) | chr19 | 1273068 | 1273101 | + |
| SEQ ID NO. 1209 | 3816402 | OAZ1 | Non-coding (UTR) | chr19 | 2273334 | 2273365 | + |
| SEQ ID NO. 1210 | 3820658 | SLC44A2 | Non-coding (UTR) | chr19 | 10755194 | 10755225 | + |
| SEQ ID NO. 1211 | 3820705 | ILF3 | Non-coding (UTR) | chr19 | 10796289 | 10796443 | + |
| SEQ ID NO. 1212 | 3820721 | ILF3 | Non-coding (UTR) | chr19 | 10803046 | 10803074 | + |
| SEQ ID NO. 1213 | 3820752 | QTRT1 | Non-coding (UTR) | chr19 | 10823990 | 10824021 | + |
| SEQ ID NO. 1214 | 3822055 | CALR | Coding | chr19 | 13050910 | 13050952 | + |
| SEQ ID NO. 1215 | 3825428 | UPF1 | Non-coding (UTR) | chr19 | 18978996 | 18979030 | + |
| SEQ ID NO. 1216 | 3829039 | PDCD5 | Coding | chr19 | 33078171 | 33078199 | + |
| SEQ ID NO. 1217 | 3829810 | UBA2 | Non-coding (UTR) | chr19 | 34960742 | 34960773 | + |
| SEQ ID NO. 1218 | 3831128 | WDR62 | Non-coding (UTR) | chr19 | 36595963 | 36596001 | + |
| SEQ ID NO. 1219 | 3831280 | ZNF146 | Non-coding (UTR) | chr19 | 36729581 | 36729635 | + |
| SEQ ID NO. 1220 | 3835412 | AC084219.1 | Non-coding (Intronic | chr19 | 44593725 | 44593755 | + |
| | | | Antisense) | | | | |
| SEQ ID NO. 1221 | 3837493 | GLTSCR2 | Coding | chr19 | 48259945 | 48259974 | + |
| SEQ ID NO. 1222 | 3838612 | RPS11 | Coding | chr19 | 50000777 | 50000849 | + |
| SEQ ID NO. 1223 | 3838790 | SCAF1 | Coding | chr19 | 50161065 | 50161099 | + |
| SEQ ID NO. 1224 | 3840344 | ZNF808 | Coding | chr19 | 53056690 | 53056733 | + |
| SEQ ID NO. 1225 | 3841271 | CNOT3 | Coding | chr19 | 54647403 | 54647485 | + |
| SEQ ID NO. 1226 | 3843668 | ZNF587 | Non-coding (UTR) | chr19 | 58375789 | 58375813 | + |
| SEQ ID NO. 1227 | 3845710 | AP3D1 | Non-coding (UTR) | chr19 | 2100996 | 2101030 | − |
| SEQ ID NO. 1228 | 3846115 | AES | Non-coding (UTR) | chr19 | 3052943 | 3053123 | − |
| SEQ ID NO. 1229 | 3846291 | C19orf29 | Coding | chr19 | 3610671 | 3610894 | − |
| SEQ ID NO. 1230 | 3846561 | EEF2 | Coding | chr19 | 3981383 | 3981450 | − |
| SEQ ID NO. 1231 | 3846784 | UBXN6 | Non-coding (UTR) | chr19 | 4445025 | 4445062 | − |
| SEQ ID NO. 1232 | 3847357 | LONP1 | Non-coding (UTR) | chr19 | 5691860 | 5691889 | − |
| SEQ ID NO. 1233 | 3849774 | chr19−: 9671051-9671089 | Non- | chr19 | 9671051 | 9671089 | − |
| | | | coding (Intergenic) | | | | |
| SEQ ID NO. 1234 | 3849776 | chr19−: 9671562-9671894 | Non- | chr19 | 9671562 | 9671894 | − |
| | | | coding (Intergenic) | | | | |
| SEQ ID NO. 1235 | 3850502 | AC011475.2 | Non-coding (UTR) | chr19 | 10762558 | 10762816 | − |
| SEQ ID NO. 1236 | 3851604 | DHPS | Non-coding (UTR) | chr19 | 12786536 | 12786648 | − |
| SEQ ID NO. 1237 | 3851902 | CALR | Non-coding (Exonic | chr19 | 13050901 | 13050963 | − |
| | | | Antisense) | | | | |
| SEQ ID NO. 1238 | 3854371 | MRPL34 | Non-coding (Exonic | chr19 | 17417528 | 17417592 | − |
| | | | Antisense) | | | | |
| SEQ ID NO. 1239 | 3855101 | C19orf50 | Non-coding (Exonic | chr19 | 18680064 | 18680145 | − |
| | | | Antisense) | | | | |
| SEQ ID NO. 1240 | 3856046 | chr19−: 19900909-19901230 | Non- | chr19 | 19900909 | 19901230 | − |
| | | | coding (Intergenic) | | | | |
| SEQ ID NO. 1241 | 3857120 | ZNF91 | Coding | chr19 | 23545201 | 23545314 | − |
| SEQ ID NO. 1242 | 3857884 | CTC-448F2.1 | Non- | chr19 | 30447703 | 30447769 | − |
| | | | coding (ncTranscript) | | | | |
| SEQ ID NO. 1243 | 3862168 | FBL | Non-coding (UTR) | chr19 | 40325111 | 40325177 | − |
| SEQ ID NO. 1244 | 3862473 | AKT2 | Non-coding (UTR) | chr19 | 40737024 | 40737088 | − |
| SEQ ID NO. 1245 | 3867099 | KDELR1 | Coding | chr19 | 48887509 | 48887616 | − |
| SEQ ID NO. 1246 | 3869659 | ZNF83 | Coding | chr19 | 53117048 | 53117096 | − |
| SEQ ID NO. 1247 | 3869971 | ZNF320 | Non- | chr19 | 53428386 | 53428415 | − |
| | | | coding (Intronic) | | | | |
| SEQ ID NO. 1248 | 3870593 | TSEN34 | Non-coding (Exonic | chr19 | 54697315 | 54697498 | − |
| | | | Antisense) | | | | |
| SEQ ID NO. 1249 | 3872197 | chr19−: 57806251-57806283 | Non- | chr19 | 57806251 | 57806283 | − |
| | | | coding (Intergenic) | | | | |
| SEQ ID NO. 1250 | 3872208 | chr19−: 57815574-57815765 | Non- | chr19 | 57815574 | 57815765 | − |
| | | | coding (Intergenic) | | | | |
| SEQ ID NO. 1251 | 3874114 | PTPRA | Coding | chr20 | 2967446 | 2967484 | + |
| SEQ ID NO. 1252 | 3874280 | ITPA | Non-coding (UTR) | chr20 | 3204423 | 3204497 | + |
| SEQ ID NO. 1253 | 3875278 | CRLS1 | Non-coding (UTR) | chr20 | 6018636 | 6018682 | + |
| SEQ ID NO. 1254 | 3878051 | DSTN | Non-coding (UTR) | chr20 | 17587973 | 17588000 | + |
| SEQ ID NO. 1255 | 3878053 | DSTN | Non-coding (UTR) | chr20 | 17588715 | 17588815 | + |
| SEQ ID NO. 1256 | 3879490 | XRN2 | Coding | chr20 | 21314576 | 21314607 | + |
| SEQ ID NO. 1257 | 3881731 | TM9SF4 | Non-coding (UTR) | chr20 | 30755023 | 30755057 | + |
| SEQ ID NO. 1258 | 3881914 | ASXL1 | Coding | chr20 | 31025076 | 31025127 | + |
| SEQ ID NO. 1259 | 3882710 | CHMP4B | Non-coding (UTR) | chr20 | 32442082 | 32442156 | + |
| SEQ ID NO. 1260 | 3882867 | ITCH | Coding | chr20 | 32996498 | 32996582 | + |
| SEQ ID NO. 1261 | 3883424 | ERGIC3 | Coding | chr20 | 34144991 | 34145020 | + |
| SEQ ID NO. 1262 | 3883533 | RBM39 | Non-coding (Exonic | chr20 | 34326915 | 34327120 | + |
| | | | Antisense) | | | | |
| SEQ ID NO. 1263 | 3883537 | RBM39 | Non-coding (Exonic | chr20 | 34328294 | 34328562 | + |
| | | | Antisense) | | | | |
| SEQ ID NO. 1264 | 3883987 | C20orf24 | Non-coding (UTR) | chr20 | 35240820 | 35240910 | + |
| SEQ ID NO. 1265 | 3884147 | RPN2 | Non-coding (UTR) | chr20 | 35869831 | 35869970 | + |
| SEQ ID NO. 1266 | 3884334 | CTNNBL1 | Non- | chr20 | 36345727 | 36345755 | + |
| | | | coding (Intronic) | | | | |
| SEQ ID NO. 1267 | 3884673 | RALGAPB | Coding | chr20 | 37153421 | 37153451 | + |
| SEQ ID NO. 1268 | 3884709 | RALGAPB | Non- | chr20 | 37196054 | 37196487 | + |
| | | | coding (Intronic) | | | | |
| SEQ ID NO. 1269 | 3885711 | LPIN3 | Non-coding (UTR) | chr20 | 39989186 | 39989218 | + |
| SEQ ID NO. 1270 | 3886656 | YWHAB | Non-coding (UTR) | chr20 | 43535667 | 43535846 | + |
| SEQ ID NO. 1271 | 3886907 | PIGT | Coding | chr20 | 44049169 | 44049270 | + |
| SEQ ID NO. 1272 | 3887661 | NCOA3 | Coding | chr20 | 46251022 | 46251063 | + |
| SEQ ID NO. 1273 | 3888081 | ARFGEF2 | Coding | chr20 | 47587735 | 47587844 | + |
| SEQ ID NO. 1274 | 3888182 | CSE1L | Non-coding (UTR) | chr20 | 47713056 | 47713100 | + |
| SEQ ID NO. 1275 | 3888253 | DDX27 | Non-coding (UTR) | chr20 | 47860499 | 47860571 | + |
| SEQ ID NO. 1276 | 3888289 | NCRNA00275 | Non- | chr20 | 47905708 | 47905750 | + |
| | | | coding (ncTranscript) | | | | |
| SEQ ID NO. 1277 | 3888494 | RNF114 | Coding | chr20 | 48568679 | 48568737 | + |
| SEQ ID NO. 1278 | 3888936 | DPM1 | Non-coding (Exonic | chr20 | 49551434 | 49551628 | + |
| | | | Antisense) | | | | |
| SEQ ID NO. 1279 | 3889140 | ATP9A | Non-coding (Exonic | chr20 | 50213352 | 50214559 | + |
| | | | Antisense) | | | | |
| SEQ ID NO. 1280 | 3889782 | PFDN4 | Non-coding (UTR) | chr20 | 52835863 | 52835936 | + |
| SEQ ID NO. 1281 | 3891221 | GNAS | Coding | chr20 | 57470705 | 57470737 | + |
| SEQ ID NO. 1282 | 3891257 | GNAS | Non-coding (UTR) | chr20 | 57485961 | 57486084 | + |
| SEQ ID NO. 1283 | 3892672 | RPS21 | Coding | chr20 | 60962934 | 60962970 | + |
| SEQ ID NO. 1284 | 3894074 | PCMTD2 | Non-coding (UTR) | chr20 | 62907409 | 62907536 | + |
| SEQ ID NO. 1285 | 3894508 | RP4- | Non-coding (Exonic | chr20 | 1147867 | 1148150 | − |
| | | 545L17.7, PSMF1 | Antisense) | | | | |
| SEQ ID NO. 1286 | 3894602 | FKBP1A | Non-coding (UTR) | chr20 | 1349656 | 1349683 | − |
| SEQ ID NO. 1287 | 3895000 | SNRPB | Non-coding (UTR) | chr20 | 2442340 | 2442373 | − |
| SEQ ID NO. 1288 | 3896202 | PCNA | Non-coding (UTR) | chr20 | 5095649 | 5095739 | − |
| SEQ ID NO. 1289 | 3898306 | SEL1L2 | Non- | chr20 | 13908674 | 13908801 | − |
| | | | coding (ncTranscript) | | | | |
| SEQ ID NO. 1290 | 3901666 | C20orf3 | Non-coding (UTR) | chr20 | 24943603 | 24943644 | − |
| SEQ ID NO. 1291 | 3902984 | CDK5RAP1 | Non-coding (UTR) | chr20 | 31946673 | 31946723 | − |
| SEQ ID NO. 1292 | 3903290 | EIF2S2 | Non-coding (UTR) | chr20 | 32677392 | 32677416 | − |
| SEQ ID NO. 1293 | 3904028 | chr20−: 34146096-34146124 | Non- | chr20 | 34146096 | 34146124 | − |
| | | | coding (Intergenic) | | | | |
| SEQ ID NO. 1294 | 3904029 | FER1L4, AL389875.1 | Non-coding (UTR) | chr20 | 34146544 | 34146635 | − |
| SEQ ID NO. 1295 | 3904255 | RBM39 | Coding | chr20 | 34317239 | 34317283 | − |
| SEQ ID NO. 1296 | 3904257 | RBM39 | Coding | chr20 | 34317385 | 34317431 | − |
| SEQ ID NO. 1297 | 3904276 | RBM39 | Non- | chr20 | 34327102 | 34327246 | − |
| | | | coding (ncTranscript) | | | | |
| SEQ ID NO. 1298 | 3904281 | RBM39 | Non- | chr20 | 34328529 | 34328635 | − |
| | | | coding (ncTranscript) | | | | |
| SEQ ID NO. 1299 | 3906196 | CHD6 | Coding | chr20 | 40083276 | 40083334 | − |
| SEQ ID NO. 1300 | 3907788 | SLC35C2 | Non-coding (UTR) | chr20 | 44978376 | 44978406 | − |
| SEQ ID NO. 1301 | 3908789 | STAU1 | Non-coding (UTR) | chr20 | 47729881 | 47730036 | − |
| SEQ ID NO. 1302 | 3909376 | ADNP | Non- | chr20 | 49531572 | 49531914 | − |
| | | | coding (Intronic) | | | | |
| SEQ ID NO. 1303 | 3911474 | VAPB | Non-coding (Exonic | chr20 | 57020464 | 57020567 | − |
| | | | Antisense) | | | | |
| SEQ ID NO. 1304 | 3911564 | STX16, RP11- | Non-coding (Exonic | chr20 | 57252189 | 57252339 | − |
| | | 261P9.5 | Antisense) | | | | |
| SEQ ID NO. 1305 | 3911706 | GNAS | Non-coding (Exonic | chr20 | 57470704 | 57470737 | − |
| | | | Antisense) | | | | |
| SEQ ID NO. 1306 | 3911738 | GNAS | Non-coding (Exonic | chr20 | 57485755 | 57485993 | − |
| | | | Antisense) | | | | |
| SEQ ID NO. 1307 | 3911801 | ATP5E | Coding | chr20 | 57605376 | 57605455 | − |
| SEQ ID NO. 1308 | 3911815 | SLMO2 | Non-coding (UTR) | chr20 | 57608287 | 57608462 | − |
| SEQ ID NO. 1309 | 3913564 | DIDO1 | Coding | chr20 | 61536907 | 61536953 | − |
| SEQ ID NO. 1310 | 3915125 | USP25 | Coding | chr21 | 17202860 | 17202896 | + |
| SEQ ID NO. 1311 | 3917896 | SOD1 | Non-coding (UTR) | chr21 | 33041107 | 33041153 | + |
| SEQ ID NO. 1312 | 3918586 | IFNAR1 | Coding | chr21 | 34715912 | 34715982 | + |
| SEQ ID NO. 1313 | 3918651 | IFNGR2 | Non- | chr21 | 34804818 | 34804850 | + |
| | | | coding (Intronic) | | | | |
| SEQ ID NO. 1314 | 3918723 | SON | Coding | chr21 | 34927046 | 34927291 | + |
| SEQ ID NO. 1315 | 3918734 | SON | Coding | chr21 | 34931543 | 34931653 | + |
| SEQ ID NO. 1316 | 3919024 | MRPS6 | Non-coding (UTR) | chr21 | 35515288 | 35515318 | + |
| SEQ ID NO. 1317 | 3920401 | TTC3 | Coding | chr21 | 38466361 | 38466389 | + |
| SEQ ID NO. 1318 | 3920456 | TTC3 | Coding | chr21 | 38555078 | 38555116 | + |
| SEQ ID NO. 1319 | 3920487 | TTC3 | Non-coding (UTR) | chr21 | 38574158 | 38575271 | + |
| SEQ ID NO. 1320 | 3922023 | FAM3B | Coding | chr21 | 42717624 | 42717654 | + |
| SEQ ID NO. 1321 | 3922993 | PKNOX1 | Coding | chr21 | 44445014 | 44445052 | + |
| SEQ ID NO. 1322 | 3924758 | DIP2A | Non-coding (UTR) | chr21 | 47989552 | 47989586 | + |
| SEQ ID NO. 1323 | 3925641 | NRIP1 | Non-coding (UTR) | chr21 | 16333709 | 16333966 | − |
| SEQ ID NO. 1324 | 3926090 | CXADR | Non-coding (Exonic | chr21 | 18940282 | 18940430 | − |
| | | | Antisense) | | | | |
| SEQ ID NO. 1325 | 3927180 | ATP5J | Non-coding (UTR) | chr21 | 27096864 | 27096895 | − |
| SEQ ID NO. 1326 | 3927228 | APP | Non-coding (UTR) | chr21 | 27253137 | 27253203 | − |
| SEQ ID NO. 1327 | 3927229 | APP | Non-coding (UTR) | chr21 | 27253256 | 27253928 | − |
| SEQ ID NO. 1328 | 3928670 | TIAM1 | Coding | chr21 | 32490760 | 32490873 | − |
| SEQ ID NO. 1329 | 3929943 | ATP5O, AP000304.12 | Coding | chr21 | 35279673 | 35279756 | − |
| SEQ ID NO. 1330 | 3934699 | PTTG1IP | Coding | chr21 | 46271504 | 46271534 | − |
| SEQ ID NO. 1331 | 3935246 | LSS, AP001468.2 | Non-coding (UTR) | chr21 | 47609049 | 47609184 | − |
| SEQ ID NO. 1332 | 3935301 | MCM3AP | Non-coding (UTR) | chr21 | 47655078 | 47655109 | − |
| SEQ ID NO. 1333 | 3936897 | MRPL40 | Non-coding (UTR) | chr22 | 19423556 | 19423583 | + |
| SEQ ID NO. 1334 | 3939407 | SMARCB1 | Non-coding (UTR) | chr22 | 24176665 | 24176692 | + |
| SEQ ID NO. 1335 | 3942668 | TUG1 | Non- | chr22 | 31372108 | 31372178 | + |
| | | | coding (ncTranscript) | | | | |
| SEQ ID NO. 1336 | 3945331 | DDX17 | Non-coding (Exonic | chr22 | 38881930 | 38881962 | + |
| | | | Antisense) | | | | |
| SEQ ID NO. 1337 | 3945370 | CBY1 | Non-coding (UTR) | chr22 | 39069809 | 39069840 | + |
| SEQ ID NO. 1338 | 3946374 | ADSL, RP5- | Coding | chr22 | 40762454 | 40762500 | + |
| | | 1042K10.13 | | | | | |
| SEQ ID NO. 1339 | 3946589 | XPNPEP3, RBX1 | Non-coding (UTR) | chr22 | 41368540 | 41368588 | + |
| SEQ ID NO. 1340 | 3946677 | EP300 | Coding | chr22 | 41566534 | 41566575 | + |
| SEQ ID NO. 1341 | 3953804 | PI4KA | Coding | chr22 | 21161651 | 21161735 | − |
| SEQ ID NO. 1342 | 3956591 | XBP1 | Non-coding (UTR) | chr22 | 29190562 | 29190620 | − |
| SEQ ID NO. 1343 | 3957262 | SF3A1 | Non-coding (UTR) | chr22 | 30730186 | 30730263 | − |
| SEQ ID NO. 1344 | 3959259 | RBFOX2 | Non- | chr22 | 36334548 | 36334674 | − |
| | | | coding (Intronic) | | | | |
| SEQ ID NO. 1345 | 3959453 | MYH9 | Non-coding (UTR) | chr22 | 36677371 | 36677403 | − |
| SEQ ID NO. 1346 | 3960634 | DDX17 | Non-coding (UTR) | chr22 | 38880599 | 38880760 | − |
| SEQ ID NO. 1347 | 3961482 | ADSL, RP5- | Non-coding (Exonic | chr22 | 40762473 | 40762565 | − |
| | | 1042K10.13 | Antisense) | | | | |
| SEQ ID NO. 1348 | 3962268 | NDUFA6 | Non-coding (UTR) | chr22 | 42482152 | 42482181 | − |
| SEQ ID NO. 1349 | 3962596 | AL049757.3 | Non- | chr22 | 43172653 | 43172678 | − |
| | | | coding (ncTranscript) | | | | |
| SEQ ID NO. 1350 | 3969510 | OFD1 | Coding | chrX | 13787185 | 13787222 | + |
| SEQ ID NO. 1351 | 3969511 | OFD1 | Non-coding (UTR) | chrX | 13787252 | 13787317 | + |
| SEQ ID NO. 1352 | 3970894 | PDHA1 | Coding | chrX | 19377802 | 19377939 | + |
| SEQ ID NO. 1353 | 3974757 | USP9X | Coding | chrX | 41043776 | 41043927 | + |
| SEQ ID NO. 1354 | 3974789 | USP9X | Coding | chrX | 41082579 | 41082644 | + |
| SEQ ID NO. 1355 | 3981154 | OGT | Coding | chrX | 70783211 | 70783337 | + |
| SEQ ID NO. 1356 | 3981752 | NCRNA00183 | Non- | chrX | 73219683 | 73219763 | + |
| | | | coding (ncTranscript) | | | | |
| SEQ ID NO. 1357 | 3981906 | MIR374AHG | Non-coding (Intronic | chrX | 73491128 | 73491377 | + |
| | | | Antisense) | | | | |
| SEQ ID NO. 1358 | 3982421 | COX7B | Non-coding (UTR) | chrX | 77160796 | 77160836 | + |
| SEQ ID NO. 1359 | 3985558 | NGFRAP1 | Non-coding (UTR) | chrX | 102632930 | 102632968 | + |
| SEQ ID NO. 1360 | 3985635 | TCEAL4 | Non-coding (UTR) | chrX | 102842274 | 102842422 | + |
| SEQ ID NO. 1361 | 3986857 | COL4A5 | Non-coding (UTR) | chrX | 107939988 | 107940724 | + |
| SEQ ID NO. 1362 | 3987532 | ALG13 | Non-coding (UTR) | chrX | 111003747 | 111003854 | + |
| SEQ ID NO. 1363 | 3988214 | RPI1-27G9.1 | Non- | chrX | 116097423 | 116097474 | + |
| | | | coding (ncTranscript) | | | | |
| SEQ ID NO. 1364 | 3989205 | MCTS1 | Non-coding (UTR) | chrX | 119746133 | 119746224 | + |
| SEQ ID NO. 1365 | 3989770 | STAG2 | Coding | chrX | 123196985 | 123197053 | + |
| SEQ ID NO. 1366 | 3991722 | HPRT1 | Non-coding (UTR) | chrX | 133634517 | 133634660 | + |
| SEQ ID NO. 1367 | 3993347 | CDR1 | Non-coding (Exonic | chrX | 139865845 | 139865911 | + |
| | | | Antisense) | | | | |
| SEQ ID NO. 1368 | 3993349 | CDR1 | Non-coding (Exonic | chrX | 139866456 | 139866504 | + |
| | | | Antisense) | | | | |
| SEQ ID NO. 1369 | 4004822 | DYNLT3 | Non-coding (UTR) | chrX | 37698478 | 37698663 | − |
| SEQ ID NO. 1370 | 4007593 | SLC35A2 | Non-coding (UTR) | chrX | 48760908 | 48760940 | − |
| SEQ ID NO. 1371 | 4009318 | HUWE1 | Non-coding (UTR) | chrX | 53559796 | 53559831 | − |
| SEQ ID NO. 1372 | 4009451 | HUWE1 | Coding | chrX | 53654361 | 53654464 | − |
| SEQ ID NO. 1373 | 4011604 | IGBP1 | Non-coding (Exonic | chrX | 69385996 | 69386136 | − |
| | | | Antisense) | | | | |
| SEQ ID NO. 1374 | 4011967 | OGT | Non-coding (Exonic | chrX | 70765919 | 70766276 | − |
| | | | Antisense) | | | | |
| SEQ ID NO. 1375 | 4012169 | RPS4X | Coding | chrX | 71495419 | 71495459 | − |
| SEQ ID NO. 1376 | 4012627 | MIR374AHG | Non- | chrX | 73473183 | 73473384 | − |
| | | | coding (Intronic) | | | | |
| SEQ ID NO. 1377 | 4012632 | MIR374AHG | Non- | chrX | 73477308 | 73477534 | − |
| | | | coding (Intronic) | | | | |
| SEQ ID NO. 1378 | 4012759 | chrX−: 73429697-73429730 | Non- | chrX | 73429697 | 73429730 | − |
| | | | coding (Intergenic) | | | | |
| SEQ ID NO. 1379 | 4013274 | ATRX | Coding | chrX | 76937974 | 76938071 | − |
| SEQ ID NO. 1380 | 4013282 | ATRX | Coding | chrX | 76939315 | 76939355 | − |
| SEQ ID NO. 1381 | 4015535 | HNRNPA1P27 | Non- | chrX | 100143214 | 100143253 | − |
| | | | coding (ncTranscript) | | | | |
| SEQ ID NO. 1382 | 4016549 | TCEAL4 | Non-coding (Exonic | chrX | 102842274 | 102842394 | − |
| | | | Antisense) | | | | |
| SEQ ID NO. 1383 | 4016573 | MORF4L2 | Non-coding (UTR) | chrX | 102930491 | 102930528 | − |
| SEQ ID NO. 1384 | 4019367 | PGRMC1 | Non-coding (Exonic | chrX | 118377548 | 118378125 | − |
| | | | Antisense) | | | | |
| SEQ ID NO. 1385 | 4019418 | CXorf56 | Non-coding (UTR) | chrX | 118672165 | 118672189 | − |
| SEQ ID NO. 1386 | 4019610 | NDUFA1 | Non-coding (Exonic | chrX | 119010525 | 119010608 | − |
| | | | Antisense) | | | | |
| SEQ ID NO. 1387 | 4020462 | THOC2 | Non- | chrX | 122752083 | 122752388 | − |
| | | | coding (Intronic) | | | | |
| SEQ ID NO. 1388 | 4024375 | CDR1 | Non-coding (UTR) | chrX | 139865543 | 139865573 | − |
| SEQ ID NO. 1389 | 4024376 | CDR1 | Non-coding (UTR) | chrX | 139865644 | 139865739 | − |
| SEQ ID NO. 1390 | 4024377 | CDR1 | Coding | chrX | 139865828 | 139865852 | − |
| SEQ ID NO. 1391 | 4024378 | CDR1 | Coding | chrX | 139865883 | 139865921 | − |
| SEQ ID NO. 1392 | 4024379 | CDR1 | Coding | chrX | 139865939 | 139865971 | − |
| SEQ ID NO. 1393 | 4030986 | NCRNA00185 | Non-coding (Intronic | chrY | 21152685 | 21153560 | + |
| | | | Antisense) | | | | |
| SEQ ID NO. 1394 | 4034997 | DDX3Y | Non-coding (Exonic | chrY | 15030062 | 15030179 | − |
| | | | Antisense) | | | | |
| SEQ ID NO. 1395 | 4035834 | NCRNA00185 | Non- | chrY | 21152548 | 21152618 | − |
| | | | coding (Intronic) | | | | |
| SEQ ID NO. 1396 | 4035835 | NCRNA00185 | Non- | chrY | 21152684 | 21152813 | − |
| | | | coding (Intronic) | | | | |
| SEQ ID NO. 1397 | 4035838 | NCRNA00185 | Non- | chrY | 21154041 | 21154065 | − |
| | | | coding (Intronic) | | | | |
| SEQ ID NO. 1398 | 4035839 | CD24P4 | Non- | chrY | 21154244 | 21154350 | − |
| | | | coding (ncTranscript) | | | | |
| SEQ ID NO. 1399 | 4040526 | ANAPCI1 | Non-coding (UTR) | chr17 | 79858058 | 79858126 | + |
| SEQ ID NO. 1400 | 4041367 | P4HB | Coding | chr17 | 79813023 | 79813113 | − |
| SEQ ID NO. 1401 | 4041822 | DVL1 | Non-coding (Exonic | chr1 | 1270724 | 1270751 | + |
| | | | Antisense) | | | | |
| SEQ ID NO. 1402 | 4043125 | MIA3 | Non-coding (UTR) | chr1 | 222839167 | 222839194 | + |
| SEQ ID NO. 1403 | 4044199 | MRPL20 | Non-coding (UTR) | chr1 | 1337315 | 1337437 | − |
| SEQ ID NO. 1404 | 4044413 | PNRC2 | Non-coding (Exonic | chr1 | 24289858 | 24289900 | − |
| | | | Antisense) | | | | |
| SEQ ID NO. 1405 | 4047577 | RNF4 | Non-coding (UTR) | chr4 | 2517539 | 2517569 | + |
| SEQ ID NO. 1406 | 4052399 | SNRNP35 | Non- | chr12 | 123956765 | 123956890 | + |
| | | | coding (ncTranscript) | | | | |
| SEQ ID NO. 1407 | 4055295 | ATRX | Coding | chrX | 76939261 | 76939355 | − |
| SEQ ID NO. 1408 | 4055302 | ATRX | Coding | chrX | 76937669 | 76938071 | − |
| SEQ ID NO. 1409 | 2720180 | LAP3 | Non-coding (UTR) | chr4 | 17609540 | 17609571 | + |
| SEQ ID NO. 1410 | 2347642 | SLC44A3 | Coding | chr1 | 95294064 | 95294116 | + |
| SEQ ID NO. 1411 | 2395344 | RERE | Non- | chr1 | 8626188 | 8626254 | − |
| | | | coding (Intronic) | | | | |
| SEQ ID NO. 1412 | 2397069 | DHRS3 | Non- | chr1 | 12675869 | 12676175 | − |
| | | | coding (Intronic) | | | | |
| SEQ ID NO. 1413 | 2430377 | AL122007.1 | Non-coding (Exonic | chr1 | 118406131 | 118406652 | − |
| | | | Antisense) | | | | |
| SEQ ID NO. 1414 | 2448270 | TPR | Coding | chr1 | 186302264 | 186302484 | − |
| SEQ ID NO. 1415 | 2553581 | EML6 | Non-coding (Exonic | chr2 | 55198361 | 55198412 | − |
| | | | Antisense) | | | | |
| SEQ ID NO. 1416 | 2600700 | EPHA4 | Non-coding (UTR) | chr2 | 222290193 | 222290664 | − |
| SEQ ID NO. 1417 | 2624519 | CACNA1D | Coding | chr3 | 53839020 | 53839140 | + |
| SEQ ID NO. 1418 | 2651846 | GPR160 | Non- | chr3 | 169760241 | 169760280 | + |
| | | | coding (Intronic) | | | | |
| SEQ ID NO. 1419 | 2682441 | RYBP | Coding | chr3 | 72428191 | 72428278 | − |
| SEQ ID NO. 1420 | 2704512 | MECOM | Non- | chr3 | 169039906 | 169040029 | − |
| | | | coding (Intronic) | | | | |
| SEQ ID NO. 1421 | 2704702 | MECOM | Non- | chr3 | 169245434 | 169245479 | − |
| | | | coding (Intronic) | | | | |
| SEQ ID NO. 1422 | 2902832 | C2, XXbac- | Coding | chr6 | 31912504 | 31912533 | + |
| | | BPG116M5.17, CFB | | | | | |
| SEQ ID NO. 1423 | 2918558 | RP11-98I9.4 | Non- | chr6 | 99874240 | 99874405 | + |
| | | | coding (Intronic) | | | | |
| SEQ ID NO. 1424 | 3031661 | ABP1 | Non- | chr7 | 150548944 | 150549009 | + |
| | | | coding (Intronic) | | | | |
| SEQ ID NO. 1425 | 3212761 | AGTPBP1 | Non- | chr9 | 88262440 | 88262595 | − |
| | | | coding (Intronic) | | | | |
| SEQ ID NO. 1426 | 3268183 | TACC2 | Non- | chr10 | 123995651 | 123995692 | + |
| | | | coding (Intronic) | | | | |
| SEQ ID NO. 1427 | 3472753 | chr12−: 115105073-115105114 | Non- | chr12 | 115105073 | 115105114 | − |
| | | | coding (Intergenic) | | | | |
| SEQ ID NO. 1428 | 3501557 | ANKRD10 | Non-coding (Intronic | chr13 | 111551671 | 111552005 | + |
| | | | Antisense) | | | | |
| SEQ ID NO. 1429 | 3543621 | NUMB | Non-coding (Intronic | chr14 | 73924866 | 73924936 | + |
| | | | Antisense) | | | | |
| SEQ ID NO. 1430 | 3551833 | YY1 | Non- | chr14 | 100731828 | 100732634 | + |
| | | | coding (Intronic) | | | | |
| SEQ ID NO. 1431 | 3562041 | PNN | Non-coding (Exonic | chr14 | 39650300 | 39650740 | − |
| | | | Antisense) | | | | |
| SEQ ID NO. 1432 | 3661099 | RBL2 | Coding | chr16 | 53514479 | 53514525 | + |
| SEQ ID NO. 1433 | 3667907 | DHX38 | Coding | chr16 | 72131593 | 72131680 | + |
| SEQ ID NO. 1434 | 3701840 | MPHOSPH6 | Non-coding (UTR) | chr16 | 82182086 | 82182335 | − |
| SEQ ID NO. 1435 | 3840609 | ZNF347 | Non-coding (Intronic | chr19 | 53653851 | 53653898 | + |
| | | | Antisense) | | | | |
| SEQ ID NO. 1436 | 3889144 | ATP9A | Non-coding (Exonic | chr20 | 50217042 | 50217277 | + |
| | | | Antisense) | | | | |
| SEQ ID NO. 1437 | 3889625 | TSHZ2, AL354993.1 | Coding | chr20 | 52105591 | 52105740 | + |
| SEQ ID NO. 1438 | 3897081 | PLCB1 | Non-coding (Intronic | chr20 | 8419858 | 8419910 | − |
| | | | Antisense) | | | | |
| SEQ ID NO. 1439 | 3902441 | DEFB121 | Coding | chr20 | 29993912 | 29993949 | − |
| SEQ ID NO. 1440 | 4026042 | GABRE | Non- | chrX | 151140414 | 151140607 | − |
| | | | coding (ncTranscript) | | | | |

1,425 prognostic markers for MIBC recurrence. For each marker, the Affymetrix Probeset ID, associated gene, category and chromosomal location based on hg19 human genome version is indicated.

TABLE 17

| | Benjamini |
|---|---|
| Term | P-value |
| GO: 0016071~mRNA metabolic process | 0.000000 |
| GO: 0006396~RNA processing | 0.000000 |
| GO: 0006412~translation | 0.000000 |
| GO: 0006397~mRNA processing | 0.000000 |
| GO: 0046907~intracellular transport | 0.000000 |
| GO: 0008380~RNA splicing | 0.000000 |
| GO: 0006414~translational elongation | 0.000000 |
| GO: 0015031~protein transport | 0.000004 |
| GO: 0045184~establishment of protein | 0.000006 |
| localization | |
| GO: 0006403~RNA localization | 0.00005 |
| GO: 0051168~nuclear export | 0.00007 |
| GO: 0000377~RNA splicing, via | 0.00007 |
| transesterification reactions with bulged | |
| adenosine as nucleophile | |
| GO: 0000375~RNA splicing, via | 0.00007 |
| transesterification reactions | |
| GO: 0000398~nuclear mRNA splicing, via | 0.00007 |
| spliceosome | |
| GO: 0008104~protein localization | 0.00007 |
| GO: 0006913~nucleocytoplasmic transport | 0.00009 |
| GO: 0051236~establishment of RNA | 0.00009 |
| localization | |
| GO: 0050657~nucleic acid transport | 0.00009 |
| GO: 0050658~RNA transport | 0.00009 |
| GO: 0007049~cell cycle | 0.00009 |
| GO: 0051169~nuclear transport | 0.0001 |
| GO: 0006405~RNA export from nucleus | 0.0001 |
| GO: 0006886~intracellular protein | 0.0003 |
| transport | |
| GO: 0044265~cellular macromolecule | 0.0004 |
| catabolic process | |
| GO: 0070727~cellular macromolecule | 0.0007 |
| localization | |
| GO: 0015931~nucleobase, nucleoside, | 0.0007 |
| nucleotide and nucleic acid transport | |
| GO: 0009057~macromolecule catabolic | 0.001 |
| process | |
| GO: 0034613~cellular protein localization | 0.001 |
| GO: 0022402~cell cycle process | 0.001 |
| GO: 0000278~mitotic cell cycle | 0.002 |
| GO: 0051028~mRNA transport | 0.004 |
| GO: 0009141~nucleoside triphosphate | 0.005 |
| metabolic process | |
| GO: 0006605~protein targeting | 0.01 |
| GO: 0022613~ribonucleoprotein complex | 0.01 |
| biogenesis | |
| GO: 0009142~nucleoside triphosphate | 0.01 |
| biosynthetic process | |
| GO: 0043933~macromolecular complex | 0.01 |
| subunit organization | |
| GO: 0065003~macromolecular complex | 0.01 |
| assembly | |
| GO: 0006259~DNA metabolic process | 0.01 |
| GO: 0051276~chromosome organization | 0.01 |
| GO: 0009201~ribonucleoside triphosphate | 0.01 |
| biosynthetic process | |
| GO: 0006754~ATP biosynthetic process | 0.01 |
| GO: 0006406~mRNA export from nucleus | 0.02 |
| GO: 0046034~ATP metabolic process | 0.02 |
| GO: 0006325~chromatin organization | 0.02 |
| GO: 0006511~ubiquitin-dependent protein | 0.02 |
| catabolic process | |
| GO: 0009199~ribonucleoside triphosphate | 0.03 |
| metabolic process | |
| GO: 0043631~RNA polyadenylation | 0.03 |
| GO: 0016568~chromatin modification | 0.03 |
| GO:0006119~oxidative phosphorylation | 0.03 |
| GO: 0009206~purine ribonucleoside | 0.03 |
| triphosphate biosynthetic process | |
| GO: 0006974~response to DNA damage | 0.03 |
| stimulus | |
| GO: 0010605~negative regulation of | 0.03 |
| macromolecule metabolic process | |
| GO:0009144~purine nucleoside | 0.03 |
| triphosphate metabolic process | |
| GO: 0033554~cellular response to stress | 0.03 |
| GO: 0009145~purine nucleoside | 0.03 |
| triphosphate biosynthetic process | |
| GO: 0070647~protein modification by | 0.03 |
| small protein conjugation or removal | |
| GO: 0051603~proteolysis involved in | 0.03 |
| cellular protein catabolic process | |
| GO: 0009260~ribonucleotide biosynthetic | 0.03 |
| process | |
| GO: 0006892~post-Golgi vesicle-mediated | 0.04 |
| transport | |
| GO: 0030518~steroid hormone receptor | 0.04 |
| signaling pathway | |
| GO: 0044257~cellular protein catabolic | 0.04 |
| process | |
| GO: 0022618~ribonucleoprotein complex | 0.04 |
| assembly | |
| GO: 0034220~ion transmembrane transport | 0.04 |
| GO: 0030163~protein catabolic process | 0.04 |
| GO: 0015985~energy coupled proton | 0.04 |
| transport, down electrochemical gradient | |
| GO: 0015986~ATP synthesis coupled | 0.04 |
| proton transport | |
| GO: 0006457~protein folding | 0.04 |
| GO: 0006091~generation of precursor | 0.04 |
| metabolites and energy | |
| GO: 0022403~cell cycle phase | 0.04 |
| GO: 0006461~protein complex assembly | 0.04 |
| GO: 0070271~protein complex biogenesis | 0.04 |
| GO: 0009152~purine ribonucleotide | 0.04 |
| biosynthetic process | |
| GO: 0009205~purine ribonucleoside | 0.04 |
| triphosphate metabolic process | |

Gene Ontology enrichment of the prognostic markers selected. P-values shown are after correction for multiple testing using Benjamini-Hochberg method.

TABLE 18

| Affymetrix | | | | | | | | | | | | | | | |
|---|---|---|---|---|---|---|---|---|---|---|---|---|---|---|---|
| Probeset | | WILCOX | | | ACCURACY | | | | | | KM | UVA HR | UVA OR | MVA HR | MVA OR |
| ID | AUC | P-VALUE | MFD | ACCURACY | P-VALUE | SENSITIVITY | SPECIFICITY | PPV | NPV | CUTOFF | P-VALUE | P-VALUE | P-VALUE | P-VALUE | P-VALUE |
| 2318654 | 0.61 | 0.0104 | 1.28 | 0.61 | 0.0028 | 0.64 | 0.57 | 0.61 | 0.61 | 7.84 | 0.0046 | 0.0149 | 0.0103 | 0.2 | 0.022 |
| 2318755 | 0.62 | 0.0034 | 1.53 | 0.64 | 0.0001 | 0.6 | 0.68 | 0.66 | 0.63 | 5.42 | 0.0001 | 0.0027 | 0.0029 | 0.02 | 0.006 |
| 2320062 | 0.62 | 0.003 | 1.26 | 0.62 | 0.0011 | 0.62 | 0.61 | 0.62 | 0.61 | 4.99 | 0.0006 | 0.0029 | 0.0045 | 0.02 | 0.008 |
| 2321661 | 0.62 | 0.0034 | 1.34 | 0.62 | 0.0007 | 0.65 | 0.59 | 0.62 | 0.62 | 6.56 | 0.0021 | 0.0045 | 0.0025 | 0.05 | 0.006 |
| 2322383 | 0.61 | 0.0074 | 1.35 | 0.6 | 0.0042 | 0.6 | 0.6 | 0.61 | 0.6 | 5.28 | 0.0032 | 0.0095 | 0.009 | 0.05 | 0.019 |
| 2325809 | 0.61 | 0.0052 | 1.29 | 0.62 | 0.0011 | 0.63 | 0.6 | 0.62 | 0.61 | 6.05 | 0.001 | 0.0064 | 0.0056 | 0.15 | 0.046 |
| 2327494 | 0.62 | 0.0039 | 1.35 | 0.62 | 0.0011 | 0.63 | 0.6 | 0.62 | 0.61 | 6.48 | 0.0017 | 0.0027 | 0.0022 | 0.06 | 0.005 |
| 2327865 | 0.63 | 0.002 | 1.43 | 0.61 | 0.0018 | 0.65 | 0.57 | 0.61 | 0.62 | 5.32 | 0.0011 | 0.0014 | 0.0016 | 0.08 | 0.013 |
| 2328504 | 0.63 | 0.0019 | 1.31 | 0.63 | 0.0002 | 0.64 | 0.62 | 0.64 | 0.63 | 6.57 | 0.0006 | 0.0032 | 0.0013 | 0.04 | 0.003 |
| 2328507 | 0.64 | 0.0009 | 1.29 | 0.59 | 0.0138 | 0.46 | 0.72 | 0.63 | 0.56 | 5.28 | 0.009 | 0.004 | 0.0027 | 0.21 | 0.025 |
| 2329019 | 0.61 | 0.0089 | 1.29 | 0.61 | 0.0018 | 0.61 | 0.61 | 0.62 | 0.61 | 6.44 | 0.0011 | 0.0074 | 0.0071 | 0.11 | 0.014 |
| 2329798 | 0.62 | 0.004 | 1.45 | 0.6 | 0.0042 | 0.55 | 0.65 | 0.62 | 0.59 | 4.91 | 0.0066 | 0.0086 | 0.0065 | 0.1 | 0.053 |
| 2330704 | 0.6 | 0.0122 | 1.32 | 0.58 | 0.0276 | 0.59 | 0.56 | 0.58 | 0.57 | 5.04 | 0.0429 | 0.0159 | 0.0114 | 0.13 | 0.022 |
| 2331419 | 0.62 | 0.0043 | 1.28 | 0.58 | 0.0197 | 0.49 | 0.68 | 0.61 | 0.56 | 4.59 | 0.0151 | 0.012 | 0.0062 | 0.87 | 0.12 |
| 2331842 | 0.61 | 0.0103 | 1.32 | 0.6 | 0.0042 | 0.54 | 0.66 | 0.63 | 0.59 | 6 | 0.0052 | 0.009 | 0.0077 | 0.22 | 0.065 |
| 2332192 | 0.65 | 0.0004 | 1.6 | 0.61 | 0.0018 | 0.57 | 0.65 | 0.63 | 0.6 | 4.91 | 0.0009 | 0.0005 | 0.0005 | 0.06 | 0.011 |
| 2332740 | 0.64 | 0.0006 | 1.31 | 0.61 | 0.0018 | 0.5 | 0.72 | 0.65 | 0.59 | 3.65 | 0.001 | 0.0007 | 0.0014 | 0.02 | 0.004 |
| 2334302 | 0.62 | 0.0044 | 1.31 | 0.62 | 0.0011 | 0.62 | 0.61 | 0.62 | 0.61 | 6.23 | 0.0023 | 0.0051 | 0.0035 | 0.12 | 0.008 |
| 2336960 | 0.64 | 0.0005 | 1.26 | 0.62 | 0.0007 | 0.61 | 0.63 | 0.63 | 0.61 | 6.56 | 0.0005 | 0.0009 | 0.0006 | 0.08 | 0.009 |
| 2339310 | 0.61 | 0.0055 | 1.35 | 0.61 | 0.0018 | 0.53 | 0.69 | 0.64 | 0.59 | 4.5 | 0.0017 | 0.0071 | 0.0062 | 0.15 | 0.028 |
| 2342617 | 0.62 | 0.0034 | 1.27 | 0.58 | 0.0276 | 0.45 | 0.71 | 0.62 | 0.56 | 4.65 | 0.0382 | 0.0211 | 0.0088 | 0.15 | 0.012 |
| 2345634 | 0.64 | 0.0006 | 1.36 | 0.6 | 0.0064 | 0.62 | 0.57 | 0.6 | 0.6 | 4.71 | 0.0046 | 0.0003 | 0.0005 | 0.02 | 0.002 |
| 2345663 | 0.67 | 0 | 1.25 | 0.64 | 0.0001 | 0.68 | 0.59 | 0.63 | 0.64 | 4.43 | 0.0006 | 0 | 0 | 0.03 | 0.001 |
| 2345666 | 0.61 | 0.0092 | 1.33 | 0.6 | 0.0064 | 0.53 | 0.66 | 0.62 | 0.58 | 4.06 | 0.0021 | 0.0116 | 0.0152 | 0.16 | 0.101 |
| 2346911 | 0.62 | 0.0041 | 1.34 | 0.62 | 0.0011 | 0.55 | 0.68 | 0.64 | 0.6 | 7.34 | 0.0011 | 0.0042 | 0.0043 | 0.51 | 0.118 |
| 2346912 | 0.63 | 0.0017 | 1.34 | 0.56 | 0.0681 | 0.41 | 0.72 | 0.6 | 0.54 | 3.86 | 0.1027 | 0.0139 | 0.0051 | 0.23 | 0.032 |
| 2347108 | 0.62 | 0.004 | 1.33 | 0.6 | 0.0064 | 0.51 | 0.68 | 0.63 | 0.58 | 5.73 | 0.0064 | 0.0068 | 0.0049 | 0.13 | 0.036 |
| 2348819 | 0.63 | 0.0014 | 1.33 | 0.63 | 0.0002 | 0.63 | 0.63 | 0.64 | 0.63 | 5.92 | 0.0002 | 0.0019 | 0.0013 | 0.14 | 0.011 |
| 2350305 | 0.62 | 0.0032 | 1.31 | 0.63 | 0.0004 | 0.63 | 0.62 | 0.63 | 0.62 | 5.19 | 0.0005 | 0.0026 | 0.0025 | 0.26 | 0.085 |
| 2350371 | 0.67 | 0.0001 | 1.28 | 0.61 | 0.0028 | 0.47 | 0.76 | 0.66 | 0.58 | 4.38 | 0.0012 | 0.0002 | 0.0001 | 0.05 | 0.016 |
| 2352268 | 0.63 | 0.0015 | 1.27 | 0.61 | 0.0028 | 0.57 | 0.64 | 0.62 | 0.59 | 6.23 | 0.0028 | 0.0026 | 0.0032 | 0.06 | 0.01 |
| 2353492 | 0.6 | 0.0139 | 1.25 | 0.59 | 0.0095 | 0.61 | 0.57 | 0.6 | 0.59 | 5.83 | 0.0151 | 0.0102 | 0.0144 | 0.11 | 0.025 |
| 2358275 | 0.64 | 0.0009 | 1.25 | 0.62 | 0.0011 | 0.56 | 0.67 | 0.64 | 0.6 | 4.26 | 0.0009 | 0.001 | 0.0018 | 0.06 | 0.067 |
| 2358923 | 0.61 | 0.0058 | 1.39 | 0.65 | 0 | 0.69 | 0.6 | 0.64 | 0.66 | 6.94 | 0 | 0.0052 | 0.0046 | 0.07 | 0.009 |
| 2359857 | 0.65 | 0.0003 | 1.32 | 0.63 | 0.0004 | 0.6 | 0.65 | 0.64 | 0.62 | 4.36 | 0.0006 | 0.0009 | 0.0005 | 0.03 | 0.008 |
| 2360078 | 0.65 | 0.0003 | 1.29 | 0.61 | 0.0018 | 0.54 | 0.68 | 0.64 | 0.59 | 7.33 | 0.0018 | 0.0003 | 0.0005 | 0.03 | 0.002 |
| 2360119 | 0.64 | 0.0006 | 1.26 | 0.65 | 0 | 0.64 | 0.65 | 0.66 | 0.64 | 7.33 | 0.0001 | 0.0005 | 0.0005 | 0.02 | 0.004 |
| 2360120 | 0.65 | 0.0002 | 1.25 | 0.61 | 0.0018 | 0.61 | 0.61 | 0.62 | 0.61 | 7.06 | 0.001 | 0.0001 | 0.0002 | 0.01 | 0.001 |
| 2360340 | 0.64 | 0.0004 | 1.26 | 0.65 | 0 | 0.57 | 0.72 | 0.68 | 0.62 | 5.7 | 0 | 0.0004 | 0.0006 | 0.02 | 0.012 |
| 2360716 | 0.61 | 0.0054 | 1.26 | 0.62 | 0.0011 | 0.7 | 0.53 | 0.61 | 0.63 | 7.26 | 0.0018 | 0.0082 | 0.0061 | 0.12 | 0.016 |
| 2360962 | 0.64 | 0.0005 | 1.34 | 0.64 | 0.0001 | 0.61 | 0.66 | 0.65 | 0.63 | 5.34 | 0.0001 | 0.0004 | 0.0005 | 0.02 | 0.003 |
| 2361031 | 0.61 | 0.0082 | 1.27 | 0.59 | 0.0138 | 0.51 | 0.66 | 0.61 | 0.57 | 3.26 | 0.0065 | 0.0304 | 0.025 | 0.12 | 0.06 |
| 2363064 | 0.64 | 0.0006 | 1.27 | 0.62 | 0.0011 | 0.62 | 0.61 | 0.62 | 0.61 | 6.56 | 0.0014 | 0.0006 | 0.0005 | 0.04 | 0.003 |
| 2363334 | 0.6 | 0.0138 | 1.25 | 0.56 | 0.0681 | 0.54 | 0.58 | 0.57 | 0.55 | 5.05 | 0.0524 | 0.0144 | 0.0146 | 0.43 | 0.19 |
| 2363956 | 0.63 | 0.0012 | 1.26 | 0.61 | 0.0018 | 0.51 | 0.71 | 0.65 | 0.59 | 4.39 | 0.0046 | 0.0069 | 0.0039 | 0.27 | 0.052 |
| 2365991 | 0.61 | 0.0091 | 1.48 | 0.59 | 0.0138 | 0.55 | 0.62 | 0.6 | 0.58 | 4.71 | 0.0136 | 0.0237 | 0.0154 | 0.16 | 0.044 |
| 2367178 | 0.65 | 0.0002 | 1.41 | 0.64 | 0.0001 | 0.61 | 0.66 | 0.65 | 0.63 | 5.15 | 0.0001 | 0.0006 | 0.0004 | 0.06 | 0.005 |
| 2367214 | 0.61 | 0.0062 | 1.42 | 0.6 | 0.0064 | 0.61 | 0.58 | 0.6 | 0.59 | 4.86 | 0.0052 | 0.0079 | 0.0065 | 0.1 | 0.017 |
| 2367236 | 0.6 | 0.0119 | 1.3 | 0.61 | 0.0028 | 0.6 | 0.61 | 0.62 | 0.6 | 5.7 | 0.0027 | 0.0067 | 0.0095 | 0.37 | 0.135 |
| 2369830 | 0.65 | 0.0002 | 1.41 | 0.59 | 0.0138 | 0.54 | 0.63 | 0.6 | 0.57 | 4.65 | 0.0121 | 0.0008 | 0.0008 | 0.07 | 0.021 |
| 2370014 | 0.63 | 0.0022 | 1.27 | 0.6 | 0.0064 | 0.57 | 0.62 | 0.61 | 0.59 | 4.41 | 0.0047 | 0.001 | 0.0021 | 0.13 | 0.058 |
| 2371291 | 0.66 | 0.0001 | 1.27 | 0.6 | 0.0042 | 0.5 | 0.7 | 0.64 | 0.58 | 5.49 | 0.0025 | 0.0001 | 0.0003 | 0.04 | 0.018 |
| 2371339 | 0.63 | 0.0021 | 1.34 | 0.6 | 0.0064 | 0.54 | 0.65 | 0.62 | 0.58 | 4.45 | 0.0045 | 0.012 | 0.0094 | 0.1 | 0.026 |
| 2371796 | 0.65 | 0.0002 | 1.31 | 0.59 | 0.0095 | 0.5 | 0.68 | 0.62 | 0.57 | 5.76 | 0.004 | 0.0003 | 0.0005 | 0.02 | 0.005 |
| 2373002 | 0.62 | 0.0042 | 1.38 | 0.6 | 0.0042 | 0.51 | 0.69 | 0.63 | 0.58 | 4.06 | 0.0014 | 0.02 | 0.019 | 0.05 | 0.025 |
| 2375035 | 0.65 | 0.0002 | 1.41 | 0.62 | 0.0007 | 0.51 | 0.73 | 0.67 | 0.6 | 4.75 | 0.0002 | 0.0001 | 0.0001 | 0.02 | 0.002 |
| 2375037 | 0.65 | 0.0002 | 1.25 | 0.62 | 0.0007 | 0.59 | 0.65 | 0.64 | 0.61 | 6.97 | 0.0013 | 0.0003 | 0.0002 | 0.05 | 0.003 |
| 2375675 | 0.63 | 0.0014 | 1.37 | 0.62 | 0.0011 | 0.59 | 0.64 | 0.63 | 0.61 | 5.35 | 0.0024 | 0.0035 | 0.0013 | 0.18 | 0.014 |
| 2375780 | 0.61 | 0.0102 | 1.36 | 0.58 | 0.0197 | 0.53 | 0.63 | 0.6 | 0.57 | 6.05 | 0.0192 | 0.0159 | 0.012 | 0.15 | 0.013 |
| 2375850 | 0.64 | 0.0006 | 1.26 | 0.64 | 0.0001 | 0.65 | 0.63 | 0.65 | 0.64 | 6.63 | 0.0001 | 0.0008 | 0.0009 | 0.09 | 0.014 |
| 2375890 | 0.61 | 0.0058 | 1.28 | 0.6 | 0.0042 | 0.67 | 0.53 | 0.6 | 0.61 | 6.77 | 0.0035 | 0.0059 | 0.0046 | 0.11 | 0.009 |
| 2377473 | 0.65 | 0.0004 | 1.34 | 0.6 | 0.0064 | 0.5 | 0.69 | 0.63 | 0.58 | 5.02 | 0.0034 | 0.0007 | 0.0008 | 0.05 | 0.003 |
| 2377508 | 0.64 | 0.0005 | 1.52 | 0.63 | 0.0002 | 0.68 | 0.58 | 0.63 | 0.64 | 5.81 | 0.0001 | 0.0003 | 0.0005 | 0.01 | 0.002 |
| 2378615 | 0.61 | 0.0057 | 1.29 | 0.57 | 0.0513 | 0.47 | 0.67 | 0.59 | 0.55 | 3.72 | 0.0199 | 0.0089 | 0.0238 | 0.03 | 0.072 |
| 2379787 | 0.62 | 0.0024 | 1.3 | 0.62 | 0.0007 | 0.71 | 0.53 | 0.61 | 0.64 | 4.58 | 0.0012 | 0.0041 | 0.003 | 0.2 | 0.038 |
| 2383763 | 0.62 | 0.0045 | 1.3 | 0.6 | 0.0064 | 0.59 | 0.6 | 0.61 | 0.59 | 6.42 | 0.0072 | 0.0039 | 0.0049 | 0.06 | 0.013 |
| 2389076 | 0.62 | 0.0028 | 1.34 | 0.61 | 0.0018 | 0.53 | 0.69 | 0.64 | 0.59 | 4.92 | 0.0007 | 0.0025 | 0.0032 | 0.18 | 0.014 |
| 2389079 | 0.64 | 0.0006 | 1.29 | 0.6 | 0.0064 | 0.5 | 0.7 | 0.63 | 0.58 | 4.6 | 0.0082 | 0.001 | 0.0011 | 0.05 | 0.01 |
| 2389084 | 0.61 | 0.0105 | 1.27 | 0.62 | 0.0011 | 0.48 | 0.77 | 0.68 | 0.59 | 5.19 | 0.0002 | 0.0033 | 0.0064 | 0.05 | 0.031 |
| 2389086 | 0.64 | 0.0004 | 1.27 | 0.6 | 0.0064 | 0.54 | 0.65 | 0.62 | 0.58 | 5.44 | 0.0037 | 0.0005 | 0.0007 | 0.03 | 0.02 |
| 2389817 | 0.65 | 0.0002 | 1.3 | 0.61 | 0.0018 | 0.55 | 0.67 | 0.64 | 0.59 | 7.1 | 0.0021 | 0.0008 | 0.0007 | 0.05 | 0.004 |
| 2394560 | 0.61 | 0.0062 | 1.62 | 0.61 | 0.0028 | 0.63 | 0.58 | 0.61 | 0.61 | 5.1 | 0.002 | 0.0071 | 0.0053 | 0.07 | 0.01 |
| 2395192 | 0.62 | 0.0035 | 1.35 | 0.61 | 0.0018 | 0.53 | 0.69 | 0.64 | 0.59 | 6.14 | 0.0007 | 0.0013 | 0.0023 | 0.01 | 0.005 |
| 2395195 | 0.64 | 0.0006 | 1.26 | 0.62 | 0.0007 | 0.52 | 0.72 | 0.66 | 0.6 | 5.43 | 0.0002 | 0.001 | 0.0015 | 0.01 | 0.004 |
| 2396421 | 0.63 | 0.0019 | 1.27 | 0.61 | 0.0018 | 0.6 | 0.62 | 0.62 | 0.6 | 4.26 | 0.0007 | 0.0024 | 0.0032 | 0 | 0.003 |
| 2400180 | 0.63 | 0.0016 | 1.29 | 0.61 | 0.0018 | 0.5 | 0.72 | 0.65 | 0.59 | 6.32 | 0.0014 | 0.0008 | 0.0012 | 0.07 | 0.022 |
| 2400323 | 0.63 | 0.0011 | 1.3 | 0.58 | 0.0276 | 0.43 | 0.73 | 0.62 | 0.55 | 4.31 | 0.0167 | 0.0015 | 0.0025 | 0.1 | 0.041 |
| 2401277 | 0.65 | 0.0002 | 1.26 | 0.62 | 0.0007 | 0.52 | 0.72 | 0.66 | 0.6 | 2.91 | 0.0004 | 0.0002 | 0.0005 | 0.01 | 0 |
| 2401288 | 0.64 | 0.0005 | 1.26 | 0.63 | 0.0004 | 0.69 | 0.56 | 0.62 | 0.64 | 5.91 | 0.0002 | 0.0005 | 0.0007 | 0.01 | 0.002 |
| 2401350 | 0.65 | 0.0004 | 1.37 | 0.63 | 0.0002 | 0.63 | 0.63 | 0.64 | 0.63 | 5.21 | 0.0001 | 0.0005 | 0.0005 | 0.02 | 0.002 |
| 2401368 | 0.65 | 0.0002 | 1.38 | 0.65 | 0 | 0.59 | 0.71 | 0.68 | 0.63 | 4.69 | 0 | 0 | 0.0001 | 0.02 | 0.021 |
| 2402210 | 0.64 | 0.0004 | 1.25 | 0.62 | 0.0007 | 0.57 | 0.67 | 0.64 | 0.61 | 5.89 | 0.0004 | 0.0012 | 0.0012 | 0.04 | 0.006 |
| 2405379 | 0.63 | 0.0013 | 1.35 | 0.61 | 0.0028 | 0.58 | 0.63 | 0.62 | 0.6 | 5.61 | 0.0026 | 0.0017 | 0.0016 | 0.03 | 0.008 |
| 2406094 | 0.63 | 0.0018 | 1.43 | 0.62 | 0.0007 | 0.6 | 0.64 | 0.64 | 0.61 | 6.24 | 0.0003 | 0.0014 | 0.0019 | 0.08 | 0.015 |
| 2406148 | 0.6 | 0.0136 | 1.36 | 0.63 | 0.0004 | 0.71 | 0.54 | 0.62 | 0.65 | 5.88 | 0.0009 | 0.0208 | 0.0132 | 0.22 | 0.034 |
| 2407134 | 0.61 | 0.0063 | 1.35 | 0.61 | 0.0028 | 0.56 | 0.65 | 0.63 | 0.59 | 4.55 | 0.0013 | 0.0034 | 0.0046 | 0.08 | 0.027 |
| 2408880 | 0.61 | 0.0062 | 1.26 | 0.58 | 0.0276 | 0.49 | 0.67 | 0.6 | 0.56 | 4.83 | 0.0153 | 0.0024 | 0.0069 | 0.02 | 0.024 |
| 2410112 | 0.63 | 0.0015 | 1.38 | 0.6 | 0.0042 | 0.66 | 0.54 | 0.6 | 0.61 | 5.4 | 0.0056 | 0.0016 | 0.001 | 0.02 | 0.002 |
| 2410528 | 0.62 | 0.0043 | 1.29 | 0.62 | 0.0007 | 0.72 | 0.52 | 0.61 | 0.65 | 6.06 | 0.0004 | 0.0046 | 0.0058 | 0.05 | 0.011 |
| 2412669 | 0.62 | 0.0038 | 1.35 | 0.59 | 0.0095 | 0.53 | 0.65 | 0.61 | 0.58 | 4.74 | 0.0137 | 0.0096 | 0.0064 | 0.1 | 0.019 |
| 2413580 | 0.63 | 0.0015 | 1.27 | 0.6 | 0.0042 | 0.53 | 0.67 | 0.63 | 0.58 | 5.1 | 0.0023 | 0.0068 | 0.0044 | 0.12 | 0.021 |
| 2414030 | 0.63 | 0.0015 | 1.43 | 0.61 | 0.0028 | 0.55 | 0.66 | 0.63 | 0.59 | 4.8 | 0.0039 | 0.0031 | 0.002 | 0.04 | 0.007 |
| 2414960 | 0.64 | 0.001 | 1.4 | 0.59 | 0.0138 | 0.54 | 0.63 | 0.6 | 0.57 | 4.87 | 0.0112 | 0.0005 | 0.0014 | 0.02 | 0.012 |
| 2417788 | 0.61 | 0.0067 | 1.29 | 0.6 | 0.0042 | 0.54 | 0.66 | 0.63 | 0.59 | 4.95 | 0.0017 | 0.0071 | 0.0069 | 0.19 | 0.047 |
| 2418028 | 0.62 | 0.0035 | 1.28 | 0.64 | 0.0001 | 0.6 | 0.68 | 0.66 | 0.63 | 5.64 | 0.0001 | 0.0028 | 0.0029 | 0.08 | 0.017 |
| 2419242 | 0.65 | 0.0002 | 1.35 | 0.6 | 0.0042 | 0.46 | 0.76 | 0.66 | 0.57 | 4.58 | 0.002 | 0.0006 | 0.0004 | 0.1 | 0.008 |
| 2419247 | 0.61 | 0.0076 | 1.3 | 0.6 | 0.0042 | 0.52 | 0.68 | 0.63 | 0.58 | 5.26 | 0.0031 | 0.0063 | 0.0047 | 0.54 | 0.073 |
| 2419276 | 0.65 | 0.0002 | 1.29 | 0.62 | 0.0007 | 0.64 | 0.6 | 0.63 | 0.62 | 5.81 | 0.0009 | 0.0004 | 0.0003 | 0.06 | 0.004 |
| 2420740 | 0.61 | 0.0086 | 1.39 | 0.57 | 0.038 | 0.52 | 0.62 | 0.59 | 0.56 | 4.21 | 0.0444 | 0.0104 | 0.0087 | 0.34 | 0.078 |
| 2420839 | 0.6 | 0.0113 | 1.3 | 0.62 | 0.0011 | 0.5 | 0.74 | 0.67 | 0.59 | 4.79 | 0.0006 | 0.0042 | 0.0044 | 0.18 | 0.079 |
| 2421273 | 0.64 | 0.0009 | 1.28 | 0.62 | 0.0007 | 0.54 | 0.7 | 0.65 | 0.6 | 6.21 | 0.0004 | 0.0009 | 0.0009 | 0.08 | 0.007 |
| 2421274 | 0.63 | 0.0013 | 1.32 | 0.63 | 0.0002 | 0.51 | 0.76 | 0.68 | 0.6 | 4.85 | 0.0001 | 0.0014 | 0.0015 | 0.25 | 0.034 |
| 2421932 | 0.61 | 0.006 | 1.27 | 0.61 | 0.0018 | 0.69 | 0.53 | 0.6 | 0.63 | 6.69 | 0.0041 | 0.0152 | 0.0062 | 0.27 | 0.014 |
| 2422519 | 0.62 | 0.0039 | 1.32 | 0.63 | 0.0004 | 0.57 | 0.68 | 0.65 | 0.61 | 4.68 | 0.0005 | 0.0052 | 0.004 | 0.18 | 0.02 |
| 2423200 | 0.65 | 0.0003 | 1.35 | 0.62 | 0.0007 | 0.62 | 0.62 | 0.63 | 0.62 | 7.02 | 0.0009 | 0.0003 | 0.0003 | 0.09 | 0.005 |
| 2427009 | 0.6 | 0.0108 | 1.45 | 0.59 | 0.0138 | 0.54 | 0.63 | 0.6 | 0.57 | 3.93 | 0.0112 | 0.0106 | 0.0105 | 0.04 | 0.01 |
| 2427501 | 0.64 | 0.001 | 1.48 | 0.6 | 0.0064 | 0.57 | 0.62 | 0.61 | 0.59 | 4.97 | 0.0075 | 0.0015 | 0.0015 | 0.11 | 0.013 |
| 2427978 | 0.63 | 0.0022 | 1.29 | 0.63 | 0.0004 | 0.62 | 0.63 | 0.64 | 0.62 | 6.01 | 0.0005 | 0.0032 | 0.0022 | 0.03 | 0.009 |
| 2428394 | 0.65 | 0.0003 | 1.31 | 0.65 | 0 | 0.59 | 0.7 | 0.67 | 0.63 | 6.29 | 0 | 0.0005 | 0.0003 | 0.05 | 0.004 |
| 2429071 | 0.62 | 0.0028 | 1.25 | 0.57 | 0.038 | 0.5 | 0.64 | 0.59 | 0.56 | 4.26 | 0.0596 | 0.0113 | 0.0054 | 0.05 | 0.006 |
| 2429119 | 0.65 | 0.0003 | 1.33 | 0.62 | 0.0011 | 0.57 | 0.66 | 0.64 | 0.6 | 5.45 | 0.0025 | 0.0008 | 0.0005 | 0.05 | 0.001 |
| 2429168 | 0.61 | 0.0075 | 1.39 | 0.57 | 0.0513 | 0.51 | 0.62 | 0.58 | 0.55 | 3.73 | 0.047 | 0.0176 | 0.0208 | 0.34 | 0.201 |
| 2429282 | 0.66 | 0.0001 | 1.32 | 0.62 | 0.0007 | 0.53 | 0.71 | 0.66 | 0.6 | 5 | 0.0005 | 0.0002 | 0.0002 | 0.04 | 0.007 |
| 2429309 | 0.63 | 0.0015 | 1.28 | 0.62 | 0.0011 | 0.62 | 0.61 | 0.62 | 0.61 | 6.71 | 0.0015 | 0.0011 | 0.001 | 0.11 | 0.009 |
| 2429310 | 0.64 | 0.0005 | 1.56 | 0.63 | 0.0004 | 0.65 | 0.6 | 0.63 | 0.63 | 5.66 | 0.0005 | 0.0005 | 0.0005 | 0.05 | 0.006 |
| 2433376 | 0.61 | 0.0063 | 1.29 | 0.64 | 0.0001 | 0.63 | 0.64 | 0.65 | 0.63 | 4.93 | 0.0001 | 0.0174 | 0.0114 | 0.08 | 0.013 |
| 2434721 | 0.63 | 0.001 | 1.26 | 0.6 | 0.0064 | 0.53 | 0.66 | 0.62 | 0.58 | 5.68 | 0.007 | 0.0015 | 0.0013 | 0.17 | 0.046 |
| 2435391 | 0.62 | 0.0031 | 1.27 | 0.61 | 0.0018 | 0.53 | 0.69 | 0.64 | 0.59 | 6.45 | 0.0006 | 0.0025 | 0.0049 | 0.01 | 0.01 |
| 2437095 | 0.61 | 0.0074 | 1.3 | 0.6 | 0.0042 | 0.65 | 0.55 | 0.6 | 0.61 | 6.32 | 0.005 | 0.0125 | 0.008 | 0.13 | 0.02 |
| 2437537 | 0.62 | 0.0027 | 1.37 | 0.6 | 0.0042 | 0.53 | 0.67 | 0.63 | 0.58 | 5.11 | 0.0031 | 0.0049 | 0.0035 | 0.49 | 0.153 |
| 2437538 | 0.65 | 0.0004 | 1.4 | 0.62 | 0.0007 | 0.71 | 0.53 | 0.61 | 0.64 | 6.07 | 0.0007 | 0.0006 | 0.0003 | 0.03 | 0.001 |
| 2437680 | 0.65 | 0.0004 | 1.3 | 0.61 | 0.0028 | 0.55 | 0.66 | 0.63 | 0.59 | 4.57 | 0.002 | 0.002 | 0.0015 | 0.08 | 0.037 |
| 2437775 | 0.62 | 0.0023 | 1.34 | 0.59 | 0.0095 | 0.48 | 0.71 | 0.63 | 0.57 | 4.27 | 0.0094 | 0.0051 | 0.0042 | 0.43 | 0.119 |
| 2439041 | 0.64 | 0.0009 | 1.27 | 0.62 | 0.0007 | 0.57 | 0.67 | 0.64 | 0.61 | 4.7 | 0.0005 | 0.0019 | 0.0014 | 0.18 | 0.071 |
| 2440149 | 0.6 | 0.0128 | 1.26 | 0.58 | 0.0197 | 0.53 | 0.63 | 0.6 | 0.57 | 4.89 | 0.0115 | 0.0112 | 0.0172 | 0.12 | 0.075 |
| 2440513 | 0.63 | 0.0015 | 1.27 | 0.61 | 0.0018 | 0.73 | 0.49 | 0.6 | 0.64 | 8 | 0.0021 | 0.0024 | 0.0023 | 0.06 | 0.005 |
| 2442702 | 0.63 | 0.0011 | 1.34 | 0.62 | 0.0011 | 0.53 | 0.7 | 0.65 | 0.59 | 5.39 | 0.0007 | 0.0022 | 0.002 | 0.03 | 0.006 |
| 2443917 | 0.63 | 0.0021 | 1.31 | 0.61 | 0.0028 | 0.53 | 0.68 | 0.64 | 0.59 | 4.6 | 0.0037 | 0.0035 | 0.0023 | 0.36 | 0.05 |
| 2444488 | 0.6 | 0.0142 | 1.3 | 0.6 | 0.0064 | 0.49 | 0.71 | 0.64 | 0.57 | 3.88 | 0.0038 | 0.0109 | 0.0118 | 0.27 | 0.055 |
| 2444489 | 0.62 | 0.0042 | 1.3 | 0.56 | 0.0681 | 0.41 | 0.72 | 0.6 | 0.54 | 4.28 | 0.0584 | 0.01 | 0.0111 | 0.29 | 0.075 |
| 2448289 | 0.64 | 0.0007 | 1.31 | 0.65 | 0 | 0.63 | 0.67 | 0.67 | 0.64 | 3.57 | 0.0001 | 0.0072 | 0.0037 | 0.12 | 0.026 |
| 2448313 | 0.63 | 0.0019 | 1.41 | 0.59 | 0.0095 | 0.5 | 0.68 | 0.62 | 0.57 | 4.02 | 0.0037 | 0.0022 | 0.0035 | 0.04 | 0.014 |
| 2450669 | 0.63 | 0.0019 | 1.41 | 0.6 | 0.0042 | 0.64 | 0.56 | 0.6 | 0.6 | 4.96 | 0.0033 | 0.0014 | 0.0012 | 0.02 | 0.003 |
| 2451314 | 0.61 | 0.0073 | 1.34 | 0.59 | 0.0095 | 0.54 | 0.64 | 0.61 | 0.58 | 4.6 | 0.0095 | 0.0029 | 0.0048 | 0.06 | 0.013 |
| 2452652 | 0.65 | 0.0002 | 1.4 | 0.65 | 0 | 0.69 | 0.6 | 0.64 | 0.66 | 6.12 | 0.0001 | 0.0002 | 0.0001 | 0.01 | 0.001 |
| 2452655 | 0.61 | 0.0053 | 1.41 | 0.61 | 0.0018 | 0.52 | 0.7 | 0.65 | 0.59 | 4.82 | 0.0005 | 0.002 | 0.0064 | 0.04 | 0.049 |
| 2457626 | 0.65 | 0.0003 | 1.28 | 0.64 | 0.0001 | 0.59 | 0.69 | 0.67 | 0.62 | 5.46 | 0 | 0.0012 | 0.0007 | 0.23 | 0.043 |
| 2458075 | 0.62 | 0.0033 | 1.46 | 0.59 | 0.0095 | 0.53 | 0.65 | 0.61 | 0.58 | 5.43 | 0.0052 | 0.0036 | 0.0043 | 0.05 | 0.016 |
| 2458381 | 0.62 | 0.0035 | 1.26 | 0.57 | 0.038 | 0.51 | 0.63 | 0.59 | 0.56 | 5.01 | 0.0397 | 0.003 | 0.0038 | 0.17 | 0.039 |
| 2458503 | 0.64 | 0.0005 | 1.3 | 0.62 | 0.0007 | 0.63 | 0.61 | 0.63 | 0.62 | 5.95 | 0.0007 | 0.0006 | 0.0005 | 0.04 | 0.004 |
| 2459655 | 0.63 | 0.0011 | 1.3 | 0.61 | 0.0028 | 0.56 | 0.65 | 0.63 | 0.59 | 6.21 | 0.0011 | 0.001 | 0.0011 | 0.08 | 0.013 |
| 2461827 | 0.66 | 0.0001 | 1.33 | 0.65 | 0 | 0.61 | 0.69 | 0.67 | 0.64 | 5.1 | 0 | 0.0004 | 0.0004 | 0.03 | 0.003 |
| 2461828 | 0.63 | 0.0011 | 1.26 | 0.6 | 0.0042 | 0.56 | 0.64 | 0.62 | 0.59 | 5.38 | 0.0039 | 0.0014 | 0.0012 | 0.06 | 0.005 |
| 2462178 | 0.61 | 0.0099 | 1.3 | 0.57 | 0.0513 | 0.5 | 0.63 | 0.59 | 0.55 | 4.63 | 0.0735 | 0.005 | 0.0071 | 0.09 | 0.018 |
| 2464496 | 0.65 | 0.0003 | 1.55 | 0.64 | 0.0001 | 0.58 | 0.69 | 0.66 | 0.62 | 5.59 | 0.0001 | 0.0001 | 0.0003 | 0.01 | 0.001 |
| 2464505 | 0.64 | 0.0006 | 1.28 | 0.61 | 0.0018 | 0.58 | 0.64 | 0.63 | 0.6 | 6.37 | 0.0019 | 0.0007 | 0.0007 | 0.04 | 0.005 |
| 2464514 | 0.63 | 0.002 | 1.28 | 0.62 | 0.0011 | 0.6 | 0.63 | 0.63 | 0.61 | 5.79 | 0.001 | 0.0023 | 0.0018 | 0.07 | 0.012 |
| 2464537 | 0.62 | 0.0042 | 1.34 | 0.65 | 0 | 0.62 | 0.68 | 0.67 | 0.64 | 5.63 | 0 | 0.003 | 0.0032 | 0.15 | 0.011 |
| 2468935 | 0.6 | 0.0107 | 1.27 | 0.58 | 0.0197 | 0.56 | 0.6 | 0.59 | 0.57 | 4.61 | 0.0509 | 0.0299 | 0.01 | 0.29 | 0.034 |
| 2469568 | 0.64 | 0.0006 | 1.29 | 0.61 | 0.0028 | 0.49 | 0.73 | 0.65 | 0.58 | 4.39 | 0.0014 | 0.0039 | 0.0036 | 0.09 | 0.031 |
| 2473241 | 0.67 | 0.0001 | 1.25 | 0.63 | 0.0002 | 0.56 | 0.7 | 0.66 | 0.61 | 4.78 | 0.0003 | 0.0002 | 0.0002 | 0.07 | 0.013 |
| 2473624 | 0.64 | 0.0009 | 1.57 | 0.63 | 0.0002 | 0.62 | 0.64 | 0.64 | 0.62 | 5.03 | 0.0003 | 0.0011 | 0.0007 | 0.01 | 0.001 |
| 2474147 | 0.61 | 0.0063 | 1.28 | 0.6 | 0.0064 | 0.67 | 0.52 | 0.59 | 0.61 | 6.87 | 0.0067 | 0.0054 | 0.0067 | 0.08 | 0.042 |
| 2474700 | 0.65 | 0.0003 | 1.34 | 0.64 | 0.0001 | 0.69 | 0.59 | 0.64 | 0.65 | 5.15 | 0.0001 | 0.0006 | 0.0004 | 0.05 | 0.005 |
| 2474770 | 0.64 | 0.0006 | 1.29 | 0.64 | 0.0001 | 0.64 | 0.64 | 0.65 | 0.64 | 6.1 | 0.0001 | 0.0011 | 0.0005 | 0.02 | 0.002 |
| 2475647 | 0.61 | 0.0101 | 1.28 | 0.58 | 0.0276 | 0.48 | 0.68 | 0.61 | 0.56 | 5.08 | 0.0463 | 0.0278 | 0.0158 | 0.29 | 0.094 |
| 2476316 | 0.66 | 0.0001 | 1.25 | 0.64 | 0.0001 | 0.57 | 0.7 | 0.67 | 0.62 | 4.91 | 0.0001 | 0.0004 | 0.0004 | 0.09 | 0.007 |
| 2476408 | 0.63 | 0.0022 | 1.53 | 0.63 | 0.0004 | 0.61 | 0.64 | 0.64 | 0.62 | 4.89 | 0.0003 | 0.0013 | 0.0017 | 0.02 | 0.006 |
| 2477341 | 0.6 | 0.0128 | 1.26 | 0.6 | 0.0042 | 0.61 | 0.59 | 0.61 | 0.6 | 5.26 | 0.0028 | 0.0224 | 0.0138 | 0.3 | 0.047 |
| 2478839 | 0.62 | 0.005 | 1.25 | 0.62 | 0.0007 | 0.61 | 0.63 | 0.63 | 0.61 | 6.44 | 0.001 | 0.008 | 0.0045 | 0.09 | 0.006 |
| 2479729 | 0.64 | 0.0004 | 1.3 | 0.61 | 0.0028 | 0.66 | 0.55 | 0.6 | 0.61 | 5.19 | 0.0021 | 0.0006 | 0.0005 | 0.02 | 0.002 |
| 2480989 | 0.63 | 0.0011 | 1.31 | 0.57 | 0.038 | 0.45 | 0.7 | 0.61 | 0.55 | 4.89 | 0.0197 | 0.0019 | 0.0016 | 0.03 | 0.003 |
| 2481178 | 0.64 | 0.0007 | 1.44 | 0.63 | 0.0002 | 0.6 | 0.66 | 0.65 | 0.62 | 4.99 | 0.0003 | 0.001 | 0.0008 | 0.02 | 0.002 |
| 2482593 | 0.63 | 0.0012 | 1.33 | 0.61 | 0.0018 | 0.56 | 0.66 | 0.63 | 0.6 | 5.41 | 0.0011 | 0.0023 | 0.002 | 0.03 | 0.005 |
| 2482624 | 0.63 | 0.0014 | 1.25 | 0.63 | 0.0002 | 0.72 | 0.54 | 0.62 | 0.65 | 6.89 | 0.0003 | 0.0019 | 0.0015 | 0.03 | 0.003 |
| 2483047 | 0.61 | 0.0058 | 1.34 | 0.59 | 0.0095 | 0.57 | 0.61 | 0.6 | 0.58 | 4.92 | 0.0052 | 0.01 | 0.0104 | 0.06 | 0.013 |
| 2484558 | 0.65 | 0.0003 | 1.42 | 0.64 | 0.0001 | 0.49 | 0.8 | 0.71 | 0.6 | 4.17 | 0 | 0.0002 | 0.0003 | 0.03 | 0.002 |
| 2484807 | 0.62 | 0.0035 | 1.25 | 0.6 | 0.0042 | 0.71 | 0.49 | 0.59 | 0.62 | 6.47 | 0.0057 | 0.0048 | 0.0023 | 0.01 | 0.001 |
| 2485214 | 0.6 | 0.0136 | 1.3 | 0.58 | 0.0276 | 0.54 | 0.61 | 0.59 | 0.57 | 5.47 | 0.0351 | 0.0224 | 0.0135 | 0.08 | 0.009 |
| 2485276 | 0.61 | 0.0085 | 1.29 | 0.59 | 0.0138 | 0.54 | 0.63 | 0.6 | 0.57 | 4.36 | 0.0166 | 0.0417 | 0.0294 | 0.31 | 0.1 |
| 2487216 | 0.61 | 0.0063 | 1.39 | 0.58 | 0.0276 | 0.51 | 0.64 | 0.6 | 0.56 | 5.37 | 0.0365 | 0.0035 | 0.0042 | 0.13 | 0.024 |
| 2487645 | 0.63 | 0.0015 | 1.27 | 0.63 | 0.0004 | 0.57 | 0.68 | 0.65 | 0.61 | 5.9 | 0.0005 | 0.0034 | 0.0019 | 0.09 | 0.011 |
| 2488150 | 0.65 | 0.0002 | 1.27 | 0.62 | 0.0011 | 0.61 | 0.62 | 0.63 | 0.61 | 5.02 | 0.0012 | 0.0001 | 0.0002 | 0.02 | 0.007 |
| 2489030 | 0.62 | 0.0043 | 1.28 | 0.59 | 0.0138 | 0.53 | 0.64 | 0.61 | 0.57 | 5.84 | 0.0133 | 0.0086 | 0.0042 | 0.29 | 0.035 |
| 2489435 | 0.63 | 0.0019 | 1.25 | 0.63 | 0.0002 | 0.65 | 0.61 | 0.63 | 0.63 | 6.02 | 0.0002 | 0.0021 | 0.0016 | 0.01 | 0.003 |
| 2491645 | 0.64 | 0.0006 | 1.27 | 0.62 | 0.0007 | 0.62 | 0.62 | 0.63 | 0.62 | 6.38 | 0.0005 | 0.0009 | 0.0007 | 0.04 | 0.007 |
| 2492101 | 0.63 | 0.0013 | 1.25 | 0.6 | 0.0064 | 0.5 | 0.69 | 0.63 | 0.58 | 3.78 | 0.0078 | 0.0014 | 0.003 | 0.04 | 0.01 |
| 2492833 | 0.61 | 0.0066 | 1.34 | 0.59 | 0.0138 | 0.55 | 0.62 | 0.6 | 0.58 | 5.88 | 0.0047 | 0.0018 | 0.0054 | 0.04 | 0.089 |
| 2495140 | 0.63 | 0.0015 | 1.43 | 0.62 | 0.0011 | 0.65 | 0.58 | 0.62 | 0.62 | 4.83 | 0.0013 | 0.002 | 0.0016 | 0.08 | 0.006 |
| 2498988 | 0.61 | 0.006 | 1.3 | 0.58 | 0.0197 | 0.48 | 0.69 | 0.62 | 0.56 | 4.48 | 0.0246 | 0.0105 | 0.011 | 0.13 | 0.028 |
| 2507241 | 0.66 | 0.0001 | 1.4 | 0.61 | 0.0028 | 0.51 | 0.7 | 0.64 | 0.58 | 5.19 | 0.0019 | 0.0004 | 0.0004 | 0.05 | 0.005 |
| 2507514 | 0.62 | 0.0031 | 1.25 | 0.58 | 0.0197 | 0.53 | 0.63 | 0.6 | 0.57 | 6.15 | 0.0392 | 0.0044 | 0.0034 | 0.02 | 0.003 |
| 2507533 | 0.64 | 0.0005 | 1.25 | 0.64 | 0.0001 | 0.64 | 0.64 | 0.65 | 0.64 | 6.62 | 0.0001 | 0.0005 | 0.0004 | 0.04 | 0.003 |
| 2512351 | 0.62 | 0.0024 | 1.29 | 0.62 | 0.0007 | 0.56 | 0.68 | 0.65 | 0.6 | 4.68 | 0.0009 | 0.0053 | 0.0049 | 0.08 | 0.02 |
| 2512363 | 0.62 | 0.0044 | 1.3 | 0.57 | 0.038 | 0.5 | 0.64 | 0.59 | 0.56 | 3.74 | 0.0309 | 0.0093 | 0.0079 | 0.05 | 0.01 |
| 2512959 | 0.63 | 0.0013 | 1.26 | 0.6 | 0.0064 | 0.56 | 0.63 | 0.61 | 0.58 | 3.93 | 0.0045 | 0.0039 | 0.0033 | 0.04 | 0.013 |
| 2515085 | 0.63 | 0.0017 | 1.27 | 0.63 | 0.0004 | 0.63 | 0.62 | 0.63 | 0.62 | 7.13 | 0.0003 | 0.001 | 0.0016 | 0.03 | 0.017 |
| 2517330 | 0.64 | 0.0009 | 1.31 | 0.6 | 0.0042 | 0.45 | 0.77 | 0.66 | 0.57 | 4.33 | 0.0023 | 0.0035 | 0.0028 | 0.13 | 0.025 |
| 2518193 | 0.63 | 0.0013 | 1.27 | 0.63 | 0.0004 | 0.71 | 0.54 | 0.62 | 0.65 | 5.44 | 0.0005 | 0.0029 | 0.0024 | 0.15 | 0.008 |
| 2518707 | 0.63 | 0.0018 | 1.29 | 0.59 | 0.0138 | 0.54 | 0.63 | 0.6 | 0.57 | 5.24 | 0.0072 | 0.0009 | 0.0024 | 0.03 | 0.034 |
| 2525095 | 0.65 | 0.0003 | 1.29 | 0.62 | 0.0007 | 0.58 | 0.66 | 0.64 | 0.61 | 5.56 | 0.001 | 0.0014 | 0.001 | 0.09 | 0.004 |
| 2527206 | 0.63 | 0.0016 | 1.29 | 0.6 | 0.0042 | 0.69 | 0.51 | 0.59 | 0.62 | 8.09 | 0.0037 | 0.0025 | 0.0023 | 0.09 | 0.008 |
| 2527635 | 0.62 | 0.0033 | 1.25 | 0.58 | 0.0197 | 0.52 | 0.64 | 0.6 | 0.57 | 4.94 | 0.0205 | 0.0046 | 0.0041 | 0.06 | 0.007 |
| 2532844 | 0.62 | 0.0051 | 1.26 | 0.59 | 0.0138 | 0.5 | 0.68 | 0.62 | 0.57 | 4.66 | 0.017 | 0.0122 | 0.009 | 0.26 | 0.059 |
| 2534447 | 0.63 | 0.0014 | 1.29 | 0.62 | 0.0011 | 0.51 | 0.72 | 0.66 | 0.59 | 5.77 | 0.001 | 0.0027 | 0.0018 | 0.06 | 0.009 |
| 2537112 | 0.64 | 0.0007 | 1.35 | 0.63 | 0.0002 | 0.65 | 0.61 | 0.63 | 0.63 | 6.32 | 0.0002 | 0.0006 | 0.0009 | 0.03 | 0.011 |
| 2539775 | 0.63 | 0.0011 | 1.33 | 0.61 | 0.0028 | 0.5 | 0.72 | 0.65 | 0.58 | 4.61 | 0.0023 | 0.0024 | 0.0016 | 0.09 | 0.011 |
| 2540221 | 0.62 | 0.0038 | 1.32 | 0.63 | 0.0004 | 0.67 | 0.58 | 0.62 | 0.63 | 6.24 | 0.0008 | 0.0033 | 0.0023 | 0.05 | 0.007 |
| 2544180 | 0.62 | 0.0041 | 1.27 | 0.62 | 0.0007 | 0.62 | 0.62 | 0.63 | 0.62 | 6.55 | 0.0006 | 0.0047 | 0.0028 | 0.04 | 0.003 |
| 2544185 | 0.6 | 0.0107 | 1.27 | 0.55 | 0.1438 | 0.35 | 0.76 | 0.59 | 0.53 | 4.72 | 0.0545 | 0.0234 | 0.0369 | 0.14 | 0.11 |
| 2544295 | 0.61 | 0.0059 | 1.43 | 0.59 | 0.0138 | 0.54 | 0.63 | 0.6 | 0.57 | 4.66 | 0.0201 | 0.011 | 0.0081 | 0.39 | 0.11 |
| 2544306 | 0.62 | 0.0045 | 1.25 | 0.59 | 0.0138 | 0.43 | 0.76 | 0.64 | 0.56 | 5.45 | 0.015 | 0.014 | 0.0103 | 0.49 | 0.177 |
| 2546279 | 0.62 | 0.0029 | 1.49 | 0.62 | 0.0007 | 0.64 | 0.6 | 0.63 | 0.62 | 5.03 | 0.0014 | 0.0063 | 0.003 | 0.11 | 0.009 |
| 2549323 | 0.62 | 0.0032 | 1.34 | 0.59 | 0.0095 | 0.52 | 0.66 | 0.62 | 0.58 | 4.74 | 0.0102 | 0.0024 | 0.0049 | 0.03 | 0.015 |
| 2550840 | 0.62 | 0.0035 | 1.27 | 0.63 | 0.0004 | 0.54 | 0.71 | 0.66 | 0.6 | 4.91 | 0.0001 | 0.0043 | 0.006 | 0.1 | 0.022 |
| 2550842 | 0.62 | 0.0034 | 1.33 | 0.59 | 0.0138 | 0.5 | 0.67 | 0.61 | 0.57 | 3.67 | 0.0178 | 0.0125 | 0.0106 | 0.09 | 0.023 |
| 2550964 | 0.67 | 0 | 1.27 | 0.64 | 0.0001 | 0.61 | 0.67 | 0.66 | 0.63 | 5.74 | 0.0001 | 0.0001 | 0.0001 | 0.02 | 0.001 |
| 2552021 | 0.63 | 0.0017 | 1.5 | 0.61 | 0.0018 | 0.53 | 0.69 | 0.64 | 0.59 | 4.86 | 0.0011 | 0.0028 | 0.002 | 0.06 | 0.006 |
| 2553312 | 0.63 | 0.002 | 1.27 | 0.63 | 0.0004 | 0.63 | 0.62 | 0.63 | 0.62 | 5.72 | 0.0004 | 0.0023 | 0.0021 | 0.12 | 0.015 |
| 2553583 | 0.63 | 0.0012 | 1.27 | 0.6 | 0.0042 | 0.71 | 0.49 | 0.59 | 0.62 | 7.2 | 0.0063 | 0.0019 | 0.0014 | 0.05 | 0.004 |
| 2553585 | 0.63 | 0.0013 | 1.71 | 0.61 | 0.0018 | 0.52 | 0.7 | 0.65 | 0.59 | 5.48 | 0.0006 | 0.0006 | 0.001 | 0.04 | 0.011 |
| 2553595 | 0.62 | 0.0031 | 1.38 | 0.6 | 0.0042 | 0.57 | 0.63 | 0.62 | 0.59 | 5.43 | 0.0024 | 0.0023 | 0.0029 | 0.07 | 0.015 |
| 2553907 | 0.63 | 0.0012 | 1.31 | 0.59 | 0.0095 | 0.47 | 0.72 | 0.64 | 0.57 | 4.28 | 0.0043 | 0.0028 | 0.0039 | 0.19 | 0.052 |
| 2554001 | 0.63 | 0.0018 | 1.42 | 0.6 | 0.0042 | 0.5 | 0.71 | 0.64 | 0.58 | 3.88 | 0.0009 | 0.0015 | 0.0025 | 0.06 | 0.037 |
| 2555279 | 0.62 | 0.004 | 1.36 | 0.6 | 0.0064 | 0.56 | 0.63 | 0.61 | 0.58 | 5.6 | 0.0113 | 0.0095 | 0.0048 | 0.2 | 0.024 |
| 2555281 | 0.64 | 0.0006 | 1.29 | 0.58 | 0.0197 | 0.45 | 0.72 | 0.63 | 0.56 | 4.35 | 0.0033 | 0.0021 | 0.0029 | 0.01 | 0.007 |
| 2555288 | 0.62 | 0.0028 | 1.29 | 0.6 | 0.0064 | 0.5 | 0.69 | 0.63 | 0.58 | 4.4 | 0.0026 | 0.0029 | 0.0052 | 0.11 | 0.052 |
| 2555330 | 0.65 | 0.0004 | 1.28 | 0.6 | 0.0042 | 0.45 | 0.77 | 0.66 | 0.57 | 4.24 | 0.0025 | 0.0011 | 0.0012 | 0.21 | 0.039 |
| 2555409 | 0.62 | 0.0046 | 1.31 | 0.61 | 0.0028 | 0.52 | 0.69 | 0.64 | 0.59 | 4.36 | 0.0011 | 0.0022 | 0.0053 | 0.05 | 0.013 |
| 2555411 | 0.64 | 0.0008 | 1.46 | 0.62 | 0.0007 | 0.62 | 0.62 | 0.63 | 0.62 | 5.58 | 0.0008 | 0.0008 | 0.0007 | 0.15 | 0.016 |
| 2555416 | 0.64 | 0.0004 | 1.27 | 0.62 | 0.0007 | 0.53 | 0.71 | 0.66 | 0.6 | 5.26 | 0.0006 | 0.0007 | 0.0008 | 0.2 | 0.039 |
| 2555507 | 0.61 | 0.0068 | 1.25 | 0.53 | 0.2618 | 0.3 | 0.78 | 0.58 | 0.52 | 4.25 | 0.2445 | 0.0242 | 0.0249 | 0.19 | 0.039 |
| 2555522 | 0.62 | 0.0026 | 1.26 | 0.62 | 0.0007 | 0.69 | 0.55 | 0.61 | 0.64 | 5.18 | 0.0018 | 0.0044 | 0.004 | 0.07 | 0.03 |
| 2556229 | 0.62 | 0.0024 | 1.39 | 0.6 | 0.0064 | 0.52 | 0.67 | 0.62 | 0.58 | 4.22 | 0.0064 | 0.0048 | 0.0049 | 0.17 | 0.037 |
| 2558208 | 0.62 | 0.003 | 1.26 | 0.6 | 0.0064 | 0.5 | 0.7 | 0.63 | 0.58 | 4.27 | 0.0027 | 0.0032 | 0.0055 | 0.01 | 0.021 |
| 2558313 | 0.65 | 0.0003 | 1.28 | 0.63 | 0.0004 | 0.55 | 0.7 | 0.66 | 0.61 | 5.59 | 0.0003 | 0.0003 | 0.0003 | 0.01 | 0.002 |
| 2558519 | 0.63 | 0.002 | 1.35 | 0.64 | 0.0001 | 0.61 | 0.66 | 0.65 | 0.63 | 5.68 | 0.0001 | 0.0015 | 0.0015 | 0.21 | 0.018 |
| 2559293 | 0.65 | 0.0004 | 1.27 | 0.63 | 0.0004 | 0.52 | 0.73 | 0.67 | 0.6 | 5.19 | 0.0002 | 0.0004 | 0.0008 | 0.03 | 0.02 |
| 2562334 | 0.65 | 0.0004 | 1.3 | 0.65 | 0 | 0.66 | 0.63 | 0.65 | 0.65 | 6.84 | 0 | 0.0003 | 0.0003 | 0.02 | 0.004 |
| 2565264 | 0.64 | 0.0006 | 1.33 | 0.64 | 0.0001 | 0.68 | 0.6 | 0.64 | 0.65 | 7.48 | 0.0001 | 0.0005 | 0.0003 | 0.02 | 0.001 |
| 2575142 | 0.6 | 0.0129 | 1.32 | 0.58 | 0.0197 | 0.5 | 0.66 | 0.61 | 0.57 | 4.65 | 0.0195 | 0.0287 | 0.019 | 0.06 | 0.016 |
| 2577959 | 0.64 | 0.0009 | 1.27 | 0.59 | 0.0138 | 0.48 | 0.7 | 0.62 | 0.57 | 3.97 | 0.0115 | 0.0024 | 0.0026 | 0.06 | 0.022 |
| 2581588 | 0.61 | 0.0061 | 1.28 | 0.57 | 0.038 | 0.54 | 0.6 | 0.59 | 0.56 | 4.26 | 0.0358 | 0.0105 | 0.0101 | 0.06 | 0.027 |
| 2583054 | 0.64 | 0.0009 | 1.28 | 0.62 | 0.0011 | 0.65 | 0.58 | 0.62 | 0.62 | 6.29 | 0.0018 | 0.0014 | 0.0009 | 0.03 | 0.003 |
| 2583056 | 0.65 | 0.0003 | 1.35 | 0.65 | 0 | 0.62 | 0.67 | 0.66 | 0.63 | 4.37 | 0.0001 | 0.0016 | 0.0006 | 0.12 | 0.009 |
| 2583249 | 0.61 | 0.0102 | 1.31 | 0.59 | 0.0138 | 0.5 | 0.67 | 0.61 | 0.57 | 4.69 | 0.0143 | 0.009 | 0.0093 | 0.06 | 0.017 |
| 2583609 | 0.6 | 0.012 | 1.32 | 0.59 | 0.0138 | 0.54 | 0.63 | 0.6 | 0.57 | 5.38 | 0.0137 | 0.0112 | 0.0102 | 0.1 | 0.025 |
| 2584845 | 0.64 | 0.001 | 1.3 | 0.63 | 0.0004 | 0.63 | 0.62 | 0.63 | 0.62 | 6 | 0.0002 | 0.0006 | 0.0013 | 0.01 | 0.004 |
| 2588838 | 0.61 | 0.0058 | 1.27 | 0.57 | 0.038 | 0.51 | 0.63 | 0.59 | 0.56 | 5.09 | 0.0173 | 0.0064 | 0.0114 | 0.18 | 0.11 |
| 2590750 | 0.64 | 0.0008 | 1.42 | 0.64 | 0.0001 | 0.59 | 0.68 | 0.66 | 0.62 | 5.38 | 0 | 0.0011 | 0.0014 | 0.02 | 0.014 |
| 2590802 | 0.62 | 0.0046 | 1.51 | 0.65 | 0 | 0.6 | 0.69 | 0.67 | 0.63 | 7 | 0 | 0.0023 | 0.0027 | 0.07 | 0.011 |
| 2591635 | 0.6 | 0.0145 | 1.25 | 0.6 | 0.0042 | 0.6 | 0.6 | 0.61 | 0.6 | 6.9 | 0.0046 | 0.0107 | 0.017 | 0.18 | 0.053 |
| 2591638 | 0.61 | 0.0052 | 1.36 | 0.62 | 0.0011 | 0.53 | 0.7 | 0.65 | 0.59 | 6.82 | 0.0005 | 0.0027 | 0.0054 | 0.09 | 0.016 |
| 2593447 | 0.61 | 0.0059 | 1.32 | 0.58 | 0.0197 | 0.5 | 0.67 | 0.61 | 0.56 | 4.62 | 0.0131 | 0.0105 | 0.0139 | 0.11 | 0.031 |
| 2593692 | 0.61 | 0.0064 | 1.35 | 0.58 | 0.0197 | 0.54 | 0.62 | 0.6 | 0.57 | 5.2 | 0.0159 | 0.006 | 0.0044 | 0.02 | 0.002 |
| 2593714 | 0.61 | 0.0093 | 1.31 | 0.59 | 0.0095 | 0.51 | 0.67 | 0.62 | 0.57 | 4.97 | 0.0075 | 0.0075 | 0.0121 | 0.11 | 0.03 |
| 2593758 | 0.61 | 0.0063 | 1.53 | 0.63 | 0.0004 | 0.66 | 0.59 | 0.63 | 0.63 | 4.96 | 0.0002 | 0.0023 | 0.004 | 0.07 | 0.029 |
| 2594506 | 0.61 | 0.0077 | 1.31 | 0.61 | 0.0018 | 0.51 | 0.71 | 0.65 | 0.59 | 4.28 | 0.0016 | 0.0292 | 0.0191 | 0.33 | 0.084 |
| 2596691 | 0.63 | 0.0017 | 1.28 | 0.63 | 0.0004 | 0.69 | 0.56 | 0.62 | 0.64 | 4.81 | 0.0007 | 0.0048 | 0.0028 | 0.03 | 0.005 |
| 2597013 | 0.64 | 0.0009 | 1.28 | 0.61 | 0.0028 | 0.56 | 0.65 | 0.63 | 0.59 | 4.94 | 0.0039 | 0.0014 | 0.001 | 0.02 | 0.003 |
| 2598267 | 0.6 | 0.0108 | 1.25 | 0.6 | 0.0042 | 0.61 | 0.59 | 0.61 | 0.6 | 6.26 | 0.0053 | 0.0044 | 0.0126 | 0.07 | 0.026 |
| 2598289 | 0.63 | 0.002 | 1.29 | 0.58 | 0.0197 | 0.47 | 0.7 | 0.62 | 0.56 | 5.73 | 0.0298 | 0.0015 | 0.0035 | 0.02 | 0.004 |
| 2598781 | 0.63 | 0.0021 | 1.26 | 0.64 | 0.0001 | 0.68 | 0.59 | 0.63 | 0.64 | 8.61 | 0.0002 | 0.0027 | 0.0024 | 0.09 | 0.007 |
| 2599342 | 0.61 | 0.0055 | 1.4 | 0.6 | 0.0042 | 0.51 | 0.69 | 0.63 | 0.58 | 5.58 | 0.005 | 0.0074 | 0.0064 | 0.05 | 0.01 |
| 2599903 | 0.61 | 0.0085 | 1.37 | 0.59 | 0.0138 | 0.51 | 0.66 | 0.61 | 0.57 | 4.45 | 0.0279 | 0.0131 | 0.0066 | 0.17 | 0.017 |
| 2606647 | 0.6 | 0.0122 | 1.25 | 0.58 | 0.0276 | 0.61 | 0.54 | 0.58 | 0.58 | 6.59 | 0.0576 | 0.0127 | 0.0076 | 0.18 | 0.019 |
| 2607152 | 0.62 | 0.0037 | 1.3 | 0.61 | 0.0018 | 0.58 | 0.64 | 0.63 | 0.6 | 6.07 | 0.0039 | 0.0044 | 0.004 | 0.14 | 0.024 |
| 2607374 | 0.6 | 0.012 | 1.34 | 0.56 | 0.0889 | 0.42 | 0.7 | 0.59 | 0.54 | 5.08 | 0.0849 | 0.026 | 0.0232 | 0.06 | 0.042 |
| 2609635 | 0.62 | 0.003 | 1.3 | 0.61 | 0.0028 | 0.63 | 0.58 | 0.61 | 0.61 | 4.58 | 0.003 | 0.0027 | 0.0032 | 0.08 | 0.019 |
| 2609657 | 0.63 | 0.0021 | 1.36 | 0.63 | 0.0004 | 0.53 | 0.72 | 0.67 | 0.6 | 5.28 | 0.0002 | 0.0012 | 0.0018 | 0.06 | 0.023 |
| 2609668 | 0.61 | 0.0055 | 1.25 | 0.61 | 0.0028 | 0.62 | 0.59 | 0.61 | 0.6 | 5.62 | 0.0024 | 0.0041 | 0.0082 | 0.12 | 0.038 |
| 2610353 | 0.6 | 0.0138 | 1.33 | 0.6 | 0.0064 | 0.6 | 0.59 | 0.6 | 0.59 | 5.54 | 0.0054 | 0.0091 | 0.012 | 0.2 | 0.055 |
| 2611083 | 0.62 | 0.0037 | 1.26 | 0.6 | 0.0064 | 0.42 | 0.79 | 0.67 | 0.57 | 5.68 | 0.0019 | 0.002 | 0.0032 | 0.25 | 0.04 |
| 2614100 | 0.61 | 0.0051 | 1.33 | 0.62 | 0.0007 | 0.53 | 0.71 | 0.66 | 0.6 | 5.7 | 0.0002 | 0.0025 | 0.0052 | 0.02 | 0.01 |
| 2614135 | 0.65 | 0.0003 | 1.33 | 0.61 | 0.0028 | 0.53 | 0.68 | 0.64 | 0.59 | 5.24 | 0.0038 | 0.0006 | 0.0006 | 0.05 | 0.002 |
| 2614137 | 0.61 | 0.0082 | 1.32 | 0.6 | 0.0064 | 0.53 | 0.66 | 0.62 | 0.58 | 5.83 | 0.0051 | 0.007 | 0.006 | 0.14 | 0.036 |
| 2615656 | 0.63 | 0.0013 | 1.33 | 0.6 | 0.0064 | 0.53 | 0.66 | 0.62 | 0.58 | 5.53 | 0.0034 | 0.0032 | 0.003 | 0.07 | 0.024 |
| 2616071 | 0.63 | 0.0019 | 1.46 | 0.62 | 0.0011 | 0.53 | 0.7 | 0.65 | 0.59 | 4.56 | 0.0005 | 0.0018 | 0.0024 | 0.05 | 0.018 |
| 2616248 | 0.62 | 0.0026 | 1.32 | 0.6 | 0.0064 | 0.52 | 0.67 | 0.62 | 0.58 | 4.93 | 0.0108 | 0.0114 | 0.0044 | 0.48 | 0.04 |
| 2617578 | 0.61 | 0.0057 | 1.33 | 0.59 | 0.0095 | 0.68 | 0.5 | 0.58 | 0.6 | 5.79 | 0.018 | 0.011 | 0.0056 | 0.16 | 0.012 |
| 2618656 | 0.63 | 0.0014 | 1.36 | 0.61 | 0.0018 | 0.68 | 0.54 | 0.61 | 0.62 | 5.73 | 0.0025 | 0.0022 | 0.0013 | 0.02 | 0.002 |
| 2619365 | 0.63 | 0.0011 | 1.34 | 0.62 | 0.0011 | 0.58 | 0.65 | 0.63 | 0.6 | 4.86 | 0.0012 | 0.0023 | 0.0025 | 0.13 | 0.03 |
| 2625632 | 0.65 | 0.0004 | 1.3 | 0.6 | 0.0042 | 0.59 | 0.61 | 0.61 | 0.59 | 4.15 | 0.0049 | 0.0009 | 0.0006 | 0.04 | 0.004 |
| 2625643 | 0.65 | 0.0002 | 1.33 | 0.62 | 0.0007 | 0.58 | 0.66 | 0.64 | 0.61 | 5.84 | 0.0008 | 0.0004 | 0.0003 | 0.06 | 0.005 |
| 2625893 | 0.64 | 0.0007 | 1.37 | 0.58 | 0.0276 | 0.44 | 0.72 | 0.62 | 0.55 | 3.76 | 0.0209 | 0.0035 | 0.003 | 0.02 | 0.01 |
| 2627951 | 0.67 | 0 | 1.33 | 0.63 | 0.0002 | 0.5 | 0.77 | 0.69 | 0.6 | 4.23 | 0.0001 | 0.0002 | 0.0002 | 0.01 | 0.001 |
| 2633534 | 0.63 | 0.0014 | 1.25 | 0.57 | 0.038 | 0.43 | 0.72 | 0.61 | 0.55 | 4.54 | 0.0237 | 0.0035 | 0.003 | 0.01 | 0.004 |
| 2633651 | 0.63 | 0.0021 | 1.27 | 0.62 | 0.0011 | 0.55 | 0.68 | 0.64 | 0.6 | 5.83 | 0.0011 | 0.0034 | 0.0027 | 0.03 | 0.004 |
| 2638377 | 0.63 | 0.0018 | 1.26 | 0.58 | 0.0276 | 0.48 | 0.68 | 0.61 | 0.56 | 4.37 | 0.015 | 0.0052 | 0.0046 | 0.05 | 0.014 |
| 2638451 | 0.62 | 0.0048 | 1.29 | 0.62 | 0.0011 | 0.62 | 0.61 | 0.62 | 0.61 | 7.12 | 0.0011 | 0.0033 | 0.0028 | 0.1 | 0.013 |
| 2638452 | 0.61 | 0.0067 | 1.32 | 0.6 | 0.0064 | 0.67 | 0.52 | 0.59 | 0.61 | 6.87 | 0.0087 | 0.005 | 0.0036 | 0.03 | 0.006 |
| 2641575 | 0.61 | 0.0097 | 1.28 | 0.6 | 0.0064 | 0.56 | 0.63 | 0.61 | 0.58 | 5.25 | 0.0063 | 0.0196 | 0.0121 | 0.16 | 0.047 |
| 2643579 | 0.62 | 0.0045 | 1.28 | 0.6 | 0.0064 | 0.53 | 0.66 | 0.62 | 0.58 | 4.81 | 0.0052 | 0.0038 | 0.0044 | 0.23 | 0.093 |
| 2645294 | 0.65 | 0.0002 | 1.46 | 0.66 | 0 | 0.6 | 0.71 | 0.69 | 0.64 | 5.72 | 0 | 0.0001 | 0.0001 | 0.03 | 0.003 |
| 2646066 | 0.61 | 0.0088 | 1.25 | 0.62 | 0.0007 | 0.65 | 0.59 | 0.62 | 0.62 | 5.06 | 0.0009 | 0.0173 | 0.0143 | 0.08 | 0.023 |
| 2646085 | 0.64 | 0.0007 | 1.51 | 0.63 | 0.0004 | 0.67 | 0.58 | 0.62 | 0.63 | 5.34 | 0.0005 | 0.0005 | 0.0005 | 0.05 | 0.004 |
| 2647361 | 0.63 | 0.0011 | 1.36 | 0.63 | 0.0004 | 0.54 | 0.71 | 0.66 | 0.6 | 5.39 | 0.0003 | 0.002 | 0.0016 | 0.29 | 0.087 |
| 2647678 | 0.65 | 0.0003 | 1.3 | 0.64 | 0.0001 | 0.54 | 0.73 | 0.68 | 0.61 | 4.48 | 0 | 0.0003 | 0.0007 | 0 | 0.003 |
| 2647775 | 0.6 | 0.0125 | 1.32 | 0.6 | 0.0042 | 0.52 | 0.68 | 0.63 | 0.58 | 5.37 | 0.0011 | 0.0055 | 0.0089 | 0.4 | 0.074 |
| 2647779 | 0.62 | 0.0042 | 1.3 | 0.6 | 0.0042 | 0.55 | 0.65 | 0.62 | 0.59 | 4.39 | 0.0024 | 0.0018 | 0.0045 | 0 | 0.002 |
| 2648202 | 0.62 | 0.0028 | 1.27 | 0.63 | 0.0004 | 0.65 | 0.6 | 0.63 | 0.63 | 4.61 | 0.0004 | 0.0084 | 0.0074 | 0.15 | 0.054 |
| 2651521 | 0.66 | 0.0001 | 1.31 | 0.64 | 0.0001 | 0.56 | 0.71 | 0.67 | 0.61 | 4.58 | 0 | 0.0001 | 0.0002 | 0.06 | 0.005 |
| 2652021 | 0.65 | 0.0004 | 1.47 | 0.63 | 0.0004 | 0.54 | 0.71 | 0.66 | 0.6 | 5.18 | 0.0004 | 0.0004 | 0.0006 | 0.06 | 0.008 |
| 2654335 | 0.66 | 0.0002 | 1.41 | 0.61 | 0.0018 | 0.51 | 0.71 | 0.65 | 0.59 | 5.41 | 0.0005 | 0.0002 | 0.0002 | 0.04 | 0.003 |
| 2654874 | 0.64 | 0.0008 | 1.3 | 0.65 | 0 | 0.63 | 0.67 | 0.67 | 0.64 | 5.77 | 0 | 0.0021 | 0.001 | 0.11 | 0.012 |
| 2654878 | 0.61 | 0.0102 | 1.25 | 0.55 | 0.1438 | 0.42 | 0.68 | 0.58 | 0.53 | 4.05 | 0.1136 | 0.0115 | 0.0178 | 0.12 | 0.042 |
| 2655475 | 0.66 | 0.0001 | 1.34 | 0.63 | 0.0004 | 0.59 | 0.66 | 0.65 | 0.61 | 5.11 | 0.0002 | 0.0001 | 0.0001 | 0.02 | 0.002 |
| 2655509 | 0.64 | 0.0008 | 1.27 | 0.61 | 0.0028 | 0.63 | 0.58 | 0.61 | 0.61 | 8.77 | 0.0046 | 0.0008 | 0.0008 | 0.02 | 0.002 |
| 2655678 | 0.61 | 0.0062 | 1.26 | 0.61 | 0.0028 | 0.59 | 0.62 | 0.62 | 0.6 | 5.74 | 0.0011 | 0.003 | 0.0057 | 0.05 | 0.018 |
| 2656791 | 0.64 | 0.001 | 1.25 | 0.61 | 0.0018 | 0.65 | 0.57 | 0.61 | 0.62 | 7.16 | 0.0018 | 0.0013 | 0.0009 | 0.06 | 0.005 |
| 2658354 | 0.64 | 0.0009 | 1.26 | 0.66 | 0 | 0.63 | 0.68 | 0.67 | 0.64 | 5.21 | 0 | 0.0019 | 0.0013 | 0.23 | 0.039 |
| 2658632 | 0.66 | 0.0001 | 1.33 | 0.65 | 0 | 0.66 | 0.64 | 0.66 | 0.65 | 4.88 | 0 | 0 | 0.0001 | 0.02 | 0.002 |
| 2663553 | 0.61 | 0.0056 | 1.4 | 0.61 | 0.0018 | 0.5 | 0.73 | 0.66 | 0.59 | 4.84 | 0.0002 | 0.0024 | 0.0065 | 0.08 | 0.092 |
| 2666503 | 0.61 | 0.0086 | 1.34 | 0.63 | 0.0002 | 0.58 | 0.68 | 0.66 | 0.61 | 5.98 | 0.0002 | 0.0068 | 0.0069 | 0.45 | 0.125 |
| 2666519 | 0.62 | 0.0027 | 1.26 | 0.62 | 0.0007 | 0.52 | 0.72 | 0.66 | 0.6 | 6.48 | 0.0007 | 0.003 | 0.0025 | 0.28 | 0.053 |
| 2666524 | 0.62 | 0.003 | 1.42 | 0.6 | 0.0064 | 0.54 | 0.65 | 0.62 | 0.58 | 4.57 | 0.0053 | 0.0014 | 0.0021 | 0.21 | 0.036 |
| 2669316 | 0.6 | 0.0107 | 1.45 | 0.61 | 0.0028 | 0.55 | 0.66 | 0.63 | 0.59 | 4.88 | 0.0028 | 0.008 | 0.0072 | 0.34 | 0.119 |
| 2669571 | 0.64 | 0.001 | 1.33 | 0.63 | 0.0004 | 0.59 | 0.66 | 0.65 | 0.61 | 5.12 | 0.0011 | 0.0057 | 0.0023 | 0.07 | 0.012 |
| 2670438 | 0.62 | 0.0034 | 0.79 | 0.37 | 1 | 0.43 | 0.32 | 0.39 | 0.35 | 4.25 | 0.0015 | 0.0286 | 0.0075 | 0.28 | 0.004 |
| 2672775 | 0.61 | 0.0088 | 1.43 | 0.59 | 0.0095 | 0.63 | 0.55 | 0.59 | 0.59 | 4.95 | 0.0084 | 0.0064 | 0.0081 | 0.11 | 0.022 |
| 2674243 | 0.6 | 0.0141 | 1.33 | 0.62 | 0.0007 | 0.71 | 0.53 | 0.61 | 0.64 | 5.72 | 0.0008 | 0.0118 | 0.009 | 0.08 | 0.008 |
| 2675269 | 0.62 | 0.0041 | 1.39 | 0.58 | 0.0276 | 0.48 | 0.68 | 0.61 | 0.56 | 4.13 | 0.017 | 0.0135 | 0.0141 | 0.2 | 0.045 |
| 2675802 | 0.62 | 0.0039 | 1.34 | 0.61 | 0.0018 | 0.58 | 0.64 | 0.63 | 0.6 | 5.78 | 0.0016 | 0.0034 | 0.0028 | 0.13 | 0.032 |
| 2676931 | 0.6 | 0.0124 | 1.33 | 0.6 | 0.0042 | 0.59 | 0.61 | 0.61 | 0.59 | 6.33 | 0.0046 | 0.0174 | 0.0107 | 0.28 | 0.029 |
| 2677655 | 0.63 | 0.0015 | 1.31 | 0.65 | 0 | 0.66 | 0.64 | 0.66 | 0.65 | 5.9 | 0 | 0.0031 | 0.0013 | 0.14 | 0.006 |
| 2677695 | 0.68 | 0 | 1.37 | 0.61 | 0.0028 | 0.48 | 0.74 | 0.66 | 0.58 | 4.48 | 0.0007 | 0 | 0.0001 | 0 | 0 |
| 2680511 | 0.62 | 0.0024 | 1.26 | 0.63 | 0.0004 | 0.6 | 0.65 | 0.64 | 0.62 | 3.39 | 0.0001 | 0.0064 | 0.0067 | 0.03 | 0.018 |
| 2682663 | 0.63 | 0.0021 | 1.29 | 0.65 | 0 | 0.58 | 0.72 | 0.69 | 0.63 | 6.34 | 0 | 0.0023 | 0.0018 | 0.14 | 0.011 |
| 2685954 | 0.6 | 0.0132 | 1.25 | 0.57 | 0.0513 | 0.44 | 0.7 | 0.6 | 0.55 | 3.66 | 0.031 | 0.0321 | 0.0608 | 0.15 | 0.125 |
| 2691756 | 0.64 | 0.0007 | 1.27 | 0.62 | 0.0007 | 0.6 | 0.64 | 0.64 | 0.61 | 4.72 | 0.0003 | 0.0011 | 0.0011 | 0 | 0.001 |
| 2692111 | 0.64 | 0.0004 | 1.51 | 0.64 | 0.0001 | 0.59 | 0.68 | 0.66 | 0.62 | 5.09 | 0.0001 | 0.0009 | 0.0006 | 0.03 | 0.009 |
| 2694407 | 0.65 | 0.0004 | 1.28 | 0.59 | 0.0138 | 0.54 | 0.63 | 0.6 | 0.57 | 4 | 0.0096 | 0.0005 | 0.001 | 0.02 | 0.008 |
| 2694648 | 0.62 | 0.0049 | 1.33 | 0.6 | 0.0042 | 0.64 | 0.56 | 0.6 | 0.6 | 5.51 | 0.0049 | 0.0047 | 0.0042 | 0.05 | 0.011 |
| 2694649 | 0.64 | 0.0009 | 1.44 | 0.61 | 0.0018 | 0.61 | 0.61 | 0.62 | 0.61 | 5.62 | 0.0025 | 0.0013 | 0.0012 | 0.1 | 0.015 |
| 2695413 | 0.66 | 0.0001 | 1.25 | 0.62 | 0.0011 | 0.53 | 0.7 | 0.65 | 0.59 | 5.99 | 0.0007 | 0.0004 | 0.0002 | 0.07 | 0.005 |
| 2700507 | 0.62 | 0.0026 | 1.29 | 0.56 | 0.0681 | 0.46 | 0.67 | 0.59 | 0.55 | 5.39 | 0.0471 | 0.002 | 0.0035 | 0.13 | 0.146 |
| 2700829 | 0.64 | 0.0009 | 1.35 | 0.59 | 0.0095 | 0.53 | 0.65 | 0.61 | 0.58 | 5.18 | 0.0112 | 0.0027 | 0.0026 | 0.03 | 0.004 |
| 2701289 | 0.62 | 0.0026 | 1.26 | 0.63 | 0.0004 | 0.61 | 0.64 | 0.64 | 0.62 | 5.43 | 0.0004 | 0.0061 | 0.0033 | 0.2 | 0.026 |
| 2702329 | 0.61 | 0.0058 | 1.37 | 0.6 | 0.0042 | 0.57 | 0.63 | 0.62 | 0.59 | 4.76 | 0.0026 | 0.0048 | 0.0058 | 0.31 | 0.103 |
| 2703240 | 0.63 | 0.002 | 1.55 | 0.6 | 0.0064 | 0.47 | 0.73 | 0.64 | 0.57 | 4.57 | 0.0025 | 0.0021 | 0.0033 | 0.06 | 0.02 |
| 2704511 | 0.67 | 0 | 1.26 | 0.65 | 0 | 0.56 | 0.73 | 0.69 | 0.62 | 3.5 | 0 | 0 | 0 | 0.02 | 0.001 |
| 2704898 | 0.64 | 0.0006 | 1.36 | 0.59 | 0.0095 | 0.51 | 0.67 | 0.62 | 0.57 | 4.15 | 0.0071 | 0.0009 | 0.0009 | 0.12 | 0.01 |
| 2704935 | 0.63 | 0.002 | 1.25 | 0.63 | 0.0004 | 0.62 | 0.63 | 0.64 | 0.62 | 7.15 | 0.0003 | 0.0006 | 0.0011 | 0.03 | 0.015 |
| 2705001 | 0.66 | 0.0001 | 1.48 | 0.63 | 0.0002 | 0.58 | 0.68 | 0.66 | 0.61 | 6.26 | 0.0002 | 0 | 0.0001 | 0.01 | 0.001 |
| 2706322 | 0.61 | 0.0062 | 1.34 | 0.62 | 0.0011 | 0.59 | 0.64 | 0.63 | 0.61 | 5.01 | 0.0005 | 0.0044 | 0.0053 | 0.24 | 0.057 |
| 2706324 | 0.61 | 0.0103 | 1.29 | 0.61 | 0.0028 | 0.58 | 0.63 | 0.62 | 0.6 | 6.1 | 0.0029 | 0.0068 | 0.0076 | 0.09 | 0.014 |
| 2707780 | 0.62 | 0.0029 | 1.35 | 0.59 | 0.0095 | 0.55 | 0.63 | 0.61 | 0.58 | 5.34 | 0.0181 | 0.003 | 0.003 | 0.21 | 0.033 |
| 2708798 | 0.64 | 0.0007 | 1.33 | 0.62 | 0.0007 | 0.65 | 0.59 | 0.62 | 0.62 | 7.58 | 0.0012 | 0.0015 | 0.0017 | 0.07 | 0.007 |
| 2709489 | 0.64 | 0.0006 | 1.33 | 0.62 | 0.0007 | 0.54 | 0.7 | 0.65 | 0.6 | 5.65 | 0.0001 | 0.0012 | 0.0013 | 0.03 | 0.011 |
| 2710214 | 0.62 | 0.0049 | 1.26 | 0.59 | 0.0095 | 0.52 | 0.66 | 0.62 | 0.58 | 4.4 | 0.0132 | 0.0067 | 0.0045 | 0.59 | 0.087 |
| 2710217 | 0.67 | 0 | 1.37 | 0.64 | 0.0001 | 0.49 | 0.81 | 0.72 | 0.6 | 5.18 | 0 | 0 | 0 | 0.06 | 0.002 |
| 2711689 | 0.62 | 0.0029 | 1.26 | 0.6 | 0.0064 | 0.5 | 0.69 | 0.63 | 0.58 | 4.5 | 0.0032 | 0.0015 | 0.0042 | 0.06 | 0.035 |
| 2712424 | 0.62 | 0.0041 | 1.29 | 0.62 | 0.0011 | 0.64 | 0.59 | 0.62 | 0.62 | 6.2 | 0.0015 | 0.0033 | 0.003 | 0.11 | 0.012 |
| 2713075 | 0.61 | 0.0078 | 1.36 | 0.61 | 0.0028 | 0.64 | 0.57 | 0.61 | 0.61 | 6.22 | 0.0019 | 0.0061 | 0.0057 | 0.11 | 0.017 |
| 2713262 | 0.64 | 0.0007 | 1.31 | 0.64 | 0.0001 | 0.58 | 0.69 | 0.66 | 0.62 | 4.43 | 0.0001 | 0.0008 | 0.0014 | 0.05 | 0.011 |
| 2713269 | 0.66 | 0.0001 | 1.35 | 0.62 | 0.0007 | 0.54 | 0.7 | 0.65 | 0.6 | 5.51 | 0.0003 | 0.0002 | 0.0002 | 0.07 | 0.005 |
| 2714106 | 0.61 | 0.0082 | 1.32 | 0.59 | 0.0095 | 0.54 | 0.64 | 0.61 | 0.58 | 4.38 | 0.0105 | 0.0303 | 0.017 | 0.32 | 0.039 |
| 2715470 | 0.61 | 0.0068 | 1.41 | 0.6 | 0.0064 | 0.66 | 0.53 | 0.59 | 0.6 | 5.57 | 0.0084 | 0.006 | 0.005 | 0.07 | 0.009 |
| 2715717 | 0.62 | 0.0027 | 1.58 | 0.61 | 0.0028 | 0.67 | 0.54 | 0.6 | 0.62 | 5.55 | 0.003 | 0.0036 | 0.0026 | 0.06 | 0.007 |
| 2720693 | 0.63 | 0.0014 | 1.37 | 0.59 | 0.0138 | 0.43 | 0.76 | 0.64 | 0.56 | 4.6 | 0.0053 | 0.0039 | 0.0035 | 0.12 | 0.065 |
| 2724050 | 0.64 | 0.0008 | 1.28 | 0.61 | 0.0028 | 0.5 | 0.71 | 0.65 | 0.58 | 5.76 | 0.0009 | 0.001 | 0.0012 | 0.05 | 0.012 |
| 2724565 | 0.66 | 0.0001 | 1.29 | 0.62 | 0.0011 | 0.65 | 0.58 | 0.62 | 0.62 | 8.25 | 0.0011 | 0.0001 | 0.0001 | 0.02 | 0.003 |
| 2726514 | 0.64 | 0.0005 | 1.34 | 0.61 | 0.0028 | 0.55 | 0.66 | 0.63 | 0.59 | 4.4 | 0.0022 | 0.0026 | 0.0013 | 0.05 | 0.006 |
| 2727970 | 0.62 | 0.0039 | 1.49 | 0.63 | 0.0004 | 0.63 | 0.62 | 0.63 | 0.62 | 5.1 | 0.0004 | 0.0037 | 0.0023 | 0.11 | 0.02 |
| 2730701 | 0.63 | 0.0014 | 1.27 | 0.58 | 0.0276 | 0.5 | 0.66 | 0.6 | 0.56 | 4.34 | 0.0546 | 0.0052 | 0.0028 | 0.16 | 0.025 |
| 2731872 | 0.61 | 0.0067 | 1.31 | 0.58 | 0.0197 | 0.44 | 0.73 | 0.63 | 0.56 | 4.68 | 0.0137 | 0.0064 | 0.006 | 0.15 | 0.039 |
| 2732403 | 0.62 | 0.0029 | 1.34 | 0.6 | 0.0064 | 0.59 | 0.6 | 0.61 | 0.59 | 4.85 | 0.0092 | 0.0024 | 0.0043 | 0.07 | 0.037 |
| 2735017 | 0.64 | 0.0005 | 1.37 | 0.59 | 0.0095 | 0.47 | 0.72 | 0.64 | 0.57 | 5.03 | 0.0071 | 0.0023 | 0.0012 | 0.41 | 0.085 |
| 2736295 | 0.63 | 0.0015 | 1.26 | 0.57 | 0.038 | 0.47 | 0.68 | 0.6 | 0.55 | 4.72 | 0.0306 | 0.0028 | 0.0034 | 0.16 | 0.058 |
| 2738963 | 0.62 | 0.0029 | 1.27 | 0.6 | 0.0042 | 0.54 | 0.66 | 0.63 | 0.59 | 5.3 | 0.0058 | 0.0042 | 0.0039 | 0.05 | 0.01 |
| 2743194 | 0.67 | 0 | 1.29 | 0.65 | 0 | 0.66 | 0.63 | 0.65 | 0.65 | 6.83 | 0.0001 | 0 | 0.0001 | 0.01 | 0.001 |
| 2744632 | 0.6 | 0.0148 | 1.25 | 0.6 | 0.0042 | 0.49 | 0.72 | 0.64 | 0.58 | 4.28 | 0.0023 | 0.0143 | 0.0221 | 0.19 | 0.352 |
| 2745687 | 0.61 | 0.0063 | 1.28 | 0.6 | 0.0064 | 0.54 | 0.65 | 0.62 | 0.58 | 5.03 | 0.0098 | 0.0112 | 0.008 | 0.22 | 0.035 |
| 2754300 | 0.64 | 0.0009 | 1.3 | 0.61 | 0.0018 | 0.52 | 0.7 | 0.65 | 0.59 | 4.52 | 0.0033 | 0.0064 | 0.0023 | 0.45 | 0.075 |
| 2757037 | 0.62 | 0.0027 | 1.29 | 0.62 | 0.0011 | 0.7 | 0.53 | 0.61 | 0.63 | 7.15 | 0.0011 | 0.0031 | 0.0035 | 0.04 | 0.005 |
| 2763813 | 0.6 | 0.0108 | 1.35 | 0.58 | 0.0197 | 0.45 | 0.72 | 0.63 | 0.56 | 4.04 | 0.0062 | 0.0072 | 0.0128 | 0.18 | 0.15 |
| 2766634 | 0.64 | 0.0006 | 1.31 | 0.63 | 0.0002 | 0.65 | 0.61 | 0.63 | 0.63 | 4.74 | 0.0004 | 0.0013 | 0.0013 | 0.08 | 0.014 |
| 2770195 | 0.6 | 0.0144 | 1.25 | 0.54 | 0.2178 | 0.45 | 0.63 | 0.56 | 0.53 | 3.41 | 0.3483 | 0.0313 | 0.0237 | 0.19 | 0.027 |
| 2770473 | 0.63 | 0.0012 | 1.29 | 0.62 | 0.0007 | 0.57 | 0.67 | 0.64 | 0.61 | 6.83 | 0.0008 | 0.0014 | 0.0009 | 0.21 | 0.02 |
| 2771759 | 0.63 | 0.0022 | 1.27 | 0.6 | 0.0042 | 0.52 | 0.68 | 0.63 | 0.58 | 5.04 | 0.0084 | 0.0049 | 0.0025 | 0.04 | 0.005 |
| 2773129 | 0.63 | 0.0011 | 1.34 | 0.59 | 0.0138 | 0.62 | 0.55 | 0.59 | 0.59 | 4.18 | 0.0179 | 0.0023 | 0.0014 | 0.12 | 0.027 |
| 2774054 | 0.62 | 0.0048 | 1.33 | 0.6 | 0.0042 | 0.49 | 0.72 | 0.64 | 0.58 | 4.26 | 0.0032 | 0.0173 | 0.0105 | 0.17 | 0.023 |
| 2775592 | 0.62 | 0.0034 | 1.26 | 0.6 | 0.0042 | 0.59 | 0.61 | 0.61 | 0.59 | 5.07 | 0.0035 | 0.0017 | 0.0031 | 0.12 | 0.046 |
| 2775758 | 0.61 | 0.007 | 1.32 | 0.62 | 0.0011 | 0.7 | 0.53 | 0.61 | 0.63 | 6.83 | 0.0015 | 0.0111 | 0.0091 | 0.13 | 0.015 |
| 2776094 | 0.61 | 0.0083 | 1.27 | 0.6 | 0.0064 | 0.51 | 0.68 | 0.63 | 0.58 | 4.3 | 0.0137 | 0.0369 | 0.0113 | 0.2 | 0.01 |
| 2777492 | 0.6 | 0.0141 | 1.27 | 0.56 | 0.0889 | 0.39 | 0.73 | 0.6 | 0.54 | 4.01 | 0.0641 | 0.0153 | 0.0223 | 0.22 | 0.077 |
| 2779488 | 0.63 | 0.0012 | 1.25 | 0.63 | 0.0004 | 0.61 | 0.64 | 0.64 | 0.62 | 6.49 | 0.0003 | 0.0009 | 0.0011 | 0.04 | 0.006 |
| 2779639 | 0.63 | 0.0014 | 1.45 | 0.61 | 0.0018 | 0.63 | 0.59 | 0.62 | 0.61 | 5.62 | 0.0018 | 0.0019 | 0.0012 | 0.05 | 0.005 |
| 2779995 | 0.61 | 0.0056 | 1.26 | 0.61 | 0.0028 | 0.56 | 0.65 | 0.63 | 0.59 | 5.44 | 0.0037 | 0.005 | 0.0035 | 0.12 | 0.014 |
| 2780545 | 0.62 | 0.0033 | 1.4 | 0.61 | 0.0028 | 0.55 | 0.66 | 0.63 | 0.59 | 4.57 | 0.0035 | 0.0062 | 0.0052 | 0.17 | 0.037 |
| 2783475 | 0.64 | 0.0006 | 1.32 | 0.64 | 0.0001 | 0.73 | 0.54 | 0.62 | 0.66 | 6.56 | 0.0003 | 0.0012 | 0.0005 | 0.05 | 0.001 |
| 2789554 | 0.61 | 0.0079 | 1.27 | 0.59 | 0.0095 | 0.5 | 0.69 | 0.63 | 0.57 | 5.86 | 0.0085 | 0.0066 | 0.0081 | 0.1 | 0.072 |
| 2790573 | 0.61 | 0.0069 | 1.28 | 0.61 | 0.0018 | 0.65 | 0.57 | 0.61 | 0.62 | 6.28 | 0.0029 | 0.0076 | 0.0037 | 0.04 | 0.004 |
| 2797449 | 0.64 | 0.0007 | 1.28 | 0.62 | 0.0007 | 0.55 | 0.69 | 0.65 | 0.6 | 6.42 | 0.0004 | 0.0011 | 0.0009 | 0.07 | 0.013 |
| 2801338 | 0.62 | 0.0034 | 1.43 | 0.61 | 0.0028 | 0.52 | 0.69 | 0.64 | 0.59 | 4.71 | 0.0012 | 0.0021 | 0.0032 | 0.12 | 0.045 |
| 2801556 | 0.63 | 0.0022 | 1.3 | 0.61 | 0.0028 | 0.64 | 0.57 | 0.61 | 0.61 | 6.84 | 0.0027 | 0.0014 | 0.0016 | 0.01 | 0.004 |
| 2801679 | 0.63 | 0.0014 | 1.32 | 0.61 | 0.0028 | 0.61 | 0.6 | 0.61 | 0.6 | 6.25 | 0.0027 | 0.0007 | 0.0014 | 0.01 | 0.005 |
| 2802506 | 0.64 | 0.0004 | 1.3 | 0.63 | 0.0004 | 0.62 | 0.63 | 0.64 | 0.62 | 4.57 | 0.0001 | 0.0002 | 0.001 | 0.01 | 0.017 |
| 2805739 | 0.62 | 0.004 | 1.28 | 0.59 | 0.0095 | 0.5 | 0.68 | 0.62 | 0.57 | 4.4 | 0.0082 | 0.006 | 0.0061 | 0.09 | 0.024 |
| 2806859 | 0.66 | 0.0001 | 1.28 | 0.62 | 0.0007 | 0.54 | 0.7 | 0.65 | 0.6 | 6.23 | 0.0004 | 0.0001 | 0.0001 | 0.01 | 0.002 |
| 2806912 | 0.62 | 0.0032 | 1.3 | 0.6 | 0.0042 | 0.53 | 0.67 | 0.63 | 0.58 | 6.08 | 0.0043 | 0.0037 | 0.003 | 0.14 | 0.019 |
| 2807658 | 0.64 | 0.0005 | 1.27 | 0.6 | 0.0064 | 0.5 | 0.69 | 0.63 | 0.58 | 5.22 | 0.0033 | 0.0007 | 0.0008 | 0.07 | 0.008 |
| 2809146 | 0.61 | 0.0071 | 1.43 | 0.62 | 0.0011 | 0.61 | 0.62 | 0.63 | 0.61 | 5.52 | 0.0018 | 0.0083 | 0.0055 | 0.14 | 0.037 |
| 2810487 | 0.6 | 0.0114 | 1.26 | 0.61 | 0.0028 | 0.55 | 0.66 | 0.63 | 0.59 | 4.07 | 0.0007 | 0.0066 | 0.0152 | 0.3 | 0.315 |
| 2812307 | 0.61 | 0.0075 | 1.37 | 0.59 | 0.0138 | 0.5 | 0.67 | 0.61 | 0.57 | 4.53 | 0.0223 | 0.0159 | 0.0104 | 0.13 | 0.02 |
| 2812500 | 0.63 | 0.0017 | 1.33 | 0.57 | 0.038 | 0.46 | 0.69 | 0.61 | 0.55 | 3.64 | 0.0507 | 0.0039 | 0.0034 | 0.17 | 0.023 |
| 2812573 | 0.63 | 0.0013 | 1.25 | 0.6 | 0.0064 | 0.6 | 0.59 | 0.6 | 0.59 | 7.18 | 0.0067 | 0.0014 | 0.0011 | 0.07 | 0.006 |
| 2817404 | 0.61 | 0.0067 | 1.49 | 0.61 | 0.0028 | 0.53 | 0.68 | 0.64 | 0.59 | 4.52 | 0.0034 | 0.009 | 0.007 | 0.29 | 0.036 |
| 2817457 | 0.64 | 0.0007 | 1.5 | 0.6 | 0.0064 | 0.51 | 0.68 | 0.63 | 0.58 | 5.07 | 0.0037 | 0.0015 | 0.0011 | 0.03 | 0.007 |
| 2818271 | 0.6 | 0.0132 | 1.25 | 0.63 | 0.0002 | 0.57 | 0.69 | 0.66 | 0.61 | 6.77 | 0.0002 | 0.022 | 0.0269 | 0.47 | 0.07 |
| 2818964 | 0.63 | 0.0019 | 1.37 | 0.63 | 0.0002 | 0.71 | 0.55 | 0.62 | 0.65 | 6.65 | 0.0002 | 0.0025 | 0.002 | 0.06 | 0.006 |
| 2822274 | 0.6 | 0.0113 | 1.43 | 0.58 | 0.0276 | 0.5 | 0.66 | 0.6 | 0.56 | 5.18 | 0.0299 | 0.0195 | 0.0143 | 0.15 | 0.035 |
| 2823066 | 0.64 | 0.0007 | 1.33 | 0.61 | 0.0028 | 0.44 | 0.79 | 0.68 | 0.57 | 3.93 | 0.0011 | 0.0034 | 0.0021 | 0.24 | 0.062 |
| 2823854 | 0.61 | 0.0066 | 1.43 | 0.6 | 0.0042 | 0.62 | 0.58 | 0.61 | 0.6 | 6.28 | 0.003 | 0.0028 | 0.0036 | 0.09 | 0.026 |
| 2824899 | 0.63 | 0.0021 | 1.31 | 0.61 | 0.0028 | 0.52 | 0.69 | 0.64 | 0.59 | 4.62 | 0.0015 | 0.0025 | 0.0045 | 0.1 | 0.034 |
| 2828112 | 0.63 | 0.002 | 1.54 | 0.62 | 0.0007 | 0.66 | 0.58 | 0.62 | 0.63 | 5.66 | 0.0006 | 0.0016 | 0.0019 | 0.04 | 0.007 |
| 2828637 | 0.6 | 0.0123 | 1.3 | 0.61 | 0.0018 | 0.55 | 0.67 | 0.64 | 0.59 | 5.38 | 0.0004 | 0.0021 | 0.007 | 0.13 | 0.068 |
| 2828642 | 0.63 | 0.0019 | 1.42 | 0.59 | 0.0095 | 0.53 | 0.65 | 0.61 | 0.58 | 4.58 | 0.0105 | 0.002 | 0.0024 | 0.13 | 0.023 |
| 2829255 | 0.64 | 0.0008 | 1.28 | 0.62 | 0.0011 | 0.47 | 0.78 | 0.68 | 0.58 | 5.09 | 0.0007 | 0.0012 | 0.0011 | 0.09 | 0.024 |
| 2829555 | 0.61 | 0.0076 | 1.26 | 0.59 | 0.0095 | 0.48 | 0.71 | 0.63 | 0.57 | 5.17 | 0.0037 | 0.0043 | 0.0066 | 0.12 | 0.058 |
| 2829679 | 0.62 | 0.0037 | 1.26 | 0.61 | 0.0028 | 0.58 | 0.63 | 0.62 | 0.6 | 5.77 | 0.0035 | 0.0021 | 0.0024 | 0.05 | 0.007 |
| 2831231 | 0.62 | 0.0034 | 1.25 | 0.58 | 0.0276 | 0.52 | 0.63 | 0.6 | 0.56 | 6.06 | 0.0213 | 0.004 | 0.0045 | 0.1 | 0.034 |
| 2831557 | 0.63 | 0.0011 | 1.38 | 0.61 | 0.0028 | 0.61 | 0.6 | 0.61 | 0.6 | 6.54 | 0.0037 | 0.0009 | 0.0006 | 0.02 | 0.001 |
| 2836687 | 0.61 | 0.0085 | 1.29 | 0.6 | 0.0064 | 0.63 | 0.56 | 0.6 | 0.6 | 4.96 | 0.006 | 0.0061 | 0.0074 | 0.03 | 0.015 |
| 2840638 | 0.61 | 0.0057 | 1.33 | 0.61 | 0.0028 | 0.6 | 0.61 | 0.62 | 0.6 | 4.59 | 0.0023 | 0.0053 | 0.0072 | 0.2 | 0.032 |
| 2843280 | 0.61 | 0.0099 | 1.37 | 0.61 | 0.0028 | 0.64 | 0.57 | 0.61 | 0.61 | 5.82 | 0.0017 | 0.0084 | 0.0073 | 0.03 | 0.009 |
| 2844248 | 0.61 | 0.0103 | 1.38 | 0.62 | 0.0011 | 0.64 | 0.59 | 0.62 | 0.62 | 5.94 | 0.0022 | 0.008 | 0.0046 | 0.09 | 0.01 |
| 2844250 | 0.61 | 0.0063 | 1.36 | 0.59 | 0.0095 | 0.65 | 0.53 | 0.59 | 0.6 | 5.71 | 0.0115 | 0.0052 | 0.0043 | 0.1 | 0.014 |
| 2848258 | 0.62 | 0.0027 | 1.32 | 0.56 | 0.0889 | 0.44 | 0.68 | 0.59 | 0.54 | 5.15 | 0.0478 | 0.0024 | 0.0038 | 0.07 | 0.039 |
| 2848472 | 0.62 | 0.0042 | 1.6 | 0.65 | 0 | 0.63 | 0.66 | 0.66 | 0.64 | 5.69 | 0 | 0.0017 | 0.0018 | 0.03 | 0.009 |
| 2851973 | 0.64 | 0.0006 | 1.29 | 0.61 | 0.0028 | 0.56 | 0.65 | 0.63 | 0.59 | 4.78 | 0.002 | 0.0007 | 0.001 | 0.01 | 0.004 |
| 2854808 | 0.64 | 0.0009 | 1.58 | 0.63 | 0.0002 | 0.66 | 0.6 | 0.63 | 0.63 | 6.05 | 0.0003 | 0.0006 | 0.0008 | 0.05 | 0.007 |
| 2854809 | 0.63 | 0.0011 | 1.27 | 0.63 | 0.0004 | 0.61 | 0.64 | 0.64 | 0.62 | 8.1 | 0.0003 | 0.0006 | 0.0009 | 0.06 | 0.008 |
| 2855289 | 0.62 | 0.0024 | 1.29 | 0.58 | 0.0276 | 0.48 | 0.68 | 0.61 | 0.56 | 5.6 | 0.0568 | 0.0088 | 0.0057 | 0.05 | 0.006 |
| 2855291 | 0.63 | 0.0011 | 1.41 | 0.62 | 0.0011 | 0.53 | 0.7 | 0.65 | 0.59 | 4.74 | 0.0015 | 0.0034 | 0.0021 | 0.06 | 0.009 |
| 2855292 | 0.62 | 0.0044 | 1.27 | 0.62 | 0.0007 | 0.57 | 0.67 | 0.64 | 0.61 | 5.57 | 0.0013 | 0.009 | 0.006 | 0.08 | 0.011 |
| 2857172 | 0.62 | 0.0041 | 1.4 | 0.6 | 0.0064 | 0.58 | 0.61 | 0.61 | 0.59 | 5.05 | 0.0067 | 0.0035 | 0.0032 | 0.2 | 0.037 |
| 2862162 | 0.61 | 0.0079 | 1.28 | 0.62 | 0.0007 | 0.62 | 0.62 | 0.63 | 0.62 | 4.45 | 0.0004 | 0.0172 | 0.0192 | 0.16 | 0.049 |
| 2862377 | 0.61 | 0.0068 | 1.3 | 0.64 | 0.0001 | 0.66 | 0.61 | 0.64 | 0.64 | 7.95 | 0.0003 | 0.0059 | 0.0047 | 0.1 | 0.009 |
| 2865063 | 0.62 | 0.003 | 1.34 | 0.62 | 0.0011 | 0.65 | 0.58 | 0.62 | 0.62 | 6.34 | 0.0018 | 0.0028 | 0.0027 | 0.09 | 0.008 |
| 2868141 | 0.62 | 0.0029 | 1.28 | 0.63 | 0.0004 | 0.59 | 0.66 | 0.65 | 0.61 | 5.75 | 0.0002 | 0.0021 | 0.0033 | 0.04 | 0.006 |
| 2877518 | 0.61 | 0.0092 | 1.28 | 0.6 | 0.0042 | 0.62 | 0.58 | 0.61 | 0.6 | 5.41 | 0.0057 | 0.0083 | 0.0089 | 0.16 | 0.042 |
| 2878261 | 0.62 | 0.0029 | 1.29 | 0.63 | 0.0002 | 0.63 | 0.63 | 0.64 | 0.63 | 5.98 | 0.0001 | 0.0011 | 0.0025 | 0.05 | 0.015 |
| 2882121 | 0.63 | 0.001 | 1.26 | 0.6 | 0.0042 | 0.57 | 0.63 | 0.62 | 0.59 | 7.76 | 0.0045 | 0.0009 | 0.0015 | 0.04 | 0.004 |
| 2888344 | 0.6 | 0.0148 | 1.3 | 0.57 | 0.038 | 0.51 | 0.63 | 0.59 | 0.56 | 4.55 | 0.05 | 0.0191 | 0.0135 | 0.2 | 0.072 |
| 2893822 | 0.61 | 0.0089 | 1.26 | 0.59 | 0.0095 | 0.45 | 0.74 | 0.64 | 0.57 | 4.91 | 0.0028 | 0.01 | 0.0211 | 0.08 | 0.045 |
| 2893942 | 0.61 | 0.0078 | 1.31 | 0.58 | 0.0276 | 0.53 | 0.62 | 0.59 | 0.56 | 6.54 | 0.0371 | 0.0132 | 0.0084 | 0.14 | 0.019 |
| 2896978 | 0.62 | 0.0027 | 1.36 | 0.59 | 0.0138 | 0.48 | 0.7 | 0.62 | 0.57 | 4.8 | 0.0165 | 0.0112 | 0.0042 | 0.2 | 0.011 |
| 2898531 | 0.63 | 0.001 | 1.28 | 0.58 | 0.0276 | 0.67 | 0.48 | 0.57 | 0.59 | 5.33 | 0.0173 | 0.0012 | 0.0016 | 0.05 | 0.009 |
| 2902010 | 0.65 | 0.0003 | 1.26 | 0.63 | 0.0002 | 0.58 | 0.68 | 0.66 | 0.61 | 6.44 | 0.0002 | 0.0002 | 0.0003 | 0.05 | 0.005 |
| 2902011 | 0.62 | 0.0024 | 1.45 | 0.63 | 0.0004 | 0.68 | 0.57 | 0.62 | 0.64 | 5.66 | 0.0007 | 0.0014 | 0.0013 | 0.07 | 0.01 |
| 2905137 | 0.63 | 0.0022 | 1.31 | 0.6 | 0.0042 | 0.47 | 0.74 | 0.65 | 0.57 | 4.98 | 0.002 | 0.0011 | 0.0016 | 0.01 | 0.002 |
| 2906968 | 0.61 | 0.0101 | 1.28 | 0.6 | 0.0064 | 0.57 | 0.62 | 0.61 | 0.59 | 5.8 | 0.0118 | 0.0121 | 0.0081 | 0.15 | 0.011 |
| 2908048 | 0.62 | 0.0041 | 1.27 | 0.62 | 0.0011 | 0.7 | 0.53 | 0.61 | 0.63 | 4.51 | 0.0008 | 0.005 | 0.0058 | 0.07 | 0.031 |
| 2909471 | 0.6 | 0.014 | 1.26 | 0.58 | 0.0276 | 0.55 | 0.6 | 0.59 | 0.57 | 5 | 0.0413 | 0.0243 | 0.019 | 0.28 | 0.07 |
| 2910489 | 0.63 | 0.0015 | 1.25 | 0.61 | 0.0028 | 0.6 | 0.61 | 0.62 | 0.6 | 5.4 | 0.0045 | 0.0036 | 0.0026 | 0.05 | 0.008 |
| 2914002 | 0.64 | 0.0008 | 1.31 | 0.59 | 0.0095 | 0.47 | 0.72 | 0.64 | 0.57 | 4.56 | 0.0045 | 0.0015 | 0.0016 | 0.02 | 0.003 |
| 2914107 | 0.67 | 0 | 1.26 | 0.65 | 0 | 0.54 | 0.76 | 0.7 | 0.62 | 4.52 | 0.0001 | 0.0006 | 0.0006 | 0.06 | 0.009 |
| 2916383 | 0.61 | 0.0088 | 1.26 | 0.58 | 0.0197 | 0.62 | 0.54 | 0.58 | 0.58 | 5.79 | 0.0218 | 0.0129 | 0.0103 | 0.37 | 0.063 |
| 2916976 | 0.61 | 0.0055 | 1.26 | 0.59 | 0.0095 | 0.48 | 0.71 | 0.63 | 0.57 | 4.23 | 0.003 | 0.007 | 0.0089 | 0.16 | 0.036 |
| 2918543 | 0.63 | 0.0012 | 1.3 | 0.62 | 0.0007 | 0.6 | 0.64 | 0.64 | 0.61 | 4.74 | 0.0004 | 0.0036 | 0.0022 | 0.09 | 0.012 |
| 2918547 | 0.64 | 0.0006 | 1.38 | 0.61 | 0.0018 | 0.58 | 0.64 | 0.63 | 0.6 | 6.5 | 0.0013 | 0.0005 | 0.0005 | 0.04 | 0.003 |
| 2922232 | 0.63 | 0.0014 | 1.27 | 0.6 | 0.0042 | 0.5 | 0.71 | 0.64 | 0.58 | 5.15 | 0.003 | 0.0058 | 0.003 | 0.42 | 0.047 |
| 2922235 | 0.61 | 0.0052 | 1.27 | 0.59 | 0.0095 | 0.49 | 0.7 | 0.63 | 0.57 | 4.54 | 0.0118 | 0.0449 | 0.0191 | 0.14 | 0.024 |
| 2926222 | 0.64 | 0.0005 | 1.42 | 0.62 | 0.0007 | 0.65 | 0.59 | 0.62 | 0.62 | 7.41 | 0.0007 | 0.0005 | 0.0004 | 0.03 | 0.002 |
| 2929946 | 0.63 | 0.0021 | 1.28 | 0.61 | 0.0028 | 0.5 | 0.71 | 0.65 | 0.58 | 5.67 | 0.0008 | 0.0018 | 0.0024 | 0.11 | 0.034 |
| 2930652 | 0.64 | 0.0009 | 1.3 | 0.61 | 0.0018 | 0.6 | 0.62 | 0.62 | 0.6 | 6.46 | 0.0023 | 0.0023 | 0.001 | 0.17 | 0.016 |
| 2932399 | 0.63 | 0.0022 | 1.25 | 0.6 | 0.0064 | 0.51 | 0.68 | 0.63 | 0.58 | 4.77 | 0.0094 | 0.0045 | 0.0023 | 0.22 | 0.047 |
| 2932428 | 0.61 | 0.0057 | 1.37 | 0.58 | 0.0276 | 0.48 | 0.68 | 0.61 | 0.56 | 4.49 | 0.0327 | 0.0118 | 0.0092 | 0.14 | 0.043 |
| 2933660 | 0.64 | 0.0005 | 1.26 | 0.66 | 0 | 0.65 | 0.66 | 0.67 | 0.65 | 5.65 | 0 | 0.0009 | 0.0009 | 0.17 | 0.023 |
| 2936963 | 0.6 | 0.0118 | 1.31 | 0.57 | 0.0513 | 0.44 | 0.7 | 0.6 | 0.55 | 3.91 | 0.0437 | 0.0364 | 0.0354 | 0.56 | 0.204 |
| 2940827 | 0.62 | 0.0039 | 1.3 | 0.6 | 0.0064 | 0.57 | 0.62 | 0.61 | 0.59 | 5.47 | 0.0054 | 0.0081 | 0.0057 | 0.1 | 0.013 |
| 2942512 | 0.63 | 0.0014 | 1.26 | 0.58 | 0.0197 | 0.5 | 0.67 | 0.61 | 0.56 | 6.1 | 0.0094 | 0.0033 | 0.0028 | 0.09 | 0.032 |
| 2944070 | 0.63 | 0.0014 | 1.54 | 0.61 | 0.0018 | 0.6 | 0.62 | 0.62 | 0.6 | 5.64 | 0.0011 | 0.0019 | 0.0016 | 0.16 | 0.033 |
| 2944963 | 0.66 | 0.0001 | 1.27 | 0.62 | 0.0007 | 0.57 | 0.67 | 0.64 | 0.61 | 7.12 | 0.0008 | 0.0003 | 0.0003 | 0.02 | 0.002 |
| 2946479 | 0.63 | 0.0012 | 1.28 | 0.6 | 0.0042 | 0.5 | 0.7 | 0.64 | 0.58 | 4.37 | 0.0049 | 0.0062 | 0.0046 | 0.2 | 0.041 |
| 2947592 | 0.64 | 0.0005 | 1.33 | 0.63 | 0.0004 | 0.59 | 0.66 | 0.65 | 0.61 | 4.54 | 0.0003 | 0.0003 | 0.0005 | 0 | 0.001 |
| 2948592 | 0.63 | 0.0013 | 1.3 | 0.61 | 0.0028 | 0.63 | 0.58 | 0.61 | 0.61 | 5.64 | 0.0019 | 0.0013 | 0.0012 | 0.06 | 0.008 |
| 2948596 | 0.6 | 0.0126 | 1.25 | 0.59 | 0.0138 | 0.61 | 0.56 | 0.59 | 0.59 | 4.34 | 0.0226 | 0.0211 | 0.0164 | 0.07 | 0.018 |
| 2949053 | 0.63 | 0.0023 | 1.4 | 0.63 | 0.0004 | 0.62 | 0.63 | 0.64 | 0.62 | 4.81 | 0.0002 | 0.0014 | 0.0023 | 0.05 | 0.01 |
| 2949061 | 0.61 | 0.0103 | 1.36 | 0.61 | 0.0028 | 0.56 | 0.65 | 0.63 | 0.59 | 4.82 | 0.0016 | 0.0056 | 0.0094 | 0.22 | 0.129 |
| 2951060 | 0.61 | 0.0083 | 1.26 | 0.6 | 0.0042 | 0.65 | 0.55 | 0.6 | 0.61 | 6.61 | 0.0083 | 0.008 | 0.0046 | 0.09 | 0.006 |
| 2956077 | 0.63 | 0.0022 | 1.28 | 0.6 | 0.0042 | 0.5 | 0.7 | 0.64 | 0.58 | 5.37 | 0.0038 | 0.001 | 0.0026 | 0.05 | 0.039 |
| 2958402 | 0.65 | 0.0002 | 1.32 | 0.61 | 0.0018 | 0.52 | 0.7 | 0.65 | 0.59 | 5.05 | 0.0005 | 0.0009 | 0.0016 | 0.04 | 0.026 |
| 2961302 | 0.61 | 0.0086 | 1.32 | 0.63 | 0.0004 | 0.7 | 0.55 | 0.62 | 0.64 | 5.56 | 0.0007 | 0.0083 | 0.0078 | 0.09 | 0.025 |
| 2966293 | 0.63 | 0.002 | 1.33 | 0.62 | 0.0011 | 0.55 | 0.68 | 0.64 | 0.6 | 4.26 | 0.0006 | 0.0023 | 0.0019 | 0.17 | 0.013 |
| 2966378 | 0.62 | 0.0045 | 1.28 | 0.6 | 0.0042 | 0.62 | 0.58 | 0.61 | 0.6 | 5.81 | 0.0052 | 0.008 | 0.0048 | 0.2 | 0.039 |
| 2967164 | 0.62 | 0.0029 | 1.27 | 0.62 | 0.0007 | 0.53 | 0.71 | 0.66 | 0.6 | 4.09 | 0.0005 | 0.0074 | 0.0048 | 0.6 | 0.095 |
| 2967323 | 0.61 | 0.0085 | 1.33 | 0.57 | 0.038 | 0.49 | 0.66 | 0.6 | 0.56 | 4.67 | 0.0674 | 0.0113 | 0.0083 | 0.05 | 0.008 |
| 2968257 | 0.63 | 0.0011 | 1.28 | 0.62 | 0.0007 | 0.62 | 0.62 | 0.63 | 0.62 | 5.01 | 0.0003 | 0.0018 | 0.0016 | 0.03 | 0.014 |
| 2968653 | 0.61 | 0.0055 | 1.33 | 0.6 | 0.0042 | 0.56 | 0.64 | 0.62 | 0.59 | 5.14 | 0.0024 | 0.006 | 0.0053 | 0.21 | 0.025 |
| 2974195 | 0.61 | 0.0079 | 1.26 | 0.62 | 0.0011 | 0.7 | 0.53 | 0.61 | 0.63 | 6.73 | 0.0012 | 0.0135 | 0.0134 | 0.1 | 0.016 |
| 2974332 | 0.61 | 0.0066 | 1.53 | 0.6 | 0.0042 | 0.59 | 0.61 | 0.61 | 0.59 | 4.95 | 0.0103 | 0.0105 | 0.0062 | 0.07 | 0.011 |
| 2975686 | 0.63 | 0.0017 | 1.28 | 0.63 | 0.0004 | 0.63 | 0.62 | 0.63 | 0.62 | 5.93 | 0.0008 | 0.0034 | 0.0016 | 0.12 | 0.004 |
| 2975709 | 0.61 | 0.0069 | 1.28 | 0.57 | 0.0513 | 0.5 | 0.63 | 0.59 | 0.55 | 4.81 | 0.0755 | 0.014 | 0.0119 | 0.16 | 0.023 |
| 2976368 | 0.61 | 0.0066 | 1.38 | 0.62 | 0.0007 | 0.55 | 0.69 | 0.65 | 0.6 | 5.16 | 0.0003 | 0.011 | 0.0111 | 0.26 | 0.088 |
| 2977521 | 0.61 | 0.0096 | 1.28 | 0.61 | 0.0018 | 0.7 | 0.52 | 0.6 | 0.63 | 6.52 | 0.0025 | 0.0156 | 0.0105 | 0.11 | 0.01 |
| 2981918 | 0.61 | 0.0091 | 1.4 | 0.59 | 0.0095 | 0.5 | 0.68 | 0.62 | 0.57 | 5.26 | 0.0096 | 0.0156 | 0.0111 | 0.05 | 0.018 |
| 2982323 | 0.61 | 0.0076 | 1.33 | 0.6 | 0.0042 | 0.61 | 0.59 | 0.61 | 0.6 | 6.64 | 0.0043 | 0.0078 | 0.0077 | 0.18 | 0.028 |
| 2982385 | 0.63 | 0.0011 | 1.45 | 0.61 | 0.0028 | 0.59 | 0.62 | 0.62 | 0.6 | 4.39 | 0.0093 | 0.0032 | 0.0011 | 0.19 | 0.005 |
| 2984576 | 0.61 | 0.0075 | 1.32 | 0.58 | 0.0197 | 0.51 | 0.65 | 0.6 | 0.57 | 4.1 | 0.0341 | 0.0075 | 0.0049 | 0.32 | 0.069 |
| 2985813 | 0.62 | 0.0044 | 1.43 | 0.6 | 0.0042 | 0.47 | 0.74 | 0.65 | 0.57 | 4.6 | 0.0017 | 0.0139 | 0.0132 | 0.45 | 0.127 |
| 2985965 | 0.65 | 0.0002 | 1.27 | 0.63 | 0.0004 | 0.55 | 0.7 | 0.66 | 0.61 | 6.33 | 0.0003 | 0.0004 | 0.0003 | 0.03 | 0.001 |
| 2988895 | 0.65 | 0.0003 | 1.28 | 0.6 | 0.0064 | 0.51 | 0.68 | 0.63 | 0.58 | 4.59 | 0.0042 | 0.0012 | 0.0014 | 0.03 | 0.01 |
| 2988896 | 0.62 | 0.0035 | 1.26 | 0.59 | 0.0138 | 0.73 | 0.44 | 0.57 | 0.61 | 7.15 | 0.0195 | 0.0055 | 0.0033 | 0.11 | 0.008 |
| 2989720 | 0.65 | 0.0004 | 1.33 | 0.61 | 0.0028 | 0.59 | 0.62 | 0.62 | 0.6 | 4.81 | 0.0026 | 0.001 | 0.0005 | 0.17 | 0.018 |
| 2991248 | 0.6 | 0.0125 | 1.26 | 0.59 | 0.0095 | 0.55 | 0.63 | 0.61 | 0.58 | 7.21 | 0.0102 | 0.0105 | 0.0087 | 0.61 | 0.119 |
| 2991263 | 0.66 | 0.0001 | 1.3 | 0.64 | 0.0001 | 0.59 | 0.69 | 0.67 | 0.62 | 7.03 | 0.0001 | 0.0004 | 0.0003 | 0.21 | 0.012 |
| 2991264 | 0.64 | 0.0009 | 1.44 | 0.61 | 0.0018 | 0.53 | 0.69 | 0.64 | 0.59 | 5.75 | 0.0016 | 0.0023 | 0.0012 | 0.4 | 0.039 |
| 2993622 | 0.63 | 0.0019 | 1.27 | 0.61 | 0.0028 | 0.64 | 0.57 | 0.61 | 0.61 | 5.13 | 0.0047 | 0.0024 | 0.0023 | 0.06 | 0.01 |
| 2993644 | 0.62 | 0.0038 | 1.34 | 0.62 | 0.0007 | 0.69 | 0.55 | 0.61 | 0.64 | 4.61 | 0.0006 | 0.0025 | 0.0037 | 0.03 | 0.013 |
| 2993659 | 0.67 | 0 | 1.42 | 0.64 | 0.0001 | 0.61 | 0.67 | 0.66 | 0.63 | 5.71 | 0.0001 | 0.0001 | 0.0001 | 0.01 | 0.001 |
| 2994371 | 0.6 | 0.013 | 1.3 | 0.59 | 0.0138 | 0.51 | 0.66 | 0.61 | 0.57 | 3.82 | 0.0373 | 0.0501 | 0.0192 | 0.87 | 0.102 |
| 2995598 | 0.6 | 0.0117 | 1.27 | 0.59 | 0.0138 | 0.53 | 0.64 | 0.61 | 0.57 | 5.35 | 0.0202 | 0.0287 | 0.011 | 0.15 | 0.03 |
| 2997143 | 0.64 | 0.0008 | 1.27 | 0.59 | 0.0138 | 0.49 | 0.69 | 0.62 | 0.57 | 4.85 | 0.0092 | 0.0019 | 0.0028 | 0.14 | 0.035 |
| 3002667 | 0.61 | 0.0053 | 1.27 | 0.61 | 0.0028 | 0.56 | 0.65 | 0.63 | 0.59 | 6.14 | 0.0026 | 0.0052 | 0.008 | 0.14 | 0.067 |
| 3003180 | 0.61 | 0.0068 | 1.29 | 0.57 | 0.038 | 0.5 | 0.65 | 0.6 | 0.56 | 4.11 | 0.0332 | 0.0094 | 0.0068 | 0.42 | 0.07 |
| 3004652 | 0.65 | 0.0002 | 1.41 | 0.6 | 0.0064 | 0.5 | 0.69 | 0.63 | 0.58 | 4.86 | 0.0052 | 0.0014 | 0.0007 | 0.08 | 0.006 |
| 3011652 | 0.61 | 0.0057 | 1.25 | 0.6 | 0.0064 | 0.54 | 0.65 | 0.62 | 0.58 | 6.99 | 0.005 | 0.0031 | 0.0033 | 0.07 | 0.01 |
| 3011846 | 0.6 | 0.0118 | 1.26 | 0.59 | 0.0138 | 0.48 | 0.7 | 0.62 | 0.57 | 4.59 | 0.01 | 0.0056 | 0.0081 | 0.18 | 0.093 |
| 3012405 | 0.66 | 0.0001 | 1.28 | 0.65 | 0 | 0.61 | 0.68 | 0.67 | 0.63 | 6.96 | 0.0001 | 0.0001 | 0.0001 | 0.04 | 0.003 |
| 3012428 | 0.67 | 0 | 1.29 | 0.64 | 0.0001 | 0.59 | 0.68 | 0.66 | 0.62 | 5.6 | 0.0001 | 0 | 0.0001 | 0.02 | 0.003 |
| 3012438 | 0.62 | 0.0046 | 1.3 | 0.57 | 0.038 | 0.46 | 0.69 | 0.61 | 0.55 | 4.08 | 0.0341 | 0.0025 | 0.0029 | 0.11 | 0.047 |
| 3012452 | 0.63 | 0.0014 | 1.4 | 0.59 | 0.0095 | 0.5 | 0.68 | 0.62 | 0.57 | 4.98 | 0.0071 | 0.0009 | 0.0012 | 0.09 | 0.015 |
| 3012453 | 0.64 | 0.001 | 1.31 | 0.64 | 0.0001 | 0.59 | 0.69 | 0.67 | 0.62 | 6.58 | 0 | 0.0006 | 0.001 | 0.07 | 0.007 |
| 3013190 | 0.62 | 0.0039 | 1.28 | 0.61 | 0.0018 | 0.5 | 0.73 | 0.66 | 0.59 | 4.85 | 0.0014 | 0.0075 | 0.0059 | 0.16 | 0.045 |
| 3013468 | 0.66 | 0.0001 | 1.4 | 0.63 | 0.0004 | 0.59 | 0.66 | 0.65 | 0.61 | 5.57 | 0.0003 | 0.0002 | 0.0001 | 0.05 | 0.006 |
| 3013685 | 0.62 | 0.0033 | 1.26 | 0.61 | 0.0028 | 0.53 | 0.68 | 0.64 | 0.59 | 5.07 | 0.0021 | 0.0046 | 0.0027 | 0.04 | 0.008 |
| 3014422 | 0.62 | 0.0036 | 1.37 | 0.63 | 0.0004 | 0.53 | 0.72 | 0.67 | 0.6 | 3.79 | 0.0002 | 0.0033 | 0.0096 | 0.12 | 0.104 |
| 3015985 | 0.61 | 0.0063 | 1.25 | 0.61 | 0.0028 | 0.64 | 0.57 | 0.61 | 0.61 | 6.89 | 0.0034 | 0.0081 | 0.0084 | 0.09 | 0.015 |
| 3017629 | 0.6 | 0.0114 | 1.28 | 0.55 | 0.1438 | 0.37 | 0.73 | 0.59 | 0.53 | 3.64 | 0.1043 | 0.0347 | 0.0366 | 0.22 | 0.076 |
| 3017826 | 0.6 | 0.0122 | 1.36 | 0.6 | 0.0064 | 0.58 | 0.61 | 0.61 | 0.59 | 5.59 | 0.007 | 0.0129 | 0.0081 | 0.05 | 0.006 |
| 3021760 | 0.66 | 0.0001 | 1.33 | 0.64 | 0.0001 | 0.53 | 0.76 | 0.69 | 0.61 | 4.96 | 0 | 0.0001 | 0.0002 | 0 | 0.002 |
| 3022682 | 0.62 | 0.0024 | 1.3 | 0.62 | 0.0011 | 0.59 | 0.64 | 0.63 | 0.61 | 6.47 | 0.0007 | 0.0035 | 0.0022 | 0.02 | 0.002 |
| 3027555 | 0.63 | 0.0017 | 1.55 | 0.63 | 0.0004 | 0.7 | 0.55 | 0.62 | 0.64 | 6.41 | 0.0004 | 0.001 | 0.0009 | 0.02 | 0.002 |
| 3027926 | 0.63 | 0.0018 | 1.31 | 0.62 | 0.0007 | 0.56 | 0.68 | 0.65 | 0.6 | 5.93 | 0.0005 | 0.0013 | 0.0017 | 0.06 | 0.016 |
| 3027936 | 0.62 | 0.0049 | 1.34 | 0.61 | 0.0018 | 0.72 | 0.5 | 0.6 | 0.64 | 6.19 | 0.0027 | 0.0043 | 0.0024 | 0.11 | 0.013 |
| 3037391 | 0.63 | 0.0019 | 1.36 | 0.59 | 0.0095 | 0.47 | 0.72 | 0.64 | 0.57 | 4.16 | 0.0082 | 0.0159 | 0.0093 | 0.23 | 0.036 |
| 3037406 | 0.63 | 0.0022 | 1.3 | 0.63 | 0.0002 | 0.71 | 0.55 | 0.62 | 0.65 | 7.23 | 0.0004 | 0.0017 | 0.0021 | 0.02 | 0.004 |
| 3037950 | 0.61 | 0.0088 | 1.45 | 0.59 | 0.0138 | 0.53 | 0.64 | 0.61 | 0.57 | 3.82 | 0.0091 | 0.0095 | 0.0132 | 0.03 | 0.016 |
| 3039980 | 0.63 | 0.0011 | 1.37 | 0.64 | 0.0001 | 0.62 | 0.65 | 0.65 | 0.63 | 6.37 | 0.0001 | 0.0007 | 0.0009 | 0.15 | 0.016 |
| 3039983 | 0.6 | 0.0108 | 1.39 | 0.59 | 0.0138 | 0.58 | 0.59 | 0.6 | 0.58 | 5.64 | 0.0152 | 0.0074 | 0.006 | 0.44 | 0.07 |
| 3040116 | 0.64 | 0.001 | 1.43 | 0.64 | 0.0001 | 0.56 | 0.72 | 0.68 | 0.62 | 4.91 | 0 | 0.0011 | 0.0009 | 0.32 | 0.033 |
| 3041262 | 0.65 | 0.0004 | 1.43 | 0.63 | 0.0004 | 0.62 | 0.63 | 0.64 | 0.62 | 6.49 | 0.0003 | 0.0003 | 0.0003 | 0.06 | 0.003 |
| 3042004 | 0.62 | 0.005 | 1.35 | 0.62 | 0.0007 | 0.6 | 0.64 | 0.64 | 0.61 | 5.41 | 0.0007 | 0.0084 | 0.0067 | 0.29 | 0.039 |
| 3042424 | 0.63 | 0.0015 | 1.63 | 0.63 | 0.0002 | 0.63 | 0.63 | 0.64 | 0.63 | 5.85 | 0.0002 | 0.0012 | 0.001 | 0.06 | 0.004 |
| 3042426 | 0.62 | 0.0048 | 1.28 | 0.62 | 0.0011 | 0.61 | 0.62 | 0.63 | 0.61 | 6.11 | 0.0011 | 0.0035 | 0.0035 | 0.09 | 0.008 |
| 3042429 | 0.63 | 0.0017 | 1.27 | 0.59 | 0.0095 | 0.5 | 0.68 | 0.62 | 0.57 | 4.85 | 0.0061 | 0.0038 | 0.0043 | 0.32 | 0.123 |
| 3042430 | 0.64 | 0.0007 | 1.55 | 0.61 | 0.0028 | 0.62 | 0.59 | 0.61 | 0.6 | 5.62 | 0.0028 | 0.0009 | 0.0006 | 0.1 | 0.008 |
| 3042437 | 0.67 | 0.0001 | 1.35 | 0.66 | 0 | 0.64 | 0.67 | 0.67 | 0.65 | 6.81 | 0 | 0.0001 | 0.0001 | 0.01 | 0 |
| 3042453 | 0.64 | 0.0008 | 1.29 | 0.6 | 0.0064 | 0.49 | 0.71 | 0.64 | 0.57 | 3.84 | 0.0035 | 0.0021 | 0.0037 | 0.05 | 0.059 |
| 3048780 | 0.63 | 0.002 | 1.51 | 0.61 | 0.0018 | 0.62 | 0.6 | 0.62 | 0.61 | 4.61 | 0.0014 | 0.0025 | 0.0024 | 0.06 | 0.012 |
| 3048870 | 0.66 | 0.0001 | 1.36 | 0.62 | 0.0007 | 0.53 | 0.71 | 0.66 | 0.6 | 5.06 | 0.0003 | 0.0004 | 0.0002 | 0.01 | 0.002 |
| 3053384 | 0.68 | 0 | 1.58 | 0.64 | 0.0001 | 0.54 | 0.74 | 0.69 | 0.61 | 3.95 | 0 | 0 | 0 | 0.03 | 0.005 |
| 3056395 | 0.64 | 0.0005 | 1.27 | 0.64 | 0.0001 | 0.64 | 0.63 | 0.64 | 0.63 | 7.15 | 0.0001 | 0.0005 | 0.0006 | 0.05 | 0.011 |
| 3060052 | 0.64 | 0.0006 | 1.29 | 0.58 | 0.0276 | 0.44 | 0.72 | 0.62 | 0.55 | 3.77 | 0.0132 | 0.0022 | 0.0025 | 0.12 | 0.02 |
| 3061054 | 0.64 | 0.0007 | 1.26 | 0.62 | 0.0011 | 0.51 | 0.72 | 0.66 | 0.59 | 4.72 | 0.0005 | 0.0024 | 0.0014 | 0.14 | 0.011 |
| 3061144 | 0.62 | 0.0037 | 1.41 | 0.61 | 0.0028 | 0.52 | 0.69 | 0.64 | 0.59 | 4.89 | 0.002 | 0.0053 | 0.0065 | 0.07 | 0.027 |
| 3061758 | 0.64 | 0.0006 | 1.3 | 0.63 | 0.0002 | 0.58 | 0.68 | 0.66 | 0.61 | 5.26 | 0.0001 | 0.0011 | 0.0032 | 0.05 | 0.029 |
| 3062023 | 0.64 | 0.0006 | 1.26 | 0.64 | 0.0001 | 0.55 | 0.73 | 0.68 | 0.62 | 5.86 | 0 | 0.0002 | 0.0005 | 0.01 | 0.002 |
| 3064354 | 0.62 | 0.0028 | 1.3 | 0.63 | 0.0004 | 0.67 | 0.58 | 0.62 | 0.63 | 5.88 | 0.0005 | 0.003 | 0.002 | 0.07 | 0.006 |
| 3075645 | 0.62 | 0.0049 | 1.37 | 0.61 | 0.0028 | 0.64 | 0.57 | 0.61 | 0.61 | 5.92 | 0.0025 | 0.0062 | 0.006 | 0.16 | 0.027 |
| 3076355 | 0.67 | 0 | 1.49 | 0.61 | 0.0018 | 0.57 | 0.65 | 0.63 | 0.6 | 5.8 | 0.0019 | 0.0001 | 0 | 0.02 | 0.001 |
| 3076393 | 0.63 | 0.001 | 1.34 | 0.62 | 0.0007 | 0.54 | 0.7 | 0.65 | 0.6 | 4.19 | 0 | 0.0001 | 0.0009 | 0.01 | 0.038 |
| 3076490 | 0.63 | 0.0023 | 1.3 | 0.63 | 0.0004 | 0.61 | 0.64 | 0.64 | 0.62 | 6.59 | 0.0006 | 0.0037 | 0.0015 | 0.03 | 0.002 |
| 3081625 | 0.61 | 0.0064 | 1.26 | 0.59 | 0.0095 | 0.53 | 0.65 | 0.61 | 0.58 | 4.98 | 0.0063 | 0.0072 | 0.006 | 0.34 | 0.057 |
| 3087828 | 0.64 | 0.001 | 1.26 | 0.6 | 0.0064 | 0.5 | 0.69 | 0.63 | 0.58 | 5.49 | 0.0048 | 0.0012 | 0.0014 | 0.07 | 0.005 |
| 3087930 | 0.63 | 0.0022 | 1.36 | 0.62 | 0.0011 | 0.55 | 0.68 | 0.64 | 0.6 | 5.84 | 0.0022 | 0.0047 | 0.0026 | 0.09 | 0.014 |
| 3088570 | 0.6 | 0.0133 | 1.27 | 0.6 | 0.0064 | 0.63 | 0.56 | 0.6 | 0.6 | 5.28 | 0.0096 | 0.022 | 0.0139 | 0.1 | 0.013 |
| 3092505 | 0.61 | 0.0051 | 1.39 | 0.62 | 0.0007 | 0.65 | 0.59 | 0.62 | 0.62 | 5.8 | 0.0008 | 0.0048 | 0.0039 | 0.15 | 0.035 |
| 3094309 | 0.62 | 0.0029 | 1.57 | 0.58 | 0.0197 | 0.58 | 0.58 | 0.59 | 0.58 | 4.85 | 0.03 | 0.0054 | 0.0035 | 0.12 | 0.012 |
| 3096162 | 0.64 | 0.0005 | 1.31 | 0.62 | 0.0011 | 0.54 | 0.69 | 0.65 | 0.6 | 5.09 | 0.0022 | 0.0015 | 0.0008 | 0.04 | 0.005 |
| 3096409 | 0.63 | 0.0012 | 1.47 | 0.64 | 0.0001 | 0.5 | 0.79 | 0.71 | 0.61 | 4.9 | 0 | 0.0013 | 0.0016 | 0.01 | 0.003 |
| 3096979 | 0.66 | 0.0001 | 1.31 | 0.64 | 0.0001 | 0.6 | 0.68 | 0.66 | 0.63 | 5.57 | 0.0001 | 0.0002 | 0.0001 | 0.13 | 0.01 |
| 3099780 | 0.62 | 0.005 | 1.31 | 0.6 | 0.0042 | 0.6 | 0.6 | 0.61 | 0.6 | 5.8 | 0.0072 | 0.0074 | 0.0052 | 0.08 | 0.021 |
| 3100230 | 0.65 | 0.0004 | 1.35 | 0.63 | 0.0004 | 0.6 | 0.65 | 0.64 | 0.62 | 5.98 | 0.0006 | 0.0009 | 0.0007 | 0.05 | 0.009 |
| 3107638 | 0.61 | 0.0092 | 1.26 | 0.57 | 0.038 | 0.44 | 0.71 | 0.61 | 0.55 | 4.45 | 0.0104 | 0.0052 | 0.0122 | 0.24 | 0.085 |
| 3108472 | 0.65 | 0.0003 | 1.29 | 0.63 | 0.0004 | 0.59 | 0.66 | 0.65 | 0.61 | 6.54 | 0.0004 | 0.0004 | 0.0004 | 0.02 | 0.003 |
| 3108501 | 0.65 | 0.0003 | 1.27 | 0.61 | 0.0028 | 0.5 | 0.71 | 0.65 | 0.58 | 4.39 | 0.0047 | 0.0031 | 0.0017 | 0.19 | 0.031 |
| 3108938 | 0.61 | 0.0062 | 1.26 | 0.59 | 0.0138 | 0.55 | 0.62 | 0.6 | 0.58 | 4.59 | 0.0103 | 0.0101 | 0.0126 | 0.33 | 0.117 |
| 3111090 | 0.64 | 0.0008 | 1.28 | 0.64 | 0.0001 | 0.57 | 0.71 | 0.67 | 0.62 | 5.61 | 0 | 0.0003 | 0.0008 | 0.06 | 0.016 |
| 3111116 | 0.61 | 0.0073 | 1.37 | 0.61 | 0.0018 | 0.57 | 0.65 | 0.63 | 0.6 | 5.39 | 0.0008 | 0.0037 | 0.0051 | 0.22 | 0.042 |
| 3112517 | 0.63 | 0.0011 | 1.37 | 0.61 | 0.0018 | 0.58 | 0.64 | 0.63 | 0.6 | 6.28 | 0.0018 | 0.0009 | 0.0009 | 0.05 | 0.004 |
| 3114370 | 0.61 | 0.0054 | 1.27 | 0.58 | 0.0276 | 0.45 | 0.71 | 0.62 | 0.56 | 4.57 | 0.0231 | 0.0084 | 0.0093 | 0.46 | 0.124 |
| 3114664 | 0.6 | 0.0106 | 1.28 | 0.58 | 0.0197 | 0.53 | 0.63 | 0.6 | 0.57 | 5.06 | 0.0149 | 0.0158 | 0.0109 | 0.12 | 0.036 |
| 3124536 | 0.63 | 0.0021 | 1.28 | 0.63 | 0.0002 | 0.64 | 0.62 | 0.64 | 0.63 | 6.2 | 0.0004 | 0.0027 | 0.0023 | 0.01 | 0.002 |
| 3126094 | 0.63 | 0.002 | 1.31 | 0.64 | 0.0001 | 0.71 | 0.56 | 0.63 | 0.65 | 7.39 | 0.0002 | 0.0023 | 0.0012 | 0.06 | 0.006 |
| 3128680 | 0.61 | 0.008 | 1.35 | 0.57 | 0.038 | 0.54 | 0.6 | 0.59 | 0.56 | 5.38 | 0.0588 | 0.0095 | 0.0093 | 0.31 | 0.101 |
| 3129955 | 0.64 | 0.0007 | 1.3 | 0.62 | 0.0011 | 0.58 | 0.65 | 0.63 | 0.6 | 5.98 | 0.001 | 0.0015 | 0.0013 | 0.06 | 0.006 |
| 3131973 | 0.65 | 0.0003 | 1.31 | 0.64 | 0.0001 | 0.56 | 0.71 | 0.67 | 0.61 | 6.31 | 0.0001 | 0.0008 | 0.0006 | 0.07 | 0.006 |
| 3132336 | 0.61 | 0.0066 | 1.37 | 0.61 | 0.0018 | 0.53 | 0.69 | 0.64 | 0.59 | 4.42 | 0.0017 | 0.0249 | 0.0137 | 0.03 | 0.003 |
| 3133528 | 0.63 | 0.0014 | 1.3 | 0.62 | 0.0011 | 0.5 | 0.74 | 0.67 | 0.59 | 4.39 | 0.0004 | 0.001 | 0.0018 | 0.03 | 0.006 |
| 3134184 | 0.62 | 0.0035 | 1.27 | 0.57 | 0.038 | 0.39 | 0.77 | 0.63 | 0.55 | 3.84 | 0.0303 | 0.0046 | 0.0058 | 0.72 | 0.365 |
| 3136352 | 0.64 | 0.0009 | 1.31 | 0.62 | 0.0011 | 0.56 | 0.67 | 0.64 | 0.6 | 7.14 | 0.0007 | 0.0008 | 0.0013 | 0.03 | 0.006 |
| 3137586 | 0.63 | 0.0016 | 1.27 | 0.61 | 0.0028 | 0.62 | 0.59 | 0.61 | 0.6 | 6.36 | 0.0059 | 0.0023 | 0.0015 | 0.1 | 0.011 |
| 3138886 | 0.62 | 0.0028 | 1.25 | 0.58 | 0.0276 | 0.52 | 0.63 | 0.6 | 0.56 | 5.47 | 0.0263 | 0.0025 | 0.0028 | 0.14 | 0.023 |
| 3138980 | 0.6 | 0.0114 | 1.27 | 0.61 | 0.0028 | 0.6 | 0.61 | 0.62 | 0.6 | 5.6 | 0.0026 | 0.0142 | 0.0085 | 0.15 | 0.023 |
| 3139053 | 0.6 | 0.0142 | 1.28 | 0.62 | 0.0011 | 0.54 | 0.69 | 0.65 | 0.6 | 5.19 | 0.0009 | 0.0174 | 0.0166 | 0.44 | 0.07 |
| 3139926 | 0.66 | 0.0001 | 1.36 | 0.64 | 0.0001 | 0.56 | 0.72 | 0.68 | 0.62 | 5.9 | 0 | 0.0001 | 0.0002 | 0.03 | 0.014 |
| 3141863 | 0.62 | 0.0029 | 1.62 | 0.6 | 0.0042 | 0.54 | 0.66 | 0.63 | 0.59 | 3.89 | 0.0051 | 0.0093 | 0.0067 | 0.1 | 0.014 |
| 3145023 | 0.61 | 0.007 | 1.25 | 0.59 | 0.0095 | 0.52 | 0.66 | 0.62 | 0.58 | 5.1 | 0.0132 | 0.0084 | 0.0063 | 0.34 | 0.117 |
| 3145956 | 0.63 | 0.0021 | 1.27 | 0.63 | 0.0002 | 0.68 | 0.58 | 0.63 | 0.64 | 8.34 | 0.0004 | 0.0061 | 0.0046 | 0.15 | 0.011 |
| 3145966 | 0.63 | 0.001 | 1.26 | 0.63 | 0.0004 | 0.6 | 0.65 | 0.64 | 0.62 | 5.71 | 0.0006 | 0.0014 | 0.0012 | 0.13 | 0.012 |
| 3146538 | 0.61 | 0.0052 | 1.25 | 0.58 | 0.0197 | 0.47 | 0.7 | 0.62 | 0.56 | 4.01 | 0.0178 | 0.0178 | 0.0184 | 0.13 | 0.03 |
| 3146566 | 0.62 | 0.0027 | 1.35 | 0.61 | 0.0028 | 0.5 | 0.71 | 0.65 | 0.58 | 5.22 | 0.0012 | 0.0022 | 0.0033 | 0.05 | 0.014 |
| 3146788 | 0.62 | 0.0027 | 1.29 | 0.62 | 0.0007 | 0.6 | 0.64 | 0.64 | 0.61 | 7.65 | 0.0006 | 0.0018 | 0.0019 | 0.2 | 0.036 |
| 3146799 | 0.64 | 0.0005 | 1.25 | 0.63 | 0.0002 | 0.68 | 0.58 | 0.63 | 0.64 | 8.6 | 0.0004 | 0.0006 | 0.0007 | 0.09 | 0.011 |
| 3147328 | 0.62 | 0.0042 | 1.34 | 0.61 | 0.0028 | 0.56 | 0.65 | 0.63 | 0.59 | 5.1 | 0.0013 | 0.0018 | 0.0041 | 0.05 | 0.019 |
| 3148628 | 0.62 | 0.0031 | 1.38 | 0.6 | 0.0042 | 0.49 | 0.72 | 0.64 | 0.58 | 4.19 | 0.0029 | 0.0055 | 0.006 | 0.14 | 0.024 |
| 3149768 | 0.61 | 0.0076 | 1.39 | 0.62 | 0.0011 | 0.6 | 0.63 | 0.63 | 0.61 | 6.2 | 0.0016 | 0.0083 | 0.0058 | 0.16 | 0.018 |
| 3149773 | 0.65 | 0.0004 | 1.35 | 0.62 | 0.0007 | 0.58 | 0.66 | 0.64 | 0.61 | 4.81 | 0.001 | 0.0006 | 0.0005 | 0.15 | 0.009 |
| 3149864 | 0.65 | 0.0004 | 1.51 | 0.65 | 0 | 0.66 | 0.63 | 0.65 | 0.65 | 5.25 | 0 | 0.0003 | 0.0003 | 0.04 | 0.006 |
| 3151480 | 0.63 | 0.0014 | 1.75 | 0.62 | 0.0007 | 0.55 | 0.69 | 0.65 | 0.6 | 5.21 | 0.0004 | 0.0007 | 0.0006 | 0.04 | 0.005 |
| 3152560 | 0.61 | 0.0077 | 1.26 | 0.6 | 0.0064 | 0.53 | 0.66 | 0.62 | 0.58 | 4.5 | 0.0084 | 0.0181 | 0.0075 | 0.64 | 0.1 |
| 3153532 | 0.6 | 0.012 | 1.32 | 0.6 | 0.0042 | 0.53 | 0.67 | 0.63 | 0.58 | 4.96 | 0.0059 | 0.0179 | 0.0145 | 0.15 | 0.045 |
| 3157388 | 0.61 | 0.0102 | 1.43 | 0.6 | 0.0042 | 0.63 | 0.57 | 0.6 | 0.6 | 6.08 | 0.0055 | 0.0093 | 0.0074 | 0.18 | 0.024 |
| 3157461 | 0.61 | 0.0058 | 1.46 | 0.61 | 0.0028 | 0.66 | 0.55 | 0.6 | 0.61 | 5.23 | 0.0022 | 0.0053 | 0.0047 | 0.04 | 0.008 |
| 3157723 | 0.62 | 0.0037 | 1.34 | 0.62 | 0.0007 | 0.65 | 0.59 | 0.62 | 0.62 | 5.9 | 0.0006 | 0.0025 | 0.0025 | 0.1 | 0.028 |
| 3157847 | 0.63 | 0.0011 | 1.4 | 0.6 | 0.0042 | 0.53 | 0.67 | 0.63 | 0.58 | 5.48 | 0.0085 | 0.0016 | 0.0009 | 0.19 | 0.021 |
| 3164299 | 0.63 | 0.0019 | 1.3 | 0.63 | 0.0004 | 0.55 | 0.7 | 0.66 | 0.61 | 4.81 | 0.0002 | 0.0028 | 0.0036 | 0.18 | 0.061 |
| 3165138 | 0.64 | 0.0006 | 1.41 | 0.63 | 0.0004 | 0.59 | 0.66 | 0.65 | 0.61 | 4.24 | 0.0001 | 0.0013 | 0.0015 | 0.03 | 0.015 |
| 3165799 | 0.65 | 0.0004 | 1.44 | 0.61 | 0.0028 | 0.52 | 0.69 | 0.64 | 0.59 | 5.26 | 0.0015 | 0.0005 | 0.0004 | 0.03 | 0.002 |
| 3166029 | 0.63 | 0.0011 | 0.79 | 0.39 | 0.9996 | 0.41 | 0.38 | 0.4 | 0.38 | 4.13 | 0.0029 | 0.0021 | 0.0016 | 0.01 | 0.007 |
| 3166735 | 0.63 | 0.0015 | 1.47 | 0.62 | 0.0011 | 0.63 | 0.6 | 0.62 | 0.61 | 5.52 | 0.0011 | 0.0037 | 0.0025 | 0.07 | 0.013 |
| 3167990 | 0.64 | 0.0008 | 1.5 | 0.62 | 0.0011 | 0.54 | 0.69 | 0.65 | 0.6 | 6.06 | 0.0016 | 0.0009 | 0.0007 | 0.1 | 0.012 |
| 3168127 | 0.61 | 0.0096 | 1.25 | 0.6 | 0.0064 | 0.48 | 0.72 | 0.64 | 0.57 | 3.78 | 0.0076 | 0.0273 | 0.0127 | 0.65 | 0.149 |
| 3169011 | 0.71 | 0 | 0.79 | 0.35 | 1 | 0.24 | 0.47 | 0.32 | 0.37 | 3.13 | 0 | 0 | 0 | 0 | 0 |
| 3174268 | 0.67 | 0.0001 | 1.26 | 0.59 | 0.0095 | 0.47 | 0.72 | 0.64 | 0.57 | 5.25 | 0.0034 | 0.0001 | 0.0002 | 0.01 | 0.003 |
| 3174441 | 0.62 | 0.0043 | 1.29 | 0.59 | 0.0095 | 0.5 | 0.68 | 0.62 | 0.57 | 5.43 | 0.0199 | 0.0101 | 0.0054 | 0.34 | 0.023 |
| 3175632 | 0.65 | 0.0004 | 1.47 | 0.62 | 0.0011 | 0.64 | 0.59 | 0.62 | 0.62 | 5.06 | 0.0011 | 0.0003 | 0.0005 | 0.05 | 0.01 |
| 3175657 | 0.61 | 0.0094 | 1.28 | 0.58 | 0.0276 | 0.52 | 0.63 | 0.6 | 0.56 | 5.56 | 0.0345 | 0.0166 | 0.0136 | 0.56 | 0.149 |
| 3178635 | 0.63 | 0.001 | 1.43 | 0.59 | 0.0095 | 0.5 | 0.68 | 0.62 | 0.57 | 4.19 | 0.0078 | 0.0027 | 0.0032 | 0.06 | 0.019 |
| 3183178 | 0.6 | 0.0107 | 1.41 | 0.59 | 0.0095 | 0.53 | 0.65 | 0.61 | 0.58 | 4.45 | 0.0073 | 0.0055 | 0.0089 | 0.03 | 0.013 |
| 3190449 | 0.62 | 0.0043 | 1.32 | 0.59 | 0.0138 | 0.58 | 0.59 | 0.6 | 0.58 | 6.01 | 0.0129 | 0.0034 | 0.0056 | 0.09 | 0.038 |
| 3192674 | 0.6 | 0.0111 | 1.28 | 0.59 | 0.0138 | 0.52 | 0.65 | 0.61 | 0.57 | 4.88 | 0.0094 | 0.0146 | 0.0198 | 0.13 | 0.081 |
| 3197644 | 0.63 | 0.0013 | 1.39 | 0.58 | 0.0276 | 0.62 | 0.53 | 0.58 | 0.58 | 5.72 | 0.0406 | 0.0017 | 0.0014 | 0.03 | 0.006 |
| 3199457 | 0.63 | 0.0017 | 1.47 | 0.62 | 0.0007 | 0.55 | 0.69 | 0.65 | 0.6 | 4.76 | 0.0011 | 0.0025 | 0.0016 | 0.11 | 0.012 |
| 3199485 | 0.61 | 0.0069 | 1.29 | 0.61 | 0.0018 | 0.48 | 0.76 | 0.67 | 0.58 | 4.41 | 0.0008 | 0.0045 | 0.0045 | 0.18 | 0.048 |
| 3200638 | 0.62 | 0.0039 | 1.44 | 0.6 | 0.0042 | 0.56 | 0.64 | 0.62 | 0.59 | 4.43 | 0.004 | 0.0037 | 0.0028 | 0.76 | 0.074 |
| 3200711 | 0.61 | 0.0066 | 1.25 | 0.6 | 0.0064 | 0.51 | 0.68 | 0.63 | 0.58 | 5.69 | 0.0046 | 0.0081 | 0.0054 | 0.35 | 0.043 |
| 3200718 | 0.65 | 0.0004 | 1.28 | 0.59 | 0.0138 | 0.51 | 0.66 | 0.61 | 0.57 | 6.59 | 0.017 | 0.0004 | 0.0005 | 0.03 | 0.001 |
| 3200725 | 0.65 | 0.0002 | 1.35 | 0.62 | 0.0007 | 0.55 | 0.69 | 0.65 | 0.6 | 7.06 | 0.0007 | 0.0001 | 0.0002 | 0.04 | 0.004 |
| 3203822 | 0.62 | 0.0043 | 1.31 | 0.6 | 0.0042 | 0.61 | 0.59 | 0.61 | 0.6 | 4.86 | 0.0039 | 0.0058 | 0.0048 | 0.12 | 0.02 |
| 3205039 | 0.65 | 0.0004 | 1.26 | 0.63 | 0.0004 | 0.58 | 0.67 | 0.65 | 0.61 | 5.83 | 0.0004 | 0.0006 | 0.0007 | 0.02 | 0.004 |
| 3205424 | 0.62 | 0.0031 | 1.34 | 0.62 | 0.0011 | 0.52 | 0.71 | 0.65 | 0.59 | 4.24 | 0.0007 | 0.0068 | 0.0044 | 0.19 | 0.041 |
| 3212146 | 0.61 | 0.0078 | 1.36 | 0.62 | 0.0007 | 0.57 | 0.67 | 0.64 | 0.61 | 5.88 | 0.0007 | 0.0063 | 0.005 | 0.12 | 0.012 |
| 3212163 | 0.62 | 0.0032 | 1.38 | 0.62 | 0.0011 | 0.59 | 0.64 | 0.63 | 0.61 | 4.68 | 0.0009 | 0.0016 | 0.0024 | 0.02 | 0.008 |
| 3214671 | 0.63 | 0.0011 | 1.32 | 0.63 | 0.0004 | 0.63 | 0.62 | 0.63 | 0.62 | 4.48 | 0.0007 | 0.0013 | 0.0018 | 0.04 | 0.01 |
| 3214699 | 0.63 | 0.0015 | 1.31 | 0.63 | 0.0002 | 0.54 | 0.72 | 0.67 | 0.61 | 4.42 | 0.0001 | 0.003 | 0.0055 | 0.02 | 0.017 |
| 3219683 | 0.62 | 0.0032 | 1.33 | 0.6 | 0.0042 | 0.54 | 0.66 | 0.63 | 0.59 | 4.95 | 0.0036 | 0.0051 | 0.0057 | 0.25 | 0.056 |
| 3219711 | 0.67 | 0 | 1.55 | 0.6 | 0.0042 | 0.47 | 0.74 | 0.65 | 0.57 | 3.71 | 0.0008 | 0 | 0.0001 | 0 | 0 |
| 3219891 | 0.63 | 0.0016 | 1.53 | 0.61 | 0.0028 | 0.58 | 0.63 | 0.62 | 0.6 | 5.22 | 0.0021 | 0.0012 | 0.0017 | 0.03 | 0.007 |
| 3220159 | 0.62 | 0.0041 | 1.32 | 0.61 | 0.0018 | 0.64 | 0.58 | 0.61 | 0.61 | 7.14 | 0.0025 | 0.0055 | 0.0033 | 0.07 | 0.006 |
| 3222054 | 0.64 | 0.0008 | 1.3 | 0.62 | 0.0011 | 0.49 | 0.76 | 0.67 | 0.59 | 5.58 | 0.0004 | 0.0009 | 0.001 | 0.04 | 0.013 |
| 3222984 | 0.61 | 0.0078 | 1.26 | 0.54 | 0.2178 | 0.38 | 0.7 | 0.57 | 0.52 | 4.6 | 0.2985 | 0.0144 | 0.0113 | 0.32 | 0.046 |
| 3223876 | 0.63 | 0.0021 | 1.29 | 0.62 | 0.0007 | 0.57 | 0.67 | 0.64 | 0.61 | 5 | 0.0003 | 0.0015 | 0.0018 | 0.02 | 0.003 |
| 3225580 | 0.62 | 0.0045 | 1.26 | 0.58 | 0.0276 | 0.47 | 0.69 | 0.61 | 0.56 | 3.59 | 0.0542 | 0.0619 | 0.0186 | 0.25 | 0.029 |
| 3226470 | 0.63 | 0.0013 | 1.33 | 0.61 | 0.0028 | 0.51 | 0.7 | 0.64 | 0.58 | 3.52 | 0.0009 | 0.001 | 0.0024 | 0.01 | 0.004 |
| 3227646 | 0.63 | 0.0016 | 1.29 | 0.6 | 0.0042 | 0.59 | 0.61 | 0.61 | 0.59 | 5.96 | 0.0031 | 0.0022 | 0.0017 | 0.02 | 0.005 |
| 3233387 | 0.64 | 0.0005 | 1.56 | 0.65 | 0 | 0.66 | 0.64 | 0.66 | 0.65 | 5.55 | 0 | 0.0008 | 0.0003 | 0.07 | 0.004 |
| 3234175 | 0.61 | 0.007 | 1.33 | 0.61 | 0.0018 | 0.66 | 0.56 | 0.61 | 0.62 | 6.78 | 0.0022 | 0.0055 | 0.0052 | 0.1 | 0.021 |
| 3235500 | 0.63 | 0.0019 | 1.27 | 0.6 | 0.0042 | 0.55 | 0.65 | 0.62 | 0.59 | 4.55 | 0.0084 | 0.0074 | 0.0038 | 0.09 | 0.007 |
| 3237000 | 0.61 | 0.0092 | 1.34 | 0.59 | 0.0095 | 0.63 | 0.55 | 0.59 | 0.59 | 6.38 | 0.0102 | 0.015 | 0.01 | 0.3 | 0.041 |
| 3237001 | 0.61 | 0.0052 | 1.32 | 0.6 | 0.0042 | 0.65 | 0.55 | 0.6 | 0.61 | 7.77 | 0.0072 | 0.0096 | 0.0077 | 0.23 | 0.021 |
| 3239927 | 0.61 | 0.0054 | 1.25 | 0.6 | 0.0064 | 0.58 | 0.61 | 0.61 | 0.59 | 4.62 | 0.0068 | 0.0067 | 0.0058 | 0.06 | 0.017 |
| 3239982 | 0.64 | 0.0007 | 1.28 | 0.6 | 0.0042 | 0.51 | 0.69 | 0.63 | 0.58 | 6.06 | 0.0028 | 0.0016 | 0.0012 | 0.07 | 0.006 |
| 3240135 | 0.63 | 0.0018 | 1.28 | 0.59 | 0.0095 | 0.47 | 0.72 | 0.64 | 0.57 | 4.18 | 0.004 | 0.0057 | 0.005 | 0.08 | 0.022 |
| 3240409 | 0.66 | 0.0001 | 1.27 | 0.64 | 0.0001 | 0.57 | 0.71 | 0.67 | 0.62 | 7.78 | 0.0001 | 0.0002 | 0.0002 | 0.02 | 0.001 |
| 3240425 | 0.64 | 0.0005 | 1.29 | 0.61 | 0.0018 | 0.68 | 0.54 | 0.61 | 0.62 | 6.52 | 0.0018 | 0.0017 | 0.0011 | 0.06 | 0.006 |
| 3241499 | 0.63 | 0.0021 | 1.29 | 0.59 | 0.0095 | 0.5 | 0.69 | 0.63 | 0.57 | 4.32 | 0.0037 | 0.0026 | 0.0036 | 0.03 | 0.008 |
| 3241506 | 0.63 | 0.0011 | 1.3 | 0.62 | 0.0007 | 0.55 | 0.69 | 0.65 | 0.6 | 5.12 | 0.0003 | 0.0012 | 0.0012 | 0.09 | 0.029 |
| 3243292 | 0.65 | 0.0003 | 1.4 | 0.63 | 0.0004 | 0.57 | 0.68 | 0.65 | 0.61 | 5.85 | 0.0003 | 0.0003 | 0.0002 | 0.01 | 0.001 |
| 3250201 | 0.6 | 0.0125 | 1.57 | 0.59 | 0.0095 | 0.57 | 0.61 | 0.6 | 0.58 | 4.58 | 0.009 | 0.0224 | 0.0123 | 0.37 | 0.035 |
| 3251409 | 0.62 | 0.0042 | 1.28 | 0.62 | 0.0007 | 0.51 | 0.73 | 0.67 | 0.6 | 5.7 | 0.0003 | 0.001 | 0.0025 | 0.04 | 0.005 |
| 3252608 | 0.62 | 0.0037 | 1.29 | 0.61 | 0.0018 | 0.56 | 0.66 | 0.63 | 0.6 | 4.94 | 0.0012 | 0.0025 | 0.0036 | 0.12 | 0.094 |
| 3253445 | 0.65 | 0.0002 | 1.32 | 0.63 | 0.0002 | 0.65 | 0.61 | 0.63 | 0.63 | 7.12 | 0.0003 | 0.0003 | 0.0003 | 0.11 | 0.015 |
| 3253863 | 0.62 | 0.0043 | 1.28 | 0.59 | 0.0138 | 0.51 | 0.66 | 0.61 | 0.57 | 5.05 | 0.0232 | 0.0042 | 0.0032 | 0.14 | 0.02 |
| 3255269 | 0.61 | 0.0092 | 1.27 | 0.62 | 0.0007 | 0.57 | 0.67 | 0.64 | 0.61 | 6.42 | 0.0005 | 0.0081 | 0.007 | 0.25 | 0.021 |
| 3257404 | 0.6 | 0.0115 | 1.33 | 0.54 | 0.2178 | 0.38 | 0.7 | 0.57 | 0.52 | 4.85 | 0.2778 | 0.133 | 0.0798 | 0.19 | 0.03 |
| 3258021 | 0.65 | 0.0002 | 1.45 | 0.65 | 0 | 0.65 | 0.65 | 0.66 | 0.65 | 5.48 | 0 | 0.0005 | 0.0003 | 0.06 | 0.005 |
| 3259678 | 0.61 | 0.0092 | 1.35 | 0.59 | 0.0138 | 0.49 | 0.69 | 0.62 | 0.57 | 5.5 | 0.0074 | 0.0044 | 0.0071 | 0.1 | 0.029 |
| 3261550 | 0.62 | 0.0026 | 1.39 | 0.61 | 0.0028 | 0.5 | 0.71 | 0.65 | 0.58 | 4.53 | 0.0018 | 0.0046 | 0.0044 | 0.13 | 0.038 |
| 3262470 | 0.64 | 0.0006 | 1.27 | 0.59 | 0.0095 | 0.54 | 0.64 | 0.61 | 0.58 | 5.26 | 0.0133 | 0.0011 | 0.0012 | 0.11 | 0.028 |
| 3263804 | 0.63 | 0.0012 | 1.27 | 0.6 | 0.0064 | 0.55 | 0.64 | 0.62 | 0.58 | 5.78 | 0.0024 | 0.0014 | 0.0018 | 0.04 | 0.005 |
| 3264697 | 0.64 | 0.0007 | 1.31 | 0.62 | 0.0011 | 0.52 | 0.71 | 0.65 | 0.59 | 5.76 | 0.0009 | 0.0014 | 0.001 | 0.07 | 0.009 |
| 3264726 | 0.62 | 0.0024 | 1.59 | 0.59 | 0.0138 | 0.58 | 0.59 | 0.6 | 0.58 | 5.41 | 0.0154 | 0.0029 | 0.0019 | 0.15 | 0.017 |
| 3275175 | 0.63 | 0.0016 | 1.29 | 0.61 | 0.0028 | 0.54 | 0.67 | 0.63 | 0.59 | 5.15 | 0.0039 | 0.0025 | 0.002 | 0.12 | 0.013 |
| 3276455 | 0.62 | 0.0049 | 1.26 | 0.56 | 0.0681 | 0.43 | 0.7 | 0.6 | 0.54 | 3.84 | 0.0363 | 0.0135 | 0.0157 | 0.07 | 0.079 |
| 3277709 | 0.66 | 0.0001 | 1.33 | 0.66 | 0 | 0.58 | 0.73 | 0.69 | 0.63 | 4.68 | 0 | 0.0005 | 0.0004 | 0.06 | 0.009 |
| 3279906 | 0.62 | 0.0026 | 1.26 | 0.57 | 0.038 | 0.59 | 0.55 | 0.58 | 0.57 | 7.48 | 0.0556 | 0.0034 | 0.0028 | 0.08 | 0.009 |
| 3280961 | 0.62 | 0.0042 | 1.35 | 0.59 | 0.0138 | 0.48 | 0.7 | 0.62 | 0.57 | 4.37 | 0.0237 | 0.0174 | 0.0068 | 0.22 | 0.042 |
| 3282022 | 0.61 | 0.0058 | 1.4 | 0.58 | 0.0197 | 0.55 | 0.61 | 0.6 | 0.57 | 4.5 | 0.0225 | 0.0106 | 0.0065 | 0.34 | 0.048 |
| 3282170 | 0.63 | 0.0015 | 1.28 | 0.62 | 0.0011 | 0.57 | 0.66 | 0.64 | 0.6 | 3.58 | 0.0004 | 0.0032 | 0.0047 | 0.04 | 0.013 |
| 3282215 | 0.61 | 0.0067 | 1.49 | 0.59 | 0.0138 | 0.54 | 0.63 | 0.6 | 0.57 | 4.73 | 0.0203 | 0.0096 | 0.0069 | 0.07 | 0.006 |
| 3282220 | 0.63 | 0.0013 | 1.51 | 0.63 | 0.0002 | 0.59 | 0.67 | 0.65 | 0.62 | 5.09 | 0.0002 | 0.0016 | 0.001 | 0.03 | 0.003 |
| 3282254 | 0.62 | 0.0034 | 1.3 | 0.61 | 0.0028 | 0.6 | 0.61 | 0.62 | 0.6 | 5.13 | 0.0035 | 0.0036 | 0.0034 | 0.05 | 0.01 |
| 3284025 | 0.64 | 0.0009 | 1.33 | 0.63 | 0.0004 | 0.48 | 0.79 | 0.7 | 0.59 | 5.03 | 0.0001 | 0.0013 | 0.0011 | 0.05 | 0.01 |
| 3284217 | 0.64 | 0.0009 | 1.27 | 0.63 | 0.0002 | 0.69 | 0.57 | 0.63 | 0.64 | 6.48 | 0.0005 | 0.0008 | 0.0009 | 0.01 | 0.002 |
| 3284603 | 0.61 | 0.006 | 1.28 | 0.58 | 0.0197 | 0.53 | 0.63 | 0.6 | 0.57 | 5.47 | 0.0264 | 0.0095 | 0.007 | 0.15 | 0.026 |
| 3284665 | 0.62 | 0.0039 | 1.33 | 0.58 | 0.0276 | 0.54 | 0.61 | 0.59 | 0.57 | 5.97 | 0.0194 | 0.0033 | 0.0044 | 0.25 | 0.072 |
| 3294341 | 0.6 | 0.0107 | 1.39 | 0.6 | 0.0064 | 0.53 | 0.66 | 0.62 | 0.58 | 4.2 | 0.0055 | 0.0118 | 0.0091 | 0.08 | 0.012 |
| 3294501 | 0.63 | 0.0017 | 1.32 | 0.63 | 0.0002 | 0.61 | 0.65 | 0.65 | 0.62 | 4.97 | 0.0002 | 0.0032 | 0.0027 | 0.07 | 0.022 |
| 3298980 | 0.61 | 0.0095 | 1.34 | 0.62 | 0.0011 | 0.52 | 0.71 | 0.65 | 0.59 | 4.05 | 0.0018 | 0.03 | 0.0241 | 0.35 | 0.153 |
| 3301222 | 0.6 | 0.0138 | 1.33 | 0.6 | 0.0042 | 0.68 | 0.52 | 0.59 | 0.61 | 6.14 | 0.0063 | 0.0187 | 0.0112 | 0.29 | 0.034 |
| 3301703 | 0.6 | 0.014 | 1.34 | 0.61 | 0.0028 | 0.49 | 0.73 | 0.65 | 0.58 | 3.72 | 0.0038 | 0.0107 | 0.0074 | 0.32 | 0.041 |
| 3301858 | 0.6 | 0.0134 | 1.28 | 0.58 | 0.0276 | 0.39 | 0.78 | 0.64 | 0.55 | 4.07 | 0.0142 | 0.0254 | 0.0172 | 0.16 | 0.033 |
| 3301863 | 0.62 | 0.0033 | 1.36 | 0.63 | 0.0004 | 0.63 | 0.62 | 0.63 | 0.62 | 7.19 | 0.0008 | 0.0056 | 0.0027 | 0.22 | 0.012 |
| 3302047 | 0.67 | 0.0001 | 1.35 | 0.62 | 0.0011 | 0.53 | 0.7 | 0.65 | 0.59 | 5.12 | 0.0006 | 0.0001 | 0.0002 | 0.03 | 0.003 |
| 3304013 | 0.62 | 0.0031 | 1.29 | 0.61 | 0.0028 | 0.64 | 0.57 | 0.61 | 0.61 | 6.38 | 0.0046 | 0.0037 | 0.0025 | 0.1 | 0.005 |
| 3304626 | 0.61 | 0.0076 | 1.25 | 0.6 | 0.0042 | 0.5 | 0.7 | 0.64 | 0.58 | 5.73 | 0.0021 | 0.0052 | 0.0043 | 0.17 | 0.031 |
| 3304652 | 0.65 | 0.0003 | 1.38 | 0.6 | 0.0064 | 0.49 | 0.71 | 0.64 | 0.57 | 4.91 | 0.0031 | 0.0002 | 0.0004 | 0.09 | 0.011 |
| 3307940 | 0.61 | 0.0096 | 1.28 | 0.58 | 0.0276 | 0.5 | 0.65 | 0.6 | 0.56 | 3.98 | 0.0357 | 0.0117 | 0.009 | 0.15 | 0.03 |
| 3308532 | 0.62 | 0.0038 | 1.26 | 0.56 | 0.0889 | 0.4 | 0.72 | 0.6 | 0.54 | 5.32 | 0.033 | 0.0273 | 0.0286 | 0.23 | 0.05 |
| 3309267 | 0.6 | 0.0121 | 1.32 | 0.59 | 0.0138 | 0.57 | 0.6 | 0.6 | 0.58 | 5.15 | 0.006 | 0.0079 | 0.0119 | 0.12 | 0.043 |
| 3311304 | 0.65 | 0.0002 | 1.3 | 0.61 | 0.0028 | 0.45 | 0.78 | 0.67 | 0.58 | 3.87 | 0.0022 | 0.0017 | 0.0007 | 0.07 | 0.003 |
| 3317557 | 0.65 | 0.0002 | 1.35 | 0.57 | 0.0513 | 0.43 | 0.71 | 0.61 | 0.55 | 3.97 | 0.0488 | 0.0074 | 0.0035 | 0 | 0.001 |
| 3325714 | 0.61 | 0.0053 | 1.29 | 0.58 | 0.0197 | 0.47 | 0.7 | 0.62 | 0.56 | 4.46 | 0.0169 | 0.007 | 0.0057 | 0.27 | 0.036 |
| 3325963 | 0.64 | 0.0006 | 1.43 | 0.63 | 0.0004 | 0.65 | 0.6 | 0.63 | 0.63 | 5.49 | 0.0006 | 0.0017 | 0.0006 | 0.05 | 0.004 |
| 3326246 | 0.63 | 0.0022 | 1.26 | 0.59 | 0.0138 | 0.42 | 0.77 | 0.65 | 0.56 | 4.09 | 0.0088 | 0.0082 | 0.0071 | 0.08 | 0.035 |
| 3326465 | 0.66 | 0.0001 | 1.43 | 0.61 | 0.0028 | 0.54 | 0.67 | 0.63 | 0.59 | 3.43 | 0.0021 | 0.0001 | 0.0002 | 0.06 | 0.004 |
| 3329941 | 0.64 | 0.0008 | 1.3 | 0.62 | 0.0011 | 0.57 | 0.66 | 0.64 | 0.6 | 4.95 | 0.0005 | 0.0002 | 0.0008 | 0.03 | 0.013 |
| 3331532 | 0.66 | 0.0001 | 1.29 | 0.64 | 0.0001 | 0.6 | 0.68 | 0.66 | 0.63 | 6.28 | 0.0001 | 0.0001 | 0.0001 | 0.01 | 0 |
| 3331573 | 0.62 | 0.0025 | 1.43 | 0.63 | 0.0002 | 0.63 | 0.63 | 0.64 | 0.63 | 5.1 | 0.0002 | 0.0008 | 0.001 | 0.09 | 0.016 |
| 3331613 | 0.65 | 0.0002 | 1.26 | 0.63 | 0.0004 | 0.55 | 0.7 | 0.66 | 0.61 | 6.82 | 0.0004 | 0.0003 | 0.0002 | 0.01 | 0.001 |
| 3331614 | 0.64 | 0.0006 | 1.36 | 0.63 | 0.0004 | 0.72 | 0.53 | 0.61 | 0.65 | 6.9 | 0.0006 | 0.0014 | 0.0008 | 0.03 | 0.002 |
| 3333687 | 0.6 | 0.0109 | 1.29 | 0.59 | 0.0138 | 0.58 | 0.59 | 0.6 | 0.58 | 5.39 | 0.021 | 0.0263 | 0.0172 | 0.22 | 0.06 |
| 3334162 | 0.64 | 0.0007 | 1.31 | 0.61 | 0.0028 | 0.5 | 0.72 | 0.65 | 0.58 | 5.02 | 0.0008 | 0.0003 | 0.001 | 0.05 | 0.066 |
| 3334515 | 0.62 | 0.003 | 1.26 | 0.6 | 0.0042 | 0.67 | 0.53 | 0.6 | 0.61 | 7.13 | 0.0057 | 0.0025 | 0.0022 | 0.02 | 0.002 |
| 3335173 | 0.66 | 0.0001 | 1.47 | 0.62 | 0.0007 | 0.67 | 0.57 | 0.62 | 0.63 | 6.87 | 0.0011 | 0.0001 | 0.0001 | 0.02 | 0.002 |
| 3335176 | 0.65 | 0.0003 | 1.38 | 0.61 | 0.0028 | 0.6 | 0.61 | 0.62 | 0.6 | 6.73 | 0.0032 | 0.0003 | 0.0003 | 0.02 | 0.002 |
| 3335178 | 0.65 | 0.0003 | 1.31 | 0.62 | 0.0007 | 0.62 | 0.62 | 0.63 | 0.62 | 7 | 0.0004 | 0.0002 | 0.0003 | 0.01 | 0.002 |
| 3335179 | 0.65 | 0.0002 | 1.46 | 0.63 | 0.0002 | 0.6 | 0.66 | 0.65 | 0.62 | 5.84 | 0.0002 | 0.0002 | 0.0002 | 0.01 | 0.002 |
| 3335181 | 0.65 | 0.0002 | 1.29 | 0.61 | 0.0018 | 0.69 | 0.53 | 0.6 | 0.63 | 8.16 | 0.0027 | 0.0008 | 0.0008 | 0.03 | 0.003 |
| 3335182 | 0.64 | 0.0004 | 1.29 | 0.62 | 0.0007 | 0.67 | 0.57 | 0.62 | 0.63 | 8.91 | 0.001 | 0.0008 | 0.0009 | 0.03 | 0.003 |
| 3335187 | 0.65 | 0.0002 | 1.38 | 0.64 | 0.0001 | 0.69 | 0.59 | 0.64 | 0.65 | 8.55 | 0.0001 | 0.0004 | 0.0005 | 0.03 | 0.002 |
| 3335188 | 0.65 | 0.0003 | 1.27 | 0.63 | 0.0002 | 0.7 | 0.56 | 0.62 | 0.65 | 9.15 | 0.0004 | 0.0007 | 0.0005 | 0.03 | 0.003 |
| 3335189 | 0.62 | 0.0037 | 1.29 | 0.59 | 0.0138 | 0.59 | 0.58 | 0.59 | 0.58 | 5.87 | 0.0145 | 0.0018 | 0.0036 | 0.05 | 0.023 |
| 3335191 | 0.65 | 0.0004 | 1.32 | 0.62 | 0.0011 | 0.65 | 0.58 | 0.62 | 0.62 | 8.92 | 0.0014 | 0.0007 | 0.0012 | 0.04 | 0.005 |
| 3335231 | 0.64 | 0.0006 | 1.27 | 0.63 | 0.0004 | 0.75 | 0.5 | 0.61 | 0.66 | 7.56 | 0.0005 | 0.0008 | 0.0006 | 0.03 | 0.004 |
| 3337724 | 0.64 | 0.0006 | 1.26 | 0.64 | 0.0001 | 0.64 | 0.64 | 0.65 | 0.64 | 7.12 | 0.0001 | 0.0012 | 0.0005 | 0.09 | 0.003 |
| 3338618 | 0.65 | 0.0002 | 1.36 | 0.65 | 0 | 0.62 | 0.67 | 0.66 | 0.63 | 7.42 | 0 | 0.0003 | 0.0003 | 0.02 | 0.003 |
| 3341466 | 0.63 | 0.0017 | 1.25 | 0.63 | 0.0002 | 0.68 | 0.58 | 0.63 | 0.64 | 5.33 | 0.0002 | 0.0021 | 0.0027 | 0.09 | 0.033 |
| 3341504 | 0.64 | 0.0009 | 1.5 | 0.6 | 0.0042 | 0.58 | 0.62 | 0.61 | 0.59 | 5.8 | 0.0053 | 0.0011 | 0.0009 | 0.05 | 0.005 |
| 3344853 | 0.62 | 0.005 | 1.6 | 0.6 | 0.0042 | 0.63 | 0.57 | 0.6 | 0.6 | 4.8 | 0.0033 | 0.0048 | 0.0032 | 0.07 | 0.009 |
| 3344889 | 0.62 | 0.0049 | 1.34 | 0.59 | 0.0138 | 0.49 | 0.69 | 0.62 | 0.57 | 3.99 | 0.0097 | 0.0046 | 0.0044 | 0.11 | 0.017 |
| 3345453 | 0.61 | 0.0074 | 1.57 | 0.6 | 0.0064 | 0.55 | 0.64 | 0.62 | 0.58 | 5.32 | 0.0054 | 0.0092 | 0.0078 | 0.11 | 0.047 |
| 3347691 | 0.61 | 0.0072 | 1.26 | 0.59 | 0.0138 | 0.55 | 0.62 | 0.6 | 0.58 | 6.2 | 0.0155 | 0.0116 | 0.0059 | 0.14 | 0.02 |
| 3352533 | 0.67 | 0 | 1.25 | 0.63 | 0.0002 | 0.69 | 0.57 | 0.63 | 0.64 | 5.45 | 0.0005 | 0.0001 | 0.0001 | 0.02 | 0.001 |
| 3352561 | 0.62 | 0.0051 | 1.32 | 0.62 | 0.0007 | 0.6 | 0.64 | 0.64 | 0.61 | 4.61 | 0.0012 | 0.0063 | 0.005 | 0.15 | 0.027 |
| 3352585 | 0.61 | 0.0063 | 1.34 | 0.62 | 0.0011 | 0.7 | 0.53 | 0.61 | 0.63 | 5.69 | 0.0011 | 0.0066 | 0.0071 | 0.08 | 0.019 |
| 3352925 | 0.62 | 0.0027 | 1.32 | 0.62 | 0.0011 | 0.57 | 0.66 | 0.64 | 0.6 | 3.22 | 0.0009 | 0.0009 | 0.0012 | 0.04 | 0.004 |
| 3354966 | 0.62 | 0.003 | 1.29 | 0.62 | 0.0011 | 0.61 | 0.62 | 0.63 | 0.61 | 7.42 | 0.0013 | 0.0043 | 0.0041 | 0.1 | 0.013 |
| 3356173 | 0.62 | 0.0045 | 1.3 | 0.6 | 0.0042 | 0.56 | 0.64 | 0.62 | 0.59 | 4.84 | 0.0026 | 0.0039 | 0.0045 | 0.02 | 0.011 |
| 3358364 | 0.64 | 0.0004 | 1.4 | 0.63 | 0.0004 | 0.71 | 0.54 | 0.62 | 0.65 | 5.9 | 0.0004 | 0.0005 | 0.0003 | 0.02 | 0.002 |
| 3359382 | 0.64 | 0.0006 | 1.35 | 0.6 | 0.0042 | 0.49 | 0.72 | 0.64 | 0.58 | 4.53 | 0.0026 | 0.0004 | 0.0008 | 0.12 | 0.013 |
| 3362739 | 0.62 | 0.0023 | 1.42 | 0.6 | 0.0064 | 0.52 | 0.67 | 0.62 | 0.58 | 4.72 | 0.0043 | 0.0022 | 0.0026 | 0.01 | 0.01 |
| 3362742 | 0.65 | 0.0002 | 1.56 | 0.64 | 0.0001 | 0.66 | 0.62 | 0.64 | 0.64 | 5.09 | 0.0001 | 0.0003 | 0.0004 | 0.02 | 0.009 |
| 3362745 | 0.61 | 0.007 | 1.39 | 0.6 | 0.0042 | 0.56 | 0.64 | 0.62 | 0.59 | 5.36 | 0.0023 | 0.0051 | 0.0054 | 0.08 | 0.024 |
| 3362763 | 0.67 | 0 | 1.25 | 0.62 | 0.0007 | 0.54 | 0.7 | 0.65 | 0.6 | 5.41 | 0.0006 | 0.0002 | 0.0001 | 0.01 | 0.001 |
| 3362773 | 0.64 | 0.001 | 1.28 | 0.61 | 0.0018 | 0.55 | 0.67 | 0.64 | 0.59 | 7.19 | 0.0012 | 0.0013 | 0.0008 | 0.06 | 0.003 |
| 3362943 | 0.62 | 0.0049 | 1.47 | 0.61 | 0.0018 | 0.58 | 0.64 | 0.63 | 0.6 | 5.08 | 0.0014 | 0.0056 | 0.004 | 0.1 | 0.01 |
| 3363519 | 0.61 | 0.0099 | 1.31 | 0.63 | 0.0004 | 0.6 | 0.65 | 0.64 | 0.62 | 4.57 | 0 | 0.0093 | 0.017 | 0.03 | 0.065 |
| 3363981 | 0.6 | 0.0113 | 1.29 | 0.58 | 0.0276 | 0.47 | 0.69 | 0.61 | 0.56 | 3.66 | 0.0122 | 0.0376 | 0.043 | 0.14 | 0.055 |
| 3364729 | 0.61 | 0.0056 | 1.36 | 0.56 | 0.0681 | 0.49 | 0.64 | 0.58 | 0.55 | 4.73 | 0.0521 | 0.0049 | 0.0071 | 0.14 | 0.118 |
| 3364770 | 0.64 | 0.0006 | 1.28 | 0.6 | 0.0064 | 0.52 | 0.67 | 0.62 | 0.58 | 4.25 | 0.0074 | 0.0011 | 0.0008 | 0.06 | 0.012 |
| 3364800 | 0.64 | 0.0006 | 1.32 | 0.64 | 0.0001 | 0.58 | 0.69 | 0.66 | 0.62 | 3.64 | 0.0001 | 0.002 | 0.001 | 0.08 | 0.017 |
| 3368920 | 0.6 | 0.011 | 1.34 | 0.6 | 0.0064 | 0.55 | 0.64 | 0.62 | 0.58 | 5.71 | 0.0113 | 0.0142 | 0.0091 | 0.13 | 0.018 |
| 3371737 | 0.61 | 0.0063 | 1.25 | 0.61 | 0.0028 | 0.57 | 0.64 | 0.62 | 0.59 | 6.25 | 0.0034 | 0.0073 | 0.0052 | 0.13 | 0.018 |
| 3372461 | 0.61 | 0.0093 | 1.39 | 0.57 | 0.0513 | 0.47 | 0.67 | 0.59 | 0.55 | 3.62 | 0.0298 | 0.0243 | 0.0231 | 0.05 | 0.019 |
| 3375309 | 0.6 | 0.0134 | 1.54 | 0.6 | 0.0064 | 0.62 | 0.57 | 0.6 | 0.6 | 4.78 | 0.0149 | 0.0179 | 0.0088 | 0.18 | 0.018 |
| 3375752 | 0.66 | 0.0001 | 1.3 | 0.61 | 0.0018 | 0.6 | 0.62 | 0.62 | 0.6 | 5.1 | 0.0026 | 0.0006 | 0.0004 | 0.02 | 0.003 |
| 3375863 | 0.63 | 0.0016 | 1.3 | 0.61 | 0.0018 | 0.58 | 0.64 | 0.63 | 0.6 | 4.63 | 0.0048 | 0.0035 | 0.0017 | 0.04 | 0.005 |
| 3376090 | 0.64 | 0.0005 | 1.42 | 0.62 | 0.0007 | 0.64 | 0.6 | 0.63 | 0.62 | 5.66 | 0.0009 | 0.0004 | 0.0004 | 0.07 | 0.009 |
| 3376775 | 0.62 | 0.0036 | 1.25 | 0.59 | 0.0095 | 0.62 | 0.56 | 0.59 | 0.59 | 5.6 | 0.0068 | 0.0043 | 0.0049 | 0.09 | 0.021 |
| 3377457 | 0.61 | 0.0057 | 1.58 | 0.62 | 0.0007 | 0.62 | 0.62 | 0.63 | 0.62 | 4.79 | 0.0002 | 0.0042 | 0.0044 | 0.04 | 0.008 |
| 3377464 | 0.62 | 0.004 | 1.36 | 0.63 | 0.0004 | 0.65 | 0.6 | 0.63 | 0.63 | 6.05 | 0.0004 | 0.0034 | 0.0024 | 0.09 | 0.013 |
| 3377621 | 0.64 | 0.0005 | 1.42 | 0.62 | 0.0011 | 0.68 | 0.55 | 0.61 | 0.63 | 6.62 | 0.0015 | 0.0003 | 0.0004 | 0.03 | 0.007 |
| 3377623 | 0.63 | 0.0016 | 1.37 | 0.6 | 0.0064 | 0.64 | 0.55 | 0.6 | 0.6 | 6.65 | 0.0059 | 0.0009 | 0.0011 | 0.04 | 0.008 |
| 3377625 | 0.63 | 0.0017 | 1.31 | 0.61 | 0.0028 | 0.73 | 0.48 | 0.59 | 0.64 | 6.78 | 0.0033 | 0.0021 | 0.0019 | 0.1 | 0.01 |
| 3377632 | 0.64 | 0.001 | 1.37 | 0.59 | 0.0095 | 0.66 | 0.52 | 0.59 | 0.6 | 6.13 | 0.0099 | 0.0008 | 0.0009 | 0.03 | 0.004 |
| 3377633 | 0.61 | 0.0054 | 1.26 | 0.59 | 0.0095 | 0.53 | 0.65 | 0.61 | 0.58 | 6.83 | 0.0047 | 0.0024 | 0.005 | 0.08 | 0.038 |
| 3377659 | 0.65 | 0.0004 | 1.29 | 0.61 | 0.0018 | 0.65 | 0.57 | 0.61 | 0.62 | 9.15 | 0.0022 | 0.0009 | 0.0011 | 0.04 | 0.003 |
| 3377660 | 0.64 | 0.0006 | 1.29 | 0.63 | 0.0004 | 0.66 | 0.59 | 0.63 | 0.63 | 7.82 | 0.0004 | 0.0011 | 0.0013 | 0.04 | 0.004 |
| 3379122 | 0.62 | 0.0028 | 1.44 | 0.61 | 0.0018 | 0.67 | 0.55 | 0.61 | 0.62 | 6.31 | 0.0018 | 0.0035 | 0.0029 | 0.07 | 0.007 |
| 3379574 | 0.61 | 0.0069 | 1.36 | 0.6 | 0.0042 | 0.55 | 0.65 | 0.62 | 0.59 | 4.73 | 0.0024 | 0.0056 | 0.0057 | 0.13 | 0.019 |
| 3382981 | 0.66 | 0.0001 | 1.29 | 0.62 | 0.0011 | 0.6 | 0.63 | 0.63 | 0.61 | 5.13 | 0.0008 | 0.0002 | 0.0002 | 0.01 | 0.003 |
| 3382984 | 0.64 | 0.0004 | 1.32 | 0.6 | 0.0042 | 0.57 | 0.63 | 0.62 | 0.59 | 5.6 | 0.0036 | 0.0006 | 0.0007 | 0.05 | 0.011 |
| 3383148 | 0.63 | 0.0011 | 1.39 | 0.62 | 0.0007 | 0.69 | 0.55 | 0.61 | 0.64 | 6.75 | 0.0009 | 0.0017 | 0.0012 | 0.02 | 0.002 |
| 3383149 | 0.61 | 0.0056 | 1.47 | 0.58 | 0.0197 | 0.59 | 0.57 | 0.59 | 0.58 | 5.88 | 0.0217 | 0.0051 | 0.0047 | 0.09 | 0.013 |
| 3384489 | 0.64 | 0.0008 | 1.41 | 0.61 | 0.0018 | 0.57 | 0.65 | 0.63 | 0.6 | 5.19 | 0.0022 | 0.0009 | 0.0009 | 0.02 | 0.002 |
| 3384495 | 0.62 | 0.0035 | 1.35 | 0.63 | 0.0004 | 0.64 | 0.61 | 0.63 | 0.63 | 4.38 | 0.0011 | 0.0121 | 0.0058 | 0.41 | 0.051 |
| 3385045 | 0.65 | 0.0002 | 1.27 | 0.6 | 0.0042 | 0.43 | 0.79 | 0.67 | 0.57 | 3.72 | 0.0004 | 0.0002 | 0.0008 | 0.01 | 0.004 |
| 3385178 | 0.6 | 0.0107 | 1.34 | 0.6 | 0.0064 | 0.53 | 0.66 | 0.62 | 0.58 | 4.08 | 0.0055 | 0.0123 | 0.011 | 0.23 | 0.067 |
| 3386816 | 0.64 | 0.0009 | 1.33 | 0.61 | 0.0018 | 0.56 | 0.66 | 0.63 | 0.6 | 7.1 | 0.0036 | 0.0021 | 0.0009 | 0.06 | 0.003 |
| 3387176 | 0.61 | 0.009 | 1.26 | 0.59 | 0.0095 | 0.48 | 0.71 | 0.63 | 0.57 | 4.06 | 0.0173 | 0.0177 | 0.0116 | 0.48 | 0.068 |
| 3387436 | 0.64 | 0.0004 | 1.29 | 0.61 | 0.0028 | 0.54 | 0.67 | 0.63 | 0.59 | 5.92 | 0.0032 | 0.0011 | 0.0004 | 0.02 | 0.002 |
| 3388633 | 0.64 | 0.0005 | 1.31 | 0.65 | 0 | 0.58 | 0.72 | 0.69 | 0.63 | 5.36 | 0 | 0.0006 | 0.0005 | 0.03 | 0.006 |
| 3393789 | 0.62 | 0.0033 | 1.38 | 0.63 | 0.0004 | 0.61 | 0.64 | 0.64 | 0.62 | 5.27 | 0.0008 | 0.0028 | 0.0022 | 0.08 | 0.006 |
| 3394076 | 0.65 | 0.0002 | 1.28 | 0.64 | 0.0001 | 0.59 | 0.69 | 0.67 | 0.62 | 7.04 | 0.0001 | 0.0002 | 0.0003 | 0.04 | 0.003 |
| 3394529 | 0.6 | 0.0109 | 1.35 | 0.59 | 0.0138 | 0.53 | 0.64 | 0.61 | 0.57 | 4.77 | 0.0115 | 0.0238 | 0.0278 | 0.58 | 0.288 |
| 3395420 | 0.61 | 0.0057 | 1.37 | 0.61 | 0.0018 | 0.64 | 0.58 | 0.61 | 0.61 | 6.13 | 0.0033 | 0.0052 | 0.0057 | 0.11 | 0.016 |
| 3395427 | 0.62 | 0.0026 | 1.29 | 0.62 | 0.0007 | 0.59 | 0.65 | 0.64 | 0.61 | 5.82 | 0.0004 | 0.0026 | 0.0032 | 0.05 | 0.015 |
| 3400152 | 0.62 | 0.0024 | 1.26 | 0.6 | 0.0042 | 0.58 | 0.62 | 0.61 | 0.59 | 4.91 | 0.0069 | 0.0035 | 0.0021 | 0.16 | 0.022 |
| 3402624 | 0.61 | 0.0103 | 1.25 | 0.58 | 0.0276 | 0.51 | 0.64 | 0.6 | 0.56 | 5.13 | 0.0325 | 0.0107 | 0.0113 | 0.04 | 0.018 |
| 3402667 | 0.63 | 0.0021 | 1.25 | 0.59 | 0.0095 | 0.6 | 0.58 | 0.6 | 0.59 | 5.61 | 0.0058 | 0.0017 | 0.002 | 0.01 | 0.003 |
| 3402720 | 0.63 | 0.0012 | 1.4 | 0.61 | 0.0018 | 0.58 | 0.64 | 0.63 | 0.6 | 5.64 | 0.0021 | 0.0023 | 0.0015 | 0.04 | 0.004 |
| 3406062 | 0.63 | 0.0018 | 1.29 | 0.64 | 0.0001 | 0.57 | 0.7 | 0.67 | 0.62 | 4.22 | 0.0002 | 0.0021 | 0.002 | 0.06 | 0.005 |
| 3407177 | 0.64 | 0.0005 | 1.25 | 0.54 | 0.2178 | 0.34 | 0.74 | 0.58 | 0.52 | 3.85 | 0.1199 | 0.0007 | 0.0022 | 0.08 | 0.029 |
| 3407275 | 0.61 | 0.0068 | 1.28 | 0.56 | 0.0681 | 0.46 | 0.67 | 0.59 | 0.55 | 5.5 | 0.0872 | 0.0085 | 0.0089 | 0.38 | 0.1 |
| 3409292 | 0.61 | 0.0081 | 1.31 | 0.63 | 0.0002 | 0.59 | 0.67 | 0.65 | 0.62 | 5.99 | 0.0002 | 0.0054 | 0.0088 | 0.25 | 0.06 |
| 3412651 | 0.63 | 0.0015 | 1.31 | 0.6 | 0.0042 | 0.48 | 0.73 | 0.65 | 0.58 | 5.63 | 0.0043 | 0.0023 | 0.0018 | 0.03 | 0.005 |
| 3414226 | 0.64 | 0.0007 | 1.4 | 0.65 | 0 | 0.67 | 0.62 | 0.65 | 0.65 | 6.01 | 0 | 0.0006 | 0.0004 | 0.01 | 0.001 |
| 3416068 | 0.65 | 0.0003 | 1.34 | 0.62 | 0.0011 | 0.63 | 0.6 | 0.62 | 0.61 | 5.69 | 0.001 | 0.0002 | 0.0004 | 0.01 | 0.005 |
| 3416501 | 0.6 | 0.0141 | 1.26 | 0.6 | 0.0064 | 0.49 | 0.71 | 0.64 | 0.57 | 4.91 | 0.0061 | 0.025 | 0.02 | 0.24 | 0.076 |
| 3417160 | 0.64 | 0.0004 | 1.38 | 0.62 | 0.0011 | 0.61 | 0.62 | 0.63 | 0.61 | 6.47 | 0.0013 | 0.0004 | 0.0005 | 0.01 | 0.002 |
| 3417666 | 0.64 | 0.0005 | 1.31 | 0.58 | 0.0276 | 0.55 | 0.6 | 0.59 | 0.57 | 6.07 | 0.0331 | 0.0008 | 0.0006 | 0.02 | 0.003 |
| 3418209 | 0.62 | 0.0041 | 1.34 | 0.6 | 0.0064 | 0.55 | 0.64 | 0.62 | 0.58 | 4.18 | 0.006 | 0.0085 | 0.0083 | 0.06 | 0.018 |
| 3419262 | 0.63 | 0.0021 | 1.29 | 0.62 | 0.0007 | 0.54 | 0.7 | 0.65 | 0.6 | 5.76 | 0.0005 | 0.0031 | 0.0026 | 0.06 | 0.011 |
| 3419622 | 0.64 | 0.0009 | 1.35 | 0.62 | 0.0011 | 0.6 | 0.63 | 0.63 | 0.61 | 4.99 | 0.0005 | 0.0004 | 0.0007 | 0.04 | 0.024 |
| 3421154 | 0.62 | 0.0037 | 1.42 | 0.59 | 0.0095 | 0.65 | 0.53 | 0.59 | 0.6 | 5.74 | 0.0115 | 0.0039 | 0.0032 | 0.07 | 0.007 |
| 3421223 | 0.61 | 0.0062 | 1.58 | 0.61 | 0.0018 | 0.56 | 0.66 | 0.63 | 0.6 | 4.69 | 0.0017 | 0.0054 | 0.0035 | 0.09 | 0.015 |
| 3421350 | 0.63 | 0.0014 | 1.39 | 0.6 | 0.0064 | 0.57 | 0.62 | 0.61 | 0.59 | 5.56 | 0.0042 | 0.001 | 0.0009 | 0.04 | 0.009 |
| 3421492 | 0.61 | 0.006 | 1.25 | 0.6 | 0.0064 | 0.48 | 0.72 | 0.64 | 0.57 | 4.45 | 0.0054 | 0.0103 | 0.005 | 0.13 | 0.024 |
| 3421652 | 0.61 | 0.0085 | 1.31 | 0.61 | 0.0028 | 0.62 | 0.59 | 0.61 | 0.6 | 6.32 | 0.0035 | 0.0138 | 0.0078 | 0.2 | 0.016 |
| 3421668 | 0.64 | 0.001 | 1.3 | 0.58 | 0.0276 | 0.42 | 0.74 | 0.63 | 0.55 | 4.63 | 0.0331 | 0.0093 | 0.0031 | 0.04 | 0.017 |
| 3427811 | 0.61 | 0.0086 | 1.3 | 0.61 | 0.0018 | 0.54 | 0.68 | 0.64 | 0.59 | 3.76 | 0.0018 | 0.0186 | 0.0167 | 0.04 | 0.01 |
| 3427812 | 0.65 | 0.0002 | 1.51 | 0.6 | 0.0042 | 0.6 | 0.6 | 0.61 | 0.6 | 5.2 | 0.0055 | 0.0004 | 0.0003 | 0 | 0 |
| 3428711 | 0.66 | 0.0002 | 1.34 | 0.62 | 0.0011 | 0.46 | 0.79 | 0.69 | 0.58 | 4.49 | 0.0002 | 0.0011 | 0.0009 | 0.01 | 0.004 |
| 3428712 | 0.62 | 0.0025 | 1.31 | 0.58 | 0.0276 | 0.45 | 0.71 | 0.62 | 0.56 | 4.48 | 0.0242 | 0.0169 | 0.0072 | 0.18 | 0.028 |
| 3429794 | 0.62 | 0.003 | 1.26 | 0.62 | 0.0007 | 0.59 | 0.65 | 0.64 | 0.61 | 5.44 | 0.0006 | 0.0053 | 0.006 | 0.1 | 0.023 |
| 3430575 | 0.62 | 0.0025 | 1.26 | 0.61 | 0.0018 | 0.54 | 0.68 | 0.64 | 0.59 | 6.53 | 0.0021 | 0.0033 | 0.0034 | 0.07 | 0.02 |
| 3431352 | 0.6 | 0.0111 | 1.31 | 0.6 | 0.0042 | 0.58 | 0.62 | 0.61 | 0.59 | 5.54 | 0.0047 | 0.0188 | 0.0099 | 0.13 | 0.011 |
| 3431686 | 0.64 | 0.0007 | 1.27 | 0.64 | 0.0001 | 0.55 | 0.72 | 0.67 | 0.61 | 5.54 | 0.0002 | 0.0014 | 0.0008 | 0.05 | 0.004 |
| 3432171 | 0.64 | 0.0006 | 1.42 | 0.64 | 0.0001 | 0.63 | 0.64 | 0.65 | 0.63 | 5.86 | 0.0002 | 0.0005 | 0.0003 | 0.04 | 0.002 |
| 3432352 | 0.62 | 0.0049 | 1.26 | 0.58 | 0.0197 | 0.52 | 0.64 | 0.6 | 0.57 | 5.27 | 0.0135 | 0.0039 | 0.0068 | 0.06 | 0.018 |
| 3435860 | 0.64 | 0.0008 | 1.4 | 0.64 | 0.0001 | 0.67 | 0.6 | 0.64 | 0.64 | 4.87 | 0.0001 | 0.0008 | 0.001 | 0.03 | 0.006 |
| 3435864 | 0.64 | 0.0006 | 1.28 | 0.59 | 0.0138 | 0.5 | 0.67 | 0.61 | 0.57 | 6.2 | 0.0067 | 0.0005 | 0.0009 | 0.01 | 0.009 |
| 3436017 | 0.62 | 0.0025 | 1.25 | 0.59 | 0.0138 | 0.56 | 0.61 | 0.6 | 0.58 | 5.68 | 0.0145 | 0.004 | 0.0035 | 0.02 | 0.008 |
| 3436264 | 0.62 | 0.0031 | 1.48 | 0.6 | 0.0064 | 0.56 | 0.63 | 0.61 | 0.58 | 4.23 | 0.0064 | 0.0047 | 0.0035 | 0.03 | 0.003 |
| 3438044 | 0.61 | 0.0098 | 1.38 | 0.59 | 0.0095 | 0.48 | 0.71 | 0.63 | 0.57 | 4.94 | 0.0063 | 0.0105 | 0.0116 | 0.2 | 0.105 |
| 3445545 | 0.61 | 0.0065 | 1.27 | 0.59 | 0.0095 | 0.54 | 0.64 | 0.61 | 0.58 | 5.51 | 0.0066 | 0.0105 | 0.0078 | 0.12 | 0.02 |
| 3445674 | 0.64 | 0.0008 | 1.36 | 0.6 | 0.0064 | 0.47 | 0.73 | 0.64 | 0.57 | 3.86 | 0.0016 | 0.0034 | 0.0036 | 0.01 | 0.007 |
| 3445750 | 0.64 | 0.0009 | 1.3 | 0.6 | 0.0064 | 0.43 | 0.78 | 0.66 | 0.57 | 4.94 | 0.003 | 0.001 | 0.0008 | 0.47 | 0.142 |
| 3446488 | 0.61 | 0.0055 | 1.34 | 0.58 | 0.0197 | 0.58 | 0.58 | 0.59 | 0.58 | 5.85 | 0.0292 | 0.0049 | 0.0051 | 0.34 | 0.056 |
| 3446869 | 0.61 | 0.0097 | 1.25 | 0.6 | 0.0064 | 0.61 | 0.58 | 0.6 | 0.59 | 7.86 | 0.0106 | 0.0086 | 0.007 | 0.05 | 0.006 |
| 3446885 | 0.61 | 0.006 | 1.26 | 0.57 | 0.0513 | 0.48 | 0.66 | 0.59 | 0.55 | 4.75 | 0.0338 | 0.0069 | 0.0077 | 0.02 | 0.02 |
| 3452256 | 0.63 | 0.0022 | 1.34 | 0.62 | 0.0007 | 0.65 | 0.59 | 0.62 | 0.62 | 5.46 | 0.0014 | 0.0028 | 0.0021 | 0.05 | 0.017 |
| 3452262 | 0.63 | 0.0011 | 1.41 | 0.61 | 0.0028 | 0.56 | 0.65 | 0.63 | 0.59 | 4.74 | 0.0035 | 0.0018 | 0.0018 | 0.08 | 0.034 |
| 3452324 | 0.62 | 0.0036 | 1.31 | 0.61 | 0.0018 | 0.56 | 0.66 | 0.63 | 0.6 | 5.71 | 0.001 | 0.0029 | 0.0046 | 0.15 | 0.046 |
| 3452349 | 0.67 | 0 | 1.25 | 0.62 | 0.0007 | 0.51 | 0.73 | 0.67 | 0.6 | 6.28 | 0.0004 | 0.0001 | 0.0001 | 0.12 | 0.011 |
| 3454729 | 0.62 | 0.0024 | 1.26 | 0.61 | 0.0018 | 0.58 | 0.64 | 0.63 | 0.6 | 7.12 | 0.0011 | 0.0022 | 0.0025 | 0.08 | 0.01 |
| 3456599 | 0.63 | 0.0015 | 1.4 | 0.62 | 0.0011 | 0.59 | 0.64 | 0.63 | 0.61 | 4.31 | 0.0007 | 0.0019 | 0.0024 | 0.04 | 0.015 |
| 3457550 | 0.64 | 0.0008 | 1.27 | 0.64 | 0.0001 | 0.57 | 0.7 | 0.67 | 0.62 | 6.92 | 0.0002 | 0.0008 | 0.0006 | 0.05 | 0.008 |
| 3458494 | 0.63 | 0.0011 | 1.28 | 0.62 | 0.0011 | 0.56 | 0.67 | 0.64 | 0.6 | 5.36 | 0.0004 | 0.0003 | 0.0007 | 0 | 0.002 |
| 3461302 | 0.63 | 0.0021 | 1.4 | 0.6 | 0.0042 | 0.59 | 0.61 | 0.61 | 0.59 | 6.08 | 0.0057 | 0.0033 | 0.0023 | 0.05 | 0.003 |
| 3461431 | 0.68 | 0 | 1.28 | 0.66 | 0 | 0.6 | 0.71 | 0.69 | 0.64 | 4.02 | 0 | 0 | 0 | 0 | 0.001 |
| 3462710 | 0.61 | 0.0055 | 1.28 | 0.59 | 0.0095 | 0.54 | 0.64 | 0.61 | 0.58 | 6.36 | 0.0099 | 0.01 | 0.0079 | 0.1 | 0.025 |
| 3462846 | 0.62 | 0.0037 | 1.34 | 0.57 | 0.0513 | 0.52 | 0.61 | 0.58 | 0.56 | 4.1 | 0.0946 | 0.0243 | 0.0079 | 0.21 | 0.016 |
| 3462861 | 0.6 | 0.0107 | 1.26 | 0.56 | 0.0681 | 0.51 | 0.61 | 0.58 | 0.55 | 5.04 | 0.0861 | 0.017 | 0.0147 | 0.14 | 0.022 |
| 3462867 | 0.65 | 0.0003 | 1.31 | 0.62 | 0.0007 | 0.68 | 0.56 | 0.62 | 0.63 | 7.41 | 0.0009 | 0.0006 | 0.0005 | 0.02 | 0.001 |
| 3462868 | 0.63 | 0.0011 | 1.26 | 0.58 | 0.0197 | 0.46 | 0.71 | 0.62 | 0.56 | 4.64 | 0.0393 | 0.007 | 0.0029 | 0.21 | 0.024 |
| 3462884 | 0.65 | 0.0002 | 1.3 | 0.62 | 0.0007 | 0.5 | 0.74 | 0.67 | 0.59 | 5.57 | 0.0004 | 0.0008 | 0.0004 | 0.05 | 0.002 |
| 3462988 | 0.64 | 0.0005 | 1.34 | 0.62 | 0.0007 | 0.69 | 0.55 | 0.61 | 0.64 | 5.34 | 0.0008 | 0.0007 | 0.0005 | 0.01 | 0.003 |
| 3463596 | 0.68 | 0 | 1.29 | 0.64 | 0.0001 | 0.52 | 0.76 | 0.69 | 0.61 | 6.46 | 0.0001 | 0 | 0 | 0.01 | 0 |
| 3463603 | 0.67 | 0 | 1.27 | 0.63 | 0.0002 | 0.56 | 0.7 | 0.66 | 0.61 | 4.05 | 0.0001 | 0.0003 | 0.0006 | 0.01 | 0.002 |
| 3464009 | 0.61 | 0.0058 | 1.27 | 0.63 | 0.0004 | 0.66 | 0.59 | 0.63 | 0.63 | 4.22 | 0.001 | 0.0295 | 0.0147 | 0.47 | 0.096 |
| 3465251 | 0.63 | 0.0021 | 1.36 | 0.6 | 0.0042 | 0.5 | 0.7 | 0.64 | 0.58 | 5.2 | 0.0027 | 0.0046 | 0.0045 | 0.12 | 0.023 |
| 3465252 | 0.61 | 0.007 | 1.47 | 0.62 | 0.0011 | 0.61 | 0.62 | 0.63 | 0.61 | 6.33 | 0.0022 | 0.0053 | 0.0044 | 0.15 | 0.018 |
| 3465278 | 0.63 | 0.002 | 1.29 | 0.53 | 0.31 | 0.31 | 0.76 | 0.56 | 0.51 | 5.11 | 0.3312 | 0.0161 | 0.0179 | 0.3 | 0.166 |
| 3466861 | 0.62 | 0.0027 | 1.27 | 0.6 | 0.0042 | 0.46 | 0.76 | 0.66 | 0.57 | 4.55 | 0.0009 | 0.0015 | 0.0034 | 0.13 | 0.04 |
| 3467642 | 0.63 | 0.002 | 1.36 | 0.61 | 0.0028 | 0.59 | 0.62 | 0.62 | 0.6 | 5.79 | 0.0024 | 0.0056 | 0.0029 | 0.1 | 0.015 |
| 3468015 | 0.63 | 0.0018 | 1.39 | 0.62 | 0.0011 | 0.52 | 0.71 | 0.65 | 0.59 | 4.88 | 0.0003 | 0.0019 | 0.0029 | 0.04 | 0.009 |
| 3469369 | 0.65 | 0.0004 | 1.27 | 0.6 | 0.0042 | 0.48 | 0.73 | 0.65 | 0.58 | 3.12 | 0.0033 | 0.0013 | 0.0016 | 0.01 | 0.012 |
| 3471177 | 0.6 | 0.0141 | 1.26 | 0.59 | 0.0138 | 0.5 | 0.67 | 0.61 | 0.57 | 4.52 | 0.008 | 0.0075 | 0.0107 | 0.23 | 0.184 |
| 3471377 | 0.64 | 0.0008 | 1.45 | 0.64 | 0.0001 | 0.6 | 0.68 | 0.66 | 0.63 | 6.02 | 0.0001 | 0.0003 | 0.0004 | 0.05 | 0.003 |
| 3471379 | 0.62 | 0.0035 | 1.27 | 0.58 | 0.0197 | 0.49 | 0.68 | 0.61 | 0.56 | 4.35 | 0.0131 | 0.0067 | 0.0081 | 0.51 | 0.146 |
| 3472092 | 0.62 | 0.0043 | 1.29 | 0.61 | 0.0018 | 0.65 | 0.57 | 0.61 | 0.62 | 7.95 | 0.0025 | 0.0042 | 0.0034 | 0.12 | 0.009 |
| 3473482 | 0.62 | 0.0043 | 1.3 | 0.57 | 0.038 | 0.46 | 0.69 | 0.61 | 0.55 | 4.38 | 0.0518 | 0.0078 | 0.0068 | 0.11 | 0.02 |
| 3474494 | 0.65 | 0.0004 | 1.33 | 0.62 | 0.0007 | 0.65 | 0.59 | 0.62 | 0.62 | 6.29 | 0.0019 | 0.0008 | 0.0005 | 0.04 | 0.004 |
| 3474937 | 0.61 | 0.0079 | 1.26 | 0.58 | 0.0276 | 0.52 | 0.63 | 0.6 | 0.56 | 5.34 | 0.0197 | 0.0188 | 0.0152 | 0.06 | 0.009 |
| 3475623 | 0.61 | 0.0058 | 1.26 | 0.57 | 0.038 | 0.46 | 0.69 | 0.61 | 0.55 | 4.6 | 0.0285 | 0.0095 | 0.0102 | 0.1 | 0.017 |
| 3475737 | 0.62 | 0.004 | 1.25 | 0.64 | 0.0001 | 0.55 | 0.72 | 0.67 | 0.61 | 4.86 | 0.0001 | 0.0043 | 0.0066 | 0.07 | 0.046 |
| 3476294 | 0.62 | 0.0029 | 1.38 | 0.61 | 0.0028 | 0.61 | 0.6 | 0.61 | 0.6 | 5.71 | 0.0039 | 0.0046 | 0.0034 | 0.06 | 0.004 |
| 3480098 | 0.66 | 0.0002 | 1.45 | 0.6 | 0.0064 | 0.55 | 0.64 | 0.62 | 0.58 | 4.26 | 0.0031 | 0.0001 | 0.0002 | 0.06 | 0.007 |
| 3480100 | 0.63 | 0.0016 | 1.25 | 0.58 | 0.0276 | 0.48 | 0.68 | 0.61 | 0.56 | 3.35 | 0.016 | 0.0014 | 0.002 | 0.02 | 0.01 |
| 3480173 | 0.63 | 0.0014 | 1.25 | 0.6 | 0.0042 | 0.51 | 0.69 | 0.63 | 0.58 | 5.38 | 0.002 | 0.0014 | 0.0018 | 0.07 | 0.01 |
| 3480201 | 0.65 | 0.0003 | 1.37 | 0.62 | 0.0007 | 0.62 | 0.62 | 0.63 | 0.62 | 5.14 | 0.0006 | 0.0003 | 0.0004 | 0.09 | 0.009 |
| 3480203 | 0.62 | 0.003 | 1.27 | 0.59 | 0.0138 | 0.59 | 0.58 | 0.59 | 0.58 | 5.88 | 0.0211 | 0.007 | 0.004 | 0.18 | 0.008 |
| 3480685 | 0.63 | 0.002 | 1.3 | 0.63 | 0.0002 | 0.66 | 0.6 | 0.63 | 0.63 | 7 | 0.0002 | 0.0025 | 0.0022 | 0.03 | 0.005 |
| 3480856 | 0.67 | 0.0001 | 1.29 | 0.61 | 0.0018 | 0.58 | 0.64 | 0.63 | 0.6 | 6.68 | 0.0039 | 0.0002 | 0.0001 | 0.02 | 0.003 |
| 3481480 | 0.64 | 0.0008 | 1.35 | 0.61 | 0.0018 | 0.59 | 0.63 | 0.63 | 0.6 | 4.71 | 0.0028 | 0.0042 | 0.0017 | 0.05 | 0.001 |
| 3482124 | 0.61 | 0.0063 | 1.28 | 0.59 | 0.0138 | 0.51 | 0.66 | 0.61 | 0.57 | 5.98 | 0.0166 | 0.0035 | 0.0051 | 0.36 | 0.126 |
| 3482915 | 0.62 | 0.0047 | 1.43 | 0.62 | 0.0007 | 0.53 | 0.71 | 0.66 | 0.6 | 4.94 | 0.0002 | 0.007 | 0.0076 | 0.03 | 0.004 |
| 3482916 | 0.61 | 0.0061 | 1.56 | 0.63 | 0.0004 | 0.63 | 0.62 | 0.63 | 0.62 | 4.71 | 0.0004 | 0.0041 | 0.0031 | 0.1 | 0.014 |
| 3483215 | 0.64 | 0.0006 | 1.27 | 0.62 | 0.0007 | 0.62 | 0.62 | 0.63 | 0.62 | 7.97 | 0.0005 | 0.0006 | 0.0008 | 0.03 | 0.003 |
| 3484743 | 0.65 | 0.0004 | 1.32 | 0.66 | 0 | 0.63 | 0.68 | 0.67 | 0.64 | 6.65 | 0 | 0.0004 | 0.0003 | 0.02 | 0.001 |
| 3484750 | 0.65 | 0.0002 | 1.48 | 0.64 | 0.0001 | 0.6 | 0.68 | 0.66 | 0.63 | 5.16 | 0.0001 | 0.0004 | 0.0003 | 0.01 | 0.002 |
| 3484756 | 0.64 | 0.0009 | 1.27 | 0.62 | 0.0011 | 0.55 | 0.68 | 0.64 | 0.6 | 5.07 | 0.0005 | 0.0007 | 0.0008 | 0.1 | 0.018 |
| 3484762 | 0.66 | 0.0001 | 1.33 | 0.64 | 0.0001 | 0.58 | 0.7 | 0.67 | 0.62 | 6.04 | 0 | 0.0001 | 0.0001 | 0.01 | 0.001 |
| 3485891 | 0.61 | 0.0092 | 1.54 | 0.59 | 0.0095 | 0.56 | 0.62 | 0.61 | 0.58 | 4.59 | 0.0142 | 0.0118 | 0.0077 | 0.16 | 0.012 |
| 3489010 | 0.62 | 0.0034 | 1.38 | 0.58 | 0.0276 | 0.56 | 0.59 | 0.59 | 0.57 | 5.21 | 0.0421 | 0.0074 | 0.0052 | 0.07 | 0.019 |
| 3489011 | 0.62 | 0.0026 | 1.33 | 0.61 | 0.0018 | 0.7 | 0.52 | 0.6 | 0.63 | 7.21 | 0.0034 | 0.0043 | 0.0025 | 0.11 | 0.007 |
| 3494193 | 0.61 | 0.0096 | 1.39 | 0.61 | 0.0018 | 0.62 | 0.6 | 0.62 | 0.61 | 5.2 | 0.001 | 0.0067 | 0.01 | 0.06 | 0.028 |
| 3497656 | 0.6 | 0.0148 | 1.36 | 0.6 | 0.0064 | 0.53 | 0.66 | 0.62 | 0.58 | 3.74 | 0.0131 | 0.0094 | 0.0103 | 0.12 | 0.051 |
| 3497658 | 0.64 | 0.0009 | 1.27 | 0.64 | 0.0001 | 0.59 | 0.68 | 0.66 | 0.62 | 6.46 | 0.0001 | 0.001 | 0.001 | 0.02 | 0.001 |
| 3497823 | 0.65 | 0.0003 | 1.31 | 0.61 | 0.0018 | 0.55 | 0.67 | 0.64 | 0.59 | 4.76 | 0.0017 | 0.0009 | 0.0007 | 0.02 | 0.003 |
| 3498048 | 0.63 | 0.0014 | 1.36 | 0.59 | 0.0138 | 0.45 | 0.73 | 0.63 | 0.56 | 4.52 | 0.0084 | 0.0006 | 0.0008 | 0.04 | 0.002 |
| 3498445 | 0.64 | 0.0007 | 1.31 | 0.63 | 0.0004 | 0.62 | 0.63 | 0.64 | 0.62 | 5.36 | 0.0006 | 0.0006 | 0.0008 | 0.05 | 0.007 |
| 3498523 | 0.66 | 0.0001 | 1.67 | 0.65 | 0 | 0.65 | 0.64 | 0.65 | 0.64 | 5.42 | 0.0001 | 0.0004 | 0.0002 | 0.02 | 0.002 |
| 3498537 | 0.62 | 0.0033 | 1.26 | 0.59 | 0.0138 | 0.51 | 0.66 | 0.61 | 0.57 | 5.14 | 0.0083 | 0.0041 | 0.0041 | 0.13 | 0.025 |
| 3498977 | 0.64 | 0.0008 | 1.34 | 0.59 | 0.0095 | 0.61 | 0.57 | 0.6 | 0.59 | 5.28 | 0.013 | 0.001 | 0.0006 | 0.02 | 0.001 |
| 3499545 | 0.62 | 0.0024 | 1.29 | 0.63 | 0.0002 | 0.62 | 0.64 | 0.64 | 0.62 | 5.41 | 0.0003 | 0.0029 | 0.0029 | 0.06 | 0.006 |
| 3500408 | 0.61 | 0.0076 | 1.3 | 0.59 | 0.0095 | 0.53 | 0.65 | 0.61 | 0.58 | 5.14 | 0.0086 | 0.0039 | 0.006 | 0.1 | 0.013 |
| 3501216 | 0.64 | 0.0009 | 1.29 | 0.59 | 0.0095 | 0.55 | 0.63 | 0.61 | 0.58 | 6.18 | 0.0181 | 0.001 | 0.0017 | 0.04 | 0.006 |
| 3501217 | 0.65 | 0.0002 | 1.34 | 0.61 | 0.0028 | 0.63 | 0.58 | 0.61 | 0.61 | 6.24 | 0.0049 | 0.0002 | 0.0003 | 0.01 | 0.002 |
| 3501555 | 0.68 | 0 | 1.51 | 0.63 | 0.0002 | 0.57 | 0.69 | 0.66 | 0.61 | 5.52 | 0.0002 | 0.0001 | 0 | 0.01 | 0 |
| 3503211 | 0.65 | 0.0002 | 1.27 | 0.62 | 0.0007 | 0.56 | 0.68 | 0.65 | 0.6 | 6.53 | 0.0006 | 0.0003 | 0.0003 | 0.02 | 0.003 |
| 3505453 | 0.61 | 0.0073 | 1.56 | 0.6 | 0.0042 | 0.55 | 0.65 | 0.62 | 0.59 | 4.72 | 0.0119 | 0.01 | 0.0045 | 0.12 | 0.008 |
| 3506941 | 0.62 | 0.0043 | 1.38 | 0.61 | 0.0028 | 0.56 | 0.65 | 0.63 | 0.59 | 4.83 | 0.0038 | 0.0043 | 0.0049 | 0.13 | 0.027 |
| 3508731 | 0.65 | 0.0002 | 1.35 | 0.58 | 0.0197 | 0.33 | 0.85 | 0.69 | 0.55 | 4.44 | 0.0019 | 0.0006 | 0.0009 | 0.03 | 0.006 |
| 3508732 | 0.61 | 0.0077 | 1.28 | 0.62 | 0.0011 | 0.54 | 0.69 | 0.65 | 0.6 | 5.69 | 0.0006 | 0.0062 | 0.0072 | 0.15 | 0.043 |
| 3508761 | 0.65 | 0.0002 | 1.31 | 0.63 | 0.0004 | 0.61 | 0.64 | 0.64 | 0.62 | 6.68 | 0.0003 | 0.0002 | 0.0004 | 0.02 | 0.006 |
| 3509912 | 0.64 | 0.0008 | 1.48 | 0.61 | 0.0018 | 0.61 | 0.61 | 0.62 | 0.61 | 3.92 | 0.0015 | 0.0034 | 0.0022 | 0.04 | 0.004 |
| 3509913 | 0.61 | 0.0085 | 1.32 | 0.6 | 0.0064 | 0.5 | 0.69 | 0.63 | 0.58 | 4.02 | 0.0082 | 0.0161 | 0.0134 | 0.16 | 0.035 |
| 3510070 | 0.61 | 0.0082 | 1.25 | 0.61 | 0.0018 | 0.5 | 0.72 | 0.65 | 0.59 | 7.05 | 0.0005 | 0.0024 | 0.0073 | 0.07 | 0.017 |
| 3510382 | 0.64 | 0.0005 | 1.37 | 0.61 | 0.0028 | 0.56 | 0.65 | 0.63 | 0.59 | 5.76 | 0.0032 | 0.0005 | 0.0007 | 0.01 | 0.002 |
| 3512558 | 0.64 | 0.001 | 1.28 | 0.59 | 0.0095 | 0.65 | 0.53 | 0.59 | 0.6 | 7.23 | 0.0117 | 0.001 | 0.001 | 0.07 | 0.005 |
| 3513710 | 0.62 | 0.0028 | 1.29 | 0.59 | 0.0138 | 0.5 | 0.68 | 0.62 | 0.57 | 4.27 | 0.0154 | 0.0068 | 0.0034 | 0.09 | 0.013 |
| 3517677 | 0.65 | 0.0002 | 1.45 | 0.61 | 0.0018 | 0.37 | 0.87 | 0.74 | 0.57 | 4.15 | 0.0001 | 0.0001 | 0.0002 | 0.03 | 0.003 |
| 3517683 | 0.67 | 0 | 1.25 | 0.64 | 0.0001 | 0.53 | 0.76 | 0.69 | 0.61 | 5.6 | 0 | 0.0001 | 0.0003 | 0.01 | 0.003 |
| 3518501 | 0.62 | 0.0038 | 1.35 | 0.59 | 0.0138 | 0.49 | 0.69 | 0.62 | 0.57 | 4.62 | 0.0038 | 0.013 | 0.0114 | 0.03 | 0.01 |
| 3519136 | 0.63 | 0.001 | 1.35 | 0.63 | 0.0004 | 0.53 | 0.72 | 0.67 | 0.6 | 4.81 | 0.0003 | 0.0022 | 0.0017 | 0.13 | 0.012 |
| 3521962 | 0.61 | 0.0059 | 1.27 | 0.61 | 0.0028 | 0.49 | 0.73 | 0.65 | 0.58 | 5.22 | 0.0007 | 0.003 | 0.0042 | 0.29 | 0.079 |
| 3524627 | 0.62 | 0.0044 | 1.26 | 0.6 | 0.0042 | 0.54 | 0.66 | 0.63 | 0.59 | 5.82 | 0.0032 | 0.0044 | 0.0055 | 0.07 | 0.013 |
| 3524630 | 0.6 | 0.0141 | 1.31 | 0.6 | 0.0064 | 0.53 | 0.66 | 0.62 | 0.58 | 4.61 | 0.0078 | 0.02 | 0.0214 | 0.45 | 0.127 |
| 3524631 | 0.64 | 0.0007 | 1.39 | 0.61 | 0.0018 | 0.58 | 0.64 | 0.63 | 0.6 | 4.94 | 0.0015 | 0.0007 | 0.0005 | 0.08 | 0.005 |
| 3525314 | 0.65 | 0.0003 | 1.37 | 0.62 | 0.0007 | 0.61 | 0.63 | 0.63 | 0.61 | 6.22 | 0.0007 | 0.0004 | 0.0004 | 0.02 | 0.001 |
| 3525317 | 0.65 | 0.0002 | 1.47 | 0.59 | 0.0095 | 0.55 | 0.63 | 0.61 | 0.58 | 5.43 | 0.0188 | 0.0002 | 0.0002 | 0.01 | 0 |
| 3525320 | 0.63 | 0.0016 | 1.38 | 0.6 | 0.0042 | 0.56 | 0.64 | 0.62 | 0.59 | 5.97 | 0.0074 | 0.0009 | 0.0012 | 0.1 | 0.019 |
| 3525547 | 0.61 | 0.0079 | 1.26 | 0.6 | 0.0042 | 0.67 | 0.53 | 0.6 | 0.61 | 5.66 | 0.0108 | 0.0083 | 0.0065 | 0.07 | 0.007 |
| 3525702 | 0.62 | 0.0024 | 1.37 | 0.62 | 0.0011 | 0.54 | 0.69 | 0.65 | 0.6 | 5.08 | 0.001 | 0.0037 | 0.002 | 0.11 | 0.011 |
| 3525704 | 0.65 | 0.0002 | 1.32 | 0.61 | 0.0018 | 0.67 | 0.55 | 0.61 | 0.62 | 4.82 | 0.0019 | 0.0004 | 0.0003 | 0.03 | 0.006 |
| 3525710 | 0.64 | 0.0005 | 1.29 | 0.64 | 0.0001 | 0.62 | 0.65 | 0.65 | 0.63 | 8.03 | 0.0002 | 0.0006 | 0.0004 | 0.03 | 0.001 |
| 3526383 | 0.63 | 0.0019 | 1.25 | 0.59 | 0.0095 | 0.46 | 0.73 | 0.64 | 0.57 | 3.04 | 0.0052 | 0.0499 | 0.0366 | 0.13 | 0.027 |
| 3527530 | 0.62 | 0.0034 | 1.33 | 0.6 | 0.0064 | 0.6 | 0.59 | 0.6 | 0.59 | 4.61 | 0.0063 | 0.0049 | 0.0036 | 0.01 | 0.002 |
| 3528208 | 0.6 | 0.0135 | 1.35 | 0.6 | 0.0064 | 0.56 | 0.63 | 0.61 | 0.58 | 4.77 | 0.0043 | 0.0318 | 0.0225 | 0.46 | 0.13 |
| 3529094 | 0.62 | 0.0039 | 1.28 | 0.62 | 0.0011 | 0.62 | 0.61 | 0.62 | 0.61 | 4.74 | 0.002 | 0.0044 | 0.0045 | 0.14 | 0.031 |
| 3531094 | 0.63 | 0.0013 | 1.33 | 0.57 | 0.038 | 0.5 | 0.64 | 0.59 | 0.56 | 5.34 | 0.0448 | 0.0041 | 0.0028 | 0.21 | 0.032 |
| 3531491 | 0.65 | 0.0003 | 1.47 | 0.63 | 0.0002 | 0.54 | 0.72 | 0.67 | 0.61 | 4.11 | 0.0001 | 0.0003 | 0.0003 | 0.1 | 0.007 |
| 3533452 | 0.64 | 0.0009 | 1.37 | 0.62 | 0.0007 | 0.64 | 0.6 | 0.63 | 0.62 | 6.32 | 0.001 | 0.0009 | 0.0009 | 0.03 | 0.002 |
| 3534950 | 0.68 | 0 | 1.41 | 0.65 | 0 | 0.61 | 0.68 | 0.67 | 0.63 | 6.12 | 0 | 0 | 0 | 0.01 | 0.001 |
| 3535917 | 0.61 | 0.0084 | 1.25 | 0.59 | 0.0138 | 0.5 | 0.67 | 0.61 | 0.57 | 4.79 | 0.0108 | 0.0196 | 0.0119 | 0.09 | 0.016 |
| 3536669 | 0.64 | 0.0006 | 1.26 | 0.65 | 0 | 0.52 | 0.79 | 0.72 | 0.62 | 6.07 | 0 | 0.0005 | 0.0005 | 0.01 | 0.001 |
| 3536745 | 0.64 | 0.0004 | 1.41 | 0.59 | 0.0095 | 0.5 | 0.68 | 0.62 | 0.57 | 4.96 | 0.0209 | 0.0025 | 0.0011 | 0.04 | 0.008 |
| 3536931 | 0.63 | 0.0021 | 1.29 | 0.59 | 0.0095 | 0.56 | 0.62 | 0.61 | 0.58 | 7.39 | 0.009 | 0.0024 | 0.0027 | 0.12 | 0.021 |
| 3536934 | 0.61 | 0.0084 | 1.29 | 0.62 | 0.0007 | 0.6 | 0.64 | 0.64 | 0.61 | 4.7 | 0.0008 | 0.0072 | 0.0078 | 0.08 | 0.019 |
| 3536938 | 0.61 | 0.0072 | 1.32 | 0.6 | 0.0042 | 0.57 | 0.63 | 0.62 | 0.59 | 5.98 | 0.007 | 0.0051 | 0.0062 | 0.12 | 0.039 |
| 3536943 | 0.66 | 0.0001 | 1.31 | 0.63 | 0.0002 | 0.6 | 0.66 | 0.65 | 0.62 | 5.91 | 0.0001 | 0 | 0.0001 | 0.01 | 0.001 |
| 3536948 | 0.64 | 0.0006 | 1.26 | 0.6 | 0.0064 | 0.53 | 0.66 | 0.62 | 0.58 | 6.2 | 0.0065 | 0.0005 | 0.0009 | 0.04 | 0.013 |
| 3536949 | 0.61 | 0.0058 | 1.52 | 0.6 | 0.0042 | 0.6 | 0.6 | 0.61 | 0.6 | 5.83 | 0.0056 | 0.003 | 0.0036 | 0.03 | 0.007 |
| 3536951 | 0.62 | 0.0038 | 1.49 | 0.59 | 0.0095 | 0.55 | 0.63 | 0.61 | 0.58 | 4.41 | 0.011 | 0.004 | 0.0045 | 0.04 | 0.014 |
| 3536992 | 0.61 | 0.0055 | 1.32 | 0.58 | 0.0276 | 0.48 | 0.68 | 0.61 | 0.56 | 3.98 | 0.0599 | 0.006 | 0.004 | 0.21 | 0.026 |
| 3536994 | 0.64 | 0.0007 | 1.32 | 0.62 | 0.0007 | 0.57 | 0.67 | 0.64 | 0.61 | 6.35 | 0.0012 | 0.001 | 0.0007 | 0.05 | 0.003 |
| 3537787 | 0.65 | 0.0004 | 1.29 | 0.65 | 0 | 0.55 | 0.74 | 0.69 | 0.62 | 5.2 | 0 | 0.0004 | 0.0003 | 0.1 | 0.008 |
| 3537795 | 0.6 | 0.0107 | 1.61 | 0.61 | 0.0028 | 0.63 | 0.58 | 0.61 | 0.61 | 5.36 | 0.0027 | 0.0086 | 0.0055 | 0.06 | 0.008 |
| 3537796 | 0.62 | 0.0047 | 1.5 | 0.59 | 0.0095 | 0.63 | 0.55 | 0.59 | 0.59 | 4.67 | 0.0113 | 0.0098 | 0.0057 | 0.03 | 0.004 |
| 3539104 | 0.63 | 0.0019 | 1.25 | 0.58 | 0.0197 | 0.52 | 0.64 | 0.6 | 0.57 | 6.42 | 0.0328 | 0.002 | 0.002 | 0.04 | 0.005 |
| 3539877 | 0.63 | 0.0014 | 1.34 | 0.6 | 0.0064 | 0.52 | 0.67 | 0.62 | 0.58 | 4.21 | 0.0136 | 0.0089 | 0.0043 | 0.07 | 0.018 |
| 3543929 | 0.64 | 0.0004 | 1.31 | 0.6 | 0.0064 | 0.66 | 0.53 | 0.59 | 0.6 | 6.37 | 0.0111 | 0.0007 | 0.0003 | 0.03 | 0.002 |
| 3544473 | 0.62 | 0.0024 | 1.3 | 0.61 | 0.0018 | 0.57 | 0.65 | 0.63 | 0.6 | 6.47 | 0.0017 | 0.0036 | 0.0026 | 0.03 | 0.003 |
| 3544890 | 0.61 | 0.0079 | 1.41 | 0.58 | 0.0197 | 0.53 | 0.63 | 0.6 | 0.57 | 4.49 | 0.0381 | 0.0078 | 0.0065 | 0.55 | 0.132 |
| 3545534 | 0.61 | 0.0052 | 1.37 | 0.61 | 0.0018 | 0.62 | 0.6 | 0.62 | 0.61 | 6.34 | 0.0022 | 0.0048 | 0.0038 | 0.04 | 0.005 |
| 3549043 | 0.61 | 0.0101 | 1.29 | 0.58 | 0.0276 | 0.51 | 0.64 | 0.6 | 0.56 | 5.29 | 0.0412 | 0.0149 | 0.0115 | 0.34 | 0.072 |
| 3550451 | 0.62 | 0.0044 | 1.37 | 0.63 | 0.0004 | 0.62 | 0.63 | 0.64 | 0.62 | 5.63 | 0.0003 | 0.0025 | 0.004 | 0.05 | 0.009 |
| 3550453 | 0.64 | 0.0009 | 1.26 | 0.63 | 0.0002 | 0.58 | 0.68 | 0.66 | 0.61 | 5.12 | 0.0002 | 0.0008 | 0.0013 | 0.06 | 0.038 |
| 3550455 | 0.61 | 0.0055 | 1.25 | 0.57 | 0.0513 | 0.42 | 0.72 | 0.61 | 0.55 | 3.91 | 0.0535 | 0.0147 | 0.0106 | 0.41 | 0.12 |
| 3552937 | 0.61 | 0.01 | 1.3 | 0.58 | 0.0197 | 0.56 | 0.6 | 0.59 | 0.57 | 4.66 | 0.0167 | 0.008 | 0.0155 | 0.22 | 0.153 |
| 3552938 | 0.61 | 0.0065 | 1.31 | 0.57 | 0.038 | 0.51 | 0.63 | 0.59 | 0.56 | 5.16 | 0.0371 | 0.0054 | 0.0083 | 0.04 | 0.02 |
| 3556198 | 0.64 | 0.0004 | 1.26 | 0.61 | 0.0018 | 0.56 | 0.66 | 0.63 | 0.6 | 7.24 | 0.001 | 0.0007 | 0.0005 | 0.05 | 0.007 |
| 3556224 | 0.65 | 0.0003 | 1.28 | 0.62 | 0.0007 | 0.62 | 0.62 | 0.63 | 0.62 | 5.68 | 0.0011 | 0.0009 | 0.0005 | 0.06 | 0.002 |
| 3556278 | 0.63 | 0.0019 | 1.34 | 0.61 | 0.0018 | 0.45 | 0.79 | 0.68 | 0.58 | 4.31 | 0.0008 | 0.002 | 0.0015 | 0.05 | 0.008 |
| 3556420 | 0.61 | 0.0066 | 1.35 | 0.61 | 0.0028 | 0.57 | 0.64 | 0.62 | 0.59 | 4.26 | 0.003 | 0.0071 | 0.0058 | 0.19 | 0.024 |
| 3558292 | 0.64 | 0.0008 | 1.32 | 0.62 | 0.0007 | 0.69 | 0.55 | 0.61 | 0.64 | 6.56 | 0.0009 | 0.0015 | 0.0011 | 0.03 | 0.003 |
| 3559519 | 0.64 | 0.0007 | 1.31 | 0.59 | 0.0138 | 0.42 | 0.77 | 0.65 | 0.56 | 4.33 | 0.0104 | 0.0009 | 0.0007 | 0.18 | 0.005 |
| 3559556 | 0.6 | 0.0117 | 1.25 | 0.58 | 0.0276 | 0.48 | 0.68 | 0.61 | 0.56 | 4.19 | 0.0242 | 0.0098 | 0.014 | 0.12 | 0.011 |
| 3562436 | 0.7 | 0 | 1.31 | 0.64 | 0.0001 | 0.49 | 0.81 | 0.72 | 0.6 | 3.49 | 0 | 0 | 0.0001 | 0.05 | 0.002 |
| 3563349 | 0.65 | 0.0004 | 1.26 | 0.63 | 0.0004 | 0.53 | 0.72 | 0.67 | 0.6 | 4.98 | 0.0001 | 0.0005 | 0.0007 | 0.04 | 0.009 |
| 3563970 | 0.62 | 0.0038 | 1.27 | 0.57 | 0.0513 | 0.46 | 0.68 | 0.6 | 0.55 | 4.02 | 0.0384 | 0.0049 | 0.0049 | 0.13 | 0.029 |
| 3566596 | 0.63 | 0.0019 | 1.39 | 0.62 | 0.0007 | 0.63 | 0.61 | 0.63 | 0.62 | 6.08 | 0.0005 | 0.0017 | 0.0015 | 0.02 | 0.002 |
| 3566603 | 0.6 | 0.0136 | 1.34 | 0.59 | 0.0138 | 0.64 | 0.53 | 0.59 | 0.59 | 5.5 | 0.0183 | 0.0177 | 0.0122 | 0.09 | 0.012 |
| 3566648 | 0.61 | 0.0076 | 1.26 | 0.58 | 0.0276 | 0.46 | 0.7 | 0.61 | 0.56 | 4.37 | 0.028 | 0.0061 | 0.0076 | 0.12 | 0.015 |
| 3566657 | 0.62 | 0.0033 | 1.32 | 0.6 | 0.0064 | 0.48 | 0.72 | 0.64 | 0.57 | 4.58 | 0.0013 | 0.0018 | 0.0042 | 0.01 | 0.012 |
| 3566993 | 0.61 | 0.007 | 1.29 | 0.58 | 0.0276 | 0.55 | 0.6 | 0.59 | 0.57 | 5.09 | 0.0416 | 0.0098 | 0.0087 | 0.08 | 0.022 |
| 3569346 | 0.65 | 0.0003 | 1.39 | 0.62 | 0.0007 | 0.52 | 0.72 | 0.66 | 0.6 | 4.98 | 0.0007 | 0.0003 | 0.0004 | 0.04 | 0.007 |
| 3569757 | 0.65 | 0.0002 | 1.32 | 0.61 | 0.0018 | 0.54 | 0.68 | 0.64 | 0.59 | 6.32 | 0.0019 | 0.0006 | 0.0003 | 0.02 | 0.001 |
| 3569759 | 0.65 | 0.0003 | 1.25 | 0.61 | 0.0018 | 0.57 | 0.65 | 0.63 | 0.6 | 5.88 | 0.0018 | 0.0003 | 0.0004 | 0.01 | 0.004 |
| 3569819 | 0.64 | 0.0006 | 1.31 | 0.61 | 0.0018 | 0.56 | 0.66 | 0.63 | 0.6 | 5.21 | 0.003 | 0.0022 | 0.0017 | 0.18 | 0.027 |
| 3570456 | 0.61 | 0.0066 | 1.25 | 0.61 | 0.0028 | 0.64 | 0.57 | 0.61 | 0.61 | 5.71 | 0.0039 | 0.0099 | 0.0077 | 0.06 | 0.009 |
| 3573155 | 0.6 | 0.0135 | 1.26 | 0.57 | 0.0513 | 0.53 | 0.6 | 0.58 | 0.56 | 4.88 | 0.058 | 0.0281 | 0.0173 | 0.22 | 0.031 |
| 3576551 | 0.61 | 0.0053 | 1.29 | 0.59 | 0.0095 | 0.65 | 0.53 | 0.59 | 0.6 | 5.7 | 0.0219 | 0.0069 | 0.0042 | 0.08 | 0.009 |
| 3576567 | 0.65 | 0.0003 | 1.43 | 0.62 | 0.0007 | 0.51 | 0.73 | 0.67 | 0.6 | 4.66 | 0.0001 | 0.0003 | 0.0006 | 0.01 | 0.004 |
| 3576712 | 0.62 | 0.0027 | 1.44 | 0.6 | 0.0064 | 0.51 | 0.68 | 0.63 | 0.58 | 4.77 | 0.0039 | 0.0016 | 0.0019 | 0.09 | 0.046 |
| 3576730 | 0.62 | 0.0031 | 1.38 | 0.6 | 0.0064 | 0.56 | 0.63 | 0.61 | 0.58 | 5.46 | 0.0092 | 0.0027 | 0.0025 | 0.11 | 0.066 |
| 3576939 | 0.63 | 0.0022 | 1.4 | 0.61 | 0.0028 | 0.68 | 0.53 | 0.6 | 0.62 | 5.89 | 0.003 | 0.0023 | 0.001 | 0.06 | 0.002 |
| 3576940 | 0.64 | 0.001 | 1.32 | 0.61 | 0.0028 | 0.56 | 0.65 | 0.63 | 0.59 | 5.96 | 0.0033 | 0.0018 | 0.0012 | 0.07 | 0.008 |
| 3577874 | 0.66 | 0.0001 | 1.42 | 0.62 | 0.0011 | 0.59 | 0.64 | 0.63 | 0.61 | 4.39 | 0.0022 | 0.0004 | 0.0002 | 0 | 0 |
| 3577898 | 0.63 | 0.0022 | 1.33 | 0.63 | 0.0002 | 0.63 | 0.63 | 0.64 | 0.63 | 5.37 | 0.0002 | 0.0017 | 0.0015 | 0.04 | 0.007 |
| 3577911 | 0.65 | 0.0002 | 1.29 | 0.64 | 0.0001 | 0.56 | 0.72 | 0.68 | 0.62 | 5.09 | 0 | 0.0002 | 0.0006 | 0.02 | 0.006 |
| 3578431 | 0.64 | 0.0005 | 1.3 | 0.63 | 0.0004 | 0.55 | 0.7 | 0.66 | 0.61 | 4.99 | 0.0004 | 0.0008 | 0.0007 | 0.06 | 0.004 |
| 3580188 | 0.61 | 0.0066 | 1.29 | 0.58 | 0.0276 | 0.52 | 0.63 | 0.6 | 0.56 | 5.28 | 0.027 | 0.0034 | 0.0083 | 0.04 | 0.038 |
| 3580204 | 0.61 | 0.0096 | 1.27 | 0.61 | 0.0018 | 0.55 | 0.67 | 0.64 | 0.59 | 6.72 | 0.0008 | 0.0056 | 0.006 | 0.05 | 0.013 |
| 3580587 | 0.62 | 0.0024 | 1.32 | 0.59 | 0.0095 | 0.52 | 0.66 | 0.62 | 0.58 | 3.5 | 0.0054 | 0.0029 | 0.0049 | 0.29 | 0.226 |
| 3580953 | 0.63 | 0.0015 | 1.27 | 0.58 | 0.0197 | 0.71 | 0.45 | 0.57 | 0.6 | 7.14 | 0.0262 | 0.006 | 0.005 | 0.09 | 0.008 |
| 3589660 | 0.61 | 0.0093 | 1.29 | 0.58 | 0.0276 | 0.46 | 0.7 | 0.61 | 0.56 | 4.64 | 0.0305 | 0.0167 | 0.0135 | 0.52 | 0.111 |
| 3590159 | 0.62 | 0.0042 | 1.29 | 0.6 | 0.0064 | 0.62 | 0.57 | 0.6 | 0.6 | 5.62 | 0.0063 | 0.0034 | 0.004 | 0.01 | 0.002 |
| 3590352 | 0.62 | 0.003 | 1.27 | 0.57 | 0.0513 | 0.51 | 0.62 | 0.58 | 0.55 | 5.27 | 0.0812 | 0.0043 | 0.0045 | 0.21 | 0.11 |
| 3591851 | 0.63 | 0.0014 | 1.28 | 0.6 | 0.0042 | 0.59 | 0.61 | 0.61 | 0.59 | 4.89 | 0.0054 | 0.001 | 0.0013 | 0.13 | 0.021 |
| 3591858 | 0.68 | 0 | 1.28 | 0.62 | 0.0011 | 0.58 | 0.65 | 0.63 | 0.6 | 7.39 | 0.0017 | 0.0001 | 0 | 0.02 | 0 |
| 3591985 | 0.6 | 0.0128 | 1.25 | 0.6 | 0.0042 | 0.59 | 0.61 | 0.61 | 0.59 | 5.29 | 0.0034 | 0.0101 | 0.0121 | 0.07 | 0.026 |
| 3592046 | 0.61 | 0.0056 | 1.37 | 0.62 | 0.0007 | 0.64 | 0.6 | 0.63 | 0.62 | 5.82 | 0.0021 | 0.01 | 0.0046 | 0.2 | 0.027 |
| 3592047 | 0.63 | 0.0015 | 1.29 | 0.63 | 0.0002 | 0.7 | 0.56 | 0.62 | 0.65 | 6.69 | 0.0003 | 0.004 | 0.0012 | 0.05 | 0.004 |
| 3592395 | 0.61 | 0.0101 | 1.27 | 0.55 | 0.1438 | 0.42 | 0.68 | 0.58 | 0.53 | 4.01 | 0.1404 | 0.0068 | 0.0123 | 0.05 | 0.011 |
| 3593171 | 0.62 | 0.0029 | 1.64 | 0.62 | 0.0007 | 0.61 | 0.63 | 0.63 | 0.61 | 5.39 | 0.0009 | 0.0019 | 0.0013 | 0.07 | 0.011 |
| 3594101 | 0.61 | 0.0059 | 1.28 | 0.6 | 0.0042 | 0.46 | 0.76 | 0.66 | 0.57 | 4.57 | 0.0026 | 0.026 | 0.0227 | 0.1 | 0.043 |
| 3595811 | 0.63 | 0.0023 | 1.26 | 0.61 | 0.0028 | 0.61 | 0.6 | 0.61 | 0.6 | 4.89 | 0.0018 | 0.0015 | 0.0027 | 0.38 | 0.146 |
| 3595872 | 0.62 | 0.0041 | 1.42 | 0.61 | 0.0028 | 0.6 | 0.61 | 0.62 | 0.6 | 5.13 | 0.0011 | 0.0028 | 0.0033 | 0.04 | 0.011 |
| 3596235 | 0.68 | 0 | 1.28 | 0.63 | 0.0002 | 0.6 | 0.66 | 0.65 | 0.62 | 3.58 | 0.0001 | 0 | 0.0001 | 0.01 | 0.002 |
| 3597406 | 0.61 | 0.0059 | 1.29 | 0.6 | 0.0064 | 0.6 | 0.59 | 0.6 | 0.59 | 6.28 | 0.0055 | 0.0033 | 0.0053 | 0.22 | 0.069 |
| 3597468 | 0.61 | 0.0067 | 1.25 | 0.58 | 0.0276 | 0.59 | 0.56 | 0.58 | 0.57 | 6.2 | 0.0178 | 0.0033 | 0.0069 | 0.06 | 0.029 |
| 3597671 | 0.66 | 0.0001 | 1.32 | 0.6 | 0.0042 | 0.58 | 0.62 | 0.61 | 0.59 | 5.41 | 0.0041 | 0 | 0.0001 | 0.05 | 0.009 |
| 3598717 | 0.63 | 0.0016 | 1.36 | 0.61 | 0.0028 | 0.64 | 0.57 | 0.61 | 0.61 | 6.44 | 0.0032 | 0.0032 | 0.0024 | 0.1 | 0.007 |
| 3599449 | 0.61 | 0.009 | 1.32 | 0.61 | 0.0018 | 0.54 | 0.68 | 0.64 | 0.59 | 4.41 | 0.0019 | 0.0148 | 0.009 | 0.58 | 0.1 |
| 3602509 | 0.65 | 0.0002 | 1.3 | 0.63 | 0.0002 | 0.67 | 0.59 | 0.63 | 0.64 | 5.42 | 0.0003 | 0.0004 | 0.0003 | 0.01 | 0.003 |
| 3605249 | 0.63 | 0.0014 | 1.3 | 0.61 | 0.0028 | 0.65 | 0.56 | 0.61 | 0.61 | 5.41 | 0.0036 | 0.001 | 0.0011 | 0.02 | 0.011 |
| 3606117 | 0.61 | 0.0075 | 1.25 | 0.61 | 0.0018 | 0.5 | 0.73 | 0.66 | 0.59 | 4.63 | 0.0008 | 0.0037 | 0.0055 | 0.09 | 0.101 |
| 3606409 | 0.66 | 0.0001 | 1.43 | 0.63 | 0.0002 | 0.63 | 0.63 | 0.64 | 0.63 | 5.58 | 0.0001 | 0.0001 | 0.0001 | 0.02 | 0.002 |
| 3608201 | 0.62 | 0.0046 | 1.25 | 0.6 | 0.0042 | 0.54 | 0.66 | 0.63 | 0.59 | 5.04 | 0.0027 | 0.0027 | 0.0042 | 0.04 | 0.026 |
| 3609166 | 0.63 | 0.0011 | 1.35 | 0.62 | 0.0011 | 0.59 | 0.64 | 0.63 | 0.61 | 5.15 | 0.0006 | 0.0015 | 0.0011 | 0.04 | 0.004 |
| 3615597 | 0.63 | 0.0016 | 1.26 | 0.62 | 0.0011 | 0.56 | 0.67 | 0.64 | 0.6 | 4.92 | 0.0003 | 0.0023 | 0.0031 | 0.01 | 0.012 |
| 3615604 | 0.63 | 0.0012 | 1.26 | 0.62 | 0.0007 | 0.5 | 0.74 | 0.67 | 0.59 | 6.26 | 0.0004 | 0.0024 | 0.0012 | 0.04 | 0.011 |
| 3615659 | 0.62 | 0.0038 | 1.29 | 0.62 | 0.0011 | 0.58 | 0.65 | 0.63 | 0.6 | 4.57 | 0.0012 | 0.0047 | 0.0039 | 0.05 | 0.023 |
| 3615989 | 0.61 | 0.0062 | 1.26 | 0.59 | 0.0138 | 0.55 | 0.62 | 0.6 | 0.58 | 4.52 | 0.0284 | 0.0038 | 0.0033 | 0.02 | 0.005 |
| 3621014 | 0.66 | 0.0001 | 1.44 | 0.63 | 0.0004 | 0.52 | 0.73 | 0.67 | 0.6 | 5.45 | 0.0001 | 0.0001 | 0.0001 | 0.02 | 0.003 |
| 3623329 | 0.64 | 0.001 | 1.38 | 0.63 | 0.0004 | 0.53 | 0.72 | 0.67 | 0.6 | 5.71 | 0.0002 | 0.0005 | 0.0007 | 0.02 | 0.007 |
| 3624199 | 0.64 | 0.0005 | 1.25 | 0.61 | 0.0018 | 0.54 | 0.68 | 0.64 | 0.59 | 6.12 | 0.0038 | 0.0012 | 0.0009 | 0.02 | 0.006 |
| 3624533 | 0.66 | 0.0001 | 1.33 | 0.63 | 0.0002 | 0.57 | 0.69 | 0.66 | 0.61 | 4.65 | 0.0001 | 0 | 0.0001 | 0 | 0.002 |
| 3624702 | 0.61 | 0.0078 | 1.33 | 0.58 | 0.0197 | 0.52 | 0.64 | 0.6 | 0.57 | 3.93 | 0.0249 | 0.0115 | 0.0083 | 0.08 | 0.014 |
| 3625565 | 0.62 | 0.0026 | 1.32 | 0.59 | 0.0095 | 0.44 | 0.76 | 0.65 | 0.56 | 3.93 | 0.0036 | 0.0079 | 0.0143 | 0.28 | 0.258 |
| 3626570 | 0.63 | 0.0016 | 1.29 | 0.61 | 0.0018 | 0.53 | 0.69 | 0.64 | 0.59 | 4.56 | 0.0007 | 0.0009 | 0.0014 | 0.09 | 0.009 |
| 3626585 | 0.63 | 0.0011 | 1.25 | 0.61 | 0.0018 | 0.58 | 0.64 | 0.63 | 0.6 | 5.82 | 0.0029 | 0.0008 | 0.0009 | 0.05 | 0.005 |
| 3626730 | 0.63 | 0.0016 | 1.44 | 0.62 | 0.0007 | 0.57 | 0.67 | 0.64 | 0.61 | 5.14 | 0.0006 | 0.0014 | 0.0015 | 0.06 | 0.01 |
| 3627045 | 0.64 | 0.0008 | 1.25 | 0.6 | 0.0064 | 0.49 | 0.71 | 0.64 | 0.57 | 5.25 | 0.001 | 0.0014 | 0.0018 | 0.01 | 0.011 |
| 3628011 | 0.61 | 0.0101 | 1.25 | 0.58 | 0.0197 | 0.47 | 0.7 | 0.62 | 0.56 | 4.75 | 0.0188 | 0.0111 | 0.0146 | 0.21 | 0.09 |
| 3628052 | 0.61 | 0.0059 | 1.25 | 0.59 | 0.0138 | 0.5 | 0.67 | 0.61 | 0.57 | 5.4 | 0.0128 | 0.006 | 0.0068 | 0.23 | 0.053 |
| 3628057 | 0.64 | 0.0006 | 1.26 | 0.62 | 0.0007 | 0.48 | 0.78 | 0.69 | 0.59 | 5.07 | 0.0002 | 0.0014 | 0.0008 | 0.05 | 0.007 |
| 3628471 | 0.64 | 0.0005 | 1.45 | 0.61 | 0.0018 | 0.6 | 0.62 | 0.62 | 0.6 | 6.06 | 0.0036 | 0.0008 | 0.0005 | 0.02 | 0.002 |
| 3628924 | 0.64 | 0.0007 | 1.31 | 0.58 | 0.0276 | 0.48 | 0.68 | 0.61 | 0.56 | 4.63 | 0.0344 | 0.0026 | 0.002 | 0.02 | 0.006 |
| 3630191 | 0.64 | 0.0006 | 1.38 | 0.64 | 0.0001 | 0.61 | 0.67 | 0.66 | 0.63 | 6.32 | 0.0001 | 0.0003 | 0.0005 | 0.02 | 0.002 |
| 3631414 | 0.65 | 0.0004 | 1.27 | 0.63 | 0.0002 | 0.45 | 0.83 | 0.73 | 0.59 | 4.2 | 0 | 0.0001 | 0.0005 | 0 | 0.002 |
| 3632170 | 0.6 | 0.0111 | 1.41 | 0.6 | 0.0064 | 0.63 | 0.56 | 0.6 | 0.6 | 6.09 | 0.0067 | 0.0096 | 0.0083 | 0.04 | 0.007 |
| 3633110 | 0.63 | 0.0012 | 1.37 | 0.58 | 0.0276 | 0.53 | 0.62 | 0.59 | 0.56 | 4.04 | 0.0305 | 0.0037 | 0.003 | 0.05 | 0.012 |
| 3633223 | 0.61 | 0.006 | 1.27 | 0.6 | 0.0042 | 0.66 | 0.54 | 0.6 | 0.61 | 6.33 | 0.0043 | 0.0053 | 0.0044 | 0.09 | 0.016 |
| 3633226 | 0.62 | 0.0024 | 1.41 | 0.6 | 0.0064 | 0.51 | 0.68 | 0.63 | 0.58 | 4.11 | 0.0033 | 0.0009 | 0.0018 | 0.01 | 0.009 |
| 3634074 | 0.62 | 0.0027 | 1.42 | 0.62 | 0.0011 | 0.64 | 0.59 | 0.62 | 0.62 | 5.31 | 0.0012 | 0.0034 | 0.0022 | 0.17 | 0.014 |
| 3643958 | 0.61 | 0.0075 | 1.32 | 0.59 | 0.0138 | 0.55 | 0.62 | 0.6 | 0.58 | 5.77 | 0.0187 | 0.0111 | 0.0076 | 0.22 | 0.024 |
| 3645267 | 0.64 | 0.0004 | 1.3 | 0.61 | 0.0018 | 0.65 | 0.57 | 0.61 | 0.62 | 7.04 | 0.0013 | 0.0004 | 0.0004 | 0.01 | 0.001 |
| 3645314 | 0.63 | 0.0022 | 1.34 | 0.61 | 0.0028 | 0.7 | 0.51 | 0.6 | 0.63 | 5.55 | 0.0041 | 0.0019 | 0.0017 | 0.02 | 0.005 |
| 3645591 | 0.63 | 0.0013 | 1.28 | 0.59 | 0.0095 | 0.59 | 0.59 | 0.6 | 0.59 | 4.87 | 0.0096 | 0.0049 | 0.0036 | 0.15 | 0.032 |
| 3647535 | 0.62 | 0.0031 | 1.31 | 0.61 | 0.0028 | 0.58 | 0.63 | 0.62 | 0.6 | 5.33 | 0.0025 | 0.0039 | 0.0041 | 0.1 | 0.02 |
| 3648373 | 0.62 | 0.0032 | 1.26 | 0.58 | 0.0276 | 0.5 | 0.65 | 0.6 | 0.56 | 4.62 | 0.0323 | 0.0045 | 0.0067 | 0.12 | 0.02 |
| 3651472 | 0.64 | 0.0008 | 1.26 | 0.61 | 0.0018 | 0.55 | 0.67 | 0.64 | 0.59 | 5.07 | 0.0009 | 0.0015 | 0.0015 | 0.03 | 0.005 |
| 3653349 | 0.63 | 0.0018 | 1.29 | 0.6 | 0.0042 | 0.55 | 0.65 | 0.62 | 0.59 | 4.92 | 0.002 | 0.0025 | 0.0042 | 0.1 | 0.068 |
| 3653673 | 0.63 | 0.0012 | 1.37 | 0.64 | 0.0001 | 0.65 | 0.62 | 0.64 | 0.64 | 6.32 | 0.0001 | 0.0011 | 0.0008 | 0.01 | 0.001 |
| 3655101 | 0.61 | 0.0092 | 1.56 | 0.61 | 0.0028 | 0.55 | 0.66 | 0.63 | 0.59 | 4.47 | 0.0037 | 0.0034 | 0.0039 | 0.09 | 0.026 |
| 3658610 | 0.66 | 0.0001 | 1.31 | 0.63 | 0.0004 | 0.54 | 0.71 | 0.66 | 0.6 | 5.67 | 0.0002 | 0 | 0.0001 | 0.01 | 0.001 |
| 3659201 | 0.6 | 0.0133 | 1.35 | 0.58 | 0.0276 | 0.51 | 0.64 | 0.6 | 0.56 | 5.34 | 0.0158 | 0.0172 | 0.0231 | 0.23 | 0.038 |
| 3659319 | 0.66 | 0.0001 | 1.46 | 0.64 | 0.0001 | 0.58 | 0.69 | 0.66 | 0.62 | 5.09 | 0.0002 | 0.0003 | 0.0002 | 0.03 | 0.003 |
| 3660917 | 0.62 | 0.005 | 1.26 | 0.59 | 0.0095 | 0.55 | 0.63 | 0.61 | 0.58 | 3.05 | 0.013 | 0.0215 | 0.0106 | 0.28 | 0.04 |
| 3660927 | 0.6 | 0.0107 | 1.26 | 0.57 | 0.0513 | 0.43 | 0.71 | 0.61 | 0.55 | 4.12 | 0.0105 | 0.0094 | 0.0164 | 0.13 | 0.091 |
| 3665635 | 0.61 | 0.0057 | 1.41 | 0.59 | 0.0138 | 0.61 | 0.56 | 0.59 | 0.59 | 5.24 | 0.0062 | 0.0036 | 0.0042 | 0.05 | 0.009 |
| 3666111 | 0.64 | 0.0006 | 1.27 | 0.63 | 0.0002 | 0.49 | 0.79 | 0.7 | 0.6 | 4.28 | 0.0001 | 0.0011 | 0.0009 | 0.02 | 0.004 |
| 3666869 | 0.64 | 0.0006 | 1.64 | 0.64 | 0.0001 | 0.56 | 0.71 | 0.67 | 0.61 | 4.89 | 0.0001 | 0.0004 | 0.0004 | 0.08 | 0.014 |
| 3668992 | 0.61 | 0.0051 | 1.3 | 0.6 | 0.0064 | 0.53 | 0.66 | 0.62 | 0.58 | 4.44 | 0.0017 | 0.0021 | 0.0043 | 0.1 | 0.046 |
| 3671113 | 0.62 | 0.004 | 1.28 | 0.57 | 0.0513 | 0.41 | 0.73 | 0.61 | 0.55 | 4.96 | 0.029 | 0.0021 | 0.0071 | 0.02 | 0.016 |
| 3677863 | 0.64 | 0.0005 | 1.27 | 0.61 | 0.0028 | 0.52 | 0.69 | 0.64 | 0.59 | 5.25 | 0.0045 | 0.0033 | 0.0019 | 0.08 | 0.016 |
| 3679601 | 0.62 | 0.0027 | 1.28 | 0.58 | 0.0276 | 0.5 | 0.66 | 0.6 | 0.56 | 4.47 | 0.0241 | 0.0027 | 0.0045 | 0.06 | 0.023 |
| 3683054 | 0.62 | 0.0028 | 1.53 | 0.63 | 0.0004 | 0.68 | 0.57 | 0.62 | 0.64 | 5.14 | 0.0004 | 0.0014 | 0.0014 | 0.11 | 0.01 |
| 3685080 | 0.61 | 0.0075 | 1.29 | 0.58 | 0.0276 | 0.49 | 0.67 | 0.6 | 0.56 | 3.73 | 0.0218 | 0.0017 | 0.0044 | 0.1 | 0.05 |
| 3687262 | 0.63 | 0.0014 | 1.27 | 0.63 | 0.0004 | 0.71 | 0.54 | 0.62 | 0.65 | 8.12 | 0.0006 | 0.0018 | 0.0013 | 0.09 | 0.008 |
| 3687792 | 0.62 | 0.0033 | 1.5 | 0.6 | 0.0042 | 0.57 | 0.63 | 0.62 | 0.59 | 5.65 | 0.0075 | 0.0026 | 0.0022 | 0.1 | 0.012 |
| 3690089 | 0.62 | 0.0026 | 1.41 | 0.58 | 0.0197 | 0.55 | 0.61 | 0.6 | 0.57 | 5.33 | 0.0218 | 0.0042 | 0.0027 | 0.1 | 0.006 |
| 3692857 | 0.61 | 0.0071 | 1.28 | 0.59 | 0.0138 | 0.54 | 0.63 | 0.6 | 0.57 | 5.34 | 0.0126 | 0.0105 | 0.007 | 0.38 | 0.066 |
| 3693546 | 0.62 | 0.005 | 1.31 | 0.57 | 0.038 | 0.58 | 0.56 | 0.58 | 0.57 | 5.36 | 0.0226 | 0.0063 | 0.0072 | 0.11 | 0.053 |
| 3693711 | 0.62 | 0.0025 | 1.35 | 0.59 | 0.0138 | 0.52 | 0.65 | 0.61 | 0.57 | 4.97 | 0.0113 | 0.003 | 0.0038 | 0.07 | 0.015 |
| 3693737 | 0.62 | 0.0031 | 1.26 | 0.65 | 0 | 0.65 | 0.64 | 0.65 | 0.64 | 7.19 | 0.0001 | 0.004 | 0.0026 | 0.18 | 0.038 |
| 3693746 | 0.64 | 0.0009 | 1.29 | 0.56 | 0.0889 | 0.36 | 0.77 | 0.61 | 0.54 | 5.2 | 0.0493 | 0.0037 | 0.0064 | 0.18 | 0.113 |
| 3696670 | 0.62 | 0.005 | 1.35 | 0.62 | 0.0007 | 0.55 | 0.69 | 0.65 | 0.6 | 5.01 | 0.0007 | 0.0069 | 0.0065 | 0.17 | 0.028 |
| 3697925 | 0.63 | 0.0021 | 1.27 | 0.59 | 0.0095 | 0.52 | 0.66 | 0.62 | 0.58 | 4.75 | 0.0147 | 0.0013 | 0.0017 | 0.21 | 0.033 |
| 3699559 | 0.64 | 0.0008 | 1.26 | 0.62 | 0.0011 | 0.56 | 0.67 | 0.64 | 0.6 | 4.82 | 0.0006 | 0.0008 | 0.0011 | 0.03 | 0.009 |
| 3701588 | 0.64 | 0.0008 | 1.26 | 0.6 | 0.0064 | 0.59 | 0.6 | 0.61 | 0.59 | 5.75 | 0.0205 | 0.0051 | 0.0014 | 0.09 | 0.006 |
| 3705531 | 0.61 | 0.0094 | 1.37 | 0.57 | 0.038 | 0.42 | 0.73 | 0.62 | 0.55 | 4.01 | 0.0426 | 0.0248 | 0.02 | 0.18 | 0.017 |
| 3707211 | 0.62 | 0.0046 | 1.43 | 0.6 | 0.0064 | 0.56 | 0.63 | 0.61 | 0.58 | 5.42 | 0.0079 | 0.0084 | 0.0048 | 0.07 | 0.009 |
| 3708006 | 0.6 | 0.0126 | 1.37 | 0.62 | 0.0011 | 0.66 | 0.57 | 0.61 | 0.62 | 6.34 | 0.0019 | 0.0052 | 0.0044 | 0.09 | 0.014 |
| 3709275 | 0.62 | 0.0033 | 1.29 | 0.59 | 0.0138 | 0.52 | 0.65 | 0.61 | 0.57 | 5.13 | 0.0077 | 0.0019 | 0.0031 | 0.09 | 0.023 |
| 3710735 | 0.65 | 0.0004 | 1.29 | 0.63 | 0.0002 | 0.66 | 0.6 | 0.63 | 0.63 | 4.04 | 0.0006 | 0.0016 | 0.0008 | 0.14 | 0.021 |
| 3714102 | 0.62 | 0.0038 | 1.28 | 0.58 | 0.0197 | 0.57 | 0.59 | 0.59 | 0.57 | 5 | 0.0398 | 0.0065 | 0.0033 | 0.18 | 0.011 |
| 3715142 | 0.66 | 0.0002 | 1.37 | 0.63 | 0.0004 | 0.61 | 0.64 | 0.64 | 0.62 | 5.39 | 0.0006 | 0.0001 | 0.0002 | 0.09 | 0.012 |
| 3715434 | 0.61 | 0.0101 | 1.26 | 0.59 | 0.0138 | 0.54 | 0.63 | 0.6 | 0.57 | 4.69 | 0.0183 | 0.0153 | 0.0116 | 0.2 | 0.07 |
| 3716021 | 0.63 | 0.0022 | 1.36 | 0.61 | 0.0018 | 0.61 | 0.61 | 0.62 | 0.61 | 4.77 | 0.002 | 0.0014 | 0.002 | 0.13 | 0.022 |
| 3716674 | 0.6 | 0.0113 | 1.26 | 0.59 | 0.0138 | 0.56 | 0.61 | 0.6 | 0.58 | 5.17 | 0.0244 | 0.022 | 0.0142 | 0.41 | 0.084 |
| 3716757 | 0.64 | 0.0009 | 1.28 | 0.65 | 0 | 0.62 | 0.68 | 0.67 | 0.64 | 5.39 | 0 | 0.0016 | 0.0019 | 0.03 | 0.004 |
| 3717647 | 0.67 | 0 | 1.27 | 0.65 | 0 | 0.54 | 0.76 | 0.7 | 0.62 | 5.34 | 0 | 0.0003 | 0.0002 | 0.07 | 0.004 |
| 3717681 | 0.62 | 0.0038 | 1.31 | 0.62 | 0.0007 | 0.55 | 0.69 | 0.65 | 0.6 | 5.48 | 0.0008 | 0.0033 | 0.0033 | 0.31 | 0.099 |
| 3717684 | 0.63 | 0.001 | 1.28 | 0.64 | 0.0001 | 0.6 | 0.68 | 0.66 | 0.63 | 6.96 | 0.0001 | 0.0019 | 0.001 | 0.13 | 0.008 |
| 3718837 | 0.63 | 0.0014 | 1.26 | 0.6 | 0.0042 | 0.78 | 0.42 | 0.58 | 0.65 | 6.39 | 0.0051 | 0.0033 | 0.0026 | 0.06 | 0.004 |
| 3719123 | 0.61 | 0.0091 | 1.68 | 0.59 | 0.0138 | 0.5 | 0.67 | 0.61 | 0.57 | 4.59 | 0.0069 | 0.007 | 0.0075 | 0.19 | 0.028 |
| 3720778 | 0.62 | 0.0033 | 1.33 | 0.63 | 0.0002 | 0.57 | 0.69 | 0.66 | 0.61 | 5.66 | 0.0002 | 0.0041 | 0.0028 | 0.23 | 0.034 |
| 3720990 | 0.63 | 0.0011 | 1.27 | 0.6 | 0.0042 | 0.54 | 0.66 | 0.63 | 0.59 | 7 | 0.0022 | 0.0013 | 0.0024 | 0.04 | 0.008 |
| 3722990 | 0.63 | 0.0013 | 1.27 | 0.61 | 0.0018 | 0.51 | 0.71 | 0.65 | 0.59 | 4 | 0.0018 | 0.0025 | 0.002 | 0.03 | 0.003 |
| 3723305 | 0.62 | 0.0029 | 1.39 | 0.59 | 0.0138 | 0.61 | 0.56 | 0.59 | 0.59 | 5.33 | 0.0215 | 0.0039 | 0.0029 | 0.07 | 0.014 |
| 3726811 | 0.63 | 0.0016 | 1.46 | 0.59 | 0.0138 | 0.52 | 0.65 | 0.61 | 0.57 | 4.63 | 0.0154 | 0.0035 | 0.0028 | 0.26 | 0.038 |
| 3728316 | 0.64 | 0.0005 | 1.26 | 0.62 | 0.0007 | 0.58 | 0.66 | 0.64 | 0.61 | 5.83 | 0.0016 | 0.0017 | 0.0005 | 0.26 | 0.009 |
| 3729194 | 0.62 | 0.003 | 1.33 | 0.59 | 0.0095 | 0.54 | 0.64 | 0.61 | 0.58 | 4.39 | 0.0188 | 0.0049 | 0.0046 | 0.08 | 0.016 |
| 3729227 | 0.63 | 0.0023 | 1.35 | 0.62 | 0.0011 | 0.58 | 0.65 | 0.63 | 0.6 | 6.23 | 0.0018 | 0.003 | 0.0022 | 0.03 | 0.005 |
| 3732475 | 0.65 | 0.0002 | 1.39 | 0.63 | 0.0004 | 0.56 | 0.69 | 0.66 | 0.61 | 5.12 | 0.0003 | 0.0004 | 0.0003 | 0.03 | 0.003 |
| 3732477 | 0.62 | 0.0024 | 1.27 | 0.62 | 0.0007 | 0.55 | 0.69 | 0.65 | 0.6 | 4.33 | 0.0002 | 0.0017 | 0.004 | 0.07 | 0.024 |
| 3732479 | 0.63 | 0.0019 | 1.27 | 0.61 | 0.0028 | 0.53 | 0.68 | 0.64 | 0.59 | 6.12 | 0.0033 | 0.0027 | 0.002 | 0.22 | 0.023 |
| 3732480 | 0.67 | 0 | 1.35 | 0.61 | 0.0018 | 0.51 | 0.71 | 0.65 | 0.59 | 3.5 | 0.0014 | 0.0002 | 0.0002 | 0.03 | 0.001 |
| 3734754 | 0.63 | 0.0016 | 1.62 | 0.6 | 0.0042 | 0.64 | 0.56 | 0.6 | 0.6 | 5.01 | 0.0037 | 0.0015 | 0.001 | 0.01 | 0.002 |
| 3734863 | 0.61 | 0.0056 | 1.38 | 0.58 | 0.0276 | 0.55 | 0.6 | 0.59 | 0.57 | 5.95 | 0.021 | 0.0037 | 0.0053 | 0.04 | 0.016 |
| 3737304 | 0.63 | 0.002 | 1.3 | 0.63 | 0.0004 | 0.54 | 0.71 | 0.66 | 0.6 | 5.7 | 0.0003 | 0.0059 | 0.0043 | 0.13 | 0.013 |
| 3737985 | 0.61 | 0.0053 | 1.28 | 0.62 | 0.0011 | 0.64 | 0.59 | 0.62 | 0.62 | 6.8 | 0.0015 | 0.0049 | 0.0044 | 0.08 | 0.014 |
| 3738300 | 0.61 | 0.0064 | 1.29 | 0.62 | 0.0011 | 0.68 | 0.55 | 0.61 | 0.63 | 6.91 | 0.0018 | 0.0069 | 0.004 | 0.06 | 0.007 |
| 3739125 | 0.63 | 0.0012 | 1.56 | 0.61 | 0.0028 | 0.55 | 0.66 | 0.63 | 0.59 | 4.25 | 0.0031 | 0.0042 | 0.0026 | 0.07 | 0.016 |
| 3739523 | 0.63 | 0.0014 | 1.39 | 0.6 | 0.0064 | 0.5 | 0.7 | 0.63 | 0.58 | 5.3 | 0.0048 | 0.0024 | 0.0017 | 0.26 | 0.064 |
| 3740129 | 0.64 | 0.0008 | 1.46 | 0.63 | 0.0004 | 0.65 | 0.6 | 0.63 | 0.63 | 5.47 | 0.0004 | 0.0007 | 0.0006 | 0 | 0.001 |
| 3740130 | 0.63 | 0.002 | 1.58 | 0.61 | 0.0028 | 0.57 | 0.64 | 0.62 | 0.59 | 5.15 | 0.002 | 0.0026 | 0.0024 | 0.03 | 0.007 |
| 3744423 | 0.61 | 0.0064 | 1.3 | 0.63 | 0.0002 | 0.67 | 0.59 | 0.63 | 0.64 | 7.58 | 0.0002 | 0.0092 | 0.0084 | 0.23 | 0.029 |
| 3746953 | 0.61 | 0.006 | 1.27 | 0.57 | 0.038 | 0.45 | 0.7 | 0.61 | 0.55 | 4.11 | 0.0237 | 0.0041 | 0.0099 | 0.12 | 0.047 |
| 3746967 | 0.6 | 0.0135 | 1.3 | 0.59 | 0.0095 | 0.48 | 0.71 | 0.63 | 0.57 | 5.09 | 0.0068 | 0.0232 | 0.0125 | 0.08 | 0.016 |
| 3747223 | 0.64 | 0.0006 | 1.38 | 0.66 | 0 | 0.6 | 0.71 | 0.69 | 0.64 | 6.51 | 0 | 0.0006 | 0.0005 | 0.03 | 0.002 |
| 3748953 | 0.6 | 0.0133 | 1.32 | 0.58 | 0.0276 | 0.56 | 0.59 | 0.59 | 0.57 | 4.8 | 0.0439 | 0.0129 | 0.0097 | 0.11 | 0.019 |
| 3750786 | 0.61 | 0.0093 | 1.35 | 0.62 | 0.0011 | 0.68 | 0.55 | 0.61 | 0.63 | 5.19 | 0.001 | 0.0102 | 0.007 | 0.11 | 0.011 |
| 3752711 | 0.6 | 0.0111 | 1.34 | 0.61 | 0.0028 | 0.7 | 0.51 | 0.6 | 0.63 | 5.53 | 0.0019 | 0.0084 | 0.0089 | 0.04 | 0.009 |
| 3754530 | 0.64 | 0.0005 | 1.32 | 0.62 | 0.0011 | 0.59 | 0.64 | 0.63 | 0.61 | 4.86 | 0.0006 | 0.0003 | 0.0006 | 0.01 | 0.003 |
| 3754741 | 0.6 | 0.0117 | 1.29 | 0.6 | 0.0064 | 0.56 | 0.63 | 0.61 | 0.58 | 4.08 | 0.0102 | 0.0456 | 0.0305 | 0.13 | 0.023 |
| 3756204 | 0.61 | 0.0078 | 1.25 | 0.6 | 0.0064 | 0.54 | 0.65 | 0.62 | 0.58 | 6.24 | 0.0028 | 0.006 | 0.0078 | 0.05 | 0.013 |
| 3759590 | 0.62 | 0.0027 | 1.29 | 0.59 | 0.0138 | 0.52 | 0.65 | 0.61 | 0.57 | 4.14 | 0.0165 | 0.0052 | 0.0048 | 0.12 | 0.032 |
| 3760197 | 0.62 | 0.0029 | 1.32 | 0.57 | 0.0513 | 0.6 | 0.53 | 0.57 | 0.57 | 4.46 | 0.0636 | 0.0068 | 0.004 | 0.21 | 0.022 |
| 3761752 | 0.63 | 0.002 | 1.26 | 0.6 | 0.0064 | 0.54 | 0.65 | 0.62 | 0.58 | 4.7 | 0.0225 | 0.0101 | 0.0028 | 0.48 | 0.041 |
| 3762575 | 0.64 | 0.0006 | 1.42 | 0.58 | 0.0197 | 0.55 | 0.61 | 0.6 | 0.57 | 5.23 | 0.0256 | 0.0013 | 0.001 | 0.06 | 0.015 |
| 3764121 | 0.63 | 0.0014 | 1.32 | 0.61 | 0.0028 | 0.64 | 0.57 | 0.61 | 0.61 | 6.19 | 0.0044 | 0.0018 | 0.0014 | 0.04 | 0.004 |
| 3764125 | 0.63 | 0.0019 | 1.26 | 0.62 | 0.0007 | 0.62 | 0.62 | 0.63 | 0.62 | 5.95 | 0.0005 | 0.0012 | 0.0012 | 0.03 | 0.004 |
| 3764127 | 0.62 | 0.0032 | 1.27 | 0.62 | 0.0011 | 0.55 | 0.68 | 0.64 | 0.6 | 4.63 | 0.0011 | 0.012 | 0.0069 | 0.29 | 0.106 |
| 3764884 | 0.61 | 0.0064 | 1.32 | 0.61 | 0.0028 | 0.61 | 0.6 | 0.61 | 0.6 | 5.36 | 0.0015 | 0.0062 | 0.0052 | 0.1 | 0.028 |
| 3764921 | 0.64 | 0.0007 | 1.4 | 0.6 | 0.0064 | 0.56 | 0.63 | 0.61 | 0.58 | 6.37 | 0.0159 | 0.0011 | 0.0008 | 0.04 | 0.005 |
| 3765738 | 0.61 | 0.0062 | 1.34 | 0.62 | 0.0011 | 0.53 | 0.7 | 0.65 | 0.59 | 4.6 | 0.0005 | 0.0068 | 0.0086 | 0.07 | 0.045 |
| 3765761 | 0.62 | 0.0042 | 1.25 | 0.61 | 0.0028 | 0.6 | 0.61 | 0.62 | 0.6 | 5.63 | 0.0021 | 0.0031 | 0.0039 | 0.25 | 0.083 |
| 3765767 | 0.61 | 0.008 | 1.31 | 0.58 | 0.0197 | 0.5 | 0.66 | 0.61 | 0.57 | 4 | 0.0195 | 0.0193 | 0.0184 | 0.2 | 0.141 |
| 3766896 | 0.61 | 0.0083 | 1.29 | 0.58 | 0.0276 | 0.5 | 0.66 | 0.6 | 0.56 | 4.94 | 0.0307 | 0.0178 | 0.0118 | 0.16 | 0.063 |
| 3768039 | 0.65 | 0.0003 | 1.35 | 0.62 | 0.0007 | 0.5 | 0.76 | 0.68 | 0.59 | 4.62 | 0.0001 | 0.001 | 0.0015 | 0.03 | 0.019 |
| 3768121 | 0.63 | 0.0014 | 1.25 | 0.6 | 0.0042 | 0.52 | 0.68 | 0.63 | 0.58 | 4.98 | 0.0029 | 0.0047 | 0.0035 | 0.07 | 0.031 |
| 3768258 | 0.63 | 0.0012 | 1.29 | 0.61 | 0.0018 | 0.59 | 0.63 | 0.63 | 0.6 | 4.37 | 0.0012 | 0.0029 | 0.002 | 0.16 | 0.029 |
| 3769780 | 0.63 | 0.0022 | 1.26 | 0.63 | 0.0004 | 0.59 | 0.66 | 0.65 | 0.61 | 6.27 | 0.0003 | 0.0029 | 0.002 | 0.11 | 0.015 |
| 3770244 | 0.62 | 0.0032 | 1.25 | 0.62 | 0.0007 | 0.53 | 0.71 | 0.66 | 0.6 | 8.23 | 0.0003 | 0.0023 | 0.0025 | 0.14 | 0.02 |
| 3770564 | 0.62 | 0.0028 | 1.36 | 0.62 | 0.0011 | 0.66 | 0.57 | 0.61 | 0.62 | 6.2 | 0.0008 | 0.0027 | 0.0023 | 0.06 | 0.015 |
| 3770565 | 0.6 | 0.0114 | 1.31 | 0.59 | 0.0095 | 0.56 | 0.62 | 0.61 | 0.58 | 6.42 | 0.006 | 0.0092 | 0.0084 | 0.17 | 0.041 |
| 3770746 | 0.62 | 0.0051 | 1.35 | 0.59 | 0.0095 | 0.57 | 0.61 | 0.6 | 0.58 | 6.91 | 0.0092 | 0.009 | 0.0056 | 0.11 | 0.019 |
| 3771039 | 0.61 | 0.0076 | 1.28 | 0.6 | 0.0064 | 0.48 | 0.72 | 0.64 | 0.57 | 5.11 | 0.0024 | 0.0074 | 0.0087 | 0.16 | 0.057 |
| 3771094 | 0.63 | 0.0022 | 1.43 | 0.6 | 0.0042 | 0.52 | 0.68 | 0.63 | 0.58 | 4.09 | 0.0056 | 0.0064 | 0.0027 | 0.12 | 0.015 |
| 3771337 | 0.61 | 0.0057 | 1.64 | 0.61 | 0.0018 | 0.59 | 0.63 | 0.63 | 0.6 | 4.64 | 0.0025 | 0.0081 | 0.0041 | 0.15 | 0.015 |
| 3776189 | 0.65 | 0.0002 | 1.36 | 0.63 | 0.0002 | 0.56 | 0.7 | 0.66 | 0.61 | 6.15 | 0.0001 | 0.0007 | 0.0004 | 0.01 | 0.002 |
| 3776219 | 0.6 | 0.0133 | 1.25 | 0.6 | 0.0064 | 0.49 | 0.71 | 0.64 | 0.57 | 4.26 | 0.0042 | 0.0223 | 0.0174 | 0.1 | 0.045 |
| 3776236 | 0.63 | 0.0014 | 1.29 | 0.58 | 0.0276 | 0.39 | 0.78 | 0.64 | 0.55 | 3.87 | 0.0368 | 0.0045 | 0.0024 | 0.04 | 0.014 |
| 3776446 | 0.61 | 0.0062 | 1.49 | 0.59 | 0.0138 | 0.53 | 0.64 | 0.61 | 0.57 | 4.89 | 0.0141 | 0.0091 | 0.0076 | 0.08 | 0.014 |
| 3776461 | 0.62 | 0.0027 | 1.25 | 0.6 | 0.0064 | 0.58 | 0.61 | 0.61 | 0.59 | 6.29 | 0.0082 | 0.0052 | 0.0035 | 0.03 | 0.005 |
| 3778266 | 0.66 | 0.0001 | 1.29 | 0.61 | 0.0018 | 0.57 | 0.65 | 0.63 | 0.6 | 6.28 | 0.0024 | 0.0004 | 0.0002 | 0.03 | 0.003 |
| 3778629 | 0.65 | 0.0003 | 1.41 | 0.64 | 0.0001 | 0.58 | 0.7 | 0.67 | 0.62 | 6.43 | 0.0001 | 0.0003 | 0.0003 | 0.04 | 0.003 |
| 3778630 | 0.63 | 0.0011 | 1.34 | 0.59 | 0.0138 | 0.63 | 0.54 | 0.59 | 0.59 | 6.04 | 0.0224 | 0.002 | 0.0011 | 0.09 | 0.012 |
| 3779850 | 0.62 | 0.0026 | 1.27 | 0.55 | 0.114 | 0.39 | 0.72 | 0.59 | 0.53 | 4.22 | 0.1004 | 0.007 | 0.007 | 0.1 | 0.04 |
| 3781686 | 0.6 | 0.0122 | 1.26 | 0.55 | 0.1438 | 0.42 | 0.68 | 0.58 | 0.53 | 3.38 | 0.0685 | 0.0308 | 0.0676 | 0.06 | 0.07 |
| 3781689 | 0.63 | 0.0012 | 1.29 | 0.59 | 0.0138 | 0.53 | 0.64 | 0.61 | 0.57 | 5.5 | 0.0105 | 0.0012 | 0.0018 | 0.01 | 0.003 |
| 3783364 | 0.61 | 0.0058 | 1.26 | 0.58 | 0.0197 | 0.56 | 0.6 | 0.59 | 0.57 | 6.36 | 0.0142 | 0.0052 | 0.0072 | 0.02 | 0.008 |
| 3787934 | 0.66 | 0.0001 | 1.31 | 0.64 | 0.0001 | 0.57 | 0.7 | 0.67 | 0.62 | 6.26 | 0.0001 | 0 | 0 | 0 | 0.001 |
| 3789817 | 0.62 | 0.004 | 1.34 | 0.59 | 0.0095 | 0.5 | 0.68 | 0.62 | 0.57 | 5.3 | 0.0081 | 0.0079 | 0.0062 | 0.12 | 0.012 |
| 3795442 | 0.64 | 0.0007 | 1.27 | 0.58 | 0.0197 | 0.49 | 0.68 | 0.61 | 0.56 | 4.08 | 0.0088 | 0.0003 | 0.0014 | 0.02 | 0.046 |
| 3796554 | 0.61 | 0.0067 | 1.3 | 0.57 | 0.038 | 0.44 | 0.71 | 0.61 | 0.55 | 4.53 | 0.0236 | 0.019 | 0.0175 | 0.18 | 0.093 |
| 3798470 | 0.67 | 0.0001 | 1.42 | 0.63 | 0.0004 | 0.58 | 0.67 | 0.65 | 0.61 | 5.95 | 0.0004 | 0.0001 | 0.0001 | 0.01 | 0.001 |
| 3800623 | 0.61 | 0.0083 | 1.25 | 0.59 | 0.0138 | 0.56 | 0.61 | 0.6 | 0.58 | 4.9 | 0.0186 | 0.0126 | 0.0094 | 0.5 | 0.12 |
| 3804170 | 0.64 | 0.0005 | 1.3 | 0.58 | 0.0197 | 0.54 | 0.62 | 0.6 | 0.57 | 4.46 | 0.0214 | 0.0009 | 0.0008 | 0.03 | 0.002 |
| 3804202 | 0.63 | 0.0012 | 1.3 | 0.62 | 0.0011 | 0.56 | 0.67 | 0.64 | 0.6 | 4.59 | 0.001 | 0.0036 | 0.0023 | 0.05 | 0.018 |
| 3806255 | 0.64 | 0.0006 | 1.31 | 0.62 | 0.0007 | 0.64 | 0.6 | 0.63 | 0.62 | 6.72 | 0.0012 | 0.001 | 0.0007 | 0.06 | 0.005 |
| 3806256 | 0.61 | 0.0078 | 1.27 | 0.59 | 0.0138 | 0.48 | 0.7 | 0.62 | 0.57 | 4.44 | 0.021 | 0.0108 | 0.0129 | 0.05 | 0.019 |
| 3807476 | 0.61 | 0.0059 | 1.29 | 0.61 | 0.0018 | 0.52 | 0.7 | 0.65 | 0.59 | 4.95 | 0.0012 | 0.0046 | 0.0056 | 0.15 | 0.039 |
| 3807570 | 0.63 | 0.0013 | 1.34 | 0.62 | 0.0007 | 0.52 | 0.72 | 0.66 | 0.6 | 4.16 | 0.0002 | 0.0001 | 0.0005 | 0.1 | 0.054 |
| 3807629 | 0.65 | 0.0002 | 1.36 | 0.62 | 0.0007 | 0.5 | 0.76 | 0.68 | 0.59 | 4.56 | 0.0001 | 0 | 0.0002 | 0.01 | 0.011 |
| 3808602 | 0.64 | 0.0007 | 1.39 | 0.63 | 0.0004 | 0.63 | 0.62 | 0.63 | 0.62 | 7.24 | 0.0006 | 0.001 | 0.0006 | 0.02 | 0.001 |
| 3809343 | 0.62 | 0.003 | 1.36 | 0.61 | 0.0028 | 0.65 | 0.56 | 0.61 | 0.61 | 5.43 | 0.003 | 0.0024 | 0.0022 | 0.04 | 0.008 |
| 3809673 | 0.64 | 0.0008 | 1.36 | 0.62 | 0.0007 | 0.65 | 0.59 | 0.62 | 0.62 | 6.29 | 0.0006 | 0.0012 | 0.0009 | 0.03 | 0.004 |
| 3809834 | 0.67 | 0.0001 | 1.26 | 0.62 | 0.0011 | 0.5 | 0.73 | 0.66 | 0.59 | 5.07 | 0.0026 | 0.0004 | 0.0002 | 0.01 | 0.002 |
| 3813298 | 0.65 | 0.0003 | 1.3 | 0.59 | 0.0095 | 0.52 | 0.66 | 0.62 | 0.58 | 5.9 | 0.0084 | 0.0003 | 0.0005 | 0.06 | 0.009 |
| 3813299 | 0.62 | 0.0024 | 1.27 | 0.57 | 0.0513 | 0.44 | 0.7 | 0.6 | 0.55 | 4.8 | 0.0317 | 0.0022 | 0.0051 | 0.23 | 0.094 |
| 3815669 | 0.61 | 0.0079 | 1.35 | 0.58 | 0.0197 | 0.56 | 0.6 | 0.59 | 0.57 | 4.76 | 0.0292 | 0.0071 | 0.0064 | 0.08 | 0.016 |
| 3816402 | 0.6 | 0.0146 | 1.38 | 0.61 | 0.0028 | 0.57 | 0.64 | 0.62 | 0.59 | 4.49 | 0.0023 | 0.0122 | 0.0123 | 0.06 | 0.022 |
| 3820658 | 0.62 | 0.0024 | 1.29 | 0.61 | 0.0018 | 0.73 | 0.49 | 0.6 | 0.64 | 6.03 | 0.0031 | 0.0037 | 0.0027 | 0.07 | 0.012 |
| 3820705 | 0.61 | 0.0061 | 1.25 | 0.58 | 0.0197 | 0.6 | 0.56 | 0.59 | 0.58 | 6.12 | 0.0264 | 0.0065 | 0.007 | 0.25 | 0.049 |
| 3820721 | 0.62 | 0.0034 | 1.27 | 0.6 | 0.0064 | 0.69 | 0.5 | 0.59 | 0.61 | 5.6 | 0.0088 | 0.0056 | 0.0055 | 0.15 | 0.028 |
| 3820752 | 0.61 | 0.007 | 1.3 | 0.59 | 0.0095 | 0.56 | 0.62 | 0.61 | 0.58 | 4.68 | 0.0118 | 0.0133 | 0.0064 | 0.11 | 0.014 |
| 3822055 | 0.62 | 0.0039 | 1.38 | 0.61 | 0.0028 | 0.57 | 0.64 | 0.62 | 0.59 | 5.92 | 0.0011 | 0.0019 | 0.0037 | 0.01 | 0.01 |
| 3825428 | 0.62 | 0.0049 | 1.39 | 0.58 | 0.0197 | 0.59 | 0.57 | 0.59 | 0.58 | 5.49 | 0.014 | 0.0069 | 0.0048 | 0.05 | 0.005 |
| 3829039 | 0.63 | 0.0021 | 1.35 | 0.6 | 0.0064 | 0.56 | 0.63 | 0.61 | 0.58 | 4.81 | 0.0068 | 0.0039 | 0.0028 | 0.06 | 0.007 |
| 3829810 | 0.64 | 0.0004 | 1.56 | 0.62 | 0.0011 | 0.65 | 0.58 | 0.62 | 0.62 | 5.69 | 0.0008 | 0.0007 | 0.0005 | 0.01 | 0 |
| 3831128 | 0.61 | 0.0067 | 1.65 | 0.62 | 0.0011 | 0.61 | 0.62 | 0.63 | 0.61 | 4.89 | 0.0009 | 0.0049 | 0.0041 | 0.04 | 0.006 |
| 3831280 | 0.62 | 0.0035 | 1.26 | 0.61 | 0.0018 | 0.6 | 0.62 | 0.62 | 0.6 | 7.53 | 0.0019 | 0.0036 | 0.0027 | 0.06 | 0.007 |
| 3835412 | 0.64 | 0.0005 | 1.59 | 0.61 | 0.0028 | 0.52 | 0.69 | 0.64 | 0.59 | 3.94 | 0.0027 | 0.0042 | 0.0035 | 0.08 | 0.016 |
| 3837493 | 0.64 | 0.0009 | 1.32 | 0.59 | 0.0138 | 0.54 | 0.63 | 0.6 | 0.57 | 5.56 | 0.0188 | 0.0012 | 0.001 | 0.12 | 0.024 |
| 3838612 | 0.61 | 0.0087 | 1.27 | 0.62 | 0.0007 | 0.54 | 0.7 | 0.65 | 0.6 | 5.73 | 0.001 | 0.008 | 0.0069 | 0.26 | 0.037 |
| 3838790 | 0.63 | 0.0014 | 1.28 | 0.61 | 0.0018 | 0.53 | 0.69 | 0.64 | 0.59 | 4.65 | 0.0008 | 0.0027 | 0.0021 | 0.04 | 0.004 |
| 3840344 | 0.65 | 0.0004 | 1.25 | 0.6 | 0.0042 | 0.55 | 0.65 | 0.62 | 0.59 | 5.88 | 0.0055 | 0.0004 | 0.0004 | 0.14 | 0.021 |
| 3841271 | 0.63 | 0.0012 | 1.32 | 0.62 | 0.0007 | 0.54 | 0.7 | 0.65 | 0.6 | 4.78 | 0.0003 | 0.0027 | 0.0029 | 0.07 | 0.022 |
| 3843668 | 0.66 | 0.0001 | 1.36 | 0.64 | 0.0001 | 0.54 | 0.74 | 0.69 | 0.61 | 4.86 | 0 | 0.0002 | 0.0002 | 0.05 | 0.006 |
| 3845710 | 0.63 | 0.0018 | 1.29 | 0.61 | 0.0028 | 0.73 | 0.48 | 0.59 | 0.64 | 6.23 | 0.0038 | 0.0025 | 0.0014 | 0.07 | 0.006 |
| 3846115 | 0.62 | 0.0043 | 1.3 | 0.62 | 0.0011 | 0.57 | 0.66 | 0.64 | 0.6 | 5.13 | 0.0009 | 0.0033 | 0.0039 | 0.09 | 0.032 |
| 3846291 | 0.61 | 0.0063 | 1.26 | 0.59 | 0.0138 | 0.55 | 0.62 | 0.6 | 0.58 | 4.67 | 0.0263 | 0.015 | 0.0083 | 0.16 | 0.055 |
| 3846561 | 0.63 | 0.0015 | 1.26 | 0.61 | 0.0028 | 0.67 | 0.54 | 0.6 | 0.62 | 7.21 | 0.0022 | 0.0027 | 0.0028 | 0.15 | 0.012 |
| 3846784 | 0.62 | 0.0043 | 1.4 | 0.61 | 0.0018 | 0.65 | 0.57 | 0.61 | 0.62 | 6.04 | 0.0023 | 0.0028 | 0.0026 | 0.04 | 0.006 |
| 3847357 | 0.62 | 0.0047 | 1.32 | 0.62 | 0.0007 | 0.71 | 0.53 | 0.61 | 0.64 | 6.54 | 0.0008 | 0.0051 | 0.0039 | 0.03 | 0.003 |
| 3849774 | 0.61 | 0.0106 | 1.27 | 0.59 | 0.0095 | 0.56 | 0.62 | 0.61 | 0.58 | 5.84 | 0.0138 | 0.0185 | 0.0112 | 0.29 | 0.051 |
| 3849776 | 0.63 | 0.0019 | 1.26 | 0.62 | 0.0011 | 0.5 | 0.73 | 0.66 | 0.59 | 5.51 | 0.0006 | 0.0016 | 0.0018 | 0.2 | 0.02 |
| 3850502 | 0.64 | 0.0009 | 1.37 | 0.62 | 0.0007 | 0.64 | 0.6 | 0.63 | 0.62 | 5.57 | 0.0006 | 0.0013 | 0.0009 | 0.02 | 0.001 |
| 3851604 | 0.6 | 0.0138 | 1.27 | 0.63 | 0.0004 | 0.69 | 0.56 | 0.62 | 0.64 | 6.74 | 0.0003 | 0.0134 | 0.0108 | 0.2 | 0.017 |
| 3851902 | 0.62 | 0.004 | 1.44 | 0.61 | 0.0028 | 0.57 | 0.64 | 0.62 | 0.59 | 5.47 | 0.0015 | 0.0018 | 0.0044 | 0.03 | 0.02 |
| 3854371 | 0.63 | 0.0019 | 1.48 | 0.62 | 0.0007 | 0.52 | 0.72 | 0.66 | 0.6 | 3.6 | 0.0002 | 0.004 | 0.0045 | 0.02 | 0.012 |
| 3855101 | 0.61 | 0.0056 | 1.27 | 0.6 | 0.0064 | 0.57 | 0.62 | 0.61 | 0.59 | 6.36 | 0.0085 | 0.0061 | 0.0059 | 0.04 | 0.006 |
| 3856046 | 0.62 | 0.0035 | 1.27 | 0.57 | 0.038 | 0.45 | 0.7 | 0.61 | 0.55 | 4.39 | 0.0496 | 0.0083 | 0.0058 | 0.4 | 0.053 |
| 3857120 | 0.62 | 0.0028 | 1.3 | 0.61 | 0.0028 | 0.5 | 0.71 | 0.65 | 0.58 | 4.45 | 0.0015 | 0.0019 | 0.0024 | 0.19 | 0.066 |
| 3857884 | 0.67 | 0 | 1.3 | 0.65 | 0 | 0.57 | 0.73 | 0.69 | 0.63 | 5.25 | 0 | 0.0001 | 0.0001 | 0.02 | 0.001 |
| 3862168 | 0.63 | 0.0019 | 1.29 | 0.61 | 0.0018 | 0.67 | 0.55 | 0.61 | 0.62 | 8.4 | 0.0033 | 0.0022 | 0.0013 | 0.05 | 0.004 |
| 3862473 | 0.63 | 0.0013 | 1.27 | 0.59 | 0.0095 | 0.6 | 0.58 | 0.6 | 0.59 | 6.39 | 0.0079 | 0.0022 | 0.001 | 0.08 | 0.006 |
| 3867099 | 0.6 | 0.0112 | 1.27 | 0.62 | 0.0011 | 0.56 | 0.67 | 0.64 | 0.6 | 5.68 | 0.0015 | 0.0098 | 0.008 | 0.36 | 0.097 |
| 3869659 | 0.62 | 0.0045 | 1.27 | 0.57 | 0.038 | 0.51 | 0.63 | 0.59 | 0.56 | 4.47 | 0.0826 | 0.0092 | 0.0061 | 0.36 | 0.128 |
| 3869971 | 0.64 | 0.0009 | 1.37 | 0.59 | 0.0095 | 0.47 | 0.72 | 0.64 | 0.57 | 5.08 | 0.0112 | 0.0011 | 0.0009 | 0.09 | 0.004 |
| 3870593 | 0.64 | 0.0005 | 1.27 | 0.64 | 0.0001 | 0.63 | 0.65 | 0.65 | 0.63 | 6.4 | 0.0002 | 0.0008 | 0.0004 | 0.02 | 0.001 |
| 3872197 | 0.62 | 0.0024 | 1.73 | 0.62 | 0.0007 | 0.58 | 0.66 | 0.64 | 0.61 | 5.31 | 0.0008 | 0.0029 | 0.0018 | 0.22 | 0.015 |
| 3872208 | 0.62 | 0.0049 | 1.26 | 0.6 | 0.0042 | 0.57 | 0.63 | 0.62 | 0.59 | 5.65 | 0.0066 | 0.006 | 0.0044 | 0.19 | 0.024 |
| 3874114 | 0.65 | 0.0003 | 1.29 | 0.62 | 0.0007 | 0.55 | 0.69 | 0.65 | 0.6 | 3.09 | 0.0008 | 0.0022 | 0.0016 | 0.12 | 0.015 |
| 3874280 | 0.63 | 0.002 | 1.3 | 0.62 | 0.0007 | 0.7 | 0.54 | 0.61 | 0.64 | 7.41 | 0.0005 | 0.0036 | 0.0027 | 0.05 | 0.003 |
| 3875278 | 0.6 | 0.0148 | 1.34 | 0.57 | 0.038 | 0.53 | 0.61 | 0.59 | 0.56 | 4.97 | 0.0509 | 0.0203 | 0.0126 | 0.64 | 0.09 |
| 3878051 | 0.61 | 0.0066 | 1.45 | 0.59 | 0.0095 | 0.57 | 0.61 | 0.6 | 0.58 | 4.6 | 0.0096 | 0.0081 | 0.0047 | 0.13 | 0.027 |
| 3878053 | 0.65 | 0.0003 | 1.37 | 0.64 | 0.0001 | 0.64 | 0.64 | 0.65 | 0.64 | 7.19 | 0.0001 | 0.0004 | 0.0003 | 0.03 | 0.003 |
| 3879490 | 0.62 | 0.0037 | 1.27 | 0.54 | 0.1784 | 0.45 | 0.64 | 0.56 | 0.53 | 3.33 | 0.2201 | 0.0103 | 0.0098 | 0.27 | 0.09 |
| 3881731 | 0.64 | 0.0009 | 1.56 | 0.6 | 0.0064 | 0.63 | 0.56 | 0.6 | 0.6 | 4.8 | 0.0063 | 0.0015 | 0.001 | 0.02 | 0.003 |
| 3881914 | 0.63 | 0.0016 | 1.27 | 0.61 | 0.0028 | 0.54 | 0.67 | 0.63 | 0.59 | 6.52 | 0.0015 | 0.0014 | 0.0027 | 0.05 | 0.019 |
| 3882710 | 0.62 | 0.0037 | 1.4 | 0.61 | 0.0018 | 0.64 | 0.58 | 0.61 | 0.61 | 6.49 | 0.0015 | 0.0028 | 0.0028 | 0.07 | 0.025 |
| 3882867 | 0.64 | 0.0009 | 1.46 | 0.62 | 0.0007 | 0.57 | 0.67 | 0.64 | 0.61 | 5.07 | 0.0005 | 0.0014 | 0.0016 | 0.09 | 0.037 |
| 3883424 | 0.68 | 0 | 1.3 | 0.64 | 0.0001 | 0.62 | 0.65 | 0.65 | 0.63 | 7.1 | 0.0001 | 0 | 0 | 0.01 | 0.001 |
| 3883533 | 0.64 | 0.0009 | 1.26 | 0.65 | 0 | 0.6 | 0.7 | 0.68 | 0.63 | 6.41 | 0 | 0.0011 | 0.0009 | 0.06 | 0.005 |
| 3883537 | 0.63 | 0.0015 | 1.31 | 0.62 | 0.0007 | 0.55 | 0.69 | 0.65 | 0.6 | 6.1 | 0.0003 | 0.0016 | 0.0015 | 0.1 | 0.012 |
| 3883987 | 0.65 | 0.0003 | 1.28 | 0.63 | 0.0004 | 0.62 | 0.63 | 0.64 | 0.62 | 5.89 | 0.0018 | 0.0006 | 0.0003 | 0.02 | 0.001 |
| 3884147 | 0.62 | 0.0026 | 1.34 | 0.6 | 0.0042 | 0.57 | 0.63 | 0.62 | 0.59 | 5.86 | 0.0055 | 0.0031 | 0.0032 | 0.06 | 0.006 |
| 3884334 | 0.65 | 0.0004 | 1.27 | 0.63 | 0.0002 | 0.57 | 0.69 | 0.66 | 0.61 | 3.63 | 0.0001 | 0.0033 | 0.0013 | 0.01 | 0.001 |
| 3884673 | 0.63 | 0.0017 | 1.31 | 0.62 | 0.0011 | 0.51 | 0.72 | 0.66 | 0.59 | 3.9 | 0.0002 | 0.0006 | 0.0019 | 0.04 | 0.009 |
| 3884709 | 0.65 | 0.0002 | 1.28 | 0.62 | 0.0007 | 0.53 | 0.71 | 0.66 | 0.6 | 5.2 | 0.0005 | 0.0003 | 0.0002 | 0.18 | 0.014 |
| 3885711 | 0.62 | 0.0035 | 1.27 | 0.62 | 0.0011 | 0.6 | 0.63 | 0.63 | 0.61 | 6.49 | 0.0008 | 0.0035 | 0.0045 | 0.02 | 0.004 |
| 3886656 | 0.61 | 0.006 | 1.33 | 0.61 | 0.0018 | 0.5 | 0.72 | 0.65 | 0.59 | 5.12 | 0.0015 | 0.0096 | 0.0062 | 0.42 | 0.045 |
| 3886907 | 0.62 | 0.0037 | 1.27 | 0.63 | 0.0002 | 0.54 | 0.72 | 0.67 | 0.61 | 6.13 | 0.0001 | 0.004 | 0.0055 | 0.08 | 0.011 |
| 3887661 | 0.61 | 0.0054 | 1.25 | 0.59 | 0.0095 | 0.46 | 0.73 | 0.64 | 0.57 | 3.99 | 0.0092 | 0.0063 | 0.0049 | 0.35 | 0.057 |
| 3888081 | 0.64 | 0.0007 | 1.28 | 0.61 | 0.0018 | 0.51 | 0.71 | 0.65 | 0.59 | 5.18 | 0.0008 | 0.0003 | 0.001 | 0.05 | 0.023 |
| 3888182 | 0.61 | 0.0071 | 1.35 | 0.62 | 0.0011 | 0.71 | 0.52 | 0.61 | 0.64 | 6.72 | 0.0013 | 0.0087 | 0.0045 | 0.12 | 0.009 |
| 3888253 | 0.64 | 0.0004 | 1.35 | 0.62 | 0.0011 | 0.56 | 0.67 | 0.64 | 0.6 | 6.09 | 0.0006 | 0.0006 | 0.0004 | 0.01 | 0.001 |
| 3888289 | 0.63 | 0.0019 | 1.45 | 0.61 | 0.0018 | 0.64 | 0.58 | 0.61 | 0.61 | 5.85 | 0.003 | 0.0014 | 0.0015 | 0.05 | 0.005 |
| 3888494 | 0.6 | 0.0106 | 1.27 | 0.59 | 0.0095 | 0.56 | 0.62 | 0.61 | 0.58 | 6.07 | 0.0145 | 0.0143 | 0.0089 | 0.09 | 0.012 |
| 3888936 | 0.63 | 0.0013 | 1.3 | 0.59 | 0.0138 | 0.54 | 0.63 | 0.6 | 0.57 | 5.44 | 0.0348 | 0.0048 | 0.0019 | 0.13 | 0.016 |
| 3889140 | 0.66 | 0.0001 | 1.25 | 0.64 | 0.0001 | 0.55 | 0.72 | 0.67 | 0.61 | 6.4 | 0 | 0 | 0.0001 | 0.03 | 0.012 |
| 3889782 | 0.62 | 0.0043 | 1.34 | 0.59 | 0.0138 | 0.46 | 0.72 | 0.63 | 0.56 | 4 | 0.0055 | 0.0063 | 0.0073 | 0.03 | 0.012 |
| 3891221 | 0.6 | 0.0125 | 1.36 | 0.62 | 0.0011 | 0.6 | 0.63 | 0.63 | 0.61 | 4.76 | 0.0015 | 0.0088 | 0.0108 | 0.2 | 0.03 |
| 3891257 | 0.66 | 0.0002 | 1.28 | 0.61 | 0.0018 | 0.6 | 0.62 | 0.62 | 0.6 | 7.82 | 0.003 | 0.0003 | 0.0003 | 0.03 | 0.001 |
| 3892672 | 0.64 | 0.0007 | 1.28 | 0.64 | 0.0001 | 0.6 | 0.67 | 0.66 | 0.62 | 8.08 | 0.0001 | 0.0005 | 0.0009 | 0.09 | 0.012 |
| 3894074 | 0.61 | 0.0066 | 1.25 | 0.58 | 0.0276 | 0.5 | 0.66 | 0.6 | 0.56 | 3.6 | 0.0363 | 0.0046 | 0.0073 | 0.08 | 0.01 |
| 3894508 | 0.63 | 0.0013 | 1.49 | 0.62 | 0.0011 | 0.59 | 0.64 | 0.63 | 0.61 | 5.67 | 0.0011 | 0.0014 | 0.0008 | 0.03 | 0.001 |
| 3894602 | 0.61 | 0.0089 | 1.4 | 0.6 | 0.0042 | 0.63 | 0.57 | 0.6 | 0.6 | 5.52 | 0.0046 | 0.008 | 0.0058 | 0.09 | 0.012 |
| 3895000 | 0.61 | 0.0069 | 1.31 | 0.63 | 0.0004 | 0.65 | 0.6 | 0.63 | 0.63 | 6.22 | 0.0008 | 0.0065 | 0.0048 | 0.06 | 0.012 |
| 3896202 | 0.6 | 0.0144 | 1.39 | 0.57 | 0.038 | 0.52 | 0.62 | 0.59 | 0.56 | 5.28 | 0.04 | 0.0192 | 0.0162 | 0.08 | 0.019 |
| 3898306 | 0.6 | 0.0122 | 1.29 | 0.58 | 0.0276 | 0.53 | 0.62 | 0.59 | 0.56 | 3.04 | 0.0448 | 0.0531 | 0.0268 | 0.49 | 0.237 |
| 3901666 | 0.6 | 0.0115 | 1.35 | 0.6 | 0.0064 | 0.65 | 0.54 | 0.59 | 0.6 | 6.28 | 0.0079 | 0.0126 | 0.0109 | 0.07 | 0.011 |
| 3902984 | 0.64 | 0.0009 | 1.34 | 0.59 | 0.0095 | 0.54 | 0.64 | 0.61 | 0.58 | 5.5 | 0.0097 | 0.0015 | 0.0015 | 0.03 | 0.014 |
| 3903290 | 0.65 | 0.0004 | 1.32 | 0.64 | 0.0001 | 0.62 | 0.65 | 0.65 | 0.63 | 6.67 | 0.0002 | 0.0004 | 0.0002 | 0.04 | 0.005 |
| 3904028 | 0.64 | 0.0004 | 1.39 | 0.54 | 0.1784 | 0.3 | 0.8 | 0.6 | 0.52 | 4.37 | 0.1663 | 0.0014 | 0.0027 | 0.44 | 0.225 |
| 3904029 | 0.64 | 0.0005 | 1.25 | 0.63 | 0.0004 | 0.58 | 0.67 | 0.65 | 0.61 | 8.51 | 0.0008 | 0.0002 | 0.0004 | 0.04 | 0.006 |
| 3904255 | 0.64 | 0.0006 | 1.27 | 0.63 | 0.0002 | 0.58 | 0.68 | 0.66 | 0.61 | 5.76 | 0.0004 | 0.0015 | 0.0011 | 0.08 | 0.013 |
| 3904257 | 0.64 | 0.0005 | 1.34 | 0.64 | 0.0001 | 0.69 | 0.59 | 0.64 | 0.65 | 6.17 | 0.0001 | 0.0007 | 0.0005 | 0.1 | 0.014 |
| 3904276 | 0.64 | 0.0005 | 1.28 | 0.61 | 0.0018 | 0.57 | 0.65 | 0.63 | 0.6 | 4.36 | 0.0014 | 0.0012 | 0.0019 | 0.03 | 0.023 |
| 3904281 | 0.61 | 0.0081 | 1.27 | 0.61 | 0.0028 | 0.47 | 0.76 | 0.66 | 0.58 | 5.04 | 0.0004 | 0.0023 | 0.0035 | 0.19 | 0.034 |
| 3906196 | 0.61 | 0.008 | 1.31 | 0.59 | 0.0095 | 0.58 | 0.6 | 0.6 | 0.58 | 4.26 | 0.0182 | 0.009 | 0.0083 | 0.61 | 0.127 |
| 3907788 | 0.61 | 0.007 | 1.45 | 0.62 | 0.0007 | 0.62 | 0.62 | 0.63 | 0.62 | 5.04 | 0.0003 | 0.0049 | 0.005 | 0.04 | 0.012 |
| 3908789 | 0.62 | 0.0024 | 1.34 | 0.63 | 0.0002 | 0.62 | 0.64 | 0.64 | 0.62 | 7.29 | 0.0004 | 0.0015 | 0.001 | 0.14 | 0.009 |
| 3909376 | 0.61 | 0.0077 | 1.25 | 0.58 | 0.0276 | 0.5 | 0.66 | 0.6 | 0.56 | 3.83 | 0.0184 | 0.0035 | 0.0033 | 0.3 | 0.032 |
| 3911474 | 0.64 | 0.0007 | 1.36 | 0.62 | 0.0007 | 0.62 | 0.62 | 0.63 | 0.62 | 6.7 | 0.0013 | 0.0011 | 0.0005 | 0.07 | 0.003 |
| 3911564 | 0.65 | 0.0002 | 1.37 | 0.67 | 0 | 0.63 | 0.7 | 0.69 | 0.65 | 6.02 | 0 | 0.0002 | 0.0002 | 0.04 | 0.003 |
| 3911706 | 0.64 | 0.0006 | 1.31 | 0.63 | 0.0002 | 0.61 | 0.65 | 0.65 | 0.62 | 5.56 | 0.0004 | 0.0008 | 0.0007 | 0.03 | 0.002 |
| 3911738 | 0.66 | 0.0001 | 1.3 | 0.63 | 0.0004 | 0.65 | 0.6 | 0.63 | 0.63 | 7.3 | 0.0015 | 0.0003 | 0.0002 | 0.02 | 0.001 |
| 3911801 | 0.63 | 0.0016 | 1.44 | 0.63 | 0.0002 | 0.65 | 0.61 | 0.63 | 0.63 | 5.91 | 0.0003 | 0.0007 | 0.0007 | 0.02 | 0.002 |
| 3911815 | 0.62 | 0.004 | 1.48 | 0.61 | 0.0018 | 0.55 | 0.67 | 0.64 | 0.59 | 5.48 | 0.003 | 0.003 | 0.0028 | 0.13 | 0.019 |
| 3913564 | 0.62 | 0.0029 | 1.31 | 0.57 | 0.038 | 0.55 | 0.59 | 0.58 | 0.56 | 4.58 | 0.0329 | 0.0065 | 0.004 | 0.19 | 0.068 |
| 3915125 | 0.64 | 0.0006 | 1.35 | 0.57 | 0.0513 | 0.38 | 0.77 | 0.62 | 0.54 | 4.05 | 0.0575 | 0.0031 | 0.0023 | 0.03 | 0.004 |
| 3917896 | 0.65 | 0.0003 | 1.38 | 0.62 | 0.0007 | 0.6 | 0.64 | 0.64 | 0.61 | 5.35 | 0.0006 | 0.0012 | 0.0007 | 0.03 | 0.003 |
| 3918586 | 0.62 | 0.0031 | 1.37 | 0.61 | 0.0028 | 0.45 | 0.78 | 0.67 | 0.58 | 4.15 | 0.0018 | 0.0015 | 0.0016 | 0.09 | 0.017 |
| 3918651 | 0.61 | 0.0095 | 1.27 | 0.59 | 0.0138 | 0.54 | 0.63 | 0.6 | 0.57 | 4.24 | 0.0113 | 0.0074 | 0.0075 | 0.13 | 0.032 |
| 3918723 | 0.64 | 0.0007 | 1.32 | 0.64 | 0.0001 | 0.61 | 0.67 | 0.66 | 0.63 | 6.09 | 0.0001 | 0.0007 | 0.0011 | 0.09 | 0.012 |
| 3918734 | 0.63 | 0.0016 | 1.31 | 0.62 | 0.0011 | 0.56 | 0.67 | 0.64 | 0.6 | 4.43 | 0.0003 | 0.0011 | 0.0021 | 0.28 | 0.102 |
| 3919024 | 0.63 | 0.0012 | 1.4 | 0.65 | 0 | 0.76 | 0.54 | 0.63 | 0.69 | 6.04 | 0.0001 | 0.0007 | 0.0004 | 0.02 | 0.001 |
| 3920401 | 0.63 | 0.0012 | 1.38 | 0.6 | 0.0042 | 0.5 | 0.71 | 0.64 | 0.58 | 4.26 | 0.0031 | 0.0037 | 0.0031 | 0.25 | 0.033 |
| 3920456 | 0.64 | 0.0008 | 1.26 | 0.62 | 0.0011 | 0.63 | 0.6 | 0.62 | 0.61 | 6.62 | 0.0009 | 0.001 | 0.0009 | 0.15 | 0.01 |
| 3920487 | 0.66 | 0.0001 | 1.28 | 0.61 | 0.0028 | 0.54 | 0.67 | 0.63 | 0.59 | 5.06 | 0.0025 | 0.0002 | 0.0003 | 0.07 | 0.015 |
| 3922023 | 0.61 | 0.0053 | 1.29 | 0.55 | 0.114 | 0.32 | 0.8 | 0.62 | 0.53 | 4.57 | 0.0529 | 0.0052 | 0.011 | 0.06 | 0.059 |
| 3922993 | 0.63 | 0.0021 | 1.36 | 0.61 | 0.0018 | 0.59 | 0.63 | 0.63 | 0.6 | 4.28 | 0.0014 | 0.0026 | 0.0057 | 0.22 | 0.128 |
| 3924758 | 0.61 | 0.008 | 1.27 | 0.55 | 0.1438 | 0.4 | 0.7 | 0.58 | 0.53 | 4.56 | 0.2256 | 0.0594 | 0.0262 | 0.99 | 0.182 |
| 3925641 | 0.66 | 0.0001 | 1.29 | 0.59 | 0.0138 | 0.43 | 0.76 | 0.64 | 0.56 | 5.51 | 0.0025 | 0.0009 | 0.0009 | 0.15 | 0.072 |
| 3926090 | 0.63 | 0.001 | 1.28 | 0.62 | 0.0011 | 0.48 | 0.77 | 0.68 | 0.59 | 4.4 | 0.0002 | 0.0008 | 0.001 | 0.15 | 0.027 |
| 3927180 | 0.6 | 0.0122 | 1.49 | 0.59 | 0.0095 | 0.54 | 0.64 | 0.61 | 0.58 | 4.11 | 0.0143 | 0.016 | 0.0108 | 0.29 | 0.049 |
| 3927228 | 0.62 | 0.0029 | 1.26 | 0.6 | 0.0042 | 0.76 | 0.44 | 0.58 | 0.64 | 6.24 | 0.0048 | 0.0042 | 0.0027 | 0.06 | 0.008 |
| 3927229 | 0.64 | 0.0009 | 1.42 | 0.62 | 0.0011 | 0.66 | 0.57 | 0.61 | 0.62 | 5.6 | 0.0025 | 0.0015 | 0.0009 | 0.06 | 0.009 |
| 3928670 | 0.63 | 0.0011 | 1.31 | 0.6 | 0.0064 | 0.59 | 0.6 | 0.61 | 0.59 | 5.2 | 0.008 | 0.0011 | 0.0015 | 0.07 | 0.022 |
| 3929943 | 0.63 | 0.0018 | 1.36 | 0.59 | 0.0095 | 0.59 | 0.59 | 0.6 | 0.59 | 6.67 | 0.008 | 0.0008 | 0.0013 | 0.1 | 0.018 |
| 3934699 | 0.6 | 0.0124 | 1.4 | 0.61 | 0.0028 | 0.6 | 0.61 | 0.62 | 0.6 | 4.71 | 0.0037 | 0.0141 | 0.0112 | 0.34 | 0.081 |
| 3935246 | 0.61 | 0.0089 | 1.41 | 0.63 | 0.0004 | 0.63 | 0.62 | 0.63 | 0.62 | 4.97 | 0.0002 | 0.0101 | 0.0106 | 0.04 | 0.024 |
| 3935301 | 0.61 | 0.007 | 1.26 | 0.63 | 0.0004 | 0.65 | 0.6 | 0.63 | 0.63 | 7.03 | 0.0005 | 0.009 | 0.0075 | 0.12 | 0.014 |
| 3936897 | 0.62 | 0.0029 | 1.36 | 0.6 | 0.0064 | 0.51 | 0.68 | 0.63 | 0.58 | 4.69 | 0.0095 | 0.0085 | 0.0039 | 0.31 | 0.019 |
| 3939407 | 0.61 | 0.0069 | 1.27 | 0.63 | 0.0004 | 0.69 | 0.56 | 0.62 | 0.64 | 6.85 | 0.0008 | 0.0078 | 0.0044 | 0.09 | 0.005 |
| 3942668 | 0.62 | 0.0029 | 1.27 | 0.62 | 0.0011 | 0.62 | 0.61 | 0.62 | 0.61 | 7.02 | 0.0021 | 0.0038 | 0.0022 | 0.13 | 0.005 |
| 3945331 | 0.62 | 0.0026 | 1.42 | 0.62 | 0.0011 | 0.67 | 0.56 | 0.61 | 0.63 | 4.9 | 0.0018 | 0.0031 | 0.0019 | 0.08 | 0.008 |
| 3945370 | 0.63 | 0.0017 | 1.38 | 0.61 | 0.0018 | 0.5 | 0.72 | 0.65 | 0.59 | 5.23 | 0.0007 | 0.0038 | 0.0023 | 0.16 | 0.036 |
| 3946374 | 0.61 | 0.008 | 1.27 | 0.59 | 0.0138 | 0.44 | 0.74 | 0.64 | 0.56 | 4.93 | 0.0071 | 0.0068 | 0.0074 | 0.03 | 0.004 |
| 3946589 | 0.6 | 0.0116 | 1.29 | 0.57 | 0.038 | 0.51 | 0.63 | 0.59 | 0.56 | 5.2 | 0.0496 | 0.0169 | 0.0147 | 0.18 | 0.084 |
| 3946677 | 0.61 | 0.0071 | 1.31 | 0.58 | 0.0197 | 0.5 | 0.67 | 0.61 | 0.56 | 4.18 | 0.0159 | 0.0107 | 0.0124 | 0.24 | 0.132 |
| 3953804 | 0.63 | 0.0019 | 1.28 | 0.62 | 0.0011 | 0.65 | 0.58 | 0.62 | 0.62 | 4.64 | 0.0018 | 0.0023 | 0.0028 | 0.08 | 0.013 |
| 3956591 | 0.62 | 0.0049 | 1.28 | 0.61 | 0.0028 | 0.66 | 0.55 | 0.6 | 0.61 | 7.26 | 0.0092 | 0.0106 | 0.0034 | 0.14 | 0.019 |
| 3957262 | 0.63 | 0.0011 | 1.47 | 0.61 | 0.0028 | 0.51 | 0.7 | 0.64 | 0.58 | 5.05 | 0.0034 | 0.0026 | 0.0015 | 0.09 | 0.016 |
| 3959259 | 0.63 | 0.0015 | 1.25 | 0.61 | 0.0018 | 0.62 | 0.6 | 0.62 | 0.61 | 5.32 | 0.0004 | 0.0005 | 0.002 | 0.06 | 0.023 |
| 3959453 | 0.62 | 0.004 | 1.34 | 0.59 | 0.0095 | 0.61 | 0.57 | 0.6 | 0.59 | 6.98 | 0.0147 | 0.0054 | 0.0038 | 0.08 | 0.008 |
| 3960634 | 0.66 | 0.0001 | 1.52 | 0.62 | 0.0007 | 0.64 | 0.6 | 0.63 | 0.62 | 5.96 | 0.0006 | 0.0002 | 0.0002 | 0.03 | 0.002 |
| 3961482 | 0.62 | 0.004 | 1.26 | 0.6 | 0.0064 | 0.6 | 0.59 | 0.6 | 0.59 | 5.66 | 0.0059 | 0.0037 | 0.0029 | 0.11 | 0.01 |
| 3962268 | 0.64 | 0.0007 | 1.65 | 0.62 | 0.0007 | 0.66 | 0.58 | 0.62 | 0.63 | 5.98 | 0.0009 | 0.0004 | 0.0004 | 0.02 | 0.002 |
| 3962596 | 0.65 | 0.0002 | 1.42 | 0.61 | 0.0028 | 0.6 | 0.61 | 0.62 | 0.6 | 5.63 | 0.0024 | 0.0002 | 0.0002 | 0.07 | 0.005 |
| 3969510 | 0.62 | 0.0041 | 1.32 | 0.62 | 0.0011 | 0.48 | 0.77 | 0.68 | 0.59 | 4.12 | 0.0003 | 0.0049 | 0.0061 | 0.13 | 0.05 |
| 3969511 | 0.64 | 0.0008 | 1.65 | 0.64 | 0.0001 | 0.64 | 0.64 | 0.65 | 0.64 | 5.23 | 0.0001 | 0.0003 | 0.0003 | 0.02 | 0.004 |
| 3970894 | 0.66 | 0.0001 | 1.32 | 0.63 | 0.0004 | 0.64 | 0.61 | 0.63 | 0.63 | 7.89 | 0.0006 | 0.0002 | 0.0002 | 0.01 | 0 |
| 3974757 | 0.61 | 0.0063 | 1.29 | 0.55 | 0.114 | 0.5 | 0.6 | 0.57 | 0.54 | 4.69 | 0.079 | 0.0048 | 0.0121 | 0.04 | 0.052 |
| 3974789 | 0.63 | 0.0017 | 1.43 | 0.62 | 0.0007 | 0.59 | 0.65 | 0.64 | 0.61 | 5.33 | 0.0008 | 0.0017 | 0.0013 | 0.09 | 0.01 |
| 3981154 | 0.63 | 0.0014 | 1.42 | 0.62 | 0.0011 | 0.55 | 0.68 | 0.64 | 0.6 | 4.28 | 0.0011 | 0.0013 | 0.0015 | 0.25 | 0.027 |
| 3981752 | 0.62 | 0.0038 | 1.32 | 0.58 | 0.0276 | 0.51 | 0.64 | 0.6 | 0.56 | 5.23 | 0.0256 | 0.0037 | 0.0035 | 0.84 | 0.17 |
| 3981906 | 0.63 | 0.0011 | 1.34 | 0.62 | 0.0011 | 0.58 | 0.65 | 0.63 | 0.6 | 6.58 | 0.0007 | 0.0011 | 0.0012 | 0.06 | 0.008 |
| 3982421 | 0.6 | 0.012 | 1.38 | 0.62 | 0.0007 | 0.61 | 0.63 | 0.63 | 0.61 | 5.83 | 0.0013 | 0.0116 | 0.0095 | 0.15 | 0.024 |
| 3985558 | 0.63 | 0.0011 | 1.31 | 0.63 | 0.0004 | 0.64 | 0.61 | 0.63 | 0.63 | 7.57 | 0.0005 | 0.0009 | 0.0009 | 0.01 | 0.001 |
| 3985635 | 0.66 | 0.0001 | 1.51 | 0.61 | 0.0018 | 0.58 | 0.64 | 0.63 | 0.6 | 4.97 | 0.0019 | 0.0006 | 0.0003 | 0.1 | 0.004 |
| 3986857 | 0.62 | 0.0031 | 1.31 | 0.57 | 0.038 | 0.46 | 0.69 | 0.61 | 0.55 | 5.54 | 0.0584 | 0.0041 | 0.0035 | 0.06 | 0.003 |
| 3987532 | 0.6 | 0.0126 | 1.45 | 0.57 | 0.038 | 0.52 | 0.62 | 0.59 | 0.56 | 4.8 | 0.0561 | 0.0214 | 0.0129 | 0.31 | 0.051 |
| 3988214 | 0.61 | 0.0053 | 1.32 | 0.62 | 0.0011 | 0.56 | 0.67 | 0.64 | 0.6 | 5.55 | 0.001 | 0.0153 | 0.0097 | 0.16 | 0.027 |
| 3989205 | 0.63 | 0.0019 | 1.27 | 0.61 | 0.0018 | 0.67 | 0.55 | 0.61 | 0.62 | 6.95 | 0.0029 | 0.0012 | 0.0009 | 0.01 | 0.002 |
| 3989770 | 0.62 | 0.0048 | 1.29 | 0.6 | 0.0064 | 0.46 | 0.74 | 0.65 | 0.57 | 3.76 | 0.0068 | 0.0071 | 0.0049 | 0.27 | 0.017 |
| 3991722 | 0.61 | 0.0083 | 1.44 | 0.61 | 0.0028 | 0.51 | 0.7 | 0.64 | 0.58 | 4.63 | 0.002 | 0.0173 | 0.0135 | 0.04 | 0.005 |
| 3993347 | 0.61 | 0.0081 | 1.27 | 0.59 | 0.0138 | 0.54 | 0.63 | 0.6 | 0.57 | 7.42 | 0.0204 | 0.0062 | 0.007 | 0.09 | 0.024 |
| 3993349 | 0.63 | 0.002 | 1.35 | 0.62 | 0.0007 | 0.66 | 0.58 | 0.62 | 0.63 | 7.12 | 0.0007 | 0.002 | 0.0021 | 0.05 | 0.014 |
| 4004822 | 0.63 | 0.0013 | 1.26 | 0.66 | 0 | 0.6 | 0.71 | 0.69 | 0.64 | 5.18 | 0 | 0.0026 | 0.0021 | 0.07 | 0.023 |
| 4007593 | 0.62 | 0.0049 | 1.44 | 0.59 | 0.0095 | 0.57 | 0.61 | 0.6 | 0.58 | 5.78 | 0.0124 | 0.0081 | 0.0045 | 0.16 | 0.023 |
| 4009318 | 0.62 | 0.0024 | 1.31 | 0.63 | 0.0002 | 0.71 | 0.55 | 0.62 | 0.65 | 7.98 | 0.0003 | 0.0028 | 0.002 | 0.08 | 0.008 |
| 4009451 | 0.63 | 0.0018 | 1.26 | 0.61 | 0.0018 | 0.55 | 0.67 | 0.64 | 0.59 | 4.75 | 0.0007 | 0.0031 | 0.0045 | 0.08 | 0.038 |
| 4011604 | 0.61 | 0.0051 | 1.3 | 0.6 | 0.0064 | 0.64 | 0.55 | 0.6 | 0.6 | 5.92 | 0.0062 | 0.0047 | 0.0047 | 0.08 | 0.012 |
| 4011967 | 0.62 | 0.0029 | 1.26 | 0.6 | 0.0042 | 0.55 | 0.65 | 0.62 | 0.59 | 6.66 | 0.0034 | 0.0017 | 0.0026 | 0.05 | 0.011 |
| 4012169 | 0.63 | 0.0016 | 1.29 | 0.63 | 0.0002 | 0.55 | 0.71 | 0.67 | 0.61 | 6.31 | 0.0003 | 0.0026 | 0.0022 | 0.12 | 0.008 |
| 4012627 | 0.64 | 0.0006 | 1.3 | 0.63 | 0.0002 | 0.56 | 0.7 | 0.66 | 0.61 | 6.17 | 0.0003 | 0.0009 | 0.0007 | 0.08 | 0.005 |
| 4012632 | 0.63 | 0.0022 | 1.27 | 0.61 | 0.0018 | 0.52 | 0.7 | 0.65 | 0.59 | 4.67 | 0.001 | 0.0024 | 0.0025 | 0.16 | 0.04 |
| 4012759 | 0.63 | 0.0011 | 1.46 | 0.6 | 0.0064 | 0.52 | 0.67 | 0.62 | 0.58 | 4.18 | 0.0062 | 0.0023 | 0.0022 | 0.15 | 0.017 |
| 4013274 | 0.64 | 0.0005 | 1.29 | 0.62 | 0.0011 | 0.53 | 0.7 | 0.65 | 0.59 | 4.28 | 0.001 | 0.002 | 0.0017 | 0.1 | 0.014 |
| 4013282 | 0.65 | 0.0002 | 1.43 | 0.6 | 0.0042 | 0.58 | 0.62 | 0.61 | 0.59 | 5.8 | 0.004 | 0.0002 | 0.0002 | 0.05 | 0.009 |
| 4015535 | 0.65 | 0.0003 | 1.29 | 0.64 | 0.0001 | 0.65 | 0.63 | 0.65 | 0.64 | 5.1 | 0.0001 | 0.0001 | 0.0004 | 0 | 0.001 |
| 4016549 | 0.65 | 0.0002 | 1.48 | 0.63 | 0.0002 | 0.52 | 0.74 | 0.68 | 0.6 | 5.33 | 0.0001 | 0.0003 | 0.0003 | 0.05 | 0.006 |
| 4016573 | 0.63 | 0.001 | 1.29 | 0.61 | 0.0018 | 0.6 | 0.62 | 0.62 | 0.6 | 7.57 | 0.0029 | 0.0007 | 0.0005 | 0.04 | 0.004 |
| 4019367 | 0.62 | 0.0035 | 1.25 | 0.62 | 0.0011 | 0.58 | 0.65 | 0.63 | 0.6 | 5.61 | 0.0008 | 0.0017 | 0.0027 | 0.08 | 0.02 |
| 4019418 | 0.65 | 0.0003 | 1.26 | 0.65 | 0 | 0.67 | 0.63 | 0.65 | 0.65 | 5.4 | 0.0001 | 0.003 | 0.0011 | 0.08 | 0.011 |
| 4019610 | 0.63 | 0.0016 | 1.3 | 0.62 | 0.0011 | 0.65 | 0.58 | 0.62 | 0.62 | 6.07 | 0.002 | 0.0027 | 0.0014 | 0.13 | 0.012 |
| 4020462 | 0.64 | 0.0005 | 1.26 | 0.58 | 0.0197 | 0.43 | 0.74 | 0.63 | 0.56 | 4.86 | 0.0209 | 0.0031 | 0.0015 | 0.06 | 0.002 |
| 4024375 | 0.62 | 0.0026 | 1.44 | 0.62 | 0.0011 | 0.64 | 0.59 | 0.62 | 0.62 | 5.55 | 0.0011 | 0.0022 | 0.0028 | 0.05 | 0.01 |
| 4024376 | 0.62 | 0.0026 | 1.41 | 0.62 | 0.0011 | 0.55 | 0.68 | 0.64 | 0.6 | 5.48 | 0.0007 | 0.0031 | 0.0043 | 0.06 | 0.022 |
| 4024377 | 0.61 | 0.0082 | 1.29 | 0.59 | 0.0095 | 0.61 | 0.57 | 0.6 | 0.59 | 6.83 | 0.0111 | 0.0085 | 0.0086 | 0.2 | 0.055 |
| 4024378 | 0.62 | 0.0032 | 1.53 | 0.6 | 0.0064 | 0.55 | 0.64 | 0.62 | 0.58 | 5.34 | 0.0059 | 0.0024 | 0.0038 | 0.05 | 0.036 |
| 4024379 | 0.64 | 0.0005 | 1.46 | 0.61 | 0.0018 | 0.59 | 0.63 | 0.63 | 0.6 | 6.42 | 0.0017 | 0.0004 | 0.0005 | 0.01 | 0.002 |
| 4030986 | 0.66 | 0.0001 | 1.42 | 0.63 | 0.0004 | 0.52 | 0.73 | 0.67 | 0.6 | 5.64 | 0.0002 | 0.0001 | 0.0001 | 0.03 | 0.003 |
| 4034997 | 0.64 | 0.0005 | 1.33 | 0.62 | 0.0011 | 0.55 | 0.68 | 0.64 | 0.6 | 4.38 | 0.0008 | 0.0008 | 0.0014 | 0.04 | 0.014 |
| 4035834 | 0.62 | 0.0043 | 1.28 | 0.57 | 0.038 | 0.64 | 0.5 | 0.57 | 0.58 | 6.53 | 0.0797 | 0.0152 | 0.0075 | 0.35 | 0.053 |
| 4035835 | 0.67 | 0 | 1.35 | 0.65 | 0 | 0.55 | 0.76 | 0.7 | 0.62 | 4.43 | 0 | 0 | 0.0001 | 0.04 | 0.009 |
| 4035838 | 0.63 | 0.0013 | 1.28 | 0.62 | 0.0011 | 0.53 | 0.7 | 0.65 | 0.59 | 6.9 | 0.0013 | 0.0023 | 0.0016 | 0.13 | 0.031 |
| 4035839 | 0.63 | 0.0015 | 1.27 | 0.61 | 0.0018 | 0.58 | 0.64 | 0.63 | 0.6 | 6.89 | 0.0023 | 0.0017 | 0.0012 | 0.12 | 0.039 |
| 4040526 | 0.64 | 0.0007 | 1.31 | 0.61 | 0.0018 | 0.67 | 0.55 | 0.61 | 0.62 | 7.04 | 0.0017 | 0.001 | 0.0006 | 0.01 | 0.002 |
| 4041367 | 0.61 | 0.0088 | 1.27 | 0.57 | 0.0513 | 0.51 | 0.62 | 0.58 | 0.55 | 4.39 | 0.0584 | 0.0126 | 0.0127 | 0.03 | 0.011 |
| 4041822 | 0.63 | 0.0016 | 1.31 | 0.6 | 0.0042 | 0.73 | 0.47 | 0.59 | 0.63 | 5.7 | 0.0031 | 0.0016 | 0.0018 | 0.05 | 0.01 |
| 4043125 | 0.61 | 0.0071 | 1.28 | 0.58 | 0.0276 | 0.53 | 0.62 | 0.59 | 0.56 | 4.38 | 0.0533 | 0.0202 | 0.0115 | 0.58 | 0.054 |
| 4044199 | 0.62 | 0.0039 | 1.26 | 0.62 | 0.0011 | 0.57 | 0.66 | 0.64 | 0.6 | 6.34 | 0.0007 | 0.0053 | 0.0065 | 0.2 | 0.082 |
| 4044413 | 0.62 | 0.003 | 1.28 | 0.63 | 0.0004 | 0.55 | 0.7 | 0.66 | 0.61 | 4.54 | 0.0006 | 0.0095 | 0.0041 | 0.27 | 0.054 |
| 4047577 | 0.61 | 0.0085 | 1.42 | 0.63 | 0.0004 | 0.67 | 0.58 | 0.62 | 0.63 | 6.05 | 0.0006 | 0.0089 | 0.0043 | 0.18 | 0.014 |
| 4052399 | 0.65 | 0.0002 | 1.42 | 0.65 | 0 | 0.57 | 0.72 | 0.68 | 0.62 | 5.66 | 0 | 0.0005 | 0.0004 | 0.07 | 0.006 |
| 4055295 | 0.65 | 0.0002 | 1.4 | 0.6 | 0.0042 | 0.55 | 0.65 | 0.62 | 0.59 | 6.06 | 0.0033 | 0.0003 | 0.0002 | 0.11 | 0.016 |
| 4055302 | 0.66 | 0.0001 | 1.27 | 0.61 | 0.0028 | 0.54 | 0.67 | 0.63 | 0.59 | 5.2 | 0.0022 | 0.0003 | 0.0003 | 0.01 | 0.003 |
| 2720180 | 0.58 | 0.0585 | 1.53 | 0.57 | 0.038 | 0.56 | 0.58 | 0.58 | 0.56 | 5.14 | 0.0794 | 0.1125 | 0.0625 | 0.41 | 0.046 |
| 2347642 | 0.69 | 0 | 1.12 | 0.62 | 0.0011 | 0.42 | 0.83 | 0.71 | 0.58 | 6.34 | 0.0001 | 0 | 0.0001 | 0 | 0.002 |
| 2395344 | 0.68 | 0 | 1.17 | 0.65 | 0 | 0.75 | 0.55 | 0.63 | 0.68 | 4.74 | 0 | 0 | 0 | 0.01 | 0 |
| 2397069 | 0.69 | 0 | 1.12 | 0.65 | 0 | 0.57 | 0.72 | 0.68 | 0.62 | 6.12 | 0 | 0 | 0 | 0 | 0.003 |
| 2430377 | 0.69 | 0 | 1.09 | 0.63 | 0.0004 | 0.59 | 0.66 | 0.65 | 0.61 | 5.44 | 0.0007 | 0.0001 | 0 | 0 | 0.001 |
| 2448270 | 0.68 | 0 | 1.08 | 0.63 | 0.0004 | 0.61 | 0.64 | 0.64 | 0.62 | 6.84 | 0.001 | 0.0001 | 0 | 0.01 | 0 |
| 2553581 | 0.7 | 0 | 1.15 | 0.63 | 0.0004 | 0.58 | 0.67 | 0.65 | 0.61 | 5.17 | 0.0003 | 0 | 0 | 0 | 0.001 |
| 2600700 | 0.69 | 0 | 1.14 | 0.63 | 0.0002 | 0.66 | 0.6 | 0.63 | 0.63 | 4.66 | 0.0005 | 0 | 0 | 0 | 0 |
| 2624519 | 0.68 | 0 | 1.08 | 0.62 | 0.0007 | 0.74 | 0.5 | 0.6 | 0.65 | 4.49 | 0.0014 | 0.0004 | 0.0001 | 0.02 | 0 |
| 2651846 | 0.68 | 0 | 1.17 | 0.64 | 0.0001 | 0.68 | 0.6 | 0.64 | 0.65 | 2.99 | 0.0001 | 0 | 0 | 0 | 0.002 |
| 2682441 | 0.68 | 0 | 1.14 | 0.65 | 0 | 0.57 | 0.73 | 0.69 | 0.63 | 6.35 | 0 | 0 | 0 | 0.01 | 0.001 |
| 2704512 | 0.69 | 0 | 1.11 | 0.6 | 0.0042 | 0.53 | 0.67 | 0.63 | 0.58 | 4.5 | 0.0047 | 0 | 0 | 0 | 0 |
| 2704702 | 0.68 | 0 | 1.11 | 0.65 | 0 | 0.68 | 0.61 | 0.64 | 0.65 | 7.75 | 0.0001 | 0 | 0.0001 | 0.04 | 0.004 |
| 2902832 | 0.68 | 0 | 0.97 | 0.39 | 0.9996 | 0.5 | 0.28 | 0.42 | 0.35 | 6.72 | 0.001 | 0 | 0.0001 | 0.01 | 0.001 |
| 2918558 | 0.68 | 0 | 1.17 | 0.61 | 0.0018 | 0.4 | 0.84 | 0.71 | 0.57 | 4.99 | 0.0004 | 0.0001 | 0 | 0.01 | 0 |
| 3031661 | 0.69 | 0 | 0.95 | 0.35 | 1 | 0.55 | 0.14 | 0.4 | 0.24 | 6.09 | 0 | 0.0001 | 0 | 0.01 | 0 |
| 3212761 | 0.68 | 0 | 1.18 | 0.63 | 0.0002 | 0.58 | 0.68 | 0.66 | 0.61 | 4.32 | 0.0002 | 0 | 0.0001 | 0 | 0 |
| 3268183 | 0.68 | 0 | 1.04 | 0.63 | 0.0004 | 0.46 | 0.81 | 0.71 | 0.59 | 6.98 | 0.0001 | 0 | 0 | 0 | 0.001 |
| 3472753 | 0.69 | 0 | 0.96 | 0.37 | 1 | 0.42 | 0.32 | 0.39 | 0.34 | 7.01 | 0.0006 | 0.0001 | 0.0001 | 0 | 0 |
| 3501557 | 0.68 | 0 | 1.23 | 0.64 | 0.0001 | 0.58 | 0.69 | 0.66 | 0.62 | 4.53 | 0.0003 | 0.0001 | 0 | 0.01 | 0 |
| 3543621 | 0.69 | 0 | 0.91 | 0.37 | 1 | 0.51 | 0.21 | 0.4 | 0.3 | 4.9 | 0.0001 | 0.0001 | 0 | 0.04 | 0.002 |
| 3551833 | 0.69 | 0 | 1.13 | 0.65 | 0 | 0.58 | 0.72 | 0.69 | 0.63 | 6.09 | 0 | 0 | 0 | 0 | 0 |
| 3562041 | 0.69 | 0 | 1.24 | 0.64 | 0.0001 | 0.57 | 0.71 | 0.67 | 0.62 | 6.37 | 0 | 0 | 0 | 0 | 0 |
| 3661099 | 0.69 | 0 | 1.09 | 0.63 | 0.0004 | 0.51 | 0.74 | 0.68 | 0.6 | 6.7 | 0.0003 | 0 | 0.0001 | 0.1 | 0.013 |
| 3667907 | 0.69 | 0 | 1.05 | 0.64 | 0.0001 | 0.56 | 0.72 | 0.68 | 0.62 | 8.01 | 0 | 0 | 0 | 0 | 0 |
| 3701840 | 0.68 | 0 | 1.09 | 0.61 | 0.0018 | 0.49 | 0.74 | 0.66 | 0.58 | 5.5 | 0.0009 | 0 | 0 | 0 | 0.001 |
| 3840609 | 0.69 | 0 | 1.21 | 0.63 | 0.0004 | 0.45 | 0.82 | 0.71 | 0.59 | 3.09 | 0.0001 | 0 | 0 | 0.02 | 0.004 |
| 3889144 | 0.7 | 0 | 1.13 | 0.64 | 0.0001 | 0.6 | 0.67 | 0.66 | 0.62 | 5.77 | 0 | 0 | 0 | 0 | 0 |
| 3889625 | 0.69 | 0 | 1.15 | 0.63 | 0.0002 | 0.58 | 0.68 | 0.66 | 0.61 | 5.32 | 0.0002 | 0 | 0 | 0 | 0 |
| 3897081 | 0.69 | 0 | 1.11 | 0.66 | 0 | 0.5 | 0.83 | 0.75 | 0.62 | 5.73 | 0 | 0 | 0 | 0.1 | 0.002 |
| 3902441 | 0.68 | 0 | 0.95 | 0.38 | 0.9999 | 0.47 | 0.3 | 0.41 | 0.35 | 6.22 | 0.0007 | 0.0001 | 0.0001 | 0.01 | 0.001 |
| 4026042 | 0.69 | 0 | 1.15 | 0.64 | 0.0001 | 0.61 | 0.66 | 0.65 | 0.63 | 7.8 | 0.0001 | 0 | 0 | 0.01 | 0.001 |

Performance of the 1,425 detected biomarkers across different metrics for the recurrence endpoint on the pooled discovery and validation cohort (n=199). See Example 2 for explanation of metrics.

TABLE 19

| | | | | | ACCU- | | | | | | | | |
|---|---|---|---|---|---|---|---|---|---|---|---|---|---|
| Affymetrix | | WILCOX | | | RACY | | | | | | KM | UVA HR | UVA OR |
| Probeset | | P- | | ACCU- | P- | SENSI- | SPECIF- | | | CUT | P- | P- | P- |
| ID | AUC | VALUE | MFD | RACY | VALUE | TIVITY | ICITY | PPV | NPV | OFF | VALUE | VALUE | VALUE |
| 2318654 | 0.65 | 0.0052 | 1.33 | 0.64 | 0.5397 | 0.7 | 0.6 | 0.5 | 0.78 | 7.85 | 0.001 | 0.0038 | 0.0052 |
| 2318755 | 0.63 | 0.0113 | 1.66 | 0.67 | 0.2627 | 0.6 | 0.71 | 0.54 | 0.75 | 5.57 | 0.0005 | 0.0043 | 0.007 |
| 2320062 | 0.65 | 0.0054 | 1.26 | 0.64 | 0.4669 | 0.6 | 0.67 | 0.51 | 0.74 | 5.23 | 0.0014 | 0.0043 | 0.0091 |
| 2321661 | 0.65 | 0.006 | 1.43 | 0.64 | 0.5397 | 0.72 | 0.59 | 0.5 | 0.79 | 6.52 | 0.0012 | 0.0042 | 0.0049 |
| 2322383 | 0.63 | 0.012 | 1.47 | 0.63 | 0.611 | 0.66 | 0.61 | 0.49 | 0.76 | 5.18 | 0.0017 | 0.0078 | 0.0127 |
| 2325809 | 0.62 | 0.0233 | 1.38 | 0.61 | 0.7405 | 0.62 | 0.61 | 0.48 | 0.74 | 6.06 | 0.0111 | 0.0139 | 0.019 |
| 2327494 | 0.63 | 0.0164 | 1.38 | 0.63 | 0.611 | 0.68 | 0.6 | 0.49 | 0.77 | 6.31 | 0.0023 | 0.0069 | 0.0185 |
| 2327865 | 0.64 | 0.0082 | 1.46 | 0.61 | 0.7405 | 0.66 | 0.59 | 0.48 | 0.75 | 5.34 | 0.0037 | 0.0031 | 0.0059 |
| 2328504 | 0.67 | 0.0013 | 1.4 | 0.67 | 0.2627 | 0.7 | 0.65 | 0.53 | 0.79 | 6.54 | 0.0002 | 0.0013 | 0.0012 |
| 2328507 | 0.68 | 0.0005 | 1.34 | 0.66 | 0.3262 | 0.47 | 0.77 | 0.54 | 0.72 | 5.36 | 0.0045 | 0.0019 | 0.0022 |
| 2329019 | 0.65 | 0.004 | 1.3 | 0.64 | 0.5397 | 0.64 | 0.63 | 0.5 | 0.75 | 6.44 | 0.0016 | 0.0042 | 0.0052 |
| 2329798 | 0.65 | 0.0049 | 1.53 | 0.64 | 0.5397 | 0.62 | 0.65 | 0.5 | 0.75 | 4.79 | 0.0055 | 0.0085 | 0.0079 |
| 2330704 | 0.62 | 0.023 | 1.35 | 0.6 | 0.8429 | 0.64 | 0.57 | 0.46 | 0.73 | 4.95 | 0.0143 | 0.0153 | 0.0189 |
| 2331419 | 0.65 | 0.0043 | 1.32 | 0.62 | 0.6786 | 0.47 | 0.71 | 0.48 | 0.7 | 4.58 | 0.0358 | 0.0118 | 0.0099 |
| 2331842 | 0.61 | 0.0405 | 1.33 | 0.64 | 0.5397 | 0.6 | 0.66 | 0.5 | 0.74 | 5.92 | 0.0044 | 0.0215 | 0.0302 |
| 2332192 | 0.67 | 0.0012 | 1.61 | 0.64 | 0.5397 | 0.51 | 0.71 | 0.5 | 0.72 | 5.14 | 0.0079 | 0.0023 | 0.0031 |
| 2332740 | 0.66 | 0.002 | 1.48 | 0.67 | 0.206 | 0.57 | 0.73 | 0.55 | 0.75 | 3.69 | 0.0004 | 0.001 | 0.0026 |
| 2334302 | 0.66 | 0.0028 | 1.33 | 0.67 | 0.2627 | 0.72 | 0.63 | 0.53 | 0.8 | 6.17 | 0.0001 | 0.0016 | 0.0021 |
| 2336960 | 0.66 | 0.003 | 1.31 | 0.64 | 0.4669 | 0.62 | 0.66 | 0.51 | 0.75 | 6.62 | 0.0011 | 0.0018 | 0.0028 |
| 2339310 | 0.64 | 0.0065 | 1.43 | 0.67 | 0.2627 | 0.55 | 0.73 | 0.54 | 0.74 | 4.64 | 0.0014 | 0.0039 | 0.0054 |
| 2342617 | 0.64 | 0.0104 | 1.3 | 0.61 | 0.7405 | 0.51 | 0.67 | 0.47 | 0.71 | 4.46 | 0.0632 | 0.0188 | 0.0159 |
| 2345634 | 0.66 | 0.0024 | 1.44 | 0.6 | 0.8429 | 0.64 | 0.57 | 0.46 | 0.73 | 4.69 | 0.0111 | 0.0009 | 0.0021 |
| 2345663 | 0.7 | 0.0001 | 1.29 | 0.64 | 0.4669 | 0.74 | 0.59 | 0.51 | 0.8 | 4.44 | 0.0009 | 0.0001 | 0.0003 |
| 2345666 | 0.6 | 0.0676 | 1.36 | 0.63 | 0.611 | 0.51 | 0.7 | 0.49 | 0.71 | 4.25 | 0.0106 | 0.0392 | 0.0587 |
| 2346911 | 0.63 | 0.0141 | 1.27 | 0.64 | 0.4669 | 0.55 | 0.7 | 0.51 | 0.73 | 7.17 | 0.0048 | 0.0092 | 0.0129 |
| 2346912 | 0.62 | 0.0189 | 1.37 | 0.61 | 0.7405 | 0.4 | 0.73 | 0.46 | 0.68 | 3.88 | 0.1237 | 0.0236 | 0.0198 |
| 2347108 | 0.64 | 0.0096 | 1.34 | 0.63 | 0.611 | 0.45 | 0.73 | 0.49 | 0.7 | 6.04 | 0.0286 | 0.0121 | 0.0141 |
| 2348819 | 0.66 | 0.0029 | 1.37 | 0.65 | 0.3949 | 0.64 | 0.66 | 0.52 | 0.76 | 5.97 | 0.0009 | 0.0031 | 0.0035 |
| 2350305 | 0.62 | 0.0292 | 1.34 | 0.64 | 0.5397 | 0.62 | 0.65 | 0.5 | 0.75 | 5.19 | 0.0029 | 0.0131 | 0.0214 |
| 2350371 | 0.65 | 0.0043 | 1.25 | 0.62 | 0.6786 | 0.38 | 0.76 | 0.47 | 0.68 | 4.47 | 0.0896 | 0.0057 | 0.0056 |
| 2352268 | 0.69 | 0.0004 | 1.34 | 0.66 | 0.3262 | 0.64 | 0.67 | 0.53 | 0.76 | 6.24 | 0.0004 | 0.0005 | 0.001 |
| 2353492 | 0.63 | 0.017 | 1.27 | 0.64 | 0.5397 | 0.62 | 0.65 | 0.5 | 0.75 | 6.15 | 0.0021 | 0.0093 | 0.0192 |
| 2358275 | 0.59 | 0.0819 | 1.25 | 0.63 | 0.611 | 0.53 | 0.68 | 0.49 | 0.72 | 4.26 | 0.0187 | 0.0715 | 0.0886 |
| 2358923 | 0.64 | 0.0096 | 1.44 | 0.65 | 0.3949 | 0.7 | 0.62 | 0.52 | 0.78 | 6.94 | 0.0005 | 0.007 | 0.0096 |
| 2359857 | 0.67 | 0.0017 | 1.33 | 0.66 | 0.3262 | 0.66 | 0.66 | 0.53 | 0.77 | 4.36 | 0.0006 | 0.005 | 0.0044 |
| 2360078 | 0.66 | 0.0022 | 1.29 | 0.64 | 0.4669 | 0.55 | 0.7 | 0.51 | 0.73 | 7.34 | 0.0024 | 0.0011 | 0.0033 |
| 2360119 | 0.66 | 0.0024 | 1.32 | 0.67 | 0.206 | 0.68 | 0.67 | 0.54 | 0.79 | 7.4 | 0.0001 | 0.0021 | 0.0027 |
| 2360120 | 0.66 | 0.0021 | 1.25 | 0.62 | 0.6786 | 0.62 | 0.62 | 0.48 | 0.74 | 7.06 | 0.0072 | 0.0009 | 0.0016 |
| 2360340 | 0.63 | 0.0115 | 1.28 | 0.67 | 0.206 | 0.55 | 0.74 | 0.55 | 0.74 | 5.72 | 0.0007 | 0.0066 | 0.009 |
| 2360716 | 0.63 | 0.0141 | 1.28 | 0.61 | 0.7405 | 0.77 | 0.52 | 0.48 | 0.8 | 7.12 | 0.0016 | 0.0166 | 0.0204 |
| 2360962 | 0.68 | 0.001 | 1.36 | 0.65 | 0.3949 | 0.6 | 0.68 | 0.52 | 0.75 | 5.51 | 0.0008 | 0.0006 | 0.0013 |
| 2361031 | 0.58 | 0.1168 | 1.15 | 0.61 | 0.7405 | 0.45 | 0.71 | 0.47 | 0.69 | 3.33 | 0.06 | 0.1638 | 0.1501 |
| 2363064 | 0.67 | 0.0012 | 1.29 | 0.61 | 0.7405 | 0.66 | 0.59 | 0.48 | 0.75 | 6.45 | 0.0075 | 0.001 | 0.0012 |
| 2363334 | 0.62 | 0.0233 | 1.29 | 0.57 | 0.9388 | 0.53 | 0.6 | 0.43 | 0.69 | 5.01 | 0.0894 | 0.0173 | 0.0279 |
| 2363956 | 0.68 | 0.0006 | 1.28 | 0.67 | 0.206 | 0.51 | 0.77 | 0.56 | 0.73 | 4.57 | 0.0017 | 0.0055 | 0.0064 |
| 2365991 | 0.62 | 0.0187 | 1.61 | 0.61 | 0.7405 | 0.6 | 0.62 | 0.47 | 0.73 | 4.56 | 0.0113 | 0.0352 | 0.0361 |
| 2367178 | 0.67 | 0.0016 | 1.6 | 0.64 | 0.4669 | 0.68 | 0.62 | 0.51 | 0.77 | 4.92 | 0.0008 | 0.0019 | 0.0025 |
| 2367214 | 0.64 | 0.0094 | 1.46 | 0.61 | 0.7405 | 0.66 | 0.59 | 0.48 | 0.75 | 4.86 | 0.0047 | 0.0077 | 0.0097 |
| 2367236 | 0.63 | 0.0177 | 1.32 | 0.63 | 0.611 | 0.66 | 0.61 | 0.49 | 0.76 | 5.6 | 0.0024 | 0.0109 | 0.0171 |
| 2369830 | 0.68 | 0.0009 | 1.47 | 0.66 | 0.3262 | 0.66 | 0.66 | 0.53 | 0.77 | 4.54 | 0.0004 | 0.0012 | 0.0018 |
| 2370014 | 0.65 | 0.0038 | 1.26 | 0.61 | 0.7405 | 0.62 | 0.61 | 0.48 | 0.74 | 4.18 | 0.0105 | 0.001 | 0.004 |
| 2371291 | 0.66 | 0.0026 | 1.37 | 0.63 | 0.611 | 0.55 | 0.67 | 0.49 | 0.72 | 5.26 | 0.0078 | 0.0022 | 0.0059 |
| 2371339 | 0.64 | 0.0078 | 1.36 | 0.63 | 0.611 | 0.55 | 0.67 | 0.49 | 0.72 | 4.45 | 0.0111 | 0.0198 | 0.0222 |
| 2371796 | 0.67 | 0.0016 | 1.31 | 0.63 | 0.611 | 0.51 | 0.7 | 0.49 | 0.71 | 5.7 | 0.0134 | 0.0023 | 0.0036 |
| 2373002 | 0.64 | 0.0076 | 1.46 | 0.66 | 0.3262 | 0.57 | 0.71 | 0.53 | 0.74 | 4.06 | 0.0005 | 0.0133 | 0.016 |
| 2375035 | 0.66 | 0.002 | 1.4 | 0.65 | 0.3949 | 0.53 | 0.72 | 0.52 | 0.73 | 4.75 | 0.0033 | 0.002 | 0.0027 |
| 2375037 | 0.69 | 0.0004 | 1.27 | 0.67 | 0.2627 | 0.66 | 0.67 | 0.53 | 0.77 | 6.88 | 0.0002 | 0.0004 | 0.0006 |
| 2375675 | 0.65 | 0.0056 | 1.35 | 0.62 | 0.6786 | 0.7 | 0.57 | 0.49 | 0.77 | 4.68 | 0.003 | 0.0091 | 0.0072 |
| 2375780 | 0.64 | 0.0079 | 1.44 | 0.62 | 0.6786 | 0.62 | 0.62 | 0.48 | 0.74 | 5.94 | 0.006 | 0.006 | 0.008 |
| 2375850 | 0.68 | 0.0009 | 1.27 | 0.67 | 0.206 | 0.66 | 0.68 | 0.54 | 0.78 | 6.85 | 0.0001 | 0.0005 | 0.001 |
| 2375890 | 0.65 | 0.0053 | 1.41 | 0.6 | 0.7956 | 0.68 | 0.56 | 0.47 | 0.75 | 6.85 | 0.0055 | 0.0038 | 0.0047 |
| 2377473 | 0.7 | 0.0002 | 1.43 | 0.6 | 0.7956 | 0.66 | 0.57 | 0.47 | 0.75 | 4.47 | 0.0072 | 0.0002 | 0.0005 |
| 2377508 | 0.68 | 0.0009 | 1.63 | 0.63 | 0.611 | 0.68 | 0.6 | 0.49 | 0.77 | 5.88 | 0.0016 | 0.0005 | 0.0011 |
| 2378615 | 0.61 | 0.0303 | 1.35 | 0.61 | 0.7405 | 0.51 | 0.67 | 0.47 | 0.71 | 3.71 | 0.0266 | 0.0335 | 0.0664 |
| 2379787 | 0.67 | 0.0017 | 1.36 | 0.61 | 0.7405 | 0.77 | 0.52 | 0.48 | 0.8 | 4.65 | 0.0021 | 0.0015 | 0.0019 |
| 2383763 | 0.64 | 0.0069 | 1.3 | 0.63 | 0.611 | 0.66 | 0.61 | 0.49 | 0.76 | 6.36 | 0.0026 | 0.0055 | 0.0087 |
| 2389076 | 0.66 | 0.0026 | 1.38 | 0.65 | 0.3949 | 0.57 | 0.7 | 0.52 | 0.74 | 4.92 | 0.0014 | 0.0017 | 0.0032 |
| 2389079 | 0.68 | 0.0006 | 1.36 | 0.67 | 0.206 | 0.49 | 0.78 | 0.56 | 0.73 | 4.81 | 0.0007 | 0.0007 | 0.0012 |
| 2389084 | 0.63 | 0.0185 | 1.34 | 0.67 | 0.2627 | 0.49 | 0.77 | 0.55 | 0.72 | 5.29 | 0.0014 | 0.0051 | 0.0095 |
| 2389086 | 0.64 | 0.0066 | 1.29 | 0.64 | 0.5397 | 0.55 | 0.68 | 0.5 | 0.73 | 5.34 | 0.0075 | 0.0083 | 0.0102 |
| 2389817 | 0.68 | 0.0007 | 1.33 | 0.66 | 0.3262 | 0.57 | 0.71 | 0.53 | 0.74 | 7.11 | 0.0016 | 0.0012 | 0.0018 |
| 2394560 | 0.64 | 0.0094 | 1.73 | 0.6 | 0.8479 | 0.62 | 0.59 | 0.46 | 0.73 | 5.1 | 0.0164 | 0.0088 | 0.0103 |
| 2395192 | 0.66 | 0.0025 | 1.49 | 0.67 | 0.2627 | 0.55 | 0.73 | 0.54 | 0.74 | 6.47 | 0.0004 | 0.0005 | 0.0016 |
| 2395195 | 0.67 | 0.0016 | 1.26 | 0.61 | 0.7405 | 0.62 | 0.61 | 0.48 | 0.74 | 5.06 | 0.0119 | 0.0019 | 0.0033 |
| 2396421 | 0.63 | 0.0112 | 1.33 | 0.61 | 0.7405 | 0.57 | 0.63 | 0.47 | 0.72 | 4.36 | 0.0155 | 0.0106 | 0.0129 |
| 2400180 | 0.64 | 0.0086 | 1.32 | 0.6 | 0.8429 | 0.57 | 0.61 | 0.46 | 0.71 | 5.75 | 0.0214 | 0.0027 | 0.0075 |
| 2400323 | 0.63 | 0.013 | 1.3 | 0.6 | 0.8429 | 0.45 | 0.68 | 0.45 | 0.68 | 3.94 | 0.1368 | 0.0079 | 0.0162 |
| 2401277 | 0.73 | 0 | 1.39 | 0.7 | 0.0837 | 0.62 | 0.74 | 0.58 | 0.77 | 2.96 | 0 | 0 | 0.0002 |
| 2401288 | 0.69 | 0.0005 | 1.27 | 0.63 | 0.611 | 0.72 | 0.57 | 0.49 | 0.78 | 5.94 | 0.0008 | 0.0003 | 0.0005 |
| 2401350 | 0.69 | 0.0005 | 1.52 | 0.64 | 0.5397 | 0.66 | 0.62 | 0.5 | 0.76 | 5.08 | 0.0009 | 0.0004 | 0.0007 |
| 2401368 | 0.64 | 0.0106 | 1.31 | 0.69 | 0.1164 | 0.43 | 0.84 | 0.61 | 0.72 | 5.17 | 0.0005 | 0.0018 | 0.0038 |
| 2402210 | 0.66 | 0.003 | 1.22 | 0.64 | 0.4669 | 0.7 | 0.61 | 0.51 | 0.78 | 5.56 | 0.0006 | 0.0046 | 0.0073 |
| 2405379 | 0.65 | 0.0037 | 1.4 | 0.64 | 0.4669 | 0.64 | 0.65 | 0.51 | 0.76 | 5.51 | 0.0013 | 0.0037 | 0.0051 |
| 2406094 | 0.64 | 0.0064 | 1.43 | 0.65 | 0.3949 | 0.64 | 0.66 | 0.52 | 0.76 | 6.24 | 0.0005 | 0.0041 | 0.0073 |
| 2406148 | 0.65 | 0.0059 | 1.44 | 0.64 | 0.4669 | 0.74 | 0.59 | 0.51 | 0.8 | 6.23 | 0.0003 | 0.0047 | 0.0048 |
| 2407134 | 0.63 | 0.0177 | 1.36 | 0.64 | 0.4669 | 0.57 | 0.68 | 0.51 | 0.74 | 4.7 | 0.0021 | 0.0096 | 0.0161 |
| 2408880 | 0.62 | 0.0205 | 1.33 | 0.6 | 0.8429 | 0.53 | 0.63 | 0.45 | 0.7 | 4.7 | 0.0531 | 0.0151 | 0.0246 |
| 2410112 | 0.65 | 0.0042 | 1.35 | 0.63 | 0.611 | 0.72 | 0.57 | 0.49 | 0.78 | 5.4 | 0.0013 | 0.003 | 0.0027 |
| 2410528 | 0.63 | 0.0126 | 1.26 | 0.6 | 0.7956 | 0.79 | 0.5 | 0.47 | 0.8 | 6.06 | 0.001 | 0.0094 | 0.0138 |
| 2412669 | 0.64 | 0.0092 | 1.32 | 0.63 | 0.611 | 0.55 | 0.67 | 0.49 | 0.72 | 4.75 | 0.0104 | 0.0112 | 0.0112 |
| 2413580 | 0.64 | 0.008 | 1.3 | 0.64 | 0.4669 | 0.57 | 0.68 | 0.51 | 0.74 | 5.02 | 0.0023 | 0.0102 | 0.0125 |
| 2414030 | 0.68 | 0.001 | 1.52 | 0.64 | 0.4669 | 0.62 | 0.66 | 0.51 | 0.75 | 4.78 | 0.0079 | 0.002 | 0.0019 |
| 2414960 | 0.62 | 0.0194 | 1.36 | 0.6 | 0.8429 | 0.6 | 0.6 | 0.46 | 0.72 | 4.59 | 0.0172 | 0.0064 | 0.0202 |
| 2417788 | 0.64 | 0.0075 | 1.42 | 0.64 | 0.4669 | 0.62 | 0.66 | 0.51 | 0.75 | 4.86 | 0.0005 | 0.0063 | 0.0107 |
| 2418028 | 0.66 | 0.002 | 1.29 | 0.68 | 0.1571 | 0.62 | 0.72 | 0.56 | 0.77 | 5.81 | 0.0002 | 0.0024 | 0.0028 |
| 2419242 | 0.69 | 0.0005 | 1.34 | 0.63 | 0.611 | 0.43 | 0.74 | 0.49 | 0.69 | 4.65 | 0.0341 | 0.0014 | 0.0018 |
| 2419247 | 0.6 | 0.0698 | 1.25 | 0.64 | 0.5397 | 0.49 | 0.72 | 0.5 | 0.71 | 5.37 | 0.0119 | 0.0363 | 0.0436 |
| 2419276 | 0.67 | 0.0016 | 1.31 | 0.64 | 0.4669 | 0.7 | 0.61 | 0.51 | 0.78 | 5.71 | 0.0004 | 0.0013 | 0.0024 |
| 2420740 | 0.65 | 0.0048 | 1.54 | 0.6 | 0.7956 | 0.53 | 0.65 | 0.46 | 0.71 | 3.96 | 0.0525 | 0.0024 | 0.0031 |
| 2420839 | 0.65 | 0.0036 | 1.34 | 0.66 | 0.3262 | 0.47 | 0.77 | 0.54 | 0.72 | 4.98 | 0.0043 | 0.0014 | 0.0027 |
| 2421273 | 0.67 | 0.0012 | 1.33 | 0.69 | 0.1164 | 0.64 | 0.72 | 0.57 | 0.78 | 6.06 | 0 | 0.0009 | 0.0014 |
| 2421274 | 0.64 | 0.0068 | 1.31 | 0.65 | 0.3949 | 0.6 | 0.68 | 0.52 | 0.75 | 4.52 | 0.0016 | 0.0095 | 0.0146 |
| 2421932 | 0.64 | 0.0072 | 1.31 | 0.61 | 0.7405 | 0.74 | 0.54 | 0.48 | 0.79 | 6.5 | 0.0028 | 0.011 | 0.0083 |
| 2422519 | 0.65 | 0.0044 | 1.41 | 0.64 | 0.4669 | 0.57 | 0.68 | 0.51 | 0.74 | 4.8 | 0.0038 | 0.0041 | 0.0064 |
| 2423200 | 0.65 | 0.0062 | 1.37 | 0.65 | 0.3949 | 0.68 | 0.63 | 0.52 | 0.78 | 6.89 | 0.0005 | 0.0027 | 0.0042 |
| 2427009 | 0.6 | 0.05 | 1.51 | 0.6 | 0.7956 | 0.53 | 0.65 | 0.46 | 0.71 | 4.01 | 0.0398 | 0.0192 | 0.0271 |
| 2427501 | 0.65 | 0.0046 | 1.65 | 0.65 | 0.3949 | 0.68 | 0.63 | 0.52 | 0.78 | 4.76 | 0.0006 | 0.0024 | 0.0045 |
| 2427978 | 0.66 | 0.0021 | 1.43 | 0.66 | 0.3262 | 0.64 | 0.67 | 0.53 | 0.76 | 6.15 | 0.0007 | 0.0014 | 0.0016 |
| 2428394 | 0.68 | 0.0007 | 1.36 | 0.71 | 0.0583 | 0.68 | 0.72 | 0.58 | 0.8 | 6.19 | 0 | 0.0007 | 0.0009 |
| 2429071 | 0.67 | 0.0016 | 1.26 | 0.62 | 0.6786 | 0.45 | 0.72 | 0.48 | 0.69 | 4.47 | 0.0691 | 0.0068 | 0.0059 |
| 2429119 | 0.69 | 0.0003 | 1.39 | 0.67 | 0.206 | 0.68 | 0.67 | 0.54 | 0.79 | 5.43 | 0.0001 | 0.0004 | 0.0006 |
| 2439168 | 0.63 | 0.0134 | 1.43 | 0.57 | 0.9388 | 0.47 | 0.63 | 0.42 | 0.68 | 3.82 | 0.2306 | 0.03 | 0.0506 |
| 2429282 | 0.69 | 0.0004 | 1.38 | 0.67 | 0.206 | 0.57 | 0.73 | 0.55 | 0.75 | 4.96 | 0.0003 | 0.0008 | 0.001 |
| 2429309 | 0.67 | 0.0015 | 1.37 | 0.64 | 0.5397 | 0.68 | 0.61 | 0.5 | 0.77 | 6.53 | 0.0012 | 0.0008 | 0.0016 |
| 2429310 | 0.66 | 0.0022 | 1.66 | 0.62 | 0.6786 | 0.68 | 0.59 | 0.48 | 0.76 | 5.42 | 0.003 | 0.0012 | 0.0021 |
| 2433376 | 0.63 | 0.0149 | 1.33 | 0.64 | 0.4669 | 0.64 | 0.65 | 0.51 | 0.76 | 4.99 | 0.0012 | 0.0188 | 0.0224 |
| 2434721 | 0.63 | 0.0121 | 1.34 | 0.58 | 0.914 | 0.53 | 0.61 | 0.44 | 0.69 | 5.46 | 0.1464 | 0.016 | 0.0175 |
| 2435391 | 0.65 | 0.0049 | 1.36 | 0.64 | 0.4669 | 0.53 | 0.71 | 0.51 | 0.73 | 6.56 | 0.0035 | 0.0041 | 0.0079 |
| 2437095 | 0.63 | 0.0139 | 1.32 | 0.58 | 0.914 | 0.64 | 0.55 | 0.45 | 0.73 | 6.33 | 0.0342 | 0.0142 | 0.0147 |
| 2437537 | 0.6 | 0.0541 | 1.3 | 0.61 | 0.7405 | 0.45 | 0.71 | 0.47 | 0.69 | 5.24 | 0.0806 | 0.0959 | 0.0881 |
| 2437538 | 0.68 | 0.0008 | 1.51 | 0.62 | 0.6786 | 0.72 | 0.56 | 0.49 | 0.78 | 6.29 | 0.0019 | 0.0011 | 0.0009 |
| 2437680 | 0.65 | 0.0062 | 1.37 | 0.65 | 0.3949 | 0.62 | 0.67 | 0.52 | 0.75 | 4.52 | 0.0011 | 0.0171 | 0.0171 |
| 2437775 | 0.62 | 0.0261 | 1.31 | 0.64 | 0.5397 | 0.49 | 0.72 | 0.5 | 0.71 | 4.15 | 0.0219 | 0.0254 | 0.0273 |
| 2439041 | 0.6 | 0.0511 | 1.28 | 0.61 | 0.7405 | 0.55 | 0.65 | 0.47 | 0.72 | 4.52 | 0.0389 | 0.0635 | 0.0547 |
| 2440149 | 0.64 | 0.0082 | 1.32 | 0.61 | 0.7405 | 0.55 | 0.65 | 0.47 | 0.72 | 4.93 | 0.0155 | 0.0091 | 0.0159 |
| 2440513 | 0.67 | 0.001 | 1.33 | 0.62 | 0.6786 | 0.74 | 0.55 | 0.49 | 0.79 | 8.24 | 0.0009 | 0.0019 | 0.0026 |
| 2442702 | 0.62 | 0.0233 | 1.3 | 0.64 | 0.4669 | 0.51 | 0.72 | 0.51 | 0.72 | 5.53 | 0.0042 | 0.0145 | 0.0234 |
| 2443917 | 0.66 | 0.0024 | 1.34 | 0.65 | 0.3949 | 0.55 | 0.71 | 0.52 | 0.73 | 4.71 | 0.003 | 0.0026 | 0.0034 |
| 2444488 | 0.62 | 0.0236 | 1.32 | 0.65 | 0.3949 | 0.49 | 0.74 | 0.52 | 0.72 | 3.92 | 0.0049 | 0.0118 | 0.0235 |
| 2444489 | 0.62 | 0.0197 | 1.32 | 0.6 | 0.7956 | 0.36 | 0.74 | 0.45 | 0.67 | 4.42 | 0.1867 | 0.0227 | 0.0376 |
| 2448289 | 0.61 | 0.0391 | 1.28 | 0.64 | 0.5397 | 0.43 | 0.76 | 0.5 | 0.7 | 4.08 | 0.0437 | 0.0535 | 0.051 |
| 2448313 | 0.66 | 0.0032 | 1.47 | 0.64 | 0.5397 | 0.53 | 0.7 | 0.5 | 0.72 | 4.09 | 0.0079 | 0.0034 | 0.0061 |
| 2450669 | 0.64 | 0.0072 | 1.49 | 0.6 | 0.8429 | 0.6 | 0.6 | 0.46 | 0.72 | 5.06 | 0.0236 | 0.0053 | 0.0068 |
| 2451314 | 0.63 | 0.0137 | 1.36 | 0.63 | 0.611 | 0.6 | 0.65 | 0.49 | 0.74 | 4.55 | 0.0069 | 0.0025 | 0.0074 |
| 2452652 | 0.67 | 0.0011 | 1.43 | 0.66 | 0.3262 | 0.72 | 0.6 | 0.52 | 0.8 | 6.12 | 0.0001 | 0.001 | 0.0012 |
| 2452655 | 0.63 | 0.0121 | 1.5 | 0.65 | 0.3949 | 0.55 | 0.71 | 0.52 | 0.73 | 4.93 | 0.0018 | 0.007 | 0.0169 |
| 2457626 | 0.68 | 0.0005 | 1.3 | 0.67 | 0.2627 | 0.51 | 0.76 | 0.55 | 0.73 | 5.73 | 0.0015 | 0.0011 | 0.0013 |
| 2458075 | 0.62 | 0.0219 | 1.51 | 0.64 | 0.5397 | 0.57 | 0.67 | 0.5 | 0.73 | 5.28 | 0.0031 | 0.0125 | 0.0211 |
| 2458381 | 0.62 | 0.0292 | 1.2 | 0.58 | 0.914 | 0.47 | 0.65 | 0.43 | 0.68 | 4.9 | 0.2056 | 0.0113 | 0.0215 |
| 2458503 | 0.66 | 0.0026 | 1.33 | 0.64 | 0.5397 | 0.68 | 0.61 | 0.5 | 0.77 | 5.73 | 0.0014 | 0.0015 | 0.0023 |
| 2459655 | 0.65 | 0.0053 | 1.23 | 0.62 | 0.6786 | 0.47 | 0.71 | 0.48 | 0.7 | 6.48 | 0.0268 | 0.0041 | 0.0067 |
| 2461827 | 0.67 | 0.0015 | 1.32 | 0.67 | 0.2627 | 0.6 | 0.71 | 0.54 | 0.75 | 5.09 | 0.0004 | 0.002 | 0.003 |
| 2461828 | 0.67 | 0.0018 | 1.35 | 0.64 | 0.4669 | 0.66 | 0.63 | 0.51 | 0.76 | 5.29 | 0.0014 | 0.0024 | 0.0028 |
| 2462178 | 0.64 | 0.0074 | 1.34 | 0.58 | 0.914 | 0.55 | 0.6 | 0.44 | 0.7 | 4.45 | 0.0924 | 0.0027 | 0.0068 |
| 2464496 | 0.69 | 0.0005 | 1.58 | 0.67 | 0.2627 | 0.68 | 0.66 | 0.53 | 0.78 | 5.38 | 0.0002 | 0.0004 | 0.0008 |
| 2464505 | 0.68 | 0.001 | 1.35 | 0.67 | 0.206 | 0.68 | 0.67 | 0.54 | 0.79 | 6.3 | 0.0001 | 0.0007 | 0.0011 |
| 2464514 | 0.66 | 0.0025 | 1.32 | 0.64 | 0.4669 | 0.66 | 0.63 | 0.51 | 0.76 | 5.72 | 0.001 | 0.0024 | 0.0028 |
| 2464537 | 0.66 | 0.0024 | 1.41 | 0.67 | 0.206 | 0.62 | 0.71 | 0.55 | 0.76 | 5.7 | 0.0001 | 0.0012 | 0.0021 |
| 2468935 | 0.62 | 0.0271 | 1.24 | 0.6 | 0.8429 | 0.47 | 0.67 | 0.45 | 0.69 | 4.85 | 0.1455 | 0.0219 | 0.0241 |
| 2469568 | 0.7 | 0.0002 | 1.37 | 0.69 | 0.1164 | 0.62 | 0.73 | 0.57 | 0.77 | 4.27 | 0.0001 | 0.0005 | 0.001 |
| 2473241 | 0.68 | 0.0005 | 1.25 | 0.64 | 0.4669 | 0.53 | 0.71 | 0.51 | 0.73 | 4.8 | 0.008 | 0.0015 | 0.0023 |
| 2473624 | 0.66 | 0.002 | 1.66 | 0.64 | 0.5397 | 0.66 | 0.62 | 0.5 | 0.76 | 4.93 | 0.0018 | 0.0013 | 0.0019 |
| 2474147 | 0.61 | 0.0369 | 1.36 | 0.58 | 0.914 | 0.66 | 0.54 | 0.45 | 0.73 | 6.87 | 0.0257 | 0.0254 | 0.0377 |
| 2474700 | 0.67 | 0.0016 | 1.36 | 0.64 | 0.5397 | 0.72 | 0.59 | 0.5 | 0.79 | 5.15 | 0.0005 | 0.0018 | 0.0023 |
| 2474770 | 0.69 | 0.0004 | 1.42 | 0.68 | 0.1571 | 0.7 | 0.67 | 0.55 | 0.8 | 6.05 | 0 | 0.0004 | 0.0005 |
| 2475647 | 0.6 | 0.0529 | 1.33 | 0.61 | 0.7405 | 0.49 | 0.68 | 0.47 | 0.7 | 4.98 | 0.0592 | 0.0594 | 0.0558 |
| 2476316 | 0.7 | 0.0002 | 1.26 | 0.65 | 0.3949 | 0.64 | 0.66 | 0.52 | 0.76 | 4.67 | 0.001 | 0.0007 | 0.0013 |
| 2476408 | 0.65 | 0.0039 | 1.53 | 0.64 | 0.5397 | 0.6 | 0.66 | 0.5 | 0.74 | 5.02 | 0.0034 | 0.002 | 0.0036 |
| 2477341 | 0.64 | 0.0097 | 1.43 | 0.6 | 0.7956 | 0.6 | 0.61 | 0.47 | 0.72 | 5.4 | 0.019 | 0.0104 | 0.0102 |
| 2478839 | 0.65 | 0.0049 | 1.32 | 0.65 | 0.3949 | 0.66 | 0.65 | 0.52 | 0.77 | 6.62 | 0.0008 | 0.0028 | 0.0033 |
| 2479729 | 0.67 | 0.0016 | 1.38 | 0.61 | 0.7405 | 0.72 | 0.55 | 0.48 | 0.78 | 5.13 | 0.003 | 0.0011 | 0.0012 |
| 2480989 | 0.69 | 0.0004 | 1.36 | 0.64 | 0.4669 | 0.47 | 0.74 | 0.51 | 0.71 | 4.92 | 0.0068 | 0.0002 | 0.0006 |
| 2481178 | 0.66 | 0.0032 | 1.43 | 0.67 | 0.2627 | 0.64 | 0.68 | 0.54 | 0.77 | 5.04 | 0.0005 | 0.0024 | 0.0026 |
| 2482593 | 0.66 | 0.0033 | 1.32 | 0.63 | 0.611 | 0.53 | 0.68 | 0.49 | 0.72 | 5.54 | 0.0104 | 0.0035 | 0.0051 |
| 2482624 | 0.67 | 0.0011 | 1.28 | 0.62 | 0.6786 | 0.72 | 0.56 | 0.49 | 0.78 | 7.06 | 0.0017 | 0.0009 | 0.0012 |
| 2483047 | 0.65 | 0.0057 | 1.51 | 0.59 | 0.8822 | 0.53 | 0.62 | 0.45 | 0.7 | 5.04 | 0.0678 | 0.0075 | 0.011 |
| 2484558 | 0.67 | 0.0013 | 1.39 | 0.67 | 0.206 | 0.49 | 0.78 | 0.56 | 0.73 | 4.17 | 0.0011 | 0.0069 | 0.0071 |
| 2484807 | 0.67 | 0.0014 | 1.32 | 0.59 | 0.8822 | 0.77 | 0.49 | 0.46 | 0.78 | 6.35 | 0.0048 | 0.0017 | 0.0016 |
| 2485214 | 0.64 | 0.008 | 1.39 | 0.62 | 0.6786 | 0.6 | 0.63 | 0.48 | 0.73 | 5.47 | 0.0091 | 0.0079 | 0.0085 |
| 2485276 | 0.63 | 0.0121 | 1.25 | 0.6 | 0.8429 | 0.49 | 0.66 | 0.45 | 0.69 | 4.49 | 0.0798 | 0.0433 | 0.0454 |
| 2487216 | 0.63 | 0.0113 | 1.49 | 0.63 | 0.611 | 0.6 | 0.65 | 0.49 | 0.74 | 5.06 | 0.0075 | 0.0064 | 0.0112 |
| 2487645 | 0.67 | 0.0015 | 1.34 | 0.67 | 0.206 | 0.62 | 0.71 | 0.55 | 0.76 | 5.91 | 0.0001 | 0.0018 | 0.0024 |
| 2488150 | 0.65 | 0.0058 | 1.21 | 0.61 | 0.7405 | 0.6 | 0.62 | 0.47 | 0.73 | 5.01 | 0.0208 | 0.0063 | 0.0102 |
| 2489030 | 0.64 | 0.0071 | 1.43 | 0.64 | 0.5397 | 0.6 | 0.66 | 0.5 | 0.74 | 5.64 | 0.004 | 0.0099 | 0.0075 |
| 2489435 | 0.67 | 0.0014 | 1.32 | 0.63 | 0.611 | 0.64 | 0.62 | 0.49 | 0.75 | 6.14 | 0.0036 | 0.002 | 0.0019 |
| 2491645 | 0.65 | 0.0046 | 1.3 | 0.64 | 0.5397 | 0.66 | 0.62 | 0.5 | 0.76 | 6.43 | 0.0014 | 0.0042 | 0.0048 |
| 2492101 | 0.65 | 0.0057 | 1.24 | 0.61 | 0.7405 | 0.62 | 0.61 | 0.48 | 0.74 | 3.51 | 0.0114 | 0.0024 | 0.0087 |
| 2492833 | 0.61 | 0.0415 | 1.38 | 0.6 | 0.7956 | 0.53 | 0.65 | 0.46 | 0.71 | 5.89 | 0.0251 | 0.0207 | 0.0408 |
| 2495140 | 0.66 | 0.002 | 1.53 | 0.64 | 0.5397 | 0.68 | 0.61 | 0.5 | 0.77 | 4.83 | 0.0015 | 0.0015 | 0.0021 |
| 2498988 | 0.66 | 0.003 | 1.46 | 0.65 | 0.3949 | 0.53 | 0.72 | 0.52 | 0.73 | 4.54 | 0.0042 | 0.0041 | 0.0073 |
| 2507241 | 0.68 | 0.0008 | 1.5 | 0.67 | 0.2627 | 0.57 | 0.72 | 0.54 | 0.75 | 5.06 | 0.0008 | 0.0013 | 0.0017 |
| 2507514 | 0.66 | 0.0035 | 1.37 | 0.64 | 0.4669 | 0.66 | 0.63 | 0.51 | 0.76 | 6.06 | 0.0016 | 0.0032 | 0.0051 |
| 2507533 | 0.67 | 0.0017 | 1.38 | 0.66 | 0.3262 | 0.68 | 0.65 | 0.52 | 0.78 | 6.67 | 0.0002 | 0.001 | 0.0016 |
| 2512351 | 0.65 | 0.0042 | 1.31 | 0.65 | 0.3949 | 0.66 | 0.65 | 0.52 | 0.77 | 4.43 | 0.0009 | 0.01 | 0.0116 |
| 2512363 | 0.64 | 0.0068 | 1.55 | 0.63 | 0.611 | 0.57 | 0.66 | 0.49 | 0.73 | 3.74 | 0.0081 | 0.0036 | 0.0051 |
| 2512959 | 0.64 | 0.009 | 1.26 | 0.59 | 0.8822 | 0.55 | 0.61 | 0.45 | 0.7 | 3.88 | 0.0675 | 0.0218 | 0.0201 |
| 2515085 | 0.64 | 0.0098 | 1.33 | 0.64 | 0.4669 | 0.66 | 0.63 | 0.51 | 0.76 | 6.98 | 0.0009 | 0.0051 | 0.0081 |
| 2517330 | 0.66 | 0.003 | 1.41 | 0.67 | 0.206 | 0.45 | 0.8 | 0.57 | 0.72 | 4.39 | 0.0013 | 0.0051 | 0.0069 |
| 2518193 | 0.68 | 0.0007 | 1.27 | 0.62 | 0.6786 | 0.77 | 0.54 | 0.49 | 0.8 | 5.52 | 0.001 | 0.0005 | 0.0009 |
| 2518707 | 0.64 | 0.0083 | 1.47 | 0.6 | 0.7956 | 0.57 | 0.62 | 0.47 | 0.72 | 5.24 | 0.0156 | 0.0031 | 0.0087 |
| 2525095 | 0.69 | 0.0004 | 1.37 | 0.67 | 0.206 | 0.6 | 0.72 | 0.55 | 0.76 | 5.7 | 0.0002 | 0.0005 | 0.0012 |
| 2527206 | 0.67 | 0.0015 | 1.32 | 0.6 | 0.8429 | 0.72 | 0.52 | 0.47 | 0.77 | 8.07 | 0.0048 | 0.0016 | 0.002 |
| 2527635 | 0.68 | 0.0005 | 1.36 | 0.61 | 0.7405 | 0.53 | 0.66 | 0.47 | 0.71 | 5.16 | 0.0273 | 0.0008 | 0.0011 |
| 2532844 | 0.63 | 0.0153 | 1.25 | 0.64 | 0.5397 | 0.49 | 0.72 | 0.5 | 0.71 | 4.77 | 0.0142 | 0.0131 | 0.0184 |
| 2534447 | 0.67 | 0.0013 | 1.42 | 0.69 | 0.1164 | 0.55 | 0.77 | 0.58 | 0.75 | 5.8 | 0.0001 | 0.0021 | 0.0025 |
| 2537112 | 0.64 | 0.0064 | 1.36 | 0.64 | 0.5397 | 0.66 | 0.62 | 0.5 | 0.76 | 6.32 | 0.0013 | 0.0042 | 0.0081 |
| 2539775 | 0.68 | 0.0008 | 1.46 | 0.67 | 0.206 | 0.51 | 0.77 | 0.56 | 0.73 | 4.74 | 0.0007 | 0.0011 | 0.002 |
| 2540221 | 0.64 | 0.01 | 1.48 | 0.63 | 0.611 | 0.66 | 0.61 | 0.49 | 0.76 | 6.42 | 0.0035 | 0.0057 | 0.007 |
| 2544180 | 0.66 | 0.0031 | 1.37 | 0.64 | 0.4669 | 0.66 | 0.63 | 0.51 | 0.76 | 6.73 | 0.001 | 0.0024 | 0.0026 |
| 2544185 | 0.6 | 0.0573 | 1.27 | 0.6 | 0.8429 | 0.32 | 0.76 | 0.43 | 0.66 | 4.66 | 0.2278 | 0.0756 | 0.1259 |
| 2544295 | 0.65 | 0.0044 | 1.56 | 0.64 | 0.4669 | 0.6 | 0.67 | 0.51 | 0.74 | 4.64 | 0.003 | 0.0075 | 0.0078 |
| 2544306 | 0.64 | 0.0078 | 1.36 | 0.63 | 0.611 | 0.49 | 0.71 | 0.49 | 0.71 | 5.25 | 0.0202 | 0.0174 | 0.0194 |
| 2546279 | 0.67 | 0.0019 | 1.58 | 0.67 | 0.2627 | 0.66 | 0.67 | 0.53 | 0.77 | 5.37 | 0.0003 | 0.0023 | 0.0021 |
| 2549323 | 0.62 | 0.0261 | 1.29 | 0.61 | 0.7405 | 0.53 | 0.66 | 0.47 | 0.71 | 4.71 | 0.0268 | 0.0109 | 0.0253 |
| 2550840 | 0.64 | 0.0107 | 1.25 | 0.66 | 0.3262 | 0.57 | 0.71 | 0.53 | 0.74 | 4.88 | 0.0006 | 0.0104 | 0.0193 |
| 2550842 | 0.63 | 0.012 | 1.34 | 0.6 | 0.8429 | 0.43 | 0.7 | 0.44 | 0.68 | 3.89 | 0.1429 | 0.0194 | 0.0269 |
| 2550964 | 0.7 | 0.0002 | 1.39 | 0.68 | 0.1571 | 0.62 | 0.72 | 0.56 | 0.77 | 5.94 | 0.0001 | 0.0002 | 0.0003 |
| 2552021 | 0.65 | 0.0057 | 1.53 | 0.65 | 0.3949 | 0.51 | 0.73 | 0.52 | 0.72 | 5.1 | 0.0037 | 0.0024 | 0.0035 |
| 2553312 | 0.65 | 0.0056 | 1.29 | 0.63 | 0.611 | 0.64 | 0.62 | 0.49 | 0.75 | 5.71 | 0.0039 | 0.0066 | 0.0065 |
| 2553583 | 0.67 | 0.0017 | 1.33 | 0.59 | 0.8822 | 0.74 | 0.5 | 0.46 | 0.77 | 7.07 | 0.009 | 0.002 | 0.0023 |
| 2553585 | 0.63 | 0.0128 | 1.75 | 0.66 | 0.3262 | 0.57 | 0.71 | 0.53 | 0.74 | 5.35 | 0.0009 | 0.0045 | 0.0077 |
| 2553595 | 0.66 | 0.0031 | 1.43 | 0.63 | 0.611 | 0.6 | 0.65 | 0.49 | 0.74 | 5.33 | 0.0044 | 0.0019 | 0.0033 |
| 2553907 | 0.66 | 0.0024 | 1.3 | 0.64 | 0.4669 | 0.45 | 0.76 | 0.51 | 0.7 | 4.47 | 0.008 | 0.002 | 0.0055 |
| 2554001 | 0.63 | 0.013 | 1.7 | 0.65 | 0.3949 | 0.53 | 0.72 | 0.52 | 0.73 | 3.88 | 0.0027 | 0.0089 | 0.0105 |
| 2555279 | 0.64 | 0.0084 | 1.42 | 0.62 | 0.6786 | 0.57 | 0.65 | 0.48 | 0.73 | 5.58 | 0.019 | 0.0103 | 0.0088 |
| 2555281 | 0.68 | 0.0005 | 1.51 | 0.66 | 0.3262 | 0.53 | 0.73 | 0.53 | 0.73 | 4.3 | 0.0012 | 0.0006 | 0.0011 |
| 2555288 | 0.65 | 0.0049 | 1.34 | 0.65 | 0.3949 | 0.6 | 0.68 | 0.52 | 0.75 | 4.22 | 0.0017 | 0.0059 | 0.0101 |
| 2555330 | 0.64 | 0.01 | 1.2 | 0.64 | 0.4669 | 0.38 | 0.79 | 0.51 | 0.69 | 4.29 | 0.0305 | 0.0048 | 0.0087 |
| 2555409 | 0.65 | 0.0056 | 1.39 | 0.68 | 0.1571 | 0.57 | 0.74 | 0.56 | 0.75 | 4.38 | 0.0001 | 0.0021 | 0.007 |
| 2555411 | 0.66 | 0.0022 | 1.51 | 0.63 | 0.611 | 0.68 | 0.6 | 0.49 | 0.77 | 5.34 | 0.0025 | 0.0013 | 0.0019 |
| 2555416 | 0.67 | 0.0014 | 1.36 | 0.66 | 0.3262 | 0.55 | 0.72 | 0.53 | 0.74 | 5.26 | 0.0015 | 0.0017 | 0.0034 |
| 2555507 | 0.63 | 0.0115 | 1.27 | 0.63 | 0.611 | 0.34 | 0.79 | 0.48 | 0.68 | 4.25 | 0.0667 | 0.0178 | 0.0274 |
| 2555522 | 0.65 | 0.0061 | 1.26 | 0.63 | 0.611 | 0.77 | 0.55 | 0.49 | 0.8 | 5.16 | 0.0011 | 0.0088 | 0.0087 |
| 2556229 | 0.65 | 0.0039 | 1.41 | 0.62 | 0.6786 | 0.55 | 0.66 | 0.48 | 0.72 | 4.17 | 0.0168 | 0.0085 | 0.0089 |
| 2558208 | 0.64 | 0.0092 | 1.28 | 0.67 | 0.2627 | 0.49 | 0.77 | 0.55 | 0.72 | 4.49 | 0.0011 | 0.0037 | 0.0087 |
| 2558313 | 0.64 | 0.0071 | 1.29 | 0.64 | 0.5397 | 0.64 | 0.63 | 0.5 | 0.75 | 5.23 | 0.0026 | 0.0029 | 0.0044 |
| 2558519 | 0.68 | 0.0005 | 1.39 | 0.68 | 0.1571 | 0.62 | 0.72 | 0.56 | 0.77 | 5.82 | 0.0001 | 0.0004 | 0.0007 |
| 2559293 | 0.61 | 0.0467 | 1.15 | 0.65 | 0.3949 | 0.47 | 0.76 | 0.52 | 0.71 | 5.3 | 0.0041 | 0.0244 | 0.0409 |
| 2562334 | 0.68 | 0.0007 | 1.33 | 0.66 | 0.3262 | 0.72 | 0.62 | 0.52 | 0.8 | 6.74 | 0.0002 | 0.0005 | 0.0008 |
| 2565264 | 0.67 | 0.0011 | 1.36 | 0.66 | 0.3262 | 0.72 | 0.62 | 0.52 | 0.8 | 7.58 | 0.0001 | 0.0008 | 0.0011 |
| 2575142 | 0.64 | 0.0084 | 1.49 | 0.65 | 0.3949 | 0.6 | 0.68 | 0.52 | 0.75 | 4.58 | 0.0017 | 0.0069 | 0.0074 |
| 2577959 | 0.65 | 0.006 | 1.32 | 0.67 | 0.206 | 0.47 | 0.79 | 0.56 | 0.72 | 4.14 | 0.0015 | 0.0085 | 0.0103 |
| 2581588 | 0.64 | 0.0106 | 1.32 | 0.58 | 0.914 | 0.55 | 0.6 | 0.44 | 0.7 | 4.27 | 0.0751 | 0.0171 | 0.0237 |
| 2583054 | 0.66 | 0.0032 | 1.33 | 0.64 | 0.5397 | 0.7 | 0.6 | 0.5 | 0.78 | 6.28 | 0.0012 | 0.0021 | 0.0029 |
| 2583056 | 0.67 | 0.0016 | 1.44 | 0.69 | 0.1164 | 0.7 | 0.68 | 0.56 | 0.8 | 4.41 | 0 | 0.0026 | 0.0027 |
| 2583249 | 0.63 | 0.0164 | 1.45 | 0.64 | 0.5397 | 0.55 | 0.68 | 0.5 | 0.73 | 4.69 | 0.0068 | 0.0125 | 0.0171 |
| 2583609 | 0.61 | 0.0382 | 1.33 | 0.6 | 0.7956 | 0.6 | 0.61 | 0.47 | 0.72 | 5.26 | 0.0229 | 0.0228 | 0.0302 |
| 2584845 | 0.68 | 0.0007 | 1.41 | 0.65 | 0.3949 | 0.66 | 0.65 | 0.52 | 0.77 | 6.12 | 0.0005 | 0.0006 | 0.0012 |
| 2588838 | 0.65 | 0.0058 | 1.3 | 0.67 | 0.2627 | 0.55 | 0.73 | 0.54 | 0.74 | 5.31 | 0.0003 | 0.0105 | 0.0173 |
| 2590750 | 0.64 | 0.0093 | 1.46 | 0.67 | 0.2627 | 0.62 | 0.7 | 0.54 | 0.76 | 5.42 | 0.0002 | 0.0062 | 0.0111 |
| 2590802 | 0.65 | 0.0055 | 1.6 | 0.67 | 0.2627 | 0.62 | 0.7 | 0.54 | 0.76 | 6.66 | 0.0002 | 0.002 | 0.0036 |
| 2591635 | 0.61 | 0.0472 | 1.31 | 0.6 | 0.8429 | 0.66 | 0.56 | 0.46 | 0.74 | 6.53 | 0.0108 | 0.0229 | 0.0396 |
| 2591638 | 0.64 | 0.0097 | 1.44 | 0.66 | 0.3262 | 0.64 | 0.67 | 0.53 | 0.76 | 6.56 | 0.0002 | 0.0034 | 0.0094 |
| 2593447 | 0.65 | 0.0044 | 1.37 | 0.63 | 0.611 | 0.55 | 0.67 | 0.49 | 0.72 | 4.58 | 0.0108 | 0.0066 | 0.0118 |
| 2593692 | 0.65 | 0.0052 | 1.56 | 0.62 | 0.6786 | 0.57 | 0.65 | 0.48 | 0.73 | 5.33 | 0.012 | 0.0033 | 0.0041 |
| 2593714 | 0.62 | 0.0236 | 1.31 | 0.61 | 0.7405 | 0.47 | 0.7 | 0.47 | 0.7 | 5.08 | 0.0502 | 0.0143 | 0.0259 |
| 2593758 | 0.61 | 0.0323 | 1.63 | 0.62 | 0.6786 | 0.66 | 0.6 | 0.48 | 0.75 | 4.96 | 0.0031 | 0.0086 | 0.0156 |
| 2594506 | 0.65 | 0.006 | 1.4 | 0.64 | 0.5397 | 0.57 | 0.67 | 0.5 | 0.73 | 4.18 | 0.007 | 0.0149 | 0.0127 |
| 2596691 | 0.65 | 0.0043 | 1.3 | 0.63 | 0.611 | 0.72 | 0.57 | 0.49 | 0.78 | 4.81 | 0.0017 | 0.007 | 0.0057 |
| 2597013 | 0.65 | 0.0043 | 1.32 | 0.59 | 0.8822 | 0.64 | 0.56 | 0.45 | 0.73 | 4.47 | 0.0208 | 0.0019 | 0.0039 |
| 2598267 | 0.64 | 0.0084 | 1.35 | 0.64 | 0.5397 | 0.68 | 0.61 | 0.5 | 0.77 | 6.26 | 0.0015 | 0.0032 | 0.0099 |
| 2598289 | 0.67 | 0.0014 | 1.46 | 0.67 | 0.2627 | 0.6 | 0.71 | 0.54 | 0.75 | 5.61 | 0.001 | 0.0008 | 0.0029 |
| 2598781 | 0.66 | 0.0031 | 1.3 | 0.64 | 0.4669 | 0.72 | 0.6 | 0.51 | 0.79 | 8.69 | 0.0005 | 0.0021 | 0.0028 |
| 2599342 | 0.66 | 0.0024 | 1.5 | 0.66 | 0.3262 | 0.57 | 0.71 | 0.53 | 0.74 | 5.64 | 0.0018 | 0.0033 | 0.0041 |
| 2599903 | 0.63 | 0.0155 | 1.47 | 0.63 | 0.611 | 0.57 | 0.66 | 0.49 | 0.73 | 4.36 | 0.0141 | 0.0094 | 0.0095 |
| 2606647 | 0.63 | 0.0151 | 1.36 | 0.6 | 0.8429 | 0.66 | 0.56 | 0.46 | 0.74 | 6.61 | 0.0171 | 0.0096 | 0.0108 |
| 2607152 | 0.64 | 0.0066 | 1.31 | 0.65 | 0.3949 | 0.66 | 0.65 | 0.52 | 0.77 | 6.02 | 0.0008 | 0.0046 | 0.0075 |
| 2607374 | 0.64 | 0.0086 | 1.37 | 0.62 | 0.6786 | 0.45 | 0.72 | 0.48 | 0.69 | 5.08 | 0.0487 | 0.0072 | 0.0108 |
| 2609635 | 0.65 | 0.0051 | 1.34 | 0.64 | 0.5397 | 0.68 | 0.61 | 0.5 | 0.77 | 4.55 | 0.0012 | 0.0052 | 0.0063 |
| 2609657 | 0.68 | 0.001 | 1.41 | 0.69 | 0.1164 | 0.64 | 0.72 | 0.57 | 0.78 | 5.09 | 0.0001 | 0.0006 | 0.0013 |
| 2609668 | 0.64 | 0.0074 | 1.29 | 0.63 | 0.611 | 0.68 | 0.6 | 0.49 | 0.77 | 5.61 | 0.0015 | 0.0077 | 0.0179 |
| 2610353 | 0.63 | 0.0177 | 1.37 | 0.6 | 0.8429 | 0.66 | 0.56 | 0.46 | 0.74 | 5.45 | 0.012 | 0.0113 | 0.0179 |
| 2611083 | 0.64 | 0.0093 | 1.27 | 0.64 | 0.4669 | 0.38 | 0.79 | 0.51 | 0.69 | 5.77 | 0.0163 | 0.0037 | 0.0079 |
| 2614100 | 0.65 | 0.0041 | 1.36 | 0.67 | 0.206 | 0.57 | 0.73 | 0.55 | 0.75 | 5.7 | 0.0003 | 0.0075 | 0.0056 |
| 2614135 | 0.64 | 0.0101 | 1.27 | 0.61 | 0.7405 | 0.51 | 0.67 | 0.47 | 0.71 | 5.24 | 0.0455 | 0.0078 | 0.0121 |
| 2614137 | 0.63 | 0.0118 | 1.44 | 0.65 | 0.3949 | 0.64 | 0.66 | 0.52 | 0.76 | 5.64 | 0.0012 | 0.0057 | 0.0055 |
| 2615656 | 0.62 | 0.03 | 1.25 | 0.58 | 0.914 | 0.51 | 0.62 | 0.44 | 0.69 | 5.34 | 0.1141 | 0.0366 | 0.0484 |
| 2616071 | 0.62 | 0.0194 | 1.47 | 0.64 | 0.4669 | 0.51 | 0.72 | 0.51 | 0.72 | 4.56 | 0.0043 | 0.0077 | 0.0167 |
| 2616248 | 0.65 | 0.0055 | 1.36 | 0.64 | 0.5397 | 0.53 | 0.7 | 0.5 | 0.72 | 5.09 | 0.0112 | 0.0054 | 0.0055 |
| 2617578 | 0.64 | 0.007 | 1.41 | 0.58 | 0.914 | 0.74 | 0.49 | 0.45 | 0.77 | 5.27 | 0.0116 | 0.0066 | 0.0071 |
| 2618656 | 0.67 | 0.0013 | 1.49 | 0.61 | 0.7405 | 0.7 | 0.56 | 0.48 | 0.77 | 5.76 | 0.0049 | 0.0015 | 0.0014 |
| 2619365 | 0.64 | 0.0078 | 1.38 | 0.64 | 0.4669 | 0.57 | 0.68 | 0.51 | 0.74 | 4.8 | 0.003 | 0.011 | 0.0141 |
| 2625632 | 0.62 | 0.0242 | 1.2 | 0.58 | 0.914 | 0.66 | 0.54 | 0.45 | 0.73 | 3.76 | 0.0335 | 0.0467 | 0.0408 |
| 2625643 | 0.7 | 0.0002 | 1.35 | 0.66 | 0.3262 | 0.53 | 0.73 | 0.53 | 0.73 | 6.21 | 0.0026 | 0.0005 | 0.0006 |
| 2625893 | 0.67 | 0.0017 | 1.41 | 0.63 | 0.611 | 0.45 | 0.73 | 0.49 | 0.7 | 3.81 | 0.0318 | 0.0037 | 0.0042 |
| 2627951 | 0.7 | 0.0002 | 1.37 | 0.69 | 0.1164 | 0.66 | 0.71 | 0.56 | 0.78 | 3.94 | 0 | 0.0005 | 0.0009 |
| 2633534 | 0.67 | 0.0015 | 1.3 | 0.63 | 0.611 | 0.45 | 0.73 | 0.49 | 0.7 | 4.58 | 0.0355 | 0.0017 | 0.0024 |
| 2633651 | 0.64 | 0.0069 | 1.4 | 0.66 | 0.3262 | 0.62 | 0.68 | 0.53 | 0.76 | 5.81 | 0.0004 | 0.0033 | 0.0053 |
| 2638377 | 0.65 | 0.0046 | 1.28 | 0.62 | 0.6786 | 0.49 | 0.7 | 0.48 | 0.7 | 4.39 | 0.0182 | 0.0047 | 0.0075 |
| 2638451 | 0.64 | 0.073 | 1.29 | 0.64 | 0.5397 | 0.64 | 0.63 | 0.5 | 0.75 | 7.12 | 0.0018 | 0.0036 | 0.0057 |
| 2638452 | 0.64 | 0.0101 | 1.37 | 0.6 | 0.7956 | 0.72 | 0.54 | 0.47 | 0.77 | 6.85 | 0.0049 | 0.0069 | 0.0076 |
| 2641575 | 0.63 | 0.011 | 1.34 | 0.62 | 0.6786 | 0.57 | 0.65 | 0.48 | 0.73 | 5.27 | 0.0126 | 0.0137 | 0.0133 |
| 2643579 | 0.62 | 0.0194 | 1.29 | 0.65 | 0.3949 | 0.55 | 0.71 | 0.52 | 0.73 | 4.93 | 0.001 | 0.0136 | 0.0179 |
| 2645294 | 0.67 | 0.0017 | 1.63 | 0.68 | 0.1571 | 0.6 | 0.73 | 0.56 | 0.76 | 5.95 | 0.0001 | 0.0006 | 0.0011 |
| 2646066 | 0.64 | 0.01 | 1.3 | 0.66 | 0.3262 | 0.7 | 0.63 | 0.52 | 0.79 | 5.12 | 0.0003 | 0.0124 | 0.0148 |
| 2646085 | 0.66 | 0.0026 | 1.54 | 0.62 | 0.6786 | 0.7 | 0.57 | 0.49 | 0.77 | 5.23 | 0.0029 | 0.0013 | 0.0022 |
| 2647361 | 0.64 | 0.007 | 1.46 | 0.66 | 0.3262 | 0.62 | 0.68 | 0.53 | 0.76 | 5.11 | 0.0005 | 0.0077 | 0.0102 |
| 2647678 | 0.65 | 0.0044 | 1.31 | 0.67 | 0.2627 | 0.55 | 0.73 | 0.54 | 0.74 | 4.46 | 0.0004 | 0.0031 | 0.0062 |
| 2647775 | 0.66 | 0.0028 | 1.39 | 0.66 | 0.3262 | 0.57 | 0.71 | 0.53 | 0.74 | 5.37 | 0.0006 | 0.0014 | 0.003 |
| 2647779 | 0.64 | 0.0107 | 1.32 | 0.6 | 0.7956 | 0.57 | 0.62 | 0.47 | 0.72 | 4.28 | 0.0257 | 0.0028 | 0.0081 |
| 2648202 | 0.62 | 0.0213 | 1.25 | 0.65 | 0.3949 | 0.47 | 0.76 | 0.52 | 0.71 | 5.46 | 0.0116 | 0.0259 | 0.0292 |
| 2651521 | 0.68 | 0.0006 | 1.35 | 0.7 | 0.0837 | 0.64 | 0.73 | 0.58 | 0.78 | 4.48 | 0 | 0.0003 | 0.0011 |
| 2652021 | 0.67 | 0.0016 | 1.51 | 0.67 | 0.2627 | 0.57 | 0.72 | 0.54 | 0.75 | 5.14 | 0.0005 | 0.001 | 0.0023 |
| 2654335 | 0.68 | 0.0007 | 1.38 | 0.64 | 0.4669 | 0.47 | 0.74 | 0.51 | 0.71 | 5.6 | 0.0075 | 0.0009 | 1.0011 |
| 2654874 | 0.67 | 0.001 | 1.34 | 0.68 | 0.1571 | 0.64 | 0.71 | 0.56 | 0.77 | 5.87 | 0.0001 | 0.0011 | 0.0014 |
| 2654878 | 0.67 | 0.0017 | 1.29 | 0.66 | 0.3262 | 0.45 | 0.78 | 0.54 | 0.71 | 4.18 | 0.0021 | 0.0006 | 0.0026 |
| 2655475 | 0.68 | 0.0008 | 1.39 | 0.67 | 0.206 | 0.57 | 0.73 | 0.55 | 0.75 | 5.38 | 0.0002 | 0.0007 | 0.0011 |
| 2655509 | 0.66 | 0.0023 | 1.3 | 0.63 | 0.611 | 0.7 | 0.59 | 0.49 | 0.77 | 8.77 | 0.0016 | 0.0012 | 0.0021 |
| 2655678 | 0.64 | 0.0097 | 1.31 | 0.62 | 0.6786 | 0.62 | 0.62 | 0.48 | 0.74 | 5.74 | 0.0036 | 0.004 | 0.01 |
| 2656791 | 0.66 | 0.0024 | 1.31 | 0.62 | 0.6786 | 0.72 | 0.56 | 0.49 | 0.78 | 6.96 | 0.0024 | 0.0025 | 0.0033 |
| 2658354 | 0.66 | 0.0032 | 1.23 | 0.67 | 0.2627 | 0.6 | 0.71 | 0.54 | 0.75 | 5.34 | 0.001 | 0.0033 | 0.0037 |
| 2658632 | 0.72 | 0 | 1.39 | 0.69 | 0.1164 | 0.64 | 0.72 | 0.57 | 0.78 | 5.01 | 0 | 0 | 0.0001 |
| 2663553 | 0.61 | 0.043 | 1.32 | 0.64 | 0.4669 | 0.47 | 0.74 | 0.51 | 0.71 | 4.81 | 0.0087 | 0.011 | 0.0206 |
| 2666503 | 0.61 | 0.0456 | 1.39 | 0.67 | 0.2627 | 0.57 | 0.72 | 0.54 | 0.75 | 6.15 | 0.0005 | 0.0167 | 0.027 |
| 2666519 | 0.64 | 0.0096 | 1.2 | 0.64 | 0.4669 | 0.45 | 0.76 | 0.51 | 0.7 | 6.7 | 0.0174 | 0.0067 | 0.0104 |
| 2666524 | 0.66 | 0.0031 | 1.47 | 0.63 | 0.611 | 0.53 | 0.68 | 0.49 | 0.72 | 4.57 | 0.0072 | 0.0013 | 0.0035 |
| 2669316 | 0.62 | 0.0271 | 1.55 | 0.64 | 0.5397 | 0.53 | 0.7 | 0.5 | 0.72 | 4.86 | 0.0115 | 0.0177 | 0.0154 |
| 2669571 | 0.67 | 0.0014 | 1.41 | 0.66 | 0.3262 | 0.64 | 0.67 | 0.53 | 0.76 | 5.11 | 0.001 | 0.0034 | 0.0029 |
| 2670438 | 0.68 | 0.0008 | 0.71 | 0.36 | 1 | 0.36 | 0.37 | 0.25 | 0.5 | 4.35 | 0.0086 | 0.0035 | 0.0013 |
| 2672775 | 0.63 | 0.0121 | 1.5 | 0.59 | 0.8822 | 0.6 | 0.59 | 0.45 | 0.72 | 4.97 | 0.0351 | 0.007 | 0.0121 |
| 2674243 | 0.63 | 0.0113 | 1.4 | 0.6 | 0.7956 | 0.7 | 0.55 | 0.47 | 0.76 | 5.96 | 0.0056 | 0.011 | 0.0127 |
| 2675269 | 0.61 | 0.0339 | 1.2 | 0.6 | 0.8429 | 0.45 | 0.68 | 0.45 | 0.68 | 4.13 | 0.1106 | 0.0587 | 0.0831 |
| 2675802 | 0.61 | 0.0315 | 1.34 | 0.63 | 0.611 | 0.55 | 0.67 | 0.49 | 0.72 | 5.9 | 0.0157 | 0.0202 | 0.0209 |
| 2676931 | 0.65 | 0.0051 | 1.37 | 0.64 | 0.5397 | 0.62 | 0.65 | 0.5 | 0.75 | 6.46 | 0.0021 | 0.0049 | 0.0057 |
| 2677655 | 0.63 | 0.0141 | 1.33 | 0.64 | 0.4669 | 0.64 | 0.65 | 0.51 | 0.76 | 5.91 | 0.002 | 0.0134 | 0.0126 |
| 2677695 | 0.69 | 0.0004 | 1.32 | 0.64 | 0.5397 | 0.49 | 0.72 | 0.5 | 0.71 | 4.41 | 0.0088 | 0.0003 | 0.0009 |
| 2680511 | 0.65 | 0.0036 | 1.33 | 0.69 | 0.1164 | 0.64 | 0.72 | 0.57 | 0.78 | 3.39 | 0 | 0.0021 | 0.0034 |
| 2682663 | 0.66 | 0.0024 | 1.34 | 0.68 | 0.1571 | 0.53 | 0.77 | 0.57 | 0.74 | 6.54 | 0.0011 | 0.0023 | 0.0036 |
| 2685954 | 0.62 | 0.0252 | 1.26 | 0.61 | 0.7405 | 0.38 | 0.74 | 0.46 | 0.68 | 3.8 | 0.072 | 0.0329 | 0.0816 |
| 2691756 | 0.67 | 0.001 | 1.31 | 0.64 | 0.4669 | 0.64 | 0.65 | 0.51 | 0.76 | 4.83 | 0.0011 | 0.0014 | 0.0018 |
| 2692111 | 0.67 | 0.0012 | 1.57 | 0.69 | 0.1164 | 0.66 | 0.71 | 0.56 | 0.78 | 5.09 | 0.0001 | 0.0022 | 0.0018 |
| 2694407 | 0.69 | 0.0005 | 1.32 | 0.64 | 0.5397 | 0.6 | 0.66 | 0.5 | 0.74 | 3.99 | 0.0032 | 0.0006 | 0.0021 |
| 2694648 | 0.64 | 0.0101 | 1.44 | 0.6 | 0.8429 | 0.62 | 0.59 | 0.46 | 0.73 | 5.7 | 0.024 | 0.0089 | 0.0113 |
| 2694649 | 0.65 | 0.004 | 1.45 | 0.61 | 0.7405 | 0.64 | 0.6 | 0.48 | 0.74 | 5.44 | 0.0085 | 0.0042 | 0.0064 |
| 2695413 | 0.66 | 0.0031 | 1.39 | 0.66 | 0.3262 | 0.53 | 0.73 | 0.53 | 0.73 | 6.08 | 0.001 | 0.0014 | 0.0026 |
| 2700507 | 0.63 | 0.0159 | 1.27 | 0.6 | 0.7956 | 0.43 | 0.71 | 0.45 | 0.68 | 5.32 | 0.0742 | 0.0166 | 0.0338 |
| 2700829 | 0.66 | 0.0023 | 1.39 | 0.61 | 0.7405 | 0.47 | 0.7 | 0.47 | 0.7 | 5.51 | 0.0397 | 0.003 | 0.0055 |
| 2701289 | 0.65 | 0.0045 | 1.31 | 0.65 | 0.3949 | 0.64 | 0.66 | 0.52 | 0.76 | 5.43 | 0.0009 | 0.0062 | 0.0058 |
| 2702329 | 0.61 | 0.0303 | 1.33 | 0.63 | 0.611 | 0.6 | 0.65 | 0.49 | 0.74 | 4.59 | 0.0064 | 0.0177 | 0.0298 |
| 2703240 | 0.65 | 0.0054 | 1.52 | 0.64 | 0.4669 | 0.47 | 0.74 | 0.51 | 0.71 | 4.59 | 0.0064 | 0.0037 | 0.0075 |
| 2704511 | 0.66 | 0.0023 | 1.23 | 0.67 | 0.206 | 0.55 | 0.74 | 0.55 | 0.74 | 3.46 | 0.0008 | 0.0006 | 0.0009 |
| 2704898 | 0.66 | 0.0026 | 1.37 | 0.62 | 0.6786 | 0.51 | 0.68 | 0.48 | 0.71 | 4.15 | 0.0193 | 0.0013 | 0.0029 |
| 2704935 | 0.64 | 0.0094 | 1.29 | 0.65 | 0.3949 | 0.66 | 0.65 | 0.52 | 0.77 | 7.15 | 0.0005 | 0.0039 | 0.0085 |
| 2705001 | 0.69 | 0.0004 | 1.54 | 0.67 | 0.206 | 0.64 | 0.7 | 0.55 | 0.77 | 6.2 | 0.0001 | 0.0001 | 0.0003 |
| 2706322 | 0.65 | 0.0049 | 1.36 | 0.66 | 0.3262 | 0.55 | 0.72 | 0.53 | 0.74 | 5.24 | 0.0006 | 0.0025 | 0.005 |
| 2706324 | 0.63 | 0.0132 | 1.36 | 0.66 | 0.3262 | 0.64 | 0.67 | 0.53 | 0.76 | 6.14 | 0.0005 | 0.0064 | 0.0107 |
| 2707780 | 0.66 | 0.0031 | 1.38 | 0.63 | 0.611 | 0.57 | 0.66 | 0.49 | 0.73 | 5.34 | 0.0092 | 0.0022 | 0.0048 |
| 2708798 | 0.67 | 0.0011 | 1.36 | 0.64 | 0.4669 | 0.7 | 0.61 | 0.51 | 0.78 | 7.58 | 0.0007 | 0.0012 | 0.0017 |
| 2709489 | 0.61 | 0.0382 | 1.25 | 0.64 | 0.5397 | 0.53 | 0.7 | 0.5 | 0.72 | 5.65 | 0.0044 | 0.0378 | 0.0499 |
| 2710214 | 0.65 | 0.0041 | 1.31 | 0.62 | 0.6786 | 0.45 | 0.72 | 0.48 | 0.69 | 4.56 | 0.0535 | 0.0046 | 0.0057 |
| 2710217 | 0.71 | 0.0001 | 1.4 | 0.69 | 0.1164 | 0.55 | 0.77 | 0.58 | 0.75 | 4.85 | 0.0002 | 0.0002 | 0.0003 |
| 2711689 | 0.63 | 0.0173 | 1.32 | 0.61 | 0.7405 | 0.57 | 0.63 | 0.47 | 0.72 | 4.1 | 0.0134 | 0.0191 | 0.0379 |
| 2712424 | 0.65 | 0.0052 | 1.44 | 0.64 | 0.4669 | 0.68 | 0.62 | 0.51 | 0.77 | 6.13 | 0.0013 | 0.0031 | 0.0037 |
| 2713075 | 0.64 | 0.0104 | 1.46 | 0.6 | 0.8429 | 0.6 | 0.6 | 0.46 | 0.72 | 6.49 | 0.0219 | 0.0063 | 0.0084 |
| 2713262 | 0.66 | 0.0024 | 1.34 | 0.67 | 0.206 | 0.62 | 0.71 | 0.55 | 0.76 | 4.38 | 0.0001 | 0.0037 | 0.001 |
| 2713269 | 0.68 | 0.0005 | 1.35 | 0.64 | 0.4669 | 0.55 | 0.7 | 0.51 | 0.73 | 5.43 | 0.0032 | 0.0005 | 0.0009 |
| 2714106 | 0.64 | 0.007 | 1.29 | 0.58 | 0.914 | 0.34 | 0.72 | 0.41 | 0.66 | 4.95 | 0.4954 | 0.02 | 0.0209 |
| 2715470 | 0.63 | 0.0173 | 1.41 | 0.59 | 0.8822 | 0.72 | 0.51 | 0.46 | 0.76 | 5.36 | 0.0088 | 0.0119 | 0.0146 |
| 2715717 | 0.65 | 0.0038 | 1.77 | 0.6 | 0.7956 | 0.68 | 0.56 | 0.47 | 0.75 | 5.55 | 0.0062 | 0.0036 | 0.0045 |
| 2720693 | 0.66 | 0.0031 | 1.38 | 0.64 | 0.4669 | 0.47 | 0.74 | 0.51 | 0.71 | 4.42 | 0.0093 | 0.0129 | 0.0129 |
| 2724050 | 0.67 | 0.001 | 1.36 | 0.66 | 0.3262 | 0.57 | 0.71 | 0.53 | 0.74 | 5.65 | 0.0004 | 0.0004 | 0.0013 |
| 2724565 | 0.67 | 0.0017 | 1.35 | 0.64 | 0.5397 | 0.7 | 0.6 | 0.5 | 0.8 | 8.11 | 0.0011 | 0.0004 | 0.001 |
| 2726514 | 0.65 | 0.0051 | 1.34 | 0.62 | 0.6786 | 0.53 | 0.67 | 0.48 | 0.71 | 4.49 | 0.0198 | 0.0088 | 0.01 |
| 2727970 | 0.64 | 0.0101 | 1.48 | 0.63 | 0.611 | 0.64 | 0.62 | 0.49 | 0.75 | 5.17 | 0.004 | 0.0077 | 0.0084 |
| 2730701 | 0.64 | 0.0096 | 1.31 | 0.6 | 0.7956 | 0.45 | 0.7 | 0.46 | 0.69 | 4.41 | 0.0935 | 0.0163 | 0.0202 |
| 2731872 | 0.67 | 0.0015 | 1.49 | 0.65 | 0.3949 | 0.45 | 0.77 | 0.53 | 0.71 | 4.78 | 0.0066 | 0.0033 | 0.0041 |
| 2732403 | 0.63 | 0.0115 | 1.38 | 0.62 | 0.6786 | 0.64 | 0.61 | 0.48 | 0.75 | 4.77 | 0.0058 | 0.0054 | 0.0135 |
| 2735017 | 0.63 | 0.0118 | 1.36 | 0.63 | 0.611 | 0.43 | 0.74 | 0.49 | 0.69 | 4.91 | 0.0495 | 0.0377 | 0.0262 |
| 2736295 | 0.63 | 0.0153 | 1.41 | 0.64 | 0.5397 | 0.53 | 0.7 | 0.5 | 0.72 | 4.58 | 0.0078 | 0.011 | 0.0193 |
| 2738963 | 0.65 | 0.0052 | 1.4 | 0.64 | 0.4669 | 0.62 | 0.66 | 0.51 | 0.75 | 5.13 | 0.0033 | 0.0048 | 0.0064 |
| 2743194 | 0.68 | 0.0006 | 1.3 | 0.64 | 0.4669 | 0.68 | 0.62 | 0.51 | 0.77 | 6.67 | 0.0008 | 0.0001 | 0.0005 |
| 2744632 | 0.58 | 0.1112 | 1.25 | 0.62 | 0.6786 | 0.47 | 0.71 | 0.48 | 0.7 | 4.28 | 0.042 | 0.1095 | 0.147 |
| 2745687 | 0.65 | 0.0041 | 1.32 | 0.64 | 0.5397 | 0.57 | 0.67 | 0.5 | 0.73 | 5.03 | 0.0056 | 0.0086 | 0.0125 |
| 2754300 | 0.65 | 0.0045 | 1.29 | 0.64 | 0.4669 | 0.47 | 0.74 | 0.51 | 0.71 | 4.77 | 0.0171 | 0.0106 | 0.0088 |
| 2757037 | 0.66 | 0.003 | 1.35 | 0.61 | 0.7405 | 0.74 | 0.54 | 0.48 | 0.79 | 7.13 | 0.0017 | 0.005 | 0.0068 |
| 2763813 | 0.62 | 0.0282 | 1.34 | 0.61 | 0.7405 | 0.47 | 0.7 | 0.47 | 0.7 | 3.84 | 0.0581 | 0.0175 | 0.0303 |
| 2766634 | 0.66 | 0.0019 | 1.33 | 0.65 | 0.3949 | 0.7 | 0.62 | 0.52 | 0.78 | 4.74 | 0.0006 | 0.0022 | 0.0042 |
| 2770195 | 0.63 | 0.0121 | 1.29 | 0.64 | 0.5397 | 0.4 | 0.77 | 0.5 | 0.69 | 3.7 | 0.0313 | 0.0114 | 0.0156 |
| 2770473 | 0.66 | 0.0025 | 1.4 | 0.66 | 0.3262 | 0.64 | 0.67 | 0.53 | 0.76 | 6.63 | 0.0006 | 0.002 | 0.0023 |
| 2771759 | 0.68 | 0.0006 | 1.43 | 0.69 | 0.1164 | 0.55 | 0.77 | 0.58 | 0.75 | 5.36 | 0.0002 | 0.0003 | 0.0004 |
| 2773129 | 0.62 | 0.0221 | 1.32 | 0.59 | 0.8822 | 0.64 | 0.56 | 0.45 | 0.73 | 4.12 | 0.0343 | 0.0262 | 0.0211 |
| 2774054 | 0.64 | 0.0078 | 1.38 | 0.64 | 0.5397 | 0.43 | 0.76 | 0.5 | 0.7 | 4.42 | 0.037 | 0.0161 | 0.0109 |
| 2775592 | 0.6 | 0.0547 | 1.23 | 0.58 | 0.914 | 0.6 | 0.57 | 0.44 | 0.71 | 4.96 | 0.0661 | 0.023 | 0.0372 |
| 2775758 | 0.65 | 0.0055 | 1.41 | 0.61 | 0.7405 | 0.72 | 0.55 | 0.48 | 0.78 | 6.87 | 0.0025 | 0.0071 | 0.0101 |
| 2776094 | 0.64 | 0.0077 | 1.38 | 0.66 | 0.3262 | 0.62 | 0.68 | 0.53 | 0.76 | 4.13 | 0.0016 | 0.0434 | 0.0197 |
| 2777492 | 0.61 | 0.0327 | 1.31 | 0.64 | 0.5397 | 0.43 | 0.76 | 0.5 | 0.7 | 4 | 0.0178 | 0.009 | 0.0225 |
| 2779488 | 0.67 | 0.0011 | 1.39 | 0.67 | 0.206 | 0.64 | 0.7 | 0.55 | 0.77 | 6.72 | 0.0001 | 0.0004 | 0.001 |
| 2779639 | 0.66 | 0.0029 | 1.48 | 0.62 | 0.6786 | 0.64 | 0.61 | 0.48 | 0.75 | 5.71 | 0.0043 | 0.0028 | 0.003 |
| 2779995 | 0.63 | 0.0149 | 1.34 | 0.63 | 0.611 | 0.55 | 0.67 | 0.49 | 0.72 | 5.44 | 0.0121 | 0.0076 | 0.0095 |
| 2780545 | 0.67 | 0.0011 | 1.44 | 0.67 | 0.206 | 0.62 | 0.71 | 0.55 | 0.76 | 4.57 | 0.0002 | 0.0012 | 0.0022 |
| 2783475 | 0.67 | 0.0016 | 1.39 | 0.64 | 0.4669 | 0.72 | 0.6 | 0.51 | 0.79 | 6.7 | 0.0005 | 0.0013 | 0.0015 |
| 2789554 | 0.62 | 0.0227 | 1.17 | 0.61 | 0.7405 | 0.49 | 0.68 | 0.47 | 0.7 | 5.7 | 0.0366 | 0.0162 | 0.0293 |
| 2790573 | 0.63 | 0.0128 | 1.32 | 0.63 | 0.611 | 0.74 | 0.56 | 0.49 | 0.79 | 6.09 | 0.0012 | 0.0137 | 0.0111 |
| 2797449 | 0.65 | 0.0042 | 1.33 | 0.63 | 0.611 | 0.53 | 0.68 | 0.49 | 0.72 | 6.3 | 0.0131 | 0.005 | 0.0068 |
| 2801338 | 0.62 | 0.02 | 1.47 | 0.64 | 0.5397 | 0.51 | 0.71 | 0.5 | 0.72 | 4.65 | 0.0066 | 0.0168 | 0.0265 |
| 2801556 | 0.65 | 0.0044 | 1.34 | 0.6 | 0.7956 | 0.68 | 0.56 | 0.47 | 0.75 | 6.7 | 0.0063 | 0.0023 | 0.0036 |
| 2801679 | 0.66 | 0.0029 | 1.33 | 0.64 | 0.5397 | 0.66 | 0.62 | 0.5 | 0.76 | 6.36 | 0.002 | 0.0013 | 0.0026 |
| 2802506 | 0.69 | 0.0003 | 1.33 | 0.64 | 0.4669 | 0.74 | 0.59 | 0.51 | 0.8 | 4.36 | 0.0002 | 0.0003 | 0.0013 |
| 2805739 | 0.61 | 0.0356 | 1.21 | 0.6 | 0.7956 | 0.47 | 0.68 | 0.46 | 0.69 | 4.4 | 0.0679 | 0.0264 | 0.0409 |
| 2806859 | 0.68 | 0.0007 | 1.29 | 0.67 | 0.2627 | 0.64 | 0.68 | 0.54 | 0.77 | 6.08 | 0.0003 | 0.0003 | 0.0006 |
| 2806912 | 0.64 | 0.0109 | 1.34 | 0.63 | 0.611 | 0.57 | 0.66 | 0.49 | 0.73 | 5.86 | 0.0079 | 0.0078 | 0.012 |
| 2807658 | 0.69 | 0.0004 | 1.34 | 0.67 | 0.2627 | 0.57 | 0.72 | 0.54 | 0.75 | 5.22 | 0.0003 | 0.0002 | 0.0006 |
| 2809146 | 0.61 | 0.0303 | 1.43 | 0.62 | 0.6786 | 0.64 | 0.61 | 0.48 | 0.75 | 5.49 | 0.0056 | 0.0171 | 0.0207 |
| 2810487 | 0.61 | 0.0477 | 1.25 | 0.61 | 0.7405 | 0.47 | 0.7 | 0.47 | 0.7 | 4.2 | 0.0389 | 0.0321 | 0.0581 |
| 2812307 | 0.65 | 0.0046 | 1.46 | 0.64 | 0.5397 | 0.53 | 0.7 | 0.5 | 0.72 | 4.52 | 0.0133 | 0.015 | 0.0115 |
| 2812500 | 0.64 | 0.0078 | 1.6 | 0.64 | 0.4669 | 0.53 | 0.71 | 0.51 | 0.73 | 3.58 | 0.0058 | 0.0023 | 0.0051 |
| 2812573 | 0.64 | 0.0092 | 1.28 | 0.62 | 0.6786 | 0.68 | 0.59 | 0.48 | 0.76 | 7 | 0.0034 | 0.0044 | 0.0073 |
| 2817404 | 0.67 | 0.0019 | 1.66 | 0.67 | 0.206 | 0.57 | 0.73 | 0.55 | 0.75 | 4.65 | 0.0007 | 0.0042 | 0.0046 |
| 2817457 | 0.66 | 0.0031 | 1.66 | 0.64 | 0.5397 | 0.57 | 0.67 | 0.5 | 0.73 | 4.85 | 0.0067 | 0.0034 | 0.0035 |
| 2818271 | 0.63 | 0.0118 | 1.25 | 0.67 | 0.206 | 0.64 | 0.7 | 0.55 | 0.77 | 6.67 | 0.0001 | 0.0085 | 0.017 |
| 2818964 | 0.67 | 0.0014 | 1.4 | 0.63 | 0.611 | 0.72 | 0.57 | 0.49 | 0.78 | 6.77 | 0.0011 | 0.0018 | 0.002 |
| 2822274 | 0.61 | 0.0315 | 1.7 | 0.64 | 0.5397 | 0.6 | 0.66 | 0.5 | 0.74 | 5.01 | 0.0025 | 0.0132 | 0.0191 |
| 2823066 | 0.68 | 0.001 | 1.36 | 0.67 | 0.2627 | 0.43 | 0.8 | 0.56 | 0.71 | 4.03 | 0.003 | 0.0041 | 0.0047 |
| 2823854 | 0.63 | 0.0135 | 1.44 | 0.59 | 0.8822 | 0.64 | 0.56 | 0.45 | 0.73 | 6.26 | 0.0217 | 0.0078 | 0.0118 |
| 2824899 | 0.62 | 0.0265 | 1.26 | 0.63 | 0.611 | 0.49 | 0.71 | 0.49 | 0.71 | 4.63 | 0.0104 | 0.0181 | 0.0374 |
| 2828112 | 0.67 | 0.0015 | 1.61 | 0.66 | 0.3262 | 0.72 | 0.62 | 0.52 | 0.8 | 5.64 | 0.0001 | 0.001 | 0.0014 |
| 2828637 | 0.64 | 0.0103 | 1.26 | 0.63 | 0.611 | 0.53 | 0.68 | 0.49 | 0.72 | 5.51 | 0.0074 | 0.0022 | 0.0082 |
| 2828642 | 0.65 | 0.0056 | 1.52 | 0.62 | 0.6786 | 0.57 | 0.65 | 0.48 | 0.73 | 4.5 | 0.0113 | 0.0054 | 0.0087 |
| 2829255 | 0.65 | 0.0036 | 1.34 | 0.63 | 0.611 | 0.66 | 0.61 | 0.49 | 0.76 | 4.35 | 0.0048 | 0.0043 | 0.0056 |
| 2829555 | 0.57 | 0.1609 | 1.24 | 0.63 | 0.611 | 0.49 | 0.71 | 0.49 | 0.71 | 5.03 | 0.0191 | 0.0538 | 0.0889 |
| 2829679 | 0.65 | 0.006 | 1.33 | 0.64 | 0.5397 | 0.55 | 0.68 | 0.5 | 0.73 | 6.03 | 0.0078 | 0.0027 | 0.0045 |
| 2831231 | 0.64 | 0.0082 | 1.36 | 0.64 | 0.5397 | 0.57 | 0.67 | 0.5 | 0.73 | 6.17 | 0.0035 | 0.0071 | 0.0109 |
| 2831557 | 0.64 | 0.0076 | 1.4 | 0.6 | 0.7956 | 0.6 | 0.61 | 0.47 | 0.72 | 6.57 | 0.0219 | 0.0039 | 0.0047 |
| 2836687 | 0.63 | 0.0149 | 1.32 | 0.57 | 0.9388 | 0.66 | 0.52 | 0.44 | 0.73 | 4.88 | 0.0462 | 0.0081 | 0.0117 |
| 2840638 | 0.64 | 0.0094 | 1.31 | 0.61 | 0.7405 | 0.6 | 0.62 | 0.47 | 0.73 | 4.59 | 0.0129 | 0.0058 | 0.012 |
| 2843280 | 0.61 | 0.0373 | 1.33 | 0.57 | 0.9388 | 0.55 | 0.59 | 0.43 | 0.7 | 6.04 | 0.0824 | 0.028 | 0.0329 |
| 2844248 | 0.62 | 0.0187 | 1.52 | 0.64 | 0.4669 | 0.66 | 0.63 | 0.51 | 0.76 | 6.16 | 0.0012 | 0.0078 | 0.0093 |
| 2844250 | 0.64 | 0.0079 | 1.45 | 0.6 | 0.7956 | 0.66 | 0.57 | 0.47 | 0.75 | 5.75 | 0.0115 | 0.0063 | 0.005 |
| 2848258 | 0.65 | 0.0039 | 1.35 | 0.6 | 0.8429 | 0.47 | 0.67 | 0.45 | 0.69 | 4.95 | 0.0619 | 0.003 | 0.0062 |
| 2848472 | 0.62 | 0.0278 | 1.67 | 0.64 | 0.5397 | 0.57 | 0.67 | 0.5 | 0.73 | 6.16 | 0.003 | 0.0097 | 0.014 |
| 2851973 | 0.66 | 0.0027 | 1.32 | 0.64 | 0.4669 | 0.62 | 0.66 | 0.51 | 0.75 | 4.77 | 0.0017 | 0.0024 | 0.0039 |
| 2854808 | 0.65 | 0.0036 | 1.65 | 0.64 | 0.5397 | 0.68 | 0.61 | 0.5 | 0.77 | 5.98 | 0.0013 | 0.0017 | 0.0029 |
| 2854809 | 0.67 | 0.0019 | 1.29 | 0.66 | 0.3262 | 0.66 | 0.66 | 0.53 | 0.77 | 8.1 | 0.0002 | 0.0007 | 0.0016 |
| 2855289 | 0.65 | 0.0043 | 1.55 | 0.64 | 0.4669 | 0.62 | 0.66 | 0.51 | 0.75 | 5.36 | 0.0037 | 0.0058 | 0.0065 |
| 2855291 | 0.66 | 0.0024 | 1.49 | 0.66 | 0.3262 | 0.6 | 0.7 | 0.53 | 0.75 | 4.63 | 0.0016 | 0.0023 | 0.0029 |
| 2855292 | 0.64 | 0.0083 | 1.33 | 0.67 | 0.2627 | 0.64 | 0.68 | 0.54 | 0.77 | 5.55 | 0.0004 | 0.0029 | 0.0047 |
| 2857172 | 0.65 | 0.0054 | 1.58 | 0.64 | 0.4669 | 0.64 | 0.65 | 0.51 | 0.76 | 4.95 | 0.001 | 0.0029 | 0.0057 |
| 2862162 | 0.63 | 0.0166 | 1.26 | 0.64 | 0.4669 | 0.62 | 0.66 | 0.51 | 0.75 | 4.52 | 0.0014 | 0.0174 | 0.0248 |
| 2862377 | 0.64 | 0.0101 | 1.32 | 0.66 | 0.3262 | 0.7 | 0.63 | 0.52 | 0.79 | 7.96 | 0.0003 | 0.0059 | 0.007 |
| 2865063 | 0.64 | 0.0065 | 1.48 | 0.64 | 0.5397 | 0.7 | 0.6 | 0.5 | 0.78 | 6.19 | 0.001 | 0.0039 | 0.0059 |
| 2868141 | 0.64 | 0.0107 | 1.32 | 0.64 | 0.4669 | 0.62 | 0.66 | 0.51 | 0.75 | 5.73 | 0.0014 | 0.0056 | 0.0098 |
| 2877518 | 0.62 | 0.0245 | 1.31 | 0.64 | 0.5397 | 0.57 | 0.67 | 0.5 | 0.73 | 5.76 | 0.0045 | 0.0137 | 0.0243 |
| 2878261 | 0.65 | 0.004 | 1.3 | 0.62 | 0.6786 | 0.68 | 0.59 | 0.48 | 0.76 | 5.74 | 0.0012 | 0.0014 | 0.004 |
| 2882121 | 0.65 | 0.0039 | 1.36 | 0.64 | 0.5397 | 0.66 | 0.62 | 0.5 | 0.76 | 7.59 | 0.0011 | 0.0017 | 0.0038 |
| 2883344 | 0.61 | 0.0351 | 1.26 | 0.58 | 0.914 | 0.47 | 0.65 | 0.43 | 0.68 | 4.55 | 0.2339 | 0.0421 | 0.0391 |
| 2893822 | 0.61 | 0.0451 | 1.22 | 0.6 | 0.8429 | 0.36 | 0.73 | 0.44 | 0.67 | 5.07 | 0.175 | 0.0408 | 0.0892 |
| 2893942 | 0.63 | 0.0173 | 1.37 | 0.63 | 0.611 | 0.64 | 0.62 | 0.49 | 0.75 | 6.32 | 0.0041 | 0.017 | 0.0172 |
| 2896978 | 0.66 | 0.0035 | 1.37 | 0.64 | 0.5397 | 0.49 | 0.72 | 0.5 | 0.71 | 4.84 | 0.0241 | 0.0057 | 0.0038 |
| 2898531 | 0.63 | 0.018 | 1.32 | 0.53 | 0.9955 | 0.6 | 0.49 | 0.4 | 0.68 | 5.35 | 0.2482 | 0.009 | 0.0177 |
| 2902010 | 0.65 | 0.0062 | 1.28 | 0.65 | 0.3949 | 0.6 | 0.68 | 0.52 | 0.75 | 6.35 | 0.0015 | 0.0032 | 0.0052 |
| 2902011 | 0.63 | 0.018 | 1.49 | 0.63 | 0.611 | 0.68 | 0.6 | 0.49 | 0.77 | 5.59 | 0.0034 | 0.0066 | 0.0099 |
| 2905137 | 0.67 | 0.0013 | 1.39 | 0.69 | 0.1164 | 0.55 | 0.77 | 0.58 | 0.75 | 4.9 | 0.0001 | 0.0001 | 0.0007 |
| 2906968 | 0.64 | 0.0077 | 1.4 | 0.64 | 0.5397 | 0.66 | 0.62 | 0.5 | 0.76 | 5.69 | 0.0025 | 0.0046 | 0.0059 |
| 2908048 | 0.61 | 0.0378 | 1.33 | 0.57 | 0.9577 | 0.62 | 0.54 | 0.43 | 0.71 | 4.61 | 0.0579 | 0.0322 | 0.0492 |
| 2909471 | 0.63 | 0.0121 | 1.41 | 0.61 | 0.7405 | 0.6 | 0.62 | 0.47 | 0.73 | 5 | 0.0179 | 0.0165 | 0.0182 |
| 2910489 | 0.65 | 0.0062 | 1.26 | 0.6 | 0.8429 | 0.7 | 0.54 | 0.46 | 0.76 | 5.05 | 0.0087 | 0.0081 | 0.0108 |
| 2914002 | 0.65 | 0.0063 | 1.33 | 0.64 | 0.4669 | 0.49 | 0.73 | 0.51 | 0.71 | 4.56 | 0.0061 | 0.0033 | 0.005 |
| 2914107 | 0.71 | 0.0001 | 1.28 | 0.68 | 0.1571 | 0.74 | 0.65 | 0.55 | 0.82 | 4.16 | 0 | 0.0011 | 0.0018 |
| 2916383 | 0.62 | 0.0221 | 1.24 | 0.58 | 0.914 | 0.64 | 0.55 | 0.45 | 0.73 | 5.73 | 0.0307 | 0.018 | 0.0257 |
| 2916976 | 0.68 | 0.0007 | 1.44 | 0.68 | 0.1571 | 0.57 | 0.74 | 0.56 | 0.75 | 4.23 | 0 | 0.0008 | 0.0013 |
| 2918543 | 0.64 | 0.0075 | 1.33 | 0.64 | 0.5397 | 0.66 | 0.62 | 0.5 | 0.76 | 4.64 | 0.002 | 0.0096 | 0.0077 |
| 2918547 | 0.66 | 0.0021 | 1.46 | 0.63 | 0.611 | 0.62 | 0.63 | 0.49 | 0.74 | 6.45 | 0.0042 | 0.002 | 0.0028 |
| 2922232 | 0.63 | 0.0137 | 1.27 | 0.62 | 0.6786 | 0.45 | 0.72 | 0.48 | 0.69 | 5.15 | 0.0405 | 0.024 | 0.0242 |
| 2922235 | 0.66 | 0.003 | 1.4 | 0.64 | 0.4669 | 0.51 | 0.77 | 0.51 | 0.72 | 4.63 | 0.008 | 0.0105 | 0.0083 |
| 2926222 | 0.67 | 0.0012 | 1.46 | 0.65 | 0.3949 | 0.7 | 0.62 | 0.52 | 0.78 | 7.58 | 0.0005 | 0.001 | 0.0012 |
| 2929946 | 0.65 | 0.0048 | 1.29 | 0.64 | 0.4669 | 0.53 | 0.71 | 0.51 | 0.73 | 5.67 | 0.003 | 0.0044 | 0.0065 |
| 3930652 | 0.65 | 0.0052 | 1.44 | 0.63 | 0.611 | 0.62 | 0.63 | 0.49 | 0.74 | 6.5 | 0.005 | 0.0045 | 0.0055 |
| 2932399 | 0.6 | 0.0612 | 1.24 | 0.62 | 0.6786 | 0.51 | 0.68 | 0.48 | 0.71 | 4.8 | 0.0367 | 0.0637 | 0.0539 |
| 2932428 | 0.65 | 0.0059 | 1.52 | 0.65 | 0.3949 | 0.53 | 0.72 | 0.52 | 0.73 | 4.44 | 0.0035 | 0.0056 | 0.007 |
| 2933660 | 0.66 | 0.002 | 1.27 | 0.67 | 0.206 | 0.7 | 0.66 | 0.54 | 0.79 | 5.57 | 0.0001 | 0.002 | 0.004 |
| 2936963 | 0.64 | 0.01 | 1.37 | 0.6 | 0.7956 | 0.4 | 0.72 | 0.45 | 0.68 | 3.97 | 0.1336 | 0.0447 | 0.057 |
| 2940827 | 0.65 | 0.0039 | 1.41 | 0.63 | 0.611 | 0.6 | 0.65 | 0.49 | 0.74 | 5.39 | 0.0044 | 0.0069 | 0.0076 |
| 2942512 | 0.62 | 0.0268 | 1.23 | 0.6 | 0.8429 | 0.49 | 0.66 | 0.45 | 0.69 | 5.96 | 0.056 | 0.0433 | 0.045 |
| 2944070 | 0.65 | 0.0059 | 1.47 | 0.63 | 0.611 | 0.6 | 0.65 | 0.49 | 0.74 | 5.68 | 0.0043 | 0.006 | 0.0075 |
| 2944963 | 0.68 | 0.0006 | 1.3 | 0.65 | 0.3949 | 0.62 | 0.67 | 0.52 | 0.75 | 7.11 | 0.0013 | 0.0004 | 0.0007 |
| 2946479 | 0.64 | 0.0071 | 1.4 | 0.64 | 0.4669 | 0.53 | 0.71 | 0.51 | 0.73 | 4.29 | 0.0076 | 0.0283 | 0.0289 |
| 2947592 | 0.66 | 0.0024 | 1.46 | 0.64 | 0.4669 | 0.6 | 0.67 | 0.51 | 0.74 | 4.7 | 0.0015 | 0.0005 | 0.0018 |
| 2948592 | 0.66 | 0.0031 | 1.33 | 0.6 | 0.8429 | 0.6 | 0.6 | 0.46 | 0.72 | 5.71 | 0.0251 | 0.0031 | 0.0039 |
| 2948596 | 0.65 | 0.0036 | 1.32 | 0.61 | 0.7405 | 0.68 | 0.57 | 0.48 | 0.76 | 4.3 | 0.0067 | 0.0052 | 0.0061 |
| 2949053 | 0.63 | 0.0173 | 1.41 | 0.63 | 0.611 | 0.6 | 0.65 | 0.49 | 0.74 | 4.92 | 0.0032 | 0.0108 | 0.0184 |
| 2949061 | 0.58 | 0.1101 | 1.27 | 0.6 | 0.8429 | 0.49 | 0.66 | 0.45 | 0.69 | 4.84 | 0.0833 | 0.0877 | 0.1248 |
| 2951060 | 0.63 | 0.0123 | 1.28 | 0.61 | 0.7405 | 0.7 | 0.56 | 0.48 | 0.77 | 6.58 | 0.0039 | 0.0073 | 0.0069 |
| 2956077 | 0.65 | 0.0046 | 1.3 | 0.64 | 0.4669 | 0.49 | 0.73 | 0.51 | 0.71 | 5.43 | 0.0069 | 0.0034 | 0.0085 |
| 2958402 | 0.65 | 0.0056 | 1.32 | 0.66 | 0.3262 | 0.55 | 0.72 | 0.53 | 0.74 | 5.07 | 0.0007 | 0.0143 | 0.0251 |
| 2961302 | 0.63 | 0.0149 | 1.4 | 0.62 | 0.6786 | 0.72 | 0.56 | 0.49 | 0.78 | 5.63 | 0.0018 | 0.0082 | 0.0107 |
| 2966293 | 0.63 | 0.0157 | 1.37 | 0.64 | 0.5397 | 0.55 | 0.68 | 0.5 | 0.73 | 4.28 | 0.0062 | 0.0149 | 0.0183 |
| 2966378 | 0.62 | 0.021 | 1.31 | 0.63 | 0.611 | 0.66 | 0.61 | 0.49 | 0.76 | 5.86 | 0.0035 | 0.0172 | 0.0181 |
| 2967164 | 0.63 | 0.0115 | 1.26 | 0.63 | 0.611 | 0.57 | 0.66 | 0.49 | 0.73 | 3.89 | 0.0053 | 0.0101 | 0.022 |
| 2967323 | 0.62 | 0.0258 | 1.43 | 0.64 | 0.4669 | 0.62 | 0.66 | 0.51 | 0.75 | 4.4 | 0.0038 | 0.0082 | 0.0127 |
| 2968257 | 0.61 | 0.0401 | 1.23 | 0.6 | 0.8429 | 0.57 | 0.61 | 0.46 | 0.71 | 5.01 | 0.0256 | 0.0424 | 0.0552 |
| 2968653 | 0.64 | 0.0103 | 1.37 | 0.63 | 0.611 | 0.57 | 0.66 | 0.49 | 0.73 | 5.14 | 0.0066 | 0.0094 | 0.0116 |
| 2974195 | 0.64 | 0.0094 | 1.25 | 0.6 | 0.7956 | 0.77 | 0.51 | 0.47 | 0.79 | 6.75 | 0.002 | 0.0091 | 0.0118 |
| 2974332 | 0.65 | 0.0041 | 1.67 | 0.64 | 0.5397 | 0.66 | 0.62 | 0.5 | 0.76 | 4.95 | 0.0036 | 0.0065 | 0.0052 |
| 2975686 | 0.67 | 0.0019 | 1.34 | 0.66 | 0.3262 | 0.68 | 0.65 | 0.52 | 0.78 | 5.93 | 0.0006 | 0.0017 | 0.0014 |
| 2975709 | 0.63 | 0.0166 | 1.27 | 0.62 | 0.6786 | 0.4 | 0.74 | 0.48 | 0.69 | 5.29 | 0.0671 | 0.0242 | 0.0287 |
| 2976368 | 0.63 | 0.0162 | 1.35 | 0.64 | 0.5397 | 0.47 | 0.73 | 0.5 | 0.71 | 5.49 | 0.012 | 0.0214 | 0.0372 |
| 2977521 | 0.64 | 0.0068 | 1.31 | 0.58 | 0.914 | 0.74 | 0.49 | 0.45 | 0.77 | 6.32 | 0.0093 | 0.0064 | 0.0074 |
| 2981918 | 0.63 | 0.0116 | 1.39 | 0.62 | 0.6786 | 0.51 | 0.68 | 0.48 | 0.71 | 5.26 | 0.0269 | 0.0118 | 0.0142 |
| 2982323 | 0.64 | 0.0098 | 1.39 | 0.63 | 0.611 | 0.68 | 0.6 | 0.49 | 0.77 | 6.6 | 0.0014 | 0.0067 | 0.008 |
| 2982385 | 0.68 | 0.001 | 1.48 | 0.63 | 0.611 | 0.6 | 0.65 | 0.49 | 0.74 | 4.49 | 0.0141 | 0.0011 | 0.0013 |
| 2984576 | 0.61 | 0.0303 | 1.39 | 0.61 | 0.7405 | 0.53 | 0.66 | 0.47 | 0.71 | 4.1 | 0.0315 | 0.0123 | 0.0164 |
| 2985813 | 0.64 | 0.0071 | 1.51 | 0.64 | 0.4669 | 0.55 | 0.7 | 0.51 | 0.73 | 4.14 | 0.0044 | 0.0268 | 0.0347 |
| 2985965 | 0.68 | 0.0006 | 1.28 | 0.66 | 0.3262 | 0.6 | 0.7 | 0.53 | 0.75 | 6.32 | 0.0007 | 0.0006 | 0.0007 |
| 2988895 | 0.66 | 0.0028 | 1.26 | 0.66 | 0.3262 | 0.68 | 0.65 | 0.52 | 0.78 | 4.25 | 0.0004 | 0.0048 | 0.0074 |
| 2988896 | 0.65 | 0.0039 | 1.31 | 0.58 | 0.914 | 0.74 | 0.49 | 0.45 | 0.77 | 7.44 | 0.0123 | 0.0049 | 0.005 |
| 2989720 | 0.66 | 0.0031 | 1.31 | 0.61 | 0.7405 | 0.47 | 0.7 | 0.47 | 0.7 | 5.09 | 0.0541 | 0.0021 | 0.0029 |
| 2991248 | 0.61 | 0.0307 | 0.26 | 0.62 | 0.6786 | 0.64 | 0.61 | 0.48 | 0.75 | 6.93 | 0.0056 | 0.0169 | 0.0238 |
| 2991263 | 0.68 | 0.0007 | 1.32 | 0.66 | 0.3262 | 0.68 | 0.65 | 0.52 | 0.78 | 6.56 | 0.0002 | 0.001 | 0.0017 |
| 2991264 | 0.65 | 0.0041 | 1.52 | 0.67 | 0.206 | 0.64 | 0.7 | 0.55 | 0.77 | 5.46 | 0.0001 | 0.0029 | 0.0046 |
| 2903622 | 0.64 | 0.007 | 1.32 | 0.6 | 0.8429 | 0.66 | 0.56 | 0.46 | 0.74 | 4.94 | 0.0201 | 0.0064 | 0.008 |
| 2993644 | 0.65 | 0.0054 | 1.32 | 0.6 | 0.8429 | 0.68 | 0.55 | 0.46 | 0.75 | 4.71 | 0.009 | 0.0037 | 0.0064 |
| 2993659 | 0.68 | 0.0007 | 1.48 | 0.67 | 0.2627 | 0.64 | 0.68 | 0.54 | 0.77 | 5.68 | 0.0004 | 0.0006 | 0.0009 |
| 2994371 | 0.62 | 0.0255 | 1.24 | 0.64 | 0.5397 | 0.49 | 0.72 | 0.5 | 0.71 | 3.95 | 0.0273 | 0.0382 | 0.0314 |
| 2995598 | 0.62 | 0.0242 | 1.25 | 0.6 | 0.8429 | 0.51 | 0.65 | 0.45 | 0.7 | 5.45 | 0.1016 | 0.0497 | 0.0309 |
| 2997143 | 0.67 | 0.0018 | 1.39 | 0.63 | 0.611 | 0.51 | 0.7 | 0.49 | 0.71 | 4.86 | 0.0105 | 0.0035 | 0.0077 |
| 3002667 | 0.64 | 0.0082 | 1.33 | 0.66 | 0.3262 | 0.64 | 0.67 | 0.53 | 0.76 | 6.08 | 0.0009 | 0.0073 | 0.0119 |
| 3003180 | 0.62 | 0.023 | 1.41 | 0.61 | 0.7405 | 0.51 | 0.67 | 0.47 | 0.71 | 4.09 | 0.0402 | 0.0098 | 0.013 |
| 3004652 | 0.68 | 0.0007 | 1.36 | 0.6 | 0.8429 | 0.38 | 0.72 | 0.44 | 0.67 | 5.1 | 0.2088 | 0.0054 | 0.0055 |
| 3011652 | 0.62 | 0.0213 | 1.28 | 0.65 | 0.3949 | 0.62 | 0.67 | 0.52 | 0.75 | 6.93 | 0.0014 | 0.0109 | 0.0145 |
| 3011846 | 0.62 | 0.0278 | 1.34 | 0.63 | 0.611 | 0.49 | 0.71 | 0.49 | 0.71 | 4.56 | 0.025 | 0.0101 | 0.0183 |
| 3012405 | 0.67 | 0.0011 | 1.25 | 0.67 | 0.206 | 0.66 | 0.68 | 0.54 | 0.78 | 6.88 | 0.0001 | 0.0008 | 0.0014 |
| 3012428 | 0.69 | 0.0003 | 1.24 | 0.62 | 0.6786 | 0.47 | 0.71 | 0.48 | 0.7 | 5.68 | 0.0424 | 0.0004 | 0.0015 |
| 3012438 | 0.61 | 0.0307 | 1.3 | 0.59 | 0.8822 | 0.45 | 0.67 | 0.44 | 0.68 | 4.06 | 0.2198 | 0.0154 | 0.02 |
| 3012452 | 0.64 | 0.0078 | 1.4 | 0.64 | 0.5397 | 0.51 | 0.71 | 0.5 | 0.72 | 4.94 | 0.0076 | 0.0029 | 0.0066 |
| 3012453 | 0.66 | 0.0024 | 1.33 | 0.67 | 0.206 | 0.62 | 0.71 | 0.55 | 0.76 | 6.58 | 0.0001 | 0.001 | 0.0025 |
| 3013190 | 0.63 | 0.0162 | 1.22 | 0.64 | 0.5397 | 0.47 | 0.73 | 0.5 | 0.71 | 4.93 | 0.0182 | 0.0196 | 0.0231 |
| 3013468 | 0.67 | 0.0019 | 1.46 | 0.64 | 0.5397 | 0.62 | 0.65 | 0.5 | 0.75 | 5.44 | 0.0023 | 0.0013 | 0.0022 |
| 3013685 | 0.61 | 0.0351 | 1.29 | 0.62 | 0.6786 | 0.51 | 0.68 | 0.48 | 0.71 | 5.2 | 0.0298 | 0.032 | 0.0298 |
| 3014422 | 0.6 | 0.0573 | 1.12 | 0.64 | 0.4669 | 0.43 | 0.77 | 0.51 | 0.7 | 4.18 | 0.0118 | 0.018 | 0.0587 |
| 3015985 | 0.64 | 0.0072 | 1.29 | 0.64 | 0.4669 | 0.64 | 0.65 | 0.51 | 0.76 | 7.05 | 0.001 | 0.0053 | 0.0069 |
| 3017629 | 0.63 | 0.0153 | 1.36 | 0.61 | 0.7405 | 0.36 | 0.76 | 0.46 | 0.67 | 3.67 | 0.1401 | 0.0342 | 0.0398 |
| 3017826 | 0.63 | 0.0159 | 1.43 | 0.62 | 0.6786 | 0.62 | 0.62 | 0.48 | 0.74 | 5.59 | 0.0059 | 0.0127 | 0.0135 |
| 3021760 | 0.69 | 0.0003 | 1.4 | 0.71 | 0.0394 | 0.62 | 0.77 | 0.6 | 0.78 | 4.95 | 0 | 0.0001 | 0.0005 |
| 3022682 | 0.65 | 0.0057 | 1.29 | 0.65 | 0.3949 | 0.66 | 0.65 | 0.52 | 0.77 | 6.44 | 0.0005 | 0.0043 | 0.0048 |
| 3027555 | 0.66 | 0.0025 | 1.57 | 0.62 | 0.6786 | 0.74 | 0.55 | 0.49 | 0.79 | 6.4 | 0.0011 | 0.0016 | 0.0021 |
| 3027926 | 0.65 | 0.0039 | 1.32 | 0.64 | 0.5397 | 0.57 | 0.67 | 0.5 | 0.73 | 5.95 | 0.0062 | 0.0033 | 0.005 |
| 3027936 | 0.64 | 0.0104 | 1.11 | 0.6 | 0.7956 | 0.74 | 0.52 | 0.47 | 0.78 | 6.19 | 0.0039 | 0.0071 | 0.0078 |
| 3037391 | 0.67 | 0.0018 | 1.41 | 0.63 | 0.611 | 0.49 | 0.71 | 0.49 | 0.71 | 4 | 0.0191 | 0.0109 | 0.011 |
| 3037406 | 0.64 | 0.0085 | 1.34 | 0.63 | 0.611 | 0.72 | 0.57 | 0.49 | 0.78 | 7.3 | 0.0014 | 0.0052 | 0.0096 |
| 3037950 | 0.63 | 0.0145 | 1.35 | 0.63 | 0.611 | 0.47 | 0.72 | 0.49 | 0.7 | 4.07 | 0.018 | 0.0129 | 0.0227 |
| 3039980 | 0.65 | 0.0062 | 1.41 | 0.67 | 0.2627 | 0.64 | 0.68 | 0.54 | 0.77 | 6.48 | 0.0001 | 0.0022 | 0.0055 |
| 3039983 | 0.62 | 0.0221 | 1.49 | 0.6 | 0.7956 | 0.62 | 0.6 | 0.47 | 0.73 | 5.59 | 0.0126 | 0.0115 | 0.0157 |
| 3040116 | 0.64 | 0.0088 | 1.49 | 0.66 | 0.3262 | 0.51 | 0.74 | 0.53 | 0.73 | 4.93 | 0.0022 | 0.0076 | 0.012 |
| 3041262 | 0.67 | 0.0011 | 1.53 | 0.64 | 0.5397 | 0.62 | 0.65 | 0.5 | 0.75 | 6.49 | 0.0024 | 0.0007 | 0.0012 |
| 3042004 | 0.64 | 0.007 | 1.41 | 0.67 | 0.2627 | 0.68 | 0.66 | 0.53 | 0.78 | 5.3 | 0.0001 | 0.0062 | 0.009 |
| 3042421 | 0.66 | 0.0026 | 1.71 | 0.66 | 0.3262 | 0.66 | 0.66 | 0.53 | 0.77 | 5.87 | 0.0005 | 0.0017 | 0.0018 |
| 3042426 | 0.65 | 0.0048 | 1.39 | 0.66 | 0.3262 | 0.7 | 0.63 | 0.52 | 0.79 | 5.98 | 0.0003 | 0.0026 | 0.0037 |
| 3042429 | 0.62 | 0.0213 | 1.27 | 0.61 | 0.7405 | 0.51 | 0.67 | 0.47 | 0.71 | 4.72 | 0.0405 | 0.0366 | 0.0432 |
| 3042430 | 0.65 | 0.004 | 1.65 | 0.63 | 0.611 | 0..68 | 0.6 | 0.49 | 0.77 | 5.36 | 0.002 | 0.0026 | 0.0026 |
| 3042437 | 0.71 | 0.0001 | 1.4 | 0.68 | 0.1571 | 0.68 | 0.68 | 0.55 | 0.79 | 6.83 | 0.0001 | 0.0001 | 0.0001 |
| 3042453 | 0.64 | 0.0085 | 1.35 | 0.64 | 0.4669 | 0.53 | 0.71 | 0.51 | 0.73 | 3.9 | 0.0064 | 0.0108 | 0.0159 |
| 3048780 | 0.65 | 0.0052 | 1.51 | 0.6 | 0.7956 | 0.6 | 0.61 | 0.47 | 0.72 | 4.61 | 0.0194 | 0.0044 | 0.0067 |
| 3048870 | 0.66 | 0.0022 | 1.36 | 0.65 | 0.3949 | 0.55 | 0.71 | 0.5 | 0.73 | 5.03 | 0.0027 | 0.0027 | 0.0028 |
| 3053384 | 0.64 | 0.009 | 1.44 | 0.64 | 0.5397 | 0.47 | 0.73 | 0.5 | 0.71 | 3.82 | 0.0193 | 0.0009 | 0.0025 |
| 3056395 | 0.65 | 0.0044 | 1.38 | 0.64 | 0.4669 | 0.66 | 0.63 | 0.51 | 0.76 | 7.19 | 0.0008 | 0.0028 | 0.0047 |
| 3060052 | 0.68 | 0.0008 | 1.21 | 0.64 | 0.5397 | 0.47 | 0.73 | 0.5 | 0.71 | 3.77 | 0.0123 | 0.0019 | 0.0037 |
| 3061054 | 0.68 | 0.0009 | 1.28 | 0.66 | 0.3262 | 0.51 | 0.74 | 0.53 | 0.73 | 4.74 | 0.0019 | 0.0014 | 0.0015 |
| 3061144 | 0.62 | 0.0242 | 1.42 | 0.65 | 0.3949 | 0.55 | 0.71 | 0.52 | 0.73 | 4.87 | 0.0031 | 0.0241 | 0.0325 |
| 3061758 | 0.66 | 0.0032 | 1.34 | 0.64 | 0.4669 | 0.6 | 0.67 | 0.51 | 0.74 | 5.24 | 0.0014 | 0.0081 | 0.0219 |
| 3062023 | 0.68 | 0.0009 | 1.29 | 0.68 | 0.1571 | 0.57 | 0.74 | 0.56 | 0.75 | 5.88 | 0.0002 | 0.0002 | 0.0007 |
| 3064354 | 0.65 | 0.0055 | 1.32 | 0.62 | 0.6786 | 0.66 | 0.6 | 0.48 | 0.75 | 5.87 | 0.0046 | 0.0043 | 0.0049 |
| 3075645 | 0.61 | 0.0461 | 1.39 | 0.6 | 0.7956 | 0.64 | 0.59 | 0.47 | 0.74 | 5.92 | 0.0083 | 0.0323 | 0.043 |
| 3076355 | 0.71 | 0.0001 | 1.56 | 0.65 | 0.3949 | 0.57 | 0.7 | 0.52 | 0.74 | 5.9 | 0.0017 | 0.0001 | 0.0001 |
| 3076393 | 0.61 | 0.0477 | 1.25 | 0.64 | 0.4669 | 0.51 | 0.72 | 0.51 | 0.72 | 4.07 | 0.0035 | 0.006 | 0.024 |
| 3076490 | 0.66 | 0.0031 | 1.31 | 0.65 | 0.3949 | 0.64 | 0.66 | 0.52 | 0.76 | 6.63 | 0.0011 | 0.0029 | 0.0027 |
| 3081625 | 0.62 | 0.0197 | 1.21 | 0.61 | 0.7405 | 0.49 | 0.68 | 0.47 | 0.7 | 5.04 | 0.0337 | 0.0079 | 0.0115 |
| 3087828 | 0.68 | 0.0008 | 1.42 | 0.65 | 0.3949 | 0.6 | 0.68 | 0.52 | 0.75 | 5.2 | 0.0012 | 0.0004 | 0.0009 |
| 3087930 | 0.65 | 0.0039 | 1.47 | 0.66 | 0.3262 | 0.6 | 0.7 | 0.53 | 0.75 | 5.85 | 0.0015 | 0.005 | 0.0046 |
| 3088570 | 0.64 | 0.0079 | 1.41 | 0.62 | 0.6786 | 0.66 | 0.6 | 0.48 | 0.75 | 5.46 | 0.0049 | 0.0097 | 0.0089 |
| 3092505 | 0.63 | 0.0112 | 1.44 | 0.6 | 0.8429 | 0.66 | 0.56 | 0.46 | 0.74 | 5.61 | 0.0182 | 0.0088 | 0.0101 |
| 3094309 | 0.66 | 0.003 | 1.7 | 0.6 | 0.8429 | 0.57 | 0.61 | 0.46 | 0.71 | 4.94 | 0.0336 | 0.002 | 0.0029 |
| 3096162 | 0.66 | 0.0028 | 1.41 | 0.69 | 0.1164 | 0.68 | 0.7 | 0.56 | 0.79 | 4.95 | 0.0001 | 0.0029 | 0.003 |
| 3096409 | 0.68 | 0.0009 | 1.55 | 0.7 | 0.0837 | 0.53 | 0.79 | 0.6 | 0.75 | 4.94 | 0.0001 | 0.0008 | 0.0016 |
| 3096979 | 0.68 | 0.0008 | 1.38 | 0.67 | 0.206 | 0.64 | 0.7 | 0.55 | 0.77 | 5.5 | 0.0001 | 0.0008 | 0.0015 |
| 3099780 | 0.65 | 0.0046 | 1.44 | 0.63 | 0.611 | 0.64 | 0.62 | 0.49 | 0.75 | 5.78 | 0.0064 | 0.0041 | 0.0034 |
| 3100230 | 0.65 | 0.0048 | 1.31 | 0.64 | 0.5397 | 0.62 | 0.65 | 0.5 | 0.75 | 5.87 | 0.0036 | 0.0034 | 0.0049 |
| 3107638 | 0.65 | 0.0049 | 1.32 | 0.64 | 0.5397 | 0.45 | 0.74 | 0.5 | 0.7 | 4.51 | 0.005 | 0.0014 | 0.0068 |
| 3108472 | 0.69 | 0.0003 | 1.35 | 0.67 | 0.2627 | 0.66 | 0.67 | 0.53 | 0.77 | 6.54 | 0.0002 | 0.0003 | 0.0007 |
| 3108501 | 0.69 | 0.0002 | 1.35 | 0.66 | 0.3262 | 0.6 | 0.7 | 0.53 | 0.75 | 4.16 | 0.0017 | 0.0005 | 0.0009 |
| 3108938 | 0.62 | 0.0216 | 1.28 | 0.63 | 0.611 | 0.62 | 0.63 | 0.49 | 0.74 | 4.56 | 0.003 | 0.0125 | 0.0266 |
| 3111090 | 0.64 | 0.009 | 1.28 | 0.66 | 0.3262 | 0.55 | 0.72 | 0.53 | 0.74 | 5.59 | 0.0012 | 0.0054 | 0.0113 |
| 3111116 | 0.6 | 0.0489 | 1.42 | 0.63 | 0.611 | 0.62 | 0.63 | 0.49 | 0.74 | 5.08 | 0.003 | 0.0154 | 0.0273 |
| 3112517 | 0.68 | 0.0008 | 1.42 | 0.64 | 0.4669 | 0.6 | 0.67 | 0.51 | 0.74 | 6.38 | 0.0016 | 0.0003 | 0.0007 |
| 3114370 | 0.62 | 0.0194 | 1.3 | 0.64 | 0.4669 | 0.51 | 0.72 | 0.51 | 0.72 | 4.48 | 0.0061 | 0.0088 | 0.0157 |
| 3114664 | 0.63 | 0.0162 | 1.29 | 0.62 | 0.6786 | 0.53 | 0.67 | 0.48 | 0.71 | 5.21 | 0.0142 | 0.0089 | 0.0132 |
| 3124536 | 0.65 | 0.0044 | 1.28 | 0.64 | 0.4669 | 0.62 | 0.66 | 0.51 | 0.75 | 6.5 | 0.0033 | 0.0049 | 0.0055 |
| 3126094 | 0.66 | 0.0024 | 1.44 | 0.63 | 0.611 | 0.77 | 0.55 | 0.49 | 0.8 | 7.1 | 0.0008 | 0.0019 | 0.0019 |
| 3128680 | 0.6 | 0.0145 | 1.32 | 0.58 | 0.914 | 0.53 | 0.61 | 0.44 | 0.69 | 5.38 | 0.1137 | 0.019 | 0.0232 |
| 3129955 | 0.66 | 0.0028 | 1.36 | 0.64 | 0.4669 | 0.62 | 0.66 | 0.51 | 0.75 | 5.98 | 0.0015 | 0.0023 | 0.0044 |
| 3131973 | 0.67 | 0.001 | 1.36 | 0.69 | 0.1164 | 0.6 | 0.74 | 0.57 | 0.76 | 6.31 | 0.0001 | 0.0009 | 0.0014 |
| 3132336 | 0.64 | 0.0088 | 1.48 | 0.66 | 0.3262 | 0.57 | 0.71 | 0.53 | 0.74 | 4.37 | 0.001 | 0.0078 | 0.011 |
| 3133528 | 0.71 | 0.0001 | 1.48 | 0.69 | 0.1164 | 0.55 | 0.77 | 0.58 | 0.75 | 4.42 | 0.0001 | 0 | 0.0002 |
| 3134184 | 0.58 | 0.1565 | 1.14 | 0.6 | 0.7956 | 0.3 | 0.78 | 0.44 | 0.66 | 3.84 | 0.2446 | 0.0721 | 0.1183 |
| 3136352 | 0.65 | 0.0046 | 1.3 | 0.6 | 0.8429 | 0.62 | 0.59 | 0.46 | 0.73 | 6.73 | 0.0216 | 0.0027 | 0.0052 |
| 3137586 | 0.68 | 0.0006 | 1.39 | 0.62 | 0.6786 | 0.68 | 0.59 | 0.48 | 0.76 | 6.25 | 0.0032 | 0.0007 | 0.001 |
| 3138886 | 0.63 | 0.0135 | 1.31 | 0.6 | 0.8429 | 0.57 | 0.61 | 0.46 | 0.71 | 5.27 | 0.0363 | 0.013 | 0.0198 |
| 3138980 | 0.64 | 0.0076 | 1.36 | 0.65 | 0.3949 | 0.64 | 0.66 | 0.52 | 0.76 | 5.71 | 0.0007 | 0.0066 | 0.0076 |
| 3139053 | 0.66 | 0.0026 | 1.34 | 0.67 | 0.2627 | 0.55 | 0.73 | 0.54 | 0.74 | 5.25 | 0.0009 | 0.0048 | 0.0066 |
| 3139926 | 0.66 | 0.0021 | 1.35 | 0.65 | 0.3949 | 0.53 | 0.72 | 0.52 | 0.73 | 5.76 | 0.0026 | 0.0026 | 0.0041 |
| 3141863 | 0.66 | 0.0019 | 1.69 | 0.66 | 0.3262 | 0.62 | 0.68 | 0.53 | 0.76 | 3.82 | 0.0005 | 0.002 | 0.003 |
| 3145023 | 0.62 | 0.02 | 1.34 | 0.64 | 0.5397 | 0.53 | 0.7 | 0.5 | 0.72 | 5.2 | 0.0102 | 0.014 | 0.0172 |
| 3145956 | 0.66 | 0.002 | 1.33 | 0.64 | 0.4669 | 0.72 | 0.6 | 0.51 | 0.79 | 8.36 | 0.0006 | 0.0039 | 0.0057 |
| 3145966 | 0.65 | 0.0046 | 1.25 | 0.64 | 0.4669 | 0.64 | 0.65 | 0.51 | 0.76 | 5.71 | 0.0012 | 0.0033 | 0.006 |
| 3146538 | 0.65 | 0.006 | 1.33 | 0.62 | 0.6786 | 0.49 | 0.7 | 0.48 | 0.7 | 3.95 | 0.0239 | 0.0116 | 0.0213 |
| 3146566 | 0.64 | 0.0075 | 1.48 | 0.69 | 0.1164 | 0.57 | 0.76 | 0.57 | 0.76 | 5.22 | 0.0001 | 0.0037 | 0.0074 |
| 3146788 | 0.62 | 0.0194 | 1.34 | 0.61 | 0.7405 | 0.64 | 0.6 | 0.48 | 0.74 | 7.33 | 0.0099 | 0.0095 | 0.0151 |
| 3146799 | 0.67 | 0.0014 | 1.3 | 0.64 | 0.5397 | 0.72 | 0.59 | 0.5 | 0.79 | 8.32 | 0.0009 | 0.0011 | 0.0013 |
| 3147328 | 0.63 | 0.0121 | 1.34 | 0.62 | 0.6786 | 0.62 | 0.62 | 0.48 | 0.74 | 4.93 | 0.0058 | 0.0062 | 0.0144 |
| 3148628 | 0.63 | 0.012 | 1.53 | 0.66 | 0.3262 | 0.51 | 0.74 | 0.53 | 0.73 | 4.22 | 0.0014 | 0.0064 | 0.0118 |
| 3149768 | 0.64 | 0.0092 | 1.46 | 0.65 | 0.3949 | 0.64 | 0.66 | 0.52 | 0.76 | 6.19 | 0.0008 | 0.0019 | 0.0065 |
| 3149773 | 0.68 | 0.0009 | 1.45 | 0.65 | 0.3949 | 0.64 | 0.66 | 0.52 | 0.76 | 4.72 | 0.001 | 0.0004 | 0.0011 |
| 3149864 | 0.67 | 0.0016 | 1.53 | 0.67 | 0.2627 | 0.72 | 0.63 | 0.53 | 0.8 | 5.14 | 0.0001 | 0.0011 | 0.0019 |
| 3151480 | 0.66 | 0.0031 | 1.84 | 0.65 | 0.3949 | 0.6 | 0.68 | 0.52 | 0.75 | 5.11 | 0.0013 | 0.0008 | 0.0016 |
| 3152560 | 0.61 | 0.0378 | 1.31 | 0.64 | 0.4669 | 0.55 | 0.7 | 0.51 | 0.73 | 4.37 | 0.0049 | 0.0181 | 0.0211 |
| 3153532 | 0.62 | 0.0285 | 1.32 | 0.64 | 0.5397 | 0.55 | 0.68 | 0.5 | 0.73 | 5.02 | 0.0088 | 0.0236 | 0.0297 |
| 3157388 | 0.64 | 0.0067 | 1.49 | 0.61 | 0.7405 | 0.66 | 0.59 | 0.48 | 0.75 | 6.08 | 0.007 | 0.0058 | 0.0066 |
| 3157461 | 0.64 | 0.01 | 1.61 | 0.6 | 0.7956 | 0.64 | 0.59 | 0.47 | 0.74 | 5.37 | 0.0123 | 0.0074 | 0.01 |
| 3157723 | 0.64 | 0.0084 | 1.4 | 0.64 | 0.5397 | 0.66 | 0.62 | 0.5 | 0.76 | 5.9 | 0.0017 | 0.0043 | 0.0068 |
| 3157847 | 0.66 | 0.0029 | 1.39 | 0.63 | 0.611 | 0.53 | 0.68 | 0.49 | 0.72 | 5.48 | 0.0188 | 0.0036 | 0.0035 |
| 3164299 | 0.64 | 0.0066 | 1.31 | 0.65 | 0.3949 | 0.43 | 0.78 | 0.53 | 0.7 | 5.08 | 0.0065 | 0.0123 | 0.0189 |
| 3165138 | 0.64 | 0.0068 | 1.44 | 0.6 | 0.8429 | 0.66 | 0.56 | 0.46 | 0.74 | 3.75 | 0.0136 | 0.0063 | 0.0097 |
| 3165799 | 0.69 | 0.0005 | 1.48 | 0.66 | 0.3262 | 0.6 | 0.7 | 0.53 | 0.75 | 5.16 | 0.0009 | 0.0005 | 0.0007 |
| 3166029 | 0.65 | 0.0052 | 0.81 | 0.4 | 1 | 0.4 | 0.39 | 0.28 | 0.53 | 4.13 | 0.0174 | 0.0086 | 0.01 |
| 3166735 | 0.66 | 0.0032 | 1.54 | 0.63 | 0.611 | 0.64 | 0.62 | 0.49 | 0.75 | 5.57 | 0.0039 | 0.0053 | 0.0066 |
| 3167990 | 0.69 | 0.0004 | 1.58 | 0.67 | 0.206 | 0.55 | 0.74 | 0.55 | 0.74 | 6.17 | 0.0007 | 0.0004 | 0.0006 |
| 3168127 | 0.6 | 0.0698 | 1.27 | 0.64 | 0.4669 | 0.49 | 0.73 | 0.51 | 0.71 | 3.79 | 0.0102 | 0.0707 | 0.0664 |
| 3169011 | 0.69 | 0.0004 | 0.83 | 0.41 | 1 | 0.23 | 0.51 | 0.22 | 0.54 | 3.15 | 0.0077 | 0.0004 | 0.0006 |
| 3174268 | 0.7 | 0.0002 | 1.28 | 0.67 | 0.2627 | 0.51 | 0.76 | 0.55 | 0.73 | 5.23 | 0.0007 | 0.0002 | 0.0007 |
| 3174441 | 0.65 | 0.0036 | 1.4 | 0.64 | 0.4669 | 0.57 | 0.68 | 0.51 | 0.74 | 5.29 | 0.0035 | 0.007 | 0.0081 |
| 3175632 | 0.66 | 0.0021 | 1.48 | 0.61 | 0.7405 | 0.7 | 0.56 | 0.48 | 0.77 | 4.82 | 0.0038 | 0.0021 | 0.0034 |
| 3175657 | 0.61 | 0.0331 | 1.17 | 0.6 | 0.7956 | 0.49 | 0.67 | 0.46 | 0.7 | 5.56 | 0.0853 | 0.0589 | 0.0601 |
| 3178635 | 0.66 | 0.0034 | 1.52 | 0.64 | 0.4669 | 0.57 | 0.68 | 0.51 | 0.74 | 4.07 | 0.0034 | 0.0076 | 0.011 |
| 3183178 | 0.62 | 0.0205 | 1.52 | 0.61 | 0.7405 | 0.53 | 0.66 | 0.47 | 0.71 | 4.5 | 0.0269 | 0.0077 | 0.0146 |
| 3190449 | 0.61 | 0.0369 | 1.38 | 0.6 | 0.8429 | 0.57 | 0.61 | 0.46 | 0.71 | 6.01 | 0.0237 | 0.0196 | 0.0375 |
| 3192674 | 0.62 | 0.0202 | 1.27 | 0.6 | 0.7956 | 0.4 | 0.72 | 0.45 | 0.68 | 5.28 | 0.0966 | 0.0252 | 0.039 |
| 3197644 | 0.64 | 0.0098 | 1.48 | 0.58 | 0.914 | 0.64 | 0.55 | 0.45 | 0.73 | 5.54 | 0.0283 | 0.0057 | 0.0086 |
| 3199457 | 0.65 | 0.0057 | 1.49 | 0.65 | 0.3949 | 0.6 | 0.68 | 0.52 | 0.75 | 4.81 | 0.0031 | 0.0032 | 0.004 |
| 3199485 | 0.63 | 0.0126 | 1.19 | 0.64 | 0.5397 | 0.43 | 0.76 | 0.5 | 0.7 | 4.52 | 0.0271 | 0.0073 | 0.0117 |
| 3200638 | 0.66 | 0.0032 | 1.68 | 0.64 | 0.4669 | 0.62 | 0.66 | 0.51 | 0.75 | 4.31 | 0.0008 | 0.0014 | 0.0031 |
| 3200711 | 0.62 | 0.0208 | 1.25 | 0.63 | 0.611 | 0.53 | 0.68 | 0.49 | 0.72 | 5.61 | 0.0125 | 0.0213 | 0.0227 |
| 3200718 | 0.66 | 0.0026 | 1.25 | 0.62 | 0.6786 | 0.62 | 0.62 | 0.48 | 0.74 | 6.15 | 0.0095 | 0.0019 | 0.0035 |
| 3200725 | 0.66 | 0.002 | 1.34 | 0.62 | 0.6786 | 0.68 | 0.59 | 0.48 | 0.76 | 6.37 | 0.0035 | 0.0013 | 0.0023 |
| 3203822 | 0.66 | 0.0032 | 1.31 | 0.61 | 0.7405 | 0.66 | 0.59 | 0.48 | 0.75 | 4.83 | 0.0072 | 0.0045 | 0.0063 |
| 3205039 | 0.63 | 0.0112 | 1.23 | 0.64 | 0.4669 | 0.6 | 0.67 | 0.51 | 0.74 | 5.79 | 0.0019 | 0.0132 | 0.0184 |
| 3205424 | 0.64 | 0.0064 | 1.38 | 0.65 | 0.3949 | 0.57 | 0.7 | 0.52 | 0.74 | 4.05 | 0.003 | 0.0104 | 0.009 |
| 3212146 | 0.66 | 0.0026 | 1.55 | 0.68 | 0.1571 | 0.64 | 0.71 | 0.56 | 0.77 | 5.88 | 0.0001 | 0.0008 | 0.0015 |
| 3212163 | 0.61 | 0.0315 | 1.4 | 0.63 | 0.611 | 0.6 | 0.65 | 0.49 | 0.74 | 4.68 | 0.0069 | 0.0117 | 0.0215 |
| 3214671 | 0.67 | 0.0016 | 1.33 | 0.64 | 0.5397 | 0.68 | 0.61 | 0.5 | 0.77 | 4.47 | 0.0021 | 0.0019 | 0.004 |
| 3214699 | 0.64 | 0.0076 | 1.29 | 0.67 | 0.2627 | 0.55 | 0.73 | 0.54 | 0.74 | 4.43 | 0.0007 | 0.0142 | 0.0278 |
| 3219683 | 0.64 | 0.008 | 1.34 | 0.61 | 0.7405 | 0.57 | 0.63 | 0.47 | 0.72 | 4.71 | 0.0234 | 0.0105 | 0.0178 |
| 3219711 | 0.66 | 0.0021 | 1.54 | 0.65 | 0.3949 | 0.51 | 0.73 | 0.52 | 0.72 | 3.71 | 0.0077 | 0.0011 | 0.004 |
| 3219891 | 0.67 | 0.0012 | 1.74 | 0.62 | 0.6786 | 0.6 | 0.63 | 0.48 | 0.73 | 5.21 | 0.0084 | 0.0012 | 0.0026 |
| 3220159 | 0.65 | 0.0062 | 1.42 | 0.62 | 0.6786 | 0.68 | 0.59 | 0.48 | 0.76 | 7.05 | 0.0028 | 0.006 | 0.0071 |
| 3222054 | 0.66 | 0.0027 | 1.42 | 0.67 | 0.206 | 0.51 | 0.77 | 0.56 | 0.73 | 5.59 | 0.0009 | 0.0016 | 0.0026 |
| 3222984 | 0.64 | 0.0075 | 1.3 | 0.61 | 0.7405 | 0.43 | 0.72 | 0.47 | 0.69 | 4.5 | 0.0689 | 0.0069 | 0.0103 |
| 3223876 | 0.65 | 0.0043 | 1.26 | 0.64 | 0.5397 | 0.57 | 0.67 | 0.5 | 0.73 | 5 | 0.0045 | 0.0042 | 0.0071 |
| 3225580 | 0.63 | 0.0128 | 1.26 | 0.61 | 0.7405 | 0.36 | 0.76 | 0.46 | 0.67 | 3.76 | 0.2566 | 0.0745 | 0.0409 |
| 3226470 | 0.63 | 0.0153 | 1.29 | 0.64 | 0.4669 | 0.57 | 0.68 | 0.51 | 0.74 | 3.35 | 0.0021 | 0.0092 | 0.0247 |
| 3227646 | 0.64 | 0.0097 | 1.4 | 0.62 | 0.6786 | 0.55 | 0.66 | 0.48 | 0.72 | 6.22 | 0.0143 | 0.0083 | 0.009 |
| 3233387 | 0.66 | 0.0022 | 1.67 | 0.66 | 0.3262 | 0.66 | 0.66 | 0.53 | 0.77 | 5.74 | 0.0004 | 0.0016 | 0.0014 |
| 3234175 | 0.63 | 0.0141 | 1.34 | 0.64 | 0.5397 | 0.7 | 0.6 | 0.5 | 0.78 | 6.78 | 0.0009 | 0.0075 | 0.0112 |
| 3235500 | 0.65 | 0.0052 | 1.32 | 0.64 | 0.5397 | 0.6 | 0.66 | 0.5 | 0.74 | 4.55 | 0.0052 | 0.0067 | 0.0072 |
| 3237000 | 0.63 | 0.0155 | 1.42 | 0.61 | 0.7405 | 0.68 | 0.57 | 0.48 | 0.76 | 6.47 | 0.0066 | 0.013 | 0.0135 |
| 3237001 | 0.66 | 0.0032 | 1.37 | 0.62 | 0.6786 | 0.7 | 0.57 | 0.49 | 0.77 | 7.62 | 0.0021 | 0.003 | 0.0041 |
| 3239927 | 0.6 | 0.05 | 1.16 | 0.59 | 0.8822 | 0.6 | 0.59 | 0.45 | 0.72 | 4.55 | 0.0439 | 0.0275 | 0.0453 |
| 3239982 | 0.64 | 0.0064 | 1.35 | 0.66 | 0.3262 | 0.6 | 0.7 | 0.53 | 0.75 | 5.78 | 0.0005 | 0.0045 | 0.0072 |
| 3240135 | 0.65 | 0.0041 | 1.38 | 0.64 | 0.5397 | 0.49 | 0.72 | 0.5 | 0.71 | 4.11 | 0.0108 | 0.0053 | 0.0075 |
| 3240409 | 0.7 | 0.0002 | 1.31 | 0.71 | 0.0583 | 0.68 | 0.72 | 0.58 | 0.8 | 7.67 | 0 | 0.0001 | 0.0002 |
| 3240425 | 0.67 | 0.0015 | 1.45 | 0.61 | 0.7405 | 0.7 | 0.56 | 0.48 | 0.77 | 6.55 | 0.0031 | 0.0028 | 0.0037 |
| 3241499 | 0.65 | 0.0063 | 1.46 | 0.64 | 0.5397 | 0.53 | 0.7 | 0.5 | 0.72 | 4.32 | 0.0036 | 0.004 | 0.0086 |
| 3241506 | 0.67 | 0.0017 | 1.32 | 0.6 | 0.7956 | 0.66 | 0.57 | 0.47 | 0.75 | 4.59 | 0.0085 | 0.0023 | 0.0038 |
| 3243292 | 0.65 | 0.004 | 1.46 | 0.67 | 0.2627 | 0.6 | 0.71 | 0.54 | 0.75 | 5.81 | 0.0003 | 0.0018 | 0.0029 |
| 3250201 | 0.63 | 0.012 | 1.62 | 0.62 | 0.6786 | 0.57 | 0.65 | 0.48 | 0.73 | 4.64 | 0.0073 | 0.0066 | 0.0083 |
| 3251409 | 0.68 | 0.0008 | 1.39 | 0.67 | 0.206 | 0.53 | 0.76 | 0.56 | 0.74 | 5.94 | 0.0003 | 0.0002 | 0.0011 |
| 3252608 | 0.63 | 0.0173 | 1.26 | 0.64 | 0.5397 | 0.55 | 0.68 | 0.5 | 0.73 | 4.94 | 0.0057 | 0.0122 | 0.0165 |
| 3253445 | 0.67 | 0.0016 | 1.32 | 0.64 | 0.5397 | 0.77 | 0.56 | 0.5 | 0.81 | 6.69 | 0.0007 | 0.0013 | 0.0022 |
| 3253863 | 0.65 | 0.0059 | 1.41 | 0.64 | 0.4669 | 0.55 | 0.7 | 0.51 | 0.73 | 5.07 | 0.0071 | 0.0015 | 0.0026 |
| 3255269 | 0.65 | 0.0037 | 1.35 | 0.67 | 0.2627 | 0.6 | 0.71 | 0.54 | 0.75 | 6.58 | 0.0005 | 0.0015 | 0.0025 |
| 3257404 | 0.64 | 0.0104 | 1.47 | 0.61 | 0.7405 | 0.47 | 0.7 | 0.47 | 0.7 | 4.78 | 0.0455 | 0.0629 | 0.0592 |
| 3258021 | 0.67 | 0.0011 | 1.5 | 0.65 | 0.3949 | 0.6 | 0.68 | 0.52 | 0.75 | 5.61 | 0.0019 | 0.0015 | 0.0017 |
| 3259678 | 0.61 | 0.0378 | 1.34 | 0.61 | 0.7405 | 0.45 | 0.71 | 0.47 | 0.69 | 5.5 | 0.0718 | 0.023 | 0.0354 |
| 3361550 | 0.58 | 0.1179 | 1.38 | 0.63 | 0.611 | 0.51 | 0.7 | 0.49 | 0.71 | 4.45 | 0.0181 | 0.1173 | 0.1286 |
| 3262470 | 0.63 | 0.0166 | 1.23 | 0.6 | 0.8429 | 0.55 | 0.62 | 0.46 | 0.71 | 5.11 | 0.0448 | 0.0149 | 0.0211 |
| 3263804 | 0.67 | 0.0017 | 1.25 | 0.64 | 0.4669 | 0.57 | 0.68 | 0.51 | 0.74 | 5.79 | 0.001 | 0.0013 | 0.0029 |
| 3264697 | 0.66 | 0.0033 | 1.37 | 0.67 | 0.2627 | 0.53 | 0.74 | 0.54 | 0.73 | 5.83 | 0.0009 | 0.0031 | 0.0046 |
| 3264726 | 0.63 | 0.0143 | 1.52 | 0.58 | 0.914 | 0.62 | 0.56 | 0.45 | 0.72 | 5.14 | 0.0323 | 0.01 | 0.0139 |
| 3275175 | 0.66 | 0.0035 | 1.37 | 0.64 | 0.4669 | 0.57 | 0.68 | 0.51 | 0.74 | 5.13 | 0.0045 | 0.0022 | 0.0035 |
| 3276455 | 0.63 | 0.0168 | 1.26 | 0.61 | 0.7405 | 0.43 | 0.72 | 0.47 | 0.69 | 3.86 | 0.0883 | 0.0323 | 0.0325 |
| 3277709 | 0.69 | 0.0003 | 1.36 | 0.71 | 0.0583 | 0.66 | 0.73 | 0.58 | 0.79 | 4.6 | 0 | 0.001 | 0.0018 |
| 3279936 | 0.65 | 0.0049 | 1.31 | 0.6 | 0.7956 | 0.72 | 0.54 | 0.47 | 0.77 | 7.18 | 0.0043 | 0.0036 | 0.0048 |
| 3280961 | 0.64 | 0.0076 | 1.37 | 0.63 | 0.611 | 0.49 | 0.71 | 0.49 | 0.71 | 4.34 | 0.0322 | 0.016 | 0.0113 |
| 3282022 | 0.61 | 0.0391 | 1.43 | 0.62 | 0.6786 | 0.62 | 0.62 | 0.48 | 0.74 | 4.4 | 0.0063 | 0.0275 | 0.037 |
| 3282170 | 0.64 | 0.0073 | 1.28 | 0.64 | 0.5397 | 0.55 | 0.68 | 0.5 | 0.73 | 3.64 | 0.0035 | 0.0091 | 0.0169 |
| 3282215 | 0.66 | 0.0021 | 1.64 | 0.64 | 0.5397 | 0.62 | 0.65 | 0.5 | 0.75 | 4.71 | 0.0032 | 0.0019 | 0.0026 |
| 3282220 | 0.69 | 0.0004 | 1.66 | 0.68 | 0.1571 | 0.6 | 0.73 | 0.56 | 0.76 | 5.4 | 0.0001 | 0.0003 | 0.0005 |
| 3282254 | 0.65 | 0.0052 | 1.32 | 0.64 | 0.5397 | 0.64 | 0.63 | 0.5 | 0.75 | 5.13 | 0.002 | 0.0035 | 0.0058 |
| 3284025 | 0.64 | 0.008 | 1.29 | 0.67 | 0.206 | 0.45 | 0.8 | 0.57 | 0.72 | 5.09 | 0.0011 | 0.0055 | 0.0091 |
| 3284217 | 0.67 | 0.0015 | 1.39 | 0.66 | 0.3262 | 0.74 | 0.61 | 0.52 | 0.81 | 6.6 | 0.0001 | 0.0006 | 0.0011 |
| 3284603 | 0.64 | 0.0096 | 1.31 | 0.63 | 0.611 | 0.57 | 0.66 | 0.49 | 0.73 | 5.47 | 0.008 | 0.0048 | 0.0083 |
| 3284665 | 0.65 | 0.0043 | 1.44 | 0.62 | 0.6786 | 0.57 | 0.65 | 0.48 | 0.73 | 5.98 | 0.005 | 0.0021 | 0.0049 |
| 3294341 | 0.64 | 0.0107 | 1.39 | 0.63 | 0.611 | 0.49 | 0.71 | 0.49 | 0.71 | 4.54 | 0.016 | 0.0053 | 0.007 |
| 3294501 | 0.63 | 0.0126 | 1.35 | 0.64 | 0.5397 | 0.55 | 0.68 | 0.5 | 0.73 | 4.92 | 0.0073 | 0.0084 | 0.0116 |
| 3298980 | 0.62 | 0.0278 | 1.32 | 0.66 | 0.3262 | 0.55 | 0.72 | 0.53 | 0.74 | 4.08 | 0.0028 | 0.0389 | 0.0492 |
| 3301222 | 0.61 | 0.0387 | 1.34 | 0.61 | 0.7405 | 0.72 | 0.55 | 0.48 | 0.78 | 6.14 | 0.0021 | 0.0264 | 0.078 |
| 3301703 | 0.65 | 0.0052 | 1.4 | 0.68 | 0.1571 | 0.49 | 0.79 | 0.58 | 0.73 | 3.9 | 0.0005 | 0.0028 | 0.0045 |
| 3301858 | 0.62 | 0.0213 | 1.32 | 0.64 | 0.4669 | 0.36 | 0.8 | 0.52 | 0.69 | 4.2 | 0.0276 | 0.027 | 0.0287 |
| 3301863 | 0.65 | 0.0059 | 1.43 | 0.67 | 0.2627 | 0.72 | 0.63 | 0.53 | 0.8 | 7.07 | 0.0001 | 0.0041 | 0.0042 |
| 3302047 | 0.67 | 0.0014 | 1.37 | 0.64 | 0.5397 | 0.66 | 0.62 | 0.5 | 0.76 | 4.62 | 0.003 | 0.0024 | 0.0045 |
| 3304013 | 0.64 | 0.0101 | 1.31 | 0.62 | 0.6786 | 0.68 | 0.59 | 0.48 | 0.76 | 6.32 | 0.0039 | 0.0079 | 0.0103 |
| 3304626 | 0.62 | 0.0271 | 1.18 | 0.61 | 0.7405 | 0.47 | 0.7 | 0.47 | 0.7 | 5.7 | 0.0312 | 0.0162 | 0.024 |
| 3304652 | 0.67 | 0.0016 | 1.37 | 0.64 | 0.5397 | 0.47 | 0.73 | 0.5 | 0.71 | 4.89 | 0.0119 | 0.0012 | 0.0034 |
| 3307940 | 0.64 | 0.0096 | 1.32 | 0.61 | 0.7405 | 0.51 | 0.67 | 0.4 | 0.71 | 3.94 | 0.0428 | 0.0066 | 0.0098 |
| 3308532 | 0.63 | 0.0175 | 1.24 | 0.6 | 0.8429 | 0.34 | 0.74 | 0.43 | 0.66 | 5.37 | 0.187 | 0.1171 | 0.1202 |
| 3309267 | 0.62 | 0.0189 | 1.35 | 0.6 | 0.8429 | 0.64 | 0.57 | 0.46 | 0.73 | 4.83 | 0.0118 | 0.01 | 0.0202 |
| 3311304 | 0.68 | 0.0009 | 1.38 | 0.66 | 0.3262 | 0.47 | 0.77 | 0.54 | 0.72 | 3.87 | 0.0086 | 0.0027 | 0.0029 |
| 3317557 | 0.68 | 0.0007 | 1.35 | 0.63 | 0.611 | 0.45 | 0.73 | 0.49 | 0.7 | 3.97 | 0.0466 | 0.0078 | 0.0066 |
| 3325714 | 0.63 | 0.0155 | 1.25 | 0.6 | 0.7956 | 0.49 | 0.67 | 0.46 | 0.7 | 4.26 | 0.0761 | 0.012 | 0.0164 |
| 3325963 | 0.64 | 0.0078 | 1.48 | 0.64 | 0.5397 | 0.68 | 0.61 | 0.5 | 0.77 | 5.56 | 0.0015 | 0.0087 | 0.0072 |
| 3326246 | 0.62 | 0.0224 | 1.33 | 0.63 | 0.611 | 0.4 | 0.76 | 0.49 | 0.69 | 4.11 | 0.055 | 0.0349 | 0.0402 |
| 3326465 | 0.7 | 0.0001 | 1.43 | 0.65 | 0.3949 | 0.55 | 0.71 | 0.52 | 0.73 | 3.43 | 0.0027 | 1 | 0.0001 |
| 3329941 | 0.64 | 0.0067 | 1.3 | 0.65 | 0.3949 | 0.6 | 0.68 | 0.52 | 0.75 | 5 | 0.0017 | 0.0066 | 0.0127 |
| 3331532 | 0.7 | 0.0002 | 1.36 | 0.65 | 0.3949 | 0.66 | 0.65 | 0.52 | 0.77 | 6.06 | 0.001 | 0.0003 | 0.0003 |
| 3331573 | 0.61 | 0.041 | 1.32 | 0.64 | 0.5397 | 0.64 | 0.63 | 0.5 | 0.75 | 5.05 | 0.0021 | 0.0152 | 0.0247 |
| 3331613 | 0.69 | 0.0005 | 1.4 | 0.66 | 0.3262 | 0.62 | 0.68 | 0.53 | 0.76 | 6.7 | 0.000 | 0.0005 | 0.0007 |
| 3331614 | 0.67 | 0.0018 | 1.44 | 0.63 | 0.611 | 0.77 | 0.55 | 0.49 | 0.8 | 6.86 | 0.0006 | 0.002 | 0.0023 |
| 3333687 | 0.62 | 0.0205 | 1.34 | 0.62 | 0.6786 | 0.6 | 0.63 | 0.48 | 0.73 | 5.33 | 0.0128 | 0.0251 | 0.0227 |
| 3334162 | 0.63 | 0.0164 | 1.32 | 0.64 | 0.5397 | 0.49 | 0.72 | 0.5 | 0.71 | 5.02 | 0.0131 | 0.0201 | 0.0302 |
| 3334515 | 0.66 | 0.0027 | 1.33 | 0.6 | 0.7956 | 0.72 | 0.54 | 0.47 | 0.77 | 7.19 | 0.0036 | 0.0021 | 0.0032 |
| 3335173 | 0.66 | 0.0022 | 1.61 | 0.64 | 0.5397 | 0.74 | 0.57 | 0.5 | 0.8 | 6.66 | 0.0005 | 0.0011 | 0.0012 |
| 3335176 | 0.64 | 0.0069 | 1.38 | 0.61 | 0.7405 | 0.64 | 0.6 | 0.48 | 0.74 | 6.61 | 0.0111 | 0.0053 | 0.0053 |
| 3335178 | 0.68 | 0.0006 | 1.38 | 0.64 | 0.4669 | 0.68 | 0.62 | 0.51 | 0.77 | 6.69 | 0.0005 | 0.0004 | 0.0006 |
| 3335179 | 0.67 | 0.0012 | 1.5 | 0.66 | 0.3262 | 0.66 | 0.66 | 0.53 | 0.77 | 5.65 | 0.0005 | 0.0009 | 0.0011 |
| 3335181 | 0.67 | 0.0014 | 1.34 | 0.6 | 0.8429 | 0.72 | 0.52 | 0.47 | 0.77 | 8.05 | 0.0062 | 0.0023 | 0.003 |
| 3335182 | 0.67 | 0.0014 | 1.31 | 0.64 | 0.5397 | 0.74 | 0.57 | 0.5 | 0.8 | 8.91 | 0.0006 | 0.0015 | 0.0023 |
| 3335187 | 0.67 | 0.0012 | 1.39 | 0.64 | 0.4669 | 0.72 | 0.6 | 0.51 | 0.79 | 8.46 | 0.0003 | 0.0013 | 0.0019 |
| 3335188 | 0.67 | 0.0015 | 1.29 | 0.64 | 0.4669 | 0.77 | 0.57 | 0.51 | 0.81 | 9.17 | 0.0003 | 0.0015 | 0.0019 |
| 3335189 | 0.63 | 0.0123 | 1.29 | 0.58 | 0.914 | 0.68 | 0.52 | 0.45 | 0.74 | 5.49 | 0.0282 | 0.0086 | 0.0146 |
| 3335191 | 0.66 | 0.0021 | 1.33 | 0.62 | 0.6786 | 0.7 | 0.57 | 0.49 | 0.77 | 8.73 | 0.0019 | 0.0013 | 0.0024 |
| 3335231 | 0.66 | 0.0027 | 1.45 | 0.61 | 0.7405 | 0.77 | 0.52 | 0.48 | 0.8 | 7.5 | 0.0017 | 0.0027 | 0.0031 |
| 3337724 | 0.68 | 0.0007 | 1.29 | 0.67 | 0.206 | 0.68 | 0.67 | 0.54 | 0.79 | 7.12 | 0.0001 | 0.0008 | 0.0009 |
| 3338618 | 0.67 | 0.0014 | 1.36 | 0.67 | 0.206 | 0.66 | 0.68 | 0.54 | 0.78 | 7.3 | 0.0001 | 0.001 | 0.0017 |
| 3341466 | 0.62 | 0.0275 | 1.23 | 0.61 | 0.7405 | 0.68 | 0.57 | 0.48 | 0.76 | 5.31 | 0.0056 | 0.0256 | 0.0366 |
| 3341504 | 0.67 | 0.0016 | 1.56 | 0.64 | 0.4669 | 0.66 | 0.63 | 0.51 | 0.76 | 5.61 | 0.0015 | 0.0008 | 0.0013 |
| 3344853 | 0.65 | 0.0044 | 1.68 | 0.63 | 0.611 | 0.62 | 0.63 | 0.49 | 0.74 | 5 | 0.0049 | 0.0031 | 0.0031 |
| 3344889 | 0.63 | 0.0162 | 1.44 | 0.62 | 0.6786 | 0.49 | 0.7 | 0.48 | 0.7 | 3.97 | 0.0201 | 0.0071 | 0.0143 |
| 3345453 | 0.6 | 0.0586 | 1.73 | 0.61 | 0.7405 | 0.55 | 0.65 | 0.47 | 0.72 | 5 | 0.0209 | 0.0264 | 0.0332 |
| 3347691 | 0.62 | 0.0255 | 1.31 | 0.6 | 0.7956 | 0.64 | 0.59 | 0.47 | 0.74 | 6 | 0.0102 | 0.0264 | 0.0231 |
| 3352533 | 0.68 | 0.0005 | 1.25 | 0.62 | 0.6786 | 0.7 | 0.57 | 0.49 | 0.77 | 5.44 | 0.0029 | 0.0006 | 0.0011 |
| 3352561 | 0.6 | 0.0292 | 1.25 | 0.64 | 0.5397 | 0.62 | 0.65 | 0.5 | 0.75 | 4.52 | 0.0069 | 0.0438 | 0.0471 |
| 3352585 | 0.63 | 0.0137 | 1.33 | 0.6 | 0.7956 | 0.74 | 0.52 | 0.47 | 0.78 | 5.69 | 0.0024 | 0.0081 | 0.0126 |
| 3352925 | 0.63 | 0.0147 | 1.33 | 0.64 | 0.5397 | 0.6 | 0.66 | 0.5 | 0.74 | 3.22 | 0.0034 | 0.0033 | 0.0069 |
| 3354966 | 0.65 | 0.0039 | 1.31 | 0.64 | 0.5397 | 0.68 | 0.61 | 0.5 | 0.77 | 7.27 | 0.0012 | 0.0031 | 0.004 |
| 3356173 | 0.63 | 0.0135 | 1.38 | 0.63 | 0.611 | 0.6 | 0.65 | 0.49 | 0.74 | 4.81 | 0.0066 | 0.0132 | 0.0137 |
| 3358364 | 0.66 | 0.0034 | 1.41 | 0.6 | 0.8429 | 0.72 | 0.52 | 0.47 | 0.77 | 5.54 | 0.0099 | 0.0036 | 0.0041 |
| 3359382 | 0.66 | 0.0029 | 1.4 | 0.66 | 0.3262 | 0.51 | 0.74 | 0.53 | 0.73 | 4.41 | 0.0012 | 0.0005 | 0.0021 |
| 3362739 | 0.65 | 0.0044 | 1.54 | 0.64 | 0.5397 | 0.6 | 0.66 | 0.5 | 0.74 | 4.54 | 0.0037 | 0.0028 | 0.0037 |
| 3362742 | 0.67 | 0.0014 | 1.61 | 0.64 | 0.5397 | 0.62 | 0.65 | 0.5 | 0.75 | 5.27 | 0.0029 | 0.0013 | 0.0021 |
| 3362745 | 0.62 | 0.0233 | 1.39 | 0.62 | 0.6786 | 0.6 | 0.63 | 0.48 | 0.73 | 5.25 | 0.005 | 0.0105 | 0.0174 |
| 3362763 | 0.66 | 0.0027 | 1.24 | 0.63 | 0.611 | 0.53 | 0.68 | 0.49 | 0.72 | 5.42 | 0.0192 | 0.0045 | 0.0045 |
| 3362773 | 0.67 | 0.0014 | 1.36 | 0.65 | 0.3949 | 0.62 | 0.67 | 0.52 | 0.75 | 7.19 | 0.001 | 0.0011 | 0.0012 |
| 3362943 | 0.65 | 0.0035 | 1.66 | 0.64 | 0.4669 | 0.62 | 0.66 | 0.51 | 0.75 | 4.96 | 0.0015 | 0.0023 | 0.003 |
| 3363519 | 0.62 | 0.0216 | 1.31 | 0.57 | 0.9388 | 0.62 | 0.55 | 0.44 | 0.71 | 4.13 | 0.0545 | 0.0285 | 0.0343 |
| 3363981 | 0.63 | 0.0113 | 1.31 | 0.64 | 0.4669 | 0.62 | 0.66 | 0.51 | 0.75 | 3.46 | 0.0023 | 0.0399 | 0.0533 |
| 3364729 | 0.6 | 0.0506 | 1.36 | 0.6 | 0.8429 | 0.53 | 0.63 | 0.45 | 0.7 | 4.33 | 0.0626 | 0.0548 | 0.0644 |
| 3364770 | 0.65 | 0.0036 | 1.41 | 0.64 | 0.5397 | 0.57 | 0.67 | 0.5 | 0.73 | 4.17 | 0.0077 | 0.0058 | 0.0055 |
| 3364800 | 0.63 | 0.0121 | 1.34 | 0.66 | 0.3262 | 0.57 | 0.71 | 0.53 | 0.74 | 3.63 | 0.0047 | 0.026 | 0.0109 |
| 3368920 | 0.65 | 0.0049 | 1.42 | 0.64 | 0.4669 | 0.62 | 0.66 | 0.51 | 0.75 | 5.77 | 0.0022 | 0.0049 | 0.0053 |
| 3371737 | 0.63 | 0.0113 | 1.28 | 0.64 | 0.5397 | 0.62 | 0.65 | 0.5 | 0.75 | 6.21 | 0.0021 | 0.0143 | 0.0175 |
| 3372461 | 0.66 | 0.0027 | 1.67 | 0.64 | 0.5397 | 0.51 | 0.71 | 0.5 | 0.72 | 3.75 | 0.0086 | 0.0055 | 0.0064 |
| 3375309 | 0.61 | 0.042 | 1.51 | 0.6 | 0.7956 | 0.64 | 0.59 | 0.47 | 0.74 | 4.89 | 0.0196 | 0.0433 | 0.0396 |
| 3375752 | 0.67 | 0.0019 | 1.32 | 0.64 | 0.5397 | 0.66 | 0.62 | 0.5 | 0.76 | 5.07 | 0.0017 | 0.0019 | 0.0029 |
| 3375863 | 0.65 | 0.0038 | 1.33 | 0.64 | 0.4669 | 0.57 | 0.68 | 0.51 | 0.74 | 4.77 | 0.0072 | 0.0039 | 0.0039 |
| 3376090 | 0.65 | 0.0036 | 1.39 | 0.64 | 0.4669 | 0.66 | 0.63 | 0.51 | 0.76 | 5.73 | 0.0012 | 0.0028 | 0.0039 |
| 3376775 | 0.65 | 0.004 | 1.26 | 0.58 | 0.914 | 0.68 | 0.52 | 0.45 | 0.74 | 5.44 | 0.0148 | 0.0042 | 0.0074 |
| 3377457 | 0.65 | 0.0044 | 1.7 | 0.64 | 0.5397 | 0.62 | 0.65 | 0.5 | 0.75 | 5.07 | 0.0025 | 0.0029 | 0.0042 |
| 3377464 | 0.65 | 0.0063 | 1.49 | 0.64 | 0.5397 | 0.64 | 0.63 | 0.5 | 0.75 | 6.16 | 0.0023 | 0.0035 | 0.0043 |
| 3377621 | 0.64 | 0.0064 | 1.64 | 0.61 | 0.7405 | 0.7 | 0.56 | 0.48 | 0.77 | 6.49 | 0.003 | 0.0037 | 0.0059 |
| 3377623 | 0.64 | 0.0103 | 1.48 | 0.6 | 0.8429 | 0.66 | 0.56 | 0.46 | 0.74 | 6.38 | 0.0113 | 0.0064 | 0.0097 |
| 3377625 | 0.65 | 0.0053 | 1.37 | 0.58 | 0.914 | 0.74 | 0.49 | 0.45 | 0.77 | 6.68 | 0.0104 | 0.0035 | 0.0045 |
| 3377632 | 0.64 | 0.0092 | 1.36 | 0.58 | 0.914 | 0.68 | 0.52 | 0.45 | 0.74 | 6.13 | 0.0192 | 0.0067 | 0.0092 |
| 3377633 | 0.61 | 0.0311 | 1.25 | 0.61 | 0.7405 | 0.53 | 0.66 | 0.47 | 0.71 | 6.84 | 0.0153 | 0.0164 | 0.0315 |
| 3377659 | 0.67 | 0.0013 | 1.3 | 0.63 | 0.611 | 0.7 | 0.59 | 0.49 | 0.77 | 9.07 | 0.0013 | 0.0008 | 0.001 |
| 3377660 | 0.66 | 0.0028 | 1.32 | 0.64 | 0.5397 | 0.7 | 0.6 | 0.5 | 0.78 | 7.75 | 0.0007 | 0.0022 | 0.0032 |
| 3379122 | 0.67 | 0.0013 | 1.52 | 0.63 | 0.611 | 0.72 | 0.57 | 0.49 | 0.78 | 6.2 | 0.0012 | 0.0013 | 0.0016 |
| 3379574 | 0.64 | 0.0079 | 1.44 | 0.65 | 0.3949 | 0.62 | 0.67 | 0.52 | 0.75 | 4.64 | 0.0006 | 0.0042 | 0.0073 |
| 3382981 | 0.65 | 0.0035 | 1.29 | 0.62 | 0.6786 | 0.62 | 0.62 | 0.48 | 0.74 | 5.05 | 0.0104 | 0.0046 | 0.006 |
| 3382984 | 0.67 | 0.0011 | 1.36 | 0.63 | 0.611 | 0.64 | 0.62 | 0.49 | 0.75 | 5.38 | 0.0025 | 0.001 | 0.0018 |
| 3383148 | 0.67 | 0.0011 | 1.47 | 0.63 | 0.611 | 0.72 | 0.57 | 0.49 | 0.78 | 6.79 | 0.0012 | 0.0015 | 0.0019 |
| 3383149 | 0.65 | 0.0046 | 1.57 | 0.62 | 0.6786 | 0.7 | 0.57 | 0.49 | 0.77 | 5.64 | 0.002 | 0.0022 | 0.0038 |
| 3384489 | 0.64 | 0.0064 | 1.53 | 0.64 | 0.4669 | 0.62 | 0.66 | 0.51 | 0.75 | 5.16 | 0.0021 | 0.0022 | 0.0038 |
| 3384495 | 0.65 | 0.0056 | 1.37 | 0.6 | 0.7956 | 0.72 | 0.54 | 0.47 | 0.77 | 4.1 | 0.0066 | 0.01 | 0.0102 |
| 3385045 | 0.65 | 0.0036 | 1.23 | 0.65 | 0.3949 | 0.45 | 0.77 | 0.53 | 0.71 | 3.67 | 0.0035 | 0.0018 | 0.0082 |
| 3385178 | 0.58 | 0.1362 | 1.29 | 0.6 | 0.7956 | 0.49 | 0.67 | 0.46 | 0.7 | 4.14 | 0.0565 | 0.1056 | 0.1188 |
| 3386816 | 0.65 | 0.0036 | 1.32 | 0.64 | 0.5397 | 0.55 | 0.68 | 0.5 | 0.73 | 7.23 | 0.0105 | 0.0051 | 0.0041 |
| 3387176 | 0.64 | 0.0104 | 1.29 | 0.64 | 0.5397 | 0.55 | 0.68 | 0.5 | 0.73 | 3.78 | 0.0099 | 0.0152 | 0.0211 |
| 3387136 | 0.66 | 0.0019 | 1.4 | 0.64 | 0.4669 | 0.62 | 0.66 | 0.51 | 0.75 | 5.79 | 0.0047 | 0.0057 | 0.0023 |
| 3388633 | 0.67 | 0.0015 | 1.37 | 0.67 | 0.206 | 0.64 | 0.7 | 0.55 | 0.77 | 5.23 | 0.0001 | 0.0018 | 0.002 |
| 3393789 | 0.67 | 0.0017 | 1.41 | 0.67 | 0.2627 | 0.62 | 0.7 | 0.54 | 0.76 | 5.4 | 0.0005 | 0.0019 | 0.0023 |
| 3394076 | 0.68 | 0.0008 | 1.31 | 0.69 | 0.1164 | 0.66 | 0.71 | 0.56 | 0.78 | 6.9 | 0 | 0.0007 | 0.0011 |
| 3394529 | 0.61 | 0.0323 | 1.32 | 0.63 | 0.611 | 0.6 | 0.65 | 0.49 | 0.74 | 4.77 | 0.0059 | 0.0226 | 0.032 |
| 3395420 | 0.65 | 0.0036 | 1.4 | 0.62 | 0.6786 | 0.68 | 0.59 | 0.48 | 0.76 | 6.13 | 0.0035 | 0.0025 | 0.0041 |
| 3395427 | 0.61 | 0.0323 | 1.25 | 0.64 | 0.5397 | 0.62 | 0.65 | 0.5 | 0.75 | 5.78 | 0.0021 | 0.0212 | 0.0335 |
| 3400152 | 0.64 | 0.0082 | 1.29 | 0.6 | 0.7956 | 0.6 | 0.61 | 0.47 | 0.72 | 4.87 | 0.0243 | 0.0097 | 0.0105 |
| 3402624 | 0.61 | 0.0323 | 1.25 | 0.61 | 0.7405 | 0.51 | 0.67 | 0.47 | 0.71 | 5.33 | 0.0387 | 0.03 | 0.0371 |
| 3402667 | 0.65 | 0.0059 | 1.25 | 0.6 | 0.7956 | 0.64 | 0.59 | 0.47 | 0.74 | 5.61 | 0.0086 | 0.0029 | 0.0051 |
| 3402720 | 0.64 | 0.0064 | 1.52 | 0.65 | 0.3949 | 0.64 | 0.66 | 0.52 | 0.76 | 5.58 | 0.0008 | 0.0034 | 0.0047 |
| 3406062 | 0.68 | 0.0007 | 1.35 | 0.71 | 0.0583 | 0.66 | 0.73 | 0.58 | 0.79 | 4.22 | 0 | 0.0008 | 0.0014 |
| 3407177 | 0.65 | 0.0061 | 1.27 | 0.61 | 0.7405 | 0.45 | 0.71 | 0.47 | 0.69 | 3.45 | 0.0396 | 0.003 | 0.0091 |
| 3407275 | 0.61 | 0.0445 | 1.28 | 0.57 | 0.9388 | 0.49 | 0.62 | 0.43 | 0.68 | 5.04 | 0.1771 | 0.0394 | 0.0555 |
| 3409292 | 0.63 | 0.0132 | 1.79 | 0.63 | 0.611 | 0.64 | 0.62 | 0.49 | 0.75 | 5.69 | 0.0028 | 0.0072 | 0.0183 |
| 3412651 | 0.65 | 0.0052 | 1.38 | 0.64 | 0.5397 | 0.55 | 0.68 | 0.5 | 0.73 | 5.41 | 0.0083 | 0.0059 | 0.0065 |
| 3414226 | 0.66 | 0.0027 | 1.47 | 0.68 | 0.1571 | 0.7 | 0.67 | 0.55 | 0.8 | 6.15 | 0 | 0.0011 | 0.0015 |
| 3416068 | 0.65 | 0.0049 | 1.28 | 0.62 | 0.6786 | 0.68 | 0.59 | 0.48 | 0.76 | 5.61 | 0.0025 | 0.0025 | 0.006 |
| 3416501 | 0.6 | 0.0668 | 1.26 | 0.64 | 0.5397 | 0.49 | 0.72 | 0.5 | 0.71 | 4.89 | 0.0185 | 0.0567 | 0.069 |
| 3417160 | 0.69 | 0.0004 | 1.49 | 0.64 | 0.4669 | 0.62 | 0.66 | 0.51 | 0.75 | 6.56 | 0.0023 | 0.0004 | 0.0006 |
| 3417666 | 0.69 | 0.0005 | 1.35 | 0.62 | 0.6786 | 0.62 | 0.62 | 0.48 | 0.74 | 5.97 | 0.0071 | 0.0007 | 0.0009 |
| 3418209 | 0.64 | 0.0066 | 1.4 | 0.62 | 0.6786 | 0.66 | 0.6 | 0.48 | 0.75 | 3.86 | 0.004 | 0.0086 | 0.0101 |
| 3419262 | 0.64 | 0.0066 | 1.3 | 0.64 | 0.4669 | 0.53 | 0.71 | 0.51 | 0.73 | 5.78 | 0.0052 | 0.0064 | 0.0087 |
| 3419622 | 0.61 | 0.0347 | 1.35 | 0.6 | 0.8429 | 0.6 | 0.6 | 0.46 | 0.72 | 4.81 | 0.0319 | 0.019 | 0.0313 |
| 3421154 | 0.64 | 0.009 | 1.42 | 0.59 | 0.8822 | 0.68 | 0.54 | 0.46 | 0.75 | 5.72 | 0.0154 | 0.0067 | 0.0081 |
| 3421223 | 0.64 | 0.0073 | 1.72 | 0.67 | 0.2627 | 0.55 | 0.73 | 0.54 | 0.74 | 5.06 | 0.001 | 0.0048 | 0.0044 |
| 3421350 | 1.65 | 0.006 | 1.42 | 0.61 | 0.7405 | 0.57 | 0.63 | 0.47 | 0.72 | 5.56 | 0.012 | 0.0031 | 0.0042 |
| 3421492 | 0.63 | 0.0182 | 1.31 | 0.66 | 0.3262 | 0.51 | 0.74 | 0.53 | 0.73 | 4.54 | 0.0027 | 0.0156 | 0.0146 |
| 3421652 | 0.64 | 0.0096 | 1.29 | 0.6 | 0.7956 | 0.53 | 0.65 | 0.46 | 0.71 | 6.73 | 0.027 | 0.0057 | 0.0071 |
| 3421668 | 0.67 | 0.0012 | 1.33 | 0.66 | 0.3262 | 0.47 | 0.77 | 0.54 | 0.77 | 4.65 | 0.0056 | 0.003 | 0.0027 |
| 3427811 | 0.63 | 0.0141 | 1.37 | 0.65 | 0.3949 | 0.53 | 0.72 | 0.52 | 0.73 | 3.88 | 0.0036 | 0.0185 | 0.0194 |
| 3427812 | 0.67 | 0.001 | 1.57 | 0.6 | 0.7956 | 0.62 | 0.6 | 0.47 | 0.73 | 5.18 | 0.0197 | 0.001 | 0.0013 |
| 3428711 | 0.66 | 0.0024 | 1.37 | 0.66 | 0.3262 | 0.45 | 0.78 | 0.54 | 0.71 | 4.49 | 0.0058 | 0.0029 | 0.004 |
| 3428712 | 0.65 | 0.0063 | 1.4 | 0.65 | 0.3949 | 0.47 | 0.76 | 0.52 | 0.71 | 4.45 | 0.0126 | 0.0096 | 0.0066 |
| 3429794 | 0.63 | 0.012 | 1.26 | 0.63 | 0.611 | 0.6 | 0.65 | 0.49 | 0.74 | 5.19 | 0.0079 | 0.0164 | 0.0221 |
| 3430575 | 0.62 | 0.0197 | 1.27 | 0.64 | 0.5397 | 0.57 | 0.67 | 0.5 | 0.73 | 6.41 | 0.0063 | 0.0155 | 0.0217 |
| 3431352 | 0.62 | 0.0205 | 1.32 | 0.62 | 0.6786 | 0.6 | 0.63 | 0.48 | 0.73 | 5.55 | 0.0128 | 0.0226 | 0.0185 |
| 3431686 | 0.66 | 0.0021 | 1.33 | 0.69 | 0.1164 | 0.62 | 0.73 | 0.57 | 0.77 | 5.51 | 0.0001 | 0.0029 | 0.003 |
| 3432171 | 0.66 | 0.0031 | 1.16 | 0.66 | 0.3262 | 0.66 | 0.66 | 0.53 | 0.77 | 5.85 | 0.0007 | 0.0016 | 0.0016 |
| 3432352 | 0.62 | 0.0282 | 1.29 | 0.6 | 0.7956 | 0.57 | 0.62 | 0.47 | 0.72 | 5.1 | 0.0177 | 0.018 | 0.0328 |
| 3435860 | 0.67 | 0.0011 | 1.51 | 0.64 | 0.4669 | 0.66 | 0.63 | 0.51 | 0.76 | 4.96 | 0.0007 | 0.0013 | 0.0025 |
| 3435864 | 0.66 | 0.0025 | 1.31 | 0.64 | 0.5397 | 0.7 | 0.6 | 0.5 | 0.78 | 5.62 | 0.0011 | 0.0011 | 0.0027 |
| 3436017 | 0.65 | 0.0036 | 1.33 | 0.64 | 0.5397 | 0.7 | 0.6 | 0.5 | 0.78 | 5.3 | 0.0015 | 0.0046 | 0.0056 |
| 3436264 | 0.66 | 0.0033 | 1.71 | 0.64 | 0.5397 | 0.64 | 0.63 | 0.5 | 0.75 | 3.99 | 0.0025 | 0.0022 | 0.003 |
| 3438044 | 0.63 | 0.0132 | 1.38 | 0.64 | 0.4669 | 0.49 | 0.73 | 0.51 | 0.71 | 4.94 | 0.0102 | 0.0134 | 0.0192 |
| 3445545 | 0.61 | 0.0319 | 1.31 | 0.64 | 0.5397 | 0.57 | 0.67 | 0.5 | 0.73 | 5.56 | 0.0025 | 0.0164 | 0.0259 |
| 3445674 | 0.67 | 0.0012 | 1.37 | 0.66 | 0.3262 | 0.45 | 0.78 | 0.54 | 0.71 | 4.05 | 0.0037 | 0.0133 | 0.0115 |
| 3445750 | 0.63 | 0.0126 | 1.34 | 0.65 | 0.3949 | 0.64 | 0.66 | 0.52 | 0.76 | 4.13 | 0.0012 | 0.0051 | 0.0066 |
| 3446488 | 0.64 | 0.0098 | 1.41 | 0.61 | 0.7405 | 0.64 | 0.6 | 0.48 | 0.74 | 5.7 | 0.007 | 0.0049 | 0.0083 |
| 3446869 | 0.66 | 0.0035 | 1.31 | 0.62 | 0.6786 | 0.7 | 0.57 | 0.49 | 0.77 | 7.73 | 0.0021 | 0.0021 | 0.0026 |
| 3446885 | 0.63 | 0.0112 | 1.3 | 0.6 | 0.8429 | 0.51 | 0.65 | 0.45 | 0.7 | 4.66 | 0.1091 | 0.0106 | 0.0108 |
| 3452256 | 0.64 | 0.0086 | 1.29 | 0.62 | 0.6786 | 0.68 | 0.59 | 0.48 | 0.76 | 5.45 | 0.0047 | 0.0083 | 0.0076 |
| 3452262 | 0.65 | 0.0039 | 1.49 | 0.64 | 0.4669 | 0.57 | 0.68 | 0.51 | 0.74 | 4.64 | 0.0051 | 0.0039 | 0.0044 |
| 3452324 | 0.63 | 0.0128 | 1.35 | 0.64 | 0.5397 | 0.6 | 0.66 | 0.5 | 0.74 | 5.58 | 0.0024 | 0.0102 | 0.0204 |
| 3452349 | 0.66 | 0.0028 | 1.18 | 0.64 | 0.4669 | 0.49 | 0.73 | 0.51 | 0.71 | 6.07 | 0.0064 | 0.0042 | 0.007 |
| 3454729 | 0.66 | 0.0032 | 1.29 | 0.64 | 0.5397 | 0.62 | 0.65 | 0.5 | 0.75 | 7.03 | 0.002 | 0.0019 | 0.0033 |
| 3456599 | 0.67 | 0.0017 | 1.52 | 0.63 | 0.611 | 0.55 | 0.67 | 0.49 | 0.72 | 4.38 | 0.0106 | 0.0011 | 0.0024 |
| 3457550 | 0.67 | 0.001 | 1.31 | 0.67 | 0.2627 | 0.57 | 0.72 | 0.54 | 0.75 | 7.1 | 0.0004 | 0.0006 | 0.001 |
| 3458494 | 0.65 | 0.0036 | 1.34 | 0.66 | 0.3262 | 0.62 | 0.68 | 0.53 | 0.76 | 5.37 | 0.0006 | 0.0016 | 0.0029 |
| 3461302 | 0.65 | 0.0036 | 1.44 | 0.63 | 0.611 | 0.64 | 0.62 | 0.49 | 0.75 | 6.12 | 0.0039 | 0.0042 | 0.0044 |
| 3461431 | 0.68 | 0.001 | 1.3 | 0.67 | 0.2627 | 0.66 | 0.67 | 0.53 | 0.77 | 3.87 | 0.0003 | 0.0002 | 0.0007 |
| 3462710 | 0.64 | 0.0109 | 1.31 | 0.63 | 0.611 | 0.6 | 0.65 | 0.49 | 0.74 | 6.28 | 0.0048 | 0.0092 | 0.0124 |
| 3462846 | 0.63 | 0.0115 | 1.35 | 0.61 | 0.7405 | 0.62 | 0.61 | 0.48 | 0.74 | 3.98 | 0.015 | 0.0156 | 0.0133 |
| 3462861 | 0.64 | 0.0065 | 1.33 | 0.63 | 0.611 | 0.68 | 0.6 | 0.49 | 0.77 | 4.84 | 0.0024 | 0.0101 | 0.0108 |
| 3462867 | 0.69 | 0.0004 | 1.35 | 0.62 | 0.6786 | 0.77 | 0.54 | 0.49 | 0.8 | 7.16 | 0.0011 | 0.0007 | 0.0008 |
| 3462868 | 0.67 | 0.0017 | 1.31 | 0.66 | 0.3262 | 0.51 | 0.74 | 0.53 | 0.73 | 4.64 | 0.0047 | 0.0059 | 0.0049 |
| 3462884 | 0.66 | 0.0031 | 1.32 | 0.66 | 0.3262 | 0.6 | 0.7 | 0.53 | 0.75 | 5.34 | 0.0008 | 0.0026 | 0.0031 |
| 3462988 | 0.67 | 0.0014 | 1.48 | 0.6 | 0.8429 | 0.66 | 0.56 | 0.46 | 0.74 | 5.48 | 0.0164 | 0.0015 | 0.0017 |
| 3463596 | 0.68 | 0.0008 | 1.29 | 0.67 | 0.206 | 0.51 | 0.77 | 0.56 | 0.73 | 6.43 | 0.0005 | 0.0006 | 0.0009 |
| 3463603 | 0.72 | 0 | 1.28 | 0.67 | 0.2627 | 0.36 | 0.84 | 0.57 | 0.7 | 4.81 | 0.0041 | 0.0001 | 0.0005 |
| 3464009 | 0.61 | 0.0405 | 1.33 | 0.62 | 0.6786 | 0.64 | 0.61 | 0.48 | 0.75 | 4.21 | 0.0069 | 0.0776 | 0.0787 |
| 3465251 | 0.66 | 0.0022 | 1.53 | 0.68 | 0.1571 | 0.66 | 0.7 | 0.55 | 0.78 | 4.84 | 0.0001 | 0.0045 | 0.0061 |
| 3465252 | 0.62 | 0.0261 | 1.62 | 0.65 | 0.3949 | 0.68 | 0.63 | 0.52 | 0.78 | 6.2 | 0.0006 | 0.0064 | 0.0095 |
| 3465278 | 0.63 | 0.0139 | 1.26 | 0.57 | 0.9388 | 0.26 | 0.76 | 0.38 | 0.64 | 5.12 | 0.7659 | 0.0836 | 0.1065 |
| 3466861 | 0.64 | 0.0109 | 1.34 | 0.65 | 0.3949 | 0.51 | 0.73 | 0.52 | 0.72 | 4.4 | 0.0021 | 0.0058 | 0.0164 |
| 3467642 | 0.64 | 0.0065 | 1.49 | 0.62 | 0.6786 | 0.62 | 0.62 | 0.48 | 0.74 | 5.63 | 0.0052 | 0.0061 | 0.0065 |
| 3468015 | 0.64 | 0.007 | 1.4 | 0.63 | 0.611 | 0.57 | 0.66 | 0.49 | 0.73 | 4.68 | 0.0087 | 0.0058 | 0.0106 |
| 3469369 | 0.64 | 0.0083 | 1.26 | 0.64 | 0.4669 | 0.4 | 0.78 | 0.51 | 0.7 | 3.35 | 0.042 | 0.0095 | 0.0114 |
| 3471177 | 0.64 | 0.0071 | 1.31 | 0.62 | 0.6786 | 0.49 | 0.7 | 0.48 | 0.7 | 4.56 | 0.0334 | 0.0041 | 0.0072 |
| 3471377 | 0.69 | 0.0004 | 1.69 | 0.69 | 0.1164 | 0.7 | 0.68 | 0.56 | 0.8 | 5.99 | 0 | 0.0001 | 0.0002 |
| 3471379 | 0.63 | 0.0182 | 1.31 | 0.61 | 0.7405 | 0.45 | 0.71 | 0.47 | 0.69 | 4.4 | 0.0397 | 0.0156 | 0.0335 |
| 3472092 | 0.64 | 0.0092 | 1.34 | 0.63 | 0.611 | 0.7 | 0.59 | 0.49 | 0.77 | 7.77 | 0.002 | 0.0052 | 0.0073 |
| 3473482 | 0.65 | 0.0045 | 1.33 | 0.63 | 0.611 | 0.49 | 0.71 | 0.49 | 0.71 | 4.29 | 0.0228 | 0.0043 | 0.0064 |
| 3474494 | 0.68 | 0.0008 | 1.36 | 0.66 | 0.3262 | 0.72 | 0.62 | 0.52 | 0.8 | 6.28 | 0.0003 | 0.0009 | 0.0012 |
| 3474937 | 0.62 | 0.0197 | 1.4 | 0.61 | 0.7405 | 0.55 | 0.65 | 0.47 | 0.72 | 5.34 | 0.0175 | 0.0218 | 0.0268 |
| 3475623 | 0.65 | 0.0059 | 1.29 | 0.64 | 0.4669 | 0.57 | 0.68 | 0.51 | 0.74 | 4.37 | 0.0023 | 0.0099 | 0.0158 |
| 3475737 | 0.64 | 0.0101 | 1.3 | 0.68 | 0.1571 | 0.57 | 0.74 | 0.56 | 0.75 | 4.9 | 0.0001 | 0.008 | 0.0139 |
| 3476294 | 0.65 | 0.0044 | 1.53 | 0.64 | 0.4669 | 0.68 | 0.62 | 0.51 | 0.77 | 5.56 | 0.0006 | 0.0026 | 0.0038 |
| 3480098 | 0.67 | 0.0017 | 1.57 | 0.64 | 0.5397 | 0.57 | 0.67 | 0.5 | 0.73 | 4.3 | 0.0023 | 0.0006 | 0.0014 |
| 3480100 | 0.67 | 0.0016 | 1.28 | 0.62 | 0.6786 | 0.49 | 0.7 | 0.48 | 0.7 | 3.34 | 0.0204 | 0.001 | 0.0033 |
| 3480173 | 0.66 | 0.0024 | 1.25 | 0.64 | 0.4669 | 0.47 | 0.74 | 0.51 | 0.71 | 5.63 | 0.0059 | 0.0029 | 0.0049 |
| 3480201 | 0.67 | 0.0014 | 1.4 | 0.64 | 0.4669 | 0.7 | 0.61 | 0.51 | 0.78 | 5.06 | 0.0005 | 0.0009 | 0.0018 |
| 3480203 | 0.65 | 0.0037 | 1.31 | 0.61 | 0.7405 | 0.66 | 0.59 | 0.48 | 0.75 | 5.79 | 0.0084 | 0.0036 | 0.0044 |
| 3480685 | 0.65 | 0.0042 | 1.3 | 0.64 | 0.4669 | 0.7 | 0.61 | 0.51 | 0.78 | 7.01 | 0.0005 | 0.0034 | 0.0054 |
| 3480856 | 0.7 | 0.0002 | 1.34 | 0.65 | 0.3949 | 0.6 | 0.68 | 0.52 | 0.75 | 6.74 | 0.0025 | 0.0002 | 0.0003 |
| 3481480 | 0.68 | 0.0005 | 1.37 | 0.66 | 0.3262 | 0.7 | 0.63 | 0.52 | 0.79 | 4.65 | 0.0003 | 0.0012 | 0.001 |
| 3482124 | 0.6 | 0.0729 | 1.32 | 0.6 | 0.7956 | 0.51 | 0.66 | 0.46 | 0.7 | 5.82 | 0.0443 | 0.0289 | 0.0537 |
| 3482915 | 0.65 | 0.0053 | 1.18 | 0.66 | 0.3262 | 0.55 | 0.72 | 0.53 | 0.74 | 4.94 | 0.0009 | 0.0086 | 0.0118 |
| 3482916 | 0.64 | 0.0073 | 1.62 | 0.68 | 0.1571 | 0.66 | 0.7 | 0.55 | 0.78 | 5.01 | 0 | 0.0021 | 0.0035 |
| 3483215 | 0.67 | 0.0015 | 1.28 | 0.65 | 0.3949 | 0.68 | 0.63 | 0.52 | 0.78 | 7.97 | 0.0003 | 0.0014 | 0.0022 |
| 3484743 | 0.67 | 0.0013 | 1.33 | 0.69 | 0.1164 | 0.68 | 0.7 | 0.56 | 0.79 | 6.76 | 0 | 0.0009 | 0.0015 |
| 3484750 | 0.66 | 0.0035 | 1.52 | 0.68 | 0.1571 | 0.7 | 0.67 | 0.55 | 0.8 | 4.99 | 0 | 0.002 | 0.0021 |
| 3484756 | 0.62 | 0.0248 | 1.32 | 0.61 | 0.7405 | 0.55 | 0.65 | 0.47 | 0.72 | 4.84 | 0.0186 | 0.0147 | 0.0182 |
| 3484762 | 0.68 | 0.0007 | 1.36 | 0.67 | 0.2627 | 0.62 | 0.7 | 0.54 | 0.76 | 5.98 | 0.0003 | 0.0008 | 0.0011 |
| 3485891 | 0.65 | 0.006 | 1.85 | 0.64 | 0.5397 | 0.6 | 0.66 | 0.5 | 0.74 | 4.72 | 0.0051 | 0.0036 | 0.0049 |
| 3489010 | 0.61 | 0.0331 | 1.43 | 0.59 | 0.8822 | 0.57 | 0.6 | 0.45 | 0.71 | 5.16 | 0.0525 | 0.0301 | 0.0337 |
| 3489011 | 0.66 | 0.0024 | 1.47 | 0.62 | 0.6786 | 0.77 | 0.54 | 0.49 | 0.8 | 7.21 | 0.001 | 0.0025 | 0.0028 |
| 3494193 | 0.63 | 0.017 | 1.45 | 0.61 | 0.7405 | 0.64 | 0.6 | 0.43 | 0.74 | 5.1 | 0.0042 | 0.0094 | 0.0158 |
| 3497656 | 0.59 | 0.0881 | 1.2 | 0.59 | 0.8822 | 0.49 | 0.65 | 0.44 | 0.69 | 3.62 | 0.1954 | 0.0862 | 0.0967 |
| 3497658 | 0.67 | 0.001 | 1.3 | 0.67 | 0.2627 | 0.66 | 0.67 | 0.53 | 0.77 | 6.29 | 0.0002 | 0.0011 | 0.0015 |
| 3497823 | 0.67 | 0.001 | 1.36 | 0.64 | 0.5397 | 0.6 | 0.66 | 0.5 | 0.74 | 4.65 | 0.0042 | 0.0028 | 0.0035 |
| 3498048 | 0.66 | 0.0019 | 1.4 | 0.61 | 0.7405 | 0.47 | 0.7 | 0.47 | 0.7 | 4.26 | 0.0419 | 0.0006 | 0.0016 |
| 3498445 | 0.61 | 0.0069 | 1.39 | 0.64 | 0.4669 | 0.64 | 0.65 | 0.51 | 0.76 | 5.51 | 0.0019 | 0.0031 | 0.0061 |
| 3498523 | 0.68 | 0.0007 | 1.74 | 0.66 | 0.3262 | 0.7 | 0.63 | 0.52 | 0.79 | 5.3 | 0.004 | 0.0009 | 0.0008 |
| 3498537 | 0.65 | 0.0039 | 1.33 | 0.64 | 0.4669 | 0.55 | 0.7 | 0.51 | 0.73 | 5.07 | 0.0031 | 0.0031 | 0.0051 |
| 3498977 | 0.67 | 0.0015 | 1.56 | 0.58 | 0.914 | 0.64 | 0.55 | 0.45 | 0.73 | 5.18 | 0.0316 | 0.0015 | 0.0018 |
| 3499545 | 0.65 | 0.0042 | 1.28 | 0.64 | 0.4669 | 0.62 | 0.66 | 0.51 | 0.75 | 5.43 | 0.0024 | 0.0036 | 0.0052 |
| 3500408 | 0.62 | 0.02 | 1.37 | 0.64 | 0.5397 | 0.57 | 0.67 | 0.5 | 0.73 | 5.1 | 0.0036 | 0.0089 | 0.0195 |
| 3501216 | 0.66 | 0.0022 | 1.32 | 0.64 | 0.5397 | 0.64 | 0.63 | 0.5 | 0.75 | 5.99 | 0.0024 | 0.0019 | 0.0039 |
| 3501217 | 0.67 | 0.0012 | 1.36 | 0.62 | 0.6786 | 0.7 | 0.57 | 0.49 | 0.77 | 6.19 | 0.0024 | 0.0011 | 0.0023 |
| 3501555 | 0.72 | 0 | 1.55 | 0.67 | 0.206 | 0.66 | 0.68 | 0.54 | 0.78 | 5.52 | 0.0001 | 0.0001 | 0.0001 |
| 3503211 | 0.66 | 0.0027 | 1.33 | 0.65 | 0.3949 | 0.6 | 0.68 | 0.52 | 0.75 | 6.44 | 0.001 | 0.0012 | 0.0024 |
| 3505453 | 0.63 | 0.0137 | 1.59 | 0.63 | 0.611 | 0.6 | 0.65 | 0.49 | 0.74 | 4.58 | 0.0162 | 0.0158 | 0.0132 |
| 3506941 | 0.63 | 0.0166 | 1.46 | 0.64 | 0.4669 | 0.62 | 0.66 | 0.51 | 0.75 | 4.86 | 0.0017 | 0.0083 | 0.0156 |
| 3508731 | 0.66 | 0.0024 | 1.39 | 0.66 | 0.3262 | 0.34 | 0.84 | 0.55 | 0.69 | 4.35 | 0.011 | 0.0067 | 0.0093 |
| 3508732 | 0.64 | 0.0084 | 1.3 | 0.67 | 0.206 | 0.64 | 0.7 | 0.55 | 0.77 | 5.5 | 0.0001 | 0.0057 | 0.0103 |
| 3508761 | 0.64 | 0.0106 | 1.28 | 0.62 | 0.6786 | 0.68 | 0.59 | 0.48 | 0.76 | 6.3 | 0.0033 | 0.0061 | 0.0115 |
| 3509912 | 0.66 | 0.0024 | 1.65 | 0.66 | 0.3262 | 0.6 | 0.7 | 0.53 | 0.75 | 4.21 | 0.0007 | 0.0032 | 0.0047 |
| 3509913 | 0.63 | 0.0126 | 1.35 | 0.64 | 0.4669 | 0.53 | 0.71 | 0.51 | 0.73 | 4 | 0.0057 | 0.0109 | 0.0169 |
| 3510070 | 0.64 | 0.0107 | 1.27 | 0.63 | 0.611 | 0.55 | 0.67 | 0.49 | 0.72 | 6.7 | 0.005 | 0.0039 | 0.0138 |
| 3510382 | 0.66 | 0.0028 | 1.43 | 0.63 | 0.611 | 0.62 | 0.63 | 0.49 | 0.74 | 5.54 | 0.0067 | 0.0024 | 0.0031 |
| 3512558 | 0.67 | 0.0018 | 1.36 | 0.6 | 0.8429 | 0.7 | 0.54 | 0.46 | 0.76 | 7.03 | 0.0064 | 0.0012 | 0.002 |
| 3513710 | 0.64 | 0.0086 | 1.51 | 0.64 | 0.5397 | 0.55 | 0.68 | 0.5 | 0.73 | 4.27 | 0.0066 | 0.0133 | 0.0103 |
| 3517677 | 0.68 | 0.0008 | 1.53 | 0.71 | 0.0583 | 0.4 | 0.88 | 0.66 | 0.72 | 4.2 | 0.0001 | 0.0001 | 0.0006 |
| 3517683 | 0.69 | 0.0005 | 1.28 | 0.7 | 0.0837 | 0.6 | 0.76 | 0.58 | 0.77 | 5.37 | 0.0001 | 0.0008 | 0.0025 |
| 3518501 | 0.64 | 0.0066 | 1.34 | 0.64 | 0.5397 | 0.49 | 0.72 | 0.5 | 0.71 | 4.95 | 0.0085 | 0.011 | 0.0121 |
| 3519136 | 0.66 | 0.0031 | 1.29 | 0.64 | 0.4669 | 0.49 | 0.73 | 0.51 | 0.71 | 4.81 | 0.0103 | 0.0083 | 0.0112 |
| 3521962 | 0.62 | 0.0236 | 1.39 | 0.67 | 0.2627 | 0.51 | 0.76 | 0.55 | 0.73 | 5.23 | 0.0005 | 0.0157 | 0.0241 |
| 3524677 | 0.66 | 0.0033 | 1.32 | 0.62 | 0.6786 | 0.66 | 0.6 | 0.48 | 0.75 | 5.38 | 0.0027 | 0.0041 | 0.0065 |
| 3524630 | 0.61 | 0.0369 | 1.46 | 0.64 | 0.5397 | 0.53 | 0.7 | 0.5 | 0.72 | 4.55 | 0.008 | 0.0212 | 0.0347 |
| 3524631 | 0.63 | 0.0112 | 1.39 | 0.6 | 0.7956 | 0.64 | 0.59 | 0.47 | 0.74 | 4.7 | 0.0066 | 0.0065 | 0.0108 |
| 3525314 | 0.68 | 0.0008 | 1.47 | 0.64 | 0.5397 | 0.64 | 0.63 | 0.5 | 0.75 | 6.24 | 0.002 | 0.0007 | 0.0012 |
| 3525317 | 0.69 | 0.0003 | 1.62 | 0.64 | 0.4669 | 0.62 | 0.66 | 0.51 | 0.75 | 5.31 | 0.0025 | 0.0002 | 0.0004 |
| 3525320 | 0.67 | 0.0013 | 1.48 | 0.62 | 0.6786 | 0.64 | 0.61 | 0.48 | 0.75 | 5.79 | 0.0045 | 0.0008 | 0.0019 |
| 3525547 | 0.66 | 0.003 | 1.35 | 0.61 | 0.7405 | 0.74 | 0.54 | 0.48 | 0.79 | 5.55 | 0.0032 | 0.0028 | 0.0032 |
| 3525702 | 0.65 | 0.0036 | 1.35 | 0.65 | 0.3949 | 0.53 | 0.72 | 0.52 | 0.73 | 5.08 | 0.0037 | 0.0038 | 0.0041 |
| 3525704 | 0.66 | 0.0028 | 1.42 | 0.59 | 0.8822 | 0.66 | 0.55 | 0.46 | 0.74 | 4.7 | 0.0253 | 0.0025 | 0.0031 |
| 3525710 | 0.68 | 0.001 | 1.32 | 0.66 | 0.3262 | 0.68 | 0.65 | 0.52 | 0.78 | 7.86 | 0.0002 | 0.0005 | 0.0007 |
| 3526383 | 0.68 | 0.0005 | 1.35 | 0.67 | 0.2627 | 0.49 | 0.77 | 0.55 | 0.72 | 3.05 | 0.0011 | 0.004 | 0.0086 |
| 3527530 | 0.67 | 0.0011 | 1.46 | 0.64 | 0.5397 | 0.68 | 0.61 | 0.5 | 0.77 | 4.61 | 0.0018 | 0.0011 | 0.0014 |
| 3528208 | 0.63 | 0.0145 | 1.52 | 0.64 | 0.4669 | 0.6 | 0.67 | 0.51 | 0.74 | 4.78 | 0.0028 | 0.0168 | 0.0138 |
| 3529094 | 0.63 | 0.0166 | 1.29 | 0.61 | 0.7405 | 0.64 | 0.6 | 0.48 | 0.74 | 4.76 | 0.0114 | 0.0186 | 0.0247 |
| 3531094 | 0.65 | 0.0063 | 1.45 | 0.6 | 0.7956 | 0.53 | 0.65 | 0.46 | 0.71 | 5.15 | 0.0372 | 0.0062 | 0.0107 |
| 3531491 | 0.72 | 0 | 1.61 | 0.69 | 0.1164 | 0.53 | 0.78 | 0.58 | 0.74 | 4.24 | 0.0001 | 0 | 0 |
| 3533452 | 0.68 | 0.0009 | 1.43 | 0.65 | 0.3949 | 0.74 | 0.6 | 0.51 | 0.8 | 6.12 | 0.0002 | 0.0007 | 0.0011 |
| 3534950 | 0.67 | 0.0011 | 1.4 | 0.68 | 0.1571 | 0.64 | 0.71 | 0.56 | 0.77 | 6.09 | 0.0001 | 0.0004 | 0.0011 |
| 3535917 | 0.66 | 0.0032 | 1.36 | 0.63 | 0.611 | 0.53 | 0.68 | 0.49 | 0.72 | 4.82 | 0.013 | 0.0044 | 0.0046 |
| 3536669 | 0.7 | 0.0001 | 1.28 | 0.71 | 0.0394 | 0.55 | 0.8 | 0.62 | 0.76 | 6.23 | 0 | 0.0001 | 0.0003 |
| 3536745 | 0.65 | 0.0056 | 1.46 | 0.64 | 0.5397 | 0.53 | 0.7 | 0.5 | 0.72 | 4.96 | 0.0138 | 0.0079 | 0.0077 |
| 3536931 | 0.65 | 0.006 | 1.34 | 0.63 | 0.611 | 0.64 | 0.62 | 0.49 | 0.75 | 7.03 | 0.003 | 0.0022 | 0.043 |
| 3536934 | 0.65 | 0.0036 | 1.33 | 0.64 | 0.5397 | 0.6 | 0.66 | 0.5 | 0.74 | 4.85 | 0.0026 | 0.0037 | 0.0064 |
| 3536938 | 0.61 | 0.0445 | 1.42 | 0.61 | 0.7405 | 0.6 | 0.62 | 0.47 | 0.73 | 5.85 | 0.0138 | 0.0162 | 0.0338 |
| 3536943 | 0.68 | 0.0005 | 1.33 | 0.65 | 0.3949 | 0.64 | 0.66 | 0.52 | 0.76 | 5.95 | 0.0005 | 0.0002 | 0.0006 |
| 3536948 | 0.64 | 0.0074 | 1.29 | 0.64 | 0.5397 | 0.62 | 0.65 | 0.5 | 0.75 | 5.94 | 0.0027 | 0.0033 | 0.0087 |
| 3536949 | 0.63 | 0.0151 | 1.57 | 0.62 | 0.6786 | 0.64 | 0.61 | 0.48 | 0.75 | 5.86 | 0.0063 | 0.0045 | 0.0082 |
| 3536951 | 0.65 | 0.0044 | 1.78 | 0.65 | 0.3949 | 0.66 | 0.65 | 0.52 | 0.77 | 4.39 | 0.0008 | 0.0022 | 0.0042 |
| 3536992 | 0.64 | 0.0074 | 1.39 | 0.64 | 0.5397 | 0.6 | 0.66 | 0.5 | 0.74 | 3.75 | 0.0062 | 0.0051 | 0.009 |
| 3536994 | 0.67 | 0.0013 | 1.37 | 0.64 | 0.5397 | 0.7 | 0.6 | 0.5 | 0.78 | 5.98 | 0.0015 | 0.0007 | 0.0012 |
| 3537787 | 0.67 | 0.0012 | 1.39 | 0.69 | 0.1164 | 0.55 | 0.77 | 0.58 | 0.75 | 5.2 | 0.0001 | 0.0005 | 0.0012 |
| 3537795 | 0.63 | 0.0159 | 1.68 | 0.63 | 0.611 | 0.64 | 0.62 | 0.49 | 0.75 | 5.68 | 0.0041 | 0.0085 | 0.0098 |
| 3537796 | 0.65 | 0.0054 | 1.94 | 0.6 | 0.8429 | 0.64 | 0.57 | 0.46 | 0.73 | 4.78 | 0.0173 | 0.0058 | 0.005 |
| 3539104 | 0.65 | 0.0061 | 1.35 | 0.61 | 0.7405 | 0.57 | 0.63 | 0.47 | 0.72 | 6.36 | 0.0133 | 0.0029 | 0.0051 |
| 3539877 | 0.06 | 0.0029 | 1.39 | 0.64 | 0.4669 | 0.53 | 0.71 | 0.51 | 0.73 | 4.25 | 0.0135 | 0.0097 | 0.0071 |
| 3543929 | 0.67 | 0.0017 | 1.36 | 0.6 | 0.7956 | 0.74 | 0.52 | 0.47 | 0.78 | 6.03 | 0.004 | 0.0017 | 0.0019 |
| 3544473 | 0.67 | 0.0014 | 1.4 | 0.65 | 0.3949 | 0.66 | 0.65 | 0.52 | 0.77 | 6.41 | 0.0005 | 0.0009 | 0.0015 |
| 3544890 | 0.62 | 0.0192 | 1.55 | 0.62 | 0.6786 | 0.45 | 0.72 | 0.48 | 0.69 | 4.77 | 0.0404 | 0.0098 | 0.0177 |
| 3545534 | 0.63 | 0.0115 | 1.4 | 0.64 | 0.5397 | 0.68 | 0.61 | 0.5 | 0.77 | 6.28 | 0.0015 | 0.0067 | 0.0096 |
| 3549043 | 0.63 | 0.0175 | 1.31 | 0.61 | 0.7405 | 0.55 | 0.65 | 0.47 | 0.72 | 5.16 | 0.0193 | 0.0161 | 0.0244 |
| 3550451 | 0.65 | 0.0045 | 1.43 | 0.63 | 0.611 | 0.62 | 0.63 | 0.49 | 0.74 | 5.73 | 0.0037 | 0.0017 | 0.0043 |
| 3550453 | 0.64 | 0.0094 | 1.29 | 0.65 | 0.3949 | 0.62 | 0.67 | 0.52 | 0.75 | 5.01 | 0.0018 | 0.0057 | 0.0108 |
| 3550455 | 0.63 | 0.0166 | 1.3 | 0.63 | 0.611 | 0.43 | 0.74 | 0.49 | 0.69 | 3.85 | 0.0424 | 0.0145 | 0.0172 |
| 3552937 | 0.63 | 0.0157 | 1.32 | 0.59 | 0.8822 | 0.53 | 0.62 | 0.45 | 0.7 | 4.68 | 0.0633 | 0.0091 | 0.0235 |
| 3552938 | 0.62 | 0.0236 | 1.39 | 0.58 | 0.914 | 0.53 | 0.61 | 0.44 | 0.69 | 5.07 | 0.0903 | 0.0166 | 0.0315 |
| 3556198 | 0.66 | 0.0032 | 1.33 | 0.65 | 0.3949 | 0.62 | 0.67 | 0.52 | 0.75 | 6.91 | 0.0007 | 0.002 | 0.0027 |
| 3556224 | 0.71 | 0.0001 | 1.35 | 0.66 | 0.3262 | 0.68 | 0.65 | 0.52 | 0.78 | 5.81 | 0.0004 | 0.0002 | 0.0002 |
| 3556278 | 0.68 | 0.0006 | 1.5 | 0.7 | 0.0837 | 0.53 | 0.79 | 0.6 | 0.75 | 4.27 | 0.0001 | 0.0002 | 0.0005 |
| 3556420 | 0.65 | 0.0044 | 1.44 | 0.65 | 0.3949 | 0.62 | 0.67 | 0.52 | 0.75 | 4.13 | 0.0013 | 0.0039 | 0.0049 |
| 3558292 | 0.65 | 0.006 | 1.37 | 0.62 | 0.6786 | 0.68 | 0.59 | 0.48 | 0.76 | 6.74 | 0.0039 | 0.0079 | 0.0089 |
| 3559519 | 0.67 | 0.0017 | 1.43 | 0.66 | 0.3262 | 0.45 | 0.78 | 0.54 | 0.71 | 4.32 | 0.0031 | 0.0009 | 0.0024 |
| 3559556 | 0.64 | 0.0097 | 1.38 | 0.61 | 0.5397 | 0.51 | 0.71 | 0.5 | 0.72 | 4.2 | 0.0092 | 0.0063 | 0.016 |
| 3562436 | 0.74 | 0 | 1.31 | 0.71 | 0.0583 | 0.47 | 0.84 | 0.63 | 0.73 | 3.52 | 1 | 0 | 0.0003 |
| 3563349 | 0.67 | 0.0014 | 1.27 | 0.67 | 0.206 | 0.6 | 0.72 | 0.55 | 0.76 | 4.86 | 0.0001 | 0.0024 | 0.0057 |
| 3563970 | 0.67 | 0.0015 | 1.52 | 0.64 | 0.4669 | 0.51 | 0.72 | 0.51 | 0.72 | 3.97 | 0.0056 | 0.001 | 0.002 |
| 3566596 | 0.65 | 0.0041 | 1.51 | 0.65 | 0.3949 | 0.68 | 0.63 | 0.52 | 0.78 | 6.11 | 0.0005 | 0.0017 | 0.0022 |
| 3566603 | 0.62 | 0.0197 | 1.43 | 0.61 | 0.7405 | 0.68 | 0.57 | 0.48 | 0.76 | 5.61 | 0.0052 | 0.0159 | 0.0172 |
| 3566648 | 0.64 | 0.0088 | 1.26 | 0.64 | 0.5397 | 0.47 | 0.73 | 0.5 | 0.71 | 4.44 | 0.0169 | 0.0083 | 0.0154 |
| 3566657 | 0.63 | 0.0137 | 1.18 | 0.64 | 0.4669 | 0.45 | 0.76 | 0.51 | 0.7 | 4.83 | 0.0054 | 0.0052 | 0.0162 |
| 3566993 | 0.63 | 0.0159 | 1.29 | 0.57 | 0.9388 | 0.66 | 0.52 | 0.44 | 0.73 | 4.8 | 0.0436 | 0.014 | 0.022 |
| 3569346 | 0.7 | 0.0001 | 1.38 | 0.67 | 0.206 | 0.51 | 0.77 | 0.56 | 0.73 | 5.02 | 0.0006 | 0.0002 | 0.0006 |
| 3169757 | 0.69 | 0.0003 | 1.46 | 0.68 | 0.1571 | 0.64 | 0.71 | 0.56 | 0.77 | 6.3 | 0.0001 | 0.0004 | 0.0005 |
| 3569759 | 0.66 | 0.0035 | 1.33 | 0.64 | 0.4669 | 0.64 | 0.65 | 0.51 | 0.76 | 5.73 | 0.0017 | 0.0027 | 0.0045 |
| 3569819 | 0.68 | 0.001 | 1.4 | 0.64 | 0.5397 | 0.66 | 0.62 | 0.5 | 0.76 | 4.92 | 0.0072 | 0.0025 | 0.0041 |
| 3570456 | 0.63 | 0.0145 | 1.32 | 0.58 | 0.914 | 0.66 | 0.54 | 0.45 | 0.73 | 5.49 | 0.0306 | 0.011 | 0.0165 |
| 3573155 | 0.63 | 0.0164 | 1.22 | 0.61 | 0.7405 | 0.47 | 0.7 | 0.47 | 0.7 | 5.19 | 0.0588 | 0.0201 | 0.0213 |
| 3576551 | 0.64 | 0.0096 | 1.31 | 0.6 | 0.8429 | 0.7 | 0.54 | 0.46 | 0.76 | 5.7 | 0.012 | 0.009 | 0.0098 |
| 3576567 | 0.67 | 0.0017 | 1.65 | 0.66 | 0.3262 | 0.55 | 0.72 | 0.53 | 0.74 | 4.59 | 0.0005 | 0.0006 | 0.0018 |
| 3576712 | 0.61 | 0.0445 | 1.31 | 0.6 | 0.7956 | 0.47 | 0.68 | 0.46 | 0.69 | 4.85 | 0.0503 | 0.0279 | 0.043 |
| 3576730 | 0.61 | 0.0364 | 1.55 | 0.64 | 0.5397 | 0.57 | 0.67 | 0.5 | 0.73 | 5.54 | 0.005 | 0.0107 | 0.0157 |
| 3576939 | 0.67 | 0.0018 | 1.51 | 0.61 | 0.7405 | 0.74 | 0.54 | 0.48 | 0.79 | 5.89 | 0.0016 | 0.0011 | 0.0012 |
| 3576940 | 0.67 | 0.0018 | 1.45 | 0.63 | 0.611 | 0.66 | 0.61 | 0.49 | 0.76 | 5.57 | 0.0032 | 0.0019 | 0.0028 |
| 3577874 | 0.69 | 0.0004 | 1.49 | 0.63 | 0.611 | 0.62 | 0.63 | 0.49 | 0.74 | 4.35 | 0.0064 | 0.0006 | 0.0007 |
| 3577898 | 0.64 | 0.01 | 1.43 | 0.66 | 0.3262 | 0.68 | 0.65 | 0.52 | 0.78 | 5.31 | 0.0003 | 0.0046 | 0.0062 |
| 3577911 | 0.69 | 0.0003 | 1.35 | 0.7 | 0.0837 | 0.62 | 0.74 | 0.58 | 0.77 | 5.07 | 0 | 0.0003 | 0.0013 |
| 3578431 | 0.68 | 0.0009 | 1.29 | 0.66 | 0.3262 | 0.49 | 0.76 | 0.53 | 0.72 | 5.19 | 0.0039 | 0.0016 | 0.0028 |
| 3580188 | 0.61 | 0.0335 | 1.25 | 0.62 | 0.6786 | 0.6 | 0.63 | 0.48 | 0.73 | 5.04 | 0.0065 | 0.0225 | 0.043 |
| 3580204 | 0.64 | 0.008 | 1.33 | 0.67 | 0.2627 | 0.6 | 0.71 | 0f1 | 0.75 | 6.87 | 0.0002 | 0.0045 | 0.0068 |
| 3580587 | 0.6 | 0.0535 | 1.22 | 0.63 | 0.611 | 0.45 | 0.73 | 0.49 | 0.7 | 3.6 | 0.0213 | 0.0342 | 0.0667 |
| 3580953 | 0.67 | 0.0017 | 1.3 | 0.57 | 0.9388 | 0.74 | 0.48 | 0.45 | 0.76 | 7.37 | 0.0157 | 0.0046 | 0.0061 |
| 3589660 | 0.63 | 0.0162 | 1.48 | 0.64 | 0.5397 | 0.55 | 0.68 | 0.5 | 0.73 | 4.41 | 0.0073 | 0.0165 | 0.0266 |
| 3590159 | 0.63 | 0.013 | 1.21 | 0.58 | 0.914 | 0.57 | 0.59 | 0.44 | 0.71 | 5.72 | 0.0615 | 0.0099 | 0.014 |
| 3590352 | 0.6 | 0.0729 | 1.26 | 0.57 | 0.9577 | 0.45 | 0.63 | 0.41 | 0.67 | 5.22 | 0.4343 | 0.1028 | 0.118 |
| 3591851 | 0.66 | 0.002 | 1.31 | 0.64 | 0.5397 | 0.62 | 0.65 | 0.5 | 0.75 | 4.89 | 0.0035 | 0.0008 | 0.0021 |
| 3591858 | 0.7 | 0.0001 | 1.31 | 0.65 | 0.3949 | 0.68 | 0.63 | 0.52 | 0.78 | 7.12 | 0.0004 | 0.0001 | 0.0002 |
| 3591985 | 0.64 | 0.007 | 1.38 | 0.65 | 0.3949 | 0.66 | 0.65 | 0.52 | 0.77 | 5.29 | 0.0005 | 0.0064 | 0.0105 |
| 3592046 | 0.62 | 0.0245 | 1.38 | 0.64 | 0.5397 | 0.66 | 0.62 | 0.5 | 0.76 | 5.86 | 0.0025 | 0.0244 | 0.0217 |
| 3592047 | 0.66 | 0.0026 | 1.34 | 0.63 | 0.611 | 0.7 | 0.59 | 0.49 | 0.77 | 6.72 | 0.0117 | 0.0047 | 0.003 |
| 3592395 | 0.66 | 0.0033 | 1.34 | 0.6 | 0.7956 | 0.43 | 0.71 | 0.45 | 0.68 | 4.01 | 0.0895 | 0.0047 | 0.0109 |
| 3593171 | 0.63 | 0.0182 | 1.76 | 0.64 | 0.5397 | 0.62 | 0.65 | 0.5 | 0.75 | 5.31 | 0.0025 | 0.0067 | 0.0081 |
| 3594101 | 0.63 | 0.0164 | 1.38 | 0.67 | 0.206 | 0.53 | 0.76 | 0.56 | 0.74 | 4.51 | 0.0005 | 0.0316 | 0.0396 |
| 3595811 | 0.61 | 0.0307 | 1.26 | 0.61 | 0.7405 | 0.64 | 0.6 | 0.48 | 0.74 | 4.86 | 0.003 | 0.0156 | 0.0357 |
| 3595872 | 0.64 | 0.0094 | 1.5 | 0.61 | 0.7405 | 0.64 | 0.6 | 0.48 | 0.74 | 5.15 | 0.0047 | 0.0058 | 0.0088 |
| 3596235 | 0.67 | 0.0012 | 1.27 | 0.66 | 0.3262 | 0.68 | 0.65 | 0.52 | 0.78 | 3.4 | 0.0003 | 0.0026 | 0.0052 |
| 3597406 | 0.64 | 0.0098 | 1.34 | 0.63 | 0.611 | 0.66 | 0.61 | 0.49 | 0.76 | 6.07 | 0.0025 | 0.0037 | 0.0067 |
| 3597468 | 0.63 | 0.0118 | 1.29 | 0.6 | 0.7956 | 0.7 | 0.55 | 0.47 | 0.76 | 5.92 | 0.0027 | 0.0057 | 0.0115 |
| 3597671 | 0.67 | 0.0018 | 1.29 | 0.6 | 0.7956 | 0.55 | 0.63 | 0.46 | 0.71 | 5.39 | 0.0257 | 0.0006 | 0.0016 |
| 3598717 | 0.65 | 0.0054 | 1.37 | 0.6 | 0.7956 | 0.68 | 0.56 | 0.47 | 0.75 | 6.37 | 0.0085 | 0.0035 | 0.0043 |
| 3599449 | 0.64 | 0.0078 | 1.24 | 0.61 | 0.7405 | 0.45 | 0.71 | 0.47 | 0.69 | 4.69 | 0.0736 | 0.0165 | 0.0195 |
| 3602509 | 0.67 | 0.0012 | 1.32 | 0.61 | 0.7405 | 0.68 | 0.57 | 0.48 | 0.76 | 5.32 | 0.0057 | 0.0012 | 0.0022 |
| 3605249 | 0.65 | 0.0046 | 1.32 | 0.62 | 0.6786 | 0.7 | 0.57 | 0.49 | 0.77 | 5.35 | 0.0032 | 0.0048 | 0.005 |
| 3606117 | 0.55 | 0.3487 | 1.1 | 0.64 | 0.5397 | 0.45 | 0.74 | 0.5 | 0.7 | 4.66 | 0.0257 | 0.1812 | 0.2086 |
| 3606409 | 0.68 | 0.0008 | 1.67 | 0.67 | 0.206 | 0.72 | 0.65 | 0.54 | 0.8 | 5.41 | 0 | 0.0005 | 0.0009 |
| 3608201 | 0.63 | 0.0121 | 1.29 | 0.63 | 0.611 | 0.57 | 0.66 | 0.49 | 0.73 | 4.98 | 0.0065 | 0.012 | 0.0187 |
| 3609166 | 0.65 | 0.0054 | 1.43 | 0.64 | 0.4669 | 0.66 | 0.63 | 0.51 | 0.76 | 4.97 | 0.0009 | 0.0042 | 0.0054 |
| 3615597 | 0.63 | 0.0132 | 1.22 | 0.64 | 0.4669 | 0.6 | 0.67 | 0.51 | 0.74 | 4.79 | 0.0036 | 0.0201 | 0.0207 |
| 3615604 | 0.64 | 0.0096 | 1.26 | 0.67 | 0.2627 | 0.51 | 0.76 | 0.55 | 0.73 | 6.32 | 0.0019 | 0.0139 | 0.0115 |
| 3615659 | 0.64 | 0.0085 | 1.24 | 0.62 | 0.6786 | 0.57 | 0.65 | 0.48 | 0.73 | 4.57 | 0.0171 | 0.0098 | 0.0126 |
| 3615989 | 0.67 | 0.0017 | 1.33 | 0.66 | 0.3262 | 0.64 | 0.67 | 0.53 | 0.76 | 4.74 | 0.0007 | 0.0006 | 0.0013 |
| 3621014 | 0.61 | 0.0082 | 1.35 | 0.66 | 0.3262 | 0.49 | 0.76 | 0.53 | 0.72 | 5.5 | 0.0024 | 0.0039 | 0.0073 |
| 3623329 | 0.63 | 0.0145 | 1.44 | 0.67 | 0.206 | 0.6 | 0.72 | 0.55 | 0.76 | 5.65 | 0.0002 | 0.0028 | 0.0062 |
| 3624199 | 0.67 | 0.0017 | 1.26 | 0.64 | 0.5397 | 0.6 | 0.66 | 0.5 | 0.74 | 6 | 0.0079 | 0.006 | 0.0048 |
| 3624533 | 0.65 | 0.0043 | 1.34 | 0.66 | 0.3262 | 0.57 | 0.71 | 0.53 | 0.74 | 4.7 | 0.0013 | 0.002 | 0.0055 |
| 3624702 | 0.63 | 0.0112 | 1.41 | 0.64 | 0.4669 | 0.55 | 0.7 | 0.51 | 0.73 | 4.04 | 0.0038 | 0.0102 | 0.0119 |
| 3625565 | 0.59 | 0.0819 | 1.31 | 0.63 | 0.611 | 0.38 | 0.77 | 0.49 | 0.68 | 3.93 | 0.0315 | 0.1383 | 0.236 |
| 3626570 | 0.66 | 0.0024 | 1.42 | 0.64 | 0.4669 | 0.57 | 0.68 | 0.51 | 0.74 | 4.45 | 0.0014 | 0.0012 | 0.0035 |
| 5626585 | 0.64 | 0.0079 | 1.26 | 0.64 | 0.5397 | 0.62 | 0.65 | 0.5 | 0.75 | 5.84 | 0.0038 | 0.0044 | 0.0072 |
| 3626730 | 0.66 | 0.0031 | 1.48 | 0.65 | 0.3949 | 0.6 | 0.68 | 0.52 | 0.75 | 5.01 | 0.0014 | 0.0015 | 0.0026 |
| 3627045 | 0.65 | 0.0063 | 1.24 | 0.61 | 0.7405 | 0.45 | 0.71 | 0.47 | 0.69 | 5.25 | 0.0386 | 0.006 | 0.0096 |
| 3628011 | 0.65 | 0.0035 | 1.34 | 0.65 | 0.3949 | 0.64 | 0.66 | 0.52 | 0.76 | 4.38 | 0.001 | 0.0069 | 0.009 |
| 3628052 | 0.65 | 0.0049 | 1.33 | 0.66 | 0.3262 | 0.53 | 0.73 | 0.53 | 0.73 | 5.43 | 0.0016 | 0.0006 | 0.0109 |
| 3628057 | 0.67 | 0.0011 | 1.27 | 0.66 | 0.3262 | 0.47 | 0.77 | 0.54 | 0.72 | 5.14 | 0.0044 | 0.0029 | 0.0026 |
| 3628471 | 0.67 | 0.0017 | 1.57 | 0.65 | 0.3949 | 0.66 | 0.65 | 0.52 | 0.77 | 6.06 | 0.0012 | 0.0019 | 0.0018 |
| 3628924 | 0.66 | 0.0032 | 1.49 | 0.65 | 0.3949 | 0.53 | 0.72 | 0.52 | 0.73 | 4.6 | 0.0034 | 0.0059 | 0.0059 |
| 3630191 | 0.67 | 0.0013 | 1.44 | 0.67 | 0.206 | 0.68 | 0.67 | 0.54 | 0.79 | 6.28 | 0.0001 | 0.0005 | 0.0011 |
| 3631414 | 0.67 | 0.0012 | 1.27 | 0.7 | 0.0837 | 0.47 | 0.83 | 0.61 | 0.73 | 4.2 | 0.0011 | 0.0009 | 0.0028 |
| 3632170 | 0.64 | 0.0069 | 1.48 | 0.6 | 0.7956 | 0.72 | 0.54 | 0.47 | 0.77 | 5.68 | 0.004 | 0.0049 | 0.0059 |
| 3633110 | 0.64 | 0.0076 | 1.38 | 0.62 | 0.6786 | 0.6 | 0.63 | 0.48 | 0.73 | 3.97 | 0.0181 | 0.0128 | 0.0112 |
| 3633223 | 0.62 | 0.0265 | 1.27 | 0.6 | 0.8429 | 0.68 | 0.55 | 0.46 | 0.75 | 6.34 | 0.0105 | 0.0166 | 0.0227 |
| 3633226 | 0.63 | 0.018 | 1.53 | 0.63 | 0.611 | 0.55 | 0.67 | 0.49 | 0.72 | 3.92 | 0.0118 | 0.0061 | 0.0099 |
| 3634074 | 0.64 | 0.0094 | 1.51 | 0.62 | 0.6786 | 0.62 | 0.62 | 0.48 | 0.74 | 5.42 | 0.0068 | 0.0066 | 0.0094 |
| 3643958 | 0.64 | 0.0089 | 1.36 | 0.62 | 0.6786 | 0.62 | 0.62 | 0.48 | 0.74 | 5.59 | 0.0059 | 0.0115 | 0.013 |
| 3645267 | 0.63 | 0.0116 | 1.25 | 0.6 | 0.7956 | 0.74 | 0.52 | 0.47 | 0.78 | 6.59 | 0.0032 | 0.0073 | 0.0097 |
| 3645314 | 0.64 | 0.0079 | 1.36 | 0.58 | 0.914 | 0.7 | 0.51 | 0.45 | 0.75 | 5.52 | 0.0194 | 0.0077 | 0.0085 |
| 3645591 | 0.61 | 0.0477 | 1.17 | 0.57 | 0.9388 | 0.57 | 0.57 | 0.44 | 0.7 | 4.87 | 0.0798 | 0.0571 | 0.0696 |
| 3647535 | 0.63 | 0.0141 | 1.39 | 0.63 | 0.611 | 0.57 | 0.66 | 0.49 | 0.73 | 5.33 | 0.0054 | 0.0127 | 0.0195 |
| 3648373 | 0.62 | 0.0192 | 1.3 | 0.6 | 0.7956 | 0.51 | 0.66 | 0.46 | 0.7 | 4.56 | 0.0461 | 0.0213 | 0.0307 |
| 3651472 | 0.63 | 0.0125 | 1.31 | 0.63 | 0.611 | 0.55 | 0.67 | 0.49 | 0.72 | 5.07 | 0.0087 | 0.0118 | 0.0152 |
| 3653349 | 0.63 | 0.0134 | 1.29 | 0.64 | 0.4669 | 0.57 | 0.68 | 0.51 | 0.74 | 4.92 | 0.0024 | 0.0178 | 0.0289 |
| 3653673 | 0.66 | 0.0026 | 1.39 | 0.64 | 0.4669 | 0.72 | 0.6 | 0.51 | 0.79 | 6.18 | 0.0003 | 0.0019 | 0.0024 |
| 3655101 | 0.61 | 0.0373 | 1.62 | 0.64 | 0.5397 | 0.57 | 0.67 | 0.5 | 0.73 | 4.38 | 0.0052 | 0.0095 | 0.0167 |
| 3658610 | 0.68 | 0.0005 | 1.35 | 0.68 | 0.1571 | 0.6 | 0.73 | 0.56 | 0.76 | 5.67 | 0.0001 | 0.0001 | 0.0006 |
| 3659201 | 0.62 | 0.0224 | 1.38 | 0.63 | 0.611 | 0.55 | 0.67 | 0.49 | 0.72 | 5.26 | 0.0072 | 0.0173 | 0.0306 |
| 3659319 | 0.7 | 0.0002 | 1.51 | 0.68 | 0.1571 | 0.62 | 0.72 | 0.56 | 0.77 | 5.12 | 0.0002 | 0.0005 | 0.0007 |
| 3660917 | 0.66 | 0.0025 | 1.43 | 0.65 | 0.3949 | 0.68 | 0.63 | 0.52 | 0.78 | 2.94 | 0.0004 | 0.0073 | 0.0079 |
| 3660927 | 0.6 | 0.0506 | 1.23 | 0.6 | 0.7956 | 0.4 | 0.72 | 0.45 | 0.68 | 4.1 | 0.0736 | 0.0422 | 0.0706 |
| 3665635 | 0.66 | 0.0033 | 1.7 | 0.58 | 0.914 | 0.57 | 0.59 | 0.44 | 0.71 | 5.46 | 0.0453 | 0.0033 | 0.0052 |
| 3656111 | 0.68 | 0.0009 | 1.34 | 0.68 | 0.1571 | 0.55 | 0.76 | 0.57 | 0.75 | 4.13 | 0.0004 | 0.0007 | 0.0014 |
| 3666869 | 0.67 | 0.0013 | 1.75 | 0.68 | 0.1571 | 0.6 | 0.73 | 0.56 | 0.76 | 4.84 | 0.0002 | 0.0012 | 0.0015 |
| 3668992 | 0.61 | 0.0405 | 1.32 | 0.63 | 0.611 | 0.51 | 0.7 | 0.49 | 0.71 | 4.51 | 0.0096 | 0.0199 | 0.0326 |
| 3671113 | 0.59 | 0.0777 | 1.09 | 0.57 | 0.9577 | 0.38 | 0.67 | 0.4 | 0.65 | 4.65 | 0.5046 | 0.0513 | 0.0935 |
| 3677863 | 0.65 | 0.0042 | 1.33 | 0.65 | 0.3949 | 0.57 | 0.7 | 0.52 | 0.74 | 5.13 | 0.0038 | 0.0079 | 0.0075 |
| 3679601 | 0.62 | 0.02 | 1.24 | 0.59 | 0.8822 | 0.49 | 0.65 | 0.44 | 0.69 | 4.34 | 0.0902 | 0.0218 | 0.034 |
| 3683054 | 0.67 | 0.0019 | 1.65 | 0.64 | 0.5397 | 0.7 | 0.6 | 0.5 | 0.78 | 5.14 | 0.0011 | 0.0009 | 0.0015 |
| 3685080 | 0.6 | 0.05 | 1.31 | 0.62 | 0.6786 | 0.53 | 0.67 | 0.48 | 0.71 | 3.73 | 0.0116 | 0.0222 | 0.0435 |
| 3687262 | 0.66 | 0.0028 | 1.31 | 0.63 | 0.611 | 0.74 | 0.56 | 0.49 | 0.79 | 8.06 | 0.0009 | 0.0017 | 0.0019 |
| 3687792 | 0.62 | 0.0289 | 1.59 | 0.64 | 0.4669 | 0.66 | 0.63 | 0.51 | 0.76 | 5.54 | 0.0014 | 0.0098 | 0.0137 |
| 3690089 | 0.65 | 0.0037 | 1.4 | 0.62 | 0.6786 | 0.7 | 0.57 | 0.49 | 0.77 | 4.87 | 0.0027 | 0.0026 | 0.0037 |
| 3692857 | 0.64 | 0.0074 | 1.44 | 0.63 | 0.611 | 0.57 | 0.66 | 0.49 | 0.73 | 5.27 | 0.0051 | 0.0088 | 0.011 |
| 3693546 | 0.67 | 0.0016 | 1.42 | 0.62 | 0.6786 | 0.64 | 0.61 | 0.48 | 0.75 | 5.36 | 0.0037 | 0.0012 | 0.0023 |
| 3693711 | 0.66 | 0.0032 | 1.59 | 0.65 | 0.3949 | 0.72 | 0.61 | 0.52 | 0.79 | 4.56 | 0.0001 | 0.002 | 0.0038 |
| 3693737 | 0.65 | 0.0049 | 1.32 | 0.67 | 0.206 | 0.66 | 0.68 | 0.54 | 0.78 | 7.29 | 0.0002 | 0.0034 | 0.004 |
| 3693746 | 0.63 | 0.0116 | 1.38 | 0.64 | 0.4669 | 0.51 | 0.72 | 0.51 | 0.72 | 4.52 | 0.007 | 0.0136 | 0.0283 |
| 3696670 | 0.65 | 0.0049 | 1.36 | 0.67 | 0.2627 | 0.57 | 0.72 | 0.54 | 0.75 | 5.19 | 0.0007 | 0.0108 | 0.0113 |
| 3697925 | 0.67 | 0.0012 | 1.28 | 0.65 | 0.3949 | 0.55 | 0.71 | 0.52 | 0.73 | 4.78 | 0.0059 | 0.0016 | 0.0025 |
| 3699559 | 0.66 | 0.0021 | 1.28 | 0.62 | 0.6786 | 0.53 | 0.67 | 0.48 | 0.71 | 4.94 | 0.0137 | 0.0019 | 0.0032 |
| 3701588 | 0.65 | 0.0042 | 1.25 | 0.6 | 0.7956 | 0.57 | 0.62 | 0.47 | 0.72 | 5.79 | 0.0464 | 0.0105 | 0.0084 |
| 3705531 | 0.63 | 0.0155 | 1.5 | 0.65 | 0.3949 | 0.45 | 0.77 | 0.53 | 0.71 | 4.03 | 0.0055 | 0.007 | 0.0163 |
| 3707211 | 0.64 | 0.0089 | 1.5 | 0.64 | 0.4669 | 0.6 | 0.67 | 0.51 | 0.74 | 5.44 | 0.0026 | 0.0078 | 0.0077 |
| 3708006 | 0.64 | 0.0104 | 1.58 | 0.63 | 0.611 | 0.7 | 0.59 | 0.49 | 0.77 | 6.36 | 0.0013 | 0.0032 | 0.005 |
| 3709275 | 0.6 | 0.0517 | 1.31 | 0.6 | 0.8429 | 0.51 | 0.65 | 0.45 | 0.7 | 5.13 | 0.0444 | 0.0247 | 0.052 |
| 3710735 | 0.65 | 0.004 | 1.28 | 0.65 | 0.3949 | 0.7 | 0.67 | 0.52 | 0.78 | 3.96 | 0.0007 | 0.0101 | 0.0101 |
| 3714102 | 0.67 | 0.0018 | 1.4 | 0.62 | 0.6786 | 0.6 | 0.63 | 0.48 | 0.73 | 5.18 | 0.0103 | 0.0017 | 0.0022 |
| 3715142 | 0.67 | 0.0018 | 1.42 | 0.67 | 0.2627 | 0.68 | 0.66 | 0.53 | 0.78 | 5.36 | 0.0002 | 0.0009 | 0.0019 |
| 3715434 | 0.6 | 0.0586 | 1.23 | 0.6 | 0.8429 | 0.47 | 0.67 | 0.45 | 0.69 | 4.94 | 0.0832 | 0.0439 | 0.0499 |
| 3716021 | 0.65 | 0.0036 | 1.39 | 0.61 | 0.7405 | 0.68 | 0.57 | 0.48 | 0.76 | 4.6 | 0.0032 | 0.0022 | 0.0051 |
| 3716674 | 0.61 | 0.036 | 1.29 | 0.6 | 0.8429 | 0.62 | 0.59 | 0.46 | 0.73 | 5.02 | 0.0279 | 0.0424 | 0.043 |
| 3716757 | 0.66 | 0.0033 | 1.26 | 0.68 | 0.1571 | 0.57 | 0.74 | 0.56 | 0.75 | 5.68 | 0.0002 | 0.0027 | 0.0044 |
| 3717647 | 0.65 | 0.0038 | 1.3 | 0.68 | 0.1571 | 0.6 | 0.73 | 0.56 | 0.76 | 5.16 | 0.0001 | 0.0097 | 0.0109 |
| 3717681 | 0.61 | 0.0401 | 1.39 | 0.66 | 0.3262 | 0.57 | 0.71 | 0.53 | 0.74 | 5.44 | 0.0021 | 0.0178 | 0.0223 |
| 3717684 | 0.63 | 0.0157 | 1.36 | 0.67 | 0.206 | 0.64 | 0.7 | 0.55 | 0.77 | 7.02 | 0.0001 | 0.0087 | 0.0103 |
| 3718837 | 0.66 | 0.0025 | 1.31 | 0.57 | 0.9388 | 0.83 | 0.43 | 0.45 | 0.81 | 6.39 | 0.0049 | 0.0032 | 0.004 |
| 3719123 | 0.62 | 0.0192 | 1.63 | 0.63 | 0.611 | 0.49 | 0.71 | 0.49 | 0.71 | 4.59 | 0.0096 | 0.0043 | 0.01 |
| 3720778 | 0.64 | 0.0083 | 1..43 | 0.67 | 0.2627 | 0.6 | 0.71 | 0.54 | 0.75 | 5.65 | 0.0004 | 0.0037 | 0.0059 |
| 3720990 | 0.66 | 0.0031 | 1.28 | 0.64 | 0.5397 | 0.62 | 0.65 | 0.5 | 0.75 | 6.82 | 0.0019 | 0.0016 | 0.0042 |
| 3722990 | 0.65 | 0.0053 | 1.3 | 0.64 | 0.4669 | 0.55 | 0.7 | 0.51 | 0.73 | 3.96 | 0.0066 | 0.004 | 0.0049 |
| 3723305 | 0.66 | 0.0027 | 1.54 | 0.6 | 0.8429 | 0.64 | 0.57 | 0.46 | 0.73 | 5.32 | 0.0222 | 0.0034 | 0.0037 |
| 3726811 | 0.63 | 0.011 | 1.39 | 0.6 | 0.7956 | 0.47 | 0.68 | 0.46 | 0.69 | 4.59 | 0.095 | 0.0156 | 0.0193 |
| 3728316 | 0.66 | 0.0026 | 1.28 | 0.64 | 0.4669 | 0.6 | 0.67 | 0.51 | 0.74 | 5.72 | 0.004 | 0.0148 | 0.004 |
| 3729194 | 0.63 | 0.012 | 1.37 | 0.64 | 0.4669 | 0.6 | 0.67 | 0.51 | 0.74 | 4.31 | 0.0047 | 0.0118 | 0.015 |
| 3729227 | 0.65 | 0.0035 | 1.38 | 0.64 | 0.4669 | 0.66 | 0.63 | 0.51 | 0.76 | 6.03 | 0.0013 | 0.0027 | 0.0037 |
| 3732475 | 0.67 | 0.0019 | 1.42 | 0.69 | 0.1164 | 0.66 | 0.71 | 0.56 | 0.78 | 5.02 | 0 | 0.0006 | 0.0012 |
| 3732477 | 0.61 | 0.0382 | 1.27 | 0.65 | 0.3949 | 0.55 | 0.71 | 0.52 | 0.73 | 4.32 | 0.0012 | 0.0183 | 0.0408 |
| 3732479 | 0.64 | 0.0078 | 1.2 | 0.62 | 0.6786 | 0.57 | 0.65 | 0.48 | 0.73 | 6 | 0.0147 | 0.0057 | 0.0084 |
| 3732480 | 0.71 | 0.0001 | 1.49 | 0.67 | 0.2627 | 0.51 | 0.76 | 0.55 | 0.73 | 3.67 | 0.0008 | 0.0001 | 0.0003 |
| 3734754 | 0.65 | 0.0044 | 1.63 | 0.6 | 0.7956 | 0.68 | 0.56 | 0.47 | 0.75 | 4.84 | 0.0055 | 0.0031 | 0.0043 |
| 3734863 | 0.62 | 0.0227 | 1.4 | 0.58 | 0.914 | 0.57 | 0.59 | 0.44 | 0.71 | 5.91 | 0.0653 | 0.0115 | 0.0184 |
| 3737304 | 0.66 | 0.0026 | 1.32 | 0.66 | 0.3262 | 0.55 | 0.72 | 0.53 | 0.74 | 5.76 | 0.0017 | 0.0058 | 0.0062 |
| 3737985 | 0.64 | 0.0107 | 1.29 | 0.64 | 0.5397 | 0.57 | 0.67 | 0.5 | 0.73 | 7.3 | 0.006 | 0.0076 | 0.0105 |
| 3738300 | 0.65 | 0.0052 | 1.35 | 0.62 | 0.6786 | 0.72 | 0.56 | 0.49 | 0.78 | 7 | 0.0018 | 0.0041 | 0.0043 |
| 3739125 | 0.64 | 0.0075 | 1.68 | 0.64 | 0.5397 | 0.55 | 0.68 | 0.5 | 0.73 | 4.35 | 0.0074 | 0.009 | 0.0091 |
| 3739523 | 0.64 | 0.0064 | 1.39 | 0.63 | 0.611 | 0.49 | 0.71 | 0.49 | 0.71 | 5.32 | 0.0265 | 0.0046 | 0.0054 |
| 3740129 | 0.68 | 0.0009 | 1.51 | 0.65 | 0.3949 | 0.68 | 0.63 | 0.52 | 0.78 | 5.48 | 0.0005 | 0.0008 | 0.0011 |
| 3740130 | 0.66 | 0.0033 | 1.74 | 0.64 | 0.5397 | 0.6 | 0.66 | 0.5 | 0.74 | 5.11 | 0.0032 | 0.0031 | 0.004 |
| 3744423 | 0.62 | 0.0205 | 1.34 | 0.62 | 0.6786 | 0.68 | 0.59 | 0.48 | 0.76 | 7.41 | 0.0033 | 0.0187 | 0.0252 |
| 3746953 | 0.66 | 0.0035 | 1.49 | 0.63 | 0.611 | 0.55 | 0.67 | 0.49 | 0.72 | 3.92 | 0.0055 | 0.0024 | 0.0074 |
| 3746967 | 0.62 | 0.021 | 1.3 | 0.63 | 0.611 | 0.49 | 0.71 | 0.49 | 0.71 | 5.09 | 0.0174 | 0.0226 | 0.0226 |
| 3747223 | 0.66 | 0.0025 | 1.46 | 0.71 | 0.0394 | 0.68 | 0.73 | 0.59 | 0.8 | 6.51 | 0 | 0.0018 | 0.0026 |
| 3748953 | 0.65 | 0.0046 | 1.5 | 0.63 | 0.611 | 0.66 | 0.61 | 0.49 | 0.76 | 4.76 | 0.0036 | 0.0026 | 0.0032 |
| 3750786 | 0.64 | 0.0106 | 1.39 | 0.62 | 0.6786 | 0.68 | 0.59 | 0.48 | 0.76 | 5.19 | 0.0022 | 0.0074 | 0.0096 |
| 3752711 | 0.62 | 0.0208 | 1.43 | 0.58 | 0.914 | 0.7 | 0.51 | 0.45 | 0.75 | 5.53 | 0.0183 | 0.0168 | 0.0209 |
| 3754530 | 0.67 | 0.0013 | 1.41 | 0.67 | 0.206 | 0.68 | 0.67 | 0.54 | 0.79 | 4.81 | 0.0001 | 0.0012 | 0.0022 |
| 3754741 | 0.65 | 0.0052 | 1.32 | 0.62 | 0.6786 | 0.55 | 0.66 | 0.48 | 0.72 | 4.2 | 0.0157 | 0.0205 | 0.0226 |
| 3756204 | 0.65 | 0.0036 | 1.29 | 0.62 | 0.6786 | 0.55 | 0.66 | 0.48 | 0.72 | 6.43 | 0.0107 | 0.0024 | 0.0045 |
| 3759590 | 0.64 | 0.0077 | 1.26 | 0.6 | 0.7956 | 0.51 | 0.66 | 0.46 | 0.7 | 4.12 | 0.063 | 0.0076 | 0.0139 |
| 3760197 | 0.64 | 0.0084 | 1.36 | 0.57 | 0.9577 | 0.62 | 0.54 | 0.43 | 0.71 | 4.29 | 0.103 | 0.0105 | 0.0095 |
| 3761752 | 0.64 | 0.0082 | 1.34 | 0.64 | 0.5397 | 0.57 | 0.67 | 0.5 | 0.73 | 4.64 | 0.0083 | 0.0121 | 0.009 |
| 3762575 | 0.66 | 0.0024 | 1.36 | 0.6 | 0.7956 | 0.55 | 0.63 | 0.46 | 0.71 | 5.25 | 0.0315 | 0.0031 | 0.0044 |
| 3764121 | 0.65 | 0.004 | 1.38 | 0.6 | 0.7956 | 0.7 | 0.55 | 0.47 | 0.76 | 6.01 | 0.0048 | 0.0032 | 0.0045 |
| 3764125 | 0.65 | 0.0043 | 1.26 | 0.64 | 0.4669 | 0.68 | 0.62 | 0.51 | 0.77 | 5.95 | 0.0004 | 0.0022 | 0.0042 |
| 3764127 | 0.61 | 0.0396 | 1.27 | 0.62 | 0.6786 | 0.51 | 0.68 | 0.48 | 0.71 | 4.58 | 0.0399 | 0.0823 | 0.0646 |
| 3764884 | 0.62 | 0.0219 | 1.45 | 0.61 | 0.7405 | 0.62 | 0.61 | 0.48 | 0.74 | 5.24 | 0.0056 | 0.0094 | 0.0145 |
| 3764921 | 0.67 | 0.0011 | 1.45 | 0.64 | 0.5397 | 0.6 | 0.66 | 0.5 | 0.74 | 6.37 | 0.0063 | 0.001 | 0.0014 |
| 3765738 | 0.62 | 0.0285 | 1.26 | 0.64 | 0.5397 | 0.53 | 0.7 | 0.5 | 0.72 | 4.53 | 0.0067 | 0.0121 | 0.025 |
| 3765761 | 0.63 | 0.0177 | 1.33 | 0.61 | 0.7405 | 0.64 | 0.6 | 0.48 | 0.74 | 5.54 | 0.007 | 0.01 | 0.0168 |
| 3765767 | 0.6 | 0.0523 | 1.34 | 0.63 | 0.611 | 0.53 | 0.68 | 0.49 | 0.72 | 3.88 | 0.0161 | 0.0448 | 0.0597 |
| 3766896 | 0.63 | 0.0116 | 1.42 | 0.63 | 0.611 | 0.55 | 0.67 | 0.49 | 0.72 | 4.97 | 0.0094 | 0.0132 | 0.0153 |
| 3768039 | 0.65 | 0.0053 | 1.33 | 0.65 | 0.3949 | 0.45 | 0.77 | 0.53 | 0.71 | 4.65 | 0.0056 | 0.0125 | 0.0177 |
| 3768121 | 0.61 | 0.0387 | 1.2 | 0.64 | 0.5397 | 0.55 | 0.68 | 0.5 | 0.73 | 4.85 | 0.0059 | 0.0523 | 0.0601 |
| 3758258 | 0.68 | 0.0008 | 1.31 | 0.64 | 0.5397 | 0.6 | 0.66 | 0.5 | 0.74 | 4.37 | 0.0036 | 0.0021 | 0.0025 |
| 3769780 | 0.63 | 0.0153 | 1.28 | 0.63 | 0.611 | 0.62 | 0.63 | 0.49 | 0.74 | 6.1 | 0.003 | 0.01 | 0.014 |
| 3770244 | 0.64 | 0.0077 | 1.27 | 0.64 | 0.4669 | 0.6 | 0.67 | 0.51 | 0.74 | 7.96 | 0.0021 | 0.0042 | 0.0065 |
| 3770564 | 0.62 | 0.0248 | 1.39 | 0.59 | 0.8822 | 0.68 | 0.54 | 0.46 | 0.75 | 5.86 | 0.0126 | 0.0163 | 0.0233 |
| 3770565 | 0.61 | 0.0445 | 1.31 | 0.59 | 0.8822 | 0.64 | 0.56 | 0.45 | 0.73 | 6.17 | 0.0204 | 0.0246 | 0.0375 |
| 3770746 | 0.64 | 0.0079 | 1.38 | 0.63 | 0.611 | 0.62 | 0.63 | 0.49 | 0.74 | 6.91 | 0.0041 | 0.005 | 0.0051 |
| 3771059 | 0.58 | 0.1262 | 1.17 | 0.61 | 0.7405 | 0.45 | 0.71 | 0.47 | 0.69 | 4.99 | 0.0524 | 0.1338 | 0.1718 |
| 3771094 | 0.65 | 0.0061 | 1.54 | 0.63 | 0.611 | 0.51 | 0.7 | 0.49 | 0.71 | 4.22 | 0.0283 | 0.006 | 0.0058 |
| 3771337 | 0.63 | 0.0118 | 1.62 | 0.62 | 0.6786 | 0.57 | 0.65 | 0.48 | 0.73 | 4.57 | 0.014 | 0.0105 | 0.0118 |
| 3776189 | 0.67 | 0.0017 | 1.42 | 0.64 | 0.4669 | 0.7 | 0.61 | 0.51 | 0.78 | 5.63 | 0.0005 | 0.002 | 0.0023 |
| 3776219 | 0.61 | 0.0303 | 1.34 | 0.61 | 0.7405 | 0.51 | 0.67 | 0.47 | 0.71 | 4.14 | 0.0576 | 0.0301 | 0.029 |
| 3776236 | 0.62 | 0.0216 | 1.3 | 0.64 | 0.5397 | 0.38 | 0.78 | 0.5 | 0.69 | 3.83 | 0.0895 | 0.0201 | 0.0168 |
| 3776446 | 0.65 | 0.0063 | 1.83 | 0.61 | 0.7405 | 0.6 | 0.62 | 0.47 | 0.73 | 4.65 | 0.011 | 0.0043 | 0.0067 |
| 3776461 | 0.66 | 0.0035 | 1.38 | 0.61 | 0.7405 | 0.57 | 0.63 | 0.47 | 0.72 | 6.35 | 0.0166 | 0.0032 | 0.0038 |
| 3778266 | 0.69 | 0.0004 | 1.3 | 0.66 | 0.3262 | 0.62 | 0.68 | 0.53 | 0.76 | 6.28 | 0.0007 | 0.0007 | 0.0008 |
| 3778629 | 0.68 | 0.0006 | 1.5 | 0.67 | 0.2627 | 0.68 | 0.66 | 0.53 | 0.78 | 6.12 | 0.0002 | 0.0005 | 0.001 |
| 3778630 | 0.67 | 0.0016 | 1.42 | 0.59 | 0.8822 | 0.64 | 0.56 | 0.45 | 0.73 | 6.07 | 0.0233 | 0.0015 | 0.0022 |
| 3779850 | 0.65 | 0.0059 | 1.27 | 0.61 | 0.7405 | 0.3 | 0.79 | 0.45 | 0.66 | 4.46 | 0.2066 | 0.0216 | 0.0261 |
| 3781686 | 0.58 | 0.1481 | 1.2 | 0.5 | 0.9388 | 0.26 | 0.76 | 0.38 | 0.64 | 3.81 | 0.7859 | 0.2517 | 0.3776 |
| 3781689 | 0.65 | 0.0035 | 1.33 | 0.62 | 0.6786 | 0.55 | 0.66 | 0.48 | 0.72 | 5.5 | 0.0144 | 0.0028 | 0.0054 |
| 3783364 | 0.65 | 0.004 | 1.29 | 0.59 | 0.8822 | 0.57 | 0.6 | 0.45 | 0.71 | 6.36 | 0.0381 | 0.0027 | 0.0053 |
| 3787934 | 0.67 | 0.0013 | 1.31 | 0.67 | 0.2627 | 0.6 | 0.71 | 0.54 | 0.75 | 6.23 | 0.0006 | 0.0003 | 0.0008 |
| 3789817 | 0.65 | 0.0035 | 1.47 | 0.64 | 0.4669 | 0.6 | 0.67 | 0.51 | 0.74 | 5.01 | 0.002 | 0.0033 | 0.0053 |
| 3795442 | 0.63 | 0.0153 | 1.26 | 0.6 | 0.8429 | 0.43 | 0.7 | 0.44 | 0.68 | 4.16 | 0.1521 | 0.0254 | 0.0278 |
| 3796554 | 0.6 | 0.0529 | 1.21 | 0.57 | 0.9577 | 0.53 | 0.59 | 0.42 | 0.69 | 3.93 | 0.1323 | 0.0387 | 0.0638 |
| 3798470 | 0.67 | 0.0014 | 1.43 | 0.65 | 0.3949 | 0.55 | 0.71 | 0.52 | 0.73 | 6.12 | 0.0023 | 0.001 | 0.002 |
| 3800623 | 0.6 | 0.0619 | 1.27 | 0.59 | 0.8822 | 0.55 | 0.61 | 0.45 | 0.7 | 4.69 | 0.0615 | 0.0437 | 0.0506 |
| 3804170 | 0.6 | 0.004 | 1.33 | 0.61 | 0.7405 | 0.62 | 0.61 | 0.48 | 0.74 | 4.18 | 0.013 | 0.0044 | 0.0062 |
| 3804202 | 0.61 | 0.0356 | 1.29 | 0.62 | 0.6786 | 0.53 | 0.67 | 0.48 | 0.71 | 4.53 | 0.0156 | 0.0195 | 0.0308 |
| 3806255 | 0.66 | 0.0029 | 1.4 | 0.64 | 0.5397 | 0.68 | 0.61 | 0.5 | 0.77 | 6.51 | 0.0011 | 0.0018 | 0.0026 |
| 3806256 | 0.62 | 0.0197 | 1.3 | 0.62 | 0.6786 | 0.57 | 0.65 | 0.48 | 0.73 | 4.27 | 0.0173 | 0.0134 | 0.0211 |
| 3807476 | 0.64 | 0.0101 | 1.46 | 0.65 | 0.3949 | 0.57 | 0.7 | 0.52 | 0.74 | 4.79 | 0.0012 | 0.0045 | 0.0093 |
| 3807570 | 0.61 | 0.0369 | 1.14 | 0.63 | 0.611 | 0.36 | 0.78 | 0.49 | 0.68 | 4.41 | 0.0377 | 0.0102 | 0.0292 |
| 3807629 | 0.62 | 0.0189 | 1.2 | 0.64 | 0.5397 | 0.43 | 0.76 | 0.5 | 0.7 | 4.45 | 0.0242 | 0.004 | 0.0124 |
| 3808602 | 0.67 | 0.001 | 1.41 | 0.64 | 0.4669 | 0.7 | 0.61 | 0.51 | 0.78 | 7.12 | 0.0005 | 0.0009 | 0.0011 |
| 3809343 | 0.65 | 0.0062 | 1.42 | 0.59 | 0.8822 | 0.66 | 0.55 | 0.46 | 0.74 | 5.22 | 0.0186 | 0.0045 | 0.0064 |
| 3809673 | 0.64 | 0.0076 | 1.36 | 0.62 | 0.6786 | 0.68 | 0.59 | 0.48 | 0.76 | 6.13 | 0.0028 | 0.0056 | 0.0077 |
| 3809834 | 0.67 | 0.0012 | 1.27 | 0.66 | 0.3262 | 0.51 | 0.74 | 0.53 | 0.73 | 5.06 | 0.0065 | 0.0012 | 0.0017 |
| 3813298 | 0.66 | 0.0027 | 1.35 | 0.64 | 0.5397 | 0.51 | 0.71 | 0.5 | 0.72 | 5.9 | 0.01 | 0.0007 | 0.0025 |
| 3813299 | 0.6 | 0.05 | 1.17 | 0.6 | 0.7956 | 0.45 | 0.7 | 0.46 | 0.69 | 4.53 | 0.058 | 0.0182 | 0.0544 |
| 3815669 | 0.62 | 0.0224 | 1.42 | 0.58 | 0.914 | 0.64 | 0.55 | 0.45 | 0.73 | 4.46 | 0.0381 | 0.0168 | 0.0211 |
| 3816402 | 0.63 | 0.012 | 1.41 | 0.62 | 0.6786 | 0.6 | 0.63 | 0.48 | 0.73 | 4.6 | 0.0104 | 0.0143 | 0.0182 |
| 3820658 | 0.63 | 0.0147 | 1.29 | 0.57 | 0.9577 | 0.74 | 0.46 | 0.44 | 0.76 | 5.83 | 0.0247 | 0.0158 | 0.0184 |
| 3820705 | 0.63 | 0.0177 | 1.23 | 0.6 | 0.8429 | 0.62 | 0.59 | 0.46 | 0.73 | 6.02 | 0.0203 | 0.0154 | 0.0197 |
| 3820721 | 0.63 | 0.0132 | 1.3 | 0.58 | 0.914 | 0.7 | 0.51 | 0.45 | 0.75 | 5.6 | 0.0253 | 0.0141 | 0.0153 |
| 3820752 | 0.62 | 0.0285 | 1.22 | 0.6 | 0.8429 | 0.55 | 0.62 | 0.46 | 0.71 | 4.6 | 0.0444 | 0.038 | 0.0291 |
| 3822055 | 0.62 | 0.0296 | 1.37 | 0.61 | 0.7405 | 0.6 | 0.62 | 0.47 | 0.73 | 5.94 | 0.0089 | 0.0203 | 0.0324 |
| 3825428 | 0.65 | 0.0046 | 1.55 | 0.61 | 0.7405 | 0.64 | 0.6 | 0.48 | 0.74 | 5.55 | 0.006 | 0.0035 | 0.0041 |
| 3829039 | 0.65 | 0.0043 | 1.47 | 0.63 | 0.611 | 0.53 | 0.68 | 0.49 | 0.72 | 5.01 | 0.0099 | 0.0038 | 0.006 |
| 3829810 | 0.66 | 0.0027 | 1.52 | 0.6 | 0.7956 | 0.66 | 0.57 | 0.47 | 0.75 | 5.69 | 0.0069 | 0.0026 | 0.0037 |
| 3831128 | 0.63 | 0.0147 | 1.71 | 0.64 | 0.4669 | 0.66 | 0.63 | 0.51 | 0.76 | 4.79 | 0.0007 | 0.0056 | 0.0076 |
| 3831280 | 0.64 | 0.0073 | 1.28 | 0.62 | 0.6786 | 0.62 | 0.62 | 0.48 | 0.74 | 7.53 | 0.0063 | 0.0038 | 0.0054 |
| 3835412 | 0.64 | 0.007 | 1.57 | 0.62 | 0.6786 | 0.51 | 0.68 | 0.48 | 0.71 | 3.89 | 0.026 | 0.0194 | 0.0239 |
| 3837493 | 0.63 | 0.0145 | 1.29 | 0.59 | 0.8822 | 0.53 | 0.62 | 0.45 | 0.7 | 5.53 | 0.081 | 0.015 | 0.0197 |
| 3838612 | 0.63 | 0.017 | 1.31 | 0.64 | 0.4669 | 0.57 | 0.68 | 0.51 | 0.74 | 5.57 | 0.0031 | 0.0112 | 0.0165 |
| 3838790 | 0.64 | 0.0073 | 1.23 | 0.63 | 0.611 | 0.45 | 0.73 | 0.49 | 0.7 | 4.89 | 0.0206 | 0.0103 | 0.0144 |
| 3840344 | 0.67 | 0.0014 | 1.39 | 0.61 | 0.7405 | 0.57 | 0.63 | 0.47 | 0.72 | 5.71 | 0.023 | 0.0007 | 0.0014 |
| 3841271 | 0.61 | 0.0307 | 1.24 | 0.63 | 0.611 | 0.47 | 0.72 | 0.49 | 0.7 | 4.87 | 0.0209 | 0.0269 | 0.0454 |
| 3843668 | 0.67 | 0.0013 | 1.32 | 0.66 | 0.3262 | 0.51 | 0.74 | 0.53 | 0.73 | 4.81 | 0.0019 | 0.0024 | 0.0037 |
| 3845710 | 0.66 | 0.0032 | 1.4 | 0.6 | 0.8129 | 0.74 | 0.51 | 0.47 | 0.78 | 6.23 | 0.0063 | 0.0046 | 0.0045 |
| 3846115 | 0.61 | 0.0477 | 1.36 | 0.65 | 0.3949 | 0.6 | 0.68 | 0.52 | 0.75 | 5.06 | 0.0019 | 0.035 | 0.043 |
| 3846291 | 0.61 | 0.0327 | 1.26 | 0.61 | 0.7405 | 0.57 | 0.63 | 0.47 | 0.72 | 4.48 | 0.0218 | 0.044 | 0.0396 |
| 3846561 | 0.64 | 0.008 | 1.29 | 0.6 | 0.8429 | 0.68 | 0.55 | 0.46 | 0.75 | 7.21 | 0.0082 | 0.0079 | 0.0119 |
| 3846784 | 0.63 | 0.0113 | 1.53 | 0.6 | 0.7956 | 0.7 | 0.55 | 0.47 | 0.76 | 5.8 | 0.0061 | 0.0049 | 0.0068 |
| 3847357 | 0.65 | 0.0041 | 1.41 | 0.62 | 0.6786 | 0.72 | 0.56 | 0.49 | 0.78 | 6.78 | 0.0021 | 0.0048 | 0.0053 |
| 3849774 | 0.61 | 0.0327 | 1.33 | 0.62 | 0.6786 | 0.51 | 0.68 | 0.48 | 0.71 | 6.01 | 0.0264 | 0.0369 | 0.0358 |
| 3849776 | 0.67 | 0.0019 | 1.32 | 0.66 | 0.3262 | 0.55 | 0.72 | 0.53 | 0.74 | 5.29 | 0.0016 | 0.0025 | 0.0039 |
| 3850502 | 0.67 | 0.0016 | 1.36 | 0.64 | 0.4669 | 0.7 | 0.61 | 0.51 | 0.78 | 5.58 | 0.0006 | 0.0018 | 0.0015 |
| 3851604 | 0.63 | 0.0141 | 1.29 | 0.63 | 0.611 | 0.68 | 0.6 | 0.49 | 0.77 | 6.87 | 0.0023 | 0.0116 | 0.0134 |
| 3851902 | 0.64 | 0.0089 | 1.45 | 0.64 | 0.4669 | 0.66 | 0.63 | 0.51 | 0.76 | 5.42 | 0.0008 | 0.0065 | 0.0118 |
| 3854371 | 0.65 | 0.0062 | 1.46 | 0.65 | 0.3949 | 0.47 | 0.76 | 0.52 | 0.71 | 3.76 | 0.006 | 0.0124 | 0.0185 |
| 3855101 | 0.65 | 0.0045 | 1.28 | 0.64 | 0.5397 | 0.66 | 0.62 | 0.5 | 0.76 | 6.28 | 0.0013 | 0.0035 | 0.0061 |
| 3856046 | 0.61 | 0.0364 | 1.3 | 0.61 | 0.7405 | 0.36 | 0.76 | 0.46 | 0.67 | 4.68 | 0.1657 | 0.0462 | 0.0432 |
| 3857120 | 0.6 | 0.0511 | 1.22 | 0.63 | 0.611 | 0.49 | 0.71 | 0.49 | 0.71 | 4.3 | 0.02 | 0.0261 | 0.0385 |
| 3857884 | 0.69 | 0.0003 | 1.32 | 0.69 | 0.1164 | 0.68 | 0.7 | 0.56 | 0.79 | 5.03 | 0 | 0.0003 | 0.0007 |
| 3862168 | 0.65 | 0.0048 | 1.3 | 0.62 | 0.6786 | 0.72 | 0.56 | 0.49 | 0.78 | 8.35 | 0.0018 | 0.0049 | 0.0058 |
| 3862473 | 0.64 | 0.0107 | 1.27 | 0.58 | 0.914 | 0.62 | 0.56 | 0.45 | 0.72 | 6.29 | 0.0381 | 0.0067 | 0.0061 |
| 3867099 | 0.61 | 0.0335 | 1.35 | 0.66 | 0.3262 | 0.6 | 0.7 | 0.53 | 0.75 | 5.64 | 0.0012 | 0.0235 | 0.0307 |
| 3869659 | 0.6 | 0.0676 | 1.33 | 0.6 | 0.7956 | 0.53 | 0.65 | 0.46 | 0.71 | 4.49 | 0.0612 | 0.064 | 0.0637 |
| 3869971 | 0.66 | 0.0027 | 1.45 | 0.65 | 0.3949 | 0.51 | 0.73 | 0.52 | 0.72 | 4.79 | 0.0054 | 0.0015 | 0.0027 |
| 3870593 | 0.67 | 0.0013 | 1.32 | 0.69 | 0.1164 | 0.7 | 0.68 | 0.56 | 0.8 | 6.4 | 0 | 0.0012 | 0.0015 |
| 3872197 | 0.66 | 0.0021 | 1.77 | 0.7 | 0.0837 | 0.7 | 0.7 | 0.57 | 0.8 | 5.29 | 0 | 0.0009 | 0.0011 |
| 3872208 | 0.64 | 0.0093 | 1.26 | 0.63 | 0.611 | 0.62 | 0.63 | 0.49 | 0.74 | 5.59 | 0.0056 | 0.0083 | 0.0088 |
| 3874114 | 0.62 | 0.0239 | 1.26 | 0.64 | 0.5397 | 0.53 | 0.7 | 0.5 | 0.72 | 3.09 | 0.0103 | 0.0407 | 0.0433 |
| 3874280 | 0.66 | 0.0033 | 1.36 | 0.62 | 0.6786 | 0.72 | 0.56 | 0.49 | 0.78 | 7.63 | 0.0019 | 0.0027 | 0.0032 |
| 3875278 | 0.61 | 0.0351 | 1.34 | 0.6 | 0.7956 | 0.6 | 0.61 | 0.47 | 0.72 | 4.85 | 0.0204 | 0.0204 | 0.0283 |
| 3878051 | 0.62 | 0.0216 | 1.56 | 0.64 | 0.5397 | 0.57 | 0.67 | 0.5 | 0.73 | 4.85 | 0.0043 | 0.0109 | 0.0122 |
| 3878053 | 0.68 | 0.0009 | 1.38 | 0.67 | 0.2627 | 0.7 | 0.65 | 0.53 | 0.79 | 7.19 | 0.0001 | 0.0006 | 0.0009 |
| 3879490 | 0.62 | 0.0194 | 1.31 | 0.61 | 0.7405 | 0.51 | 0.67 | 0.47 | 0.71 | 3.19 | 0.0306 | 0.0149 | 0.0286 |
| 3881731 | 0.66 | 0.0032 | 1.61 | 0.6 | 0.7956 | 0.66 | 0.57 | 0.47 | 0.75 | 4.79 | 0.0098 | 0.003 | 0.0032 |
| 3881914 | 0.62 | 0.0221 | 0.28 | 0.59 | 0.8822 | 0.6 | 0.59 | 0.45 | 0.72 | 6.02 | 0.0309 | 0.0181 | 0.0348 |
| 3882710 | 0.63 | 0.0143 | 1.48 | 0.61 | 0.7405 | 0.62 | 0.61 | 0.48 | 0.74 | 6.5 | 0.0107 | 0.01 | 0.0129 |
| 3882867 | 0.65 | 0.0042 | 1.48 | 0.65 | 0.3949 | 0.55 | 0.71 | 0.52 | 0.73 | 5.07 | 0.0034 | 0.0064 | 0.0082 |
| 3883424 | 0.69 | 0.0003 | 1.3 | 0.64 | 0.4669 | 0.66 | 0.63 | 0.51 | 0.76 | 6.99 | 0.0011 | 0.0003 | 0.0007 |
| 3883533 | 0.67 | 0.0016 | 1.31 | 0.7 | 0.0837 | 0.7 | 0.7 | 0.57 | 0.8 | 6.28 | 0 | 0.0012 | 0.0017 |
| 3883537 | 0.63 | 0.0126 | 1.35 | 0.64 | 0.4669 | 0.55 | 0.7 | 0.51 | 0.73 | 6.1 | 0.0035 | 0.0079 | 0.0117 |
| 3883987 | 0.69 | 0.0003 | 1.3 | 0.6 | 0.7956 | 0.81 | 0.49 | 0.48 | 0.82 | 5.1 | 0.001 | 0.0005 | 0.0006 |
| 3884147 | 0.66 | 0.0026 | 1.36 | 0.64 | 0.4669 | 0.66 | 0.63 | 0.51 | 0.76 | 5.72 | 0.0007 | 0.0034 | 0.0057 |
| 3884334 | 0.69 | 0.0004 | 1.33 | 0.64 | 0.5397 | 0.38 | 0.78 | 0.5 | 0.69 | 4.08 | 0.0538 | 0.0053 | 0.0029 |
| 3884673 | 0.61 | 0.0339 | 1.26 | 0.64 | 0.4669 | 0.45 | 0.76 | 0.51 | 0.7 | 4.09 | 0.0145 | 0.0108 | 0.0227 |
| 3884709 | 0.68 | 0.0006 | 1.26 | 0.68 | 0.1571 | 0.55 | 0.76 | 0.57 | 0.75 | 5.2 | 0.0002 | 0.0005 | 0.0008 |
| 3885711 | 0.62 | 0.0205 | 1.26 | 0.64 | 0.5397 | 0.72 | 0.59 | 0.5 | 0.79 | 6.2 | 0.0006 | 0.0116 | 0.0164 |
| 3886656 | 0.66 | 0.0027 | 1.42 | 0.65 | 0.3949 | 0.51 | 0.73 | 0.52 | 0.72 | 5.21 | 0.0059 | 0.005 | 0.006 |
| 3886907 | 0.67 | 0.0011 | 1.34 | 0.71 | 0.0583 | 0.62 | 0.76 | 0.59 | 0.78 | 6.13 | 0 | 0.0009 | 0.0025 |
| 3887661 | 0.64 | 0.0076 | 1.25 | 0.65 | 0.3949 | 0.47 | 0.76 | 0.52 | 0.71 | 3.9 | 0.0078 | 0.0067 | 0.011 |
| 3888081 | 0.65 | 0.0039 | 1.34 | 0.67 | 0.2627 | 0.57 | 0.72 | 0.54 | 0.75 | 5.12 | 0.0004 | 0.002 | 0.0055 |
| 3888182 | 0.65 | 0.0055 | 1.35 | 0.6 | 0.7956 | 0.74 | 0.52 | 0.47 | 0.78 | 6.68 | 0.003 | 0.006 | 0.0055 |
| 3888253 | 0.67 | 0.0016 | 1.34 | 0.66 | 0.3262 | 0.62 | 0.68 | 0.53 | 0.76 | 5.99 | 0.0005 | 0.0015 | 0.002 |
| 3888289 | 0.65 | 0.0044 | 1.52 | 0.63 | 0.611 | 0.7 | 0.59 | 0.49 | 0.77 | 5.77 | 0.002 | 0.0022 | 0.0035 |
| 3888494 | 0.63 | 0.0137 | 1.3 | 0.63 | 0.611 | 0.62 | 0.63 | 0.49 | 0.74 | 6.08 | 0.0054 | 0.0136 | 0.0152 |
| 3888936 | 0.63 | 0.0118 | 1.28 | 0.61 | 0.7405 | 0.55 | 0.65 | 0.47 | 0.72 | 5.35 | 0.0332 | 0.0155 | 0.014 |
| 3889140 | 0.68 | 0.0009 | 1.29 | 0.64 | 0.5397 | 0.62 | 0.65 | 0.5 | 0.75 | 6.04 | 0.0016 | 0.0001 | 0.0011 |
| 3889782 | 0.66 | 0.0031 | 1.5 | 0.63 | 0.611 | 0.53 | 0.68 | 0.49 | 0.72 | 3.77 | 0.0084 | 0.0023 | 0.0043 |
| 3891221 | 0.62 | 0.0197 | 1.39 | 0.62 | 0.6786 | 0.6 | 0.63 | 0.48 | 0.73 | 4.77 | 0.0144 | 0.0114 | 0.0191 |
| 3891257 | 0.69 | 0.0003 | 1.34 | 0.67 | 0.2627 | 0.7 | 0.65 | 0.53 | 0.79 | 7.71 | 0.0001 | 0.0002 | 0.0005 |
| 3892672 | 0.67 | 0.0015 | 1.27 | 0.66 | 0.3262 | 0.66 | 0.66 | 0.53 | 0.77 | 7.8 | 0.0003 | 0.0013 | 0.0033 |
| 3894074 | 0.62 | 0.0258 | 1.31 | 0.61 | 0.7405 | 0.53 | 0.66 | 0.41 | 0.71 | 3.6 | 0.0323 | 0.0068 | 0.0165 |
| 3894508 | 0.67 | 0.0011 | 1.54 | 0.66 | 0.3262 | 0.64 | 0.67 | 0.53 | 0.76 | 5.69 | 0.0005 | 0.0006 | 0.0008 |
| 3894602 | 0.63 | 0.011 | 1.47 | 0.62 | 0.6786 | 0.64 | 0.61 | 0.48 | 0.75 | 5.61 | 0.0054 | 0.0069 | 0.0081 |
| 3895000 | 0.64 | 0.0098 | 1.4 | 0.66 | 0.3262 | 0.66 | 0.66 | 0.53 | 0.77 | 6.34 | 0.0004 | 0.007 | 0.0085 |
| 3896202 | 0.61 | 0.0311 | 1.33 | 0.59 | 0.8822 | 0.51 | 0.63 | 0.44 | 0.69 | 5.28 | 0.0961 | 0.026 | 0.033 |
| 3898306 | 0.59 | 0.0845 | 1.27 | 0.6 | 0.8429 | 0.51 | 0.65 | 0.45 | 0.7 | 3.14 | 0.1419 | 0.3259 | 0.2322 |
| 3901666 | 0.63 | 0.0155 | 1.39 | 0.6 | 0.8429 | 0.68 | 0.55 | 0.46 | 0.75 | 6.28 | 0.0099 | 0.0147 | 0.0219 |
| 3902984 | 0.66 | 0.0031 | 1.41 | 0.61 | 0.7405 | 0.55 | 0.65 | 0.47 | 0.72 | 5.3 | 0.023 | 0.0045 | 0.0054 |
| 3903290 | 0.67 | 0.0016 | 1.36 | 0.66 | 0.3262 | 0.66 | 0.66 | 0.53 | 0.77 | 6.63 | 0.0004 | 0.0012 | 0.0012 |
| 3904028 | 0.63 | 0.0121 | 1.35 | 0.6 | 0.7956 | 0.26 | 0.8 | 0.43 | 0.65 | 4.19 | 0.4545 | 0.0146 | 0.0257 |
| 3904029 | 0.67 | 0.0017 | 1.31 | 0.68 | 0.1571 | 0.64 | 0.71 | 0.56 | 0.77 | 8.48 | 0.0002 | 0.0004 | 0.0012 |
| 3904255 | 0.66 | 0.0026 | 1.28 | 0.61 | 0.7405 | 0.66 | 0.59 | 0.48 | 0.75 | 5.33 | 0.0098 | 0.0063 | 0.0058 |
| 3904257 | 0.66 | 0.0025 | 1.33 | 0.64 | 0.4669 | 0.77 | 0.57 | 0.51 | 0.81 | 6.08 | 0.0002 | 0.0021 | 0.002 |
| 3904276 | 0.64 | 0.0074 | 1.25 | 0.61 | 0.7405 | 0.53 | 0.66 | 0.47 | 0.71 | 4.43 | 0.04 | 0.0169 | 0.0186 |
| 3904281 | 0.61 | 0.0311 | 1.23 | 0.64 | 0.4669 | 0.45 | 0.76 | 0.51 | 0.7 | 5.01 | 0.0066 | 0.0115 | 0.018 |
| 3906196 | 0.65 | 0.005 | 1.35 | 0.64 | 0.4669 | 0.64 | 0.65 | 0.51 | 0.76 | 4.26 | 0.0013 | 0.0033 | 0.0065 |
| 3907788 | 0.62 | 0.0197 | 1.49 | 0.63 | 0.611 | 0.68 | 0.6 | 0.49 | 0.77 | 4.76 | 0.0018 | 0.0149 | 0.0156 |
| 3908789 | 0.65 | 0.0037 | 1.33 | 0.67 | 0.2627 | 0.68 | 0.66 | 0.53 | 0.78 | 7.31 | 0.0002 | 0.0015 | 0.0018 |
| 3909376 | 0.61 | 0.0351 | 1.28 | 0.61 | 0.7405 | 0.49 | 0.68 | 0.47 | 0.7 | 3.78 | 0.0202 | 0.0082 | 0.0186 |
| 3911474 | 0.67 | 0.001 | 1.47 | 0.64 | 0.5397 | 0.68 | 0.61 | 0.5 | 0.77 | 6.61 | 0.0012 | 0.0006 | 0.0009 |
| 3911564 | 0.69 | 0.0004 | 1.46 | 0.72 | 0.0257 | 0.7 | 0.73 | 0.6 | 0.81 | 5.98 | 0 | 0.0001 | 0.0003 |
| 3911706 | 0.66 | 0.0028 | 1.33 | 0.67 | 0.206 | 0.64 | 0.7 | 0.55 | 0.77 | 5.67 | 0.0003 | 0.002 | 0.0035 |
| 3911738 | 0.69 | 0.0004 | 1.34 | 0.64 | 0.5397 | 0.74 | 0.57 | 0.5 | 0.8 | 7.01 | 0.0008 | 0.0005 | 0.0007 |
| 3911801 | 0.66 | 0.0032 | 1.5 | 0.64 | 0.4669 | 0.66 | 0.63 | 0.51 | 0.76 | 5.97 | 0.0009 | 0.0013 | 0.0025 |
| 3911815 | 0.63 | 0.0147 | 1.61 | 0.64 | 0.4669 | 0.64 | 0.65 | 0.51 | 0.76 | 5.17 | 0.0017 | 0.0056 | 0.0084 |
| 3913564 | 0.62 | 0.021 | 1.33 | 0.58 | 0.914 | 0.6 | 0.57 | 0.44 | 0.7 | 4.54 | 0.0681 | 0.0283 | 0.0207 |
| 3915125 | 0.68 | 0.0007 | 1.5 | 0.65 | 0.3949 | 0.45 | 0.77 | 0.53 | 0.71 | 3.94 | 0.0104 | 0.0006 | 0.0011 |
| 3917896 | 0.67 | 0.0013 | 1.51 | 0.64 | 0.4669 | 0.66 | 0.63 | 0.51 | 0.76 | 5.34 | 0.001 | 0.0018 | 0.0021 |
| 3918586 | 0.66 | 0.0029 | 1.54 | 0.69 | 0.1164 | 0.53 | 0.78 | 0.58 | 0.74 | 3.89 | 0.0002 | 0.0015 | 0.0026 |
| 3918651 | 0.6 | 0.0729 | 1.27 | 0.6 | 0.8429 | 0.55 | 0.62 | 0.46 | 0.71 | 4.26 | 0.0417 | 0.0294 | 0.047 |
| 3918723 | 0.67 | 0.0016 | 1.36 | 0.66 | 0.3262 | 0.7 | 0.63 | 0.52 | 0.79 | 5.83 | 0.0002 | 0.0011 | 0.0024 |
| 3918734 | 0.65 | 0.0045 | 1.42 | 0.64 | 0.4669 | 0.6 | 0.67 | 0.51 | 0.74 | 4.32 | 0.0012 | 0.0022 | 0.0053 |
| 3919024 | 0.66 | 0.0033 | 1.4 | 0.65 | 0.3949 | 0.81 | 0.56 | 0.51 | 0.84 | 6.04 | 0.0001 | 0.0017 | 0.0017 |
| 3920401 | 0.65 | 0.0051 | 1.49 | 0.65 | 0.3949 | 0.55 | 0.71 | 0.52 | 0.73 | 4.13 | 0.0024 | 0.0094 | 0.0119 |
| 3920456 | 0.65 | 0.0036 | 1.28 | 0.62 | 0.6786 | 0.7 | 0.57 | 0.49 | 0.77 | 6.29 | 0.0018 | 0.002 | 0.0031 |
| 3920487 | 0.65 | 0.004 | 1.33 | 0.64 | 0.4669 | 0.62 | 0.66 | 0.51 | 0.75 | 4.86 | 0.0025 | 0.0027 | 0.0048 |
| 3922023 | 0.63 | 0.0151 | 1.41 | 0.64 | 0.5397 | 0.34 | 0.8 | 0.5 | 0.68 | 4.47 | 0.0392 | 0.0078 | 0.0196 |
| 3922993 | 0.66 | 0.0023 | 1.4 | 0.66 | 0.3262 | 0.7 | 0.63 | 0.52 | 0.79 | 4.05 | 0.0002 | 0.0047 | 0.0096 |
| 3924758 | 0.66 | 0.0021 | 1.41 | 0.63 | 0.611 | 0.51 | 0.7 | 0.49 | 0.71 | 4.23 | 0.0233 | 0.0111 | 0.0087 |
| 3925641 | 0.66 | 0.0024 | 1.32 | 0.67 | 0.2627 | 0.45 | 0.79 | 0.55 | 0.71 | 5.47 | 0.0014 | 0.0037 | 0.0061 |
| 3926090 | 0.62 | 0.023 | 1.3 | 0.66 | 0.3262 | 0.47 | 0.77 | 0.54 | 0.72 | 4.34 | 0.003 | 0.0047 | 0.0076 |
| 3927180 | 0.62 | 0.0202 | 1.49 | 0.61 | 0.7405 | 0.55 | 0.65 | 0.47 | 0.72 | 4.11 | 0.0286 | 0.0124 | 0.0136 |
| 3927228 | 0.64 | 0.0068 | 1.32 | 0.58 | 0.914 | 0.79 | 0.46 | 0.46 | 0.79 | 6.3 | 0.0064 | 0.0086 | 0.0088 |
| 3927229 | 0.65 | 0.0049 | 1.42 | 0.63 | 0.611 | 0.74 | 0.56 | 0.49 | 0.79 | 5.47 | 0.001 | 0.0042 | 0.0039 |
| 3928670 | 0.65 | 0.0039 | 1.4 | 0.64 | 0.5397 | 0.66 | 0.62 | 0.5 | 0.76 | 5.02 | 0.0021 | 0.0024 | 0.0044 |
| 3929943 | 0.63 | 0.0128 | 1.38 | 0.6 | 0.7956 | 0.64 | 0.59 | 0.47 | 0.74 | 6.55 | 0.0101 | 0.004 | 0.0092 |
| 3934699 | 0.6 | 0.0511 | 1.4 | 0.62 | 0.6786 | 0.62 | 0.62 | 0.48 | 0.74 | 4.85 | 0.0096 | 0.0353 | 0.0411 |
| 3935246 | 0.6 | 0.0633 | 1.36 | 0.64 | 0.5397 | 0.64 | 0.63 | 0.5 | 0.7 | 4.96 | 0.0017 | 0.0527 | 0.0596 |
| 3935301 | 0.64 | 0.0103 | 1.34 | 0.64 | 0.4669 | 0.66 | 0.63 | 0.51 | 0.76 | 7.04 | 0.001 | 0.0068 | 0.0082 |
| 3936897 | 0.66 | 0.0022 | 1.63 | 0.65 | 0.3949 | 0.55 | 0.71 | 0.52 | 0.73 | 4.74 | 0.0033 | 0.0032 | 0.0033 |
| 3939407 | 0.66 | 0.0029 | 1.35 | 0.64 | 0.5397 | 0.72 | 0.59 | 0.5 | 0.79 | 6.88 | 0.0009 | 0.0024 | 0.0025 |
| 3942668 | 0.67 | 0.0015 | 1.35 | 0.64 | 0.4669 | 0.66 | 0.63 | 0.51 | 0.76 | 7.03 | 0.0015 | 0.0007 | 0.0011 |
| 3945331 | 0.65 | 0.0037 | 1.5 | 0.61 | 0.7405 | 0.62 | 0.61 | 0.48 | 0.74 | 5.07 | 0.0167 | 0.0063 | 0.0057 |
| 3945370 | 0.61 | 0.0477 | 1.38 | 0.65 | 0.3949 | 0.53 | 0.72 | 0.52 | 0.73 | 5.14 | 0.0021 | 0.0318 | 0.0333 |
| 3946374 | 0.66 | 0.0024 | 1.34 | 0.64 | 0.4669 | 0.45 | 0.76 | 0.51 | 0.7 | 4.9 | 0.0121 | 0.0033 | 0.005 |
| 3946589 | 0.61 | 0.0477 | 1.34 | 0.61 | 0.7405 | 0.57 | 0.63 | 0.47 | 0.72 | 5.01 | 0.0211 | 0.0465 | 0.0531 |
| 3946677 | 0.6 | 0.0517 | 1.3 | 0.61 | 0.7405 | 0.47 | 0.7 | 0.47 | 0.7 | 4.18 | 0.06 | 0.0388 | 0.0527 |
| 3953804 | 0.72 | 0 | 1.35 | 0.69 | 0.1164 | 0.7 | 0.68 | 0.56 | 0.8 | 4.78 | 0 | 0.0001 | 0.0003 |
| 3956591 | 0.63 | 0.0128 | 1.34 | 0.6 | 0.7956 | 0.66 | 0.57 | 0.47 | 0.75 | 7.26 | 0.0177 | 0.0148 | 0.0097 |
| 3957262 | 0.62 | 0.0197 | 1.44 | 0.64 | 0.5397 | 0.51 | 0.71 | 0.5 | 0.72 | 4.97 | 0.0151 | 0.0191 | 0.0191 |
| 3959259 | 0.63 | 0.0153 | 1.24 | 0.6 | 0.7956 | 0.6 | 0.61 | 0.47 | 0.72 | 5.24 | 0.0095 | 0.0044 | 0.0176 |
| 3959453 | 0.64 | 0.0075 | 1.41 | 0.6 | 0.7956 | 0.62 | 0.6 | 0.47 | 0.73 | 6.95 | 0.0154 | 0.0054 | 0.0077 |
| 3960634 | 0.69 | 0.0005 | 1.63 | 0.65 | 0.3949 | 0.68 | 0.63 | 0.52 | 0.78 | 5.83 | 0.0003 | 0.0004 | 0.0006 |
| 3961482 | 0.66 | 0.0026 | 1.4 | 0.61 | 0.7405 | 0.6 | 0.62 | 0.47 | 0.73 | 5.74 | 0.0085 | 0.0017 | 0.0028 |
| 3962268 | 0.66 | 0.0024 | 1.7 | 0.63 | 0.611 | 0.68 | 0.6 | 0.49 | 0.77 | 6.13 | 0.0022 | 0.0012 | 0.0019 |
| 3962596 | 0.68 | 0.0007 | 1.44 | 0.59 | 0.8822 | 0.62 | 0.57 | 0.45 | 0.72 | 5.26 | 0.0335 | 0.0009 | 0.0017 |
| 3969510 | 0.6 | 0.0592 | 1.18 | 0.63 | 0.611 | 0.38 | 0.77 | 0.49 | 0.68 | 4.28 | 0.0539 | 0.0442 | 0.062 |
| 3969511 | 0.62 | 0.0292 | 1.69 | 0.65 | 0.3949 | 0.62 | 0.67 | 0.52 | 0.75 | 5.24 | 0.0011 | 0.0074 | 0.0095 |
| 3970894 | 0.7 | 0.0002 | 1.38 | 0.67 | 0.2627 | 0.74 | 0.62 | 0.53 | 0.81 | 7.84 | 0.0001 | 0.0002 | 0.0003 |
| 3974757 | 0.63 | 0.018 | 1.25 | 0.57 | 0.9388 | 0.53 | 0.6 | 0.43 | 0.69 | 4.61 | 0.1211 | 0.0158 | 0.0363 |
| 3974789 | 0.65 | 0.0052 | 1.48 | 0.65 | 0.3949 | 0.64 | 0.66 | 0.52 | 0.76 | 5.33 | 0.0007 | 0.0037 | 0.0049 |
| 3981154 | 0.65 | 0.0044 | 1.57 | 0.64 | 0.4669 | 0.55 | 0.7 | 0.51 | 0.73 | 4.29 | 0.0038 | 0.0024 | 0.0047 |
| 3981752 | 0.61 | 0.0425 | 1.2 | 0.6 | 0.8429 | 0.49 | 0.66 | 0.45 | 0.69 | 5.23 | 0.0726 | 0.0207 | 0.0322 |
| 3981906 | 0.66 | 0.0028 | 1.38 | 0.67 | 0.2627 | 0.66 | 0.67 | 0.53 | 0.77 | 6.5 | 0.0002 | 0.0017 | 0.0024 |
| 3982421 | 0.62 | 0.0239 | 1.42 | 0.66 | 0.3262 | 0.66 | 0.66 | 0.53 | 0.77 | 5.88 | 0.0006 | 0.0114 | 0.0152 |
| 3985558 | 0.65 | 0.0056 | 1.35 | 0.65 | 0.3949 | 0.68 | 0.63 | 0.52 | 0.78 | 7.64 | 0.0003 | 0.0039 | 0.0055 |
| 3985635 | 0.69 | 0.0004 | 1.66 | 0.67 | 0.2627 | 0.68 | 0.66 | 0.53 | 0.78 | 4.81 | 0.0002 | 0.0007 | 0.0009 |
| 3986857 | 0.7 | 0.0002 | 1.48 | 0.66 | 0.3262 | 0.55 | 0.72 | 0.53 | 0.74 | 5.55 | 0.0029 | 0.0002 | 0.0004 |
| 3987532 | 0.62 | 0.0252 | 1.57 | 0.63 | 0.611 | 0.62 | 0.63 | 0.49 | 0.74 | 4.64 | 0.0043 | 0.0175 | 0.019 |
| 3988214 | 0.63 | 0.011 | 1.35 | 0.6 | 0.8429 | 0.62 | 0.59 | 0.46 | 0.73 | 5.28 | 0.0238 | 0.0204 | 0.0239 |
| 3989205 | 0.64 | 0.0069 | 1.36 | 0.64 | 0.5397 | 0.74 | 0.57 | 0.5 | 0.8 | 6.93 | 0.0005 | 0.0029 | 0.0042 |
| 3989770 | 0.69 | 0.0003 | 1.41 | 0.69 | 0.1164 | 0.6 | 0.74 | 0.57 | 0.76 | 3.58 | 0.0001 | 0.0006 | 0.001 |
| 3991722 | 0.68 | 0.0009 | 1.65 | 0.69 | 0.1164 | 0.64 | 0.72 | 0.57 | 0.78 | 4.63 | 0 | 0.0019 | 0.0021 |
| 3993347 | 0.64 | 0.0064 | 1.33 | 0.63 | 0.611 | 0.66 | 0.61 | 0.49 | 0.76 | 7.08 | 0.0023 | 0.0058 | 0.008 |
| 3993349 | 0.65 | 0.0058 | 1.34 | 0.6 | 0.7956 | 0.72 | 0.54 | 0.47 | 0.77 | 6.93 | 0.0038 | 0.0039 | 0.007 |
| 4004822 | 0.69 | 0.0003 | 1.29 | 0.65 | 0.3949 | 0.68 | 0.63 | 0.52 | 0.78 | 4.9 | 0.0005 | 0.0005 | 0.0009 |
| 4007593 | 0.62 | 0.0245 | 1.39 | 0.6 | 0.8429 | 0.55 | 0.62 | 0.46 | 0.71 | 5.8 | 0.0372 | 0.0264 | 0.0282 |
| 4009318 | 0.64 | 0.0064 | 1.33 | 0.63 | 0.611 | 0.77 | 0.55 | 0.49 | 0.8 | 7.87 | 0.0008 | 0.0037 | 0.0039 |
| 4009451 | 0.62 | 0.0265 | 1.28 | 0.64 | 0.4669 | 0.62 | 0.66 | 0.51 | 0.75 | 4.69 | 0.0012 | 0.0174 | 0.0331 |
| 4011604 | 0.63 | 0.0141 | 1.35 | 0.6 | 0.8429 | 0.68 | 0.55 | 0.46 | 0.75 | 5.87 | 0.0106 | 0.0075 | 0.0114 |
| 4011967 | 0.63 | 0.0151 | 1.27 | 0.64 | 0.4669 | 0.64 | 0.65 | 0.51 | 0.76 | 6.49 | 0.001 | 0.0109 | 0.0171 |
| 4012169 | 0.64 | 0.0073 | 1.29 | 0.67 | 0.206 | 0.6 | 0.72 | 0.55 | 0.76 | 6.4 | 0.0004 | 0.0052 | 0.0081 |
| 4012627 | 0.68 | 0.0008 | 1.39 | 0.68 | 0.1571 | 0.64 | 0.71 | 0.56 | 0.77 | 6.08 | 0.0001 | 0.0006 | 0.0009 |
| 4012632 | 0.61 | 0.0391 | 1.33 | 0.63 | 0.611 | 0.51 | 0.7 | 0.49 | 0.71 | 4.5 | 0.0112 | 0.0213 | 0.0316 |
| 4012759 | 0.64 | 0.007 | 1.56 | 0.64 | 0.5397 | 0.57 | 0.67 | 0.5 | 0.73 | 4.05 | 0.0056 | 0.0127 | 0.0139 |
| 4013274 | 0.65 | 0.0041 | 1.4 | 0.66 | 0.3262 | 0.57 | 0.71 | 0.53 | 0.74 | 4.21 | 0.0009 | 0.0074 | 0.0108 |
| 4013282 | 0.69 | 0.0004 | 1.52 | 0.65 | 0.3949 | 0.7 | 0.62 | 0.52 | 0.78 | 5.6 | 0.0004 | 0.0003 | 0.0005 |
| 4015535 | 0.64 | 0.0076 | 1.29 | 0.65 | 0.3949 | 0.6 | 0.68 | 0.52 | 0.75 | 5.28 | 0.0011 | 0.0035 | 0.0091 |
| 4016549 | 0.66 | 0.0031 | 1.48 | 0.67 | 0.2627 | 0.62 | 0.7 | 0.54 | 0.76 | 4.89 | 0.0004 | 0.0045 | 0.0074 |
| 4016573 | 0.66 | 0.0032 | 1.32 | 0.64 | 0.4669 | 0.64 | 0.65 | 0.51 | 0.76 | 7.61 | 0.0019 | 0.0023 | 0.0031 |
| 4019367 | 0.64 | 0.008 | 1.33 | 0.64 | 0.5397 | 0.66 | 0.62 | 0.5 | 0.76 | 5.5 | 0.0012 | 0.0038 | 0.0084 |
| 4019418 | 0.66 | 0.0024 | 1.26 | 0.64 | 0.5397 | 0.57 | 0.67 | 0.5 | 0.73 | 5.56 | 0.0056 | 0.0078 | 0.0058 |
| 4019610 | 0.64 | 0.0078 | 1.37 | 0.62 | 0.6786 | 0.74 | 0.55 | 0.49 | 0.79 | 5.86 | 0.0016 | 0.0072 | 0.0077 |
| 4020462 | 0.67 | 0.0012 | 1.26 | 0.63 | 0.611 | 0.36 | 0.78 | 0.49 | 0.68 | 5 | 0.0833 | 0.0045 | 0.0047 |
| 4024375 | 0.65 | 0.0051 | 1.48 | 0.62 | 0.6786 | 0.68 | 0.59 | 0.48 | 0.76 | 5.36 | 0.0037 | 0.0039 | 0.0058 |
| 4024376 | 0.64 | 0.0109 | 1.54 | 0.64 | 0.4669 | 0.6 | 0.67 | 0.51 | 0.74 | 5.29 | 0.0018 | 0.0106 | 0.0159 |
| 4024377 | 0.63 | 0.0141 | 1.31 | 0.6 | 0.8429 | 0.64 | 0.57 | 0.46 | 0.73 | 6.91 | 0.0175 | 0.0103 | 0.0137 |
| 4024378 | 0.62 | 0.0208 | 1.61 | 0.61 | 0.7405 | 0.57 | 0.63 | 0.47 | 0.72 | 5.3 | 0.0198 | 0.0133 | 0.0213 |
| 4024379 | 0.65 | 0.0039 | 1.49 | 0.64 | 0.4669 | 0.68 | 0.62 | 0.51 | 0.77 | 6.23 | 0.0008 | 0.0025 | 0.0038 |
| 4030986 | 0.7 | 0.0002 | 1.48 | 0.69 | 0.1164 | 0.6 | 0.74 | 0.57 | 0.76 | 5.29 | 0.0001 | 0.002 | 0.0004 |
| 4034997 | 0.65 | 0.0056 | 1.39 | 0.66 | 0.3262 | 0.55 | 0.72 | 0.53 | 0.74 | 4.44 | 0.0019 | 0.0082 | 0.0113 |
| 4035834 | 0.64 | 0.0097 | 1.3 | 0.54 | 0.9881 | 0.66 | 0.48 | 0.42 | 0.71 | 6.32 | 0.1411 | 0.0154 | 0.016 |
| 4035835 | 0.67 | 0.0012 | 1.4 | 0.67 | 0.206 | 0.6 | 0.72 | 0.55 | 0.76 | 4.21 | 0.0003 | 0.0001 | 0.0007 |
| 4035838 | 0.63 | 0.0166 | 1.27 | 0.61 | 0.4669 | 0.51 | 0.72 | 0.51 | 0.72 | 7.03 | 0.0099 | 0.011 | 0.014 |
| 4035839 | 0.65 | 0.0058 | 1.31 | 0.63 | 0.611 | 0.57 | 0.66 | 0.49 | 0.73 | 6.89 | 0.0109 | 0.0056 | 0.0068 |
| 4040526 | 0.67 | 0.0013 | 1.37 | 0.62 | 0.6786 | 0.7 | 0.57 | 0.49 | 0.77 | 7.07 | 0.002 | 0.0013 | 0.0015 |
| 4041367 | 0.63 | 0.0147 | 1.28 | 0.58 | 0.914 | 0.51 | 0.62 | 0.44 | 0.69 | 4.36 | 0.151 | 0.0166 | 0.0219 |
| 4041822 | 0.65 | 0.0044 | 1.29 | 0.58 | 0.914 | 0.77 | 0.48 | 0.46 | 0.78 | 5.7 | 0.0066 | 0.0037 | 0.0051 |
| 4043125 | 0.65 | 0.0051 | 1.29 | 0.61 | 0.7405 | 0.55 | 0.65 | 0.47 | 0.72 | 4.38 | 0.0262 | 0.0108 | 0.0129 |
| 4044199 | 0.65 | 0.0056 | 1.22 | 0.64 | 0.4669 | 0.55 | 0.7 | 0.51 | 0.73 | 6.37 | 0.0034 | 0.0044 | 0.0069 |
| 4044413 | 0.63 | 0.0121 | 1.24 | 0.63 | 0.611 | 0.47 | 0.72 | 0.49 | 0.7 | 4.71 | 0.0241 | 0.0129 | 0.0129 |
| 4047577 | 0.63 | 0.0121 | 1.4 | 0.64 | 0.5397 | 0.74 | 0.57 | 0.5 | 0.8 | 5.82 | 0.0006 | 0.0086 | 0.0074 |
| 4052399 | 0.69 | 0.0003 | 1.46 | 0.69 | 0.1164 | 0.6 | 0.74 | 0.57 | 0.76 | 5.7 | 0.0001 | 0.0005 | 0.0008 |
| 4055295 | 0.7 | 0.0001 | 1.45 | 0.64 | 0.5397 | 0.62 | 0.65 | 0.5 | 0.75 | 5.94 | 0.0031 | 0.0002 | 0.0002 |
| 4055302 | 0.69 | 0.0004 | 1.29 | 0.69 | 0.1164 | 0.74 | 0.66 | 0.56 | 0.82 | 4.81 | 0 | 0.0005 | 0.001 |
| 2720180 | 0.61 | 0.0307 | 1.74 | 0.61 | 0.7405 | 0.66 | 0.59 | 0.48 | 0.75 | 4.96 | 0.0116 | 0.0244 | 0.0176 |
| 2347642 | 0.71 | 0.0001 | 1.11 | 0.69 | 0.1164 | 0.45 | 0.83 | 0.6 | 0.72 | 6.16 | 0.0001 | 0 | 0.0007 |
| 2395344 | 0.73 | 0 | 1.2 | 0.66 | 0.3262 | 0.83 | 0.56 | 0.52 | 0.85 | 4.69 | 0 | 0 | 0.0001 |
| 2397069 | 0.7 | 0.0002 | 1.15 | 0.69 | 0.1164 | 0.64 | 0.72 | 0.57 | 0.78 | 6.11 | 0 | 0 | 0.0002 |
| 2430377 | 0.63 | 0.0151 | 1.07 | 0.61 | 0.7405 | 0.53 | 0.66 | 0.4 | 0.71 | 5.43 | 0.034 | 0.0319 | 0.0413 |
| 2448270 | 0.68 | 0.0007 | 1.08 | 0.61 | 0.5397 | 0.62 | 0.65 | 0.5 | 0.75 | 6.9 | 0.005 | 0.0017 | 0.0021 |
| 2553581 | 0.67 | 0.0012 | 1.14 | 0.62 | 0.6786 | 0.66 | 0.6 | 0.48 | 0.75 | 4.92 | 0.0057 | 0.0005 | 0.0017 |
| 2600700 | 0.74 | 0 | 1.16 | 0.65 | 0.3949 | 0.72 | 0.61 | 0.52 | 0.79 | 4.7 | 0.0006 | 0 | 0.0001 |
| 2624519 | 0.7 | 0.0002 | 1.12 | 0.61 | 0.7405 | 0.72 | 0.55 | 0.48 | 0.78 | 4.56 | 0.003 | 0.0005 | 0.0007 |
| 2651846 | 0.67 | 0.0011 | 1.16 | 0.64 | 0.5397 | 0.66 | 0.62 | 0.5 | 0.76 | 3.02 | 0.001 | 0.0009 | 0.0024 |
| 2682441 | 0.69 | 0.0004 | 1.14 | 0.62 | 0.6786 | 0.74 | 0.55 | 0.49 | 0.79 | 5.91 | 0.001 | 0.0006 | 0.0015 |
| 2704512 | 0.74 | 0 | 1.16 | 0.66 | 0.3262 | 0.77 | 0.6 | 0.52 | 0.82 | 4.33 | 0.0001 | 0 | 0 |
| 2704702 | 0.74 | 0 | 1.11 | 0.64 | 0.4669 | 0.74 | 0.59 | 0.51 | 0.8 | 7.7 | 0.0004 | 0 | 0.0001 |
| 2902832 | 0.74 | 0 | 0.96 | 0.33 | 1 | 0.51 | 0.22 | 0.27 | 0.44 | 6.72 | 0.001 | 0 | 0.0001 |
| 2918558 | 0.71 | 0.0001 | 1.19 | 0.67 | 0.2627 | 0.4 | 0.82 | 0.56 | 0.71 | 5.01 | 0.0065 | 0.0005 | 0.0005 |
| 3031661 | 0.76 | 0 | 0.94 | 0.25 | 1 | 0.47 | 0.12 | 0.23 | 0.29 | 6.09 | 0 | 0 | 0 |
| 3212761 | 0.77 | 0 | 1.24 | 0.7 | 0.0837 | 0.77 | 0.66 | 0.56 | 0.83 | 4.25 | 0 | 0 | 0 |
| 3268183 | 0.66 | 0.0031 | 1.04 | 0.63 | 0.611 | 0.66 | 0.61 | 0.49 | 0.76 | 6.74 | 0.003 | 0.0036 | 0.0055 |
| 3472753 | 0.71 | 0.0001 | 0.94 | 0.33 | 1 | 0.36 | 0.32 | 0.23 | 0.46 | 7.02 | 0.0004 | 0.0002 | 0.0005 |
| 3501557 | 0.7 | 0.0002 | 1.22 | 0.66 | 0.3262 | 0.6 | 0.7 | 0.53 | 0.75 | 4.53 | 0.0023 | 0.0004 | 0.0005 |
| 3543621 | 0.65 | 0.0039 | 0.94 | 0.36 | 1 | 0.53 | 0.26 | 0.29 | 0.49 | 5.01 | 0.0111 | 0.0171 | 0.0139 |
| 3541113 | 0.7 | 0.0002 | 1.12 | 0.67 | 0.2627 | 0.6 | 0.71 | 0.54 | 0.75 | 6.09 | 0.0008 | 0.0003 | 0.0004 |
| 3562041 | 0.71 | 0.0001 | 1.27 | 0.7 | 0.0837 | 0.64 | 0.73 | 0.58 | 0.78 | 6.32 | 0 | 0.0002 | 0.0004 |
| 3661099 | 0.67 | 0.0016 | 1.0 | 0.66 | 0.3262 | 0.57 | 0.71 | 0.53 | 0.74 | 6.54 | 0.0022 | 0.0022 | 0.004 |
| 3667907 | 0.7 | 0.0002 | 1.06 | 0.63 | 0.611 | 0.6 | 0.65 | 0.49 | 0.74 | 7.88 | 0.0044 | 0.0003 | 0.0005 |
| 3701840 | 0.65 | 0.0042 | 1.1 | 0.64 | 0.4669 | 0.51 | 0.72 | 0.51 | 0.72 | 5.52 | 0.0072 | 0.0019 | 0.0036 |
| 3840609 | 0.66 | 0.0029 | 1.19 | 0.67 | 0.2627 | 0.4 | 0.82 | 0.56 | 0.71 | 3.06 | 0.0048 | 0.0042 | 0.0072 |
| 3889144 | 0.71 | 0.0001 | 1.13 | 0.64 | 0.4669 | 0.68 | 0.62 | 0.51 | 0.77 | 5.67 | 0.0004 | 0 | 0.0002 |
| 3889625 | 0.73 | 0 | 1.21 | 0.66 | 0.3262 | 0.66 | 0.66 | 0.53 | 0.77 | 5.21 | 0.0004 | 0 | 0 |
| 3897081 | 0.71 | 0.0001 | 1.15 | 0.71 | 0.0394 | 0.55 | 0.8 | 0.62 | 0.76 | 5.7 | 0 | 0.0002 | 0.0006 |
| 3902441 | 0.67 | 0.001 | 0.95 | 0.35 | 1 | 0.38 | 0.33 | 0.25 | 0.48 | 6.27 | 0.0007 | 0.0031 | 0.0047 |
| 4026042 | 0.66 | 0.0027 | 1.16 | 0.65 | 0.3949 | 0.55 | 0.71 | 0.52 | 0.73 | 7.94 | 0.0016 | 0.0004 | 0.0014 |

Performance of the 1,425 detected biomarkers across different metrics for the recurrence endpoint on the subset of patients with LNI negative (n=129). See Example 2 for explanation of metrics.

TABLE 20

| | | | | | | | | | | | | UVA | UVA |
|---|---|---|---|---|---|---|---|---|---|---|---|---|---|
| | | WILCOX | | | | | | | | | | HR | OR |
| | | P- | KS P- | T-TEST | | | | | CUT | KM P- | SURV | P- | P- |
| CLASSIFIER | AUC | VALUE | VALUE | P-VALUE | SENSITIVITY | SPECIFICITY | PPV | NPV | OFF | VALUE | AUC | VALUE | VALUE |
| nb: 3169011 | 0.94 | 0.0003 | 0.0002 | 0 | 0.89 | 0.86 | 0.94 | 0.75 | 0.48 | 0.001 | 0.92 | 0.003 | 0.007 |
| 3889144 | | | | | | | | | | | | | |
| knn: 3169011 | 0.92 | 0.0013 | 0.0045 | 0.0001 | 0.83 | 0.86 | 0.94 | 0.67 | 0.53 | 0.0103 | 0.9 | 0.001 | 0.009 |
| 3840609 | | | | | | | | | | | | | |
| rf: 3169011 | 0.92 | 0.0015 | 0.0007 | 0.0001 | 0.89 | 0.86 | 0.94 | 0.75 | 0.27 | 0.0013 | 0.94 | 0.004 | 0.01 |
| 3889144 | | | | | | | | | | | | | |
| nb: 3169011 | 0.92 | 0.0005 | 0.0001 | 0.0005 | 0.78 | 1 | 1 | 0.64 | 0.54 | 0 | 0.89 | 0.002 | 0.011 |
| 2553581 | | | | | | | | | | | | | |
| svm: 3169011 | 0.91 | 0.0008 | 0.0006 | 0.0003 | 0.72 | 1 | 1 | 0.58 | 0.39 | 0.0046 | 0.91 | 0.015 | 0.012 |
| 3889625 | | | | | | | | | | | | | |
| nb: 3169011 | 0.87 | 0.003 | 0.0083 | 0.0016 | 0.83 | 0.86 | 0.94 | 0.67 | 0.54 | 0.0013 | 0.89 | 0.005 | 0.012 |
| 3969511 | | | | | | | | | | | | | |
| knn: 3562041 | 0.87 | 0.0055 | 0.0464 | 0.0027 | 0.89 | 0.57 | 0.84 | 0.67 | 0.37 | 0.0167 | 0.85 | 0.006 | 0.012 |
| 2397069 | | | | | | | | | | | | | |
| knn: 3969511 | 0.82 | 0.0049 | 0.0344 | 0.0023 | 0.78 | 0.86 | 0.93 | 0.6 | 0.5 | 0.0409 | 0.82 | 0.049 | 0.013 |
| 3962268 | | | | | | | | | | | | | |
| nb: 3169011 | 0.87 | 0.0039 | 0.0018 | 0.0019 | 0.78 | 1 | 1 | 0.64 | 0.53 | 0.0005 | 0.87 | 0.003 | 0.013 |
| 3151480 | | | | | | | | | | | | | |
| svm: 3169011 | 0.89 | 0.0018 | 0.0083 | 0.0006 | 0.72 | 0.86 | 0.93 | 0.55 | 0.23 | 0.0041 | 0.9 | 0.008 | 0.013 |
| 3661099 | | | | | | | | | | | | | |
| rf: 3169011 | 0.86 | 0.0049 | 0.003 | 0.0012 | 0.89 | 0.86 | 0.94 | 0.75 | 0.47 | 0.001 | 0.89 | 0.003 | 0.013 |
| 2397069 | | | | | | | | | | | | | |
| nb: 3169011 | 0.87 | 0.003 | 0.0048 | 0.002 | 0.72 | 0.86 | 0.93 | 0.55 | 0.56 | 0.0038 | 0.86 | 0.007 | 0.013 |
| 3498523 | | | | | | | | | | | | | |
| rf: 3169011 | 0.87 | 0.0053 | 0.0227 | 0.0019 | 0.72 | 0.86 | 0.93 | 0.55 | 0.6 | 0.0301 | 0.87 | 0.02 | 0.013 |
| 3889625 | | | | | | | | | | | | | |
| nb: 3169011 | 0.9 | 0.0014 | 0.0048 | 0.0018 | 0.72 | 1 | 1 | 0.58 | 0.54 | 0.0002 | 0.87 | 0.004 | 0.013 |
| 3212761 | | | | | | | | | | | | | |
| nb: 3169011 | 0.87 | 0.003 | 0.0048 | 0.0021 | 0.72 | 0.86 | 0.93 | 0.55 | 0.53 | 0.0038 | 0.86 | 0.007 | 0.014 |
| 3872197 | | | | | | | | | | | | | |
| knn: 3169011 | 0.9 | 0.0021 | 0.0073 | 0.0005 | 0.78 | 0.86 | 0.93 | 0.6 | 0.52 | 0.0207 | 0.87 | 0.014 | 0.014 |
| 3661099 | | | | | | | | | | | | | |
| nb: 3169011 | 0.87 | 0.0039 | 0.0048 | 0.0035 | 0.72 | 0.86 | 0.93 | 0.55 | 0.54 | 0.0038 | 0.86 | 0.005 | 0.014 |
| 3831128 | | | | | | | | | | | | | |
| svm: 3169011 | 0.93 | 0.0004 | 0.0006 | 0.0001 | 0.67 | 1 | 1 | 0.54 | 0.55 | 0.0017 | 0.92 | 0.001 | 0.015 |
| 3840609 | | | | | | | | | | | | | |
| nb: 3169011 | 0.87 | 0.0039 | 0.0018 | 0.0015 | 0.78 | 1 | 1 | 0.64 | 0.57 | 0.0005 | 0.88 | 0.005 | 0.015 |
| 3501555 | | | | | | | | | | | | | |
| svm: 3562041 | 0.82 | 0.0141 | 0.0325 | 0.0028 | 0.72 | 0.86 | 0.93 | 0.55 | 0.24 | 0.0231 | 0.83 | 0.03 | 0.015 |
| 3969511 | | | | | | | | | | | | | |
| svm: 3169011 | 0.92 | 0.0005 | 0.0001 | 0.0005 | 0.61 | 1 | 1 | 0.5 | 0.46 | 0.0023 | 0.91 | 0.002 | 0.015 |
| 2553581 | | | | | | | | | | | | | |
| svm: 3169011 | 0.94 | 0.0003 | 0.0001 | 0.0001 | 0.78 | 1 | 1 | 0.64 | 0.32 | 0.0005 | 0.93 | 0.001 | 0.015 |
| 3562041 | | | | | | | | | | | | | |
| nb: 3169011 | 0.89 | 0.0018 | 0.0006 | 0.0014 | 0.72 | 1 | 1 | 0.58 | 0.55 | 0.0002 | 0.85 | 0.004 | 0.015 |
| 3962268 | | | | | | | | | | | | | |
| svm: 3169011 | 0.87 | 0.0039 | 0.0018 | 0.0021 | 0.72 | 1 | 1 | 0.58 | 0.32 | 0.0002 | 0.86 | 0.004 | 0.016 |
| 3151480 | | | | | | | | | | | | | |
| rf: 3169011 | 0.84 | 0.011 | 0.0104 | 0.0035 | 0.78 | 0.86 | 0.93 | 0.6 | 0.53 | 0.0054 | 0.85 | 0.008 | 0.016 |
| 3501555 | | | | | | | | | | | | | |
| nb: 3169011 | 0.84 | 0.0076 | 0.0048 | 0.0029 | 0.72 | 1 | 1 | 0.58 | 0.55 | 0.0002 | 0.85 | 0.005 | 0.016 |
| 2553585 | | | | | | | | | | | | | |
| svm: 3169011 | 0.9 | 0.0014 | 0.003 | 0.0002 | 0.72 | 0.86 | 0.93 | 0.55 | 0.38 | 0.0486 | 0.91 | 0.014 | 0.017 |
| 3551833 | | | | | | | | | | | | | |
| svm: 3169011 | 0.87 | 0.0039 | 0.0018 | 0.0012 | 0.78 | 1 | 1 | 0.64 | 0.07 | 0.0005 | 0.86 | 0.004 | 0.017 |
| 3501555 | | | | | | | | | | | | | |
| rf: 3562041 | 0.79 | 0.0316 | 0.0511 | 0.0052 | 0.89 | 0.71 | 0.89 | 0.71 | 0.44 | 0.0031 | 0.81 | 0.022 | 0.017 |
| 3719123 | | | | | | | | | | | | | |
| nb: 3169011 | 0.84 | 0.0076 | 0.0018 | 0.0041 | 0.78 | 1 | 1 | 0.64 | 0.53 | 0.0005 | 0.85 | 0.005 | 0.018 |
| 3666869 | | | | | | | | | | | | | |
| nb: 3169011 | 0.9 | 0.001 | 0.0083 | 0.0007 | 0.61 | 1 | 1 | 0.5 | 0.61 | 0.0055 | 0.88 | 0.003 | 0.018 |
| 3551833 | | | | | | | | | | | | | |
| knn: 3169011 | 0.85 | 0.0075 | 0.0344 | 0.0035 | 0.78 | 0.86 | 0.93 | 0.6 | 0.53 | 0.0054 | 0.85 | 0.007 | 0.018 |
| 3501555 | | | | | | | | | | | | | |
| svm: 3169011 | 0.87 | 0.003 | 0.0018 | 0.0008 | 0.72 | 1 | 1 | 0.58 | 0.26 | 0.0002 | 0.88 | 0.003 | 0.018 |
| 3962268 | | | | | | | | | | | | | |
| nb: 3169011 | 0.85 | 0.0061 | 0.0202 | 0.0037 | 0.72 | 0.86 | 0.93 | 0.55 | 0.52 | 0.0041 | 0.86 | 0.009 | 0.018 |
| 3661099 | | | | | | | | | | | | | |
| knn: 3169011 | 0.88 | 0.0043 | 0.0104 | 0.0025 | 0.89 | 0.57 | 0.84 | 0.67 | 0.47 | 0.0159 | 0.87 | 0.011 | 0.018 |
| 2553581 | | | | | | | | | | | | | |
| svm: 2553581 | 0.79 | 0.0245 | 0.0431 | 0.0044 | 0.89 | 0.57 | 0.84 | 0.67 | −0.02 | 0.026 | 0.79 | 0.038 | 0.018 |
| 3562041 | | | | | | | | | | | | | |
| svm: 3562041 | 0.83 | 0.0116 | 0.0325 | 0.0059 | 0.83 | 0.71 | 0.88 | 0.63 | −0.37 | 0.009 | 0.83 | 0.022 | 0.019 |
| 2397069 | | | | | | | | | | | | | |
| nb: 3169011 | 0.83 | 0.0094 | 0.0048 | 0.0064 | 0.72 | 0.71 | 0.87 | 0.5 | 0.51 | 0.0266 | 0.82 | 0.009 | 0.019 |
| 3719123 | | | | | | | | | | | | | |
| nb: 3169011 | 0.93 | 0.0004 | 0.0006 | 0.0003 | 0.61 | 1 | 1 | 0.5 | 0.64 | 0.0004 | 0.91 | 0.001 | 0.02 |
| 3840609 | | | | | | | | | | | | | |
| rf: 3169011 | 0.89 | 0.0033 | 0.0104 | 0.001 | 0.67 | 1 | 1 | 0.54 | 0.66 | 0.0014 | 0.88 | 0.004 | 0.02 |
| 3969511 | | | | | | | | | | | | | |
| nb: 3169011 | 0.94 | 0.0003 | 0.0006 | 0.0001 | 0.78 | 1 | 1 | 0.64 | 0.56 | 0.0005 | 0.92 | 0 | 0.02 |
| 3562041 | | | | | | | | | | | | | |
| rf: 3169011 | 0.86 | 0.0064 | 0.0227 | 0.002 | 0.61 | 1 | 1 | 0.5 | 0.66 | 0.0001 | 0.88 | 0.001 | 0.021 |
| 3840609 | | | | | | | | | | | | | |
| svm: 3169011 | 0.85 | 0.0061 | 0.0048 | 0.0038 | 0.67 | 1 | 1 | 0.54 | 0.42 | 0.0006 | 0.85 | 0.007 | 0.021 |
| 2553585 | | | | | | | | | | | | | |
| rf: 3169011 | 0.9 | 0.0022 | 0.0164 | 0.0001 | 0.67 | 1 | 1 | 0.54 | 0.69 | 0.0013 | 0.89 | 0.003 | 0.021 |
| 3661099 | | | | | | | | | | | | | |
| nb: 3169011 | 0.81 | 0.0171 | 0.0325 | 0.0085 | 0.83 | 0.71 | 0.88 | 0.63 | 0.47 | 0.0092 | 0.8 | 0.013 | 0.022 |
| 3031661 | | | | | | | | | | | | | |
| nb: 3562041 | 0.82 | 0.0141 | 0.0684 | 0.0084 | 0.78 | 0.71 | 0.88 | 0.56 | 0.5 | 0.0379 | 0.8 | 0.037 | 0.022 |
| 3840609 | | | | | | | | | | | | | |
| nb: 3169011 | 0.83 | 0.0094 | 0.0048 | 0.0058 | 0.72 | 1 | 1 | 0.58 | 0.56 | 0.0119 | 0.82 | 0.043 | 0.022 |
| 2624519 | | | | | | | | | | | | | |
| svm: 3169011 | 0.81 | 0.0171 | 0.0325 | 0.0076 | 0.83 | 0.71 | 0.88 | 0.63 | −0.12 | 0.0092 | 0.81 | 0.011 | 0.022 |
| 3031661 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 2553581 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3562041 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3661099 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3889625 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3151480 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3872197 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3719123 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3498523 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3969511 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3962268 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3831128 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 2624519 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3212761 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 3501555 | | | | | | | | | | | | | |
| rpart: 3169011 | 0.79 | 0.0113 | 0.0679 | 0.0072 | 0.72 | 0.86 | 0.93 | 0.55 | 0.48 | 0.0038 | 0.79 | 0.008 | 0.022 |
| 2600700 | | | | | | | | | | | | | |
| knn: 3169011 | 0.87 | 0.0044 | 0.0744 | 0.0013 | 0.5 | 1 | 1 | 0.44 | 0.61 | 0.0153 | 0.87 | 0.016 | 0.022 |
| 3551833 | | | | | | | | | | | | | |
| rf: 3169011 | 0.84 | 0.01 | 0.0104 | 0.0085 | 0.72 | 0.71 | 0.87 | 0.5 | 0.45 | 0.0266 | 0.84 | 0.011 | 0.023 |
| 3962268 | | | | | | | | | | | | | |
| rf: 3169011 | 0.92 | 0.0005 | 0.003 | 0.0003 | 0.61 | 1 | 1 | 0.5 | 0.62 | 0 | 0.91 | 0.001 | 0.023 |
| 3562041 | | | | | | | | | | | | | |
| rf: 3169011 | 0.82 | 0.0141 | 0.0431 | 0.0071 | 0.72 | 0.86 | 0.93 | 0.55 | 0.61 | 0.0038 | 0.83 | 0.008 | 0.023 |
| 3498523 | | | | | | | | | | | | | |
| rf: 3169011 | 0.85 | 0.0061 | 0.0018 | 0.0041 | 0.56 | 1 | 1 | 0.47 | 0.64 | 0.0117 | 0.84 | 0.019 | 0.023 |
| 2624519 | | | | | | | | | | | | | |
| rf: 3562041 | 0.77 | 0.0426 | 0.0744 | 0.0055 | 0.78 | 0.71 | 0.88 | 0.56 | 0.61 | 0.0333 | 0.76 | 0.045 | 0.023 |
| 3661099 | | | | | | | | | | | | | |
| nb: 3840609 | 0.83 | 0.0116 | 0.0431 | 0.0068 | 0.78 | 0.71 | 0.88 | 0.56 | 0.53 | 0.0262 | 0.84 | 0.031 | 0.023 |
| 3551833 | | | | | | | | | | | | | |
| rf: 3169011 | 0.83 | 0.012 | 0.0227 | 0.0062 | 0.72 | 0.86 | 0.93 | 0.55 | 0.46 | 0.0038 | 0.82 | 0.006 | 0.023 |
| 3719123 | | | | | | | | | | | | | |
| rf: 3169011 | 0.85 | 0.0084 | 0.0045 | 0.0051 | 0.67 | 1 | 1 | 0.54 | 0.51 | 0.0005 | 0.83 | 0.019 | 0.024 |
| 2600700 | | | | | | | | | | | | | |
| rf: 3169011 | 0.79 | 0.0316 | 0.0464 | 0.0104 | 0.56 | 1 | 1 | 0.47 | 0.71 | 0.0084 | 0.81 | 0.014 | 0.026 |
| 3031661 | | | | | | | | | | | | | |
| svm: 3889144 | 0.87 | 0.0039 | 0.0006 | 0.0023 | 0.72 | 1 | 1 | 0.58 | −0.13 | 0.0207 | 0.87 | 0.034 | 0.026 |
| 3661099 | | | | | | | | | | | | | |
| nb: 3169011 | 0.81 | 0.0171 | 0.0083 | 0.0081 | 0.61 | 0.86 | 0.92 | 0.46 | 0.58 | 0.0037 | 0.82 | 0.006 | 0.026 |
| 2397069 | | | | | | | | | | | | | |
| rf: 3169011 | 0.9 | 0.0025 | 0.0018 | 0.0023 | 0.5 | 1 | 1 | 0.44 | 0.66 | 0.0022 | 0.89 | 0.003 | 0.026 |
| 2553581 | | | | | | | | | | | | | |
| svm: 3169011 | 0.9 | 0.001 | 0.0048 | 0.0007 | 0.67 | 1 | 1 | 0.54 | 0.18 | 0.0004 | 0.9 | 0.003 | 0.027 |
| 3212761 | | | | | | | | | | | | | |
| rpart: 3562436 | 0.73 | 0.0198 | 0.2359 | 0.0142 | 0.89 | 0.57 | 0.84 | 0.67 | 0.46 | 0.0167 | 0.73 | 0.03 | 0.027 |
| 3562041 | | | | | | | | | | | | | |
| rpart: 2553581 | 0.73 | 0.0198 | 0.2359 | 0.0142 | 0.89 | 0.57 | 0.84 | 0.67 | 0.46 | 0.0167 | 0.73 | 0.03 | 0.027 |
| 3562041 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3661099 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3551833 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3151480 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3872197 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 2553585 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3719123 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3498523 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3962268 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3831128 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3666869 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 2624519 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3212761 | | | | | | | | | | | | | |
| rpart: 3562041 | 0.73 | 0.0198 | 0.2359 | 0.0142 | 0.89 | 0.57 | 0.84 | 0.67 | 0.46 | 0.0167 | 0.73 | 0.03 | 0.027 |
| 3501555 | | | | | | | | | | | | | |
| svm: 3169011 | 0.83 | 0.0094 | 0.0118 | 0.0121 | 0.67 | 1 | 1 | 0.54 | 0.2 | 0.0004 | 0.83 | 0.006 | 0.028 |
| 3831128 | | | | | | | | | | | | | |
| svm: 3169011 | 0.86 | 0.0049 | 0.0083 | 0.0043 | 0.61 | 0.86 | 0.92 | 0.46 | 0.5 | 0.0037 | 0.85 | 0.004 | 0.03 |
| 2397069 | | | | | | | | | | | | | |
| svm: 2397069 | 0.8 | 0.0205 | 0.0563 | 0.004 | 0.44 | 1 | 1 | 0.41 | 0.61 | 0.0262 | 0.81 | 0.032 | 0.03 |
| 3212761 | | | | | | | | | | | | | |
| nb: 3562041 | 0.81 | 0.0171 | 0.0264 | 0.0125 | 0.67 | 0.86 | 0.92 | 0.5 | 0.56 | 0.0405 | 0.82 | 0.038 | 0.03 |
| 3661099 | | | | | | | | | | | | | |
| knn: 3169011 | 0.86 | 0.0056 | 0.0104 | 0.0044 | 0.56 | 1 | 1 | 0.47 | 0.64 | 0.0215 | 0.86 | 0.003 | 0.03 |
| 3212761 | | | | | | | | | | | | | |
| nb: 3889144 | 0.87 | 0.0039 | 0.0006 | 0.0072 | 0.78 | 1 | 1 | 0.64 | 0.52 | 0.0066 | 0.87 | 0.047 | 0.031 |
| 3661099 | | | | | | | | | | | | | |
| knn: 2553581 | 0.78 | 0.0318 | 0.0973 | 0.0159 | 0.89 | 0.57 | 0.84 | 0.67 | 0.39 | 0.026 | 0.79 | 0.033 | 0.031 |
| 3562041 | | | | | | | | | | | | | |
| rpart: 3169011 | 0.79 | 0.0147 | 0.0679 | 0.0126 | 0.67 | 0.86 | 0.92 | 0.5 | 0.62 | 0.0109 | 0.79 | 0.014 | 0.032 |
| 3562436 | | | | | | | | | | | | | |
| svm: 3562041 | 0.79 | 0.0245 | 0.0325 | 0.0225 | 0.78 | 0.71 | 0.88 | 0.56 | −0.26 | 0.0288 | 0.8 | 0.031 | 0.033 |
| 3872197 | | | | | | | | | | | | | |
| nb: 3661099 | 0.86 | 0.0049 | 0.0202 | 0.0127 | 0.5 | 1 | 1 | 0.44 | 0.59 | 0.0427 | 0.84 | 0.02 | 0.033 |
| 3031661 | | | | | | | | | | | | | |
| knn: 3169011 | 0.78 | 0.0337 | 0.0511 | 0.0187 | 0.89 | 0.71 | 0.89 | 0.71 | 0.5 | 0.0107 | 0.78 | 0.026 | 0.034 |
| 2397069 | | | | | | | | | | | | | |
| rf: 3169011 | 0.88 | 0.0044 | 0.0045 | 0.0012 | 0.67 | 1 | 1 | 0.54 | 0.56 | 0.0014 | 0.86 | 0.004 | 0.038 |
| 2553585 | | | | | | | | | | | | | |
| knn: 3169011 | 0.83 | 0.0137 | 0.0104 | 0.0166 | 0.72 | 1 | 1 | 0.58 | 0.5 | 0.0016 | 0.82 | 0.027 | 0.04 |
| 3889144 | | | | | | | | | | | | | |
| rf: 3169011 | 0.82 | 0.0141 | 0.0048 | 0.0241 | 0.67 | 1 | 1 | 0.54 | 0.67 | 0.0008 | 0.81 | 0.025 | 0.041 |
| 3562436 | | | | | | | | | | | | | |
| svm: 2397069 | 0.82 | 0.0141 | 0.0563 | 0.007 | 0.5 | 1 | 1 | 0.44 | 0.24 | 0.0451 | 0.8 | 0.032 | 0.043 |
| 3969511 | | | | | | | | | | | | | |
| rpart: 3889144 | 0.85 | 0.0061 | 0.0227 | 0.0023 | 0.67 | 1 | 1 | 0.54 | 0.5 | 0.018 | 0.85 | 0.028 | 0.045 |
| 3889625 | | | | | | | | | | | | | |
| knn: 3889144 | 0.77 | 0.0363 | 0.2191 | 0.0301 | 0.61 | 0.86 | 0.92 | 0.46 | 0.5 | 0.0458 | 0.77 | 0.025 | 0.048 |
| 2397069 | | | | | | | | | | | | | |

Performance across multiple metrics of pairwise combinations of markers selected from the library (Table 16, Table 18) in the subset of patients with positive lymph node involvement.

What is claimed is:

1. A method of diagnosing, prognosing, determining progression of cancer, predicting a therapeutic regimen or predicting benefit from therapy in a subject having cancer, comprising:
(a) assaying an expression level in a sample from the subject for a plurality of targets, wherein the plurality of targets comprises one or more targets selected from Table 2B, Table 16 or SEQ ID NOs:1-1440; and
(b) diagnosing, prognosing, determining progression of cancer, predicting a therapeutic regimen or predicting benefit from therapy in a subject having cancer based on the expression levels of the plurality of targets.

2. The method of claim 1, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

3. The method of claim 1, wherein the cancer is a bladder cancer.

4. The method of claim 1, wherein the plurality of targets comprises a coding target.

5. The method of claim 4, wherein the coding target is an exonic sequence.

6. The method of claim 1, wherein the plurality of targets comprises a non-coding target.

7. The method of claim 6, wherein the non-coding target comprises an intronic sequence or partially overlaps an intronic sequence.

8. The method of claim 6, wherein the non-coding target comprises a sequence within the UTR or partially overlaps with a UTR sequence.

9. The method of any claim 1, wherein the target comprises a nucleic acid sequence.

10. The method of claim 9, wherein the nucleic acid sequence is a DNA sequence.

11. The method of claim 9, wherein the nucleic acid sequence is an RNA sequence.

12. The method of claim 1, wherein the plurality of targets comprises at least 5 targets selected from Table 2B, Table 16 or SEQ ID NOs:1-1440.

13. The method of claim 1, wherein the plurality of targets comprises at least 10 targets selected from Table 2B, Table 16 or SEQ ID NOs:1-1440.

14. The method of claim 1, wherein the plurality of targets comprises at least 15 targets selected from Table 2B, Table 16 or SEQ ID NOs:1-1440.

15. The method of claim 1, wherein the plurality of targets comprises SEQ ID NOs: 1-15.

16. The method of claim 1, wherein the diagnosing, prognosing, determining progression of cancer, predicting therapeutic regimen or predicting benefit from therapy includes assessing the risk of cancer recurrence.

17. The method of claim 1, further comprising sequencing the plurality of targets.

18. The method of claim 1, further comprising hybridizing the plurality of targets to a solid support.

19. The method of claim 18, wherein the solid support is a bead or array.

20. A probe set for assessing a cancer status of a subject comprising a plurality of probes, wherein the probes in the set are capable of detecting an expression level of one or more targets selected from Table 2B, Table 16 or SEQ ID NOs:1-1440, wherein the expression level determines the cancer status of the subject with at least 40% specificity.

21. The probe set of claim 20, wherein the one or more targets comprises SEQ ID NOs: 1-15.

22. The probe set of claim 20, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

23. The probe set of claim 20, wherein the cancer is a bladder cancer.

24. The probe set of claim 20, wherein the probe set further comprises a probe capable of detecting an expression level of at least one coding target.

25. The probe set of claim 24, wherein the coding target is an exonic sequence.

26. The probe set of claim 20, wherein the probe set further comprises a probe capable of detecting an expression level of at least one non-coding target.

27. The probe set of claim 26, wherein the non-coding target is an intronic sequence or partially overlaps with an intronic sequence.

28. The probe set of claim 26, wherein the non-coding target is a UTR sequence or partially overlaps with a UTR sequence.

29. The probe set of claim 20, wherein assessing the cancer status includes assessing cancer recurrence risk.

30. The probe set of claim 20, wherein the target is a nucleic acid sequence.

31. The probe set of claim 30, wherein the nucleic acid sequence is a DNA sequence.

32. The probe set of claim 30, wherein the nucleic acid sequence is an RNA sequence.

33. The probe set of claim 20, wherein the probes are between about 15 nucleotides and about 500 nucleotides in length.

34. The probe set of claim 20, wherein the probes are at least 15 nucleotides in length.

35. The probe set of claim 20, wherein the probes are at least 25 nucleotides in length.

36. The probe set of claim 20, wherein the non-coding target is a non-coding RNA transcript and the non-coding RNA transcript is non-polyadenylated.

37. A system for analyzing a cancer, comprising:
(a) a probe set comprising a plurality of target sequences, wherein
(i) the plurality of target sequences hybridizes to one or more targets selected from Table 2B, Table 16 or SEQ ID NOs:1-1440; or
(ii) the plurality of target sequences comprises one or more target sequences selected from Table 2B, Table 16 or SEQ ID NOs:1-1440; and
(b) a computer model or algorithm for analyzing an expression level and/or expression profile of the target hybridized to the probe in a sample from a subject suffering from a cancer.

38. The system of claim 37, wherein the plurality of target sequences comprises SEQ ID NOs: 1-15

39. The system of claim 37, further comprising a label that specifically binds to the target, the probe, or a combination thereof.

40. The system of claim 37, wherein the plurality of target sequences comprises at least 5 target sequences selected from Table 2B, Table 16 or SEQ ID NOs:1-1440.

41. The system of claim 37, wherein the plurality of target sequences comprises at least 10 target sequences selected from Table 2B, Table 16 or SEQ ID NOs:1-1440.

42. The system of claim 37, wherein the plurality of target sequences comprises at least 15 target sequences selected from Table 2B, Table 16 or SEQ ID NOs:1-1440.

43. The system of claim 37, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

44. A method of analyzing a cancer in an individual in need thereof, comprising:
(a) obtaining an expression profile from a sample obtained from the individual, wherein the expression profile comprises one or more targets selected from Table 2B, Table 16 or SEQ ID NOs:1-1440; and
(b) comparing the expression profile from the sample to an expression profile of a control or standard.

45. The method of claim 44, wherein the plurality of targets comprises at least 5 targets selected from Table 2B, Table 16 or SEQ ID NOs:1-1440.

46. The method of claim 44, wherein the plurality of targets comprises at least 10 targets selected from Table 2B, Table 16 or SEQ ID NOs:1-1440.

47. The method of claim 44, wherein the plurality of targets comprises at least 15 targets selected from Table 2B, Table 16 or SEQ ID NOs:1-1440.

48. The method of claim 44, wherein the plurality of targets comprises SEQ ID NOs: 1-15.

49. The method of claim 44, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

50. The method of claim 44, further comprising providing diagnostic or prognostic information to the individual about the cancer based on the comparison.

51. The method of claim 44, further comprising diagnosing the individual with a cancer if the expression profile of the sample (a) deviates from the control or standard from a healthy individual or population of healthy individuals, or (b) matches the control or standard from an individual or population of individuals who have or have had the cancer.

52. The method of claim 44, further comprising predicting the susceptibility of the individual for developing a cancer based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

53. The method of claim 44, further comprising prescribing a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

54. The method of claim 44, further comprising altering a treatment regimen prescribed or administered to the individual based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

55. The method of claim 44, further comprising predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

56. The method of claim 55, wherein the deviation is the expression level of one or more targets from the sample is greater than the expression level of one or more targets from a control or standard derived from a healthy individual or population of healthy individuals.

57. The method of claim 55, wherein the deviation is the expression level of one or more targets from the sample is at least about 30% greater than the expression level of one or more targets from a control or standard derived from a healthy individual or population of healthy individuals.

58. The method of claim 55, wherein the deviation is the expression level of one or more targets from the sample is less than the expression level of one or more targets from a control or standard derived from a healthy individual or population of healthy individuals.

59. The method of claim 55, wherein the deviation is the expression level of one or more targets from the sample is at least about 30% less than the expression level of one or more targets from a control or standard derived from a healthy individual or population of healthy individuals.

60. The method of claim 44, further comprising using a machine to isolate the target or the probe from the sample.

61. The method of claim 44, further comprising contacting the sample with a label that specifically binds to the target, the probe, or a combination thereof.

62. The method of claim 44, further comprising amplifying the target, the probe, or any combination thereof.

63. The method of claim 44, further comprising sequencing the target, the probe, or any combination thereof.

64. A method of diagnosing cancer in an individual in need thereof, comprising:
(a) obtaining an expression profile from a sample obtained from the individual, wherein the expression profile comprises one or more targets selected from Table 2B, Table 16 or SEQ ID NOs:1-1440;
(b) comparing the expression profile from the sample to an expression profile of a control or standard; and
(c) diagnosing a cancer in the individual if the expression profile of the sample (i) deviates from the control or standard from a healthy individual or population of healthy individuals, or (ii) matches the control or standard from an individual or population of individuals who have or have had the cancer.

65. The method of claim 64, wherein the plurality of targets comprises at least 5 targets selected from Table 2B, Table 16 or SEQ ID NOs:1-1440.

66. The method of claim 64, wherein the plurality of targets comprises at least 10 targets selected from Table 2B, Table 16 or SEQ ID NOs:1-1440.

67. The method of claim 64, wherein the plurality of targets comprises at least 15 targets selected from Table 2B, Table 16 or SEQ ID NOs:1-1440.

68. The method of claim 64, wherein the plurality of targets comprises SEQ ID NOs: 1-15

69. The method of claim 64, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

70. The method of claim 64, further comprising a software module executed by a computer-processing device to compare the expression profiles.

71. The method of claim 64, further comprising using a machine to isolate the target or the probe from the sample.

72. The method of claim 64, further comprising contacting the sample with a label that specifically binds to the target, the probe, or a combination thereof.

73. The method of claim 64, further comprising amplifying the target, the probe, or any combination thereof.

74. The method of claim 64, further comprising sequencing the target, the probe, or any combination thereof.

75. A method of predicting whether an individual is susceptible to developing a cancer, comprising:
(a) obtaining an expression profile from a sample obtained from the individual, wherein the expression profile comprises one or more targets selected from Table 2B, Table 16 or SEQ ID NOs:1-1440;
(b) comparing the expression profile from the sample to an expression profile of a control or standard; and
(c) predicting the susceptibility of the individual for developing a cancer based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer.

76. The method of claim 75, wherein the plurality of targets comprises at least 5 targets selected from Table 2B, Table 16 or SEQ ID NOs:1-1440.

77. The method of claim 75, wherein the plurality of targets comprises at least 10 targets selected from Table 2B, Table 16 or SEQ ID NOs:1-1440.

78. The method of claim 75, wherein the plurality of targets comprises at least 15 targets selected from Table 2B, Table 16 or SEQ ID NOs:1-1440.

79. The method of claim 75, wherein the plurality of targets comprises SEQ ID NOs:1-15.

80. The method of claim 75, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

81. The method of claim 75, further comprising a software module executed by a computer-processing device to compare the expression profiles.

82. The method of claim 75, further comprising using a machine to isolate the target or the probe from the sample.

83. The method of claim 75, further comprising contacting the sample with a label that specifically binds to the target, the probe, or a combination thereof.

84. The method of claim 75, further comprising amplifying the target, the probe, or any combination thereof.

85. The method of claim 75, further comprising sequencing the target, the probe, or any combination thereof.

86. A method of predicting an individual's response to a treatment regimen for a cancer, comprising:
(a) obtaining an expression profile from a sample obtained from the individual, wherein the expression profile comprises one or more targets selected from Table 2B, Table 16 or SEQ ID NOs:1-1440;
(b) comparing the expression profile from the sample to an expression profile of a control or standard; and
(c) predicting the individual's response to a treatment regimen based on (a) the deviation of the expression profile of the sample from a control or standard derived from a healthy individual or population of healthy individuals, or (b) the similarity of the expression profiles of the sample and a control or standard derived from an individual or population of individuals who have or have had the cancer.

87. The method of claim 86, wherein the plurality of targets comprises at least 5 targets selected from Table 2B, Table 16 or SEQ ID NOs:1-1440.

88. The method of claim 86, wherein the plurality of targets comprises at least 10 targets selected from Table 2B, Table 16 or SEQ ID NOs:1-1440.

89. The method of claim 86, wherein the plurality of targets comprises at least 15 targets selected from Table 2B, Table 16 or SEQ ID NOs:1-1440.

90. The method of claim 86, wherein the plurality of targets comprises SEQ ID NOs:1-15.

91. The method of claim 86, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

92. The method of claim 86, further comprising a software module executed by a computer-processing device to compare the expression profiles.

93. The method of claim 86, further comprising using a machine to isolate the target or the probe from the sample.

94. The method of claim 86, further comprising contacting the sample with a label that specifically binds to the target, the probe, or a combination thereof.

95. The method of claim 86, further comprising amplifying the target, the probe, or any combination thereof.

96. The method of claim 86, further comprising sequencing the target, the probe, or any combination thereof.

97. A method of prescribing a treatment regimen for a cancer to an individual in need thereof, comprising:
(a) obtaining an expression profile from a sample obtained from the individual, wherein the expression profile comprises one or more targets selected from Table 2B, Table 16 or SEQ ID NOs:1-1440;
(b) comparing the expression profile from the sample to an expression profile of a control or standard; and
(c) prescribing a treatment regimen based on (i) the deviation of the expression profile of the sample from a control or standard derived from a healthy individual or population of healthy individuals, or (ii) the similarity of the expression profiles of the sample and a control or standard derived from an individual or population of individuals who have or have had the cancer.

98. The method of claim 97, wherein the plurality of targets comprises at least 5 targets selected from Table 2B, Table 16 or SEQ ID NOs:1-1440.

99. The method of claim 97, wherein the plurality of targets comprises at least 10 targets selected from Table 2B, Table 16 or SEQ ID NOs:1-1440.

100. The method of claim 97, wherein the plurality of targets comprises at least 15 targets selected from Table 2B, Table 16 or SEQ ID NOs:1-1440.

101. The method of claim 97, wherein the plurality of targets comprises SEQ ID NOs:1-15.

102. The method of claim 97, wherein the cancer is selected from the group consisting of bladder cancer, skin cancer, lung cancer, colon cancer, pancreatic cancer, prostate cancer, liver cancer, thyroid cancer, ovarian cancer, uterine cancer, breast cancer, cervical cancer, kidney cancer, epithelial carcinoma, squamous carcinoma, basal cell carcinoma, melanoma, papilloma, and adenomas.

103. The method of claim 97, further comprising a software module executed by a computer-processing device to compare the expression profiles.

104. The method of claim 97, further comprising using a machine to isolate the target or the probe from the sample.

105. The method of claim 97, further comprising contacting the sample with a label that specifically binds to the target, the probe, or a combination thereof.

106. The method of claim 97, further comprising amplifying the target, the probe, or any combination thereof.

107. The method of claim 97, further comprising sequencing the target, the probe, or any combination thereof.

108. The method of claim 97, further comprising converting the expression levels of the target sequences into a likelihood score that indicates the probability that a biological sample is from a patient who will exhibit no evidence of disease, who will exhibit systemic cancer, or who will exhibit cancer recurrence.

109. The method of claim 97, wherein the target sequences are differentially expressed the cancer.

110. The method of claim 109, wherein the differential expression is dependent on aggressiveness.

111. The method of claim 97, wherein the expression profile is determined by a method selected from the group consisting of RT-PCR, Northern blotting, ligase chain reaction, array hybridization, and a combination thereof.

112. A kit for analyzing a cancer, comprising:
(a) a probe set comprising a plurality of target sequences, wherein the plurality of target sequences comprises at least one target sequence listed in Table 2B, Table 16 or SEQ ID NOs:1-1440; and
(b) a computer model or algorithm for analyzing an expression level and/or expression profile of the target sequences in a sample.

113. The kit of claim 112, further comprising a computer model or algorithm for correlating the expression level or expression profile with disease state or outcome.

114. The kit of claim 112, further comprising a computer model or algorithm for designating a treatment modality for the individual.

115. The kit of claim 112, further comprising a computer model or algorithm for normalizing expression level or expression profile of the target sequences.

116. The kit of claim 112, further comprising a computer model or algorithm comprising a robust multichip average (RMA), frozen robust multichip average (fRMA), Single Channel Array Normalization (SCAN), ComBat (Combining Batches of gene expression), probe logarithmic intensity error estimation (PLIER), non-linear fit (NLFIT) quantile-based, nonlinear normalization, or a combination thereof.

117. The kit of claim 112, wherein the cancer is a bladder cancer.

118. The kit of claim 112, wherein the plurality of targets comprises SEQ ID NOs:1-15.
